(19) United States
(12) Patent Application Publication
(10) Pub. No.: US 2026/0327237 A1
(43) Pub. Date: Oct. 22, 2026

(54) GPR52 MODULATORS

(71) Applicant: Phrenix Therapeutics Pty Ltd, Clayton (AU)

(72) Inventors: Paul Stupple, Clayton (AU); Michelle Camerino, Clayton (AU); YIva Bozikis, Clayton (AU); Trang Pham, Clayton (AU); David Shackleford, Clayton (AU); Christopher Langmead, Clayton (AU); Gregory Stewart, Clayton (AU); Jonathan Baell, Clayton (AU); David C. Tully, Cambridge, MA (US); Patrick S. Lee, Cambridge, MA (US); Christopher R. Smith, Cambridge, MA (US)

(21) Appl. No.: 19/636,248

(22) Filed: Apr. 1, 2026

Related U.S. Application Data
(60) Provisional application No. 63/783,719, filed on Apr. 4, 2025.
(60) Provisional application No. 63/905,920, filed on Oct. 27, 2025.

(51) Int. Cl. C07D 487/04; A61K 31/4985; A61K 31/538; A61K 31/5383; A61K 31/551; C07B 59/00; C07D 471/04; C07D 498/04

(52) U.S. Cl. CPC C07D 487/04; A61K 31/4985; A61K 31/538; A61K 31/5383; A61K 31/551; C07B 59/002; C07D 471/04; C07D 498/04

(57) ABSTRACT

This disclosure provides chemical entities (e.g., a compound, or a pharmaceutically acceptable salt thereof) that modulate (e.g., agonize or partially agonize or antagonize) G-protein coupled receptor 52 (sometimes referred to herein as “GPR52”). The chemical entities are useful, e.g., for treating a subject (e.g., a human) having a disease, disorder, or condition in which modulation (e.g., agonism, partial agonism or antagonism) of GPR52 activity is beneficial for the treatment or prevention of the underlying pathology and/or symptoms and/or progression of the disease, disorder, or condition (e.g., a psychiatric disorder, e.g., schizophrenia). In some embodiments, the modulation results in enhancement of (e.g., increases) existing levels (e.g., normal or below normal levels) of GPR52 activity. In some embodiments, the chemical entities described herein further modulate (e.g., increase) β-arrestin recruitment to GPR52 relative to what is observed with the native ligand. This disclosure also features compositions as well as other methods of using and making said chemical entities.

### CROSS-REFERENCES TO RELATED APPLICATIONS

This application claims the benefit under 35 U.S.C. § 119 (e) of U.S. Provisional Application No. 63/783,719, filed on Apr. 4, 2025, and U.S. Provisional Application No. 63/905,920, filed on Oct. 27, 2025, each of which is incorporated herein by reference in its entirety for all purposes.

### TECHNICAL FIELD

This disclosure provides chemical entities (e.g., a compound, or a pharmaceutically acceptable salt thereof) that modulate (e.g., agonize or partially agonize or antagonize) G-protein coupled receptor 52 (sometimes referred to herein as “GPR52”). The chemical entities are useful, e.g., for treating a subject (e.g., a human) having a disease, disorder, or condition in which modulation (e.g., agonism, partial agonism or antagonism) of GPR52 activity is beneficial for the treatment or prevention of the underlying pathology and/or symptoms and/or progression of the disease, disorder, or condition (e.g., a psychiatric disorder, e.g., schizophrenia). In some embodiments, the modulation results in enhancement of (e.g., increases) existing levels (e.g., normal or below normal levels) of GPR52 activity. In some embodiments, the chemical entities described herein further modulate (e.g., increase) β-arrestin recruitment to GPR52 relative to what is observed with the native ligand. This disclosure also features compositions as well as other methods of using and making said chemical entities.

### BACKGROUND

GPR52 is a G protein-coupled receptor that is enriched in the central nervous system. GPR52 is highly expressed in dopamine D2-expressing spiny projection neurons in the striatum and dopamine D1 expressing pyramidal neurons in the cortex (see, e.g., Komatsu, H.; Maruyama, M.; Yao, S. et al. PLOS One. 2014, 28, e90134 (“Komatsu”); and Spark, D. L.; Mao, M.; Ma, S.; Sarwar, M.; Nowell, C. J.; Shackleford, D. M.; Sexton, P. M.; Nithianantharajah, J.; Stewart, G. D.; Langmead, C. J. ACS Chem Neurosci. 2020, 11, 2066-2076 “Spark”). The GPR52 coupling profile stimulates adenylate cyclase through G alpha s G protein activation, which would theoretically functionally inhibit dopamine D2 receptor activation in the striatum, and enhance dopamine D1 receptor activation in the prefrontal cortex (see, e.g., Komatsu). GPR52 also recruits beta-arrestin 1 and 2, the latter of which is typically needed for appropriate activation of cognition-relevant substrates in cortical neurons (see, e.g., Hatzipantelis, C. J.; Lu, Y.; Spark, D. L.; Langmead, C. J.; Stewart, G. D. ACS Chem. Neurosci. 2020, 11, 2077-2084 “Hatzipantelis”).

WO 2021/181122 discloses GPR52 modulator compounds having the following formula:

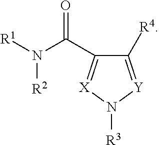

### SUMMARY

This disclosure provides chemical entities (e.g., a compound, or a pharmaceutically acceptable salt thereof) that modulate (e.g., agonize or partially agonize or antagonize) G-protein coupled receptor 52 (sometimes referred to herein as “GPR52”). The chemical entities are useful, e.g., for treating a subject (e.g., a human) having a disease, disorder, or condition in which modulation (e.g., agonism, partial agonism or antagonism) of GPR52 activity is beneficial for the treatment or prevention of the underlying pathology and/or symptoms and/or progression of the disease, disorder, or condition (e.g., a psychiatric disorder, e.g., schizophrenia). In some embodiments, the modulation results in enhancement of (e.g., increases) existing levels (e.g., normal or below normal levels) of GPR52 activity. In some embodiments, the chemical entities described herein further modulate (e.g., increase) β-arrestin recruitment to GPR52 relative to what is observed with the native ligand. This disclosure also features compositions as well as other methods of using and making said chemical entities.

In some embodiments, the compound of the disclosure is a compound of Formula (J):

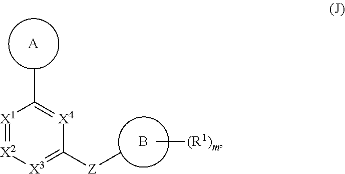

or a pharmaceutically acceptable salt thereof, wherein:
Ring A has the structure:

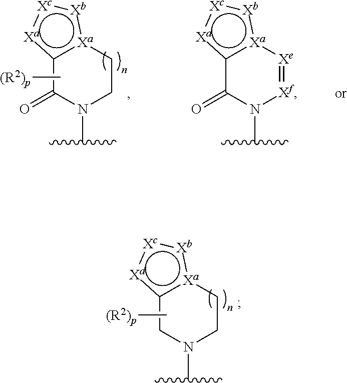

X^{a} is C or N;
X^{b}, X^{c} and X^{d} are each independently CH, C(R²), C(O), N, N(R^{N}), or O;
X^{e} and X^{f} are each independently CH, C(R²), or N;
wherein when X^{a} is C, at least two of X^{b}, X^{c} and X^{d} are independently N, N(R^{N}), or O, and when X^{a} is N, at least one of X^{b}, X^{c} and X^{d} is N and at least one of X^{b}, X^{c} and X^{d} is C(R²);
each R² is independently H, deuterium, halogen, amino, C1-C6 alkyl, C1-C6 deuteroalkyl, C1-C6 haloalkyl, C1-C6 alkoxy, C1-C6 deuteroalkoxy, C1-C6 hydroxyalkyl, C1-C6 alkylamino, C2-C6 hydroxyalkoxy, C2-C6 alkoxyalkoxy, C2-C6 hydroxyalkylamino, C1-C6 haloalkoxy, (benzyloxy)(C1-C6 alkyl), —C(═O)R^{2b}, —C(═O)OR^{2b}, —C(═O)—NR^{2b}R^{2c}, —NR^{2c}C(═O)R^{2b}, —NR^{2c}—C(═O)OR^{2b}, —NR^{2d}C(═O)—NR^{2b}R^{2c}, —SR^{2b}, —S(═O)R²⁶, —S(═O)₂R^{2b}, —S(═O)—NR^{2b}R^{2c}, —S(═O)₂—NR^{2b}R^{2c}, —S(═O)(═NH)—NR^{2b}R^{2c}, —NR^{2c}S(═O)R^{2b}, —NR^{2c}S(═O)₂R^{2b}, —NR^{2d}—S(═O)—NR^{2b}R^{2c}, —NR^{2d}S(═O)₂—NR^{2b}R^{2c}, —NR^{2d}s (═O)(═NH)—NR^{2b}R^{2c}, —(C1-C6 alkyl)-C(═O)R^{2b}, —(C1-C6 alkyl)-C(═O)OR^{2b}, —(C1-C6 alkyl)-C(═O)—NR^{2b}R^{2c}, —(C1-C6 alkyl)-S(═O)R^{2b}, —(C1-C6 alkyl)-S(═O)₂R^{2b}, —(C1-C6 alkyl)-S(═O)—NR^{2b}R^{2c}, —(C1-C6 alkyl)-S(═O)₂—NR^{2b}R^{2c}, —(C1-C6 alkyl)-S(═O)(═NH)—NR^{2b}R^{2c}, C3-6 cycloalkyl, heterocyclyl, —(C1-C6 alkyl) (C3-6 cycloalkyl), or —(C1-C6 alkyl) (heterocyclyl), wherein the cycloalkyl, heterocyclyl, (alkyl)cycloalkyl, and (alkyl) heterocyclyl are each independently substituted with 0, 1, 2, or 3 R^{2ª;}

each R^{2a} is independently deuterium, halogen, hydroxy, C1-C6 alkyl, C1-C6 deuteroalkyl, C1-C6 haloalkyl, C1-C6 alkoxy, C1-C6 deuteroalkoxy, C1-C6 hydroxyalkyl, C1-C6 alkylamino, C2-C6 hydroxyalkoxy, C2-C6 alkoxyalkoxy, C2-C6 hydroxyalkylamino, or C1-C6 haloalkoxy;
R^{2b}, R^{2c}, and R^{2d} are each independently H, C1-C6 alkyl, C1-C6 deuteroalkyl, or C1-C6 hydroxyalkyl;
or, alternatively, R^{2b} and R^{2c}, together with the nitrogen to which they are attached, form a heterocyclyl substituted with 0, 1, 2, or 3 halogen, hydroxy, C1-C6 alkyl, C1-C6 deuteroalkyl, or C1-C6 hydroxyalkyl;
each R^{N} is independently H, C1-C6 alkyl, C1-C6 deuteroalkyl, C1-C6 hydroxyalkyl, (benzyloxy)(C1-C6 alkyl), or a nitrogen protecting group;
subscript n is 1 or 2;
subscript p is 0, 1, 2, or 3;
Ring B is phenyl or heteroaryl;
each R¹ is independently halogen, oxo, cyano, C1-C6 alkyl, C1-C6 haloalkyl, C1-C6 alkoxy, C1-C6 alkylamino, C1-C6 haloalkoxy, C3-C6 cycloalkyl, heterocyclyl, or SF₅, wherein the cyclohexyl and heterocyclyl are each independently substituted with 0, 1, 2, or 3 R^{1a}; or
two adjacent R¹, together with the atoms to which they are attached, form a heterocyclyl or heteroaryl ring substituted with 0, 1, 2, or 3 R^{1a}, each R^{1a} is independently halogen, oxo, hydroxy, C1-C6 alkyl, C1-C6 haloalkyl, or C1-C6 hydroxyalkyl;
subscript m is 1, 2, or 3;
X¹ is N or CR^{X};
X² is N or CR^{X};
X³ is N or CR^{X};
X⁴ is N or CR^{X};
each R^{X} is independently H, halogen, oxo, C1-C6 alkyl, C1-C6 haloalkyl, C1-C6 alkoxy, or C1-C6 haloalkoxy;
Z is —O—, —S—, —S(═O)—, —S(O₂)—, —C(R^{A}R^{B})—, or —N(R^{C})—;
R^{A} and R^{B} are each independently hydrogen, halogen, hydroxyl, or C1-C6 alkyl; or R^{A} and R^{B}, together with the carbon atom to which they are attached, form ═CH₂, ═O, or a spirocyclopropyl;
or X⁴ and Z, together with the carbon atom to which they are attached, form

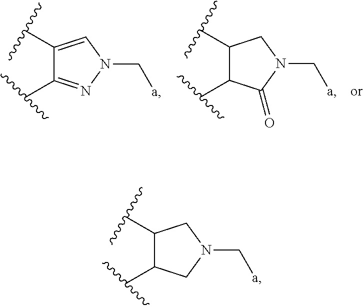

wherein “a” indicates the point of connection to

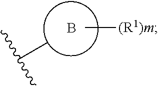

and
R^{C} is hydrogen or C1-C6 alkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

In some embodiments, the disclosure provides compounds of Formula (I):

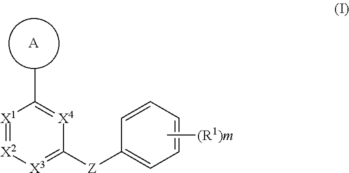

or a pharmaceutically acceptable salt thereof, wherein:
each R¹ is independently selected from halogen, cyano, C1-C6 alkyl, C1-C6 haloalkyl, C1-C6 alkoxy, and C1-C6 haloalkoxy; or two adjacent R¹, together with the carbon atoms to which they are attached, form a 5-membered heteroaryl ring optionally substituted with C1-C6 alkyl;
m is 1, 2, or 3;
X¹ is N or CR^{X};
X² is N or CR^{X};
X³ is N or CR^{X};
X⁴ is N or CR^{X};
each R^{X} is independently selected from H, halogen, C1-C6 alkyl, C1-C6 haloalkyl, C1-C6 alkoxy, and C1-C6 haloalkoxy;
Z is —O—, —S—, —S(═O)—, —S(O₂)—, —C(R^{A}R^{B})—, or —N(R^{C})—;
R^{A} and R^{B} are independently selected from hydrogen, halogen, hydroxyl, or C1-C6 alkyl;
or R^{A} and R^{B}, together with the carbon atom to which they are attached, form ═CH₂, ═O, or a spirocyclopropyl;
or X⁴ and Z, together with the carbon atom to which they are attached, form

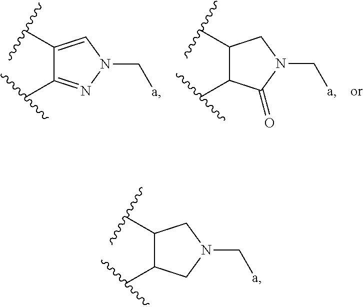

wherein “a” indicates the point of connection to

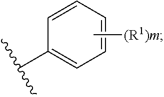

R^{C} is hydrogen or C1-C6 alkyl;
Ring A is selected from:

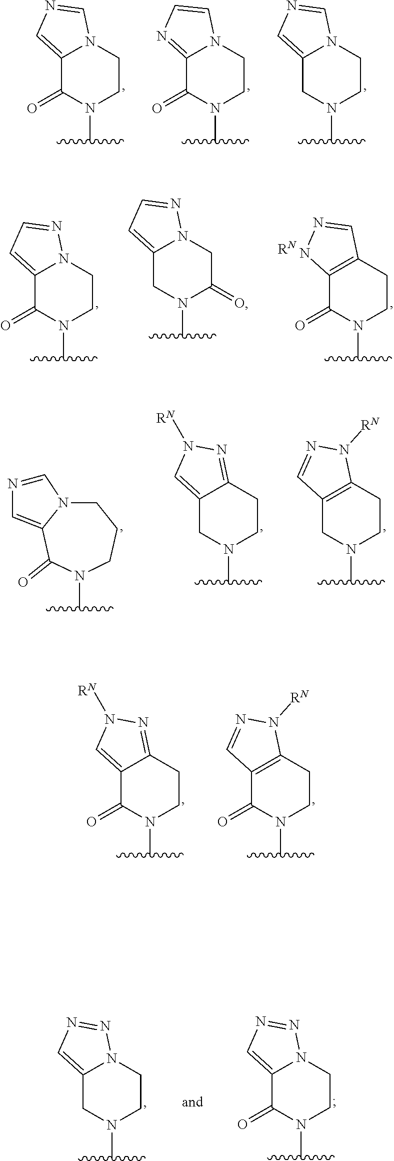

each optionally substituted with 1-3 substituents independently selected from deuterium, halogen, C1-C6 alkyl, C1-C6 deuteroalkyl, C1-C6 haloalkyl, C1-C6 alkoxy, C1-C6 deuteroalkoxy, C1-C6 hydroxyalkyl, and C1-C6 haloalkoxy; and
R^{N} is H, C1-C6 alkyl, C1-C6 deuteroalkyl, or a nitrogen protecting group.

Also provided herein are compositions, e.g., pharmaceutical compositions, which include a compound of Formula (J) and/or (I), or a pharmaceutically acceptable salt thereof, and a pharmaceutically acceptable carrier and/or excipient.

### DETAILED DESCRIPTION OF THE INVENTION

### I. General

This disclosure provides chemical entities (e.g., a compound, or a pharmaceutically acceptable salt thereof) that modulate (e.g., agonize or partially agonize or antagonize) G-protein coupled receptor 52 (sometimes referred to herein as “GPR52”). The chemical entities are useful, e.g., for treating a subject (e.g., a human) having a disease, disorder, or condition in which modulation (e.g., agonism, partial agonism or antagonism) of GPR52 activity is beneficial for the treatment or prevention of the underlying pathology and/or symptoms and/or progression of the disease, disorder, or condition (e.g., a psychiatric disorder, e.g., schizophrenia). In some embodiments, the modulation results in enhancement of (e.g., increases) existing levels (e.g., normal or below normal levels) of GPR52 activity. In some embodiments, the chemical entities described herein further modulate (e.g., increase) β-arrestin recruitment to GPR52 relative to what is observed with the native ligand. This disclosure also features compositions as well as other methods of using and making said chemical entities.

### II. Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “halo” refers to fluoro (F), chloro (Cl), bromo (Br), or iodo (I).

The term “hydroxy” refers to the substituent —OH.

The term “amino” refers to the substituent —NH₂.

The term “oxo” refers to the substituent ═O.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “alkylamino” refers to an amino, in which one or more hydrogen atoms is/are replaced with an alkyl.

The term “haloalkyl” refers to an alkyl, in which one or more hydrogen atoms is/are replaced with an independently selected halo.

The term “hydroxyalkyl” refers to an alkyl, in which one or more hydrogen atoms is/are replaced with hydroxyl.

The term “hydroxyalkylamino” refers to a hydroxyalkyl, in which one or more hydrogen atoms is/are replaced with an amino.

The term “deuteroalkyl” refers to an alkyl, in which one or more hydrogen atoms is/are replaced with deuterium.

The term “alkoxy” refers to an —O-alkyl radical (e.g., —OCH₃).

The term “haloalkoxy” refers to an —O-alkyl radical, in which one or more hydrogen atoms is/are replaced with an independently selected halo.

The term “deuteroalkoxy” refers to an —O-alkyl radical, in which in which one or

more hydrogen atoms is/are replaced with deuterium.

The term “5-member heteroaryl”, as used herein, means a an aromatic ring system having 5 ring atoms; wherein at least one ring in the system is a heteroatom independently selected from the group consisting of N, O, and S, which is unsubstituted or substituted with one or more substituents. Examples include thienyl, pyridinyl, furyl, oxazolyl, oxadiazolyl, pyrrolyl, imidazolyl, triazolyl, thiodiazolyl, pyrazolyl, isoxazolyl, and thiadiazolyl.

The term “spirocyclopropyl” refers to:

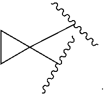 [SMILES: *C1(*)CC1]

As used herein, the term “nitrogen-protecting group” (PG) in a compound described herein to a group that protects a functional groups against unwanted secondary reactions, such as acylations, etherifications, esterifications, oxidations, solvolysis and similar reactions. A variety of deprotection conditions are available for removing the protecting groups described here. Depending on the protecting group employed, the skilled person would know how to remove the protecting group to obtain the free amine NH₂ group by reference to known procedures. These include reference to organic chemistry textbooks and literature procedures such as J. F. W. McOmie, “Protective Groups in Organic Chemistry”, T. W. Greene and P. G. M. Wuts, “Greene's Protective Groups in Organic Synthesis”, and in “Methoden der organischen Chemie” (Methods of Organic Chemistry).

In addition, atoms making up the compounds of the present embodiments are intended to include all isotopic forms of such atoms. The compounds provided herein encompass various stereochemical forms. The compounds described herein include all stereoisomers, including diastereoisomers, atropisomers, enantiomers, mixtures thereof and racemic mixtures, which arise because of structural asymmetry in certain compounds. Pharmaceutically acceptable salts thereof of these stereoisomeric forms are also included.

The term “acceptable” with respect to a formulation, composition or ingredient, as used herein, means having no persistent detrimental effect on the general health of the subject being treated.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CR^{C} Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

Generally, a receptor exists in an active (Ra) and an inactive (Ri) conformation. Certain compounds that affect the receptor can alter the ratio of Ra to Ri (Ra/Ri). For example, a full agonist increases the ratio of Ra/Ri and can cause a “maximal”, saturating effect. A partial agonist, when bound to the receptor, gives a response that is lower than that elicited by a full agonist (e.g., an endogenous agonist). Thus, the Ra/Ri for a partial agonist is less than for a full agonist. However, the potency of a partial agonist may be greater or less than that of the full agonist.

The term “GPR52 modulator,” including the compounds encompassed by Formula (J) and/or (I), as used herein refers to any compound which binds to and modulates the function of the GPR52 receptor. The term “modulator” includes, but is not limited to, agonists, partial agonists, and antagonists.

An “agonist” of GPR52 includes compounds that, at the protein level, directly bind or modify GPR52 such that an activity of GPR52 is increased, e.g., by activation, stabilization, altered distribution, or otherwise relative to GPR52 activity in absence of ligand.

Certain compounds described herein can agonize GPR52 to a lesser extent than a GPR52 full agonist. These compounds are sometimes referred to herein as “partial agonists” of GPR52.

An “antagonist” of GPR52 includes compounds that, at the protein level, directly bind or modify GPR52 such that an activity of GPR52 is decreased, e.g., by inhibition, blocking or dampening agonist-mediated responses, altered distribution, or otherwise relative to GPR52 activity in absence of ligand.

The details of one or more embodiments of the invention are set forth in the accompanying drawings and in the description below. Other features and advantages will be apparent from the description and from the claims.

### III. Compounds

In some embodiments, the compound of the disclosure is a compound of Formula (J):

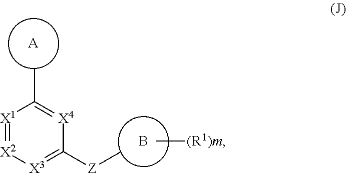

or a pharmaceutically acceptable salt thereof, wherein:
Ring A has the structure:

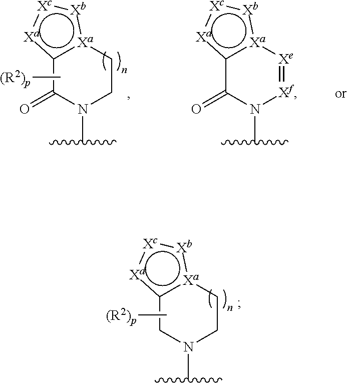

X^{a} is C or N;
X^{b}, X^{c} and X^{d} are each independently CH, C(R²), C(O), N, N(R^{N}), or O;
X^{e} and X^{f} are each independently CH, C(R²), or N;
wherein when X^{a} is C, at least two of X^{b}, X^{c} and X^{d} are independently N, N(R^{N}), or O, and when X^{a} is N, at least one of X^{b}, X^{c} and X^{d} is N and at least one of X^{b}, X^{c} and X^{d} is C(R²);
each R² is independently H, deuterium, halogen, amino, C1-C6 alkyl, C1-C6 deuteroalkyl, C1-C6 haloalkyl, C1-C6 alkoxy, C1-C6 deuteroalkoxy, C1-C6 hydroxyalkyl, C1-C6 alkylamino, C2-C6 hydroxyalkoxy, C2-C6 alkoxyalkoxy, C2-C6 hydroxyalkylamino, C1-C6 haloalkoxy, (benzyloxy)(C1-C6 alkyl), —C(═O)R^{2b}, —C(═O)OR^{2b}, —C(═O)—NR^{2b}R^{2c}, —NR^{2c}C(═O)R²⁶, —NR^{2c}—C(═O)OR²⁶, —NR^{2d}C(═O)—NR^{2b}R^{2c}, —SR^{2b}, —S(═O)R^{2b}, —S(═O)₂R^{2b}, —S(═O)—NR^{2b}R^{2c}, —S(═O)₂—NR^{2b}R^{2c}, —S(═O)(═NH)—NR^{2b}R^{2c}, —NR^{2e}S(═O)R^{2b}, —NR^{2c}S(═O)₂R^{2b}, —NR^{2d}—S(═O)—NR^{2b}R^{2c}, —NR^{2d}S(═O)₂—NR^{2b}R^{2c}, —NR^{2d}S(═O)(═NH)—NR^{2b}R^{2c}, —(C1-C6 alkyl)-C(═O)R^{2b}, —(C1-C6 alkyl)-C(═O)OR^{2b}, —(C1-C6 alkyl)-C(═O)—NR^{2b}R^{2c}, —(C1-C6 alkyl)-S(═O)R^{2b}, —(C1-C6 alkyl)-S(═O)₂R^{2b}, —(C1-C6 alkyl)-S(═O)—NR^{2b}R^{2c}, —(C1-C6 alkyl)-S(═O)₂—NR^{2b}R^{2c}, —(C1-C6 alkyl)-S(═O)(═NH)—NR^{2b}R^{2c}, C3-6 cycloalkyl, heterocyclyl, —(C1-C6 alkyl) (C3-6 cycloalkyl), or —(C1-C6 alkyl) (heterocyclyl), wherein the cycloalkyl, heterocyclyl, (alkyl)cycloalkyl, and (alkyl) heterocyclyl are each independently substituted with 0, 1, 2, or 3 R^{2a};
each R^{2a} is independently deuterium, halogen, hydroxy, C1-C6 alkyl, C1-C6 deuteroalkyl, C1-C6 haloalkyl, C1-C6 alkoxy, C1-C6 deuteroalkoxy, C1-C6 hydroxyalkyl, C1-C6 alkylamino, C2-C6 hydroxyalkoxy, C2-C6 alkoxyalkoxy, C2-C6 hydroxyalkylamino, or C1-C6 haloalkoxy;
R^{2b}, R^{2c}, and R^{2d} are each independently H, C1-C6 alkyl, C1-C6 deuteroalkyl, or C1-C6 hydroxyalkyl;
or, alternatively, R^{2b} and R^{2c}, together with the nitrogen to which they are attached, form a heterocyclyl substituted with 0, 1, 2, or 3 halogen, hydroxy, C1-C6 alkyl, C1-C6 deuteroalkyl, or C1-C6 hydroxyalkyl;
each R^{N} is independently H, C1-C6 alkyl, C1-C6 deuteroalkyl, C1-C6 hydroxyalkyl, (benzyloxy)(C1-C6 alkyl), or a nitrogen protecting group;
subscript n is 1 or 2;
subscript p is 0, 1, 2, or 3;
Ring B is phenyl or heteroaryl;
each R¹ is independently halogen, oxo, cyano, C1-C6 alkyl, C1-C6 haloalkyl, C1-C6 alkoxy, C1-C6 alkylamino, C1-C6 haloalkoxy, C3-C6 cycloalkyl, heterocyclyl, or SF₅, wherein the cyclohexyl and heterocyclyl are each independently substituted with 0, 1, 2, or 3 R^{1a}, or
two adjacent R¹, together with the atoms to which they are attached, form a heterocyclyl or heteroaryl ring substituted with 0, 1, 2, or 3 R^{1a},
each R^{1a} is independently halogen, oxo, hydroxy, C1-C6 alkyl, C1-C6 haloalkyl, or C1-C6 hydroxyalkyl;
subscript m is 1, 2, or 3;
X¹ is N or CR^{X};
X² is N or CR^{X};
X³ is N or CR^{X};
X⁴ is N or CR^{X};
each R^{X} is independently H, halogen, oxo, C1-C6 alkyl, C1-C6 haloalkyl, C1-C6 alkoxy, or C1-C6 haloalkoxy;
Z is —O—, —S—, —S(═O)—, —S(O₂)—, —C(R^{A}R^{B})—, or —N(R^{C})—;
R^{A} and R^{B} are each independently hydrogen, halogen, hydroxyl, or C1-C6 alkyl;
or R^{A} and R^{B}, together with the carbon atom to which they are attached, form —CH₂, ═O, or a spirocyclopropyl;
or X⁴ and Z, together with the carbon atom to which they are attached, form

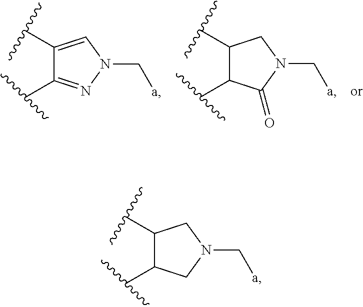

wherein “a” indicates the point of connection to

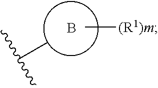

and
R^{C} is hydrogen or C1-C6 alkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

In some embodiments, the compound is a compound of Formula (J):

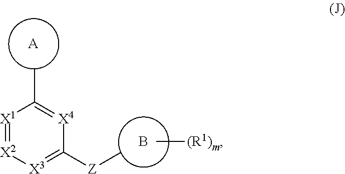

or a pharmaceutically acceptable salt thereof, wherein:
Ring A has the structure:

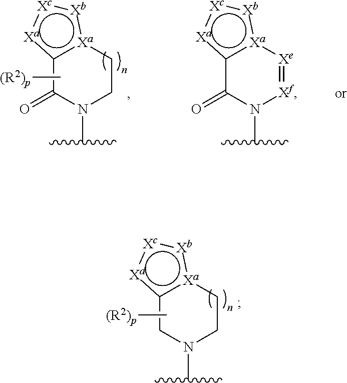

X^{a} is C or N;
X^{b}, X^{c} and X^{d} are each independently CH, C(R²), C(O), N, N(R^{N}), or O;
X^{e} and X^{f} are each independently CH, C(R²), or N;
wherein when X^{a} is C, at least two of X^{b}, X^{c} and X^{d} are independently N, N(R^{N}), or O, and when X^{a} is N, at least one of X^{b}, X^{c} and X^{d} is N and at least one of X^{b}, X^{c} and X^{d} is C(R²);
each R² is independently deuterium, halogen, amino, C1-C6 alkyl, C1-C6 deuteroalkyl, C1-C6 haloalkyl, C1-C6 alkoxy, C1-C6 deuteroalkoxy, C1-C6 hydroxyalkyl, C1-C6 alkylamino, C2-C6 hydroxyalkoxy, C2-C6 alkoxyalkoxy, C2-C6 hydroxyalkylamino, C1-C6 haloalkoxy, (benzyloxy)(C1-C6 alkyl), —C(═O)R^{2b}, —C(═O)OR^{2b}, —C(═O)—NR^{2b}R^{2b}, —S(═O)₂—NR^{2b}R^{2c}, C3-6 cycloalkyl, or heterocyclyl, wherein the cycloalkyl and heterocyclyl are each independently substituted with 0, 1, 2, or 3 R^{2ª;}
each R^{2a} is independently deuterium, halogen, hydroxy, C1-C6 alkyl, C1-C6 deuteroalkyl, C1-C6 haloalkyl, C1-C6 alkoxy, C1-C6 deuteroalkoxy, C1-C6 hydroxyalkyl, C1-C6 alkylamino, C2-C6 hydroxyalkoxy, C2-C6 alkoxyalkoxy, C2-C6 hydroxyalkylamino, or C1-C6 haloalkoxy;
R^{2b} and R^{2c} are independently H, C1-C6 alkyl, C1-C6 deuteroalkyl, or C1-C6 hydroxyalkyl;
each R^{N} is independently H, C1-C6 alkyl, C1-C6 deuteroalkyl, C1-C6 hydroxyalkyl, (benzyloxy)(C1-C6 alkyl), or a nitrogen protecting group;
subscript n is 1 or 2;
subscript p is 0, 1, 2, or 3;
Ring B is phenyl or heteroaryl;
each R¹ is independently halogen, oxo, cyano, C1-C6 alkyl, C1-C6 haloalkyl, C1-C6 alkoxy, C1-C6 alkylamino, C1-C6 haloalkoxy, C3-C6 cycloalkyl, heterocyclyl, or SF₅, wherein the cyclohexyl and heterocyclyl are each independently substituted with 0, 1, 2, or 3 R^{1a}, or
two adjacent R¹, together with the atoms to which they are attached, form a heterocyclyl or heteroaryl ring substituted with 0, 1, 2, or 3 R^{1a};
each R^{1a} is independently halogen, oxo, hydroxy, C1-C6 alkyl, or C1-C6 haloalkyl;
subscript m is 1, 2, or 3;
X¹ is N or CR^{X};
X² is N or CR^{X};
X³ is N or CR^{X};
X⁴ is N or CR^{X};
each R^{X} is independently H, halogen, oxo, C1-C6 alkyl, C1-C6 haloalkyl, C1-C6 alkoxy, or C1-C6 haloalkoxy;
Z is —O—, —S—, —S(═O)—, —S(O₂)—, —C(R^{A}R^{B})—, or —N(R^{C})—; R^{A} and R^{B} are each independently hydrogen, halogen, hydroxyl, or C1-C6 alkyl;
or R^{A} and R^{B}, together with the carbon atom to which they are attached, form ═CH₂, ═O, or a spirocyclopropyl;
or X⁴ and Z, together with the carbon atom to which they are attached, form

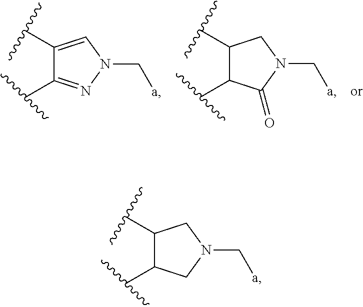

wherein “a” indicates the point of connection to

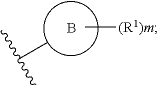

and
R^{C} is hydrogen or C1-C6 alkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

In some embodiments, the compound of the disclosure, e.g., a compound of Formula (J), is a compound of Formula (I):

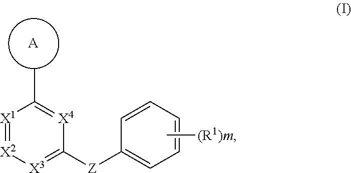

or a pharmaceutically acceptable salt thereof, wherein:
each R¹ is independently selected from halogen, cyano, C1-C6 alkyl, C1-C6 haloalkyl, C1-C6 alkoxy, and C1-C6 haloalkoxy; or
two adjacent R¹, together with the carbon atoms to which they are attached, form a 5-membered heteroaryl ring optionally substituted with C1-C6 alkyl;
m is 1, 2, or 3;
X¹ is N or CR^{X};
X² is N or CR^{X};
X³ is N or CR^{X};
X⁴ is N or CR^{X};
each R^{X} is independently selected from H, halogen, C1-C6 alkyl, C1-C6 haloalkyl, C1-C6 alkoxy, and C1-C6 haloalkoxy;
Z is —O—, —S—, —S(═O)—, —S(O₂)—, —C(R^{A}R^{B})—, or —N(R^{C})—;
R^{A} and R^{B} are independently selected from hydrogen, halogen, hydroxyl, or C1-C6 alkyl;
or R^{A} and R^{B}, together with the carbon atom to which they are attached, form ═CH₂, ═O, or a spirocyclopropyl;
or X⁴ and Z, together with the carbon atom to which they are attached, form

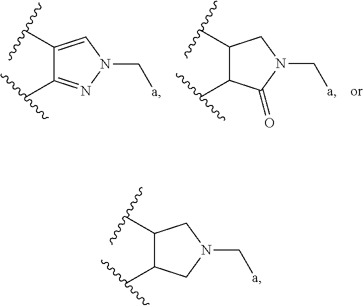

wherein “a” indicates the point of connection to

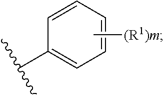

R^{C} is hydrogen or C1-C6 alkyl;
Ring A is selected from:

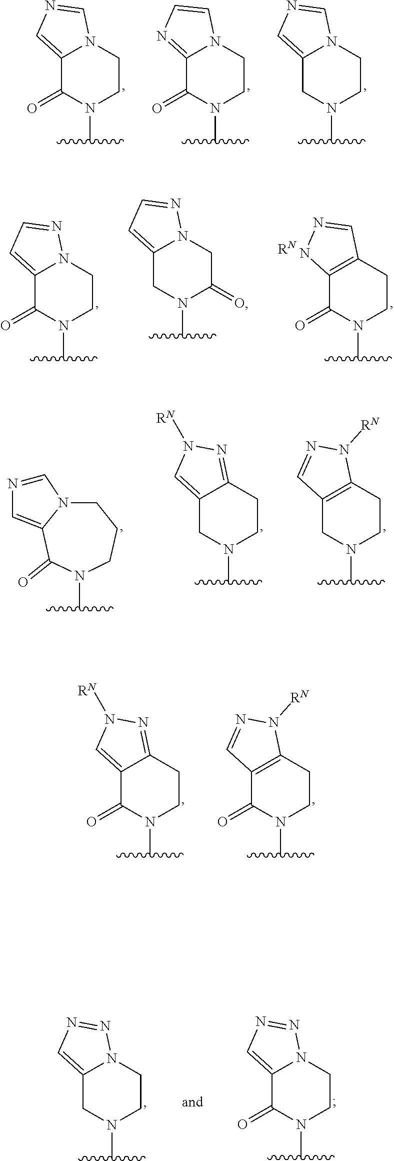

each optionally substituted with 1-3 substituents independently selected from deuterium, halogen, C1-C6 alkyl, C1-C6 deuteroalkyl, C1-C6 haloalkyl, C1-C6 alkoxy, C1-C6 deuteroalkoxy, C1-C6 hydroxyalkyl, and C1-C6 haloalkoxy; and
R^{N} is H, C1-C6 alkyl, C1-C6 deuteroalkyl, or a nitrogen protecting group.

In some embodiments, nitrogen-protecting groups include aryl, preferably phenyl, or a heterocyclic group (e.g., benzyl, cumyl, benzhydryl, pyrrolidinyl, trityl, pyrrolidinylmethyl, 1-methyl-1,1-dimethylbenzyl, (phenyl)methylbenzene) wherein the aryl ring or the heterocyclic group is unsubstituted or substituted by one or more, e.g. two or three, residues, e.g. selected from the group consisting of C₁-C₇alkyl, hydroxy, C₁-C₇alkoxy (e.g. para-methoxy benzyl (PMB)), C₂-C₈-alkanoyl-oxy, halogen, nitro, cyano, and CF₃.

In some embodiments, nitrogen-protecting groups include aryl-C₁-C₂-alkoxycarbonyl (preferably phenyl-C₁-C₂-alkoxycarbonyl (e.g. benzyloxycarbonyl (Cbz), benzyloxymethyl (BOM), pivaloyloxymethyl (POM)), C₁-C₁₀-alkenyloxycarbonyl, C1-C6alkylcarbonyl (e.g. acetyl or pivaloyl), C₆-C₁₀-arylcarbonyl; C₁-C₆-alkoxycarbonyl (e.g. tert-butoxycarbonyl (Boc), methylcarbonyl, trichloroethoxycarbonyl (Troc), pivaloyl (Piv), allyloxycarbonyl), C₆-C₁₀-arylC1-C6-alkoxycarbonyl (e.g. 9-fluorenylmethyloxycarbonyl (Fmoc)), allyl or cinnamyl, sulfonyl or sulfenyl, succinimidyl group, silyl groups (e.g. triarylsilyl, trialkylsilyl, triethylsilyl (TES), trimethylsilylethoxymethyl (SEM), trimethylsilyl (TMS), triisopropylsilyl or tert-butyldimethylsilyl).

In some embodiments, the nitrogen-protecting group is C₁-C₆-alkoxycarbonyl (e.g. tert-butoxycarbonyl (Boc), methyloxycarbonyl, trichloroethoxycarbonyl (Troc), pivaloyl (Piv), allyloxycarbonyl), e.g., the nitrogen-protecting group can be tert-butoxycarbonyl.

In some embodiments, the nitrogen-protecting group is a C₁-C₆-alkoxycarbonyl (e.g. tert-butoxycarbonyl or t-butyl carbamate (Boc), methyloxycarbonyl or methyl carbamate, ethyl carbamate, 9-fluorophenylmethyl carbamate (Fmoc) and analogs thereof, 2,2,2-trichloroethyl carbamate trichloroethoxycarbonyl (Troc), 2-trimethylsilylethyl carbamate (Teoc), pivaloyl (Piv), allyloxycarbonyl or allyl carbamate (Alloc), benzyl carbamate (Cbz)) or an amide protecting group e.g. COCF₃ (trifluoroacetamide), or N-allyl or N-benzyl and analogs thereof.; e.g., the nitrogen-protecting group is tert-butoxycarbonyl.

In certain embodiments, the nitrogen protecting group is selected from Carbobenzyloxy (Cbz); p-Methoxybenzyl carbonyl (Moz or MeOZ); tert-Butyloxycarbonyl (BOC) group; 9-Fluorenylmethyloxycarbonyl (FMOC); Acetyl (Ac); Trifluoroacetyl; Benzoyl (Bz); Benzyl (Bn); Carbamates; p-Methoxybenzyl (PMB); 3,4-Dimethoxybenzyl (DMPM); p-methoxyphenyl (PMP) group; and Tosyl (Ts) group.

In some embodiments, the compound of the present disclosure, e.g., a compound of Formula (J) and/or (I), has the structure of Formula (II):

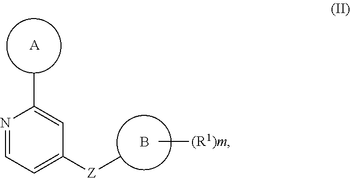

or a pharmaceutically acceptable salt thereof.

In some embodiments, Ring A is

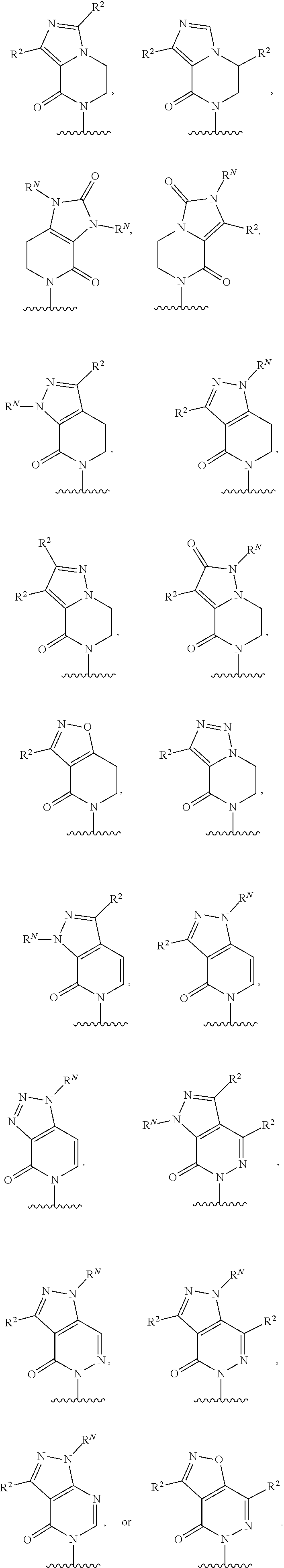

In some embodiments, Ring A is

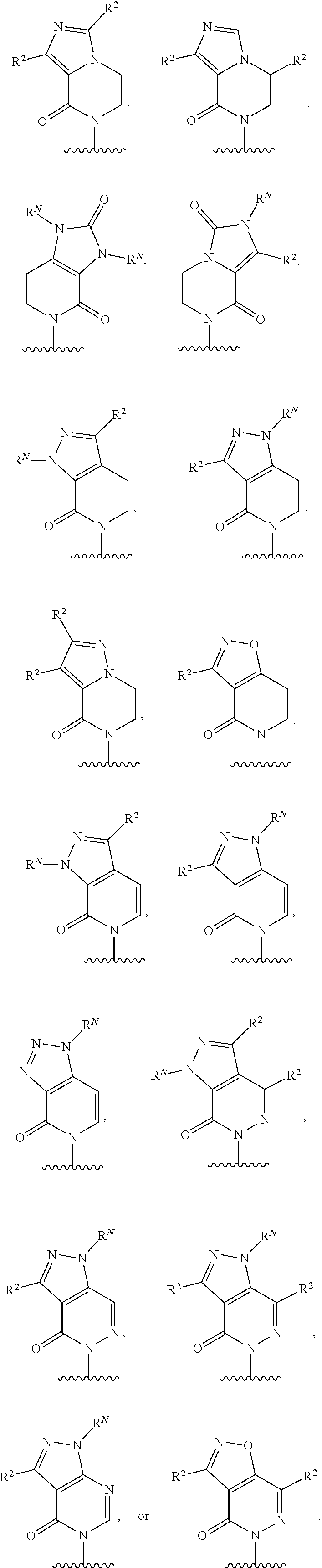

In some embodiments, Ring A is

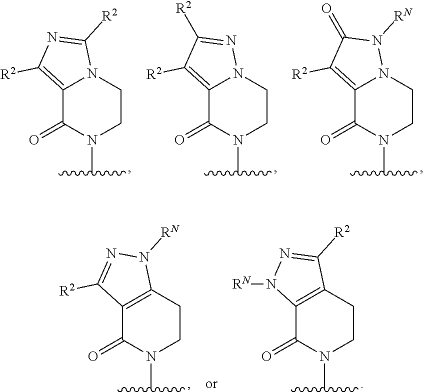

In some embodiments, Ring A is

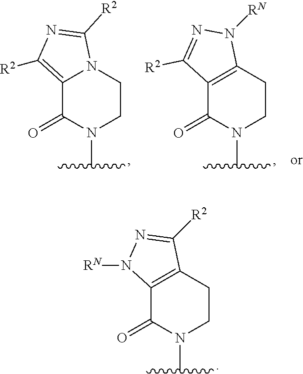

In some embodiments, Ring A is

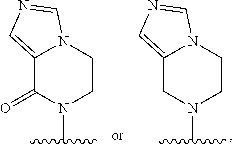 [SMILES: *N1CCn2cncc2C1=O ; *N1CCn2cncc2C1]

each of which is substituted with 0-3 substituents independently selected from deuterium, halogen, C1-C6 alkyl, C1-C6 deuteroalkyl, C1-C6 haloalkyl, C1-C6 alkoxy, C1-C6 deuteroalkoxy, and C1-C6 haloalkoxy.

In certain embodiments, Ring A is

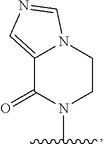 [SMILES: *N1CCn2cncc2C1=O]

which is substituted with 0-3 substituents independently selected from deuterium, halogen, C1-C6 alkyl, C1-C6 deuteroalkyl, C1-C6 haloalkyl, C1-C6 alkoxy, C1-C6 deuteroalkoxy, and C1-C6 haloalkoxy. For example, Ring A can be

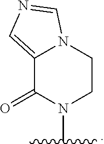 [SMILES: *N1CCn2cncc2C1=O]

In some embodiments, Ring A is:

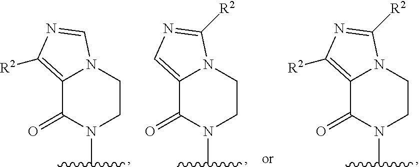

wherein each occurrence of R² is independently selected from deuterium, halogen, C1-C6 alkyl, C1-C6 deuteroalkyl, C1-C6 haloalkyl, C1-C6 alkoxy, C1-C6 deuteroalkoxy, and C1-C6 haloalkoxy.

In certain embodiments, Ring A is:

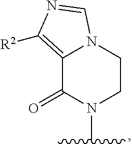

wherein R² is selected from deuterium, halogen, C1-C6 alkyl, C1-C6 deuteroalkyl, C1-C6 haloalkyl, C1-C6 alkoxy, C1-C6 deuteroalkoxy, and C1-C6 haloalkoxy.

In some embodiments, R² is C1-C6 alkyl. For example, R² can be CH₃.

In some embodiments, R² is C1-C6 haloalkyl. For example, R² can be CF₃.

In some embodiments, R² is C1-C6 alkoxy. For example, R² can be OCH₃ or OCH₂CH₃.

In certain embodiments, Ring A is

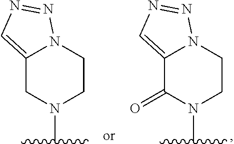 [SMILES: *N1CCn2nncc2C1 ; *N1CCn2nncc2C1=O]

each of which is substituted with 0-3 R² substituents independently selected from deuterium, halogen, C1-C6 alkyl, C1-C6 deuteroalkyl, C1-C6 haloalkyl, C1-C6 alkoxy, C1-C6 deuteroalkoxy, and C1-C6 haloalkoxy.

In certain embodiments, Ring A is

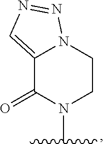 [SMILES: *N1CCn2nncc2C1=O]

which is substituted with 0-3 R² substituents independently selected from deuterium, halogen, C1-C6 alkyl, C1-C6 deuteroalkyl, C1-C6 haloalkyl, C1-C6 alkoxy, C1-C6 deuteroalkoxy, and C1-C6 haloalkoxy.

In certain embodiments, Ring A is:

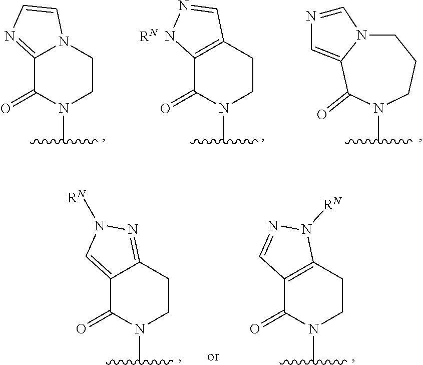

each of which is substituted with 0-3 R² substituents independently selected from deuterium, halogen, C1-C6 alkyl, C1-C6 deuteroalkyl, C1-C6 haloalkyl, C1-C6 alkoxy, C1-C6 deuteroalkoxy, and C1-C6 haloalkoxy.

In some embodiments, each R² is independently C1-C3 alkyl, C1-C3 hydroxyalkyl, C1-C3 alkylamino, C1-C3 hydroxyalkoxy, C1-C3 hydroxyalkylamino, —S(═O)—NR^{2b}R^{2c}, —C(═O)—NR^{2b}R^{2c}, —(C1-C3 alkyl)-C(═O)OR^{2b}, C3-C4 cycloalkyl, or a 4-5 membered heterocyclyl having 1 heteroatom N, wherein the cycloalkyl and heterocyclyl are each independently substituted with 1 R^{2a} substituent; R^{2a} is hydroxy or C1-C3 hydroxyalkyl; R^{2b} and R^{2c} are each H, C1-C3 alkyl, or C1-C3 hydroxyalkyl; or, alternatively, R^{2b} and R^{2c}, together with the nitrogen to which they are attached, form a heterocyclyl substituted with hydroxy or C1-C3 hydroxyalkyl; and R^{N} is H, C1-C3 alkyl, or C1-C3 hydroxyalkyl.

In some embodiments, each R² is C1-C3 alkyl, C1-C3 hydroxyalkyl, C1-C3 hydroxyalkoxy, C1-C3 hydroxyalkylamino, —C(═O)—NR^{2b}R^{2c}, C3-C4 cycloalkyl, or a 4-5 membered heterocyclyl having 1 heteroatom N, wherein the cycloalkyl and heterocyclyl are each independently substituted with 1 R^{2a} substituent; R^{2a} is hydroxy or C1-C3 hydroxyalkyl; R^{2b} and R^{2c} are each H; and R^{N} is C1-C3 alkyl, or C1-C3 hydroxyalkyl.

In some embodiments, 1-2 of X¹, X², X³, and X⁴ are N.

In certain embodiments, X¹ is N.

In some embodiments, X¹ is CR^{X}. In certain of these embodiments, R^{X} is H.

In certain embodiments, X³ is N.

In certain embodiments, X⁴ is CR^{X}. In certain of these embodiments, R^{X} is H. In other embodiments, R^{X} is OH.

In certain embodiments, X² is CR^{X}. In certain of these embodiments, R^{X} is H. In other embodiments, R^{X} is C1-C3 haloalkyl, e.g., R^{X} can be CF₃. In still other embodiments, R^{X} is C1-C3 alkoxy, e.g., R^{X} can be OCH₃.

In some embodiments, X² is N.

In certain embodiments, X¹ is N, and X³ and X⁴ are CR^{X} (embodiment A).

In certain embodiments of (A), one or both occurrences of R^{X} are H (e.g., both occurrences) (A1).

In certain embodiments of (A) and (A1), X² is CR^{X}. In certain of these embodiments, R^{X} is H. In other embodiments, R^{X} is C1-C3 haloalkyl, e.g., R^{X} can be CF₃. In still other embodiments, R^{X} is C1-C3 alkoxy, e.g., R^{X} can be OCH₃.

In certain embodiments, X³ is N, and X¹ and X⁴ are CR^{X} (embodiment B).

In certain embodiments of (B), one or both occurrences of R^{X} are H (e.g., both occurrences) (B1).

In certain embodiments of (B) and (B1), X² is CR^{X}. In some embodiments, R^{X} is H. In some embodiments, R^{X} is C1-C3 haloalkyl, e.g., R^{X} can be CF₃. In still other embodiments, R^{X} is C1-C3 alkoxy, e.g., R^{X} can be OCH₃.

In certain embodiments, X¹ and X³ are N, and X⁴ is CR^{X} (embodiment C).

In certain embodiments of (C), R^{X} is H (C1).

In certain embodiments of (C) and (C1), X² is CR^{X}. In certain of these embodiments, R^{X} is H. In some embodiments, R^{X} is C1-C3 haloalkyl, e.g., R^{X} can be CF₃. In some embodiments, R^{X} is C1-C3 alkoxy, e.g., R^{X} can be OCH₃.

In certain embodiments, X¹ and X² are N, and each of X³ and X⁴ is an independently selected CR^{X} (embodiment D).

In certain embodiments of (D), one or both occurrences of R^{X} are H (e.g., both occurrences) (D1).

In certain embodiments of (D) or (D1), one occurrence of R^{X} is H, and the other occurrence of R^{X} is C1-C3 haloalkyl, e.g., R^{X} can be CF₃, or the other occurrence of R^{X} is C1-C3 alkoxy, e.g., R^{X} can be OCH₃ (D2).

In certain embodiments of (D), each occurrence of R^{X} is selected from C1-C3 haloalkyl, e.g., R^{X} can be CF₃; and C1-C3 alkoxy, e.g., R^{X} can be OCH₃ (D3).

In certain embodiments, X⁴ and Z, together with the carbon atom to which they are attached, form

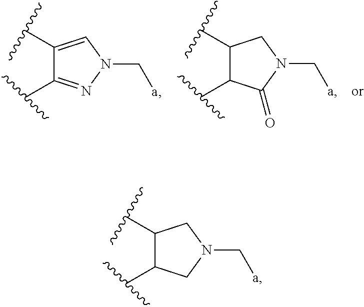

wherein “a” indicates the point of connection to

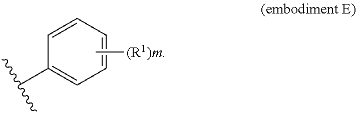

In certain embodiments of (E), X¹ is N (E1).

In certain embodiments of (E) and (E1), each of X² and X³ is an independently selected CR^{X}, e.g., each of X² and X³ is CH (E2).

In some embodiments, Z is —C(R^{A}R^{B})—.

In certain embodiments, Z is —CH₂—.

In some embodiments, one of R^{A} and R^{B} is a substituent other than H.

In some embodiments, m is 2 or 3.

In some embodiments,

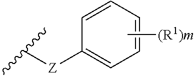

is:

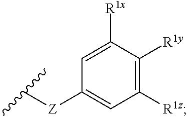

wherein:
each of R^{1x} and R^{1z} is independently selected from halogen, cyano, C1-C6 alkyl, C1-C6 haloalkyl, C1-C6 alkoxy, and C1-C6 haloalkoxy; and
R^{1y} is H or halogen (embodiment F).

In certain embodiments of (F), Rly is H (F1).

In certain embodiments of (F) and (F1), each of R^{1x} and R^{1z} is independently selected from halogen and C1-C6 haloalkyl.

In certain embodiments of (F) and (F1), each of R^{1x} and R^{1z} is independently selected from fluoro, CHF₂, and CF₃.

In certain embodiments of (F) and (F1), wherein one of R^{1x} and R^{1z} is halogen, and the other of R^{1x} and R^{1z} is C1-C6 haloalkyl.

In certain embodiments of (F) and (F1), one of R^{1x} and R^{1z} is fluoro, and the other of R^{1x} and R^{1z} is CHF₂ or CF₃.

In certain embodiments of (F) and (F1), one of R^{1x} and R^{1z} is fluoro, and the other of R^{1x} and R^{1z} is CHF₂.

In certain embodiments of (F), R^{1y} is halogen, e.g., fluoro (F2).

In certain embodiments of (F) and (F2), each of R^{1x} and R^{1z} is an independently selected halogen.

In certain embodiments of (F) and (F2), each of R^{1x} and R^{1z} is independently fluoro or chloro.

In certain embodiments of (F) and (F2), one of R^{1x} and R^{1z} is independently fluoro, and the other of R^{1x} and R^{c1z} is chloro.

In some embodiments, R^{N} is H. In some embodiments, R^{N} is a nitrogen protecting group.

In some embodiments:
Ring A is

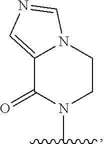 [SMILES: *N1CCn2cncc2C1=O]

which is substituted with 0-3 R² substituents independently deuterium, halogen, C1-C6 alkyl, C1-C6 deuteroalkyl, C1-C6 haloalkyl, C1-C6 alkoxy, C1-C6 deuteroalkoxy, or C1-C6 haloalkoxy;
X¹ is N or CH;
X² is CH;
X³ is N or CH;
X⁴ is CH;
Z is CH₂; and

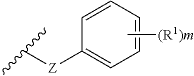

is:

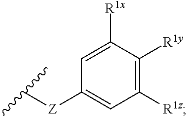

wherein:
each of R^{1x} and R^{1z} is independently selected from halogen, cyano, C1-C6 alkyl, C1-C6 haloalkyl, C1-C6 alkoxy, and C1-C6 haloalkoxy; and
R^{1y} is H or halogen (embodiment G).

In certain embodiments of (G), X¹ is N and X³ is CH; or X¹ is CH and X³ is N; or X¹ is N and X³ is N (G1).

In certain embodiments of (G) and (G1), R^{1y} is H, and one of R^{1x} and R^{1z} is halogen, and the other of R^{1x} and R^{1z} is C1-C6 haloalkyl (e.g., one of R^{1x} and R^{1z} is fluoro, and the other of R^{1x} and R^{1z} is CHF₂ or CF₃; e.g., one of R^{1x} and R^{1z} is fluoro, and the other of R^{1x} and R^{1z} is CHF₂).

In certain embodiments of (G) and (G1), R^{1y} is halogen (e.g., fluoro), and each of R^{1x} and R^{1z} is an independently selected halogen; and each of R^{1x} and R^{1z} is independently fluoro or chloro.

In some embodiments, the compound of the disclosure, e.g., a compound of Formula (J), (I), and/or (II), has the structure of Formula (IIa):

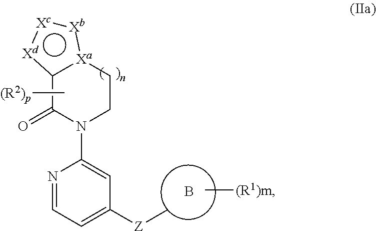

or a pharmaceutically acceptable salt thereof, wherein
X^{a} is C or N;
X^{b} is C(R²) or N(R^{N});
X^{c} and X^{d} are each independently CH, C(R²), C(O), N, or N(R^{N});
wherein when X^{a} is C, at least two of X^{b}, X^{c} and X^{d} are independently N, or N(R^{N}), and when X^{a} is N, at least one of X^{b}, X^{c} and X^{d} is N and at least one of X^{b}, X^{c} and X^{d} is C(R²);
subscript n is 1 or 2;
Z is —O—, —S—, or —C(R^{A}R^{B})—;
R^{A} and R^{B} are each independently hydrogen, halogen, or hydroxyl;
or R^{A} and R^{B}, together with the carbon atom to which they are attached, form ═O;
Ring B is phenyl or heteroaryl;
each R¹ is independently halogen, cyano, C1-C3 alkyl, C1-C3 haloalkyl, C1-C3 alkoxy, C1-C3 alkylamino, C1-C3 haloalkoxy, C3-C4 cycloalkyl, a 4-5 membered heterocyclyl having 1 heteroatom O, or SF₅, wherein the cyclohexyl and heterocyclyl are each independently substituted with 0, 1, 2 R^{1a}; or
two adjacent R¹, together with the atoms to which they are attached, form a heterocyclyl or heteroaryl ring substituted with 0, 1, or 2 R^{1a},
each R^{1a} is independently halogen, C1-C3 alkyl, or C1-C3 haloalkyl;
subscript m is 1, 2, or 3;
each R² is independently C1-C3 alkyl, C1-C3 hydroxyalkyl, C1-C3 hydroxyalkoxy, C1-C3 hydroxyalkylamino, —C(═O)—NR^{2b}R^{2c}, —(C1-C6 alkyl)-C(═O)OR^{2b}, C3-C4 cycloalkyl, or a 4-5 membered heterocyclyl having 1 heteroatom N, wherein the cycloalkyl and heterocyclyl are each independently substituted with 1 R^{2a} substituent;
R^{2a} is hydroxy or C1-C3 hydroxyalkyl;
R^{2b} and R^{2c} are each H, C1-C3 alkyl, or C1-C3 hydroxyalkyl;
or, alternatively, R^{2b} and R^{2c}, together with the nitrogen to which they are attached, form a heterocyclyl substituted with hydroxy or C1-C3 hydroxyalkyl; and
R^{N} is H, C1-C3 alkyl or C1-C3 hydroxyalkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

In some embodiments, the compound of the disclosure, e.g., a compound of Formula (J), (I), and/or (II), has the structure of Formula (IIa):

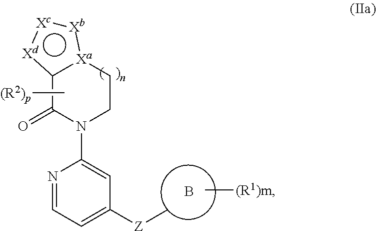

or a pharmaceutically acceptable salt thereof, wherein
X^{a} is C or N;
X^{b} is C(R²) or N(R^{N});
X^{c} and X^{d} are each independently CH, C(R²), C(O), N, or N(R^{N});
wherein when X^{a} is C, at least two of X^{b}, X^{c} and X^{d} are independently N, or N(R^{N}), and when X^{a} is N, at least one of X^{b}, X^{c} and X^{d} is N and at least one of X^{b}, X^{c} and X^{d} is C(R²);
subscript n is 1 or 2;
each R² is C1-C3 hydroxyalkyl, C1-C3 hydroxyalkoxy, C1-C3 hydroxyalkylamino, C3-C4 cycloalkyl, or a 4-5 membered heterocyclyl having 1 heteroatom N, wherein the cycloalkyl and heterocyclyl are each independently substituted with 1 R^{2a} substituent;
R^{2a} is hydroxy or C1-C3 hydroxyalkyl; and
R^{N} is C1-C3 hydroxyalkyl.

In some embodiments, the compound of the disclosure, e.g., a compound of Formula (J), (I), (II), and/or (IIa), has the structure of Formula (IIb):

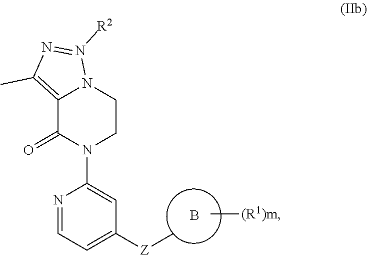

wherein
Z is —O— or —CH₂—;
R² is H, C1-C6 alkyl, C1-C6 haloalkyl, C1-C6 hydroxyalkyl, C1-C6 alkylamino, C2-C6 hydroxyalkoxy, C2-C6 hydroxyalkylamino, —S(═O)—NR^{2b}R²⁰, —C(═O)—NR^{2b}R^{2c}, —(C1-C6 alkyl)-C(═O)OR^{2b}, C3-6 cycloalkyl, or heterocyclyl, wherein the cycloalkyl and heterocyclyl are each independently substituted with 0 or 1 R^{2a},
R^{2a} is C1-C6 hydroxyalkyl;
R^{2b} and R^{2c} are each H;
Ring B is phenyl or heteroaryl;
each R¹ is independently halogen, cyano, C1-C6 alkyl, C1-C6 haloalkyl, C1-C6 alkoxy, C1-C6 alkylamino, C1-C6 haloalkoxy, C3-C6 cycloalkyl, heterocyclyl, or SF₅, wherein the cyclohexyl and heterocyclyl are each independently substituted with 0, 1, or 2 R^{1a}; or
two adjacent R¹, together with the atoms to which they are attached, form a heterocyclyl or heteroaryl ring substituted with 0, 1, or 2 R^{1a},
each R^{1a} is independently halogen, hydroxy, C1-C6 alkyl, or C1-C6 haloalkyl; and
subscript m is 1, 2, or 3;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

In some embodiments, the compound of the disclosure, e.g., a compound of Formula (J), (I), (II), and/or (IIa), has the structure of Formula (IIb):

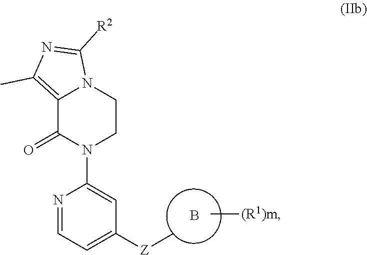

or a pharmaceutically acceptable salt thereof, wherein
R² is C1-C3 alkyl, C1-C3 hydroxyalkyl, C1-C3 hydroxyalkoxy, C1-C3 hydroxyalkylamino, C3-C4 cycloalkyl, or a 4-5 membered heterocyclyl having 1 heteroatom N, wherein the cycloalkyl and heterocyclyl are each independently substituted with 1 R^{2a} substituent; and
R^{2a} is hydroxy or C1-C3 hydroxyalkyl.

In some embodiments of the compound of Formula (IIb) or a pharmaceutically acceptable salt thereof, R² is H, C1-C3 alkyl, C1-C3 haloalkyl, C1-C3 hydroxyalkyl, C1-C3 alkylamino, C2-C3 hydroxyalkoxy, C2-C3 hydroxyalkylamino, —S(═O)—NR^{2b}R^{2c}, —C(═O)—NR^{2b}R^{2c}, —(C1-C3 alkyl)-C(═O)OR^{2b}, C3-4 cycloalkyl, or 4- to 5-membered heterocyclyl, wherein the cycloalkyl and heterocyclyl are each independently substituted with 0 or 1 R^{2a} In some embodiments, R² is H, CH₃, —CH₂OH, —CH₂CH₂OH, —CH₂CH₂CH₂OH, —CH(OH)CH₂OH, —OCH₂CH₂OH, —NHCH₂CH₂OH, —S(O)NH₂, —C(O)NH₂, —CH₂C(O)OH,

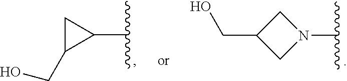 [SMILES: *C1CC1CO ; *N1CC(CO)C1]

In some embodiments of the compound of Formula (IIb) or a pharmaceutically acceptable salt thereof, Ring B is

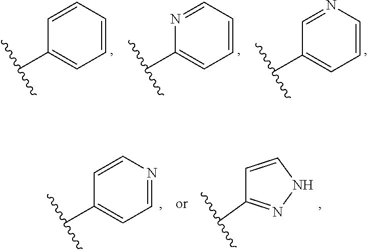

which are each substituted by (R¹)ₘ.

In some embodiments of the compound of Formula (IIb) or a pharmaceutically acceptable salt thereof, each R¹ is independently halogen, cyano, C1-C3 alkyl, C1-C3 haloalkyl, C1-C3 alkoxy, C1-C3 alkylamino, C1-C3 haloalkoxy, C3-C4 cycloalkyl, 4- to 5-membered heterocyclyl, or SF₅, wherein the cyclohexyl and heterocyclyl are each independently substituted with 0, 1, or 2 R^{1a}. In some embodiments, each R¹ is independently F, Cl, cyano, CH₃, CF₂H, CF₃, CF₂CH₃, OCH₃, NHCH₃, NHCH₂CH₃, OCF₃, cyclopropyl, oxetanyl, or SF₅, wherein the cyclopropyl is substituted with 0, 1, 2 F or CH₃.

In some embodiments of the compound of Formula (IIb) or a pharmaceutically acceptable salt thereof, two adjacent R¹, together with the atoms to which they are attached, form a tetrahydrofuranyl, tetrahydropyranyl, 2-oxabicyclo[3.1.0]hexyl, morpholinyl, pyrazolyl, isoxazolyl, or pyridyl ring substituted with 0, 1 or 2 R^{1a}, and each R^{1a} is independently F, hydroxy, CH₃, or CF₃.

In some embodiments of the compound of Formula (IIb) or a pharmaceutically acceptable salt thereof, Z is —O— or —CH₂—; R² is H, CH₃, —CH₂OH, —CH₂CH₂OH, —CH₂CH₂CH₂OH, —CH(OH)CH₂OH, —OCH₂CH₂OH, —NHCH₂CH₂OH, —S(O)NH₂, —C(O)NH₂, —CH₂C(O)OH,

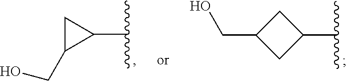 [SMILES: *C1CC1CO ; *C1CC(CO)C1]

Ring B is

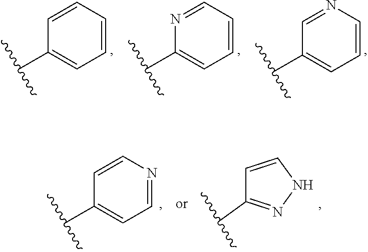

which are each substituted by (R¹)ₘ; each R¹ is independently F, Cl, cyano, CH₃, CF₂H, CF₃, CF₂CH₃, OCH₃, NHCH₃, NHCH₂CH₃, OCF₃, cyclopropyl, oxetanyl, or SF₅, wherein the cyclopropyl is substituted with 0, 1, 2 F or CH₃; or two adjacent R¹, together with the atoms to which they are attached, form a tetrahydrofuranyl, tetrahydropyranyl, 2-oxabicyclo[3.1.0]hexyl, morpholinyl, pyrazolyl, isoxazolyl, or pyridyl ring substituted with 0, 1 or 2 R^{1a}; each R^{1a} is independently F, hydroxy, CH₃, or CF₃; and subscript m is 1, 2, or 3.

In some embodiments of the compound of Formula (IIb) or a pharmaceutically acceptable salt thereof, Z is —O— or —CH₂—; R² is —CH₂OH, —CH₂CH₂OH, —CH₂CH₂CH₂OH, —CH(OH)CH₂OH, —OCH₂CH₂OH, —NHCH₂CH₂OH,

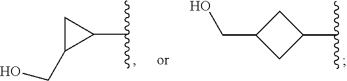 [SMILES: *C1CC1CO ; *C1CC(CO)C1]

### Ring B is

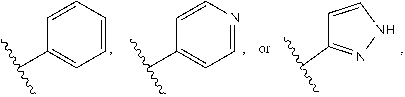

which are each substituted by (R¹)ₘ; each R¹ is independently F, Cl, cyano, CH₃, CF₂H, CF₃, CF₂CH₃, OCH₃, NHCH₃, NHCH₂CH₃, OCF₃, cyclopropyl, oxetanyl, or SFs, wherein the cyclopropyl is substituted with 0, 1, 2 F or CH₃; or two adjacent R¹, together with the atoms to which they are attached, form a tetrahydrofuranyl, tetrahydropyranyl, 2-oxabicyclo[3.1.0]hexyl, morpholinyl, pyrazolyl, isoxazolyl, or pyridyl ring substituted with 0, 1 or 2 R^{1a}; each R^{1a} is independently F, hydroxy, CH₃, or CF₃, and subscript m is 1, 2, or 3.

In some embodiments of the compound of Formula (IIb) or a pharmaceutically acceptable salt thereof,

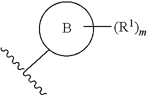

is

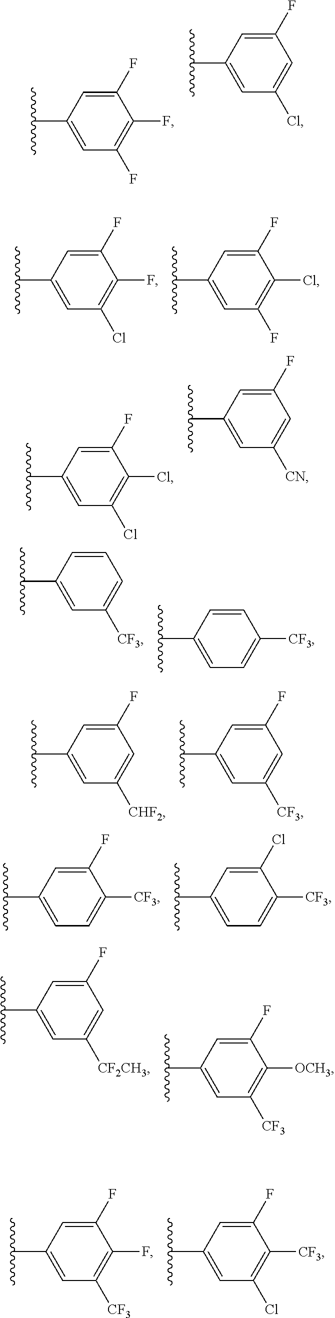

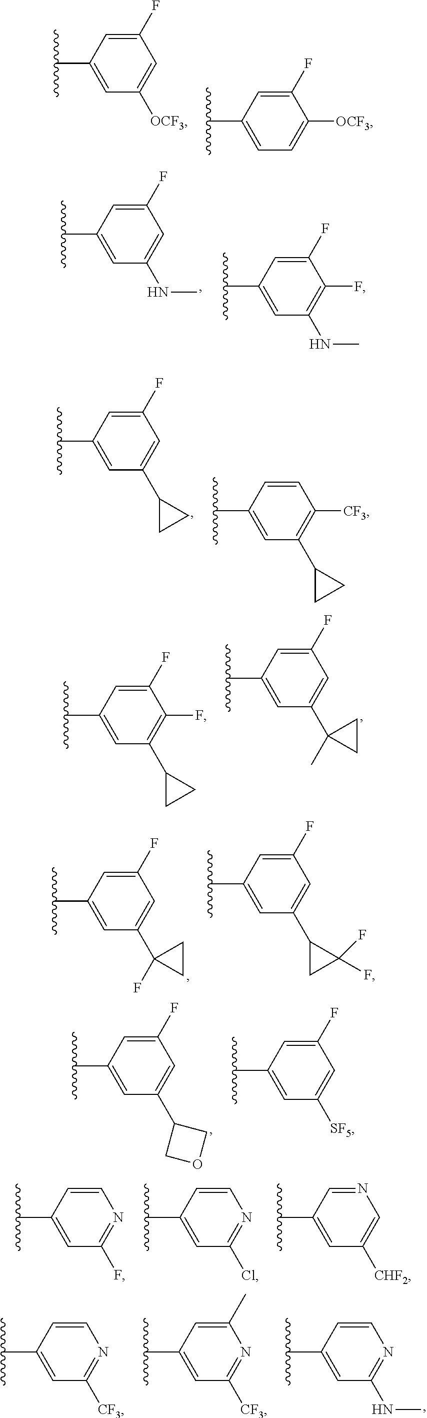

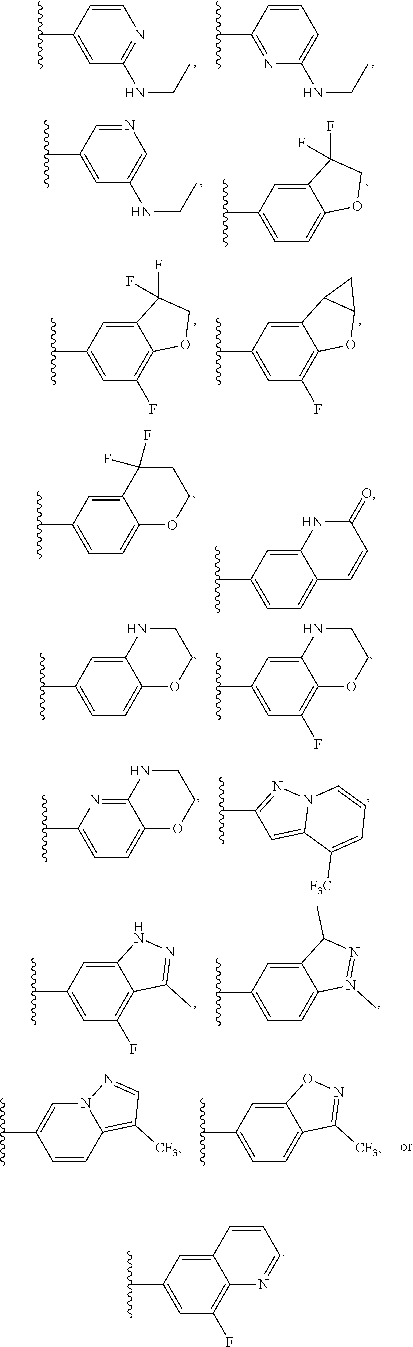

In some embodiments of the compound of Formula (IIb) or a pharmaceutically acceptable salt thereof,

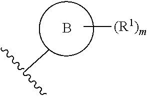

is

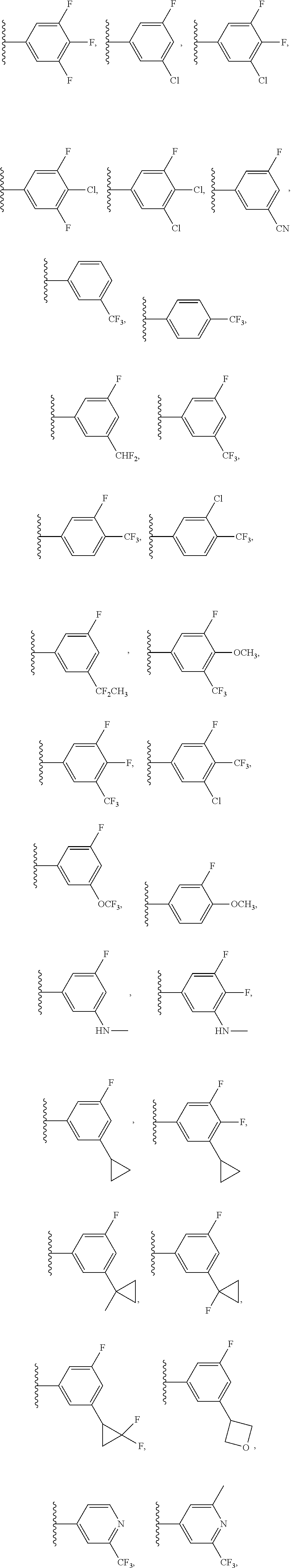

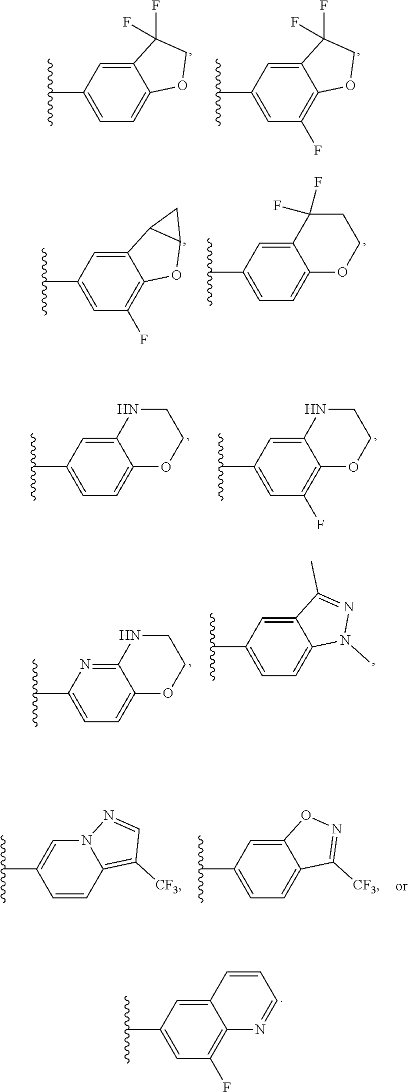

In some embodiments, the compound of the disclosure, e.g., a compound of Formula (J), (I), (II), and/or (IIa), has the structure of Formula (IIc):

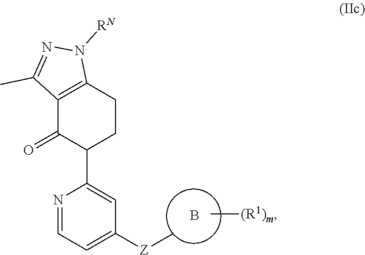

or a pharmaceutically acceptable salt thereof, wherein
R^{N} is C1-C3 alkyl or C1-C3 hydroxyalkyl.

In some embodiments, the compound of the disclosure, e.g., a compound of Formula (J), (I), and/or (II), has the structure of Formula (IId):

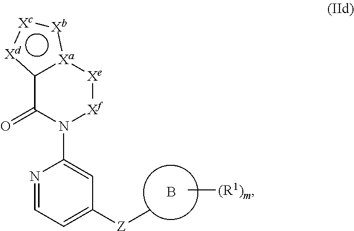

or a pharmaceutically acceptable salt thereof, wherein
X^{a} is C or N;
X^{b} is C(R²) or N(R^{N});
X^{c} and X^{d} are each independently CH, C(R²), C(O), N, or N(R^{N});
X^{e} and X^{f} are each independently CH, C(R²), or N;
wherein when X^{a} is C, at least two of X^{b}, X^{c} and X^{d} are independently N, or N(R^{N}), and
when X^{a} is N, at least one of X^{b}, X^{c} and X^{d} is N and at least one of X^{b}, X^{c} and X^{d} is C(R²);
Z is —O—, —S—, or —C(R^{A}R^{B})—; R^{A} and R^{B} are each independently hydrogen, halogen, or hydroxyl;
or R^{A} and R^{B}, together with the carbon atom to which they are attached, form ═O;
Ring B is phenyl or heteroaryl;
each R¹ is independently halogen, cyano, C1-C3 alkyl, C1-C3 haloalkyl, C1-C3 alkoxy, C1-C3 alkylamino, C1-C3 haloalkoxy, C3-C4 cycloalkyl, a 4-5 membered heterocyclyl having 1 heteroatom O, or SF₅, wherein the cyclohexyl and heterocyclyl are each independently substituted with 0, 1, 2 R^{1a}; or
two adjacent R¹, together with the atoms to which they are attached, form a heterocyclyl or heteroaryl ring substituted with 0, 1, or 2 R^{1a},
each R^{1a} is independently halogen, C1-C3 alkyl, or C1-C3 haloalkyl;
subscript m is 1, 2, or 3;
each R² is amino, C1-C3 hydroxyalkyl, or C1-C3 hydroxyalkylamino; and
R^{N} is C1-C3 alkyl or C1-C3 hydroxyalkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

In some embodiments, the compound of the disclosure, e.g., a compound of Formula (J), (I), and/or (II), has the structure of Formula (IId):

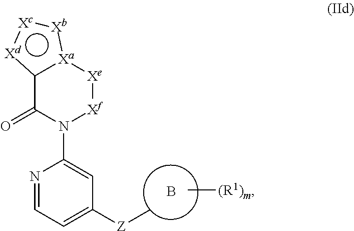

or a pharmaceutically acceptable salt thereof, wherein
X^{a} is C or N;
X^{b} is C(R²) or N(R^{N});
X^{c} and X^{d} are each independently CH, C(R²), C(O), N, or N(R^{N});
wherein when X^{a} is C, at least two of X^{b}, X^{c} and X^{d} are independently N, or N(R^{N}), and when X^{a} is N, at least one of X^{b}, X^{c} and X^{d} is N and at least one of X^{b}, X^{c} and X^{d} is C(R²);
each R² is amino, C1-C3 hydroxyalkyl, or C1-C3 hydroxyalkylamino; and
R^{N} is C1-C3 alkyl or C1-C3 hydroxyalkyl.

In some embodiments, the compound of the disclosure, e.g., a compound of Formula (J), (I), and/or (II), has the structure of Formula (IIe):

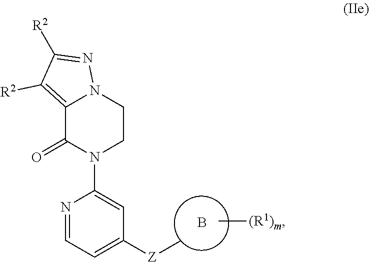

or a pharmaceutically acceptable salt thereof, wherein
Z is —O—, —S—, or —C(R^{A}R^{B})—;
R^{A} and R^{B} are each independently hydrogen, halogen, or hydroxyl; or R^{A} and R^{B}, together with the carbon atom to which they are attached, form ═O;
Ring B is phenyl or heteroaryl;
each R¹ is independently halogen, cyano, C1-C3 alkyl, C1-C3 haloalkyl, C1-C3 alkoxy, C1-C3 alkylamino, C1-C3 haloalkoxy, C3-C4 cycloalkyl, a 4-5 membered heterocyclyl having 1 heteroatom O, or SF₅, wherein the cyclohexyl and heterocyclyl are each independently substituted with 0, 1, 2 R^{1a}, or
two adjacent R¹, together with the atoms to which they are attached, form a heterocyclyl or heteroaryl ring substituted with 0, 1, or 2 R^{1a},
each R^{1a} is independently halogen, C1-C3 alkyl, or C1-C3 haloalkyl;
subscript m is 1, 2, or 3;
each R² is independently H, C1-C3 alkyl, C1-C3 hydroxyalkyl, C1-C3 hydroxyalkoxy, C1-C3 hydroxyalkylamino, —C(═O)—NR²⁶R^{2c}, —(C1-C6 alkyl)-C(═O)OR^{2b}, C3-C4 cycloalkyl, or a 4-5 membered heterocyclyl having 1 heteroatom N, wherein the cycloalkyl and heterocyclyl are each independently substituted with 1 R^{2a} substituent; and
R^{2a} is hydroxy or C1-C3 hydroxyalkyl; and
R^{2b} and R^{2c} are each H, C1-C3 alkyl, or C1-C3 hydroxyalkyl;
or, alternatively, R^{2b} and R^{2c}, together with the nitrogen to which they are attached, form a heterocyclyl substituted with hydroxy or C1-C3 hydroxyalkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

In some embodiments of the compound of Formula (IIe) or a pharmaceutically acceptable salt thereof, each R² is independently H, C1-C3 alkyl, C1-C3 hydroxyalkyl, C1-C3 alkylamino, C1-C3 hydroxyalkoxy, —C(═O)—NR^{2b}R^{2c}; R^{2b} and R^{2c} are each H, C1-C3 alkyl, or C1-C3 hydroxyalkyl; or, alternatively, R^{2b} and R^{2c}, together with the nitrogen to which they are attached, form a 4- to 5-membered heterocyclyl substituted with hydroxy or C1-C3 hydroxyalkyl. In some embodiments, each R² is independently H, CH₃, CH₂CH₃, —CH₂OH, —CH₂CH₂OH, —OCH₂CH₂OH, —CH₂NH₂, —NHCH₃, —NHCH₂CH₃, —C(O)NH₂, —(C═O)NHCH₂CH₂OH, —(C═O)N(CH₃) CH₂CH₂OH,

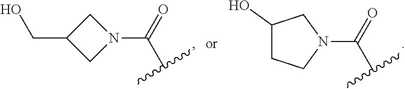 [SMILES: *C(=O)N1CC(CO)C1 ; *C(=O)N1CCC(O)C1]

In some embodiments of the compound of Formula (IIe) or a pharmaceutically acceptable salt thereof, each R¹ is independently halogen, C1-C3 haloalkyl, or C1-C3 haloalkoxy. In some embodiments, each R¹ is independently F, Cl, CF₃, or OCF₃; or two adjacent R¹, together with the atoms to which they are attached, form a tetrahydrofuranyl or morpholinyl ring substituted with 0, 1 or 2 R^{1a}, and R^{1a} is F.

In some embodiments of the compound of Formula (IIe) or a pharmaceutically acceptable salt thereof, Z is —O— or —CH₂—; each R² is independently H, CH₃, CH₂CH₃, —CH₂OH, —CH₂CH₂OH, —OCH₂CH₂OH, —CH₂NH₂, —NHCH₃, —NHCH₂CH₃, —C(O)NH₂, —(C═O)NHCH₂CH₂OH, —(C═O)N(CH₃) CH₂CH₂OH,

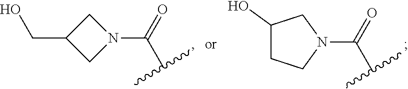 [SMILES: *C(=O)N1CC(CO)C1 ; *C(=O)N1CCC(O)C1]

### Ring B is

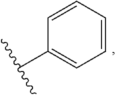 [SMILES: *c1ccccc1]

which is substituted by (R¹)ₘ; each R¹ is independently F, Cl, CF₃, or OCF₃; or two adjacent R¹, together with the atoms to which they are attached, form a tetrahydrofuranyl or morpholinyl ring substituted with 0, 1 or 2 R^{1a}; R^{1a} is F; and subscript m is 1, 2, or 3.

In some embodiments of the compound of Formula (IIe) or a pharmaceutically acceptable salt thereof,

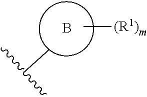

is

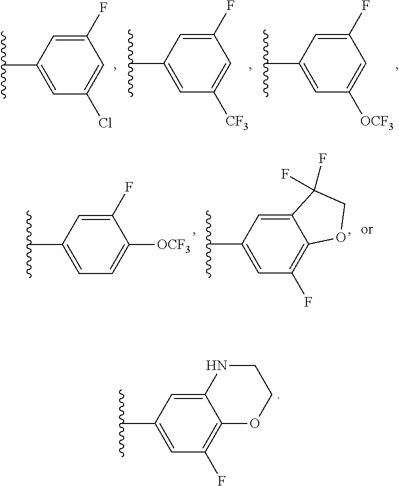

In some embodiments, the compound of the disclosure, e.g., a compound of Formula (J), (I), and/or (II), has the structure of Formula (IIf):

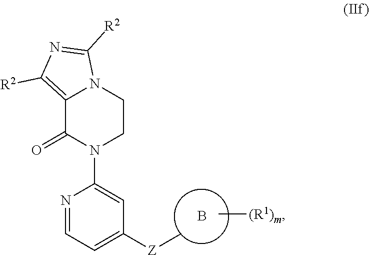

or a pharmaceutically acceptable salt thereof, wherein
Z is —O—, —S—, or —C(R^{A}R^{B})—;
R^{A} and R^{B} are each independently hydrogen, halogen, or hydroxyl;
or R^{A} and R^{B}, together with the carbon atom to which they are attached, form ═O; Ring B is phenyl or heteroaryl;
each R¹ is independently halogen, cyano, C1-C3 alkyl, C1-C3 haloalkyl, C1-C3 alkoxy, C1-C3 alkylamino, C1-C3 haloalkoxy, C3-C4 cycloalkyl, a 4-5 membered heterocyclyl having 1 heteroatom O, or SF₅, wherein the cyclohexyl and heterocyclyl are each independently substituted with 0, 1, 2 R^{1a}, or
two adjacent R¹, together with the atoms to which they are attached, form a heterocyclyl or heteroaryl ring substituted with 0, 1, or 2 R^{1a},
each R^{1a} is independently halogen, C1-C3 alkyl, or C1-C3 haloalkyl;
subscript m is 1, 2, or 3;
each R² is independently H, C1-C3 alkyl, C1-C3 hydroxyalkyl, C1-C3 hydroxyalkoxy, C1-C3 hydroxyalkylamino, —S(═O)—NR^{2b}R^{2c}, —C(═O)—NR^{2b}R^{2c}, —(C1-C6 alkyl)-C(═O)OR^{2b}, C3-C4 cycloalkyl, or a 4-5 membered heterocyclyl having 1 heteroatom N, wherein the cycloalkyl and heterocyclyl are each independently substituted with 1 R^{2a} substituent; and
R^{2a} is hydroxy or C1-C3 hydroxyalkyl; and
R^{2b} and R^{2c} are each H, C1-C3 alkyl, or C1-C3 hydroxyalkyl;
or, alternatively, R^{2b} and R^{2c}, together with the nitrogen to which they are attached, form a heterocyclyl substituted with hydroxy or C1-C3 hydroxyalkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

In some embodiments, the compound of the disclosure, e.g., a compound of Formula (J), (I), and/or (II), has the structure of Formula (IIg):

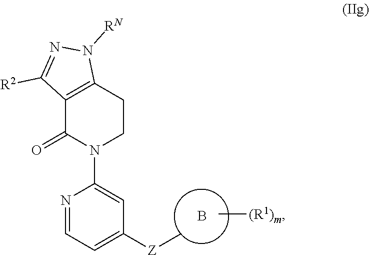

or a pharmaceutically acceptable salt thereof, wherein
Z is —O—, —S—, or —C(R^{A}R^{B})—;
R^{A} and R^{B} are each independently hydrogen, halogen, or hydroxyl;
or R^{A} and R^{B}, together with the carbon atom to which they are attached, form ═O; Ring B is phenyl or heteroaryl;
each R¹ is independently halogen, cyano, C1-C3 alkyl, C1-C3 haloalkyl, C1-C3 alkoxy, C1-C3 alkylamino, C1-C3 haloalkoxy, C3-C4 cycloalkyl, a 4-5 membered heterocyclyl having 1 heteroatom O, or SF₅, wherein the cyclohexyl and heterocyclyl are each independently substituted with 0, 1, 2 R^{1a}; or
two adjacent R¹, together with the atoms to which they are attached, form a heterocyclyl or heteroaryl ring substituted with 0, 1, or 2 R^{1a},
each R^{1a} is independently halogen, C1-C3 alkyl, or C1-C3 haloalkyl;
subscript m is 1, 2, or 3; and
R² is C1-C3 alkyl, C1-C3 hydroxyalkyl, or (benzyloxy)(C1-C6 alkyl);
R^{N} is C1-C3 alkyl, C1-C3 hydroxyalkyl, or (benzyloxy)(C1-C6 alkyl);
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

In some embodiments of the compound of Formula (IIg) or a pharmaceutically acceptable salt thereof, each R¹ is independently halogen, cyano, C1-C3 haloalkyl, C1-C3 haloalkoxy, C3-C4 cycloalkyl, or 4- to 5-membered heterocyclyl, wherein the cyclohexyl and heterocyclyl are each independently substituted with 0, 1, 2 R^{1a}.

In some embodiments of the compound of Formula (IIg) or a pharmaceutically acceptable salt thereof, each R^{1a} is independently halogen, C1-C3 alkyl, or C1-C3 haloalkyl.

In some embodiments of the compound of Formula (IIg) or a pharmaceutically acceptable salt thereof, R² is C1-C3 alkyl, C1-C3 hydroxyalkyl, or (benzyloxy)(C1-C3 alkyl). In some embodiments, R² is CH₃, CH₂CH₃, —CH₂OH, or —CH₂OBn.

In some embodiments of the compound of Formula (IIg) or a pharmaceutically acceptable salt thereof, Ring B is

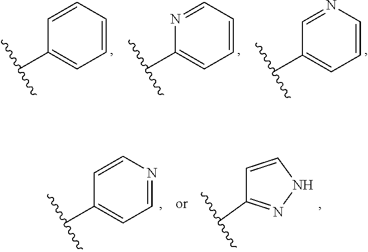

which are each substituted by (R¹)ₘ.

In some embodiments of the compound of Formula (IIg) or a pharmaceutically acceptable salt thereof, each R¹ is independently F, Cl, cyano, CH₃, CF₂H, CF₃, OCF₃, cyclopropyl, oxetanyl, wherein the cyclopropyl is substituted with 0, 1, 2 R^{1a}; or two adjacent R¹, together with the atoms to which they are attached, form a tetrahydrofuranyl, dihydropyranyl, tetrahydropyranyl, 2-oxabicyclo[3.1.0]hexyl, morpholinyl, pyrazolyl, isoxazolyl, or pyridyl ring substituted with 0, 1 or 2 R^{1a}; and each R^{1a} is independently F, CH₃, or CF₃.

In some embodiments of the compound of Formula (IIg) or a pharmaceutically acceptable salt thereof, R^{N} is H, C1-C3 alkyl, C1-C3 hydroxyalkyl, or (benzyloxy)(C1-C3 alkyl). In some embodiments, R^{N} is H, CH₃, —CH₂CH₂OH, or —CH₂CH₂OBn.

In some embodiments of the compound of the disclosure, e.g., a compound of Formula (J), (I), (II), (IIa), (IIb), (IIc), (IId), (IIe), (IIf), and/or (IIg), each R² is independently CH₃, CH₂CH₃, CF₃, CH₂OH, CH₂CH₂OH, CH(OH)CH₂OH, CH₂CH₂CH₂OH, OCH₃, OCH₂CH₃, OCH₂CH₂OH, CH₂NH₂, NH₂, NHCH₃, NHCH₂CH₃, NHCH₂CH₂OH, —S(═O)—NH₂, —(C═O)—NH₂, —(C═O)NHCH₂CH₂OH, —(C═O)N(CH₃) CH₂CH₂OH, —CH₂ (C═O)OH,

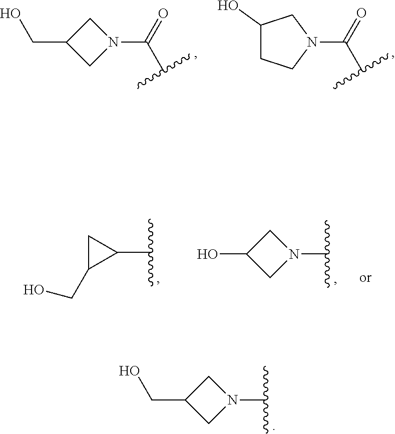 [SMILES: *C(=O)N1CC(CO)C1 ; *C(=O)N1CCC(O)C1 ; *C1CC1CO ; *N1CC(O)C1 ; *N1CC(CO)C1]

In some embodiments, R² is CH₂OH, CH₂CH₂OH, CH(OH)CH₂OH, CH₂CH₂CH₂OH, OCH₂CH₂OH, NHCH₂CH₂OH, —(C═O)NHCH₂CH₂OH, —(C═O)N(CH₃) CH₂CH₂OH,

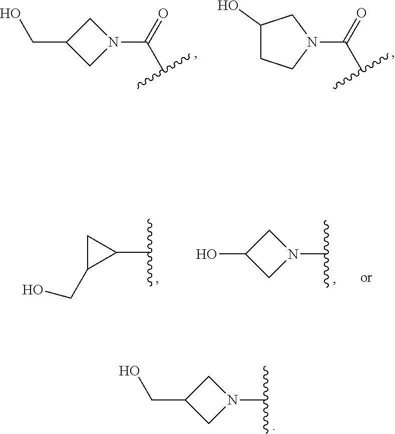 [SMILES: *C(=O)N1CC(CO)C1 ; *C(=O)N1CCC(O)C1 ; *C1CC1CO ; *N1CC(O)C1 ; *N1CC(CO)C1]

In some embodiments, R² is CH₃, CH₂CH₃, CH₂OH, CH₂CH₂OH, CH₂CH₂CH₂OH, OCH₃, OCH₂CH₂OH, NH₂, NHCH₃, NHCH₂CH₂OH, —(C═O)—NH₂,

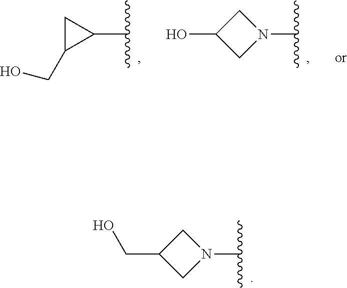 [SMILES: *C1CC1CO ; *N1CC(O)C1 ; *N1CC(CO)C1]

In some embodiments,
R² is CH₂OH, CH₂CH₂OH, CH₂CH₂CH₂OH, OCH₂CH₂OH, NHCH₂CH₂OH,

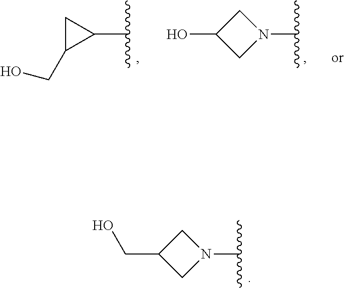 [SMILES: *C1CC1CO ; *N1CC(O)C1 ; *N1CC(CO)C1]

In some embodiments of the compound of the disclosure, e.g., a compound of Formula (J), (I), (II), (IIa), (IIb), (IIc), (IId), (IIe), (IIf), and/or (IIg), R^{N} is H, CH₃, CH₂CH₃, or CH₂CH₂OH. In some embodiments, R^{N} is CH₂CH₂OH.

In some embodiments, Ring B is pyrazolyl, thiazolyl, phenyl, or pyridyl.

In some embodiments of the compound of the disclosure, e.g., a compound of Formula (J), (I), (II), (IIa), (IIb), (IIc), (IId), (IIe), (IIf), and/or (IIg), each R¹ is independently F, Cl, CN, OCH₃, CHF₂, CF₃, NHCH₃, NHCH₂CH₃, OCF₃, cyclopropyl, or

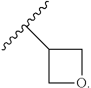 [SMILES: *C1COC1]

In some embodiments, each R^{1a} is independently F, OH, CH₃, or CF₃. In some embodiments, R^{1a} is CH₃.

In some embodiments of the compound of the disclosure, e.g., a compound of Formula (J), (I), (II), (IIa), (IIb), (IIc), (IId), (IIe), (IIf), and/or (IIg), the moiety

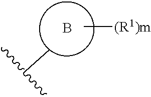

is

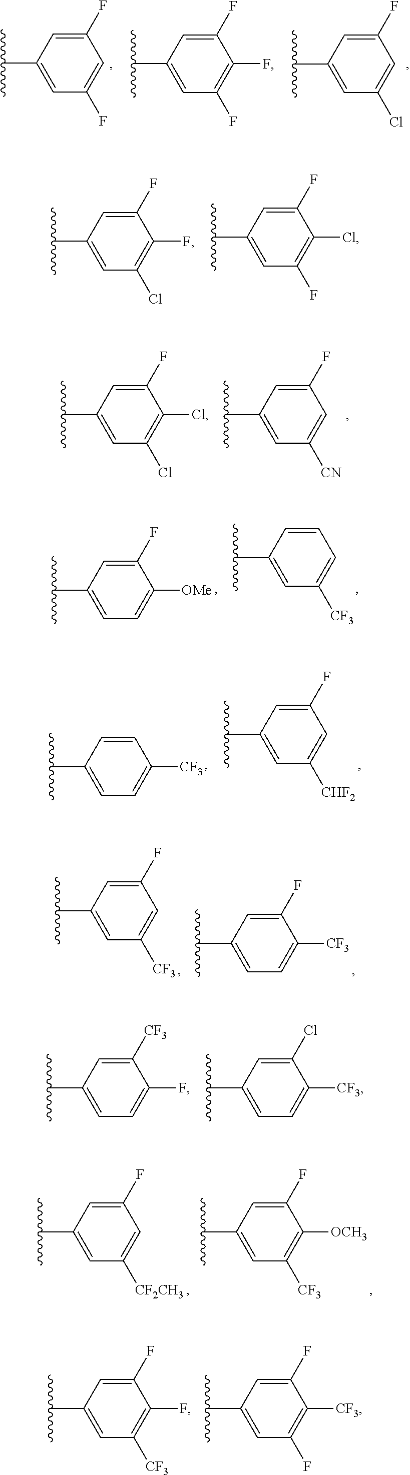

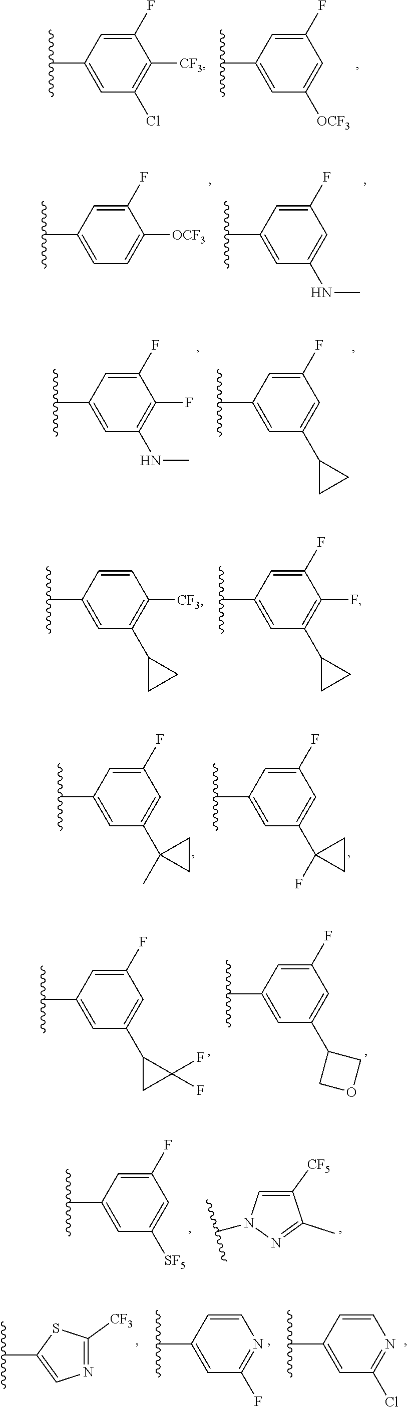

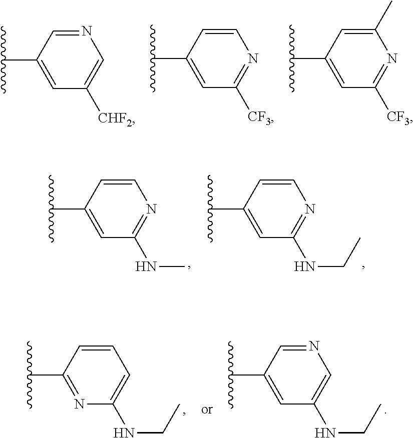

In some embodiments, the moiety

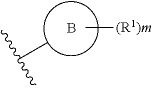

is

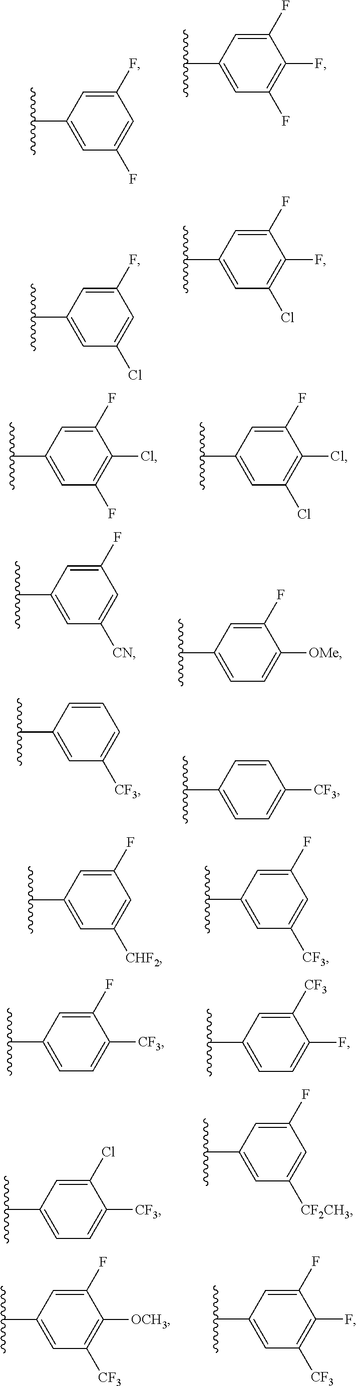

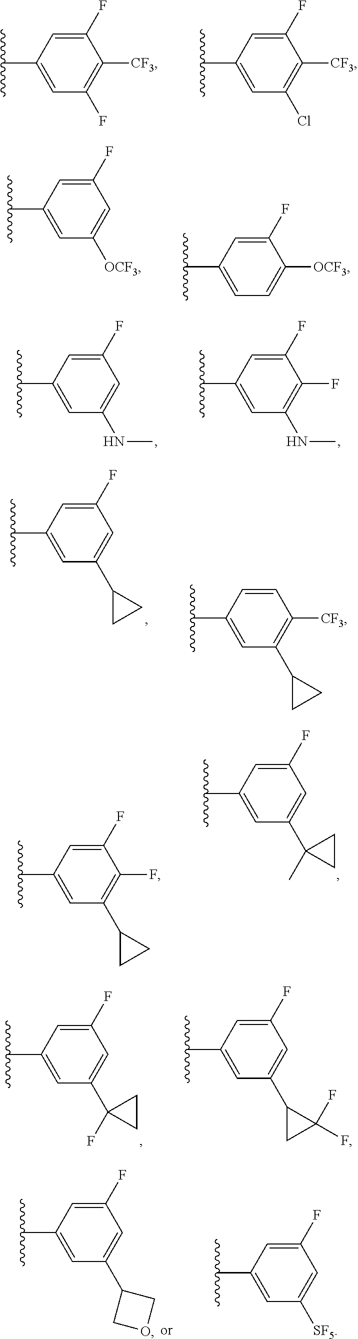

In some embodiments, the moiety

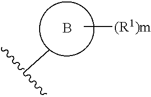

is

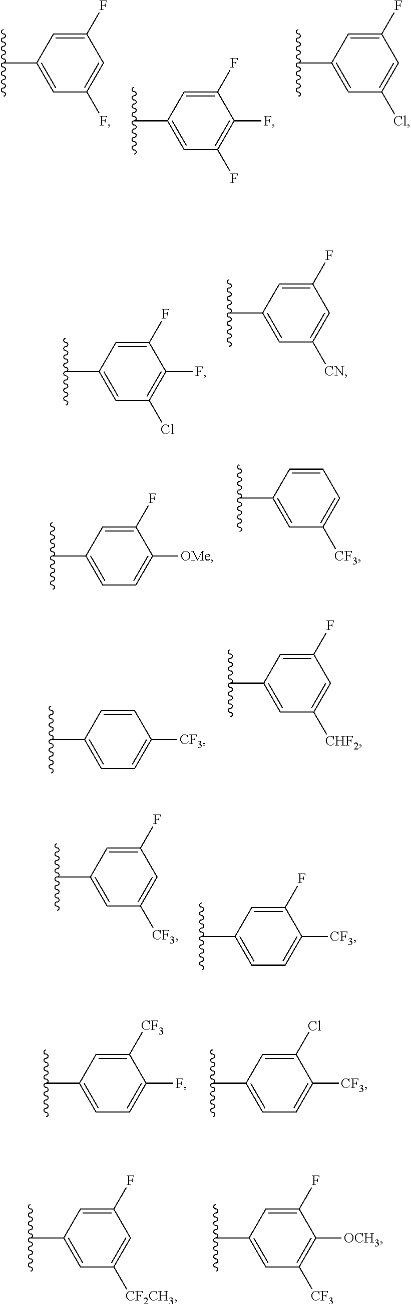

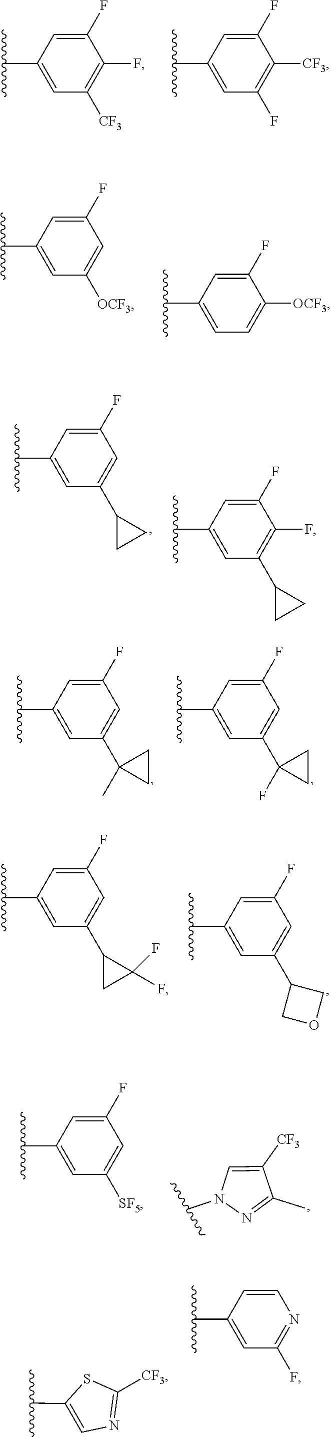

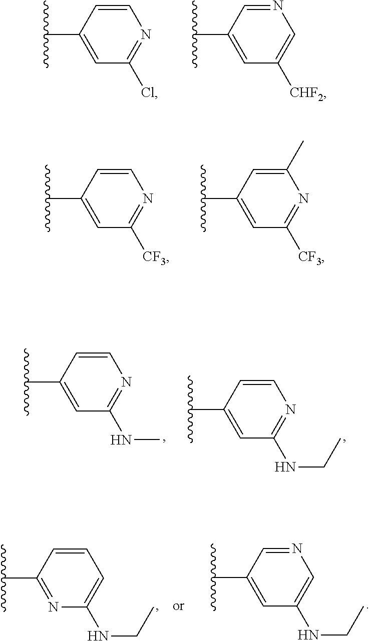

In some embodiments, the moiety

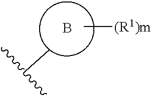

is

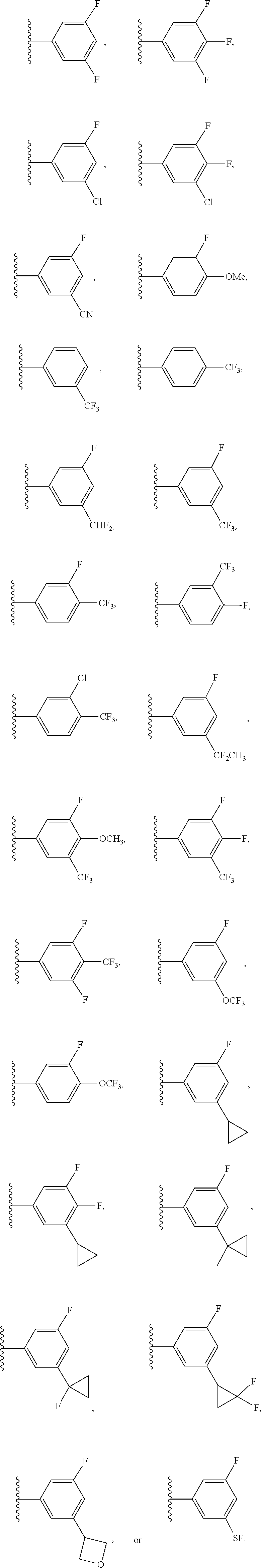

In some embodiments, the moiety

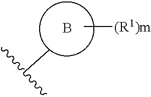

is

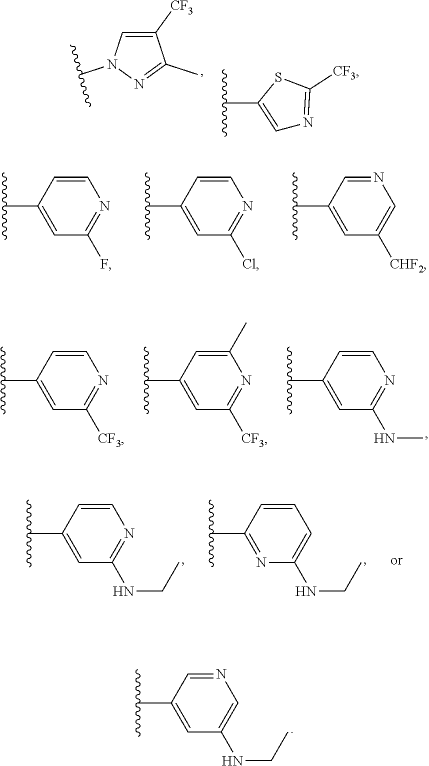

In some embodiments, the moiety

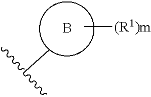

is

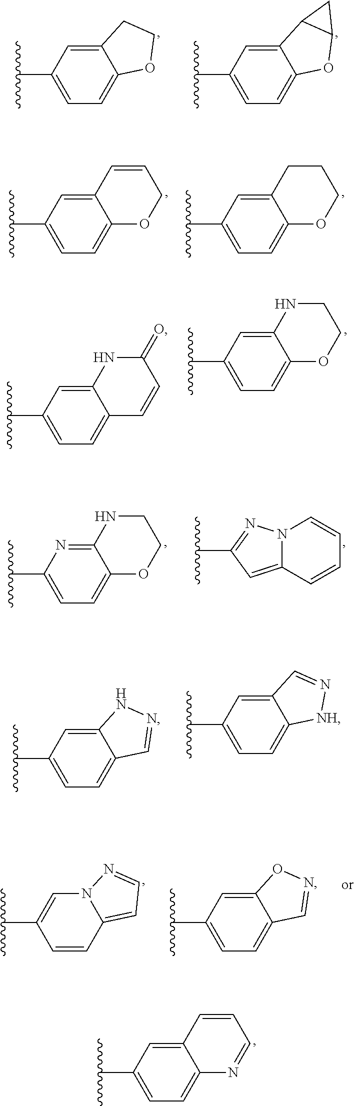

which are each substituted with 0-3 R^{1a}.

In some embodiments, R^{1a} is F, OH, CH₃, or CF₃.

In some embodiments, the moiety

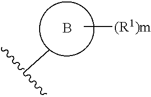

is

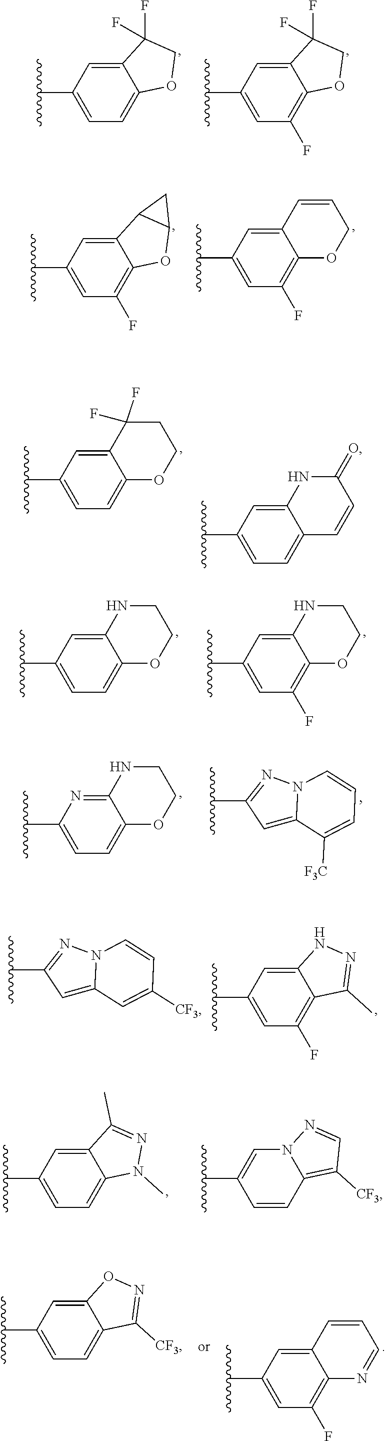

In some embodiments, the moiety

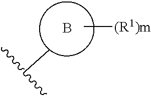

is

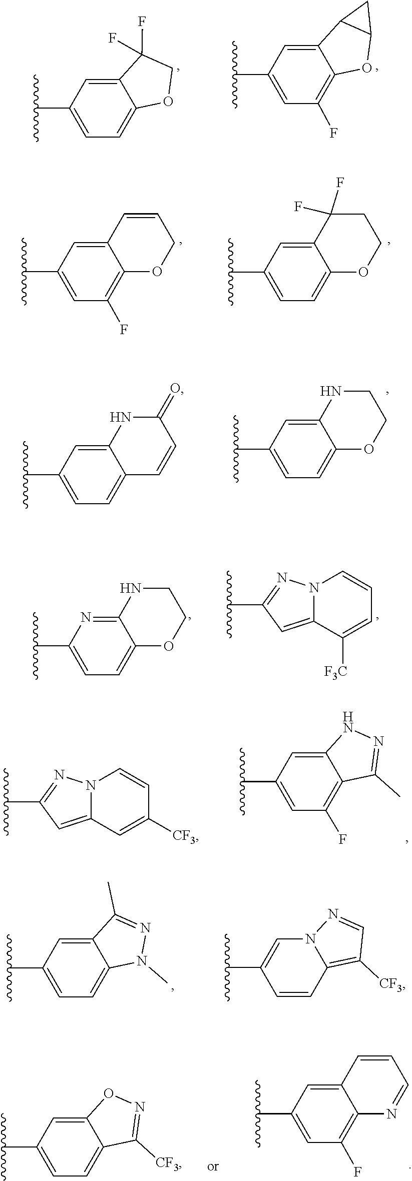

In some embodiments, Z is —O—, —S—, —CH₂—, or —C(═O)—.

In some embodiments, Z is —O— or —CH₂—; Ring B is phenyl; each R¹ is independently F, Cl, cyano, CH₃, CF₂H, CF₃, OCH₃, NHCH₃, NHCH₂CH₃, OCF₃, cyclopropyl, oxetanyl, or SF₅, wherein the cyclopropyl is substituted with 0, 1, 2 F or CH₃; or two adjacent R¹, together with the atoms to which they are attached, form a tetrahydrofuranyl, tetrahydropyranyl, morpholinyl, pyrazolyl, isoxazolyl, or pyridyl ring substituted with 0, 1 or 2 R^{1a}; each R^{1a} is independently F, CH₃, or CF₃; subscript m is 1, 2, or 3; each R² is independently CH₃, CH₂CH₃, CF₃, CH₂OH, CH₂CH₂OH, CH(OH)CH₂OH, CH₂CH₂CH₂OH, OCH₃, OCH₂CH₃, OCH₂CH₂OH, CH₂NH₂, NH₂, NHCH₃, NHCH₂CH₃, NHCH₂CH₂OH, —(C═O)—NH₂, —(C═O)NHCH₂CH₂OH, —(C—O)N(CH₃) CH₂CH₂OH, —CH₂ (C═O)OH,

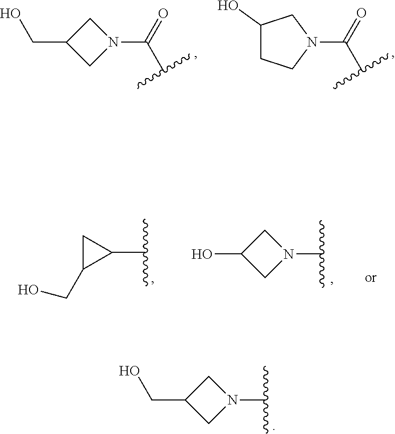 [SMILES: *C(=O)N1CC(CO)C1 ; *C(=O)N1CCC(O)C1 ; *C1CC1CO ; *N1CC(O)C1 ; *N1CC(CO)C1]

In some embodiments of the compound of the disclosure, e.g., a compound of Formula (J), (I), (II), (IIa), (IIb), (IIc), (IId), (IIe), (IIf), and/or (IIg), the compound of the disclosure has the structure of a compound in Table 1.

TABLE 1
Compounds

| Compound | |
|---|---|
| No. | Structure |
| 1 | 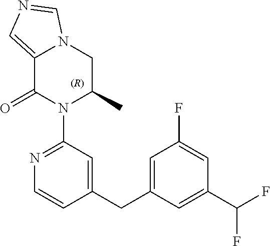 |
| 2 | 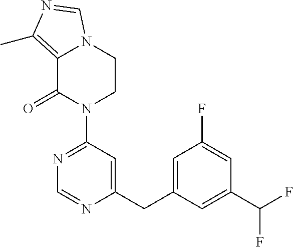 [SMILES: Cc1ncn2c1C(=O)N(c1cc(Cc3cc(F)cc(C(F)F)c3)ncn1)CC2] |
| 3 | 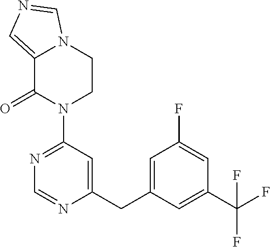 [SMILES: O=C1c2cncn2CCN1c1cc(Cc2cc(F)cc(C(F)(F)F)c2)ncn1] |
| 4 | 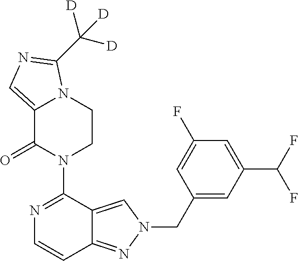 [SMILES: [2H]C([2H])([2H])c1ncc2n1CCN(c1nccc3nn(Cc4cc(F)cc(C(F)F)c4)cc13)C2=O] |
| 5 | 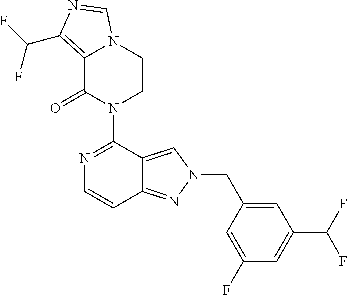 [SMILES: O=C1c2c(C(F)F)ncn2CCN1c1nccc2nn(Cc3cc(F)cc(C(F)F)c3)cc12] |
| 6 | 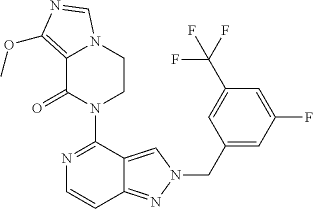 [SMILES: COc1ncn2c1C(=O)N(c1nccc3nn(Cc4cc(F)cc(C(F)(F)F)c4)cc13)CC2] |
| 7 | 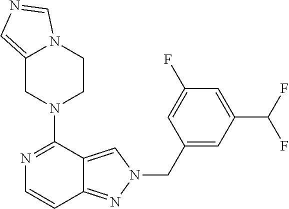 [SMILES: Fc1cc(Cn2cc3c(N4CCn5cncc5C4)nccc3n2)cc(C(F)F)c1] |
| 8 | 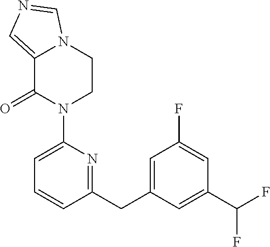 [SMILES: O=C1c2cncn2CCN1c1cccc(Cc2cc(F)cc(C(F)F)c2)n1] |
| 9 | 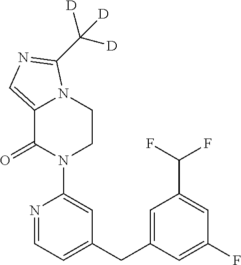 [SMILES: [2H]C([2H])([2H])c1ncc2n1CCN(c1cc(Cc3cc(F)cc(C(F)F)c3)ccn1)C2=O] |
| 10 | 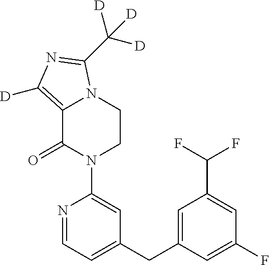 [SMILES: [2H]c1nc(C([2H])([2H])[2H])n2c1C(=O)N(c1cc(Cc3cc(F)cc(C(F)F)c3)ccn1)CC2] |
| 11 | 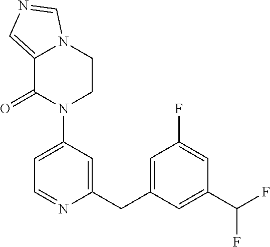 [SMILES: O=C1c2cncn2CCN1c1ccnc(Cc2cc(F)cc(C(F)F)c2)c1] |
| 12 | 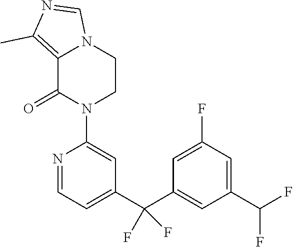 [SMILES: Cc1ncn2c1C(=O)N(c1cc(C(F)(F)c3cc(F)cc(C(F)F)c3)ccn1)CC2] |
| 13 | 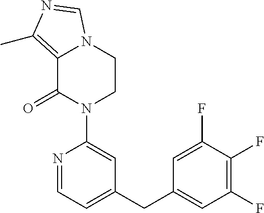 [SMILES: Cc1ncn2c1C(=O)N(c1cc(Cc3cc(F)c(F)c(F)c3)ccn1)CC2] |
| 14 | 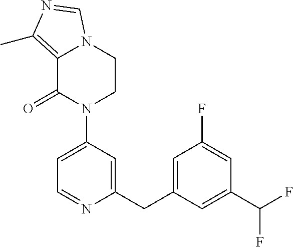 [SMILES: Cc1ncn2c1C(=O)N(c1ccnc(Cc3cc(F)cc(C(F)F)c3)c1)CC2] |
| 15 | 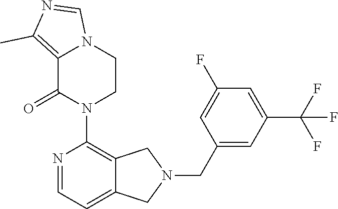 [SMILES: Cc1ncn2c1C(=O)N(c1nccc3c1CN(Cc1cc(F)cc(C(F)(F)F)c1)C3)CC2] |
| 16 | 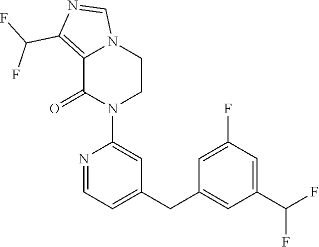 [SMILES: O=C1c2c(C(F)F)ncn2CCN1c1cc(Cc2cc(F)cc(C(F)F)c2)ccn1] |
| 17 | 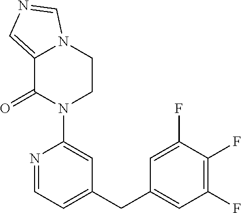 [SMILES: O=C1c2cncn2CCN1c1cc(Cc2cc(F)c(F)c(F)c2)ccn1] |
| 18 | 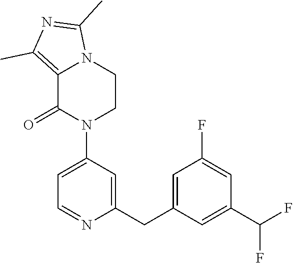 [SMILES: Cc1nc(C)n2c1C(=O)N(c1ccnc(Cc3cc(F)cc(C(F)F)c3)c1)CC2] |
| 19 | 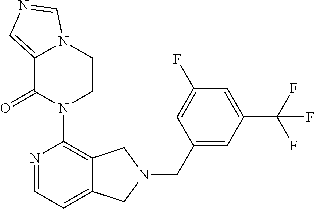 [SMILES: O=C1c2cncn2CCN1c1nccc2c1CN(Cc1cc(F)cc(C(F)(F)F)c1)C2] |
| 20 | 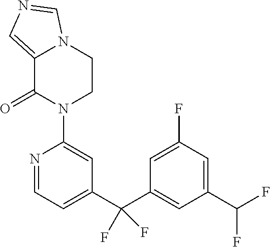 [SMILES: O=C1c2cncn2CCN1c1cc(C(F)(F)c2cc(F)cc(C(F)F)c2)ccn1] |
| 21 | 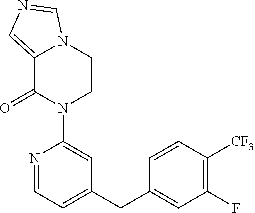 [SMILES: O=C1c2cncn2CCN1c1cc(Cc2ccc(C(F)(F)F)c(F)c2)ccn1] |
| 22 | 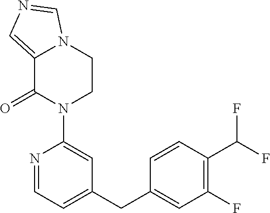 [SMILES: O=C1c2cncn2CCN1c1cc(Cc2ccc(C(F)F)c(F)c2)ccn1] |
| 23 | 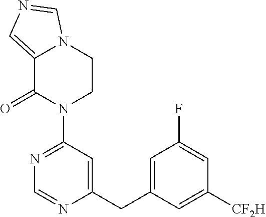 [SMILES: O=C1c2cncn2CCN1c1cc(Cc2cc(F)cc(C(F)F)c2)ncn1] |
| 24 | 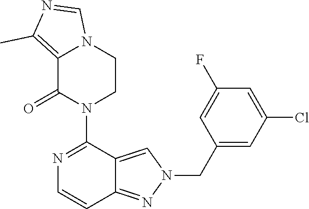 [SMILES: Cc1ncn2c1C(=O)N(c1nccc3nn(Cc4cc(F)cc(Cl)c4)cc13)CC2] |
| 25 | 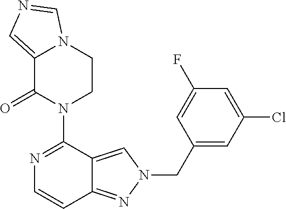 [SMILES: O=C1c2cncn2CCN1c1nccc2nn(Cc3cc(F)cc(Cl)c3)cc12] |
| 26 | 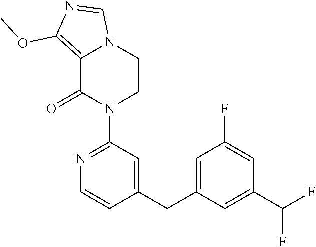 [SMILES: COc1ncn2c1C(=O)N(c1cc(Cc3cc(F)cc(C(F)F)c3)ccn1)CC2] |
| 27 | 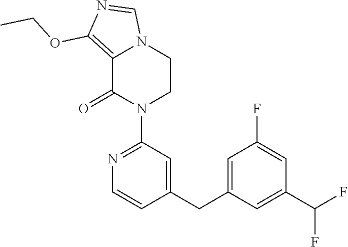 [SMILES: CCOc1ncn2c1C(=O)N(c1cc(Cc3cc(F)cc(C(F)F)c3)ccn1)CC2] |
| 28 | 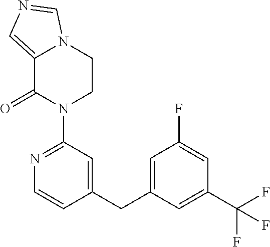 [SMILES: O=C1c2cncn2CCN1c1cc(Cc2cc(F)cc(C(F)(F)F)c2)ccn1] |
| 29 | 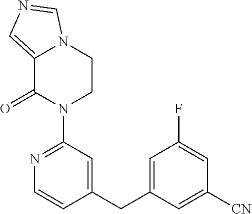 [SMILES: N#Cc1cc(F)cc(Cc2ccnc(N3CCn4cncc4C3=O)c2)c1] |
| 30 | 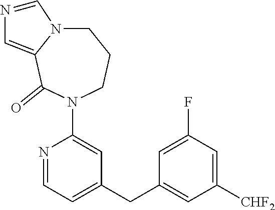 [SMILES: O=C1c2cncn2CCCN1c1cc(Cc2cc(F)cc(C(F)F)c2)ccn1] |
| 31 | 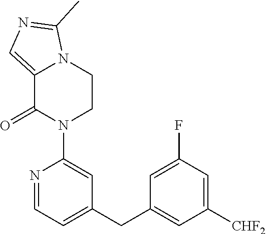 [SMILES: Cc1ncc2n1CCN(c1cc(Cc3cc(F)cc(C(F)F)c3)ccn1)C2=O] |
| 32 | 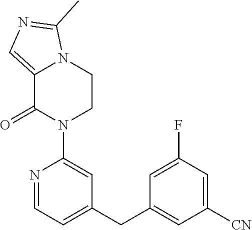 [SMILES: Cc1ncc2n1CCN(c1cc(Cc3cc(F)cc(C#N)c3)ccn1)C2=O] |
| 33 | 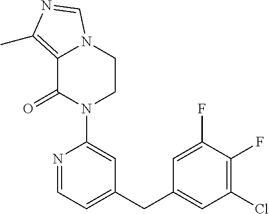 [SMILES: Cc1ncn2c1C(=O)N(c1cc(Cc3cc(F)c(F)c(Cl)c3)ccn1)CC2] |
| 34 | 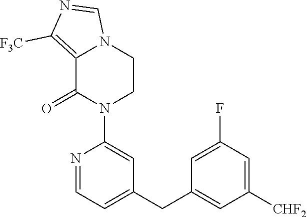 [SMILES: O=C1c2c(C(F)(F)F)ncn2CCN1c1cc(Cc2cc(F)cc(C(F)F)c2)ccn1] |
| 35 | 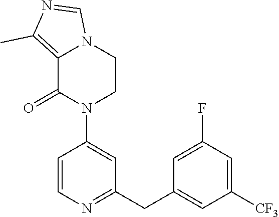 [SMILES: Cc1ncn2c1C(=O)N(c1ccnc(Cc3cc(F)cc(C(F)(F)F)c3)c1)CC2] |
| 36 | 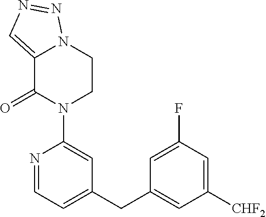 [SMILES: O=C1c2cnnn2CCN1c1cc(Cc2cc(F)cc(C(F)F)c2)ccn1] |
| 37 | 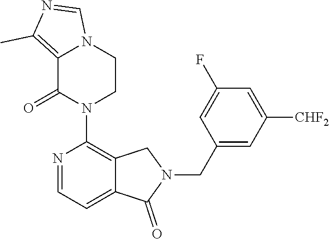 [SMILES: Cc1ncn2c1C(=O)N(c1nccc3c1CN(Cc1cc(F)cc(C(F)F)c1)C3=O)CC2] |
| 38 | 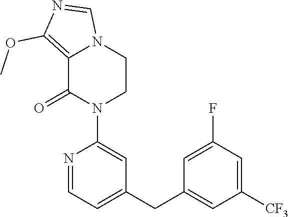 [SMILES: COc1ncn2c1C(=O)N(c1cc(Cc3cc(F)cc(C(F)(F)F)c3)ccn1)CC2] |
| 39 | 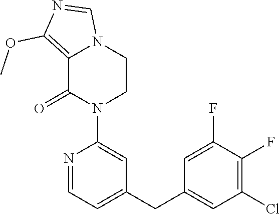 [SMILES: COc1ncn2c1C(=O)N(c1cc(Cc3cc(F)c(F)c(Cl)c3)ccn1)CC2] |
| 40 | 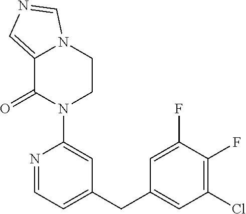 [SMILES: O=C1c2cncn2CCN1c1cc(Cc2cc(F)c(F)c(Cl)c2)ccn1] |
| 41 | 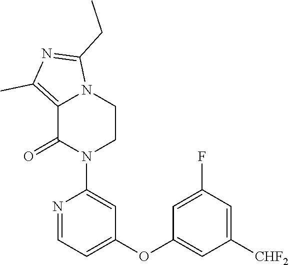 [SMILES: CCc1nc(C)c2n1CCN(c1cc(Oc3cc(F)cc(C(F)F)c3)ccn1)C2=O] |
| 42 | 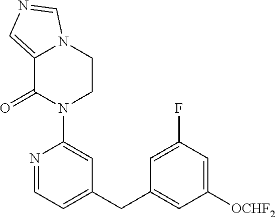 [SMILES: O=C1c2cncn2CCN1c1cc(Cc2cc(F)cc(OC(F)F)c2)ccn1] |
| 43 | 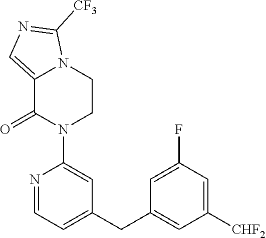 [SMILES: O=C1c2cnc(C(F)(F)F)n2CCN1c1cc(Cc2cc(F)cc(C(F)F)c2)ccn1] |
| 44 | 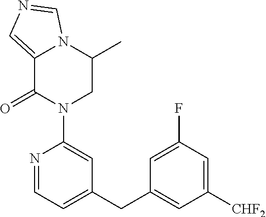 [SMILES: CC1CN(c2cc(Cc3cc(F)cc(C(F)F)c3)ccn2)C(=O)c2cncn21] |
| 45 | 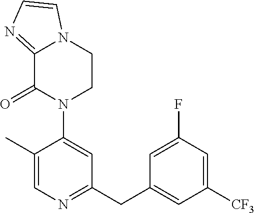 [SMILES: Cc1cnc(Cc2cc(F)cc(C(F)(F)F)c2)cc1N1CCn2ccnc2C1=O] |
| 46 | 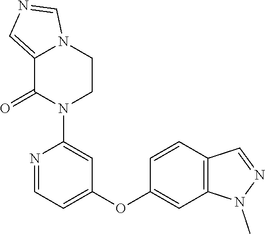 [SMILES: Cn1ncc2ccc(Oc3ccnc(N4CCn5cncc5C4=O)c3)cc21] |
| 47 | 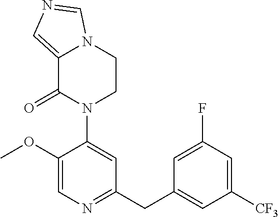 [SMILES: COc1cnc(Cc2cc(F)cc(C(F)(F)F)c2)cc1N1CCn2cncc2C1=O] |
| 48 | 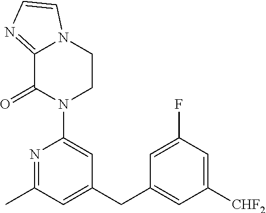 [SMILES: Cc1cc(Cc2cc(F)cc(C(F)F)c2)cc(N2CCn3ccnc3C2=O)n1] |
| 49 | 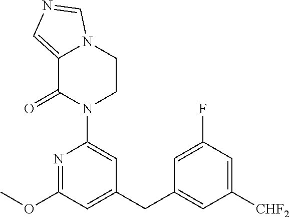 [SMILES: COc1cc(Cc2cc(F)cc(C(F)F)c2)cc(N2CCn3cncc3C2=O)n1] |
| 50 | 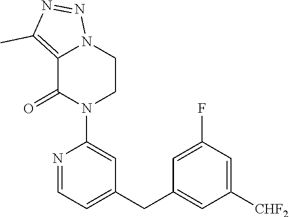 [SMILES: Cc1nnn2c1C(=O)N(c1cc(Cc3cc(F)cc(C(F)F)c3)ccn1)CC2] |
| 51 | 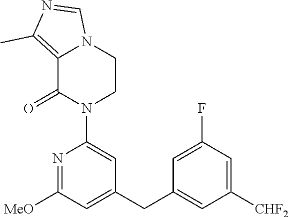 [SMILES: COc1cc(Cc2cc(F)cc(C(F)F)c2)cc(N2CCn3cnc(C)c3C2=O)n1] |
| 52 | 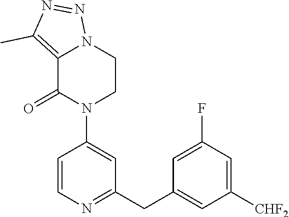 [SMILES: Cc1nnn2c1C(=O)N(c1ccnc(Cc3cc(F)cc(C(F)F)c3)c1)CC2] |
| 53 | 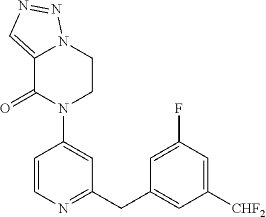 [SMILES: O=C1c2cnnn2CCN1c1ccnc(Cc2cc(F)cc(C(F)F)c2)c1] |
| 54 | 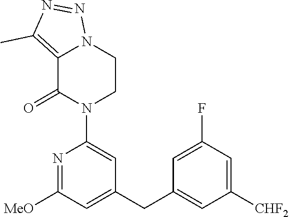 [SMILES: COc1cc(Cc2cc(F)cc(C(F)F)c2)cc(N2CCn3nnc(C)c3C2=O)n1] |
| 55 | 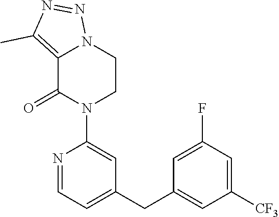 [SMILES: Cc1nnn2c1C(=O)N(c1cc(Cc3cc(F)cc(C(F)(F)F)c3)ccn1)CC2] |
| 56 | 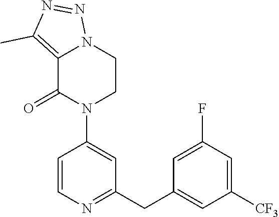 [SMILES: Cc1nnn2c1C(=O)N(c1ccnc(Cc3cc(F)cc(C(F)(F)F)c3)c1)CC2] |
| 57 | 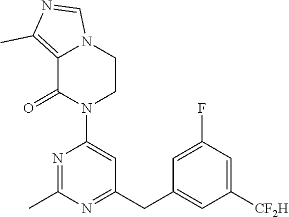 [SMILES: Cc1nc(Cc2cc(F)cc(C(F)F)c2)cc(N2CCn3cnc(C)c3C2=O)n1] |
| 58 | 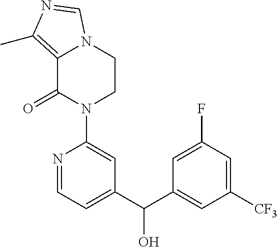 [SMILES: Cc1ncn2c1C(=O)N(c1cc(C(O)c3cc(F)cc(C(F)(F)F)c3)ccn1)CC2] |
| 59 | 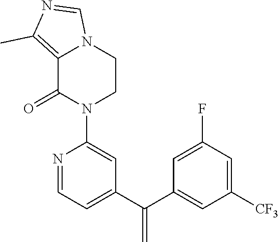 [SMILES: C=C(c1cc(F)cc(C(F)(F)F)c1)c1ccnc(N2CCn3cnc(C)c3C2=O)c1] |
| 60 | 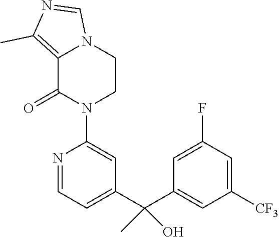 [SMILES: Cc1ncn2c1C(=O)N(c1cc(C(C)(O)c3cc(F)cc(C(F)(F)F)c3)ccn1)CC2] |
| 61 | 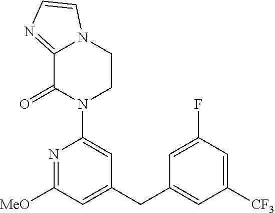 [SMILES: COc1cc(Cc2cc(F)cc(C(F)(F)F)c2)cc(N2CCn3ccnc3C2=O)n1] |
| 62 | 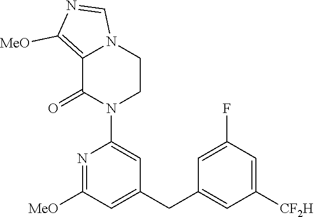 [SMILES: COc1cc(Cc2cc(F)cc(C(F)F)c2)cc(N2CCn3cnc(OC)c3C2=O)n1] |
| 63 | 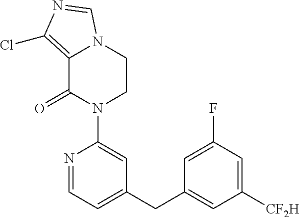 [SMILES: O=C1c2c(Cl)ncn2CCN1c1cc(Cc2cc(F)cc(C(F)F)c2)ccn1] |
| 64 | 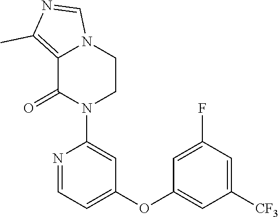 [SMILES: Cc1ncn2c1C(=O)N(c1cc(Oc3cc(F)cc(C(F)(F)F)c3)ccn1)CC2] |
| 65 | 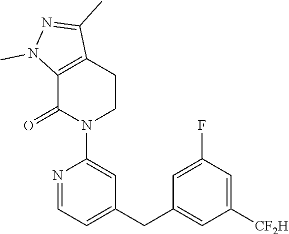 [SMILES: Cc1nn(C)c2c1CCN(c1cc(Cc3cc(F)cc(C(F)F)c3)ccn1)C2=O] |
| 66 | 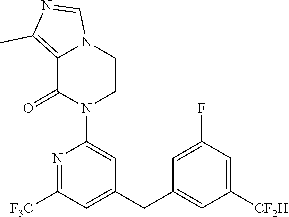 [SMILES: Cc1ncn2c1C(=O)N(c1cc(Cc3cc(F)cc(C(F)F)c3)cc(C(F)(F)F)n1)CC2] |
| 67 | 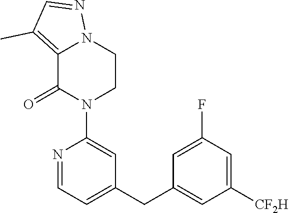 [SMILES: Cc1cnn2c1C(=O)N(c1cc(Cc3cc(F)cc(C(F)F)c3)ccn1)CC2] |
| 68 | 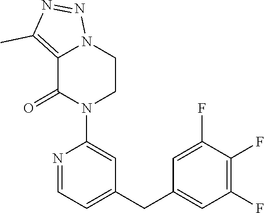 [SMILES: Cc1nnn2c1C(=O)N(c1cc(Cc3cc(F)c(F)c(F)c3)ccn1)CC2] |
| 69 | 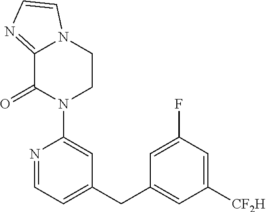 [SMILES: O=C1c2nccn2CCN1c1cc(Cc2cc(F)cc(C(F)F)c2)ccn1] |
| 70 | 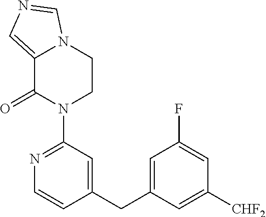 [SMILES: O=C1c2cncn2CCN1c1cc(Cc2cc(F)cc(C(F)F)c2)ccn1] |
| 71 | 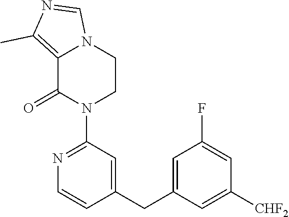 [SMILES: Cc1ncn2c1C(=O)N(c1cc(Cc3cc(F)cc(C(F)F)c3)ccn1)CC2] |
| 72 | 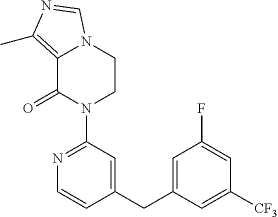 [SMILES: Cc1ncn2c1C(=O)N(c1cc(Cc3cc(F)cc(C(F)(F)F)c3)ccn1)CC2] |
| 73a/73b | 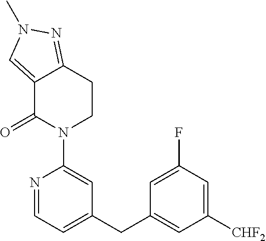 [SMILES: Cn1cc2c(n1)CCN(c1cc(Cc3cc(F)cc(C(F)F)c3)ccn1)C2=O] 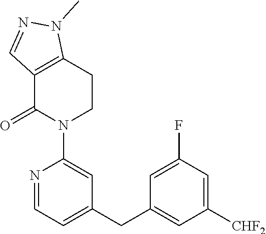 [SMILES: Cn1ncc2c1CCN(c1cc(Cc3cc(F)cc(C(F)F)c3)ccn1)C2=O] |
| 74 | 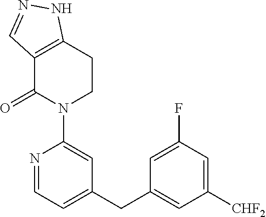 [SMILES: O=C1c2cn[nH]c2CCN1c1cc(Cc2cc(F)cc(C(F)F)c2)ccn1] |
| 75 | 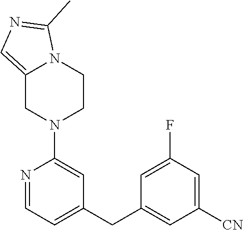 [SMILES: Cc1ncc2n1CCN(c1cc(Cc3cc(F)cc(C#N)c3)ccn1)C2] |
| 76 | 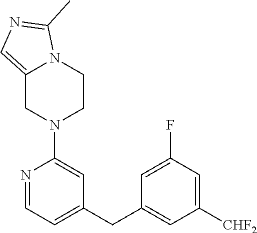 [SMILES: Cc1ncc2n1CCN(c1cc(Cc3cc(F)cc(C(F)F)c3)ccn1)C2] |
| 77 | 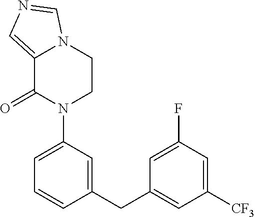 [SMILES: O=C1c2cncn2CCN1c1cccc(Cc2cc(F)cc(C(F)(F)F)c2)c1] |
| 78 | 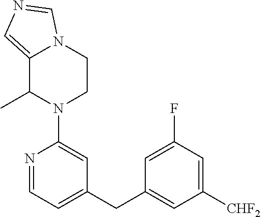 [SMILES: CC1c2cncn2CCN1c1cc(Cc2cc(F)cc(C(F)F)c2)ccn1] |
| 79 | 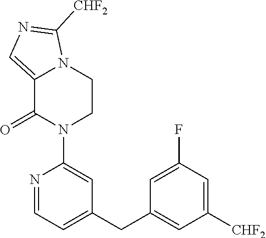 [SMILES: O=C1c2cnc(C(F)F)n2CCN1c1cc(Cc2cc(F)cc(C(F)F)c2)ccn1] |
| 80 | 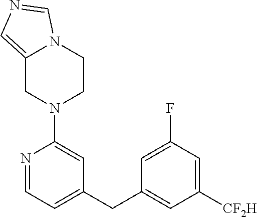 [SMILES: Fc1cc(Cc2ccnc(N3CCn4cncc4C3)c2)cc(C(F)F)c1] |
| 81 | 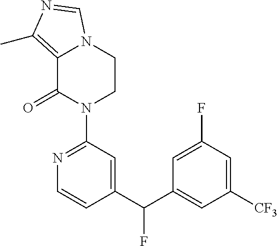 [SMILES: Cc1ncn2c1C(=O)N(c1cc(C(F)c3cc(F)cc(C(F)(F)F)c3)ccn1)CC2] |
| 82 | 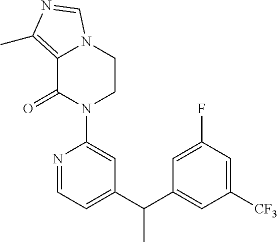 [SMILES: Cc1ncn2c1C(=O)N(c1cc(C(C)c3cc(F)cc(C(F)(F)F)c3)ccn1)CC2] |
| 83 | 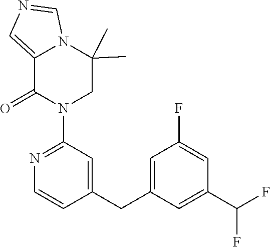 [SMILES: CC1(C)CN(c2cc(Cc3cc(F)cc(C(F)F)c3)ccn2)C(=O)c2cncn21] |
| 84 | 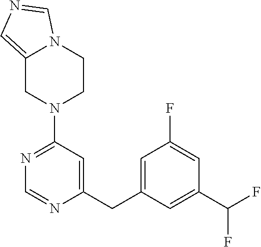 [SMILES: Fc1cc(Cc2cc(N3CCn4cncc4C3)ncn2)cc(C(F)F)c1] |
| 85 | 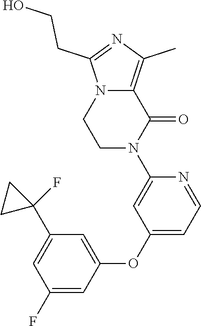 [SMILES: Cc1nc(CCO)n2c1C(=O)N(c1cc(Oc3cc(F)cc(C4(F)CC4)c3)ccn1)CC2] |
| 86 | 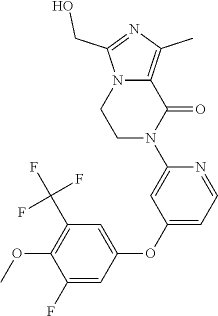 [SMILES: COc1c(F)cc(Oc2ccnc(N3CCn4c(CO)nc(C)c4C3=O)c2)cc1C(F)(F)F] |
| 87 | 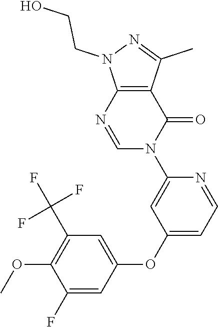 [SMILES: COc1c(F)cc(Oc2ccnc(-n3cnc4c(c(C)nn4CCO)c3=O)c2)cc1C(F)(F)F] |
| 88 | 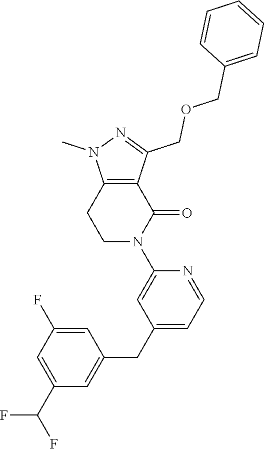 [SMILES: Cn1nc(COCc2ccccc2)c2c1CCN(c1cc(Cc3cc(F)cc(C(F)F)c3)ccn1)C2=O] |
| 89 | 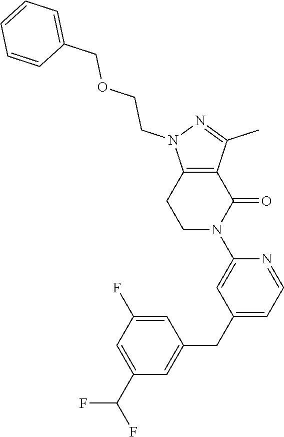 [SMILES: Cc1nn(CCOCc2ccccc2)c2c1C(=O)N(c1cc(Cc3cc(F)cc(C(F)F)c3)ccn1)CC2] |
| 90 | 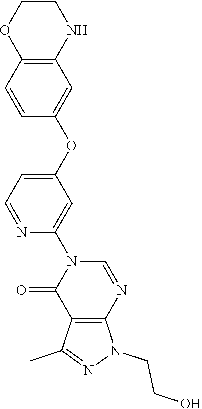 [SMILES: Cc1nn(CCO)c2ncn(-c3cc(Oc4ccc5c(c4)NCCO5)ccn3)c(=O)c12] |
| 91 | 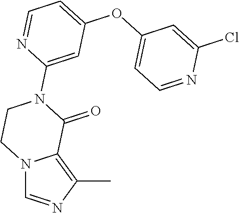 [SMILES: Cc1ncn2c1C(=O)N(c1cc(Oc3ccnc(Cl)c3)ccn1)CC2] |
| 92 | 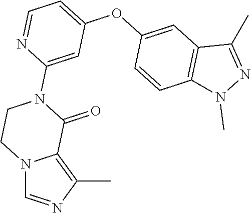 [SMILES: Cc1ncn2c1C(=O)N(c1cc(Oc3ccc4c(c3)c(C)nn4C)ccn1)CC2] |
| 93 | 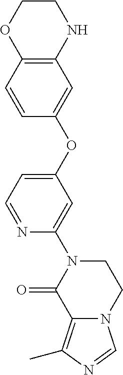 [SMILES: Cc1ncn2c1C(=O)N(c1cc(Oc3ccc4c(c3)NCCO4)ccn1)CC2] |
| 94 | 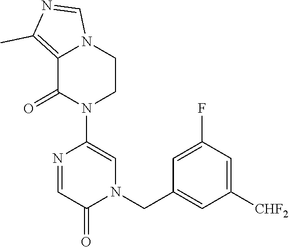 [SMILES: Cc1ncn2c1C(=O)N(c1cn(Cc3cc(F)cc(C(F)F)c3)c(=O)cn1)CC2] |
| 95 | 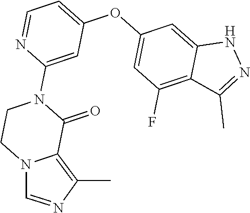 [SMILES: Cc1ncn2c1C(=O)N(c1cc(Oc3cc(F)c4c(C)n[nH]c4c3)ccn1)CC2] |
| 96 | 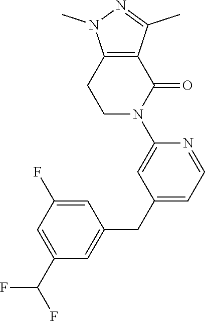 [SMILES: Cc1nn(C)c2c1C(=O)N(c1cc(Cc3cc(F)cc(C(F)F)c3)ccn1)CC2] |
| 97 | 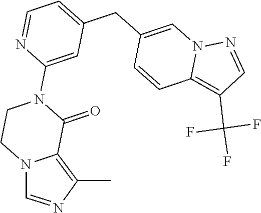 [SMILES: Cc1ncn2c1C(=O)N(c1cc(Cc3ccc4c(C(F)(F)F)cnn4c3)ccn1)CC2] |
| 98 | 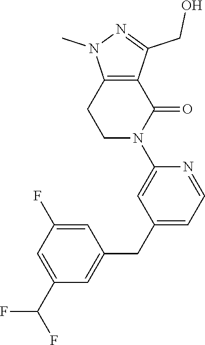 [SMILES: Cn1nc(CO)c2c1CCN(c1cc(Cc3cc(F)cc(C(F)F)c3)ccn1)C2=O] |
| 99 | 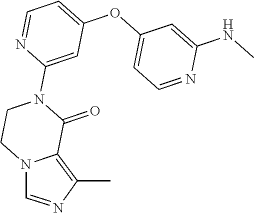 [SMILES: CNc1cc(Oc2ccnc(N3CCn4cnc(C)c4C3=O)c2)ccn1] |
| 100 | 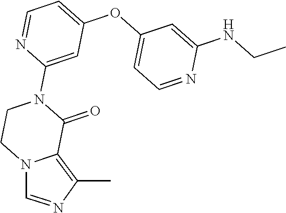 [SMILES: CCNc1cc(Oc2ccnc(N3CCn4cnc(C)c4C3=O)c2)ccn1] |
| 101 | 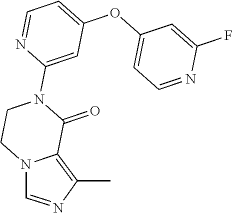 [SMILES: Cc1ncn2c1C(=O)N(c1cc(Oc3ccnc(F)c3)ccn1)CC2] |
| 102 | 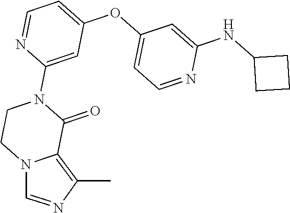 [SMILES: Cc1ncn2c1C(=O)N(c1cc(Oc3ccnc(NC4CCC4)c3)ccn1)CC2] |
| 103 | 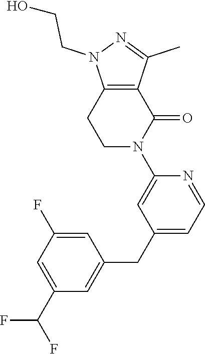 [SMILES: Cc1nn(CCO)c2c1C(=O)N(c1cc(Cc3cc(F)cc(C(F)F)c3)ccn1)CC2] |
| 104 | 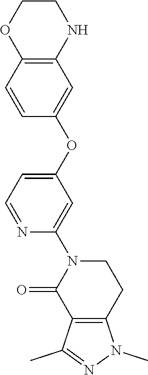 [SMILES: Cc1nn(C)c2c1C(=O)N(c1cc(Oc3ccc4c(c3)NCCO4)ccn1)CC2] |
| 105 | 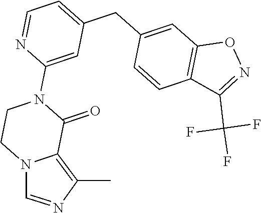 [SMILES: Cc1ncn2c1C(=O)N(c1cc(Cc3ccc4c(C(F)(F)F)noc4c3)ccn1)CC2] |
| 106 | 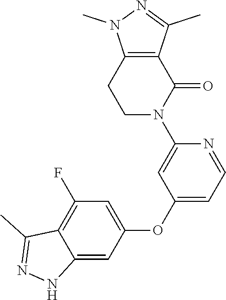 [SMILES: Cc1nn(C)c2c1C(=O)N(c1cc(Oc3cc(F)c4c(C)n[nH]c4c3)ccn1)CC2] |
| 107 | 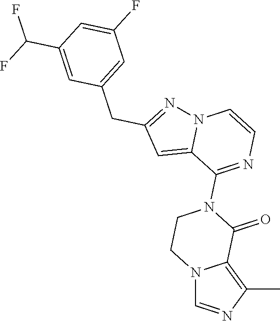 [SMILES: Cc1ncn2c1C(=O)N(c1nccn3nc(Cc4cc(F)cc(C(F)F)c4)cc13)CC2] |
| 108 | 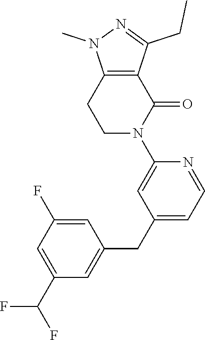 [SMILES: CCc1nn(C)c2c1C(=O)N(c1cc(Cc3cc(F)cc(C(F)F)c3)ccn1)CC2] |
| 109 | 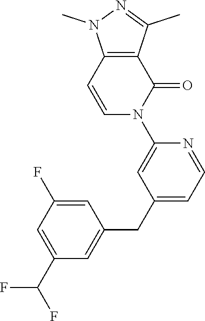 [SMILES: Cc1nn(C)c2ccn(-c3cc(Cc4cc(F)cc(C(F)F)c4)ccn3)c(=O)c12] |
| 110 | 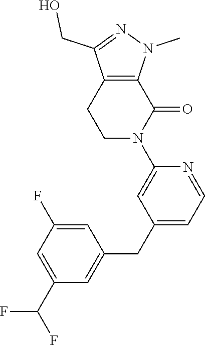 [SMILES: Cn1nc(CO)c2c1C(=O)N(c1cc(Cc3cc(F)cc(C(F)F)c3)ccn1)CC2] |
| 111 | 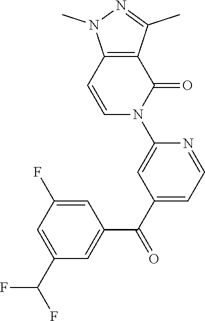 [SMILES: Cc1nn(C)c2ccn(-c3cc(C(=O)c4cc(F)cc(C(F)F)c4)ccn3)c(=O)c12] |
| 112 | 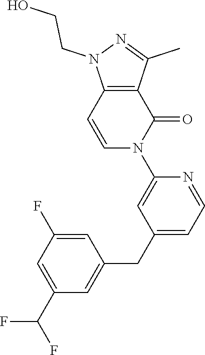 [SMILES: Cc1nn(CCO)c2ccn(-c3cc(Cc4cc(F)cc(C(F)F)c4)ccn3)c(=O)c12] |
| 113 | 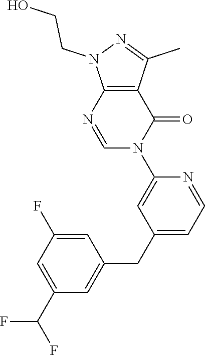 [SMILES: Cc1nn(CCO)c2ncn(-c3cc(Cc4cc(F)cc(C(F)F)c4)ccn3)c(=O)c12] |
| 114 | 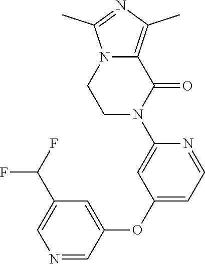 [SMILES: Cc1nc(C)n2c1C(=O)N(c1cc(Oc3cncc(C(F)F)c3)ccn1)CC2] |
| 115 | 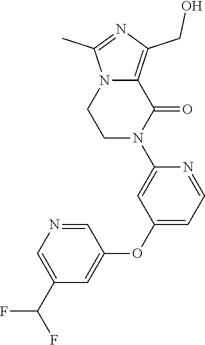 [SMILES: Cc1nc(CO)c2n1CCN(c1cc(Oc3cncc(C(F)F)c3)ccn1)C2=O] |
| 116 | 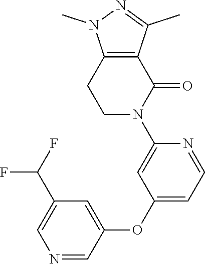 [SMILES: Cc1nn(C)c2c1C(=O)N(c1cc(Oc3cncc(C(F)F)c3)ccn1)CC2] |
| 117 | 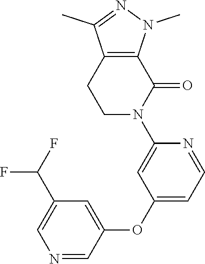 [SMILES: Cc1nn(C)c2c1CCN(c1cc(Oc3cncc(C(F)F)c3)ccn1)C2=O] |
| 118 | 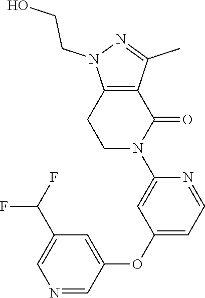 [SMILES: Cc1nn(CCO)c2c1C(=O)N(c1cc(Oc3cncc(C(F)F)c3)ccn1)CC2] |
| 119 | 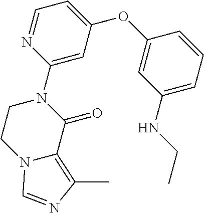 [SMILES: CCNc1cccc(Oc2ccnc(N3CCn4cnc(C)c4C3=O)c2)c1] |
| 120 | 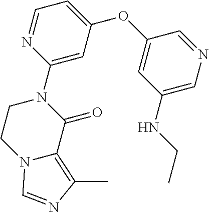 [SMILES: CCNc1cncc(Oc2ccnc(N3CCn4cnc(C)c4C3=O)c2)c1] |
| 121 | 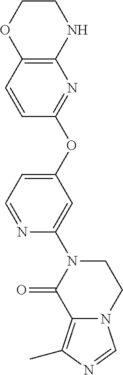 [SMILES: Cc1ncn2c1C(=O)N(c1cc(Oc3ccc4c(n3)NCCO4)ccn1)CC2] |
| 122 | 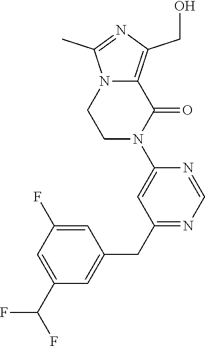 [SMILES: Cc1nc(CO)c2n1CCN(c1cc(Cc3cc(F)cc(C(F)F)c3)ncn1)C2=O] |
| 123 | 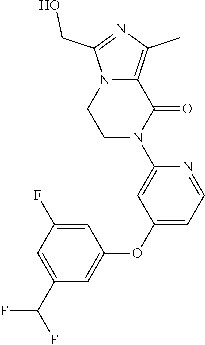 [SMILES: Cc1nc(CO)n2c1C(=O)N(c1cc(Oc3cc(F)cc(C(F)F)c3)ccn1)CC2] |
| 124 | 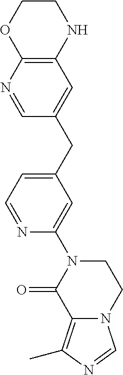 [SMILES: Cc1ncn2c1C(=O)N(c1cc(Cc3cnc4c(c3)NCCO4)ccn1)CC2] |
| 125 | 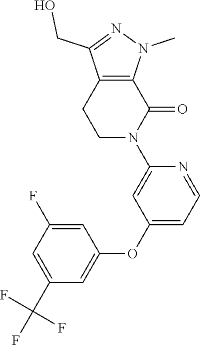 [SMILES: Cn1nc(CO)c2c1C(=O)N(c1cc(Oc3cc(F)cc(C(F)(F)F)c3)ccn1)CC2] |
| 126 | 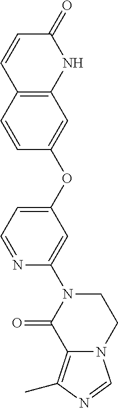 [SMILES: Cc1ncn2c1C(=O)N(c1cc(Oc3ccc4ccc(=O)[nH]c4c3)ccn1)CC2] |
| 127 | 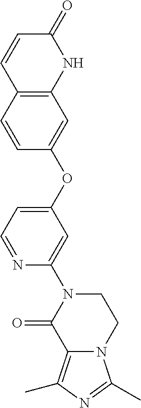 [SMILES: Cc1nc(C)n2c1C(=O)N(c1cc(Oc3ccc4ccc(=O)[nH]c4c3)ccn1)CC2] |
| 128 | 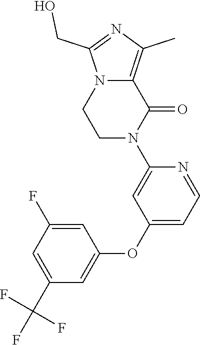 [SMILES: Cc1nc(CO)n2c1C(=O)N(c1cc(Oc3cc(F)cc(C(F)(F)F)c3)ccn1)CC2] |
| 129 | 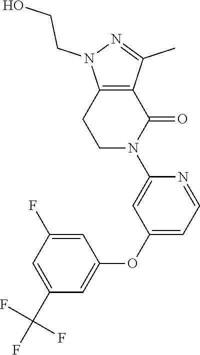 [SMILES: Cc1nn(CCO)c2c1C(=O)N(c1cc(Oc3cc(F)cc(C(F)(F)F)c3)ccn1)CC2] |
| 130 | 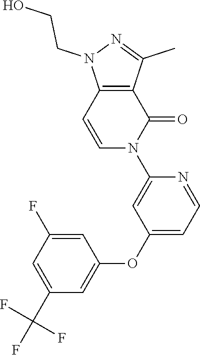 [SMILES: Cc1nn(CCO)c2ccn(-c3cc(Oc4cc(F)cc(C(F)(F)F)c4)ccn3)c(=O)c12] |
| 131 | 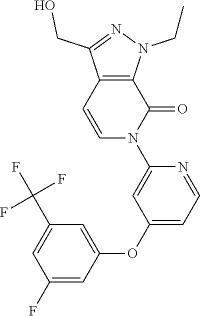 [SMILES: CCn1nc(CO)c2ccn(-c3cc(Oc4cc(F)cc(C(F)(F)F)c4)ccn3)c(=O)c21] |
| 132 | 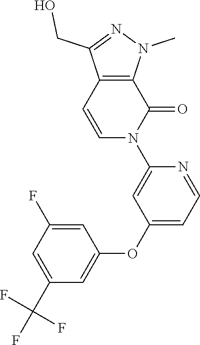 [SMILES: Cn1nc(CO)c2ccn(-c3cc(Oc4cc(F)cc(C(F)(F)F)c4)ccn3)c(=O)c21] |
| 133 | 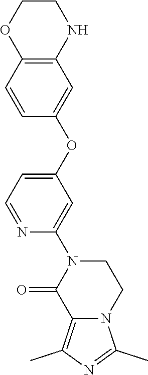 [SMILES: Cc1nc(C)n2c1C(=O)N(c1cc(Oc3ccc4c(c3)NCCO4)ccn1)CC2] |
| 134 | 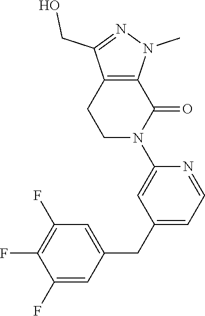 [SMILES: Cn1nc(CO)c2c1C(=O)N(c1cc(Cc3cc(F)c(F)c(F)c3)ccn1)CC2] |
| 135 | 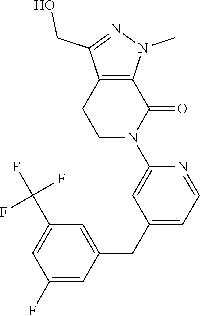 [SMILES: Cn1nc(CO)c2c1C(=O)N(c1cc(Cc3cc(F)cc(C(F)(F)F)c3)ccn1)CC2] |
| 136 | 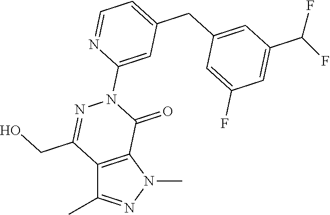 [SMILES: Cc1nn(C)c2c(=O)n(-c3cc(Cc4cc(F)cc(C(F)F)c4)ccn3)nc(CO)c12] |
| 137 | 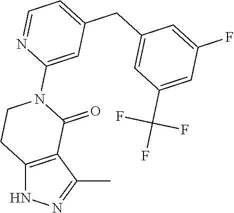 [SMILES: Cc1n[nH]c2c1C(=O)N(c1cc(Cc3cc(F)cc(C(F)(F)F)c3)ccn1)CC2] |
| 138 | 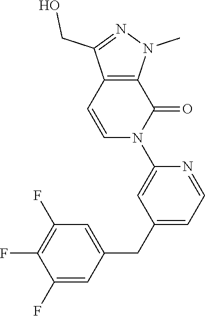 [SMILES: Cn1nc(CO)c2ccn(-c3cc(Cc4cc(F)c(F)c(F)c4)ccn3)c(=O)c21] |
| 139 | 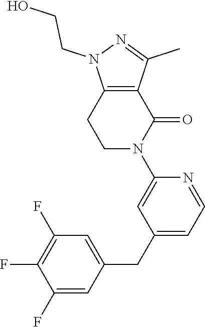 [SMILES: Cc1nn(CCO)c2c1C(=O)N(c1cc(Cc3cc(F)c(F)c(F)c3)ccn1)CC2] |
| 140 | 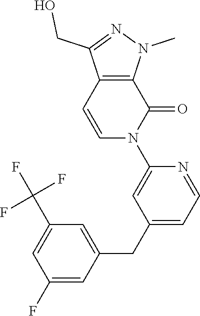 [SMILES: Cn1nc(CO)c2ccn(-c3cc(Cc4cc(F)cc(C(F)(F)F)c4)ccn3)c(=O)c21] |
| 141 | 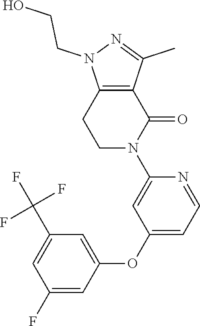 [SMILES: Cc1nn(CCO)c2c1C(=O)N(c1cc(Oc3cc(F)cc(C(F)(F)F)c3)ccn1)CC2] |
| 142 | 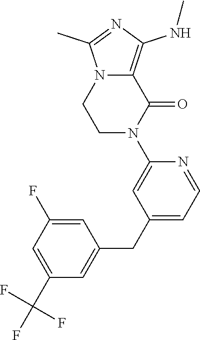 [SMILES: CNc1nc(C)n2c1C(=O)N(c1cc(Cc3cc(F)cc(C(F)(F)F)c3)ccn1)CC2] |
| 143 | 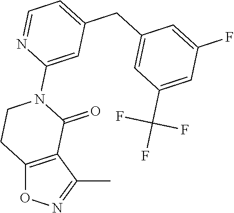 [SMILES: Cc1noc2c1C(=O)N(c1cc(Cc3cc(F)cc(C(F)(F)F)c3)ccn1)CC2] |
| 144 | 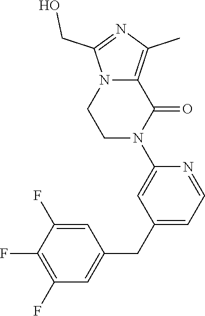 [SMILES: Cc1nc(CO)n2c1C(=O)N(c1cc(Cc3cc(F)c(F)c(F)c3)ccn1)CC2] |
| 145 | 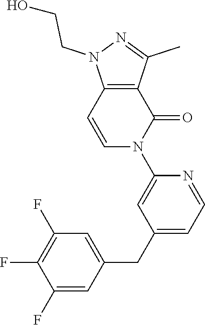 [SMILES: Cc1nn(CCO)c2ccn(-c3cc(Cc4cc(F)c(F)c(F)c4)ccn3)c(=O)c12] |
| 146 | 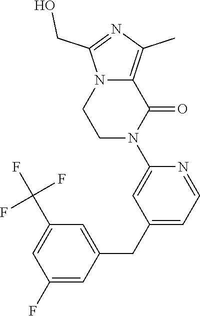 [SMILES: Cc1nc(CO)n2c1C(=O)N(c1cc(Cc3cc(F)cc(C(F)(F)F)c3)ccn1)CC2] |
| 147 | 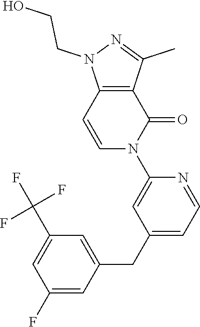 [SMILES: Cc1nn(CCO)c2ccn(-c3cc(Cc4cc(F)cc(C(F)(F)F)c4)ccn3)c(=O)c12] |
| 148 | 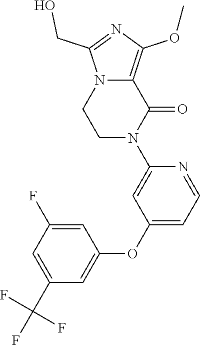 [SMILES: COc1nc(CO)n2c1C(=O)N(c1cc(Oc3cc(F)cc(C(F)(F)F)c3)ccn1)CC2] |
| 149 | 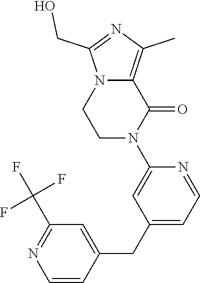 [SMILES: Cc1nc(CO)n2c1C(=O)N(c1cc(Cc3ccnc(C(F)(F)F)c3)ccn1)CC2] |
| 150 | 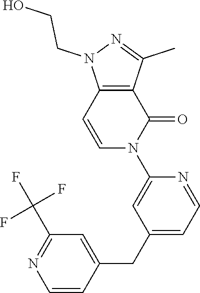 [SMILES: Cc1nn(CCO)c2ccn(-c3cc(Cc4ccnc(C(F)(F)F)c4)ccn3)c(=O)c12] |
| 151 | 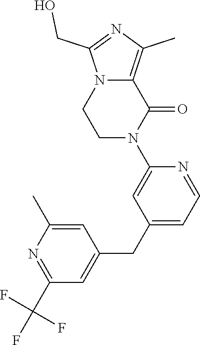 [SMILES: Cc1cc(Cc2ccnc(N3CCn4c(CO)nc(C)c4C3=O)c2)cc(C(F)(F)F)n1] |
| 152 | 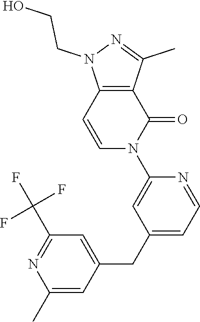 [SMILES: Cc1cc(Cc2ccnc(-n3ccc4c(c(C)nn4CCO)c3=O)c2)cc(C(F)(F)F)n1] |
| 153 | 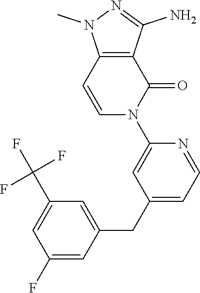 [SMILES: Cn1nc(N)c2c(=O)n(-c3cc(Cc4cc(F)cc(C(F)(F)F)c4)ccn3)ccc21] |
| 154 | 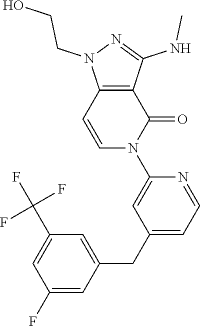 [SMILES: CNc1nn(CCO)c2ccn(-c3cc(Cc4cc(F)cc(C(F)(F)F)c4)ccn3)c(=O)c12] |
| 155 | 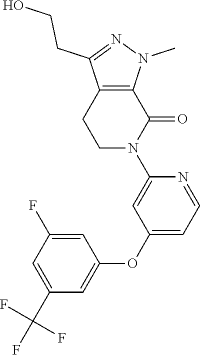 [SMILES: Cn1nc(CCO)c2c1C(=O)N(c1cc(Oc3cc(F)cc(C(F)(F)F)c3)ccn1)CC2] |
| 156 | 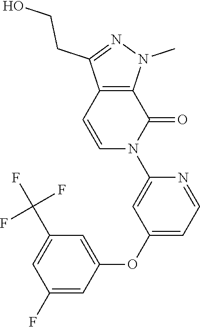 [SMILES: Cn1nc(CCO)c2ccn(-c3cc(Oc4cc(F)cc(C(F)(F)F)c4)ccn3)c(=O)c21] |
| 157 | 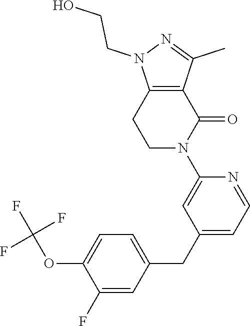 [SMILES: Cc1nn(CCO)c2c1C(=O)N(c1cc(Cc3ccc(OC(F)(F)F)c(F)c3)ccn1)CC2] |
| 158 | 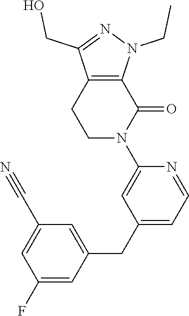 [SMILES: CCn1nc(CO)c2c1C(=O)N(c1cc(Cc3cc(F)cc(C#N)c3)ccn1)CC2] |
| 159 | 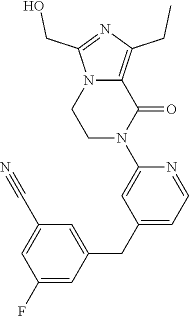 [SMILES: CCc1nc(CO)n2c1C(=O)N(c1cc(Cc3cc(F)cc(C#N)c3)ccn1)CC2] |
| 160 | 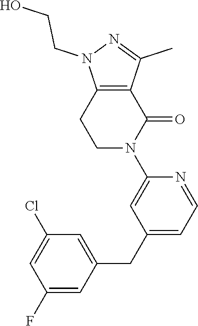 [SMILES: Cc1nn(CCO)c2c1C(=O)N(c1cc(Cc3cc(F)cc(Cl)c3)ccn1)CC2] |
| 161 | 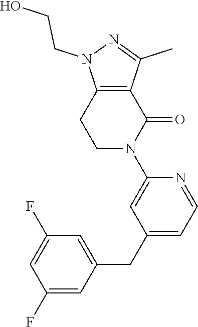 [SMILES: Cc1nn(CCO)c2c1C(=O)N(c1cc(Cc3cc(F)cc(F)c3)ccn1)CC2] |
| 162 | 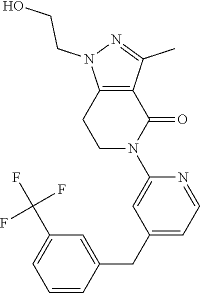 [SMILES: Cc1nn(CCO)c2c1C(=O)N(c1cc(Cc3cccc(C(F)(F)F)c3)ccn1)CC2] |
| 163 | 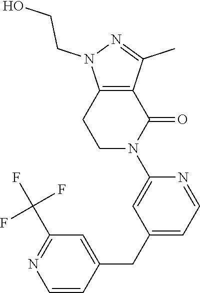 [SMILES: Cc1nn(CCO)c2c1C(=O)N(c1cc(Cc3ccnc(C(F)(F)F)c3)ccn1)CC2] |
| 164 | 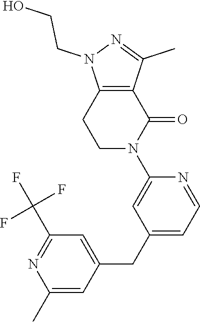 [SMILES: Cc1cc(Cc2ccnc(N3CCc4c(c(C)nn4CCO)C3=O)c2)cc(C(F)(F)F)n1] |
| 165 | 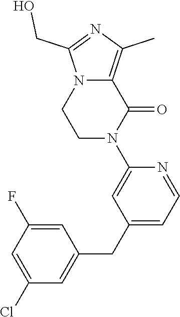 [SMILES: Cc1nc(CO)n2c1C(=O)N(c1cc(Cc3cc(F)cc(Cl)c3)ccn1)CC2] |
| 166 | 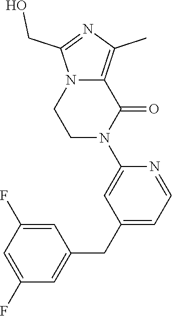 [SMILES: Cc1nc(CO)n2c1C(=O)N(c1cc(Cc3cc(F)cc(F)c3)ccn1)CC2] |
| 167 | 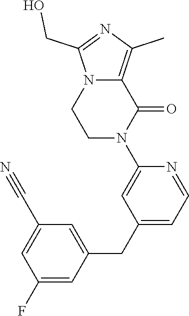 [SMILES: Cc1nc(CO)n2c1C(=O)N(c1cc(Cc3cc(F)cc(C#N)c3)ccn1)CC2] |
| 168 | 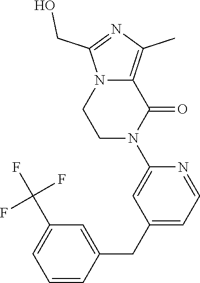 [SMILES: Cc1nc(CO)n2c1C(=O)N(c1cc(Cc3cccc(C(F)(F)F)c3)ccn1)CC2] |
| 169 | 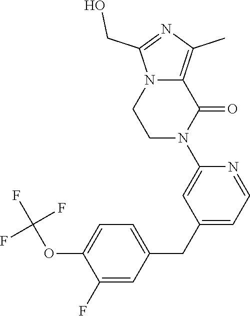 [SMILES: Cc1nc(CO)n2c1C(=O)N(c1cc(Cc3ccc(OC(F)(F)F)c(F)c3)ccn1)CC2] |
| 170 | 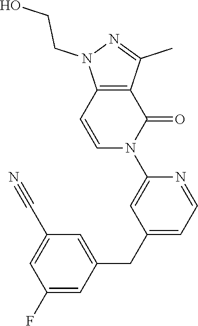 [SMILES: Cc1nn(CCO)c2ccn(-c3cc(Cc4cc(F)cc(C#N)c4)ccn3)c(=O)c12] |
| 171 | 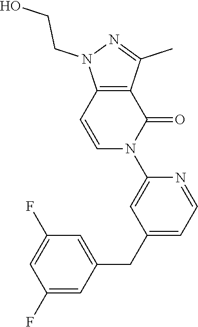 [SMILES: Cc1nn(CCO)c2ccn(-c3cc(Cc4cc(F)cc(F)c4)ccn3)c(=O)c12] |
| 172 | 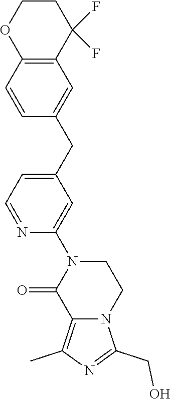 [SMILES: Cc1nc(CO)n2c1C(=O)N(c1cc(Cc3ccc4c(c3)C(F)(F)CCO4)ccn1)CC2] |
| 173 | 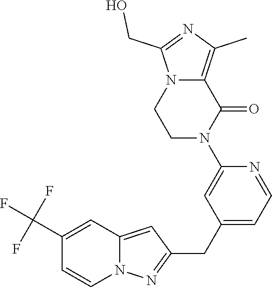 [SMILES: Cc1nc(CO)n2c1C(=O)N(c1cc(Cc3cc4cc(C(F)(F)F)ccn4n3)ccn1)CC2] |
| 174 | 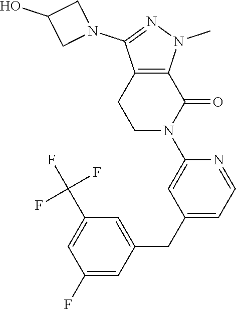 [SMILES: Cn1nc(N2CC(O)C2)c2c1C(=O)N(c1cc(Cc3cc(F)cc(C(F)(F)F)c3)ccn1)CC2] |
| 175 | 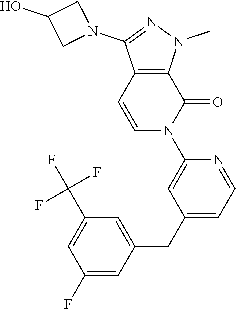 [SMILES: Cn1nc(N2CC(O)C2)c2ccn(-c3cc(Cc4cc(F)cc(C(F)(F)F)c4)ccn3)c(=O)c21] |
| 176 | 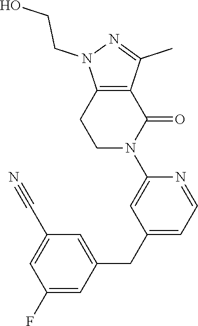 [SMILES: Cc1nn(CCO)c2c1C(=O)N(c1cc(Cc3cc(F)cc(C#N)c3)ccn1)CC2] |
| 177 | 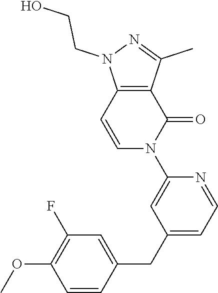 [SMILES: COc1ccc(Cc2ccnc(-n3ccc4c(c(C)nn4CCO)c3=O)c2)cc1F] |
| 178 | 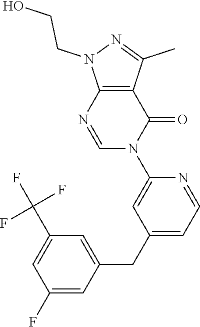 [SMILES: Cc1nn(CCO)c2ncn(-c3cc(Cc4cc(F)cc(C(F)(F)F)c4)ccn3)c(=O)c12] |
| 179 | 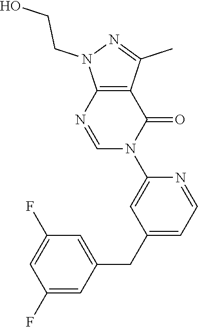 [SMILES: Cc1nn(CCO)c2ncn(-c3cc(Cc4cc(F)cc(F)c4)ccn3)c(=O)c12] |
| 180 | 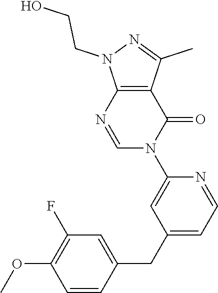 [SMILES: COc1ccc(Cc2ccnc(-n3cnc4c(c(C)nn4CCO)c3=O)c2)cc1F] |
| 181 | 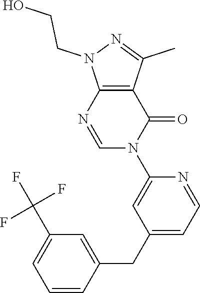 [SMILES: Cc1nn(CCO)c2ncn(-c3cc(Cc4cccc(C(F)(F)F)c4)ccn3)c(=O)c12] |
| 182 | 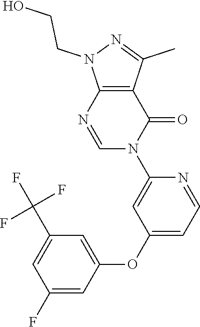 [SMILES: Cc1nn(CCO)c2ncn(-c3cc(Oc4cc(F)cc(C(F)(F)F)c4)ccn3)c(=O)c12] |
| 183 | 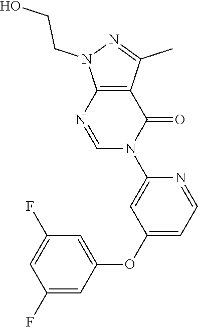 [SMILES: Cc1nn(CCO)c2ncn(-c3cc(Oc4cc(F)cc(F)c4)ccn3)c(=O)c12] |
| 184 | 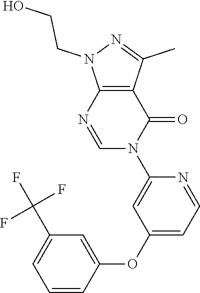 [SMILES: Cc1nn(CCO)c2ncn(-c3cc(Oc4cccc(C(F)(F)F)c4)ccn3)c(=O)c12] |
| 185 | 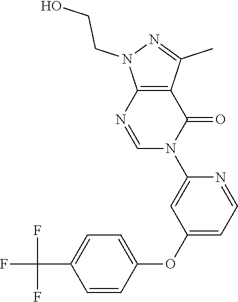 [SMILES: Cc1nn(CCO)c2ncn(-c3cc(Oc4ccc(C(F)(F)F)cc4)ccn3)c(=O)c12] |
| 186 | 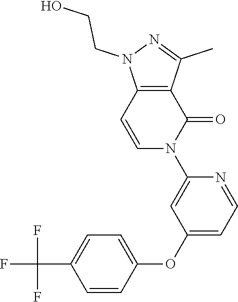 [SMILES: Cc1nn(CCO)c2ccn(-c3cc(Oc4ccc(C(F)(F)F)cc4)ccn3)c(=O)c12] |
| 187 | 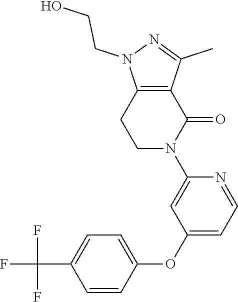 [SMILES: Cc1nn(CCO)c2c1C(=O)N(c1cc(Oc3ccc(C(F)(F)F)cc3)ccn1)CC2] |
| 188 | 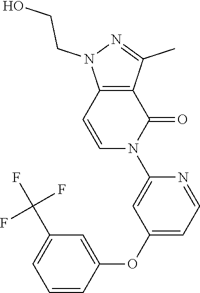 [SMILES: Cc1nn(CCO)c2ccn(-c3cc(Oc4cccc(C(F)(F)F)c4)ccn3)c(=O)c12] |
| 189 | 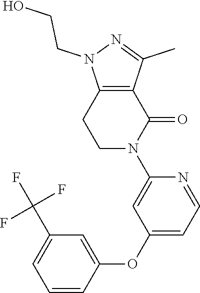 [SMILES: Cc1nn(CCO)c2c1C(=O)N(c1cc(Oc3cccc(C(F)(F)F)c3)ccn1)CC2] |
| 190 | 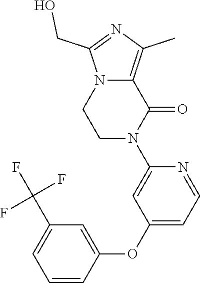 [SMILES: Cc1nc(CO)n2c1C(=O)N(c1cc(Oc3cccc(C(F)(F)F)c3)ccn1)CC2] |
| 191 | 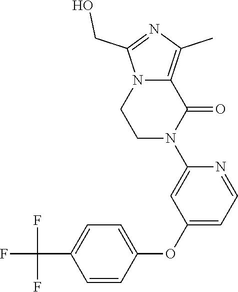 [SMILES: Cc1nc(CO)n2c1C(=O)N(c1cc(Oc3ccc(C(F)(F)F)cc3)ccn1)CC2] |
| 192 | 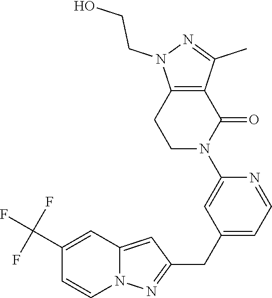 [SMILES: Cc1nn(CCO)c2c1C(=O)N(c1cc(Cc3cc4cc(C(F)(F)F)ccn4n3)ccn1)CC2] |
| 193 | 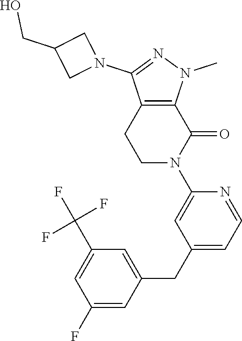 [SMILES: Cn1nc(N2CC(CO)C2)c2c1C(=O)N(c1cc(Cc3cc(F)cc(C(F)(F)F)c3)ccn1)CC2] |
| 194 | 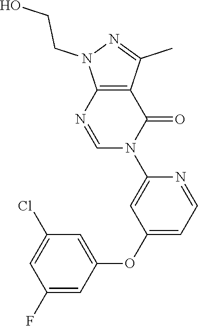 [SMILES: Cc1nn(CCO)c2ncn(-c3cc(Oc4cc(F)cc(Cl)c4)ccn3)c(=O)c12] |
| 195 | 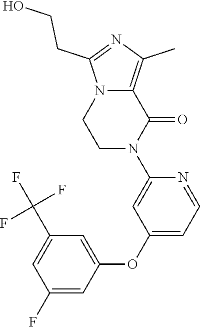 [SMILES: Cc1nc(CCO)n2c1C(=O)N(c1cc(Oc3cc(F)cc(C(F)(F)F)c3)ccn1)CC2] |
| 196 | 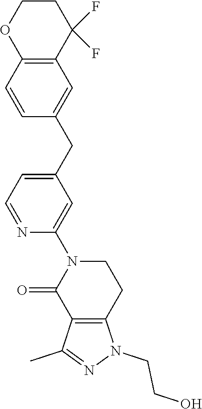 [SMILES: Cc1nn(CCO)c2c1C(=O)N(c1cc(Cc3ccc4c(c3)C(F)(F)CCO4)ccn1)CC2] |
| 197 | 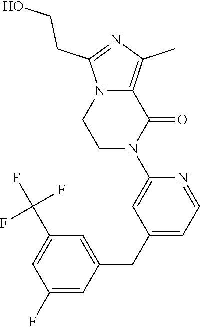 [SMILES: Cc1nc(CCO)n2c1C(=O)N(c1cc(Cc3cc(F)cc(C(F)(F)F)c3)ccn1)CC2] |
| 198 | 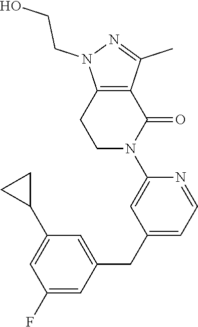 [SMILES: Cc1nn(CCO)c2c1C(=O)N(c1cc(Cc3cc(F)cc(C4CC4)c3)ccn1)CC2] |
| 199 | 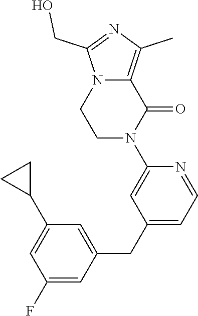 [SMILES: Cc1nc(CO)n2c1C(=O)N(c1cc(Cc3cc(F)cc(C4CC4)c3)ccn1)CC2] |
| 200 | 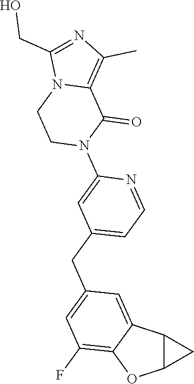 [SMILES: Cc1nc(CO)n2c1C(=O)N(c1cc(Cc3cc(F)c4c(c3)C3CC3O4)ccn1)CC2] |
| 201 | 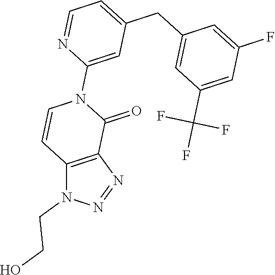 [SMILES: O=c1c2nnn(CCO)c2ccn1-c1cc(Cc2cc(F)cc(C(F)(F)F)c2)ccn1] |
| 202 | 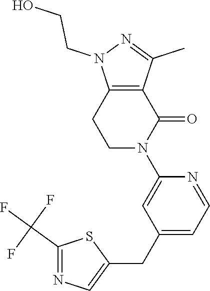 [SMILES: Cc1nn(CCO)c2c1C(=O)N(c1cc(Cc3cnc(C(F)(F)F)s3)ccn1)CC2] |
| 203 | 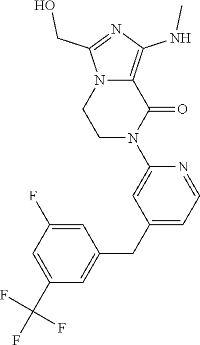 [SMILES: CNc1nc(CO)n2c1C(=O)N(c1cc(Cc3cc(F)cc(C(F)(F)F)c3)ccn1)CC2] |
| 204 | 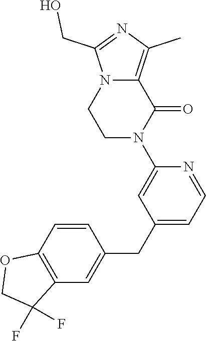 [SMILES: Cc1nc(CO)n2c1C(=O)N(c1cc(Cc3ccc4c(c3)C(F)(F)CO4)ccn1)CC2] |
| 205 | 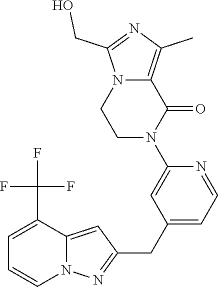 [SMILES: Cc1nc(CO)n2c1C(=O)N(c1cc(Cc3cc4c(C(F)(F)F)cccn4n3)ccn1)CC2] |
| 206 | 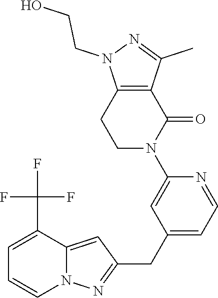 [SMILES: Cc1nn(CCO)c2c1C(=O)N(c1cc(Cc3cc4c(C(F)(F)F)cccn4n3)ccn1)CC2] |
| 207 | 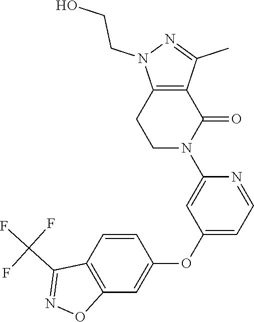 [SMILES: Cc1nn(CCO)c2c1C(=O)N(c1cc(Oc3ccc4c(C(F)(F)F)noc4c3)ccn1)CC2] |
| 208 | 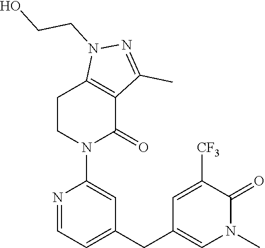 [SMILES: Cc1nn(CCO)c2c1C(=O)N(c1cc(Cc3cc(C(F)(F)F)c(=O)n(C)c3)ccn1)CC2] |
| 209 | 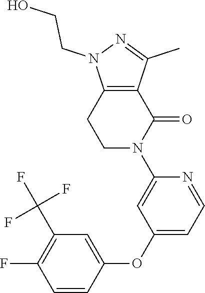 [SMILES: Cc1nn(CCO)c2c1C(=O)N(c1cc(Oc3ccc(F)c(C(F)(F)F)c3)ccn1)CC2] |
| 210 | 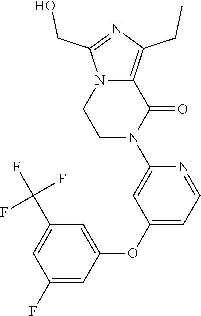 [SMILES: CCc1nc(CO)n2c1C(=O)N(c1cc(Oc3cc(F)cc(C(F)(F)F)c3)ccn1)CC2] |
| 211 | 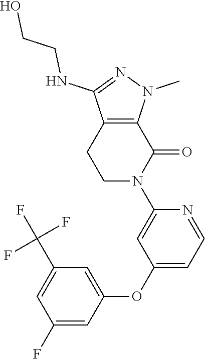 [SMILES: Cn1nc(NCCO)c2c1C(=O)N(c1cc(Oc3cc(F)cc(C(F)(F)F)c3)ccn1)CC2] |
| 212 | 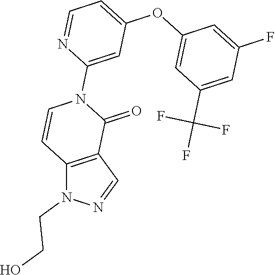 [SMILES: O=c1c2cnn(CCO)c2ccn1-c1cc(Oc2cc(F)cc(C(F)(F)F)c2)ccn1] |
| 213 | 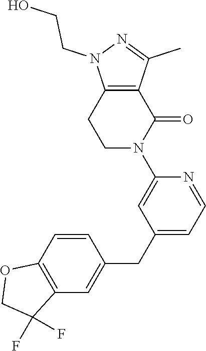 [SMILES: Cc1nn(CCO)c2c1C(=O)N(c1cc(Cc3ccc4c(c3)C(F)(F)CO4)ccn1)CC2] |
| 214 | 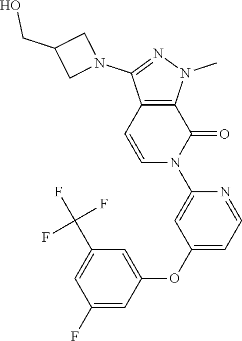 [SMILES: Cn1nc(N2CC(CO)C2)c2ccn(-c3cc(Oc4cc(F)cc(C(F)(F)F)c4)ccn3)c(=O)c21] |
| 215 | 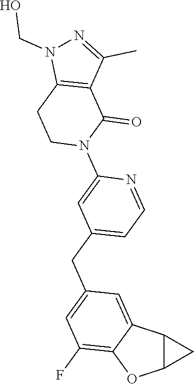 [SMILES: Cc1nn(CO)c2c1C(=O)N(c1cc(Cc3cc(F)c4c(c3)C3CC3O4)ccn1)CC2] |
| 216 | 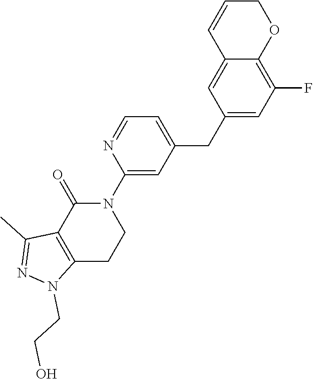 [SMILES: Cc1nn(CCO)c2c1C(=O)N(c1cc(Cc3cc(F)c4c(c3)C=CCO4)ccn1)CC2] |
| 217 | 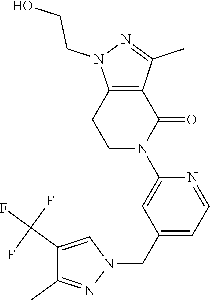 [SMILES: Cc1nn(Cc2ccnc(N3CCc4c(c(C)nn4CCO)C3=O)c2)cc1C(F)(F)F] |
| 218 | 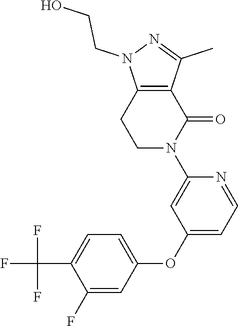 [SMILES: Cc1nn(CCO)c2c1C(=O)N(c1cc(Oc3ccc(C(F)(F)F)c(F)c3)ccn1)CC2] |
| 219 | 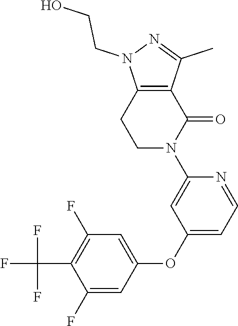 [SMILES: Cc1nn(CCO)c2c1C(=O)N(c1cc(Oc3cc(F)c(C(F)(F)F)c(F)c3)ccn1)CC2] |
| 220 | 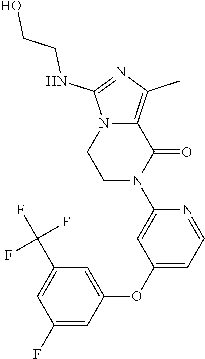 [SMILES: Cc1nc(NCCO)n2c1C(=O)N(c1cc(Oc3cc(F)cc(C(F)(F)F)c3)ccn1)CC2] |
| 221 | 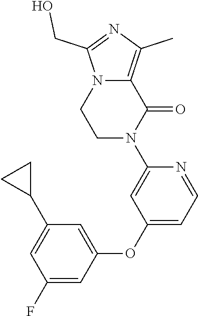 [SMILES: Cc1nc(CO)n2c1C(=O)N(c1cc(Oc3cc(F)cc(C4CC4)c3)ccn1)CC2] |
| 222 | 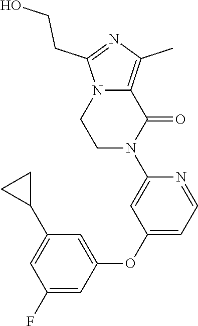 [SMILES: Cc1nc(CCO)n2c1C(=O)N(c1cc(Oc3cc(F)cc(C4CC4)c3)ccn1)CC2] |
| 223 | 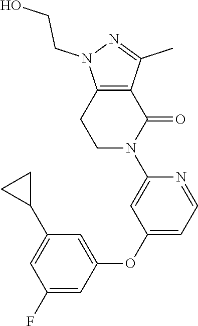 [SMILES: Cc1nn(CCO)c2c1C(=O)N(c1cc(Oc3cc(F)cc(C4CC4)c3)ccn1)CC2] |
| 224 | 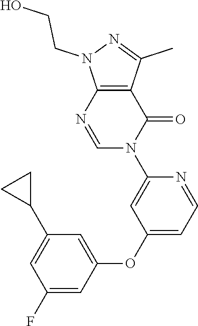 [SMILES: Cc1nn(CCO)c2ncn(-c3cc(Oc4cc(F)cc(C5CC5)c4)ccn3)c(=O)c12] |
| 225 | 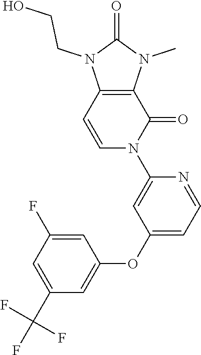 [SMILES: Cn1c(=O)n(CCO)c2ccn(-c3cc(Oc4cc(F)cc(C(F)(F)F)c4)ccn3)c(=O)c21] |
| 226 | 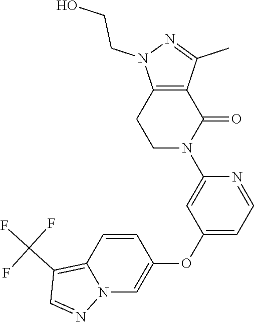 [SMILES: Cc1nn(CCO)c2c1C(=O)N(c1cc(Oc3ccc4c(C(F)(F)F)cnn4c3)ccn1)CC2] |
| 227 | 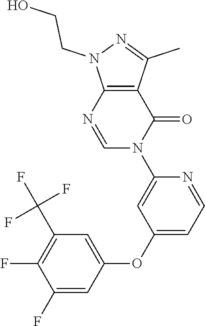 [SMILES: Cc1nn(CCO)c2ncn(-c3cc(Oc4cc(F)c(F)c(C(F)(F)F)c4)ccn3)c(=O)c12] |
| 228 | 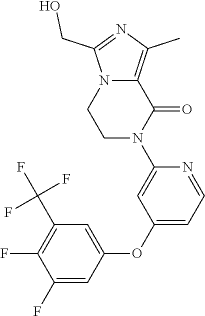 [SMILES: Cc1nc(CO)n2c1C(=O)N(c1cc(Oc3cc(F)c(F)c(C(F)(F)F)c3)ccn1)CC2] |
| 229 | 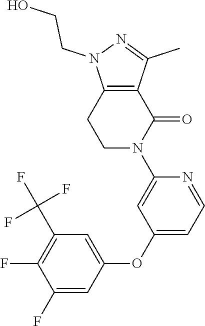 [SMILES: Cc1nn(CCO)c2c1C(=O)N(c1cc(Oc3cc(F)c(F)c(C(F)(F)F)c3)ccn1)CC2] |
| 230 | 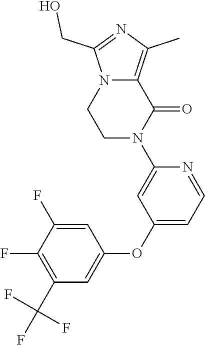 [SMILES: Cc1nc(CO)n2c1C(=O)N(c1cc(Oc3cc(F)c(F)c(C(F)(F)F)c3)ccn1)CC2] |
| 231 | 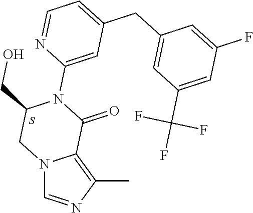 |
| 232 | 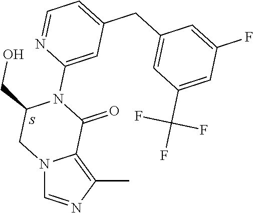 |
| 233 | 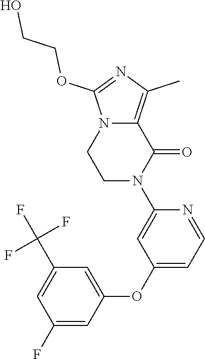 [SMILES: Cc1nc(OCCO)n2c1C(=O)N(c1cc(Oc3cc(F)cc(C(F)(F)F)c3)ccn1)CC2] |
| 234 | 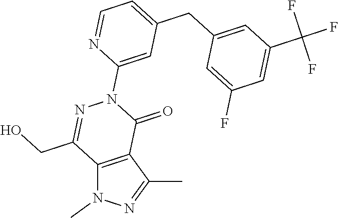 [SMILES: Cc1nn(C)c2c(CO)nn(-c3cc(Cc4cc(F)cc(C(F)(F)F)c4)ccn3)c(=O)c12] |
| 235 | 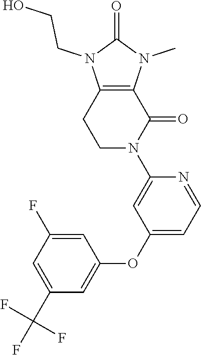 [SMILES: Cn1c2c(n(CCO)c1=O)CCN(c1cc(Oc3cc(F)cc(C(F)(F)F)c3)ccn1)C2=O] |
| 236 | 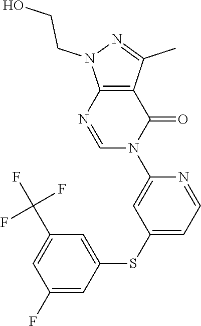 [SMILES: Cc1nn(CCO)c2ncn(-c3cc(Sc4cc(F)cc(C(F)(F)F)c4)ccn3)c(=O)c12] |
| 237 | 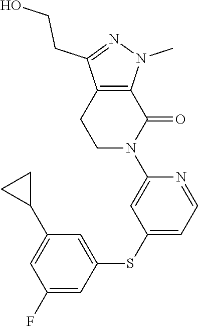 [SMILES: Cn1nc(CCO)c2c1C(=O)N(c1cc(Sc3cc(F)cc(C4CC4)c3)ccn1)CC2] |
| 238 | 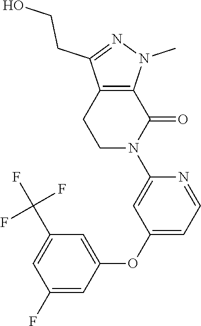 [SMILES: Cn1nc(CCO)c2c1C(=O)N(c1cc(Oc3cc(F)cc(C(F)(F)F)c3)ccn1)CC2] |
| 239 | 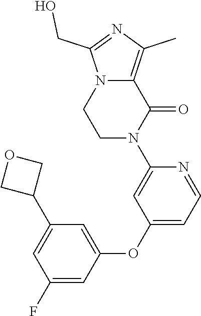 [SMILES: Cc1nc(CO)n2c1C(=O)N(c1cc(Oc3cc(F)cc(C4COC4)c3)ccn1)CC2] |
| 240 | 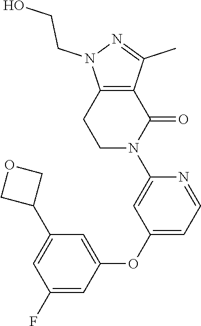 [SMILES: Cc1nn(CCO)c2c1C(=O)N(c1cc(Oc3cc(F)cc(C4COC4)c3)ccn1)CC2] |
| 241 | 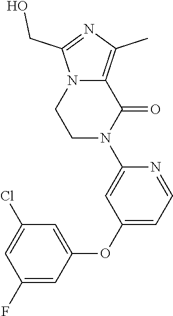 [SMILES: Cc1nc(CO)n2c1C(=O)N(c1cc(Oc3cc(F)cc(Cl)c3)ccn1)CC2] |
| 242 | 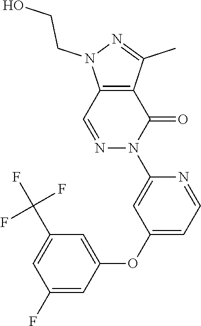 [SMILES: Cc1nn(CCO)c2cnn(-c3cc(Oc4cc(F)cc(C(F)(F)F)c4)ccn3)c(=O)c12] |
| 243 | 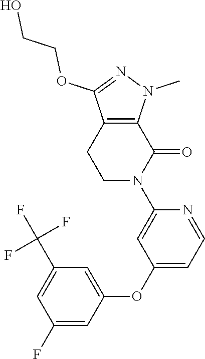 [SMILES: Cn1nc(OCCO)c2c1C(=O)N(c1cc(Oc3cc(F)cc(C(F)(F)F)c3)ccn1)CC2] |
| 244 | 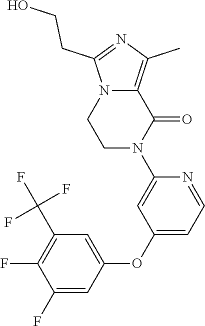 [SMILES: Cc1nc(CCO)n2c1C(=O)N(c1cc(Oc3cc(F)c(F)c(C(F)(F)F)c3)ccn1)CC2] |
| 245 | 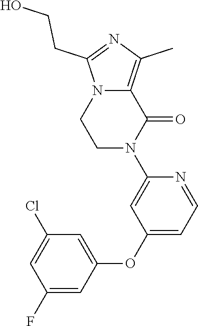 [SMILES: Cc1nc(CCO)n2c1C(=O)N(c1cc(Oc3cc(F)cc(Cl)c3)ccn1)CC2] |
| 246 | 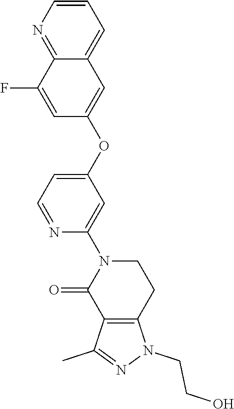 [SMILES: Cc1nn(CCO)c2c1C(=O)N(c1cc(Oc3cc(F)c4ncccc4c3)ccn1)CC2] |
| 247 | 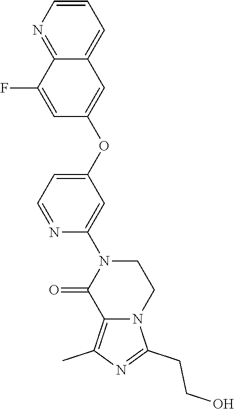 [SMILES: Cc1nc(CCO)n2c1C(=O)N(c1cc(Oc3cc(F)c4ncccc4c3)ccn1)CC2] |
| 248 | 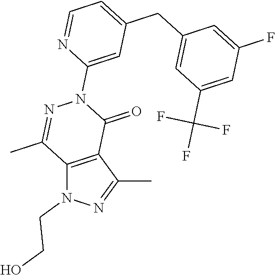 [SMILES: Cc1nn(CCO)c2c(C)nn(-c3cc(Cc4cc(F)cc(C(F)(F)F)c4)ccn3)c(=O)c12] |
| 249 | 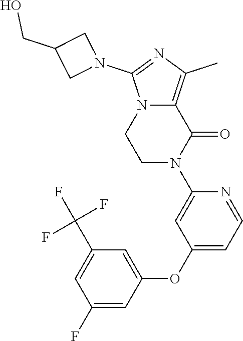 [SMILES: Cc1nc(N2CC(CO)C2)n2c1C(=O)N(c1cc(Oc3cc(F)cc(C(F)(F)F)c3)ccn1)CC2] |
| 250 | 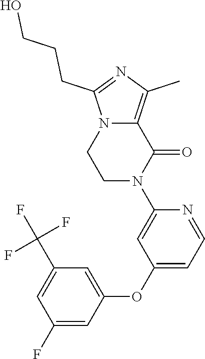 [SMILES: Cc1nc(CCCO)n2c1C(=O)N(c1cc(Oc3cc(F)cc(C(F)(F)F)c3)ccn1)CC2] |
| 251 | 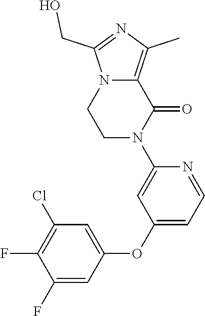 [SMILES: Cc1nc(CO)n2c1C(=O)N(c1cc(Oc3cc(F)c(F)c(Cl)c3)ccn1)CC2] |
| 252 | 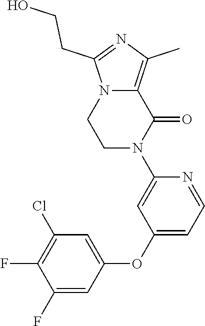 [SMILES: Cc1nc(CCO)n2c1C(=O)N(c1cc(Oc3cc(F)c(F)c(Cl)c3)ccn1)CC2] |
| 253 | 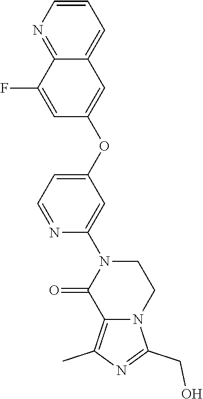 [SMILES: Cc1nc(CO)n2c1C(=O)N(c1cc(Oc3cc(F)c4ncccc4c3)ccn1)CC2] |
| 254 | 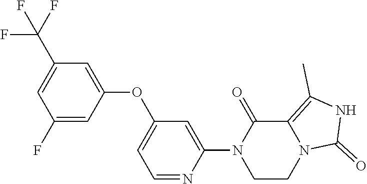 [SMILES: Cc1[nH]c(=O)n2c1C(=O)N(c1cc(Oc3cc(F)cc(C(F)(F)F)c3)ccn1)CC2] |
| 255 | 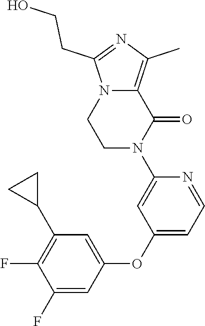 [SMILES: Cc1nc(CCO)n2c1C(=O)N(c1cc(Oc3cc(F)c(F)c(C4CC4)c3)ccn1)CC2] |
| 256 | 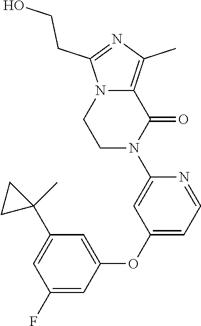 [SMILES: Cc1nc(CCO)n2c1C(=O)N(c1cc(Oc3cc(F)cc(C4(C)CC4)c3)ccn1)CC2] |
| 257 | 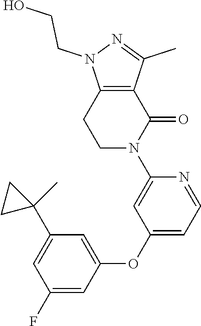 [SMILES: Cc1nn(CCO)c2c1C(=O)N(c1cc(Oc3cc(F)cc(C4(C)CC4)c3)ccn1)CC2] |
| 258 | 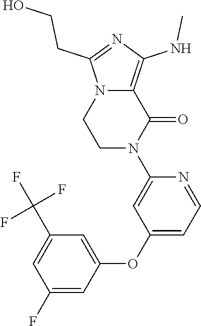 [SMILES: CNc1nc(CCO)n2c1C(=O)N(c1cc(Oc3cc(F)cc(C(F)(F)F)c3)ccn1)CC2] |
| 259 | 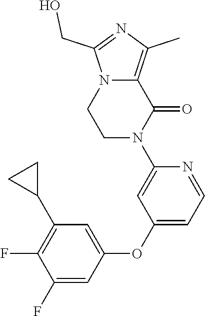 [SMILES: Cc1nc(CO)n2c1C(=O)N(c1cc(Oc3cc(F)c(F)c(C4CC4)c3)ccn1)CC2] |
| 260 | 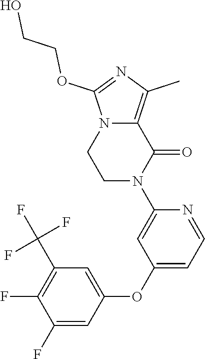 [SMILES: Cc1nc(OCCO)n2c1C(=O)N(c1cc(Oc3cc(F)c(F)c(C(F)(F)F)c3)ccn1)CC2] |
| 261 | 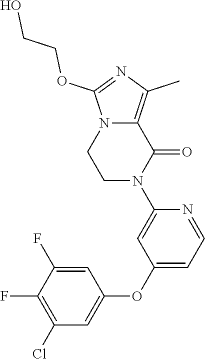 [SMILES: Cc1nc(OCCO)n2c1C(=O)N(c1cc(Oc3cc(F)c(F)c(Cl)c3)ccn1)CC2] |
| 262 | 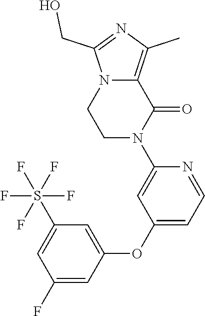 [SMILES: Cc1nc(CO)n2c1C(=O)N(c1cc(Oc3cc(F)cc(S(F)(F)(F)(F)F)c3)ccn1)CC2] |
| 263 | 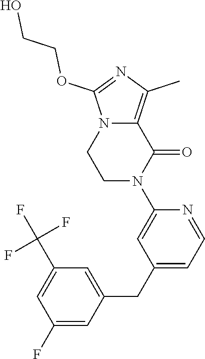 [SMILES: Cc1nc(OCCO)n2c1C(=O)N(c1cc(Cc3cc(F)cc(C(F)(F)F)c3)ccn1)CC2] |
| 264 | 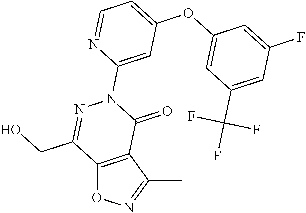 [SMILES: Cc1noc2c(CO)nn(-c3cc(Oc4cc(F)cc(C(F)(F)F)c4)ccn3)c(=O)c12] |
| 265 | 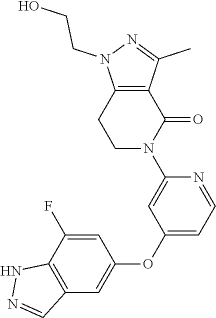 [SMILES: Cc1nn(CCO)c2c1C(=O)N(c1cc(Oc3cc(F)c4[nH]ncc4c3)ccn1)CC2] |
| 266 | 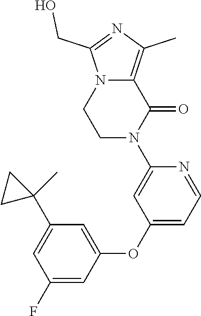 [SMILES: Cc1nc(CO)n2c1C(=O)N(c1cc(Oc3cc(F)cc(C4(C)CC4)c3)ccn1)CC2] |
| 267 | 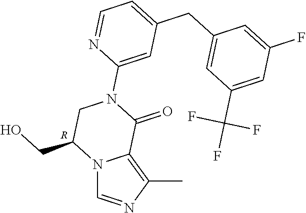 |
| 268 | 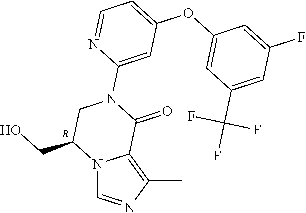 |
| 269 | 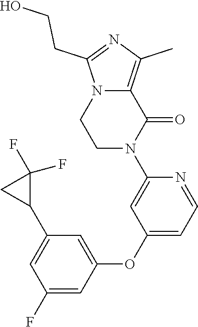 [SMILES: Cc1nc(CCO)n2c1C(=O)N(c1cc(Oc3cc(F)cc(C4CC4(F)F)c3)ccn1)CC2] |
| 270 | 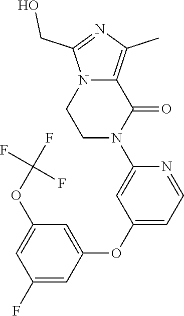 [SMILES: Cc1nc(CO)n2c1C(=O)N(c1cc(Oc3cc(F)cc(OC(F)(F)F)c3)ccn1)CC2] |
| 271 | 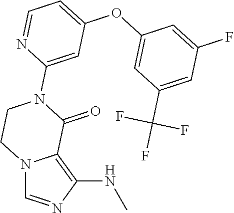 [SMILES: CNc1ncn2c1C(=O)N(c1cc(Oc3cc(F)cc(C(F)(F)F)c3)ccn1)CC2] |
| 272 | 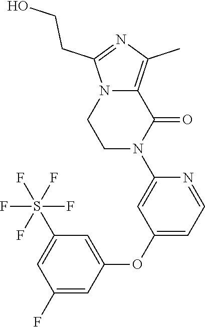 [SMILES: Cc1nc(CCO)n2c1C(=O)N(c1cc(Oc3cc(F)cc(S(F)(F)(F)(F)F)c3)ccn1)CC2] |
| 273 | 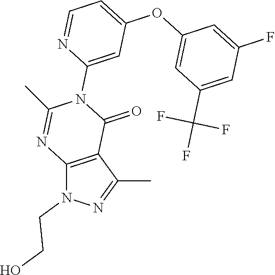 [SMILES: Cc1nn(CCO)c2nc(C)n(-c3cc(Oc4cc(F)cc(C(F)(F)F)c4)ccn3)c(=O)c12] |
| 274 | 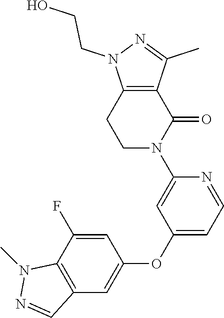 [SMILES: Cc1nn(CCO)c2c1C(=O)N(c1cc(Oc3cc(F)c4c(cnn4C)c3)ccn1)CC2] |
| 275 | 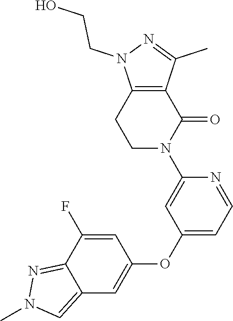 [SMILES: Cc1nn(CCO)c2c1C(=O)N(c1cc(Oc3cc(F)c4nn(C)cc4c3)ccn1)CC2] |
| 276 | 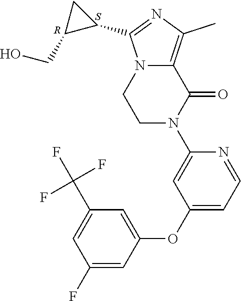 |
| 277 | 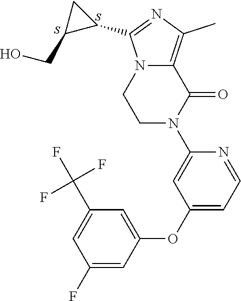 |
| 278 | 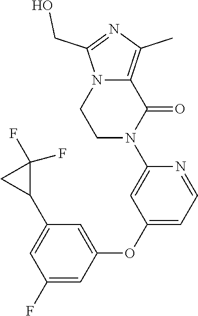 [SMILES: Cc1nc(CO)n2c1C(=O)N(c1cc(Oc3cc(F)cc(C4CC4(F)F)c3)ccn1)CC2] |
| 279 | 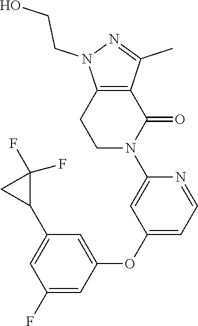 [SMILES: Cc1nn(CCO)c2c1C(=O)N(c1cc(Oc3cc(F)cc(C4CC4(F)F)c3)ccn1)CC2] |
| 280 | 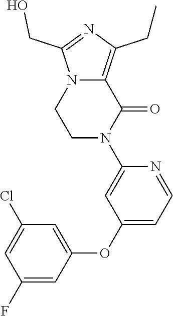 [SMILES: CCc1nc(CO)n2c1C(=O)N(c1cc(Oc3cc(F)cc(Cl)c3)ccn1)CC2] |
| 281 | 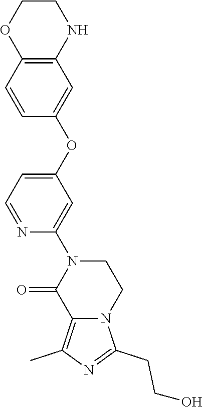 [SMILES: Cc1nc(CCO)n2c1C(=O)N(c1cc(Oc3ccc4c(c3)NCCO4)ccn1)CC2] |
| 282 | 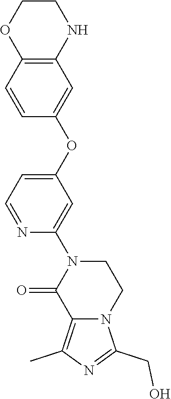 [SMILES: Cc1nc(CO)n2c1C(=O)N(c1cc(Oc3ccc4c(c3)NCCO4)ccn1)CC2] |
| 283 | 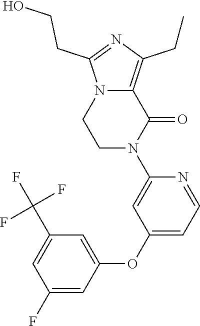 [SMILES: CCc1nc(CCO)n2c1C(=O)N(c1cc(Oc3cc(F)cc(C(F)(F)F)c3)ccn1)CC2] |
| 284 | 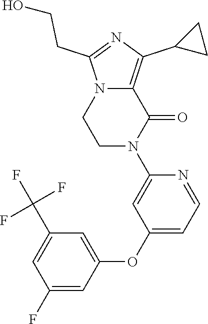 [SMILES: O=C1c2c(C3CC3)nc(CCO)n2CCN1c1cc(Oc2cc(F)cc(C(F)(F)F)c2)ccn1] |
| 285 | 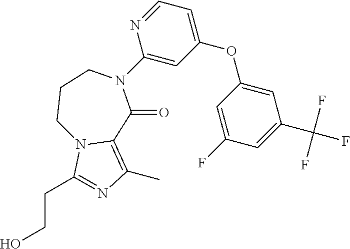 [SMILES: Cc1nc(CCO)n2c1C(=O)N(c1cc(Oc3cc(F)cc(C(F)(F)F)c3)ccn1)CCC2] |
| 286 | 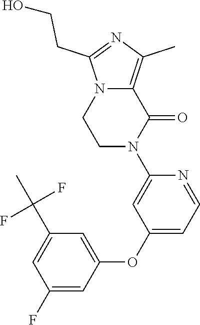 [SMILES: Cc1nc(CCO)n2c1C(=O)N(c1cc(Oc3cc(F)cc(C(C)(F)F)c3)ccn1)CC2] |
| 287 | 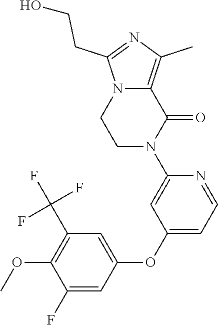 [SMILES: COc1c(F)cc(Oc2ccnc(N3CCn4c(CCO)nc(C)c4C3=O)c2)cc1C(F)(F)F] |
| 288 | 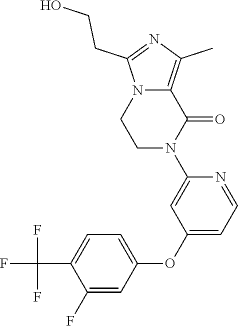 [SMILES: Cc1nc(CCO)n2c1C(=O)N(c1cc(Oc3ccc(C(F)(F)F)c(F)c3)ccn1)CC2] |
| 289 | 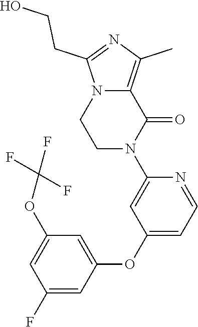 [SMILES: Cc1nc(CCO)n2c1C(=O)N(c1cc(Oc3cc(F)cc(OC(F)(F)F)c3)ccn1)CC2] |
| 290 | 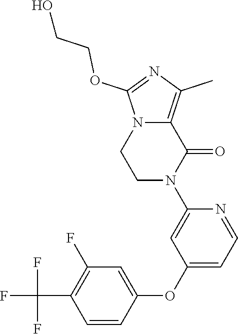 [SMILES: Cc1nc(OCCO)n2c1C(=O)N(c1cc(Oc3ccc(C(F)(F)F)c(F)c3)ccn1)CC2] |
| 291 | 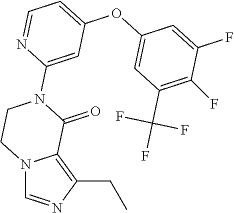 [SMILES: CCc1ncn2c1C(=O)N(c1cc(Oc3cc(F)c(F)c(C(F)(F)F)c3)ccn1)CC2] |
| 292 | 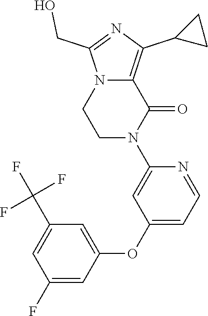 [SMILES: O=C1c2c(C3CC3)nc(CO)n2CCN1c1cc(Oc2cc(F)cc(C(F)(F)F)c2)ccn1] |
| 293 | 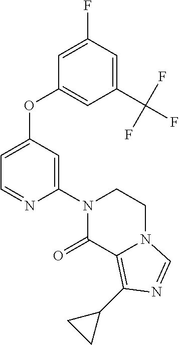 [SMILES: O=C1c2c(C3CC3)ncn2CCN1c1cc(Oc2cc(F)cc(C(F)(F)F)c2)ccn1] |
| 294 | 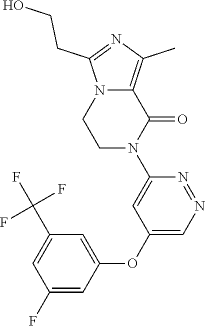 [SMILES: Cc1nc(CCO)n2c1C(=O)N(c1cc(Oc3cc(F)cc(C(F)(F)F)c3)cnn1)CC2] |
| 295 | 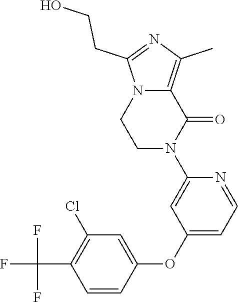 [SMILES: Cc1nc(CCO)n2c1C(=O)N(c1cc(Oc3ccc(C(F)(F)F)c(Cl)c3)ccn1)CC2] |
| 296 | 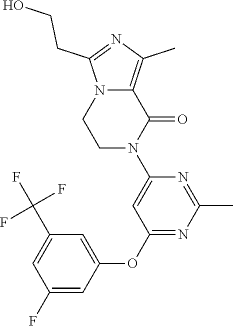 [SMILES: Cc1nc(Oc2cc(F)cc(C(F)(F)F)c2)cc(N2CCn3c(CCO)nc(C)c3C2=O)n1] |
| 297 | 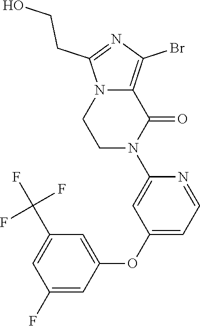 [SMILES: O=C1c2c(Br)nc(CCO)n2CCN1c1cc(Oc2cc(F)cc(C(F)(F)F)c2)ccn1] |
| 298 | 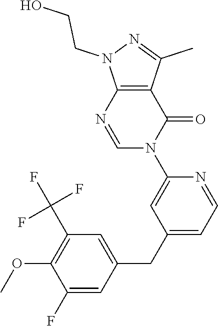 [SMILES: COc1c(F)cc(Cc2ccnc(-n3cnc4c(c(C)nn4CCO)c3=O)c2)cc1C(F)(F)F] |
| 299 | 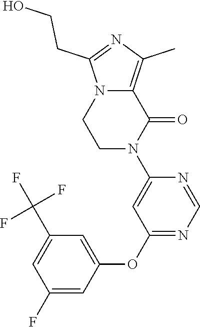 [SMILES: Cc1nc(CCO)n2c1C(=O)N(c1cc(Oc3cc(F)cc(C(F)(F)F)c3)ncn1)CC2] |
| 300 | 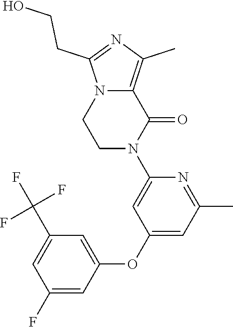 [SMILES: Cc1cc(Oc2cc(F)cc(C(F)(F)F)c2)cc(N2CCn3c(CCO)nc(C)c3C2=O)n1] |
| 301 | 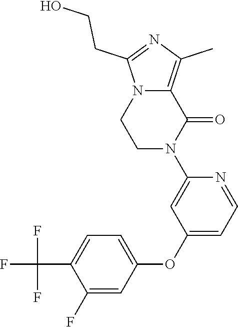 [SMILES: Cc1nc(CCO)n2c1C(=O)N(c1cc(Oc3ccc(C(F)(F)F)c(F)c3)ccn1)CC2] |
| 302 | 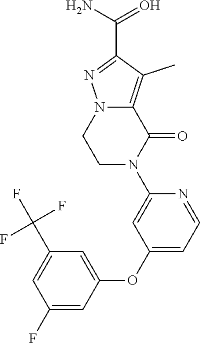 [SMILES: Cc1c(C(N)=O)nn2c1C(=O)N(c1cc(Oc3cc(F)cc(C(F)(F)F)c3)ccn1)CC2] |
| 303 | 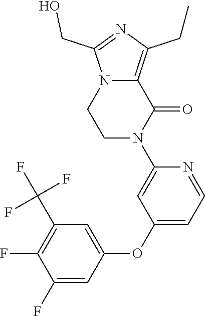 [SMILES: CCc1nc(CO)n2c1C(=O)N(c1cc(Oc3cc(F)c(F)c(C(F)(F)F)c3)ccn1)CC2] |
| 304 | 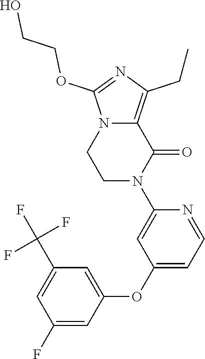 [SMILES: CCc1nc(OCCO)n2c1C(=O)N(c1cc(Oc3cc(F)cc(C(F)(F)F)c3)ccn1)CC2] |
| 305 | 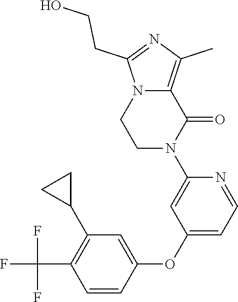 [SMILES: Cc1nc(CCO)n2c1C(=O)N(c1cc(Oc3ccc(C(F)(F)F)c(C4CC4)c3)ccn1)CC2] |
| 306 | 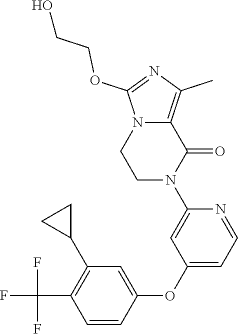 [SMILES: Cc1nc(OCCO)n2c1C(=O)N(c1cc(Oc3ccc(C(F)(F)F)c(C4CC4)c3)ccn1)CC2] |
| 307 | 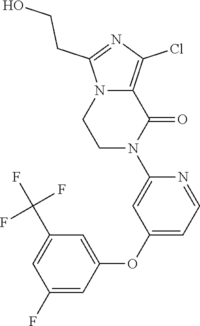 [SMILES: O=C1c2c(Cl)nc(CCO)n2CCN1c1cc(Oc2cc(F)cc(C(F)(F)F)c2)ccn1] |
| 308 | 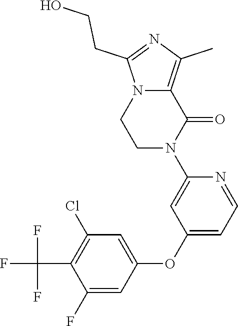 [SMILES: Cc1nc(CCO)n2c1C(=O)N(c1cc(Oc3cc(F)c(C(F)(F)F)c(Cl)c3)ccn1)CC2] |
| 309 | 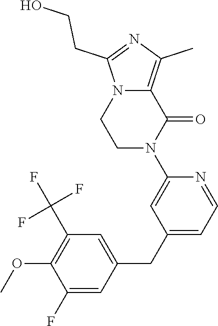 [SMILES: COc1c(F)cc(Cc2ccnc(N3CCn4c(CCO)nc(C)c4C3=O)c2)cc1C(F)(F)F] |
| 310 | 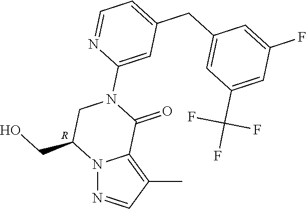 |
| 311 | 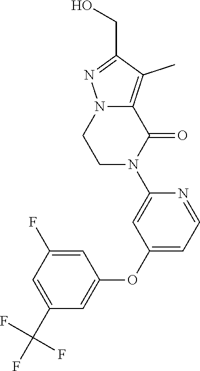 [SMILES: Cc1c(CO)nn2c1C(=O)N(c1cc(Oc3cc(F)cc(C(F)(F)F)c3)ccn1)CC2] |
| 312 | 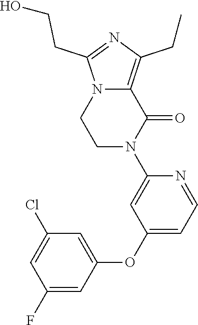 [SMILES: CCc1nc(CCO)n2c1C(=O)N(c1cc(Oc3cc(F)cc(Cl)c3)ccn1)CC2] |
| 313 | 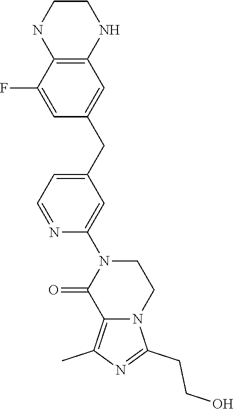 [SMILES: Cc1nc(CCO)n2c1C(=O)N(c1cc(Cc3cc(F)c4c(c3)NCCN4)ccn1)CC2] |
| 314 | 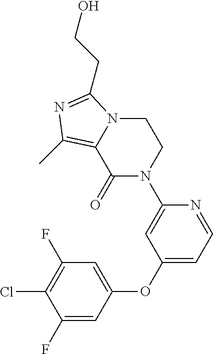 [SMILES: Cc1nc(CCO)n2c1C(=O)N(c1cc(Oc3cc(F)c(Cl)c(F)c3)ccn1)CC2] |
| 315 | 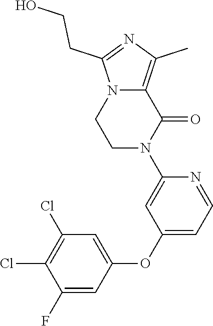 [SMILES: Cc1nc(CCO)n2c1C(=O)N(c1cc(Oc3cc(F)c(Cl)c(Cl)c3)ccn1)CC2] |
| 316 | 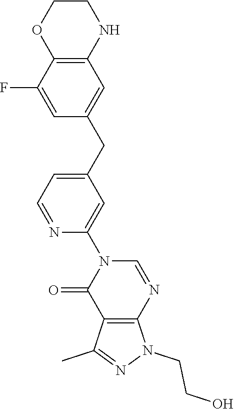 [SMILES: Cc1nn(CCO)c2ncn(-c3cc(Cc4cc(F)c5c(c4)NCCO5)ccn3)c(=O)c12] |
| 317 | 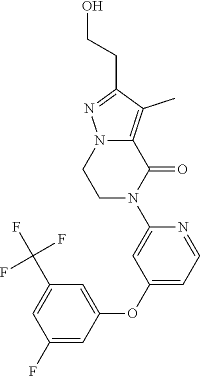 [SMILES: Cc1c(CCO)nn2c1C(=O)N(c1cc(Oc3cc(F)cc(C(F)(F)F)c3)ccn1)CC2] |
| 318 | 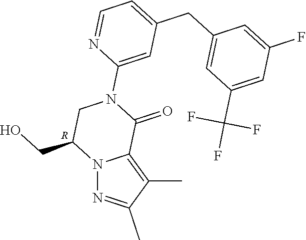 |
| 319 | 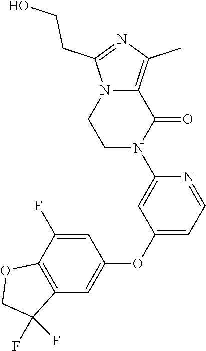 [SMILES: Cc1nc(CCO)n2c1C(=O)N(c1cc(Oc3cc(F)c4c(c3)C(F)(F)CO4)ccn1)CC2] |
| 320 | 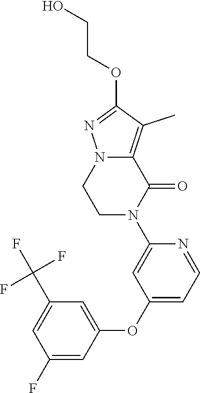 [SMILES: Cc1c(OCCO)nn2c1C(=O)N(c1cc(Oc3cc(F)cc(C(F)(F)F)c3)ccn1)CC2] |
| 321 | 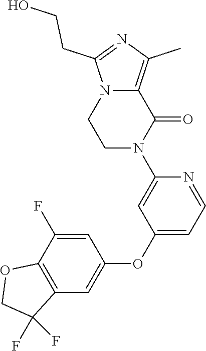 [SMILES: Cc1nc(CCO)n2c1C(=O)N(c1cc(Oc3cc(F)c4c(c3)C(F)(F)CO4)ccn1)CC2] |
| 322 | 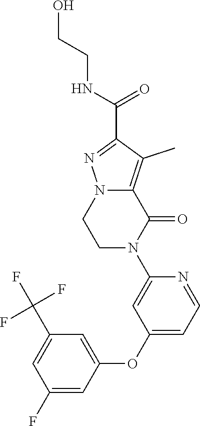 [SMILES: Cc1c(C(=O)NCCO)nn2c1C(=O)N(c1cc(Oc3cc(F)cc(C(F)(F)F)c3)ccn1)CC2] |
| 323 | 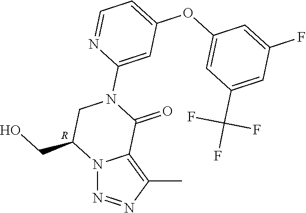 |
| 324 | 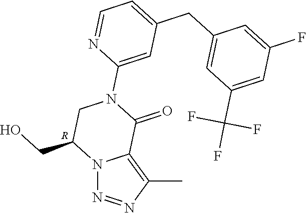 |
| 325 | 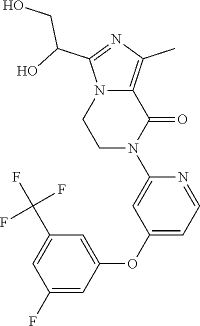 [SMILES: Cc1nc(C(O)CO)n2c1C(=O)N(c1cc(Oc3cc(F)cc(C(F)(F)F)c3)ccn1)CC2] |
| 326 | 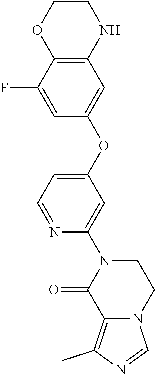 [SMILES: Cc1ncn2c1C(=O)N(c1cc(Oc3cc(F)c4c(c3)NCCO4)ccn1)CC2] |
| 327 | 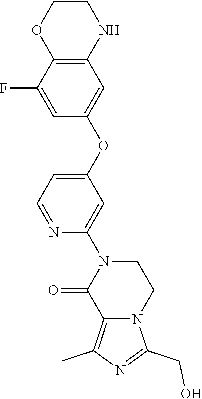 [SMILES: Cc1nc(CO)n2c1C(=O)N(c1cc(Oc3cc(F)c4c(c3)NCCO4)ccn1)CC2] |
| 328 | 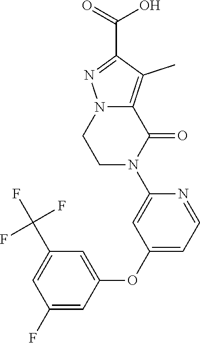 [SMILES: Cc1c(C(=O)O)nn2c1C(=O)N(c1cc(Oc3cc(F)cc(C(F)(F)F)c3)ccn1)CC2] |
| 329 | 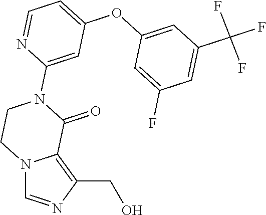 [SMILES: O=C1c2c(CO)ncn2CCN1c1cc(Oc2cc(F)cc(C(F)(F)F)c2)ccn1] |
| 330 | 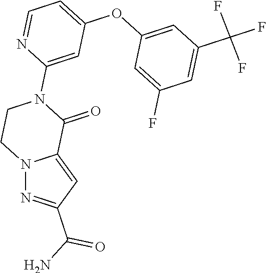 [SMILES: NC(=O)c1cc2n(n1)CCN(c1cc(Oc3cc(F)cc(C(F)(F)F)c3)ccn1)C2=O] |
| 331 | 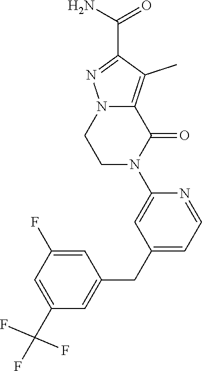 [SMILES: Cc1c(C(N)=O)nn2c1C(=O)N(c1cc(Cc3cc(F)cc(C(F)(F)F)c3)ccn1)CC2] |
| 332 | 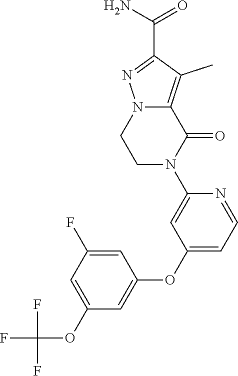 [SMILES: Cc1c(C(N)=O)nn2c1C(=O)N(c1cc(Oc3cc(F)cc(OC(F)(F)F)c3)ccn1)CC2] |
| 333 | 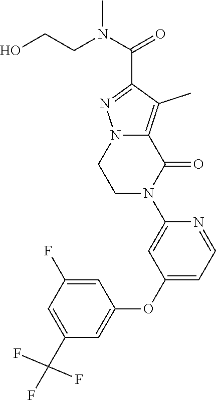 [SMILES: Cc1c(C(=O)N(C)CCO)nn2c1C(=O)N(c1cc(Oc3cc(F)cc(C(F)(F)F)c3)ccn1)CC2] |
| 334 | 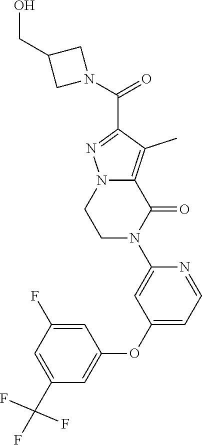 [SMILES: Cc1c(C(=O)N2CC(CO)C2)nn2c1C(=O)N(c1cc(Oc3cc(F)cc(C(F)(F)F)c3)ccn1)CC2] |
| 335 | 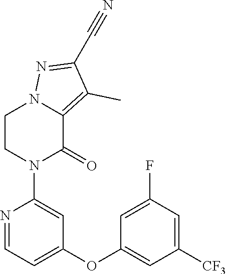 [SMILES: Cc1c(C#N)nn2c1C(=O)N(c1cc(Oc3cc(F)cc(C(F)(F)F)c3)ccn1)CC2] |
| 336 | 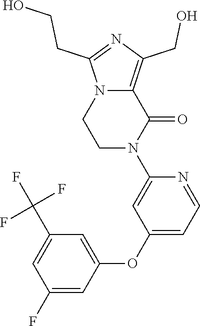 [SMILES: O=C1c2c(CO)nc(CCO)n2CCN1c1cc(Oc2cc(F)cc(C(F)(F)F)c2)ccn1] |
| 337 | 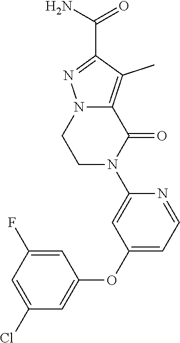 [SMILES: Cc1c(C(N)=O)nn2c1C(=O)N(c1cc(Oc3cc(F)cc(Cl)c3)ccn1)CC2] |
| 338 | 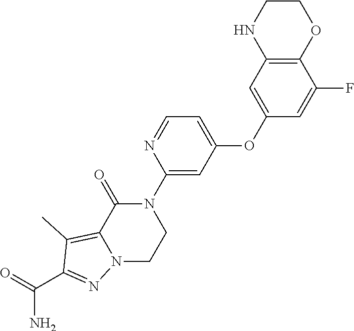 [SMILES: Cc1c(C(N)=O)nn2c1C(=O)N(c1cc(Oc3cc(F)c4c(c3)NCCO4)ccn1)CC2] |
| 339 | 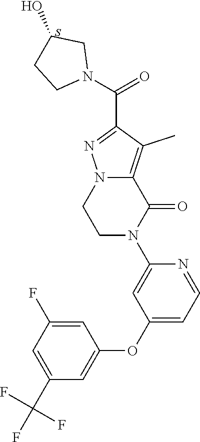 |
| 340 | 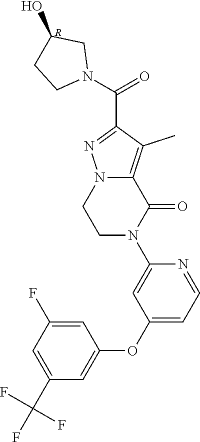 |
| 341 | 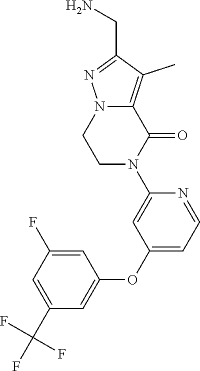 [SMILES: Cc1c(CN)nn2c1C(=O)N(c1cc(Oc3cc(F)cc(C(F)(F)F)c3)ccn1)CC2] |
| 342 | 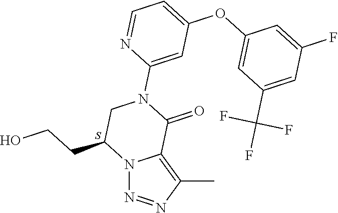 |
| 343 | 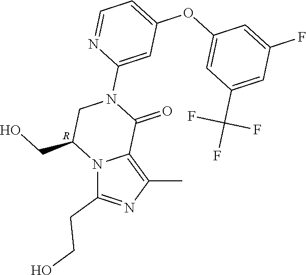 |
| 344 | 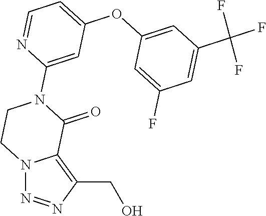 [SMILES: O=C1c2c(CO)nnn2CCN1c1cc(Oc2cc(F)cc(C(F)(F)F)c2)ccn1] |
| 345 | 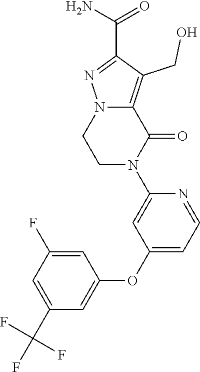 [SMILES: NC(=O)c1nn2c(c1CO)C(=O)N(c1cc(Oc3cc(F)cc(C(F)(F)F)c3)ccn1)CC2] |
| 346 | 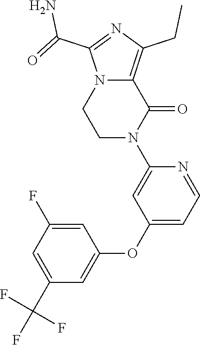 [SMILES: CCc1nc(C(N)=O)n2c1C(=O)N(c1cc(Oc3cc(F)cc(C(F)(F)F)c3)ccn1)CC2] |
| 347 | 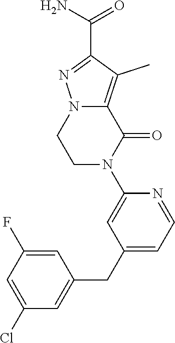 [SMILES: Cc1c(C(N)=O)nn2c1C(=O)N(c1cc(Cc3cc(F)cc(Cl)c3)ccn1)CC2] |
| 348 | 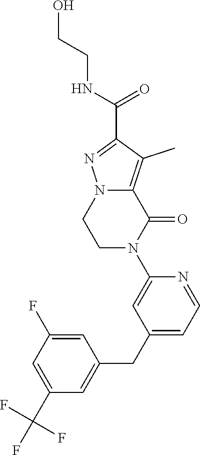 [SMILES: Cc1c(C(=O)NCCO)nn2c1C(=O)N(c1cc(Cc3cc(F)cc(C(F)(F)F)c3)ccn1)CC2] |
| 349 | 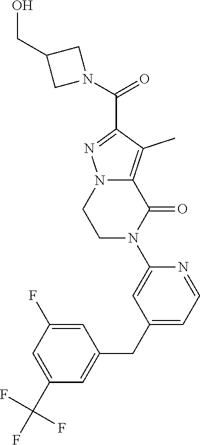 [SMILES: Cc1c(C(=O)N2CC(CO)C2)nn2c1C(=O)N(c1cc(Cc3cc(F)cc(C(F)(F)F)c3)ccn1)CC2] |
| 350 | 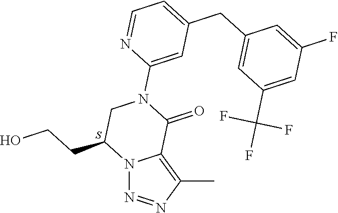 |
| 351 | 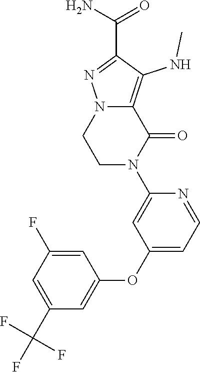 [SMILES: CNc1c(C(N)=O)nn2c1C(=O)N(c1cc(Oc3cc(F)cc(C(F)(F)F)c3)ccn1)CC2] |
| 352 | 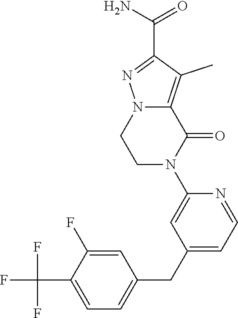 [SMILES: Cc1c(C(N)=O)nn2c1C(=O)N(c1cc(Cc3ccc(C(F)(F)F)c(F)c3)ccn1)CC2] |
| 353 | 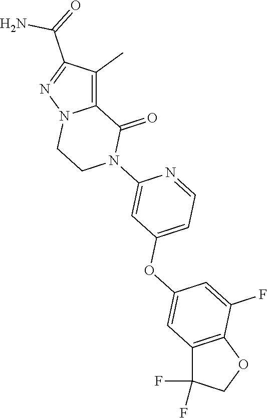 [SMILES: Cc1c(C(N)=O)nn2c1C(=O)N(c1cc(Oc3cc(F)c4c(c3)C(F)(F)CO4)ccn1)CC2] |
| 354 | 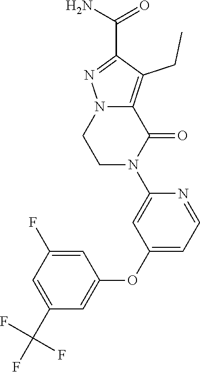 [SMILES: CCc1c(C(N)=O)nn2c1C(=O)N(c1cc(Oc3cc(F)cc(C(F)(F)F)c3)ccn1)CC2] |
| 355 | 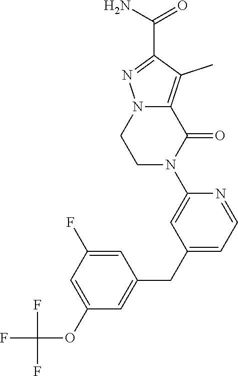 [SMILES: Cc1c(C(N)=O)nn2c1C(=O)N(c1cc(Cc3cc(F)cc(OC(F)(F)F)c3)ccn1)CC2] |
| 356 | 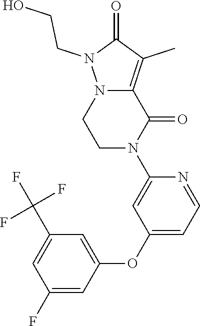 [SMILES: Cc1c2n(n(CCO)c1=O)CCN(c1cc(Oc3cc(F)cc(C(F)(F)F)c3)ccn1)C2=O] |
| 357 | 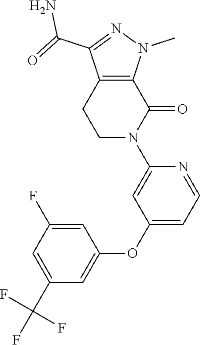 [SMILES: Cn1nc(C(N)=O)c2c1C(=O)N(c1cc(Oc3cc(F)cc(C(F)(F)F)c3)ccn1)CC2] |
| 358 | 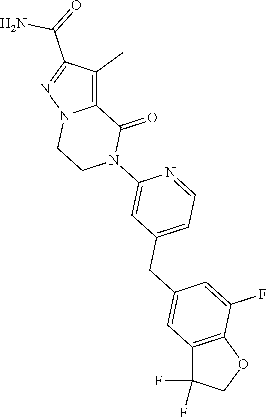 [SMILES: Cc1c(C(N)=O)nn2c1C(=O)N(c1cc(Cc3cc(F)c4c(c3)C(F)(F)CO4)ccn1)CC2] |
| 359 | 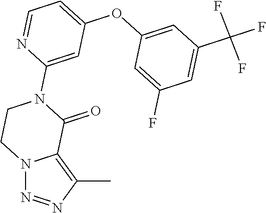 [SMILES: Cc1nnn2c1C(=O)N(c1cc(Oc3cc(F)cc(C(F)(F)F)c3)ccn1)CC2] |
| 360 | 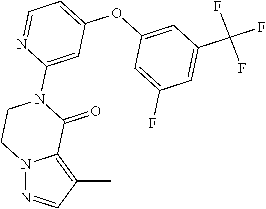 [SMILES: Cc1cnn2c1C(=O)N(c1cc(Oc3cc(F)cc(C(F)(F)F)c3)ccn1)CC2] |
| 361 | 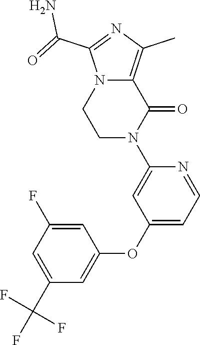 [SMILES: Cc1nc(C(N)=O)n2c1C(=O)N(c1cc(Oc3cc(F)cc(C(F)(F)F)c3)ccn1)CC2] |
| 362 | 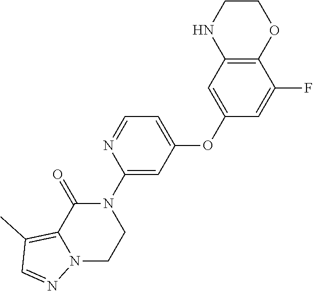 [SMILES: Cc1cnn2c1C(=O)N(c1cc(Oc3cc(F)c4c(c3)NCCO4)ccn1)CC2] |
| 363 | 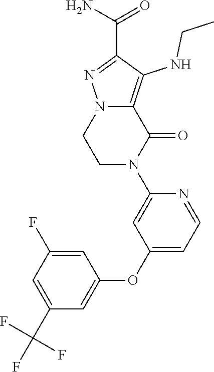 [SMILES: CCNc1c(C(N)=O)nn2c1C(=O)N(c1cc(Oc3cc(F)cc(C(F)(F)F)c3)ccn1)CC2] |
| 364 | 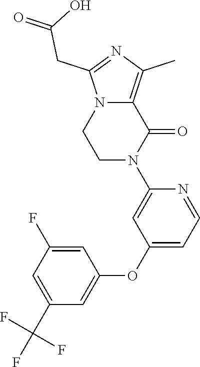 [SMILES: Cc1nc(CC(=O)O)n2c1C(=O)N(c1cc(Oc3cc(F)cc(C(F)(F)F)c3)ccn1)CC2] |
| 365 | 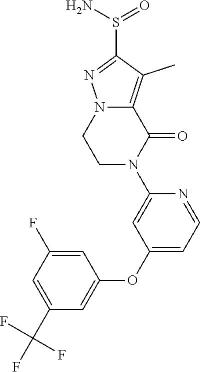 [SMILES: Cc1c(S(N)=O)nn2c1C(=O)N(c1cc(Oc3cc(F)cc(C(F)(F)F)c3)ccn1)CC2] |
| 366 | 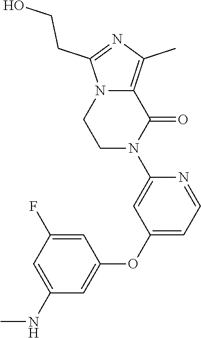 [SMILES: CNc1cc(F)cc(Oc2ccnc(N3CCn4c(CCO)nc(C)c4C3=O)c2)c1] |
| 367 | 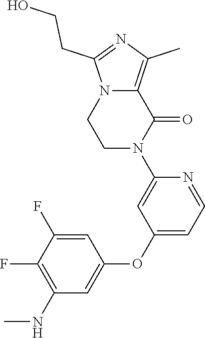 [SMILES: CNc1cc(Oc2ccnc(N3CCn4c(CCO)nc(C)c4C3=O)c2)cc(F)c1F] |

In some embodiments, the compounds described herein are agonists of GPR52.

In certain embodiments, the compounds described herein are full agonists of GPR52. In other embodiments, the compounds described herein are partial agonists of GPR52.

In some embodiments, the compounds described herein are antagonists of GPR52.

In certain embodiments, the chemical entities described herein modulate (e.g., increase) recruitment of β-arrestin to GPR52. In certain embodiments, the chemical entities described herein modulate (e.g., increase) formation of a complex (“coupling”) (e.g., a signaling complex) between β-arrestin and GPR52. The effects of the chemical entities described herein on β-arrestin signaling and associated downstream processes (e.g., those delineated above) can be assessed using conventional methods, e.g., a bioluminescence energy transfer (BRET) assay (see Examples section). By way of example, a value of β-arrestin (GLP-1R) EC50<1 μM in the aforementioned assay indicates a compound that induces β-arrestin recruitment to GLP-1R.

In some embodiments, the compounds described herein are agonists of GPR52 and increase recruitment of β-arrestin to GPR52.

In certain embodiments, the compounds described herein are full agonists of GPR52 and increase recruitment of β-arrestin to GPR52.

In certain embodiments, the compounds described herein are partial agonists of GPR52 and increase recruitment of β-arrestin to GPR52.

In one aspect, methods for modulating (e.g., agonizing, partially agonizing, or antagonizing) GPR52 activity are featured that include contacting GPR52 with a chemical entity described herein (e.g., a compound described generically or specifically herein or a pharmaceutically acceptable salt thereof or compositions containing the same). Methods include in vitro methods, e.g., contacting a sample that includes one or more cells, each comprising GPR52 with the chemical entity. Methods can also include example methods; e.g., administering the chemical entity to a subject (e.g., a human) having a disease, disorder, or condition in which modulation of GPR52 signaling is beneficial for the treatment of the underlying pathology and/or symptoms and/or progression of the disease, disorder, or condition (e.g., psychiatric disorder, e.g., schizophrenia). In some of the foregoing embodiments, said methods of modulating also include increasing β-arrestin recruitment to the GPR52.

### IV. Compositions and Administration

### Pharmaceutical Compositions

In some embodiments, the chemical entities described herein is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2— and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions can be formulated for parenteral administration, e.g., formulated for injection via the intravenous, intramuscular, sub-cutaneous, or even intraperitoneal routes. Typically, such compositions can be prepared as injectables, as either liquid solutions or suspensions; solid forms suitable for use to prepare solutions or suspensions upon the addition of a liquid prior to injection can also be prepared; and the preparations can be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and freeze-drying techniques, which yield a powder of the active ingredient, plus any additional desired ingredient from a previously sterile-filtered solution thereof.

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treated and the particular compound being employed. One skilled in the medical arts can determine the proper dosage for a particular situation. The total daily dosage may be divided and administered in portions throughout the day or by means of providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### V. Methods

The compounds of the present disclosure are believed to modulate, e.g., agonize, GPR52 activity. In some embodiments, the compounds of the present disclosure can act as GPR52 agonists on a cell. The cell can be in vitro, ex vivo, or in vivo.

Any cell that expresses GPR52 can be used in a method of the present disclosure. In some embodiments, the cell is a cell from the brain, pituitary gland, stomach, small intestine, heart, skin, tonsil, or bone marrow. In some embodiments, the cell is a brain cell, including but not limited to a neuron, such as a sensory neuron, a motor neuron, an interneuron, a pyramidal neuron, a granule cell, or a Purkinje cell, and a glial cell, e.g., an astrocyte, oligodendrocyte, or a microglial cell.

Accordingly, in some embodiments, a method of the present disclosure is a method of modulating GPR52 activity, the method comprising contacting a cell expressing GPR52 with a compound of the present disclosure or a pharmaceutically acceptable salt thereof. In some embodiments, the contacting is in vitro. In some embodiments, the contacting is ex vivo. In some embodiments, the contacting is in vivo.

In some embodiments, this disclosure provides methods of treating a disease, condition, or disorder that is causally related to GPR52 activity.

Compounds described herein can be used in the treatment of psychiatric disorders; neuropsychiatric disorders; neurodegenerative disorders; psychotic disorders; cognitive disorders; neurocognitive disorders; extrapyramidal disorders; movement disorders; motor disorders; hyperkinetic movement disorders; catatonia; mood disorders; depressive disorders; anxiety disorders; obsessive-compulsive disorder (OCD); autism spectrum disorders; depressive disorders; hypothalamic disorders; pituitary disorders; prolactin-related disorders; trauma- or stressor-related disorders; disruptive, impulse-control or conduct disorders; sleep-wake disorders; substance-related disorders; addictive disorders; behavioral disorders; hypofrontality; abnormalities in the tuberoinfundibular, mesolimbic, mesocortical, or nigrostriatal pathway; decreased activity in the striatum; cortical dysfunction; neurocognitive dysfunction or conditions or symptoms related thereto.

Compounds described herein can be used for the treatment of schizophrenia, depression, attention-deficit hyperactivity disorder (ADHD), generalised anxiety disorder, obsessive-compulsive disorder (OCD), panic disorder, bipolar disorder, addiction/impulse-control disorders, autism spectrum disorders, psychosis, anhedonia, agitation, Alzheimer's disease, Parkinson's disease, Huntington's disease, vascular dementia, Lewy body disease, frontotemporal dementia, Tourette's syndrome, hyperprolactinemia, pituitary adenoma, prolactinoma, craniopharyngioma, Cushing's disease, diabetes insipidus, non-functioning tumours, obesity, posttraumatic stress disorder (PTSD), akathisia and associated movements, athetosis, ataxia, ballismus, hemiballismus, chorea, choreoathetosis, dyskinesia, tardive dyskinesia, neuroleptic-induced dyskinesia, myoclonus, mirror movement disorder, paroxysmal kinesigenic dyskinesia, restless legs syndrome, spasms, stereotypic movement disorder, sterotypy, Tic disorder, tremor, Wilson's disease, schizotypal personality disorder, delusional disorder, brief psychotic disorder, schizophreniform disorder, schizoaffective disorder, substance- or medication-induced psychotic disorder, delusions, hallucinations, disorganized thinking, grossly disorganized or abnormal motor behavior, catatonia, major depressive disorder, bipolar I disorder, bipolar II disorder, cyclothymic disorder, substance- or medication-induced bipolar and related disorders, bipolar and related disorders due to another medical condition, separation anxiety disorder, selective mutism, specific phobia, social anxiety disorder, panic disorder, agoraphobia, generalized anxiety disorder, substance- or medication-induced anxiety disorder, anxiety disorders due to another medical condition, delirium, major neurocognitive disorder, minor neurocognitive disorder, amnesia, dementia, developmental coordination disorder, stereotypic movement disorder, a post-stroke effect, dentatorubral-pallidoluysian atrophy, diminished emotional expression, avolition, alogia and asociality.

Compounds described herein can be used for the treatment of schizophrenia, depression, attention-deficit hyperactivity disorder (ADHD), generalised anxiety disorder, obsessive-compulsive disorder (OCD), panic disorder, bipolar disorder, addiction/impulse-control disorders, autism spectrum disorders, psychosis, neurocognitive disorder, delirium, anhedonia, agitation, Alzheimer's disease, Parkinson's disease, Huntington's disease, vascular dementia, Lewy body disease, frontotemporal dementia, Tourette's syndrome, hyperprolactinemia, obesity, and posttraumatic stress disorder (PTSD). Compounds of the present invention may be used in the treatment of schizophrenia.

In some embodiments, methods of treatment of a disease, disorder, or condition are featured, in which modulation of GPR52 activity is beneficial for the treatment of the underlying pathology and/or symptoms and/or progression of the disease, disorder, or condition. The methods include administering to a subject in need of such treatment an effective amount of a chemical entity described herein (e.g., a compound described generically or specifically herein, a pharmaceutically acceptable salt thereof, or compositions containing the same).

While not wishing to be bound by theory, it is believed that the expression and functional profile of GPR52 across mesocortical and mesolimbic pathways renders it a relevant target for the treatment of a number of psychiatric disorders including, but not limited to: schizophrenia, bipolar disorder, addiction, major depressive disorder and autism spectrum disorder. Modulation of GPR52 is also believed to provide symptomatic relief in neurodegenerative diseases, e.g., Parkinson's disease, Alzheimer's disease and Huntington's disease; through addressing psychosis, motor symptoms, anhedonia and agitation.

Other non-limiting examples of such diseases, disorders, and conditions include neuropsychiatric disorders; neurodegenerative disorders; psychotic disorders; cognitive disorders; neurocognitive disorders; extrapyramidal disorders; movement disorders; motor disorders; hyperkinetic movement disorders; catatonia; mood disorders; depressive disorders; anxiety disorders; obsessive-compulsive disorder (OCD); autism spectrum disorders; hypothalamic disorders; pituitary disorders; prolactin-related disorders; trauma- or stressor-related disorders; disruptive, impulse-control or conduct disorders; sleep-wake disorders; substance-related disorders; addictive disorders; behavioural disorders; hypofrontality; abnormalities in the tuberoinfundibular, mesolimbic, mesocortical, or nigrostriatal pathways; decreased activity in the striatum; cortical dysfunction; and neurocognitive dysfunction, and related conditions or symptoms

In certain embodiments, the disease, disorder, or condition is selected from the group consisting of schizophrenia, depression, attention-deficit hyperactivity disorder (ADHD), generalised anxiety disorder, OCD, panic disorder, bipolar disorder, addiction/impulse-control disorders, autism spectrum disorders, psychosis, anhedonia, agitation, Alzheimer's disease, Parkinson's disease, Huntington's disease, vascular dementia, Lewy body disease, frontotemporal dementia, Tourette's syndrome, hyperprolactinemia, pituitary adenoma, prolactinoma, craniopharyngioma, Cushing's disease, diabetes insipidus, non-functioning tumors, obesity, posttraumatic stress disorder (PTSD), akathisia, athetosis, ataxia, ballismus, hemiballismus, chorea, choreoathetosis, dyskinesia, tardive dyskinesia, neuroleptic-induced dyskinesia, myoclonus, mirror movement disorder, paroxysmal kinesigenic dyskinesia, restless legs syndrome, spasms, stereotypic movement disorder, tic disorder, tremor, Wilson's disease, schizotypal personality disorder, delusional disorder, brief psychotic disorder, schizophreniform disorder, schizoaffective disorder, substance- or medication-induced psychotic disorder, delusions, hallucinations, disorganized thinking, grossly disorganized or abnormal motor behavior, major depressive disorder, bipolar I disorder, bipolar II disorder, cyclothymic disorder, substance- or medication-induced bipolar and related disorders, bipolar and related disorders due to another medical condition, separation anxiety disorder, selective mutism, specific phobia, social anxiety disorder, agoraphobia, anxiety disorders due to another medical condition, major neurocognitive disorder, minor neurocognitive disorder, amnesia, dementia, developmental coordination disorder, post-stroke effects, dentatorubral-pallidoluysian atrophy, diminished emotional expression, avolition, alogia, and asociality.

In certain embodiments, the disease, disorder, or condition is schizophrenia.

The methods can further include administering one or more other therapeutic agents (e.g., in combination with a chemical entity described herein).

The methods can further include identifying the subject in need of the methods of treatment described herein.

In some embodiments, the subject is a human subject, e.g., a patient, e.g., a human patient.

In some embodiments, the subject has, or previously determined to have, a decrease in GPR52 activity; e.g., a level of GPR52 activity that is lower, e.g., than a control level of said activity.

In some embodiments, the methods further include identifying, e.g., from a biological sample obtained from the subject, that the subject has said decrease in activity.

Accordingly, the methods can further include determining whether a subject (e.g., a human subject) is eligible or a candidate for treatment with a compound of Formula (J) and/or (I) or a pharmaceutically acceptable salt thereof; or a pharmaceutical composition thereof.

In some embodiments, the methods can further include evaluating the response of a subject (e.g., a human subject) to a compound of Formula (J) and/or (I) or a pharmaceutically acceptable salt thereof; or a pharmaceutical composition thereof.

### Combinations

The disclosure also features combination therapy for treating a disease or a disorder described herein. In some embodiments, the combination therapy comprises administering at least one compound described herein in combination with one or more other pharmaceutically active agents for treating cancer or other disorders mediated by menin/MLL. In some embodiments, the combination therapy comprises administering at least one compound of the present invention in combination with one or more other pharmaceutically active agents, such as for the treatment of cancer. For example, in the field of medical oncology it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents. For example, a surgery may be open surgery or minimally invasive surgery. Compounds of Formula (J) and/or (I), or pharmaceutically acceptable salts or solvates thereof, therefore may also be useful as adjuvants to cancer treatment, that is, they can be used in combination with one or more additional therapies or therapeutic agents, for example, a chemotherapeutic agent that works by the same or by a different mechanism of action. In some embodiments, a compound of Formula (J) and/or (I), or a pharmaceutically acceptable salt thereof, can be used prior to administration of an additional therapeutic agent or additional therapy. For example, a subject in need thereof can be administered one or more doses of a compound of Formula (J) and/or (I), or a pharmaceutically acceptable salt thereof, for a period of time and then undergo at least partial resection of the tumor. In some embodiments, the treatment with one or more doses of a compound of Formula (J) and/or (I), or a pharmaceutically acceptable salt thereof, reduces the size of the tumor (e.g., the tumor burden) prior to the at least partial resection of the tumor. In some embodiments, a subject in need thereof can be administered one or more doses of a compound of Formula (J) and/or (I), or a pharmaceutically acceptable salt thereof, for a period of time and under one or more rounds of radiation therapy. In some embodiments, the treatment with one or more doses of a compound of Formula (J) and/or (I), or a pharmaceutically acceptable salt thereof, reduces the size of the tumor (e.g., the tumor burden) prior to the one or more rounds of radiation therapy.

One or more additional pharmaceutical agents, for example, chemotherapeutics, anti-inflammatory agents, steroids, immunosuppressants, immune-oncology agents, metabolic enzyme inhibitors, chemokine receptor inhibitors, phosphatase inhibitors, and statins as well as targeted therapies can be used in combination with the compounds of the present disclosure for treatment of CDK2-associated diseases, disorders or conditions. The one or more additional pharmaceutical agents can be administered to a patient simultaneously or sequentially.

### VI. Examples

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following Scheme 1, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

### A. General Synthesis Methods

The compounds of the present disclosure can be prepared employing the following general methods and using procedures described in detail in the examples. The reaction conditions referred to are illustrative and non-limiting, for example one skilled in the art may use a diverse range of synthetic methods to synthesise the desired compounds such as but not limited to methods described in literature (for example but not limited to March's Advanced Organic Chemistry: Reactions, Mechanisms, and Structure, 8th Edition or Larock's Comprehensive Organic Transformations: Comprehensive Organic Transformations: A Guide to Functional Group Preparations). Compounds of formula 1, as described above, can be prepared by synthetic strategies outlined below, wherein the definitions above apply.

### 1. Preparation of Bicyclic Precursors

Scheme 1 illustrates the formation of bicycle G3 from G1 where PG represents H or a suitable protecting group including but not limited to benzyl carbamate (Cbz). Methods to form the C—N bond of G2 will be apparent to those skilled in the art, but include for example conversion via a Mitsunobu coupling between imidazole G1 and the hydroxy alkylamine G1′. Such coupling would be facilitated by use of a dialkyl azodicarboxylate such as but not limited to DEAD, DIAD, TMAD, ADDP and a phosphine such as but not limited to PPh₃ or P(n-Bu)₃. If used, deprotection of the protecting group PG will be known to those skilled in the art (for example Greene's Protective Groups in Organic Synthesis, 6th Edition). Cyclisation of G2 to G3 may be facilitated by stirring with or without heat and treatment with a base such as but not limited to triethylamine, Na₂CO₃ or K₂CO₃.

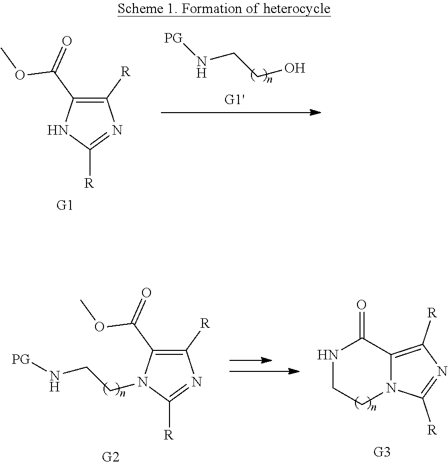

### 2. Preparation of Halo Heteroarene Precursors

Scheme 2 illustrates the formation of a carbon-carbon bond using any suitable coupling reaction, such as a Negishi cross-coupling between G4 and organozinc compound G5 where X may be a halogen or other suitable group. Methods to form the C—C bond will be apparent to those skilled in the art, but include for example treatment with a Ni- or Pd-metal catalyst such as but not limited to Ni(PPh₃)₄, Ni(acac)₂, Ni(COD)₂, Pd₂(dba)₃, PdCl₂(dppf)·DCM, Pd(PPh₃)₄ and a base such as but not limited to NaOH, NaOMe, NaOt-Bu, LiHMDS, K₂CO₃ or Cs₂CO₃.

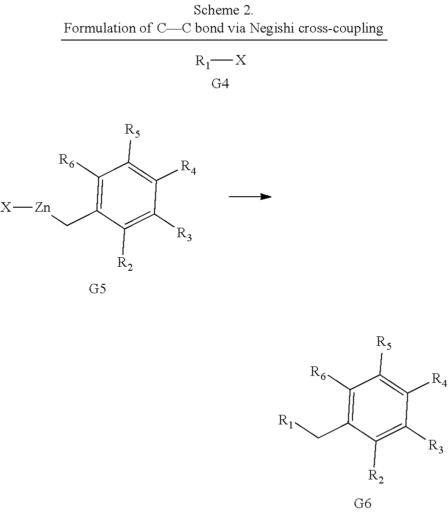

Alternatively, formation of the carbon-carbon bond may be achieved via Suzuki coupling with the relevant organoboron compound G7 and alkyl halide G8 (Scheme 3).

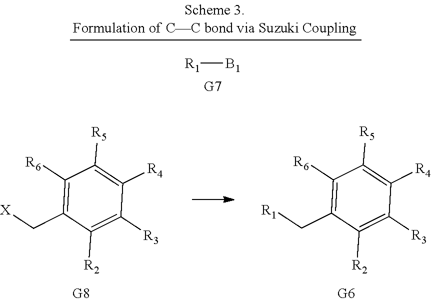

The groups R₁B₁ and X are chosen to be suitable for the coupling reaction employed. For example, in the case of a Suzuki coupling reaction, (X) may be a halogen, triflate, tosylate or other suitable group and B₁ represents a suitable boron compound such as a boronic acid or boronic ester. Examples of B₁ that can be used in the Suzuki coupling include, but are not limited to:

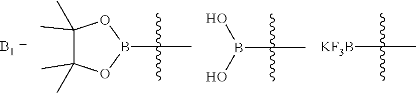

The types of R₁B₁ compounds that can be used in the Suzuki coupling include, but are not limited to boronic acid adducts shown in Table 1.

TABLE 1
Exemplary boronic acid adducts

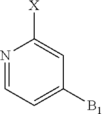

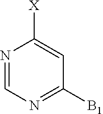

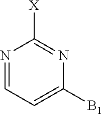

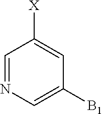

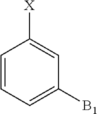

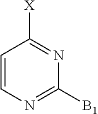

Methods to carry out the Suzuki reaction will be apparent to those skilled in the art, but include for example conversion via the use of a metal catalyst such as but not limited to PdCl₂(dppf)·DCM, Pd(PPh₃)₄ and base such as but not limited to NaOH, NaOMe, NaOt-Bu, LiHMDS, K₂CO₃ or Cs₂CO₃. In addition to Scheme 3, the position of (X) and (B₁) can be reversed as shown in Scheme 4 to give the same final compound G6.

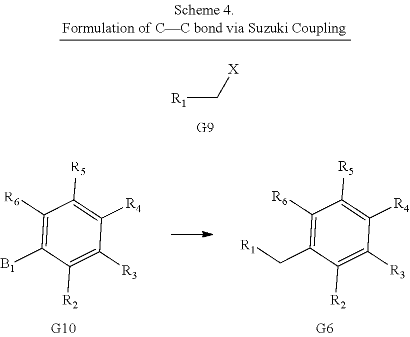

### 3. Preparation of Aryl Amines

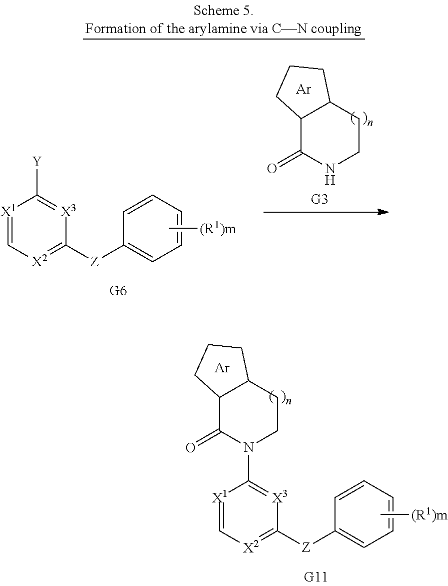

Scheme 5 illustrates the formation of a carbon-nitrogen bond by coupling of the relevant aryl halide G6, with a secondary amide or amine such as G3. The group Y is chosen to be suitable for the coupling reaction employed. Methods to form such aryl-amines will be apparent to those skilled in the art, but include for example conversion via an Ullmann coupling by treatment with a suitable Cu salt such as but not limited to CuI, Cu₂O, Cu(OAc)₂, CuBr; diamine ligand such as but not limited to DMEDA and a suitable base such as but not limited to K₂CO₃, Et₃N or pyridine. Alternatively, the conversion may be facilitated via a Buchwald-Hartwig coupling by treatment with a palladium catalyst complexed with a chelating ligand such as but not limited to Pd (OAc)₂, Pd₂(dba)₃, PdCl₂·dppf₂, XantPhos-PdG₃, Ruphos-Pd G3 and a base such as but not limited to NaOt-Bu, LiHMDS, K₂CO₃ or Cs₂CO₃.

In some embodiments, intermediates for preparing compounds and compounds of Formula (J) and/or (I) of the present invention may be prepared according to General Reaction Schemes I through VII:

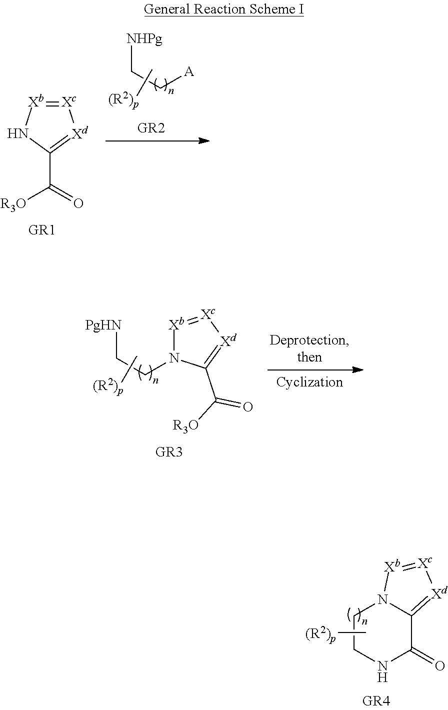

For General Reaction Scheme I, Compound GR4 is an intermediate towards preparing compounds of the disclosure, e.g., a compound of Formula (J) and/or (I). In this General Reaction Scheme, Compound GR1 is alkylated with Compound GR3 using a displacement reaction, such as a Mitsunobu reaction where A is an alcohol, in the presence of triphenyl phosphine DEAD or DIAD. Alternatively, A can represent a suitable leaving group in the presence of heat and or base in a nucleophilic displacement reaction to yield Compound GR3. Compound GR3 can then undergo a deprotection reaction to remove the nitrogen protecting group through the use of heat, acid, base, or hydrogen and a catalyst. Cyclization to Compound GR4 may be effected in situ, or through a secondary step in the presence of either a suitable Brønsted or Lewis acid, or a base.

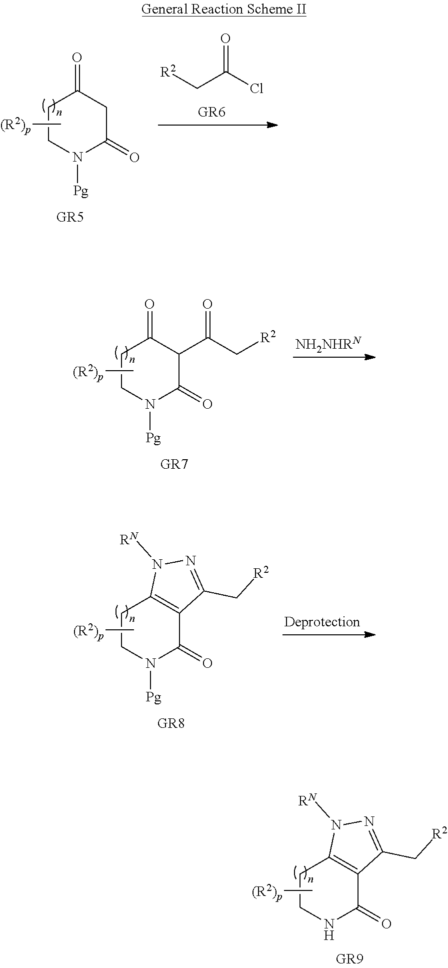

For General Reaction Scheme II, Compound GR9 is an intermediate towards preparing compounds of Formula (J) and/or (I). In this General Reaction Scheme, Compound GR5 is reacted with acid chlorides or chloroformates represented by Compound GR6 using a suitable base to provide Compound GR7. A substituted or unsubstituted hydrazine and Compound GR7 undergo dehydration to Compound GR8 in the presence of heat or a suitable acid, base, or dehydrating agent. Compound GR8 can be reacted with an appropriate acid, base, or catalyst in the presence of hydrogen or heat to yield Compound GR9.

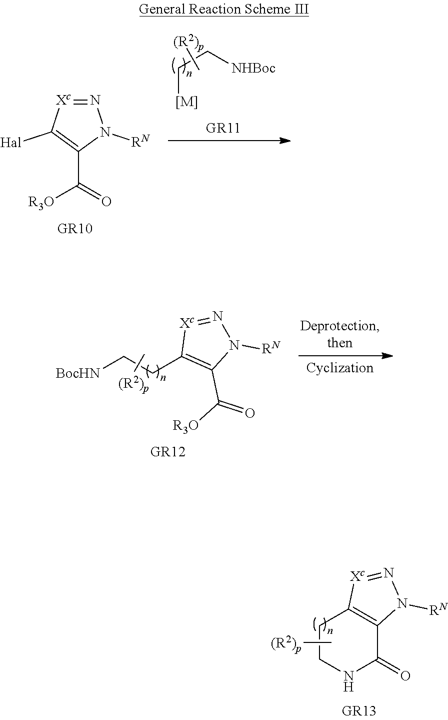

For General Reaction Scheme III, Compound GR13 is an intermediate towards preparing compounds of Formula (J) and/or (I). In this General Reaction Scheme, Compound GR10 is coupled with Compound GR11 using an appropriate transition metal catalyst and base to effect a Suzuki reaction or other such carbon-carbon bond forming cross-coupling reaction to yield Compound GR12. Removal of the nitrogen protecting group of Compound GR12 can be effected through heat, acidic, basic, or hydrogenative conditions. Suitable conditions may effect the in situ cyclization to Compound GR13. Alternatively, cyclization to Compound GR13 can be facilitated in the presence of either a suitable Brønsted or Lewis acid, or a base.

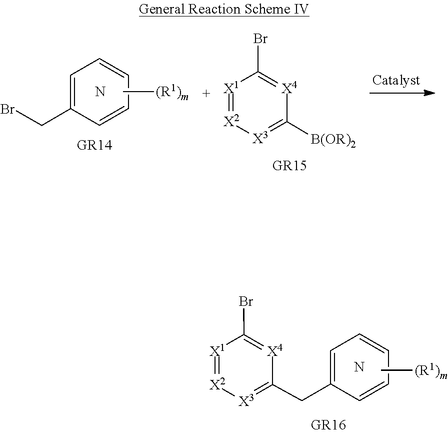

For General Reaction Scheme IV, Compound GR16 is an intermediate towards preparing compounds of Formula (J) and/or (I). In this General Reaction Scheme, Compound GR14 bearing a suitable electrophile is coupled with Compound GR15 through the use of a suitable palladium catalyst and a base to effect a Suzuki reaction or other such carbon-carbon bond forming cross-coupling reaction to yield Compound GR16.

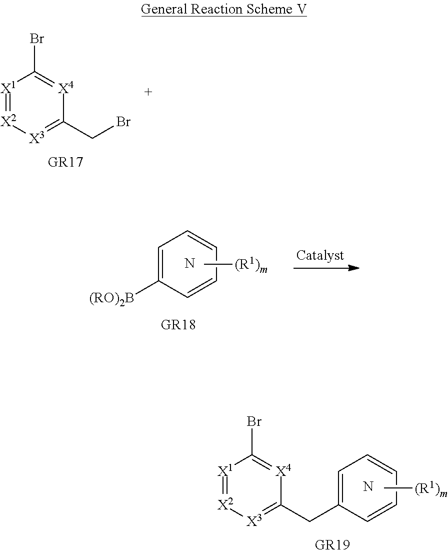

For General Reaction Scheme V, Compound GR19 is an intermediate towards preparing compounds of Formula (J) and/or (I). In this General Reaction Scheme, Compound GR17 bearing a suitable electrophile is coupled with Compound GR18 through the use of a suitable palladium catalyst and a base to effect a Suzuki reaction or other such carbon-carbon bond forming cross-coupling reaction to yield Compound GR19.

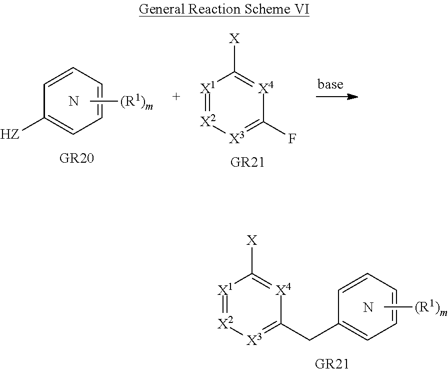

For General Reaction Scheme VI, Compound GR22 is an intermediate towards preparing compounds of Formula (J) and/or (I). In this General Reaction Scheme, Compound GR20, in the presence of a suitable base, such as cesium carbonate, potassium carbonate, triethylamine or diisopropylethylamine can be reacted with Compound GR21 through a nucleophilic aromatic substitution reaction to yield Compound GR22.

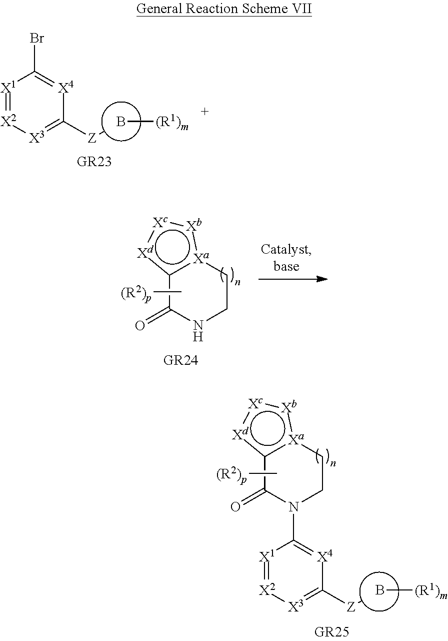

For General Reaction Scheme VII, Compound GR25 is an illustrative example of a compound of Formula (J) and/or (I). In this General Reaction Scheme, Compound GR23 is reacted with Compound GR24 in the presence of a suitable copper or palladium catalyst, base, and heat to effect an Ullmann-Goldberg, Buchwald-Hartwig, or similar carbon-nitrogen cross-coupling reaction to provide Compound GR25.

### 4. Acronyms

For convenience, many chemical moieties are represented using well known abbreviations, including but not limited to, methyl (Me), ethyl (Et), N-propyl (nPr), isopropyl (iPr), N-butyl (nBu), tert-butyl (tBu), phenyl (Ph), benzyl (Bn), methoxy (MeO), ethoxy (EtO), trimethylsilyl (TMS), and acetyl (Ac).

For convenience, many chemical compounds are represented using well known abbreviations, including but not limited to, methanol (MeOH), deuterated methanol (methanol-d4) ethanol (EtOH), isopropanol (i-PrOH), ethyl acetate (EtOAc), acetic acid (AcOH), acetonitrile (MeCN or ACN), dichloromethane (methylene chloride, DCM), trifluoroacetic acid (TFA), triflic acid (TfOH), N,N-dimethylformamide (DMF), tetrahydrofuran (THF), dimethylsulfoxide (DMSO), N-methyl-2-pyrrolidone (NMP), deuterated acetone (acetoned6), deuterated chloroform (CDCl₃), deuterated dimethylsulfoxide (DMSO-d6), 1,1′-bis(diphenylphosphino) ferrocene (dppf), triethylamine (Et₃N or TEA), (Diethylamino) sulfur trifluoride (DAST), Dess-Martin Periodinane (DMP), diisopropyl azodicarboxylate (DIAD), N,N-diisopropylethylamine (DIPEA or DIEA), 1,2-dimethylethylenediamine (DMEDA), 1,1′-bis(diphenylphosphino) ferrocene dichloropalladium (II) (PdCl₂(dppf)), palladium (II) acetate Pd(OAc)₂, Tricyclohexylphosphine (PCy₃), dichlorobis(triphenylphosphine) palladium (II) (PdCl₂(PPh₃)₂), 2,4-dimethoxybenzyl (DMB), p-methoxybenzyl (PMB), petroleum ether (Pet. ether), lithium bis(trimethylsilyl)amide (LHMDS or LiHMDS), potassium bis(trimethylsilyl)amide (KHMDS), sodium bis(trimethylsilyl)amide (NaHMDS), n-butyllithium (n-BuLi), N-bromosuccinimide (NBS), N-chlorosuccinimide (NCS), trimethylaluminum (TMA), tert-Butyl hydroperoxide (TBHP).

In addition, TLC refers to thin layer chromatography.

Other abbreviations: retention time (Rt), minute(s) (min), hour(s) (h), room temperature, (RT) or (rt), concentrated (conc.), atmosphere (atm), aqueous (aq.), saturated (sat.), equivalent(s) (eq or equiv), preparative high-performance liquid chromatography (Prep HPLC).

### 5. General Experimental Details

Unless otherwise stated the following generalisations apply. ¹H NMR spectra were recorded on a Bruker Avance neo (400 MHz) or a Bruker Ultrashield Plus (400 MHz). Samples were prepared as solutions in a suitable deuterated solvent and referenced to the appropriate internal non-deuterated solvent peak or tetramethylsilane. Chemical shifts were recorded in ppm (δ) downfield of tetramethylsilane. The multiplicity of a signal is designated by the following abbreviations: s=singlet; d=doublet; t=triplet; q=quartet; dd=doublet of doublets; ddd=doublet or doublet of doublets; dt=doublet of triplets; tt=triplet of triplets; br=broad; m=multiplet; p=pentet (quintet). All observed coupling constants, J, are reported in Hertz (Hz). Exchangeable protons are not always observed.

### 6. LCMS Methods

LCMS-01 and LCMS-08, (Method-A): Instrument: Waters AQUITY UPLC H-Class with PDA detector and QDA Performance. LC Conditions: Column: X-bridge C18, 50× 2.1 mm, 2.5 micron; Column Temperature: Ambient; Auto sampler Temperature: 15° C.; Mobile Phase: Solvent A—2 mM Ammonium Acetate+0.1% Formic acid in Milli-Q H₂O, Solvent B—0.1% Formic acid in MeCN; Gradient [time (min)/solvent B in A (%)]: 0.00/5, 0.40/5, 0.80/35, 1.20/55, 2.50/100, 3.30/100, 3.31/5, 4.00/5; Flow rate: 0.55 mL/min. MS conditions: Probe: ESI capillary; Source Temperature: 120° C.; Probe Temperature: 600° C.; Capillary Voltage: 0.8 KV (+Ve and −Ve); Cone Voltage: 10 & 30 V; Mode of Ionization: +Ve and −Ve.

LCMS-08 (Method-J): Instrument: Waters AQUITY UPLC H-Class with PDA detector and QDa Performance. LC Conditions: Column: Welch Xtimate C18, 150×4.6 mm, 5 micron; Column temperature: 35° C.; Auto sampler temperature: 15° C.; Mobile Phase: Solvent A—5 mM Ammonium Bicarbonate (pH=8.00) in Milli Q H₂O, Solvent B—MeCN; Gradient [time (min)/solvent B in A (%)]: 0.00/0, 7.00/50, 9.00/100, 14.00/100, 14.01/0, 17.00/0; Flow rate: 1.0 mL/min; UV Detection: 200-500 nm. MS conditions: Probe: ESI; Mode of Ionisation: +Ve and −Ve; Cone voltage: 30 V and 10 V; Capillary voltage: 0.8 KV; Extractor Voltage: 1 V; Rf Lens: 0.1 V; Temperature of source: 120° C.; Temperature of Probe: 600° C.; Cone Gas Flow: Default; Desolvation Gas flow: Default.

LCMS-02 (Method-A): Instrument: Waters AQUITY UPLC H-Class with PDA detector and SQD Performance. LC Conditions: Column: X-bridge C18, 50×2.1 mm, 2.5 micron; Column Temperature: Ambient; Auto sampler Temperature: 15° C.; Mobile Phase: Solvent A—2 mM Ammonium Acetate+0.1% Formic acid in Milli-Q H₂O, Solvent B-0.1% Formic acid in MeCN; Gradient [time (min)/solvent B in A (%)]: 0.00/5, 0.4/5, 0.8/35, 1.20/55, 2.50/100, 3.30/100, 3.31/5, 4.00/5; Flow rate: 0.55 mL/min. MS conditions: Probe: ESI capillary; Source Temperature: 120° C.; Probe Temperature: 600° C.; Capillary Voltage: 0.8 KV (+Ve and −Ve); Cone Voltage: 10 & 30 V; Mode of Ionization: +Ve and −Ve.

LC/MS (APCI): Instrument: 1260 Infinity II LCMS. Pump: G7112B 1260 Binary pump or G7111B 1260 Quaternary pump. UV detector: G7117C 1260 DAD HS (Diode Array Detector, High Sensitivity). MS detector: G6125B InfinityLab LC/MSD (Liquid Chromatography/Mass Selective Detector). Autosampler: G7129C 1260 Vialsampler. LC conditions: Reverse phase HPLC analysis. Column: Agilent InfinityLab Poroshell, C18, 3.0×50 mm, 2.7 μm; Column temperature: 35° C.; Injection volume: 2.00 μL. Mobile phase A: 0.1% formic acid in H₂O; mobile phase B: 0.1% formic acid in MeCN. Gradient: 5-100% of solvent B in solvent A over 2.0 min, then maintained for 2.0 min. UV detection: 214 or 254 nm. MS conditions: Mass analyser: Single Quadrupole; Ion source: Multimode ESI; Drying gas temp/flow: 300° C./12 L/min; Vaporizer temp: 225° C.; Capillary voltage: 4000 V; Scan range: 100-1000 m/z; Step size: 0.20 m/z 0.1 s; Acquisition time: 5 min.

### 7. HPLC Methods

Compound purity was determined on an Agilent 1260 Infinity II analytical HPLC equipped with a G7112B binary pump, G7129C 1260 vialsampler, G7117C DAD HS UV detector (Diode Array Detector, High Sensitivity). Software: Openlab CDS, Chemstation Edition, Rev.C.01.10 using MassHunter Walkup for sample submission. LC conditions: Reverse phase HPLC analysis. Column: Agilent Zorbax Eclipse Plus, C18, 4.6×100 mm, 3.5 μm; Column temp: 35° C.; Injection volume (default): 2.00 μL. Mobile phase A: 0.1% TFA in H₂O; mobile phase B: 0.1% TFA in MeCN; flow: 1.00 mL/min; gradient: 5-100% of solvent B in solvent A over 8.0 min, then maintained for 1.0 min. UV detection: 214 or 254 nm (default reported, wavelengths between 190 and 400 nm observed).

### 8. HRMS Methods

Instrument: 1290 Infinity II HRMS equipped with G7120A 1290 high speed pump; MS detector: G6230B 6230 LC/TOF (Liquid Chromatography/Time Of Flight). Autosampler: G7129B 1290. Vialsampler. Software (Acquisition): MassHunter Workstation, LC/MS Data Acquisition for 6200 series TOF Version 10.1, Build 10.1.48 Software (Analysis): MassHunter Workstation, Qualitative Analysis, Version 10.0, Build 10.0.10305.0. LC conditions: Direct infusion TOF mass spec. Injection volume (default): 2.00 μL; mobile phase A: 0.1% formic acid in H₂O; mobile phase B: 0.1% formic acid in MeCN; flow: 0.500 mL/min; isocratic composition: 50:50 A: B. MS condition: Mass analyser: Time of Flight (TOF); ion source: Dual AJS ESI (Agilent Jet Stream ElectroSpray Ionisation); Drying gas temp/flow: 325° C./10 L/min; Sheath gas temp/flow: 400° C./12 L/min; Capillary voltage: 4000 V; Fragmentor: 90V Skimmer: 65V Oct RFV: 750V; Scan range: 100-1700 m/z; Internal reference ions: Positive Ion Mode=m/z=121.050873 & 922.009798. Processing: Mass spectra were created by averaging the scans across each peak and background subtracted against the first 10 seconds of the TIC.

### B. Intermediates

### Intermediate 1: Synthesis of 6,7-dihydroimidazo[1,5-a]pyrazin-8(5H)-one

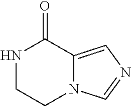

Step 1: To a stirred solution of methyl 1H-imidazole-5-carboxylate (CAS: 17325-26-7) (50.0 g, 396 mmol, 1.0 equiv) and benzyl(2-hydroxyethyl)carbamate (CAS: 77987-49-6) (115.0 g, 594.7 mmol, 1.5 equiv) in THF (500.0 mL) at −78° C. under nitrogen atmosphere. The triphenylphosphine (155.0 g, 594.6 mmol, 1.5 equiv) and DIAD (159.8 g, 792.5 mmol, 2.0 equiv) were added and the reaction mixture was stirred at room temperature for 16 h. The TLC (100% EtOAc) indicates the completion of the reaction. After completion, the reaction mixture was concentrated under reduced pressure to obtain the crude compound. The crude was purified by normal phase column chromatography (100-200 silica, gradient 0-50% EtOAc in hexane) to afford methyl 1-(2-(((benzyloxy)carbonyl)amino)ethyl)-1H-imidazole-5-carboxylate as an off-white semi-solid (60.0 g, 50% yield); LCMS-08 (Method-A): Rt 1.57 min, m/z 304.0 [M+H]⁺.

Step 2: To a stirred solution of methyl 1-(2-(((benzyloxy)carbonyl)amino)ethyl)-1H-imidazole-5-carboxylate (60.0 g, 198.5 mmol, 1.0 equiv) in methanol (600.0 mL) at room temperature, was added Pd/C (6.0 g, 10% w/w) portion-wise. The flask was filled with N₂ first and replaced with a hydrogen atmosphere using an H₂ balloon and stirring continued for 24 h at RT. The TLC (10% methanol in DCM) indicated the completion of the reaction. The reaction mixture was filtered through a Celite® bed, the bed was washed with methanol (1000 mL). The filtrate was concentrated under reduced pressure to afford the crude compound. The crude was purified by normal phase column chromatography (60-120 silica, gradient 7% MeOH in DCM) to afford 6,7-dihydroimidazo[1,5-a]pyrazin-8(5H)-one an off-white solid (16.0 g, 59% yield); ¹H NMR (400 MHz, d₆-DMSO): δ ppm 7.90 (brs, 1H), 7.83 (s, 1H), 7.43 (s, 1H), 4.18 (t, J=5.6 Hz, 2H), 3.50-3.47 (m, 2H). LCMS-02 (Method-A): Rt 0.30 min, m/z 138.2 [M+H]⁺.

### Intermediate 2: Synthesis of 1-methyl-6,7-dihydroimidazo[1,5-a]pyrazin-8(5H)-one

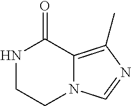

Step 1: To a stirred solution of 4-methyl-1H-imidazole-5-carboxylic acid (CAS: 1457-59-6) (10 g, 79 mmol, 1.0 equiv) in MeOH (100 mL), was added H₂SO₄ (7.7 g, 79 mmol, 1.0 equiv) at room temperature and the reaction mixture was reflux for 16 h. The reaction mixture was poured into water (200 mL) and quenched with saturated aq. NaHCO₃ (500 mL) and extracted with DCM (3× 750 mL). The organic layers were dried over Na₂SO₄ and concentrated under reduced pressure to obtain the crude compound. The crude compound was purified by manual column chromatography (60-120 silica gel, gradient 0-2% MeOH in DCM) to afford methyl 4-methyl-1H-imidazole-5-carboxylate (PRN-T-0022-A1) as a white solid (4.6 g, 41% yield), ¹H NMR (400 MHz, d₆-DMSO): δ ppm 12.42 (brs, 1H), 7.58 (s, 1H), 3.71 (s, 3H), 2.42 (s, 3H), LCMS-08 (Method-B): Rt 0.88 min, m/z 141 [M+H]⁺.

Step 2: To a stirred solution of methyl 4-methyl-1H-imidazole-5-carboxylate (PRN-T-0022-A1) (4.6 g, 32.9 mmol, 1.0 equiv) and benzyl(2-hydroxyethyl)carbamate (CAS: 77987-49-6) (9.61 g, 49.3 mmol, 1.5 equiv) in THF (46 mL) at −40° C. under nitrogen atmosphere was added triphenyl phosphine (17.2 g, 65.7 mmol, 2.0 equiv) portion wise, followed by addition of DIAD (35 mL, 65.7 mmol, 2.0 equiv) dropwise. The reaction mixture was stirred at room temperature for 14 h. After completion, as indicated by TLC (90% EtOAc in hexane) the reaction mixture was poured into ice-cold water (50 mL) and extracted with EtOAc (3×100 mL). The organic layers were dried over Na₂SO₄ and concentrated under reduced pressure to afford the crude compound. The crude compound was purified by normal phase column chromatography (60-120 silica, gradient 0-75% EtOAc in hexane) to afford methyl 1-(2-(((benzyloxy)carbonyl)amino)ethyl)-4-methyl-1H-imidazole-5-carboxylate as a white solid (2.6 g, 25% yield), ¹H NMR (400 MHz, d₆-DMSO): δ ppm 7.64 (s, 1H), 7.38-7.29 (m, 6H), 4.98 (s, 2H), 4.26 (t, J=5.6 Hz, 2H,), 3.78 (s, 3H), 3.34-3.30 (m, 2H), 2.34 (s, 3H), LCMS-01 (Method-A): Rt 1.19 min, m/z 318 [M+H]⁺.

Step 3: To a stirred solution of methyl 1-(2-(((benzyloxy)carbonyl)amino)ethyl)-4-methyl-1H-imidazole-5-carboxylate (1.0 g, 3.2 mmol, 1.0 equiv) in methanol (10.0 mL) at room temperature, was added Pd/C (1.0 g, 100% w/w) portion wise and the reaction mixture was stirred at room temperature under H₂ balloon pressure for 24 h. The TLC (15% MeOH in DCM) indicates completion of the reaction. The resulting reaction mixture was filtered through a Celite® bed, and the bed was washed with MeOH (600 mL). The filtrate was concentrated under reduced pressure to afford the crude compound. The crude compound was purified by normal phase column chromatography using (60-120 silica gel, gradient 0-10% MeOH in DCM) to afford 1-methyl-6,7-dihydroimidazo[1,5-a]pyrazin-8(5H)-one as a white solid (0.22 g, 46% yield), ¹H NMR (400 MHz, d₆-DMSO): δ ppm 7.74 (s, 1H), 7.66 (s, 1H), 4.10 (t, J=5.6 Hz, 2H,), 3.50-3.44 (m, 2H), 2.32 (s, 3H), LCMS-08 (Method-B): Rt 0.36 min, m/z 152 [M+H]⁺.

### Intermediate 3: Synthesis of 5,6,7,8-tetrahydro-9H-imidazo[1,5-a][1,4]diazepin-9-one

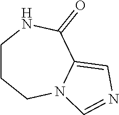 [SMILES: O=C1NCCCn2cncc21]

### General Method 1:

Step 1: To a solution of methyl 1H-imidazole-5-carboxylate (300 mg, 2.38 mmol) and benzyl N-(3-hydroxypropyl)carbamate (747 mg, 3.57 mmol) in anhydrous THF (10 mL) at −40° C. was added Ph₃P (1.25 g, 4.76 mmol). DIAD (0.94 mL, 4.76 mmol) was added dropwise into the solution and the reaction was stirred at −40° C. for 2 h then increased slowly to rt and stirred overnight. Upon completion, water was added, and the mixture was extracted with EtOAc (3 times). The combined organic layer was washed with brine, dried over MgSO₄ and concentrated in vacuo. The residue was purified by flash silica gel column chromatography eluting with EtOAc 100% to afford 600 mg of a mixture of 2 possible regioisomers methyl 1-(3-(((benzyloxy)carbonyl)amino)propyl)-1H-imidazole-5-carboxylate and methyl 1-(3-(((benzyloxy)carbonyl)amino)propyl)-1H-imidazole-4-carboxylate, which was used for the next step without further purification.

Step 2: To a solution of the above mixture (200 mg, 0.67 mmol) in MeOH (10 mL) was added 10% wt loading Pd/C (20 mg, 10% wt). The flask was filled with N₂ first and replaced with Hydrogen atmosphere using H₂ balloon. The reaction was vigorously stirred at RT for 24 h. After completion, the reaction was filtered through a pad of Celite® and washed with MeOH. The filtrate was concentrated and dissolved again in MeCN: H₂O (21 mL, 20:1 ratio) and stirred at 120° C. for 8 h. After completion, the solvent was removed and the residue was purified by flash silica gel column chromatography eluting with EtOAc: MeOH (5:2) to afford the title compound (21 mg, 21% over 2 synthetic steps). LC/MS (APCI): m/z=152.1 [M+H]⁺, Rt 0.44 min. ¹H NMR (400 MHz, DMSO-d6) δ 7.84 (s, 1H), 7.75 (d, J=1.1 Hz, 1H), 7.39 (d, J=1.1 Hz, 1H), 4.23 (t, J=6.5 Hz, 2H), 3.18-3.09 (m, 2H), 2.02 (p, J=6.4 Hz, 2H).

The following intermediates were synthesised using general method 1, using the specific reagent amounts detailed below.

### Intermediate 4: Synthesis of 3-methyl-6,7-dihydroimidazo[1,5-a]pyrazin-8 (5H)-one

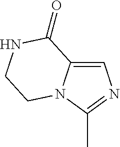

### General Method 1:

Step 1: Methyl 2-methyl-1H-imidazole-5-carboxylate (200 mg, 1.43 mmol, 1 equiv), N,Z-ethanolamine (1.5 equiv), Ph₃P (2.5 equiv), DIAD (2.5 equiv), THF (10 mL). Purification by flash column chromatography eluting with EtOAc: petroleum ether (2:1) afforded 411 mg of a mixture of 2 regioisomers methyl 1-(2-(((benzyloxy)carbonyl)amino)ethyl)-2-methyl-1H-imidazole-5-carboxylate and methyl 1-(2-(((benzyloxy)carbonyl)amino)ethyl)-2-methyl-1H-imidazole-4-carboxylate. LC/MS (APCI): m/z=318.1 [M+H]⁺, Rt 2.94 min.

Step 2: The above mixture (411 mg, 3.30 mmol), 10% Pd/C (81 mg, 20% wt), MeOH (8 mL), RT, 24 h, then MeOH (150 mL), 80° C., 48 h. Purification by flash column chromatography eluting with EtOAc: MeOH (5:2) afforded the title compound as a colorless solid (91 mg, 37% after 2 steps). LC/MS (APCI): m/z=152.1 [M+H]⁺, Rt 0.45 min, ¹H NMR (400 MHz, DMSO-d6) δ 7.80 (br s, 1H), 7.33 (s, 1H), 4.07-4.01 (m, 2H), 3.51-3.46 (m, 2H), 2.33 (s, 3H).

### Intermediate 5: Synthesis of intermediate 1-(trifluoromethyl)-6,7-dihydroimidazo[1,5-a]pyrazin-8(5H)-one

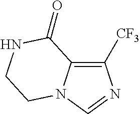 [SMILES: O=C1NCCn2cnc(C(F)(F)F)c21]

### General Method 1:

Step 1: Ethyl 4-(trifluoromethyl)-1H-imidazole-5-carboxylate (200 mg, 0.96 mmol, 1 equiv), N,Z-ethanolamine (1.5 equiv), Ph₃P (2.5 equiv), DIAD (2.5 equiv), THF (10 mL). Purification by flash column chromatography eluting with EtOAc: petroleum ether (1:1) afforded 251 mg of a mixture of 2 regioisomers ethyl 1-(2-(((benzyloxy)carbonyl)amino) ethyl)-4-(trifluoromethyl)-1H-imidazole-5-carboxylate and ethyl 1-(3-(benzyloxy)-3-oxopropyl)-5-(trifluoromethyl)-1H-imidazole-4-carboxylate. LC/MS (APCI): m/z=386.1 [M+H]⁺, Rt 3.29 min.

Step 2: The above mixture (251 mg, 0.65 mmol), 10% Pd/C (50 mg, 20 wt %), MeOH (5 mL), rt, 24 h, then MeOH (100 mL), 80° C., 24 h. Purification by flash silica gel column chromatography eluting with EtOAc: MeOH (5:2) affords the title compound as a colorless solid (72 mg, 36% after 2 steps). LC/MS (APCI): m/z=206.0 [M+H]⁺, Rt 1.58 min. ¹H NMR (400 MHz, DMSO-d6) δ 8.27 (s, 1H), 7.98 (s, 1H), 4.29-4.21 (m, 2H), 3.56-3.49 (m, 2H).

### Intermediate 6: Synthesis of intermediate 6,7-dihydro-[1,2,3]triazolo[1,5-a]pyrazin-4(5H)-one

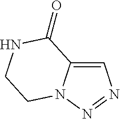

### General Method 1:

Step 1: Ethyl 1H-1,2,3-triazole-5-carboxylate (200 mg, 1.42 mmol, 1 equiv), N,Z-ethanolamine (1.5 equiv), Ph₃P (2.5 equiv), DIAD (2.5 equiv), THF (10 mL). Purification by flash silica gel column chromatography eluting with EtOAc: petroleum benzine (2:1) afforded 411 mg of a mixture of 2 regioisomers ethyl 1-(2-(((benzyloxy)carbonyl)amino) ethyl)-1H-1,2,3-triazole-5-carboxylate and ethyl 1-(2-(((benzyloxy)carbonyl)amino)ethyl)-1H-1,2,3-triazole-4-carboxylate. LC/MS (ESI): m/z=319.0 [M+H]⁺, Rt 3.49 min.

Step 2: The above mixture (411 mg, 1.29 mmol), 10% Pd/C (82 mg, 20% wt), RT, 24 h, then MeOH (200 mL), 80° C., 48 h. purified by flash silica gel column chromatography eluting with EtOAc: MeOH (5:2) affords the title compound as a colorless solid (72 mg, 37% after 2 steps). LC/MS (APCI): m/z=139.2 [M+H]⁺, Rt 0.512 min. ¹H NMR (400 MHz, DMSO-d6) δ 8.42 (s, 1H), 8.18 (s, 1H), 4.62-4.58 (m, 2H), 3.67-3.62 (m, 2H).

### Intermediate 7: Synthesis of intermediate 5-methyl-6,7-dihydroimidazo[1,5-a]pyrazin-8(5H)-one

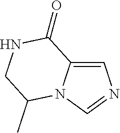

### General Method 1:

Step 1: Methyl 1H-imidazole-5-carboxylate (500 mg, 3.96 mmol, 1 equiv), benzyl (2-hydroxypropyl)carbamate (1.5 equiv), Ph₃P (2.5 equiv), DIAD (2.5 equiv), THF (20 mL). Purification by flash column chromatography eluting with EtOAc 100% afforded 826 mg of a mixture of 2 regioisomers methyl 1-(2-(((benzyloxy)carbonyl)amino)propyl)-1H-imidazole-5-carboxylate and methyl 1-(2-(((benzyloxy)carbonyl)amino)propyl)-1H-imidazole-4-carboxylate. LC/MS (APCI): m/z=318.1 [M+H]⁺, Rt 3.03 min.

Step 2: The above mixture (826 mg, 2.60 mmol), 10% Pd/C (162 mg, 20 wt %), MeOH (20 mL), RT, 24 h, then MeOH (200 mL), 80° C., 24 h. Purification by flash silica gel column chromatography eluting with EtOAc: MeOH (5:2) affords the title compound as a colorless solid (111 mg, 19% after 2 steps). LC/MS (APCI): m/z=152.1 [M+H]⁺, Rt 0.464 min. ¹H NMR (400 MHz, DMSO-d6) δ 7.91 (s, 1H), 7.87 (s, 1H), 7.44 (d, J=0.9 Hz, 1H), 4.42 (ddd, J=7.7, 6.3, 4.1 Hz, 1H), 3.55-3.47 (m, 1H), 3.24 (ddd, J=13.0, 7.9, 2.4 Hz, 1H), 1.46 (d, J=6.5 Hz, 3H).

### Intermediate 8: Synthesis of intermediate 3-(trifluoromethyl)-6,7-dihydroimidazo[1,5-a]pyrazin-8(5H)-one

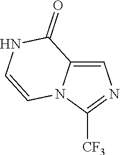 [SMILES: O=c1[nH]ccn2c(C(F)(F)F)ncc12]

Step 1: To a mixture of 2-(trifluoromethyl)-1H-imidazole-5-carboxylic acid (300 mg, 1.67 mmol) and MeOH (10 mL) was added slowly H₂SO₄ (0.45 mL, 8.3 mmol). The reaction was heated at 65° C. for 16 h. After completion, the reaction was concentrated in vacuo. The residue was neutralized by saturated solution of NaHCO₃ and extracted with EtOAc (3×100 mL). The combined organic layer was washed with brine and dried over MgSO₄ and then concentrated in vacuo to afford methyl 2-(trifluoromethyl)-1H-imidazole-5-carboxylate as a colorless solid (157 mg, 49%). The product was used for the next step without further purification. LC/MS (APCI): m/z=195.0 [M+H]⁺, RT 2.79 min. ¹H NMR (400 MHz, DMSO-d6) δ 8.12 (s, 1H), 3.75 (s, 3H).

Step 2: To a solution of methyl 2-(trifluoromethyl)-1H-imidazole-5-carboxylate (100 mg, 0.52 mmol) and N,Z-ethanolamine (151 mg, 0.77 mmol) in anhydrous THF (5 mL) at −40° C. was added Ph₃P (270 mg, 1.03 mmol). DIAD (202 μL, 1.03 mmol) was added dropwise into the solution and the reaction was stirred at −40° C. for 2 h then increased slowly to rt and stirred overnight. Upon completion, water (20 mL) was added, and the mixture was extracted with EtOAc (3×50 mL). The combined organic layer was washed with brine, dried over MgSO₄ and concentrated in vacuo. The residue was purified by flash silica gel column chromatography eluting with EtOAc:PET benzine (1:2) to afford 162 mg of an inseparable mixture of 2 regioisomers methyl 1-(2-(((benzyloxy)carbonyl)amino)ethyl)-2-(trifluoromethyl)-1H-imidazole-5-carboxylate and methyl 1-(2-(((benzyloxy)carbonyl) amino)ethyl)-2-(trifluoromethyl)-1H-imidazole-4-carboxylate). LC/MS (APCI): m/z=372.1 [M+H]⁺, Rt 3.67 min. ¹H NMR (400 MHz, CDCl₃) δ 7.74 (s, 1H), 7.39-7.27 (m, 5H), 5.05 (s, 2H), 4.61 (t, J=6.0 Hz, 2H), 3.86 (s, 3H), 3.62 (q, J=6.2 Hz, 2H).

Step 3: To a solution of the mixture from step 2 (162 mg, 0.436 mmol) in MeOH (6 mL) was added 10% Pd/C (32 mg, 20% wt). The flask was filled with N₂ first and replaced with hydrogen atmosphere using H₂ balloon. The reaction was vigorously stirred at RT for 72 h. After completion, the solvent was removed, and the residue was purified by flash silica gel column chromatography eluting with EtOAc (100%) to afford the title compound as a colorless solid (51 mg, 42% after 2 steps). LC/MS (APCI): m/z=206.0 [M+H]⁺, Rt 2.61 min. ¹H NMR (400 MHz, DMSO-d6) δ 8.25 (s, 1H), 7.62 (s, 1H), 4.33 (t, J=6.0 Hz, 2H), 3.64-3.55 (m, 2H).

### Intermediate 9: Synthesis of 1,3-dimethyl-6,7-dihydroimidazo[1,5-a]pyrazin-8(5H)-one

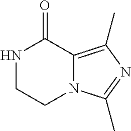

Step 1: Sodium nitrite (1.00 g, 14.9 mmol, 1.0 equiv.) solution in water (10 mL) was added dropwise over 1 h to a solution of ethyl acetoacetate (1.55 g, 11.9 mmol, 0.8 equiv) in glacial acetic acid (10 mL) at room temperature. The resulting reaction mixture was stirred for an additional 1 h at room temperature followed by the addition of 20 mL of water. After stirring for an additional 2 h, the reaction mixture was extracted with ether (3×50 mL), and successively washed with aqueous NaHCO₃ (50 mL), water (30 mL), and brine (30 mL). The organic layers were dried with Na₂SO₄ and concentrated under reduced pressure to afford the crude compound. The crude product (1.5 g) was dissolved in a mixture of acetic anhydride (2.82 mL, 59.7 mmol, 2.0 equiv), and acetic acid (10 mL) then 10% Pd/C (150 mg, 15% w/w) was added portion-wise. The hydrogenator was filled with N₂ first and replaced with hydrogen atmosphere using H_{2(g)} and stirred at 50 psi for 16 h. After completion of the reaction as indicated by TLC (40% EtOAc in hexane as a mobile phase), the reaction mixture was filtered through a celite bed and the bed was washed with EtOAc (50 mL). The filtrate was collected and concentrated under reduced pressure to afford the crude compound. The crude compound was purified by normal phase column chromatography (230-400 silica, 0-30% EtOAc in hexane) to afford ethyl 2-acetamido-3-oxobutanoate as a colourless liquid (0.20 g, 14% yield); ¹H NMR (400 MHz, d₆-DMSO): δ ppm 8.64 (d, J=7.2 Hz, 1H), 5.19 (d, J=7.2, 1H), 4.19-4.13 (m, 2H), 2.23 (s, 3H), 1.92 (s, 3H), 1.21 (t, J=7.2, 3H); LCMS-07 (Method-A): Rt 1.36 min, m/z 188.2 [M+H]⁺.

Step 2: Ammonium acetate (20.0 g, 259 mmol, 1.0 equiv) was added to a solution of ethyl 2-(acetylamino)-3-oxobutanoate (9.8 g, 52 mmol, 0.2 equiv) in acetic acid (1.0 L). The reaction mixture was heated to 120° C. for 16 h. After completion of the reaction, as indicated by TLC (70% EtOAc in hexane), the reaction mixture was filtered through a celite bed, and the celite bed was washed with EtOAc (200 mL). The filtrate was concentrated under reduced pressure to afford the crude compound. The crude compound was purified by normal phase column chromatography (100-200 silica, 0-60% EtOAc in hexane) to afford ethyl 2,4-dimethyl-1H-imidazole-5-carboxylate as a light brown solid (3.0 g, 17% yield); ¹H NMR (400 MHz, d₆-DMSO): δ ppm 12.20 (brs, 1H), 4.19 (q, J=7.2 Hz, 2H), 2.33 (s, 3H), 2.22 (s, 3H), 1.26 (t, J=7.2 Hz, 3H); LCMS-08 (Method-A): Rt 1.05 min, m/z 169.0 [M+H]⁺.

Step 3: To a stirred solution of ethyl 2,4-dimethyl-1H-imidazole-5-carboxylate (2.9 g, 17 mmol, 1.0 equiv) and benzyl(2-hydroxyethyl)carbamate (CAS: 77987-49-6) (5.04 g, 25.9 mmol, 1.5 equiv) in THF (29 mL) at −40° C. under nitrogen atmosphere. The triphenylphosphine (16.86 g, 51.72 mmol, 3.0 equiv.) was added portion-wise, followed by the addition of DIAD (10.45 g, 51.72 mmol, 3.0 equiv.) dropwise and the reaction mixture was stirred at room temperature for 14 h. After completion of the reaction, as indicated by TLC (70% EtOAc in hexane as mobile phase) the reaction mixture was poured into water (50 mL) and extracted with EtOAc (3× 20 mL). The organic layers were dried over Na₂SO₄ and concentrated under reduced pressure to afford the crude. The crude compound was purified by normal phase column chromatography using (230-400 silica gel, 0-60% EtOAc in hexane) to afford ethyl 1-(2-(((benzyloxy)carbonyl)amino)ethyl)-2,4-dimethyl-1H-imidazole-5-carboxylate as a white solid (1.5 g, 25% yield). LCMS-02 (Method-A): Rt 1.70 min, m/z 346.1 [M+H]⁺.

Step 4: To a stirred solution of ethyl 1-(2-(((benzyloxy)carbonyl)amino)ethyl)-2,4-dimethyl-1H-imidazole-5-carboxylate (1.4 g, 4.1 mmol, 1.0 equiv) in MeOH (14 mL), was added 10% Pd/C (0.28 g, 20% w/w) at room temperature. The flask was filled with N₂ first and replaced with a hydrogen atmosphere using an H₂ balloon. The reaction mixture was vigorously stirred at room temperature for 24 h. After completion of the reaction, as indicated by TLC (10% MeOH in DCM as a mobile phase), the reaction mixture was filtered through a celite bed and the bed was washed with MeOH (50 mL). The filtrate was collected and concentrated under reduced pressure to afford ethyl 1-(2-aminoethyl)-2,4-dimethyl-1H-imidazole-5-carboxylate as a white solid (0.5 g, 58% yield); ¹H NMR (400 MHz, d₆-DMSO): δ ppm 4.23 (q, J=7.2 Hz, 2H), 4.14 (t, J=6.4 Hz, 2H), 3.17 (s, 2H), 2.80 (t, J=6.4 Hz, 2H), 2.33 (s, 3H), 2.30 (s, 3H), 1.29 (t, J=7.2 Hz, 3H); LCMS-03 (Method-B): Rt 1.11 min, m/z 212.18 [M+H]⁺.

Step 5: To a stirred solution of ethyl 1-(2-aminoethyl)-2,4-dimethyl-1H-imidazole-5-carboxylate (0.48 g, 2.3 mmol, 1.0 equiv.) in toluene (4.8 mL), was added TMA (1.0 M solution in toluene) (4.2 mL, 4.5 mmol, 2.0 equiv.) at room temperature and the reaction mixture was heated at 80° C. for 2 h. After completion of the reaction as indicated by TLC (10% MeOH in DCM as a mobile phase), the reaction mixture was diluted with a MeOH (20 mL) and filtered through a celite bed. The filtrate was concentrated under vacuum to get the crude material. The obtained crude material was purified by normal phase column chromatography (230-400 silica, gradient 0-7% MeOH in DCM) to afford 1,3-dimethyl-6,7-dihydroimidazo[1,5-a]pyrazin-8(5H)-one as a yellow solid (0.2 g, 53% yield). ¹H NMR (400 MHz, d₆-DMSO): δ ppm 7.62 (brs, 1H), 3.96 (t, J=5.6 Hz, 2H), 3.45-3.41 (m, 2H), 2.28 (m, 3H), 2.26 (m, 3H); LCMS-08 (Method-B): Rt 0.48 min, m/z 166.08 [M+H]⁺.

### Intermediate 10: Synthesis of 4-bromo-2-(3-fluoro-5-(trifluoromethyl)benzyl)pyridine

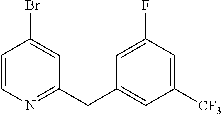 [SMILES: Fc1cc(Cc2cc(Br)ccn2)cc(C(F)(F)F)c1]

### General Method 2:

Step 1: Preparation of aryl magnesium bromide solution: To an oven-dried 3 neck round-bottom flask was added magnesium turnings (65 mg, 2.67 mmol) and anhydrous THF (2 mL). A small piece of iodine was added to the solution as an initiator and the solution was heated to 65° C. under N₂ atmosphere. After 15 min, a solution of 1-bromo-3-fluoro-5-(trifluoromethyl)benzene (500 mg, 2.06 mmol) in THF (0.5 mL) was added dropwise. The reaction was kept at 65° C. until most of Mg disappeared, and then cooled to RT for use.

To a solution of 4-bromo-2-pyridinecarboxaldehyde (230 mg, 1.23 mmol) in THF (5 mL) was added the freshly prepared BrMgAr solution at 0° C. under N₂. The solution was stirred at room temperature until the starting materials were consumed (2 h). The reaction was quenched by ammonium chloride solution and the mixture was extracted by EtOAc (×3). The combined organic layer was washed with brine, dried by Na₂SO₄ and concentrated in vacuo. The residue was purified by flash silica gel column chromatography eluting with EtOAc: petroleum ether (1:4) to afford (4-bromopyridin-2-yl)(3-fluoro-5-(trifluoromethyl)phenyl) methanol as a colorless oil (329 mg, 76% yield). LC/MS (APCI): m/z=350.1 [M+H]⁺, Rt 3.58 min. ¹H NMR (400 MHz, CDCl₃) δ 8.30 (d, J=5.2 Hz, 1H), 7.41 (s, 1H), 7.36-7.32 (m, 2H), 7.20 (m, 2H), 5.69 (s, 1H), 4.92 (br s, 1H).

Step 2: To a solution of (4-bromopyridin-2-yl) (3-fluoro-5-(trifluoromethyl)phenyl) methanol (200 mg, 0.57 mmol) in THF (4 mL) was sequentially added imidazole (97 mg, 1.43 mmol), PPh₃ (300 mg, 1.14 mmol), and iodine (305 mg, 1.20 mmol). The solution was heated to 60° C. for 12 h. Upon completion, the reaction mixture was quenched by adding water, and extracted with EtOAc (×3). The combined organic phase was washed with brine, dried over Na₂SO₄ and concentrated in vacuo. The residue was purified by flash silica gel column chromatography eluting with EtOAc: petroleum ether (1:5) to afford the title compound as a yellow oil (121 mg, 63% yield). LC/MS (APCI): m/z=334.0 [M+H]⁺, Rt 3.74 min. ¹H NMR (400 MHz, CDCl₃) δ 8.38 (d, J=5.3 Hz, 1H), 7.39-7.30 (m, 3H), 7.23-7.17 (m, 2H), 4.15 (s, 2H).

The following intermediates were synthesised using general method 2, using the specific reagent amounts detailed below.

### Intermediate 11: Synthesis of 2-bromo-4-(3-fluoro-5-(trifluoromethyl)benzyl)pyridine

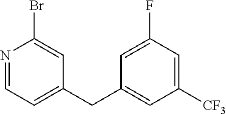 [SMILES: Fc1cc(Cc2ccnc(Br)c2)cc(C(F)(F)F)c1]

### General Method 2

Step 1: Mg (390 mg, 16.05 mmol, 1.3 equiv) in THF (5 mL), 1-bromo-3-fluoro-5-(trifluoromethyl)benzene (3.0 g, 12.35 mmol, 1 equiv) in THF (5 mL), 2-bromo-4-pyridinecarboxaldehyde (1.15 g, 6.17 mmol, 0.5 equiv) in THF (30 mL). Purification by flash column chromatography eluting with EtOAc: petroleum ether (1:4) afforded (2-bromopyridin-4-yl) (3-fluoro-5-(trifluoromethyl)phenyl) methanol as a colorless solid (1.25 g, 58% yield). LC/MS (APCI): m/z=350.0 [M+H]⁺, Rt 3.47 min. ¹H NMR (400 MHz, CDCl₃) δ 8.28 (d, J=5.0 Hz, 1H), 7.55 (s, 1H), 7.44 (s, 1H), 7.29-7.23 (m, 3H), 5.82 (s, 1H).

Step 2: (2-bromopyridin-4-yl) (3-fluoro-5-(trifluoromethyl)phenyl) methanol (1.00 g, 2.86 mmol, 1 equiv), imidazole (2.5 equiv), PPh₃ (2 equiv), iodine (2 equiv), THF (15 mL). Purification by flash column chromatography eluting with EtOAc: petroleum ether (1:5) affords the title compound as a colorless oil (425 mg, 45% yield). LC/MS (APCI): m/z=334.1 [M+H]⁺, Rt 3.64 min. ¹H NMR (400 MHz, CDCl₃) δ 8.29 (d, J=5.1 Hz, 1H), 7.30 (s, 1H), 7.23 (d, J=8.1 Hz, 2H), 7.10-6.99 (m, 2H), 3.99 (s, 2H).

### Intermediate 12: Synthesis of 2-bromo-4-(3-chloro-4,5-difluorobenzyl)pyridine

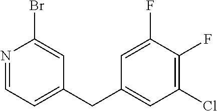 [SMILES: Fc1cc(Cc2ccnc(Br)c2)cc(Cl)c1F]

### General Method 2

Step 1: Mg (278 mg, 11.4 mmol, 1.3 equiv) in THF (5 mL), 5-bromo-1-chloro-2,3-difluorobenzene (2.0 g, 8.79 mmol, 1 equiv) in THF (5 mL), 2-bromo-4-pyridinecarboxaldehyde (817 mg, 4.39 mmol, 0.5 equiv) in THF (5 mL). Purification by flash column chromatography eluting with EtOAc: petroleum ether (1:4) afforded (2-bromopyridin-4-yl) (3-chloro-4,5-difluorophenyl) methanol as a pale-orange solid (952 mg, 65%). LC/MS: m/z=334.0 [M+H]⁺, Rt 3.48 min.

Step 2: (2-bromopyridin-4-yl) (3-chloro-4,5-difluorophenyl) methanol (1.30 g, 3.89 mmol, 1 equiv), imidazole (2.0 equiv), PPh₃ (1.1 equiv), iodine (1.2 equiv), THF (10 mL). Purification by flash column chromatography eluting with EtOAc: petroleum ether (1:5) affords the title compound as colorless solid (418 mg, 34% yield). LC/MS: m/z=316.0 [M+H]⁺, Rt 3.54 min. ¹H NMR (400 MHz, CDCl₃) δ 8.30 (d, J=5.1 Hz, 1H), 7.28 (s, 1H), 7.03 (dd, J=5.1, 1.5 Hz, 1H), 7.00-6.97 (m, 1H), 6.91-6.86 (m, 1H), 3.87 (s, 2H).

### Intermediate 13: Synthesis of 2-bromo-4-(3-(difluoromethyl)-5-fluorobenzyl)pyridine

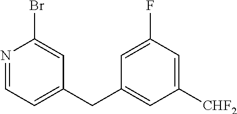 [SMILES: Fc1cc(Cc2ccnc(Br)c2)cc(C(F)F)c1]

### General Method 2

Step 1: Mg (211 mg, 8.67 mmol, 1.3 equiv) in THF (5 mL), 1-bromo-3-fluoro-5-(trifluoromethyl)benzene (1.5 g, 6.67 mmol, 1 equiv) in THF (5 mL), 2-bromo-4-pyridinecarboxaldehyde (1.11 g, 5.99 mmol, 0.9 equiv) in THF (20 mL). Purification by flash column chromatography eluting with EtOAc: petroleum ether (1:4) afforded (2-bromopyridin-4-yl) (3-(difluoromethyl)-5-fluorophenyl) methanol as a colorless oil (1.21 g, 61% yield). LC/MS (APCI): m/z=332.0 [M+H]⁺, Rt 3.49 min. ¹H NMR (400 MHz, CDCl₃) δ 8.32 (d, J=5.1 Hz, 1H), 7.55 (d, J=1.5 Hz, 1H), 7.29 (s, 1H), 7.23 (dd, J=5.2, 1.6 Hz, 1H), 7.21-7.15 (m, 2H), 6.62 (t, J=56.1 Hz, 1H), 5.80 (s, 1H).

Step 2: (2-bromopyridin-4-yl) (3-(difluoromethyl)-5-fluorophenyl) methanol (430 mg, 1.29 mmol, 1 equiv), imidazole (2.5 equiv), PPh₃ (2.0 equiv), iodine (2.0 equiv), THF (8 mL). Purification by flash column chromatography eluting with EtOAc: petroleum ether (1:4) affords the title compound as pale-yellow oil (418 mg, 34% yield). LC/MS (APCI): m/z=315.9 [M+H]⁺, Rt 3.98 min. ¹H NMR (400 MHz, CDCl₃) δ 8.26 (d, J=5.1 Hz, 1H), 7.28 (s, 1H), 7.12-7.10 (m, 2H), 7.04 (d, J=5.1 Hz, 1H), 6.96 (d, J=9.1 Hz, 1H), 6.59 (t, J=56.2 Hz, 1H), 3.96 (s, 2H).

### Intermediate 14: Synthesis of 3-ethyl-1-methyl-6,7-dihydroimidazo[1,5-a]pyrazin-8(5H)-one

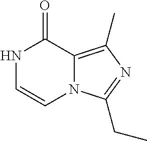

Step 1: To a solution of 1-methyl-6,7-dihydroimidazo[1,5-a]pyrazin-8(5H)-one (300 mg, 1.98 mmol) in MeCN (10 mL) was added NBS (530 mg, 2.98 mmol) at rt and the mixture was stirred in the dark at RT for 24 h. After that, the solvent was removed and the residue was purified by flash column chromatography eluting with EtOAc: MeOH (9:1) to afforded 3-bromo-1-methyl-6,7-dihydroimidazo[1,5-a]pyrazin-8(5H)-one as a yellow solid (284 mg, 62% yield). LC/MS (APCI): m/z=229.9 [M+H]⁺, Rt 2.32 min. ¹H NMR (400 MHz, DMSO-d6) δ 7.87 (s, 1H), 4.03-3.97 (m, 2H), 3.51-3.46 (m, 2H), 2.33 (s, 3H).

Step 2: Cs₂CO₃ (425 mg, 1.30 mmol) was added to a stirred solution of 3-bromo-1-methyl-6,7-dihydroimidazo[1,5-a]pyrazin-8(5H)-one (100 mg, 0.44 mmol) and vinylboronic pinacol ester (96 μL, 0.57 mmol) in 1,4-dioxane (4.5 mL) and water (0.5 mL). The reaction was degassed with N₂ for 20 min then Pd(dppf)₂Cl₂·CH₂Cl₂ complex (36 mg, 0.044 mmol) was added. The reaction mixture was heated at 100° C. for 16 h. The reaction mixture was partitioned between water (20 mL) and EtOAc (50 mL). The organic layer was separated, and the water layer was extracted with EtOAc (2×50 mL). The combined organic layer was dried over MgSO₄ and removed in vacuo to afford crude 1-methyl-3-vinyl-6,7-dihydroimidazo[1,5-a]pyrazin-8(5H)-one as a black sticky solid (41 mg) which was used for the next step without purification. LC/MS (APCI): m/z=178.1 [M+H]⁺, Rt 0.53 min.

Step 3: To a solution of crude 1-methyl-3-vinyl-6,7-dihydroimidazo[1,5-a]pyrazin-8(5H)-one (41 mg, 0.23 mmol) in MeOH (4 mL) was added 10% Pd/C (8 mg, 20% wt). The flask was filled with nitrogen first and replaced with hydrogen atmosphere using H₂ balloon. The reaction was vigorously stirred for 24 h. After completion, the reaction was filtered through Celite® pad and washed with MeOH. The solvent was removed, and the residue was purified by column chromatography eluting with EtOAc: MeOH (5:2) to afford the title compound as a light brown solid (21 mg, 27% after 2 steps). LC/MS (APCI): m/z=180.1 [M+H]⁺, Rt 0.53 min. ¹H NMR (400 MHz, DMSO-d6) δ 7.60 (s, 1H), 4.02-3.93 (m, 2H), 3.46-3.40 (m, 2H), 2.61 (q, J=7.6 Hz, 2H), 2.29 (s, 3H), 1.18 (t, J=7.5 Hz, 3H).

### Intermediate 15: Synthesis of 4-chloro-2-(3-(difluoromethyl)-5-fluorobenzyl)-2,3-dihydro-1H-pyrrolo[3,4-c]pyridin-1-one

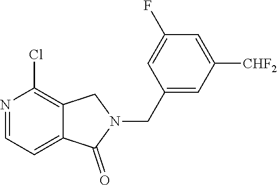 [SMILES: O=C1c2ccnc(Cl)c2CN1Cc1cc(F)cc(C(F)F)c1]

Step 1: To a solution of methyl 2-chloro-3-methylisonicotinate (200 mg, 1.08 mmol) in CF₃Ph (3 mL) was added NBS (288 mg, 1.62 mmol) and AIBN (35.40 mg, 0.22 mmol) at RT, and the mixture was stirred at 80° C. for 1 h. The mixture was poured into water and extracted with EtOAc (×3). The combined organic layer was washed with brine, dried over MgSO₄, and concentrated in vacuo. The residue was purified by flash silica gel column chromatography eluting with EtOAc: petroleum ether (1:3) to give methyl 3-(bromomethyl)-2-chloroisonicotinate as a light brown oil (241 mg, 85% yield). LC/MS (APCI): m/z=263.9 [M+H]⁺, Rt 3.619 min. ¹H NMR (400 MHz, CDCl₃) δ 8.45 (d, J=5.0 Hz, 1H), 7.68 (d, J=5.0 Hz, 1H), 5.03 (s, 2H), 4.00 (s, 3H).

Step 2: To a solution of methyl 3-(bromomethyl)-2-chloroisonicotinate (241 mg, 0.91 mmol) in CH₃CN (2.5 mL) at 0° C. was added ammonia (28% in H₂O, 2.5 mL, 18.22 mmol) and the mixture was stirred at RT for 16 h. Upon completion, water was added and the mixture was extracted with EtOAc (×3). The combined organic layer was washed with brine, dried over MgSO₄, and concentrated in vacuo. The residue was purified by flash silica gel column chromatography eluting with EtOAc: petroleum ether (2:1) to give 4-chloro-2,3-dihydro-1H-pyrrolo[3,4-c]pyridin-1-one as a pale yellow solid (92 mg, 60% yield). LC/MS (APCI): m/z=169.1 [M+H]⁺, Rt 2.655 min. ¹H NMR (400 MHz, CDCl₃) δ 8.60 (d, J=5.0 Hz, 1H), 7.72 (d, J=4.9 Hz, 1H), 6.98 (s, 1H), 4.52 (d, J=1.2 Hz, 2H).

Step 3: A mixture of 4-chloro-2,3-dihydro-1H-pyrrolo[3,4-c]pyridin-1-one (100 mg, 0.59 mmol), 1-(bromomethyl)-3-fluoro-5-(difluoromethyl)benzene (170 mg, 0.71 mmol) and K₂CO₃ (164 mg, 1.19 mmol) in MeCN (4 mL) was stirred at 70° C. for 16 h. Upon completion, water (10 mL) was added, and the mixture was extracted with EtOAc (3×50 mL). The combined organic layer was washed with brine, dried over MgSO₄ and concentrated in vacuo. The residue was purified by flash silica gel column chromatography eluting with EtOAc: petroleum ether (1:4) to afford the title compound as a colorless solid (92 mg, 47% yield). LC/MS (APCI): m/z=327.0 [M+H]⁺, Rt 3.249 min. ¹H NMR (400 MHz, CDCl₃) δ 8.60 (d, J=4.9 Hz, 1H), 7.74 (d, J=4.9 Hz, 1H), 7.25 (d, J=1.3 Hz, 1H), 7.20 (d, J=8.5 Hz, 1H), 7.15 (d, J=8.8 Hz, 1H), 6.62 (t, J=56.1 Hz, 1H), 4.85 (s, 2H), 4.36 (s, 2H).

### Intermediate 16: Synthesis of 2-chloro-4-(3-(difluoromethoxy)-5-fluorobenzyl)pyridine

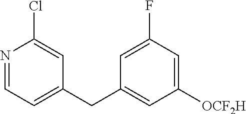

K₂CO₃ (244 mg, 1.76 mmol) was added to a stirred solution of methyl 3-fluoro-5-(difluoromethoxy)benzyl bromide (150 mg, 0.59 mmol) and 2-chloropyridine-4-boronic acid (93 mg, 0.59 mmol) in 1,4-dioxane (7 mL) and water (3 mL). The reaction was degassed with N₂ for 20 min then Pd(dppf)₂Cl₂·CH₂Cl₂ (48 mg, 0.059 mmol) complex was added. The reaction mixture was heated at 100° C. for 3 h. The reaction mixture was partitioned between water (50 mL) and EtOAc (50 mL). The organic layer was separated, and the water layer was extracted with EtOAc (2× 50 mL). The combined organic layer was dried over MgSO₄ and concentrated in vacuo. The residue was purified by flash column chromatography eluting with EtOAc:PET (1:3) to afford the title compound as colorless oil (87 mg, 51% yield). LC/MS (APCI): m/z=287.9 [M+H]⁺, Rt 3.878 min. ¹H NMR (400 MHz, DMSO-d6) δ 8.32 (d, J=5.1 Hz, 1H), 7.48-7.45 (m, 1H), 7.32 (dd, J=5.1, 1.5 Hz, 1H), 7.27 (s, 1H), 7.12-7.07 (m, 1H), 7.04-6.97 (m, 2H), 4.02 (s, 2H).

### Intermediate 17: Synthesis of 3-((2-chloropyridin-4-yl)methyl)-5-fluorobenzonitrile

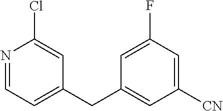 [SMILES: N#Cc1cc(F)cc(Cc2ccnc(Cl)c2)c1]

K₂CO₃ (2.52 g, 18.22 mmol) was added to a stirred solution of 3-(bromomethyl)-5-fluorobenzonitrile (1.30 g, 6.07 mmol) and 2-chloropyridine-4-boronic acid (956 mg, 6.07 mmol) in 1,4-dioxane (28 mL) and water (12 mL). The reaction was degassed with N₂ for 20 min then Pd(dppf)₂Cl₂·CH₂Cl₂ (496 mg, 0.61 mmol) complex was added. The reaction mixture was heated at 100° C. for 4 h. The reaction mixture was partitioned between water (100 mL) and EtOAc (200 mL). The organic layer was separated, and the water layer was extracted with EtOAc (2×200 mL). The combined organic layer was dried over MgSO₄ and concentrated in vacuo. The residue was purified by flash silica gel column chromatography eluting with EtOAc:PET (1:3) to afford the title compound as a colorless solid (756 mg, 50% yield). LC/MS (APCI): m/z=247.0 [M+H]⁺, Rt 3.69 min, ¹H NMR (400 MHz, CDCl₃) δ 8.34 (d, J=5.1 Hz, 1H), 7.30-7.27 (m, 2H), 7.14-7.11 (m, 2H), 7.01 (dd, J=5.1, 1.6 Hz, 1H), 3.99 (s, 2H).

### Intermediate 18: Synthesis of 2-bromo-4-(3-(difluoromethyl)-5-fluorobenzyl)pyridine

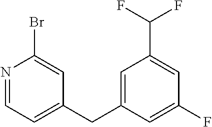 [SMILES: Fc1cc(Cc2ccnc(Br)c2)cc(C(F)F)c1]

To a stirred solution of 1-(chloromethyl)-3-(difluoromethyl)-5-fluorobenzene (6.0 g, 30.835 mmol, 1.0 equiv) in DMF (60.0 mL), was added (2-bromopyridin-4-yl) boronic acid (CAS: 458532-94-0) (9.33 g, 46.6 mmol, 1.5 equiv), K₃PO₄ (13.14 g, 61.67 mmol, 2.0 equiv) at room temperature. The reaction mixture was purged with N₂ for 15 min, then the catalyst Pd(PPh₃)₄ (1.78 g, 1.54 mmol, 0.05 equiv) was added and the sealed tube was heated up to 70° C. for 5 h. After completion of the reaction as indicated by TLC (20% EtOAc in hexane), the reaction mixture was diluted with ice-cold water (700 mL) and extracted with EtOAc (3×500 mL). The organic layers were dried over Na₂SO₄ and concentrated under reduced pressure to afford the crude compound. The crude was purified by normal phase column chromatography (230-400 silica, gradient 20% EtOAc in hexane) to afford the title compound as light-yellow solid (2.05 g, 21% yield); ¹H NMR (400 MHz, d₆-DMSO): δ ppm 8.30 (d, J=5.2 Hz, 1H), 7.63 (s, 1H), 7.42-7.30 (m, 4H), 7.02 (t, J=55.6, 1H), 4.07 (s, 2H). LCMS-02 (Method-A): Rt 2.49 min, m/z 318.2 [M+H]⁺.

Intermediate 19: Synthesis of 4-chloro-6-(3-(difluoromethyl)-5-fluorobenzyl)pyrimidine

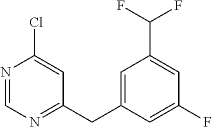 [SMILES: Fc1cc(Cc2cc(Cl)ncn2)cc(C(F)F)c1]

To a stirred solution of 1-(chloromethyl)-3-(difluoromethyl)-5-fluorobenzene (2.0 g, 10.3 mmol, 2.0 equiv) in THF (10 mL) was added Zn metal (1.34 g, 20.6 mmol, 2.0 equiv) at room temperature and the reaction mixture was stirred at 80° C. for 30 min. The reaction mixture was allowed to cool to room temperature. After cooling, the 4,6-dichloropyrimidine (CAS: 1193-21-1) (3.0 g, 20.61 mmol, 2.0 equiv) and PdCl₂(PPh₃)₂ (0.72 g, 1.03 mmol, 0.1 equiv) were added at room temperature, then the reaction mixture was stirred again at 80° C. for 16 h. After completion of the reaction as indicated by TLC (30% EtOAc in hexane), the reaction mixture was filtered through a celite bed and washed with EtOAc (2×20 mL). The filtrate was diluted with water (25 mL) and extracted with EtOAc (2×25 mL). The organic layers were dried over Na₂SO₄ and concentrated under reduced pressure to get the crude. The crude was purified by normal phase column chromatography using (230-400 silica, gradient 0-15% EtOAc in hexane) to afford the title compound as a yellow oil (0.22 g, 8% yield); ¹H NMR (400 MHz, d₆-DMSO): δ ppm 8.97 (d, J=0.8 Hz, 1H), 7.76 (d, J=0.8 Hz, 1H), 7.41-7.39 (m, 2H), 7.31 (d, J=1.2 Hz, 1H), 7.03 (t, J=55.6 Hz, 1H), 4.21 (s, 2H); LCMS-08 (Method-A): Rt 2.18 min, m/z 272.98 [M+H]⁺.

### Intermediate 20: Synthesis of 4-bromo-2-(3-(difluoromethyl)-5-fluorobenzyl)pyridine

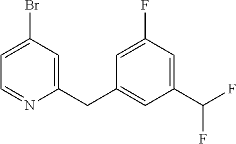 [SMILES: Fc1cc(Cc2cc(Br)ccn2)cc(C(F)F)c1]

Step 1: To a stirred solution of (3-(difluoromethyl)-5-fluorophenyl) methanol (2.0 g, 11.35 mmol, 1.0 equiv) in DCM (20.0 mL) was added MnO₂ (9.87 g, 114 mmol, 10.0 equiv) and stirred at RT for 24 h. After completion of the reaction as indicated by TLC (20% EtOAc in hexane), the reaction mixture was filtered through a Celite bed and the bed was washed with DCM (60 mL). The filtrate was concentrated under reduced pressure to afford 3-(difluoromethyl)-5-fluorobenzaldehyde as a yellowish oil (1.5 g, 75% yield). ¹H NMR (400 MHz, d₆-DMSO): δ ppm 10.05 (s, 1H), 8.00 (s, 1H), 7.90 (d, J=8.4 Hz, 1H), 7.82 (d, J=8.8 Hz, 1H), 7.17 (t, J=55.6 Hz, 1H).

Step 2: To a stirred solution of 2,4-dibromo pyridine (CAS: 58530-53-3) (1.20 g, 5.06 mmol, 1.0 equiv) in toluene (12.0 mL) at −78° C., was added n-BuLi (2.6 M in hexane) (3.89 mL, 10.1 mmol, 2.0 equiv) and the reaction stirred at −78° C. for 2 h. To this reaction mixture was added 3-(difluoromethyl)-5-fluorobenzaldehyde (1.32 g, 7.60 mmol, 1.5 eq) at −78° C. The reaction mixture was stirred at the same temperature for 1 h, then allowed to warm to 0° C. and stirred for another 4 h. After completion of the reaction as indicated by TLC (20% EtOAc in hexane), the reaction mixture was quenched with 1N HCl (pH=4-5) and extracted with EtOAc (3×50 mL). The organic layers were dried over Na₂SO₄ and concentrated under reduced pressure to obtain the crude. The crude was purified by normal phase column chromatography using (230-400 silica gel, 10% EtOAc in hexane) to afford the two separated regio-isomers. NOE analysis determined the first eluted regio-isomer (white solid, 0.90 g, 53% yield) as the desired regio-isomer (4-bromopyridin-2-yl) (3-(difluoromethyl)-5-fluorophenyl) methanol ¹H NMR (400 MHz, d₆-DMSO): δ ppm 8.38 (d, J=5.2 Hz, 1H), 7.84 (d, J=2.0 Hz, 1H), 7.56 (dd, J=5.2, 2.0 Hz, 1H), 7.48 (s, 1H), 7.44-7.39 (m, 1H), 7.30 (d, J=8.8 Hz, 1H), 7.05 (t, J=55.6 Hz, 1H), 6.54 (d, J=4.4 Hz, 1H), 5.81 (d, J=4.4 Hz, 1H). LCMS-01 (Method-A): Rt 1.70 min, m/z 333.7 [M+H]⁺.

Step 3: To a stirred solution of (4-bromopyridin-2-yl) (3-(difluoromethyl)-5-fluorophenyl) methanol (0.9 g, 2.71 mmol, 1.0 equiv) in THF (9.0 mL) at room temperature, was added PBr₃ (1.46 g, 5.42 mmol, 2.0 equiv) and stirred the reaction mixture at 80° C. for 3 h. After completion of the reaction as indicated by TLC (20% EtOAc in hexane), the reaction mixture was quenched with 10% aq. NaHCO₃ (pH=11) and extracted with EtOAc (3×30 mL). The organic layer was dried over Na₂SO₄ and concentrated under reduced pressure to afford the crude compound. The crude compound was purified by normal phase column chromatography (230-400 silica, 0-10% EtOAc in hexane) to afford 4-bromo-2-(3-(difluoromethyl)-5-fluorobenzyl)pyridine as a white solid. (0.3 g, 35% yield). ¹H NMR (400 MHz, d₆-DMSO): δ ppm 8.39 (d, J=5.6 Hz, 1H), 7.71 (d, J=1.6 Hz, 1H), 7.53 (dd, J=5.2, 1.6 Hz, 1H), 7.37-7.35 (m, 2H), 7.28 (d, J=9.2 Hz, 1H), 7.02 (t, J=55.6 Hz, 1H), 4.17 (s, 2H). LCMS-01 (Method-A): 1.90 min, m/z 317.7 [M+H]⁺ for ⁸¹Br.

### Intermediate 21: Synthesis of 2-chloro-4-(3-(difluoromethyl)-5-fluorobenzyl)pyridine

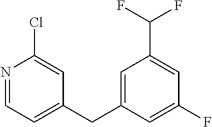 [SMILES: Fc1cc(Cc2ccnc(Cl)c2)cc(C(F)F)c1]

Step 1: Note: 4×5.0 g reactions were carried out in parallel: To each stirred solution of dimethyl 5-fluoroisophthalate (5.0 g, 23.6 mmol, 1.0 eq) in anhydrous THF (100 mL) under nitrogen atmosphere at −20° C., was added LiAlH₄ (9.4 mL, 1.0 M in THF) dropwise. After the additions were completed, the reaction mixtures were stirred for 1 h at the same temperature. After completion of the reactions as indicated by TLC (using 25% EtOAc in hexane) the reaction mixtures were neutralized with aqueous 2 N HCl solution (30 mL), filtered through a Celite bed and the bed washed with EtOAc (100 mL). The filtrates were collected and washed with water (50 mL). The organic layers were dried over sodium sulphate, filtered and concentrated under vacuum to get the crude products. The crudes were purified by normal phase glass column chromatography using (60-120 silica gel, 0-20% EtOAc in hexane) to afford methyl 3-fluoro-5-(hydroxymethyl)benzoate as a clear oil (8.1 g, 47% combined yield across 4 parallel batches). ¹H NMR (DMSO-d6, 400 MHz): δ ppm 7.79 (s, 1H), 7.54 (d, J=8.8 Hz, 1H), 7.43 (d, J=9.2 Hz, 1H), 5.49 (t, J=5.6 Hz, 1H), 4.57 (d, J=5.6 Hz, 2H), 3.87 (s, 3H).

Step 2: A stirred solution of methyl 3-fluoro-5-(hydroxymethyl)benzoate (4.05 g, 0.021 mmol, 1.0 eq) in DCM (122 mL) under N₂ (g) atmosphere was cooled to 0° C. then Dess-Martin periodinane was added portion wise and the reaction mixture stirred, warming from 0° C. to room temperature for 2 h. After completion of the reaction as indicated by TLC (using 20% EtOAc in hexane) the reaction mixture was filtered and washed with DCM (100 mL). The filtrate was collected and concentrated under reduced pressure to get the crude. The crude was purified by normal phase flash column chromatography using (230-400 mesh silica gel), 0-7% EtOAc in hexane eluents to afford methyl 3-fluoro-5-formylbenzoate as an off white solid (6.4 g, 80% yield). ¹H NMR (DMSO-d6, 400 MHz): δ ppm 10.08 (s, 1H), 8.33 (s, 1H), 8.02 (t, J=8.4 Hz, 1H), 3.92 (s, 3H).

Step 3: A stirred solution of methyl 3-fluoro-5-formylbenzoate (6.4 g, 35.1 mmol, 1.0 eq) in DCM (128 mL) under N₂ (g) atmosphere was cooled to 0° C. and DAST (11.32 g, 70.27 mmol, 2.0 eq) was added slowly. The resulting reaction mixture was stirred at 10° C. for 3 h. After completion of the reaction as indicated by TLC (using 20% EtOAc in hexane) the reaction mixture was neutralized with saturated sodium bicarbonate solution (200 mL) and extracted with DCM (3× 40 mL). The combined organic layer was dried over sodium sulphate, filtered and concentrated under vacuum to give the crude product. The crude was purified on normal phase flash column chromatography using (230-400 mesh silica gel) 0-7% EtOAc in hexane eluents to afford methyl 3-(difluoro methyl)-5-fluorobenzoate as a clear oil (3.9 g, 54% yield) ¹H NMR (DMSO-d6, 400 MHz): δ ppm 7.98 (s, 1H), 7.88 (dd, J=0.8 Hz, 8.0 Hz, 1H), 7.78 (d, J=8.0 Hz, 1H), 7.16 (t, J=55.2 Hz, 1H), 3.90 (s, 3H).

Step 4: A stirred solution of methyl 3-(difluoromethyl)-5-fluorobenzoate (3.9 g, 19.1 mmol, 1.0 eq) in THF (39.0 mL) under N₂ (g) atmosphere was cooled to 0° C. The LiAlH₄ solution 1.0 M in THF (9.5 mL, 9.55 mmol, 0.5 eq) was added dropwise and the resulting reaction mixture stirred at room temperature for 2 h. After completion of the reaction as indicated by TLC (30% EtOAc in hexane) the reaction mixture was quenched with 2N HCl solution (40 mL) and extracted with EtOAc (3× 30 mL). The combined organic layer was dried over sodium sulphate and concentrated under vacuum to get crude (3-(difluoromethyl)-5-fluorophenyl) methanol as a clear oil (3.9 g, quantitative yield). The crude material was used in the next step without any further purification. ¹H NMR (DMSO-d6, 400 MHz): δ ppm 7.39 (br s, 1H), 7.29 (t, J=9.6 Hz, 2H), 7.05 (t, J=56 Hz, 1H), 5.47 (t, J=5.6 Hz, 1H), 4.56 (d, J=5.6 Hz, 2H).

Step 5: To a stirred solution of (3-(difluoromethyl)-5-fluorophenyl) methanol (3.4 g, 19.3 mmol, 1.0 eq) in chloroform (68 mL) was added thionyl chloride (34 mL) and the resulting solution heated to 65° C. for 4 h. After completion of the reaction as indicated by TLC (using 20% EtOAc in hexane) the reaction mixture was quenched with saturated sodium bicarbonate solution (560 mL) and extracted with EtOAc (3× 50 mL). The combined organic layer was dried over sodium sulphate, filtered and concentrated under vacuum to get 1-(chloromethyl)-3-(difluoromethyl)-5-fluorobenzene as a crude brown oil (2.85 g, 76% yield). The crude material was used for next step without further purification. ¹H NMR (DMSO-d6, 400 MHz): δ ppm 7.53-7.50 (m, 2H), 7.43 (d, J=8.4 Hz, 1H), 7.07 (t, J=55.2 Hz, 1H), 4.84 (s, 2H).

Step 6: A stirred solution of (2-chloropyridin-4-yl) boronic acid (CAS: 458532-96-2) (1.2 g, 6.16 mmol, 1.0 eq) and 1-(chloromethyl)-3-(difluoromethyl)-5-fluorobenzene (1.2 g, 6.77 mmol, 1.1 eq) in 1,4-dioxane:water (24 mL, 8:2) was purged with N₂ (g) for 10 min and to this mixture KOAc (1.81 g, 18.48 mmol, 3.0 eq) and PdCl₂ (dppf) (0.25 g, 0.30 mmol, 0.05 eq) were added and reaction mixture heated to 90° C. for 2 h. After completion of the reaction as indicated by TLC (using 15% EtOAc in hexane) the reaction mixture was filtered through a Celite bed and the bed washed with EtOAc (50 mL). The filtrate was washed with water (10 mL). The organic layer was dried over sodium sulphate, filtered and concentrated under vacuum to get the crude product. The crude was purified by normal phase column chromatography (using 230-400 mesh silica gel), 0-10% EtOAc in hexane eluent to afford 2-chloro-4-(3-(difluoromethyl)-5-fluorobenzyl)pyridine as a clear oil (0.8 g, 48% yield). ¹H NMR (DMSO-d6, 400 MHz): δ ppm 8.32 (d, J=4.8 Hz, 1H), 7.49 (s, 1H), 7.42-7.38 (m, 2H), 7.34-7.30 (m, 2H), 7.02 (t, J=55.6 Hz, 1H), 4.08 (s, 2H) LCMS-01 (Method-A): Rt 1.82 min, m/z 271.54 [M+H]⁺.

### Intermediate 22: Synthesis of 4-chloro-2-(3-fluoro-5-(trifluoromethyl)benzyl)-2,3-dihydro-1H-pyrrolo[3,4-c]pyridine

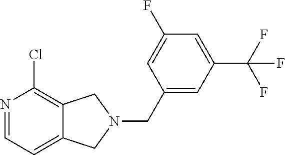 [SMILES: Fc1cc(CN2Cc3ccnc(Cl)c3C2)cc(C(F)(F)F)c1]

Step 1: To a stirred solution of pyridine-3,4-dicarboxylic acid (CAS: 490-11-9) (20.0 g, 120 mmol, 1.0 equiv) in MeOH (200 mL), was added conc. H₂SO₄ (23.45 g, 239.3 mmol, 2.0 equiv) dropwise at 0° C. and stirred the reaction mixture at 60° C. for 16 h. After completion of the reaction as indicated by TLC (70% EtOAc in hexane) the reaction mixture was quenched with saturated NaHCO₃ solution (PH ~7) and extracted with EtOAc (3×100 mL). The organic layer was dried over Na₂SO₄ and concentrated under reduced pressure to afford the crude compound. The crude was purified by trituration with pentane to afford dimethyl pyridine-3,4-dicarboxylate as a yellowish liquid (12.0 g, 51% yield). ¹H NMR (400 MHz, d₆-DMSO): δ ppm 9.02 (d, J=0.8 Hz, 1H), 8.93 (d, J=5.2 Hz, 1H), 7.71 (dd, J=5.2, 0.8 Hz, 1H), 3.87 (s, 6H).; LCMS-07 (Method A): Rt 1.67 min, m/z 196.2 [M+H]⁺.

Step 2: To a stirred solution of dimethyl pyridine-3,4-dicarboxylate (12.0 g, 61.5 mmol, 1.0 equiv) in DCM (120 mL), was added m-CPBA (~75%) (28.2 g, 123 mmol, 2.0 equiv) portion wise at 0° C. and stirred the reaction mixture at RT for 4 h. After completion of the reaction as indicated by TLC (100% EtOAc), the reaction mixture was quenched with saturated NaHCO₃ solution (pH ~7) and extracted with DCM (3× 100 mL). The organic layers were dried over Na₂SO₄ and concentrated under reduced pressure to get crude compound. The crude was purified by normal phase column chromatography (230-400 silica, gradient 80% EtOAc in hexane) to afford 3,4-bis(methoxycarbonyl)pyridine 1-oxide as a pink solid (10.0 g, 77% yield). ¹H NMR (400 MHz, d₆-DMSO): δ ppm 8.57 (d, J=0.4 Hz, 1H), 8.42 (dd, J=6.8, 2.0 Hz, 1H), 7.82 (d, J=6.8, 0.4 Hz, 1H), 3.87 (s, 3H), 3.85 (s, 3H).; LCMS-01 (Method A): Rt 1.43 min, m/z 212.1 [M+H]⁺.

Step 3: To the compound 3,4-bis(methoxycarbonyl)pyridine 1-oxide (10.0 g, 47.35 mmol, 1.0 equiv) in a round bottom flask, was added POCl₃ (100 mL) dropwise at 0° C. and stirred the reaction mixture at 60° C. for 16 h. After completion of the reaction as indicated by TLC (50% EtOAc in hexane), the reaction mixture was concentrated under reduced pressure and then quenched with saturated aq. NaHCO₃ solution (pH ~7) and extracted with EtOAc (3× 60 mL). The organic layers were dried over Na₂SO₄ and concentrated under reduced pressure to get crude compound. The crude was purified by normal phase column chromatography (230-400 silica, gradient 25% EtOAc in hexane) to afford dimethyl 2-chloropyridine-3,4-dicarboxylate, as a yellow solid (2.2 g, 20% yield). ¹H NMR (400 MHz, d₆-DMSO): δ ppm 8.74 (d, J=5.2 Hz, 1H), 7.93 (d, J=5.2 Hz, 1H), 3.91 (s, 3H); 3.90 (s, 3H); LCMS-08 (Method A): Rt 1.84 min, m/z 230.03 [M+H]⁺.

Step 4: To a stirred solution of dimethyl 2-chloropyridine-3,4-dicarboxylate (2.2 g, 9.6 mmol, 1.0 equiv) in DCM: MeOH (22 mL, 1:1), was added NaBH₄ (2.18 g, 57.6 mmol, 6.0 equiv) portion wise at 0° C. and stirred the reaction mixture at RT for 6 h. After completion of the reaction as indicated by TLC (10% MeOH in DCM), the reaction mixture was treated with formic acid and adjusted the pH of crude slightly acidic (pH~5), filtered through a celite bed and concentrated under reduced pressure then this crude was treated with 2M HCl solution (70 mL) and neutralized with saturated NaHCO₃ solution (50 mL) (pH ~7), then extracted with EtOAc (3×50 mL). The organic layers were dried over Na₂SO₄ and concentrated under reduced pressure to get the crude compound. The crude was purified by trituration with ether: pentane to afford (2-chloropyridine-3,4-diyl)dimethanol as an off white solid (1.3 g, 78% yield). ¹H NMR (400 MHz, d₆-DMSO): δ ppm 8.31 (d, J=4.8 Hz, 1H), 7.52 (d, J=4.8 Hz, 1H), 5.53 (t, J=5.6 Hz, 1H), 5.17 (t, J=5.2 Hz, 1H), 4.73 (dd, J=5.6, 0.4 Hz, 2H), 4.59 (d, J=1.6 Hz, 2H); LCMS-08 (Method A): Rt 0.72 min, m/z 174.13 [M+H]⁺.

Step 5: To a stirred solution of (2-chloropyridine-3,4-diyl)dimethanol (1.3 g, 7.5 mmol, 1.0 equiv) in DCM (26 mL) was added SOCl₂ (26 mL) dropwise at 0° C. and stirred the reaction mixture at 60° C. for 8 h. After completion of the reaction as indicated by TLC (50% EtOAc in hexane), the reaction mixture was quenched with saturated NaHCO₃ solution (pH~8) and extracted with DCM (3×30 mL). The organic layers were dried over Na₂SO₄ and concentrated under reduced pressure to get the crude compound. The crude was purified by normal phase column chromatography (230-400 silica, gradient 15% EtOAc in hexane) to afford 2-chloro-3,4-bis(chloromethyl)pyridine as a yellow solid (0.25 g, 16% yield). ¹H NMR (400 MHz, d₆-DMSO): δ ppm 8.45 (d, J=5.2 Hz, 1H), 7.60 (d, J=5.2 Hz, 1H), 4.97 (s, 2H), 4.95 (s, 2H). LCMS-01 (Method A): Rt 1.98 min, m/z 209.99 [M+H]⁺.

Step 6: To a stirred solution of 2-chloro-3,4-bis(chloromethyl)pyridine (0.25 g, 1.2 mmol, 1.0 equiv.) in DMF (2.5 mL), was added (3-fluoro-5-(trifluoromethyl)phenyl) methanamine (CAS: 150510-77-4) (0.34 g, 1.78 mmol, 1.5 equiv.) followed by dropwise addition of DIPEA (0.46 g, 3.6 mmol, 3.0 equiv.). The reaction mixture was stirred at 100° C. for 6 h. After completion of the reaction as indicated by TLC (30% EtOAc in hexane), the reaction mixture was diluted with ice-cold water (15.0 mL) and extracted with EtOAc (3×10 mL). The organic layers were dried over Na₂SO₄ and concentrated under reduced pressure to get the crude compound. The crude was purified by normal phase column chromatography (230-400 silica, gradient 12% EtOAc in hexane) to afford 4-chloro-2-(3-fluoro-5-(trifluoromethyl)benzyl)-2,3-dihydro-1H-pyrrolo[3,4-c]pyridine as yellow solid (0.21 g, 53% yield). ¹H NMR (400 MHz, d₆-DMSO): δ ppm 8.27 (d, J=5.2 Hz, 1H), 7.61-7.57 (m, 3H), 7.36 (d, J=4.8 Hz, 1H), 4.02 (m, 4H), 3.98 (s, 2H). LCMS-01 (Method A): Rt 2.22 min, m/z 331.18 [M+H]⁺.

### Intermediate 23: Synthesis of 1-(difluoro methyl)-6,7-dihydroimidazo[1,5-a]pyrazin-8(5H)-one

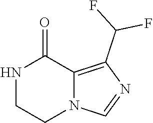 [SMILES: O=C1NCCn2cnc(C(F)F)c21]

Step 1: To a stirred solution of dimethyl 1H-imidazole-4,5-dicarboxylate (CAS: 3304-70-9) (4.0 g, 22 mmol, 1.0 eq) and benzyl(2-hydroxyethyl)carbamate (CAS: 77987-49-6) (6.3 g, 33 mmol, 1.5 eq) in THF (80.0 mL) at −40° C. under nitrogen atmosphere. The triphenyl phosphine (11.3 g, 43.4 mmol, 2.0 eq) was added portion wise, followed by addition of DIAD (8.7 g, 43.4 mmol, 2.0 eq) dropwise and the mixture was stirred at room temperature for 16 h. After completion, as indicated by TLC (100% EtOAc as mobile phase) the reaction mixture was poured into ice-cold water (500 mL) and extracted with EtOAc (2×600 mL). The organic layer was dried over Na₂SO₄ and concentrated under reduced pressure to get the crude. The crude was purified by normal phase column chromatography using (100-200 silica gel, 0-80% EtOAc in hexane) to afford dimethyl 1-(2-(((benzyloxy)carbonyl) amino)ethyl)-1H-imidazole-4,5-dicarboxylate as a white semi-solid (8.0 g, 51% yield). ¹H NMR (DMSO-d6, 400 MHz): δ ppm 7.85 (s, 1H), 7.40-7.29 (m, 6H), 4.98 (s, 2H), 4.25 (t, J=5.6 Hz, 2H), 3.80 (s, 3H), 3.78 (s, 3H), 3.31 (2H obscured by solvent peak); LCMS-01 (Method-A): Rt 1.35 min, m/z 361.95 [M+H]⁺.

Step 2: To a stirred solution of dimethyl 1-(2-(((benzyloxy)carbonyl)amino)ethyl)-1H-imidazole-4,5-dicarboxylate (8.0 g, 22 mmol, 1.0 eq) in methanol (80 mL) at room temperature was added Pd/C (4.0 g, 50% w/w) portion wise. The reaction mixture was stirred at room temperature under H₂ balloon for 16 h. The reaction was monitored by TLC (5% methanol in DCM as mobile phase) which confirmed reaction completion after 16 h. The reaction mixture was filtered through a Celite® pad (washed with methanol (1000 mL)). The filtrate was concentrated under reduced pressure to afford the crude. The crude was triturated with EtOAc to afford methyl 8-oxo-5,6,7,8-tetrahydroimidazo[1,5-a]pyrazine-1-carboxylate as a white solid (4.0 g, 93% yield); ¹H NMR (DMSO-d6, 400 MHz): δ ppm 8.17 (brs, 1H), 7.91 (s, 1H), 4.21 (t, J=5.6 Hz, 2H), 3.77 (s, 3H), 3.51-3.47 (m, 2H). LCMS-08 (Method-J): Rt 3.41 min, m/z 196.10 [M+H]⁺.

Step 3: To a stirred solution of methyl 8-oxo-5,6,7,8-tetrahydroimidazo[1,5-a] pyrazine-1-carboxylate (2.0 g, 10.24 mmol, 1.0 eq) in THF (40.0 mL) was added lithium borohydride (12.2 mL, 12.29 mmol, 1.2 eq) dropwise at 0° C. and the reaction mixture was stirred at room temperature for 2 h. The reaction completion was indicated by TLC (10% methanol in DCM as mobile phase). The resulting reaction mixture was poured into saturated NaOH solution (100 mL) and concentrated under reduced pressure to afford the crude. The crude was purified by normal phase column chromatography using (100-200 silica gel, 0-10% methanol in DCM) to afford 1-(hydroxymethyl)-6,7-dihydroimidazo[1,5-a]pyrazin-8(5H)-one as an off-white solid (1.5 g, 44% yield). ¹H NMR (DMSO-d6, 400 MHz): δ ppm 8.12 (s, 1H), 7.76 (s, 1H), 5.41 (t, J=5.6 Hz, 1H), 4.56 (d, J=5.6 Hz, 2H), 4.16 (t, J=5.2 Hz, 2H), 3.49 (brs, 2H); LCMS-01 (Method-A): Rt 0.27 min, m/z 167.88 [M+H]⁺.

Step 4: To a stirred solution of 1-(hydroxymethyl)-6,7-dihydroimidazo[1,5-a] pyrazin-8(5H)-one (0.5 g, 2.994 mmol, 1.0 eq) in DCM (10.0 mL) under nitrogen atmosphere was added Dess-Martin periodinane (1.90 g, 4.49 mmol, 1.5 eq) portion wise at 0° C. The reaction mixture was stirred at room temperature for 2 h. The completion of reaction was indicated by TLC (15% methanol in DCM as mobile phase). The resulting reaction mixture was concentrated under vacuum to afford the crude. The crude was purified by normal phase column chromatography using (100-200 silica gel, 0-17% methanol in DCM) to afford 8-oxo-5,6,7,8-tetrahydroimidazo[1,5-a]pyrazine-1-carbaldehyde as a white solid (0.2 g, 41% yield); ¹H NMR (DMSO-d6, 400 MHz): δ ppm 10.29 (s, 1H), 8.47 (brs, 1H), 7.95 (s, 1H), 4.26 (t, J=6.0 Hz, 2H), 3.56 (m, 2H); LCMS-01 (Method-A): Rt 0.27 min, m/z 165.78 [M+H]⁺.

Step 5: To a stirred solution of 8-oxo-5,6,7,8-tetrahydroimidazo[1,5-a]pyrazine-1-carbaldehyde (0.18 g, 1.09 mmol, 1.0 eq) in DCM (2 mL) under nitrogen atmosphere, was added diethylamino sulfur trifluoride (0.527 g, 3.27 mmol, 3.0 eq) dropwise at 0° C. The reaction mixture was stirred for 3 h at the same temperature. After completion of reaction as indicated by TLC (10% methanol in DCM as mobile phase), the reaction mixture was concentrated under vacuum to afford the crude. The crude material was purified by normal phase column chromatography using (100-200 silica gel, 0-17% methanol in DCM) to afford 1-(difluoro methyl)-6,7-dihydroimidazo[1,5-a]pyrazin-8(5H)-one as a white-yellowish solid (0.09 g, 44% yield). ¹H NMR (DMSO-d6, 400 MHz): δ ppm 8.28 (brs, 1H), 7.95 (s, 1H), 7.21 (t, J=54.4 Hz, 1H), 4.23 (t, J=Hz, 2H), 3.55-3.51 (m, 2H); LCMS-01 (Method-A): Rt 0.556 min, m/z 187.8 [M+H]⁺.

### Intermediate 24: Synthesis of 2-chloro-4-(3,4,5-trifluorobenzyl)pyridine

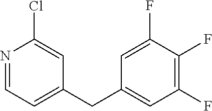 [SMILES: Fc1cc(Cc2ccnc(Cl)c2)cc(F)c1F]

To a stirred solution of (2-chloropyridin-4-yl) boronic acid (CAS: 458532-96-2) (0.500 g, 3.81 mmol, 1.0 equiv) and 5-(bromomethyl)-1,2,3-trifluorobenzene (CAS: 220141-72-0) (0.710 g, 3.81 mmol, 1.0 equiv) in 1,4-dioxane (4.0 mL): water (1.0 mL), was added Na₂CO₃ (0.647 g, 6.37 mmol, 2.0 equiv) at room temperature and the reaction mixture was degassed with N₂ for 10 min. The catalyst PdCl₂(dppf)·DCM (0.129 g, 0.159 mmol, 0.05 equiv) was added and the reaction mixture was heated at 90° C. for 3 h. After completion of the reaction as indicated by TLC (50% EtOAc in hexane as a mobile phase), the reaction mixture was diluted with water (10 mL) and extracted with EtOAc (3× 20 mL). The combined organic layers were dried over Na₂SO₄ and concentrated under reduced pressure to afford the crude compound. The crude compound was purified by normal phase column chromatography (230-400 silica, 0-30% EtOAc in hexane) to afford the title compound as white solid (0.220 g, 27% yield); ¹H NMR (400 MHz, d₆-DMSO): δ ppm 8.32 (dd, J=5.1, 0.6 Hz, 1H), 7.47 (dd, J=1.4, 0.6 Hz, 1H), 7.37-7.22 (m, 3H), 3.99 (s, 2H); LCMS-08 (Method-A): Rt 2.24 min, m/z 258.0 [M+H]⁺.

### Intermediate 25: Synthesis of 4-bromo-2-(3-fluoro-5-(trifluoromethyl)benzyl)-2H-pyrazolo[4,3-c]pyridine

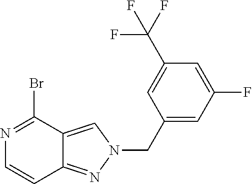 [SMILES: Fc1cc(Cn2cc3c(Br)nccc3n2)cc(C(F)(F)F)c1]

To a stirred solution of 4-bromo-2H-pyrazolo[4,3-c]pyridine (CAS: 1159829-63-6) (1.0 g, 5.0549 mmol, 1.0 equiv) in DMF (10.0 mL), was added 1-(bromomethyl)-3-fluoro-5-(trifluoromethyl)benzene (CAS: 239087-09-3) (1.94 g, 7.57 mmol, 1.5 equiv) and K₂CO₃ (2.09 g, 15.2 mmol, 3.0 equiv) at room temperature. The reaction mixture was stirred at 120° C. for 4 h. After completion of the reaction as indicated by TLC (20% EtOAc in hexane), the reaction mixture was diluted with ice-cold water (50 mL) and extracted with EtOAc (3×40 mL). The combined organic layers were dried over Na₂SO₄ and concentrated under reduced pressure to afford the crude compound. The crude was purified by normal phase column chromatography using (100-200 silica, gradient 8% EtOAc in hexane) to afford the title compounds as two separated regio-isomers. Regiochemistry was assigned using NOE.

The second eluted regio-isomer was a yellow solid (0.5 g, 26% yield); ¹H NMR (400 MHz, d₆-DMSO): δ ppm 8.96 (s, 1H), 7.99 (d, J=6.4 Hz, 1H), 7.71-7.69 (m, 2H), 7.62-7.58 (m, 2H), 5.83 (s, 2H). LCMS-02 (Method-A): Rt 2.37 min, m/z 374.3 [M+H]⁺.

The first eluted regio-isomer was a yellow solid (1.0 g, 53% yield); ¹H NMR (400 MHz, d₆-DMSO): δ ppm 8.33 (s, 1H), 8.21 (d, J=6.0 Hz, 1H), 7.95 (d, J=6.0 Hz, 1H), 7.65 (d, J=8.4 Hz, 1H), 7.57 (s, 1H), 7.42 (d, J=9.2 Hz, 1H), 5.84 (s, 2H). LCMS-02 (Method-A): 2.51 min, m/z 374.3 [M+H]⁺.

### Intermediate 26: Synthesis of 4-chloro-2-(3-chloro-5-fluorobenzyl)-2H-pyrazolo[4,3-c]pyridine

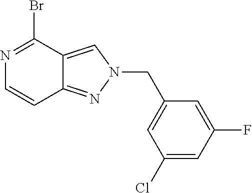 [SMILES: Fc1cc(Cl)cc(Cn2cc3c(Br)nccc3n2)c1]

To a stirred solution of 4-chloro-2H-pyrazolo[4,3-c]pyridine (CAS: 871836-51-0) (2.0 g, 13 mmol, 1.0 equiv) and 1-(bromomethyl)-3-chloro-5-fluorobenzene (CAS: 493024-39-8) (3.49 g, 15.7 mmol, 1.2 equiv) in DMF (20 mL), was added K₂CO₃ (5.41 g, 39.2 mmol, 3.0 equiv) and the reaction mixture was stirred at 120° C. for 2 h. After completion of the reaction as indicated by TLC (30% EtOAc in hexane), the reaction mixture was poured into ice-cold water (350 mL) and extracted with EtOAc (3× 200 mL). The organic layers were dried over Na₂SO₄ and concentrated under reduced pressure to afford the crude compound. The crude was purified by normal phase column chromatography (230-400 silica, gradient 0-9% EtOAc in hexane) to afford the title compounds as two separated regio-isomers. Regiochemistry was assigned using NOE.

The second eluting isomer was white solid, (0.51 g, 13% yield). ¹H NMR (400 MHz, d₆-DMSO): δ ppm 9.00 (s, 1H), 8.00 (d, J=6.4 Hz, 1H), 7.58 (d, J=6.4 Hz, 1H), 7.43 (d, J=8.8 Hz, 1H), 7.36 (s, 1H), 7.27 (d, J=8.8 Hz, 1H), 5.73 (s, 2H); LCMS-02 (Method-A): Rt 2.09 min, m/z 295.7 [M+H]⁺.

The first eluting isomer, yellow solid, (1.5 g, 38% yield); ¹H NMR (400 MHz, do-DMSO): δ ppm 8.41 (s, 1H), 8.22 (d, J=6.0 Hz, 1H), 7.90 (d, J=6.0 Hz, 1H), 7.38 (d, J=8.8 Hz, 1H), 7.21 (s, 1H), 7.10 (d, J=8.8 Hz, 1H), 5.76 (s, 2H), LCMS-02 (Method-A): Rt 2.23 min, m/z 295.7 [M+H]⁺.

### Intermediate 27: Synthesis of 1-methoxy-6,7-dihydroimidazo[1,5-a]pyrazin-8(5H)-one

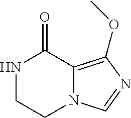

Step 1: To a stirred solution of ethyl 3-amino-3-oxopropanoate (CAS: 7597-56-0) (10.0 g, 76.3 mmol, 1.0 eq) in AcOH (100 mL) at room temperature, was added Br₂ (12.1 g, 76.3 mmol, 1.0 eq) at 60° C. and the reaction mixture was heated at 60° C. for 5 h. After completion of the reaction as indicated by TLC (50% EtOAc in hexane as a mobile phase), the solvent was removed under vacuum to get crude compound. The crude compound was purified by normal phase column chromatography (230-400 Silica, gradient 0-45% EtOAc in hexane) to afford ethyl 3-amino-2-bromo-3-oxopropanoate as a white solid (5.0 g, 31% yield). ¹H NMR (400 MHz, d₆-DMSO): δ ppm 7.85 (brs, 1H), 7.60 (brs, 1H), 5.10 (s, 1H), 4.16 (q, J=6.8 Hz, 2H), 1.19 (t, J=6.8 Hz, 3H). LCMS: Rt 1.01 min, m/z 209.8 [M+H]⁺.

Step 2: To a stirred solution of ethyl 3-amino-2-bromo-3-oxopropanoate (5.0 g, 24 mmol, 1.0 eq) in EtOH (150 mL) at room temperature was added benzyl(2-aminoethyl) carbamate (CAS: 72080-83-2) (4.62 g, 23.8 mmol, 1.0 eq) and triethylamine (4.81 g, 47.6 mmol, 2.0 eq). The reaction mixture was heated at 80° C. for 6 h. After completion of the reaction as indicated by TLC (10% MeOH in DCM as a mobile phase), the reaction mixture was partitioned between water (100 mL) and EtOAc (3×80 mL). The combined organic layers were dried in vacuo to get the crude material. The crude was purified by column chromatography (230-400 Silica, gradient 0-4% MeOH in DCM) to afford ethyl 3-amino-2-((2-(((benzyloxy)carbonyl)amino)ethyl)amino)-3-oxopropanoate as a pale-yellow solid (2.6 g, 33% yield). ¹H NMR (400 MHz, d₆-DMSO): δ ppm 7.60 (s, 1H), 7.38-7.29 (m, 8H), 5.01 (s, 2H), 4.13-4.06 (m, 2H), 3.84 (s, 1H), 3.09-3.07 (m, 2H), 2.58-2.54 (m, 1H), 2.47 (s, 1H), 1.18 (t, J=6.8 Hz, 3H). LCMS: Rt 1.15 min, m/z 323.9 [M+H]⁺.

Step 3: To a stirred solution of ethyl 3-amino-2-((2-(((benzyloxy)carbonyl)amino) ethyl)amino)-3-oxopropanoate (2.6 g, 8.0 mmol, 1.0 eq) in EtOH (80 mL) at room temperature was added triethyl orthoformate (7.14 g, 48.2 mmol, 6.0 eq) and p-toluene sulfonic acid (0.138 g, 0.804 mmol, 0.1 eq) at room temperature and the reaction mixture was heated at 80° C. for 6 h. After completion of the reaction as indicated by TLC (10% MeOH in DCM as a mobile phase), the reaction mixture was partitioned between water (60 mL) and DCM (3×50 mL). The combined organic layers were dried in vacuo. The resulting crude material was purified by normal phase column chromatography (230-400 silica, gradient 0-4.5% MeOH in DCM) to afford ethyl 1-(2-(((benzyloxy)carbonyl)amino)ethyl)-4-hydroxy-1H-imidazole-5-carboxylate as an off-white solid (1.10 g, 41% yield). ¹H NMR (400 MHz, d6-DMSO): δ ppm 10.71 (brs, 1H), 7.50 (s, 1H), 7.38-7.29 (m, 6H), 4.99 (s, 2H), 4.19-4.13 (m, 4H), 3.32-3.28 (m, 2H), 1.23 (t, J=6.8 Hz, 3H). LCMS: Rt 1.19 min, m/z 333.9 [M+H]⁺.

Step 4: To a stirred solution of ethyl 1-(2-(((benzyloxy)carbonyl)amino)ethyl)-4-hydroxy-1H-imidazole-5-carboxylate (1.0 g, 2.99 mmol, 1.0 equiv) in DMF (10 mL) at room temperature were added K₂CO₃ (1.24 g, 8.99 mmol, 3.0 equiv) and MeI (0.85 g, 5.99 mmol, 2.0 equiv) at room temperature and the reaction mixture was heated at 80° C. for 6 h. After completion of the reaction as indicated by TLC (10% MeOH in DCM as a mobile phase), the reaction mixture was poured into water (100 mL) and extracted with EtOAc (3× 40 mL). The combined organic layers were dried over Na₂SO₄ and concentrated under reduced pressure to get the crude material. The crude compound obtained was purified by normal phase column chromatography (230-400 silica, gradient 0-1% MeOH in DCM) to afford ethyl 1-(2-(((benzyloxy)carbonyl)amino)ethyl)-4-methoxy-1H-imidazole-5-carboxylate as an off-white solid (0.6 g, 57% yield). ¹H NMR (400 MHz, d₆-DMSO): δ ppm 7.49 (s, 1H), 7.38-7.29 (m, 6H), 4.99 (s, 2H), 4.24-4.16 (m, 4H), 3.86 (s, 3H), 3.31 (t, J=5.6 Hz, 2H), 1.25-1.16 (m, 3H). LCMS: Rt 1.47 min, m/z 348.1 [M+H]⁺.

Step 5: To a stirred solution of ethyl 1-(2-(((benzyloxy)carbonyl)amino)ethyl)-4-methoxy-1H-imidazole-5-carboxylate (0.6 g, 1.7 mmol, 1.0 equiv) in MeOH (60 mL) at room temperature was added 10% Pd/C (0.060 g, 10% w/w). The flask was filled with N₂ first and replaced with a hydrogen atmosphere using an H₂ balloon. The reaction mixture was vigorously stirred at room temperature for 24 h. After completion of the reaction as indicated by TLC (10% MeOH in DCM as a mobile phase), the reaction mixture was filtered through a Celite bed and the bed was washed with MeOH (20 mL). The obtained filtrate was concentrated under reduced pressure to afford ethyl 1-(2-aminoethyl)-4-methoxy-1H-imidazole-5-carboxylate as an off-white solid (0.4 g, Quantitative yield); ¹H NMR (400 MHz, d₆-DMSO): δ ppm 7.58 (s, 1H), 4.21-4.17 (m, 2H), 4.16-4.10 (m, 2H), 3.86 (s, 3H), 2.78-2.75 (m, 2H), 1.24 (t, J=3.6 Hz, 3H). LCMS: Rt 0.95 min, m/z 213.9 [M+H]⁺.

Step 6: To a stirred solution of ethyl 1-(2-aminoethyl)-4-methoxy-1H-imidazole-5-carboxylate (0.4 g, 1.9 mmol, 1.0 equiv) in toluene (4.0 mL) at room temperature was added trimethylaluminum (1.0 M solution in toluene) (3.75 mL, 3.75 mmol, 2.0 equiv) at room temperature and the reaction mixture was heated at 80° C. for 16 h. After completion of the reaction as indicated by TLC (10% MeOH in DCM as a mobile phase), the reaction mixture was diluted with a MeOH (50 mL) and filtered through a Celite bed. The filtrate was concentrated under a vacuum to get the crude material. The obtained crude material was purified by normal phase column chromatography (230-400 silica, gradient 0-4% MeOH in DCM) to afford 1-methoxy-6,7-dihydroimidazo[1,5-a]pyrazin-8(5H)-one as a pale yellow solid (0.17 g, 54% yield). ¹H NMR (400 MHz, d₆-DMSO): δ ppm 7.50 (s, 2H), 4.08 (t, J=5.2 Hz, 2H), 3.83 (s, 3H), 3.40 (brs, 2H). LCMS: Rt 0.55 min, m/z 168.1 [M+H]⁺.

### Intermediate 28: Synthesis of 4-bromo-2-(3-(difluoromethyl)-5-fluorobenzyl)-2H-pyrazolo[4,3-c]pyridine

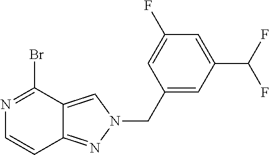 [SMILES: Fc1cc(Cn2cc3c(Br)nccc3n2)cc(C(F)F)c1]

To a stirred solution of 4-bromo-2H-pyrazolo[4,3-c]pyridine (CAS: 1159829-63-6) (1.5 g, 7.7 mmol, 1.0 equiv.), 1-(chloromethyl)-3-(difluoro methyl)-5-(1.67 g, 8.47 mmol, 1.1 equiv.) and K₂CO₃ (2.12 g, 15.4 mmol, 2.0 equiv.) in DMA (15 mL) was stirred at 110° C. for 3 h. After completion of the reaction as indicated by TLC (30% EtOAc in hexane) the reaction mixture was diluted with water (50.0 mL) and extracted with EtOAc (3×50 mL). The organic layers were dried over Na₂SO₄ and concentrated under reduced pressure to obtain the crude compound. The crude was purified by normal phase column chromatography (230-400 silica, gradient 0-35% EtOAc in hexane) to afford the two separated regio-isomers. Regiochemistry was confirmed by NOE analysis.

4-Bromo-2-(3-(difluoromethyl)-5-fluorobenzyl)-2H-pyrazolo[4,3-c]pyridine. 0.4 g, 14% yield, white solid. ¹H NMR (400 MHz, d₆-DMSO): δ ppm 8.95 (s, 1H), 7.98 (d, J=6.0 Hz, 1H), 7.61 (d, J=6.4 Hz, 1H), 7.45 (t, J=12.0 Hz, 3H), 7.05 (t, J=55.6 Hz, 1H), 5.80 (s, 2H). LCMS-08 (Method-A): Rt 2.13 min, MS (ES+) m/z 358.00 [M+H]⁺ for ⁸¹Br.

4-Bromo-1-(3-(difluoromethyl)-5-fluorobenzyl)-1H-pyrazolo[4,3-c]pyridine. 1.1 g, 40% yield, pale yellow solid. ¹H NMR (400 MHz, d₆-DMSO): δ ppm 8.33 (s, 1H), 8.20 (d, J=6.0 Hz, 1H), 7.93 (dd, J=5.6 Hz, 0.4 Hz, 1H), 7.39-7.30 (m, 3H), 7.01 (t, J=55.6 Hz, 1H), 5.81 (s, 2H). LCMS-08 (Method-A): Rt 2.52 min, MS (ES+) m/z 357.99 [M+H]⁺ for ⁸¹Br.

### Intermediate 29: Synthesis of 2-bromo-4-(3-fluoro-(trifluoromethyl)benzyl)pyridine

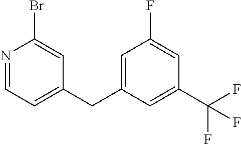 [SMILES: Fc1cc(Cc2ccnc(Br)c2)cc(C(F)(F)F)c1]

To a stirred solution of 1-(bromomethyl)-3-fluoro-5-(trifluoromethyl)benzene (CAS: 239087-09-3) (0.5 g, 1.9 mmol, 1.0 equiv) and (2-bromopyridin-4-yl) boronic acid (CAS: 458532-94-0) (0.39 g, 1.9 mmol, 1.0 equiv) in 1,4-dioxane (4.0 mL) and water (1.0 mL). The Na₂CO₃ (0.41 g, 3.9 mmol, 2.0 equiv) was added and the reaction mixture was purged with N₂ (g) for 10 min. The catalyst PdCl₂(dppf)·DCM complex (0.079 g, 0.097 mmol, 0.05 equiv) was added and the reaction mixture was heated at 90° C. for 3 h. After completion of the reaction, as indicated by TLC (30% EtOAc in hexane as a mobile phase), the reaction mixture was poured into water (15 mL) and extracted with EtOAc (3× 25 mL). The combined organic layers were dried over Na₂SO₄ and concentrated under reduced pressure to afford the crude compound. The crude was purified by normal phase flash column chromatography (100-200 silica, 0-25% EtOAc in hexane) to afford the title compound as a yellow oil (0.23 g, 35% yield); ¹H NMR (400 MHz, d₆-DMSO): δ ppm 8.30 (d, J=4.8 Hz, 1H), 7.66 (s, 1H), 7.61 (s, 1H), 7.56 (t, J=8.4 Hz, 2H), 7.38 (d, J=4.8 Hz, 1H), 4.10 (s, 2H); LCMS-02 (Method-A): Rt 2.61 min, m/z 334.3 [M+H]⁺.

### Intermediate 30: Synthesis of 2-chloro-6-(3-(difluoromethyl)-5-fluorobenzyl)pyridine

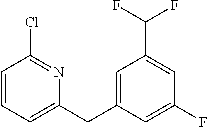 [SMILES: Fc1cc(Cc2cccc(Cl)n2)cc(C(F)F)c1]

To a stirred solution of 1-(chloromethyl)-3-(difluoromethyl)-5-fluorobenzene (0.4 g, 2 mmol, 1.0 equiv) in DMF (4.0 mL), was added 2-chloro-6-(4,4,5,5-tetramethyl-1,3,2-dioxaborolan-2-yl)pyridine (CAS: 652148-92-0) (0.738 g, 3.08 mmol, 1.5 equiv) and K₃PO₄ (0.871 g, 4.11 mmol, 2.0 equiv) at room temperature. The reaction mixture was purged with N₂ for 15 min, the catalyst Pd(PPh₃)₄ (0.118 g, 0.102 mmol, 0.05 equiv) was added and the sealed tube was heated up to 70° C. for 2 h. After completion of the reaction as indicated by TLC (10% EtOAc in hexane), the reaction mixture was diluted with water (50 mL) and extracted with EtOAc (3×50 mL). The combined organic layers were dried over Na₂SO₄ and concentrated under reduced pressure to afford the crude compound. The crude was purified by normal phase column chromatography (230-400 silica, gradient 4% EtOAc in hexane) to afford the title compound as yellow solid (0.22 g, 39% yield); ¹H NMR (400 MHz, d₆-DMSO): δ ppm 7.82 (t, J=7.6 Hz, 1H), 7.39-7.29 (m, 5H), 7.02 (t, J=55.2 Hz, 1H), 4.17 (s, 2H). LCMS-02 (Method-A): Rt 2.50 min, m/z 272.4 [M+H]⁺.

### Intermediate 31: Synthesis of 1-ethoxy-6,7-dihydroimidazo[1,5-a]pyrazin-8(5H)-one

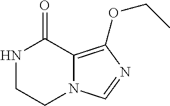

Step 1: To a stirred solution of ethyl 1-(2-(((benzyloxy)carbonyl)amino)ethyl)-4-hydroxy-1H-imidazole-5-carboxylate (1.1 g, 3.3 mmol, 1.0 equiv) in DMF (22 mL), was added K₂CO₃ (1.36 g, 9.90 mmol, 3.0 equiv) and ethyl iodide (1.02 g, 6.60 mmol, 2.0 equiv) at room temperature and the reaction mixture was heated at 80° C. for 8 h. After completion of the reaction as indicated by TLC (10% MeOH in DCM), the reaction mixture was poured into water (40 mL) and extracted with EtOAc (3× 20 mL). The combined organic layers were dried over Na₂SO₄ and concentrated under reduced pressure to afford the crude compound. The crude was purified by normal phase column chromatography (230-400 silica, gradient 0-1% MeOH in DCM) to afford ethyl 1-(2-(((benzyloxy)carbonyl)amino)ethyl)-4-ethoxy-1H-imidazole-5-carboxylate as an off white solid (0.7 g, 59% yield). ¹H NMR (400 MHz, d₆-DMSO): δ ppm 7.46 (s, 1H), 7.38-7.29 (m, 6H), 4.99 (s, 2H), 4.29-4.15 (m, 6H), 3.30 (s, 2H, obscured by DMSO moisture peak), 1.30-1.22 (m, 6H). LCMS-02 (Method-A): Rt 2.10 min, m/z 362.5 [M+H]⁺.

Step 2: To a stirred solution of ethyl 1-(2-(((benzyloxy)carbonyl)amino)ethyl)-4-ethoxy-1H-imidazole-5-carboxylate (0.7 g, 1.9 mmol, 1.0 equiv) in MeOH (7 mL), was added 10% Pd/C (0.070 g, 10% w/w) at room temperature. The flask was filled with N₂ first and replaced with a hydrogen atmosphere using an H₂ balloon. The reaction mixture was vigorously stirred at room temperature for 24 hours. After completion of the reaction as indicated by TLC (10% MeOH in DCM), the reaction mixture was filtered through a Celite bed and the bed was washed with MeOH (20 mL). The obtained filtrate was concentrated under reduced pressure to afford ethyl 1-(2-aminoethyl)-4-ethoxy-1H-imidazole-5-carboxylate as an off-white solid (0.4 g, 91% yield); ¹H NMR (400 MHz, d₆-DMSO): δ ppm 7.58 (s, 1H), 4.27 (q, J=6.8 Hz, 2H), 4.21-4.15 (m, 4H), 3.63 (brs, 2H), 2.89-2.85 (m, 2H), 1.30-1.22 (m, 6H).; LCMS-02 (Method-A): Rt 1.48 min, m/z 228.3 [M+H]⁺.

Step 3: To a stirred solution of ethyl 1-(2-aminoethyl)-4-ethoxy-1H-imidazole-5-carboxylate (0.4 g, 1.76 mmol, 1.0 equiv) in toluene (4.0 mL), was added TMA (2.0 M solution in toluene) (1.76 mL, 3.52 mmol, 2.0 equiv) at room temperature and the reaction mixture was heated at 80° C. for 16 h. After completion of the reaction as indicated by TLC (10% MeOH in DCM), the reaction mixture was diluted with MeOH (30 mL) and filtered through a Celite bed, the filtrate was concentrated under reduced pressure to afford the crude compound. The obtained crude material was purified by normal phase column chromatography (230-400 silica, gradient 2% MeOH in DCM) to afford 1-ethoxy-6,7-dihydroimidazo[1,5-a]pyrazin-8(5H)-one as an off-white solid (0.25 g, 78% yield). ¹H NMR (400 MHz, d₆-DMSO): δ ppm 7.48 (s, 2H), 4.25 (q, J=6.8, 2H), 4.07 (t, J=5.6 Hz, 2H), 3.42-3.38 (m, 2H), 1.27 (t, J=6.8 Hz, 3H). LCMS-02 (Method-A): Rt 0.67 min, m/z 182.2 [M+H]⁺.

### Intermediate 32: Synthesis of 2-chloro-4-((3-(difluoromethyl)-5-fluorophenyl)difluoromethyl)pyridine

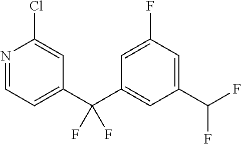 [SMILES: Fc1cc(C(F)F)cc(C(F)(F)c2ccnc(Cl)c2)c1]

Step 1: To a stirred solution of 2-chloro-4-(3-(difluoromethyl)-5-fluorobenzyl)pyridine (1.50 g, 5.52 mmol, 1.0 equiv) in acetic acid (15 mL), was added SeO₂ (1.83 g, 16.6 mmol, 3.0 equiv) at room temperature and the reaction mixture was heated at 100° C. for 9 h. After completion of the reaction as indicated by TLC (10% EtOAc in hexane as a mobile phase), the reaction mixture was quenched with saturated NaHCO₃ solution (100 mL) (pH ~7) and extracted with EtOAc (3×50 mL). The combined organic layers were dried over Na₂SO₄ and concentrated under reduced pressure to afford the crude compound. The crude was purified by normal phase column chromatography (230-400 silica, gradient 0-7% EtOAc in hexane) to afford (2-chloropyridin-4-yl) (3-(difluoromethyl)-5-fluorophenyl) methanone as a pale-yellow liquid (0.400 g, 25% yield). ¹H NMR (400 MHz, d₆-DMSO): δ ppm 8.66 (d, J=5.2 Hz, 1H), 7.87-7.78 (m, 4H), 7.68 (dd, J=1.2 Hz, 5.2 Hz, 1H), 7.17 (t, J=55.2 Hz, 1H); LCMS-08 (Method-A): Rt 2.15 min, m/z 286.1 [M+H]⁺.

Step 2: To a stirred solution of (2-chloropyridin-4-yl) (3-(difluoromethyl)-5-fluorophenyl) methanone (0.400 g, 1.40 mmol, 1.0 equiv) in DCM (4.0 mL) at 0° C., was added DAST (0.677 g, 4.20 mmol, 3.0 equiv) and the reaction mixture was stirred at room temperature for 16 h. After completion of the reaction as indicated by TLC (10% EtOAc in hexane as a mobile phase), the reaction mixture was quenched with saturated NaHCO₃ solution (50 mL) (pH ~7) and extracted with EtOAc (3×20 mL). The combined organic layers were dried over Na₂SO₄ and concentrated under reduced pressure to afford the crude compound. The crude was purified by normal phase column chromatography (230-400 silica, gradient 0-5% EtOAc in hexane) to afford 2-chloro-4-((3-(difluoromethyl)-5-fluorophenyl) difluoromethyl)pyridine as a light yellow liquid (0.220 g, 51% yield). ¹H NMR (400 MHz, do-DMSO): δ ppm 8.60 (d, J=5.2 Hz, 1H), 7.90 (s, 1H), 7.82 (d, J=9.2, 1H), 7.73-7.71 (m, 2H), 7.68 (d, J=8.8 Hz, 1H), 7.10 (t, J=55.2 Hz, 1H); LCMS-02 (Method-A): Rt 2.49 min, m/z 308.0 [M+H]⁺.

### Intermediate 33: Synthesis of 4-chloro-6-(3-fluoro-5-(trifluoromethyl)benzyl)pyrimidine

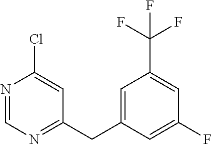 [SMILES: Fc1cc(Cc2cc(Cl)ncn2)cc(C(F)(F)F)c1]

To a stirred solution of 1-(bromomethyl)-3-fluoro-5-(trifluoromethyl)benzene (CAS: 239087-09-3) (1.00 g, 3.89 mmol, 1.0 equiv) in THF (10 mL), was added Zn metal (0.508 g, 7.78 mmol, 2.0 equiv) at room temperature and the reaction mixture was stirred at 80° C. for 30 min. The reaction mixture was allowed to cool to room temperature then 4,6-dichloropyrimidine (CAS: 1193-21-1) (1.15 g, 7.78 mmol, 2.0 equiv) and catalyst PdCl₂(PPh₃)₂ (0.272 g, 0.389 mmol, 0.1 equiv) were added and the reaction mixture was stirred again at 80° C. for 16 h. After completion of the reaction as indicated by TLC (30% EtOAc in hexane), the reaction mixture was filtered through a Celite bed, and the bed was washed with EtOAc (2×10 mL). The filtrate was diluted with water (20 mL) and extracted with EtOAc (2×20 mL). The organic layers were dried over Na₂SO₄ and concentrated under reduced pressure to afford the crude compound. The crude was purified by normal phase column chromatography (230-400 silica, gradient 0-15% EtOAc in hexane) to afford 4-chloro-6-(3-fluoro-5-(trifluoromethyl)benzyl) pyrimidine as a yellow oil (0.13 g, 12% yield); ¹H NMR (400 MHz, d₆-DMSO): δ ppm 8.97 (s, 1H), 7.77 (s, 1H), 7.61 (s, 1H), 7.56 (t, J=8.4 Hz, 2H), 4.26 (s, 2H); LCMS-02 (Method-A): Rt 2.45 min, m/z 291.0 [M+H]⁺.

### Intermediate 34: Synthesis of 1-(difluoro methyl)-6,7-dihydroimidazo[1,5-a]pyrazin-8(5H)-one

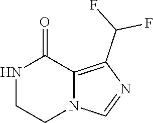 [SMILES: O=C1NCCn2cnc(C(F)F)c21]

Step 1: To a stirred solution of dimethyl 1H-imidazole-4,5-dicarboxylate (CAS: 3304-70-9) (40.0 g, 217 mmol, 1.0 equiv) and benzyl(2-hydroxyethyl)carbamate (CAS: 77987-49-6) (63.6 g, 326 mmol, 1.5 equiv) in THF (400 mL) at −40° C. under nitrogen atmosphere. The triphenylphosphine (85.5 g, 326 mmol, 1.5 equiv) was added portion wise then DIAD (87.8 g, 434 mmol, 2.0 equiv) was also added dropwise to the reaction mixture. The resulting reaction mixture was stirred at RT for 16 h. The completion of the reaction was indicated by TLC (100% EtOAc as mobile phase), and the reaction mixture was poured into ice-cold water (500 mL) and extracted with EtOAc (3×500 mL). The organic layers were dried over Na₂SO₄ and concentrated under reduced pressure to afford the crude compound. The crude was purified by normal phase column chromatography (60-120 silica, gradient 0-15% MeOH in DCM) to afford dimethyl 1-(2-(((benzyloxy)carbonyl)amino)ethyl)-1H-imidazole-4,5-dicarboxylate as a yellow gummy liquid (40.0 g, 51% yield). ¹H NMR (400 MHz, d₆-DMSO): δ ppm 7.86 (s, 1H), 7.41-7.29 (m, 6H), 4.98 (s, 2H), 4.25 (t, J=5.2 Hz, 2H), 3.80 (s, 3H), 3.78 (s, 3H), 3.32 (2H obscured by DMSO moisture peak). LCMS-02 (Method-A): Rt 1.83 min, m/z 362.3 [M+H]⁺.

Step 2: To a stirred solution of dimethyl 1-(2-(((benzyloxy)carbonyl)amino)ethyl)-1H-imidazole-4,5-dicarboxylate (40.0 g, 111 mmol, 1.0 equiv) in methanol (400 mL) was added palladium on carbon (4.0 g, 10% w/w) portion wise and the reaction mixture was stirred at rt under H₂ atmosphere for 16 h. The completion of the reaction was monitored by TLC (10% methanol in DCM as mobile phase). The resulting reaction mixture was filtered through a celite bed and the bed was washed with methanol (1000 mL). The combined filtrate was concentrated under reduced pressure to afford the crude compound. The crude was triturated with EtOAc to afford methyl 8-oxo-5,6,7,8-tetrahydroimidazo[1,5-a]pyrazine-1-carboxylate as a white solid (17.0 g, 79% yield). ¹H NMR (400 MHz, d₆-DMSO): δ ppm 8.17 (brs, 1H), 7.89 (s, 1H), 4.21 (t, J=5.6 Hz, 2H), 3.75 (s, 3H), 3.51-3.47 (m, 2H). LCMS-02 (Method-A): Rt 0.34 min, m/z 196.1 [M+H]⁺.

Step 3: To a stirred solution of methyl 8-oxo-5,6,7,8-tetrahydroimidazo[1,5-a] pyrazine-1-carboxylate (10.0 g, 51.2 mmol, 1.0 equiv) in DMF (100 mL) at 0° C., was added NaH (60%), 1.84 g, 76.9 mmol, 1.5 equiv) and the reaction mixture was stirred at the same temperature for 10 min. 4-Methoxybenzyl chloride (CAS: 824-94-2) (9.62 g, 61.5 mmol, 1.2 equiv) was added dropwise in the reaction mixture, and the reaction mixture was stirred at 0° C. to RT for 6 h. After completion of the reaction as indicated by TLC (10% MeOH in DCM as a mobile phase), The reaction mixture was poured into cold water (300 mL) and extracted with EtOAc (3×100 mL). The combined organic layers were dried over sodium sulphate and concentrated under reduced pressure to afford the crude compound. The crude compound was purified by normal phase column chromatography (100-200 silica, gradient 0-7% MeOH in DCM) to afford the title compound methyl 7-(4-methoxybenzyl)-8-oxo-5,6,7,8-tetrahydroimidazo[1,5-a]pyrazine-1-carboxylate as a white solid (7.0 g, 43% yield). ¹H NMR (400 MHz, d₆-DMSO): δ 7.87 (s, 1H), 7.25 (d, J=8.8 Hz, 2H), 6.91 (d, J=8.8 Hz, 2H), 4.59 (s, 2H), 4.22 (t, J=5.6 Hz, 2H), 3.77 (s, 3H), 3.74 (s, 3H), 3.61 (t, J=6.0 Hz, 2H). LCMS-02-(Method-A): Rt 1.64 min, m/z 316.3 [M+H]⁺.

Step 4: Two parallel batches (3.5 g×2) were performed: To each stirred solution of methyl 7-(4-methoxybenzyl)-8-oxo-5,6,7,8-tetrahydroimidazo[1,5-a]pyrazine-1-carboxylate (3.5 g, 11 mmol, 1.0 equiv) in THF (175 mL) at 0° C., was added LiBH₄ (1.0 M solution in THF) (33.3 mL, 33.3 mmol, 3.0 equiv), then the reaction mixtures were stirred at RT for 6 h. After completion of the reactions as indicated by TLC (10% MeOH in DCM as a mobile phase), the reaction mixtures were quenched with 1N HCl to adjust the pH up to ~6 and extracted with EtOAc (3×100 mL). The combined organic layers were dried over sodium sulphate. Organic layers from both batches were combined and concentrated under reduced pressure to afford the crude compound. The crude was purified by normal phase column chromatography (silica 230-400 mesh, 0-10% MeOH in DCM) to afford 1-(hydroxymethyl)-7-(4-methoxybenzyl)-6,7-dihydroimidazo[1,5-a]pyrazin-8(5H)-one as a white solid (4.0 g, 63% combined yield across 2 parallel batches). ¹H NMR (400 MHz, d₆-DMSO): δ 7.73 (s, 1H), 7.25 (d, J=8.8 Hz, 2H), 6.91 (d, J=8.8 Hz, 2H), 5.27 (t, J=6.0 Hz, 1H), 4.61-4.60 (m, 4H, Two-CH₂ merged together), 4.19 (t, J=6.4 Hz, 2H), 3.73 (s, 3H), 3.57 (t, J=6.0 Hz, 2H). LCMS-02 (Method-A): Rt 1.51 min, m/z 288.4 [M+H]⁺.

Step 5: Note: Two parallel batches (2.0 g×2) were performed: To each stirred solution of 1-(hydroxymethyl)-7-(4-methoxybenzyl)-6,7-dihydroimidazo[1,5-a]pyrazin-8(5H)-one (2.0 g, 7.0 mmol, 1.0 equiv) in DCM (40.0 mL) was added MnO₂ (5.98 g, 69.6 mmol, 10.0 equiv) and the reaction mixtures stirred at RT for 8 h. After completion of the reactions as indicated by TLC (5% MeOH in DCM as a mobile phase), the reaction mixtures were filtered through a celite bed and the filtrate collected. The filtrates from both reactions were combined and concentrated under reduced pressure. The crude was purified by normal phase column chromatography (230-400 silica, gradient 0-8% MeOH in DCM) to afford 7-(4-methoxybenzyl)-8-oxo-5,6,7,8-tetrahydroimidazo[1,5-a]pyrazine-1-carbaldehyde as a pale-yellow solid (2.5 g, 63% combined yield across 2 parallel batches). ¹H NMR (400 MHz, d₆-DMSO): δ 10.37 (s, 1H), 8.00 (s, 1H), 7.29 (d, J=8.8 Hz, 2H), 6.92 (d, J=8.4 Hz, 2H), 4.65 (s, 2H), 4.30 (t, J=5.6 Hz, 2H), 3.74 (s, 3H), 3.68-3.65 (m, 2H). LCMS-02 (Method-A): Rt 1.67 min, m/z 286.3 [M+H]⁺.

Step 6: To a stirred solution of 7-(4-methoxybenzyl)-8-oxo-5,6,7,8-tetrahydroimidazo[1,5-a]pyrazine-1-carbaldehyde (2.5 g, 8.8 mmol, 1.0 equiv.) in DCM (25.0 mL) at −40° C., was added DAST (4.32 g, 26.3 mmol, 3.0 equiv.) dropwise and the reaction mixture was stirred at −40° C. for 4 h. After completion of the reaction as indicated by TLC (10% MeOH in DCM as a mobile phase), the reaction mixture was poured into 10% aq. NaHCO₃ solution (50 mL) and extracted with EtOAc (3× 40 mL). The combined organic layers were dried over sodium sulphate and concentrated under reduced pressure to afford the crude compound. The crude was purified by normal phase column chromatography (silica 100-200, gradient 5-10% MeOH in DCM) to afford 1-(difluoro methyl)-7-(4-methoxybenzyl)-6,7-dihydroimidazo[1,5-a]pyrazin-8(5H)-one as a pale-yellow solid (0.5 g, 19% yield). ¹H NMR (400 MHz, d₆-DMSO): δ 7.93 (s, 1H), 7.27 (d, J=8.8 Hz, 3H), 6.91 (d, J=8.8 Hz, 2H), 4.60 (s, 2H), 4.26 (t, J=5.6 Hz, 2H), 3.73 (s, 3H), 3.62 (t, J=6.0 Hz, 2H). LCMS-02 (Method-A): Rt 1.83 min, m/z 308.3 [M+H]⁺.

Step 7: To a stirred solution of 1-(difluoro methyl)-7-(4-methoxybenzyl)-6,7-dihydroimidazo[1,5-a]pyrazin-8(5H)-one (0.500 g, 1.63 mmol, 1.0 equiv) in TFA (5.0 mL) at RT, was added triflic acid (2.5 mL, 5.0 v) dropwise, then reaction mixture was stirred at 70° C. for 4 h. After completion of the reaction as indicated by TLC (using 20% MeOH in DCM as a mobile phase), the reaction mixture was poured into a aq. saturated bicarbonate solution (50 mL) and concentrated under reduced pressure to afford the crude (0.6 g). The crude compound was purified by normal phase column chromatography (silica gel 100-200, 10-15% MeOH in DCM) to afford the title compound 1-(difluoro methyl)-6,7-dihydroimidazo[1,5-a]pyrazin-8(5H)-one as a pale-yellow solid (0.180 g, 59% yield); ¹H NMR (400 MHz, d₆-DMSO): δ 8.29 (brs, 1H), 7.95 (s, 1H), 7.21 (t, J=54.0 Hz, 1H), 4.23 (t, J=6.0 Hz, 2H), 3.55-3.51 (m, 2H). LCMS-02-Method-A: Rt 0.552 min, m/z 188.13 [M+H]⁺.

### Intermediate 35: Synthesis of 2-chloro-4-(3-fluoro-4-(trifluoromethyl)benzyl)pyridine

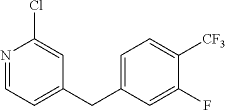 [SMILES: Fc1cc(Cc2ccnc(Cl)c2)ccc1C(F)(F)F]

Step 1: To a stirred solution of (3-fluoro-4-(trifluoromethyl)phenyl) methanol (CAS: 230295-16-6) (1.00 g, 5.15 mmol, 1.0 equiv) in CHCl₃ (10 mL), was added SOCl₂ (10 mL) at room temperature and the reaction mixture was stirred at 70° C. for 16 h. After completion of the reaction as indicated by TLC (10% EtOAc in hexane), the reaction mixture was concentrated under reduced pressure to afford the crude 4-(chloromethyl)-2-fluoro-1-(trifluoromethyl)benzene as a brownish liquid (0.7 g, 64% yield); ¹H NMR (400 MHz, d₆-DMSO): δ ppm 7.81 (t, J=8.0 Hz, 1H), 7.61 (d, J=11.6 Hz, 1H), 7.50 (d, J=8 Hz, 1H), 4.85 (s, 2H).

Step 2: To a stirred solution of 4-(chloromethyl)-2-fluoro-1-(trifluoromethyl)benzene (0.7 g, 3.3 mmol, 1.0 equiv) and (2-chloropyridin-4-yl) boronic acid (CAS: 458532-96-2) (0.570 g, 3.62 mmol, 1.1 equiv) in 1,4-dioxane (5.6 mL) and water (1.4 mL), was added K₂CO₃ (0.908 g, 6.59 mmol, 2.0 equiv.), then the reaction mixture was purged with N₂ for 10 min. The catalyst PdCl₂(dppf)·DCM complex (0.134 g, 0.164 mmol, 0.05 equiv) was added and the reaction mixture was heated at 90° C. for 2 h. After completion of the reaction as indicated by TLC (20% EtOAc in hexane), the reaction mixture was diluted with water (40 mL) and extracted with EtOAc (3× 20 mL). The combined organic layers were dried over Na₂SO₄ and concentrated under reduced pressure to afford the crude compound. The crude was purified by normal phase column chromatography (230-400 silica, gradient 0-12% EtOAc in hexane) to afford 2-chloro-4-(3-fluoro-4-(trifluoromethyl)benzyl)pyridine as a light yellow liquid (0.25 g, 26% yield); ¹H NMR (400 MHz, d₆-DMSO): δ ppm 8.33 (dd, J=4.4 & 0.8 Hz, 1H), 7.74 (t, J=2.0 Hz, 1H), 7.53-7.50 (m, 2H), 7.36-7.33 (m, 2H), 4.11 (s, 2H); LCMS-07 (Method-A): Rt 2.47 min, m/z 290.1 [M+H]⁺.

### Intermediate 36: Synthesis of 2-chloro-4-(4-(difluoromethyl)-3-fluorobenzyl)pyridine

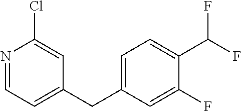 [SMILES: Fc1cc(Cc2ccnc(Cl)c2)ccc1C(F)F]

Step 1: To a stirred solution of methyl 3-fluoro-4-formylbenzoate (CAS: 74733-25-8) (2.0 g, 11.0 mmol, 1.0 equiv) in DCM (20 mL) at 0° C., was added DAST (5.30 g, 32.9 mmol, 3.0 equiv) and the reaction mixture was stirred at room temperature for 6 h. After completion of the reaction as indicated by TLC (20% EtOAc in hexane), the reaction mixture was quenched with saturated NaHCO₃ solution (50 mL) and extracted with DCM (3× 30 mL). The combined organic layers were dried over Na₂SO₄ and concentrated under reduced pressure to afford the crude compound. The crude was purified by normal phase column chromatography (230-400 silica, gradient 0-10% EtOAc in hexane) to afford methyl 4-(difluoromethyl)-3-fluorobenzoate as a light-yellow liquid (1.0 g, 45% yield); ¹H NMR (400 MHz, d₆-DMSO): δ ppm 7.92-7.90 (m, 1H), 7.85-7.79 (m, 2H), 7.30 (t, J=54.8 Hz, 1H), 3.89 (s, 3H).

Step 2: To a stirred solution of methyl 4-(difluoromethyl)-3-fluorobenzoate (1.0 g, 4.9 mmol, 1.0 equiv) in THF (10 mL) at 0° C., was added LiAlH₄ (2.4 mL) slowly and the reaction mixture was stirred at room temperature for 2 h. After completion of the reaction as indicated by TLC (20% EtOAc in hexane), the reaction mixture was quenched with 1 N HCl solution (30 mL) and extracted with EtOAc (3×20 mL). The combined organic layers were dried over Na₂SO₄ and concentrated under reduced pressure to afford (4-(difluoromethyl)-3-fluorophenyl) methanol as a colourless liquid (0.8 g, 93% yield); ¹H NMR (400 MHz, do-DMSO): δ ppm 7.59 (t, J=8.0 Hz, 1H), 7.32-7.04 (m, 3H; CHF₂ proton signal merge), 5.46 (t, J=6.0 Hz, 1H), 4.57 (d, J=5.6 Hz, 2H).

Step 3: To a stirred solution of (4-(difluoromethyl)-3-fluorophenyl) methanol (0.8 g, 4.5 mmol, 1.0 equiv) in CHCl₃ (8 mL), was added SOCl₂ (8 mL) at room temperature and the reaction mixture was stirred at 70° C. for 16 h. After completion of the reaction as indicated by TLC (20% EtOAc in hexane), the reaction mixture was concentrated under reduced pressure and azeotrope with DCM (3× 50 mL) to afford 4-(chloromethyl)-1-(difluoromethyl)-2-fluorobenzene as a colourless liquid (0.6 g, 68% yield); ¹H NMR (400 MHz, d₆-DMSO): δ ppm 7.66 (t, J=7.6 Hz, 1H), 7.48-7.42 (m, 2H), 7.21 (t, J=54.0 Hz, 1H), 4.82 (s, 2H).

Step 4: To a stirred solution of 4-(chloromethyl)-1-(difluoromethyl)-2-fluorobenzene (0.60 g, 3.1 mmol, 1.0 equiv) and (2-chloropyridin-4-yl) boronic acid (CAS: 458532-96-2) (0.485 g, 3.08 mmol, 1.0 equiv) in 1,4-dioxane (4.2 mL) and water (1.8 mL), was added K₂CO₃ (1.27 g, 9.25 mmol, 3.0 equiv.) then the reaction mixture was purged with N₂ for 10 min. The PdCl₂(dppf)·DCM complex (0.125 g, 0.154 mmol, 0.05 equiv.) was added and the reaction mixture was heated at 90° C. for 2 h. After completion of the reaction as indicated by TLC (20% EtOAc in hexane), the reaction mixture was diluted with water (50 mL) and extracted with EtOAc (3× 15 mL). The combined organic layers were dried over Na₂SO₄ and concentrated under reduced pressure to afford the crude compound. The crude was purified by normal phase column chromatography (230-400 silica, gradient 0-15% EtOAc in hexane) to afford 2-chloro-4-(4-(difluoromethyl)-3-fluorobenzyl)pyridine as a colourless liquid (0.25 g, 30% yield); ¹H NMR (400 MHz, d₆-DMSO): δ ppm 8.32 (dd, J=5.2 & 0.8 Hz, 1H), 7.59 (t, J=7.6 Hz, 1H), 7.48-7.47 (m, 1H), 7.37 (d, J=11.6 Hz, 1H), 7.32 (dd, J=5.2 & 1.6 Hz, 1H), 7.30-7.03 (m, 2H), 4.07 (s, 2H); LCMS-02 (Method-A): Rt 2.47 min, m/z 272.1 [M+H]⁺.

### Intermediate 37: Synthesis of 4-bromo-2-(3-fluoro-5-(trifluoromethyl)benzyl)-5-methylpyridine

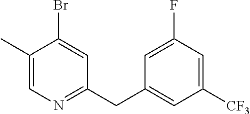 [SMILES: Cc1cnc(Cc2cc(F)cc(C(F)(F)F)c2)cc1Br]

Step 1: Preparation of aryl magnesium bromide solution: To an oven-dried 3 neck round-bottom flask was added magnesium turnings (39 mg, 1.60 mmol) and anhydrous THF (2 mL). A small piece of iodine was added to the solution as an initiator and the solution was heated to 65° C. under N₂ atmosphere. After 15 min, a solution of 1-bromo-3-fluoro-5-(trifluoromethyl)benzene (300 mg, 1.23 mmol) in THF (1 mL) was added dropwise. The reaction was kept at 65° C. until most of Mg disappeared, and then cooled to RT for use. To a solution of 4-bromo-5-methylpicolinaldehyde (198 mg, 0.99 mmol) in THF (5 mL) was added the freshly prepared BrMgAr solution at 0° C. under N₂. The solution was stirred at room temperature until the starting materials were consumed (2 h). The reaction was quenched by ammonium chloride solution and the mixture was extracted by EtOAc (3×30 mL). The combined organic layer was washed with brine, dried by Na₂SO₄ and concentrated in vacuo. The residue was purified by flash silica gel column chromatography eluting with EtOAc: petroleum ether (1:4) to afford (4-bromo-5-methylpyridin-2-yl) (3-fluoro-5-(trifluoromethyl)phenyl) methanol as a pale-yellow solid (185 mg, 51% yield). ¹H NMR (400 MHz, Acetone-d6) δ 8.39 (s, 1H), 7.85 (s, 1H), 7.71 (s, 1H), 7.58 (dt, J=9.6, 1.9 Hz, 1H), 7.38 (dt, J=8.7, 2.1 Hz, 1H), 5.96 (d, J=4.5 Hz, 1H), 5.66 (d, J=4.6 Hz, 1H), 2.35 (s, 3H). LC/MS (APCI): m/z=365.9 [M+H]⁺, Rt 4.025 min.

Step 2: To a solution of (4-bromo-5-methylpyridin-2-yl) (3-fluoro-5-(trifluoromethyl)phenyl) methanol (100 mg, 0.28 mmol) in THF (5 mL) was sequentially added with imidazole (47 mg, 0.69 mmol), PPh₃ (144 mg, 0.55 mmol), and iodine (140 mg, 0.55 mmol). The solution was heated to 60° C. for 12 h. Upon completion, the reaction mixture was quenched by adding water (10 mL), and extracted with EtOAc (3×30 mL). The combined organic phase was washed with brine, dried over Na₂SO₄ and concentrated in vacuo. The residue was purified by flash silica gel column chromatography eluting with EtOAc: petroleum ether (1:4) to afford the title compound as pale-yellow oil (54 mg, 56%). ¹H NMR (400 MHz, Acetone-d6) δ 8.40 (s, 1H), 7.64 (s, 1H), 7.56 (s, 1H), 7.46-7.43 (m, 1H), 7.37-7.34 (m, 1H), 4.23 (s, 2H), 2.34 (s, 3H), LC/MS (ESI): m/z=348.0 [M+H]⁺, Rt 4.362 min.

### Intermediate 38: Synthesis of 4-bromo-2-(3-fluoro-5-(trifluoromethyl)benzyl)-5-methoxypyridine

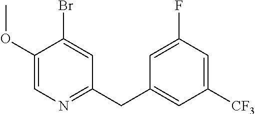 [SMILES: COc1cnc(Cc2cc(F)cc(C(F)(F)F)c2)cc1Br]

Step 1: Preparation of aryl magnesium bromide solution: To an oven-dried 3 neck round-bottom flash was added magnesium turnings (26 mg, 1.07 mmol) and anhydrous THF (2 mL). A small piece of iodine was added to the solution as an initiator and the solution was heated to 65° C. under N₂ atmosphere. After 15 min, a solution of 1-bromo-3-fluoro-5-(trifluoromethyl)benzene (200 mg, 0.82 mmol) in THF (1 mL) was added dropwise. The reaction was kept at 65° C. until most of Mg disappeared, and then cooled to RT for use. To a solution of 4-bromo-5-methoxypicolinaldehyde (142 mg, 0.66 mmol) in THF (4 mL) was added the freshly prepared BrMgAr solution at 0° C. under N₂. The solution was stirred at room temperature until the starting materials were consumed (2 h). The reaction was quenched by ammonium chloride solution and the mixture was extracted by EtOAc (3×30 mL). The combined organic layer was washed with brine, dried by Na₂SO₄ and concentrated in vacuo. The residue was purified by flash silica gel column chromatography eluting with EtOAc: petroleum ether (1:4) to afford (4-bromo-5-methoxypyridin-2-yl) (3-fluoro-5-(trifluoromethyl)phenyl) methanol as yellow oil (142 mg, 57% yield). ¹H NMR (400 MHz, Acetone-d6) δ 8.28 (s, 1H), 7.84 (s, 1H), 7.70-7.67 (m, 1H), 7.58-7.54 (m, 1H), 7.39-7.36 (m, 1H), 5.95 (d, J=4.6 Hz, 1H), 5.62 (d, J=4.6 Hz, 1H), 4.01 (s, 3H), LC/MS (ESI): m/z=379.9 [M+H]⁺, Rt 3.893 min.

Step 2: To a solution of (4-bromo-5-methoxypyridin-2-yl) (3-fluoro-5-(trifluoromethyl)phenyl) methanol (120 mg, 0.32 mmol) in THF (5 mL) was sequentially added with imidazole (54 mg, 0.79 mmol), PPh₃ (166 mg, 0.63 mmol), and iodine (160 mg, 0.63 mmol). The solution was heated to 60° C. for 8 h. Upon completion, the reaction mixture was quenched by adding water (10 mL), and extracted with EtOAc (3×30 mL). The combined organic phase was washed with brine, dried over Na₂SO₄ and concentrated in vacuo. The residue was purified by flash silica gel column chromatography eluting with

EtOAc: petroleum ether (1:4) to afford the title compound as pale-yellow oil (21 mg, 18%). ¹H NMR (400 MHz, Acetone-d6) δ 8.29 (s, 1H), 7.64 (s, 1H), 7.54 (s, 1H), 7.46-7.39 (m, 1H), 7.37-7.33 (m, 1H), 4.21 (s, 2H), 4.01 (s, 3H), LC/MS (APCI): m/z=363.9 [M+H]⁺, Rt 4.124 min.

### Intermediate 39: Synthesis of 6-((2-chloropyridin-4-yl)oxy)-1-methyl-1H-indazole

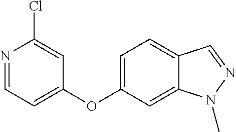 [SMILES: Cn1ncc2ccc(Oc3ccnc(Cl)c3)cc21]

To a solution of 1-methyl-1H-indazol-6-ol (100 mg, 0.68 mmol) and 2-chloro-4-fluoropyridine (107 mg, 0.81 mmol) in DMF (4 mL) was added K₂CO₃ (187 mg, 1.35 mmol) and the mixture was stirred at 80° C. for 8 h. Upon completion, water (20 mL) was added and the mixture was extracted with EtOAc (3×50 mL). The combined organic layer was washed with brine, dried over MgSO₄ and concentrated in vacuo. The residue was purified by flash silica gel column chromatography eluting with EtOAc:PET ether (1:1) to afford the title compound as a beige solid (142 mg, 81%). ¹H NMR (400 MHz, Acetone-d6) δ 8.27 (dd, J=5.5, 0.6 Hz, 1H), 8.04 (d, J=1.0 Hz, 1H), 7.88 (dd, J=8.6, 0.7 Hz, 1H), 7.47-7.46 (m, 1H), 7.01-6.93 (m, 3H), 4.07 (s, 3H), LC/MS (APCI): m/z=260.0 [M+H]⁺, Rt 3.625 min.

### Intermediate 40: Synthesis of 2-Chloro-4-(3-(difluoromethyl)-5-fluorobenzyl)-6-methylpyridine

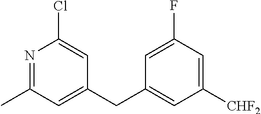 [SMILES: Cc1cc(Cc2cc(F)cc(C(F)F)c2)cc(Cl)n1]

K₂CO₃ (320 mg, 2.31 mmol) was added to a stirred solution of 3-fluoro-5-(difluoromethyl)benzyl chloride (150 mg, 0.771 mmol) and 2-chloro-6-methylpyridine-4-boronic acid pinacol ester (196 mg, 0.771 mmol) in 1,4-dioxane (3.5 mL) and water (1.5 mL). The reaction was degassed with N₂ for 20 min then Pd(dppf)₂Cl₂·CH₂Cl₂ complex (63 mg, 0.077 mmol) was added. The reaction mixture was heated at 100° C. for 2 h. The reaction mixture was partitioned between water (50 mL) and EtOAc (100 mL). The organic layer was separated, and the water layer was extracted with EtOAc (2×50 mL) The combined organic layer was dried over MgSO₄ and concentrated in vacuo. The residue was purified by flash column chromatography eluting with EtOAc:PET (1:9) to afford the title compound as a pale yellow oil (121 mg, 55%). ¹H NMR (400 MHz, Acetone-d6) δ 7.37 (s, 1H), 7.32-7.22 (m, 2H), 7.20-7.15 (m, 2H), 6.90 (t, J=55.9 Hz, 1H), 4.11 (s, 2H), 2.42 (s, 3H), LC/MS (APCI): m/z=286.0 [M+H]⁺, Rt 3.920 min.

### Intermediate 41: Synthesis of 2-chloro-4-(3-(difluoromethyl)-5-fluorobenzyl)-6-methoxypyridine

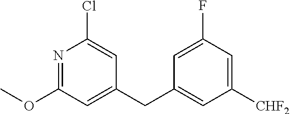 [SMILES: COc1cc(Cc2cc(F)cc(C(F)F)c2)cc(Cl)n1]

K₂CO₃ (213 mg, 1.54 mmol) was added to a stirred solution of methyl 3-fluoro-5-(difluoromethyl)benzyl chloride (100 mg, 0.514 mmol) and 2-chloro-6-methoxypyridine-4-boronic acid pinacol ester (139 mg, 0.514 mmol) in 1,4-dioxane (3.5 mL) and water (1.5 mL). The reaction was degassed with N₂ for 20 min then Pd(dppf)₂Cl₂·CH₂Cl₂ complex (42 mg, 0.051 mmol) was added. The reaction mixture was heated at 100° C. for 3 h. The reaction mixture was partitioned between water (10 mL) and EtOAc (50 mL). The organic layer was separated and the water layer was extracted with EtOAc (2×50 mL) The combined organic layer was dried over MgSO₄ and concentrated in vacuo. The residue was purified by flash silica gel column chromatography eluting with EtOAc:PET (1:9) to afford the title compound as a pale-orange solid (91 mg, 59%). ¹H NMR (400 MHz, Acetone-d6) δ 7.39 (s, 1H), 7.30 (d, J=9.7 Hz, 1H), 7.28-7.22 (m, 1H), 7.06-6.75 (m, 2H), 6.69 (m, 1H), 4.10 (s, 2H), 3.86 (s, 3H), LC/MS (APCI): m/z=302.0 [M+H]⁺, Rt 4.116 min.

### Intermediate 42: Synthesis of 3-(trifluoromethyl)-6,7-dihydroimidazo[1,5-a]pyrazin-8(5H)-one

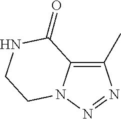

Step 1: To a solution of methyl 5-methyl-1H-1,2,3-triazole-4-carboxylate (150 mg, 1.06 mmol) and N—Z-ethanolamine (311 mg, 1.59 mmol) in anhydrous THF (10 mL) at −40° C. was added Ph₃P (697 mg, 2.66 mmol). DIAD (523 μL, 2.40 mmol) was added dropwise into the solution and the reaction was stirred at −40° C. for 2 h then increased slowly to rt and stirred overnight. Upon completion, silica gel was added and the mixture was concentrated in vacuo. The silica gel mixture was loaded on normal phase column chromatography eluting with EtOAc:PET benzine (1:1) to afford crude product as colorless oil (261 mg) which is a mixture of 3 possible regioisomers: methyl 1-(2-(((benzyloxy)carbonyl)amino)ethyl)-4-methyl-1H-1,2,3-triazole-5-carboxylate, methyl 2-(2-(((benzyloxy)carbonyl)amino)ethyl)-5-methyl-2H-1,2,3-triazole-4-carboxylate, and methyl 1-(2-(((benzyloxy)carbonyl)amino) ethyl)-5-methyl-1H-1,2,3-triazole-4-carboxylate. The mixture was used for the next step without structure elucidation. LC/MS (APCI) analysis observed 2 peaks with the same mass: m/z=319.1 [M+H]⁺, Rt 3.369 and 3.441 min.

Step 2: To a solution of the crude mixture of regioisomers of step 1 (261 mg, 0.82 mmol) in MeOH (10 mL) was added 10% Pd/C (52 mg, 20% wt). The flask was filled with N₂ first and replaced with Hydrogen atmosphere using H₂ balloon. The reaction was vigorously stirred at rt for 72 h. After completion, the mixture was filtered through a pad of Celite®, washed with MeOH, and the solvent was concentrated in vacuo. The residue was purified by flash silica gel column chromatography eluting with 5% MeOH in EtOAc to afford the title compound as a pale-yellow solid (41 mg, 26% after 2 steps). ¹H NMR (400 MHz, DMSO-d6) δ 8.31 (s, 1H), 4.53-4.51 (m, 2H), 3.64-3.59 (m, 2H), 2.41 (s, 3H). LC/MS (APCI): m/z=153.1 [M+H]⁺, Rt 0.554 min.

### Intermediate 43: Synthesis of 5,5-dimethyl-6,7-dihydroimidazo[1,5-a]pyrazin-8(5H)-one

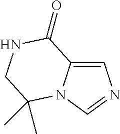

Step 1: To a stirred solution of methyl 1H-imidazole-5-carboxylate (CAS: 17325-26-7) (10.0 g, 79.3 mmol, 1.0 equiv) in DMF (100 mL), was added 2-bromoacetonitrile (CAS: 590-17-0) (11.4 g, 95.2 mmol, 1.2 equiv) and K₂CO₃ (32.8 g, 238 mmol, 3.0 equiv), then the reaction mixture was stirred at room temperature for 2 h. After completion of the reaction as indicated by TLC (5% MeOH in DCM), the reaction mixture was poured into water (500 mL) and extracted with EtOAc (3×100 mL). The combined organic layers were dried over Na₂SO₄ and concentrated under reduced pressure to afford the crude compound. The crude compound was purified by normal phase column chromatography (230-400 silica, gradient 0-4% MeOH in DCM) to afford the two separated regio-isomers: Isomer-01, off-white solid, (3.0 g, 23% yield). ¹H NMR (400 MHz, d₆-DMSO): δ ppm 8.11 (d, J=0.8 Hz, 1H), 7.75 (d, J=1.2 Hz, 1H), 5.49 (s, 2H), 3.84 (s, 3H); LCMS-02 (Method-A): RT 1.29 min, m/z 166.2 [M+H]⁺. Isomer-02, off-white solid, (0.35 g, 3% yield). ¹H NMR (400 MHz, do-DMSO): δ ppm 8.06 (d, J=1.6 Hz, 1H), 7.92 (d, J=1.2 Hz, 1H), 5.39 (s, 2H), 3.76 (s, 3H); LCMS-02 (Method-A): RT 0.86 min, m/z 166.2 [M+H]⁺.

Step 2: To a stirred solution of methyl 1-(cyanomethyl)-1H-imidazole-5-carboxylate (Isomer-01) (3.0 g, 18 mmol, 1.0 equiv) in THF (30 mL), was added methyl iodide (10 g, 73 mmol, 4.0 equiv) at 0° C. and reaction mixture stirred for 10 min at same temperature. The LiHMDS (1.0 M solution in THF) (55 mL, 55 mmol, 3.0 equiv) was added dropwise and the reaction mixture was stirred at room temperature for 5 h. After completion of the reaction as indicated by TLC (5% MeOH in DCM), the reaction mixture was quenched with 1N HCl (60 mL) and extracted with EtOAc (3×30 mL). The organic layers were dried over Na₂SO₄ and concentrated under reduced pressure to afford the crude compound. The crude compound was purified by normal phase column chromatography (230-400 silica, gradient 0-2% MeOH in DCM) to afford methyl 1-(2-cyanopropan-2-yl)-1H-imidazole-5-carboxylate as a light-yellow solid (1.4 g, 40% yield); LCMS-02 (Method-A): RT 1.64 min, m/z 194.3 [M+H]⁺.

Step 3: To a stirred solution of methyl 1-(2-cyanopropan-2-yl)-1H-imidazole-5-carboxylate (1.4 g, 7.246 mmol, 1.0 equiv) in methanol (14.0 mL), was added Pd/C (0.28 g, 20% w/w) and the flask was filled with N₂ first then replace with hydrogen atmosphere using H₂ balloon and the reaction mixture was stirred at room temperature for 12 h. The completion of the reaction was indicated by TLC (10% Methanol in DCM), the reaction mixture was filtered through a celite bed, and the bed was washed with DCM (30 mL). The filtrate was concentrated under reduced pressure to afford the crude compound. The crude was purified by normal phase column chromatography (230-400 silica, 5% MeOH in DCM) to afford 5,5-dimethyl-6,7-dihydroimidazo[1,5-a]pyrazin-8(5H)-one as an off-white solid (0.150 g, 13% yield); ¹H NMR (400 MHz, d₆-DMSO): δ ppm 7.97 (s, 1H), 7.93 (brs, 1H), 7.44 (s, 1H), 1.55 (s, 6H), (2 proton merge in DMSO solvent peak). LCMS-02 (Method-A): RT 0.29 min, m/z 166.3 [M+H]⁺.

Intermediate 44: Synthesis of 1-(hydroxymethyl)-3-methyl-6,7-dihydroimidazo[1,5-a]pyrazin-8(5H)-one

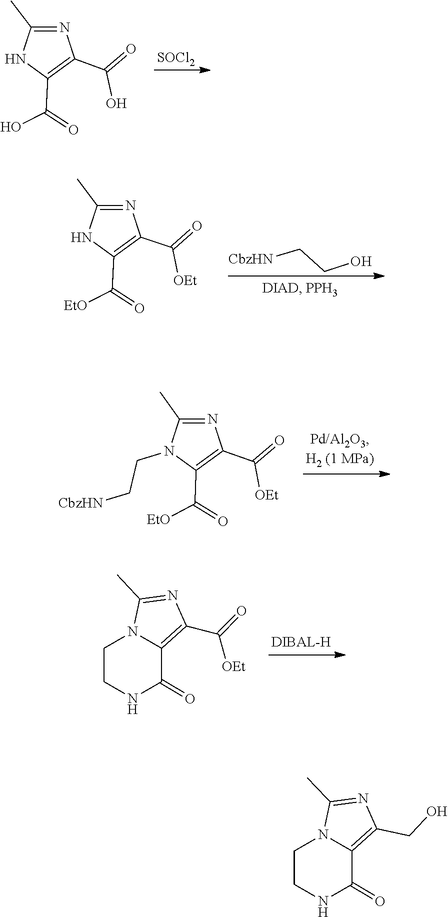

Step A: To a solution of 2-methyl-1H-imidazole-4,5-dicarboxylic acid (2.5 g, 14.70 mmol, 1 eq.) in EtOH (30 mL) was added SOCl₂ (8.74 g, 73.48 mmol, 5.33 mL, 5 eq.) at 0° C. After addition, the mixture was stirred at 20° C. for 0.5 h. The resulting mixture was then heated and stirred at 80° C. for 12 h. Next, the mixture was concentrated and quenched with water (100 mL) and then neutralized with 2 N aq. NaHCO₃ to pH 7. The mixture was extracted with EtOAc (50 mL×5). The combined organic layers were dried over Na₂SO₄, filtered and concentrated to give the residue, which was purified by flash silica gel chromatography (0-100% EtOAc in hexanes) to give diethyl 2-methyl-1H-imidazole-4,5-dicarboxylate (2.65 g, 11.68 mmol, 79.51% yield) as a pale-yellow solid. LCMS [M+H]⁺=227.1.

Step B: To a solution of diethyl 2-methyl-1H-imidazole-4,5-dicarboxylate (2.65 g, 11.71 mmol, 1 eq.), benzyl(2-hydroxyethyl)carbamate (2.97 g, 15.23 mmol, 1.3 eq.), PPh₃ (6.14 g, 23.43 mmol, 2 eq.) in THF (200 mL) was added DIAD (4.74 g, 23.43 mmol, 4.54 mL, 2 eq.) at −60° C. After addition, the mixture was stirred at 20° C. for 12 h under N₂ atmosphere. The mixture was concentrated. The residue was purified by flash silica gel chromatography (0-100% EtOAc in DCM). The residue was adjusted pH to 1 with 2 NHCl (20 mL), the mixture was extracted with EtOAc (100 mL×3), the aqueous phase neutralized with saturated Na₂CO₃ solution to pH 8-9. The mixture was extracted with EtOAc (100 mL×3). The combined organic layers were washed with brine (40 mL), dried over Na₂SO₄, filtered and concentrated to give diethyl 1-(2-(((benzyloxy)carbonyl)amino)ethyl)-2-methyl-1H-imidazole-4,5-dicarboxylate (2.8 g, 6.52 mmol, 55.70% yield) as a white solid: ¹H NMR (400 MHz, DMSO-d₆) δ=7.47 (s, 1H), 7.39-7.31 (m, 5H), 5.00 (s, 2H), 4.25-4.20 (m, 4H), 4.18 (t, J=6.0 Hz, 2H), 3.31-3.28 (m, 2H), 2.31 (s, 3H), 1.25 (dt, J=4.4, 7.2 Hz, 6H).

Step C: The solution diethyl 1-(2-(((benzyloxy)carbonyl)amino)ethyl)-2-methyl-1H-imidazole-4,5-dicarboxylate (2.5 g, 6.20 mmol, 1 eq.) in MeOH (45 mL) was pumped (0.3 mL/min) through a fixed bed (completely packed with granular catalyst 5% Pd/Al₂O₃ (0.2 g) at 50° C. under H₂ (1 MPa, the flow rate of H₂ was 30 mL/min) for 2 h. The mixture was concentrated to give ethyl 3-methyl-8-oxo-5,6,7,8-tetrahydroimidazo[1,5-a]pyrazine-1-carboxylate (1.4 g, crude) as a white solid, which was used into the next step without further purification: ¹H NMR (400 MHz, DMSO-d₆) δ=8.07 (br s, 1H), 4.20 (q, J=7.2 Hz, 2H), 4.09-3.98 (m, 2H), 3.55-3.43 (m, 2H), 2.34 (s, 3H), 1.25 (t, J=7.2 Hz, 3H).

Step D: To a solution of ethyl 3-methyl-8-oxo-5,6,7,8-tetrahydroimidazo[1,5-a]pyrazine-1-carboxylate (400 mg, 1.79 mmol, 1 eq.) in THF (15 mL) and DCM (15 mL) was added DIBAL-H (1 M, 7.17 mL, 4 eq.) at −30° C. The mixture was stirred at 20° C. for 1 h under N₂ atmosphere. The reaction mixture was quenched by addition MeOH (20 mL) and stirred at 80° C. for 1 h under N₂ atmosphere and concentrated under reduced pressure to give a residue, which was purified by flash silica gel chromatography (100% DCM to DCM/MeOH=90/10) to give 1-(hydroxymethyl)-3-methyl-6,7-dihydroimidazo[1,5-a]pyrazin-8(5H)-one (0.28 g, 1.55 mmol, 86.24% yield) as a white solid. LCMS [M+H]⁺=182.1; ¹H NMR (400 MHz, DMSO-d₆) δ=8.04 (br s, 1H), 5.49 (t, J=6.0 Hz, 1H), 4.52 (d, J=5.6 Hz, 2H), 4.02 (t, J=6.0 Hz, 2H), 3.54-3.42 (m, 2H), 2.30 (s, 3H).

### Intermediate 45: Synthesis of 3-(((tert-butyldimethylsilyl)oxy)methyl)-1-methyl-6,7-dihydroimidazo[1,5-a]pyrazin-8(5H)-one

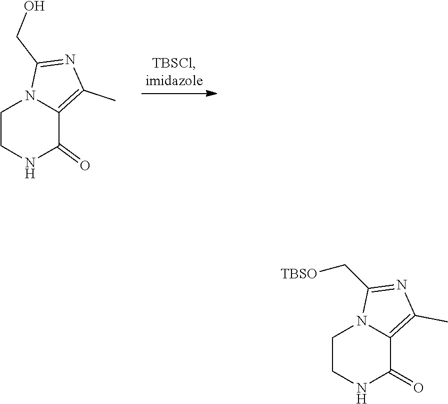 [SMILES: Cc1nc(CO)n2c1C(=O)NCC2 ; Cc1nc(CO[Si](C)(C)C(C)(C)C)n2c1C(=O)NCC2]

Step A: To a solution of 3-(hydroxymethyl)-1-methyl-6,7-dihydroimidazo[1,5-a]pyrazin-8(5H)-one (200 mg, 1.10 mmol, 1 eq.) in THF (4 mL) and DMF (2 mL) was added imidazole (187.86 mg, 2.76 mmol, 2.5 eq.) and tert-Butyldimethylsilyl chloride (249.55 mg, 1.66 mmol, 203.71 μL, 1.5 eq.). The reaction mixture was stirred at 20° C. for 3 h. Then, the reaction mixture was poured into water (20 mL) and extracted with EtOAc (30 mL×3). The combined organic layers were washed with water (30 mL×3), brine (30 mL×3) and dried over Na₂SO₄, filtered and concentrated under reduced pressure. The residue was purified by flash silica gel chromatography (DCM to DCM/MeOH=96/4, TLC: DCM/MeOH=10/1, Rf =0.5) to give 3-(((tert-butyldimethylsilyl)oxy)methyl)-1-methyl-6,7-dihydroimidazo[1,5-a]pyrazin-8(5H)-one (130 mg, 440.00 μmol, 39.86% yield) as a white solid. LCMS [M+H]⁺=296.2; ¹H NMR (400 MHz, CDCl₃) δ=5.90 (s, 1H), 4.79 (s, 2H), 4.29-4.15 (m, 2H), 3.69-3.59 (m, 2H), 2.51 (s, 3H), 0.88 (s, 9H), 0.07 (s, 6H).

### Intermediate 46: Synthesis of 1-(2-((tert-butyldimethylsilyl)oxy)ethyl)-3-methyl-1,5,6,7-tetrahydro-4H-pyrazolo[4,3-c]pyridin-4-one

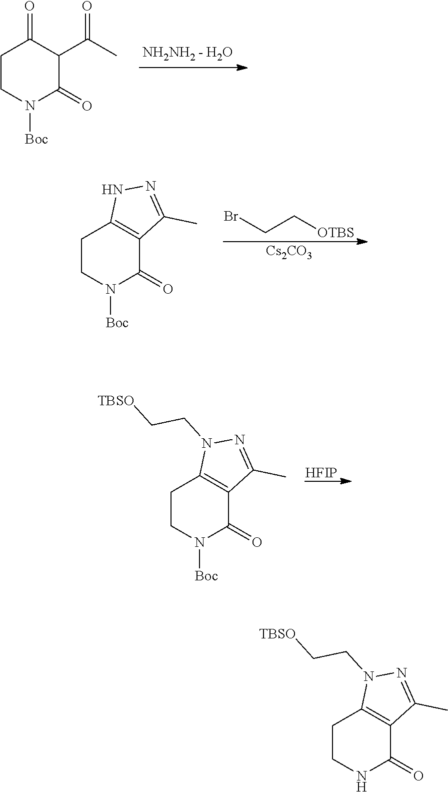

Step A: To a solution of tert-butyl 3-acetyl-2,4-dioxopiperidine-1-carboxylate (3 g, 11.75 mmol, 1 eq.) in THF (20 mL) was dropwise added NH₂NH₂—H₂O (1.04 g, 17.63 mmol, 1.01 mL, 1.5 eq.) at 20° C. under N₂, then the mixture was stirred at 20° C. for 16 h. The mixture was diluted with water (30 mL), the resulting mixture was extracted with EtOAc (40 mL×2). The combined organic layer was dried over anhydrous sodium sulfate and concentrated under reduced pressure. The crude product was purified by flash silica gel chromatography (100% hexanes to hexanes/EtOAc=80/20, R_{f}=0.30) to give tert-butyl 3-methyl-4-oxo-1,4,6,7-tetrahydro-5H-pyrazolo[4,3-c]pyridine-5-carboxylate (2.5 g, 8.95 mmol, 76.19% yield) as colorless oil. LCMS [M+H]⁺=252.1; ¹H NMR (400 MHz, CDCl₃) δ=4.05 (t, J=6.0 Hz, 2H), 2.91 (t, J=6.4 Hz, 2H), 2.57 (s, 3H), 1.56 (s, 9H).

Step B: To a solution of tert-butyl 3-methyl-4-oxo-1,4,6,7-tetrahydro-5H-pyrazolo[4,3-c]pyridine-5-carboxylate (2.5 g, 9.95 mmol, 1 eq.) and Cs₂CO₃ (6.48 g, 19.90 mmol, 2 eq.) in ACN (30 mL) was dropwise added (2-bromoethoxy) (tert-butyl)dimethylsilane (3.09 g, 12.93 mmol, 1.3 eq.) at 20° C. Then the mixture was heated and stirred at 50° C. for 16 h. The mixture was diluted with water (30 mL), the resulting mixture was extracted with EtOAc (40 mL×2). The combined organic layer was dried over anhydrous sodium sulfate and concentrated under reduced pressure. The crude was purified by prep-HPLC (column: 41-WePure Biotech XP tC18 150×40 mm, 7 μm; mobile phase: [H₂O (10 mM NH₄HCO₃)-ACN]; gradient: 55%-85% ACN over 11.0 min; Flow rate: 50.00 mL/min) to give tert-butyl 1-(2-((tert-butyldimethylsilyl)oxy)ethyl)-3-methyl-4-oxo-1,4,6,7-tetrahydro-5H-pyrazolo[4,3-c]pyridine-5-carboxylate (1.1 g, 2.55 mmol, 25.64% yield,) as colorless oil. LCMS [M+H]⁺=410.3; ¹H NMR (400 MHz, CDCl₃) δ=4.06-4.03 (m, 4H), 3.93 (t, J=4.8 Hz, 2H), 2.90 (t, J=6.4 Hz, 2H), 2.47 (s, 3H), 1.56 (s, 9H), 0.81 (s, 9H), −0.08 (s, 6H).

Step C: A solution of tert-butyl 1-(2-((tert-butyldimethylsilyl)oxy)ethyl)-3-methyl-4-oxo-1,4,6,7-tetrahydro-5H-pyrazolo[4,3-c]pyridine-5-carboxylate (200 mg, 488.29 μmol, 1 eq.) in HFIP (1,1,1,3,3,3-hexafluoro-2-propanol, 10 mL) was stirred at 70° C. for 1 h. The mixture was diluted with water (20 mL), the resulting mixture was extracted by DCM (20 mL×2). The combined organic layer was dried over anhydrous sodium sulfate and concentrated under reduced pressure to give 1-(2-((tert-butyldimethylsilyl)oxy)ethyl)-3-methyl-1,5,6,7-tetrahydro-4H-pyrazolo[4,3-c]pyridin-4-one (150 mg, crude) as a white solid, which will be used for the next step without further purification. LCMS [M+H]⁺=310.0; ¹H NMR (400 MHz, CDCl₃) δ=5.22 (s, 1H), 4.07 (t, J=4.4 Hz, 2H), 3.95 (t, J=4.8 Hz, 2H), 3.57-3.54 (m, 2H), 2.90 (t, J=6.8 Hz, 2H), 2.48 (s, 3H), 0.81 (s, 9H), −0.08 (s, 6H).

### Intermediate 47: Synthesis of 1-methyl-6,7-dihydroimidazo[1,5-a]pyrazin-8(5H)-one

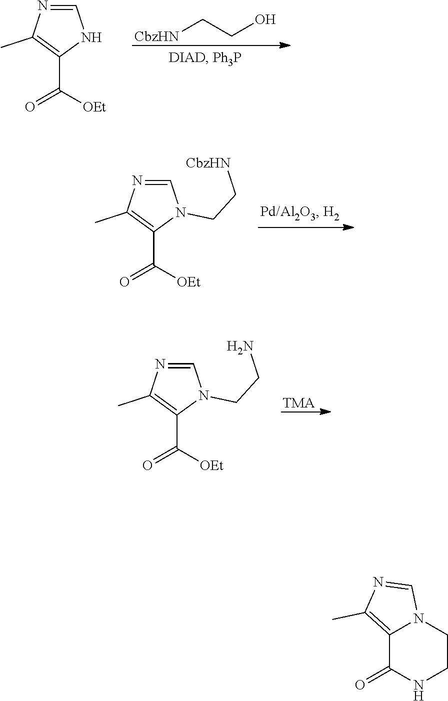

Step A: To a solution of ethyl 4-methyl-1H-imidazole-5-carboxylate (5 g, 32.43 mmol, 1 eq.) and benzyl(2-hydroxyethyl)carbamate (8.23 g, 42.16 mmol, 1.3 eq.) in THF (1000 mL) was added PPh₃ (17.01 g, 64.87 mmol, 2 eq.) and DIAD (13.12 g, 64.87 mmol, 12.58 mL, 2 eq.) at −60° C. under N₂. The mixture was stirred at 20° C. for 16 h under N₂. The reaction mixture was concentrated under reduced pressure to give a residue, which was purified by flash silica gel chromatography (hexane to hexane/EtOAc=0/100, TLC:hexane/EtOAc=0/1, R_{f}=0.30) to yield ethyl 1-(2-(((benzyloxy)carbonyl)amino)ethyl)-4-methyl-1H-imidazole-5-carboxylate (7 g, 20.07 mmol, 61.88% yield,) as a white solid: ¹H NMR (400 MHz, CDCl₃) δ=7.63 (s, 1H), 7.38-7.27 (m, 6H), 4.98 (s, 2H), 4.29-4.20 (m, 4H), 3.32-3.3 (m, 2H), 2.35 (s, 3H), 1.29 (t, J=7.2 Hz, 3H).

Step B: A solution of ethyl 1-(2-(((benzyloxy)carbonyl)amino)ethyl)-4-methyl-1H-imidazole-5-carboxylate (7 g in 140 mL MeOH) was pumped (0.3 mL/min) through a fixed bed (completely packed with granular catalyst 5% Pd/Al₂O₃) at 70° C. under H₂ (1 MPa, the flow rate of H₂ was 30 mL/min). Then the reaction mixture was collected from the reactor output. The reaction mixture was concentrated under reduced pressure to give ethyl 1-(2-aminoethyl)-4-methyl-1H-imidazole-5-carboxylate (4.15 g, crude) as a white solid, which was used directly in the next step without further purification.

Step C: To a solution of ethyl 1-(2-aminoethyl)-4-methyl-1H-imidazole-5-carboxylate (4.15 g, 19.99 mmol) in toluene (300 mL) was added TMA (2 M in toluene, 20 mL, 1 eq.). The mixture was stirred at 80° C. for 1 h. The reaction mixture was quenched by addition H₂O (200 mL) and extracted with EtOAc (300 mL×3). The combined organic layers were washed with brine (300 mL), dried over Na₂SO₄, filtered and concentrated under reduced pressure to give a residue. The residue was purified by flash silica gel chromatography (DCM to DCM/MeOH=94/6, TLC: DCM/MeOH=10/1, R_{f}=0.40) to yield 1-methyl-6,7-dihydroimidazo[1,5-a]pyrazin-8(5H)-one (1.75 g, 11.00 mmol, 55.02% yield) as a white solid: ¹H NMR (400 MHz, DMSO-d₆) δ=7.73 (br s, 1H), 7.66 (s, 1H), 4.13-4.08 (m, 2H), 3.47-3.41 (m, 2H), 2.32 (s, 3H).

### Intermediate 48: Synthesis of 3-(2-hydroxyethyl)-1-methyl-6,7-dihydroimidazo[1,5-a]pyrazin-8(5H)-one

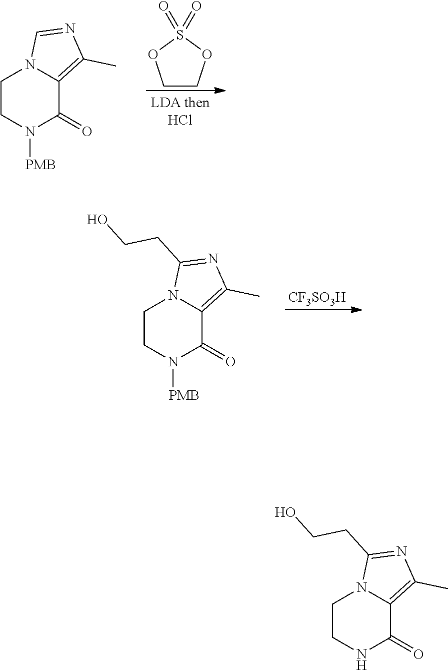

Step A: A solution of 7-(4-methoxybenzyl)-1-methyl-6,7-dihydroimidazo[1,5-a]pyrazin-8(5H)-one (300 mg, 1.11 mmol, 1 eq.) in THF (12 mL) was prepared (Solution 1). A separate solution of LDA (2 M, 884.58 μL, 1.6 eq.) in THF was prepared (Solution 2). A solution of 1,2-ethylene sulfate (274.48 mg, 2.21 mmol, 2 eq.) in THF (7.5 mL) was also prepared (Solution 3). Solution 1 was pumped by Pump 1 {18.891 mL/min} to flow reactor 1 {PFA, Coils reactor, 1.590 ( 1/16″) mm, 0.54 mL, −20.0° C.}. Solution 2 was pumped by Pump 2 {2.709 mL/min} to flow reactor 1 {PFA, Coils reactor, 1.590 ( 1/16″) mm, 0.54 mL, −20.0° C.}. Solution 3 was pumped by Pump 3 {12.004 mL/min} to flow reactor 2 {PFA, Coils reactor, 1.590 ( 1/16″) mm, 0.54 mL, −20.0° C.}. The residence time of flow reactor 1 was 0.025 min. The residence time of flow reactor 2 was 0.016 min. The mixture was collected with a bottle and quenched by 12 M HCl (6 mL) at 20° C. for 3 h. Then this mixture was treated with aqueous NaHCO₃ (30 mL) at 0° C., and extracted with DCM (30 mL×3). The combined organic layers were dried over Na₂SO₄, filtered and concentrated under reduced pressure to give a residue. The residue was purified by flash silica gel chromatography (DCM to DCM/MeOH=91/9) to yield 3-(2-hydroxyethyl)-7-(4-methoxybenzyl)-1-methyl-6,7-dihydroimidazo[1,5-a]pyrazin-8(5H)-one (140 mg, 399.54 μmol, 36.13% yield) as a yellow solid. LCMS [M+H]⁺=316.2; ¹H NMR (400 MHz, DMSO-d₆) δ=7.23 (d, J=8.4 Hz, 2H), 6.93-6.87 (m, 2H), 4.76 (t, J=5.2 Hz, 1H), 4.56 (s, 2H), 4.10-4.01 (m, 2H), 3.73 (s, 3H), 3.70-3.62 (m, 2H), 3.53-3.46 (m, 2H), 2.73 (t, J=6.4 Hz, 2H), 2.34 (s, 3H).

Step B: To a solution of 3-(2-hydroxyethyl)-7-(4-methoxybenzyl)-1-methyl-6,7-dihydroimidazo[1,5-a]pyrazin-8(5H)-one (170 mg, 539.06 μmol, 1 eq.) in toluene (10 mL) was added CF₃SO₃H (809.00 mg, 5.39 mmol, 477.00 μL, 10 eq.) at 0° C. The mixture was stirred at 20° C. for 16 h. The reaction mixture was quenched by the addition of aq. NaHCO₃ (10 mL) at 0° C., and then extracted with H₂O (5 mL×2). The combined aqueous layers were filtered. The residue was purified by reversed-phase silica gel column (H₂O to H₂O/MeCN=92/8) to yield 3-(2-hydroxyethyl)-1-methyl-6,7-dihydroimidazo[1,5-a]pyrazin-8(5H)-one (85 mg, 391.87 μmol, 72.70% yield) as a white solid: ¹H NMR (400 MHz, DMSO-d₆) δ=7.72-7.59 (m, 1H), 4.78 (br s, 1H), 4.01 (t, J=5.6 Hz, 2H), 3.67 (br t, J=6.4 Hz, 2H), 3.42 (br t, J=6.4 Hz, 2H), 2.75 (t, J=6.4 Hz, 2H), 2.29 (s, 3H).

### Intermediate 49: Synthesis of 1-bromo-3-(hydroxymethyl)-6,7-dihydroimidazo[1,5-a]pyrazin-8(5H)-one

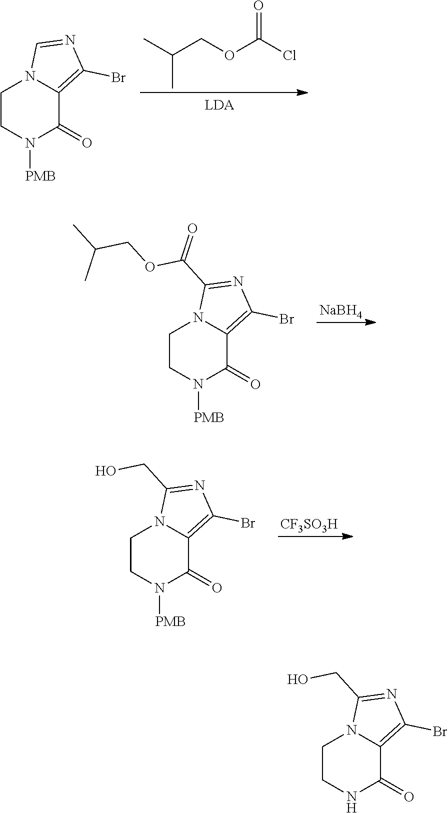

Step A: A solution of 1-bromo-7-(4-methoxybenzyl)-6,7-dihydroimidazo[1,5-a]pyrazin-8(5H)-one (1 g, 2.97 mmol, 1 eq.) in THF (50 mL) was prepared (Solution 1). A solution of LDA (2 M, 2.68 mL, 1.8 eq.) in THF was prepared (Solution 2). A solution of isobutyl chloroformate (1.22 g, 8.92 mmol, 1.17 mL, 3 eq.) in THF (25 mL) was prepared (Solution 3). Solution 1 was pumped by Pump 1 {19.552 mL/min} to flow reactor 1 {PFA, Coils reactor, 1.590 ( 1/16″) mm, 0.54 mL, −30.0° C.}. Solution 2 was pumped by Pump 2 {2.048 mL/min} to flow reactor 1 {PFA, Coils reactor, 1.590 ( 1/16″) mm, 0.54 mL, −30.0° C.}. Solution 3 was pumped by Pump 3 {10.085 mL/min} to flow reactor 2 {PFA, Coils reactor, 1.590 ( 1/16″) mm, 0.54 mL, −30.0° C.}. The residence time of flow reactor 1 was 0.025 min. The residence time of flow reactor 2 was 0.017 min. The mixture was collected with a bottle and quenched by 30 mL aq. NH₄Cl at 0° C., then extracted with EtOAc (30 mL×3). The combined organic layers were dried over Na₂SO₄, filtered and concentrated under reduced pressure to give a residue. The residue was purified by flash silica gel chromatography (hexanes to hexanes/EtOAc=67/33) to yield isobutyl 1-bromo-7-(4-methoxybenzyl)-8-oxo-5,6,7,8-tetrahydroimidazo[1,5-a]pyrazine-3-carboxylate (385 mg, 794.18 μmol, 26.70% yield) as a yellow solid. LCMS [M+H]⁺=438.0; ¹H NMR (400 MHz, DMSO-d₆) δ=7.26 (d, J=8.4 Hz, 2H), 6.91 (d, J=8.0 Hz, 2H), 4.64-4.51 (m, 4H), 4.12-4.03 (m, 2H), 3.73 (s, 3H), 3.63 (br t, J=5.6 Hz, 2H), 2.03-1.97 (m, 1H), 0.94 (d, J=6.0 Hz, 6H).

Step B: To a solution of isobutyl 1-bromo-7-(4-methoxybenzyl)-8-oxo-5,6,7,8-tetrahydroimidazo[1,5-a]pyrazine-3-carboxylate (385 mg, 882.42 μmol, 1 eq.) in MeOH (15 mL) was added NaBH₄ (180 mg, 4.76 mmol, 5.39 eq.) at 0° C. After addition, the mixture was stirred at 20° C. for 6 h. The reaction mixture was quenched by the addition of aq. NH₄Cl (10 mL) at 0° C., and then diluted with H₂O 50 mL, extracted with EtOAc (50 mL×3). The combined organic layers were filtered. The residue was purified by flash silica gel chromatography (DCM to DCM/MeOH=97/3) to yield 1-bromo-3-(hydroxymethyl)-7-(4-methoxybenzyl)-6,7-dihydroimidazo[1,5-a]pyrazin-8(5H)-one (310 mg, 804.19 μmol, 91.13% yield) as a white solid. LCMS [M+H]⁺=368.0; ¹H NMR (400 MHz, DMSO-d₆) δ=7.24 (d, J=8.8 Hz, 2H), 6.91 (d, J=8.8 Hz, 2H), 5.50 (t, J=5.6 Hz, 1H), 4.56 (s, 2H), 4.47 (d, J=6.0 Hz, 2H), 4.24-4.16 (m, 2H), 3.73 (s, 3H), 3.61-3.53 (m, 2H).

Step C: To a solution of 1-bromo-3-(hydroxymethyl)-7-(4-methoxybenzyl)-6,7-dihydroimidazo[1,5-a]pyrazin-8(5H)-one (310 mg, 846.51 μmol, 1 eq.) in toluene (16 mL) 5 was added CF₃SO₃H (1.27 g, 8.47 mmol, 749.07 μL, 10 eq.) at 0° C. After addition, the mixture was stirred at 20° C. for 16 h. The reaction mixture was quenched by the addition of aq. NaHCO₃ (10 mL) at 0° C., and then extracted with H₂O (10 mL×2). The combined aqueous layers were filtered and purified by reversed-phase silica gel column (H₂O to H₂O/MeCN=93/7) to yield 1-bromo-3-(hydroxymethyl)-6,7-dihydroimidazo[1,5-a]pyrazin-8(5H)-one (125 mg, 457.20 μmol, 54.01% yield) as a white solid: ¹H NMR (400 MHz, 10 DMSO-d₆) δ=8.00 (br s, 1H), 5.85-5.11 (m, 1H), 4.49 (s, 2H), 4.25-4.10 (m, 2H), 3.50-3.47 (m, 2H).

### Intermediate 50: Synthesis of 3-(((tert-butyldimethylsilyl)oxy)methyl)-1-ethyl-6,7-dihydroimidazo[1,5-a]pyrazin-8(5H)-one

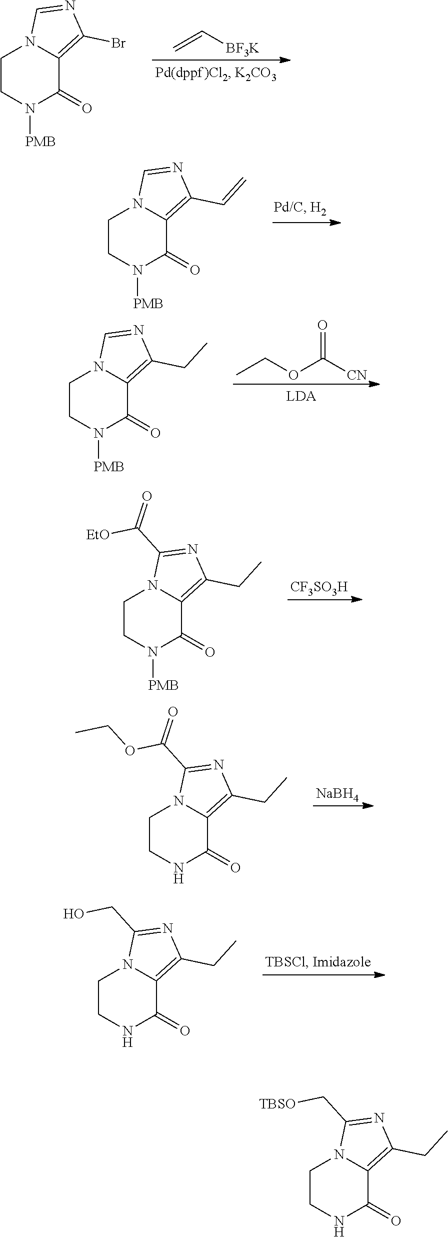

Step A: To a solution of 1-bromo-7-(4-methoxybenzyl)-6,7-dihydroimidazo[1,5-a]pyrazin-8(5H)-one (1.9 g, 5.65 mmol, 1 eq.) and potassium vinyltrifluoroborate (1.51 g, 11.30 mmol, 2 eq.) in dioxane (10 mL) and H₂O (3 mL) was added Pd(dppf) C12 (413.54 mg, 565.17 μmol, 0.1 eq.) and K₂CO₃ (2.34 g, 16.96 mmol, 3 eq.), the mixture was stirred at 80° C. for 12 h under N₂ atmosphere. Then the reaction mixture was cooled, filtered, and concentrated under vacuum. The residue was purified by flash silica gel chromatography (hexane to hexane/EtOAc=70/30, TLC: hexane/EtOAc=5/1, R_{f}=0.5) to yield 7-(4-methoxybenzyl)-1-vinyl-6,7-dihydroimidazo[1,5-a]pyrazin-8(5H)-one (800 mg, 2.82 mmol, 49.96% yield) as a red solid: ¹H NMR (400 MHz, DMSO-d₆) δ=7.74 (s, 1H), 7.30-7.18 (m, 3H), 6.90 (d, J=8.4 Hz, 2H), 6.10-5.91 (m, 1H), 5.32-5.18 (m, 1H), 4.58 (s, 2H), 4.25-4.12 (m, 2H), 3.73 (s, 3H), 3.55 (t, J=6.0 Hz, 2H).

Step B: To a solution of 7-(4-methoxybenzyl)-1-vinyl-6,7-dihydroimidazo[1,5-a]pyrazin-8(5H)-one (1.3 g, 4.59 mmol, 1 eq.) in MeOH (50 mL) was added Pd/C (3.25 g, 3.05 mmol, 10% purity, 0.67 eq.) under N₂ atmosphere. The suspension was degassed and purged with hydrogen three times. The mixture was stirred under hydrogen (15 Psi) at 15° C. for 6 h. The reaction mixture was filtered and concentrated under vacuum. The residue was purified by flash silica gel chromatography (EtOAc to EtOAc/MeOH=95/5, TLC: EtOAc/MeOH=20/1, R_{f}=0.2) to yield 1-ethyl-7-(4-methoxybenzyl)-6,7-dihydroimidazo[1,5-a]pyrazin-8(5H)-one (1.1 g, 3.86 mmol, 84.02% yield) as a colorless oil: ¹H NMR (400 MHz, DMSO-d₆) δ=7.64 (s, 1H), 7.23 (d, J=8.4 Hz, 2H), 6.90 (d, J=8.8 Hz, 2H), 4.56 (s, 2H), 4.20-4.10 (m, 2H), 3.73 (s, 3H), 3.56-3.46 (m, 2H), 2.81 (q, J=7.6 Hz, 2H), 1.19-1.09 (m, 3H).

Step C: A solution of 1-ethyl-7-(4-methoxybenzyl)-6,7-dihydroimidazo[1,5-a]pyrazin-8(5H)-one (1.0 eq, 0.95 g) in THF (47.5 ml) was prepared (Solution 1). A solution of LDA (2M, 1.4 eq.) in THF was prepared (Solution 2). A solution of ethyl cyanoformate (3.0 eq, 0.99 g) in THF (23.75 ml) was prepared (Solution 3). Solution 1 was pumped by Pump 1 {16.424 mL/min} to flow reactor 1 {Coils reactor, 1.590 ( 1/16″) mm, 0.6 mL, −25.0° C.}. Solution 2 was pumped by Pump 2 {1.576 mL/min} to flow reactor 1 {PFA, Coils reactor, 1.590 ( 1/16″) mm, 0.6 mL, −25.0° C.}. Solution 3 was pumped by Pump 3 {8.408 mL/min} to flow reactor 2 {PFA, Coils reactor, 1.590 ( 1/16″) mm, 0.9 mL, −25.0° C.}. The residence time of flow reactor 1 was 0.033 min. The residence time of flow reactor 2 was 0.034 min. The mixture was collected with a bottle and quenched by aq. NH₄Cl (30 mL) at 0° C. Then the mixture was extracted with EtOAc (5 mL×3). The combined organic layers were dried over Na₂SO₄, filtered and concentrated under reduced pressure to give a residue. The residue was purified by flash silica gel chromatography (hexane to hexane/EtOAc=70/30, TLC: hexane/EtOAc=1/1, R_{f}=0.6) to yield ethyl 1-ethyl-7-(4-methoxybenzyl)-8-oxo-5,6,7,8-tetrahydroimidazo[1,5-a]pyrazine-3-carboxylate (300 mg, 839.39 μmol, 26.61% yield) as a red oil: ¹H NMR (400 MHz, DMSO-d₆) δ=7.25 (d, J=8.8 Hz, 2H), 6.91 (d, J=8.8 Hz, 2H), 4.58 (s, 2H), 4.55-4.48 (m, 2H), 4.30 (q, J=6.8 Hz, 2H), 3.73 (s, 3H), 3.60-3.54 (m, 2H), 2.86 (q, J=7.6 Hz, 2H), 1.30 (t, J=7.2 Hz, 3H), 1.17 (t, J=7.2 Hz, 3H).

Step D: To a solution of ethyl 1-ethyl-7-(4-methoxybenzyl)-8-oxo-5,6,7,8-tetrahydroimidazo[1,5-a]pyrazine-3-carboxylate (600 mg, 1.68 mmol, 1 eq.) in toluene (20 mL) was added CF₃SO₃H (2.52 g, 16.79 mmol, 1.49 mL, 10 eq.) at 0° C., the mixture was stirred at 20° C. for 12 h. The reaction mixture was adjusted to PH ~6 with aq. Na₂CO₃, then extracted with EtOAc (10 mL×3). The combined organic layers were dried over Na₂SO₄, filtered and concentrated under reduced pressure. The residue was purified by flash silica gel chromatography (DCM to DCM/MeOH=95/5, TLC: DCM/MeOH=10/1, R_{f}=0.4) to yield ethyl 1-ethyl-8-oxo-5,6,7,8-tetrahydroimidazo[1,5-a]pyrazine-3-carboxylate (250 mg, 1.05 mmol, 62.77% yield) as a white solid: ¹H NMR (400 MHz, CDCl₃) δ=5.93 (br s, 1H), 4.78-4.64 (m, 2H), 4.47 (q, J=7.2 Hz, 2H), 3.74-3.64 (m, 2H), 3.01 (q, J=7.6 Hz, 2H), 1.45 (t, J=7.2 Hz, 3H), 1.29 (t, J=7.6 Hz, 3H).

Step E: To a solution of ethyl 1-ethyl-8-oxo-5,6,7,8-tetrahydroimidazo[1,5-a]pyrazine-3-carboxylate (250 mg, 1.05 mmol, 1 eq.) in MeOH (10 mL) was added NaBH₄ (119.59 mg, 3.16 mmol, 3 eq.) at 0° C., the mixture was stirred at 15° C. for 12 h under N₂ atmosphere. The reaction mixture was quenched by addition aq. NH₄Cl (10 mL) at 0° C., then extracted with EtOAc (10 mL×3). The combined organic layers were dried over Na₂SO₄, filtered and concentrated under reduced pressure. The residue was purified by reversed-phase silica gel column (water to water/ACN=10/90) to yield 1-ethyl-3-(hydroxymethyl)-6,7-dihydroimidazo[1,5-a]pyrazin-8(5H)-one (170 mg, 870.82 μmol, 82.64% yield) as a white solid. ¹H NMR (400 MHz, DMSO-d₆) δ=7.72 (br s, 1H), 5.59-5.24 (m, 1H), 4.47 (s, 2H), 4.17-4.00 (m, 3H), 3.46-3.41 (m, 2H), 2.75 (q, J=7.6 Hz, 2H), 1.12 (t, J=7.6 Hz, 3H).

Step F: To a solution of 1-ethyl-3-(hydroxymethyl)-6,7-dihydroimidazo[1,5-a]pyrazin-8(5H)-one (150 mg, 768.37 μmol, 1 eq.) in DMF (20 mL) and DCM (10 mL) was added TBSCI (289.53 mg, 1.92 mmol, 236.35 μL, 2.5 eq.) and imidazole (156.93 mg, 2.31 mmol, 3 eq.), the mixture was stirred at 20° C. for 12 h. The reaction mixture was diluted with DCM (200 mL), washed by water (100 mL) and brine (50 mL). The organic layer was dried over Na₂SO₄ and concentrated under vacuum. The residue was purified by flash silica gel chromatography (DCM to DCM/MeOH=95/5, TLC: DCM/MeOH=10/1, R_{f}=0.6) to yield 3-(((tert-butyldimethylsilyl)oxy)methyl)-1-ethyl-6,7-dihydroimidazo[1,5-a]pyrazin-8(5H)-one (170 mg, 549.31 μmol, 71.49% yield) as a white solid: ¹H NMR (400 MHz, CDCl₃) δ=5.70 (br s, 1H), 4.83 (s, 2H), 4.34-4.17 (m, 2H), 3.74-3.54 (m, 2H), 2.95 (q, J=7.2 Hz, 2H), 1.27 (t, J=7.2 Hz, 3H), 0.89 (s, 9H), 0.08 (s, 6H).

### Intermediate 51: Synthesis of 2-bromo-4-((2-fluoropyridin-4-yl)oxy)pyridine

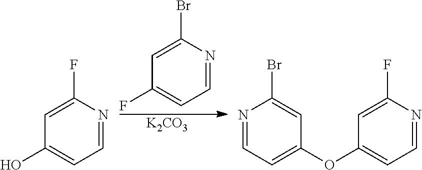

Step A: To a solution of 2-fluoropyridin-4-ol (0.8 g, 7.07 mmol, 1 eq.) in DMF (8 mL) was added K₂CO₃ (1.47 g, 10.61 mmol, 1.5 eq.) and 2-bromo-4-fluoropyridine (1.24 g, 7.07 mmol, 1 eq.). The mixture was stirred at 80° C. for 14 h. The reaction mixture was diluted with H₂O (20 mL) and extracted with EtOAc (45 mL×3), The combined organic layers were dried over Na₂SO₄, filtered and concentrated under reduced pressure to give a residue. The residue was purified by flash silica gel chromatography (hexanes to hexanes/EtOAc=20/3, TLC: hexanes/EtOAc=3/1, R_{f}=0.4) to yield 2-bromo-4-((2-fluoropyridin-4-yl)oxy)pyridine (1.6 g, 5.95 mmol, 84.06% yield) as a white solid: ¹H NMR (400 MHz, CDCl₃) δ=8.40 (d, J=5.6 Hz, 1H), 8.25 (d, J=6.0 Hz, 1H), 7.22 (d, J=2.4 Hz, 1H), 7.04-6.95 (m, 1H), 6.94-6.86 (m, 1H), 6.61 (s, 1H).

### Intermediate 52: Synthesis of 2-bromo-4-(3-cyclopropyl-5-fluorobenzyl)pyridine

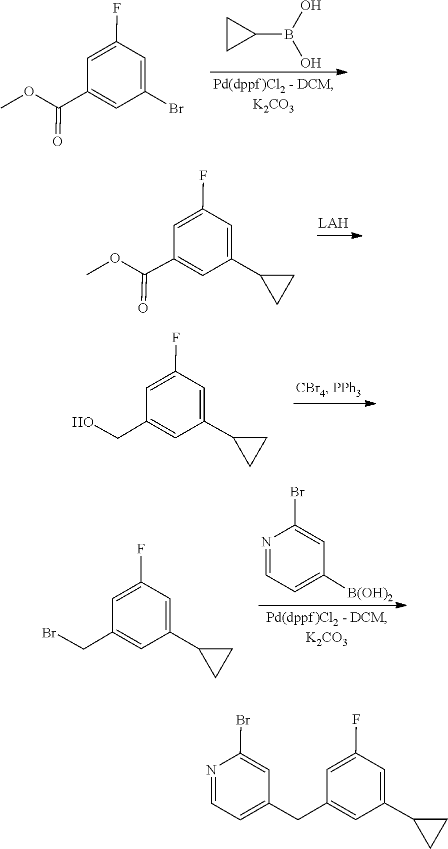

Step A: To a solution of cyclopropylboronic acid (2.21 g, 25.75 mmol, 1.2 eq.) and methyl 3-bromo-5-fluorobenzoate (5 g, 21.46 mmol, 1 eq.) in dioxane (100 mL) and H₂O (20 mL) were added Pd(dppf)Cl₂-DCM (1.75 g, 2.15 mmol, 0.1 eq.) and K₂CO₃ (11.86 g, 85.82 mmol, 4 eq.) under N₂ atmosphere. The resulting mixture was stirred at 90° C. for 5 h under N₂ atmosphere. Then, the reaction mixture was cooled to room temperature, poured into water (200 mL), extracted with EtOAc (200 mL×3). The organic phase was washed with brine (500 mL), dried over anhydrous Na₂SO₄, and filtered. The filtrate was concentrated under reduced pressure, which was purified by flash silica gel chromatography (hexane to hexane/EtOAc=90/10) to give methyl 3-cyclopropyl-5-fluorobenzoate (3.73 g, 18.25 mmol, 85.04% yield) as yellow oil: ¹H NMR (400 MHz, CDCl₃) δ=7.56-7.53 (m, 1H), 7.50-7.45 (m, 1H), 6.95 (td, J=2.0, 9.6 Hz, 1H), 3.91 (s, 3H), 1.98-1.90 (m, 1H), 1.10-0.93 (m, 2H), 0.80-0.69 (m, 2H).

Step B: Lithium aluminum hydride (2.5M in THF) (2.5 M, 7.63 mL, 1.5 eq.) was added dropwise to a stirred solution of methyl 3-cyclopropyl-5-fluorobenzoate (2.6 g, 12.72 mmol, 1 eq.) in THF (30 mL) at 0° C. under N₂ atmosphere. The reaction mixture was stirred at 25° C. for 1.5 h. Na₂SO₄ dodecahydrate (10 g) was added slowly into the reaction mixture at 0° C. under N₂ atmosphere and maintained the temperature below 5° C. The mixture was stirred at 5° C. for 30 min. The reaction mixture was filtered and the filter cake was washed with THF (10 mL×2). The filtrate was collected and concentrated under vacuum to give (3-cyclopropyl-5-fluorophenyl) methanol (2.07 g, crude) as yellow oil, which was used for the next step directly without further purification: ¹H NMR (400 MHz, CDCl₃) δ=6.88-6.82 (m, 2H), 6.70-6.63 (m, 1H), 4.64 (s, 2H), 1.95-1.85 (m, 2H), 1.06-0.93 (m, 2H), 0.76-0.65 (m, 2H).

Step C: To a solution of (3-cyclopropyl-5-fluorophenyl) methanol (2.07 g, 12.46 mmol, 1 eq.) in DCM (50 mL) were added CBr₄ (6.20 g, 18.68 mmol, 1.5 eq.) and PPh₃ (3.92 g, 14.95 mmol, 1.2 eq.) at 0° C. under N₂ atmosphere. The mixture was stirred at 20° C. for 2 h under N₂ atmosphere. The reaction mixture was concentrated under reduced pressure to give a residue, which was triturated with Hexane/EtOAc (100 mL, v/v=3:1) and stirred at 20° C. for 2 h. The mixture was filtered. The filter cake was washed with Hexane/EtOAc (2×50 mL, v/v=3:1). The filtrates were collected and then concentrated under vacuum. The crude product was purified by flash silica gel chromatography (hexane to hexane/EtOAc=98/2) to give 1-(bromomethyl)-3-cyclopropyl-5-fluorobenzene (2.85 g, 12.44 mmol, 100.00% yield) as yellow oil: ¹H NMR (400 MHz, CDCl₃) δ=6.93-6.86 (m, 2H), 6.70-6.64 (m, 1H), 4.42 (s, 2H), 1.94-1.85 (m, 1H), 1.05-0.98 (m, 2H), 0.75-0.68 (m, 2H).

Step D: To a solution of 1-(bromomethyl)-3-cyclopropyl-5-fluorobenzene (2.85 g, 12.44 mmol, 1.14 eq.) and (2-bromopyridin-4-yl) boronic acid (2.2 g, 10.90 mmol, 1 eq.) in dioxane (50 mL) and H₂O (10 mL) were added Pd(dppf)Cl₂-DCM (890.23 mg, 1.09 mmol, 0.1 eq.) and K₂CO₃ (4.52 g, 32.70 mmol, 3 eq.) under N₂ atmosphere. The resulting mixture was stirred at 30° C. for 12 h under N₂ atmosphere. The reaction was cooled to room temperature, poured into water, and then extracted with EtOAc (100 mL×3). The organic phase was washed with brine (100 mL), dried over anhydrous Na₂SO₄, and filtered. The filtrate was concentrated under reduced pressure. The residue was purified by silica gel chromatography (hexane to hexane/EtOAc=50/50) to give 2-bromo-4-(3-cyclopropyl-5-fluorobenzyl)pyridine (600 mg, 1.96 mmol, 17.98% yield) as colorless oil. LCMS [M+H]⁺=305.9; ¹H NMR (400 MHz, CDCl₃) δ=8.27 (d, J=5.2 Hz, 1H), 7.31-7.28 (m, 1H), 7.07-7.04 (m, 1H), 6.71-6.68 (m, 1H), 6.65-6.58 (m, 2H), 3.88 (s, 2H), 1.91-1.82 (m, 1H), 1.04-0.96 (m, 2H), 0.71-0.64 (m, 2H).

### Intermediate 53: Synthesis of 2-bromo-4-(3-cyclopropyl-5-fluorophenoxy)pyridine

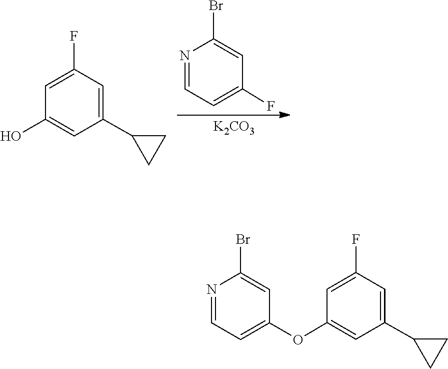

Step A: To a solution of 2-bromo-4-fluoropyridine (867.41 mg, 4.93 mmol, 1.5 eq.) and 3-cyclopropyl-5-fluorophenol (500 mg, 3.29 mmol, 1 eq.) in MeCN (10 mL) was added K₂CO₃ (1.36 g, 9.86 mmol, 3 eq.) under N₂ atmosphere. The mixture was stirred at 80° C. for 12 h under N₂ atmosphere. Then the mixture was cooled, concentrated, and water (80 mL) was added. The mixture was extracted with EtOAc (30 mL×3). The combined organic layers were washed with brine (80 mL), dried over Na₂SO₄, filtered and concentrated. The residue was purified by flash silica gel chromatography (hexane to hexane/EtOAc=3/1, TLC: hexane/EtOAc=8/1, R_{f}=0.45) to give 2-bromo-4-(3-cyclopropyl-5-fluorophenoxy)pyridine (800 mg, 2.34 mmol, 71.11% yield) as yellow oil. LCMS [M+H]⁺=308.0; ¹H NMR (400 MHz, DMSO-d₆) δ=8.28 (d, J=5.6 Hz, 1H), 7.19 (d, J=2.0 Hz, 1H), 7.00 (dd, J=2.4, 5.6 Hz, 1H), 6.94 (td, J=2.4, 9.6 Hz, 1H), 6.89 (td, J=1.6, 10.0 Hz, 1H), 6.85 (s, 1H), 2.10-1.89 (m, 1H), 1.06-0.91 (m, 2H), 0.84-0.72 (m, 2H).

### Intermediate 54: Synthesis of 2-bromo-4-((2-chloropyridin-4-yl)oxy)pyridine

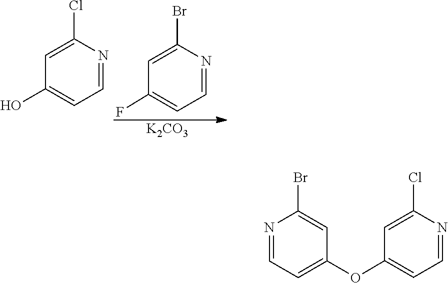

Step A: To a solution of 2-chloropyridin-4-ol (2 g, 15.44 mmol, 1 eq.) and 2-bromo-4-fluoropyridine (2.72 g, 15.44 mmol, 1 eq.) in DMF (5 mL) was added K₂CO₃ (2.13 g, 15.44 mmol, 1 eq.). The mixture was stirred at 80° C. for 16 h. The reaction mixture was quenched by addition H₂O (80 mL) and extracted with EtOAc (80 mL×3). The combined organic layers were washed with brine (80 mL), dried over Na₂SO₄, filtered and concentrated under reduced pressure to give a residue, which was purified by flash silica gel chromatography (hexanes to hexanes/EtOAc=70/30, TLC: hexanes/EtOAc=4/1, R_{f}=0.60) to give 2-bromo-4-((2-chloropyridin-4-yl)oxy)pyridine (3.6 g, 12.61 mmol, 81.67% yield) as a white solid. LCMS [M+H]⁺=287.0; ¹H NMR (400 MHz, DMSO-d₆) δ=8.44-8.40 (m, 2H), 7.55 (d, J=2.0 Hz, 1H), 7.42 (d, J=2.4 Hz, 1H), 7.29-7.24 (m, 2H).

### Intermediate 55: Synthesis of 5-((2-bromopyridin-4-yl)oxy)-1,3-dimethyl-1H-indazole

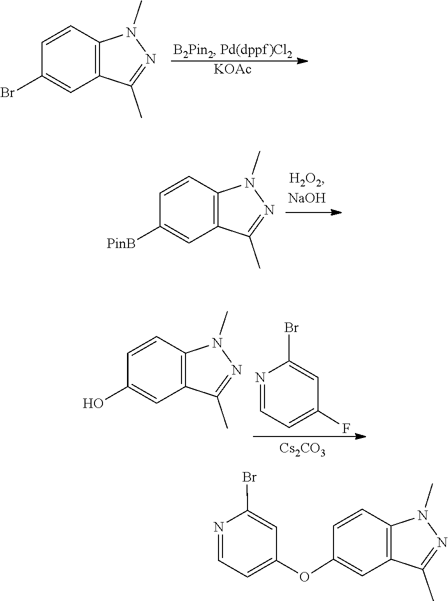

Step A: To a solution of 5-bromo-1,3-dimethyl-1H-indazole (0.80 g, 3.55 mmol, 1 eq.) in dioxane (16 mL) was added B₂Pin₂ (bis(pinacolato)diboron, 2.71 g, 10.66 mmol, 3 eq.), Pd(dppf)Cl₂ (0.10 g, 136.67 μmol, 0.04 eq.) and KOAc (1.05 g, 10.66 mmol, 3 eq.). The mixture was degassed and purged with N₂ three times, then the mixture was stirred at 100° C. for 1 h under N₂ atmosphere. Next, the reaction mixture was cooled and concentrated under reduced pressure to remove solvent to give a residue. The residue was purified by column chromatography (SiO₂, hexanes/EtOAc=100/1 to 3/1) to give 1,3-dimethyl-5-(4,4,5,5-tetramethyl-1,3,2-dioxaborolan-2-yl)-1H-indazole (0.90 g, 3.11 mmol, 87.46% yield) as a white solid. LCMS [M+H]⁺=273.1; ¹H NMR (400 MHz, DMSO-d₆) δ=8.05 (s, 1H), 7.62 (d, J=8.8 Hz, 1H), 7.51 (d, J=8.4 Hz, 1H), 3.94 (s, 3H), 2.45-2.64 (m, 3H), 1.30 (s, 12H).

Step B: To a solution of 1,3-dimethyl-5-(4,4,5,5-tetramethyl-1,3,2-dioxaborolan-2-yl)-1H-indazole (0.90 g, 3.11 mmol, 1 eq.) in THF (10 mL) and H₂O (2 mL) was added NaOH (0.60 g, 15.00 mmol, 4.83 eq.) and H₂O₂ (1.52 g, 13.41 mmol, 1.29 mL, 30% purity, 4.31 eq.) at 20° C., the mixture was stirred at 20° C. for 1 h. The reaction mixture was quenched by addition aq. Na₂SO₃ (100 mL) at 0° C., and then extracted with DCM (50 mL×3). The combined organic layers were dried over Na₂SO₄, filtered and concentrated under reduced pressure to give 1,3-dimethyl-1H-indazol-5-ol (0.72 g, crude) as colorless oil, which was used in the next step without further purification. LCMS [M+H]⁺=163.1; ¹H NMR (400 MHz, DMSO-d₆) δ=9.07 (s, 1H), 7.35 (d, J=8.8 Hz, 1H), 6.91 (d, J=8.8 Hz, 1H), 6.88 (d, J=8.0 Hz, 1H), 3.87 (s, 3H), 2.37 (s, 3H).

Step C: A mixture of 1,3-dimethyl-1H-indazol-5-ol (360 mg, 2.22 mmol, 1 eq.) and 2-bromo-4-fluoropyridine (468 mg, 2.66 mmol, 1.2 eq.) in DMF (4 mL) was added Cs₂CO₃ (2.17 g, 6.66 mmol, 3 eq.). The mixture was degassed and purged with N₂ three times, and then the mixture was stirred at 90° C. for 12 h under N₂ atmosphere. Then, the residue was cooled and diluted with EtOAc (100 mL) and extracted with water (100 mL). The combined organic layers were dried over sodium sulfate, filtered and concentrated under reduced pressure to give a residue. The residue was purified by column chromatography (SiO₂, hexanes/EtOAc=100/1 to 4/1) to give 5-((2-bromopyridin-4-yl)oxy)-1,3-dimethyl-1H-indazole (390 mg, 1.16 mmol, 52.46% yield) as red oil. LCMS [M+H]⁺=318.1; ¹H NMR (400 MHz, CDCl₃) δ=8.20 (d, J=6.0 Hz, 1H), 7.42-7.34 (m, 2H), 7.11 (dd, J=2.4, 4.8 Hz, 1H), 6.93 (d, J=2.0 Hz, 1H), 6.82 (dd, J=2.4, 6.0 Hz, 1H), 4.05 (s, 3H), 2.56 (s, 3H).

### Intermediate 56: Synthesis of tert-butyl 6-((2-bromopyridin-4-yl)oxy)-2,3-dihydro-4H-benzo[b][1,4]oxazine-4-carboxylate

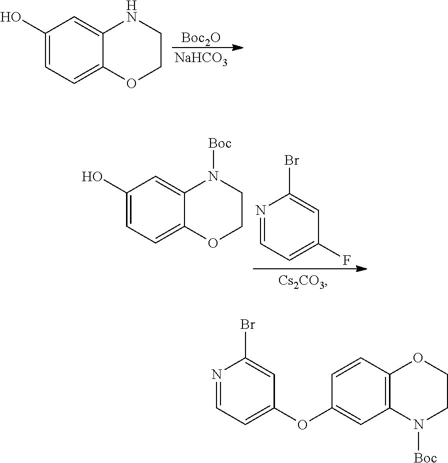

Step A: To a solution of 3,4-dihydro-2H-benzo[b][1,4]oxazin-6-ol (200 mg, 1.32 mmol, 1 eq.) in dioxane (4 mL) was added H₂O (4 mL), NaHCO₃ (333.44 mg, 3.97 mmol, 154.44 μL, 3 eq.) and Boc₂O (433.14 mg, 1.98 mmol, 455.93 u L, 1.5 eq.). The mixture was stirred at 20° C. for 16 h under N₂ atmosphere. Then, the reaction mixture was diluted with ethyl acetate and washed with water, filtered and concentrated under reduced pressure to give a residue. The residue was purified by flash silica gel chromatography (0~20% EtOAc/n-hexane gradient) to give tert-butyl 6-hydroxy-2,3-dihydro-4H-benzo[b][1,4]oxazine-4-carboxylate (112 mg, 445.72 μmol, 33.69% yield) as a white solid: ¹H NMR (400 MHz, CDCl₃) δ=8.88 (s, 1H), 7.33 (br s, 1H), 6.63 (d, J=8.8 Hz, 1H), 6.37 (dd, J=2.8, 8.8 Hz, 1H), 4.11-4.07 (m, 2H), 3.77-3.68 (m, 2H), 1.48 (s, 9H).

Step B: To a solution of tert-butyl 6-hydroxy-2,3-dihydro-4H-benzo[b][1,4]oxazine-4-carboxylate (105 mg, 417.86 μmol, 1 eq.) in DMF (5 mL) was added Cs₂CO₃ (408.44 mg, 1.25 mmol, 3 eq.) and 2-bromo-4-fluoropyridine (88.25 mg, 501.44 μmol, 1.2 eq.). The mixture was stirred at 90° C. for 5 h under N₂ atmosphere. The mixture was filtered and concentrated under reduced pressure to give a residue. The residue was purified by flash silica gel chromatography (0~12% ethyl acetate/n-hexane gradient) to give tert-butyl 6-((2-bromopyridin-4-yl)oxy)-2,3-dihydro-4H-benzo[b][1,4]oxazine-4-carboxylate (106 mg, 234.25 μmol, 56.06% yield) as colorless oil. LCMS [M+H]⁺=409.0; ¹H NMR (400 MHz, DMSO-d₆) δ=8.25 (d, J=5.6 Hz, 1H), 7.69-7.59 (m, 1H), 7.06 (d, J=2.4 Hz, 1H), 7.00-6.94 (m, 2H), 6.86 (dd, J=2.8, 8.8 Hz, 1H), 4.29-4.15 (m, 2H), 3.84-3.79 (m, 2H), 1.43 (s, 9H).

### Intermediate 57: Synthesis of 6-((2-bromopyridin-4-yl)oxy)-4-fluoro-3-methyl-1-(tetrahydro-2H-pyran-2-yl)-1H-indazole

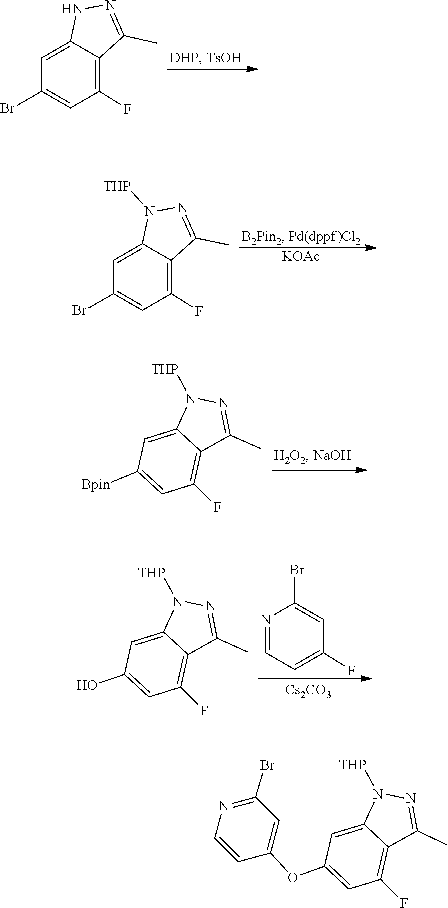

Step A: To a solution of 6-bromo-4-fluoro-3-methyl-1H-indazole (620 mg, 2.71 mmol, 1 eq.) and DHP (dihydropyran, 683.07 mg, 8.12 mmol, 742.47 u L, 3 eq.) in toluene (10 mL) was added TsOH (46.61 mg, 270.68 u mol, 0.1 eq.). The mixture was stirred at 100° C. for 6 h. The reaction mixture was diluted with DCM (100 mL). The organic layer was dried over anhydrous sodium sulfate, filtered and concentrated under vacuum to obtain the crude product. The residue was purified by flash silica gel chromatography (0-12% ethyl acetate/Petroleum ether gradient) to give 6-bromo-4-fluoro-3-methyl-1-(tetrahydro-2/I-pyran-2-yl)-1H-indazole (620 mg, 1.94 mmol, 71.68% yield) as a white solid: ¹H NMR (400 MHz, CDCl₃) δ=7.48 (d, J=0.8 Hz, 1H), 6.92 (dd, J=1.2, 9.6 Hz, 1H), 5.54 (dd, J=2.4, 9.6 Hz, 1H), 4.13-4.01 (m, 1H), 3.82-3.66 (m, 1H), 2.64 (s, 3H), 2.58-2.40 (m, 1H), 2.21-1.97 (m, 2H), 1.81-1.62 (m, 3H).

Step B: To a solution of 6-bromo-4-fluoro-3-methyl-1-(tetrahydro-2H-pyran-2-yl)-1H-indazole (620 mg, 1.98 mmol, 1 eq.) and B₂Pin₂ (754.11 mg, 2.97 mmol, 1.5 eq.) in dioxane (10 mL) was added KOAc (388.59 mg, 3.96 mmol, 2 eq.) and Pd(dppf) C12 (72.43 mg, 98.99 u mol, 0.05 eq.). The mixture was stirred at 100° C. for 16 h. Then, the mixture was filtered and concentrated under reduced pressure to give a residue. The residue was purified by flash silica gel chromatography (0~10% Ethyl acetate/n-Hexane gradient) to give 4-fluoro-3-methyl-1-(tetrahydro-2H-pyran-2-yl)-6-(4,4,5,5-tetramethyl-1,3,2-dioxaborolan-2-yl)-1H-indazole (750 mg, 1.67 mmol, 84.13% yield) as colorless oil. LCMS [M+H]⁺=361.1; ¹H NMR (400 MHz, CDCl₃) δ=7.70 (s, 1H), 7.53 (s, 1H), 7.14 (d, J=10.4 Hz, 1H), 5.67 (dd, J=2.8, 10.4 Hz, 1H), 4.10 (br d, J=10.8 Hz, 1H), 3.85-3.69 (m, 1H), 2.68 (s, 3H), 2.61-2.54 (m, 1H), 2.23-2.06 (m, 1H), 2.03-1.92 (m, 1H), 1.82-1.68 (m, 2H), 1.38 (s, 12H).

Step C: To a solution of 4-fluoro-3-methyl-1-(tetrahydro-2H-pyran-2-yl)-6-(4,4,5,5-tetramethyl-1,3,2-dioxaborolan-2-yl)-1H-indazole (700 mg, 1.94 mmol, 1 eq.) in THF (15 mL) was added H₂O₂ (3.32 g, 30.22 mmol, 2.81 mL, 31% purity, 15.55 eq.) and aq. NaOH (1 M, 1.94 mL, 1 eq.) at 0° C. The mixture was stirred at 0° C. for 1 h. Then, the reaction mixture was quenched by addition of aq. Na₂SO₃ (150 mL) at 0° C., and extracted with DCM (100 mL×3). The combined organic layers were dried over Na₂SO₄, filtered and concentrated under reduced pressure to give a residue. The residue was purified by flash silica gel chromatography (0-25% ethyl acetate/petroleum ether gradient) to give 4-fluoro-3-methyl-1-(tetrahydro-2H-pyran-2-yl)-1H-indazol-6-ol (490 mg, 1.57 mmol, 80.60% yield) as a white solid: ¹H NMR (400 MHz, DMSO-d₆) δ=10.02 (s, 1H), 6.69 (d, J=1.6 Hz, 1H), 6.40 (dd, J=1.6, 12.0 Hz, 1H), 5.55 (dd, J=2.4, 9.6 Hz, 1H), 3.86 (br d, J=10.8 Hz, 1H), 3.71-3.61 (m, 1H), 2.46 (s, 3H), 2.37-2.26 (m, 1H), 2.03-1.95 (m, 1H), 1.93-1.85 (m, 1H), 1.78-1.64 (m, 1H), 1.59-1.48 (m, 2H).

Step D: To a solution of 4-fluoro-3-methyl-1-(tetrahydro-2H-pyran-2-yl)-1H-indazol-6-ol (600 mg, 2.40 mmol, 1 eq.) and 2-bromo-4-fluoropyridine (506.30 mg, 2.88 mmol, 1.2 eq.) in DMF (8 mL) was added Cs₂CO₃ (2.34 g, 7.19 mmol, 3 eq.). The mixture was stirred at 90° C. for 16 h. The reaction mixture was concentrated under vacuum. Then the residue was diluted with EtOAc (15 mL×3), washed with H₂O (5 mL) and brine (5 mL). The organic layer was dried over Na₂SO₄, filtered and concentrated under vacuum to dryness to give a crude product. The residue was purified by flash silica gel chromatography (0-25% ethyl acetate/petroleum ether gradient) to give 6-((2-bromopyridin-4-yl)oxy)-4-fluoro-3-methyl-1-(tetrahydro-2H-pyran-2-yl)-1H-indazole (800 mg, 1.58 mmol, 65.71% yield) as a white solid. LCMS [M+H]⁺=407.7; ¹H NMR (400 MHz, CDCl₃) δ=8.25 (d, J=6.0 Hz, 1H), 7.04 (dd, J=2.0, 3.2 Hz, 2H), 6.88 (dd, J=2.0, 5.6 Hz, 1H), 6.59-6.49 (m, 1H), 5.60-5.48 (m, 1H), 4.10-4.01 (m, 1H), 3.71 (dt, J=2.8, 11.2 Hz, 1H), 2.67 (s, 3H), 2.57-2.45 (m, 1H), 2.17-2.09 (m, 1H), 2.07-2.02 (m, 1H), 1.80-1.71 (m, 2H), 1.67-1.62 (m, 1H); ¹⁹F NMR (376 MHz, CDCl₃) δ=−116.033.

Intermediate 58: Synthesis of 1,3-dimethyl-1,5,6,7-tetrahydro-4H-pyrazolo[4,3-c]pyridin-4-one

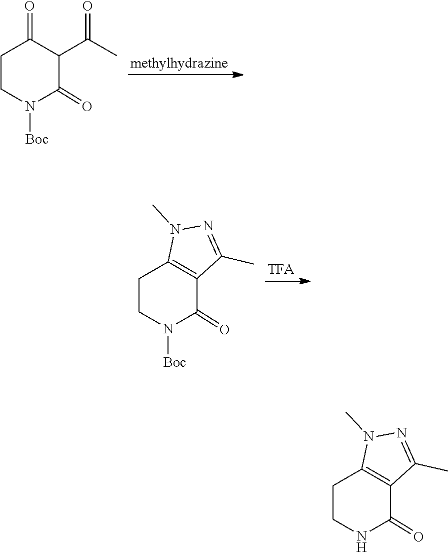 [SMILES: CC(=O)C1C(=O)CCN(C(=O)OC(C)(C)C)C1=O ; Cc1nn(C)c2c1C(=O)N(C(=O)OC(C)(C)C)CC2 ; Cc1nn(C)c2c1C(=O)NCC2]

Step A: A mixture of tert-butyl 3-acetyl-2,4-dioxopiperidine-1-carboxylate (500 mg, 1.96 mmol, 1 eq.) in EtOH (10 mL) was added methylhydrazine (676.81 mg, 5.88 mmol, 3 eq.). The mixture was degassed and purged with N₂ three times, then the mixture was stirred at 20° C. for 3 h under N₂ atmosphere. The reaction mixture was quenched by sat. brine (5 mL) at 20° C. and extracted with DCM (50 mL×3). The combined organic layers were dried over Na₂SO₄, filtered, and concentrated under reduced pressure to give a residue.

The residue was purified by column chromatography (SiO₂, hexanes/EtOAc/TEA=100/1/0.01 to 4/1/0.01) to give tert-butyl 1,3-dimethyl-4-oxo-1,4,6,7-tetrahydro-5H-pyrazolo[4,3-c]pyridine-5-carboxylate (90 mg, 332.44 μmol, 16.97% yield) as colorless oil. LCMS [M+H]⁺=210.1; ¹H NMR (400 MHz, CDCl₃) δ=4.09 (t, J=6.4 Hz, 2H), 3.73 (s, 3H), 2.85 (t, J=6.4 Hz, 2H), 2.47 (s, 3H), 1.57 (s, 9H).

Step B: To a solution of tert-butyl 1,3-dimethyl-4-oxo-1,4,6,7-tetrahydro-5H-pyrazolo[4,3-c]pyridine-5-carboxylate (65 mg, 245.00 μmol, 1 eq.) in DCM (4 mL) was added TFA (83.81 mg, 735.00 μmol, 3 eq.) at 20° C., the mixture was stirred at 20° C. for 2 h. Then, the reaction mixture was concentrated under reduced pressure to give 1,3-dimethyl-1,5,6,7-tetrahydro-4H-pyrazolo[4,3-c]pyridin-4-one (40 mg, crude) as colorless oil, which was used in the next step without further purification. LCMS [M+H]⁺=166.1.

Intermediate 59: Synthesis of 6-((2-bromopyridin-4-yl)methyl)-3-(trifluoromethyl)pyrazolo[1,5-a]pyridine

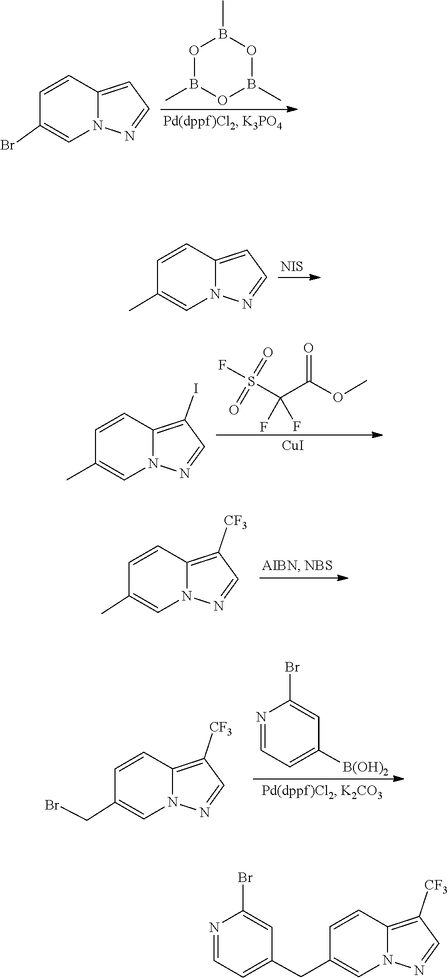

Step A: To a solution of 6-bromopyrazolo[1,5-a]pyridine (5 g, 25.38 mmol, 1 eq.) and trimethylboroxine (3 M, 42.29 mL, 5 eq.) in dioxane (120 mL) and H₂O (40 mL) was added Pd(dppf)Cl₂ (928.40 mg, 1.27 mmol, 0.05 eq.) and K₃PO₄ (10.77 g, 50.75 mmol, 2 eq.) under N₂ atmosphere. The mixture was stirred at 80° C. for 16 h under N₂ atmosphere. Then, the reaction mixture was cooled and concentrated under vacuum. The residue was diluted with EtOAc (100 mL×3), washed with H₂O (50 mL) and brine (50 mL). The organic layer was dried over Na₂SO₄, filtered and concentrated under vacuum to dryness to give a crude product. The residue was purified by flash silica gel chromatography (hexanes to EtOAc/hexanes =10/90) to yield 6-methylpyrazolo[1,5-a]pyridine (1.26 g, 8.10 mmol, 31.93% yield) as yellow oil: ¹H NMR (400 MHz, CDCl₃) δ=8.28 (s, 1H), 7.88 (d, J=2.0 Hz, 1H), 7.45 (d, J=9.2 Hz, 1H), 6.96 (d, J=9.2 Hz, 1H), 6.46 (d, J=2.0 Hz, 1H), 2.34 (s, 3H).

Step B: To a solution of 6-methylpyrazolo[1,5-a]pyridine (1.26 g, 9.53 mmol, 1 eq.) in MeCN (20 mL) was added NIS (N-iodosuccinimide, 3.22 g, 14.30 mmol, 1.5 eq.). The mixture was stirred at 20° C. for 1 h. The reaction mixture was concentrated under vacuum and the residue was diluted with EtOAc (80 mL×3), washed with H₂O (30 mL) and brine (30 mL). The organic layer was dried over Na₂SO₄, filtered and concentrated under vacuum to dryness to give a crude product. The residue was purified by flash silica gel chromatography (hexanes to EtOAc/hexanes=10/90) to yield 3-iodo-6-methylpyrazolo[1,5-a]pyridine (1.53 g, 4.74 mmol, 49.75% yield) as a yellow solid: ¹H NMR (400 MHz, CDCl₃) δ=8.26 (s, 1H), 7.89 (s, 1H), 7.38 (d, J=8.8 Hz, 1H), 7.07 (d, J=9.2 Hz, 1H), 2.37 (s, 3H).

Step C: To a solution of 3-iodo-6-methylpyrazolo[1,5-a]pyridine (300 mg, 1.16 mmol, 1 eq.) in NMP (5 mL) was added CuI (265.68 mg, 1.40 mmol, 1.2 eq.) and methyl 2,2-difluoro-2-(fluorosulfonyl)acetate (670.01 mg, 3.49 mmol, 443.72 μL, 3 eq.) under N₂ atmosphere. The mixture was stirred at 110° C. for 3 h under microwave irradiation. The reaction mixture was concentrated under vacuum, then residue was diluted with EtOAc (50 mL×3), washed with H₂O (30 mL) and brine (30 mL). The organic layer was dried over Na₂SO₄, filtered and concentrated under vacuum to dryness to give a crude product. The residue was purified by flash silica gel chromatography (hexanes to EtOAc/hexanes=10/90) to yield 6-methyl-3-(trifluoromethyl) pyrazolo[1,5-a]pyridine (82 mg, 0.36 mmol, 31.59% yield) as a yellow solid. LCMS [M+H]⁺=200.9; ¹H NMR (400 MHz, CDCl₃) δ=8.34 (s, 1H), 8.10 (s, 1H), 7.63 (d, J=9.2 Hz, 1H), 7.22 (d, J=9.2 Hz, 1H), 2.41 (s, 3H); ¹⁹F NMR (376 MHz, CDCl₃) δ=−55.451.

Step D: To a solution of 6-methyl-3-(trifluoromethyl) pyrazolo[1,5-a]pyridine (350 mg, 1.75 mmol, 1 eq.) in CCl₄ (5 mL) was added NBS (342.34 mg, 1.92 mmol, 1.1 eq.) and AIBN (28.71 mg, 174.86 μmol, 0.1 eq.). The mixture was stirred at 80° C. for 4 h. Then reaction mixture was cooled and diluted with EtOAc (10 mL×5), washed with aq. Na₂CO₃ (4 mL) and brine (4 mL). The organic layer was dried over Na₂SO₄, filtered and concentrated under vacuum to dryness to give a crude product. The residue was purified by flash silica gel chromatography (hexanes to EtOAc/hexanes=5/95) to yield 6-(bromomethyl)-3-(trifluoromethyl) pyrazolo[1,5-a]pyridine (210 mg, 526.78 μmol, 30.13% yield) as a yellow solid. LCMS [M+H]⁺=278.9; ¹H NMR (400 MHz, CDCl₃) δ=8.55 (s, 1H), 8.17 (s, 1H), 7.72 (d, J=9.6 Hz, 1H), 7.44-7.37 (m, 1H), 4.53 (s, 2H).

Step E: To a solution of 6-(bromomethyl)-3-(trifluoromethyl) pyrazolo[1,5-a]pyridine (210 mg, 752.54 μmol, 1 eq.) and (2-bromopyridin-4-yl) boronic acid (182.25 mg, 903.04 μmol, 1.2 eq.) in dioxane (2 mL) and H₂O (0.5 mL) was added Pd(dppf) C12 (55.06 mg, 75.25 μmol, 0.1 eq.) and K₂CO₃ (208.01 mg, 1.51 mmol, 2 eq.). The mixture was stirred at 65° C. for 1 h. Then, the reaction mixture was cooled and filtered and the filtrate was concentrated under vacuum to dryness to give a crude product. The residue was purified by flash silica gel chromatography (hexanes to EtOAc/hexanes=20/80) to yield 6-((2-bromopyridin-4-yl)methyl)-3-(trifluoromethyl) pyrazolo[1,5-a]pyridine (130 mg, 328.52 μmol, 43.66% yield) as a white solid. LCMS [M+H]⁺=357.8; ¹H NMR (400 MHz, CDCl₃) δ=8.38 (s, 1H), 8.33 (d, J=5.2 Hz, 1H), 8.16 (s, 1H), 7.69 (d, J=9.2 Hz, 1H), 7.36 (s, 1H), 7.17-7.10 (m, 2H), 3.99 (s, 2H); ¹⁹F NMR (376 MHz, CDCl₃) δ=−55.535.

### Intermediate 60: Synthesis of 3-((benzyloxy)methyl)-1-methyl-1,5,6,7-tetrahydro-4H-pyrazolo[4,3-c]pyridin-4-one

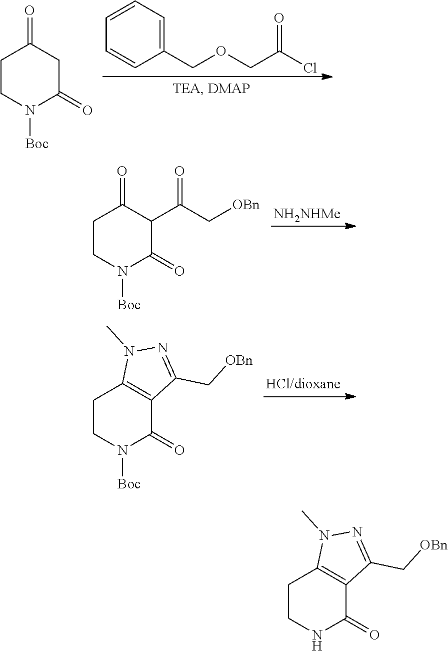

Step A: To a solution of tert-butyl 2,4-dioxopiperidine-1-carboxylate (1 g, 4.69 mmol, 1 eq.), TEA (711.84 mg, 7.03 mmol, 979.14 μL, 1.5 eq.) and DMAP (171.88 mg, 1.41 mmol, 0.3 eq.) in DCM (30 mL) was added benzyl chloroformate (1.04 g, 5.63 mmol, 873.83 μL, 1.2 eq.) at 0° C. under N₂. The mixture was stirred at 20° C. for 16 h. Then, the reaction mixture was quenched by addition H₂O (20 mL) and extracted with EtOAc (30 mL×3). The combined organic layers were washed with brine (30 mL), dried over Na₂SO₄, filtered and concentrated under reduced pressure to give a residue, which was purified by flash silica gel chromatography (hexanes to hexanes/EtOAc=60/40, TLC: hexane/EtOAc=0/1, R_{f}=0.30) to yield tert-butyl 3-(2-(benzyloxy) acetyl)-2,4-dioxopiperidine-1-carboxylate (770 mg, 2.02 mmol, 43.16% yield) as a white solid: ¹H NMR (400 MHz, DMSO-d₆) δ=7.40-7.25 (m, 5H), 4.75 (s, 2H), 4.57 (s, 2H), 3.76 (t, J=6.0 Hz, 2H), 3.44-3.34 (m, 1H), 2.80 (s, 2H), 1.46 (s, 9H).

Step B: To a solution of tert-butyl 3-(2-(benzyloxy) acetyl)-2,4-dioxopiperidine-1-carboxylate (670 mg, 1.76 mmol, 1 eq.) in EtOH (30 mL) was added methylhydrazine (1.54 g, 13.37 mmol, 1.76 mL, 7.59 eq.). The mixture was stirred at 20° C. for 5 h. The mixture was diluted with HCl (1 N, 5 mL), the resulting mixture was extracted by EtOAc (30 mL×2). The combined organic layer was extracted with sat. NaCl (50 mL×2). The combined organic layer was dried over anhydrous sodium sulfate and concentrated under reduced pressure to afford the crude product, which was purified by flash silica gel chromatography (hexane to hexane/EtOAc=60/40, TLC: hexane/EtOAc=50/50, R_{f}=0.4) to yield tert-butyl 3-((benzyloxy)methyl)-1-methyl-4-oxo-1,4,6,7-tetrahydro-5H-pyrazolo[4,3-c]pyridine-5-carboxylate (500 mg, 1.28 mmol, 72.61% yield) as a white solid: ¹H NMR (400 MHz, DMSO-d₆) δ=7.37-7.24 (m, 5H), 4.63 (s, 2H), 4.54 (s, 2H), 3.95 (t, J=6.4 Hz, 2H), 3.75 (s, 3H), 2.96 (t, J=6.0 Hz, 2H), 1.47 (s, 9H).

Step C: To a solution of tert-butyl 3-((benzyloxy)methyl)-1-methyl-4-oxo-1,4,6,7-tetrahydro-5H-pyrazolo[4,3-c]pyridine-5-carboxylate (200 mg, 511.54 μmol, 1 eq.) in DCM (10 mL) was added HCl/dioxane (2 M, 10 mL, 39.10 eq.). The mixture was stirred at 20° C. for 2 h. Then, the reaction mixture was concentrated under reduced pressure to give 3-((benzyloxy)methyl)-1-methyl-1,5,6,7-tetrahydro-4H-pyrazolo[4,3-c]pyridin-4-one (160 mg, crude) as a white HCl salt, which was used directly in the next step without further purification: ¹H NMR (400 MHz, DMSO-d₆) δ=7.37-7.30 (m, 4H), 7.30-7.25 (m, 1H), 7.21 (s, 1H), 4.62 (s, 2H), 4.53 (s, 2H), 3.74 (s, 3H), 3.39 (dt, J=2.8, 6.8 Hz, 2H), 2.86 (t, J=6.8 Hz, 2H).

Intermediate 61: Synthesis of 6-((2-bromopyridin-4-yl)methyl)-3-(trifluoromethyl)benzo[d]isoxazole

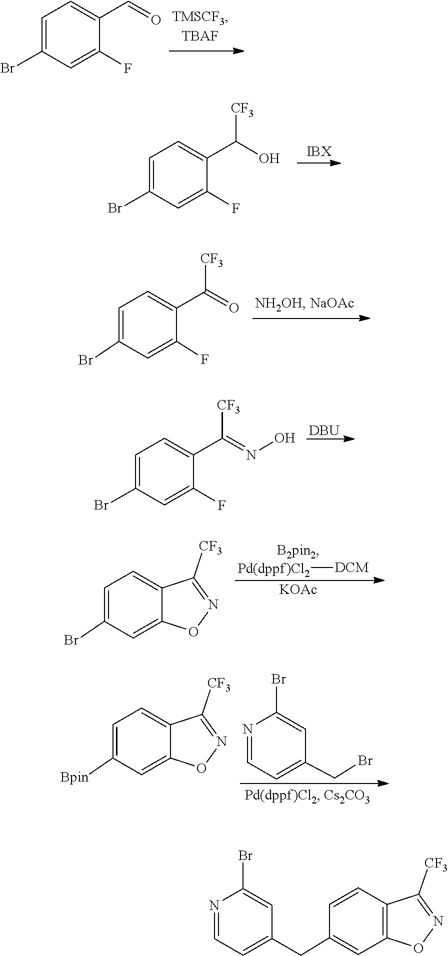

Step A: To a solution of 4-bromo-2-fluorobenzaldehyde (3 g, 14.78 mmol, 1 eq.) in THF (15 mL) was added TMSCF₃ (2.52 g, 17.73 mmol, 1.2 eq.) and TBAF (1 M, 2.96 mL, 0.2 eq.) at 0° C. The mixture was stirred at 25° C. for 3 h. The reaction mixture was quenched by HCl (1 M, 50 mL), then diluted with H₂O (100 mL), and extracted with EtOAc (200 mL×3). The combined organic layers were washed with brine (200 mL×3), dried over Na₂SO₄, filtered and concentrated under reduced pressure to yield 1-(4-bromo-2-fluorophenyl)-2,2,2-trifluoroethan-1-ol (4 g, crude) as yellow oil, which was used into the next step without further purification: ¹H NMR (400 MHz, DMSO-d₆) δ=7.66-7.60 (m, 1H), 7.59-7.51 (m, 2H), 7.14 (d, J=6.0 Hz, 1H), 5.4-5.30 (m, 1H); ¹⁹F NMR (376 MHz, DMSO-d₆) δ=−77.429, −114.732.

Step B: To a solution of 1-(4-bromo-2-fluorophenyl)-2,2,2-trifluoroethan-1-ol (4 g, 14.65 mmol, 1 eq.) in EtOAc (120 mL) was added IBX (8.20 g, 29.30 mmol, 2 eq.). The mixture was stirred at 80° C. for 12 h. The reaction mixture was cooled and filtered, then concentrated under reduced pressure to yield 1-(4-bromo-2-fluorophenyl)-2,2,2-trifluoroethan-1-one (3.95 g, crude) as yellow oil, which was used in the next step without further purification: ¹H NMR (400 MHz, CDCl₃) δ=7.83-7.74 (m, 1H), 7.53-7.43 (m, 2H); ¹⁹F NMR (376 MHz, CDCl₃) δ=−74.884, −105.024.

Step C: To a solution of 1-(4-bromo-2-fluorophenyl)-2,2,2-trifluoroethan-1-one (3.9 g, 14.39 mmol, 1 eq.) in MeOH (40 mL) was added NH₂OH·HCl (8.00 g, 115.13 mmol, 8 eq.) and NaOAc (11.80 g, 143.91 mmol, 10 eq.). The mixture was stirred at 70° C. for 12 h. Then, the reaction mixture was cooled, filtered and concentrated under reduced pressure to give a residue. Then the residue was diluted with H₂O (100 mL) and extracted with EtOAc (450 mL×3). The combined organic layers were dried over Na₂SO₄, filtered and concentrated under reduced pressure to yield 1-(4-bromo-2-fluorophenyl)-2,2,2-trifluoroethan-1-one oxime (4.6 g, crude) as yellow oil, which was used in the next step without further purification: ¹H NMR (400 MHz, CDCl₃) δ=8.93 (br s, 1H), 7.46-7.39 (m, 2H), 7.23-7.17 (m, 1H).

Step D: 1-(4-bromo-2-fluorophenyl)-2,2,2-trifluoroethan-1-one oxime (2 g, 6.99 mmol, 1 eq.) and DBU (745.16 mg, 4.89 mmol, 737.78 μL, 0.7 eq.) were dissolved in THF (20 mL) in a microwave tube. The sealed tube was heated at 110° C. for 30 min under microwave. Then reaction mixture was filtered and concentrated under reduced pressure to give a residue. The residue was purified by flash silica gel chromatography (hexanes to hexanes/EtOAc=10/1, TLC: hexanes/EtOAc=5/1, R_{f}=0.6) to yield 6-bromo-3-(trifluoromethyl)benzo[d]isoxazole (400 mg, 1.27 mmol, 90.98% yield) as yellow oil: ¹H NMR (400 MHz, CDCl₃) δ=7.92 (d, J=2.8 Hz, 1H), 7.77-7.66 (m, 1H), 7.65-7.53 (m, 1H); ¹⁹F NMR (376 MHz, CDCl₃) δ=−62.419.

Step E: A mixture of 6-bromo-3-(trifluoromethyl)benzo[d] (1.1 g, 4.14 mmol, 1 eq.), 4,4,5,5-tetramethyl-2-(4,4,5,5-tetramethyl-1,3,2-dioxaborolan-2-yl)-1,3,2-dioxaborolane (1.58 g, 6.20 mmol, 1.5 eq.), KOAc (811.66 mg, 8.27 mmol, 2 eq.), Pd(dppf)Cl₂·CH₂Cl₂ (675.38 mg, 827.02 μmol, 0.2 eq.) in dioxane (10 mL) was degassed and purged with N₂ three times, and then the mixture was stirred at 80° C. for 12 h under N₂ atmosphere. The reaction mixture was cooled and filtered, then concentrated under reduced pressure to give a residue. The residue was purified by flash silica gel chromatography (hexanes to hexanes/EtOAc=25/1, TLC: hexanes/EtOAc=10/1, R_{f}=0.5) to yield 6-(4,4,5,5-tetramethyl-1,3,2-dioxaborolan-2-yl)-3-(trifluoromethyl)benzo[d]isoxazole (950 mg, 3.03 mmol, 73.38% yield) as yellow oil: ¹H NMR (400 MHz, CDCl₃) δ=8.15 (s, 1H), 7.89-7.84 (m, 1H), 7.84-7.79 (m, 1H), 1.39 (s, 12H).

Step F: A mixture of 6-(4,4,5,5-tetramethyl-1,3,2-dioxaborolan-2-yl)-3-(trifluoromethyl)benzo[d]isoxazole (300 mg, 958.22 μmol, 1 eq.), 2-bromo-4-(bromomethyl)pyridine (312.57 mg, 1.25 mmol, 1.3 eq.), Cs₂CO₃ (624.41 mg, 1.92 mmol, 2 eq.), Pd(dppf)Cl₂ (70.11 mg, 95.82 μmol, 0.1 eq.) and H₂O (0.5 mL) in dioxane (5 mL) was degassed and purged with N₂ three times, then the mixture was stirred at 50° C. for 1 h under N₂ atmosphere. Next, reaction mixture was cooled and diluted with H₂O (10 mL) and extracted with EtOAc (15 mL×3). The combined organic layers were washed with brine (15 mL×2), dried over Na₂SO₄, filtered and concentrated under reduced pressure to give a residue. The residue was purified by flash silica gel chromatography (hexanes to hexanes/EtOAc=10/1, TLC: hexanes/EtOAc=5/1, R_{f}=0.5) to yield 6-((2-bromopyridin-4-yl)methyl)-3-(trifluoromethyl)benzo[d]isoxazole (160 mg, 448.02 μmol, 46.76% yield) as yellow oil: ¹H NMR (400 MHz, CDCl₃) δ=8.31 (d, J=5.2 Hz, 1H), 7.79 (d, J=8.0 Hz, 1H), 7.53 (s, 1H), 7.33 (s, 1H), 7.29 (d, J=8.4 Hz, 1H), 7.11-7.07 (m, 1H), 4.15 (s, 2H); ¹⁹F NMR (376 MHz, CDCl₃) δ=−62.450.

Intermediate 62: Synthesis of 3-ethyl-1-methyl-1,5,6,7-tetrahydro-4H-pyrazolo[4,3-c]pyridin-4-one

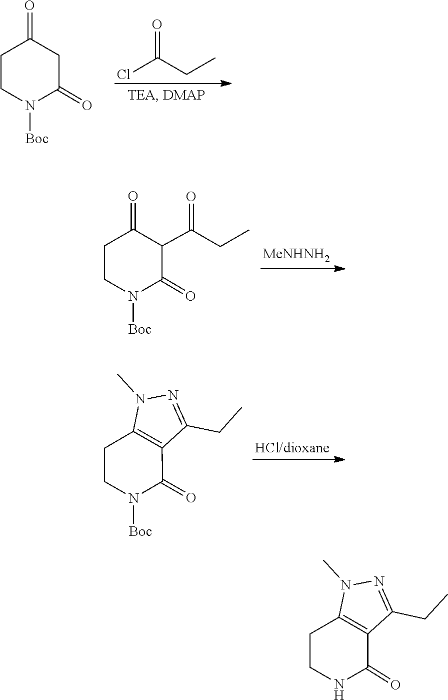

Step A: To a solution of tert-butyl 2,4-dioxopiperidine-1-carboxylate (2 g, 9.38 mmol, 1 eq.), TEA (1.42 g, 14.07 mmol, 1.96 mL, 1.5 eq.) and DMAP (343.77 mg, 2.81 mmol, 0.3 eq.) in DCM (30 mL) was added propionyl chloride (1.04 g, 11.26 mmol, 1.04 mL, 1.2 eq.) at 0° C. under N₂. The mixture was stirred at 20° C. for 16 h. The reaction mixture was quenched by addition H₂O (80 mL) and extracted with EtOAc (80 mL×3). The combined organic layers were washed with brine (80 mL), dried over Na₂SO₄, filtered, and concentrated under reduced pressure to give a residue, which was purified by flash silica gel chromatography (hexane to hexane/EtOAc=80/20, TLC: hexane/EtOAc=1/1, R_{f}=0.70) to yield tert-butyl 2,4-dioxo-3-propionylpiperidine-1-carboxylate (1.5 g, 5.29 mmol, 56.42% yield) as a white solid: ¹H NMR (400 MHz, CDCl₃) δ=3.89-3.83 (m, 2H), 3.14-3.06 (m, 2H), 2.70 (t, J=6.4 Hz, 2H), 1.57-1.55 (m, 9H), 1.20-1.14 (m, 3H).

Step B: To a solution of tert-butyl 2,4-dioxo-3-propionylpiperidine-1-carboxylate (500 mg, 1.76 mmol, 1 eq.) in EtOH (20 mL) was added methylhydrazine (2.12 g, 18.41 mmol, 2.42 mL, 40% purity, 10.44 eq.). The mixture was stirred at 35° C. for 2 h. Then, the mixture was cooled and diluted with HCl (1 N, 5 mL), the resulting mixture was extracted by EtOAc (30 mL×2). The combined organic layer was washed with sat. NaCl (50 mL×2). The combined organic layer was dried over anhydrous sodium sulfate, filtered, and concentrated under reduced pressure to afford the crude product, which was purified by flash silica gel chromatography (hexane to hexane/EtOAc=60/40, TLC: hexane/EtOAc=1/1, R_{f}=0.4) to yield tert-butyl 3-ethyl-1-methyl-4-oxo-1,4,6,7-tetrahydro-5H-pyrazolo[4,3-c]pyridine-5-carboxylate (460 mg, 1.48 mmol, 84.02% yield) as a white solid: LCMS [M+H]⁺=280.2; ¹H NMR (400 MHz, CDCl₃) δ=4.11-4.04 (m, 2H), 3.80-3.73 (m, 3H), 2.91-2.84 (m, 4H), 1.56 (s, 9H), 1.28-1.24 (m, 3H).

Step C: To a solution of tert-butyl 3-ethyl-1-methyl-4-oxo-1,4,6,7-tetrahydro-5H-pyrazolo[4,3-c]pyridine-5-carboxylate (160 mg, 544.15 μmol, 1 eq.) in DCM (2 mL) was added HCl/dioxane (2 M, 20.00 mL, 73.51 eq.). The mixture was stirred at 20° C. for 0.5 h.

The reaction mixture was concentrated under reduced pressure to give 3-ethyl-1-methyl-1,5,6,7-tetrahydro-4H-pyrazolo[4,3-c]pyridin-4-one (120 mg, crude) as a white solid, which was used directly in the next step without further purification.

### Intermediate 63: Synthesis of 3-(hydroxymethyl)-1-methyl-1,4,5,6-tetrahydro-7H-pyrazolo[3,4-c]pyridin-7-one

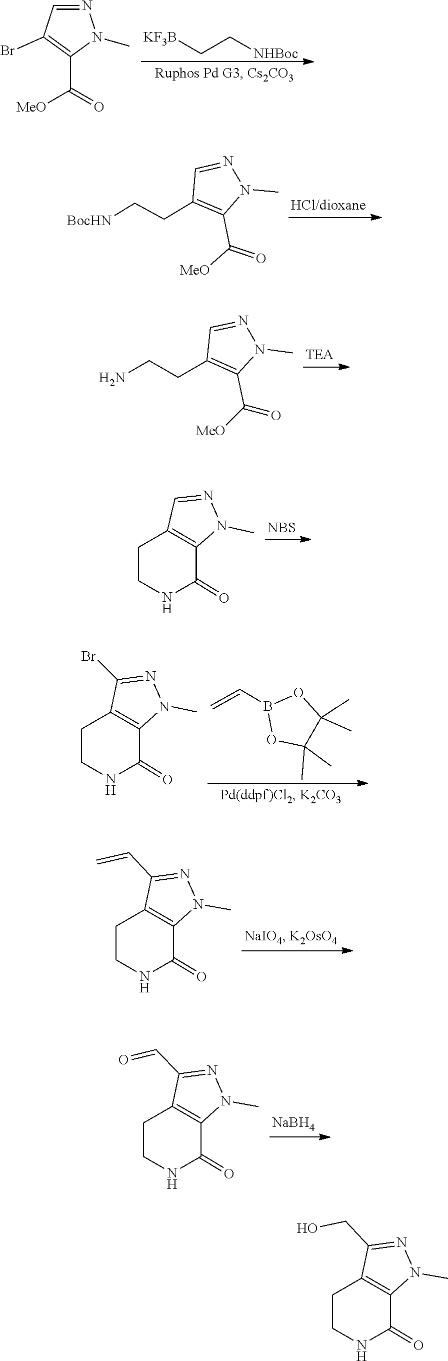

Step A: A mixture of methyl 4-bromo-1-methyl-1H-pyrazole-5-carboxylate (2 g, 9.13 mmol, 1 eq.), Potassium tert-butyl N-(2-(trifluoroboranuidyl)ethylcarbamate (3.44 g, 13.70 mmol, 1.5 eq.), Cs₂CO₃ (8.93 g, 27.39 mmol, 3 eq.) and RuPhos Pd G3 ((2-dicyclohexylphosphino-2′,6′-diisopropoxy-1,1′-biphenyl) [2-(2′-amino-1,1′-biphenyl)]palladium (II) methanesulfonate, CAS #1445085-77-7, 763.68 mg, 913.09 μmol, 0.1 eq.) in toluene (20 mL) and H₂O (5 mL) was degassed and purged with N₂ three times, and the mixture was stirred at 80° C. for 16 h under N₂ atmosphere. The reaction mixture was cooled and concentrated under vacuum, then residue was diluted with EtOAc (150 mL×3), washed with H₂O (200 mL) and brine (180 mL). The organic layer was dried over Na₂SO₄, filtered and concentrated under vacuum to dryness to give a crude product. The residue was purified by flash silica gel chromatography (hexanes to hexanes/EtOAc=30/70) to yield methyl 4-(2-((tert-butoxycarbonyl)amino)ethyl)-1-methyl-1H-pyrazole-5-carboxylate (2 g, 6.64 mmol, 72.67% yield) as a yellow solid. LCMS [M+H]⁺=284.0; ¹H NMR (400 MHz, CDCl₃) δ=7.35 (s, 1H), 4.62 (br s, 1H), 4.14 (s, 3H), 3.92 (s, 3H), 3.38-3.27 (m, 2H), 2.88 (t, J=6.8 Hz, 2H), 1.43 (s, 9H).

Step B: A mixture of methyl 4-(2-((tert-butoxycarbonyl)amino)ethyl)-1-methyl-1H-pyrazole-5-carboxylate (1.8 g, 6.35 mmol, 1 eq.) in HCl/dioxane (20 mL) was stirred at 25° C. for 2 h. The reaction mixture was concentrated under vacuum and the residue was diluted in DCM (5 mL), Na₂CO₃ (200 mg) was added into the mixture, the mixture was stirred for 30 min. Then, the mixture was filtered and the filtrate was concentrated under reduced pressure to give methyl 4-(2-aminoethyl)-1-methyl-1H-pyrazole-5-carboxylate (1.2 g, crude) as a white solid, which was used into the next step without further purification: ¹H NMR (400 MHz, DMSO-d₆) δ=7.75 (br s, 1H), 7.36 (s, 1H), 4.02 (s, 3H), 3.56 (s, 2H), 3.35 (dt, J=2.8, 6.8 Hz, 3H), 2.67 (t, J=6.8 Hz, 2H).

Step C: A mixture of methyl 4-(2-aminoethyl)-1-methyl-1H-pyrazole-5-carboxylate (1.1 g, 6.00 mmol, 1 eq.) and TEA (2.43 g, 24.02 mmol, 3.34 mL, 4 eq.) in toluene (15 mL) was degassed and purged with N₂ three times, then the mixture was stirred at 80° C. for 12 h under N₂ atmosphere. The reaction mixture was cooled and concentrated under vacuum. Then, the residue was diluted with EtOAc (100 mL×3), washed with H₂O (50 mL) and brine (50 mL). The organic layer was dried over Na₂SO₄, filtered and concentrated under vacuum to dryness to give a crude product. The residue was purified by flash silica gel chromatography (hexanes to hexanes/EtOAc=62/38) to yield 1-methyl-1,4,5,6-tetrahydro-7H-pyrazolo[3,4-c]pyridin-7-one (780 mg, 4.95 mmol, 82.50% yield) as a white solid. LCMS [M+H]⁺=152.0; ¹H NMR (400 MHz, CDCl₃) δ=7.35 (s, 1H), 5.69-5.41 (m, 1H), 4.18 (s, 3H), 3.56 (dt, J=2.8, 6.8 Hz, 2H), 2.83 (t, J=6.8 Hz, 2H).

Step D: To a solution of 1-methyl-1,4,5,6-tetrahydro-7H-pyrazolo[3,4-c]pyridin-7-one (100 mg, 661.53 μmol, 1 eq.) in DMF (5 mL) was added NBS (129.52 mg, 727.68 μmol, 1.1 eq.) at 0° C. The mixture was stirred at 25° C. for 2 h. The reaction mixture was diluted with EtOAc (30 mL), washed by water (10 mL) and brine (10 mL). The organic layer was dried over anhydrous sodium sulfate and concentrated under vacuum. The residue was purified by flash silica gel chromatography (hexanes to hexanes/EtOAc=42/58) to yield 3-bromo-1-methyl-1,4,5,6-tetrahydro-7H-pyrazolo[3,4-c]pyridin-7-one (133 mg, 0.52 mmol, 78.85% yield) as a white solid. LCMS [M+H]⁺=229.9; ¹H NMR (400 MHz, CDCl₃) δ=5.68 (br s, 1H), 4.16 (s, 3H), 3.63-3.48 (m, 2H), 2.77-2.73 (m, 2H).

Step E: A mixture of 3-bromo-1-methyl-1,4,5,6-tetrahydro-7H-pyrazolo[3,4-c]pyridin-7-one (200 mg, 869.33 μmol, 1 eq.), vinylboronic acid pinacol ester (160.67 mg, 1.04 mmol, 176.95 μL, 1.2 eq.), Pd(dppf) C12 (63.61 mg, 86.93 μmol, 0.1 eq.), K₂CO₃ (360.45 mg, 2.61 mmol, 3 eq.) in dioxane (2 mL) and H₂O (0.4 mL) was degassed and purged with N₂ three times, then the mixture was stirred at 100° C. for 16 h under N₂ atmosphere. The reaction mixture was diluted with EtOAc (20 mL×3), washed by water (15 mL) and brine (15 mL). The organic layer was dried over anhydrous sodium sulfate and concentrated under vacuum. The residue was purified by flash silica gel chromatography (hexanes to hexanes/EtOAc=38/62) to yield 1-methyl-3-vinyl-1,4,5,6-tetrahydro-7H-pyrazolo[3,4-c]pyridin-7-one (80 mg, 361.17 μmol, 41.55% yield) as a white solid. LCMS [M+H]⁺=178.0; ¹H NMR (400 MHz, CDCl₃) δ=6.71 (dd, J=11.2, 18.0 Hz, 1H), 5.73-5.62 (m, 1H), 5.50 (br s, 1H), 5.41-5.30 (m, 1H), 4.17 (s, 3H), 3.63-3.56 (m, 2H), 2.91 (t, J=6.8 Hz, 2H).

Step F: To a solution of 1-methyl-3-vinyl-1,4,5,6-tetrahydro-7H-pyrazolo[3,4-c]pyridin-7-one (140 mg, 790.06 μmol, 1 eq.) in dioxane (2 mL) and H₂O (0.8 mL) was added K₂OsO₄ (29.11 mg, 79.01 μmol, 0.1 eq.) and NaIO₄ (844.93 mg, 3.95 mmol, 218.89 μL, 5 eq.) under N₂ atmosphere at 0° C. The mixture was stirred at 25° C. for 2 h. Then, sat. aq. NaHSO₃ (3 mL) was added into the mixture, and the reaction mixture was concentrated under vacuum. The residue was diluted with EtOAc (3 mL×3), washed with H₂O (2 mL) and brine (2 mL). The combined organics were dried over Na₂SO₄, filtered and concentrated under vacuum to dryness to give a crude product. The residue was purified by flash silica gel chromatography (hexanes to hexanes/EtOAc=66/34) to yield 1-methyl-7-oxo-4,5,6,7-tetrahydro-1H-pyrazolo[3,4-c]pyridine-3-carbaldehyde (110 mg, 564.81 μmol, 71.49% yield) as a white solid. LCMS [M+H]⁺=180.0; ¹H NMR (400 MHz, CDCl₃) δ=10.02 (s, 1H), 5.72 (br s, 1H), 4.28 (s, 3H), 3.64-3.58 (m, 2H), 3.17-3.10 (m, 2H).

Step G: To a solution of 1-methyl-7-oxo-4,5,6,7-tetrahydro-1H-pyrazolo[3,4-c]pyridine-3-carbaldehyde (110 mg, 613.92 μmol, 1 eq.) in MeOH (4 mL) was added NaBH₄ (116.13 mg, 3.07 mmol, 5 eq.) at 0° C. The mixture was stirred at 25° C. for 2 h. Water (2 mL) was added into the mixture. Then, the reaction mixture was diluted with EtOAc (3 mL), washed by water (2 mL) and brine (2 mL). The organic layer was dried over anhydrous sodium sulfate and concentrated under vacuum. The residue was purified by flash silica gel chromatography (EtOAc to EtOAc/MeOH=85/15) to yield 3-(hydroxymethyl)-1-methyl-1,4,5,6-tetrahydro-7H-pyrazolo[3,4-c]pyridin-7-one (50 mg, 248.36 μmol, 40.45% yield) as a white solid. LCMS [M+H]⁺=182.0; ¹H NMR (400 MHz, DMSO-d₆) δ=7.71 (br s, 1H), 4.98 (t, J=5.6 Hz, 1H), 4.40 (d, J=5.6 Hz, 2H), 3.98 (s, 3H), 3.38-3.32 (m, 2H), 2.70 (t, J=6.8 Hz, 2H).

### Intermediate 64: Synthesis of 1,3-dimethyl-6,7-dihydroimidazo[1,5-a]pyrazin-8(5H)-one

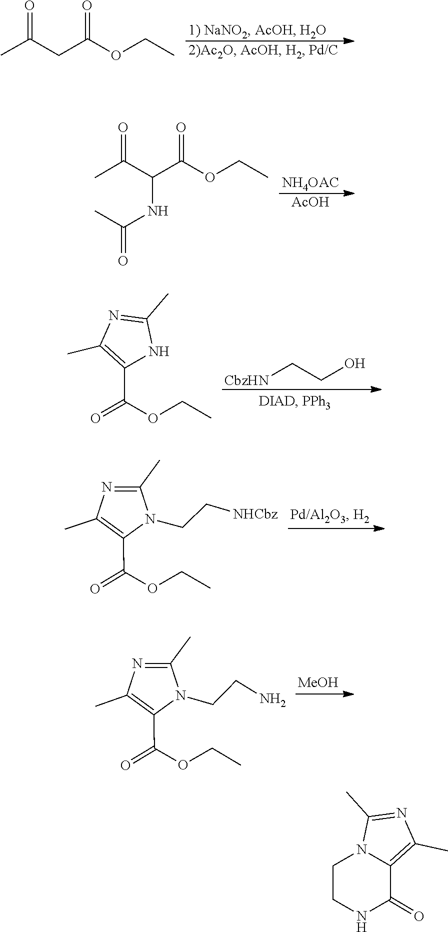

Step A: A solution of NaNO₂ (19.88 g, 288.15 mmol, 1.25 eq.) in water (200 mL) was added dropwise over 1 h to a solution of ethyl acetoacetate (30 g, 230.52 mmol, 29.18 mL, 1 eq.) in AcOH (200 mL) at 20° C. The resulting reaction mixture was stirred for another 1 h at 20° C. Then, H₂O (400 mL) was added. After stirring for another 2 h, the reaction mixture was extracted with EtOAc (800 mL×3), and successively washed with aqueous NaHCO₃ (800 mL), water (800 mL), and brine (500 mL). The organic layers were dried with Na₂SO₄ and concentrated under reduced pressure to afford a crude residue. The crude product was dissolved in a mixture of Ac₂O (29.42 g, 288.15 mmol, 27.06 mL, 1.25 eq.) and AcOH (200 mL), then Pd/C (3 g, 2.82 mmol, 10% purity, 0.1 eq.) was added portion-wise. The hydrogenator was filled with N₂ first then, replaced with hydrogen atmosphere and stirred under H₂ (50 psi) at 20° C. for 16 h. Then, the reaction mixture was concentrated under reduced pressure to afford the crude compound. The residue was purified by flash silica gel chromatography (hexanes to hexanes/EtOAc=30/70) to give ethyl 2-acetamido-3-oxobutanoate (32.2 g, 154.81 mmol, 67.16% yield) as yellow oil. LCMS [M+H]⁺=187.9; ¹H NMR (400 MHz, DMSO-d₆) δ=8.70-8.57 (m, 1H), 5.19 (d, J=7.2 Hz, 1H), 4.16 (dq, J=3.2, 7.2 Hz, 2H), 2.23 (s, 3H), 1.92 (s, 3H), 1.21 (t, J=7.2 Hz, 3H).

Step B: To a solution of ethyl 2-acetamido-3-oxobutanoate (0.5 g, 2.67 mmol, 1 eq.) in AcOH (50 mL) was added NH₄OAc (1.03 g, 13.36 mmol, 5 eq.). The mixture was stirred at 120° C. for 16 h. The reaction mixture was concentrated under reduced pressure to afford the residue, which was purified by flash silica gel chromatography (hexanes to hexanes/EtOAc=30/70) to give ethyl 2,4-dimethyl-1H-imidazole-5-carboxylate (300 mg, 856.16 μmol, 32.05% yield) as yellow oil: ¹H NMR (400 MHz, DMSO-d₆) δ=12.44-11.96 (m, 1H), 4.19 (d, J=4.4 Hz, 2H), 2.34 (s, 3H), 2.23 (s, 3H), 1.27 (t, J=7.2 Hz, 3H).

Step C: To a solution of benzyl(2-hydroxyethyl)carbamate (435.25 mg, 2.23 mmol, 1.5 eq.), ethyl 2,4-dimethyl-1H-imidazole-5-carboxylate (0.25 g, 1.49 mmol, 1 eq.), PPh₃ (779.72 mg, 2.97 mmol, 2 eq.) in THF (10 mL) was added DIAD (601.12 mg, 2.97 mmol, 576.34 μL, 2 eq.) at −60° C. The mixture was stirred at 20° C. for 16 h under N₂ atmosphere. Then, the mixture was concentrated. The residue was purified by flash silica gel chromatography (DCM to EtOAc) to give a residue. The residue was adjusted pH to 1 with 2 N HCl (20 mL), the mixture was extracted with EtOAc (30 mL×3), the aqueous phase neutralized with saturated Na₂CO₃ solution to pH=8-9. The mixture was extracted with EtOAc (20 mL×3). The combined organic layers were washed with brine (40 mL), dried over Na₂SO₄, filtered and concentrated. The residue was purified again by flash silica gel chromatography (DCM to EtOAc) to give ethyl 1-(2-(((benzyloxy)carbonyl)amino)ethyl)-2,4-dimethyl-1H-imidazole-5-carboxylate (0.07 g, 182.40 μmol, 12.27% yield) as a white solid: ¹H NMR (400 MHz, DMSO-d₆) δ=7.44 (s, 1H), 7.38-7.28 (m, 5H), 5.01 (s, 2H), 4.24-4.18 (m, 4H), 3.30-3.22 (m, 2H), 2.31 (s, 3H), 2.23 (s, 3H), 1.29 (t, J=7.2 Hz, 3H).

Step D: The solution ethyl 1-(2-(((benzyloxy)carbonyl)amino)ethyl)-2,4-dimethyl-1H-imidazole-5-carboxylate (70 mg, 202.67 μmol, 1 eq.) in MeOH (10 mL) was pumped (0.3 mL/min) to a fixed bed (completely packed with granular catalyst 5% Pd/Al₂O₃) at 50° C. under H₂ (1 MPa, the flow rate of H₂ was 30 mL/min). The mixture was concentrated to give ethyl 1-(2-aminoethyl)-2,4-dimethyl-1H-imidazole-5-carboxylate (55 mg, crude) as a white solid, which was used into the next step without further purification: ¹H NMR (400 MHz, DMSO-d₆) δ=4.22 (d, J=7.2 Hz, 2H), 4.13-4.10 (m, 2H), 3.20-3.09 (m, 2H), 2.76-2.71 (m, 2H), 2.36-2.31 (m, 6H), 1.29 (s, 3H).

Step E: A solution of ethyl 1-(2-aminoethyl)-2,4-dimethyl-1H-imidazole-5-carboxylate (1 g, 4.73 mmol, 1 eq.) in MeOH (20 mL) was stirred at 80° C. for 1 h under N₂ atmosphere. The reaction mixture was concentrated under reduced pressure to give 1,3-dimethyl-6,7-dihydroimidazo[1,5-a]pyrazin-8(5H)-one (625 mg, crude) as a white solid, which was used into the next step without further purification: ¹H NMR (400 MHz, DMSO-d₆) δ=7.67 (s, 1H), 3.97 (t, J=5.6 Hz, 2H), 3.43 (s, 2H), 2.33-2.22 (m, 6H).

### Intermediate 65: Synthesis of 2-bromo-4-((5-(difluoromethyl)pyridin-3-yl)oxy)pyridine

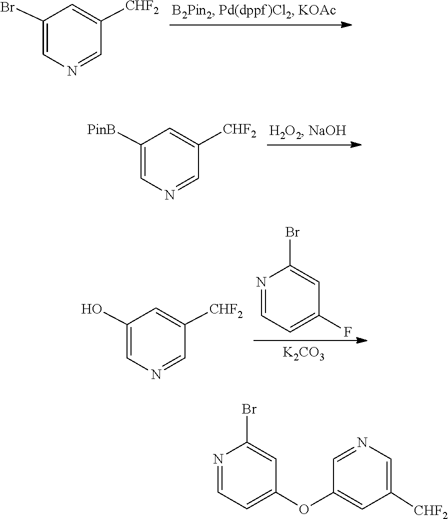

Step A: To a mixture of 3-bromo-5-(difluoromethyl)pyridine (14 g, 67.31 mmol, 1 eq.) in dioxane (400 mL) were added B₂Pin₂ (34.18 g, 134.61 mmol, 2 eq.), KOAc (19.82 g, 201.92 mmol, 3 eq.) and Pd(dppf)Cl₂ (2.46 g, 3.37 mmol, 0.05 eq.) at 25° C. The mixture was stirred at 80° C. for 14 h under N₂. The reaction mixture was cooled and concentrated under reduced pressure to give the residue, which was diluted with H₂O (400 mL) and extracted with EtOAc (300 mL×3). The combined organic layers were washed with brine (100 mL×2), dried over Na₂SO₄, filtered and concentrated under reduced pressure to give a residue, which was purified by flash silica gel chromatography (hexanes to hexanes/EtOAc=50/50) to give 3-(difluoromethyl)-5-(4,4,5,5-tetramethyl-1,3,2-dioxaborolan-2-yl)pyridine (16.2 g, 63.51 mmol, 94.36% yield) as black oil: ¹H NMR (500 MHz, DMSO-d₆) δ=8.90 (s, 1H), 8.16 (d, J=0.5 Hz, 1H), 7.93 (s, 1H), 7.17 (t, J=55.5 Hz, 1H), 1.33 (s, 12H).

Step B: To a mixture of 3-(difluoromethyl)-5-(4,4,5,5-tetramethyl-1,3,2-dioxaborolan-2-yl)pyridine (13.2 g, 51.75 mmol, 1 eq.) in THF (140 mL) were added H₂O₂ (27.97 g, 246.69 mmol, 23.70 mL, 30% purity, 4.77 eq.) and NaOH (2.07 g, 51.75 mmol, 1 eq.) in H₂O (70 mL) at 0° C. The mixture was stirred at 0° C. for 1 h under N₂. The reaction mixture was quenched by addition sat. Na₂SO₃ (250 mL) and the mixture was stirred at 0° C. for 0.5 h. Then diluted with H₂O (100 mL) and extracted with EtOAc (150 mL×3), the combined organic layers were concentrated under reduced pressure to give 5-(difluoromethyl)pyridin-3-ol (6.2 g, crude) as brown oil, which was used in the next step without further purification: ¹H NMR (400 MHz, DMSO-d₆) δ=10.39 (s, 1H), 8.29-8.28 (m, 1H), 8.23 (s, 1H), 7.30 (s, 1H), 7.07 (t, J=55.2 Hz, 1H).

Step C: To a mixture of 5-(difluoromethyl)pyridin-3-ol (6.2 g, 42.73 mmol, 1 eq.) in MeCN (150 mL) was added 2-bromo-4-fluoropyridine (8.27 g, 47.00 mmol, 1.1 eq.) and K₂CO₃ (17.72 g, 128.18 mmol, 3 eq.) at 25° C. The mixture was stirred at 80° C. for 16 h. The reaction mixture was concentrated. The residue was diluted with H₂O (200 mL) and extracted with EtOAc (200 mL×3). The combined organic layers were washed with brine (100 mL×2), dried over Na₂SO₄, filtered and concentrated under reduced pressure to give a residue. The residue was purified by flash silica gel chromatography (hexanes to hexanes/EtOAc=70/30) to give 2-bromo-4-((5-(difluoromethyl)pyridin-3-yl)oxy)pyridine (4.77 g, 15.84 mmol, 37.08% yield) as a white solid: ¹H NMR (400 MHz, DMSO-d₆) δ=8.91-8.64 (m, 2H), 8.33 (d, J=5.6 Hz, 1H), 7.98 (s, 1H), 7.34 (d, J=2.0 Hz, 1H), 7.33-7.03 (m, 2H).

### Intermediate 66: Synthesis of 1,3-dimethyl-1,5-dihydro-4H-pyrazolo[4,3-c]pyridin-4-one

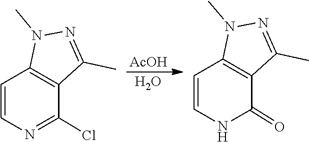

Step A: To a solution of 4-chloro-1,3-dimethyl-1H-pyrazolo[4,3-c]pyridine (168 mg, 925.00 μmol, 1 eq.) in AcOH (2.10 g, 34.94 mmol, 2 mL, 37.77 eq.) and H₂O (0.4 mL) was stirred at 100° C. for 16 h. The reaction mixture was concentrated and then water (20 mL) was added. The mixture was extracted with EtOAc (20 mL×3). The combined organic layers were washed with brine (40 mL) and dried over Na₂SO and filtered. The filtrate was concentrated under reduced pressure to give a residue, which was purified by flash silica gel chromatography (DCM to DCM/MeOH=20/1, TLC: DCM/MeOH=10/1, R_{f}=0.42) to give 1,3-dimethyl-1,5-dihydro-4H-pyrazolo[4,3-c]pyridin-4-one (138 mg, 845.71 μmol, 91.43% yield) as a white solid: ¹H NMR (400 MHz, CDCl₃) δ=10.11 (br s, 1H), 7.15-7.07 (m, 1H), 6.29 (d, J=7.2 Hz, 1H), 3.89 (s, 3H), 2.65 (s, 3H).

### Intermediate 67: Synthesis of 1-(2-(benzyloxy)ethyl)-3-methyl-1,5-dihydro-4H-pyrazolo[4,3-c]pyridin-4-one

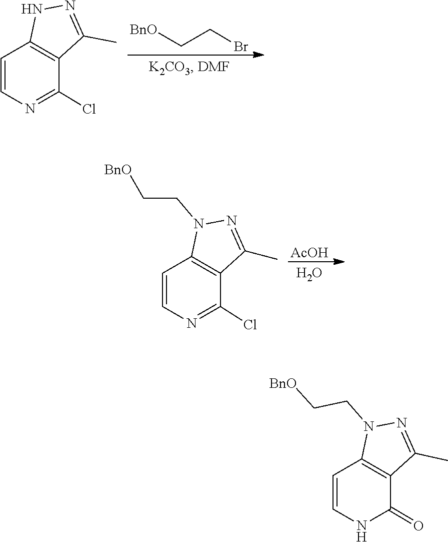

Step A: To a solution of 4-chloro-3-methyl-1H-pyrazolo[4,3-c]pyridine (300 mg, 1.79 mmol, 1 eq.) in DMF (3 mL) was added K₂CO₃ (494.78 mg, 3.58 mmol, 2 eq.) and ((2-bromoethoxy)methyl) benzene (462.01 mg, 2.15 mmol, 339.72 μL, 1.2 eq.). The mixture was stirred at 65° C. for 3 h. The reaction mixture was concentrated and then water (20 mL) was added. The mixture was extracted with EtOAc (20 mL×3). The combined organic layers were washed with brine (30 mL) and dried over Na₂SO₄ and filtered. The filtrate was concentrated under reduced pressure to give a residue, which was purified by column chromatography (hexanes to hexanes/EtOAc=3/2, TLC: hexane/EtOAc=1/1, R_{f}=0.66) to give 1-(2-(benzyloxy)ethyl)-4-chloro-3-methyl-1H-pyrazolo[4,3-c]pyridine (283 mg, 937.80 μmol, 52.39% yield) as colorless oil: ¹H NMR (400 MHz, CDCl₃) δ=8.07 (d, J=6.0 Hz, 1H), 7.26-7.22 (m, 4H), 7.10-7.06 (m, 2H), 4.46 (t, J=5.2 Hz, 2H), 4.41 (s, 2H), 3.86 (t, J=5.2 Hz, 2H), 2.74 (s, 3H).

Step B: A solution of 1-(2-(benzyloxy)ethyl)-4-chloro-3-methyl-1H-pyrazolo[4,3-c]pyridine (283 mg, 937.80 μmol, 1 eq.) in HOAc (3.15 g, 52.40 mmol, 3 mL, 55.88 eq.) and H₂O (0.6 mL) was stirred at 100° C. for 16 h. The mixture was concentrated under reduced pressure. The residue was purified by column chromatography (hexanes to EtOAc, TLC: hexane/EtOAc=0/1, R_{f}=0.06) to give 1-(2-(benzyloxy)ethyl)-3-methyl-1,5-dihydro-4H-pyrazolo[4,3-c]pyridin-4-one (178 mg, 628.26 μmol, 66.99% yield) as a white solid: ¹H NMR (400 MHz, CDCl₃) δ=9.80-9.30 (m, 1H), 7.31-7.28 (m, 1H), 7.26-7.20 (m, 2H), 7.17-7.12 (m, 2H), 7.08-7.01 (m, 1H), 6.39 (d, J=7.2 Hz, 1H), 4.44 (s, 2H), 4.34 (t, J=5.2 Hz, 2H), 3.85 (t, J=5.2 Hz, 2H), 2.65 (s, 3H).

### Intermediate 68: Synthesis of 1-(2-hydroxyethyl)-3-methyl-1,5-dihydro-4H-pyrazolo[3,4-d]pyrimidin-4-one

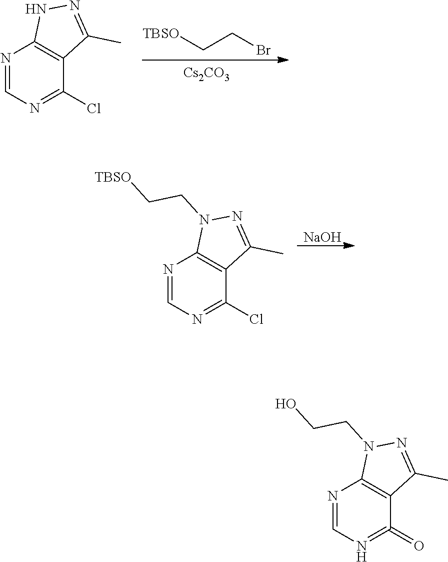

Step A: To a solution of 4-chloro-3-methyl-1H-pyrazolo[3,4-d]pyrimidine (1.04 g, 6.17 mmol, 1 eq.) and (2-bromoethoxy) (tert-butyl)dimethylsilane (2.21 g, 9.25 mmol, 1.5 eq.) in DMF (50 mL) was added Cs₂CO₃ (4.02 g, 12.34 mmol, 2 eq.) at 25° C. The mixture was stirred at 40° C. for 1 h. The mixture was poured into H₂O (100 mL) and extracted with EtOAc (50 mL×3). The combined organic layers were washed with brine (100 mL), dried over Na₂SO₄, filtered and concentrated to give a residue, which was purified by flash silica gel chromatography (hexanes to hexanes/EtOAc=95/5, TLC: hexanes/EtOAc=3/1, R_{f}=0.5) to give 1-(2-((tert-butyldimethylsilyl)oxy)ethyl)-4-chloro-3-methyl-1H-pyrazolo[3,4-d]pyrimidine (655 mg, 1.80 mmol, 29.23% yield) as yellow oil. LCMS [M+H]⁺=327.1; ¹H NMR (400 MHz, DMSO-d₆) δ=8.77 (s, 1H), 4.48 (t, J=5.2 Hz, 2H), 3.99 (t, J=5.2 Hz, 2H), 2.64 (s, 3H), 0.61 (s, 9H), 0.25-0.00 (m, 6H).

Step B: To a solution of 1-(2-((tert-butyldimethylsilyl)oxy)ethyl)-4-chloro-3-methyl-1H-pyrazolo[3,4-d]pyrimidine (635 mg, 1.94 mmol, 1 eq.) in dioxane (4 mL) was added NaOH (2 M, 3.89 mL, 4 eq.) at 25° C. The mixture was stirred at 100° C. for 3 h. The reaction mixture was cooled and concentrated under reduced pressure to remove dioxane. The residue was diluted with water (8 mL) and adjusted pH to 3-4 with 2 N HCl. The precipitates were filtered, and the filter cake was collected to give 1-(2-hydroxyethyl)-3-methyl-1,5-dihydro-4H-pyrazolo[3,4-d]pyrimidin-4-one (185 mg, crude) as a white solid. LCMS [M+H]⁺=195.0; ¹H NMR (400 MHz, DMSO-d₆) δ=12.00 (br s, 1H), 7.99 (d, J=4.2 Hz, 1H), 4.21 (t, J=6.0 Hz, 2H), 3.76 (t, J=6.0 Hz, 2H), 2.42 (s, 3H).

### Intermediate 69: Synthesis of tert-butyl 6-hydroxy-2,3-dihydro-4H-pyrido[3,2-b][1,4]oxazine-4-carboxylate

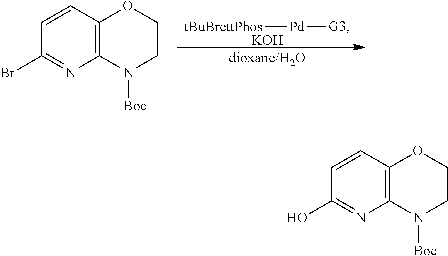

Step A: A mixture of tert-butyl 6-bromo-2,3-dihydro-4H-pyrido[3,2-b][1,4]oxazine-4-carboxylate (500 mg, 1.59 mmol, 1 eq.), KOH (178.02 mg, 3.17 mmol, 187.39 μL, 2 eq.), tBuBrettPhos-Pd-G3 ([(2-Di-tert-butylphosphino-3,6-dimethoxy-2′,4′,6′-triisopropyl-1,1′-biphenyl)-2-(2′-amino-1,1′-biphenyl)]palladium (II) methanesulfonate, CAS #1536473-72-9, 135.55 mg, 158.65 μmol, 0.1 eq.) in dioxane (7.5 mL) and H₂O (2.5 mL) was degassed and purged with N₂ three times, and then the mixture was stirred at 70° C. for 1 h under N₂ atmosphere. The reaction mixture was diluted with H₂O (20 mL) and extracted with EtOAc (60 mL). The combined organic layers were dried over Na₂SO₄ filtered and concentrated under reduced pressure to give a residue. The residue was purified by flash silica gel chromatography (hexanes to hexanes/EtOAc=3/7) to yield tert-butyl 6-hydroxy-2,3-dihydro-4H-pyrido[3,2-b][1,4]oxazine-4-carboxylate (390 mg, 1.55 mmol, 97.45% yield) as a brown solid: ¹H NMR (400 MHz, CDCl₃) δ=11.48 (br s, 1H), 7.14 (d, J=9.6 Hz, 1H), 6.16 (d, J=9.6 Hz, 1H), 4.16-4.09 (m, 2H), 3.88-3.79 (m, 2H), 1.54 (s, 9H).

### Intermediate 70: Synthesis of 3-(hydroxymethyl)-1-methyl-6,7-dihydroimidazo[1,5-a]pyrazin-8(5H)-one

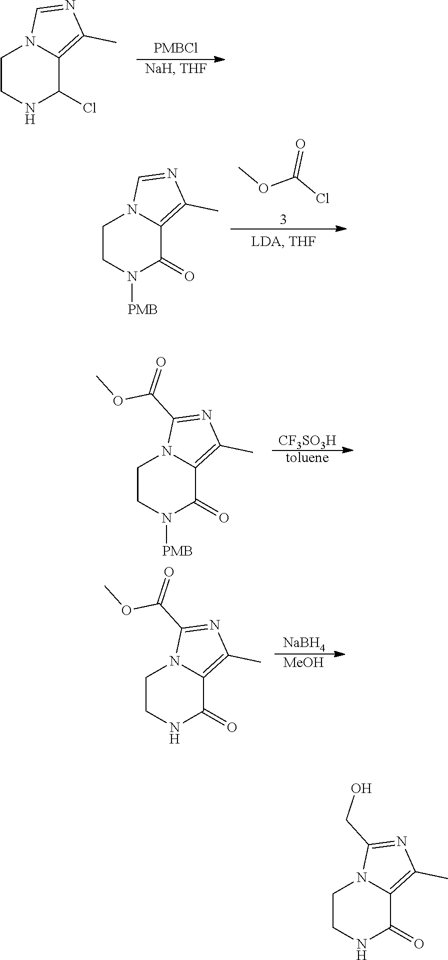

Step A: To a solution of 1-methyl-6,7-dihydroimidazo[1,5-a]pyrazin-8(5H)-one (940 mg, 6.22 mmol, 1 eq.) in THF (10 mL) was added NaH (358.18 mg, 7.46 mmol, 50% purity, 1.2 eq.), the mixture was stirred at 0° C. for 15 min. Then PMB-C1 (1.27 g, 8.08 mmol, 1.10 mL, 1.3 eq.) was added in one portion. The mixture was stirred at 25° C. for 45 min. Then, the reaction mixture was quenched dropwise with 1 mL of water. The reaction mixture was concentrated under reduced pressure then diluted with EtOAc (300 mL) and washed with water (100 mL). The organic layer was dried over sodium sulfate, filtered and concentrated under reduced pressure to give a residue. The residue was purified by column chromatography (DCM to DCM/MeOH=10/1) to give 7-(4-methoxybenzyl)-1-methyl-6,7-dihydroimidazo[1,5-a]pyrazin-8(5H)-one (1.6 g, 5.90 mmol, 94.84% yield) as a white solid: ¹H NMR (400 MHz, CDCl₃) δ=7.39 (s, 1H), 7.25 (d, J=8.6 Hz, 2H), 6.92-6.80 (m, 2H), 4.68 (s, 2H), 4.11-4.01 (m, 2H), 3.81 (s, 3H), 3.55-3.49 (m, 2H), 2.59 (s, 3H).

Step B: To a solution of 7-(4-methoxybenzyl)-1-methyl-6,7-dihydroimidazo[1,5-a]pyrazin-8(5H)-one (560 mg, 2.06 mmol, 1 eq.) was stirred in THF (5 mL) at −78° C. Then, LDA (2.0 M, 1.34 mL, 1.3 eq.) was added dropwise via a 2.5 mL syringe, the reaction was continue stirring at −78° C. After 15 min, methyl chloroformate (780.18 mg, 8.26 mmol, 637.92 μL, 4 eq.) was added dropwise. The reaction mixture was warmed up to 25° C. and stirred for 45 min. 1 mL of water was added dropwise to the reaction to quench the reaction. Then the mixture was diluted with EtOAc (100 mL) and extracted. The organic layer was dried over sodium sulfate, filtered and concentrated under reduced pressure to give a residue.

The residue was purified by column chromatography (hexanes to hexanes/EtOAc=1/10) to give methyl 7-(4-methoxybenzyl)-1-methyl-8-oxo-5,6,7,8-tetrahydroimidazo[1,5-a]pyrazine-3-carboxylate (230 mg, 698.35 μmol, 33.83% yield) as a white solid: ¹H NMR (400 MHz, CDCl₃) δ=7.18 (d, J=8.8 Hz, 2H), 6.81 (d, J=8.6 Hz, 2H), 4.65-4.58 (m, 2H), 4.54-4.45 (m, 2H), 3.88 (s, 3H), 3.74 (s, 3H), 3.53-3.45 (m, 2H), 2.55 (s, 3H).

Step C: To a solution of methyl 7-(4-methoxybenzyl)-1-methyl-8-oxo-5,6,7,8-tetrahydroimidazo[1,5-a]pyrazine-3-carboxylate (230 mg, 698.35 μmol, 1 eq.) in toluene (10 mL) at 0° C. was added TfOH (1.05 g, 6.98 mmol, 617.96 μL, 10 eq.). The mixture was warmed to 25° C. and stirred for 0.5 h. The reaction mixture was charged with MeOH (10 mL) then concentrated under reduced pressure to give a residue. The residue was purified by column chromatography (DCM to DCM/MeOH=10/1 (1% NH₃H₂O)) to give methyl 1-methyl-8-oxo-5,6,7,8-tetrahydroimidazo[1,5-a]pyrazine-3-carboxylate (145 mg, 693.11 μmol, 99.25% yield) as a white solid: ¹H NMR (400 MHz, DMSO-d₆) δ=8.17 (br s, 1H), 4.59-4.47 (m, 2H), 3.87 (s, 3H), 3.56-3.41 (m, 2H), 2.40 (s, 3H).

Step D: To a solution of methyl 1-methyl-8-oxo-5,6,7,8-tetrahydroimidazo[1,5-a]pyrazine-3-carboxylate (145 mg, 693.11 μmol, 1 eq.) in MeOH (5 mL) was added NaBH₄ (524.44 mg, 13.86 mmol, 20 eq.). The mixture was stirred at 25° C. for 1 h. Then, the reaction mixture was concentrated and diluted with DCM (20 mL). The organic phase was washed with water (10 mL), dried over sodium sulfate, filtered and concentrated under reduced pressure to give a residue. The residue was purified by column chromatography (DCM to DCM/MeOH=10/1 (1% NH₃H₂O)) to give 3-(hydroxymethyl)-1-methyl-6,7-dihydroimidazo[1,5-a]pyrazin-8(5H)-one (125 mg, 689.88 μmol, 99.53% yield) as a white solid: ¹H NMR (400 MHz, DMSO-d₆) δ=7.73 (br s, 1H), 5.45-5.28 (m, 1H), 4.50-4.42 (m, 2H), 4.09 (br d, J=5.6 Hz, 2H), 3.50-3.39 (m, 2H), 2.31 (s, 3H).

### Intermediate 71: Synthesis of tert-butyl 7-((2-bromopyridin-4-yl)methyl)-2,3-dihydro-1H-pyrido[2,3-b][1,4]oxazine-1-carboxylate

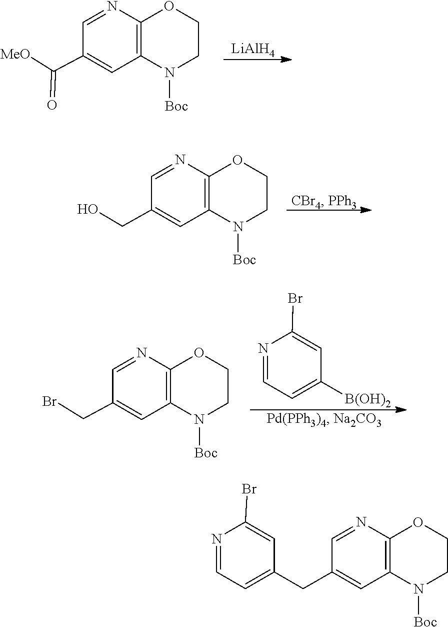

Step A: To a solution of 1-(tert-butyl) 7-methyl 2,3-dihydro-1H-pyrido[2,3-b][1,4]oxazine-1,7-dicarboxylate (0.8 g, 2.72 mmol, 1 eq.) in THF (15 mL) was added LiAlH₄ (2.5 M, 3.26 mL, 3 eq.) at −20° C. The mixture was stirred at −20° C. for 15 min under N₂ atmosphere. The reaction mixture was quenched by the addition of saturated aqueous NH₄Cl solution (8 mL) at −10° C. The reaction mixture was concentrated under reduced pressure to give a residue. The residue was purified by flash silica gel chromatography (hexanes to EtOAc) to give tert-butyl 7-(hydroxymethyl)-2,3-dihydro-1H-pyrido[2,3-b][1,4]oxazine-1-carboxylate (0.58 g, 2.18 mmol, 80.13% yield) as a white solid. ¹H NMR (400 MHz, DMSO-d₆) δ=8.21 (s, 1H), 7.77 (d, J=1.6 Hz, 1H), 5.21 (t, J=5.6 Hz, 1H), 4.42 (d, J=5.6 Hz, 2H), 4.35-4.27 (m, 2H), 3.86-3.76 (m, 2H), 1.49 (s, 9H).

Step B: To a solution of CBr₄ (1.12 g, 3.38 mmol, 2 eq.), PPh₃ (886.46 mg, 3.38 mmol, 2 eq.) in THF (15 mL) was added tert-butyl 7-(hydroxymethyl)-2,3-dihydro-1H-pyrido[2,3-b][1,4]oxazine-1-carboxylate (0.45 g, 1.69 mmol, 1 eq.) at 20° C. The mixture was stirred at 20° C. for 1 h under N₂ atmosphere. The reaction mixture was concentrated under reduced pressure to give a residue. The residue was purified by flash silica gel chromatography (hexanes to hexanes/EtOAc=30/70) to give tert-butyl 7-(bromomethyl)-2,3-dihydro-1H-pyrido[2,3-b][1,4]oxazine-1-carboxylate (0.38 g, 1.15 mmol, 68.31% yield) as colorless oil: ¹H NMR (400 MHz, DMSO-d₆) δ=8.32 (d, J=1.6 Hz, 1H), 7.95 (d, J=2.0 Hz, 1H), 4.73 (s, 2H), 4.37-4.33 (m, 2H), 3.83-3.79 (m, 2H), 1.52 (s, 9H).

Step C: To a solution of tert-butyl 7-(bromomethyl)-2,3-dihydro-1H-pyrido[2,3-b][1,4]oxazine-1-carboxylate (0.38 g, 1.15 mmol, 1 eq.), Pd(PPh₃)₄ (133.39 mg, 115.44 μmol, 0.1 eq.) in toluene (10 mL) and EtOH (5 mL) were added (2-bromopyridin-4-yl) boronic acid (302.85 mg, 1.50 mmol, 1.3 eq.) and Na₂CO₃ (367.05 mg, 3.46 mmol, 3 eq.) at 20° C. The mixture was stirred at 85° C. for 2 h under N₂ atmosphere. The reaction mixture was concentrated under reduced pressure to give a residue. The residue was purified by flash silica gel chromatography (hexanes to hexanes/EtOAc) to give tert-butyl 7-((2-bromopyridin-4-yl)methyl)-2,3-dihydro-1H-pyrido[2,3-b][1,4]oxazine-1-carboxylate (100 mg, 221.53 μmol, 19.19% yield) as a pale-yellow solid. ¹H NMR (400 MHz, DMSO-d₆) δ=8.28 (d, J=5.2 Hz, 1H), 8.00 (s, 1H), 7.86 (d, J=2.0 Hz, 1H), 7.56 (d, J=0.8 Hz, 1H), 7.31 (dd, J=1.6, 5.2 Hz, 1H), 4.35-4.28 (m, 2H), 3.94 (s, 2H), 3.81-3.75 (m, 2H), 1.42 (s, 9H).

### Intermediate 72: Synthesis of 7-((2-bromopyridin-4-yl)oxy)-2-methoxyquinoline

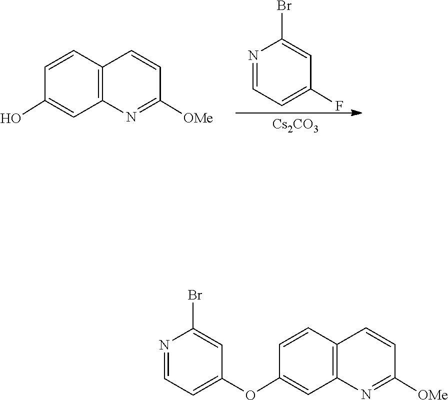

Step A: To a solution of 2-methoxyquinolin-7-ol (500 mg, 2.85 mmol, 1 eq.) in DMF (8 mL) was added 2-bromo-4-fluoropyridine (502.29 mg, 2.85 mmol, 1 eq.), then Cs₂CO₃ (1.86 g, 5.71 mmol, 2 eq.) was added. The mixture was stirred at 80° C. for 1 h. The reaction mixture was cooled and diluted with EtOAc (200 mL) and washed with water (100 mL). The organic layer was dried over sodium sulfate, filtered and concentrated under reduced pressure to give a residue. The residue was purified by column chromatography (hexanes to hexanes/EtOAc=2/1) to give 7-((2-bromopyridin-4-yl)oxy)-2-methoxyquinoline (880 mg, 2.66 mmol, 93.10% yield) as a white solid. ¹H NMR (400 MHz, CDCl₃) δ=8.27 (d, J=5.6 Hz, 1H), 8.02 (d, J=8.8 Hz, 1H), 7.81 (d, J=8.6 Hz, 1H), 7.55 (d, J=2.4 Hz, 1H), 7.15 (dd, J=2.4, 8.6 Hz, 1H), 7.08 (d, J=2.0 Hz, 1H), 6.99-6.89 (m, 2H), 4.11-3.98 (m, 3H).

### Intermediate 73: Synthesis of methyl 1-ethyl-7-oxo-6,7-dihydro-1H-pyrazolo[3,4-c]pyridine-3-carboxylate

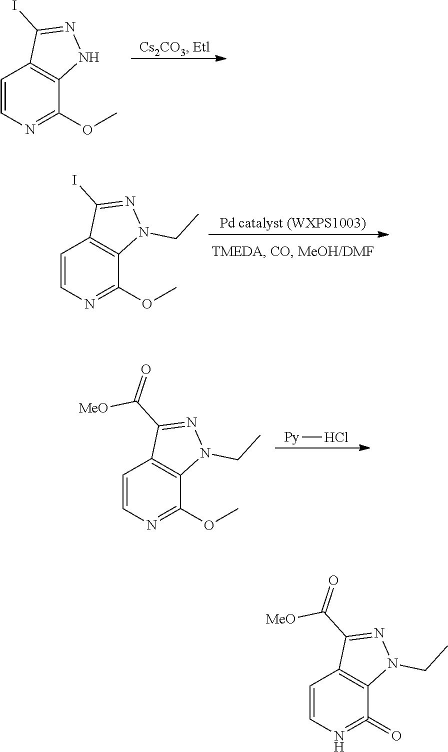

Step A: To a solution of 3-iodo-7-methoxy-1H-pyrazolo[3,4-c]pyridine (450 mg, 1.64 mmol, 1 eq.) and Cs₂CO₃ (1.07 g, 3.27 mmol, 2 eq.) in DMF (6 mL) was added EtI (306.21 mg, 1.96 mmol, 157.03 μL, 1.2 eq.) at 0° C. The mixture was stirred at 0° C. for 1 h. he reaction mixture was quenched by addition H₂O (30 mL), and extracted with EtOAc (30 mL×3). The combined organic layers were washed with brine 60 mL), dried over Na₂SO₄, filtered and concentrated under reduced pressure to give a residue. The residue was purified by column chromatography (hexanes to hexanes/EtOAc=90/10; TLC: hexanes/EtOAc=3/1, R_{f1}=0.71) to yield 1-ethyl-3-iodo-7-methoxy-1H-pyrazolo[3,4-c]pyridine (340 mg, 1.09 mmol, 66.51% yield) as a white solid. LCMS [M+H]⁺=303.9. ¹H NMR (400 MHz, DMSO-d₆) δ=7.76 (d, J=6.0 Hz, 1H), 6.98 (d, J=6.0 Hz, 1H), 4.62 (q, J=7.5 Hz, 2H), 4.07 (s, 3H), 1.40 (t, J=7.0 Hz, 3H).

Step B: 1-ethyl-3-iodo-7-methoxy-1H-pyrazolo[3,4-c]pyridine (400 mg, 1.32 mmol, 1 eq.) and TMEDA (460.07 mg, 3.96 mmol, 597.49 μL, 3 eq.) in MeOH (24 mL) and DMF (12 mL). The fixed bed (volume 50 mL) was completely packed with granular catalyst POL-Pd (WXPS1003) (WuXi LabNetwork, Shanghai, China; see also CN app. no. 2025109971019). The CO back pressure regulator was adjusted to 3.0 MPa, and the flow rate of CO was 15 mL/min. Then the solution was pumped {3 mL/min} to fixed bed {36 mL, 160° C.}. The solution was flowed through the reactor zone, then the reaction mixture was collected from the reactor output. The mixture was concentrated under vacuum to yield a residue. The residue was diluted with water (50 mL) and extracted with EtOAc (50 mL×3). The combined organic phase was washed with brine (50 mL), dried over anhydrous Na₂SO₄, filtered and concentrated under vacuum to yield a residue, which was purified by flash silica gel chromatography (hexane to hexane/EtOAc=80/20, TLC: hexane/EtOAc=3/1, R_{f}=0.50) to yield methyl 1-ethyl-7-methoxy-1H-pyrazolo[3,4-c]pyridine-3-carboxylate (130 mg, 552.63 μmol, 41.88% yield) as a yellow solid. ¹H NMR (400 MHz, DMSO-d₆) δ=7.88 (d, J=5.6 Hz, 1H), 7.54 (d, J=5.6 Hz, 1H), 4.70 (q, J=7.2 Hz, 2H), 4.10 (s, 3H), 3.92 (s, 3H), 1.45 (t, J=7.2 Hz, 3H).

Step C: To a solution of methyl 1-ethyl-7-methoxy-1H-pyrazolo[3,4-c]pyridine-3-carboxylate (150 mg, 637.65 μmol, 1 eq.) in DMF (15 mL) was added Py·HCl (1.47 g, 12.75 mmol, 20 eq.). The mixture was stirred at 80° C. for 1 h. The reaction mixture was cooled and quenched by addition of saturated Na₂CO₃ (20 mL) and extracted with EtOAc (30 mL×3). The combined organic layers were washed with brine (30 mL), dried over Na₂SO₄, filtered and concentrated under reduced pressure to give methyl 1-ethyl-7-oxo-6,7-dihydro-1H-pyrazolo[3,4-c]pyridine-3-carboxylate (130 mg, crude) as a white solid, which was used directly in the next step without further purification. LCMS [M+H]⁺=222.2. ¹H NMR (400 MHz, DMSO-d₆) δ=11.57 (s, 1H), 7.10 (d, J=4.4 Hz, 1H), 6.76 (d, J=6.8 Hz, 1H), 4.73 (q, J=7.2 Hz, 2H), 3.88 (s, 3H), 1.41 (t, J=7.2 Hz, 3H).

Intermediate 74: Synthesis of methyl 1-methyl-7-oxo-6,7-dihydro-1H-pyrazolo[3,4-c]pyridine-3-carboxylate

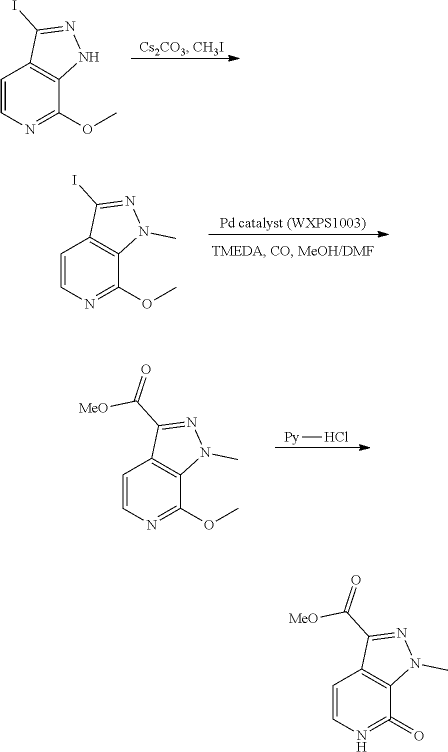

Step A: To a solution of 3-iodo-7-methoxy-1H-pyrazolo[3,4-c]pyridine (800 mg, 2.62 mmol, 1 eq.) in DMF (15 mL) was added Cs₂CO₃ (2.56 g, 7.85 mmol, 3 eq.) and MeI (445.88 mg, 3.14 mmol, 195.56 μL, 1.2 eq.) at 0° C. under N₂ atmosphere. The mixture was stirred at 0° C. for 1 h under N₂ atmosphere. The reaction mixture was quenched by addition H₂O (20 mL) and extracted with EtOAc (30 mL×3). The combined organic layers were washed with brine (30 mL), dried over Na₂SO₄, filtered and concentrated under reduced pressure to give a residue. The residue was purified by flash silica gel chromatography hexanes to hexanes/EtOAc=86/14, TLC: hexane/EtOAc=3/1, R_{f}=0.60) to yield 3-iodo-7-methoxy-1-methyl-1H-pyrazolo[3,4-c]pyridine (620 mg, 2.14 mmol, 81.93% yield) as a white solid. ¹H NMR (400 MHz, CDCl₃) δ=7.75 (d, J=5.6 Hz, 1H), 6.92 (d, J=6.0 Hz, 1H), 4.33 (s, 3H), 4.12 (s, 3H).

Step B: 3-iodo-7-methoxy-1-methyl-1H-pyrazolo[3,4-c]pyridine was subjected identically to Intermediate 73, step B to provide methyl 7-methoxy-1-methyl-1H-pyrazolo[3,4-c]pyridine-3-carboxylate (155 mg, 686.67 μmol, 90.23% yield) as a white solid. ¹H NMR (400 MHz, CDCl₃) δ=7.85 (d, J=5.6 Hz, 1H), 7.60 (d, J=6.0 Hz, 1H), 4.40 (s, 3H), 4.14 (s, 3H), 4.03 (s, 3H).

Step C: methyl 7-methoxy-1-methyl-1H-pyrazolo[3,4-c]pyridine-3-carboxylate was subjected identically to Intermediate 73, step C to provide methyl 1-methyl-7-oxo-6,7-dihydro-1H-pyrazolo[3,4-c]pyridine-3-carboxylate (270 mg, crude) as a white solid, which was used in the next step without further purification. ¹H NMR (400 MHz, CDCl₃) δ=10.24 (s, 1H), 7.03 (d, J=2.4 Hz, 2H), 4.47 (s, 3H), 4.01 (s, 3H).

### Intermediate 75: Synthesis of 3-(((tert-butyldimethylsilyl)oxy)methyl)-1-methoxy-6,7-dihydroimidazo[1,5-a]pyrazin-8(5H)-one

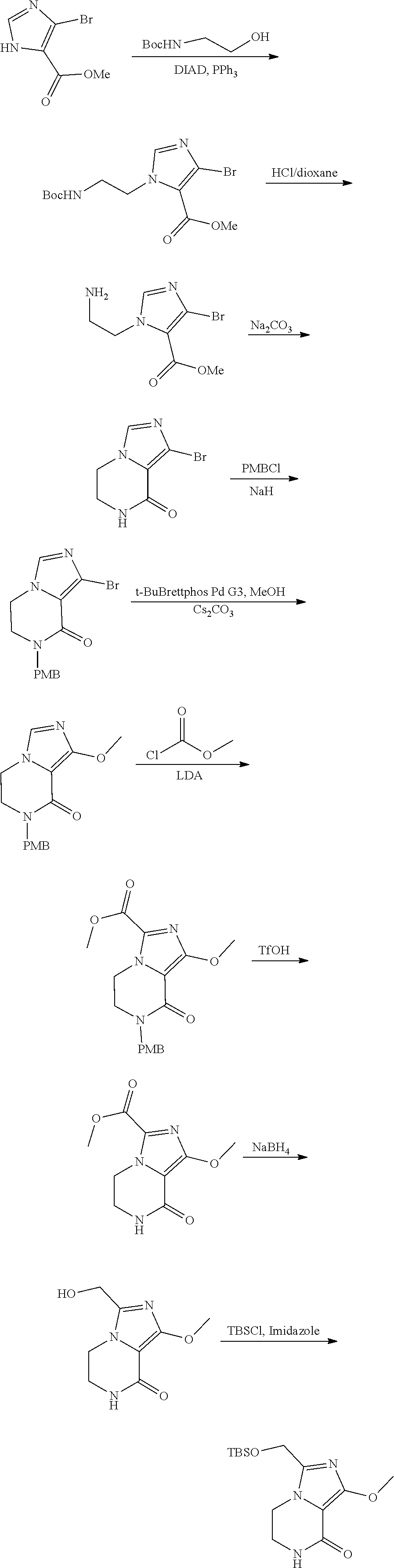

Step A: To a solution of methyl 4-bromo-1H-imidazole-5-carboxylate (1.9 g, 9.27 mmol, 1 eq.) and tert-butyl(2-hydroxyethyl)carbamate (1.94 g, 12.05 mmol, 1.86 mL, 1.3 eq.) in THF (80 mL) was added PPh₃ (3.65 g, 13.90 mmol, 1.5 eq.) and DIAD (2.81 g, 13.90 mmol, 2.70 mL, 1.5 eq.) at −60° C. under N₂. The mixture was stirred at 20° C. for 2 h under N₂. The reaction mixture was concentrated and then diluted with H₂O (100 mL) and extracted with EtOAc (100 mL×2). The combined organic layers were washed with brine (100 mL), dried over Na₂SO₄, filtered and concentrated under reduced pressure to give a residue. The residue was purified by flash silica gel chromatography (hexanes to hexanes/EtOAc=50/50) to yield methyl 4-bromo-1-(2-((tert-butoxycarbonyl)amino)ethyl)-1H-imidazole-5-carboxylate (4.3 g, 7.41 mmol, 79.95% yield) as colorless oil. LCMS [M+H]⁺=348.1.

Step B: To a solution of methyl 4-bromo-1-(2-((tert-butoxycarbonyl)amino)ethyl)-1H-imidazole-5-carboxylate (4.2 g, 7.24 mmol, 1 eq.) in DCM (20 mL) was added HCl/dioxane (2 M, 20 mL, 5.53 eq.). The mixture was stirred at 20° C. for 12 h. The mixture was concentrated to give methyl 1-(2-aminoethyl)-4-bromo-1H-imidazole-5-carboxylate (4 g, crude, HCl salt) as a white solid, which was used to next step directly. LCMS [M+H]⁺=248.0.

Step C: To a solution of methyl 1-(2-aminoethyl)-4-bromo-1H-imidazole-5-carboxylate (5.5 g, 19.33 mmol, 1 eq, HCl) in MeOH (100 mL) was added Na₂CO₃ (10.24 g, 96.65 mmol, 5 eq.). The mixture was stirred at 60° C. for 2 h. The reaction mixture was filtered and the filtrate was concentrated under reduced pressure to give a residue. The residue was purified by flash silica gel chromatography (DCM to DCM/MeOH=90/10, TLC: DCM/MeOH=10/1, R_{f}=0.6) to give 1-bromo-6,7-dihydroimidazo[1,5-a]pyrazin-8(5H)-one (3.1 g, 14.35 mmol, 74.24% yield) as a white solid. LCMS [M+H]⁺=215.9. ¹H NMR (400 MHz, DMSO-d₆) δ=7.99 (br s, 1H), 7.84 (s, 1H), 4.28-4.08 (m, 2H), 3.53-3.44 (m, 2H).

Step D: To 1-bromo-6,7-dihydroimidazo[1,5-a]pyrazin-8(5H)-one (4 g, 18.52 mmol, 1 eq.) in DMF (40 mL) was added NaH (1.11 g, 27.77 mmol, 60% purity, 1.5 eq.) at 0° C. under N₂ atmosphere. The mixture was stirred at 0° C. for 0.5 h. Then PMB-C1 (4.35 g, 27.77 mmol, 3.77 mL, 1.5 eq.) was added into the mixture at 0° C. and the mixture was stirred at 25° C. for 2.5 h. The reaction mixture was quenched by addition NH₄Cl (20 mL) at 0° C., and then extracted with DCM (20 mL×3). The combined organic layers were dried over Na₂SO₄, filtered and concentrated under reduced pressure to give a residue. The residue was purified by flash silica gel chromatography (DCM to DCM/MeOH=96/4) to yield 1-bromo-7-(4-methoxybenzyl)-6,7-dihydroimidazo[1,5-a]pyrazin-8(5H)-one (3 g, 8.30 mmol, 44.82% yield) as a white solid. LCMS [M+H]⁺=336.0, 338.0. ¹H NMR (400 MHz, DMSO-d₆) δ=7.82 (s, 1H), 7.25 (d, J=8.4 Hz, 2H), 6.91 (d, J=8.8 Hz, 2H), 4.56 (s, 2H), 4.26-4.20 (m, 2H), 3.73 (s, 3H), 3.60-3.55 (m, 2H).

Step E: A mixture of 1-bromo-7-(4-methoxybenzyl)-6,7-dihydroimidazo[1,5-a]pyrazin-8(5H)-one (800 mg, 2.38 mmol, 1 eq.), tert-BuBrettPhos-Pd-G3 (203.32 mg, 237.97 μmol, 0.1 eq.), Cs₂CO₃ (2.33 g, 7.14 mmol, 3 eq.) and MeOH (381.24 mg, 11.90 mmol, 481.49 μL, 5 eq.) in DMF (8 mL) was degassed and purged with N₂ for 3 times, and then the mixture was stirred at 90° C. for 2 h under N₂ atmosphere. The reaction mixture was diluted with EtOAc (10 mL×3), washed by water (5 mL) and brine (5 mL). The organic layer was dried over anhydrous sodium sulfate and concentrated under vacuum. The residue was purified by flash silica gel chromatography (DCM to DCM/MeOH=97/3) to yield 1-methoxy-7-(4-methoxybenzyl)-6,7-dihydroimidazo[1,5-a]pyrazin-8(5H)-one (450 mg, 1.25 mmol, 52.65% yield) as yellow oil. LCMS [M+H]⁺=288.1. ¹H NMR (400 MHz, DMSO-d₆) δ=7.47 (s, 1H), 7.21 (d, J=8.8 Hz, 2H), 6.89 (d, J=8.8 Hz, 2H), 4.52 (s, 2H), 4.15-4.10 (m, 2H), 3.85 (s, 3H), 3.73 (s, 3H), 3.48-3.44 (m, 2H).

Step F: To a solution 1-methoxy-7-(4-methoxybenzyl)-6,7-dihydroimidazo[1,5-a]pyrazin-8(5H)-one (110 mg, 382.86 μmol, 1 eq.) in THF (2 mL) was added LDA (2 M, 382.86 μL, 2 eq.) at −78° C. under N₂ atmosphere. The mixture was stirred at −78° C. for 0.5 h under N₂ atmosphere. Then methyl chloroformate (72.36 mg, 765.72 μmol, 59.16 μL, 2 eq.) was added into the mixture at −78° C. and the mixture was stirred at −78° C. for 1 h under N₂ atmosphere. Aqueous NH₄Cl (3 mL) was added into the mixture and the mixture was diluted with EtOAc (5 mL×3), washed by water (5 mL) and brine (5 mL). The organic layer was dried over anhydrous sodium sulfate and concentrated under vacuum. The residue was purified by flash silica gel chromatography (hexanes to hexanes/EtOAc=36/64) to yield methyl 1-methoxy-7-(4-methoxybenzyl)-8-oxo-5,6,7,8-tetrahydroimidazo[1,5-a]pyrazine-3-carboxylate (75 mg, 184.60 μmol, 48.22% yield) as a yellow solid. LCMS [M+H]⁺=346.1. ¹H NMR (400 MHz, DMSO-d₆) δ=7.23 (d, J=8.8 Hz, 2H), 6.90 (d, J=8.8 Hz, 2H), 4.58-4.47 (m, 4H), 3.89 (s, 3H), 3.83 (s, 3H), 3.73 (s, 3H), 3.56-3.51 (m, 2H).

Step G: To a solution of methyl 1-methoxy-7-(4-methoxybenzyl)-8-oxo-5,6,7,8-tetrahydroimidazo[1,5-a]pyrazine-3-carboxylate (115 mg, 333.00 μmol, 1 eq.) in DCM (2 mL) was added TfOH (499.75 mg, 3.33 mmol, 294.66 μL, 10 eq.) at 0° C. The mixture was stirred at 25° C. for 4 h. Then aqueous NaHCO₃ (350 mg, 10 eq.) was added into the mixture at 0° C. to quench the reaction, the reaction mixture was concentrated under vacuum to dryness. The crude product was purified by reversed-phase HPLC (Water/MeCN=5% to Water/MeCN=20%) to yield methyl 1-methoxy-8-oxo-5,6,7,8-tetrahydroimidazo[1,5-a]pyrazine-3-carboxylate (46 mg, 198.13 μmol, 59.50% yield) as a white solid. LCMS [M+H]⁺=226.1. ¹H NMR (400 MHz, DMSO-d₆) δ=7.85 (br s, 1H), 4.52-4.45 (m, 2H), 3.87 (s, 3H), 3.84 (s, 3H), 3.49-3.44 (m, 2H).

Step H: To a solution of methyl 1-methoxy-8-oxo-5,6,7,8-tetrahydroimidazo[1,5-a]pyrazine-3-carboxylate (36 mg, 159.86 μmol, 1 eq.) in THF (1 mL) was added NaBH₄ (30.24 mg, 799.29 μmol, 5 eq.) at 0° C. The mixture was stirred at 25° C. for 0.5 h. H₂O (2 mL) was added into the mixture to quench the reaction. The mixture was concentrated under reduced pressure to remove THF. The crude product was purified by reversed-phase HPLC (Water/MeCN=5%) to yield 3-(hydroxymethyl)-1-methoxy-6,7-dihydroimidazo[1,5-a]pyrazin-8(5H)-one (31 mg, 157.21 μmol, 98.34% yield) as a white solid. ¹H NMR (400 MHz, DMSO-d₆) δ=7.47 (br s, 1H), 5.47 (br s, 1H), 4.43 (s, 2H), 4.10-4.06 (m, 2H), 3.82 (s, 3H), 3.42-3.38 (m, 2H).

Step I: To a solution of 3-(hydroxymethyl)-1-methoxy-6,7-dihydroimidazo[1,5-a]pyrazin-8(5H)-one (20 mg, 101.42 μmol, 1 eq.) in DMF (0.5 mL) was added imidazole (13.81 mg, 202.85 μmol, 2 eq.) and TBSCl (27.52 mg, 182.56 μmol, 22.46 μL, 1.8 eq.). The mixture was stirred at 20° C. for 16 h. The reaction mixture was diluted with EtOAc (3 mL), washed by water (2 mL) and brine (2 mL). The organic layer was dried over anhydrous sodium sulfate and concentrated under vacuum. The residue was purified by flash silica gel chromatography (DCM to DCM/MeOH=96/4) to yield 3-(((tert-butyldimethylsilyl)oxy)methyl)-1-methoxy-6,7-dihydroimidazo[1,5-a]pyrazin-8(5H)-one (44 mg, 98.89 μmol, 97.50% yield) as a white solid. LCMS [M+H]⁺=312.2. ¹H NMR (400 MHz, DMSO-d₆) δ=7.50 (br s, 1H), 4.65 (s, 2H), 4.04 (br d, J=5.2 Hz, 2H), 3.82 (s, 3H), 3.45-3.39 (m, 2H), 0.85 (s, 9H), 0.05 (s, 6H).

### Intermediate 76: Synthesis of 3-bromo-1-methyl-1,5-dihydro-4H-pyrazolo[4,3-c]pyridin-4-one

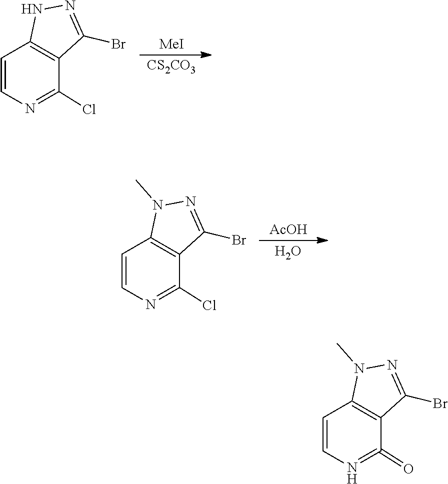

Step A: To a solution of 3-bromo-4-chloro-1H-pyrazolo[4,3-c]pyridine (3 g, 12.91 mmol, 1 eq.) in DMF (50 mL) was added MeI (5.50 g, 38.72 mmol, 2.41 mL, 3 eq.) and Cs₂CO₃ (8.41 g, 25.81 mmol, 2 eq.). The mixture was stirred at 20° C. for 4 h under N₂. The mixture was quenched with water (100 mL) and extracted with EtOAc (100 mL×2). The combined organic layer was washed with brine (80 mL), dried over Na₂SO₄, filtered and concentrated to give a residue, which was purified by flash silica gel chromatography (hexane to hexane/EtOAc=2/3, TLC: hexane/EtOAc=1/3, R_{f}=0.6) to give 3-bromo-4-chloro-1-methyl-1H-pyrazolo[4,3-c]pyridine (1.75 g, 7.10 mmol, 55.01% yield) as a white solid. ¹H NMR (400 MHz, CDCl₃) δ=8.19 (d, J=6.0 Hz, 1H), 7.24 (d, J=6.4 Hz, 1H), 4.05 (s, 3H).

Step B: To a solution of 3-bromo-4-chloro-1-methyl-1H-pyrazolo[4,3-c]pyridine (800 mg, 3.25 mmol, 1 eq.) in H₂O (5 mL) was added AcOH (20 mL) and the mixture was stirred at 100° C. for 12 h. Then, the reaction mixture was neutralized by sat. aq. NaHCO₃ and concentrated to give a residue, which was purified by flash silica gel chromatography (DCM to DCM/MeOH=90/10, TLC: DCM/MeOH=10/1, R_{f}=0.55) to give 3-bromo-1-methyl-1,5-dihydro-4H-pyrazolo[4,3-c]pyridin-4-one (721 mg, 2.85 mmol, 87.67% yield) as a white solid. ¹H NMR (400 MHz, DMSO-d₆) δ=11.12 (s, 1H), 7.23 (d, J=7.2 Hz, 1H), 6.58 (d, J=7.6 Hz, 1H), 3.89 (s, 3H).

### Intermediate 77: Synthesis of 2-bromo-4-((4,4-difluorochroman-6-yl)methyl)pyridine

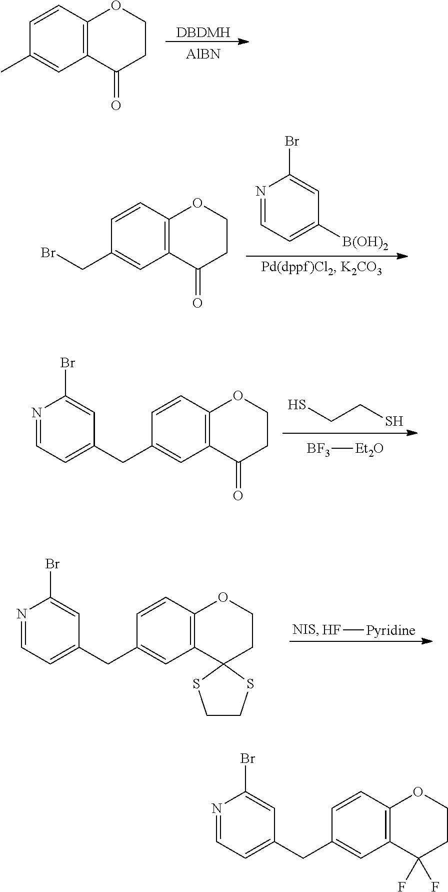

Step A: To a solution of 6-methylchroman-4-one (2 g, 12.33 mmol, 1 eq.) in PhCl (40 mL) was added AIBN (404.99 mg, 2.47 mmol, 0.2 eq.) and 1,3-dibromo-5,5-dimethylhydantoin (2.15 g, 7.52 mmol, 0.61 eq.). The mixture was stirred at 80° C. for 3 h. The mixture was extracted with DCM (100 mL×2). The combined organic layers were dried over Na₂SO₄, filtered and concentrated under reduced pressure to give a residue. The residue was purified by flash silica gel chromatography (hexane to hexane/EtOAc=20/80, TLC: hexane/EtOAc=3/1, R_{f}=0.65) to give 6-(bromomethyl) chroman-4-one (1.1 g, 4.56 mmol, 37.00% yield) as yellow oil. ¹H NMR (400 MHz, CDCl₃) δ=7.91 (d, J=2.0 Hz, 1H), 7.55-7.49 (m, 1H), 6.97 (d, J=8.6 Hz, 1H), 4.56-4.52 (m, 2H), 4.47 (s, 2H), 2.84-2.79 (m, 2H).

Step B: To a solution of 6-(bromomethyl) chroman-4-one (1.1 g, 4.56 mmol, 1 eq.) and (2-bromopyridin-4-yl) boronic acid (1.10 g, 5.48 mmol, 1.2 eq.) in dioxane (30 mL) and H₂O (3 mL) was added Pd(dppf) C12·CH₂Cl₂ (372.62 mg, 456.28 μmol, 0.1 eq.) and K₂CO₃ (1.26 g, 9.13 mmol, 2 eq.). The mixture was stirred at 85° C. for 3 h under N₂. The mixture was extracted with DCM (100 mL×2). The combined organic layers were dried over Na₂SO₄, filtered and concentrated under reduced pressure to give a residue. The residue was purified by flash silica gel chromatography (hexane to hexane/EtOAc=60/40, TLC: hexane/EtOAc=1/1, R_{f}=0.60) to give 6-((2-bromopyridin-4-yl)methyl) chroman-4-one (710 mg, 2.23 mmol, 48.91% yield) as a light-yellow solid. ¹H NMR (400 MHz, DMSO-d₆) δ=8.27 (d, J=4.8 Hz, 1H), 7.66 (d, J=2.4 Hz, 1H), 7.56 (s, 1H), 7.51-7.44 (m, 1H), 7.33-7.29 (m, 1H), 7.00 (d, J=8.8 Hz, 1H), 4.50 (t, J=6.4 Hz, 2H), 3.96 (s, 2H), 2.76 (t, J=6.8 Hz, 2H).

Step C: To a solution of 6-((2-bromopyridin-4-yl)methyl) chroman-4-one (600 mg, 1.89 mmol, 1 eq.) in DCM (25 mL) was added BF₃ Et₂O (722.65 mg, 5.09 mmol, 626.21 μL, 2.7 eq.) and ethane-1,2-dithiol (444.10 mg, 4.71 mmol, 395.46 μL, 2.5 eq.) at 0° C. The mixture was stirred at 20° C. for 14 h under N₂. The mixture was quenched with sat. aq. NaHCO₃ (80 mL) and extracted with DCM (100 mL×2). The combined organic layers were washed with brine (100 mL), dried over Na₂SO₄ and concentrated under reduced pressure to give a residue. The residue was purified by flash silica gel chromatography (hexane to hexane/EtOAc=80/20, TLC: hexane/EtOAc=3/1, R_{f}=0.48) to give 2-bromo-4-(spiro[chromane-4,2′-[1,3]dithiolan]-6-ylmethyl)pyridine (565 mg, 1.43 mmol, 75.97% yield) as yellow oil. LCMS [M+H]⁺=396.0.

Step D: To a solution of NIS (644.68 mg, 2.87 mmol, 2 eq.) in DCM (25 mL) was added dropwise HF-Pyridine (873.81 mg, 5.73 mmol, 794.37 μL, 65% purity, 4 eq.) at −70° C. under N₂. Then, a solution of 2-bromo-4-(spiro[chromane-4,2′-[1,3]dithiolan]-6-ylmethyl)pyridine (565 mg, 1.43 mmol, 1 eq.) in DCM (3 mL) was added dropwise at −70° C. under N₂. Then the mixture was stirred at −70° C. for 0.25 h under N₂. The mixture was added sat. aq. NaHCO₃ (100 mL) and extracted with DCM (100 mL×2). The combined organic layer was washed brine (80 mL), dried over Na₂SO₄, filtered and concentrated under reduced pressure to give a residue. The residue was purified by flash silica gel chromatography (hexane to hexane/EtOAc=50/50, TLC: hexane/EtOAc=2/1, R_{f}=0.64) to give 2-bromo-4-((4,4-difluorochroman-6-yl)methyl)pyridine (405 mg, 1.17 mmol, 81.44% yield) as a light-yellow solid. LCMS [M+H]⁺=340.0. ¹H NMR (400 MHz, CDCl₃) δ=8.26 (d, J=5.2 Hz, 1H), 7.40 (s, 1H), 7.29 (s, 1H), 7.11 (br d, J=8.4 Hz, 1H), 7.08-7.02 (m, 1H), 6.86 (d, J=8.4 Hz, 1H), 4.37-4.32 (m, 2H), 3.89 (s, 2H), 2.52-2.42 (m, 2H).

### Intermediate 78: Synthesis of 2-bromo-4-((2-(trifluoromethyl)pyridin-4-yl)methyl)pyridine

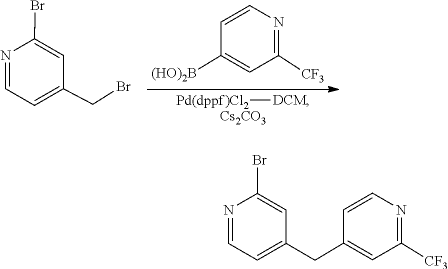

Step A: To a solution of (2-(trifluoromethyl)pyridin-4-yl) boronic acid (1 g, 5.24 mmol, 1 eq.) and 2-bromo-4-(bromomethyl)pyridine (1.58 g, 6.29 mmol, 1.2 eq.) in dioxane (40 mL) and H₂O (8 mL) were added Pd(dppf)Cl₂-DCM (427.75 mg, 523.79 μmol, 0.1 eq.) and K₂CO₃ (2.90 g, 20.95 mmol, 4 eq.) under N₂ atmosphere. The resulting mixture was stirred at 70° C. for 2 h under N₂ atmosphere. The reaction was cooled to room temperature, poured into water, and then extracted with EtOAc (100 mL×3). The organic phase was washed with brine (100 mL), dried over anhydrous Na₂SO₄, and filtered. The filtrate was concentrated under reduced pressure to give a residue, which was purified by silica gel chromatography (hexane to hexane/EtOAc=70/30) to give 2-bromo-4-((2-(trifluoromethyl)pyridin-4-yl)methyl)pyridine (890 mg, 2.71 mmol, 51.65% yield) as a brown solid. LCMS [M+H]⁺=317.0. ¹H NMR (400 MHz, DMSO-d₆) δ=8.69 (d, J=4.8 Hz, 1H), 8.32 (d, J=5.2 Hz, 1H), 7.90 (s, 1H), 7.68 (s, 1H), 7.63 (d, J=4.8 Hz, 1H), 7.39 (dd, J=1.2, 5.2 Hz, 1H), 4.15 (s, 2H).

### Intermediate 79: Synthesis of 4-((2-bromopyridin-4-yl)methyl)-2-methyl-6-(trifluoromethyl)pyridine

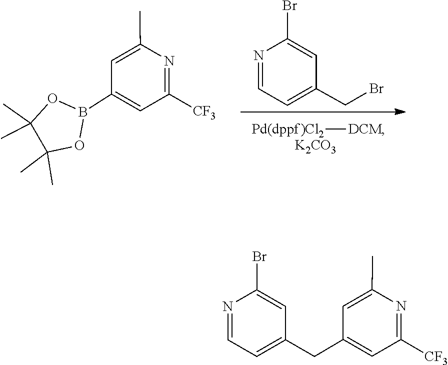

Step A: A mixture of 2-bromo-4-(bromomethyl)pyridine (1.05 g, 4.18 mmol, 1.2 eq.), 2-methyl-4-(4,4,5,5-tetramethyl-1,3,2-dioxaborolan-2-yl)-6-(trifluoromethyl)pyridine (1 g, 3.48 mmol, 1 eq.), Pd(dppf)Cl₂·CH₂Cl₂ (568.92 mg, 696.66 μmol, 0.2 eq.), K₂CO₃ (1.44 g, 10.45 mmol, 3 eq.) in dioxane (25 mL) and H₂O (2.5 mL) was degassed and purged with N₂ three times, and then the mixture was stirred at 50° C. for 1 h under N₂ atmosphere. The reaction mixture was concentrated under reduced pressure to give a residue. The residue was purified by flash silica gel chromatography (hexanes to hexanes/EtOAc=19/1, TLC: hexanes/EtOAc=5/1, R_{f}=0.4) to yield 4-((2-bromopyridin-4-yl)methyl)-2-methyl-6-(trifluoromethyl)pyridine (0.58 g, 1.75 mmol, 50.29% yield) as a white solid. ¹H NMR (400 MHz, CDCl₃) δ=8.33 (d, J=5.2 Hz, 1H), 7.31 (d, J=5.2 Hz, 2H), 7.12 (s, 1H), 7.06 (d, J=5.2 Hz, 1H), 3.98 (s, 2H), 2.62 (s, 3H).

### Intermediate 80: Synthesis of 2-bromo-4-(3-fluoro-4-(trifluoromethoxy)benzyl)pyridine

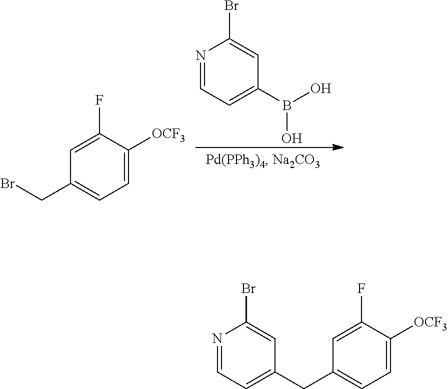 [SMILES: Fc1cc(CBr)ccc1OC(F)(F)F ; OB(O)c1ccnc(Br)c1 ; Fc1cc(Cc2ccnc(Br)c2)ccc1OC(F)(F)F]

Step A: To a solution of 4-(bromomethyl)-2-fluoro-1-(trifluoromethoxy)benzene (0.5 g, 1.83 mmol, 1 eq.) in toluene (10 mL) and EtOH (5 mL) was added (2-bromopyridin-4-yl) boronic acid (406.55 mg, 2.01 mmol, 1.1 eq.), Pd(PPh₃)₄ (211.62 mg, 183.14 μmol, 0.1 eq.) and Na₂CO₃ (776.42 mg, 7.33 mmol, 4 eq.). The mixture was degassed and purged with N₂ three times, then the mixture was stirred at 85° C. for 2 h under N₂ atmosphere. The reaction mixture was cooled to 20° C., filtered and concentrated under vacuum. The crude was purified by flash silica gel chromatography (hexanes to hexanes/EtOAc=85/15) to yield 2-bromo-4-(3-fluoro-4-(trifluoromethoxy)benzyl)pyridine (0.36 g, 1.03 mmol, 56.15% yield) as yellow oil. ¹H NMR 400 MHz, CDCl₃) δ=8.32 (d, J=5.2 Hz, 1H), 7.34-7.31 (m, 1H), 7.30-7.28 (m, 1H), 7.10-6.95 (m, 3H), 3.96 (s, 2H).

### Intermediate 81: Synthesis of 1-(2-(benzyloxy)ethyl)-1,5-dihydro-4H-[1,2,3]triazolo[4,5-c]pyridin-4-one

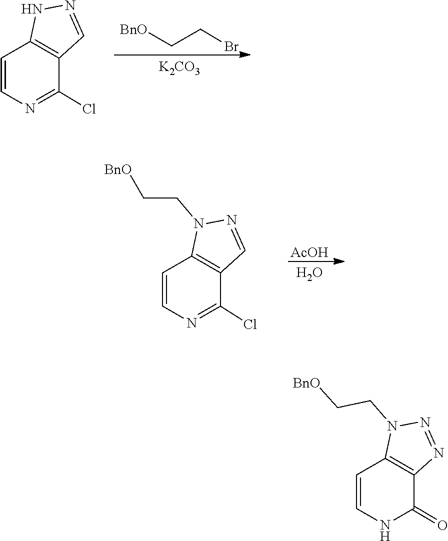

Step A: To a solution of 4-chloro-1H-[1,2,3]triazolo[4,5-c]pyridine (1 g, 6.47 mmol, 1 eq.) and ((2-bromoethoxy)methyl) benzene (1.81 g, 8.41 mmol, 1.33 mL, 1.3 eq.) in DMF (15 mL) was added K₂CO₃ (2.68 g, 19.41 mmol, 3 eq.), the mixture was stirred at 60° C. for 12 h under N₂ atmosphere. The reaction mixture was filtrated and concentrated under vacuum. The residue was purified by flash silica gel chromatography (hexane to hexane/EtOAc=70/30, TLC: hexane/EtOAc=1/1, R_{f}=0.6) to yield 1-(2-(benzyloxy)ethyl)-4-chloro-1H-[1,2,3]triazolo[4,5-c]pyridine (650 mg, 2.25 mmol, 34.79% yield) as brown oil. ¹H NMR (400 MHz, DMSO-d₆) δ=8.34 (d, J=6.0 Hz, 1H), 7.96 (d, J=6.0 Hz, 1H), 7.27-7.14 (m, 3H), 7.12-6.96 (m, 2H), 5.01 (t, J=5.2 Hz, 2H), 4.43 (s, 2H), 3.92 (t, J=4.8 Hz, 2H).

Step B: A mixture of 1-(2-(benzyloxy)ethyl)-4-chloro-1H-[1,2,3]triazolo[4,5-c]pyridine (600 mg, 2.08 mmol, 1 eq.) in AcOH (5 mL) and H₂O (5 mL) was stirred at 100° C. for 12 h under N₂ atmosphere. The reaction mixture was filtrated and concentrated under vacuum to yield 1-(2-(benzyloxy)ethyl)-1,5-dihydro-4H-[1,2,3]triazolo[4,5-c]pyridin-4-one (420 mg, crude) as a white solid. ¹H NMR (400 MHz, DMSO-d₆) δ=11.50 (br s, 1H), 7.37-7.22 (m, 4H), 7.16-7.06 (m, 2H), 6.71 (d, J=7.2 Hz, 1H), 4.79 (t, J=5.2 Hz, 2H), 4.44 (s, 2H), 3.86 (t, J=4.8 Hz, 2H).

### Intermediate 82: Synthesis of 2-bromo-4-((3-methyl-4-(trifluoromethyl)-1H-pyrazol-1-yl)methyl)pyridine

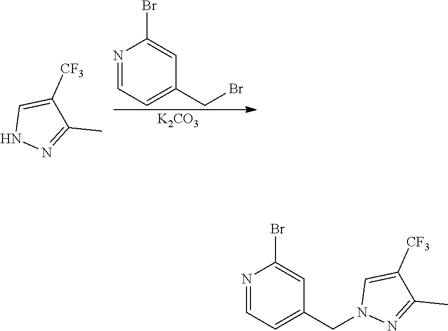

Step A: To a solution of 3-methyl-4-(trifluoromethyl)-1H-pyrazole (500 mg, 3.33 mmol, 1 eq.) in DMF (10 mL) was added K₂CO₃ (1.38 g, 9.99 mmol, 3 eq.) and 2-bromo-4-(bromomethyl)pyridine (1.00 g, 4.00 mmol, 1.2 eq.). The reaction mixture was stirred at 20° C. for 12 h. The reaction mixture was poured into water (20 mL) and extracted with EtOAc (30 mL×3). The combined organic layers were washed with water (30 mL×3), brine (30 mL×3) and dried over Na₂SO₄, filtered and concentrated under reduced pressure. The residue was purified by flash silica gel chromatography (hexanes to hexanes/EtOAc=70/30, TLC: hexanes/EtOAc=3/1) to give mixture of 2-bromo-4-((3-methyl-4-(trifluoromethyl)-1H-pyrazol-1-yl)methyl)pyridine and an isomer (880 mg, 2.75 mmol, 82.53% yield) as colorless oil. ¹H NMR (400 MHz, DMSO-d₆) δ=8.44 (s, 1H), 8.38-8.34 (m, 1H), 7.88 (s, 1H), 7.50 (s, 1H), 7.41 (s, 1H), 7.21 (dd, J=1.2, 5.2 Hz, 1H), 7.11-7.06 (m, 1H), 5.48 (s, 1H), 5.37 (s, 2H), 2.36 (s, 1H), 2.24 (s, 3H).

### Intermediate 83: Synthesis of 1-(2-(benzyloxy)ethyl)-3-methyl-3,5-dihydro-1H-imidazo[4,5-c]pyridine-2,4-dione

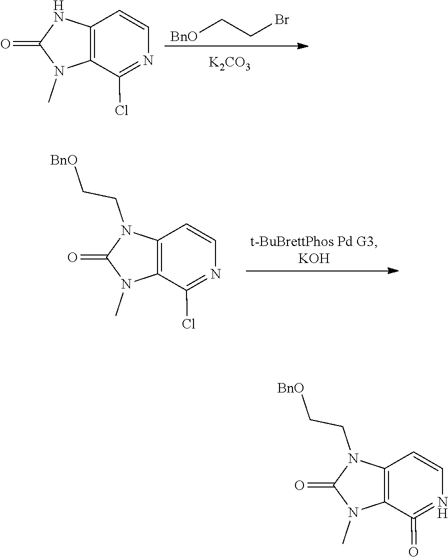

Step A: To a solution of 4-chloro-3-methyl-1,3-dihydro-2H-imidazo[4,5-c]pyridin-2-one (600 mg, 3.27 mmol, 1 eq.), ((2-bromoethoxy)methyl) benzene (4.22 g, 19.61 mmol, 3.10 mL, 6 eq.) in DMF (10 mL) was added K₂CO₃ (1.35 g, 9.80 mmol, 3 eq.). The mixture was stirred at 60° C. for 3 h. The mixture was cooled and quenched with H₂O (40 mL), then extracted with EtOAc (60 mL×2). The combined organics were washed with brine (30 mL), dried over Na₂SO₄, concentrated under vacuum to a residue, which was purified with silica gel chromatography (hexanes to hexane/EtOAc=1/1, TLC: hexane/EtOAc=1/1, R_{f}=0.15) to give 1-(2-(benzyloxy)ethyl)-4-chloro-3-methyl-1,3-dihydro-2H-imidazo[4,5-c]pyridin-2-one (750 mg, 2.36 mmol, 72.22% yield) as a white solid. ¹H NMR (400 MHz, CDCl₃) δ=8.05 (d, J=5.2 Hz, 1H), 7.32-7.29 (m, 2H), 7.19-7.14 (m, 2H), 7.07 (d, J=5.2 Hz, 1H), 4.48 (s, 2H), 4.10 (t, J=5.2 Hz, 2H), 3.78 (t, J=5.2 Hz, 2H), 3.74 (s, 3H)

Step B: To a solution of 1-(2-(benzyloxy)ethyl)-4-chloro-3-methyl-1,3-dihydro-2H-imidazo[4,5-c]pyridin-2-one (750 mg, 2.36 mmol, 1 eq.), KOH (1.32 g, 23.60 mmol, 10 eq.) in dioxane (12 mL) and H₂O (4 mL) was added tert-BuBrettPhos-Pd-G3 (403.32 mg, 472.04 μmol, 0.2 eq.). The mixture was stirred at 100° C. for 4 h under N₂. The mixture was cooled and concentrated under vacuum to a residue, which was purified with flash silica gel chromatography (DCM to DCM/MeOH=95/5, TLC: DCM/MeOH=95/5, R_{f}=0.09) to give 1-(2-(benzyloxy)ethyl)-3-methyl-3,5-dihydro-1H-imidazo[4,5-c]pyridine-2,4-dione (550 mg, 1.84 mmol, 77.85% yield) as a white solid. ¹H NMR (400 MHz, DMSO-d₆) δ=11.46 (br s, 1H), 7.35-7.09 (m, 6H), 6.46 (d, J=6.8 Hz, 1H), 4.46 (s, 2H), 3.98 (t, J=5.2 Hz, 2H), 3.64 (t, J=5.2 Hz, 2H), 3.51 (s, 3H)

### Intermediate 84: Synthesis of(S)-6-((benzyloxy)methyl)-1-methyl-6,7-dihydroimidazo[1,5-a]pyrazin-8(5H)-one

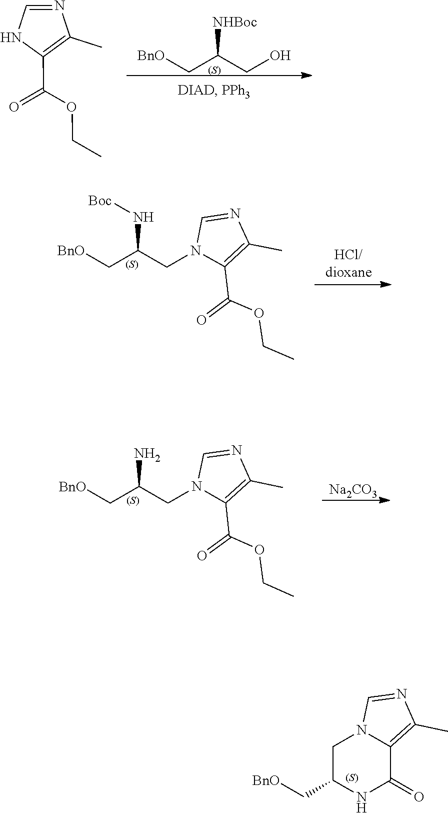

Step A: To a solution of ethyl 4-methyl-1H-imidazole-5-carboxylate (986.32 mg, 6.40 mmol, 1.2 eq.) in THF (15 mL) was added tert-butyl(S)-(1-(benzyloxy)-3-hydroxypropan-2-yl)carbamate (1.5 g, 5.33 mmol, 1 eq.), PPh₃ (1.82 g, 6.93 mmol, 1.3 eq.) and DIAD (1.40 g, 6.93 mmol, 1.34 mL, 1.3 eq.) at 0° C. After addition, the reaction mixture was heated to 50° C. and stirred for 16 h under N₂ atmosphere. The reaction was slowly quenched with H₂O (50 mL). The layers were separated, and the aqueous layer was extracted with EtOAc (50 mL×3). The combined organic layers were washed with brine (50 mL), dried over Na₂SO₄, filtered and concentrated to yield a residue. The residue was purified by flash silica gel chromatography (hexanes to hexanes/EtOAc=78/22, TLC: hexanes/EtOAc=1/1) to give ethyl(S)-1-(3-(benzyloxy)-2-((tert-butoxycarbonyl)amino)propyl)-4-methyl-1H-imidazole-5-carboxylate (2.6 g, 1.25 mmol, 23.36%) as a white solid. LCMS [M+H]⁺=418.2.

Step B: A solution of ethyl(S)-1-(3-(benzyloxy)-2-((tert-butoxycarbonyl)amino)propyl)-4-methyl-1H-imidazole-5-carboxylate (1.0 g, 479.04 μmol, 1 eq.) in HCl/dioxane (2 M, 10 mL) was stirred at 20° C. for 1 h. The mixture was concentrated under reduced pressure, add 50 ml of saturated brine to the residue, washed with EtOAc (30 mL×3). The aqueous phase was adjusted the pH=9 with sat. NaHCO₃ solution, then extracted with DCM (30 mL×3). The organic phase was washed with sat. brine (50 mL), dried over Na₂SO₄, filtered, concentrated to give ethyl(S)-1-(2-amino-3-(benzyloxy)propyl)-4-methyl-1H-imidazole-5-carboxylate (150 mg, crude) as yellow oil, which was used for the next step without further purification. LCMS [M+H]⁺=318.2. ¹H NMR (400 MHz, CDCl₃) δ=7.44 (s, 1H), 7.40-7.28 (m, 5H), 4.57-4.48 (m, 2H), 4.40 (dd, J=5.6, 13.2 Hz, 1H), 4.31 (q, J=7.2 Hz, 2H), 4.13 (dd, J=7.2, 13.6 Hz, 1H), 3.55-3.44 (m, 1H), 3.43-3.28 (m, 2H), 2.48 (s, 3H), 1.37 (t, J=7.2 Hz, 3H).

Step C: A solution of ethyl(S)-1-(2-amino-3-(benzyloxy)propyl)-4-methyl-1H-imidazole-5-carboxylate (990 mg, 3.12 mmol, 1 eq.) and Na₂CO₃ (991.83 mg, 9.36 mmol, 3 eq.) in MeOH (15 mL) was stirred at 50° C. for 16 h. The reaction was slowly quenched with H₂O (30 mL). The layers were separated and the aqueous layer was extracted with EtOAc (20 mL×3). The combined organic layers were washed with brine (30 mL×2), dried over Na2SO₄, filtered and concentrated to yield a residue. The residue was purified by flash silica gel chromatography (DCM to DCM/MeOH=96/4, TLC: DCM/MeOH=10/1) to give(S)-6-((benzyloxy)methyl)-1-methyl-6,7-dihydroimidazo[1,5-a]pyrazin-8(5H)-one (810 mg, 2.84 mmol, 90.93% yield) as yellow oil. LCMS [M+H]⁺=272.1. ¹H NMR (400 MHz, CDCl₃) δ=7.45 (s, 1H), 7.41-7.27 (m, 5H), 6.00 (s, 1H), 4.61-4.47 (m, 2H), 4.22-4.15 (m, 1H), 4.11-3.97 (m, 2H), 3.52 (d, J=6.4 Hz, 2H), 2.54 (s, 3H).

### Intermediate 85: Synthesis of 1,3-dimethyl-1,4,5,6-tetrahydro-7H-pyrazolo[3,4-c]pyridin-7-one

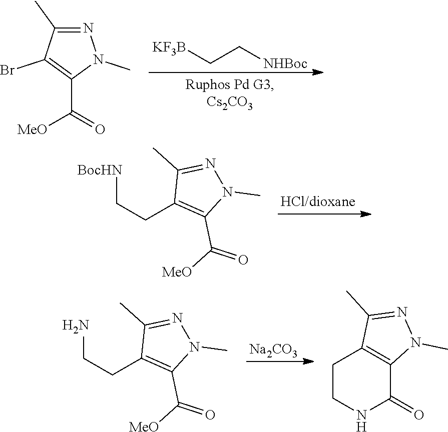

Step A: A mixture of methyl 4-bromo-1,3-dimethyl-1H-pyrazole-5-carboxylate (500 mg, 2.15 mmol, 1 eq.), potassium tert-butyl N-(2-(trifluoroboranuidyl)ethylcarbamate (808.03 mg, 3.22 mmol, 1.5 eq.), RuPhos Pd G3 (179.43 mg, 214.54 μmol, 0.1 eq.), and Cs₂CO₃ (2.10 g, 6.44 mmol, 3 eq.) in toluene (6 mL) and H₂O (2 mL) was degassed and purged with N₂ three times, then the mixture was stirred at 80° C. for 16 h under N₂ atmosphere. The reaction mixture was cooled and diluted with EtOAc (10 mL×3), washed by water (5 mL) and brine (5 mL). The organic layer was dried over Na₂SO₄ and concentrated in vacuum to give the residue, which was purified by flash silica gel chromatography (hexane to hexane/EtOAc=80/20, TLC: hexane/EtOAc=3/1, R_{f}=0.4) to give methyl 4-(2-((tert-butoxycarbonyl)amino)ethyl)-1,3-dimethyl-1H-pyrazole-5-carboxylate (520 mg, 1.57 mmol, 73.36% yield) as yellow oil. LCMS [M+H]⁺=298.1.

Step B: A mixture of methyl 4-(2-((tert-butoxycarbonyl)amino)ethyl)-1,3-dimethyl-1H-pyrazole-5-carboxylate (200 mg, 642.31 μmol, 1 eq.) in MeOH (2 mL) was added HCl/dioxane (2 M, 3.63 mL, 11.31 eq.) at 25° C. The mixture was stirred at 25° C. for 4 h. The reaction mixture was concentrated under reduced pressure to give methyl 4-(2-aminoethyl)-1,3-dimethyl-1H-pyrazole-5-carboxylate (105 mg, crude, HCl salt) as a white solid, which was used directly in the next step without further purification. LCMS [M+H]⁺=198.1.

Step C: To a solution of methyl 4-(2-aminoethyl)-1,3-dimethyl-1H-pyrazole-5-carboxylate (96.40 mg, 456.31 μmol, 1 eq.) in MeOH (4 mL) was added Na₂CO₃ (193.46 mg, 1.83 mmol, 4 eq.). The mixture was stirred at 25° C. for 4 h. The mixture was poured into water (10 mL) and the aqueous phase was extracted with EtOAc (20 mL×3). The combined organic phase was washed with brine (50 mL), dried over anhydrous Na₂SO₄, filtered and concentrated under vacuum to give 1,3-dimethyl-1,4,5,6-tetrahydro-7H-pyrazolo[3,4-c]pyridin-7-one (70 mg, crude) as a white solid, which was used directly in the next step without further purification. LCMS [M+H]⁺=166.1. ¹H NMR (400 MHz, DMSO-d₆) δ=7.68 (s, 1H) 3.95 (s, 3H) 3.37-3.32 (m, 2H) 2.58 (t, J=6.8 Hz, 2H) 2.11 (s, 3H).

### Intermediate 86: Synthesis of 3-(2-(benzyloxy) ethoxy)-1-methyl-1,4,5,6-tetrahydro-7H-pyrazolo[3,4-c]pyridin-7-one

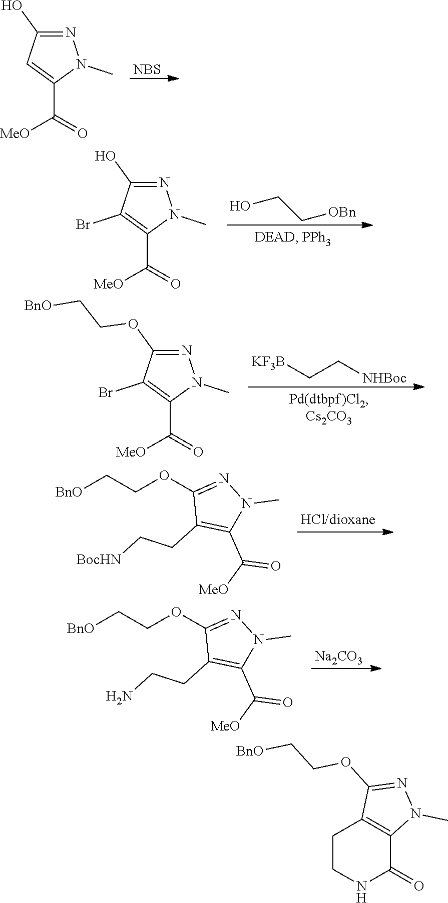

Step A: To a solution of methyl 3-hydroxy-1-methyl-1H-pyrazole-5-carboxylate (500 mg, 3.20 mmol, 1 eq.) in DMF (5 mL) was added NBS (854.93 mg, 4.80 mmol, 1.5 eq.) at 0° C. The mixture was stirred at 25° C. for 2 h. The reaction mixture was diluted with EtOAc (50 mL×3), washed by water (30 mL) and brine (30 mL). The organic layer was dried over anhydrous sodium sulfate and concentrated under vacuum. The residue was purified by flash silica gel chromatography (hexanes to hexanes/EtOAc=70/30) to yield methyl 4-bromo-3-hydroxy-1-methyl-1H-pyrazole-5-carboxylate (430 mg, 1.65 mmol, 51.42% yield) as a yellow solid. LCMS [M+H]⁺=235.0. ¹H NMR (400 MHz, DMSO-d₆) δ=10.75 (s, 1H), 3.89 (s, 3H), 3.84 (s, 3H).

Step B: To a solution of methyl 4-bromo-3-hydroxy-1-methyl-1H-pyrazole-5-carboxylate (330 mg, 1.40 mmol, 1 eq.) and 2-(benzyloxy) ethan-1-ol (427.36 mg, 2.81 mmol, 399.03 μL, 2 eq.) in THF (3 mL) was added PPh₃ (736.52 mg, 2.81 mmol, 2 eq.) and DEAD (489.04 mg, 2.81 mmol, 510.48 μL, 2 eq.) at 0° C. under N₂ atmosphere. The mixture was stirred at 25° C. for 1 h under N₂ atmosphere. The reaction mixture was diluted with EtOAc (20 mL×3), washed by water (15 mL) and brine (15 mL). The organic layer was dried over anhydrous sodium sulfate and concentrated under vacuum. The residue was purified by flash silica gel chromatography (hexanes to hexanes/EtOAc=97/3) to yield methyl 3-(2-(benzyloxy) ethoxy)-4-bromo-1-methyl-1H-pyrazole-5-carboxylate (420 mg, 1.08 mmol, 76.97% yield) as yellow oil. LCMS [M+H]⁺=369.1. ¹H NMR (400 MHz, DMSO-d₆) δ=7.16-7.44 (m, 5H), 4.55 (s, 2H), 4.30-4.37 (m, 2H), 3.94 (s, 3H), 3.87 (s, 3H), 3.73-3.78 (m, 2H).

Step C: A mixture of methyl 3-(2-(benzyloxy) ethoxy)-4-bromo-1-methyl-1H-pyrazole-5-carboxylate (150 mg, 406.27 μmol, 1 eq.), potassium tert-butyl N-(2-(trifluoroboranuidyl)ethyl)carbamate (510.07 mg, 2.03 mmol, 5 eq.), Cs₂CO₃ (397.11 mg, 1.22 mmol, 3 eq.), and Pd(dtbpf)Cl₂ (26.48 mg, 40.63 μmol, 0.1 eq.) in toluene (1.6 mL) and H₂O (0.4 mL) was degassed and purged with N₂ three times, then the mixture was stirred at 90° C. for 2 h under N₂ atmosphere. The reaction mixture was cooled and diluted with EtOAc (5 mL×3), washed by water (3 mL) and brine (3 mL). The organic layer was dried over anhydrous sodium sulfate and concentrated under vacuum. The residue was purified by flash silica gel chromatography (hexanes to hexanes/EtOAc=80/20) to yield methyl 3-(2-(benzyloxy) ethoxy)-4-(2-((tert-butoxycarbonyl)amino)ethyl)-1-methyl-1H-pyrazole-5-carboxylate (135 mg, 217.99 μmol, 53.66% yield) as yellow oil. LCMS [M+H]⁺=434.1. ¹H NMR (400 MHz, DMSO-d₆) δ=7.26-7.44 (m, 4H), 6.78 (br t, J=5.6 Hz, 1H), 4.55 (s, 2H), 4.24-4.31 (m, 2H), 3.88 (s, 3H), 3.83 (s, 3H), 3.70-3.75 (m, 2H), 2.97-3.10 (m, 2H), 2.57-2.69 (m, 2H), 1.35 (s, 9H).

Step D: A mixture of methyl 3-(2-(benzyloxy) ethoxy)-4-(2-((tert-butoxycarbonyl)amino)ethyl)-1-methyl-1H-pyrazole-5-carboxylate (100 mg, 230.68 μmol, 1 eq.) in HCl/dioxane (1.5 mL) was stirred at 25° C. for 0.5 h. The mixture was concentrated under reduced pressure to give methyl 4-(2-aminoethyl)-3-(2-(benzyloxy) ethoxy)-1-methyl-1H-pyrazole-5-carboxylate (90 mg, crude, HCl) as a white solid, which was used in the next step without further purification. LCMS [M+H]⁺=334.1.

Step E: To a solution of methyl 4-(2-aminoethyl)-3-(2-(benzyloxy) ethoxy)-1-methyl-1H-pyrazole-5-carboxylate (90 mg, 243.35 μmol, 1 eq, HCl salt) in MeOH (2 mL) was added Na₂CO₃ (128.96 mg, 1.22 mmol, 5 eq.). The mixture was stirred at 80° C. for 3 h. The reaction mixture was diluted with EtOAc (5 mL×3), washed by water (3 mL) and brine (3 mL). The organic layer was dried over anhydrous sodium sulfate and concentrated under vacuum. The residue was purified by flash silica gel chromatography (hexanes to hexanes/EtOAc=42/58) to yield 3-(2-(benzyloxy) ethoxy)-1-methyl-1,4,5,6-tetrahydro-7H-pyrazolo[3,4-c]pyridin-7-one (60 mg, 179.20 μmol, 73.64% yield) as a white solid. LCMS [M+H]⁺=302.2. ¹H NMR (400 MHz, DMSO-d₆) δ=7.77 (br s, 1H), 7.16-7.42 (m, 5H), 4.53 (s, 2H), 4.24-4.32 (m, 2H), 3.89 (s, 3H), 3.69-3.75 (m, 2H), 3.37 (br d, J=3.2 Hz, 2 H), 3.31 (br s, 1H).

### Intermediate 87: Synthesis of 1-bromo-3-(2-hydroxyethyl)-6,7-dihydroimidazo[1,5-a]pyrazin-8(5H)-one

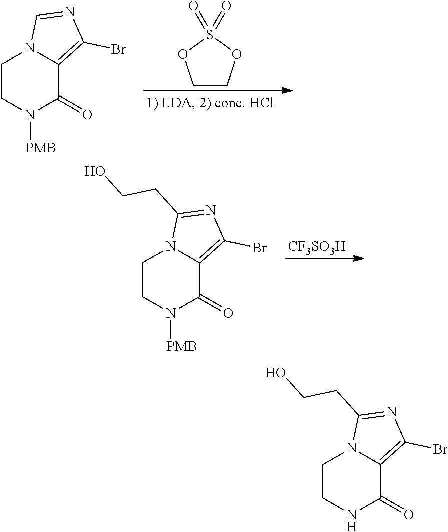

Step A: First, three solutions were prepared. Solution 1:1-bromo-7-(4-methoxybenzyl)-6,7-dihydroimidazo[1,5-a]pyrazin-8(5H)-one (1 g, 2.97 mmol, 1 eq.) in THE, 40 mL. Solution 2: LDA (2 M, 2.68 mL, 1.8 eq.). Solution 3:1,2-ethylene sulfate (922.98 mg, 7.44 mmol, 2.5 eq.) in THF, 25 mL. Solution 1 was pumped {19.111 mL/min} to flow reactor 1 {PFA, Coils reactor, 1.590 ( 1/16″) mm, 0.540 mL, −30.0° C.}. Solution 2 was pumped {2.489 mL/min} to flow reactor 1. Solution 3 was pumped {12.1 mL/min} to flow reactor 2 {PFA, Coils reactor, 1.590 ( 1/16″) mm, 0.540 mL, −30.0° C.}. The residence time of flow reactor 1 was 0.025 min. The residence time of flow reactor 2 was 0.016 min. The output of flow reactor 2 was collected into a bottle and quenched by 8.0 mL aq. HCl at 0.0° C.). The reaction mixture was diluted with aq. NaHCO₃30 mL at 0° C., then extracted with EtOAc (40 mL×3). The combined organic layers were dried over Na₂SO₄, filtered and concentrated under reduced pressure to give a residue. The residue was purified by flash silica gel chromatography (DCM to DCM/MeOH=96/4) to yield 1-bromo-3-(2-hydroxyethyl)-7-(4-methoxybenzyl)-6,7-dihydroimidazo[1,5-a]pyrazin-8(5H)-one (390 mg, 871.83 μmol, 29.31% yield) as a yellow solid. LCMS [M+H]⁺=380.1. ¹H NMR (400 MHz, DMSO-d₆) δ=7.30-7.16 (m, 2H), 6.95-6.88 (m, 2H), 4.87-4.76 (m, 1H), 4.63-4.40 (m, 2H), 4.15-4.09 (m, 2H), 3.73 (s, 3H), 3.69-3.63 (m, 2H), 3.60-3.45 (m, 2H), 2.84-2.62 (m, 2H).

Step B: To a solution of 1-bromo-3-(2-hydroxyethyl)-7-(4-methoxybenzyl)-6,7-dihydroimidazo[1,5-a]pyrazin-8(5H)-one (390 mg, 1.03 mmol, 1 eq.) in toluene (5 mL) was added TfOH (1.54 g, 10.26 mmol, 907.61 μL, 10 eq.) at 0° C. The mixture was stirred at 25° C. for 3 h. Then, aq. NaHCO₃ was added into the mixture to bring the solution to pH=8. The mixture was diluted with EtOAc (2 mL×2), washed by water (4 mL×3). The aqueous phase was collected for purification. The crude product was purified by reversed-phase HPLC (ACN/H₂O)(Water/MeCN=1% to 10%) to yield 1-bromo-3-(2-hydroxyethyl)-6,7-dihydroimidazo[1,5-a]pyrazin-8(5H)-one (150 mg, 553.66 μmol, 53.98% yield) as a white solid. LCMS [M+H]⁺=260.0. ¹H NMR (400 MHz, DMSO-d₆) δ=8.05-7.88 (m, 1H), 4.89-4.56 (m, 1H), 4.17-4.06 (m, 2H), 3.67 (t, J=6.4 Hz, 2H), 3.49-3.44 (m, 2H), 2.80 (t, J=6.4 Hz, 2H).

### Intermediate 88: Synthesis of 3-(2-hydroxyethoxy)-1-methyl-6,7-dihydroimidazo[1,5-a]pyrazin-8(5H)-one

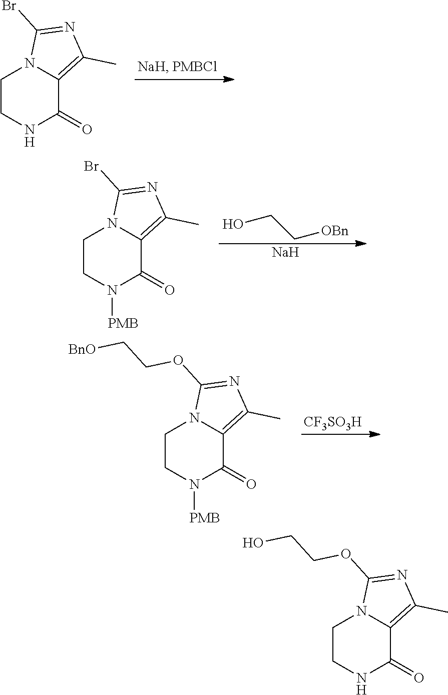

Step A: To a solution of 3-bromo-1-methyl-6,7-dihydroimidazo[1,5-a]pyrazin-8(5H)-one (3 g, 13.04 mmol, 1 eq.) in DMF (30 mL) was added NaH (782.32 mg, 19.56 mmol, 60% purity, 1.5 eq.) at 0° C. under N₂ and the mixture was stirred at 0° C. for 0.5 h. Then PMB-C1 (3.06 g, 19.56 mmol, 2.65 mL, 1.5 eq.) was added at 0° C. and the reaction was stirred at 25° C. for 2.5 h under N₂ atmosphere. Then, the reaction mixture was quenched by addition H₂O (10 mL) at 0° C., and the mixture was concentrated under reduced pressure to give a residue. The residue was purified by flash silica gel chromatography (hexanes to hexanes/EtOAc=1/1) to give 3-bromo-7-(4-methoxybenzyl)-1-methyl-6,7-dihydroimidazo[1,5-a]pyrazin-8(5H)-one (4 g, 11.42 mmol, 87.59% yield) as yellow oil. ¹H NMR (400 MHz, DMSO-d₆) δ=7.24 (d, J=8.4 Hz, 2H), 6.90 (d, J=8.8 Hz, 2H), 4.56 (s, 2H), 4.07-4.01 (m, 2H), 3.73 (s, 3H), 3.59-3.53 (m, 2H), 2.37 (s, 3H).

Step B: To a suspension of NaH (282.66 mg, 7.07 mmol, 60% purity, 5 eq.) in DMF (10 mL) was added 2-(benzyloxy) ethan-1-ol (430.22 mg, 2.83 mmol, 401.70 μL, 2 eq.) at 25° C. under N₂, the mixture was stirred at 25° C. for 30 min under N₂. Then, 3-bromo-7-(4-methoxybenzyl)-1-methyl-6,7-dihydroimidazo[1,5-a]pyrazin-8(5H)-one (550 mg, 1.41 mmol, 1 eq.) was added at 25° C. The reaction mixture was stirred at 60° C. for 1.5 h under N₂. The reaction mixture was quenched with sat. NH₄Cl (20 mL) and extracted with EtOAc (20 mL×3). The combined organic layers were washed with water (20 mL×3), brine (20 mL×3) and dried over Na₂SO₄, filtered and concentrated under reduced pressure. The residue was purified by flash silica gel chromatography (hexanes to hexanes/EtOAc (0.1% of NH₃·H₂O)=25/75) to give 3-(2-(benzyloxy) ethoxy)-7-(4-methoxybenzyl)-1-methyl-6,7-dihydroimidazo[1,5-a]pyrazin-8(5H)-one (550 mg, 1.30 mmol, 92.32% yield) as yellow gum. ¹H NMR (400 MHz, DMSO-d₆) δ=7.34-7.25 (m, 5H), 7.22 (d, J=8.5 Hz, 2H), 6.92-6.87 (m, 2H), 4.55-4.51 (m, 4H), 4.49-4.42 (m, 2H), 3.86-3.79 (m, 2H), 3.75-3.73 (m, 2H), 3.73 (s, 3H), 3.47-3.41 (m, 2H), 2.31 (s, 3H).

Step C: To a solution of 3-(2-(benzyloxy) ethoxy)-7-(4-methoxybenzyl)-1-methyl-6,7-dihydroimidazo[1,5-a]pyrazin-8(5H)-one (500 mg, 1.19 mmol, 1 eq.) in toluene (10 mL) was added CF₃SO₃H (1.78 g, 11.86 mmol, 1.05 mL, 10 eq.) at 0° C. The reaction mixture was stirred at 25° C. for 2 h. The reaction mixture was adjusted to pH=7 with sat. aq. NaHCO₃ and washed with EtOAc (10 mL×3). The aqueous phase was concentrated under reduced pressure to give residue. The residue was purified by reversed-phase HPLC (0.1% NH₃·H₂O condition) to give 3-(2-hydroxyethoxy)-1-methyl-6,7-dihydroimidazo[1,5-a]pyrazin-8(5H)-one (240 mg, 1.14 mmol, 95.78% yield) as a white solid. ¹H NMR (400 MHz, DMSO-d₆) δ=7.53 (br. s, 1H), 4.93 (br. s, 1H), 4.35-4.23 (m, 2H), 3.83-3.76 (m, 2H), 3.68 (t, J=4.8 Hz, 2H), 3.43-3.36 (m, 2H), 2.26 (s, 3H).

### Intermediate 89: Synthesis of 4-chloro-6-(3-(difluoromethyl)-5-fluorobenzyl)pyrimidine

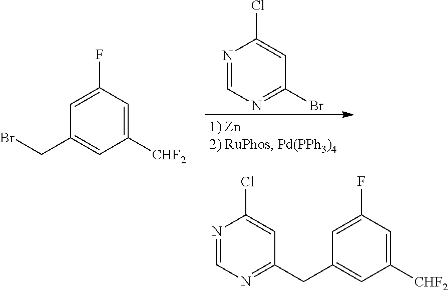 [SMILES: Fc1cc(CBr)cc(C(F)F)c1 ; Clc1cc(Br)ncn1 ; Fc1cc(Cc2cc(Cl)ncn2)cc(C(F)F)c1]

Step A: A pre-prepared Zn powder column was flushed at a flow rate of 1 mL/min with anhydrous THF (10 mL). A solution of TMSCl (0.8 mL) and 1,2-dibromoethane (0.2 mL) in THF (10 mL) was run through the column to activate the zinc powder at 50° C. and a flow rate 1 mL/min with plunger pump. The column was flushed once more at flow rate of 1.0 mL/min with anhydrous THF (10 mL). Then a solution of 1-(bromomethyl)-3-(difluoromethyl)-5-fluorobenzene (1 g, 4.18 mmol, 1 eq.) in anhydrous THF (9.5 mL) was passed through the column at 50° C. and a flow rate 0.5 mL/min. A yellow solution (solution 1, 0.27M) was collected from the output and titrated with a solution of iodine. A second solution of 4-bromo-6-chloropyrimidine (220 mg, 1.14 mmol, 1 eq.), RuPhos (18.58 mg, 39.81 μmol, 0.035 eq.) and Pd(PPh₃)₄ (32.86 mg, 28.43 μmol, 0.025 eq.) in THF (3 mL) was prepared. The organozinc solution 1 (0.27 M, 4.21 mL, 1 eq.) was pumped {1 mL/min} to flow reactor 1 {PFA, Coils reactor, 3.175 (⅛″) mm, 1 mL, 50° C.}. Solution 2 was also pumped {1 mL/min} to flow reactor 1 {PFA, Coils reactor, 3.175 (⅛″) mm, 1 mL, 50° C.}. The residence time of the flow reactor was 30 min. The mixture was collected with a bottle and quenched with 10 mL sat. NH₄Cl at 0° C. The mixture was extracted with DCM (20 mL×2). The separated organic layers were combined and washed with brine (20 mL×2), dried over Na₂SO₄, and filtered. The filtrate was concentrated under reduced pressure to give the crude product. The residue was purified by flash silica gel chromatography (hexanes to EtOAc/hexanes=30/70) to give 4-chloro-6-(3-(difluoromethyl)-5-fluorobenzyl)pyrimidine (105 mg, 381.25 μmol, 33.52% yield) as colorless oil. LCMS [M+H]⁺=273.1.

### Intermediate 90: Synthesis of (R)-5-((benzyloxy)methyl)-1-methyl-6,7-dihydroimidazo[1,5-a]pyrazin-8(5H)-one

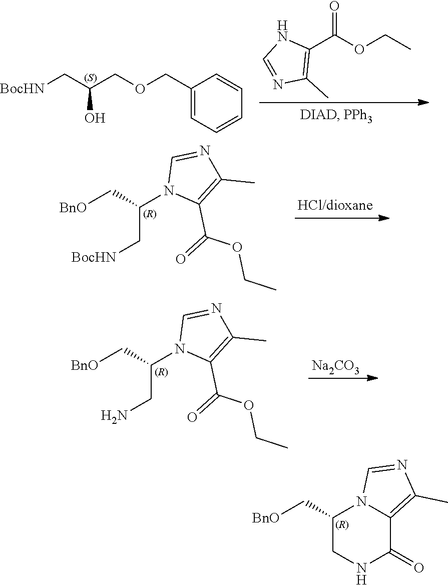

Step A: To a solution of tert-butyl(S)-(3-(benzyloxy)-2-hydroxypropyl)carbamate (602.24 mg, 2.14 mmol, 1.1 eq.) in THF (10 mL) was added DIAD (590.23 mg, 2.92 mmol, 565.90 μL, 1.5 eq.), PPh₃ (765.59 mg, 2.92 mmol, 1.5 eq.) and ethyl 4-methyl-1H-imidazole-5-carboxylate (300 mg, 1.95 mmol, 1 eq.) at 0° C., the mixture was stirred at 70° C. for 12 h. The reaction mixture (2 batches in parallel) was diluted with EtOAc (120 mL), washed by water (100 mL) and brine (100 mL). The organic layer was dried over anhydrous sodium sulfate and concentrated under vacuum to obtain the crude product. The residue was purified by flash silica gel chromatography (hexane to hexanes/EtOAc=40/60) to give ethyl (R)-1-(1-(benzyloxy)-3-((tert-butoxycarbonyl)amino) propan-2-yl)-4-methyl-1H-imidazole-5-carboxylate (2.7 g, 2.00 mmol, 51.51% yield) as a yellow solid. LCMS [M+H]⁺=418.3.

Step B: To a solution of ethyl (R)-1-(1-(benzyloxy)-3-((tert-butoxycarbonyl)amino) propan-2-yl)-4-methyl-1H-imidazole-5-carboxylate (2.7 g, 2.00 mmol, 1 eq.) in HCl/dioxane (2 M, 30 mL, 29.93 eq.) was stirred at 25° C. for 2 h. The reaction mixture was concentrated under vacuum, then diluted with H₂O (50 mL) and extracted with EtOAc (50 mL). The aqueous layer was adjusted to pH=8 with aq. NaHCO₃. The solution was extracted with EtOAc (30 mL×4). The combined organic layer was dried over Na₂SO₄, filtered and concentrated in vacuum to give ethyl (R)-1-(1-amino-3-(benzyloxy) propan-2-yl)-4-methyl-1H-imidazole-5-carboxylate (350 mg, crude) as light yellow oil: ¹HNMR (400 MHz, CDCl₃) δ=7.75 (s, 1H), 7.32-7.20 (m, 5H), 5.26-5.12 (m, 1H), 4.44 (d, J=6.0 Hz, 2H), 4.28-4.18 (m, 2H), 3.79 (dd, J=4.8, 10.4 Hz, 1H), 3.69-3.64 (m, 1H), 3.08 (br d, J=5.2 Hz, 2H), 2.42 (s, 3H), 1.30 (t, J=7.2 Hz, 3H).

Step C: To a solution of ethyl (R)-1-(1-amino-3-(benzyloxy) propan-2-yl)-4-methyl-1H-imidazole-5-carboxylate (350 mg, 1.10 mmol, 1 eq.) in MeOH (5 mL) was added Na₂CO₃ (584.41 mg, 5.51 mmol, 5 eq.). Then the mixture was stirred at 60° C. for 12 h. The reaction mixture was filtrated and concentrated under vacuum. The residue was purified by flash silica gel chromatography (DCM to DCM/MeOH=90/10) to give (R)-5-((benzyloxy)methyl)-1-methyl-6,7-dihydroimidazo[1,5-a]pyrazin-8(5H)-one (200 mg, 737.15 μmol, 66.85% yield) as a white solid: 1HNMR (400 MHz, CDCl₃) δ=7.60 (s, 1H), 7.42-7.32 (m, 3H), 7.30 (d, J=1.2 Hz, 1H), 7.28-7.27 (m, 1H), 6.00 (br s, 1H), 4.63-4.50 (m, 2H), 4.50-4.40 (m, 1H), 3.81-3.73 (m, 2H), 3.72-3.66 (m, 1H), 3.59 (td, J=4.0, 12.8 Hz, 1H), 2.56 (s, 3H).

### Intermediate 91: Synthesis of 1-ethyl-3-(2-hydroxyethyl)-6,7-dihydroimidazo[1,5-a]pyrazin-8(5H)-one

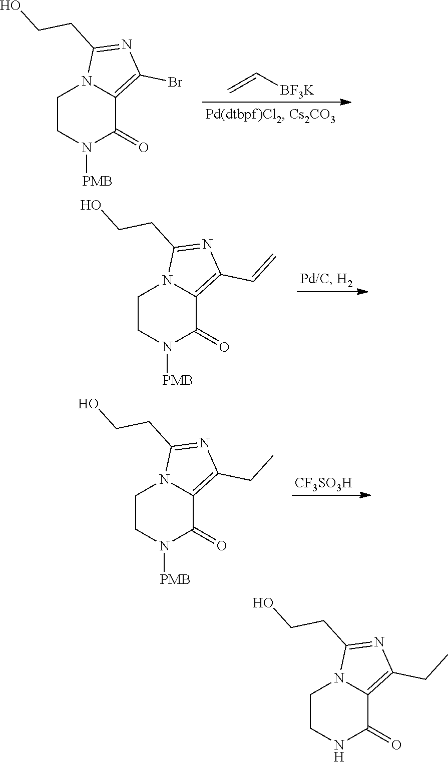

Step A: A mixture of 1-bromo-3-(2-hydroxyethyl)-7-(4-methoxybenzyl)-6,7-dihydroimidazo[1,5-a]pyrazin-8(5H)-one (270 mg, 710.09 μmol, 1 eq.), potassium trifluoro (vinyl)-borate (285.35 mg, 2.13 mmol, 3 eq.), Pd(dtbpf)Cl₂ (92.56 mg, 142.02 μmol, 0.2 eq.) and Cs₂CO₃ (694.08 mg, 2.13 mmol, 3 eq.) in toluene (7.5 mL) and H₂O (1.5 mL) was degassed and purged with N₂ three times, then the mixture was stirred at 100° C. for 12 h under N₂ atmosphere. The reaction mixture was poured into water (20 mL) and extracted with EtOAc (30 mL×3). The combined organic layers were washed with water (30 mL×3), brine (30 mL×3) and dried over Na₂SO₄, filtered and concentrated under reduced pressure. The residue was purified by flash silica gel chromatography (DCM to DCM/MeOH=95/5, TLC: DCM/MeOH=10/1) to give 3-(2-hydroxyethyl)-7-(4-methoxybenzyl)-1-vinyl-6,7-dihydroimidazo[1,5-a]pyrazin-8(5H)-one (150 mg, 458.19 μmol, 64.53% yield) as a yellow gum: ¹H NMR (400 MHz, DMSO-d₆) δ=7.33-7.18 (m, 3H), 6.93-6.87 (m, 2H), 5.94 (dd, J=2.4, 17.6 Hz, 1H), 5.21 (dd, J=2.8, 11.2 Hz, 1H), 4.83-4.71 (m, 1H), 4.58 (s, 2H), 4.17-4.03 (m, 2H), 3.73 (s, 3H), 3.72-3.65 (m, 2H), 3.54-3.48 (m, 2H), 2.79 (t, J=6.5 Hz, 2H).

Step B: To a solution of 3-(2-hydroxyethyl)-7-(4-methoxybenzyl)-1-vinyl-6,7-dihydroimidazo[1,5-a]pyrazin-8(5H)-one (150 mg, 458.19 μmol, 1 eq.) in MeOH (5 mL) was added Pd/C (97.52 mg, 91.64 μmol, 10% purity, 0.2 eq.) under N₂. The suspension was degassed under vacuum and purged with H₂ several times. The mixture was stirred under H₂ (15 psi) at 25° C. for 3 h. The reaction mixture was filtered and concentrated under reduced pressure to give 1-ethyl-3-(2-hydroxyethyl)-7-(4-methoxybenzyl)-6,7-dihydroimidazo[1,5-a]pyrazin-8(5H)-one (110 mg, crude) as a brown solid: ¹H NMR (400 MHz, DMSO-d₆) δ=7.22 (d, J=8.4 Hz, 2H), 6.90 (d, J=8.4 Hz, 2H), 4.79 (t, J=5.4 Hz, 1H), 4.55 (s, 2H), 4.05 (t, J=5.6 Hz, 2H), 3.73 (s, 3H), 3.68-3.61 (m, 2H), 3.56-3.43 (m, 2H), 3.31 (s, 2H), 2.77 (dd, J=6.8, 14.0 Hz, 2H), 1.17-1.11 (m, 3H).

Step C: To a solution of 1-ethyl-3-(2-hydroxyethyl)-7-(4-methoxybenzyl)-6,7-dihydroimidazo[1,5-a]pyrazin-8(5H)-one (100 mg, 303.59 μmol, 1 eq.) in toluene (5 mL) was added CF₃SO₃H (455.62 mg, 3.04 mmol, 268.64 μL, 10 eq.) at 0° C. The reaction mixture was stirred at 25° C. for 12 h. The reaction mixture was adjusted to pH=7 with sat. aq. NaHCO₃ and washed with EtOAc (30 mL×3). The aqueous phase was purified by reversed-phase HPLC (H₂O-MeCN condition) to give 1-ethyl-3-(2-hydroxyethyl)-6,7-dihydroimidazo[1,5-a]pyrazin-8(5H)-one (28 mg, 133.81 μmol, 44.08% yield) as a white solid: ¹H NMR (500 MHz, DMSO-d₆) δ=7.62 (s, 1H), 4.79 (br.s, 1H), 4.02 (t, J=5.5 Hz, 2H), 3.67 (t, J=6.5 Hz, 2H), 3.46-3.40 (m, 2H), 2.79-2.70 (m, 4H), 1.11 (t, J=7.5 Hz, 3H).

### Intermediate 92: Synthesis of 3-bromo-1-cyclopropyl-6,7-dihydroimidazo[1,5-a]pyrazin-8(5H)-one

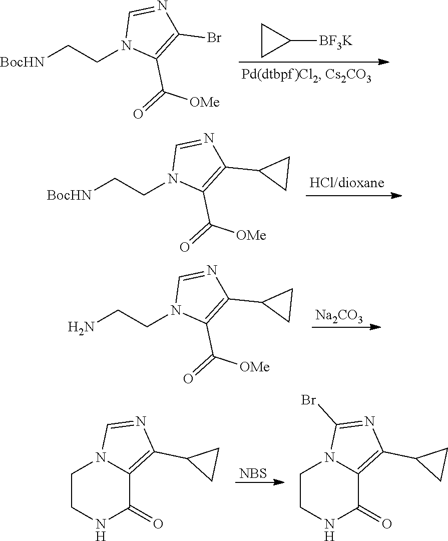

Step A: A mixture of methyl 4-bromo-1-(2-((tert-butoxycarbonyl)amino)ethyl)-1H-imidazole-5-carboxylate (5 g, 12.49 mmol, 1 eq.), potassium cyclopropyltrifluoro-borate (5.55 g, 37.48 mmol, 3 eq.), Cs₂CO₃ (12.21 g, 37.48 mmol, 3 eq.), Pd (dtbpf) C12 (814.23 mg, 1.25 mmol, 0.1 eq.) and H₂O (30 mL) in toluene (150 mL) was degassed and purged with N₂ three times, then the mixture was stirred at 110° C. for 14 h under a N₂ atmosphere. The reaction mixture was filtered and concentrated under reduced pressure to give a residue. The residue was purified by flash silica gel chromatography (hexanes to hexanes/EtOAc=55/45, TLC: hexanes/EtOAc=1/1, R_{f}=0.5) to yield methyl 1-(2-((tert-butoxycarbonyl)amino)ethyl)-4-cyclopropyl-1H-imidazole-5-carboxylate (2.8 g, 9.05 mmol, 72.45% yield) as red oil. LCMS [M+H]⁺=310.2. ¹H NMR (400 MHz, CDCl₃) δ=7.36 (s, 1H), 4.67 (s, 1H), 4.42-4.30 (m, 2H), 3.90 (s, 3H), 3.47-3.40 (m, 2H), 2.61-2.52 (m, 1H), 1.42 (s, 9H), 1.05-0.99 (m, 2H), 0.98-0.92 (m, 2H).

Step B: To a solution of methyl 1-(2-((tert-butoxycarbonyl)amino)ethyl)-4-cyclopropyl-1H-imidazole-5-carboxylate (2.8 g, 9.05 mmol, 1 eq.) in MeOH (8 mL) was added HCl/dioxane (28 mL) and the mixture was stirred at 25° C. for 2 h. The reaction mixture was concentrated under reduced pressure to yield methyl 1-(2-aminoethyl)-4-cyclopropyl-1H-imidazole-5-carboxylate (2.4 g, crude, 2HCl salt) as a yellow solid. LCMS [M+H]⁺=210.1.

Step C: To a solution of methyl 1-(2-aminoethyl)-4-cyclopropyl-1H-imidazole-5-carboxylate (2.4 g, 8.51 mmol, 1 eq, 2HCl) in MeOH (80 mL) was added Na₂CO₃ (4.51 g, 42.53 mmol, 5 eq.). The mixture was stirred at 80° C. for 14 h. The reaction mixture was filtered and concentrated under reduced pressure to give a residue. The residue was purified by flash silica gel chromatography (DCM to DCM/MeOH=93/7, TLC: DCM/MeOH=10/1, R_{f}=0.4) to yield 1-cyclopropyl-6,7-dihydroimidazo[1,5-a]pyrazin-8(5H)-one (1.15 g, 6.49 mmol, 76.30% yield) as a yellow solid: ¹H NMR (400 MHz, DMSO-d₆) δ=7.72 (br s, 1H), 7.59 (s, 1H), 4.11-4.08 (m, 2H), 3.47-3.41 (m, 2H), 2.59-2.52 (m, 1H), 0.86-0.75 (m, 4H).

Step D: To a solution of 1-cyclopropyl-6,7-dihydroimidazo[1,5-a]pyrazin-8(5H)-one (900 mg, 5.08 mmol, 1 eq.) in DCM (36 mL) was added NBS (1.18 g, 6.60 mmol, 1.3 eq.). The mixture was stirred at 25° C. for 14 h. The reaction mixture was quenched by the addition of H₂O (40 mL) and extracted with DCM (40 mL×3). The combined organic layers were concentrated under reduced pressure to give a residue. The residue was purified by flash silica gel chromatography (DCM to DCM/MeOH=97/3, TLC: DCM/MeOH=10/1, Rf=0.5) to yield 3-bromo-1-cyclopropyl-6,7-dihydroimidazo[1,5-a]pyrazin-8(5H)-one (1.1 g, 3.87 mmol, 76.11% yield) as a yellow solid: ¹H NMR (400 MHz, DMSO-d₆) δ=7.86 (s, 1H), 4.03-3.94 (m, 2H), 3.51-3.45 (m, 2H), 2.61-2.56 (m, 1H), 0.89-0.83 (m, 2H), 0.80-0.74 (m, 2H).

### Intermediate 93: Synthesis of 3-(2-hydroxyethyl)-1-methyl-5,6,7,8-tetrahydro-9H-imidazo[1,5-a][1,4]diazepin-9-one

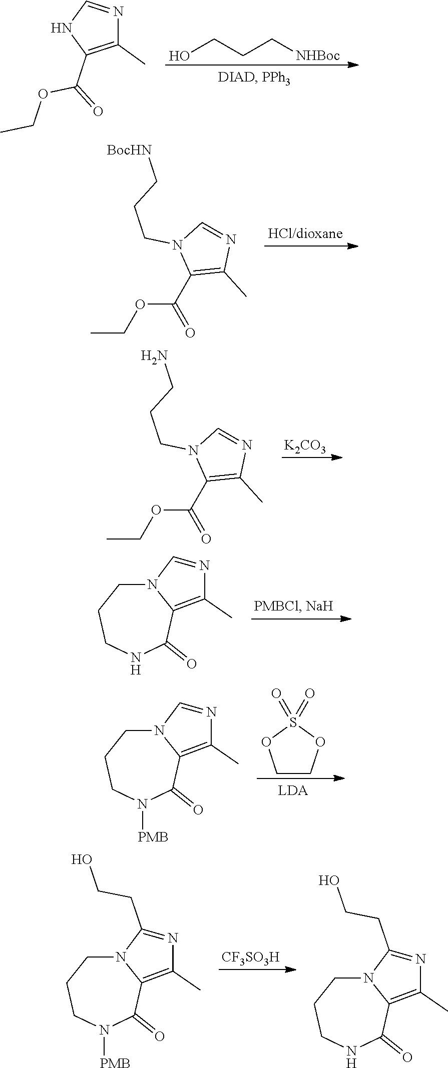

Step A: To a solution of ethyl 4-methyl-1H-imidazole-5-carboxylate (500 mg, 3.24 mmol, 1 eq.) and tert-butyl (3-hydroxypropyl)carbamate (738.79 mg, 4.22 mmol, 720.77 μL, 1.3 eq.) in THF (20 mL) was added PPh₃ (1.28 g, 4.86 mmol, 1.5 eq.) and DIAD (983.72 mg, 4.86 mmol, 943.16 μL, 1.5 eq.) at −60° C. The mixture was stirred at 20° C. for 2 h. Then, the reaction mixture was diluted with EtOAc (20 mL×3), washed by water (10 mL) and brine (10 mL). The organic layer was dried over anhydrous sodium sulfate and concentrated under vacuum. The residue was purified by flash silica gel chromatography (DCM to DCM/MeOH =96/4) to yield ethyl 1-(3-((tert-butoxycarbonyl)amino)propyl)-4-methyl-1H-imidazole-5-carboxylate (800 mg, 2.31 mmol, 71.30% yield) as colorless oil. LCMS [M+H]⁺=312.1; ¹H NMR (400 MHz, DMSO-d₆) δ=7.80 (s, 1H), 6.89 (br t, J=5.2 Hz, 1H), 4.29-4.21 (m, 2H), 4.21-4.13 (m, 2H), 2.95-2.81 (m, 2H), 2.35 (s, 3H), 1.83-1.70 (m, 2H), 1.37 (s, 9H), 1.32-1.27 (m, 3H).

Step B: A mixture of ethyl 1-(3-((tert-butoxycarbonyl)amino)propyl)-4-methyl-1H-imidazole-5-carboxylate (4 g, 12.85 mmol, 1 eq.) in HCl/dioxane (40 mL) was stirred at 25° C. for 3 h. The mixture was concentrated under reduced pressure to give ethyl 1-(3-aminopropyl)-4-methyl-1H-imidazole-5-carboxylate (3.18 g, crude, HCl salt) as a white solid, which was used in the next step without further purification. LCMS [M+H]⁺=212.1.

Step C: To a solution of ethyl 1-(3-aminopropyl)-4-methyl-1H-imidazole-5-carboxylate (3.18 g, 12.84 mmol, 1 eq, HCl) in MeOH (50 mL) was added K₂CO₃ (8.87 g, 64.19 mmol, 5 eq.). The mixture was stirred at 80° C. for 16 h. The reaction mixture was filtered and concentrated under vacuum to dryness to give a crude product. The residue was purified by flash silica gel chromatography (DCM to DCM/MeOH=91/9) to yield 1-methyl-5,6,7,8-tetrahydro-9H-imidazo[1,5-a][1,4]diazepin-9-one (1.76 g, 10.65 mmol, 83.00% yield) as a white solid: ¹H NMR (400 MHz, DMSO-d₆) δ=7.72 (br s, 1H), 7.59 (s, 1H), 4.14 (t, J=6.8 Hz, 2H), 3.05 (q, J=6.4 Hz, 2H), 2.22 (s, 3H), 2.00-1.90 (m, 2H).

Step D: To a solution of 1-methyl-5,6,7,8-tetrahydro-9H-imidazo[1,5-a][1,4]diazepin-9-one (1.76 g, 10.65 mmol, 1 eq.) in DMF (20 mL) was added NaH (639.19 mg, 15.98 mmol, 42.37 μL, 60% purity, 1.5 eq.) at 0° C. under a N₂ atmosphere. The mixture was stirred at 0° C. for 0.5 h. Then, PMBCl (2.50 g, 15.98 mmol, 2.17 mL, 1.5 eq.) was added into the mixture at 0° C. and the mixture was stirred at 25° C. for 2.5 h. The reaction mixture was quenched by the addition of NH₄Cl (10 mL) at 0° C., and then extracted with DCM (10 mL×3). The combined organic layers were dried over Na₂SO₄, filtered, and concentrated under reduced pressure to give a residue. The residue was purified by flash silica gel chromatography (DCM to DCM/MeOH=96/4) to yield 8-(4-methoxybenzyl)-1-methyl-5,6,7,8-tetrahydro-9H-imidazo[1,5-a][1,4]diazepin-9-one (3 g, 10.20 mmol, 95.72% yield) as a yellow solid. LCMS [M+H]⁺=286.1; ¹H NMR (400 MHz, DMSO-d₆) δ=7.57 (s, 1H), 7.30 (d, J=8.4 Hz, 2H), 6.91 (d, J=8.4 Hz, 2H), 4.54 (s, 2H), 4.04 (t, J=6.8 Hz, 2H), 3.74 (s, 3H), 3.18 (t, J=6.4 Hz, 2H), 2.24 (s, 3H), 1.87-1.66 (m, 2H).

Step E: A solution of 8-(4-methoxybenzyl)-1-methyl-5,6,7,8-tetrahydro-9H-imidazo[1,5-a][1,4]diazepin-9-one (1 g, 3.50 mmol, 1 eq.) in THF (30 mL) was prepared. A second solution of LDA (2 M, 3.15 mL, 1.8 eq.) in THF was also prepared. A third solution of 1,2-ethylene sulfate (1.09 g, 8.76 mmol, 2.5 eq.) in THF (18.75 mL) was prepared. The solution of 8-(4-methoxybenzyl)-1-methyl-5,6,7,8-tetrahydro-9H-imidazo[1,5-a][1,4]diazepin-9-one was pumped {18.727 mL/min} to flow reactor 1 {PFA, Coils reactor, 1.590 ( 1/16″) mm, 0.540 mL, −30.0° C.}. The solution of LDA was pumped {2.873 mL/min} to flow reactor 1 {Coils reactor, 1.590 ( 1/16″) mm, 0.540 mL, −30.0° C.}. The solution of 1,2-ethylene sulfate was pumped {11.941 mL/min} to flow reactor 2 {PFA, Coils reactor, 1.590 ( 1/16″) mm, 0.540 mL, −30.0° C.} which was on the outlet of flow reactor 1. The residence time of flow reactor 1 was 0.025 min. The residence time of flow reactor 2 was 0.016 min. The output mixture was collected into a bottle and quenched by 6.0 mL aq. HCl at 0.0° C. To this mixture was added aq. NaHCO₃20 mL at 0° C. Then, the organics were extracted with EtOAc (20 mL×3). The combined organic layers were dried over Na₂SO₄, filtered and concentrated under reduced pressure to give a residue. The residue was purified by flash silica gel chromatography (DCM to DCM/MeOH=96/4) to yield 3-(2-hydroxyethyl)-8-(4-methoxybenzyl)-1-methyl-5,6,7,8-tetrahydro-9H-imidazo[1,5-a][1,4]diazepin-9-one (570 mg, 1.71 mmol, 48.88% yield) as a yellow solid. LCMS [M+H]⁺=330.2; ¹H NMR (400 MHz, DMSO-d₆) δ=7.29 (d, J=8.4 Hz, 2H), 6.91 (d, J=8.8 Hz, 2H), 4.75 (t, J=5.6 Hz, 1H), 4.53 (s, 2H), 3.99-3.86 (m, 2H), 3.74 (s, 3H), 3.64 (td, J=6.4, 12.0 Hz, 2H), 3.17 (br t, J=6.4 Hz, 2H), 2.78-2.61 (m, 2H), 2.20 (s, 3H), 1.75 (br t, J=6.4 Hz, 2H).

Step F: To a solution of 3-(2-hydroxyethyl)-8-(4-methoxybenzyl)-1-methyl-5,6,7,8-tetrahydro-9H-imidazo[1,5-a][1,4]diazepin-9-one (270 mg, 819.69 μmol, 1 eq.) in toluene (2 mL) was added TfOH (1.23 g, 8.20 mmol, 725.33 μL, 10 eq.) at 0° C. The mixture was stirred at 25° C. for 3 h. Aq. NaHCO₃ was added into the mixture to change the pH=8, then the mixture (combined with 300 mg batch) was diluted with EtOAc (2 mL×2), washed by water (4 mL×3). The aqueous phase was collected for purification. The crude product was purified by reversed-phase HPLC (MeCN/Water=1% to 5%) to yield 3-(2-hydroxyethyl)-1-methyl-5,6,7,8-tetrahydro-9H-imidazo[1,5-a][1,4]diazepin-9-one (350 mg) as a white solid. LCMS [M+H]⁺=210.2; ¹H NMR (400 MHz, DMSO-d₆) δ=7.67 (br t, J=4.8 Hz, 1H), 5.37-4.48 (m, 1H), 4.07-4.00 (m, 2H), 3.69-3.64 (m, 2H), 3.02 (q, J=6.0 Hz, 2H), 2.80-2.74 (m, 2H), 2.19-2.17 (m, 3H), 1.96-1.87 (m, 2H).

### Intermediate 94: Synthesis of methyl 1-ethyl-8-oxo-5,6,7,8-tetrahydroimidazo[1,5-a]pyrazine-3-carboxylate

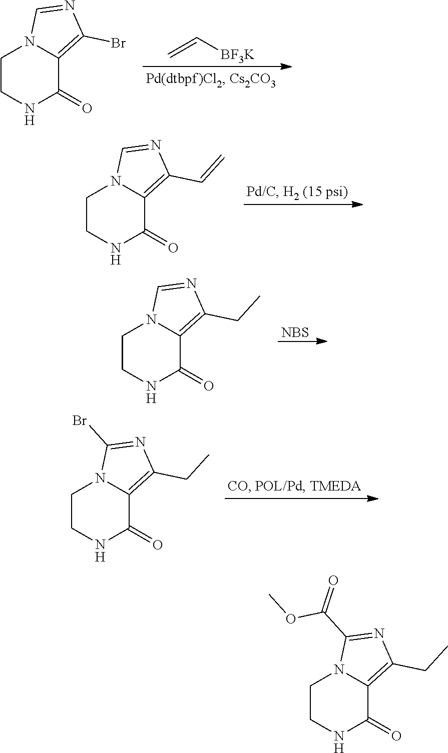

Step A: To a solution of 1-bromo-6,7-dihydroimidazo[1,5-a]pyrazin-8(5H)-one (3.00 g, 13.89 mmol, 1 eq.) and potassium trifluoro (vinyl)-borate (5.58 g, 41.66 mmol, 3 eq.) in toluene (54 mL) and H₂O (18 mL) was added Cs₂CO₃ (13.57 g, 41.66 mmol, 3 eq.) and Pd(dtbpf)Cl₂ (724.04 mg, 1.11 mmol, 0.08 eq.). The mixture was stirred at 100° C. for 18 h under N₂. The mixture was concentrated under vacuum to give a residue. The residue was purified by flash silica gel chromatography (DCM to DCM/MeOH=95/5, TLC: DCM/MeOH=10/1, R_{f}=0.4) to give 1-vinyl-6,7-dihydroimidazo[1,5-a]pyrazin-8(5H)-one (2 g, 11.03 mmol, 79.44% yield) as a brown solid. LCMS [M+H]⁺=164.1; ¹H NMR (400 MHz, DMSO-d₆) δ=7.92 (s, 1H), 7.77 (s, 1H), 7.16 (dd, J=10.8, 17.6 Hz, 1H), 5.97 (dd, J=2.4, 17.6 Hz, 1H), 5.21 (dd, J=2.4, 11.2 Hz, 1H), 4.18-4.12 (m, 2H), 3.50-3.44 (m, 2H).

Step B: To a solution of 1-vinyl-6,7-dihydroimidazo[1,5-a]pyrazin-8(5H)-one (2.25 g, 13.79 mmol, 1 eq.) in MeOH (50 mL) was added Pd/C (733.70 mg, 689.44 μmol, 10% purity, 0.05 eq.) under an argon atmosphere. The suspension was degassed and purged with H₂. The mixture was stirred under H₂ (15 Psi) at 25° C. for 5 h. The reaction mixture was filtered on celite and concentrated under reduced pressure. The residue was purified by flash silica gel chromatography (DCM to DCM/MeOH=95/5, TLC: DCM/MeOH=10/1, R_{f}=0.4) to give 1-ethyl-6,7-dihydroimidazo[1,5-a]pyrazin-8(5H)-one (1.9 g, 11.50 mmol, 83.41% yield) as a brown solid: ¹H NMR (400 MHz, DMSO-d₆) δ=7.72 (s, 1H), 7.66 (s, 1H), 4.11 (t, J=5.6 Hz, 2H), 3.51-3.40 (m, 2H), 2.76 (q, J=7.6 Hz, 2H), 1.12 (t, J=7.6 Hz, 3H).

Step C: A solution of 1-ethyl-6,7-dihydroimidazo[1,5-a]pyrazin-8(5H)-one (200 mg, 1.21 mmol, 1 eq.) in MeCN (5 mL) was added NBS (323.23 mg, 1.82 mmol, 1.5 eq.). The mixture was stirred at 25° C. for 8 h. The mixture was diluted with H₂O (20 mL) and extracted with DCM/i-PrOH=3/1 (30 mL×3). The combined organic layers were washed with brine (20 mL×2), dried over anhydrous Na₂SO₄, filtered, and concentrated under reduced pressure to give a residue. The residue was purified by flash silica gel chromatography (DCM to DCM/MeOH=95/5, TLC: DCM/MeOH=10/1, R_{f}=0.4) to give a 3-bromo-1-ethyl-6,7-dihydroimidazo[1,5-a]pyrazin-8(5H)-one (110 mg, 450.66 μmol, 37.22% yield) as a yellow solid: ¹H NMR (400 MHz, DMSO-d₆) δ=7.88 (s, 1H), 4.03-3.98 (m, 2H), 3.51-3.46 (m, 2H), 2.77 (q, J=7.6 Hz, 2H), 1.12 (t, J=7.6 Hz, 3H).

Step D: A solution of 3-bromo-1-ethyl-6,7-dihydroimidazo[1,5-a]pyrazin-8(5H)-one (100 mg, 409.69 μmol, 1 eq.) and TMEDA (142.82 mg, 1.23 mmol, 185.48 L, 3 eq.) in MeOH (2 mL) was prepared. The fixed bed (volume 50 mL) was completely packed with granular catalyst (WXPS1001 (3% Pd@POL), 17 g, WuXi LabNetwork, Shanghai, China; see also, CN application No. 2025109971019). The CO back pressure regulator was adjusted to 3 Mpa, and the CO flow rate was {1.500 eq, 20.600 sccm}. The reaction solution was pumped {3 mL/min} through the fixed bed {12.700 (½″) mm, 50.000 mL, 140.0° C.}. The reaction mixture was continuously collected from the reactor outlet into the container for 40 min. The mixture was concentrated under reduced pressure to give methyl 1-ethyl-8-oxo-5,6,7,8-tetrahydroimidazo[1,5-a]pyrazine-3-carboxylate (90 mg, crude) as a yellow solid. LCMS [M+H]⁺=224.1.

Intermediate 95: Synthesis of ethyl 3-bromo-4-oxo-4,5,6,7-tetrahydropyrazolo[1,5-a]pyrazine-2-carboxylate

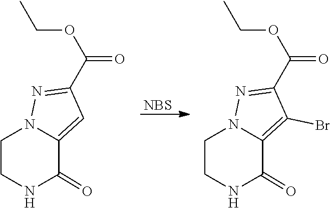

Step A: To a solution of ethyl 4-oxo-4,5,6,7-tetrahydropyrazolo[1,5-a]pyrazine-2-carboxylate (500 mg, 2.40 mmol, 1 eq.) in MeCN (10 mL) was added NBS (510.46 mg, 2.86 mmol, 1.2 eq.), the mixture was stirred at 80° C. for 4 h under a N₂ atmosphere. The reaction mixture was concentrated under vacuum. The residue was purified by prep-HPLC (column: 40-WePure Biotech XP tC18 150×30 mm, 7 μm; mobile phase: [H₂O (0.05% NH₃H₂O+10 mM NH₄HCO₃)-ACN]; gradient: 44%-74% ACN over 10.0 min) to yield ethyl 3-bromo-4-oxo-4,5,6,7-tetrahydropyrazolo[1,5-a]pyrazine-2-carboxylate (140 mg, 485.95 μmol, 20.24% yield) as a white solid: ¹H NMR (400 MHz, DMSO-d₆) δ=8.44 (br s, 1H), 4.46-4.37 (m, 2H), 4.30 (q, J=7.2 Hz, 2H), 3.68-3.57 (m, 2H), 1.30 (t, J=6.8 Hz, 3H).

### Intermediate 96: Synthesis of 3-(((tert-butyldimethylsilyl)oxy)methyl)-1-ethyl-6,7-dihydroimidazo[1,5-a]pyrazin-8 (5)-one

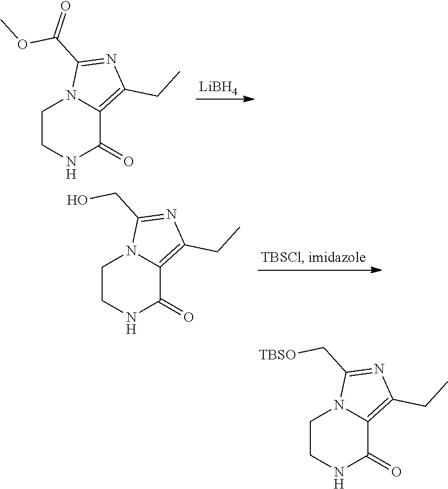

Step A: To a solution of methyl 1-ethyl-8-oxo-5,6,7,8-tetrahydroimidazo[1,5-a]pyrazine-3-carboxylate (230 mg, 1.03 mmol, 1 eq.) in THF (20 mL) was added LiBH₄ (2 M, 2.58 mL, 5 eq.) at 0° C. The mixture was stirred at 0° C. for 0.5 h under a N₂ atmosphere. The reaction mixture was quenched with H₂O (3 mL) at 0° C. dropwise and then concentrated under reduced pressure to remove THF. The residue was purified by reversed-phase silica gel column (MeCN/H₂O) to give 1-ethyl-3-(hydroxymethyl)-6,7-dihydroimidazo[1,5-a]pyrazin-8(5H)-one (150 mg, 768.37 μmol, 74.57% yield) as a yellow solid. LCMS [M+H]⁺=196.3; ¹H NMR (400 MHz, DMSO-d₆) δ=7.83 (s, 1H), 5.52 (s, 1H), 4.51 (s, 2H), 4.16-4.06 (m, 2H), 3.48-3.44 (m, 2H), 2.78 (q, J=7.6 Hz, 2H), 1.13 (t, J=7.6 Hz, 3H).

Step B: To a solution of 1-ethyl-3-(hydroxymethyl)-6,7-dihydroimidazo[1,5-a]pyrazin-8(5H)-one (150 mg, 768.37 μmol, 1 eq.) and imidazole (313.85 mg, 4.61 mmol, 6 eq.) in DMF (2 mL) and THF (2 mL) was added TBSCl (347.43 mg, 2.31 mmol, 283.62 μL, 3 eq.) in THF (0.5 mL) at 0° C. The mixture was stirred at 25° C. for 1 h under N₂. The mixture was diluted with H₂O (20 mL) and extracted with EtOAc/THF=2/1 (20 mL×3). The combined organic layers were dried over anhydrous Na₂SO₄, filtered, and concentrated under reduced pressure to give a residue. The residue was purified by flash silica gel chromatography (DCM to DCM/MeOH=98/2, TLC: DCM/MeOH=10/1, R_{f}=0.3) to give 3-(((tert-butyldimethylsilyl)oxy)methyl)-1-ethyl-6,7-dihydroimidazo[1,5-a]pyrazin-8(5H)-one (73 mg, 235.88 μmol, 30.69% yield) as a yellow solid. LCMS [M+H]⁺=310.3.

### Intermediate 97: Synthesis of 1-ethyl-3-(2-hydroxyethoxy)-6,7-dihydroimidazo[1,5-a]pyrazin-8(5H)-one

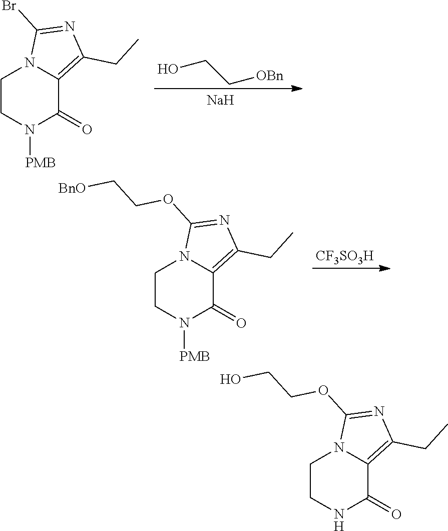

Step A: To a solution of 3-bromo-1-ethyl-7-(4-methoxybenzyl)-6,7-dihydroimidazo[1,5-a]pyrazin-8(5H)-one (935.95 mg, 6.15 mmol, 873.90 μL, 4 eq.) in DMF (10 mL) was added NaH (368.99 mg, 9.22 mmol, 60% purity, 6 eq.) under a N₂ atmosphere, the mixture was stirred at 0° C. for 0.5 h. Then 2-(benzyloxy) ethan-1-ol (700 mg, 1.54 mmol, 1 eq.) was added to the above mixture reaction in one portion. The mixture was stirred at 60° C. for 10 h under N₂ atmosphere. Water (15 mL) was added slowly into the above mixture at 0° C. The mixture was extracted with EtOAc (20 mL×2). The separated organic layers were combined and washed with brine (20 mL×2), dried over Na₂SO₄, filtered, and concentrated under reduced pressure to give the crude product, which was purified by flash silica gel chromatography (hexane to hexane/EtOAc=10/90) to give 3-(2-(benzyloxy) ethoxy)-1-ethyl-7-(4-methoxybenzyl)-6,7-dihydroimidazo[1,5-a]pyrazin-8(5H)-one (430 mg, 960.68 μmol, 62.48% yield) as yellow oil. LCMS [M+H]⁺=436.3; ¹H NMR (400 MHz, DMSO-d₆) δ=7.37-7.15 (m, 7H), 6.93-6.85 (m, 2H), 4.56-4.49 (m, 4H), 4.49-4.44 (m, 2H), 3.83 (t, J=5.6 Hz, 2H), 3.77-3.70 (m, 5H), 3.44 (t, J=5.6 Hz, 2H), 2.76 (q, J=7.6 Hz, 2H), 1.12 (t, J=7.6 Hz, 3H).

Step B: To a solution of 3-(2-(benzyloxy) ethoxy)-1-ethyl-7-(4-methoxybenzyl)-6,7-dihydroimidazo[1,5-a]pyrazin-8(5H)-one (430 mg, 960.68 μmol, 1 eq.) in toluene (5 mL) was added CF₃SO₃H (1.44 g, 9.61 mmol, 850.09 μL, 10 eq.) at 0° C. under N₂. Then the mixture was stirred at 30° C. for 2 h under N₂ atmosphere. Then, sat. NaHCO₃ was added slowly into the above mixture at 0° C. until pH ~8. The mixture was extracted with EtOAc (20 mL×2). The leaving aqueous layer was purified by reversed-phase HPLC (neutral condition) [Column: C18 150*25 mm*5 μm, Condition: 18% (A: H₂O, B: CH₃CN), flow rate: 40 mL/min] to give 1-ethyl-3-(2-hydroxyethoxy)-6,7-dihydroimidazo[1,5-a]pyrazin-8(5H)-one (170 mg, 754.74 μmol, 78.56% yield) as a yellow solid. LCMS [M+H]⁺=226.1; ¹H NMR (400 MHz, DMSO-d₆) δ=7.51 (s, 1H), 5.01-4.82 (m, 1H), 4.31 (t, J=4.8 Hz, 2H), 3.80 (t, J=5.6 Hz, 2H), 3.69 (t, J=4.8 Hz, 2H), 3.42-3.36 (m, 2H), 2.70 (q, J=7.6 Hz, 2H), 1.10 (t, J=7.6 Hz, 3H).

### Intermediate 98: Synthesis of 3-(2-((tert-butyldimethylsilyl)oxy)ethyl)-1-chloro-6,7-dihydroimidazo[1,5-a]pyrazin-8(5H)-one

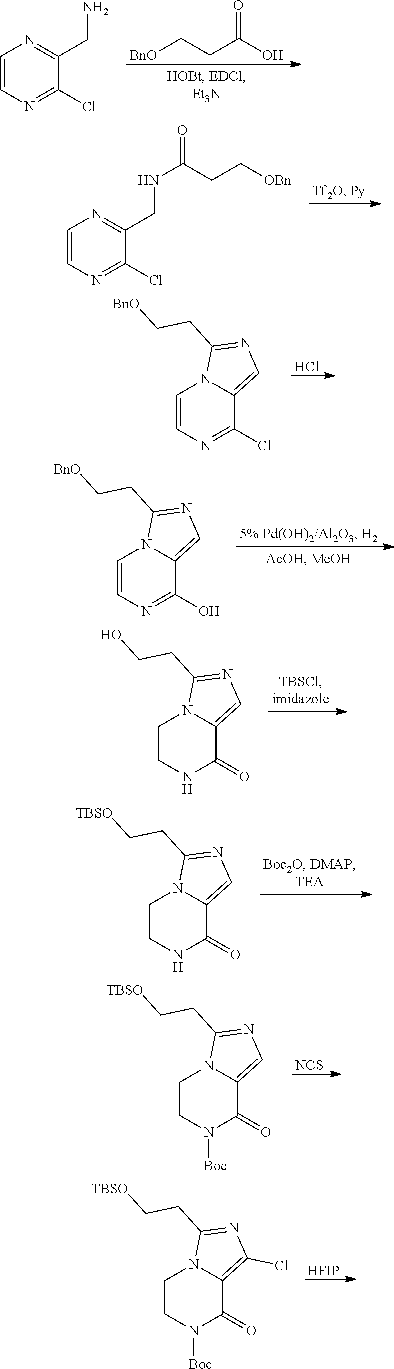

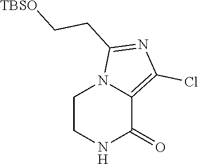 [SMILES: CC(C)(C)[Si](C)(C)OCCc1nc(Cl)c2n1CCNC2=O]

Step A: To a solution of (3-chloropyrazin-2-yl) methanamine dihydrochloride (10 g, 46.19 mmol, 1 eq.), 3-(benzyloxy) propanoic acid (9.16 g, 50.81 mmol, 1.1 eq.) in DMF (250 mL) was added EDCI (13.28 g, 69.29 mmol, 1.5 eq.), HOBt (9.36 g, 69.29 mmol, 1.5 eq.), TEA (14.02 g, 138.57 mmol, 19.29 mL, 3 eq.), the mixture was stirred at 25° C. for 12 h. Then, the reaction mixture was diluted with EtOAc (300 mL), washed successively with water (300 mL) and brine (300 mL). The organic layer was dried over anhydrous sodium sulfate and concentrated in vacuo to obtain the crude. The crude was purified by flash silica gel chromatography (hexanes to hexanes/THF=1/1, TLC: hexanes/THF=1/1, R_{f}=0.4) to yield 3-(benzyloxy)-N-((3-chloropyrazin-2-yl)methyl) propanamide (14 g, 45.79 mmol, 99.13% yield) as a white solid: ¹H NMR (400 MHz, DMSO-d₆) δ=8.60 (d, J=2.4 Hz, 1H), 8.50-8.39 (m, 2H), 7.36-7.27 (m, 5H), 4.52 (d, J=5.6 Hz, 2H), 4.45 (s, 2H), 3.66 (t, J=6.4 Hz, 2H), 2.49-2.45 (m, 2H).

Step B: To a solution of 3-(benzyloxy)-N-((3-chloropyrazin-2-yl)methyl) propanamide (14 g, 45.79 mmol, 1 eq.) in DCM (400 mL) was added pyridine (50.71 g, 641.03 mmol, 51.74 mL, 14 eq.) at 0° C. Then, Tf₂O (77.51 g, 274.73 mmol, 45.33 mL, 6 eq.) in DCM (50 mL) was added dropwise at 0° C. The resulting mixture was stirred at 0° C. for 1 h. Then, the reaction mixture was quenched by addition H₂O (500 mL) at 0° C., and adjusted pH to 8 by the addition of a saturated solution of NaHCO₃. The reaction was extracted with DCM (500 mL×3). The combined organic layers were dried over Na₂SO₄, filtered, and concentrated under vacuum to give a residue. The residue was purified by flash silica gel chromatography (hexanes to hexanes/EtOAc=1:1, TLC: Commercial hexanes/EtOAc=1:1, R_{f}=0.3) to yield 3-(2-(benzyloxy)ethyl)-8-chloroimidazo[1,5-a]pyrazine (12 g, 41.70 mmol, 91.08% yield) as yellow oil: ¹H NMR (400 MHz, DMSO-d₆) δ=8.34-8.30 (m, 1H), 7.81 (s, 1H), 7.36 (d, J=4.8 Hz, 1H), 7.32-7.13 (m, 5H), 4.46 (s, 2H), 3.85 (t, J=6.4 Hz, 2H), 3.36-3.32 (m, 2H).

Step C: To a solution of 3-(2-(benzyloxy)ethyl)-8-chloroimidazo[1,5-a]pyrazine (4.25 g, 14.77 mmol, 1 eq.) in dioxane (50 mL) was added HCl (2 M, 25 mL, 3.39 eq.). The mixture was stirred at 80° C. for 2 h. Then, the reaction mixture was adjusted pH to 8 by the addition of aq. Na₂CO₃ at 0° C., and then diluted with H₂O (50 mL) and extracted with EtOAc (100 mL×3). The combined organic layers were dried over Na₂SO₄, filtered, and concentrated under reduced pressure to give 3-(2-(benzyloxy)ethyl)imidazo[1,5-a]pyrazin-8-ol (3.7 g, crude) as a white solid: ¹H NMR (400 MHz, DMSO-d₆) δ=10.57 (br s, 1H), 7.67 (s, 1H), 7.40-7.14 (m, 6H), 6.62 (d, J=5.6 Hz, 1H), 4.48 (s, 2H), 3.81 (t, J=6.4 Hz, 2H), 3.19 (t, J=6.8 Hz, 2H).

Step D: A solution of 3-(2-(benzyloxy)ethyl)imidazo[1,5-a]pyrazin-8-ol (500.00 mg, 1.86 mmol, 1 eq.) in AcOH (2.10 g, 34.94 mmol, 2 mL, 18.82 eq.), THF (10 mL) and MeOH (10 mL) was prepared. The fixed bed (volume 10 mL) was completely packed with the granular catalyst 5% Pd(OH)₂/Al₂O₃ (2 g, 1.00 eq.). The H₂ back pressure regulator was adjusted to 1 Mpa, and the H₂ flow rate was set to: 120.500 eq, 30.100 sccm. The solution of starting material was pumped at a rate of 0.3 mL/min to the flow reactor (6.350 (¼″) mm, 5.000 mL, 70° C.). The reaction mixture was continuously collected from the reactor outlet into the container. The reaction mixture was concentrated under reduced pressure to give a residue. The residue was triturated with THF (10 mL) at 25° C. for 15 min to yield 3-(2-hydroxyethyl)-6,7-dihydroimidazo[1,5-a]pyrazin-8 (5/I)-one (300 mg, 1.57 mmol, 84.72% yield, 95%) as a white solid. LCMS [M+H]⁺=182.0; ¹H NMR (400 MHz, D₂O) δ=7.59 (s, 1H), 4.19 (t, J=6.0 Hz, 2H), 3.90 (t, J=6.0 Hz, 2H), 3.66 (t, J=6.0 Hz, 2H), 2.96 (t, J=6.0 Hz, 2H).

Step E: To a solution of 3-(2-hydroxyethyl)-6,7-dihydroimidazo[1,5-a]pyrazin-8(5H)-one (700 mg, 3.86 mmol, 1 eq.) in DMF (20 mL) was added imidazole (1.58 g, 23.18 mmol, 6 eq.) and tert-butyldimethylsilyl chloride (1.75 g, 11.59 mmol, 1.43 mL, 3 eq.). The mixture was stirred at 60° C. for 16 h. Then, the reaction mixture was diluted with H₂O (50 mL) and extracted with EtOAc/THF=1/1 (50 mL×3). The combined organic layers were dried over Na₂SO₄, filtered and concentrated under vacuum to give a residue. The residue was purified by flash silica gel chromatography (DCM to DCM/MeOH=10:1, TLC: DCM/MeOH=10/1, R_{f}=0.3) to yield 3-(2-((tert-butyldimethylsilyl)oxy)ethyl)-6,7-dihydroimidazo[1,5-a]pyrazin-8(5H)-one (520 mg, 1.76 mmol, 47.27% yield) as a white solid: ¹H NMR (400 MHz, DMSO-d₆) δ=7.81 (br s, 1H), 7.37 (s, 1H), 4.07 (t, J=5.6 Hz, 2H), 3.88 (t, J=6.4 Hz, 2H), 3.51-3.42 (m, 2H), 2.87 (t, J=6.4 Hz, 2H), 0.81 (s, 9H), −0.04 (s, 6H).

Step F: To a solution of 3-(2-((tert-butyldimethylsilyl)oxy)ethyl)-6,7-dihydroimidazo[1,5-a]pyrazin-8(5H)-one (424 mg, 1.44 mmol, 1 eq.), (Boc) 20 (939.63 mg, 4.31 mmol, 989.08 μL, 3 eq.) in DCM (15 mL) was added DMAP (35.07 mg, 287.02 μmol, 0.2 eq.) and TEA (290.43 mg, 2.87 mmol, 399.50 μL, 2 eq.). The mixture was stirred at 25° C. for 16 h. Then, the reaction mixture was concentrated under reduced pressure to give a residue. The residue was purified by flash silica gel chromatography (hexanes to hexanes/THF=1/1, TLC: hexanes/THF=1/1, R_{f}=0.4) to yield tert-butyl 3-(2-((tert-butyldimethylsilyl)oxy)ethyl)-8-oxo-5,6-dihydroimidazo[1,5-a]pyrazine-7 (8H)-carboxylate (550 mg, 1.39 mmol, 96.89% yield) as yellow oil: ¹H NMR (400 MHz, DMSO-d₆) δ=7.62 (s, 1H), 4.20-4.15 (m, 2H), 4.06-3.98 (m, 2H), 3.89 (t, J=6.4 Hz, 2H), 2.88 (t, J=6.4 Hz, 2H), 1.48 (s, 9H), 0.81 (s, 9H), −0.03 (s, 6H).

Step G: To a solution of tert-butyl 3-(2-((tert-butyldimethylsilyl)oxy)ethyl)-8-oxo-5,6-dihydroimidazo[1,5-a]pyrazine-7 (8H)-carboxylate (360 mg, 910.09 μmol, 1 eq.) in MeCN (15 mL) was added NCS (127.60 mg, 955.59 μmol, 1.05 eq.). The mixture was stirred at 70° C. for 16 h. Then, the reaction mixture was concentrated under reduced pressure to give a residue. The residue was purified by flash silica gel chromatography (hexanes to hexanes/EtOAc=1/1, TLC: hexanes/EtOAc=1/1, R_{f}=0.4) to yield tert-butyl 3-(2-((tert-butyldimethylsilyl)oxy)ethyl)-1-chloro-8-oxo-5,6-dihydroimidazo[1,5-a]pyrazine-7 (8H)-carboxylate (280 mg, 494.87 μmol, 54.38% yield) as colorless oil. LCMS [M+H]⁺=430.3; 1H NMR (400 MHz, CDCl₃) δ=4.21-4.16 (m, 2H), 4.15-4.07 (m, 2H), 3.96 (t, J=6 Hz, 2H), 2.89 (t, J=6 Hz, 2H), 1.58 (s, 9H), 0.84 (s, 9H), −0.01 (s, 6H).

Step H: A mixture of tert-butyl 3-(2-((tert-butyldimethylsilyl)oxy)ethyl)-1-chloro-8-oxo-5,6-dihydroimidazo[1,5-a]pyrazine-7 (8H)-carboxylate (280 mg, 651.14 μmol, 1 eq.) in HFIP (15 mL) was degassed and purged with N₂ for 3 times, and then the mixture was stirred at 70° C. for 2.5 h under N₂ atmosphere. The mixture was diluted with water (10 mL), the resulting mixture was extracted by DCM (10 mL×2). The combined organic layer was dried over anhydrous sodium sulfate, filtered, and concentrated under reduced pressure to yield 3-(2-((tert-butyldimethylsilyl)oxy)ethyl)-1-chloro-6,7-dihydroimidazo[1,5-a]pyrazin-8(5H)-one (170 mg, 515.31 μmol, 79.14% yield) as a white solid: ¹H NMR (400 MHz, CDCl₃) δ=6.26 (br s, 1H), 4.27-4.12 (m, 2H), 3.97 (t, J=6.0 Hz, 2H), 3.73-3.54 (m, 2H), 2.91 (t, J=6.0 Hz, 2H), 0.85 (s, 9H), 0.00 (s, 6H).

### Intermediate 99: Synthesis of (R)-7-((benzyloxy)methyl)-3-methyl-6,7-dihydropyrazolo[1,5-a]pyrazin-4 (5)-one

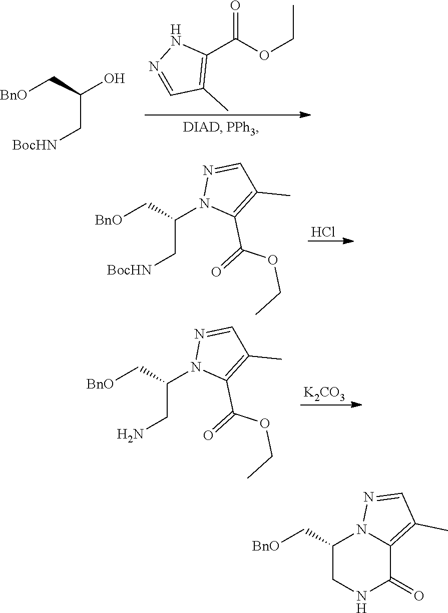

Step A: A mixture of tert-butyl(S)-(3-(benzyloxy)-2-hydroxypropyl)carbamate (2.37 g, 8.43 mmol, 1.3 eq.), ethyl 4-methyl-1H-pyrazole-5-carboxylate (1 g, 6.49 mmol, 1 eq.), DIAD (2.62 g, 12.97 mmol, 2.52 mL, 2 eq.), PPh₃ (3.40 g, 12.97 mmol, 2 eq.) in THF (20 mL) was degassed and purged with N₂ three times. Then, the mixture was stirred at 25° C. for 16 h under N₂ atmosphere. The reaction mixture was diluted with H₂O (100 mL) and extracted with EtOAc (100 mL×3). The combined organic layers were dried over Na₂SO₄, filtered, and concentrated under reduced pressure to give a residue. The residue was purified by flash silica gel chromatography (hexanes to hexanes/EtOAc=4/1, TLC: hexanes/EtOAc=2/1, R_{f}=0.8) to give ethyl (R)-1-(1-(benzyloxy)-3-((tert-butoxycarbonyl)amino) propan-2-yl)-4-methyl-1H-pyrazole-5-carboxylate (2.1 g, 5.03 mmol, 77.55% yield) as yellow oil: ¹H NMR (400 MHz, DMSO-d₆) δ=7.47 (s, 1H), 7.37-7.16 (m, 5H), 6.85 (br t, J=5.6 Hz, 1H), 5.54 (quin, J=6.4 Hz, 1H), 4.51-4.20 (m, 4H), 3.80-3.65 (m, 2H), 3.50-3.39 (m, 1H), 3.26 (br dd, J=6.4, 14.0 Hz, 1H), 2.26-2.14 (m, 3H), 1.40-1.28 (m, 12H).

Step B: To a solution of ethyl (R)-1-(1-(benzyloxy)-3-((tert-butoxycarbonyl)amino) propan-2-yl)-4-methyl-1H-pyrazole-5-carboxylate (2.1 g, 5.03 mmol, 1 eq.) in dioxane (10 mL) was added HCl (2 M, 25.15 mL, 10 eq.). The mixture was stirred at 25° C. for 16 h. The reaction mixture was concentrated under reduced pressure to give ethyl (R)-1-(1-amino-3-(benzyloxy) propan-2-yl)-4-methyl-1H-pyrazole-5-carboxylate hydrochloride (1.78 g, crude) as a white salt. LCMS [M+H]⁺=318.2.

Step C: To a solution of ethyl (R)-1-(1-amino-3-(benzyloxy) propan-2-yl)-4-methyl-1H-pyrazole-5-carboxylate hydrochloride (1.78 g, 5.03 mmol, 1 eq, HCl) in MeOH (30 mL) was added K₂CO₃ (3.48 g, 25.15 mmol, 5 eq.). The mixture was stirred at 70° C. for 3 h. The reaction mixture was filtered and concentrated under reduced pressure to give a residue. The residue was purified by flash silica gel chromatography (hexanes to hexanes/EtOAc=1/2, TLC: hexanes/EtOAc=2/1, R_{f}=0.3) to give (R)-7-((benzyloxy)methyl)-3-methyl-6,7-dihydropyrazolo[1,5-a]pyrazin-4(5H)-one (1.1 g, 4.05 mmol, 80.60% yield) as yellow oil: ¹H NMR (400 MHz, DMSO-d₆) δ=8.00 (br s, 1H), 7.40 (s, 1H), 7.38-7.25 (m, 5H), 4.62-4.55 (m, 1H), 4.51 (s, 2H), 3.77-3.65 (m, 3H), 3.60-3.45 (m, 1H), 2.20 (s, 3H).

### Intermediate 100: Synthesis of 2-(hydroxymethyl)-3-methyl-6,7-dihydropyrazolo[1,5-a]pyrazin-4(5H)-one

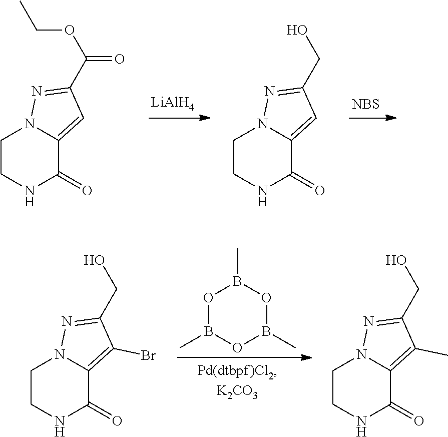

Step A: To a solution of ethyl 4-oxo-4,5,6,7-tetrahydropyrazolo[1,5-a]pyrazine-2-carboxylate (1 g, 4.78 mmol, 1 eq.) in THF (10 mL) was added LiAlH₄ (2.5 M, 3.82 mL, 2 eq.) at 0° C. under N₂ atmosphere. The mixture was stirred at 0° C. for 2 h under N₂ atmosphere. Then, H₂O (4 mL) was added into the mixture to quench the reaction, then HCl (1 N, 6 mL) was added into the mixture, the mixture was stirred at 25° C. for 10 min. The mixture was filtered and the filtrate was diluted with water (5 mL×2) and washed by EtOAc (3 mL×3). The crude product was purified by reversed-phase HPLC (Water to Water/MeCN=99/1) to yield 2-(hydroxymethyl)-6,7-dihydropyrazolo[1,5-a]pyrazin-4(5H)-one (490 mg, 2.78 mmol, 58.26% yield) as a white solid. LCMS [M+H]⁺=168.0; ¹H NMR (400 MHz, DMSO-d₆) δ=8.16 (br s, 1H), 6.61 (s, 1H), 5.12 (t, J=5.6 Hz, 1H), 4.41 (d, J=5.6 Hz, 2H), 4.26-4.18 (m, 2H), 3.63-3.51 (m, 2H).

Step B: To a solution of 2-(hydroxymethyl)-6,7-dihydropyrazolo[1,5-a]pyrazin-4(5H)-one (470 mg, 2.81 mmol, 1 eq.) in MeCN (8 mL) was added NBS (750.63 mg, 4.22 mmol, 1.5 eq.). The mixture was stirred at 25° C. for 16 h. Then, the reaction was diluted with EtOAc (3 mL×3), washed successively with water (1 mL) and brine (1 mL). The organic layer was dried over anhydrous sodium sulfate and concentrated under vacuum. The residue was purified by flash silica gel chromatography (DCM to DCM/MeOH=92/8) to yield 3-bromo-2-(hydroxymethyl)-6,7-dihydropyrazolo[1,5-a]pyrazin-4(5H)-one (120 mg, 438.92 μmol, 15.61% yield) as a white solid. LCMS [M+H]⁺=246.1; ¹H NMR (400 MHz, DMSO-d₆) δ=8.30 (br s, 1H), 5.14 (t, J=5.6 Hz, 1H), 4.40 (d, J=5.2 Hz, 2H), 4.32-4.26 (m, 2H), 3.61-3.54 (m, 2H).

Step C: A mixture of 3-bromo-2-(hydroxymethyl)-6,7-dihydropyrazolo[1,5-a]pyrazin-4(5H)-one (60 mg, 243.84 μmol, 1 eq.), trimethylboroxine (122.44 mg, 487.68 μmol, 136.35 μL, 2 eq.), K₂CO₃ (101.10 mg, 731.53 μmol, 3 eq.), and Pd (dtbpf) C12 (15.89 mg, 24.38 μmol, 0.1 eq.) in dioxane (2 mL) and H₂O (0.2 mL) was degassed and purged with N₂ three times. Then, the mixture was stirred at 85° C. for 3 h under N₂ atmosphere. The reaction mixture was filtered and concentrated in vacuum to dryness to give a crude product. The residue was purified by flash silica gel chromatography (DCM to DCM/MeOH=95/5) to yield 2-(hydroxymethyl)-3-methyl-6,7-dihydropyrazolo[1,5-a]pyrazin-4(5H)-one (35 mg, 193.17 μmol, 79.22% yield) as a yellow solid: ¹H NMR (400 MHz, DMSO-d₆) δ=8.04 (br s, 1H), 4.93 (t, J=5.6 Hz, 1H), 4.39 (d, J=5.6 Hz, 2H), 4.17 (t, J=6.0 Hz, 2H), 3.53 (t, J=6.8 Hz, 2H), 2.21 (s, 3H).

### Intermediate 101: Synthesis of 2-(2-hydroxyethyl)-3-methyl-6,7-dihydropyrazolo[1,5-a]pyrazin-4(5H)-one

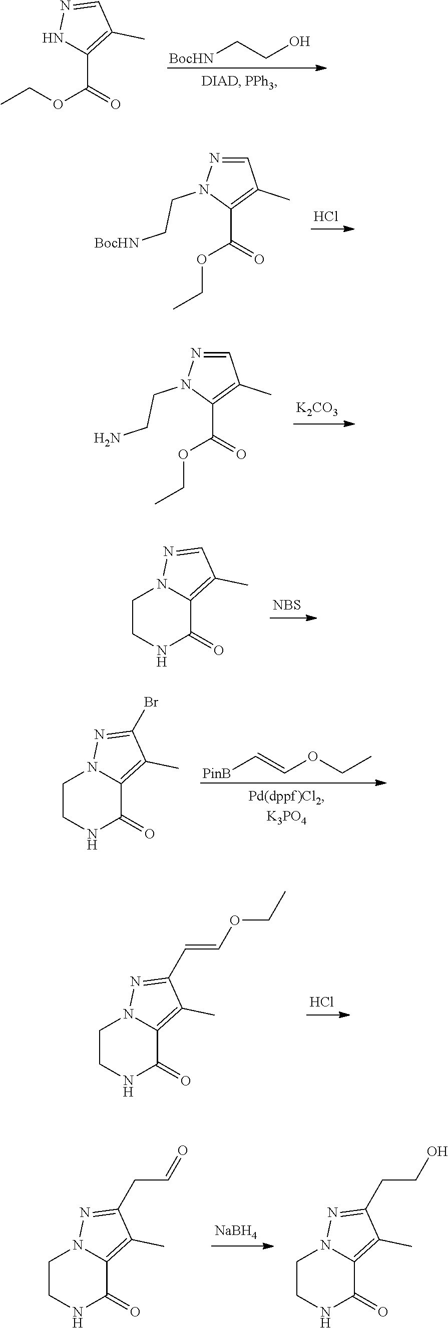

Step A: To a solution of ethyl 4-methyl-1H-pyrazole-5-carboxylate (4.5 g, 29.19 mmol, 1 eq.) and tert-butyl(2-hydroxyethyl)carbamate (6.12 g, 37.95 mmol, 5.87 mL, 1.3 eq.) in THF (90 mL) was added PPh₃ (15.31 g, 58.38 mmol, 2 eq.) and DIAD (11.80 g, 58.38 mmol, 11.32 mL, 2 eq.). The mixture was stirred at 25° C. for 12 h. Then, the reaction was diluted with H₂O (100 mL) and extracted with EtOAc (100 mL×3). The combined organic layers were washed with brine (100 mL), dried over Na₂SO₄, filtered and concentrated to yield a residue. The residue was purified by flash silica gel chromatography (hexane to hexane/EtOAc=1/4, TLC: hexane/EtOAc=2/1, R_{f}=0.8) to yield ethyl 1-(2-((tert-butoxycarbonyl)amino)ethyl)-4-methyl-1H-pyrazole-5-carboxylate (6.7 g, 20.28 mmol, 69.47% yield) as colorless oil. LCMS [M+H]⁺=298.2; ¹H NMR (400 MHz, DMSO-d₆) δ=7.40 (s, 1H), 6.81 (br t, J=5.6 Hz, 1H), 4.45 (t, J=6.0 Hz, 2H), 4.29 (q, J=7.2 Hz, 2H), 3.25 (q, J=6.0 Hz, 2H), 2.18 (s, 3H), 1.38-1.29 (m, 12H).

Step B: ethyl 1-(2-((tert-butoxycarbonyl)amino)ethyl)-4-methyl-1H-pyrazole-5-carboxylate (6.7 g, 22.53 mmol, 1 eq.) was dissolved in HCl in dioxane (70 mL). The mixture was stirred at 25° C. for 16 h. The reaction was concentrated to yield ethyl 1-(2-aminoethyl)-4-methyl-1H-pyrazole-5-carboxylate (4.4 g, 22.31 mmol, 99.01% yield) as a white solid. LCMS [M+H]⁺=198.2.

Step C: To a solution of ethyl 1-(2-aminoethyl)-4-methyl-1H-pyrazole-5-carboxylate (4.4 g, 22.31 mmol, 1 eq.) in MeOH (50 mL) was added K₂CO₃ (15.42 g, 111.54 mmol, 5 eq.). The mixture was stirred at 70° C. for 3 h. The reaction was diluted with H₂O (100 mL) and extracted with EtOAc (100 mL×3). The combined organic layers were washed with brine (100 mL), dried over Na₂SO₄, filtered, and concentrated to yield a residue. The residue was purified by flash silica gel chromatography (hexane to EtOAc) to yield 3-methyl-6,7-dihydropyrazolo[1,5-a]pyrazin-4(5H)-one (2.9 g, 17.27 mmol, 77.40% yield) as a white solid. LCMS [M+H]⁺=152.2; ¹H NMR (400 MHz, DMSO-d₆) δ=8.03 (s, 1H), 7.36 (s, 1H), 4.31-4.16 (m, 2H), 3.55 (ddd, J=2.8, 5.2, 6.8 Hz, 2H), 2.20 (s, 3H).

Step D: To a solution of 3-methyl-6,7-dihydropyrazolo[1,5-a]pyrazin-4(5H)-one (300 mg, 1.98 mmol, 1 eq.) in DMF (10 mL) was added NBS (706.45 mg, 3.97 mmol, 2 eq.). The mixture was stirred at 50° C. for 12 h. The reaction was diluted with H₂O (30 mL) and extracted with EtOAc (30 mL×3). The combined organic layers were washed with brine (30 mL), dried over Na₂SO₄, filtered and concentrated to yield a residue. The residue was purified by flash silica gel chromatography (from hexane to hexane/EtOAc=75/25) to yield 2-bromo-3-methyl-6,7-dihydropyrazolo[1,5-a]pyrazin-4(5H)-one (330 mg, 1.31 mmol, 65.77% yield) as a white solid. LCMS [M+H]⁺=230.1; ¹H NMR (400 MHz, DMSO-d₆) δ=8.23 (s, 1H), 4.28-4.16 (m, 2H), 3.61-3.50 (m, 2H), 2.15 (s, 3H).

Step E: To a solution of 2-bromo-3-methyl-6,7-dihydropyrazolo[1,5-a]pyrazin-4(5H)-one (482.12 mg, 2.43 mmol, 2 eq.) and (E)-2-(2-ethoxyvinyl)-4,4,5,5-tetramethyl-1,3,2-dioxaborolane (482.12 mg, 2.43 mmol, 2 eq) in dioxane (7 mL), H₂O (2.5 mL) was added K₃PO₄ (516.69 mg, 2.43 mmol, 2 eq.) and Pd(dppf)Cl₂·CH₂Cl₂ (99.39 mg, 121.71 μmol, 0.1 eq.) under N₂ atmosphere. The mixture was stirred at 80° C. for 4 h. The reaction was diluted with H₂O (10 mL) and extracted with EtOAc (5 mL×3). The combined organic layers were washed with brine (10 mL), dried over Na₂SO₄, filtered and concentrated to yield a residue. The residue was purified by flash silica gel chromatography (from hexane to hexane/THF=60/40, TLC: Hexane/THF=1/1, R_{f}=0.5) to yield (E)-2-(2-ethoxyvinyl)-3-methyl-6,7-dihydropyrazolo[1,5-a]pyrazin-4(5H)-one (220 mg, 994.33 μmol, 81.70% yield) as a yellow solid. LCMS [M+H]⁺=222.2; ¹H NMR (400 MHz, DMSO-d₆) δ=8.02 (s, 1H), 7.09 (d, J=12.8 Hz, 1H), 5.68 (d, J=12.8 Hz, 1H), 4.19-4.11 (m, 2H), 3.88 (q, J=7.2 Hz, 2H), 3.58-3.46 (m, 2H), 2.18 (s, 3H), 1.24 (t, J=7.2 Hz, 3H).

Step F: To a solution of (E)-2-(2-ethoxyvinyl)-3-methyl-6,7-dihydropyrazolo[1,5-a]pyrazin-4(5H)-one (200 mg, 903.93 μmol, 1 eq.) in THF (3 mL) was added HCl (6 M, 20.00 mL, 132.75 eq.). The mixture was stirred at 25° C. for 1 h. The mixture was concentrated and adjusted pH to 8 with sat. NaHCO₃. The solution was used for next step without further purification.

Step G: To the solution of 2-(3-methyl-4-oxo-4,5,6,7-tetrahydropyrazolo[1,5-a]pyrazin-2-yl) acetaldehyde (170 mg, 879.91 μmol, 1 eq.) in EtOH (3 mL) and H₂O (3 mL) was added NaBH₄ (665.78 mg, 17.60 mmol, 20 eq.) at 0° C. The mixture was stirred at 25° C. for 2 h. The reaction mixture was added sat. NH₄Cl (20 mL) in portions at 0° C. and the mixture was filtered. The filter cake was washed with EtOH (10 mL×2). The filter liquor was concentrated under reduced pressure to give a residue. The crude product was purified by reversed-phase HPLC (from H₂O to H₂O/MeCN=80/20) to yield 2-(2-hydroxyethyl)-3-methyl-6,7-dihydropyrazolo[1,5-a]pyrazin-4(5H)-one (60 mg, 276.61 μmol, 31.44% yield) as a white solid. LCMS [M+H]⁺=196.2; ¹H NMR (400 MHz, DMSO-d₆) δ=8.00 (s, 1H), 4.63 (t, J=5.2 Hz, 1H), 4.15 (t, J=6.0 Hz, 2H), 3.61-3.54 (m, 2H), 3.52 (t, J=7.2 Hz, 2H), 2.66 (t, J=7.2 Hz, 2H), 2.16 (s, 3H).

### Intermediate 102: Synthesis of (R)-7-((benzyloxy)methyl)-2,3-dimethyl-6,7-dihydropyrazolo[1,5-a]pyrazin-4(5H)-one

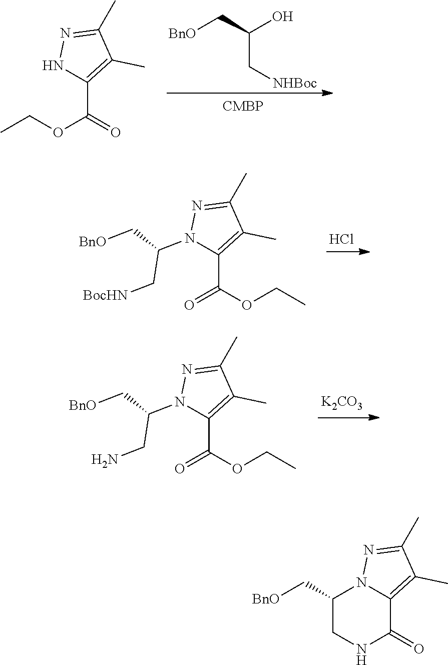

Step A: A mixture of ethyl 3,4-dimethyl-1H-pyrazole-5-carboxylate (50 mg, 297.28 μmol, 1.0 eq.), tert-butyl(S)-(3-(benzyloxy)-2-hydroxypropyl)carbamate (108.73 mg, 386.46 μmol, 1.3 eq.), (cyanomethylene)tributylphosphorane (143.50 mg, 594.56 μmol, 2 eq.) in toluene (3 mL) was degassed and purged with N₂ three times. Then, the mixture was stirred at 110° C. for 16 h under N₂ atmosphere. The reaction mixture was diluted with H₂O (20 mL) and extracted with EtOAc (50 mL×3). The combined organic layers were dried over Na₂SO₄, filtered, and concentrated under reduced pressure to give a residue. The residue was purified by flash silica gel chromatography (hexanes to hexanes/EtOAc=85/15) to give ethyl (R)-1-(1-(benzyloxy)-3-((tert-butoxycarbonyl)amino) propan-2-yl)-3,4-dimethyl-1H-pyrazole-5-carboxylate (130 mg, 96.27% yield) as yellow oil. LCMS [M+H]⁺=432.6; ¹H NMR (400 MHz, DMSO-d₆) δ=7.33-7.18 (m, 5H), 6.82-6.79 (m, 1H), 5.49-5.45 (m, 1H), 4.44-4.35 (m, 2H), 4.28-4.22 (m, 2H), 3.70-3.66 (m, 2H), 3.43-3.73 (m, 1H), 3.23-3.18 (m, 1H), 2.12 (s, 6H), 1.33 (s, 6H), 1.31-1.27 (m, 6H).

Step B: To a solution of ethyl (R)-1-(1-(benzyloxy)-3-((tert-butoxycarbonyl)amino) propan-2-yl)-3,4-dimethyl-1H-pyrazole-5-carboxylate (130 mg, 301.26 μmol, 1 eq.) in HCl/dioxane (3 mL) was stirred at 25° C. for 16 h. The reaction mixture was concentrated under reduced pressure to give ethyl (R)-1-(1-amino-3-(benzyloxy) propan-2-yl)-3,4-dimethyl-1H-pyrazole-5-carboxylate hydrochloride (150 mg, crude) as a white salt, which was used in the next step without further purification. LCMS [M+H]⁺=332.2.

Step C: To a solution of ethyl (R)-1-(1-amino-3-(benzyloxy) propan-2-yl)-3,4-dimethyl-1H-pyrazole-5-carboxylate hydrochloride (150 mg, 452.61 μmol, 1 eq, HCl) in MeOH (4 mL) was added K₂CO₃ (312.78 mg, 2.26 mmol, 5 eq.). The mixture was stirred at 70° C. for 3 h. The reaction mixture was filtered and concentrated under reduced pressure to give a residue. The residue was purified by flash silica gel chromatography (hexanes to hexanes/EtOAc=30/70) to give (R)-7-((benzyloxy)methyl)-2,3-dimethyl-6,7-dihydropyrazolo[1,5-a]pyrazin-4(5H)-one (90 mg, 58.54% yield) as a white solid. LCMS [M+H]⁺=286.2; ¹H NMR (400 MHz, CDCl₃) δ=7.37-7.29 (m, 5H), 5.59 (s, 1H), 4.59-4.52 (m, 3H), 3.84-3.73 (m, 4H), 2.25 (s, 3H), 2.21 (s, 3H).

### Intermediate 103: Synthesis of 2-(2-hydroxyethoxy)-3-methyl-6,7-dihydropyrazolo[1,5-a]pyrazin-4(5H)-one

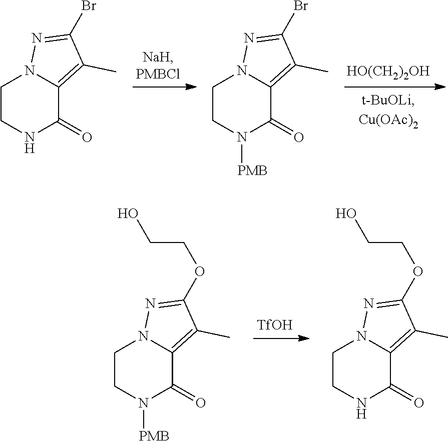

Step A: To a solution of 2-bromo-3-methyl-6,7-dihydropyrazolo[1,5-a]pyrazin-4(5H)-one (1 g, 4.35 mmol, 1 eq.) in DMF (20 mL) was added NaH (347.70 mg, 8.69 mmol, 60% purity, 2 eq.) at 0° C. under a N₂ atmosphere. Then PMB-C1 (1.36 g, 8.69 mmol, 1.18 mL, 2 eq.) was added. The mixture was stirred at 25° C. for 1 h. The reaction was quenched with H₂O (50 mL) at 0° C. and extracted with EtOAc (50 mL×3). The combined organic layers were washed with brine (50 mL), dried over Na₂SO₄, filtered and concentrated to yield a residue. The residue was purified by flash silica gel chromatography (hexane to hexane/EtOAc=60/40, TLC: hexane/EtOAc=1/1, R_{f}=0.6) to yield 2-bromo-5-(4-methoxybenzyl)-3-methyl-6,7-dihydropyrazolo[1,5-a]pyrazin-4(5H)-one (1.5 g, 3.85 mmol, 88.68% yield) as a yellow solid. LCMS [M+H]⁺=350.1; ¹H NMR (400 MHz, DMSO-d₆) δ=7.27 (s, 1H), 7.25 (s, 1H), 6.92 (s, 1H), 6.90 (s, 1H), 4.59 (s, 2H), 4.33-4.19 (m, 2H), 3.74-3.72 (m, 3H), 3.67-3.61 (m, 2H), 2.19 (s, 3H).

Step B: To a solution of 2-bromo-5-(4-methoxybenzyl)-3-methyl-6,7-dihydropyrazolo[1,5-a]pyrazin-4(5H)-one (500 mg, 1.43 mmol, 1 eq.) in ethylene glycol (11.13 g, 179.32 mmol, 10.00 mL, 125.60 eq.) was added t-BuOLi (342.89 mg, 4.28 mmol, 386.13 μL, 3 eq.) and Cu (OAc) 2 (51.86 mg, 285.54 μmol, 0.2 eq.). The mixture was stirred at 150° C. for 12 h. The reaction was diluted with H₂O (30 mL) and extracted with EtOAc (30 mL×3). The combined organic layers were washed with brine (30 mL), dried over Na₂SO₄, filtered and concentrated to yield a residue. The residue was purified by flash silica gel chromatography (hexane to hexane/EtOAc=40/60, TLC: hexane/EtOAc=1/2, R_{f}=0.3) to yield 2-(2-hydroxyethoxy)-5-(4-methoxybenzyl)-3-methyl-6,7-dihydropyrazolo[1,5-a]pyrazin-4(5H)-one (350 mg, 950.61 μmol, 66.58% yield) as yellow oil. LCMS [M+H]⁺=332.2; ¹H NMR (400 MHz, DMSO-d₆) δ=7.26 (s, 1H), 7.23 (s, 1H), 6.92 (s, 1H), 6.90 (s, 1H), 4.81 (t, J=5.6 Hz, 1H), 4.57 (s, 2H), 4.12 (t, J=5.2 Hz, 2H), 4.07 (t, J=6.0 Hz, 2H), 3.73 (s, 3H), 3.68 (q, J=5.2 Hz, 2H), 3.62-3.57 (m, 2H), 2.09 (s, 3H).

Step C: To a solution of 2-(2-hydroxyethoxy)-5-(4-methoxybenzyl)-3-methyl-6,7-dihydropyrazolo[1,5-a]pyrazin-4(5H)-one (300 mg, 905.34 μmol, 1 eq.) in toluene (5 mL) was added trifluoromethanesulfonic acid (1.36 g, 9.05 mmol, 801.14 μL, 10 eq.). The mixture was stirred at 25° C. for 1 h. The residue was diluted with ice water (10 mL) and adjusted pH to 8 with sat. NaHCO₃. The aqueous phase was purified by reversed-phase silica gel column (H₂O to H₂O/MeCN=8/92) to yield 2-(2-hydroxyethoxy)-3-methyl-6,7-dihydropyrazolo[1,5-a]pyrazin-4(5H)-one (170 mg, 724.37 μmol, 80.01% yield) as a white solid. LCMS [M+H]⁺=212.2; ¹H NMR (400 MHz, DMSO-d₆) δ=8.06 (s, 1H), 4.81 (t, J=5.6 Hz, 1H), 4.12 (t, J=5.2 Hz, 2H), 4.06-3.99 (m, 2H), 3.68 (q, J=5.2 Hz, 2H), 3.55-3.50 (m, 2H), 2.04 (s, 3H).

### Intermediate 104: (R)-7-((benzyloxy)methyl)-3-methyl-6,7-dihydro-[1,2,3]triazolo[1,5-a]pyrazin-4(5H)-one

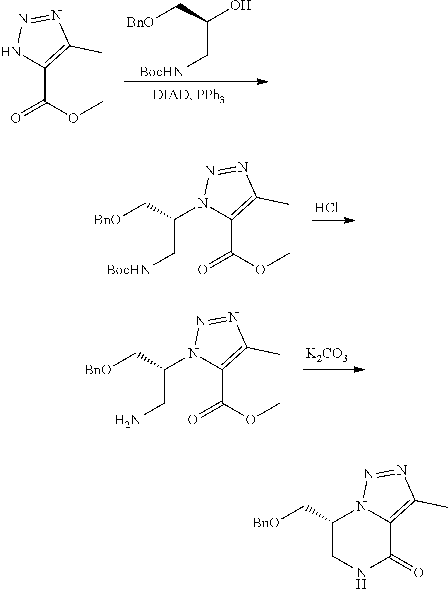

Step A: A mixture of methyl 4-methyl-1H-1,2,3-triazole-5-carboxylate (500 mg, 3.54 mmol, 1.0 eq.), tert-butyl(S)-(3-(benzyloxy)-2-hydroxypropyl)carbamate (1.30 g, 4.61 mmol, 1.3 eq.) in THF (10 mL) was added DIAD (1.43 g, 7.09 mmol, 1.37 mL, 2 eq.) and PPh₃ (1.86 g, 7.09 mmol, 2 eq.). The mixture was degassed and purged with N₂ three times. Then, the mixture was stirred at 50° C. for 2 h under N₂ atmosphere. The reaction mixture was diluted with 20 mL H₂O (20 mL) and extracted with EtOAc (30 mL×3). The combined organic layers were dried over Na₂SO₄, filtered, and concentrated under reduced pressure to give a residue. The residue was purified by prep-HPLC (column: Welch Xtimate C18 150*25 mm*5 μm; mobile phase: [H₂O (0.225% FA)-ACN]; gradient: 55%-75% ACN over 11.0 min) to give methyl (R)-1-(1-(benzyloxy)-3-((tert-butoxycarbonyl)amino) propan-2-yl)-4-methyl-1H-1,2,3-triazole-5-carboxylate (300 mg, 20.94% yield) as yellow oil. LCMS [M+H]⁺=405.1; ¹H NMR (400 MHz, DMSO-d₆) δ=7.34-7.24 (m, 3H), 7.21-7.17 (m, 2H), 7.01-6.97 (m, 1H), 5.55-5.48 (m, 1H), 4.44-4.43 (m, 2H), 3.93-3.89 (m, 2H), 3.85 (s, 3H), 3.53-3.40 (m, 2H), 2.42 (s, 3H), 1.30 (s, 9H).

Step B: To a solution of methyl (R)-1-(1-(benzyloxy)-3-((tert-butoxycarbonyl)amino) propan-2-yl)-4-methyl-1H-1,2,3-triazole-5-carboxylate (270 mg, 667.56 μmol, 1 eq.) in HCl in dioxane (5 mL) was stirred at 25° C. for 16 h. The reaction mixture was concentrated under reduced pressure to give methyl (R)-1-(1-amino-3-(benzyloxy) propan-2-yl)-4-methyl-1H-1,2,3-triazole-5-carboxylate (220 mg, crude, HCl) as a white solid, which was used in the next step without further purification. LCMS [M+H]⁺=305.2.

Step C: To a solution of methyl (R)-1-(1-amino-3-(benzyloxy) propan-2-yl)-4-methyl-1H-1,2,3-triazole-5-carboxylate (220 mg, 722.87 μmol, 1 eq, HCl) in MeOH (5 mL) was added K₂CO₃ (499.54 mg, 3.61 mmol, 5 eq.). The mixture was stirred at 60° C. for 3 h. The reaction mixture was filtered and concentrated under reduced pressure to give a residue. The residue was purified by flash silica gel chromatography (hexanes to hexanes/EtOAc=20/80) to give (R)-7-((benzyloxy)methyl)-3-methyl-6,7-dihydro-[1,2,3]triazolo[1,5-a]pyrazin-4(5H)-one (170 mg, 86.37% yield) as a white solid. LCMS [M+H]⁺=273.0; ¹H NMR (400 MHz, CDCl₃) δ=7.37-7.27 (m, 5H), 5.81 (s, 1H), 4.93-4.87 (m, 1H), 4.60-4.56 (m, 2H), 4.00-3.90 (m, 2H), 3.87-3.84 (m, 2H), 2.59 (s, 3H).

### Intermediate 105: Synthesis of methyl 8-oxo-5,6,7,8-tetrahydroimidazo[1,5-a]pyrazine-1-carboxylate

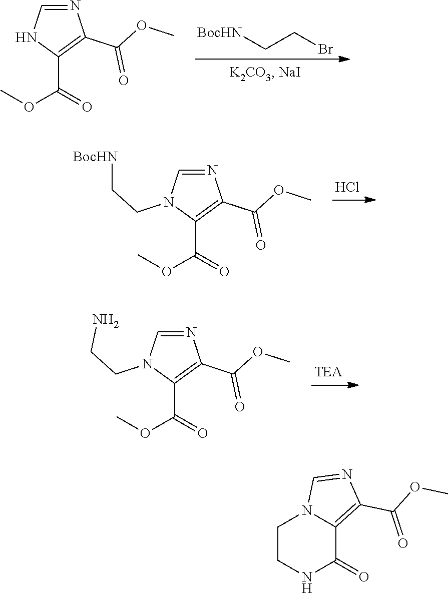

Step A: To a solution of dimethyl 1H-imidazole-4,5-dicarboxylate (4.5 g, 24.44 mmol, 1 eq.), tert-butyl(2-bromoethyl)carbamate in DMF (200 mL) was added K₂CO₃ (8.44 g, 61.09 mmol, 2.5 eq.) and NaI (3.66 g, 24.44 mmol, 1 eq.). The mixture was stirred at 90° C. for 2 h. The reaction mixture was concentrated to give a residue. The residue was diluted with H₂O (100 mL) and extracted with EtOAc (150 mL). The combined organic layers were washed with brine (50 mL), dried over sodium sulfate, filtered, and concentrated under reduced pressure to give dimethyl 1-(2-((tert-butoxycarbonyl)amino)ethyl)-1H-imidazole-4,5-dicarboxylate (6 g, 18.33 mmol, 75.01% yield) as a white solid: ¹H NMR (400 MHz, DMSO-d₆) δ=7.80 (s, 1H), 6.93 (t, J=6.0 Hz, 1H), 4.21 (t, J=5.6 Hz, 2H), 3.82-3.77 (m, 6H), 3.24 (m, 2H), 1.33 (s, 9H).

Step B: To a solution of dimethyl 1-(2-((tert-butoxycarbonyl)amino)ethyl)-1H-imidazole-4,5-dicarboxylate (3 g, 9.17 mmol, 1 eq.) in DCM (20 mL) was added HCl in dioxane (2 M, 20 mL, 4.36 eq.). The mixture was stirred at 25° C. for 1 h. The reaction mixture was concentrated to give dimethyl 1-(2-aminoethyl)-1H-imidazole-4,5-dicarboxylate (2.1 g, crude, HCl) as a white solid. LCMS [M+H]⁺=228.1.

Step C: To a solution of dimethyl 1-(2-aminoethyl)-1H-imidazole-4,5-dicarboxylate (500 mg, 2.20 mmol, 1 eq.) in MeOH (10 mL) was added TEA (2.23 g, 22.01 mmol, 3.06 mL, 10 eq.). The mixture was stirred at 25° C. for 16 h. The reaction mixture was filtered, and filter cake was concentrated under reduced pressure to give methyl 8-oxo-5,6,7,8-tetrahydroimidazo[1,5-a]pyrazine-1-carboxylate (230 mg, 1.18 mmol, 53.55% yield) as a white solid. LCMS [M+H]⁺=196.0; ¹H NMR (400 MHz, DMSO-d₆) δ=8.15 (s, 1H), 7.89 (s, 1H), 4.20 (t, J=5.6 Hz, 2H), 3.75 (s, 3H), 3.49 (t, J=6.8 Hz, 2H).

### Intermediate 106: Synthesis of 3-(2-((tert-butyldimethylsilyl)oxy)ethyl)-1-(((tert-butyldimethylsilyl)oxy)methyl)-6,7-dihydroimidazo[1,5-a]pyrazin-8(5H)-one

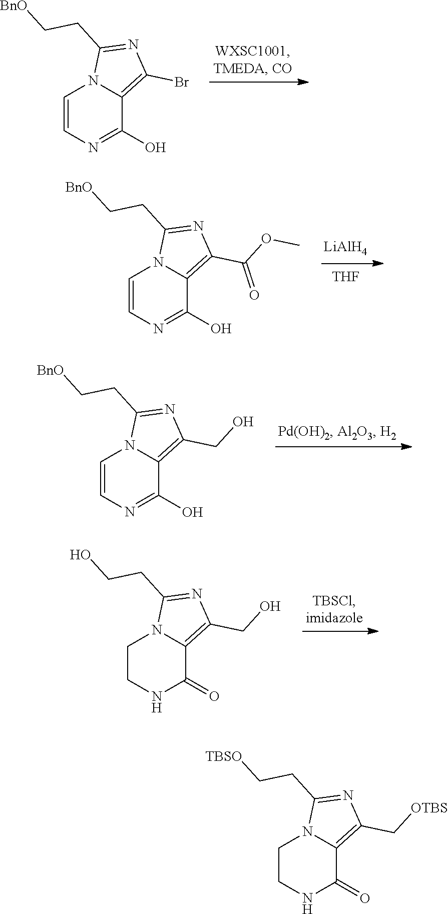

Step A: A solution of 3-(2-(benzyloxy)ethyl)-1-bromoimidazo[1,5-a]pyrazin-8-ol (1.52 g, 4.37 mmol, 1 eq.) and TMEDA (1.52 g, 13.10 mmol, 1.98 mL, 3 eq.) was prepared in MeOH (60.8 mL) and THF (30.4 mL). This solution was delivered by a plunger pump at a flow rate of 2 mL/min. Carbon monoxide (from a high-pressure cylinder) was introduced via a mass flow controller (MFC) at 20.7 sccm (1 equiv) and the system was maintained at 3 MPa. The liquid and gas streams were mixed with a T-shaped mixer and the combined solution was passed into a stainless-steel (SS) fixed-bed reactor (Outer diameter: 12.700 mm (½ inch); Internal volume: 50 mL) packed with WXPS1001 (17 g) (3% Pd/POL) and held at 160° C. Upon complete injection of the reaction solution, the solvent feed was switched to MeOH at 3 mL/min and the reactor was flushed for 30 min. The mixture was concentrated under reduced pressure to give methyl 3-(2-(benzyloxy)ethyl)-8-hydroxyimidazo[1,5-a]pyrazine-1-carboxylate (1.4 g, crude) as a brown solid, which was used in the next step without further purification. LCMS [M+H]⁺=328.0; ¹H NMR (400 MHz, DMSO-d₆) δ=11.01-10.46 (m, 1H), 7.39-7.20 (m, 6H), 6.84-6.60 (m, 1H), 4.50-4.45 (m, 2H), 3.79 (s, 3H), 3.24-3.17 (m, 2H), 2.72 (br s, 2H).

Step B: To a solution of methyl 3-(2-(benzyloxy)ethyl)-8-hydroxyimidazo[1,5-a]pyrazine-1-carboxylate (700 mg, 2.14 mmol, 1 eq.) in THF (20 mL) was added LiAlH₄ (2.5 M, 3.42 mL, 4 eq.) at 0° C. under N₂ atmosphere. The mixture was stirred at 20° C. for 2 h. H₂O (10 mL) was added into the mixture to quench the reaction. Then, aqueous NaOH (5 mL) was added into the mixture, the mixture was stirred at room temperature for 30 min, and reaction mixture was diluted with EtOAc (20 mL×3), washed successively with water (15 mL) and brine (15 mL). The organic layer was dried over anhydrous sodium sulfate and concentrated under vacuum. The residue was purified by flash silica gel chromatography (DCM to DCM/MeOH=97/3) to give 3-(2-(benzyloxy)ethyl)-1-(hydroxymethyl)imidazo[1,5-a]pyrazin-8-ol (260 mg, 847.60 μmol, 39.64% yield) as a yellow solid. LCMS [M+H]⁺=300.2; ¹H NMR (400 MHz, DMSO-d₆) δ=10.62 (br d, J=4.4 Hz, 1H), 7.36-7.16 (m, 6H), 6.59 (t, J=5.6 Hz, 1H), 5.35-5.17 (m, 1H), 4.69 (d, J=5.6 Hz, 2H), 4.48 (s, 2H), 3.88-3.67 (m, 2H), 3.22-3.00 (m, 2H).

Step C: A solution of 3-(2-(benzyloxy)ethyl)-1-(hydroxymethyl)imidazo[1,5-a]pyrazin-8-ol (640 mg, 2.14 mmol, 1 eq.) was prepared in MeOH (15 mL) and THF (15 mL). This solution was delivered by a plunger pump at a flow rate of 0.3 mL/min. Hydrogen (from a high-pressure cylinder) was introduced via a mass flow controller (MFC) at 30 sccm (2 equiv) and the system was maintained at 1.5 MPa. The liquid and gas streams were mixed with a T-shaped mixer and the combined solution was passed into a stainless-steel (SS) fixed-bed reactor (Outer diameter: 6.350 mm (¼ inch); Internal volume: 5 mL) packed with 5% Pd(OH)₂/Al₂O₃ (3.44 g, 2.14 mmol, 1 eq.) and held at 70° C. Upon complete injection of the reaction solution, the solvent feed was switched to MeOH at 10 mL/min and the reactor was flushed for 10 min. The mixture was concentrated under reduced pressure to give 3-(2-hydroxyethyl)-1-(hydroxymethyl)-6,7-dihydroimidazo[1,5-a]pyrazin-8(5H)-one (370 mg, crude) as a white solid, which was used in the next step without further purification: ¹H NMR (400 MHz, DMSO-d₆) δ=8.06 (br s, 1H), 5.51 (t, J=6.0 Hz, 1H), 4.83 (br s, 1H), 4.53 (d, J=6.0 Hz, 2H), 4.10-4.02 (m, 2H), 3.81-3.64 (m, 2H), 3.53-3.44 (m, 2H), 2.86-2.73 (m, 2H).

Step D: To a solution of 3-(2-hydroxyethyl)-1-(hydroxymethyl)-6,7-dihydroimidazo[1,5-a]pyrazin-8(5H)-one (430 mg, 2.04 mmol, 1 eq.) in DMF (6 mL) was added imidazole (831.56 mg, 12.21 mmol, 6 eq.) and tert-butyldimethylsilyl chloride (1.84 g, 12.21 mmol, 1.50 mL, 6 eq.). The mixture was stirred at 60° C. for 16 h. The reaction mixture was diluted with EtOAc (5 mL×3), washed successively with water (3 mL) and brine (3 mL). The combined organics were dried over anhydrous sodium sulfate, filtered, and concentrated under vacuum. The residue was purified by flash silica gel chromatography (hexanes to hexanes/EtOAc=73/27) to give 3-(2-((tert-butyldimethylsilyl)oxy)ethyl)-1-(((tert-butyldimethylsilyl)oxy)methyl)-6,7-dihydroimidazo[1,5-a]pyrazin-8(5H)-one (490 mg, 1.06 mmol, 52.00% yield,) as a white solid. LCMS [M+H]⁺=440.4; ¹H NMR (400 MHz, DMSO-d₆) δ=7.78 (br s, 1H), 4.78 (s, 2H), 4.10-3.98 (m, 2H), 3.89 (t, J=6.4 Hz, 2H), 3.51-3.41 (m, 2H), 2.85 (t, J=6.4 Hz, 2H), 0.85 (s, 9H), 0.81 (s, 9H), 0.06-0.01 (m, 6H), −0.03 (s, 6H).

### Intermediate 107: Synthesis of(S)-7-(2-hydroxyethyl)-3-methyl-6,7-dihydro-[1,2,3]triazolo[1,5-a]pyrazin-4(5H)-one

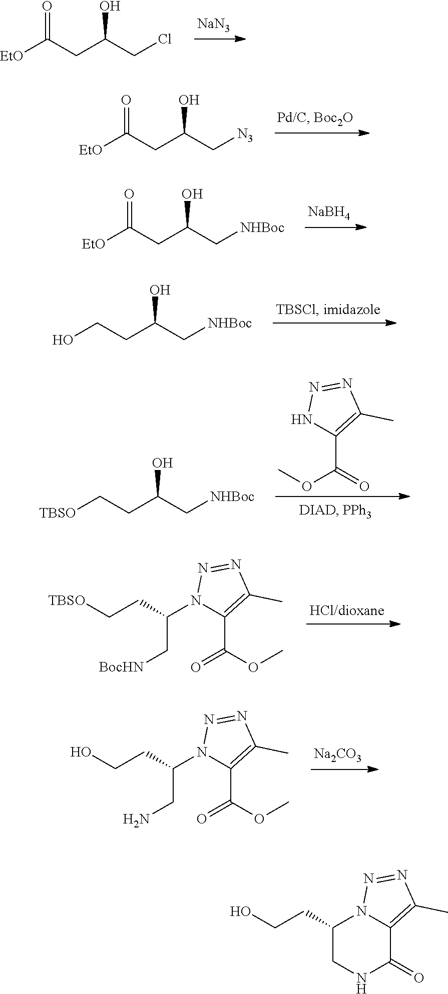

Step A: To a solution of ethyl (R)-4-chloro-3-hydroxybutanoate (5 g, 30.01 mmol, 1 eq.) in DMF (50 mL) was added NaN₃ (3.22 g, 49.53 mmol, 1.65 eq.) and the mixture was stirred at 100° C. for 3 h. Then, the mixture was filtered. The filter cake was washed with EtOAc (50 mL×3) and the combined organics were washed with brine (50 mL×3), dried over Na₂SO₄, and filtered. The filtrate was concentrated under reduced pressure to give ethyl (R)-4-azido-3-hydroxybutanoate (5 g, crude) as yellow oil. The crude product was used directly in the next step without further purification. LCMS ([M+H]⁺=174.1.

Step B: To a flask was added Pd/C (1.23 g, 1.15 mmol, 10% purity, 0.04 eq.) under argon. Then EtOAc (20 mL) was added into the mixture slowly followed by ethyl (R)-4-azido-3-hydroxybutanoate (5 g, 28.87 mmol, 1 eq.) and Boc₂O (7.56 g, 34.65 mmol, 7.96 mL, 1.2 eq.) in EtOAc (50 mL) slowly. Then the resulting mixture was purged with hydrogen several times and stirred at 25° C. for 12 h under an atmosphere of hydrogen (balloon, 15 psi).

The mixture was filtered. The filter cake was washed with EtOAc (50 mL×3). The combined organics were concentrated under reduced pressure to give a residue. The crude product was purified by flash silica gel chromatography (hexane to hexane/EtOAc=70/40) to give ethyl (R)-4-((tert-butoxycarbonyl)amino)-3-hydroxybutanoate (4.9 g, 17.83 mmol, 61.76% yield) as yellow oil. LCMS [M-Boc+H]⁺=192.1; ¹H NMR (400 MHz, CDCl₃) δ=4.99 (s, 1H), 4.25-4.08 (m, 3H), 3.50 (s, 1H), 3.44-3.27 (m, 1H), 3.21-3.03 (m, 1H), 2.51-2.46 (m, 2H), 1.45 (s, 9H), 1.28 (t, J=7.2 Hz, 3H).

Step C: To a solution of ethyl (R)-4-((tert-butoxycarbonyl)amino)-3-hydroxybutanoate (4.9 g, 19.82 mmol, 1 eq.) in EtOH (50 mL) was added NaBH₄ (4.360 g, 115.25 mmol, 5.82 eq.) in portions at 0° C. The mixture was stirred at 80° C. for 4 h under nitrogen. Then, sat. aq. NH₄Cl (100 mL) was added in portions at 0° C. and the reaction mixture was extracted with EtOAc (80 mL×3). The combined organic layers were washed with brine (100 mL×2), dried over Na₂SO₄, filtered and concentrated under reduced pressure to give tert-butyl (R)-(2,4-dihydroxybutyl)carbamate (3.69 g, crude) as yellow oil. The crude product was used directly in the next step without further purification: ¹H NMR (400 MHz, CDCl₃) δ=5.12 (s, 1H), 3.99-3.80 (m, 3H), 3.57-2.74 (m, 4H), 1.73-1.61 (m, 2H), 1.44 (s, 9H).

Step D: To a solution of tert-butyl (R)-(2,4-dihydroxybutyl)carbamate (3.68 g, 17.93 mmol, 1 eq.) and imidazole (2.44 g, 35.86 mmol, 2 eq.) in DCM (50 mL) was added TBSCl (4.05 g, 26.89 mmol, 3.31 mL, 1.5 eq.) at 0° C. and the mixture was stirred at 25° C. for 3 h. Water (80 mL) was added and the reaction was then extracted with DCM (60 mL×3). The separated organic layers were washed with brine (10 mL×2), dried over Na₂SO₄, and filtered. The filtrate was concentrated under reduced pressure to give the crude product. The crude product was purified by flash silica gel chromatography (hexane to hexane/EtOAc=70/30) to give tert-butyl (R)-(4-((tert-butyldimethylsilyl)oxy)-2-hydroxybutyl)carbamate (3.3 g, 9.81 mmol, 54.73% yield) as colorless oil: ¹H NMR (400 MHz, CDCl₃) δ=5.00 (s, 1H), 3.97-3.77 (m, 3H), 3.74 (s, 1H), 3.37-3.25 (m, 1H), 3.12-3.03 (m, 1H), 1.73-1.60 (m, 2H), 1.45 (s, 9H), 0.90 (s, 9H), 0.09 (s, 6H).

Step E: To a solution of tert-butyl (R)-(4-((tert-butyldimethylsilyl)oxy)-2-hydroxybutyl)carbamate (2 g, 6.26 mmol, 1 eq.), methyl 4-methyl-1H-1,2,3-triazole-5-carboxylate (883.40 mg, 6.26 mmol, 1 eq.) and PPh₃ (3.28 g, 12.52 mmol, 2 eq.) in THF (40 mL) was added DIAD (2.53 g, 12.52 mmol, 2.43 mL, 2 eq.) and the mixture was stirred at 50° C. for 2 h under N₂. Water (50 mL) was added and then the reaction was extracted with EtOAc (60 mL×3). The combined organic layers were washed with brine (100 mL×2), dried over Na₂SO₄, and filtered. The filtrate was concentrated under reduced pressure to give the crude product. The crude product was purified by flash silica gel chromatography (hexane to hexane/EtOAc=70/30) and reversed-phase silica gel chromatography (H₂O to H₂O/MeCN=30/70) to give methyl(S)-1-(2,2,3,3,12,12-hexamethyl-10-oxo-4,11-dioxa-9-aza-3-silatridecan-7-yl)-4-methyl-1H-1,2,3-triazole-5-carboxylate (512 mg, 1.45 mmol, 55.46% yield) as colorless oil. LCMS [M+H]⁺=443.3; ¹H NMR (400 MHz, DMSO-d₆) δ=6.94 (t, J=6.0 Hz, 1H), 5.50-5.37 (m, 1H), 3.85 (s, 3H), 3.62-3.53 (m, 1H), 3.49-3.38 (m, 1H), 3.32-3.22 (m, 2H), 2.44-2.37 (m, 3H), 2.30-2.19 (m, 1H), 2.12-1.98 (m, 1H), 1.33-1.24 (m, 9H), 0.78 (s, 9H), −0.09 (d, J=14.2 Hz, 6H).

Step F: To a solution of methyl(S)-1-(2,2,3,3,12,12-hexamethyl-10-oxo-4,11-dioxa-9-aza-3-silatridecan-7-yl)-4-methyl-1H-1,2,3-triazole-5-carboxylate (250 mg, 564.81 μmol, 1 eq.) in DCM (4 mL) was added HCl in dioxane (2 M, 10 mL, 35.41 eq.) and the mixture was stirred at 25° C. for 2 h. The mixture was concentrated under reduced pressure to give methyl (S)-1-(1-amino-4-hydroxybutan-2-yl)-4-methyl-1H-1,2,3-triazole-5-carboxylate (230 mg, crude) as yellow solid. The crude product was used directly in the next step without further purification. LCMS [M+H]⁺=229.1.

Step G: To a solution of methyl(S)-1-(1-amino-4-hydroxybutan-2-yl)-4-methyl-1H-1,2,3-triazole-5-carboxylate (230 mg, 1.01 mmol, 1 eq, crude) in MeOH (6 mL) was added Na₂CO₃ (320.41 mg, 3.02 mmol, 3 eq.) and the mixture was stirred at 25° C. for 3 h. The mixture was filtered. The filter cake was washed with EtOAc (10 mL×3), The filter liquor was concentrated under reduced pressure to give a residue. The crude product was purified by flash silica gel chromatography (DCM to DCM/MeOH=88/12, TLC: DCM/MeOH=10/1, R_{f}=0.15) to give(S)-7-(2-hydroxyethyl)-3-methyl-6,7-dihydro-[1,2,3]triazolo[1,5-a]pyrazin-4(5H)-one (90 mg, 435.77 μmol, 43.24% yield) as a white solid. LCMS [M+H]⁺=197.1; ¹H NMR (400 MHz, DMSO-d₆) δ=8.30 (s, 1H), 4.86-4.71 (m, 2H), 3.79-3.70 (m, 1H), 3.60-3.52 (m, 2H), 3.51-3.43 (m, 1H), 2.41 (s, 3H), 2.21-2.09 (m, 1H), 1.97-1.84 (m, 1H).

### Intermediate 108: Synthesis of (R)-3-(2-((tert-butyldimethylsilyl)oxy)ethyl)-5-(((tert-butyldimethylsilyl)oxy)methyl)-1-methyl-6,7-dihydroimidazo[1,5-a]pyrazin-8(5H)-one

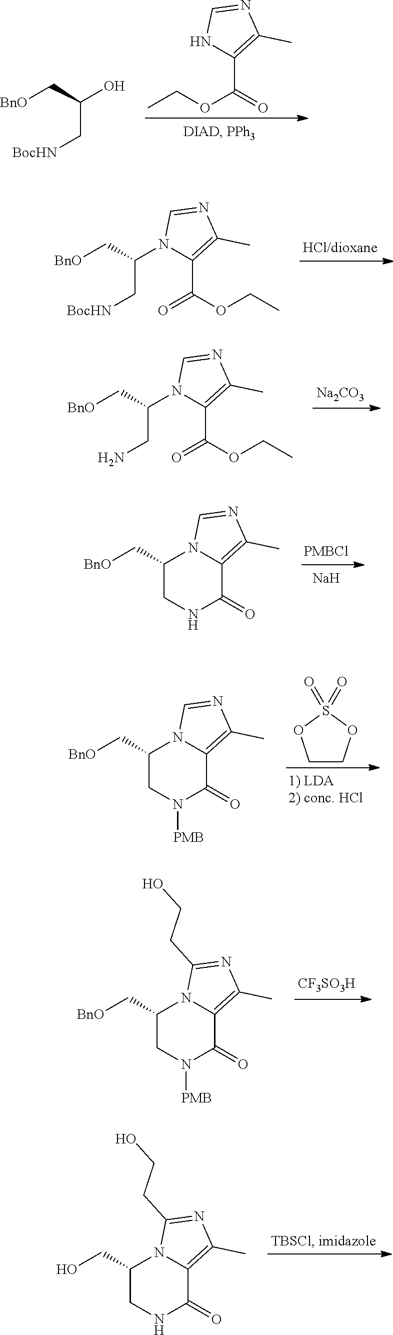

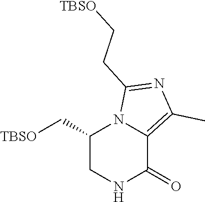 [SMILES: Cc1nc(CCO[Si](C)(C)C(C)(C)C)n2c1C(=O)NC[C@@H]2CO[Si](C)(C)C(C)(C)C]

Step A: To a solution of tert-butyl(S)-(3-(benzyloxy)-2-hydroxypropyl)carbamate (4.5 g, 12.80 mmol, 1 eq.) and ethyl 4-methyl-1H-imidazole-5-carboxylate (1.97 g, 12.80 mmol, 1.0 eq.) in THF (40 mL) was added PPh₃ (6.71 g, 25.59 mmol, 2 eq.) at 20° C. under N₂ atmosphere. Next, DIAD (5.17 g, 25.59 mmol, 4.96 mL, 2 eq.) was added dropwise at 20° C. under a N₂ atmosphere. Then, the reaction mixture was stirred at 55° C. for 60 h under N₂ atmosphere. The reaction mixture was filtered and concentrated under reduced pressure to give a residue. The residue was purified by flash silica gel chromatography (DCM to DCM/MeOH=95/5, TLC: DCM/MeOH=10/1, R_{f}=0.38) to yield ethyl (R)-1-(1-(benzyloxy)-3-((tert-butoxycarbonyl)amino) propan-2-yl)-4-methyl-1H-imidazole-5-carboxylate (4.5 g, 4.85 mmol, 37.91% yield) as a yellow solid. LCMS [M+H]⁺=418.4; ¹H NMR (400 MHz, DMSO-d₆) δ=7.82 (s, 1H), 7.33-7.26 (m, 3H), 7.21 (br d, J=7.6 Hz, 2H), 6.99 (br t, J=5.6 Hz, 1H), 5.25 (br s, 1H), 4.49-4.41 (m, 2H), 4.24-4.19 (m, 2H), 3.80-3.74 (m, 1H), 3.71-3.64 (m, 1H), 3.46-3.37 (m, 2H), 2.35 (s, 3H), 1.32-1.25 (m, 12H).

Step B: To a solution of ethyl (R)-1-(1-(benzyloxy)-3-((tert-butoxycarbonyl)amino) propan-2-yl)-4-methyl-1H-imidazole-5-carboxylate (5 g, 11.98 mmol, 1 eq.) in DCM (20 mL) was added HCl in dioxane (2 M, 59.88 mL, 10 eq.) at 0° C. The mixture was stirred at 20° C. for 12 h. The reaction mixture was diluted with water (20 mL) and extracted with EtOAc (20 mL×3). The aqueous phase was adjusted pH=7-8 with aq. NaHCO₃ at 0° C., then extracted with DCM (30 mL×3). The combined organic layers were dried over Na₂SO₄, filtered, and concentrated under reduced pressure to give ethyl (R)-1-(1-amino-3-(benzyloxy) propan-2-yl)-4-methyl-1H-imidazole-5-carboxylate (1.3 g, crude) as brown oil, which was used into the next step without further purification. LCMS [M+H]⁺=318.2; ¹H NMR (400 MHz, DMSO-d₆) δ=7.89 (s, 1H), 7.32-7.27 (m, 3H), 7.23-7.19 (m, 2H), 5.13-5.00 (m, 1H), 4.45 (s, 2H), 4.24-4.20 (m, 2H), 3.83 (dd, J=6.4, 10.4 Hz, 1H), 3.74-3.69 (m, 1H), 2.97 (d, J=6.4 Hz, 2H), 2.36 (s, 3H), 1.29-1.26 (m, 3H).

Step C: To a solution of ethyl (R)-1-(1-amino-3-(benzyloxy) propan-2-yl)-4-methyl-1H-imidazole-5-carboxylate (1.4 g, 3.09 mmol, 1 eq.) in MeOH (15 mL) was added Na₂CO₃ (981.81 mg, 9.26 mmol, 3 eq.). The mixture was stirred at 60° C. for 16 h. Then, the reaction mixture was diluted with DCM (40 mL), filtered and concentrated under reduced pressure to give a residue. The residue was purified by flash silica gel chromatography (DCM to DCM/MeOH=97/3, TLC: DCM/MeOH=10/1, R_{f}=0.56) to yield (R)-5-((benzyloxy)methyl)-1-methyl-6,7-dihydroimidazo[1,5-a]pyrazin-8(5H)-one (510 mg, 1.69 mmol, 54.79% yield) as yellow oil. LCMS [M+H]⁺=272.2; ¹H NMR (400 MHz, DMSO-d₆) δ=7.71 (s, 1H), 7.63 (br s, 1H), 7.38-7.24 (m, 5H), 4.63-4.47 (m, 3H), 3.74 (dd, J=5.6, 10.0 Hz, 1H), 3.66-3.56 (m, 2H), 3.40 (td, J=4.0, 13.2 Hz, 1H), 2.34 (s, 3H).

Step D: To a solution of (R)-5-((benzyloxy)methyl)-1-methyl-6,7-dihydroimidazo[1,5-a]pyrazin-8(5H)-one (300 mg, 1.11 mmol, 1.0 eq.) in DMF (5 mL) was added NaH (53.08 mg, 1.33 mmol, 60% purity, 1.2 eq.) at 0° C. for 0.5 h. Then, PMB-C1 (225.12 mg, 1.44 mmol, 195.08 μL, 1.3 eq.) was added and the mixture was stirred at 25° C. for 2 h. The reaction mixture was quenched by water (5 mL) slowly at 0° C. under a nitrogen atmosphere and then concentrated under reduced pressure to give the crude product. The residue was purified by flash silica gel chromatography (DCM to DCM/MeOH=97/3, TLC: DCM/MeOH=10/1, R_{f}=0.68) to yield (R)-5-((benzyloxy)methyl)-7-(4-methoxybenzyl)-1-methyl-6,7-dihydroimidazo[1,5-a]pyrazin-8(5H)-one (380 mg, 873.65 μmol, 79.01% yield) as yellow oil. LCMS [M+H]⁺=392.2; ¹H NMR (400 MHz, DMSO-d₆) δ=7.69 (s, 1H), 7.40-7.12 (m, 7H), 6.94-6.82 (m, 2H), 4.70 (d, J=14.4 Hz, 1H), 4.62-4.54 (m, 1H), 4.34 (s, 2H), 3.71 (s, 3H), 3.51 (d, J=6.4 Hz, 2H), 3.45 (dd, J=4.0, 13.2 Hz, 1H), 3.17 (d, J=5.2 Hz, 2H), 2.38 (s, 3H).

Step E: A solution of (R)-5-((benzyloxy)methyl)-7-(4-methoxybenzyl)-1-methyl-6,7-dihydroimidazo[1,5-a]pyrazin-8(5H)-one (320 mg, 817.45 μmol, 1 eq.) in THF (12.8 mL) was prepared. A separate solution of LDA (2 M, 1.14 mL, 2.8 eq.) in THF was prepared. Finally, a solution of 1,2-ethylene sulfate (202.92 mg, 1.63 mmol, 2 eq.) in THF (6.4 mL) was prepared.

The solutions of (R)-5-((benzyloxy)methyl)-7-(4-methoxybenzyl)-1-methyl-6,7-dihydroimidazo[1,5-a]pyrazin-8(5H)-one and LDA were mixed with a T-shaped mixer and at −20° C., and then passed into a perfluoroalkoxy (PFA) Coils reactor (Outer diameter: 1.590 mm ( 1/16 inch); Internal volume: 0.54 mL) at −20° C., with flow rates of 18.399 mL/min and 3.201 mL/min, respectively. The effluent from this reactor was immediately mixed with the solution of 1,2-ethylene sulfate (delivered at a flow rate of 9.363 mL/min) in a second T-shaped mixer at −20° C., and the combined stream was passed into a second perfluoroalkoxy (PFA) Coils reactor (Outer diameter: 1.590 mm ( 1/16 inch); Internal volume: 0.54 mL) at −20° C. The residence times were 0.025 min for the first reactor and 0.017 min for the second reactor.

The mixture was collected with a bottle and quenched by 12 M HCl (4 mL) at 10° C. for 12 h. Once complete, the reaction mixture was quenched by addition NaHCO₃ (30 mL) at 0° C., and then extracted with DCM (30 mL×3). The combined organic layers were dried over Na₂SO₄, filtered and concentrated under reduced pressure to give a residue. The residue was purified by flash silica gel (R)-5-((benzyloxy)methyl)-3-(2-hydroxyethyl)-7-(4-methoxybenzyl)-1-methyl-6,7-dihydroimidazo[1,5-a]pyrazin-8(5H)-one (190 mg, 392.64 μmol, 48.03% yield) as yellow oil. LCMS [M+H]⁺=436.4; ¹H NMR (400 MHz, DMSO-d₆) δ=7.37-7.25 (m, 3H), 7.22 (d, J=8.4 Hz, 2H), 7.15-7.08 (m, 2H), 6.92-6.83 (m, 2H), 4.76 (t, J=5.6 Hz, 1H), 4.71-4.59 (m, 2H), 4.32 (d, J=3.2 Hz, 1H), 4.29 (s, 1H), 4.24-4.15 (m, 1H), 3.73-3.61 (m, 6H), 3.51-3.35 (m, 3H), 2.87-2.75 (m, 1H), 2.74-2.64 (m, 1H), 2.34 (s, 3H).

Step F: To a solution of R)-5-((benzyloxy)methyl)-3-(2-hydroxyethyl)-7-(4-methoxybenzyl)-1-methyl-6,7-dihydroimidazo[1,5-a]pyrazin-8(5H)-one (180.00 mg, 413.30 μmol, 1 eq.) in toluene (5 mL) was added CF₃SO₃H (620.29 mg, 4.13 mmol, 365.74 μL, 10 eq.) at 0° C. The mixture was stirred at 20° C. for 16 h. The reaction mixture was quenched with aq. NaHCO₃ (10 mL) at 0° C., and diluted with EtOAc (10 mL) and extracted with H₂O (10 mL×2). The combined aqueous layers were purified by reversed-phase silica gel column (H₂O to H₂O/MeCN=92/8) to yield (R)-3-(2-hydroxyethyl)-5-(hydroxymethyl)-1-methyl-6,7-dihydroimidazo[1,5-a]pyrazin-8(5H)-one (70 mg, 279.70 μmol, 67.67% yield) as a colorless solid. LCMS [M+H]⁺=226.2; ¹H NMR (400 MHz, DMSO-d₆) δ=7.51 (br d, J=4.8 Hz, 1H), 5.21 (br s, 1H), 4.79 (br s, 1H), 4.45-4.31 (m, 1H), 3.80-3.62 (m, 2H), 3.58-3.41 (m, 4H), 2.93-2.70 (m, 2H), 2.29 (s, 3H).

Step G: To a solution of (R)-3-(2-hydroxyethyl)-5-(hydroxymethyl)-1-methyl-6,7-dihydroimidazo[1,5-a]pyrazin-8(5H)-one (60 mg, 186.46 μmol, 1 eq.) in DMF (2 mL) was added imidazole (126.95 mg, 1.86 mmol, 10 eq.) and TBSCl (224.83 mg, 1.49 mmol, 183.54 μL, 8 eq.) at 20° C. under a N₂ atmosphere. The mixture was stirred at 60° C. for 3 h. The reaction mixture was diluted with H₂O (10 mL) and extracted with EtOAc (10 mL×3). The combined organic layers were dried over Na₂SO₄, filtered, and concentrated under reduced pressure to give a residue. The residue was purified by flash silica gel chromatography (DCM to DCM/MeOH=97/3, TLC: DCM/MeOH=10/1, R_{f}=0.61) to yield (R)-3-(2-((tert-butyldimethylsilyl)oxy)ethyl)-5-(((tert-butyldimethylsilyl)oxy)methyl)-1-methyl-6,7-dihydroimidazo[1,5-a]pyrazin-8(5H)-one (100 mg, 176.30 μmol, 94.55% yield) as a white solid. LCMS [M+H]⁺=454.2; ¹H NMR (400 MHz, DMSO-d₆) δ=7.54 (br d, J=4.8 Hz, 1H), 4.51-4.40 (m, 1H), 3.99-3.84 (m, 2H), 3.74-3.62 (m, 2H), 3.56-3.48 (m, 1H), 3.42 (br dd, J=4.8, 13.2 Hz, 1H), 2.93-2.76 (m, 2H), 2.29 (s, 3H), 0.83 (d, J=3.2 Hz, 18H), 0.03-0.09 (m, 12H).

### Intermediate 109: Synthesis of 3-(hydroxymethyl)-6,7-dihydro-[1,2,3]triazolo[1,5-a]pyrazin-4(5H)-one

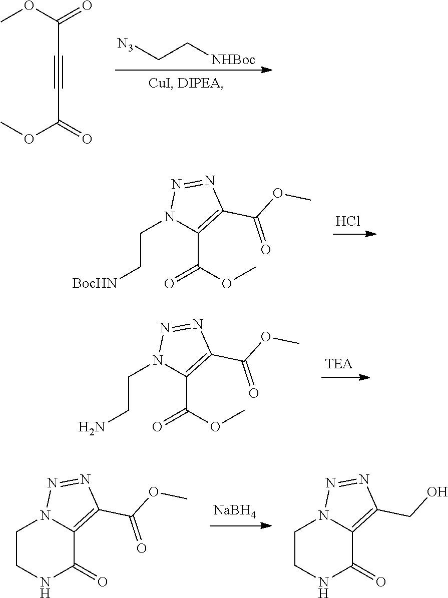

Step A: To a solution of dimethyl acetylenedicarboxylate (2 g, 14.07 mmol, 1 eq.) and tert-butyl(2-azidoethyl)carbamate (2.62 g, 14.07 mmol, 1 eq.) in THF (76 mL) and H₂O (38 mL) were added CuI (804.10 mg, 4.22 mmol, 0.3 eq.), DIPEA (5.46 g, 42.22 mmol, 7.35 mL, 3 eq.) under a N₂ atmosphere. The resulting mixture was stirred at 70° C. for 2 h. The mixture was poured into ice water (200 mL) slowly and extracted with EtOAc (200 mL×2). The separated organic layers were combined and washed with brine (200 mL×2), dried over Na₂SO₄, and filtered. The filtrate was concentrated under reduced pressure to give the crude product, which was purified by flash silica gel chromatography (hexane to hexane/EtOAc=70/30, TLC: hexane/EtOAc=1/1, R_{f}=0.73) to give dimethyl 1-(2-((tert-butoxycarbonyl)amino)ethyl)-1H-1,2,3-triazole-4,5-dicarboxylate (960 mg, 2.75 mmol, 19.53% yield) as yellow oil. LCMS [M+H]⁺=329.1; ¹H NMR (400 MHz, DMSO-d₆) δ=6.95 (t, J=6.0 Hz, 1H), 4.59 (t, J=5.6 Hz, 2H), 3.92 (s, 3H), 3.86 (s, 3H), 3.38-3.34 (m, 2H), 1.31 (s, 9H).

Step B: To a solution of dimethyl 1-(2-((tert-butoxycarbonyl)amino)ethyl)-1H-1,2,3-triazole-4,5-dicarboxylate (960 mg, 2.75 mmol, 94% purity, 1 eq.) in DCM (10 mL) was added HCl in dioxane (2 M, 27.49 mL, 20 eq.) at 5° C. The mixture was stirred at 20° C. for 2 h. The reaction mixture was concentrated to dryness to give dimethyl 1-(2-aminoethyl)-1H-1,2,3-triazole-4,5-dicarboxylate hydrochloride (740 mg, crude) as a yellow salt, which was used for the next step directly without further purification. LCMS [M+H]⁺=229.1; ¹H NMR (400 MHz, DMSO-d₆) δ=8.16-8.10 (m, 3H), 4.85 (t, J=6.0 Hz, 2H), 3.94 (s, 3H), 3.89 (s, 3H), 3.39-3.37 (m, 2H).

Step C: To a solution of dimethyl 1-(2-aminoethyl)-1H-1,2,3-triazole-4,5-dicarboxylate hydrochloride (740 mg, 2.74 mmol) in MeOH (10 mL) was added TEA (2.77 g, 27.40 mmol, 3.81 mL, 10 eq.) at 20° C. The mixture was stirred at 20° C. for 1 h under N₂ atmosphere. Then, the reaction was concentrated to dryness. The residue was purified by flash silica gel chromatography (DCM to DCM/MeOH=95/5, TLC: DCM/MeOH=10/1, Rf =0.58) to give methyl 4-oxo-4,5,6,7-tetrahydro-[1,2,3]triazolo[1,5-a]pyrazine-3-carboxylate (530 mg, 2.70 mmol, 98.60% yield) as a white solid. LCMS [M+H]⁺=197.1; ¹H NMR (400 MHz, DMSO-d₆) δ=8.62 (s, 1H), 4.69-4.60 (m, 2H), 3.85 (s, 3H), 3.70-3.61 (m, 2H).

Step D: To a solution of methyl 4-oxo-4,5,6,7-tetrahydro-[1,2,3]triazolo[1,5-a]pyrazine-3-carboxylate (520 mg, 2.65 mmol, 1 eq.) in THF (10 mL) and MeOH (2 mL) was added NaBH₄ (200.58 mg, 5.30 mmol, 33.24 μL, 2 eq.) at 0° C. under a N₂ atmosphere. The resulting mixture was stirred at 20° C. for 2 h. The reaction mixture was quenched by sat. NH₄Cl (40 mL) and concentrated under reduced pressure to dryness. The residue was then purified by reversed-phase HPLC [Column: C18, Condition: 0% (A: H₂O (neutral condition),

B: CH₃CN), flow rate: 40 mL/min] to give 3-(hydroxymethyl)-6,7-dihydro-[1,2,3]triazolo[1,5-a]pyrazin-4(5H)-one (350 mg, 1.98 mmol, 74.59% yield) as a white solid. LCMS [M+H]⁺=169.1; ¹H NMR (400 MHz, DMSO-d₆) δ=8.56 (s, 1H), 5.35-5.16 (m, 1H), 4.67 (s, 2H), 4.57 (t, J=6.0 Hz, 2H), 3.68-3.60 (m, 2H).

### Intermediate 110: Synthesis of ethyl 3-ethyl-4-oxo-4,5,6,7-tetrahydropyrazolo[1,5-a]pyrazine-2-carboxylate

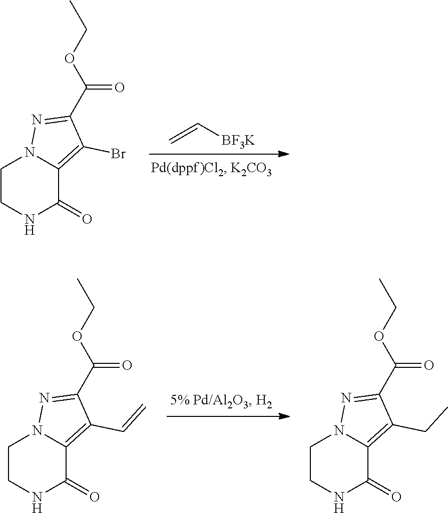

Step A: To a solution of ethyl 3-bromo-4-oxo-4,5,6,7-tetrahydropyrazolo[1,5-a]pyrazine-2-carboxylate (700 mg, 2.43 mmol, 1 eq.), potassium vinyltrifluoroborate (976.38 mg, 7.29 mmol, 3 eq.) and K₂CO₃ (1.01 g, 7.29 mmol, 3 eq.) in dioxane (20 mL) and H₂O (4 mL) was added Pd(dppf)Cl₂·CH₂Cl₂ (198.42 mg, 242.97 μmol, 0.1 eq.) and the mixture was stirred at 90° C. for 16 h under N₂. Water (40 mL) was added and then the reaction was extracted with EtOAc (50 mL×3). The combined organic layers were washed with brine (50 mL×2), dried over Na₂SO₄, and filtered. The filtrate was concentrated under reduced pressure to give the crude product. The crude product was purified by flash silica gel chromatography (DCM to DCM/MeOH=98/2, DCM/MeOH=10/1, R_{f}=0.5) to give ethyl 4-oxo-3-vinyl-4,5,6,7-tetrahydropyrazolo[1,5-a]pyrazine-2-carboxylate (470 mg, 1.70 mmol, 69.90% yield) as a yellow solid. LCMS [M+H]⁺=236.1; ¹H NMR (400 MHz, DMSO-d₆) δ=8.40 (s, 1H), 7.18 (dd, J=12.0, 18.0 Hz, 1H), 6.44 (dd, J=2.4, 18.0 Hz, 1H), 5.46 (dd, J=2.4, 12.0 Hz, 1H), 4.41-4.34 (m, 2H), 4.33-4.23 (m, 2H), 3.67-3.53 (m, 2H), 1.29 (t, J=7.2 Hz, 3H).

Step B: A solution of ethyl 4-oxo-3-vinyl-4,5,6,7-tetrahydropyrazolo[1,5-a]pyrazine-2-carboxylate (470 mg, 2.00 mmol, 1 eq.) in MeOH (11.75 mL) and THF (11.75 mL) was prepared and delivered by a plunger pump at a flow rate of 0.30 mL/min. Hydrogen gas was introduced via a mass flow controller (MFC) at 30.0 sccm (52.5 equiv) and the system was maintained at 1 MPa. The liquid and gas streams were mixed with a T-shaped mixer and the combined solution was passed into a stainless-steel (SS) fixed-bed reactor (Outer diameter: 6.350 mm (¼ inch); Internal volume: 5.00 mL) packed with 2.85 g 5% Pd/Al₂O₃ (2.85 g, 2.00 mmol, 1 eq.) and held at 50° C. Upon complete injection of the reaction solution, the solvent feed was switched to THF at 10 mL/min and the reactor was flushed for 15 min. The mixture was concentrated under reduced pressure to give ethyl 3-ethyl-4-oxo-4,5,6,7-tetrahydropyrazolo[1,5-a]pyrazine-2-carboxylate (410 mg, 1.56 mmol, 77.84% yield) as a yellow solid. The crude product was used directly in the next step without further purification. LCMS [M+H]⁺=238.2; ¹H NMR (400 MHz, DMSO-d₆) δ=8.27 (s, 1H), 4.42-4.23 (m, 4H), 3.60-3.56 (m, 2H), 2.96 (q, J=7.2 Hz, 2H), 1.28 (t, J=7.2 Hz, 3H), 1.08 (t, J=7.2 Hz, 3H).

### Intermediate 111: Synthesis of 1-(2-((tert-butyldimethylsilyl)oxy)ethyl)-3-methyl-6,7-dihydropyrazolo[1,5-a]pyrazine-2,4(1H,5H)-dione

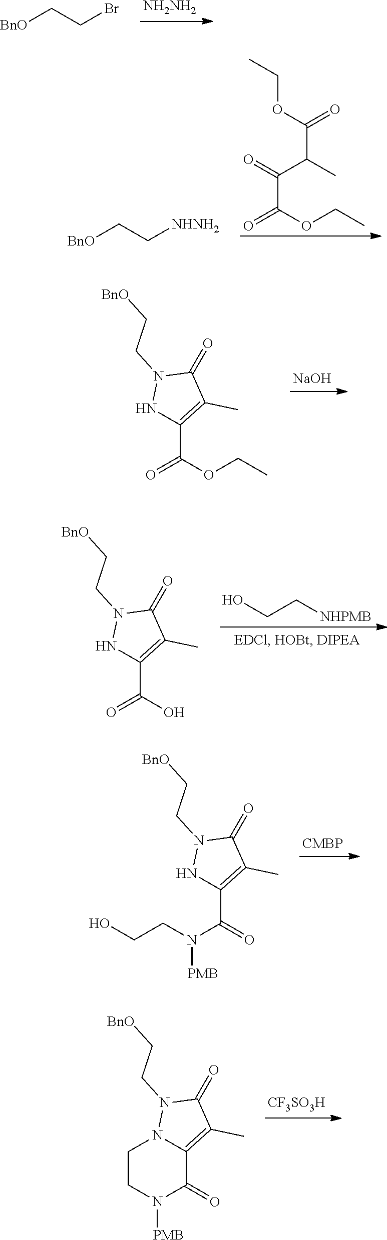

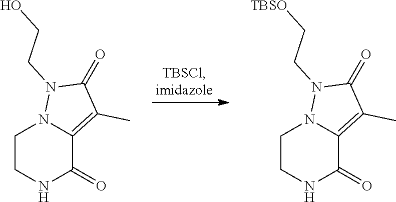 [SMILES: Cc1c2n(n(CCO)c1=O)CCNC2=O ; "TBSCl,imidazole" ; Cc1c2n(n(CCO[Si](C)(C)C(C)(C)C)c1=O)CCNC2=O]

Step A: To a solution of ((2-bromoethoxy)methyl) benzene (5 g, 23.25 mmol, 3.68 mL, 1 eq.) in EtOH (50 mL) was added hydrazine monohydrate (14.77 g, 250.79 mmol, 14.31 mL, 85% purity, 10.79 eq.) dropwise at 0° C. The mixture was stirred at 20° C. for 12 h under a N₂ atmosphere. Then, the reaction mixture was concentrated under reduced pressure. The residue was purified by reverse phase chromatography (Claricep Flash Spherical C18 Column, Spherical, 20-35 μm 100A, 80 g/column; mobile phase: [water-ACN]; gradient: 5%-20% ACN) to give (2-(benzyloxy)ethyl) hydrazine (2.9 g, 15.70 mmol, 67.55% yield) as yellow oil. LCMS [M+H]⁺=167.1; ¹H NMR (400 MHz, DMSO-d₆) δ=7.39-7.20 (m, 5H), 4.46 (s, 2H), 3.51 (t, J=5.6 Hz, 2H), 2.79 (t, J=6.0 Hz, 2H).

Step B: To a solution of (2-(benzyloxy)ethyl) hydrazine (2.9 g, 17.45 mmol, 1 eq.) in EtOH (30 mL) was added diethyl 2-methyl-3-oxosuccinate (4.23 g, 20.94 mmol, 3.87 mL, 1.2 eq.). The mixture was stirred at 80° C. for 5 h under N₂ atmosphere. The mixture was concentrated under reduced pressure. The residue was purified by flash silica gel chromatography (DCM to DCM/MeOH=90/10, TLC: DCM/MeOH=10/1, R_{f}=0.5) to give ethyl 1-(2-(benzyloxy)ethyl)-4-methyl-5-oxo-2,5-dihydro-1H-pyrazole-3-carboxylate (2 g, 5.91 mmol, 33.90% yield) as a yellow solid. LCMS [M+H]⁺=305.0; ¹H NMR (400 MHz, DMSO-d₆) δ=10.54 (s, 1H), 7.35-7.22 (m, 5H), 4.45 (s, 2H), 4.21 (q, J=7.2 Hz, 2H), 4.14 (t, J=5.6 Hz, 2H), 3.73 (t, J=6.0 Hz, 2H), 2.05 (s, 3H), 1.26 (t, J=6.4 Hz, 3H).

Step C: To a solution of ethyl 1-(2-(benzyloxy)ethyl)-4-methyl-5-oxo-2,5-dihydro-1H-pyrazole-3-carboxylate (2 g, 6.57 mmol, 1 eq.) in MeOH (16 mL), H₂O (8 mL) was added NaOH (788.59 mg, 19.71 mmol, 3 eq.). The mixture was stirred at 60° C. for 2 h. Then, the mixture was concentrated under reduced pressure to remove MeOH and the residue was adjusted pH to 5-6 with 2M HCl followed by lyophilization to give 1-(2-(benzyloxy)ethyl)-4-methyl-5-oxo-2,5-dihydro-1H-pyrazole-3-carboxylic acid (1.82 g, crude) as a yellow solid. The product was used in next step without further purification. LCMS [M+H]⁺=277.0; ¹H NMR (400 MHz, DMSO-d₆) δ=7.35-7.23 (m, 5H), 4.45 (s, 2H), 3.97 (t, J=6.0 Hz, 2H), 3.64 (t, J=6.4 Hz, 2H), 1.95 (s, 3H).

Step D: To a solution of 1-(2-(benzyloxy)ethyl)-4-methyl-5-oxo-2,5-dihydro-1H-pyrazole-3-carboxylic acid (1.2 g, 4.34 mmol, 1 eq.) in DMF (20 mL) was added 2-((4-methoxybenzyl)amino) ethanol (787.14 mg, 4.34 mmol, 1 eq.), EDCI (1.67 g, 8.69 mmol, 2 eq.), HOBt (1.17 g, 8.69 mmol, 2 eq.) and DIPEA (1.68 g, 13.03 mmol, 2.27 mL, 3 eq.). The mixture was stirred at 20° C. for 12 h under a N₂ atmosphere. The mixture was filtered and the filtrate was concentrated under vacuum. The residue was purified by flash silica gel chromatography (DCM to DCM/MeOH=90/10, TLC: DCM/MeOH=10/1, R_{f}=0.5) and then prep-HPLC (column: Welch Xtimate C18 150*25 mm*5 μm; mobile phase: [H₂O (0.225% FA)-ACN]; gradient: 0%-20% ACN over 15.0 min) to give 1-(2-(benzyloxy)ethyl)-N-(2-hydroxyethyl)-N-(4-methoxybenzyl)-4-methyl-5-oxo-2,5-dihydro-1H-pyrazole-3-carboxamide (670 mg, 1.37 mmol, 31.59% yield) as a yellow solid. LCMS [M+H]⁺=440.3; 1H NMR (400 MHz, DMSO-d₆) δ=7.33-7.20 (m, 5H), 7.17-7.14 (m, 2H), 6.90 (d, J=8.4 Hz, 1H), 6.85 (d, J=8.4 Hz, 1H), 4.78 (s, 1H), 4.62 (s, 1H), 4.44 (s, 1H), 4.35 (s, 1H), 4.06 (q, J=5.2 Hz, 2H), 3.74-3.66 (m, 5H), 3.51-3.40 (m, 3H), 3.31-3.27 (m, 1H), 1.90 (s, 3H).

Step E: To a solution of 1-(2-(benzyloxy)ethyl)-N-(2-hydroxyethyl)-N-(4-methoxybenzyl)-4-methyl-5-oxo-2,5-dihydro-1H-pyrazole-3-carboxamide (670 mg, 1.52 mmol, 1 eq.) in toluene (8 mL) was added CMBP (735.86 mg, 3.05 mmol, 2 eq.). The mixture was stirred at 100° C. for 12 h under a N₂ atmosphere. Water (10 mL) was added and the reaction was extracted with EtOAc (10 mL×3). The combined organic layers were washed with brine (20 mL), dried over Na₂SO₄, filtered and concentrated reduced pressure. The residue was purified by flash silica gel chromatography (DCM to DCM/MeOH=90/10, TLC: DCM/MeOH=20/1, R_{f}=0.5) and by prep-HPLC (column: 52-Welch Xtimate C18 150×30 mm, 5 μm; mobile phase: [H₂O (10 mM NH₄HCO₃)-ACN]; gradient: 34%-64% ACN over 11.0 min) to give 1-(2-(benzyloxy)ethyl)-5-(4-methoxybenzyl)-3-methyl-6,7-dihydropyrazolo[1,5-a]pyrazine-2,4 (1H,5H)-dione (125 mg, 266.91 μmol, 17.51% yield) as yellow oil. LCMS [M+H]⁺=422.3; ¹H NMR (400 MHz, DMSO-d₆) δ=7.27-7.18 (m, 7H), 6.93 (d, J=8.4 Hz, 2H), 4.56 (s, 2H), 4.42 (s, 2H), 3.94 (t, J=5.2 Hz, 2H), 3.75 (s, 3H), 3.55 (t, J=5.2 Hz, 2H), 3.49 (s, 4H), 2.06 (s, 3H).

Step F: To a solution of 1-(2-(benzyloxy)ethyl)-5-(4-methoxybenzyl)-3-methyl-6,7-dihydropyrazolo[1,5-a]pyrazine-2,4 (1H,5H)-dione (125 mg, 296.57 μmol, 1 eq.) in toluene (2 mL) was added dropwise CF₃SO₃H (445.09 mg, 2.97 mmol, 262.44 μL, 10 eq.) at 0° C. The mixture was stirred at 20° C. for 2 h under a N₂ atmosphere. The mixture neutralized with sat. aq. NaHCO₃ to pH=8. Then, the mixture was extracted with EtOAc (30 mL×3). The aqueous phase was concentrated under reduced pressure to give 1-(2-hydroxyethyl)-3-methyl-6,7-dihydropyrazolo[1,5-a]pyrazine-2,4 (1H,5H)-dione (62 mg, crude) as a yellow solid. The crude product was used in next step without further purification. LCMS [M+H]⁺=212.0; ¹H NMR (400 MHz, DMSO-d₆) δ=8.34 (s, 1H), 4.97-4.83 (m, 1H), 3.79 (t, J=6.0 Hz, 2H), 3.51 (s, 6H), 1.99 (s, 3H).

Step G: To a solution of 1-(2-hydroxyethyl)-3-methyl-6,7-dihydropyrazolo[1,5-a]pyrazine-2,4 (1H,5H)-dione (62 mg, 293.54 μmol, 1 eq.) in DMF (5 mL) was added imidazole (119.90 mg, 1.76 mmol, 6 eq.) and TBSCl (265.45 mg, 1.76 mmol, 216.70 μL, 6 eq.). The mixture was stirred at 50° C. for 1 h under a N₂ atmosphere. The mixture was added water (10 mL) and extracted with EtOAc (10 mL×3). The combined organic layers were washed with brine (20 mL), dried over Na₂SO₄, filtered and concentrated reduced pressure. The residue was purified with preparative TLC (DCM/MeOH=10/1, R_{f}=0.6) to give 1-(2-((tert-butyldimethylsilyl)oxy)ethyl)-3-methyl-6,7-dihydropyrazolo[1,5-a]pyrazine-2,4 (1H,5H)-dione (11 mg, 30.42 μmol, 10.36% yield) as a yellow solid. LCMS [M+H]⁺=326.1.

### Intermediate 112: Synthesis of 3-methyl-6,7-dihydro-[1,2,3]triazolo[1,5-a]pyrazin-4(5H)-one

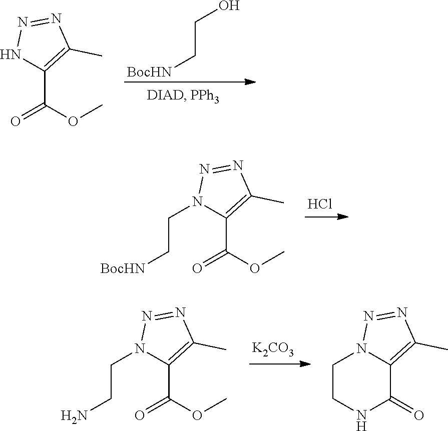

Step A: To a solution of methyl 4-methyl-1H-1,2,3-triazole-5-carboxylate (1.0 g, 7.09 mmol, 1 eq.), tert-butyl(2-hydroxyethyl)carbamate (1.14 g, 7.09 mmol, 1.10 mL, 1 eq.) and PPh₃ (3.72 g, 14.17 mmol, 2 eq.) in THF (20 mL) was added DIAD (2.87 g, 14.17 mmol, 2.75 mL, 2 eq.) at 0° C. The mixture was stirred at 50° C. for 2 h under N₂. Water (40 mL) was added then the reaction was extracted with EtOAc (50 mL×3). The combined organic layers were washed with brine (50 mL×2), dried over Na₂SO₄, and filtered. The filtrate was concentrated under vacuum to give the crude product. The crude product was purified by flash silica gel chromatography (hexane to hexane/EtOAc=75/25, hexane/EtOAc=1/1, Rf =0.4) to give methyl 1-(2-((tert-butoxycarbonyl)amino)ethyl)-4-methyl-1H-1,2,3-triazole-5-carboxylate (920 mg, 2.59 mmol, 36.53% yield) as a yellow solid. LCMS [M+H]⁺=285.2; 1H NMR (400 MHz, DMSO-d₆) δ=6.88 (t, J=5.9 Hz, 1H), 4.63 (t, J=5.7 Hz, 2H), 3.88 (s, 3H), 3.38-3.32 (m, 2H), 2.40 (s, 3H), 1.31 (s, 9H).

Step B: To a solution of methyl 1-(2-((tert-butoxycarbonyl)amino)ethyl)-4-methyl-1H-1,2,3-triazole-5-carboxylate (700 mg, 2.46 mmol, 1 eq.) in DCM (4 mL) was added HCl in dioxane (2 M, 10 mL, 8.12 eq.) and the mixture was stirred at 25° C. for 2 h. The mixture was concentrated under reduced pressure to give methyl 1-(2-aminoethyl)-4-methyl-1H-1,2,3-triazole-5-carboxylate dihydrochloride (520 mg, crude) as a white salt. The crude product was used directly in the next step without further purification. LCMS [M+H]⁺=185.1; ¹H NMR (400 MHz, DMSO-d₆) δ=8.32 (s, 2H), 4.88 (t, J=6.4 Hz, 2H), 3.90 (s, 3H), 3.37-3.24 (m, 2H), 2.45 (s, 3H).

Step C: To a solution of methyl 1-(2-aminoethyl)-4-methyl-1H-1,2,3-triazole-5-carboxylate dihydrochloride (630 mg, 3.42 mmol, 1 eq.) in MeOH (15 mL) was added K₂CO₃ (1.42 g, 10.26 mmol, 3 eq.) and the mixture was stirred at 25° C. for 3 h. The mixture was concentrated under vacuum to give a residue. The crude product was purified by flash silica gel chromatography (DCM to DCM/MeOH=95/5, DCM/MeOH=10/1, R_{f}=0.5) to give 3-methyl-6,7-dihydro-[1,2,3]triazolo[1,5-a]pyrazin-4(5H)-one (360 mg, 2.25 mmol, 65.72% yield) as a white solid. LCMS [M+H]⁺=153.2; ¹H NMR (400 MHz, DMSO-d₆) δ=8.31 (s, 1H), 4.63-4.46 (m, 2H), 3.69-3.57 (m, 2H), 2.41 (s, 3H).

### Intermediate 113: Synthesis of 3-methyl-6,7-dihydropyrazolo[1,5-a]pyrazin-4(5H)-one

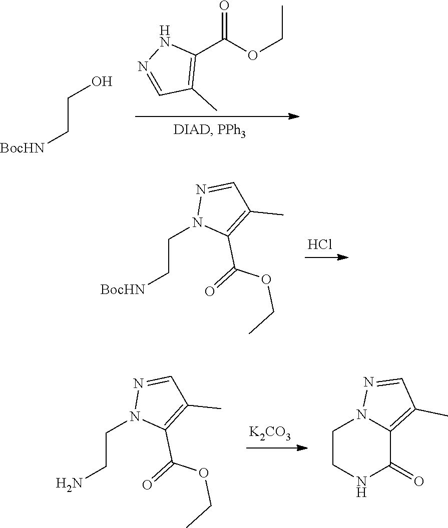

Step A: To a solution of tert-butyl(2-hydroxyethyl)carbamate (200 mg, 1.30 mmol, 1 eq.), ethyl 4-methyl-1H-pyrazole-5-carboxylate (271.86 mg, 1.69 mmol, 260.90 μL, 1.3 eq.), and PPh₃ (680.54 mg, 2.59 mmol, 2 eq.) in THF (2 mL) was added DIAD (524.65 mg, 2.59 mmol, 503.02 μL, 2 eq.). The mixture was stirred at 25° C. for 16 h. Then, the reaction mixture was diluted with EtOAc (10 mL×3), washed by water (3 mL) and brine (3 mL). The organic layer was dried over anhydrous sodium sulfate and concentrated under vacuum. The residue was purified by flash silica gel chromatography (hexanes to hexanes/EtOAc=73/27) to yield ethyl 1-(2-((tert-butoxycarbonyl)amino)ethyl)-4-methyl-1H-pyrazole-5-carboxylate (350 mg, 1.14 mmol, 88.01% yield) as colorless oil. LCMS [M+H]⁺=298.2; ¹H NMR (400 MHz, DMSO-d₆) δ=7.40 (s, 1H), 6.81 (br t, J=6.0 Hz, 1H), 4.80-4.73 (m, 2H), 4.47-4.42 (m, 2H), 4.32-4.26 (m, 2H), 2.18 (s, 3H), 1.35-1.32 (m, 9H), 1.21 (br d, J=2.8 Hz, 3H).

Step B: A mixture of ethyl 1-(2-((tert-butoxycarbonyl)amino)ethyl)-4-methyl-1H-pyrazole-5-carboxylate (350 mg, 1.18 mmol, 1 eq.) in HCl/dioxane (1 mL) was stirred at 20° C. for 2 h. The mixture was concentrated under reduced pressure to give ethyl 1-(2-aminoethyl)-4-methyl-1H-pyrazole-5-carboxylate hydrochloride (270 mg, crude) as a white salt, which was used in the next step without further purification. LCMS [M+H]⁺=198.1.

Step C: To a solution of ethyl 1-(2-aminoethyl)-4-methyl-1H-pyrazole-5-carboxylate hydrochloride (270 mg, 1.16 mmol, 1 eq.) in MeOH (3 mL) was added K₂CO₃ (638.71 mg, 4.62 mmol, 4 eq.). The mixture was stirred at 80° C. for 16 h. Then, the reaction mixture was filtered and concentrated under vacuum to dryness to give a crude product. The residue was purified by flash silica gel chromatography (DCM to DCM/MeOH=97/3) to give 3-methyl-6,7-dihydropyrazolo[1,5-a]pyrazin-4(5H)-one (130 mg, 834.19 μmol, 72.20% yield) as a white solid. LCMS [M+H]⁺=152.1; ¹H NMR (400 MHz, DMSO-d₆) δ=8.05 (br s, 1H), 7.37 (s, 1H), 4.23-4.19 (m, 2H), 3.55-3.52 (m, 2H), 2.20 (s, 3H).

### Intermediate 114: Synthesis of methyl 1-methyl-8-oxo-5,6,7,8-tetrahydroimidazo[1,5-a]pyrazine-3-carboxylate

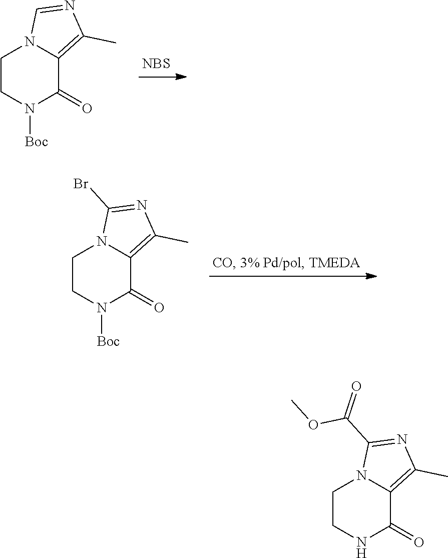

Step A: To a solution of tert-butyl 1-methyl-8-oxo-5,6-dihydroimidazo[1,5-a]pyrazine-7 (8H)-carboxylate (1 g, 3.98 mmol, 1 eq.) in MeCN (40 mL) was added NBS (1.41 g, 7.96 mmol, 2 eq.) at 20° C. Then, the reaction mixture was stirred at 60° C. for 1.5 h under a N₂ atmosphere. Next, the reaction mixture was diluted with H₂O (30 mL) and extracted with EtOAc (30 mL×3). The combined organic layers were dried over Na₂SO₄, filtered and concentrated under vacuum to give the crude product. The residue was purified by flash silica gel chromatography (hexanes to hexanes/EtOAc=68/32, TLC: hexanes/EtOAc=1/1, R_{f}=0.56) to yield tert-butyl 3-bromo-1-methyl-8-oxo-5,6-dihydroimidazo[1,5-a]pyrazine-7 (8H)-carboxylate (800 mg, 2.30 mmol, 57.84% yield) as a white solid. LCMS [M+H]⁺=330.0; ¹H NMR (400 MHz, DMSO-d₆) δ=4.21-3.96 (m, 4H), 2.37 (s, 3H), 1.48 (s, 9H).

Step B: A solution of tert-butyl 3-bromo-1-methyl-8-oxo-5,6-dihydroimidazo[1,5-a]pyrazine-7 (8H)-carboxylate (1.3 g, 3.94 mmol, 1 eq.) and TMEDA (1.37 g, 11.81 mmol, 1.78 mL, 3 eq.) in MeOH (26 mL) was prepared. A fixed bed (volume 50 mL) was completely packed with granular catalyst WXPS1001 (3% Pd/pol), 17 g. The CO back pressure regulator was adjusted to 3 Mpa, and the CO (2.0 eq.) flow rate was 20.000 sccm. The solution of tert-butyl 3-bromo-1-methyl-8-oxo-5,6-dihydroimidazo[1,5-a]pyrazine-7 (8H)-carboxylate was pumped (3 mL/min) to the flow reactor (12.700 (½″) mm, 50.000 mL, 140.0° C.). Upon completion, the reaction mixture was concentrated under reduced pressure to give the crude product. The residue was purified by flash silica gel chromatography (DCM to DCM/MeOH=98/2, TLC: DCM/MeOH=10/1, R_{f}=0.3) to yield methyl 1-methyl-8-oxo-5,6,7,8-tetrahydroimidazo[1,5-a]pyrazine-3-carboxylate (500 mg, 2.15 mmol, 54.63% yield) as a white solid. LCMS [M+H]⁺=210.1; ¹H NMR (400 MHz, DMSO-d₆) δ=8.06 (br s, 1H), 4.54-4.41 (m, 2H), 3.84 (s, 3H), 3.54-3.45 (m, 2H), 2.37 (s, 3H).

### Intermediate 115: Synthesis of 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxylic acid

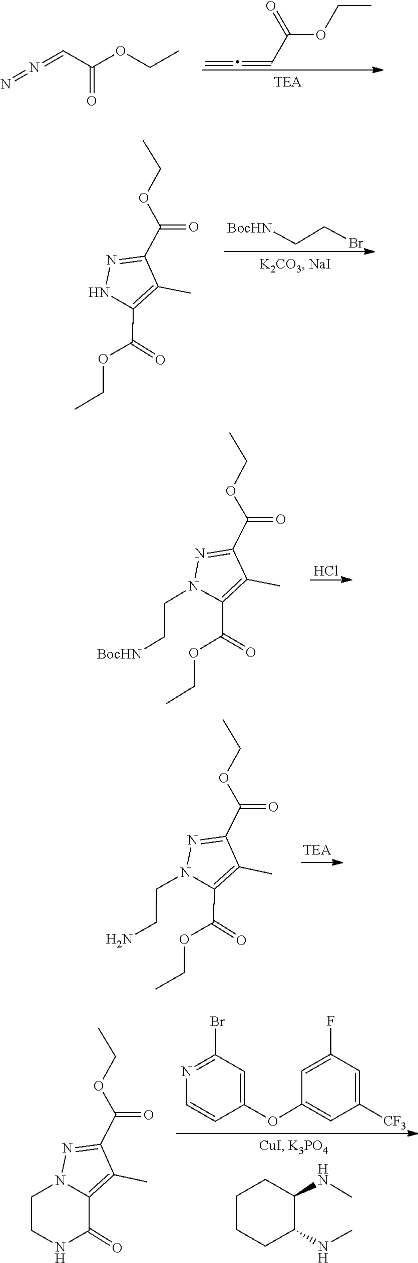

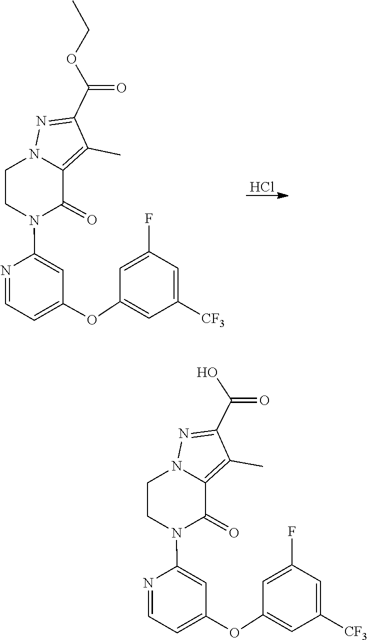 [SMILES: CCOC(=O)c1nn2c(c1C)C(=O)N(c1cc(Oc3cc(F)cc(C(F)(F)F)c3)ccn1)CC2 ; Cl ; Cc1c(C(=O)O)nn2c1C(=O)N(c1cc(Oc3cc(F)cc(C(F)(F)F)c3)ccn1)CC2]

Step A: To a solution of ethyl buta-2,3-dienoate (6 g, 53.51 mmol, 1 eq) in toluene (200 mL) was added TEA (541.47 mg, 5.35 mmol, 744.81 μL, 0.1 eq) and ethyl 2-diazoacetate (9.16 g, 80.27 mmol, 8.44 mL, 1.5 eq), the mixture was stirred at 60° C. for 12 h under a N₂ atmosphere. Then, the reaction mixture was quenched by addition 4 M HCl. The residue was diluted with H₂O (100 mL) and extracted with EtOAc (150 mL×3). The combined organic layers were dried over Na₂SO₄, filtered and concentrated under vacuum. The residue was purified by flash silica gel chromatography (hexane to hexane/EtOAc=70/30, TLC: hexane/EtOAc=1/1, R_{f}=0.6) to yield diethyl 4-methyl-1H-pyrazole-3,5-dicarboxylate (9.5 g, 41.15 mmol, 76.91% yield) as a white solid. LCMS [M+H]⁺=227.1; ¹H NMR (400 MHz, DMSO-d₆) δ=14.29 (s, 1H), 4.36-4.24 (m, 4H), 2.44 (s, 3H), 1.31 (d, J=7.2 Hz, 6H).

Step B: To a solution of diethyl 4-methyl-1H-pyrazole-3,5-dicarboxylate (4 g, 17.68 mmol, 1 eq) and tert-butyl(2-bromoethyl)carbamate (5.94 g, 26.52 mmol, 1.5 eq) in DMF (50 mL) was added K₂CO₃ (7.33 g, 53.04 mmol, 3 eq) and NaI (2.65 g, 17.68 mmol, 1 eq), the mixture was stirred at 90° C. for 1 h under a N₂ atmosphere. Then, the reaction mixture was filtrated and concentrated under vacuum. The residue was purified by flash silica gel chromatography (hexane to hexane/EtOAc=70/30, TLC: hexane/EtOAc=1/1, R_{f}=0.6) to yield diethyl 1-(2-((tert-butoxycarbonyl)amino)ethyl)-4-methyl-1H-pyrazole-3,5-dicarboxylate (6 g, 15.43 mmol, 87.27% yield) as a white solid. LCMS [M+H]⁺=370.3; ¹H NMR (400 MHz, DMSO-d₆) δ=6.88 (t, J=6.0 Hz, 1H), 4.51 (t, J=6.0 Hz, 2H), 4.38-4.19 (m, 4H), 3.32-3.26 (m, 2H), 2.42 (s, 3H), 1.39-1.18 (m, 15H).

Step C: To a mixture of diethyl 1-(2-((tert-butoxycarbonyl)amino)ethyl)-4-methyl-1H-pyrazole-3,5-dicarboxylate (6 g, 16.24 mmol, 1 eq) in HCl (10 mL, 4M in dioxane), the mixture was stirred at 20° C. for 1 h. The reaction mixture was concentrated under vacuum to yield diethyl 1-(2-aminoethyl)-4-methyl-1H-pyrazole-3,5-dicarboxylate dihydrochloride (4.9 g, crude) as a white salt. LCMS [M+H]⁺=270.2; ¹H NMR (400 MHz, DMSO-d₆) δ=8.26 (br s, 2H), 4.76 (t, J=6.8 Hz, 2H), 4.36 (q, J=6.8 Hz, 2H), 4.30 (q, J=6.8 Hz, 2H), 3.25 (br s, 2H), 2.45 (s, 3H), 1.32 (td, J=7.2, 18.0 Hz, 6H).

Step D: To a solution of diethyl 1-(2-aminoethyl)-4-methyl-1H-pyrazole-3,5-dicarboxylate dihydrochloride (6.5 g, 24.14 mmol, 1 eq) in EtOH (100 mL) was added TEA (36.64 g, 362.05 mmol, 50.39 mL, 15 eq), the mixture was stirred at 20° C. for 12 h. Then, the reaction mixture was filtrated and concentrated under vacuum to yield ethyl 3-methyl-4-oxo-4,5,6,7-tetrahydropyrazolo[1,5-a]pyrazine-2-carboxylate (4.6 g, crude) as a white solid. LCMS [M+H]⁺=224.1; ¹H NMR (400 MHz, DMSO-d₆) δ=8.27 (br s, 1H), 4.42-4.16 (m, 4H), 3.59 (br s, 2H), 2.44 (s, 3H), 1.29 (t, J=7.2 Hz, 3H).

Step E: To a solution of ethyl 3-methyl-4-oxo-4,5,6,7-tetrahydropyrazolo[1,5-a]pyrazine-2-carboxylate (1.5 g, 6.72 mmol, 1 eq) and 2-bromo-4-(3-fluoro-5-(trifluoromethyl) phenoxy)pyridine (3.16 g, 9.41 mmol, 1.4 eq) in dioxane (30 mL) was added K₃PO₄ (4.28 g, 20.16 mmol, 3 eq), CuI (255.95 mg, 1.34 mmol, 0.2 eq) and (1R,2R)—N,N′-dimethylcyclohexane-1,2-diamine (191.16 mg, 1.34 mmol, 0.2 eq). The mixture was stirred at 100° C. for 1 h under a N₂ atmosphere. The reaction mixture was diluted with H₂O (200 mL) and extracted with EtOAc (300 mL×3). The combined organic layers were dried over Na₂SO₄, filtered and concentrated under vacuum. The residue was purified by flash silica gel chromatography (hexane to hexane/EtOAc=60/40, TLC: hexane/EtOAc=1/1, R_{f}=0.7) to yield ethyl 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxylate (5.5 g, 11.15 mmol, 82.98% yield) as a white solid. LCMS [M+H]⁺=479.2; ¹H NMR: (400 MHz, DMSO-d₆) δ=8.44 (d, J=5.6 Hz, 1H), 7.65 (br d, J=8.4 Hz, 1H), 7.61 (d, J=2.0 Hz, 1H), 7.56 (td, J=2.4, 9.6 Hz, 1H), 7.50 (s, 1H), 6.98 (dd, J=2.0, 5.6 Hz, 1H), 4.60-4.43 (m, 4H), 4.29 (q, J=7.2 Hz, 2H), 2.48 (s, 3H), 1.30 (t, J=7.2 Hz, 3H).

Step F: To a mixture of ethyl 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxylate (2 g, 4.14 mmol, 1 eq) in HCl (8 M, 43.04 mL, 83.20 eq) was stirred at 80° C. for 1 h. The reaction mixture was concentrated under vacuum to yield 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxylic acid (2.2 g, crude) as a white solid. The crude product (100 mg) was purified by prep-HPLC (column: 55-Boston Prime C18 150×30 mm, 5 μm; mobile phase: [H₂O (0.05% NH₃H₂O+10 mM NH₄HCO₃)-ACN]; gradient: 28%-58% ACN over 10.0 min) to give 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxylic acid (40.39 mg, 89.69 μmol, 40.39% yield) as a white solid. LCMS [M+H]⁺=451.1; ¹H NMR (400 MHz, DMSO-d₆) δ=8.43 (d, J=5.6 Hz, 1H), 7.66 (br d, J=8.4 Hz, 1H), 7.61 (d, J=2.4 Hz, 1H), 7.57 (br d, J=9.6 Hz, 1H), 7.51 (s, 1H), 6.96 (dd, J=2.4, 6.0 Hz, 1H), 4.45 (s, 4H), 2.45 (s, 3H).

### Other Intermediates

The following intermediates were prepared according to the above procedures.

| | Structure and | |
|---|---|---|
| Intermediate | IUPAC name | 1H NMR |
| 1-1 | 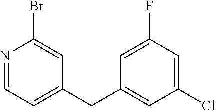 [SMILES: Fc1cc(Cl)cc(Cc2ccnc(Br)c2)c1] | ¹H NMR (400 MHz, DMSO-d₆) δ = 8.30 (d, J = 5.2 Hz, 1H), 7.63 (s, 1H), 7.42-7.06 (m, 4H), 4.00 (s, 2H) |
| | 2-bromo-4-(3-chloro-5- | |
| | fluorobenzyl)pyridine | |
| 1-2 | 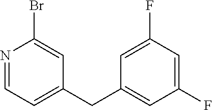 [SMILES: Fc1cc(F)cc(Cc2ccnc(Br)c2)c1] | ¹H NMR (400 MHz, CDCl₃) δ = 8.29 (d, J = 5.2 Hz, 1H), 7.29 (s, 1H), 7.05 (d, J = 5.2 Hz, 1H), 6.77-6.64 (m, 3H), 3.91 (s, 2H). |
| | 2-bromo-4-(3,5- | |
| | difluorobenzyl)pyridine | |
| 1-3 | 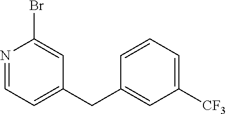 [SMILES: FC(F)(F)c1cccc(Cc2ccnc(Br)c2)c1] | ¹H NMR (400 MHz, DMSO-d₆) δ = 8.30 (d, J = 5.2 Hz, 1H), 7.72 (s, 1H), 7.6-7.44 (m, 4H), 7.35 (d, J = 4.4 Hz, 1H), 4.10 (s, 2H). |
| | 2-bromo-4-(3- | |
| | (trifluoromethyl)benzyl)pyridine | |
| 1-4 | 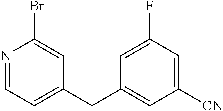 [SMILES: N#Cc1cc(F)cc(Cc2ccnc(Br)c2)c1] | ¹H NMR (400 MHz, DMSO-d₆) δ = 8.29 (d, J = 5.2 Hz, 1H), 7.74-7.69 (m, 2H), 7.65-7.58 (m, 2H), 7.40-7.34 (m, 1H), 4.05 (s, 2H). |
| | 3-((2-bromopyridin-4-yl)methyl)-5- | |
| | fluorobenzonitrile | |
| 1-5 | 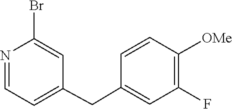 [SMILES: COc1ccc(Cc2ccnc(Br)c2)cc1F] | ¹H NMR (400 MHz, DMSO-d₆) δ = 8.26 (d, J = 5.2 Hz, 1H), 7.54 (s, 1H), 7.30 (dd, J = 1.2, 5.2 Hz, 1H), 7.18 (dd, J = 2.0, 12.4 Hz, 1H), 7.13-7.03 (m, 2H), 3.90 (s, 2H), 3.80 (s, 3H). |
| | 2-bromo-4-(3-fluoro-4- | |
| | methoxybenzyl)pyridine | |
| 1-6 | 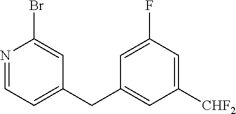 [SMILES: Fc1cc(Cc2ccnc(Br)c2)cc(C(F)F)c1] | ¹H NMR (400 MHz, DMSO-d₆) δ = 8.30 (d, J = 4.8 Hz, 1H), 7.62 (s, 1H), 7.46-7.26 (m, 4H), 7.18-6.85 (m, 1H), 4.07 (s, 2H). |
| | 2-bromo-4-(3-(difluoromethyl)-5- | |
| | fluorobenzyl)pyridine | |
| 1-7 | 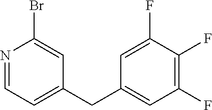 [SMILES: Fc1cc(Cc2ccnc(Br)c2)cc(F)c1F] | ¹H NMR (400 MHz, DMSO-d₆) δ = 8.30 (d, J = 5.2 Hz, 1H), 7.62 (s, 1H), 7.42-7.27 (m, 3H), 3.97 (s, 2H). |
| | 2-bromo-4-(3,4,5- | |
| | trifluorobenzyl)pyridine | |
| 1-8 | 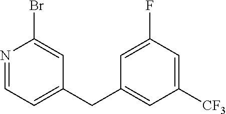 [SMILES: Fc1cc(Cc2ccnc(Br)c2)cc(C(F)(F)F)c1] | ¹H NMR (400 MHz, CDCl₃) δ = 8.32 (d, J = 5.2 Hz, 1H), 7.31 (s, 1H), 7.25 (s, 2H), 7.06 (d, J = 6.4 Hz, 2H), 4.01 (s, 2H). |
| | 2-bromo-4-(3-fluoro-5- | |
| | (trifluoromethyl)benzyl)pyridine | |
| 1-9 | 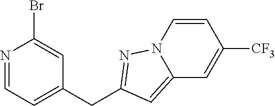 [SMILES: FC(F)(F)c1ccn2nc(Cc3ccnc(Br)c3)cc2c1] | ¹H NMR (400 MHz, CDCl₃) δ = 8.49 (d, J = 7.2 Hz, 1H), 8.29 (d, J = 4.8 Hz, 1H), 7.80 (s, 1H), 7.41 (s, 1H), 7.19 (dd, J = 0.8, 4.8 Hz, 1H), 6.91 (dd, J = 2.0, 7.6 Hz, 1H), 6.50 (s, 1H), 4.17 (s, 2H) |
| | 2-((2-bromopyridin-4-yl)methyl)-5- | |
| | (trifluoromethyl)pyrazolo[1,5-a]pyridine | |
| 1-10 | 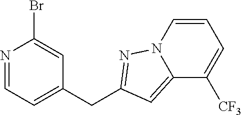 [SMILES: FC(F)(F)c1cccn2nc(Cc3ccnc(Br)c3)cc12] | ¹H NMR (400 MHz, DMSO-d₆) δ = 9.00 (d, J = 6.8 Hz, 1H), 8.22 (d, J = 5.2 Hz, 1H), 7.78 (d, J = 7.2 Hz, 1H), 7.37 (br d, J = 4.8 Hz, 1H), 7.21 (s, 1H), 7.07 (t, J = 7.2 Hz, 1H), 6.73 (s, 1H), 4.34 (s, 2H). |
| | 2-((2-bromopyridin-4-yl)methyl)-4- | |
| | (trifluoromethyl)pyrazolo[1,5- | |
| | a]pyridine | |
| 1-11 | 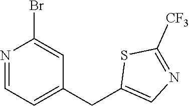 [SMILES: FC(F)(F)c1ncc(Cc2ccnc(Br)c2)s1] | ¹H NMR (400 MHz, DMSO-d₆) δ = 8.34 (d, J = 5.2 Hz, 1H), 8.04 (s, 1H), 7.66 (s, 1H), 7.40 (d, J = 4.4 Hz, 1H), 4.40 (s, 2H). |
| | 5-((2-bromopyridin-4-yl)methyl)-2- | |
| | (trifluoromethyl)thiazole | |
| 1-12 | 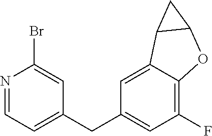 [SMILES: Fc1cc(Cc2ccnc(Br)c2)cc2c1OC1CC21] | ¹H NMR (400 MHz, DMSO-d₆) δ = 8.27 (d, J = 4.8 Hz, 1H), 7.56 (s, 1H), 7.32 (dd, J = 1.2, 4.8 Hz, 1H), 7.13 (d, J = 1.2 Hz, 1H), 7.00 (dd, J = 1.2, 11.6 Hz, 1H), 5.11-5.01 (m, 1H), 3.89 (s, 2H), 2.79-2.69 (m, 1H), 1.14-1.07 (m, 1H), 0.32-0.25 (m, 1H). |
| | 2-bromo-4-((3-fluoro-1a,6b-dihydro- | |
| | 1H-cyclopropa[b]benzofuran-5- | |
| | yl)methyl)pyridine | |
| 1-13 | 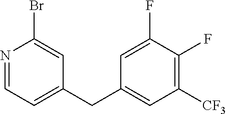 [SMILES: Fc1cc(Cc2ccnc(Br)c2)cc(C(F)(F)F)c1F] | ¹H NMR (400 MHz, CDCl₃) δ = 8.33 (d, J = 5.2 Hz, 1H), 7.30 (d, J = 0.8 Hz, 1H), 7.22-7.15 (m, 2H), 7.07-7.03 (m, 1H), 3.96 (s, 2H). |
| | 2-bromo-4-(3,4-difluoro-5- | |
| | (trifluoromethyl)benzyl)pyridine | |
| 1-14 | 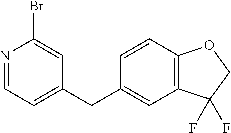 [SMILES: FC1(F)COc2ccc(Cc3ccnc(Br)c3)cc21] | ¹H NMR (400 MHz, DMSO-d₆) δ = 8.27 (d, J = 5.2 Hz, 1H), 7.59 (d, J = 8.8 Hz, 2H), 7.47 (d, J = 8.4 Hz, 1H), 7.33 (d, J = 4.8 Hz, 1H), 7.04 (d, J = 8.4 Hz, 1H), 4.78 (t, J = 16.8 Hz, 2H), 3.99 (s, 2H). |
| | 2-bromo-4-((3,3-difluoro-2,3- | |
| | dihydrobenzofuran-5- | |
| | yl)methyl)pyridine | |
| 1-15 | 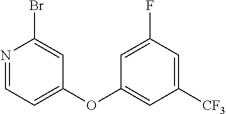 [SMILES: Fc1cc(Oc2ccnc(Br)c2)cc(C(F)(F)F)c1] | ¹H NMR (400 MHz, DMSO-d₆) δ = 8.32 (d, J = 5.6 Hz, 1H), 7.67 (br d, J = 8.8 Hz, 1H), 7.61 (td, J = 2.0, 9.6 Hz, 1H), 7.56 (s, 1H), 7.35 (d, J = 2.4 Hz, 1H), 7.11 (dd, J = 2.0, 5.6 Hz, 1H). |
| | 2-bromo-4-(3-fluoro-5- | |
| | (trifluoromethyl)phenoxy)pyridine | |
| 1-16 | 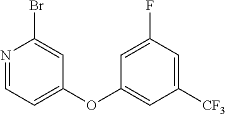 [SMILES: Fc1cc(Oc2ccnc(Br)c2)cc(C(F)(F)F)c1] | ¹H NMR (400 MHz, CDCl₃) δ = 8.29 (d, J = 5.6 Hz, 1H), 7.07 (d, J = 2.0 Hz, 1H), 6.89 (dd, J = 2.0, 5.6 Hz, 1H), 6.76 (tt, J = 2.0, 8.8 Hz, 1H), 6.69-6.62 (m, 2H). |
| | 2-bromo-4-(3,5- | |
| | difluorophenoxy)pyridine | |
| 1-17 | 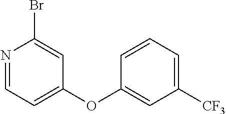 [SMILES: FC(F)(F)c1cccc(Oc2ccnc(Br)c2)c1] | ¹H NMR (400 MHz, CDCl₃) δ = 8.27 (d, J = 5.6 Hz, 1H), 7.64- 7.54 (m, 2H), 7.38 (s, 1H), 7.30 (d, J = 7.2 Hz, 1H), 7.02 (d, J = 2.0 Hz, 1H), 6.84 (dd, J = 2.0, 5.6 Hz, 1H). |
| | 2-bromo-4-(3- | |
| | (trifluoromethyl)phenoxy)pyridine | |
| 1-18 | 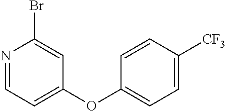 [SMILES: FC(F)(F)c1ccc(Oc2ccnc(Br)c2)cc1] | ¹H NMR (400 MHz, CDCl₃) δ = 8.29 (d, J = 5.6 Hz, 1H), 7.74 (d, J = 8.8 Hz, 2H), 7.23 (d, J = 8.4 Hz, 2H), 7.06 (d, J = 2.4 Hz, 1H), 6.91-6.86 (m, 1H). |
| | 2-bromo-4-(4- | |
| | (trifluoromethyl)phenoxy)pyridine | |
| 1-19 | 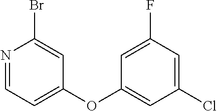 [SMILES: Fc1cc(Cl)cc(Oc2ccnc(Br)c2)c1] | ¹H NMR (400 MHz, CDCl₃) δ = 8.29 (d, J = 5.6 Hz, 1H), 7.09- 7.01 (m, 2H), 6.95-6.91 (m, 1H), 6.88 (dd, J = 2.4, 5.6 Hz, 1H), 6.76 (td, J = 2.0, 8.8 Hz, 1H). |
| | 2-bromo-4-(3-chloro-5- | |
| | fluorophenoxy)pyridine | |
| 1-20 | 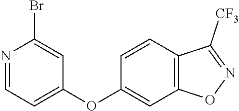 [SMILES: FC(F)(F)c1noc2cc(Oc3ccnc(Br)c3)ccc12] | ¹H NMR (400 MHz, DMSO-d₆) δ = 8.35 (d, J = 5.6 Hz, 1H), 8.09 (d, J = 8.8 Hz, 1H), 8.02 (d, J = 1.6 Hz, 1H), 7.47 (dd, J = 2.0, 8.8 Hz, 1H), 7.38 (d, J = 2.4 Hz, 1H), 7.18 (dd, J = 2.4, 5.6 Hz, 1H). |
| | 6-((2-bromopyridin-4-yl)oxy)-3- | |
| | (trifluoromethyl)benzo[d]isoxazole | |
| 1-21 | 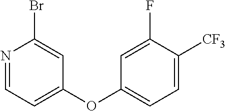 [SMILES: Fc1cc(Oc2ccnc(Br)c2)ccc1C(F)(F)F] | ¹H NMR (400 MHz, DMSO-d₆) δ = 8.35 (d, J = 5.6 Hz, 1H), 7.89 (t, J = 8.8 Hz, 1H), 7.55 (dd, J = 1.6, 11.6 Hz, 1H), 7.43 (d, J = 2.0 Hz, 1H), 7.25 (d, J = 8.8 Hz, 1H), 7.19 (dd, J = 2.0, 5.6 Hz, 1H). |
| | 2-bromo-4-(3-fluoro-4- | |
| | (trifluoromethyl)phenoxy)pyridine | |
| 1-22 | 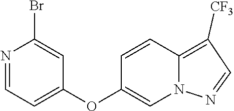 [SMILES: FC(F)(F)c1cnn2cc(Oc3ccnc(Br)c3)ccc12] | ¹H NMR (400 MHz, DMSO-d₆) δ = 9.19 (d, J = 1.2 Hz, 1 H), 8.55 (s, 1 H), 8.30 (d, J = 6.0 Hz, 1 H), 7.86-7.94 (m, 1 H), 7.56 (dd, J = 9.6, 2.0 Hz, 1 H), 7.40 (d, J = 2.4 Hz, 1 H), 7.14- 7.22 (m, 1 H). |
| | 6-((2-bromopyridin-4-yl)oxy)-3- | |
| | (trifluoromethyl)pyrazolo[1,5- | |
| | a]pyridine | |
| 1-23 | 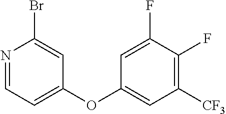 [SMILES: Fc1cc(Oc2ccnc(Br)c2)cc(C(F)(F)F)c1F] | ¹H NMR (400 MHz, DMSO-d₆) δ = 8.31 (d, J = 5.6 Hz, 1H), 8.02-7.91 (m, 1H), 7.66-7.59 (m, 1H), 7.34 (d, J = 2.4 Hz, 1H), 7.13 (dd, J = 2.0, 5.6 Hz, 1H). |
| | 2-bromo-4-(3,4-difluoro-5- | |
| | (trifluoromethyl)phenoxy)pyridine | |
| 1-24 | 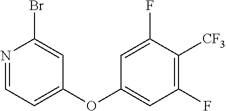 [SMILES: Fc1cc(Oc2ccnc(Br)c2)cc(F)c1C(F)(F)F] | ¹H NMR (400 MHz, CDCl₃) δ = 8.38-8.35 (m, 1H), 7.15 (d, J = 2.8 Hz, 1H), 6.97-6.93 (m, 1H), 6.74 (d, J = 9.6 Hz, 2H). |
| | 2-bromo-4-(3,5-difluoro-4- | |
| | (trifluoromethyl)phenoxy)pyridine | |
| 1-25 | 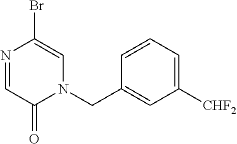 [SMILES: O=c1cnc(Br)cn1Cc1cccc(C(F)F)c1] | ¹H NMR (400 MHz, DMSO-d₆) δ = 8.26 (s, 1H), 7.96-7.91 (m, 1H), 7.46-7.37 (m, 3H), 7.19-6.88 (m, 1H), 5.10 (s, 2H). |
| | 5-bromo-1-(3-(difluoromethyl)-5- | |
| | fluorobenzyl)pyrazin-2(1H)-one | |
| 1-26 | 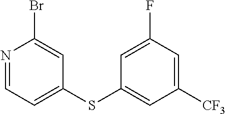 [SMILES: Fc1cc(Sc2ccnc(Br)c2)cc(C(F)(F)F)c1] | ¹H NMR (400 MHz, DMSO-d₆) δ = 8.24 (d, J = 5.6 Hz, 1H), 7.91 (d, J = 8.8 Hz, 2H), 7.85 (s, 1H), 7.46 (d, J = 1.6 Hz, 1H), 7.18 (dd, J = 2.0, 5.6 Hz, 1H). |
| | 2-bromo-4-((3-fluoro-5- | |
| | (trifluoromethyl)phenyl)thio)pyridine | |
| 1-27 | 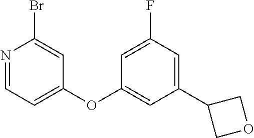 [SMILES: Fc1cc(Oc2ccnc(Br)c2)cc(C2COC2)c1] | ¹H NMR (400 MHz, DMSO-d₆) δ = 8.29 (d, J = 5.6 Hz, 1H), 7.27 (d, J = 9.6 Hz, 1H), 7.24 (d, J = 2.4 Hz, 1H), 7.15-7.09 (m, 2H), 7.04 (dd, J = 2.4, 5.4 Hz, 1H), 4.90 (dd, J = 6.0, 8.4 Hz, 2H), 4.62 (t, J = 6.4 Hz, 2H), 4.31 (t, J = 7.6 Hz, 1H). |
| | 2-bromo-4-(3-fluoro-5-(oxetan-3- | |
| | yl)phenoxy)pyridine | |
| 1-28 | 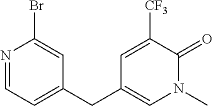 [SMILES: Cn1cc(Cc2ccnc(Br)c2)cc(C(F)(F)F)c1=O] | [M + H]⁺ = 346.9. |
| | 5-((2-bromopyridin-4-yl)methyl)-1- | |
| | methyl-3-(trifluoromethyl)pyridin- | |
| | 2(1H)-one | |
| 1-29 | 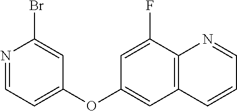 [SMILES: Fc1cc(Oc2ccnc(Br)c2)cc2cccnc12] | ¹H NMR (400 MHz, DMSO-d₆) δ = 8.97 (dd, J = 1.6, 4.0 Hz, 1H), 8.45 (d, J = 8.4 Hz, 1H), 8.33 (d, J = 5.6 Hz, 1H), 7.73- 7.63 (m, 3H), 7.38 (d, J = 2.4 Hz, 1H), 7.16 (dd, J = 2.4, 6.0 Hz, 1H) |
| | 6-((2-bromopyridin-4-yl)oxy)-8- | |
| | fluoroquinoline | |
| 1-30 | 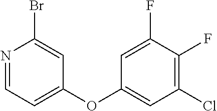 [SMILES: Fc1cc(Oc2ccnc(Br)c2)cc(Cl)c1F] | ¹H NMR (400 MHz, DMSO-d₆) δ = 8.30 (d, J = 6.0 Hz, 1H), 7.63-7.55 (m, 1H), 7.51 (td, J = 2.4, 5.2 Hz, 1H), 7.30 (d, J = 2.4 Hz, 1H), 7.10 (dd, J = 1.6, 5.6 Hz, 1H). |
| | 2-bromo-4-(3-chloro-4,5- | |
| | difluorophenoxy)pyridine | |
| 1-31 | 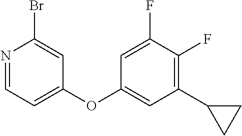 [SMILES: Fc1cc(Oc2ccnc(Br)c2)cc(C2CC2)c1F] | ¹H NMR (400 MHz, DMSO-d₆) δ = 8.27 (d, J = 6.0 Hz, 1H), 7.33-7.24 (m, 1H), 7.19 (d, J = 2.4 Hz, 1H), 7.02-6.97 (m, 1H), 6.78-6.73 (m, 1H), 2.17-2.07 (m, 1H), 1.09-0.99 (m, 2H), 0.85-0.78 (m, 2H). |
| | 2-bromo-4-(3-cyclopropyl-4,5- | |
| | difluorophenoxy)pyridine | |
| 1-32 | 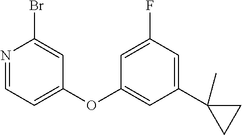 [SMILES: CC1(c2cc(F)cc(Oc3ccnc(Br)c3)c2)CC1] | ¹H NMR (400 MHz, CDCl₃) δ = 6.79 (td, J = 2.0, 10.0 Hz, 1H), 6.73 (t, J = 1.6 Hz, 1H), 6.48 (td, J = 2.4, 10.0 Hz, 1H), 5.36 (s, 1H), 5.11 (d, J = 1.2 Hz, 1H), 2.11 (s, 3H), 1.01- 0.99 (m, 9H), 0.25-0.20 (m, 6H). |
| | 2-bromo-4-(3-fluoro-5-(1- | |
| | methylcyclopropyl)phenoxy)pyridine | |
| 1-33 | 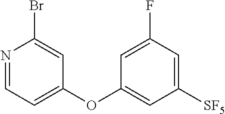 | ¹H NMR (400 MHz, CDCl₃) δ = 8.25 (d, J = 5.6 Hz, 1H), 7.36 (td, J = 2.0, 8.4 Hz, 1H), 7.27 (d, J = 1.6 Hz, 1H), 7.03 (d, J = 2.4 Hz, 1H), 6.97-6.92 (m, 1H), 6.82 (dd, J = 2.0, 5.6 Hz, 1H). ¹⁹F NMR (376 MHz, CDCl₃) δ = 63.048, −105.579. |
| | 2-bromo-4-(3-fluoro-5-(pentafluoro- | |
| | λ⁶-sulfaneyl)phenoxy)pyridine | |
| 1-34 | 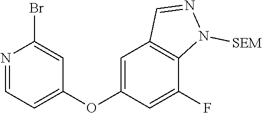 [SMILES: C[Si](C)(C)CCOCn1ncc2cc(Oc3ccnc(Br)c3)cc(F)c21] | ¹H NMR (400 MHz, DMSO-d₆) δ = 8.37-8.12 (m, 2H), 7.55 (d, J = 2.0 Hz, 1H), 7.38 (dd, J = 2.0, 12.0 Hz, 1H), 7.12 (d, J = 2.0 Hz, 1H), 7.02 (dd, J = 2.4, 6.0 Hz, 1H), 5.78 (s, 2H), 3.55 (t, J = 8.0 Hz, 2H), 0.80 (t, J = 7.6 Hz, 2H), −0.07-−0.18 (m, 9H). |
| | 5-((2-bromopyridin-4-yl)oxy)-7- | |
| | fluoro-1-((2- | |
| | (trimethylsilyl)ethoxy)methyl)-1H- | |
| | indazole | |
| 1-35 | 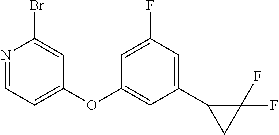 [SMILES: Fc1cc(Oc2ccnc(Br)c2)cc(C2CC2(F)F)c1] | ¹H NMR (400 MHz, DMSO-d₆) δ = 8.30 (d, J = 5.6 Hz, 1H), 7.23 (d, J = 2.4 Hz, 1H), 7.18- 7.13 (m, 2H), 7.07 (s, 1H), 7.03 (dd, J = 2.4, 5.6 Hz, 1H), 3.10 (dt, J = 8.4, 12.4 Hz, 1H), 2.20- 2.00 (m, 2H). |
| | 2-bromo-4-(3-(2,2- | |
| | difluorocyclopropyl)-5- | |
| | fluorophenoxy)pyridine | |
| 1-36 | 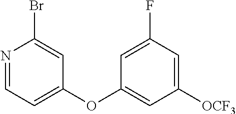 [SMILES: Fc1cc(Oc2ccnc(Br)c2)cc(OC(F)(F)F)c1] | ¹H NMR (400 MHz, CDCl₃) δ = 8.23 (d, J = 5.6 Hz, 1H), 7.01 (d, J = 2.0 Hz, 1H), 6.87-6.79 (m, 2H), 6.75-6.68 (m, 2H). |
| | 2-bromo-4-(3-fluoro-5- | |
| | (trifluoromethoxy)phenoxy)pyridine | |
| 1-37 | 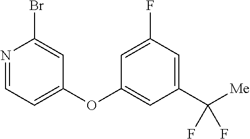 [SMILES: CC(F)(F)c1cc(F)cc(Oc2ccnc(Br)c2)c1] | ¹H NMR (400 MHz, CDCl₃) δ = 8.21 (d, J = 2.4 Hz, 1H), 7.08 (d, J = 8.4 Hz, 1H), 6.98 (d, J = 2.0 Hz, 2H), 6.82-6.79 (m, 2H), 1.85 (t, J = 18.0 Hz, 3H). |
| | 2-bromo-4-(3-(1,1-difluoroethyl)-5- | |
| | fluorophenoxy)pyridine | |
| 1-38 | 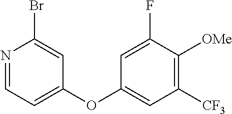 [SMILES: COc1c(F)cc(Oc2ccnc(Br)c2)cc1C(F)(F)F] | ¹H NMR (400 MHz, DMSO-d₆) δ = 8.29 (d, J = 5.6 Hz, 1H), 7.75 (dd, J = 2.8, 12.0 Hz, 1H), 7.51-7.37 (m, 1H), 7.30 (d, J = 2.4 Hz, 1H), 7.08 (dd, J = 2.4, 5.6 Hz, 1H), 3.97 (d, J = 1.6 Hz, 3H). |
| | 2-bromo-4-(3-fluoro-4-methoxy-5- | |
| | (trifluoromethyl)phenoxy)pyridine | |
| 1-39 | 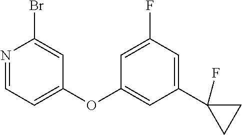 [SMILES: Fc1cc(Oc2ccnc(Br)c2)cc(C2(F)CC2)c1] | ¹H NMR (400 MHz, CDCl₃) δ = 8.26 (d, J = 6.0 Hz, 1H), 7.03 (d, J = 2.4 Hz, 1H), 6.89-6.81 (m, 2H), 6.78 (s, 1H), 6.75-6.71 (m, 1H), 1.64-1.61 (m, 1H), 1.55 (br s, 1H), 1.15-1.06 (m, 2H). |
| | 2-bromo-4-(3-fluoro-5-(1- | |
| | fluorocyclopropyl)phenoxy)pyridine | |
| 1-40 | 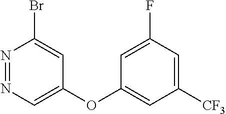 [SMILES: Fc1cc(Oc2cnnc(Br)c2)cc(C(F)(F)F)c1] | ¹H NMR (400 MHz, DMSO-d₆) δ = 9.25 (d, J = 2.8 Hz, 1H), 7.74-7.69 (m, 2H), 7.69-7.66 (m, 2H). |
| | 3-bromo-5-(3-fluoro-5- | |
| | (trifluoromethyl)phenoxy)pyridazine | |
| 1-41 | 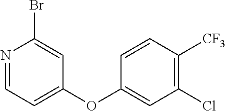 [SMILES: FC(F)(F)c1ccc(Oc2ccnc(Br)c2)cc1Cl] | ¹H NMR (400 MHz, CDCl₃) δ = 8.31 (d, J = 5.6 Hz, 1H), 7.77 (d, J = 8.8 Hz, 1H), 7.26 (s, 1H), 7.11-7.06 (m, 2H), 6.90 (dd, J = 2.4, 6.0 Hz, 1H). |
| | 2-bromo-4-(3-chloro-4- | |
| | (trifluoromethyl)phenoxy)pyridine | |
| 1-42 | 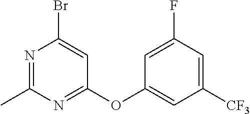 [SMILES: Cc1nc(Br)cc(Oc2cc(F)cc(C(F)(F)F)c2)n1] | ¹H NMR (400 MHz, DMSO-d₆) δ = 7.69-7.61 (m, 3H), 7.47 (s, 1H), 2.45 (s, 3H). |
| | 4-bromo-6-(3-fluoro-5- | |
| | (trifluoromethyl)phenoxy)-2- | |
| | methylpyrimidine | |
| 1-43 | 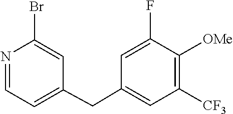 [SMILES: COc1c(F)cc(Cc2ccnc(Br)c2)cc1C(F)(F)F] | ¹H NMR (400 MHz, DMSO-d₆) δ = 8.30 (d, J = 5.2 Hz, 1H), 7.70-7.60 (m, 2H), 7.50 (s, 1H), 7.40-7.30 (m, 1H), 4.03-4.00 (m, 2H), 3.92 (d, J = 1.6 Hz, 3H). |
| | 2-bromo-4-(3-fluoro-4-methoxy-5- | |
| | (trifluoromethyl)benzyl)pyridine | |
| 1-44 | 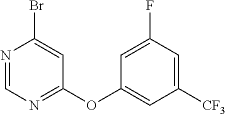 [SMILES: Fc1cc(Oc2cc(Br)ncn2)cc(C(F)(F)F)c1] | ¹H NMR (400 MHz, CDCl₃) δ = 8.56 (s, 1H), 7.32-7.27 (m, 2H), 7.24 (s, 1H), 7.14 (td, J = 2.1, 8.8 Hz, 1H). |
| | 4-bromo-6-(3-fluoro-5- | |
| | (trifluoromethyl)phenoxy)pyrimidine | |
| 1-45 | 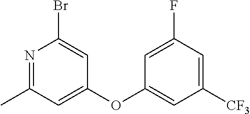 [SMILES: Cc1cc(Oc2cc(F)cc(C(F)(F)F)c2)cc(Br)n1] | ¹H NMR (400 MHz, CDCl₃) δ = 7.25 (s, 1H), 7.17 (s, 1H), 7.04-6.97 (m, 1H), 6.89 (d, J = 2.0 Hz, 1H), 6.71 (d, J = 2.0 Hz, 1H), 2.53 (s, 3H). |
| | 2-bromo-4-(3-fluoro-5- | |
| | (trifluoromethyl)phenoxy)-6- | |
| | methylpyridine | |
| 1-46 | 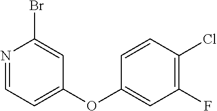 [SMILES: Fc1cc(Oc2ccnc(Br)c2)ccc1Cl] | ¹H NMR (400 MHz, DMSO-d₆) δ = 8.30 (d, J = 5.6 Hz, 1H), 7.71 (t, J = 8.8 Hz, 1H), 7.49 (dd, J = 2.8, 10.0 Hz, 1H), 7.28 (d, J = 2.0 Hz, 1H), 7.18-7.11 (m, 1H), 7.08 (dd, J = 2.4, 5.6 Hz, 1H). |
| | 2-bromo-4-(4-chloro-3- | |
| | fluorophenoxy)pyridine | |
| 1-47 | 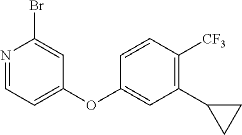 [SMILES: FC(F)(F)c1ccc(Oc2ccnc(Br)c2)cc1C1CC1] | ¹H NMR (400 MHz, DMSO-d₆) δ = 8.27 (d, J = 5.6 Hz, 1H), 7.70 (d, J = 8.8 Hz, 1H), 7.03 (d, J = 2.0 Hz, 1H), 6.99-6.93 (m, 1H), 6.86-6.83 (m, 1H), 6.75 (d, J = 2.0 Hz, 1H), 2.31- 2.21 (m, 1H), 1.17-1.07 (m, 2H), 0.80-0.74 (m, 2H). |
| | 2-bromo-4-(3-cyclopropyl-4- | |
| | (trifluoromethyl)phenoxy)pyridine | |
| 1-48 | 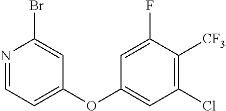 [SMILES: Fc1cc(Oc2ccnc(Br)c2)cc(Cl)c1C(F)(F)F] | ¹H NMR (400 MHz, DMSO-d₆) δ = 8.43-8.30 (m, 1H), 7.63- 7.45 (m, 3H), 7.32-7.21 (m, 1H). |
| | 2-bromo-4-(3-chloro-5-fluoro-4- | |
| | (trifluoromethyl)phenoxy)pyridine | |
| 1-49 | 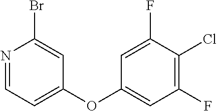 [SMILES: Fc1cc(Oc2ccnc(Br)c2)cc(F)c1Cl] | ¹H NMR (400 MHz, CDCl₃) δ = 8.29 (d, J = 5.6 Hz, 1H), 7.06 (d, J = 2.4 Hz, 1H), 6.88 (dd, J = 2.0, 5.8 Hz, 1H), 6.81- 6.74 (m, 2H). |
| | 2-bromo-4-(4-chloro-3,5- | |
| | difluorophenoxy)pyridine | |
| 1-50 | 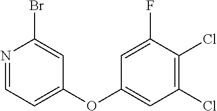 [SMILES: Fc1cc(Oc2ccnc(Br)c2)cc(Cl)c1Cl] | ¹H NMR (400 MHz, CDCl₃) δ = 8.30 (d, J = 5.6 Hz, 1H), 7.09-7.06 (m, 1H), 7.06 (d, J = 2.0 Hz, 1H), 6.91-6.85 (m, 2H). |
| | 2-bromo-4-(3,4-dichloro-5- | |
| | fluorophenoxy)pyridine | |
| 1-51 | 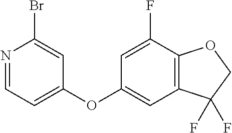 [SMILES: Fc1cc(Oc2ccnc(Br)c2)cc2c1OCC2(F)F] | ¹H NMR (400 MHz, DMSO-d₆) δ = 8.28 (d, J = 5.6 Hz, 1H), 7.63 (dd, J = 2.0, 11.2 Hz, 1H), 7.56-7.51 (m, 1H), 7.21 (d, J = 2.4 Hz, 1H), 7.02 (dd, J = 2.4, 5.6 Hz, 1H), 5.00 (t, J = 16.4 Hz, 2H). |
| | 2-bromo-4-((3,3,7-trifluoro-2,3- | |
| | dihydrobenzofuran-5-yl)oxy)pyridine | |
| 1-52 | 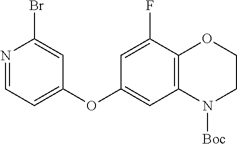 [SMILES: CC(C)(C)OC(=O)N1CCOc2c(F)cc(Oc3ccnc(Br)c3)cc21] | ¹H NMR (400 MHz, DMSO-d₆) δ = 8.27 (d, J = 5.6 Hz, 1H), 7.52 (br s, 1H), 7.14 (d, J = 2.4 Hz, 1H), 7.07-6.98 (m, 2H), 4.37-4.27 (m, 2H), 3.91-3.83 (m, 2H), 1.44 (s, 9H). |
| | tert-butyl 6-((2-bromopyridin-4- | |
| | yl)oxy)-8-fluoro-2,3-dihydro-4H- | |
| | benzo[b][1,4]oxazine-4-carboxylate | |
| 1-53 | 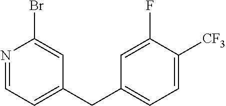 [SMILES: Fc1cc(Cc2ccnc(Br)c2)ccc1C(F)(F)F] | ¹H NMR (400 MHz, DMSO-d₆) δ = 8.40 (d, J = 5.2 Hz, 1H), 7.84 (s, 1H), 7.74 (t, J = 8.0 Hz, 1H), 7.49 (d, J = 12.0 Hz, 1H), 7.34 (d, J = 8.0 Hz, 1H), 7.19 (dd, J = 1.2, 5.2 Hz, 1H), 4.56-4.50 (m, 2H), 4.47-4.40 (m, 2H), 4.33-4.25 (m, 2H), 4.15 (s, 2H), 2.51 (s, 3H), 1.30 (t, J = 7.0 Hz, 3H). |
| | 2-bromo-4-(3-fluoro-4- | |
| | (trifluoromethyl)benzyl)pyridine | |
| 1-54 | 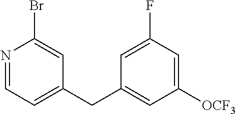 [SMILES: Fc1cc(Cc2ccnc(Br)c2)cc(OC(F)(F)F)c1] | ¹H NMR (400 MHz, DMSO-d₆) δ = 8.30 (d, J = 4.8 Hz, 1H), 7.63 (s, 1H), 7.36 (dd, J = 1.2, 5.2 Hz, 1H), 7.32-7.28 (m, 1H), 7.28-7.23 (m, 2H), 4.05 (s, 2H). |
| | 2-bromo-4-(3-fluoro-5- | |
| | (trifluoromethoxy)benzyl)pyridine | |
| 1-55 | 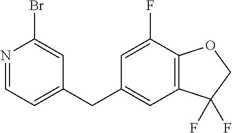 [SMILES: Fc1cc(Cc2ccnc(Br)c2)cc2c1OCC2(F)F] | ¹H NMR (400 MHz, DMSO-d₆) δ ppm 8.28 (d, J = 5.2 Hz, 1H), 7.63-7.59 (m, 1H), 7.55-7.47 (m, 2H), 7.36 (dd, J = 1.2, 5.2 Hz, 1H), 4.97-4.86 (m, 2H), 3.99 (s, 2H). |
| | 2-bromo-4-((3,3,7-trifluoro-2,3- | |
| | dihydrobenzofuran-5- | |
| | yl)methyl)pyridine | |

### C. Compounds

### Example 1. Synthesis of (R)-7-(4-(3-(difluoromethyl)-5-fluorobenzyl)pyridin-2-yl)-6-methyl-6,7-dihydroimidazo[1,5-a]pyrazin-8(5H)-one (Compound 1)

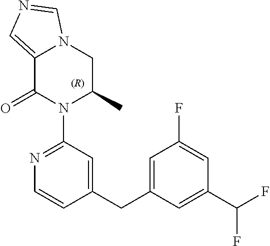

Step 1: To a stirred solution of methyl 1H-imidazole-5-carboxylate (CAS: 17325-26-7) (5.0 g, 39.6 mmol, 1.0 equiv) and tert-butyl (R)-(1-hydroxypropan-2-yl)carbamate (CAS: 1063391-86-0) (10.40 g, 59.47 mmol, 1.5 equiv) in THF (50 mL) at −40° C., was added triphenylphosphine (20.77 g, 325.8 mmol, 2.0 equiv) portion wise and DIAD (16.03 g, 79.29 mmol, 2.0 equiv) drop-wise over 15 min. The resulting reaction mixture was stirred at RT for 8 h. After completion of the reaction as indicated by TLC (50% EtOAc in hexane), the reaction mixture was poured into water (50 mL) and extracted with EtOAc (3×500 mL). The organic layers were dried over Na₂SO₄ and concentrated under reduced pressure to afford the crude compound. The crude compound was purified by normal phase column chromatography using (100-200 silica, 45% EtOAc in hexane) to afford methyl (R)-1-(2-((tert-butoxy carbonyl)amino)propyl)-1H-imidazole-5-carboxylate as a white solid (2.0 g, 17% yield). ¹H NMR (400 MHz, d₆-DMSO): δ ppm 7.74 (s, 1H), 7.58 (s, 2H, obscured by PPh₃O), 4.39-4.35 (m, 1H), 3.99-3.96 (m, 1H), 3.86-3.82 (m, 1H), 3.79 (s, 3H), 1.27 (s, 9H), 1.05 (d, J=6.4 Hz, 3H); LCMS-01 (Method-A): Rt 1.15 min, m/z 283.8 [M+H]⁺.

Step 2: To a stirred solution of methyl (R)-1-(2-((tert-butoxy carbonyl)amino) propyl)-1H-imidazole-5-carboxylate (2.0 g, 7.05 mmol, 1.0 equiv) in DCM (20 mL) at 0° C., was added 4 M HCl in dioxane (10.0 mL) dropwise and the reaction mixture was stirred at RT for 4 h. After completion of the reaction as indicated by TLC (10% MeOH in DCM), the reaction mixture was directly concentrated under reduced pressure to afford the crude compound as a hydrochloride salt. The crude hydrochloride salt was triturated with a mixture of diethyl ether (20 mL) and pentane (40 mL) to afford methyl (R)-1-(2-aminopropyl)-1H-imidazole-5-carboxylate hydrochloride (as a white solid (0.8 g, 61% yield). ¹H NMR (400 MHz, d₆-DMSO): δ ppm 9.12 (s, 1H), 8.29 (s, 1H), 7.64-7.55 (m, 2H), 4.76 (dd, J=14.0, 8.4 Hz, 1H), 4.58 (dd, J=14.0, 4.4 Hz, 1H), 3.87 (s, 3H), 3.70-3.69 (m, 1H), 1.27 (d, J=6.4 Hz, 3H). LCMS-01 (Method-A): Rt 0.35 min, m/z 183.8 [M+H]⁺.

Step 3: To a stirred solution of methyl (R)-1-(2-aminopropyl)-1H-imidazole-5-carboxylate (0.80 g, 4.36 mmol, 1.0 equiv) in methanol (80 mL), was added NaOMe (1.17 g, 21.8 mmol, 5.0 equiv) portion wise and the reaction mixture was stirred at room temperature for 6 h under N₂ atmosphere. After completion of the reaction as indicated by TLC (10% MeOH in DCM), the reaction mixture was filtered through a Celite bed and the bed was washed with methanol (3× 10 mL). The filtrate was concentrated under reduced pressure to afford the crude compound. The crude compound was purified by normal phase column chromatography (230-400 silica, gradient 0-10% MeOH in DCM) to afford (R)-6-methyl-6,7-dihydroimidazo[1,5-a]pyrazin-8(5H)-one as a white solid (0.3 g, 45% yield). ¹H NMR (400 MHz, d₆-DMSO): δ ppm 7.94 (brs, 1H), 7.80 (s, 1H), 7.43 (s, 1H), 4.30-4.28 (m, 1H), 3.86-3.79 (m, 2H), 1.17 (d, J=6.0 Hz, 3H). LCMS-01 (Method-A): Rt 0.24 min, m/z 151.8 [M+H]⁺.

Step 4: To a stirred solution of (R)-6-methyl-6,7-dihydroimidazo[1,5-a]pyrazin-8(5H)-one (0.11 g, 0.73 mmol, 1.0 equiv) and 2-chloro-4-(3-(difluoromethyl)-5-fluorobenzyl)pyridine (0.20 g, 0.73 mmol, 1.0 equiv) in 1,4-dioxane (3.0 mL), was added Cs₂CO₃ (0.47 g, 1.47 mmol, 2.0 equiv) and the reaction mixture was purged with N₂ for 15 min. The catalyst XantPhos-PdG₃ (0.07 g, 0.07 mmol, 0.1 equiv) was added and the reaction mixture was stirred at 90° C. for 6 h. After completion of the reaction as indicated by TLC (5% MeOH in DCM), the reaction mixture was filtered through a Celite bed. The filtrate was diluted with water (10 mL) and then extracted with DCM (3×10 mL). The combined organic layers were dried over Na₂SO₄ and concentrated under reduced pressure to afford the crude. The crude compound was purified by flash column chromatography (silica 230-400, gradient 0-5% MeOH in DCM) to afford (R)-7-(4-(3-(difluoro methyl)-5-fluorobenzyl)pyridin-2-yl)-6-methyl-6,7-dihydroimidazo[1,5-a]pyrazin-8(5H)-one as an off-white solid (0.075 g, 14% yield). ¹H NMR (400 MHz, d₆-DMSO): δ ppm 8.38 (d, J=5.2 Hz, 1H), 7.94 (s, 1H), 7.83 (s, 1H), 7.67 (s, 1H), 7.38-7.29 (m, 3H), 7.02-6.88 (m, 2H) (—CHF₂ merged proton), 5.11-5.09 (m, 1H), 4.36 (s, 2H), 4.09 (s, 2H), 1.25 (d, J=6.8 Hz, 3H). LCMS-01 (Method-A): Rt 1.95 min, m/z 387.55 [M+H]⁺.

### Example 2. Synthesis of 7-(6-(3-(difluoromethyl)-5-fluorobenzyl)pyrimidin-4-yl)-1-methyl-6,7-dihydroimidazo[1,5-a]pyrazin-8(5H)-one (Compound 2)

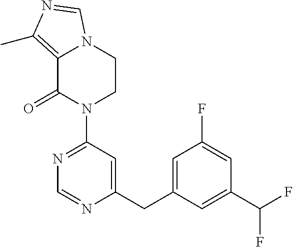 [SMILES: Cc1ncn2c1C(=O)N(c1cc(Cc3cc(F)cc(C(F)F)c3)ncn1)CC2]

To a stirred solution of 4-chloro-6-(3-(difluoromethyl)-5-fluorobenzyl)pyrimidine (0.100 g, 0.367 mmol, 1.0 equiv) and 1-methyl-6,7-dihydroimidazo[1,5-a]pyrazin-8(5H)-one (0.038 g, 0.257 mmol, 0.7 equiv) in 1,4-dioxane (2.0 mL), was added Cs₂CO₃ (0.358 g, 1.10 mmol, 3.0 equiv) and the reaction mixture was purged with N₂ for 10 min. Ruphos-Pd G3 (0.061 g, 0.073 mmol, 0.2 equiv) was added and the reaction mixture was heated at 90° C. for 4 h. After completion of the reaction as indicated by TLC (5% MeOH in DCM as a mobile phase), the reaction mixture was diluted with water (10 mL) and extracted with EtOAc (2×15 mL). The combined organic layers were dried over Na₂SO₄ and concentrated under reduced pressure to afford the crude compound. The crude was purified by reverse phase column chromatography (C18 silica, gradient 0-15% ACN in water) to afford the title compound (as a white solid (0.020 g, 14% yield); ¹H NMR (400 MHz, d₆-DMSO): δ ppm 8.93 (d, J=1.2 Hz, 1H), 8.13 (d, J=1.2 Hz, 1H), 7.83 (s, 1H), 7.40-7.37 (m, 2H), 7.31 (d, J 5=8.4 Hz, 1H), 7.03 (t, J=55.6 Hz, 1H), 4.45-4.42 (m, 2H), 4.33-4.30 (m, 2H), 4.19 (s, 2H), 2.42 (s, 3H); LCMS-02 (Method-A): Rt 1.83 min, m/z 388.2 [M+H]⁺.

### Example 3. Synthesis of 7-(6-(3-fluoro-5-(trifluoromethyl)benzyl) pyrimidin-4-yl)-6,7-dihydroimidazo[1,5-a]pyrazin-8(5H)-one (Compound 3)

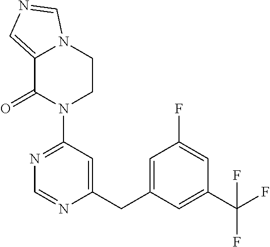 [SMILES: O=C1c2cncn2CCN1c1cc(Cc2cc(F)cc(C(F)(F)F)c2)ncn1]

To a stirred solution of 4-chloro-6-(3-fluoro-5-(trifluoromethyl)benzyl) pyrimidine (0.100 g, 0.344 mmol, 1.0 equiv) and 6,7-dihydroimidazo[1,5-a]pyrazin-8(5H)-one (0.032 g, 0.24 mmol, 0.7 equiv) in 1,4-dioxane (2.0 mL), was added Cs₂CO₃ (0.332 g, 11.0 mmol, 3.0 equiv). The reaction mixture was purged with N₂ for 10 min. The catalyst XantPhos-PdG₃ (0.032 g, 0.034 mmol, 0.1 equiv) was added and the reaction mixture was heated at 90° C. for 4 h. After completion of the reaction as indicated by TLC (5% MeOH in DCM as a mobile phase), the reaction mixture was diluted with water (15 mL) and extracted with EtOAc (3×10 mL). The combined organic layers were dried over Na₂SO₄ and concentrated under reduced pressure to afford the crude compound. The crude was purified by normal phase column chromatography (230-400 silica, gradient 0-4% MeOH in DCM) to afford 7-(6-(3-fluoro-5-(trifluoromethyl)benzyl) pyrimidin-4-yl)-6,7-dihydroimidazo[1,5-a]pyrazin-8(5H)-one as a light brown solid (0.037 g, 27% yield); ¹H NMR (400 MHz, d₆-DMSO): δ ppm 8.95 (s, 1H), 8.09 (s, 1H), 7.99 (s, 1H), 7.75 (s, 1H), 7.61 (s, 1H), 7.56 (t, J=8.8 Hz, 2H), 4.48-4.46 (m, 2H), 4.40-4.37 (m, 2H), 4.26 (s, 2H); LCMS-02 (Method-A): Rt 1.98 min, m/z 392.1 [M+H]⁺.

### Example 4. Synthesis of 7-(2-(3-(difluoromethyl)-5-fluorobenzyl)-2H-pyrazolo[4,3-c]pyridin-4-yl)-3-(methyl-d₃)-6,7-dihydroimidazo[1,5-a]pyrazin-8(5H)-one (Compound 4)

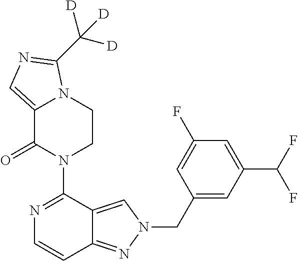 [SMILES: [2H]C([2H])([2H])c1ncc2n1CCN(c1nccc3nn(Cc4cc(F)cc(C(F)F)c4)cc13)C2=O]

Step 1: A stirred solution of 6,7-dihydroimidazo[1,5-a]pyrazin-8(5H)-one ((5.0 g, 36 mmol, 1.0 equiv) in DMF (150 mL) under nitrogen atmosphere was cooled to 0° C. and sodium hydride (60% dispersion in mineral oil) (2.18 g, 54.7 mmol, 1.5 equiv) was added portion wise and stirred for 10 min. 4-Methoxybenzyl chloride (11.4 g, 72.9 mmol, 2.0 equiv) was added dropwise and the reaction mixture was stirred at room temperature for 1 h. After completion of the reaction as indicated by TLC (5% MeOH in DCM), the reaction mixture was poured into cold water (30 mL) and extracted with EtOAc (5×50 mL). The combined organic layers were dried over sodium sulphate and concentrated under reduced pressure to afford the crude compound. The crude compound was triturated with EtOAc (30 mL) to afford 7-(4-methoxybenzyl)-6,7-dihydroimidazo[1,5-a]pyrazin-8(5H)-one as a white solid (6.0 g, 63% yield); ¹H NMR (400 MHz, d₆-DMSO): δ ppm 8.19 (s, 1H), 7.70 (s, 1H), 7.25 (d, J=8.8 Hz, 2H) 6.91 (d, J=8.8 Hz, 2H), 4.59 (s, 2H) 4.27 (t, J=6.0 Hz, 2H), 3.73 (s, 3H), 3.59 (t, J=6.0 Hz, 2H). LCMS-01 (Method-A): Rt 1.07 min, m/z 257.9 [M+H]⁺.

Step 2: Note: Two parallel reactions (2.99 g×2) were performed: To each stirred solution of 7-(4-methoxybenzyl)-6,7-dihydroimidazo[1,5-a]pyrazin-8(5H)-one (2.99 g, 11.6 mmol, 1.0 equiv) in MeCN (90 mL), was added N-bromosuccinimide (2.07 g, 11.6 mmol, 1.0 equiv) and reaction mixtures stirred at room temperature for 16 h. After completion of the reactions as indicated by TLC (5% MeOH in DCM), the reaction mixtures were concentrated under reduced pressure to afford the crude compound. The crude compounds from each batch were combined and purified by normal phase column chromatography (100-200 silica, gradient 60% EtOAc in hexane) to afford the 2 separated regioisomers. The first eluted regio-isomer appearance was a white solid (2.4 g, 30% yield) and the second eluted regio-isomer appearance was a white solid (2.6 g, 33% combined yield across 2 parallel reactions).

1-bromo-7-(4-methoxybenzyl)-6,7-dihydroimidazo[1,5-a]pyrazin-8(5H)-one (Desired isomer): ¹H NMR (400 MHz, d₆-DMSO): δ ppm 7.83 (s, 1H), 7.24 (d, J=8.4 Hz, 2H), 6.91 (d, J=8.4 Hz, 2H) 4.56 (s, 2H) 4.22 (t, J=6.0 Hz, 2H), 3.73 (s, 3H), 3.57 (t, J=5.6 Hz, 2H). LCMS-01 (Method-A): Rt 1.30 min, m/z 335.8 [M+H]⁺.

3-bromo-7-(4-methoxybenzyl)-6,7-dihydroimidazo[1,5-a]pyrazin-8 (5H)-one (undesired Isomer): ¹H NMR (400 MHz, d₆-DMSO): δ ppm 7.50 (s, 1H), 7.25 (d, J=8.8 Hz, 2H), 6.90 (d, J=8.4 Hz, 2H) 4.59 (s, 2H) 4.13 (t, J=6.0 Hz, 2H), 3.73 (s, 3H), 3.60 (t, J=6.4 Hz, 2H). LCMS-02 (Method-A): Rt 1.82 min, m/z 338.2 [M+2]⁺.

Step 3: Note: Two parallel batches (1.3 g×2) were performed: Each stirred solution of 1-bromo-7-(4-methoxybenzyl)-6,7-dihydroimidazo[1,5-a]pyrazin-8(5H)-one ((1.3 g, 3.9 mmol, 1.0 equiv) in dry THF (39 mL), was cooled to −78° C. n-BuLi (1.6 M in hexane, 2.9 mL, 1.2 equiv) was added dropwise, then the mixtures stirred for 5 min. A THF (1 mL) solution of CD₃I (CAS: 865-50-9) (0.67 g, 4.6 mmol, 1.2 equiv) was added slowly at −78° C. and the reaction mixtures stirred for 1 h at the same temperature. After 1 hour the reaction temperature was allowed to increase to 20° C. and the reactions stirred again for 30 min. Completion of the reactions was indicated by TLC (2% MeOH in DCM). The reaction mixtures were quenched with water (20 mL) and extracted with EtOAc (4×50 mL). The combined organic layers were dried over sodium sulphate and concentrated under reduced pressure to afford the crudes. The crude compound from both batches was combined and purified by normal phase column chromatography (100-200 silica, gradient 0-1.5% MeOH in DCM) to afford 7-(4-methoxybenzyl)-3-(methyl-d₃)-6,7-dihydroimidazo[1,5-a]pyrazin-8(5H)-one as a yellow gum (0.45 g, 21% combined yield across 2 parallel batches). ¹H NMR (400 MHz, d₆-DMSO): δ ppm 7.37 (s, 1H), 7.23 (d, J=8.4 Hz, 2H), 6.90 (d, J=8.4 Hz, 2H) 4.58 (s, 2H) 4.13-4.00 (m, 2H), 3.73 (s, 3H) 3.62-3.49 (m, 2H). LCMS-02 (Method-A): Rt 1.53 min, m/z 275.3 [M+H]⁺.

Step 4: To a stirred solution of 7-(4-methoxybenzyl)-3-(methyl-ď3)-6,7-dihydroimidazo[1,5-a]pyrazin-8(5H)-one (0.45 g, 1.6 mmol, 1.0 equiv) in TFA (9.0 mL) was added triflic acid (2.25 mL) at room temperature. The reaction mixture was stirred at 70° C. for 3 h. After completion of the reaction as indicated by TLC (10% MeOH in DCM), the reaction mixture was cooled to room temperature and neutralized with saturated sodium bicarbonate solution (110 mL). The aqueous layer was washed with diethyl ether (2×50 mL) and directly lyophilized to afford the crude compound. The crude was purified by normal phase column chromatography (100-200 silica, gradient 100% EtOAc then switch to 0-6% MeOH in DCM) to afford 3-(methyl-d₃)-6,7-dihydroimidazo[1,5-a]pyrazin-8(5H)-one as a yellow solid (0.14 g, 56% yield) ¹H NMR (400 MHz, d₆-DMSO): δ ppm 7.80 (brs, 1H), 7.32 (s, 1H) 4.04-4.01 (m, 2H), 3.48-3.46 (m, 2H). LCMS-02 (Method-A): Rt 0.24 min, m/z 155.1 [M+H]⁺.

Step 5: Note: Two parallel batches (0.055 g×2) were performed: To each dry sealed tube, was added 3-(methyl-d₃)-6,7-dihydroimidazo[1,5-a]pyrazin-8(5H)-one (0.055 g, 0.35 mmol, 1.0 equiv), 4-bromo-2-(3-(difluoromethyl)-5-fluorobenzyl)-2H-pyrazolo[4,3-c]pyridine (0.12 g, 0.35 mmol, 1.0 equiv), Pd(OAc)₂ (0.015 g, 0.07 mmol, 0.2 equiv), PCy₃ (0.039 g, 0.14 mmol, 0.4 equiv), CuI (0.13 g, 0.71 mmol, 2.0 equiv), Cs₂CO₃ (0.23 g, 0.71 mmol, 2.0 equiv) and anhydrous 1,4-dioxane (4.4 mL). The resulting reaction mixtures were stirred for 3 min at RT and heated up to 110° C. for 4 h in a preheated oil bath. After completion of the reactions as indicated by TLC (5% MeOH in DCM), the reaction mixtures were filtered through Celite beds and the beds washed with EtOAc (50 mL). The filtrates from both reactions were combined and concentrated under reduced pressure. The obtained crude was triturated with diethyl ether/pentane (20 mL: 30 mL) and then further purified by Prep HPLC purification (Mobile Phase: (A) 0.05% Formic acid in water (B) MeCN: Water (80:20) and column (Waters X-Bridge Prep C8 (250 mm×19 mm×5 μm)) to afford 7-(2-(3-(difluoromethyl)-5-fluorobenzyl)-2H-pyrazolo[4,3-c]pyridin-4-yl)-3-(methyl-d₃)-6,7-dihydroimidazo[1,5-a]pyrazin-8(5H)-one as a white solid (0.009 g, 3% combined yield across 2 parallel batches). ¹H NMR (400 MHz, d₆-DMSO): δ ppm 8.60 (s, 1H), 8.05 (d, J=6.0 Hz, 1H) 7.65 (brs, 1H), 7.44-7.39 (m, 4H), 7.04 (t, J=55 Hz, 1H), 5.77 (s, 2H), 4.36 (broad, 2H, splitting not well resolved), 4.29 (broad, 2H splitting not well resolved); LCMS-02 (Method-A): Rt 1.70 min, m/z 430.5 [M+H]⁺.

### Example 5. Synthesis of 1-(difluoromethyl)-7-(1-(3-(difluoromethyl)-5-fluorobenzyl)-1H-pyrazolo[4,3-c]pyridin-4-yl)-6,7-dihydroimidazo[1,5-a]pyrazin-8(5H)-one (Compound 5)

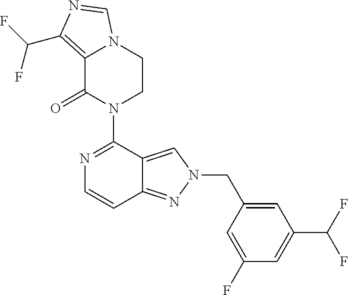 [SMILES: O=C1c2c(C(F)F)ncn2CCN1c1nccc2nn(Cc3cc(F)cc(C(F)F)c3)cc12]

Note: Two parallel reactions (2×0.1 g scale) were performed: To each stirred solution of 1-(difluoro methyl)-7-(4-methoxybenzyl)-6,7-dihydroimidazo[1,5-a]pyrazin-8(5H)-one (0.079 g, 0.42 mmol, 1.5 equiv) and 4-bromo-2-(3-(difluoro methyl)-5-fluorobenzyl)-2H-pyrazolo[4,3-c]pyridine (0.100 g, 0.28 mmol, 1.0 equiv) in 1,4-dioxane (1.0 mL) at RT under N₂ atmosphere, was added Cs₂CO₃ (0.273 g, 0.84 mmol, 3.0 equiv), tricyclohexylphosphine (0.031 g, 0.11 mmol, 0.4 equiv), and CuI (0.107 g, 0.56 mmol, 2.0 equiv) at room temperature. The catalyst Pd(OAc)₂ (0.013 g, 0.055 mmol, 0.2 equiv) was added, and the reaction mixture was stirred at 120° C. for 2 h. After completion of the reactions as indicated by TLC (5% MeOH in DCM as a mobile phase), the reaction mixtures were filtered through a celite bed and, the filtrate diluted with water (20 mL) and then extracted with DCM (3× 20 mL). The combined organic layers were dried over sodium sulphate and concentrated under reduced pressure to afford the crude. The crude compound was purified by Prep-HPLC purification (Mobile Phase: (A) 0.05% formic acid in H₂O (B) CH₃CN and Column (Waters X-Bridge Prep C8 (250 mm×19 mm 5 μm)) to afford the title compound as a pale-yellow solid (0.010 g, 7.59% yield). ¹H NMR (400 MHz, d₄-MeOD): δ 8.62 (s, 1H), 8.13 (d, J=6.4 Hz, 1H), 8.03 (s, 1H), 7.50 (d, J=6.0 Hz, 1H), 7.49-7.17 (m, 4H), 7.44-7.17 (t, J=56.0 Hz, 1H), 5.77 (s, 2H), 4.55-4.51 (m, 4H). LCMS-08 (Method-A): 1.907 min, m/z 463.15 [M+H]⁺.

### Example 6. Synthesis of 7-(2-(3-fluoro-5-(trifluoromethyl)benzyl)-2H-pyrazolo[4,3-c]pyridin-4-yl)-1-methoxy-6,7-dihydroimidazo[1,5-a]pyrazin-8(5H)-one (Compound 6)

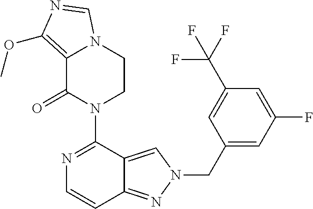 [SMILES: COc1ncn2c1C(=O)N(c1nccc3nn(Cc4cc(F)cc(C(F)(F)F)c4)cc13)CC2]

Note: Two parallel reactions (0.1 g×2) were performed: To each stirred solution of 4-bromo-2-(3-fluoro-5-(trifluoromethyl)benzyl)-2H-pyrazolo[4,3-c]pyridine (0.10 g, 0.27 mmol, 1.0 equiv) in 1,4-dioxane (1.0 mL), was added 1-methoxy-6,7-dihydroimidazo[1,5-a]pyrazin-8(5H)-one (0.045 g, 0.27 mmol, 1.0 equiv), CuI (0.102 g, 0.53 mmol, 2.0 equiv), PCy₃ (0.015 g, 0.053 mmol, 0.2 equiv) and anhydrous Cs₂CO₃ (0.261 g, 0.80 mmol, 3.0 equiv) at room temperature. The reaction mixtures were stirred at RT with N₂ gas atmosphere for 15 min. Pd(OAc)₂ (0.0060 g, 0.027 mmol, 0.1 equiv) was added and the reaction mixtures were heated up to 120° C. for 2 h in the microwave. After completion of the reactions as indicated by TLC (5% MeOH in DCM), the reaction mixtures were filtered through a Celite bed, the filtrate diluted with water (20 mL) and then extracted with EtOAc (2×20 mL). The organic layers from both reactions were combined, dried over Na₂SO₄ and concentrated under reduced pressure to afford the crude compound. The crude was purified by Prep HPLC (Mobile Phase: (A) 0.05% Formic acid in Merck water (B) MeCN and column (Xselect CSH Phenyl Hexyl (250× 19 mm×5 μm)) to afford 7-(2-(3-fluoro-5-(trifluoromethyl)benzyl)-2H-pyrazolo[4,3-c]pyridin-4-yl)-1-methoxy-6,7-dihydroimidazo[1,5-a]pyrazin-8(5H)-one as a white solid (0.0041 g, 1% combined yield over 2 parallel reactions). ¹H NMR (400 MHz, do-DMSO): δ ppm 8.61 (s, 1H), 8.01 (d, J=6.4 Hz, 1H), 7.69-7.66 (m, 3H), 7.53 (d, J=9.2 Hz, 1H), 7.37 (d, J=6.4 Hz, 1H), 5.80 (s, 2H), 4.32-4.31 (m, 4H), 3.92 (s, 3H). LCMS-02 (Method-A): Rt 1.98 min, m/z 461.6 [M+H]⁺.

### Example 7. Synthesis of 2-(3-(difluoromethyl)-5-fluorobenzyl)-4-(5,6-dihydroimidazo[1,5-a]pyrazin-7 (8H)-yl)-2H-pyrazolo[4,3-c]pyridine (Compound 7)

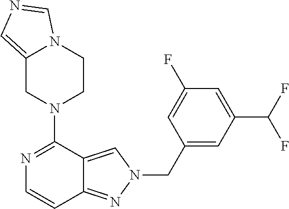 [SMILES: Fc1cc(Cn2cc3c(N4CCn5cncc5C4)nccc3n2)cc(C(F)F)c1]

To a stirred solution of 4-bromo-2-(3-(difluoromethyl)-5-fluorobenzyl)-2H-pyrazolo[4,3-c]pyridine (0.1 g, 0.280 mmol, 1.0 equiv) in 1,4-dioxane (1.0 mL), was added 5,6,7,8-tetrahydroimidazo[1,5-a]pyrazine (CAS: 297172-19-1) (0.069 g, 0.561 mmol, 2.0 equiv) and NaOfBu (0.080 g, 0.84 mmol, 3.0 equiv) at room temperature. The mixture was then purged with N₂ gas for 15 mins. The ligand Davephos (0.022 g, 0.056 mmol, 0.2 equiv) and catalyst Pd₂(dba)₃ (0.025, 0.028 mmol, 0.1 equiv) was added and the reaction mixture was heated at 80° C. for 3 h. The completion of the reaction was indicated by TLC (5% MeOH in DCM as mobile phase), the reaction mixture was diluted with water (20 mL) and extracted with EtOAc (3× 20 mL). The combined organic layers were dried over Na₂SO₄ and concentrated under reduced pressure to afford the crude compound. The crude compound was purified by reverse phase column chromatography (C-18 silica, 35% ACN in water) to afford the title compound as a white solid (0.037 g, 33% yield). ¹H NMR (400 MHz, d₆-DMSO): δppm 9.07 (s, 1H), 7.70 (d, J=6.4 Hz, 1H), 7.65 (s, 1H), 7.41-7.38 (m, 3H), 7.04 (t, J=55.2 Hz, 1H), 6.83-6.82 (m, 2H), 5.69 (s, 2H), 5.01 (s, 2H), 4.26-4.24 (m, 2H), 4.17-4.14 (m, 2H). LCMS-02 (Method A): Rt 1.58 min, m/z 399.5 [M+H]⁺.

### Example 8. Synthesis of 7-(6-(3-(difluoromethyl)-5-fluorobenzyl)pyridin-2-yl)-6,7-dihydroimidazo[1,5-a]pyrazin-8(5H)-one (Compound 8)

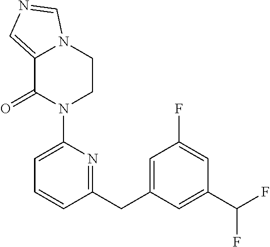 [SMILES: O=C1c2cncn2CCN1c1cccc(Cc2cc(F)cc(C(F)F)c2)n1]

To a stirred solution of 2-chloro-6-(3-(difluoromethyl)-5-fluorobenzyl)pyridine (0.2 g, 0.736 mmol, 1.0 equiv) in 1,4-dioxane (2.0 mL) was added 6,7-dihydroimidazo[1,5-a]pyrazin-8(5H)-one (0.162 g, 1.10 mmol, 1.5 equiv) and Cs₂CO₃ (1.20 g, 3.68 mmol, 5.0 equiv) at room temperature then purge with N₂ gas through the reaction mixture for 15 min. The catalyst XantPhos-PdG₃ (0.135 g, 0.147 mmol, 0.2 equiv) was added and the reaction mixture was heated at 80° C. for 5 h. The completion of the reaction was monitored by TLC (5% MeOH in DCM as mobile phase), the reaction mixture was diluted with water (20 mL) and then extracted with EtOAc (3×20 mL). The combined organic layers were dried over Na₂SO₄ and concentrated under reduced pressure to afford the crude compound. The crude was purified by reverse phase column chromatography (C-18 silica gel, gradient 40% CH₃CN in water) to afford the title compound as an off-white solid (0.093 g, 34% yield). ¹H NMR (400 MHz, d₆-DMSO): δ ppm 7.95 (s, 1H), 7.80-7.74 (m, 2H), 7.66 (s, 1H), 7.41 (s, 1H) 7.37 (d, J=9.6 Hz, 1H), 7.28 (d, J=9.2 Hz, 1H), 7.17-7.14 (m, 1H), 7.03 (t, J=55.6 Hz, 1H), 4.37-4.36 (m, 4H), 4.16 (s, 2H). LCMS-02 (Method A): Rt 1.94 min, m/z 373.5 [M+H]⁺.

### Example 9. Synthesis of 7-(4-(3-(difluoromethyl)-5-fluorobenzyl)pyridin-2-yl)-3-(methyl-d₃)-6,7-dihydroimidazo[1,5-a]pyrazin-8(5H)-one (Compound 9)

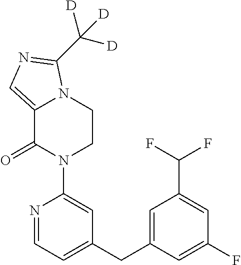 [SMILES: [2H]C([2H])([2H])c1ncc2n1CCN(c1cc(Cc3cc(F)cc(C(F)F)c3)ccn1)C2=O]

Step 1: To a stirred solution of 6,7-dihydroimidazo[1,5-a]pyrazin-8(5H)-one (7.7 g, 56.2 mmol, 1.0 equiv) in DMF (115 mL) under N₂ atmosphere, was cooled to 0° C. then sodium hydride (60% dispersion in mineral oil) (3.3 g, 84.3 mmol, 1.5 equiv) was added portion wise. The 4-methoxybenzyl chloride (CAS: 824-94-2) (13.0 g, 84.3 mmol, 1.5 equiv) was added dropwise and the reaction mixture was stirred at room temperature for 2 h. After completion of the reaction as indicated by TLC (5% MeOH in DCM), the reaction mixture was poured onto ice (200 g) which resulted in precipitation, the solid was filtered and washed with cold water (50 mL) and hexane (200 mL). The solid obtained from filtration was dried under vacuum to afford the crude compound, the crude was further triturated with EtOAc (20 mL) to afford 7-(4-methoxybenzyl)-6,7-dihydroimidazo[1,5-a]pyrazin-8(5H)-one as a white solid (8.8 g, 60% yield). ¹H NMR (400 MHz, d₆-DMSO): δ ppm 7.81 (s, 1H), 7.49 (s, 1H), 7.24 (d, J=8.8 Hz, 2H) 6.90 (d, J=8.4 Hz, 2H), 4.58 (s, 2H) 4.21 (t, J=6.0 Hz, 2H), 3.73 (s, 3H), 3.55 (t, J=5.6 Hz, 2H) LCMS-02 (Method-A): Rt 1.52 min, m/z 258.3 [M+H]⁺.

Step 2: Two parallel batches (4.4 g×2) were performed: To each stirred solution of 7-(4-methoxybenzyl)-6,7-dihydroimidazo[1,5-a]pyrazin-8(5H)-one (4.4 g, 17.1 mmol, 1.0 equiv) in MeCN (132 mL), was added N-bromosuccinimide (3 g, 17.1 mmol, 1.0 equiv) at room temperature and the reactions stirred for 16 h. After completion of the reactions as indicated by TLC (100% EtOAc), the two batches were combined and concentrated under vacuum to afford the crude. The crude was purified by normal phase column chromatography (100-200 silica, gradient 60% EtOAc in hexane) to afford the two separated regio-isomers. The first eluted regio-isomer was a pale-yellow solid (3.3 g, 28% yield). The second eluted regio-isomer was also a pale-yellow solid (3.5 g, 30% combined yield across 2 parallel batches) and characterized as the desired isomer 1-bromo-7-(4-methoxybenzyl)-6,7-dihydroimidazo[1,5-a]pyrazin-8(5H)-one (based on NOE analysis. ¹H NMR (400 MHz, d6-DMSO): δ ppm 7.83 (s, 1H), 7.25 (d, J=8.8 Hz, 2H), 6.91 (d, J=8.4 Hz, 2H) 4.56 (s, 2H) 4.22 (t, J=6.0 Hz, 2H), 3.73 (s, 3H), 3.57 (t, J=6.0 Hz, 2H), LCMS: Rt 1.57 min, m/z 337.9 [M+H]⁺.

Characterisation of the undesired isomer: 3-bromo-7-(4-methoxybenzyl)-6,7-dihydroimidazo[1,5-a]pyrazin-8(5H)-one: ¹H NMR (400 MHz, d₆-DMSO): δ ppm 7.53 (s, 1H), 7.24 (d, J=8.4 Hz, 2H), 6.90 (d, J=8.4 Hz, 2H) 4.58 (s, 2H) 4.11 (t, J=6.4 Hz, 2H), 3.73 (s, 3H) 3.59 (t, J=6.0 Hz, 2H), LCMS: Rt 1.63 min, m/z 338.0 [M+H]⁺ for ⁸¹Br.

Step 3: Note: Two parallel reactions (2×1.75 g) were performed: To each stirred solution of 1-bromo-7-(4-methoxybenzyl)-6,7-dihydroimidazo[1,5-a]pyrazin-8(5H)-one (1.75 g, 4.99 mmol, 1.0 equiv) in dry THF (50 mL), cooled to −78° C. under nitrogen atmosphere was added n-BuLi (1.6 M, 3.7 mL, 1.2 equiv) dropwise. The reaction mixtures were stirred for 2 min. To each reaction mixture was slowly added a THF solution (1 mL), of Iodomethane-d₃ (CAS: 865-50-9) (0.87 g, 6.0 mmol, 1.2 equiv). The reactions were stirred at −78° C. for 1 h, then allowed to warm to room temperature in 30 min. After completion of the reactions, as indicated by TLC (2% MeOH in DCM), the reaction mixtures were quenched with water (20 mL), combined and concentrated under a vacuum to afford the crude compound. The crude compound was purified by normal phase column chromatography using (100-200 silica, gradient 0-1.5% MeOH in DCM) to afford 7-(4-methoxybenzyl)-3-(methyl-d₃)-6,7-dihydroimidazo[1,5-a]pyrazin-8(5H)-one as a white solid (0.41 g, 15% yield). ¹H NMR (400 MHz, d₆-DMSO): δ ppm 7.37 (s, 1H), 7.23 (d, J=8.4 Hz, 1H), 6.90 (d, J=8.4 Hz, 2H) 4.58 (s, 2H) 4.07 (t, J=5.6 Hz, 2H), 3.73 (s, 3H) 3.53 (t, J=6.0 Hz, 2H), LCMS-02 (Method-A): Rt 1.62 min, m/z 275.4 [M+H]⁺.

Step 4: To a stirred solution of 7-(4-methoxybenzyl)-1-(methyl-d₃)-6,7-dihydroimidazo[1,5-a]pyrazin-8(5H)-one (0.41 g, 1.49 mmol, 1.0 equiv) in the mixture of TFA (4.1 mL) and triflic acid (2.0 mL), was heated at 70° C. for 3 h. After completion of the reaction as indicated by TLC (10% MeOH in DCM), the reaction mixture was cooled to room temperature and diluted with 15% MeOH in DCM (300 mL), the solution was neutralized with the addition of solid potassium carbonate. The resulting suspension was filtered through a Celite bed and the bed was washed with 15% MeOH in DCM (70 mL). The filtrate was collected and concentrated under a vacuum to get the crude compound. The crude was purified by normal phase column chromatography (100-200 silica, gradient 0-5% MeOH in DCM) to afford 3-(methyl-d₃)-6,7-dihydroimidazo[1,5-a]pyrazin-8(5H)-one as white solid (0.2 g, 91% yield) ¹H NMR (400 MHz, d₆-DMSO): δ ppm 7.84 (brs, 1H), 7.36 (s, 1H) 4.06 (t, J=6.0 Hz, 2H), 3.50-3.48 (m, 2H) LCMS: Rt 0.28 min, m/z 155.2 [M+H]⁺.

Step 5: To a dry sealed tube, was added 3-(methyl-d₃)-6,7-dihydroimidazo[1,5-a]pyrazin-8(5H)-one (0.2 g, 1.29 mmol, 1.0 equiv), 2-bromo-4-(3-(difluoromethyl)-5-fluorobenzyl)pyridine (0.4 g, 1.29 mmol, 1.0 equiv), Cs₂CO₃ (0.84 g, 2.59 mmol, 2.0 equiv) and 1,4-dioxane (14.0 mL) then purged with N₂ (g) for 15 min. The catalyst XantPhos-PdG₃ was added and the reaction mixture was heated at 90° C. under stirring for 4 h. After completion of the reaction as indicated by TLC (2% MeOH in DCM) the reaction mixture was poured into water (10 mL) and extracted with EtOAc (3×20 mL). The combined organic layers were dried over sodium sulphate and concentrated under reduced pressure to afford the crude compound. The crude compound was first purified by normal phase column chromatography (100-200 silica, gradient 0-1.5% MeOH in DCM) to afford the title compound with enriched purity and it was further purified by Prep HPLC purification (Mobile Phase: (A) 0.05% Formic acid In Merck water (B) MeCN: Methanol [50:50] and column Xselect CSH Phenyl Hexyl (250 mm×19 mm×5 μm) to afford 7-(4-(3-(difluoromethyl)-5-fluorobenzyl)pyridin-2-yl)-3-(methyl-d₃)-6,7-dihydroimidazo[1,5-a] pyrazin-8(5H)-one as a white solid. (0.064 g, 12% yield); ¹H NMR (400 MHz, d₆-DMSO): δ ppm 8.36 (d, J=5.2 Hz, 1H), 7.80 (s, 1H) 7.55 (s, 1H), 7.38-7.36 (m, 2H), 7.30 (d, J=8.8 Hz, 1H), 7.13 (d, J=5.2 Hz, 1H), 7.02 (t, J=55.6 Hz, 1H), 4.34-4.31 (m, 2H), 4.25-4.22 (m, 2H) 4.10 (s, 2H); ¹H NMR (400 MHz, d₄-MeOD): δ ppm 8.37 (d, J=4.8 Hz, 1H), 7.75 (s, 1H) 7.65 (s, 1H), 7.30 (s, 1H), 7.20-7.18 (m, 2H) 7.11 (d, J=8.8 Hz, 1H), 6.77 (t, J=56.0 Hz, 1H), 4.41 (d, J=6.0 Hz, 2H), 4.30 (d, J=6.0 Hz, 2H), 4.12 (s, 2H), LCMS: Rt 1.90 min, m/z 390.5 [M+H]⁺.

### Example 10. Synthesis of 7-(4-(3-(difluoromethyl)-5-fluorobenzyl)pyridin-2-yl)-3-(methyl-d₃)-6,7-dihydroimidazo[1,5-a]pyrazin-8(5H)-one-1-d (Compound 10)

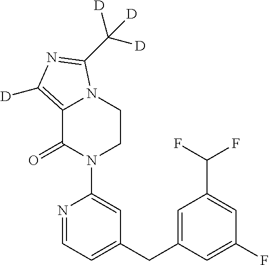 [SMILES: [2H]c1nc(C([2H])([2H])[2H])n2c1C(=O)N(c1cc(Cc3cc(F)cc(C(F)F)c3)ccn1)CC2]

Step 1: To a stirred solution 6,7-dihydroimidazo[1,5-a]pyrazin-8(5H)-one (5.0 g, 36.45 mmol, 1.0 equiv) in DMF (150 mL), was cooled to 0° C. and sodium hydride (60%) (2.83 g, 72.9 mmol, 2.0 equiv) was added portion wise then stirred for 10 min at same temperature. To this reaction mixture, 4-methoxybenzyl chloride (CAS: 824-94-2) (8.55 g, 54.7 mmol, 1.5 equiv) was added dropwise and stirred at room temperature for 2 h. After completion of the reaction as indicated by TLC (5% MeOH in DCM), the reaction mixture was poured into cold water (200 mL), the precipitate observed was filtered and washed with cold water (30 mL) then dried under vacuum to get crude compound. The crude was triturated with EtOAc (30 mL) to afford 7-(4-methoxybenzyl)-6,7-dihydroimidazo[1,5-a] pyrazin-8(5H)-one a white solid (6.1 g, 65% yield); ¹H NMR (400 MHz, d₆-DMSO): δ ppm 7.82 (s, 1H), 7.50 (s, 1H), 7.24 (d, J=8.8 Hz, 2H) 6.90 (d, J=8.1 Hz, 2H), 4.59 (s, 2H) 4.22 (t, J=6.0 Hz, 2H), 3.73 (s, 3H), 3.55 (t, J=6.0 Hz, 2H) LCMS-02 (Method-A): RT 1.48 min, m/z 258.3 [M+H]⁺.

Step 2: To a stirred solution 7-(4-methoxybenzyl)-6,7-dihydroimidazo[1,5-a] pyrazin-8(5H)-one (6.1 g, 24 mmol, 1.0 equiv) in MeCN (183 mL), was added NBS (4.21 g, 23.7 mmol, 1.0 equiv) and the resulting reaction mixture was stirred at room temperature for 16 h. After completion of the reaction as indicated by TLC (using 100% EtOAc), the reaction mixture was concentrated under reduced pressure to afford the crude compound. The crude was purified by normal phase column chromatography (100-200 silica, gradient 70% EtOAc in hexane) to afford the two separated regioisomers. The first eluted regio-isomer from column was a white solid (2.1 g, 26% yield) and second eluted regio-isomer was also a white solid (2.4 g, 30%).

Isomer 1: (Desired): ¹H NMR (400 MHz, d₆-DMSO): δ ppm 7.83 (s, 1H), 7.25 (d, J=8.4 Hz, 2H), 6.91 (d, J=8.4 Hz, 2H) 4.56 (s, 2H) 4.22 (t, J=5.6 Hz, 2H), 3.73 (s, 3H) 3.57 (t, J=5.2 Hz, 2H); LCMS-02 (Method-A): RT 1.74 min, m/z 338.3 [M+2]⁺.

Isomer 2: (undesired): LCMS-02 (Method-A): RT 1.73 min, m/z 338.3 [M+H]⁺ for ⁸¹Br.

Step 3: Note: Two parallel batches (1.2 g×2) were performed: Each stirred solution of 1-bromo-7-(4-methoxybenzyl)-6,7-dihydroimidazo[1,5-a]pyrazin-8(5H)-one (Isomer 1) (2.4 g, 7.1 mmol, 1.0 equiv) in dry THF (72 mL), was cooled to −78° C. and n-BuLi (1.6 [M], 5.2 mL, 1.2 equiv) was added dropwise under nitrogen atmosphere. The reaction mixtures were stirred for 5 min. A THF solution (0.5 mL) of Iodomethane-d₃ (CAS: 865-50-9) (1.24 g, 8.56 mmol, 1.2 equiv) was added slowly to each reaction mixture and stirred at −78° C. for 1 h. The reaction mixtures were allowed to warm to 20° C. and stirred for 30 min. After completion of the reactions as indicated by TLC (2% MeOH in DCM), the reaction mixtures were quenched with water (2 mL), combined and concentrated under reduced pressure. The combined crude material was purified by normal phase column chromatography using (100-200 silica, gradient 0-1.5% MeOH in DCM) to afford 7-(4-methoxybenzyl)-3-(methyl-d₃)-6,7-dihydroimidazo[1,5-a]pyrazin-8(5H)-one-1-d as a yellow solid (0.4 g, 20% yield). H NMR (400 MHz, d₆-DMSO): δ ppm 7.23 (d, J=8.4 Hz, 2H), 6.90 (d, J=8.4 Hz, 2H) 4.58 (s, 2H) 4.03 (t, J=7.2 Hz, 2H), 3.73 (s, 3H) 3.53 (t, J=6.0 Hz, 2H), LCMS-02 (Method-A): RT 1.50 min, m/z 275.4 [M+H]⁺.

Step 4: To a stirred solution of 7-(4-methoxybenzyl)-3-(methyl-d₃)-6,7-dihydroimidazo[1,5-a]pyrazin-8(5H)-one-1-d (0.4 g, 1.45 mmol, 1.0 equiv) in TFA (8.0 mL) and triflic acid (2.0 mL), was stirred at 70° C. for 3 h. After completion of the reaction as indicated by TLC (5% MeOH in DCM), the reaction mixture was cooled to room temperature and diluted with 30% MeOH in DCM (150 mL). The solution was neutralized with the addition of solid K₂CO₃, then resulting suspension was filtered through a celite bed and the bed was washed with 30% MeOH in DCM (100 mL). The filtrate was collected and concentrated under reduced pressure to afford the crude product. The crude was purified by normal phase column chromatography (100-200 silica, gradient 100% EtOAc then 0-4% MeOH in DCM) to afford 3-(methyl-d₃)-6,7-dihydroimidazo[1,5-a]pyrazin-8(5H)-one-1-d as yellow solid (0.18 g, 80% yield) ¹H NMR (400 MHz, d₆-DMSO): δ ppm 7.95 (brs, 1H), 4.06 (t, J=5.6 Hz, 2H), 3.52-3.48 (broad, 2H). LCMS-02 (Method-A): RT 0.23 min, m/z 155.1 [M+H]⁺.

Step 5: Note: Three parallel reactions (0.05 g×3) were performed: To each dry sealed tube, was added 3-(methyl-d₃)-6,7-dihydroimidazo[1,5-a]pyrazin-8(5H)-one-1-d (0.15 g, 0.972 mmol, 1.0 equiv), 2-chloro-4-(3-(difluoro-methyl)-5-fluorobenzyl)pyridine (0.3 g, 0.96 mmol, 1.0 equiv), Pd(OAc)₂ (0.087 g, 0.36 mmol, 0.4 equiv), Davephos (0.3 g, 0.75 mmol, 0.8 equiv), CuI (0.3 g, 1.92 mmol, 2.0 equiv), Cs₂CO₃ (0.63 g, 1.92 mmol, 2.0 equiv) and 1,4-dioxane (12 mL). The reaction mixtures were stirred at 110° C. for 6 h under the microwave irradiation. After completion of the reactions as indicated by TLC (5% MeOH in DCM), the reaction mixtures were filtered through a celite bed and the beds washed with EtOAc. The filtrates from each reaction were combined, washed with water, dried over sodium sulphate and concentrated under reduced pressure to afford the crude product. The crude was triturated with pentane (5 mL) and 10% solution of diethyl ether in pentane (20 mL), the residue was further purified by prep HPLC (Mobile Phase: (A) 0.05% formic acid in MERCK H₂O (B) MeCN: H₂O (90:10) and column X-select CSH phenyl hexyl (250 mm×19 mm×5 μm)) to afford 7-(4-(3-(difluoromethyl)-5-fluorobenzyl)pyridin-2-yl)-3-(methyl-d₃)-6,7-dihydroimidazo[1,5-a]pyrazin-8(5H)-one-1-d as a yellow solid (0.007 g, 2% combined yield across 3 parallel reactions). ¹H NMR (400 MHz, d₆-DMSO): δ ppm 8.38 (d, J=5.2 Hz, 1H), 7.80 (s, 1H) 7.38-7.30 (m, 3H), 7.17-6.88 (m, 3H, CHF₂ signal merge with other aromatic protons), 4.40-4.35 (broad, 2H, splitting not resolve well), 4.32-4.26 (broad, 2H, splitting not resolve well) 4.11 (s, 2H), LCMS-02 (Method-A): RT 1.80 min, m/z 390.4 [M+H]⁺.

### Example 11. Synthesis of 7-(2-(3-(difluoromethyl)-5-fluorobenzyl)pyridin-4-yl)-6,7-dihydroimidazo[1,5-a]pyrazin-8 (5H)-one (Compound 11)

### General Method 1:

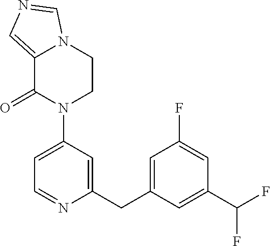 [SMILES: O=C1c2cncn2CCN1c1ccnc(Cc2cc(F)cc(C(F)F)c2)c1]

To a stirred solution of aryl halide (1.0 equiv) and amide (0.7-1.0 equiv) in 1,4-dioxane (0.9-1.8 mL), was added K₂CO₃ (4.0 equiv) and CuI (0.2 equiv). The reaction mixture was purged with N₂ for 10 min. The DMEDA (0.4 equiv) was added and the reaction mixture heated at the temperature specified in Table 2 for the time specified in Table 2. After completion of the reaction as indicated by TLC, the reaction mixture was diluted with water (10-30 mL) and extracted with EtOAc (3× 10-20 mL). The combined organic layers were dried over Na₂SO₄ and concentrated under reduced pressure to afford the crude compound. The crude was purified as specified in Table 2 to afford the title compound.

TABLE 2
Compounds prepared by General Method 1

| Structure/Comp. No | Rxn cond. | Purification | ¹H NMR | LCMS |
|---|---|---|---|---|
| 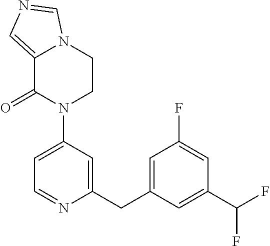 [SMILES: O=C1c2cncn2CCN1c1ccnc(Cc2cc(F)cc(C(F)F)c2)c1] | 1,4- dioxane (0.9 mL) 120 °C for 24 h | 230-400 silica, 0-5% MeOH in DCM | ¹H NMR (400 MHz, d₆-DMSO): δ ppm 8.49 (d, J = 5.6 Hz, 1H), 7.94 (s, 1H), 7.65 (brs, 1H), 7.49 (d, J = 2.0 Hz, 1H), 7.38-7.32 (m, 3H), 7.27 (d, J = 9.6 Hz, 1H), 7.02 (t, J = 55.6 Hz, 1H), 4.43-4.40 (m, 2H), 4.19-4.16 (m, 4H) | LCMS-08 (Method-A): Rt 1.60 min, m/z 373.2 [M + H]⁺. |
| 11 | | | | |
| 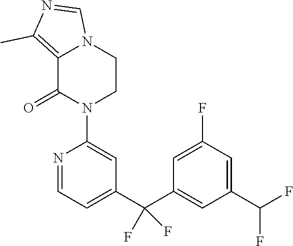 [SMILES: Cc1ncn2c1C(=O)N(c1cc(C(F)(F)c3cc(F)cc(C(F)F)c3)ccn1)CC2] | 1,4- dioxane (1.0 mL) 120 °C for 24 h | 230-400 silica, 0-7% MeOH in DCM | ¹H NMR (400 MHz, d₆₋DMSO): δ ppm 8.60 (d, J = 5.2 Hz, 1H), 8.17 (s, 1H), 7.81 (s, 1H), 7.76 (d, J = 9.2 Hz, 1H), 7.70-7.67 (m, 2H), 7.44 (d, J = 4.8 Hz, 1H), 7.12 (t, J = 55.2 Hz, 1 H), 4.37-4.34 (m, 4H), 2.41 (s, 3H). | LCMS-02 (Method-A): Rt 1.99 min, m/z 423.1 [M + H]⁺. |
| 12 | | | | |
| 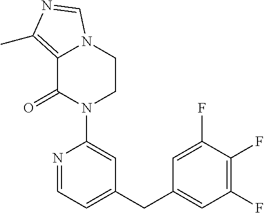 [SMILES: Cc1ncn2c1C(=O)N(c1cc(Cc3cc(F)c(F)c(F)c3)ccn1)CC2] | 1,4- dioxane (1.0 mL) 110 °C for 16 h | 230-400 silica, 0-5% MeOH in DCM | ¹H NMR (400 MHz, d₆₋DMSO): δ ppm 8.34 (d, J = 5.2 Hz, 1H), 7.80-7.79 (m, 2H signal merge), 7.31 (t, J = 7.6 Hz, 2H), 7.11 (d, J = 4.8 Hz, 1 H), 4.30 (s, 4H), 4.00 (s, 2H), 2.40 (s, 3H) | LCMS-08 (Method-A): Rt 1.74 min, m/z 373.2 [M + H]⁺. |
| 13 | | | | |
| 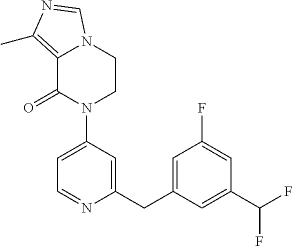 [SMILES: Cc1ncn2c1C(=O)N(c1ccnc(Cc3cc(F)cc(C(F)F)c3)c1)CC2] | 1,4- dioxane (0.9 mL) 120 °C for 24 h | 230-400 silica, 0-5% MeOH in DCM | ¹H NMR (400 MHz, d₆-DMSO): δ ppm 8.47 (d, J = 5.6 Hz, 1H), 7.78 (s, 1H), 7.49 (d, J = 2.0 Hz, 1H), 7.38-7.26 (m, 4H), 7.02 (t, J = 55.6 Hz, 1H), 4.36-4.33 (m, 2H), 4.17 (s, 2H), 4.15-4.12 (m, 2H), 2.38 (s, 3H) | LCMS-02 (Method-A): Rt 1.65 min, m/z 387.1 [M + H]⁺. |
| 14 | | | | |
| 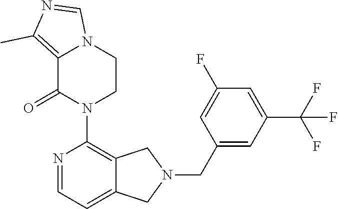 [SMILES: Cc1ncn2c1C(=O)N(c1nccc3c1CN(Cc1cc(F)cc(C(F)(F)F)c1)C3)CC2] | 1,4- dioxane (1.8 mL) 120 °C for 24 h | 230-400 silica, 0-5% MeOH in DCM) | ¹H NMR (400 MHz, d₆₋DMSO): δ ppm 8.33 (d, J = 4.8 Hz, 1H), 7.78 (s, 1H), 7.59-7.54 (m, 3H), 7.26 (d, J = 4.8 Hz, 1H), 4.32 (t, J = 5.2 Hz, 2H), 4.14 (t, J = 5.2 Hz, 2H), 4.00 (s, 2H), 3.96 (s, H), 3.80 (s, 2H), 2.37 (s, 3H) | LCMS-08 (Method-A): Rt 1.61 min, m/z 446.2 [M + H]⁺. |
| 15 | | | | |
| 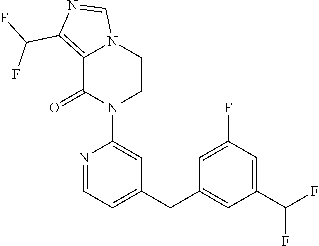 [SMILES: O=C1c2c(C(F)F)ncn2CCN1c1cc(Cc2cc(F)cc(C(F)F)c2)ccn1] | 1,4- dioxane (2.0 mL) 120 °C for 24 h | C18 silica, 0-20% MeCN in H₂O | ¹H NMR (400 MHz, d₆₋DMSO): δ ppm 8.39 (d, J = 5.2 Hz, 1H), 8.06 (s, 1H), 7.85 (s, 1H), 7.40- 7.37 (m, 2H), 7.31-7.29 (m, 2H), 7.19-7.16 (m, 1H), 7.01 (t, J = 55.6 Hz, 1H), 4.42-4.40 (m, 4H), 4.12 (s, 2H) | LCMS-02 (Method-A): Rt 2.21 min, m/z 423.1 [M + H]⁺. |
| 16 | | | | |
| 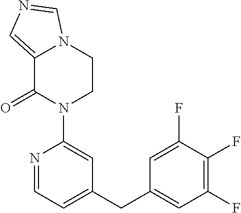 [SMILES: O=C1c2cncn2CCN1c1cc(Cc2cc(F)c(F)c(F)c2)ccn1] | 1,4- dioxane (2.0 mL) 120 °C for 24 h | 230-400 silica, 0-6% MeOH in DCM | ¹H NMR (400 MHz, d₆₋DMSO): δ ppm 8.36 (d, J = 5.2 Hz, 1H), 7.96 (brs, 1H), 7.88 (s, 1H), 7.67 (brs, 1H), 7.33-7.29 (m, 2H), 7.14 (d, J = 4.8 Hz, 1H), 4.38- 4.33 (m, 4H), 4.01 (s, 2H) | LCMS-02 (Method-A): Rt 1.87 min, m/z 359.1 [M + H]⁺. |
| 17 | | | | |
| 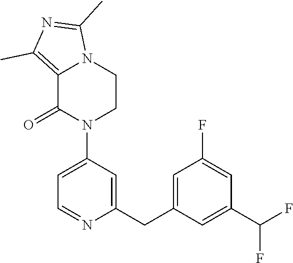 [SMILES: Cc1nc(C)n2c1C(=O)N(c1ccnc(Cc3cc(F)cc(C(F)F)c3)c1)CC2] | 1,4- dioxane (0.9 mL) 120 °C for 24 h | 230-400 silica, 0-4% MeOH in DCM | ¹H NMR (400 MHz, d₆₋DMSO): δ ppm 8.45 (d, J = 5.6 Hz, 1H), 7.48 (s, 1H), 7.38-7.26 (m, 4H), 7.02 (t, J = 55.2 Hz, 1H), 4.21- 4.11 (m, 6H), 2.34 (s, 3H), 2.31 (s, 3H) | LCMS-02 (Method A): Rt 1.66 min, m/z 401.20 [M + H]⁺. |
| 18 | | | | |
| 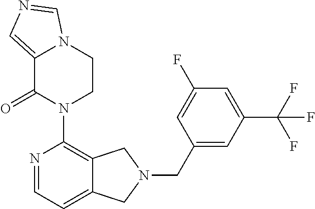 [SMILES: O=C1c2cncn2CCN1c1nccc2c1CN(Cc1cc(F)cc(C(F)(F)F)c1)C2] | 1,4- dioxane (1.8 mL) 120 °C for 24 h | 230-400 silica, 0-4% MeOH in DCM then prep-HPLC purification (Mobile Phase): (A) 0.05% formic acid in H₂O (B) MeCN and column Shim-Pack GIST C18 (250 mm × 20 mm × 5 μm) | ¹H NMR (400 MHz, d₆₋DMSO): δ ppm 8.35 (d, J = 4.4 Hz, 1H), 7.99 (s, 1H), 7.64-7.54 (m, 4H), 7.29 (d, J = 4.8 Hz, 1H), 4.39 (t, J = 4.8 Hz, 2H), 4.21 (t, J = 5.6 Hz, 2H), 4.02 (s, 4H), 3.83 (s, 2H). | LCMS-02 (Method-A): Rt 1.53 min, m/z 432.2 [M + H]⁺. |
| 19 | | | | |
| 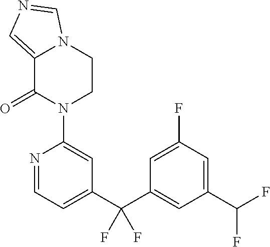 [SMILES: O=C1c2cncn2CCN1c1cc(C(F)(F)c2cc(F)cc(C(F)F)c2)ccn1] | 1,4- dioxane (2.0 mL) 120 °C for 24 h | 230-400 silica, gradient 0- 4% MeOH in DCM | ¹H NMR (400 MHz, d₆₋DMSO): δ ppm 8.62 (d, J = 5.2 Hz, 1H), 8.16 (s, 1H), 7.98 (brs, 1H), 7.76- 7.67 (m, 4H), 7.48 (d, J = 5.2 Hz, 1H), 7.12 (t, J = 55.2 Hz, 1H), 4.40 (s, 4H). | LCMS-02 (Method-A): Rt 2.19 min, m/z 409.2 [M + H]⁺. |
| 20 | | | | |
| 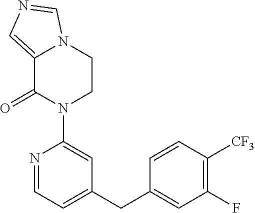 [SMILES: O=C1c2cncn2CCN1c1cc(Cc2ccc(C(F)(F)F)c(F)c2)ccn1] | 120 °C for 24 h | 230-400 silica, gradient 0- 4.5% MeOH in DCM | ¹H NMR (400 MHz, d₆₋DMSO): δ ppm 8.37 (d, J = 5.2 Hz, 1H), 7.99 (brs, 1H), 7.81 (s, 1H), 7.74 (t, J = 8.0 Hz, 1H), 7.48 (d, J = 12 Hz, 1H), 7.34 (d, J = 7.6 Hz, 1H), 7.16 (d, J = 4.8 Hz, 1H), 4.38-3.34 (m, 4H), 4.14 (s, 2H). | LCMS-02 (Method-A): Rt 2.19 min, m/z 391.1 [M + H]⁺. |
| 21 | | | | |
| 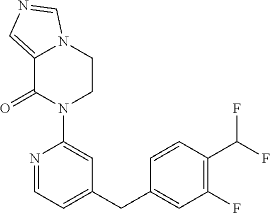 [SMILES: O=C1c2cncn2CCN1c1cc(Cc2ccc(C(F)F)c(F)c2)ccn1] | 1,4- dioxane (1.0 mL) 120 °C for 24 h | 230-400 silica, gradient 0- 4% MeOH in DCM | ¹H NMR (400 MHz, d₆₋DMSO): δ ppm 8.36 (d, J = 5.2 Hz, 1H), 7.95 (s, 1H), 7.80 (s, 1H), 7.66 (br s, 1H), 7.59 (t, J = 7.6 Hz, 1H), 7.35-7.26 (m, 2H), 7.17- 7.03 (m, 2H), 4.36 (m, 4H), 4.09 (s, 2H). | LCMS-02 (Method-A): Rt 2.03 min, m/z 373.2 [M + H]⁺. |
| 22 | | | | |

### Example 12. Synthesis of 7-(6-(3-(difluoromethyl)-5-fluorobenzyl)pyrimidin-4-yl)-6,7-dihydroimidazo[1,5-a]pyrazin-8(5H)-one (Compound 23)

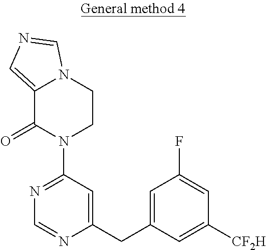 [SMILES: O=C1c2cncn2CCN1c1cc(Cc2cc(F)cc(C(F)F)c2)ncn1]

To a stirred solution of aryl halide (1.0 equiv) and amide (1.2 equiv) in 1,4-dioxane (0.7-5.0 mL), was added Cs₂CO₃ (2.0 equiv) at room temperature then the reaction mixture purged with N₂ gas for 10-15 min. The catalyst XantPhos-PdG₃ (0.2-0.3 equiv) was added and the reaction mixture stirred at 80° C. for the specified time. After completion of the reaction as indicated by TLC, the reaction mixture was diluted with water and extracted with EtOAc (×3). The combined organic layers were dried over Na₂SO₄ and concentrated under reduced pressure to afford the crude compound. The crude compound was purified as specified in Table 3 to afford the title compound.

TABLE 3
Compounds prepared using General Method 4

| | Rxn | XantPhosPd-G₃ | Purification | | |
|---|---|---|---|---|---|
| Structure/Comp. No | cond. | (eq.) | conditions | NMR | LCMS |
| 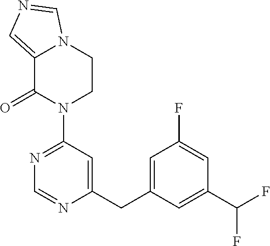 [SMILES: O=C1c2cncn2CCN1c1cc(Cc2cc(F)cc(C(F)F)c2)ncn1] | 2.0 mL 3 h | 0.2 | 230-400 silica, gradient 0-5% MeOH in DCM | ¹H NMR (400 MHz, d₆-DMSO): δ ppm 8.95 (d, J = 1.2 Hz, 1H), 8.08 (d, J = 1.2 Hz, 1H), 7.99 (s, 1H), 7.74 (d, J = 0.8 Hz, 1H), 7.40-7.31 (m, 3H), 7.03 (t, J = 55.6 Hz, 1H), 4.48-4.45 (m, 2H), 4.39-4.37 (m, 2H), 4.21 (s, 2H). | LCMS-02 (Method-A): Rt 1.93 min, m/z 374.4 [M + H]⁺. |
| 23 | | | | | |
| 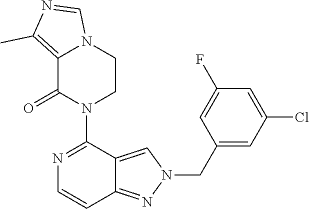 [SMILES: Cc1ncn2c1C(=O)N(c1nccc3nn(Cc4cc(F)cc(Cl)c4)cc13)CC2] | 1.0 mL 5 h | 0.1 | 230-400 silica gel, 5% MeOH in DCM | ¹H NMR (400 MHz, d₆-DMSO): δ ppm 8.59 (s, 1H), 8.04 (d, J = 6.4 Hz, 1H), 7.85 (s, 1H), 7.42 (d, J = 6.4 Hz, 2H), 7.31 (s, 1H), 7.23 (d, J = 9.6 Hz, 1H), 5.72 (s, 2H), 4.36-4.31 (m, 4H), 2.45 (s, 3H). | LCMS-02 (Method-A): Rt 1.79 min, m/z 411.4 [M + H]⁺. |
| 24 | | | | | |
| 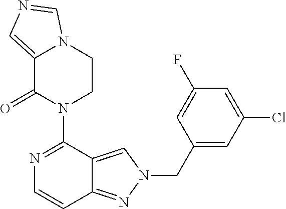 [SMILES: O=C1c2cncn2CCN1c1nccc2nn(Cc3cc(F)cc(Cl)c3)cc12] | 1.4 mL 4 h | 0.1 | 230-400 silica, 0-5% MeOH in DCM | ¹H NMR (400 MHz, d₆₋DMSO): δ ppm 8.61 (s, 1H), 8.05 (d, J = 6.0 Hz, 1H), 8.01 (s, 1H), 7.75 (s, 1H), 7.45-7.40 (m, 2H), 7.31 (s, 1H), 7.23 (d, J = 9.2 Hz, 1H), 5.71 (s, 2H), 4.44-4.37 (m, 4H). | LCMS-02 (Method-A): Rt 1.79 min, m/z 397.5 [M + H]⁺. |
| 25 | | | | | |
| 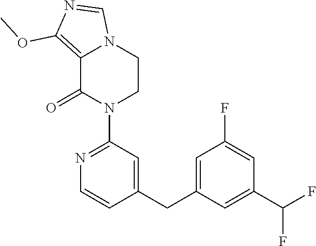 [SMILES: COc1ncn2c1C(=O)N(c1cc(Cc3cc(F)cc(C(F)F)c3)ccn1)CC2] | 5.0 mL 5 h | 0.1 | reverse phase column chromato- graphy (C18 silica, gradient 0-35% ACN in water) | ¹H NMR (400 MHz, d₆₋DMSO): δ ppm 8.32 (d, J = 5.2 Hz, 1H), 7.75 (s, 1H), 7.62 (s, 1H), 7.38- 7.33 (m, 2H), 7.30 (d, J = 8.8 Hz, 1H), 7.08 (d, J = 4.8 Hz, 1H), 7.02 (t, J = 55.6 Hz, 1H) 4.27 (s, 4H), 4.07 (s, 2H), 3.89 (s, 3H). | LCMS-02 (Method-A): Rt 2.09 min, m/z 403.5 [M + H]⁺. |
| 26 | | | | | |
| 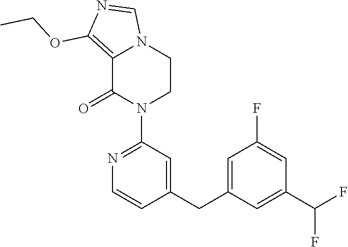 [SMILES: CCOc1ncn2c1C(=O)N(c1cc(Cc3cc(F)cc(C(F)F)c3)ccn1)CC2] | 0.7 mL 4 h | 0.1 | (230-400 silica, 4% MeOH in DCM) | ¹H NMR (400 MHz, d₆₋DMSO): δ ppm 8.32 (d, J = 4.8 Hz, 1H), 7.79 (s, 1H), 7.61 (s, 1H), 7.37- 7.35 (m, 2H), 7.30 (d, J = 8.8 Hz, 1H), 7.08 (d, J = 5.2 Hz, 1H), 7.02 (t, J = 55.6 Hz, 1H), 4.32- 4.27 (m, 6H), 4.07 (s, 2H), 1.30 (t, J = 7.2 Hz, 3H). | LCMS-08 (Method-A): Rt 1.95 min, m/z 417.2 [M + H]⁺. |
| 27 | | | | | |
| 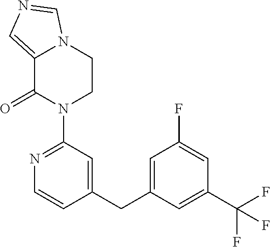 [SMILES: O=C1c2cncn2CCN1c1cc(Cc2cc(F)cc(C(F)(F)F)c2)ccn1] | 1.4 mL 5 h | 0.3 | (230-400 silica, gradient 5% MeOH in DCM) | ¹H NMR (400 MHz, d₆₋DMSO): δ ppm 8.37 (d, J = 4.8 Hz, 1H), 7.94 (s, 1H), 7.81 (s, 1H), 7.66 (s, 1H), 7.59-7.52 (m, 3H), 7.17 (d, J = 4.8 Hz, 1H), 4.36-4.33 (m, 4H), 4.15 (s, 2H). | LCMS-02 (Method-A): Rt 2.05 min, m/z 391.6 [M + H]⁺. |
| 28 | | | | | |

### Example 13. Synthesis of 3-fluoro-5-((2-(8-oxo-5,6-dihydroimidazo[1,5-a]pyrazin-7 (8H)-yl)pyridin-4-yl)methyl)benzonitrile (Compound 29)

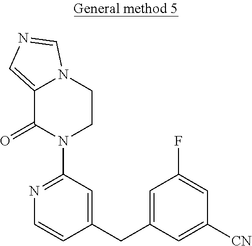 [SMILES: N#Cc1cc(F)cc(Cc2ccnc(N3CCn4cncc4C3=O)c2)c1]

To a mixture of amide (1.0 eq.), aryl halide (1.0 eq.), CuI (0.2 eq.) and K₂CO₃ (4.0 eq.) under nitrogen was added 1,4-dioxane (volume as specified in Table 4) followed by N,N′-dimethyl ethylenediamine (0.4 eq.). The reaction mixture was stirred at the specified temperature for the allotted time. Upon completion, the reaction was cooled to RT, diluted with EtOAc, filtered, washed with EtOAc, and concentrated in vacuo. The residue was purified by automated reverse phase column chromatography eluting with MeCN:H₂O (5% to 100%) using Biotage Selekt® unit to afford the title compound.

TABLE 4
Compounds prepared using general method 5

| | Rxn | | |
|---|---|---|---|
| Structure/Comp. No. | cond. | ¹H NMR/LC-MS | HRMS (ESI) |
| 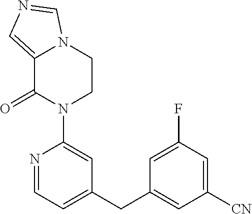 [SMILES: N#Cc1cc(F)cc(Cc2ccnc(N3CCn4cncc4C3=O)c2)c1] | 120 °C, 14 h, 16% | (400 MHz, Acetone-d6) δ 8.35 (d, J = 5.1 Hz, 1H), 7.96 (s, 1H), 7.79 (s, 1H), 7.63 (d, J = 6.8 Hz, 2H), 7.55-7.47 (m, 2H), 7.12 (dd, J = 5.1, 1.6 Hz, 1H), 4.49 (s, 4H), 4.19 (s, 2H). | m/z calcd for C₁₉H₁₅FN₅O [M + H]⁺: 348.1255; found: 348.1253. |
| 29 | | | |
| 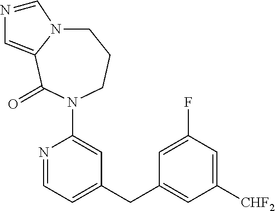 [SMILES: O=C1c2cncn2CCCN1c1cc(Cc2cc(F)cc(C(F)F)c2)ccn1] | 110 °C, 16 h, 29% | (400 MHz, Acetone-d6) δ 8.33 (d, J = 5.1 Hz, 1H), 8.11-8.07 (m, 1H), 7.71 (s, 1H), 7.52 (d, J = 1.0 Hz, 1H), 7.40 (s, 1H), 7.29 (d, J = 8 Hz, 1H), 7.24 (d J = 8.9 Hz, 1H), 7.07 (dd, J = 5.1, 1.5 Hz, 1H), 6.91 (t, J = 55.9 Hz, 1H), 4.37 (t, J = 6.9 Hz, 2H), 4.19 (t, J = 6.3 Hz, 2H), 4.16 (s, 2H), 2.30 (p, J = 6.6 Hz, 2H). | m/z calcd for C₂₀H₁₈F₃N₄O [M + H]⁺: 387.1427; found: 387.1427 |
| 30 | | | |
| 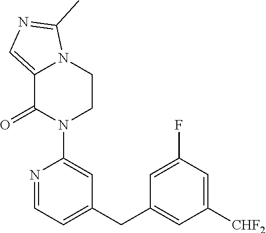 [SMILES: Cc1ncc2n1CCN(c1cc(Cc3cc(F)cc(C(F)F)c3)ccn1)C2=O] | 110 °C, 16 h, 47% | (400 MHz, Acetone-d6) δ 8.33 (d, J = 5.1 Hz, 1H), 7.96 (s, 1H), 7.56 (s, 1H), , 7.40 (s, 1H), 7.29 (d, J = 9.7 Hz, 1H), 7.27-7.20 (m, 1H), 7.08 (dd, J = 5.1, 1.5 Hz, 1H), 6.91 (t, J = 55.9 Hz, 1H), 4.49-4.46 (m, 2H), 4.34-4.31 (m, 2H), , 4.16 (s, 2H), 2.39 (s, 3H). | m/z calcd for C₂₀H₁₈F₃N₄O [M + H]⁺: 387.1427; found: 387.1425 |
| 31 | | | |
| 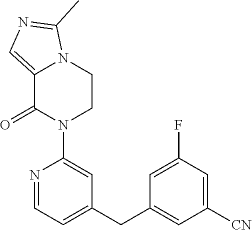 [SMILES: Cc1ncc2n1CCN(c1cc(Cc3cc(F)cc(C#N)c3)ccn1)C2=O] | 120 °C, 16 h, 23% | (400 MHz, Acetone-d6) δ 8.34 (d, J = 5.1 Hz, 1H), 7.95 (s, 1H), 7.62 (s, 1H), 7.60-7.48 (m, 3H), 7.10 (dd, J = 5.1, 1.5 Hz, 1H), 4.48 (dd, J = 6.9, 4.5 Hz, 2H), 4.32 (dd, J = 6.9, 4.5 Hz, 2H), 4.18 (s, 2H), 2.39 (s, 3H). | m/z calcd for C₂₀H₁₇FN₅O [M + H]⁺: 362.1412; found: 362.1409 |
| 32 | | | |
| 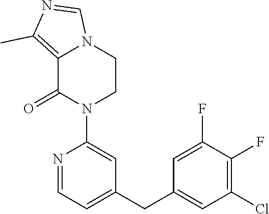 [SMILES: Cc1ncn2c1C(=O)N(c1cc(Cc3cc(F)c(F)c(Cl)c3)ccn1)CC2] | 110 °C, 16 h, 47% | (400 MHz, Acetone-d6) δ 8.32 (d, J = 4.3 Hz, 1H), 7.94 (d, J = 1.5 Hz, 1H), 7.63 (s, 1H), 7.37-7.27 (m, 2H), 7.07 (dd, J = 5.1, 1.6 Hz, 1H), 4.48-4.38 (m, 4H), 4.09 (s, 2H), 2.45 (s, 3H). | calcd for C₁₉H₁₆ClF₂N₄O [M + H]⁺: 389.0975; found: 389.0975. |
| 33 | | | |
| 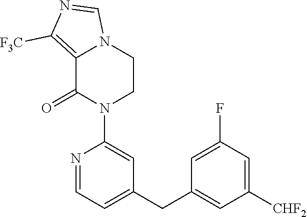 [SMILES: O=C1c2c(C(F)(F)F)ncn2CCN1c1cc(Cc2cc(F)cc(C(F)F)c2)ccn1] | 110 °C, 24 h, 56% | (400 MHz, Acetone-d6) δ 8.37 (d, J = 5.1 Hz, 1H), 8.00 (s, 1H), 7.92 (s, 1H), 7.41 (s, 1H), 7.31 (d, J = 9.7 Hz, 1H), 7.24 (d, J = 9.1 Hz, 1H), 7.13 (dd, J = 5.1, 1.6 Hz, 1H), 6.90 (t, J = 55.9 Hz, 1H), 4.60 (q, J = 2.2 Hz, 4H), 4.21 (s, 2H). | m/z calcd for C₂₀H₁₅F₆N₄O [M + H]⁺: 441.1145; found: 441.1147 |
| 34 | | | |
| 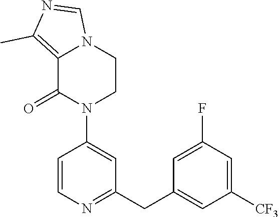 [SMILES: Cc1ncn2c1C(=O)N(c1ccnc(Cc3cc(F)cc(C(F)(F)F)c3)c1)CC2] | 120 °C, 24 h, 42% | (400 MHz, Acetone-d6) δ 8.49 (s, 1H), 7.84 (s, 1H), 7.59 (s, 1H), 7.55 (s, 1H), 7.47 (d, J = 9.6 Hz, 1H), 7.36-7.32 (m, 2H), 4.44 (d, J = 5.7 Hz, 2H), 4.35- 4.21 (m, 4H), 2.39 (s, 3H). | m/z calcd for C₂₀H₁₇F₄N₄O [M + H]⁺: 450.1333; found: 405.1325. |
| 35 | | | |
| 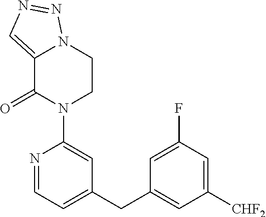 [SMILES: O=C1c2cnnn2CCN1c1cc(Cc2cc(F)cc(C(F)F)c2)ccn1] | 110 °C, 24 h, 48% | (400 MHz, Acetone-d6) δ 8.39 (d, J = 5.1 Hz, 1H), 8.19 (s, 1H), 7.96 (s, 1H), 7.40 (s, 1H), 7.30 (d, J = 9.7 Hz, 1H), 7.25 (d, J = 8.9 Hz, 1H), 7.17 (dd, J = 5.1, 1.6 Hz, 1H), 6.90 (t, J = 55.9 Hz, 1H), 4.87 (dd, J = 7.1, 4.7 Hz, 2H), 4.73- 4.67 (m, 2H), 4.20 (s, 2H). | m/z calcd for C₁₈H₁₅F₃N₅O [M + H]⁺: 374.1223; found: 374.1219 |
| 36 | | | |
| 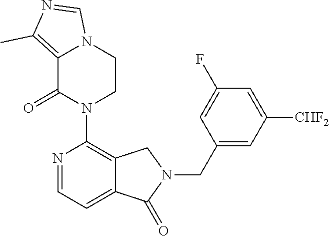 [SMILES: Cc1ncn2c1C(=O)N(c1nccc3c1CN(Cc1cc(F)cc(C(F)F)c1)C3=O)CC2] | 120 °C, 16 h, 31% | (400 MHz, Acetone-d6) δ 8.64 (d, J = 4.9 Hz, 1H), 7.67-7.60 (m, 2H), 7.45 (s, 1H), 7.36-7.25 (m, 2H), 6.91 (t, J = 55.8 Hz, 1H), 4.93 (s, 2H), 4.53 (s, 2H), 4.43 (dd, J = 7.0, 4.1 Hz, 2H), 4.38- 4.30 (m, 2H), 2.41 (s, 3H). | m/z calcd for C₂₂H₁₉F₃N₅O₂ [M + H]⁺: 442.1485; found: 442.1486. |
| 37 | | | |
| 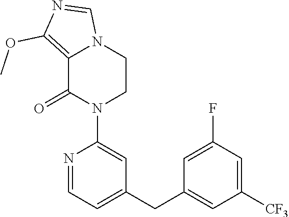 [SMILES: COc1ncn2c1C(=O)N(c1cc(Cc3cc(F)cc(C(F)(F)F)c3)ccn1)CC2] | 120 °C, 24 h, 42% | (400 MHz, DMSO-d6) δ 8.32 (d, J = 5.1 Hz, 1H), 7.77 (s, 1H), 7.61 (s, 1H), 7.59-7.48 (m, 3H), 7.10 (dd, J = 5.2, 1.5 Hz, 1H), 4.27 (s, 4H), 4.11 (s, 2H), 3.89 (s, 3H). | m/z calcd for C₂₀H₁₇F₄N₄O₂ [M + H]⁺: 421.1282; found: 421.1285 |
| 38 | | | |
| 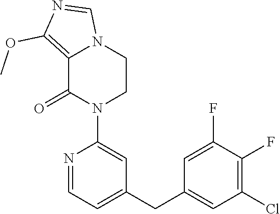 [SMILES: COc1ncn2c1C(=O)N(c1cc(Cc3cc(F)c(F)c(Cl)c3)ccn1)CC2] | 110 °C 16 h, 39% | (400 MHz, DMSO-d6) δ 8.31 (d, J = 5.1 Hz, 1H), 7.74 (s, 1H), 7.62 (s, 1H), 7.47-7.35 (m, 2H), 7.08 (dd, J = 5.1, 1.5 Hz, 1H), 4.27 (s, 4H), 3.99 (s, 2H), 3.89 (s, 3H). | m/z calcd for C₁₉H₁₆ClF₂N₄O₂ [M + H]⁺: 405.0924; found: 405.0925. |
| 39 | | | |
| 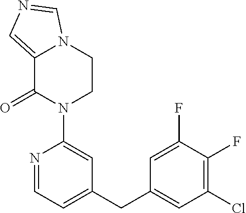 [SMILES: O=C1c2cncn2CCN1c1cc(Cc2cc(F)c(F)c(Cl)c2)ccn1] | 110 °C, 24 h, 51% | (400 MHz, Acetone-d6) δ 8.34 (d, J = 5.1 Hz, 1H), 7.96-7.92 (m, 1H), 7.79 (s, 1H), 7.65 (s, 1H), 7.36-7.28 (m, 2H), 7.10 (dd, J = 5.2, 1.5 Hz, 1H), 4.49 (s, 4H), 4.09 (s, 2H). | m/z calcd for C₁₈H₁₄ClF₂N₄O [M + H]⁺: 375.0819; found: 375.0805. |
| 40 | | | |
| 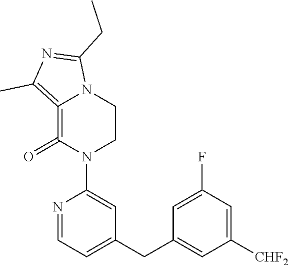 | 110 °C, 24 h, 24% | (400 MHz, Acetone-d6) δ 8.36 (d, J = 5.1 Hz, 1H), 7.97 (s, 1H), 7.41 (s, 1H), 7.30 (d, J = 9.7 Hz, 1H), 7.25 (d, J = 8.9 Hz, 1H), 7.13 (dd, J = 5.1, 1.5 Hz, 1H), 6.91 (t, J = 55.9 Hz, 1H), 4.61- 4.51 (m, 4H), 4.19 (s, 2H), 3.02 (q, J = 7.6 Hz, 2H), 2.57 (s, 3H), 1.36 (t, J = 7.5 Hz, 3H). | m/z calcd for C₂₂H₂₂F₃N₄O [M + H]⁺: 415.1740; found: 415.1740 |
| 41 | | | |
| 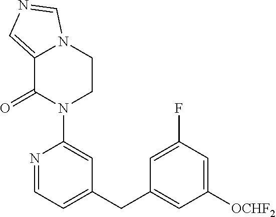 [SMILES: O=C1c2cncn2CCN1c1cc(Cc2cc(F)cc(OC(F)F)c2)ccn1] | 120 °C, 16 h, 21% | (400 MHz, Acetone-d6) δ 8.34 (d, J = 5.2 Hz, 1H), 7.95 (s, 1H), 7.79 (s, 1H), 7.64 (s, 1H), 7.26-6.86 (m, 5H), 4.49 (s, 4H), 4.11 (s, 2H). | m/z calcd for C₁₉H₁₆F₃N₄O₂ [M + H]⁺: 389.1220; found: 389.1222 |
| 42 | | | |
| 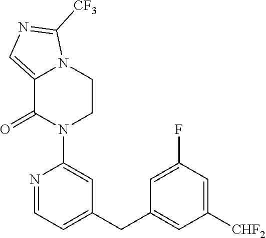 [SMILES: O=C1c2cnc(C(F)(F)F)n2CCN1c1cc(Cc2cc(F)cc(C(F)F)c2)ccn1] | 110 °C, 24 h, 56% | (400 MHz, DMSO-d6) δ 8.39 (d, J = 5.1 Hz, 1H), 7.84 (s, 1H), 7.82 (s, 1H), 7.38-7.36 (m, 2H), 7.33-7.27 (m, 1H), 7.18 (dd, J = 5.1, 1.5 Hz, 1H), 7.01 (t, J = 55.6 Hz, 1H), 4.54-4.51 (m, 2H), 4.48-4.41 (m, 2H), 4.12 (s, 2H). | m/z calcd for C₂₀H₁₅F₆N₄O [M + H]⁺: 441.1145; found: 441.1146. |
| 43 | | | |
| 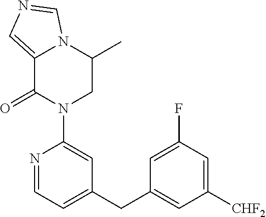 [SMILES: CC1CN(c2cc(Cc3cc(F)cc(C(F)F)c3)ccn2)C(=O)c2cncn21] | 110 °C, 24 h, 55% | (400 MHz, Acetone-d6) δ 8.34 (d, J = 5.1 Hz, 1H), 8.00 (s, 1H), 7.88 (s, 1H), 7.65 (s, 1H), 7.40 (s, 1H), 7.30 (d, J = 9.7 Hz, 1H), 7.24 (d, J = 8.5 Hz, 1H), 7.09 (dd, J = 5.2, 1.5 Hz, 1H), 6.90 (t, J = 55.9 Hz, 1H), 4.76-4.69 (m, 1H), 4.58 (dd, J = 13.6, 3.8 Hz, 1H), 4.22- 4.13 (m, 3H), 1.67 (d, J = 6.5 Hz, 3H). | m/z calcd for C₂₀H₁₈F₃N₄O [M + H]⁺: 387.1427; found: 387.1427. |
| 44 | | | |
| 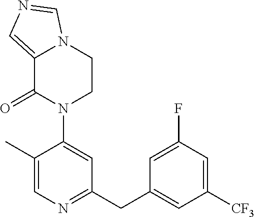 [SMILES: Cc1cnc(Cc2cc(F)cc(C(F)(F)F)c2)cc1N1CCn2cncc2C1=O] | 120 °C, 16 h, 14% | (400 MHz, Acetone-d6) δ 8.44 (s, 1H), 7.77 (s, 1H), 7.58 (s, 1H), 7.56 (s, 1H), 7.44 (dt, J = 9.7, 1.9 Hz, 1H), 7.38 (s, 1H), 7.34 (dd, J = 8.7, 2.0 Hz, 1H), 4.56 (t, J = 5.7 Hz, 2H), 4.24 (s, 2H), 4.16 (s, 2H), 2.21 (s, 3H). | m/z calcd for C₂₀H₁₇F₄N₄O [M + H]⁺: 405.1333; found: 405.1332 |
| 45 | | | |
| 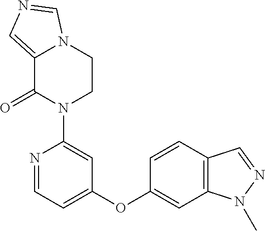 [SMILES: Cn1ncc2ccc(Oc3ccnc(N4CCn5cncc5C4=O)c3)cc21] | 120 °C, 48 h, 10% | (400 MHz, Acetone-d6) δ 8.32 (d, J = 5.7 Hz, 1H), 8.02 (d, J = 1.0 Hz, 1H), 7.85 (dd, J = 8.6, 0.7 Hz, 1H), 7.77 (s, 1H), 7.72 (d, J = 2.3 Hz, 1H), 7.59 (s, 1H), 7.42-7.41 (m, 1H), 6.98 (dd, J = 8.7, 2.0 Hz, 1H), 6.79 (dd, J = 5.7, 2.3 Hz, 1H), 4.58-4.51 (m, 2H), 4.49-4.46 (m, 2H), 4.06 (s, 3H). | m/z calcd for C₁₉H₁₇N₆O₂ [M + H]⁺: 361.1408; found: 361.1404 |
| 46 | | | |
| 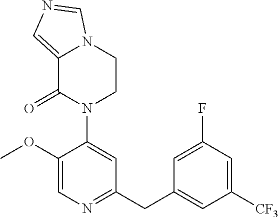 [SMILES: COc1cnc(Cc2cc(F)cc(C(F)(F)F)c2)cc1N1CCn2cncc2C1=O] | 120 °C, 24 h, 44% | (400 MHz, Acetone-d6) δ 8.40 (s, 1H), 7.76 (s, 1H), 7.56 (s, 2H), 7.44 (d, J = 10.0 Hz, 1H), 7.40 (s, 1H), 7.35 (d, J = 8.8 Hz, 1H), 4.52-4.45 (m, 2H), 4.22 (s, 2H), 4.09-4.03 (m, 2H), 3.97 (s, 3H). | m/z calcd for C₂₀H₁₇F₄N₄O₂ [M + H]⁺: 421.1282; found: 421.1280. |
| 47 | | | |
| 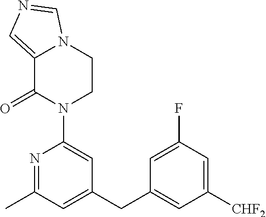 [SMILES: Cc1cc(Cc2cc(F)cc(C(F)F)c2)cc(N2CCn3cncc3C2=O)n1] | 120 °C, 48 h, 16% | (400 MHz, DMSO-d6) δ 7.97 (br s, 1H), 7.69 (br s, 1H), 7.63 (s, 1H), 7.38- 7.25 (m, 3H), 7.17-6.86 (m, 2H), 4.35 (s, 4H), 4.05 (s, 2H), 2.42 (s, 3H). | m/z calcd for C₂₀H₁₈F₃N₄O [M + H]⁺: 387.1427; found: 387.1425 |
| 48 | | | |
| 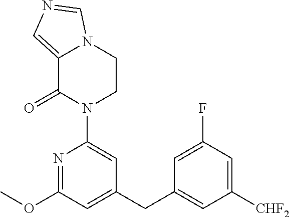 [SMILES: COc1cc(Cc2cc(F)cc(C(F)F)c2)cc(N2CCn3cncc3C2=O)n1] | 120 °C, 72 h, 26% | (400 MHz, DMSO-d6) δ 7.93 (s, 1H), 7.64 (d, J = 0.9 Hz, 1H), 7.48 (d, J = 1.1 Hz, 1H), 7.37-35 (m, 2H), 7.29 (d, J = 8.6 Hz, 1H), 7.02 (t, J = 55.6 Hz, 1H), 6.57 (s, 1H), 4.38 (s, 4H), 4.04 (s, 2H), 3.85 (s, 3H). | m/z calcd for C₂₀H₁₈F₃N₄O₂ [M + H]⁺: 403.1376; found: 403.1376 |
| 49 | | | |
| 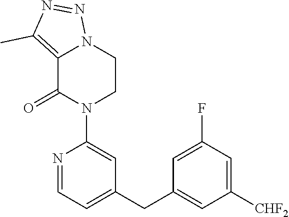 [SMILES: Cc1nnn2c1C(=O)N(c1cc(Cc3cc(F)cc(C(F)F)c3)ccn1)CC2] | 110 °C, 24 h, 63% | (400 MHz, Acetone-d6) δ 8.38 (d, J = 5.1 Hz, 1H), 7.97 (s, 1H), 7.41 (s, 1H), 7.30 (d, J = 9.6 Hz, 1H), 7.25 (d, J = 9.0 Hz, 1H), 7.15 (dd, J = 5.2, 1.5 Hz, 1H), 6.91 (t, J = 55.9 Hz, 1H), 4.81- 4.78 (m, 2H), 4.67-4.64 (m, 2H), 4.20 (s, 2H), 2.51 (s, 3H). | m/z calcd for C₁₉H₁₇F₃N₅O [M + H]⁺: 388.1380; found: 388.1382. |
| 50 | | | |
| 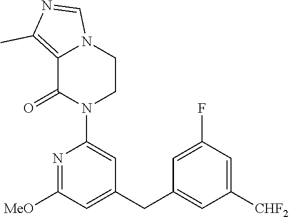 [SMILES: COc1cc(Cc2cc(F)cc(C(F)F)c2)cc(N2CCn3cnc(C)c3C2=O)n1] | 130 °C, 24 h, 22% | (400 MHz, Acetone-d6) δ 7.63 (d, J = 1.1 Hz, 1H), 7.62 (s, 1H), 7.40 (s, 1H), 7.30 (d, J = 9.7 Hz, 1H), 7.27-7.19 (m, 1H), 6.91 (t, J = 55.9 Hz, 1H), 6.47 (d, J = 1.1 Hz, 1H), 4.53-4.45 (m, 2H), 4.41 (m, 2H), 4.11 (s, 2H), 3.38 (s, 3H), 2.45 (s, 3H) | m/z calcd for C₂₁H₂₀F₃N₄O₂ [M + H]⁺: 417.1533; found: 417.1538. |
| 51 | | | |
| 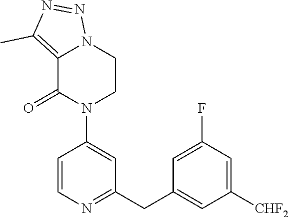 [SMILES: Cc1nnn2c1C(=O)N(c1ccnc(Cc3cc(F)cc(C(F)F)c3)c1)CC2] | 120 °C, 16 h, 55% | (400 MHz, Acetone-d6) δ 8.53 (d, J = 5.5 Hz, 1H), 7.55 (d, J = 2.2 Hz, 1H), 7.43 (s, 1H), 7.38 (dd, J = 5.5, 2.2 Hz, 1H), 7.32 (d, J = 9.8 Hz, 1H), 7.20 (d, J = 9.0 Hz, 1H), 6.89 (t, J = 55.9 Hz, 1H), 4.88-4.79 (m, 2H), 4.50-4.42 (m, 2H), 4.25 (s, 2H), 2.48 (s, 3H) | m/z calcd for C₁₉H₁₇F₃N₅O [M + H]⁺: 388.1380; found: 388.1385. |
| 52 | | | |
| 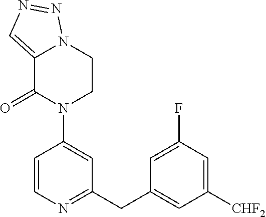 [SMILES: O=C1c2cnnn2CCN1c1ccnc(Cc2cc(F)cc(C(F)F)c2)c1] | 120 °C, 16 h, 57% | (400 MHz, Acetone-d6) δ 8.55 (d, J = 5.5 Hz, 1H), 8.18 (s, 1H), 7.55 (d, J = 2.2 Hz, 1H), 7.43 (s, 1H), 7.40 (dd, J = 5.5, 2.2 Hz, 1H), 7.31 (d, J = 9.8 Hz, 1H), 7.20 (d, J = 9.0 Hz, 1H), 6.89 (t, J = 56.0 Hz, 1H), 4.96-4.85 (m, 2H), 4.54-4.45 (m, 2H), 4.25 (s, 2H) | m/z calcd for C₁₈H₁₅F₃N₅O [M + H]⁺: 374.1223; found: 374.1229. |
| 53 | | | |
| 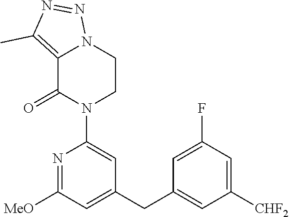 [SMILES: COc1cc(Cc2cc(F)cc(C(F)F)c2)cc(N2CCn3nnc(C)c3C2=O)n1] | 130 °C, 48 h, 28% | (400 MHz, Acetone-d6) δ 7.63 (s, 1H), 7.41 (s, 1H), 7.30 (d, J = 9.8 Hz, 1H), 7.24 (d, J = 9.0 Hz, 1H), 6.91 (t, J = 55.9 Hz, 1H), 6.55 (s, 1H), 4.80-4.77 (m, 2H), 4.72-4.69 (m, 2H), 4.14 (s, 2H), 3.90 (s, 3H), 2.50 (s, 3H). LC/MS (APCI): m/z = 418.0 [M + H]⁺, Rt 3.836 min. | |
| 54 | | | |
| 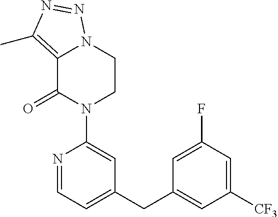 [SMILES: Cc1nnn2c1C(=O)N(c1cc(Cc3cc(F)cc(C(F)(F)F)c3)ccn1)CC2] | 120 °C, 24 h, 41% | (400 MHz, Acetone-d6) δ 8.39 (d, J = 5.2 Hz, 1H), 7.99-7.98 (m, 1H), 7.58 (s, 1H), 7.47-7.44 (m, 1H), 7.43-7.38 (m, 1H), 7.18 (dd, J = 5.1, 1.5 Hz, 1H), 4.81-4.76 (m, 2H), 4.68-4.63 (m, 2H), 4.26 (s, 2H), 2.51 (s, 3H). | m/z calcd for C₁₉H₁₆F₄N₅O [M + H]⁺: 406.1285; found: 406.1297. |
| 55 | | | |
| 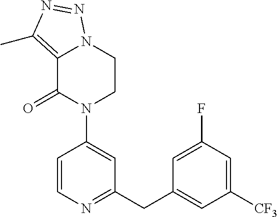 [SMILES: Cc1nnn2c1C(=O)N(c1ccnc(Cc3cc(F)cc(C(F)(F)F)c3)c1)CC2] | 120 °C, 24 h, 38% | (400 MHz, Acetone-d6) δ 8.54 (d, J = 5.5 Hz, 1H), 7.59-7.58 (m, 2H), 7.50- 7.44 (m, 1H), 7.41-7.33 (m, 2H), 4.87- 4.81 (m, 2H), 4.50-4.44 (m, 2H), 4.30 (s, 2H), 2.48 (s, 3H). | m/z calcd for C₁₉H₁₆F₄N₅O [M + H]⁺: 406.1285; found: 406.1298. |
| 56 | | | |
| 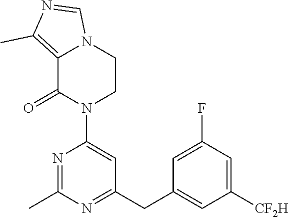 [SMILES: Cc1nc(Cc2cc(F)cc(C(F)F)c2)cc(N2CCn3cnc(C)c3C2=O)n1] | 120 °C, 24 h, 11% | (400 MHz, Acetone-d6) δ 8.01 (s, 1H), 7.67 (s, 1H), 7.46 (s, 1H), 7.38-7.33 (m, 1H), 7.25-7.20 (m, 1H), 6.91 (t, J = 55.9 Hz, 1H), 4.59-4.55 (m, 2H), 4.44-4.39 (m, 2H), 4.17 (s, 2H), 2.56 (s, 3H), 2.45 (s, 3H). | m/z calcd for C₂₀H₁₉F₃N₅O [M + H]⁺: 402.1536; found: 402.1546. |
| 57 | | | |
| 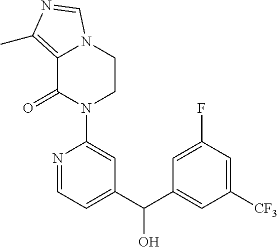 [SMILES: Cc1ncn2c1C(=O)N(c1cc(C(O)c3cc(F)cc(C(F)(F)F)c3)ccn1)CC2] | 110 °C, 16 h, 59% | (400 MHz, Acetone-d6) δ 8.36 (dd, J = 5.2, 0.8 Hz, 1H), 8.18-8.17 (m, 1H), 7.73 (s, 1H), 7.64 (s, 1H), 7.60-7.57 (m, 1H), 7.43-7.40 (m, 1H), 7.24 (dd, J = 5.2, 1.5 Hz, 1H), 6.07 (s, 1H), 5.69 (d, J = 4.0 Hz, 1H), 4.47-4.39 (m, 4H), 2.46 (s, 3H). | m/z calcd for C₂₀H₁₇F₄N₄O₂ [M + H]⁺: 421.1282; found: 421.1287. |
| 58 | | | |
| 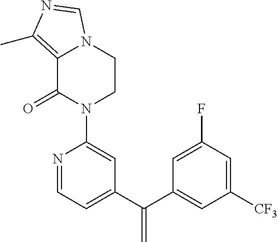 [SMILES: C=C(c1cc(F)cc(C(F)(F)F)c1)c1ccnc(N2CCn3cnc(C)c3C2=O)c1] | 110 °C, 24 h, 62% | (400 MHz, Acetone-d6) δ 8.44 (dd, J = 5.2, 0.8 Hz, 1H), 8.03-8.02 (m, 1H), 7.64 (s, 1H), 7.58-7.53 (m, 2H), 7.50- 7.47 (m, 1H), 7.13 (dd, J = 5.2, 1.6 Hz, 1H), 5.91 (d, J = 13.8 Hz, 2H), 4.53- 4.47 (m, 2H), 4.47-4.41 (m, 2H), 2.43 (s, 3H). | m/z calcd for C₂₁H₁₇F₄N₄O [M + H]⁺: 417.1333; found: 417.1348. |
| 59 | | | |
| 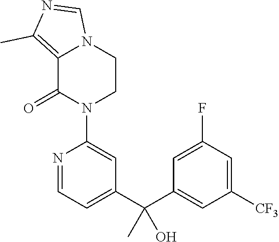 [SMILES: Cc1ncn2c1C(=O)N(c1cc(C(C)(O)c3cc(F)cc(C(F)(F)F)c3)ccn1)CC2] | 110 °C, 24 h, 59% | (400 MHz, DMSO-d6) δ 8.35 (d, J = 5.3 Hz, 1H), 8.07-8.03 (m, 1H), 7.78 (s, 1H), 7.68 (s, 1H), 7.65-7.61 (m, 1H), 7.57-7.54 (m, 1H), 7.27 (dd, J = 5.3, 1.6 Hz, 1H), 6.40 (s, 1H), 4.30 (s, 4H), 2.41 (s, 3H), 1.90 (s, 3H). | m/z calcd for C₂₁H₁₉F₄N₄O₂ [M + H]⁺: 435.1439; found: 435.1453 |
| 60 | | | |
| 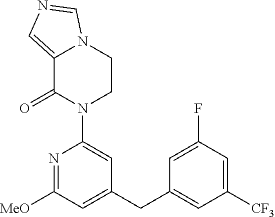 [SMILES: COc1cc(Cc2cc(F)cc(C(F)(F)F)c2)cc(N2CCn3cncc3C2=O)n1] | 130 °C, 48 h, 18% | (400 MHz, Acetone-d6) δ 7.77 (s, 1H), 7.65-7.60 (m, 2H), 7.56 (s, 1H), 7.45 (d, J = 9.5 Hz, 1H), 7.39 (d, J = 8.7 Hz, 1H), 6.52 (s, 1H), 4.58-4.52 (m, 2H), 4.50-4.47 (m, 2H), 4.16 (s, 2H), 3.89 (s, 3H). | m/z calcd for C₂₀H₁₇F₄N₄O₂ [M + H]⁺: 421.1282; found: 421.1299. |
| 61 | | | |
| 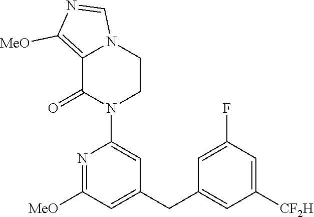 [SMILES: COc1cc(Cc2cc(F)cc(C(F)F)c2)cc(N2CCn3cnc(OC)c3C2=O)n1] | 130 °C, 36 h, 36% | (400 MHz, Acetone-d6) δ 7.62 (s, 1H), 7.43 (s, 1H), 7.41-7.37 (m, 1H), 7.33- 7.27 (m, 1H), 7.24-7.21 (m, 1H), 6.91 (t, J = 55.9 Hz, 1H), 6.46-6.41 (m, 1H), 4.50-4.45 (m, 2H), 4.40- 4.35 (m, 2H), 4.08 (s, 2H), 3.94 (s, 3H), 3.87 (s, 3H). | m/z calcd for C₂₁H₂₀F₃N₄O₃ [M + H]⁺: 433.1482; found: 433.1488, and m/z calcd for C₂₁H₁₉F₃N₄O₃Na [M + Na]⁺: 455.1301; found: 455.1306. |
| 62 | | | |
| 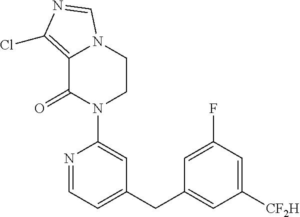 [SMILES: O=C1c2c(Cl)ncn2CCN1c1cc(Cc2cc(F)cc(C(F)F)c2)ccn1] | 110 °C, 16 h, 21% | (400 MHz, Acetone-d6) δ 8.34 (d, J = 5.1 Hz, 1H), 7.98-7.97 (m, 1H), 7.76 (s, 1H), 7.41 (s, 1H), 7.32-7.30 (m, 1H), 7.26-7.20 (m, 1H), 7.10 (dd, J = 5.1, 1.5 Hz, 1H), 6.91 (t, J = 55.9 Hz, 1H), 4.55-4.49 (m, 4H), 4.19 (s, 2H). | m/z calcd for C₁₉H₁₅ClF₃N₄O [M + H]⁺: 407.0881; found: 407.0883. |
| 63 | | | |
| 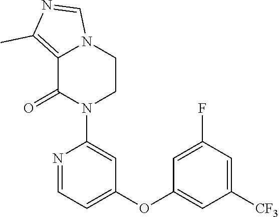 [SMILES: Cc1ncn2c1C(=O)N(c1cc(Oc3cc(F)cc(C(F)(F)F)c3)ccn1)CC2] | 130 °C, 48 h, 52% | (400 MHz, Acetone-d6) δ 8.39 (d, J = 5.7 Hz, 1H), 7.81 (d, J = 2.3 Hz, 1H), 7.64 (s, 1H), 7.47-7.43 (m, 1H), 7.42 (s, 1H), 7.36 (dt, J = 9.5, 2.3 Hz, 1H), 6.88 (dd, J = 5.7, 2.3 Hz, 1H), 4.54- 4.48 (m, 2H), 4.45-4.41 (m, 2H), 2.42 (s, 3H). | m/z calcd for C₁₉H₁₅F₄N₄O₂ [M + H]⁺: 407.1126; found: 407.1127 |
| 64 | | | |
| 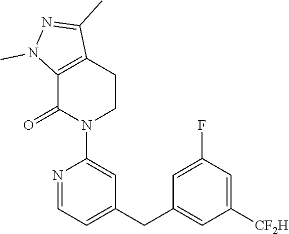 [SMILES: Cc1nn(C)c2c1CCN(c1cc(Cc3cc(F)cc(C(F)F)c3)ccn1)C2=O] | 110 °C, 16 h, 38% | (400 MHz, Acetone-d6) δ 8.34 (d, J = 5.1 Hz, 1H), 7.81 (s, 1H), 7.40 (s, 1H), 7.33-7.26 (m, 1H), 7.25-7.22 (m, 1H), 7.08 (dd, J = 5.1, 1.5 Hz, 1H), 6.90 (t, J = 55.9 Hz, 1H), 4.29 (t, J = 6.5 Hz, 2H), 4.17 (s, 2H), 4.05 (s, 3H), 2.82 (t, J = 6.5 Hz, 2H), 2.18 (s, 3H). | m/z calcd for C₂₁H₂₀F₃N₄O [M + H]⁺: 401.1584; found: 401.1587. |
| 65 | | | |
| 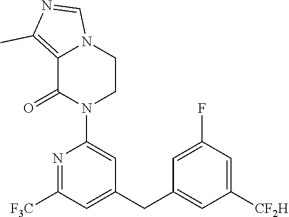 [SMILES: Cc1ncn2c1C(=O)N(c1cc(Cc3cc(F)cc(C(F)F)c3)cc(C(F)(F)F)n1)CC2] | 130 °C, 36 h, 47% | (400 MHz, Acetone-d6) δ 8.27 (s, 1H), 7.68 (s, 1H), 7.56 (d, J = 1.3 Hz, 1H), 7.46 (s, 1H), 7.37 (d, J = 9.6 Hz, 1H), 7.29-7.23 (m, 1H), 6.91 (t, J = 55.9 Hz, 1H), 4.53-4.43 (m, 4H), 4.33 (s, 2H), 2.46 (s, 3H). | m/z calcd for C₂₁H₁₇F₆N₄O [M + H]⁺: 455.1301; found: 455.1306. |
| 66 | | | |
| 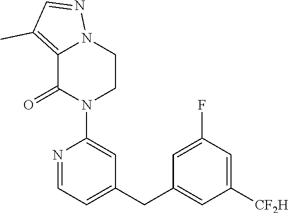 [SMILES: Cc1cnn2c1C(=O)N(c1cc(Cc3cc(F)cc(C(F)F)c3)ccn1)CC2] | 110 °C, 16 h, 66% | (400 MHz, Acetone-d6) δ 8.36 (d, J = 5.1 Hz, 1H), 7.98 (s, 1H), 7.41 (s, 1H), 7.38 (s, 1H), 7.31 (d, J = 9.6 Hz, 1H), 7.24 (d, J = 9.0 Hz, 1H), 7.11 (dd, J = 5.1, 1.5 Hz, 1H), 6.91 (t, J = 55.9 Hz, 1H), 4.56-4.53 (m, 2H), 4.50-4.43 (m, 2H), 4.19 (s, 2H), 2.32 (s, 3H). | m/z calcd for C₂₀H₁₈F₃N₄O [M + H]⁺: 387.1427; found: 387.1429. |
| 67 | | | |
| 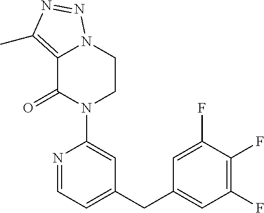 [SMILES: Cc1nnn2c1C(=O)N(c1cc(Cc3cc(F)c(F)c(F)c3)ccn1)CC2] | 130 °C, 48 h, 59% | (400 MHz, Acetone-d6) δ 8.38 (d, J = 5.1 Hz, 1H), 7.94-7.93 (m, 1H), 7.22- 7.12 (m, 3H), 4.81-4.76 (m, 2H), 4.68-4.63 (m, 2H), 4.12 (s, 2H), 2.51 (s, 3H). | m/z calcd for C₁₈H₁₅F₃N₅O [M + H]⁺: 374.1223; found: 374.1225. |
| 68 | | | |
| 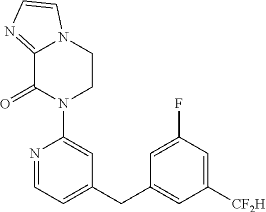 [SMILES: O=C1c2nccn2CCN1c1cc(Cc2cc(F)cc(C(F)F)c2)ccn1] | 110 °C, 24 h, 39% | (400 MHz, DMSO-d6) δ 8.37 (d, J = 5.1 Hz, 1H), 7.80 (s, 1H), 7.47 (s, 1H), 7.36 (d, J = 6.4 Hz, 2H), 7.30 (d, J = 9.0 Hz, 1H), 7.21 (s, 1H), 7.15-6.88 (m, 2H), 4.42-4.33 (m, 4H), 4.11 (s, 2H). | m/z calcd for C₁₉H₁₆F₃N₄O [M + H]⁺: 373.1272; found: 373.1271 |
| 69 | | | |

### Example 14. Synthesis of 7-(4-(3-(difluoromethyl)-5-fluorobenzyl)pyridin-2-yl)-6,7-dihydroimidazo[1,5-a]pyrazin-8(5H)-one (Compound 70)

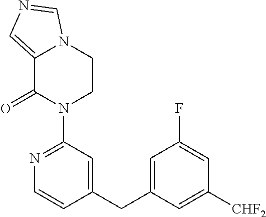 [SMILES: O=C1c2cncn2CCN1c1cc(Cc2cc(F)cc(C(F)F)c2)ccn1]

To a mixture of 6,7-dihydroimidazo[1,5-a]pyrazin-8(5H)-one (100 mg, 0.73 mmol), 2-bromo-4-(3-(difluoromethyl)-5-fluorobenzyl)pyridine (230 mg, 0.73 mmol), CuI (28 mg, 0.15 mmol) and K₂CO₃ (403 mg, 2.92 mmol) under nitrogen was added 1,4-dioxane (10 mL) followed by N,N′-dimethyl ethylenediamine (32 μL, 0.3 mmol). The reaction mixture was stirred at 110° C. for 16 h. Upon completion, the reaction was cooled to rt, diluted with EtOAc (50 mL), filtered, washed with EtOAc, and concentrated in vacuo. The residue was purified by automated reverse phase column chromatography eluting with MeCN:H₂O (5% to 100%) using Biotage Selekt® unit to afford the title compound as a colorless solid (172 mg, 63%). Recrystallisation: 80 mg was weighed into a 20 mL vial. 10 mL of 40% MeOH in H₂O was added then the mixture warmed in the water bath (80-90° C.) until all solid was dissolved and a clear solution obtained. After that, the vial was allowed to cool slowly and kept at RT overnight. Upon completion, the mixture was filtered and washed with a cool solution of MeOH:H₂O (1:5) (3 times), then dried in vacuo to obtain 50.07 mg of the title compound as a colourless crystalline solid. HPLC 254 nm (purity)>98%, melting point measured by microscope: 119-120° C. ¹H NMR (400 MHz, DMSO-d6) δ 8.36 (d, J=4.7 Hz, 1H), 7.94 (s, 1H), 7.80 (s, 1H), 7.65 (s, 1H), 7.38-7.35 (m, 2H), 7.30 (d, J=9.0 Hz, 1H), 7.16-6.87 (m, 2H), 4.41-4.29 (m, 4H), 4.10 (s, 2H). LC/MS (APCI): m/z=373.1 [M+H]⁺, Rt 3.179 min.

### Example 15. Synthesis of 7-(4-(3-(difluoromethyl)-5-fluorobenzyl)pyridin-2-yl)-1-methyl-6,7-dihydroimidazo[1,5-a]pyrazin-8(5H)-one (Compound 71)

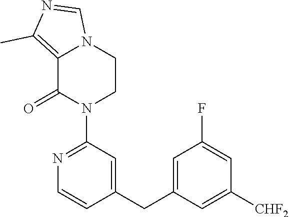 [SMILES: Cc1ncn2c1C(=O)N(c1cc(Cc3cc(F)cc(C(F)F)c3)ccn1)CC2]

To a mixture of 1-methyl-6,7-dihydroimidazo[1,5-a]pyrazin-8(5H)-one (150 mg, 0.99 mmol), 2-bromo-4-(3-(difluoromethyl)-5-fluorobenzyl)pyridine (314 mg, 0.99 mmol), CuI (38 mg, 0.20 mmol) and K₂CO₃ (549 mg, 3.97 mmol) under nitrogen was added 1,4-dioxane (15 mL) followed by N,N′-dimethyl ethylenediamine (43 μL, 0.04 mmol). The reaction mixture was stirred at 110° C. for 24 h. Upon completion, the reaction was cooled to rt, diluted with EtOAc (100 mL), filtered, washed with EtOAc, and concentrated in vacuo. The residue was purified by automated reverse phase column chromatography eluting with MeCN:H₂O (5% to 100%) using Biotage Selekt® unit to afford a colorless solid (118 mg, 31%). The solid was added into a 25 ml conical flask, then a mixture of MeOH (7 mL) and water (10 mL) was added into the flask. The mixture was warmed in the water bath at 80° C. until all solid was dissolved and a clear solution was obtained. The flash was kept at room temperature (no disturbance) overnight. Upon completion, the crystals were filtered, washed with water, and dried in vacuo for 8 h to give the title compound (87 mg) as a crystalline solid. HPLC 254 nm (purity)>98%, ¹H NMR (400 MHz, Acetone-d6) δ 8.31 (dd, J=5.1, 0.8 Hz, 1H), 7.95 (dt, J=1.4, 0.7 Hz, 1H), 7.61 (s, 1H), 7.39 (s, 1H), 7.28 (d, J=9.6 Hz, 1H), 7.22 (d, J=9.0 Hz, 1H), 7.05 (dd, J=5.1, 1.5 Hz, 1H), 6.89 (t, J=55.9 Hz, 1H), 4.46-4.35 (m, 4H), 4.15 (s, 2H), 2.43 (s, 3H), LC/MS (APCI): m/z=387.0 [M+H]⁺, Rt 3.187 min, melting point measured under microscope: 118.2-119.8° C.

### Example 16. Synthesis of 7-(3-(3-fluoro-5-(trifluoromethyl)benzyl)phenyl)-1-methyl-6,7-dihydroimidazo[1,5-a]pyrazin-8(5H)-one (Compound 72)

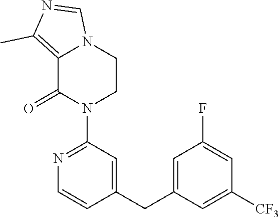 [SMILES: Cc1ncn2c1C(=O)N(c1cc(Cc3cc(F)cc(C(F)(F)F)c3)ccn1)CC2]

To a mixture of 1-methyl-6,7-dihydroimidazo[1,5-a]pyrazin-8(5H)-one (300 mg, 1.98 mmol), 2-chloro-4-(3-fluoro-5-(trifluoromethyl)benzyl)pyridine (575 mg, 1.98 mmol), CuI (76 mg, 0.40 mmol) and K₂CO₃ (1.10 mg, 7.94 mmol) under nitrogen was added 1,4-dioxane (20 mL) followed by N,N′-dimethyl ethylenediamine (86 μL, 0.80 mmol). The reaction mixture was stirred at 125° C. for 36 h. Upon completion, the reaction was cooled to rt, diluted with EtOAc (100 mL), filtered, washed with EtOAc, and concentrated in vacuo. The residue was purified by automated reverse phase column chromatography eluting with MeCN:H₂O (5% to 100%) using Biotage Selekt® unit to afford an amorphous colorless solid (178 mg, 22%). The solid was added into a 25 mL conical flash, then 20 mL mixture of MeOH (9 mL) and water (11 mL) was added into the flash. The mixture was warmed in the water bath at 80° C. until all solid was dissolved and a clear solution was obtained. The flash was kept at room temperature (no disturbance) overnight. Upon completion, the crystals were filtered, washed with water, and dried in vacuo for 8 h to give the title compound (134 mg) as a colorless crystalline solid. HPLC 254 nm (purity)>98%, ¹H NMR (400 MHz, Acetone-d6) δ 8.32 (dd, J=5.1, 0.8 Hz, 1H), 7.96 (dd, J=1.6, 0.8 Hz, 1H), 7.61 (s, 1H), 7.56 (s, 1H), 7.46-7.40 (m, 1H), 7.40-7.34 (m, 1H), 7.07 (dd, J=5.1, 1.5 Hz, 1H), 4.47-4.34 (m, 4H), 4.21 (s, 2H), 2.43 (s, 3H). LC/MS (APCI): m/z=405.0 [M+H]⁺, Rt 3.355 min. Melting point measured under microscope: 151.1-151.9° C.

### Example 17. Synthesis of mixture of 5-(4-(3-(difluoromethyl)-5-fluorobenzyl)pyridin-2-yl)-2-methyl-2,5,6,7-tetrahydro-4H-pyrazolo[4,3-c]pyridin-4-one (Compound 73a) and 5-(4-(3-(difluoromethyl)-5-fluorobenzyl)pyridin-2-yl)-1-methyl-1,5,6,7-tetrahydro-4H-pyrazolo[4,3-c]pyridin-4-one (Compound 73b)

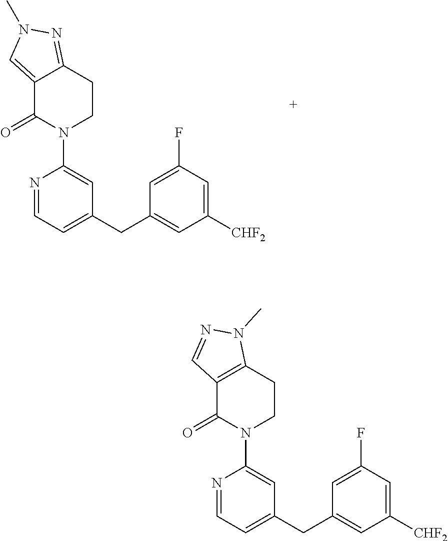 [SMILES: Cn1cc2c(n1)CCN(c1cc(Cc3cc(F)cc(C(F)F)c3)ccn1)C2=O ; Cn1ncc2c1CCN(c1cc(Cc3cc(F)cc(C(F)F)c3)ccn1)C2=O]

Step 1: A mixture of piperidine-2,4-dione (500 mg, 4.42 mmol) and DMA-DMF (6 mL) was stirred at 100° C. for 3 h. Upon completion, the mixture was added to water (20 mL) and extracted with EtOAc (3×50 mL). The combined organic layer was washed with brine, dried over MgSO₄ and concentrated in vacuo. The crude dark red oil 3-((dimethylamino)methylene) piperidine-2,4-dione (325 mg, 1.93 mmol) was dissolved in 2-methoxyethanol (4 mL) followed by adding hydrazine monohydrate (188 μL, 3.86 mmol), and the mixture was stirred at 130° C. for 12 h. Upon completion, water was added (20 mL) and the mixture extracted with EtOAc (3×50 mL). The combined organic layer was washed with brine, dried over MgSO₄ and concentrated in vacuo. The residue was purified by flash silica gel column chromatography eluting with MeOH:EtOAc (1:9) to afford 1,5,6,7-tetrahydro-4H-pyrazolo[4,3-c]pyridin-4-one as a colorless solid (112 mg, 18%). LC/MS (APCI): m/z=138.1 [M+H]⁺, Rt 0.51 min. ¹H NMR (400 MHz, DMSO-d6) δ 13.02 (s, 1H), 8.03 (br s, 1H), 7.28 (s, 1H), 3.40-3.36 (m, 2H), 2.79 (s, 2H).

Step 2: To a solution of 1,5,6,7-tetrahydro-4H-pyrazolo[4,3-c]pyridin-4-one (150 mg, 1.09 mmol) in DMF (1 mL) was added iodomethane (72 μL, 1.15 mmol) and the mixture was stirred at 60° C. for 24 h. Upon completion, the mixture was directly purified by flash column chromatography eluting with MeOH:EtOAc (1:9) to afford a yellow oil (45 mg, 27%, which was a mixture of 2 regioisomers 1-methyl-1,5,6,7-tetrahydro-4H-pyrazolo[4,3-c]pyridin-4-one and 2-methyl-2,5,6,7-tetrahydro-4/I-pyrazolo[4,3-c]pyridin-4-one (ratio 1.7:1, based on ¹H NMR). LC/MS (APCI): m/z=152.1 [M+H]⁺, Rt 0.533 min. ¹H NMR (400 MHz, DMSO-d6) (mixture of 2 regioisomers) δ 8.00 (s, 1H, major), 7.63 (s, 1H, minor), 7.29 (s, 1H, major), 7.19 (s, 1H, minor), 3.81 (s, 3H, major), 3.75 (s, 3H, minor), 2.87 (t, J=6.9 Hz, 2H, major & minor), 2.71 (t, J=6.7 Hz, 2H, major & minor).

Step 3: To a mixture of the 2 regioisomers from step 2 (40 mg, 0.26 mmol), 2-bromo-4-(3-(difluoromethyl)-5-fluorobenzyl)pyridine (84 mg, 0.26 mmol), CuI (10 mg, 0.053 mmol) and K₂CO₃ (146 mg, 1.06 mmol) under nitrogen was added 1,4-dioxane (4 mL) followed by DMEDA (6 μL, 0.053 mmol). The reaction mixture was stirred at 110° C. for 16 h. Upon completion, the reaction was cooled to RT, diluted with EtOAc (10 mL), filtered, washed with EtOAc, and concentrated in vacuo. The residue was purified by automated reverse phase column chromatography eluting with MeCN:H₂O (5% to 100%) using Biotage Selekt® unit to afford the title compounds as a colorless solid (mixture of 2 regioisomers, 26 mg, 25%). Further purification by flash silica gel column chromatography eluting with EtOAc:PET (1:1) isolated 3 fractions which include fraction 1 (5-(4-(3-(difluoromethyl)-5-fluorobenzyl)pyridin-2-yl)-2-methyl-2,5,6,7-tetrahydro-4H-pyrazolo[4,3-c]pyridin-4-one, 5 mg), fraction 2 (5-(4-(3-(difluoromethyl)-5-fluorobenzyl)pyridin-2-yl)-1-methyl-1,5,6,7-tetrahydro-4H-pyrazolo[4,3-c]pyridin-4-one, 0.5 mg) and fraction 3 (mixture, 1:1 ratio, 14 mg).

5-(4-(3-(difluoromethyl)-5-fluorobenzyl)pyridin-2-yl)-2-methyl-2,5,6,7-tetrahydro-4H-pyrazolo[4,3-c]pyridin-4-one: ¹H NMR (400 MHz, Acetone-d6) δ 8.36 (s, 1H), 8.07 (s, 1H), 7.85 (s, 1H), 7.39 (s, 1H), 7.29 (d, J=9.7 Hz, 1H), 7.23 (d, J=9.0 Hz, 1H), 7.08-6.75 (m, 2H), 4.32 (t, J=6.4 Hz, 2H), 4.13 (s, 2H), 3.93 (s, 3H), 2.95 (t, J=6.4 Hz, 2H). LC/MS (APCI): m/z=387.0 [M+H]⁺, Rt 3.50 min.

5-(4-(3-(difluoromethyl)-5-fluorobenzyl)pyridin-2-yl)-1-methyl-1,5,6,7-tetrahydro-4H-pyrazolo[4,3-c]pyridin-4-one: ¹H NMR (400 MHz, Acetone-d6) δ 8.34 (s, 1H), 7.83 (s, 1H), 7.76 (s, 1H), 7.39 (s, 1H), 7.29 (d, J=9.6 Hz, 1H), 7.23 (d, J=8.7 Hz, 1H), 7.07-6.74 (m, 2H), 4.37 (t, J=6.5 Hz, 2H), 4.13 (s, 2H), 3.85 (d, J=3.8 Hz, 3H), 3.12 (t, J=6.5 Hz, 2H). LC/MS (APCI): m/z=445.1 [M+H]⁺, Rt 3.51 min.

### Example 18. Synthesis of 5-(4-(3-(difluoromethyl)-5-fluorobenzyl)pyridin-2-yl)-1,5,6,7-tetrahydro-4H-pyrazolo[4,3-c]pyridin-4-one (Compound 74)

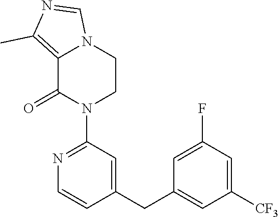 [SMILES: Cc1ncn2c1C(=O)N(c1cc(Cc3cc(F)cc(C(F)(F)F)c3)ccn1)CC2]

Step 1: To a solution of 1,5,6,7-tetrahydro-4H-pyrazolo[4,3-c]pyridin-4-one (100 mg, 0.73 mmol) in CH₂Cl₂ (5 mL) at 0° C. was added DIPEA (381 μL, 2.19 mmol) and SEMCl (142 μL, 0.8 mmol), and the mixture was stirred at rt for 16 h. Upon completion, water (50 mL) was added, and the mixture was extracted with EtOAc (3×50 mL). The combined organic layer was washed with brine, dried over MgSO₄ and concentrated in vacuo. The residue was purified by flash column chromatography eluting with EtOAc (100%) to afford a pale-yellow solid (112 mg, 57%, containing an inseparable mixture of the 2 regioisomers of SEM-protected adduct, ratio 2:1). The mixture was used for the next step without further purification. LC/MS (APCI): m/z=268.1 [M+H]⁺, Rt 3.327 min. ¹H NMR (400 MHz, CDCl₃) (mixture of 2 regioisomers, ratio 2:1) δ 7.98 (s, 0.5H, minor), 7.90 (s, 1H, major), 5.60 (br s, 0.5H, minor), 5.50 (br s, 1H, major), 5.43 (s, 2H, major), 5.40 (s, 1H, minor), 3.66-3.55 (m, 6H, 4H major+2H minor), 3.01 (t, J=6.9 Hz, 2H, major), 2.95 (t, J=6.7 Hz, 1H, minor), 0.96-0.85 (m, 3H, 2H major+1H minor), −0.01 (s, 4H, minor), −0.02 (s, 9H, major).

Step 2: To a mixture of the crude material from step 1 (40 mg, 0.15 mmol), 2-bromo-4-(3-(difluoromethyl)-5-fluorobenzyl)pyridine (47 mg, 0.15 mmol), CuI (5.7 mg, 0.03 mmol) and K₂CO₃ (83 mg, 0.60 mmol) under nitrogen was added 1,4-dioxane (4 mL) followed by DMEDA (6.5 μL, 0.06 mmol). The reaction mixture was stirred at 110° C. for 24 h. Upon completion, the reaction was cooled to rt, diluted with EtOAc (50 mL), filtered, washed with EtOAc, and concentrated in vacuo. The residue was purified by automated reverse phase column chromatography eluting with MeCN:H₂O (5% to 100%) using Biotage Selekt® unit to afford a colorless oil (a mixture of 2 regioisomers: 5-(4-(3-(difluoromethyl)-5-fluorobenzyl)pyridin-2-yl)-1-((2-(trimethylsilyl) ethoxy)methyl)-1,5,6,7-tetrahydro-4H-pyrazolo[4,3-c]pyridin-4-one and 5-(4-(3-(difluoromethyl)-5-fluorobenzyl)pyridin-2-yl)-2-((2-(trimethylsilyl) ethoxy)methyl)-2,5,6,7-tetrahydro-4H-pyrazolo[4,3-c]pyridin-4-one 32 mg, 43%). LC/MS (APCI): m/z=503.1 [M+H]⁺, Rt 4.20 min.

Step 3: To a solution of the crude mixture from step 2 (30 mg, 0.06 mmol) in EtOH (0.5 mL) was added slowly concentrated HCl (0.5 mL) and the mixture was stirred at 80° C. for 16 h. Upon completion, the solvent was removed in vacuo, the residue was poured into a saturated NaHCO₃ solution (20 mL) and extracted with EtOAc (3×50 mL). The combined organic layer was washed with brine, dried over MgSO₄, and concentrated in vacuo. The residue was purified by automated reverse phase column chromatography eluting with MeCN:H₂O (5% to 100%) using Biotage Selekt® unit to afford 5-(4-(3-(difluoromethyl)-5-fluorobenzyl)pyridin-2-yl)-1,5,6,7-tetrahydro-4H-pyrazolo[4,3-c]pyridin-4-one as a colorless solid (6 mg, 27%). HPLC 254 nm (purity)>98%, LC/MS (APCI): m/z=373.0 [M+H]⁺, Rt 3.356 min, HRMS (ESI) m/z calcd for C19H₁₆F₃N₄O [M+H]⁺: 373.1271; found: 373.1278. ¹H NMR (400 MHz, DMSO-d6) δ 13.21 (br s, 1H), 8.33 (d, J=5.1 Hz, 1H), 8.27 (s, 1H), 7.65 (s, 1H), 7.35 (d, J=5.7 Hz, 2H), 7.29 (d, J=9.0 Hz, 1H), 7.16-6.86 (m, 2H), 4.21 (t, J=6.4 Hz, 2H), 4.08 (s, 2H), 2.96 (br s, 2H).

### Example 19. Synthesis of 3-fluoro-5-((2-(3-methyl-5,6-dihydroimidazo[1,5-a]pyrazin-7 (8H)-yl)pyridin-4-yl)methyl)benzonitrile (Compound 75)

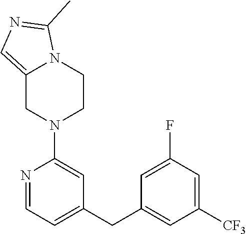 [SMILES: Cc1ncc2n1CCN(c1cc(Cc3cc(F)cc(C(F)(F)F)c3)ccn1)C2]

To a sealed tube was added 3-((2-chloropyridin-4-yl)methyl)-5-fluorobenzonitrile (50 mg, 0.20 mmol), 2-methyl-5,6,7,8-tetrahydroimidazo[1,5-a]pyrazine (39 mg, 0.28 mmol), Pd₂(dba)₂ (19 mg, 0.02 mmol), RuPhos (19 mg, 0.04 mmol), t-BuONa (39 mg, 0.41 mmol) and 1,4-dioxane (3 mL). The mixture was stirred at 100° C. for 3 h. Upon completion, the mixture was diluted by EtOAc and filtered through a pad of Celite®. The filtrate was concentrated in vacuo, and the residue was purified by automated column eluting with MeCN (+1% TFA): H₂O (+1% TFA) (5% to 100%) using Biotage Selekt® unit to afford the title compound as a TFA salt. After column chromatography, the product was neutralized by saturated aqueous NaHCO₃ solution (10 mL) and extracted with CH₂Cl₂ (3× 10 mL). The combined organic layer was dried over MgSO₄ and concentrated to obtain the title compound as a yellow oil. ¹H NMR (400 MHz, Acetone-d6) δ 8.14 (d, J=5.2 Hz, 1H), 7.57 (s, 1H), 7.52-7.45 (m, 2H), 7.37 (s, 1H), 7.08 (s, 1H), 6.74 (d, J=5.2 Hz, 1H), 4.97 (s, 2H), 4.38 (t, J=5.5 Hz, 2H), 4.26 (t, J=5.5 Hz, 2H), 4.08 (s, 2H), 2.72 (s, 3H), HRMS (ESI) m/z calcd for C₂₀H₁₉FN₅ [M+H]⁺: 348.1619; found: 348.1611.

### Example 20. Synthesis of 7-(4-(3-(difluoromethyl)-5-fluorobenzyl)pyridin-2-yl)-3-methyl-5,6,7,8-tetrahydroimidazo[1,5-a]pyrazine (Compound 76)

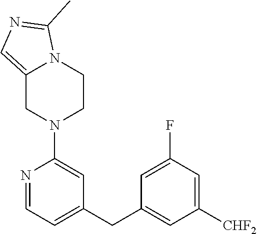 [SMILES: Cc1ncc2n1CCN(c1cc(Cc3cc(F)cc(C(F)F)c3)ccn1)C2]

To a sealed tube was added 2-bromo-4-(3-(difluoromethyl)-5-fluorobenzyl)pyridine (60 mg, 0.19 mmol), 2-methyl-5,6,7,8-tetrahydroimidazo[1,5-a]pyrazine (37 mg, 0.27 mmol), Pd₂(dba)₃ (17 mg, 0.19 mmol), RuPhos (18 mg, 0.38 mmol), t-BuONa (37 mg, 0.38 mmol) and 1,4-dioxane (3 mL). The mixture was stirred at 100° C. for 3 h. Upon completion, the mixture was diluted by EtOAc and filtered through a pad of Celite®. The filtrate was concentrated in vacuo, and the residue was purified by automated column eluting with MeCN (+1% TFA): H₂O (+1% TFA) (5% to 100%) using Biotage Selekt® unit to afford the title compound as a TFA salt. After column chromatography, the TFA salt product was neutralised by saturated aqueous NaHCO₃ solution (10 mL) and extracted with CH₂Cl₂ (3×10 mL). The combined organic layer was dried over MgSO₄ and concentrated to obtain the title compound as a yellow oil (32 mg, 45%). ¹H NMR (400 MHz, Acetone-d6) δ 8.16 (d, J=5.3 Hz, 1H), 7.43 (s, 1H), 7.37 (s, 1H), 7.29-7.20 (m, 2H), 7.13 (s, 1H), 7.03-6.74 (m, 2H), 5.01 (s, 2H), 4.42 (t, J=5.5 Hz, 2H), 4.29 (t, J=5.5 Hz, 2H), 4.09 (s, 2H), 2.74 (s, 3H). HRMS (ESI) m/z calcd for C20H₂₀F₃N4 [M+H]⁺: 373.1635; found: 373.1630.

### Example 21. Synthesis of 7-(3-(3-fluoro-5-(trifluoromethyl)benzyl)phenyl)-6,7-dihydroimidazo[1,5-a]pyrazin-8(5H)-one (Compound 77)

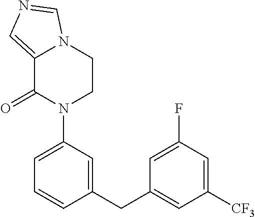 [SMILES: O=C1c2cncn2CCN1c1cccc(Cc2cc(F)cc(C(F)(F)F)c2)c1]

Step 1: K₂CO₃ (1.08 g, 7.78 mmol) was added to a stirred solution of methyl 3-fluoro-5-(trifluoromethyl)benzyl bromide (500 mg, 1.95 mmol) and tert-butyl (3-(4,4,5,5-tetramethyl-1,3,2-dioxaborolan-2-yl)phenyl)carbamate (559 mg, 1.75 mmol) in 1,4-dioxane (7 mL) and water (3 mL). The reaction was degassed with N₂ for 20 min then Pd(dppf)₂Cl₂·CH₂Cl₂ complex (160 mg, 0.20 mmol) was added. The reaction mixture was heated at 100° C. for 2 h. The reaction mixture was partitioned between water (50 mL) and EtOAc (50 mL). The organic layer was separated, and the water layer was extracted with EtOAc (2× 50 mL). The combined organic layer was dried over MgSO₄ and concentrated in vacuo. The residue was purified by flash column chromatography eluting with EtOAc:PET ether (1:10) to afford tert-butyl (3-(3-fluoro-5-(trifluoromethyl)benzyl)phenyl)carbamate as a colorless solid (492 mg, 68%). ¹H NMR (400 MHz, Acetone-d6) δ 8.35 (s, 1H), 7.51-7.45 (m, 2H), 7.43 (d, J=8.3 Hz, 1H), 7.37-7.30 (m, 2H), 7.23 (t, J=7.8 Hz, 1H), 6.92 (d, J=7.6 Hz, 1H), 4.10 (s, 2H), 1.47 (s, 9H).

Step 2: To a solution of tert-butyl (3-(3-fluoro-5-(trifluoromethyl)benzyl)phenyl)carbamate (490 mg, 1.33 mmol) in anhydrous DMF (8 mL) at 0° C. was added NaH (60% in mineral oil, 106 mg, 2.65 mmol) and the mixture was stirred at 0° C. for 15 min. After that, a solution of (2-bromoethoxy) (tert-butyl)dimethylsilane (370 μL, 1.72 mmol) in 0.5 mL anhydrous DMF was added dropwise, and the mixture was stirred at rt for 16 h. Upon completion, the mixture was cooled to 0° C., quenched by adding slowly water (20 mL), and then extracted with EtOAc (3×50 mL). The combined organic layer was washed with brine, dried over MgSO₄, and concentrated in vacuo. The residue was purified by flash silica gel column chromatography eluting with 5% EtOAc in petroleum ether to afford the inseparable mixture of tert-butyl(2-((tert-butyldimethylsilyl)oxy)ethyl) (3-(3-fluoro-5-(trifluoromethyl)benzyl)phenyl)carbamate and unreacted tert-butyl (3-(3-fluoro-5-(trifluoromethyl)benzyl)phenyl)carbamate as colorless oil (255 mg), which was observed by ¹H NMR. The mixture was used for the next step without further purification.

Step 3: To a solution of tert-butyl(2-((tert-butyldimethylsilyl)oxy)ethyl) (3-(3-fluoro-5-(trifluoromethyl)benzyl)phenyl)carbamate (255 mg, 0.41 mmol calculated based on the drawn structure) in anhydrous THF (10 mL) at rt was added TBAF (1M in THF, 725 μL, 0.73 mmol) and the mixture was stirred for 6 h at rt. Upon completion, water (40 mL) was added, and the mixture was extracted with EtOAc (3×50 mL). The combined organic layer was washed with brine, dried over MgSO₄ and concentrated in vacuo. The residue was purified by flash column chromatography eluting with EtOAc:PET (1:1) to afford tert-butyl (3-(3-fluoro-5-(trifluoromethyl)benzyl)phenyl) (2-hydroxyethyl)carbamate as colorless oil (141 mg, 26% after 2 steps). ¹H NMR (400 MHz, Acetone-d6) δ 7.48 (s, 1H), 7.37-7.25 (m, 4H), 7.22-7.19 (m, 1H), 7.18-7.12 (m, 1H), 4.51-4.46 (m, 1H), 4.14 (s, 2H), 3.73-3.63 (m, 4H), 1.34 (s, 9H).

Step 4: To a solution of methyl 3H-imidazole-4-carboxylate (40 mg, 0.32 mmol) and tert-butyl (3-(3-fluoro-5-(trifluoromethyl)benzyl)phenyl) (2-hydroxyethyl)carbamate (138 mg, 0.33 mmol) in anhydrous THF (8 mL) at −40° C. was added Ph₃P (208 mg, 0.793 mmol). DIAD (156 μL, 0.79 mmol) was added dropwise into the solution and the reaction was stirred at −40° C. for 2 h then increased slowly to rt and stirred overnight. Upon completion, silica gel was added and the mixture was concentrated in vacuo. The mixture was loaded and purified by flash silica gel column chromatography eluting with EtOAc: petroleum ether (1:1) to afford methyl 1-(2-((tert-butoxycarbonyl) (3-(3-fluoro-5-(trifluoromethyl)benzyl)phenyl)amino)ethyl)-1H-imidazole-5-carboxylate as colorless oil (92 mg, 56%). ¹H NMR (400 MHz, Acetone-d6) δ 7.70 (s, 1H), 7.58 (s, 1H), 7.51 (s, 1H), 7.39-7.34 (m, 2H), 7.31-7.24 (m, 1H), 7.13 (d, J=7.7, 1H), 6.99-6.97 (m, 2H), 4.52 (t, J=5.7 Hz, 2H), 4.15-4.04 (m, 4H), 3.76 (s, 3H), 1.32 (s, 9H), LC/MS (APCI): m/z=522.1 [M+H]⁺, Rt 3.720 min.

Step 5: To a solution of methyl 1-(2-((tert-butoxycarbonyl) (3-(3-fluoro-5-(trifluoromethyl)benzyl)phenyl)amino)ethyl)-1H-imidazole-5-carboxylate (90 mg, 0.17 mmol) in CH₂Cl₂ (2.0 mL) at rt was added TFA (1.0 mL) and the mixture was stirred at rt for 2 h before being concentrated in vacuo. Saturated aqueous solution of NaHCO₃ (30 mL) was added into the residue, and the mixture was stirred for 15 min and then extracted with EtOAc (3× 50 mL). The combined organic layer was washed with brine, dried over MgSO₄ and concentrated in vacuo. The residue (65 mg) was dissolved in MeCN (10 mL) and transferred into a sealed tube. Cs₂CO₃ (169 mg, 0.52 mmol) was added into the mixture at rt, and the mixture was stirred at 70° C. for 16 h. Upon completion, the mixture was diluted with EtOAc (50 mL) and filtered to remove insoluble solid. The filtrate was concentrated in vacuo and the residue was purified by reverse phase column chromatography eluting with MeCN:H₂O (5% to 100%) to afford the title compound as a pale-yellow solid (19 mg, 28%). ¹H NMR (400 MHz, Acetone-d6) δ 7.74 (s, 1H), 7.54 (d, J=0.9 Hz, 1H), 7.52 (s, 1H), 7.42 (t, J=2.0 Hz, 1H), 7.40-7.28 (m, 4H), 7.20-7.18 (m, 1H), 4.55-4.48 (m, 2H), 4.23-4.18 (m, 2H), 4.17 (s, 2H). HRMS (ESI) m/z calcd for C20H₁₆F₄N₃O [M+H]⁺: 390.1224; found: 390.1223.

### Example 22. Synthesis of 7-(4-(3-(difluoromethyl)-5-fluorobenzyl)pyridin-2-yl)-8-methyl-5,6,7,8-tetrahydroimidazo[1,5-a]pyrazine (Compound 78)

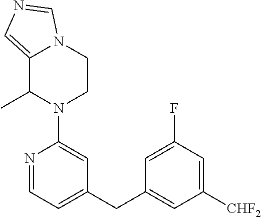 [SMILES: CC1c2cncn2CCN1c1cc(Cc2cc(F)cc(C(F)F)c2)ccn1]

Step 1: To a solution of 1-trityl-1H-imidazole-4-carbaldehyde (400 mg, 1.18 mmol) in anhydrous THF (10 mL) at 0° C. was added methyl magnesium bromide solution (3M in diethyl ether, 0.80 mL, 2.4 mmol) and the mixture was warmed to rt and stirred at rt for 2 h. Upon completion, the reaction was cooled to 0° C. and quenched by adding saturated aqueous ammonium chloride solution (50 mL) and the mixture was extracted with EtOAc (3× 100 mL). The combined organic layer was washed with brine, dried over MgSO₄ and concentrated in vacuo. The residue was purified by flash column chromatography eluting with EtOAc 100% to afford 1-(1-trityl-1H-imidazol-4-yl) ethan-1-ol as a colorless solid (385 mg, 92%). ¹H NMR (400 MHz, Acetone-d6) δ 7.40 (m, 9H), 7.27 (d, J=1.5 Hz, 1H), 7.20-7.13 (m, 6H), 6.78-6.73 (m, 1H), 4.73 (p, J=6.1 Hz, 1H), 3.83 (d, J=4.7 Hz, 1H), 1.38 (d, J=6.4 Hz, 3H).

Step 2: To a solution of 1-(1-trityl-1H-imidazol-4-yl) ethan-1-ol (2.00 g, 5.64 mmol) in DMF (30 mL) at rt was added imidazole (1.15 g, 16.9 mmol) then TBSCl (1.11 g, 7.34 mmol) and the mixture was stirred at rt for 5 h. Upon completion, water (200 mL) was added, and the mixture was extracted with EtOAc (3× 200 mL). The combined organic layer was washed with brine, dried over MgSO₄ and concentrated in vacuo. The residue was purified by flash column chromatography eluting with EtOAc:PET ether (1:2) to afford 4-(1-((tert-butyldimethylsilyl)oxy)ethyl)-1-trityl-1H-imidazole as colorless oil (2.11 g, 80%). ¹H NMR (400 MHz, Acetone-d6) δ 7.44-7.36 (m, 9H), 7.27 (d, J=1.5 Hz, 1H), 7.20-7.13 (m, 6H), 6.70-6.69 (m, 1H), 4.84 (dq, J=6.4, 0.9 Hz, 1H), 1.39 (d, J=6.4 Hz, 3H), 0.81 (s, 9H), 0.03 (s, 3H), −0.07 (s, 3H).

Step 3: To a solution of 4-(1-((tert-butyldimethylsilyl)oxy)ethyl)-1-trityl-1H-imidazole (300 mg, 0.64 mmol) in MeCN (5 mL) at room temperature was added bromoacetonitrile (295 μL, 4.22 mmol), and the mixture was stirred at 80° C. for 5 h. After that, the mixture was concentrated, and then the residue was dissolved into MeOH (10 mL) and refluxed at 80° C. for another 2 h. After concentration, the residue was dissolved into CH₂Cl₂ (100 mL). The solution was washed with saturated aqueous solution of NaHCO₃ (50 mL), brine (50 mL), and dried over MgSO₄. The residue was purified by flash silica gel column chromatography eluting with EtOAc 100% to afford 2-(5-(1-((tert-butyldimethylsilyl)oxy)ethyl)-1H-imidazol-1-yl) acetonitrile as a pale-yellow solid (64 mg, 38%). ¹H NMR (400 MHz, Acetone-d6) δ 7.69 (s, 1H), 6.87 (s, 1H), 5.46-5.31 (m, 2H), 5.23 (q, J=6.6 Hz, 1H), 1.58 (d, J=6.6 Hz, 3H), 0.90 (s, 9H), 0.11 (s, 3H), 0.06 (s, 3H).

Step 4: To a stirred solution of 2-(5-(1-((tert-butyldimethylsilyl)oxy)ethyl)-1H-imidazol-1-yl) acetonitrile (20 mg, 0.075 mmol) in anhydrous methanol (1 mL), cooled to 0° C., were added Boc₂O (33 mg, 0.15 mmol) and NiCl₂·6H₂O (2.0 mg, 0.08 mmol). NaBH₄ (20 mg, 0.53 mmol) was then added in small portions over 10 min. The reaction mixture turned black, was allowed to warm to rt and left to stir for a further 1 h. Upon completion, diethylenetriamine (16 μL, 0.15 mmol) was added. The mixture was allowed to stir for 30 min before solvent was removed in vacuo. The purple residue was dissolved in EtOAc (30 mL) and washed with saturated NaHCO₃ (2× 30 mL). The organic layer was dried over MgSO₄ and the solvent was removed in vacuo. The residue was purified by flash silica gel column chromatography eluting with EtOAc 100% to afford tert-butyl(2-(5-(1-((tert-butyldimethylsilyl)oxy)ethyl)-1H-imidazol-1-yl)ethyl)carbamate as colorless oil (16.1 mg, 57%). ¹H NMR (400 MHz, Acetone-d6) δ 7.46 (s, 1H), 6.82 (s, 1H), 6.30 (br s, 1H), 5.12 (q, J=6.5 Hz, 1H), 4.19 (t, J=6.5 Hz, 2H), 3.49-3.38 (m, 2H), 1.54 (d, J=6.5 Hz, 3H), 1.41 (s, 9H), 0.88 (s, 9H), 0.04 (d, J=5.7 Hz, 6H). LC/MS (APCI): m/z=370.2 [M+H]⁺, Rt 3.781 min (The desired product is non-UV active. Rt based on mass ion peak).

Step 5: To a solution of tert-butyl(2-(5-(1-((tert-butyldimethylsilyl)oxy)ethyl)-1H-imidazol-1-yl)ethyl)carbamate (150 mg, 0.41 mmol) in anhydrous THF (8 mL) at rt was added TBAF (1M in THF, 447 μL, 0.45 mmol) and the mixture was stirred for 5 h at rt. Upon completion, water (20 mL) was added, and the mixture was extracted with EtOAc (3×50 mL). The combined organic layer was washed with brine, dried over MgSO₄ and concentrated in vacuo to obtain tert-butyl(2-(5-(1-hydroxyethyl)-1H-imidazol-1-yl)ethyl)carbamate (pale yellow oil, 101 mg) which was used for the next step without further purification. LC/MS (APCI): m/z=256.1 [M+H]⁺, Rt 2.665 min (The desired product is non-UV active. Rt based on mass ion peak).

Step 6: To a solution of tert-butyl(2-(5-(1-hydroxyethyl)-1H-imidazol-1-yl)ethyl)carbamate (101 mg, 0.396 mmol) in 1,4-dioxane (10 mL) at rt was added MnO₂ (344 mg, 3.96 mmol) and the mixture was stirred at 100° C. for 6 h. After that, the mixture was filtered through a pad of Celite®, washed with EtOAc (50 mL) and the filtrate was concentrated in vacuo. The residue was purified by flash column chromatography eluting with 5% MeOH in EtOAc to afford tert-butyl(2-(5-acetyl-1H-imidazol-1-yl)ethyl)carbamate as a colorless solid (65 mg, 63% after 2 steps). ¹H NMR (400 MHz, Acetone-d6) δ 7.83 (s, 1H), 7.65 (s, 1H), 6.06 (br s, 1H), 4.41 (t, J=5.7 Hz, 2H), 3.40 (q, J=5.8 Hz, 2H), 2.43 (s, 3H), 1.36 (s, 9H). LC/MS (APCI): m/z=254.1 [M+H]⁺, Rt 2.843 min.

Step 7: To a solution of tert-butyl(2-(5-acetyl-1H-imidazol-1-yl)ethyl)carbamate (62 mg, 0.25 mmol) in CH₂Cl₂ (5 mL) at rt was added TFA (190 μL, 2.45 mmol) and the mixture was stirred at rt for 16 h. After that, the reaction was cooled to 0° C., then NaBH₄ (18 mg, 0.47 mmol) was added, and the mixture was stirred at rt for 30 min. Upon completion, the reaction was quenched by adding water (0.5 mL) and stirred for 5 min, then concentrated in vacuo to remove all solvents and obtain pale yellow oil (55 mg). The mixture was suspended in CH₂Cl₂ (20 mL) at rt and NaHCO₃ (400 mg, 5.0 mmol) was added. After stirring for 1 h at rt, the mixture was filtered, washed with CH₂Cl₂ (100 mL), and the filtrate was concentrated in vacuo to afford 8-methyl-5,6,7,8-tetrahydroimidazo[1,5-a]pyrazine as a pale-yellow oil (10.1 mg, 31%), which was used for the next step without further purification. LC/MS (APCI): m/z=138.2 [M+H]⁺, Rt 0.469 min (UV214 nm). ¹H NMR (400 MHz, Acetone-d6) δ 7.67 (s, 1H), 6.79 (s, 1H), 4.18-4.03 (m, 2H), 3.98 (m, 1H), 3.36 (ddd, J=13.3, 4.8, 2.3 Hz, 1H), 3.10 (ddd, J=13.2, 11.1, 4.5 Hz, 1H), 1.36 (d, J=6.6 Hz, 3H).

Step 8: To a sealed tube was added 2-bromo-4-(3-(difluoromethyl)-5-fluorobenzyl)pyridine (20 mg, 0.063 mmol), 8-methyl-5,6,7,8-tetrahydroimidazo[1,5-a]pyrazine (10 mg, 0.070 mmol), Pd₂(dba)₃ (6 mg, 0.006 mmol), Ruphos (6 mg, 0.01 mmol), t-BuONa (12 mg, 0.13 mmol) and 1,4-dioxane (2 mL). The mixture was stirred at 100° C. for 3 h. Upon completion, the mixture was diluted by EtOAc and filtered through a pad of Celite®. The filtrate was concentrated in vacuo, and the residue was purified by automated column eluting with MeCN (+0.1% TFA): H₂O (+0.1% TFA) (5% to 100%) using Biotage Selekt® unit to afford a colorless oil (9.1 mg, TFA salt). The TFA salt was neutralized by aqueous saturated NaHCO₃ solution (10 mL) and extracted with CH₂Cl₂ (3× 10 mL). The combined organic layer was dried over MgSO₄ and concentrated to obtain the title compound as colorless oil (6.5 mg, 28%). ¹H NMR (400 MHz, DMSO-d6) δ 9.02 (s, 1H), 8.06 (d, J=5.1 Hz, 1H), 7.62 (s, 1H), 7.38-7.35 (m, 2H), 7.28 (d, J=9.0 Hz, 1H), 7.16-6.85 (m, 2H), 6.61 (d, J=5.1 Hz, 1H), 5.86 (q, J=6.8 Hz, 1H), 4.62 (d, J=14.2 Hz, 1H), 4.43 (d, J=12.6 Hz, 1H), 4.17-4.07 (m, 2H), 3.97 (s, 2H), 1.45 (d, J=6.7 Hz, 3H). HRMS (ESI) m/z calcd for C₂₀H₂₀F₃N₄ [M+H]⁺: 373.1635; found: 373.1636. HPLC 254 nm (purity)>98%.

### Example 23. Synthesis of 3-(difluoromethyl)-7-(4-(3-(difluoromethyl)-5-fluorobenzyl)pyridin-2-yl)-6,7-dihydroimidazo[1,5-a]pyrazin-8(5H)-one (Compound 79)

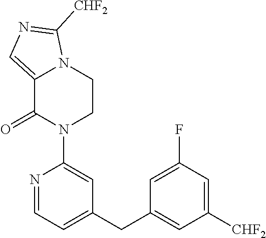 [SMILES: O=C1c2cnc(C(F)F)n2CCN1c1cc(Cc2cc(F)cc(C(F)F)c2)ccn1]

TFA (10 μL) was added to a solution of 7-(4-(3-(difluoromethyl)-5-fluorobenzyl)pyridin-2-yl)-6,7-dihydroimidazo[1,5-a]pyrazin-8(5H)-one (30 mg, 0.081 mmol) and DFMS (74 mg) in DMSO (0.7 mL) in a small screw-capped vial. The reaction was cooled in ice as TBHP (5.0-6.0 M in decane) (52 μL) was added slowly to the stirred solution. The vial was sealed and stirred at 50° C. for 4 h. The reaction mixture was cooled to RT, transferred to a separating funnel (rinsing with EtOAc), and diluted with 10 mL sodium bicarbonate solution. The aqueous solution was extracted into EtOAc (2×20 mL), and the combined organic phases were washed with brine, dried (MgSO₄), and concentrated in vacuo. The residue was purified by flash silica gel column chromatography (100% EtOAc) to obtain 2 fractions. Fraction 1:5 mg (eluting first) and fraction 2:2 mg (eluting second). Further purification of fraction 1 by reverse column chromatography (eluting with MeCN:H₂O, 5% to 100%) gave the title compound (2.1 mg, colorless oil). ¹H NMR (400 MHz, Acetone-d6) δ 8.37 (d, J=5.1 Hz, 1H), 8.00 (s, 1H), 7.71 (s, 1H), 7.40 (s, 1H), 7.30 (d, J=9.7 Hz, 1H), 7.24 (d, J=9.0 Hz, 1H), 7.20-6.76 (m, 3H), 4.66-4.56 (m, 4H), 4.19 (s, 2H). HRMS (ESI) m/z calcd for C20H₁₆F₅N₄O [M+H]⁺: 423.1239; found: 423.1240.

### Example ₂₄. Synthesis of 7-(4-(3-(difluoromethyl)-5-fluorobenzyl)pyridin-2-yl)-5,6,7,8-tetrahydroimidazo[1,5-a]pyrazine (Compound 80)

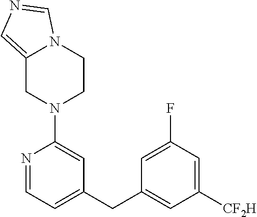 [SMILES: Fc1cc(Cc2ccnc(N3CCn4cncc4C3)c2)cc(C(F)F)c1]

To a sealed tube was added 2-bromo-4-(3-(difluoromethyl)-5-fluorobenzyl)pyridine (40 mg, 0.13 mmol), 5,6,7,8-tetrahydroimidazo[1,5-a]pyrazine (24 mg, 0.19 mmol), Pd₂(dba)₃ (12 mg, 0.013 mmol), Ruphos (12 mg, 0.025 mmol), tBuONa (24 mg, 0.25 mmol) and 1,4-dioxane (2 mL). The mixture was stirred at 100° C. for 1 h. Upon completion, the mixture was diluted by EtOAc and filtered through a pad of Celite®. The filtrate was concentrated in vacuo, and the residue was purified by automated column eluting with MeCN:H₂O (5% to 100%) using Biotage Selekt® unit to afford the title compound as a yellow oil (9.8 mg, 22%). ¹H NMR (400 MHz, Acetone-d6) δ 8.08 (d, J=5.1 Hz, 1H), 7.59 (br s, 1H), 7.36 (s, 1H), 7.29-7.18 (m, 2H), 7.03-6.73 (m, 3H), 6.60 (d, J=5.2 Hz, 1H), 4.76 (s, 2H), 4.19-4.16 (m, 2H), 4.10-4.05 (m, 2H), 4.04 (s, 2H). HRMS (ESI) m/z calcd for C₁₉H₁₈F₃N₄ [M+H]⁺: 359.1478; found: 359.1478.

### Example 25. Synthesis of 7-(4-(fluoro (3-fluoro-5-(trifluoromethyl)phenyl)methyl)pyridin-2-yl)-1-methyl-6,7-dihydroimidazo[1,5-a]pyrazin-8(5H)-one (Compound 81)

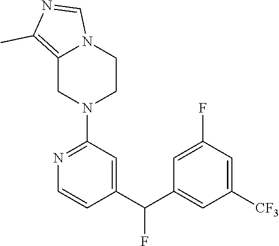 [SMILES: Cc1ncn2c1CN(c1cc(C(F)c3cc(F)cc(C(F)(F)F)c3)ccn1)CC2]

To a solution of 7-(4-((3-fluoro-5-(trifluoromethyl)phenyl) (hydroxy)methyl)pyridin-2-yl)-1-methyl-6,7-dihydroimidazo[1,5-a]pyrazin-8(5H)-one (20 mg, 0.05 mmol) in CH₂Cl₂ (2 mL) at 0° C. was added DAST (19 μL, 0.15 mmol) and the reaction mixture was stirred at RT for 16 h. The mixture was poured into a saturated aqueous solution of NaHCO₃ (20 mL) and extracted with CH₂Cl₂ (3× 20 mL). The combined organic layer was washed with brine, dried over MgSO₄, and concentrated in vacuo. The residue was purified by automated reverse phase column chromatography eluting with MeCN:H₂O (5% to 100%) using Biotage Selekt® unit to afford the title compound as a pale-yellow oil (5.1 mg, 25%). ¹H NMR (400 MHz, Acetone-d6) δ 8.46 (d, J=5.1 Hz, 1H), 8.19-8.18 (m, 1H), 7.75 (s, 2H), 7.65-7.57 (m, 2H), 7.26-7.25 (m, 1H), 6.90 (d, J=46.1 Hz, 1H), 4.51-4.41 (m, 4H), 2.45 (s, 3H). HRMS (ESI) m/z calcd for C₂₀H₁₆F₅N₄₀ [M+H]⁺: 423.1239; found: 423.1250.

### Example 26. Synthesis of 7-(4-(1-(3-fluoro-5-(trifluoromethyl)phenyl)ethyl)pyridin-2-yl)-1-methyl-6,7-dihydroimidazo[1,5-a]pyrazin-8(5H)-one (Compound 82)

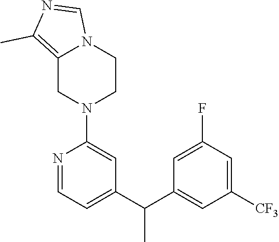 [SMILES: Cc1ncn2c1CN(c1cc(C(C)c3cc(F)cc(C(F)(F)F)c3)ccn1)CC2]

To a solution of 7-(4-(1-(3-fluoro-5-(trifluoromethyl)phenyl) vinyl)pyridin-2-yl)-1-methyl-6,7-dihydroimidazo[1,5-a]pyrazin-8(5H)-one (35 mg, 0.084 mmol) in MeOH (6 mL) was added 10% Pd/C (7 mg, 20% wt). The flask was filled with N₂ first and replaced with Hydrogen atmosphere using H₂ balloon. The reaction was vigorously stirred at rt for 16 h. After completion, the mixture was filtered through a pad of Celite®, washed with MeOH, and the solvent was concentrated in vacuo. The residue was purified by automated reverse phase column chromatography eluting with MeCN:H₂O (5% to 100%) using Biotage Selekt® unit to afford the title compound as a colorless solid (31 mg, 88%). HPLC 254 nm (purity)>98%. ¹H NMR (400 MHz, Acetone-d6) δ 8.33 (d, J=5.2 Hz, 1H), 8.04-8.03 (m, 1H), 7.63 (s, 1H), 7.58 (s, 1H), 7.49-7.45 (m, 1H), 7.40-7.37 (m, 1H), 7.12 (dd, J=5.2, 1.6 Hz, 1H), 4.49-4.39 (m, 5H), 2.45 (s, 3H), 1.74 (d, J=7.2 Hz, 3H). LC/MS (APCI): m/z=419.1 [M+H]⁺, RT 3.394 min. HRMS (ESI) m/z calcd for C₂₁H₁₉F₄N₄₀ [M+H]⁺: 419.1490; found: 419.1505.

### Example 27. Synthesis of 7-(4-(3-(difluoromethyl)-5-fluorobenzyl)pyridin-2-yl)-5,5-dimethyl-6,7-dihydroimidazo[1,5-a]pyrazin-8(5H)-one (Compound 83)

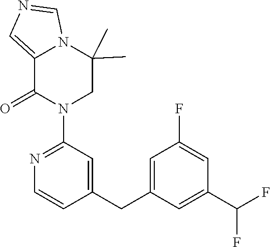 [SMILES: CC1(C)CN(c2cc(Cc3cc(F)cc(C(F)F)c3)ccn2)C(=O)c2cncn21]

To a stirred solution of 5,5-dimethyl-6,7-dihydroimidazo[1,5-a]pyrazin-8(5H)-one (0.140 g, 0.848 mmol, 1.0 equiv) and 2-chloro-4-(3-(difluoromethyl)-5-fluorobenzyl)pyridine (0.229 g, 0.847 mmol, 1.0 equiv) in dioxane (1.4 mL), was added Cs₂CO₃ (0.826 g, 2.54 mmol, 3.0 equiv) at room temperature then purged N₂ gas through the reaction mixture for 10 min. The catalyst Xantphos Pd-G3 (0.156 g, 0.169 mmol, 0.2 equiv) was added and the reaction mixture was heated under stirring at 80° C. for 2 h. After completion of the reaction as indicated by TLC (10% MeOH in DCM), the reaction mixture was diluted with water (20 mL) and extracted with EtOAc (3×15 mL). The combined organic layers were dried over Na₂SO₄ and concentrated under reduced pressure to afford the crude compound. The crude compound was purified by normal phase column chromatography (230-400 silica, 5% MeOH in DCM) to afford the title compound as a light brown solid (0.020 g, 6% yield); ¹H NMR (400 MHz, d₆-DMSO): δ ppm 8.36 (d, J=4.8 Hz, 1H), 8.11 (s, 1H), 7.89 (s, 1H), 7.67 (s, 1H), 7.40-7.35 (s, 2H), 7.31 (d, J=8.8 Hz, 1H), 7.14 (d, J=5.2 Hz, 1H), 7.03 (t, J=55.6 Hz, 1H), 4.22 (s, 2H), 4.11 (s, 2H), 1.57 (s, 6H); LCMS-02 (Method-A): RT 2.04 min, m/z 401.7 [M+H]⁺.

### Example 28. Synthesis of 7-(6-(3-(difluoromethyl)-5-fluorobenzyl)pyrimidin-4-yl)-5,6,7,8-tetrahydroimidazo[1,5-a]pyrazine (Compound 84)

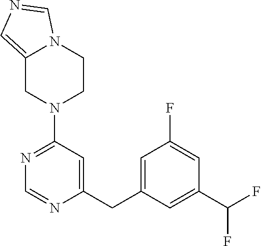 [SMILES: Fc1cc(Cc2cc(N3CCn4cncc4C3)ncn2)cc(C(F)F)c1]

To a stirred solution of 4-chloro-6-(3-(difluoro methyl)-5-fluorobenzyl)pyrimidine (0.1 g, 0.36 mmol, 1.0 equiv) and 5,6,7,8-tetrahydroimidazo[1,5-a]pyrazine (CAS: 297172-19-1) (0.03 g, 0.25 mmol, 0.7 equiv) in 1,4-dioxane (2.0 mL), was added NaOtBu (0.10 g, 1.10 mmol, 3.0 equiv) at room temperature then purge N₂ gas through the reaction mixture for 10 min. The catalyst Pd₂(dba₃) (0.03 g, 0.036 mmol, 0.1 equiv) and DavePhos (0.014 g, 0.036 mmol, 0.1 equiv) were added and the reaction mixture stirred at 80° C. for 3 h. After completion of the reaction as indicated by TLC (5% MeOH in DCM), the reaction mixture was filtered through a celite bed and washed with DCM (3× 10 mL). The filtrate was concentrated under reduced pressure to afford the crude material. The crude compound was purified by flash column chromatography using 230-400 silica as stationary phase (gradient elution 0-5% MeOH in DCM) to afford the crude material with enriched purity. The crude material was further purified by Prep-HPLC purification (Mobile Phase: (A) 0.05% HCOOH in H₂O (B) CH₃CN and Column (Waters X-Bridge Prep C8 (250 mm×19 mm 5 μm)) to afford the title compound as an off-white semi-solid (0.005 g, 4% yield). ¹H NMR (400 MHz, do-DMSO): δ ppm 8.48 (s, 1H), 7.62 (s, 1H), 7.36 (s, 2H), 7.27 (d, J=9.2 Hz, 1H), 7.02-6.99 (m, 2H), 6.80 (s, 1H), 4.81 (s, 2H), 4.15 (s, 2H), 4.01-3.97 (m, 4H); LCMS-02 (Method-A): RT 1.56 min, m/z 360.49 [M+H]⁺.

### Example 29. Synthesis of 7-(4-((3,4-dihydro-2H-benzo[b][1,4]oxazin-6-yl)oxy)pyridin-2-yl)-1-methyl-6,7-dihydroimidazo[1,5-a]pyrazin-8(5H)-one (Compound 93)

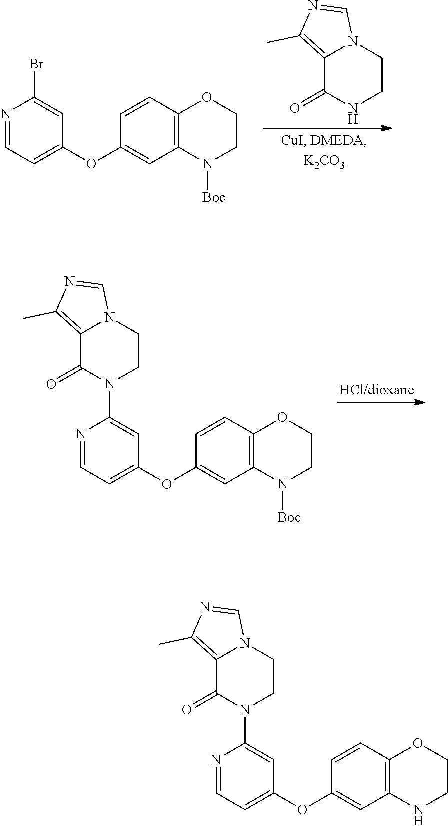 [SMILES: CC(C)(C)OC(=O)N1CCOc2ccc(Oc3ccnc(Br)c3)cc21 ; Cc1ncn2c1C(=O)NCC2 ; Cc1ncn2c1C(=O)N(c1cc(Oc3ccc4c(c3)N(C(=O)OC(C)(C)C)CCO4)ccn1)CC2 ; Cc1ncn2c1C(=O)N(c1cc(Oc3ccc4c(c3)NCCO4)ccn1)CC2]

Step A: To a solution of tert-butyl 6-((2-bromopyridin-4-yl)oxy)-2,3-dihydro-4H-benzo[b][1,4]oxazine-4-carboxylate (94.29 mg, 231.53 μmol, 1 eq.) and 1-methyl-6,7-dihydroimidazo[1,5-a]pyrazin-8(5H)-one (36.84 mg, 231.53 μmol, 1 eq.) in dioxane (7 mL) was added K₂CO₃ (128.00 mg, 926.14 μmol, 4 eq.), CuI (8.82 mg, 46.31 μmol, 0.2 eq.) and DMEDA (8.16 mg, 92.61 μmol, 9.97 μL, 0.4 eq.). The mixture was stirred at 110° C. for 16 h under N₂ atmosphere. The mixture was filtered and concentrated under reduced pressure to give a residue. The residue was purified by flash silica gel chromatography (0-4% methanol/DCM gradient) to give tert-butyl 6-((2-(1-methyl-8-oxo-5,6-dihydroimidazo[1,5-a]pyrazin-7 (8H)-yl)pyridin-4-yl)oxy)-2,3-dihydro-4H-benzo[b][1,4]oxazine-4-carboxylate (70 mg, 146.59 μmol, 63.31% yield) as a white solid: ¹H NMR (400 MHz, MeOH-d₄) δ=8.35-8.21 (m, 1H), 7.84-7.63 (m, 2H), 7.38 (d, J=1.2 Hz, 1H), 6.94 (d, J=9.2 Hz, 1H), 6.83-6.75 (m, 2H), 4.37 (s, 4H), 4.24 (t, J=4.4 Hz, 2H), 3.87 (t, J=4.4 Hz, 2H), 2.47 (s, 3H), 1.49 (s, 9H)

Step B: To a solution of tert-butyl 6-((2-(1-methyl-8-oxo-5,6-dihydroimidazo[1,5-a]pyrazin-7 (8H)-yl)pyridin-4-yl)oxy)-2,3-dihydro-4H-benzo[b][1,4]oxazine-4-carboxylate (70 mg, 146.59 μmol, 1 eq.) in DCM (1 mL) was added HCl/dioxane (2 M, 3.50 mL, 47.75 eq.). The mixture was stirred at 40° C. for 5 h. The residue was treated with ice-water (20 mL) and EtOAc (20 mL). The leaving aqueous layer was adjusted with sat. NaHCO₃ until pH to 8-9. The mixture was filtered and the filter cake was washed with water (5 mL×2). The filter cake was dried under vacuum (30° C.) to give the crude product. The residue was purified by prep-HPLC (column: Welch Xtimate C18 150*25 mm*5 μm; mobile phase: [H₂O (10 mM NH₄HCO₃)-ACN]; gradient: 21%-51% ACN over 11.0 min) to give 7-(4-((3,4-dihydro-2H-benzo[b][1,4]oxazin-6-yl)oxy)pyridin-2-yl)-1-methyl-6,7-dihydroimidazo[1,5-a]pyrazin-8(5H)-one (21 mg, 55.24 μmol, 37.68% yield) as a white solid. LCMS [M+H]⁺=378.2; ¹H NMR (400 MHz, DMSO-d₆) δ=8.27 (d, J=5.6 Hz, 1H), 7.77 (s, 1H), 7.39 (d, J=2.4 Hz, 1H), 6.76-6.67 (m, 2H), 6.34 (d, J=2.8 Hz, 1H), 6.23 (dd, J=2.8, 8.8 Hz, 1H), 6.06 (br s, 1H), 4.30 (s, 4H), 4.12 (br t, J=4.0 Hz, 2H), 3.31-3.28 (m, 2H), 2.37 (s, 3H).

### Example 30. Synthesis of 7-(4-((4-fluoro-3-methyl-1H-indazol-6-yl)oxy)pyridin-2-yl)-1-methyl-6,7-dihydroimidazo[1,5-a]pyrazin-8(5H)-one (Compound 95)

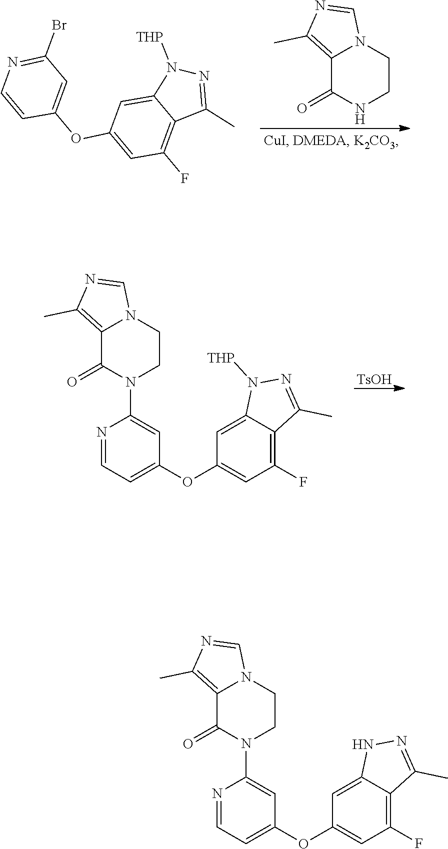

Step A: To a solution of 6-((2-bromopyridin-4-yl)oxy)-4-fluoro-3-methyl-1-(tetrahydro-2H-pyran-2-yl)-1H-indazole (200 mg, 492.31 μmol, 1 eq.) and 1-methyl-6,7-dihydroimidazo[1,5-a]pyrazin-8(5H)-one (81.86 mg, 541.54 μmol, 1.1 eq.) in dioxane (20 mL) was added K₂CO₃ (272.17 mg, 1.97 mmol, 4 eq.), DMEDA (17.36 mg, 196.92 μmol, 21.20 μL, 0.4 eq.) and CuI (18.75 mg, 98.46 μmol, 0.2 eq.), the residue was purged with N₂ three times and the mixture was stirred at 110° C. for 16 h under N₂ atmosphere. Then the mixture was cooled and concentrated under reduced pressure to give a residue. The residue was purified by flash silica gel chromatography (0-4% MeOH/DCM gradient) to yield 7-(4-((4-fluoro-3-methyl-1-(tetrahydro-2H-pyran-2-yl)-1H-indazol-6-yl)oxy)pyridin-2-yl)-1-methyl-6,7-dihydroimidazo[1,5-a]pyrazin-8(5H)-one (240 mg, 488.56 μmol, 99.24% yield) as a yellow solid. LCMS [M+H]⁺=477.2; ¹H NMR (400 MHz, DMSO-d₆) δ=8.37 (d, J=5.6 Hz, 1H), 7.54 (d, J=2.4 Hz, 1H), 7.44 (d, J=1.6 Hz, 1H), 6.93-6.86 (m, 2H), 5.79-5.73 (m, 1H), 4.37 (br s, 2H), 4.33-4.25 (m, 2H), 3.94-3.83 (m, 1H), 3.77-3.65 (m, 1H), 3.17 (d, J=5.6 Hz, 1H), 2.58 (s, 3H), 2.41-2.28 (m, 4H), 2.03-1.91 (m, 2H), 1.75-1.65 (m, 1H), 1.58-1.51 (m, 2H); ¹⁹F NMR (376 MHz, DMSO-d₆) δ=−117.751.

Step B: To a solution of 7-(4-((4-fluoro-3-methyl-1-(tetrahydro-2H-pyran-2-yl)-1H-indazol-6-yl)oxy)pyridin-2-yl)-1-methyl-6,7-dihydroimidazo[1,5-a]pyrazin-8(5H)-one (80 mg, 167.89 μmol, 1 eq.) in THF (0.8 mL) and H₂O (0.8 mL) was added TsOH (57.82 mg, 335.78 μmol, 2 eq.). The mixture was stirred at 80° C. for 5 h. The mixture was concentrated under reduced pressure to give a residue. The residue was purified by prep-HPLC (column: 55-Boston Prime C18 150×30 mm, 5 μm; mobile phase: [H₂O (0.05% NH₃H₂O+10 mM NH₄HCO₃)-ACN]; gradient: 36%-66% ACN over 10.0 min), followed by lyophilization to yield 7-(4-((4-fluoro-3-methyl-1H-indazol-6-yl)oxy)pyridin-2-yl)-1-methyl-6,7-dihydroimidazo[1,5-a]pyrazin-8(5H)-one (35 mg, 89.20 μmol, 53.13% yield) as a white solid. LCMS [M+H]⁺=393.1; ¹H NMR (400 MHz, DMSO-d₆) δ=13.00 (br s, 1H), 8.35 (d, J=5.6 Hz, 1H), 7.78 (s, 1H), 7.51 (d, J=2.0 Hz, 1H), 7.07 (d, J=1.6 Hz, 1H), 6.94-6.84 (m, 1H), 6.78 (br d, J=11.2 Hz, 1H), 4.36-4.21 (m, 4H), 2.57 (s, 3H), 2.35 (s, 3H); ¹⁹F NMR (376 MHz, DMSO-d₆) δ=−117.942.

### Example 31. Synthesis of 5-(4-(3-(difluoromethyl)-5-fluorobenzyl)pyridin-2-yl)-3-(hydroxymethyl)-1-methyl-1,5,6,7-tetrahydro-4H-pyrazolo[4,3-c]pyridin-4-one (Compound 98)

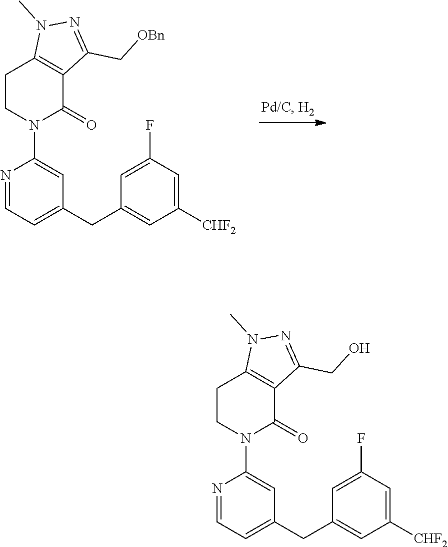 [SMILES: Cn1nc(COCc2ccccc2)c2c1CCN(c1cc(Cc3cc(F)cc(C(F)F)c3)ccn1)C2=O ; Cn1nc(CO)c2c1CCN(c1cc(Cc3cc(F)cc(C(F)F)c3)ccn1)C2=O]

Step A: To a solution of 3-((benzyloxy)methyl)-5-(4-(3-(difluoromethyl)-5-fluorobenzyl)pyridin-2-yl)-1-methyl-1,5,6,7-tetrahydro-4H-pyrazolo[4,3-c]pyridin-4-one (100 mg, 187.56 μmol, 1 eq.) in MeOH (50 mL) was added Pd/C (1.00 g, 939.67 μmol, 10% purity, 5.01 eq.) under N₂ atmosphere. The suspension was degassed and purged with H₂ three times. The mixture was stirred at 50° C. for 16 h under H₂ (30 psi) atmosphere. Then the reaction mixture was filtered, and the filtrate was concentrated under reduced pressure to give a residue. The residue was purified by prep-HPLC (column: Welch Xtimate C18 150*25 mm*5 μm; mobile phase: [H₂O (10 mM NH₄HCO₃)—ACN]; gradient: 30%-62% ACN over 11.0 min) to yield 5-(4-(3-(difluoromethyl)-5-fluorobenzyl)pyridin-2-yl)-3-(hydroxymethyl)-1-methyl-1,5,6,7-tetrahydro-4H-pyrazolo[4,3-c]pyridin-4-one (20.05 mg, 48.15 μmol, 25.67% yield) as a white solid. LCMS [M+H]⁺=417.1; ¹H NMR (400 MHz, DMSO-d₆) δ=8.34 (d, J=5.2 Hz, 1H), 7.65 (s, 1H), 7.36 (s, 2H), 7.29 (d, J=9.2 Hz, 1H), 7.16-6.87 (m, 2H), 5.10 (t, J=6.4 Hz, 1H), 4.57 (d, J=6.0 Hz, 2H), 4.23 (t, J=6.4 Hz, 2H), 4.08 (s, 2H), 3.75 (s, 3H), 3.06 (t, J=6.8 Hz, 2H); ¹⁹F NMR (376 MHz, DMSO-d₆) δ=−110.260, −111.829.

### Example 32. Synthesis of 1-methyl-7-(4-((2-(methylamino)pyridin-4-yl)oxy)pyridin-2-yl)-6,7-dihydroimidazo[1,5-a]pyrazin-8(5H)-one (Compound 99)

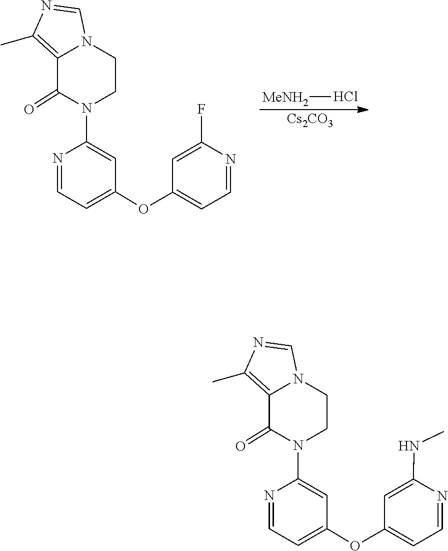

Step A: 7-(4-((2-fluoropyridin-4-yl)oxy)pyridin-2-yl)-1-methyl-6,7-dihydroimidazo[1,5-a]pyrazin-8(5H)-one (100 mg, 294.70 μmol, 1 eq.), methylamine hydrochloride (99.49 mg, 1.47 mmol, 5 eq.) and Cs₂CO₃ (384.08 mg, 1.18 mmol, 4 eq.) were taken up into a microwave tube in NMP (1.5 mL). The sealed tube was heated at 150° C. for 3 h under microwave. The mixture was quenched by the addition of water (20 mL) and extracted with EtOAc (20 mL×3). The combined organic phase was washed with brine (50 mL), dried over anhydrous Na₂SO₄, filtered and concentrated in vacuum to yield a residue. The residue was purified by prep-HPLC (column: Welch Xtimate C18 150*25 mm*5 μm; mobile phase: [H₂O (10 mM NH₄HCO₃)-ACN]; gradient: 18%-48% ACN over 11.0 min), followed by lyophilization to yield 1-methyl-7-(4-((2-(methylamino)pyridin-4-yl)oxy)pyridin-2-yl)-6,7-dihydroimidazo[1,5-a]pyrazin-8 (5/)-one (48.11 mg, 135.86 μmol, 46.10% yield) as a white solid. LCMS [M+H]⁺=351.3; ¹H NMR (400 MHz, DMSO-d₆) δ=8.39 (d, J=5.6 Hz, 1H), 8.00 (d, J=5.6 Hz, 1H), 7.79 (s, 1H), 7.60 (d, J=2.0 Hz, 1H), 6.94 (dd, J=2.0, 5.6 Hz, 1H), 6.67 (q, J=4.4 Hz, 1H), 6.28 (dd, J=2.0, 5.6 Hz, 1H), 6.11 (d, J=2.0 Hz, 1H), 4.40-4.25 (m, 4H), 2.76 (d, J=4.8 Hz, 3H), 2.38 (s, 3H).

### Example 33. Synthesis of 7-(4-((2-fluoropyridin-4-yl)oxy)pyridin-2-yl)-1-methyl-6,7-dihydroimidazo[1,5-a]pyrazin-8(5H)-one (Compound 101)

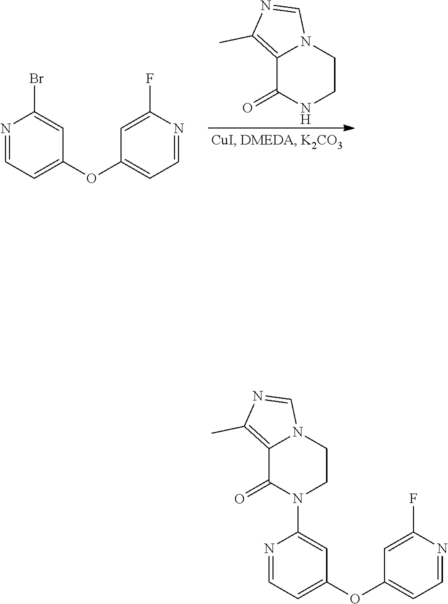 [SMILES: Fc1cc(Oc2ccnc(Br)c2)ccn1 ; Cc1ncn2c1C(=O)NCC2 ; Cc1ncn2c1C(=O)N(c1cc(Oc3ccnc(F)c3)ccn1)CC2]

Step A: A mixture of 1-methyl-6,7-dihydroimidazo[1,5-a]pyrazin-8(5H)-one (539.34 mg, 3.57 mmol, 1.2 eq.), 2-bromo-4-((2-fluoropyridin-4-yl)oxy)pyridine (800 mg, 2.97 mmol, 1 eq.), K₂CO₃ (1.23 g, 8.92 mmol, 3 eq.), DMEDA (104.84 mg, 1.19 mmol, 128.01 μL, 0.4 eq.) and CuI (113.25 mg, 594.64 μmol, 0.2 eq.) in dioxane (24 mL) was degassed and purged with N₂ three times, and then the mixture was stirred at 110° C. for 14 h under N₂ atmosphere. The reaction mixture was filtered and concentrated under reduced pressure to give a residue. The residue was purified by flash silica gel chromatography (DCM to DCM/MeOH=20/1, TLC: DCM/MeOH=10/1, R_{f}=0.3) to yield 7-(4-((2-fluoropyridin-4-yl)oxy)pyridin-2-yl)-1-methyl-6,7-dihydroimidazo[1,5-a]pyrazin-8(5H)-one (800 mg, 2.36 mmol, 79.30% yield) as a yellow solid. LCMS [M+H]⁺=340.1; ¹H NMR (400 MHz, DMSO-d₆) δ=8.49 (d, J=5.6 Hz, 1H), 8.27 (d, J=5.6 Hz, 1H), 7.81 (s, 1H), 7.75 (d, J=2.0 Hz, 1H), 7.16-7.12 (m, 1H), 7.11-7.07 (m, 1H), 7.01 (d, J=1.2 Hz, 1H), 4.45-4.28 (m, 4H), 2.39 (s, 3H).

### Example 34. Synthesis of 7-(2-(3-(difluoromethyl)-5-fluorobenzyl) pyrazolo[1,5-a]pyrazin-4-yl)-1-methyl-6,7-dihydroimidazo[1,5-a]pyrazin-8(5H)-one (Compound 107)

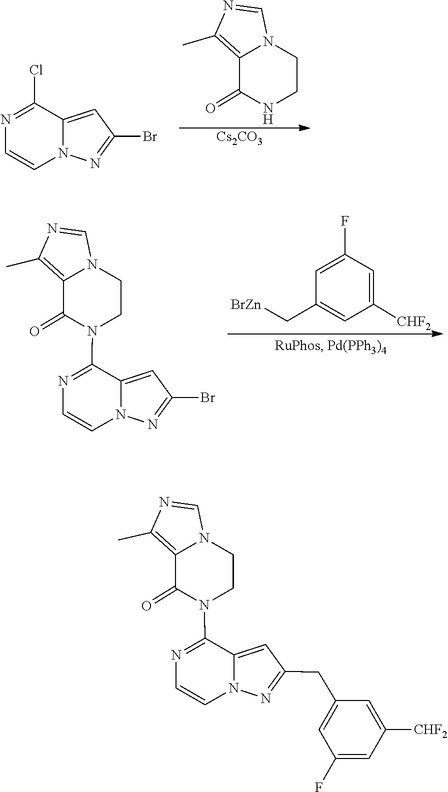

Step A: To a solution of 1-methyl-6,7-dihydroimidazo[1,5-a]pyrazin-8(5H)-one (214.59 mg, 1.42 mmol, 1.1 eq.) in DMF (5 mL) was added Cs₂CO₃ (840.95 mg, 2.58 mmol, 2 eq.) and 2-bromo-4-chloropyrazolo[1,5-a]pyrazine (300 mg, 1.29 mmol, 1 eq.). The mixture was stirred at 80° C. for 1 h under N₂. The reaction mixture was poured into water (20 mL) and extracted with EtOAc (30 mL×3). The combined organics were washed with water (30 mL×3) and brine (30 mL×3), then dried over Na₂SO₄, filtered and concentrated under reduced pressure. The residue was purified by flash silica gel chromatography (DCM to DCM/MeOH=93/7) to give 7-(2-bromopyrazolo[1,5-a]pyrazin-4-yl)-1-methyl-6,7-dihydroimidazo[1,5-a]pyrazin-8(5H)-one (350 mg, 1.01 mmol, 78.12% yield) as a white solid: ¹H NMR (400 MHz, DMSO-d₆) δ=8.70 (d, J=4.8 Hz, 1H), 7.86 (s, 1H), 7.83 (d, J=4.8 Hz, 1H), 6.84 (s, 1H), 4.42-4.33 (m, 2H), 4.29-4.22 (m, 2H), 2.41 (s, 3H).

Step B: Dissolve 1-(bromomethyl)-3-(difluoromethyl)-5-fluorobenzene (1 g, 4.18 mmol, 1 eq.) in anhydrous THF (8.4 mL) to form a 0.5 M THF solution. Pass this solution at flow rate of 0.5 mL/min through a preinstalled zinc powder column that has been flushed with anhydrous THF and activated with a solution of TMS-C1 (0.8 mL) and 1,2-dibromoethane (0.2 mL) in THF (10 mL) at 50° C. at flow rate 1 mL/min. The resulting yellow solution of (3-(difluoromethyl)-5-fluorobenzyl) zinc (II) bromide was collected and titrated with a solution of iodine before use.

To a solution of 7-(2-bromopyrazolo[1,5-a]pyrazin-4-yl)-1-methyl-6,7-dihydroimidazo[1,5-a]pyrazin-8(5H)-one (180 mg, 518.48 μmol, 1 eq.) in THF (10 mL) was added RuPhos (2-dicyclohexylphosphino-2′,6′-diisopropoxybiphenyl, 24.19 mg, 51.85 μmol, 0.1 eq.), Pd(PPh₃)₄ (59.91 mg, 51.85 μmol, 0.1 eq.) and 3-(difluoromethyl)-5-fluorobenzyl) zinc (II) bromide (0.24 M, 3.24 mL, 1.5 eq.) at 0° C. The reaction mixture was stirred at 60° C. for 12 h under N₂. The reaction mixture was filtered and concentrated under reduced pressure. The residue was purified by prep-HPLC (column: 55-Boston Prime C18 150×30 mm, 5 μm; mobile phase: [H₂O (0.05% NH₃H₂O+10 mM NH₄HCO₃)-ACN]; gradient: 34%-64% ACN over 10.0 min) to give 7-(2-(3-(difluoromethyl)-5-fluorobenzyl) pyrazolo[1,5-a]pyrazin-4-yl)-1-methyl-6,7-dihydroimidazo[1,5-a]pyrazin-8(5H)-one (48 mg, 111.66 μmol, 21.54% yield) as a white solid. LCMS [M+H]⁺=427.2; ¹H NMR (400 MHz, DMSO-d₆) δ=8.66 (d, J=4.8 Hz, 1H), 7.84 (s, 1H), 7.74 (d, J=4.8 Hz, 1H), 7.41-7.34 (m, 2H), 7.27 (d, J=8.8 Hz, 1H), 7.16-6.87 (m, 1H), 6.56 (s, 1H), 4.38-4.30 (m, 2H), 4.26 (s, 2H), 4.24-4.19 (m, 2H), 2.40 (s, 3H); ¹⁹F NMR (376 MHz, DMSO-d₆) δ=−110.222, −112.078.

### Example 35. Synthesis of 5-(4-(3-(difluoromethyl)-5-fluorobenzyl)pyridin-2-yl)-1,3-dimethyl-1,5-dihydro-4H-pyrazolo[4,3-c]pyridin-4-one (Compound 109) and 5-(4-(3-(difluoromethyl)-5-fluorobenzoyl)pyridin-2-yl)-1,3-dimethyl-1,5-dihydro-4H-pyrazolo[4,3-c]pyridin-4-one (Compound 111)

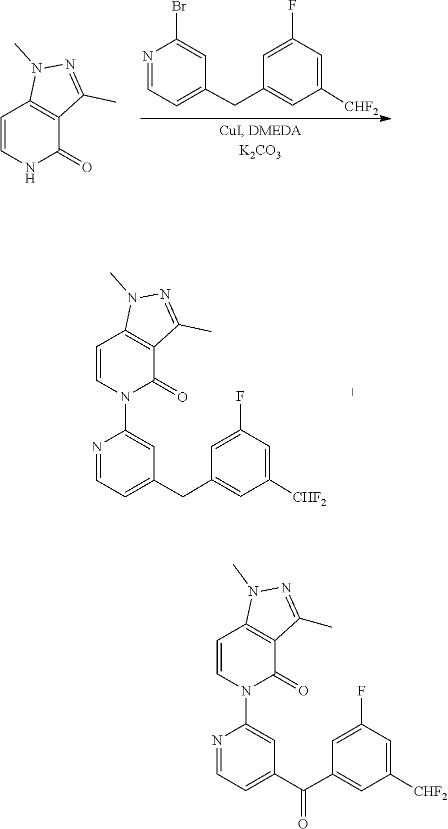

Step A: To a solution of 1,3-dimethyl-1,5-dihydro-4H-pyrazolo[4,3-c]pyridin-4-one (56.78 mg, 347.97 μmol, 1.1 eq.) in dioxane (3 mL) was added CuI (12.05 mg, 63.27 μmol, 0.2 eq.), K₂CO₃ (131.16 mg, 949.02 μmol, 3 eq.), DMEDA (N,N′-dimethylethylenediamine, 11.15 mg, 126.54 μmol, 13.62 μL, 0.4 eq.) and 2-bromo-4-(3-(difluoromethyl)-5-fluorobenzyl)pyridine (100 mg, 316.34 μmol, 1 eq.). The mixture was stirred at 110° C. for 16 h under N₂. The reaction mixture was concentrated and then water (10 mL) was added. The mixture was extracted with EtOAc (10 mL×3). The combined organic layers were washed with brine (30 mL) and dried over Na₂SO₄ and filtered. The filtrate was concentrated under reduced pressure to give a residue, which was purified by flash silica gel chromatography (hexanes to hexanes/EtOAc=1/9, TLC: hexane/EtOA c=0/1, R_{f}=0.36) to give a residue. The residue was added MeOH (2 mL) and filtered. The filter cake was collected to give 5-(4-(3-(difluoromethyl)-5-fluorobenzoyl)pyridin-2-yl)-1,3-dimethyl-1,5-dihydro-4H-pyrazolo[4,3-c]pyridin-4-one (20.1 mg, 48.54 μmol, 15.35% yield) as a white solid. LCMS [M+H]⁺=413.2; ¹H NMR (400 MHz, DMSO-d₆) δ=8.83 (d, J=5.2 Hz, 1H), 8.08 (s, 1H), 7.92-7.81 (m, 4H), 7.73 (dd, J=1.2, 4.8 Hz, 1H), 7.17 (t, J=55.2 Hz, 1H), 6.82 (d, J=8.0 Hz, 1H), 3.90 (s, 3H), 2.47 (s, 3H); ¹⁹F NMR (400 MHz, DMSO-d₆) δ=−109.918, −110.831.

The filtrate was purified by prep-HPLC (column: Welch Xtimate C18 150*25 mm*5 μm; mobile phase: [H₂O (0.225% FA)-ACN]; gradient: 45%-65% ACN over 11.0 min, flow rate: 25.00 mL/min) to give 5-(4-(3-(difluoromethyl)-5-fluorobenzyl)pyridin-2-yl)-1,3-dimethyl-1,5-dihydro-4H-pyrazolo[4,3-c]pyridin-4-one (20.05 mg, 50.33 μmol, 15.91% yield) as a white solid. LCMS [M+H]⁺=399.1; ¹H NMR (400 MHz, DMSO-d₆) δ=8.49 (d, J=5.2 Hz, 1H), 7.75-7.66 (m, 2H), 7.43-7.39 (m, 2H), 7.37 (dd, J=1.2, 4.8 Hz, 1H), 7.30 (br d, J=7.2 Hz, 1H), 7.02 (t, J=54.8 Hz, 1H), 6.74 (d, J=7.6 Hz, 1H), 4.16 (s, 2H), 3.88 (s, 3H), 2.46 (s, 3H); ¹⁹F NMR (400 MHz, DMSO-d₆) δ=−110.294, −111.708.

### Example 36. Synthesis of 7-(4-((5-(ethylamino)pyridin-3-yl)oxy)pyridin-2-yl)-1-methyl-6,7-dihydroimidazo[1,5-a]pyrazin-8 (5H)-one (Compound 120)

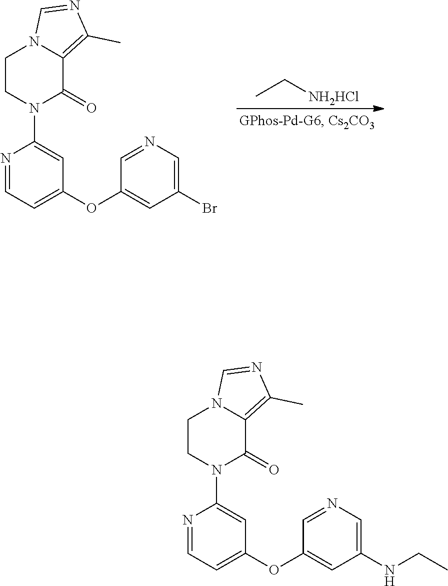

Step A: To a solution of 7-(4-((5-bromopyridin-3-yl)oxy)pyridin-2-yl)-1-methyl-6,7-dihydroimidazo[1,5-a]pyrazin-8(5H)-one (100 mg, 249.86 μmol, 1 eq.) and EtNH₂—HCl (81.50 mg, 999.43 μmol, 4 eq.) in dioxane (2 mL) was added Cs₂CO₃ (569.86 mg, 1.75 mmol, 7 eq.) and GPhos Pd G6 (CAS #2489525-81-5, 23.60 mg, 24.99 μmol, 0.1 eq.). The mixture was stirred at 125° C. for 1.5 h in microwave reactor. The reaction mixture was diluted with brine (10 mL) and extracted with DCM (50 mL×3). The combined organic layers were dried over sodium sulfate, filtered and concentrated under reduced pressure to give a residue. The residue was purified by prep-HPLC (column: 55-Boston Prime C18 150×30 mm, 5 μm; mobile phase: [H₂O (0.05% NH₃H₂O+10 mM NH₄HCO₃)-ACN]; gradient: 30%-61% ACN over 10.0 min), followed by lyophilization to yield 7-(4-((5-(ethylamino)pyridin-3-yl)oxy)pyridin-2-yl)-1-methyl-6,7-dihydroimidazo[1,5-a]pyrazin-8(5H)-one (23.93 mg, 65.01 μmol, 26.02% yield) as a white solid. LCMS M+H]+=365.2; ¹H NMR (400 MHz, DMSO-d₆) δ=8.33 (d, J=5.6 Hz, 1H), 7.89 (d, J=2.4 Hz, 1H), 7.78 (s, 1H), 7.62 (d, J=2.4 Hz, 1H), 7.48 (d, J=2.0 Hz, 1H), 6.84 (dd, J=2.4, 5.6 Hz, 1H), 6.69 (t, J=2.0 Hz, 1H), 6.23-6.14 (m, 1H), 4.37-4.26 (m, 4H), 3.11-3.01 (m, 2H), 2.37 (s, 3H), 1.16 (t, J=7.2 Hz, 3H).

### Example 37. Synthesis of 7-(4-(3-fluoro-5-(trifluoromethyl) phenoxy)pyridin-2-yl)-3-(hydroxymethyl)-1-methyl-6,7-dihydroimidazo[1,5-a]pyrazin-8(5H)-one (Compound 128)

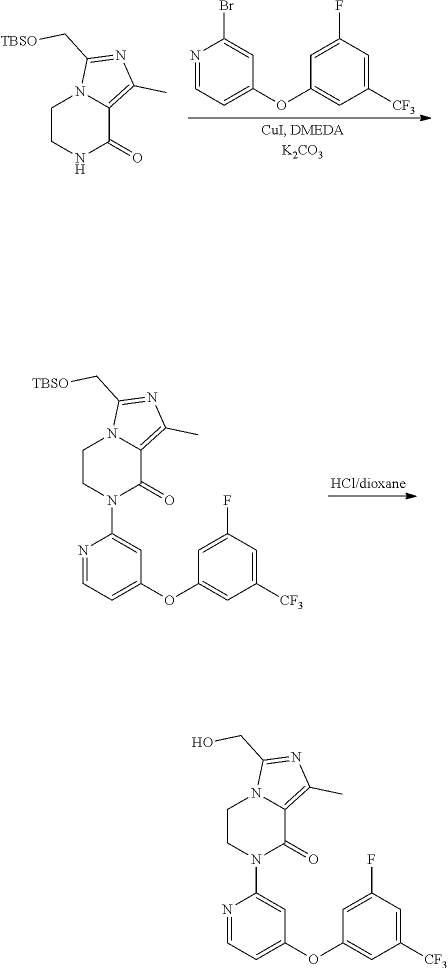

Step A: A mixture of 3-(((tert-butyldimethylsilyl)oxy)methyl)-1-methyl-6,7-dihydroimidazo[1,5-a]pyrazin-8(5H)-one (100 mg, 338.46 μmol, 1 eq.), 2-bromo-4-(3-fluoro-5-(trifluoromethyl) phenoxy)pyridine (136.50 mg, 406.16 μmol, 1.2 eq.), CuI (25.78 mg, 135.39 μmol, 0.4 eq.), K₂CO₃ (140.33 mg, 1.02 mmol, 3 eq.) and DMEDA (23.87 mg, 270.77 μmol, 29.14 μL, 0.8 eq.) in dioxane (8 mL) was degassed and purged with N₂ three times. Then the mixture was heated and stirred at 100° C. for 5 h under N₂ atmosphere. The reaction mixture was filtered and the filtrate was concentrated under reduced pressure to give 3-(((tert-butyldimethylsilyl)oxy)methyl)-7-(4-(3-fluoro-5-(trifluoromethyl) phenoxy)pyridin-2-yl)-1-methyl-6,7-dihydroimidazo[1,5-a]pyrazin-8 (5/I)-one (150 mg, 217.94 μmol, 64.39% yield) as a blue solid. LCMS [M+H]⁺=551.5.

Step B: A solution of 3-(((tert-butyldimethylsilyl)oxy)methyl)-7-(4-(3-fluoro-5-(trifluoromethyl) phenoxy)pyridin-2-yl)-1-methyl-6,7-dihydroimidazo[1,5-a]pyrazin-8(5H)-one (150 mg, 272.42 μmol, 1 eq.) in HCl/dioxane (5 mL) was stirred at 20° C. for 0.5 h. The reaction mixture was poured into aq. sat. NaHCO₃ (20 mL) and extracted with EtOAc (30 mL×3). The combined organic layers were washed with brine (30 mL×3) and dried over Na₂SO₄, filtered and concentrated under reduced pressure. The residue was purified by prep-TLC (SiO₂, DCM/MeOH=10/1) to give 7-(4-(3-fluoro-5-(trifluoromethyl) phenoxy)pyridin-2-yl)-3-(hydroxymethyl)-1-methyl-6,7-dihydroimidazo[1,5-a]pyrazin-8(5H)-one (53.57 mg, 119.13 μmol, 43.73% yield) as a white solid. LCMS [M+H]⁺=437.2; ¹H NMR (400 MHz, DMSO-d₆) δ=8.40 (d, J=5.6 Hz, 1H), 7.65 (d, J=8.4 Hz, 1H), 7.62 (d, J=2.0 Hz, 1H), 7.55 (d, J=9.6 Hz, 1H), 7.49 (s, 1H), 6.92 (dd, J=2.4, 5.6 Hz, 1H), 5.47 (t, J=5.6 Hz, 1H), 4.51 (d, J=5.6 Hz, 2H), 4.39-4.25 (m, 4H), 2.36 (s, 3H); ¹⁹F NMR (376 MHz, DMSO-d6) δ=−61.286, −106.959.

### Example 38. Synthesis of 5-(4-(3-fluoro-5-(trifluoromethyl) phenoxy)pyridin-2-yl)-1-(2-hydroxyethyl)-3-methyl-1,5,6,7-tetrahydro-4H-pyrazolo[4,3-c]pyridin-4-one (Compound 129)

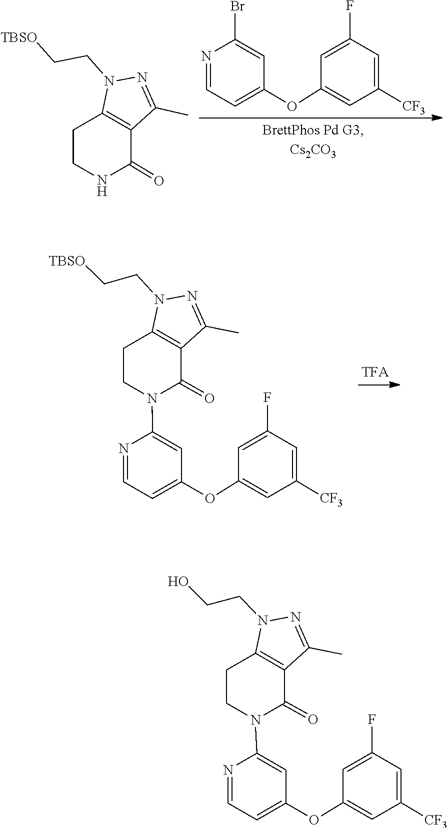

Step A: To a solution of 1-(2-((tert-butyldimethylsilyl)oxy)ethyl)-3-methyl-1,5,6,7-tetrahydro-4H-pyrazolo[4,3-c]pyridin-4-one (140 mg, 452.37 μmol, 1.01 eq.) and 2-bromo-4-(3-fluoro-5-(trifluoromethyl) phenoxy)pyridine (150 mg, 446.32 μmol, 1 eq.) in dioxane (15 mL) was added Cs₂CO₃ (436.26 mg, 1.34 mmol, 3 eq.) and BrettPhos Pd G3 (80.92 mg, 89.26 μmol, 0.2 eq.) under N₂ atmosphere. The mixture was stirred at 90° C. for 2 h under N₂ atmosphere. The mixture was then cooled and concentrated under vacuum to yield a residue. The residue was purified by flash silica gel chromatography (hexane to hexane/EtOAc=75/25, TLC: Hexane/EtOAc=1/1, R_{f}=0.50) to yield 1-(2-((tert-butyldimethylsilyl)oxy)ethyl)-5-(4-(3-fluoro-5-(trifluoromethyl) phenoxy)pyridin-2-yl)-3-methyl-1,5,6,7-tetrahydro-4H-pyrazolo[4,3-c]pyridin-4-one (220 mg, 350.66 μmol, 78.57% yield) as a yellow solid. LCMS [M+H]⁺=565.1; ¹H NMR (400 MHz, DMSO-d₆) δ=8.38 (d, J=5.6 Hz, 1H), 7.63 (d, J=8.4 Hz, 1H), 7.56-7.50 (m, 1H), 7.46 (s, 1H), 7.42 (d, J=2.4 Hz, 1H), 6.88 (dd, J=2.4, 5.6 Hz, 1H), 4.26 (t, J=6.4 Hz, 2H), 4.10 (t, J=4.8 Hz, 2H), 3.94-3.84 (m, 2H), 3.03 (t, J=6.4 Hz, 2H), 2.31 (s, 3H), 0.81-0.76 (m, 9H), −0.06-0.10 (m, 6H).

Step B: To a solution of 1-(2-((tert-butyldimethylsilyl)oxy)ethyl)-5-(4-(3-fluoro-5-(trifluoromethyl) phenoxy)pyridin-2-yl)-3-methyl-1,5,6,7-tetrahydro-4H-pyrazolo[4,3-c]pyridin-4-one (200 mg, 318.78 μmol, 1 eq.) in DCM (3 mL) was added TFA (9.21 g, 80.77 mmol, 6 mL, 253.38 eq.) at 0° C. The mixture was stirred at 20° C. for 4 h. The mixture was concentrated under vacuum, adjusted to pH 7-8 with sat. aq. NaHCO₃ and extracted with EtOAc (40 mL×3). The combined organics were washed with brine (60 mL), dried over anhydrous Na₂SO₄, filtered and concentrated under vacuum to yield a residue. The residue was purified by prep-HPLC (column: Welch Xtimate C18 150*25 mm*5 μm; mobile phase: [H₂O (10 mM NH₄HCO₃)-ACN]; gradient: 36%-66% ACN over 11.0 min) and prep-HPLC (column: Phenomenex Synergi C18 150*30 mm*4 μm; mobile phase: [H₂O (0.225% FA)-ACN]; gradient: 40%-70% ACN over 10.0 min), followed by lyophilization to yield 5-(4-(3-fluoro-5-(trifluoromethyl) phenoxy)pyridin-2-yl)-1-(2-hydroxyethyl)-3-methyl-1,5,6,7-tetrahydro-4H-pyrazolo[4,3-c]pyridin-4-one (69.4 mg, 154.09 μmol, 48.34% yield) as a white solid. LCMS [M+H]⁺=451.2; ¹H NMR (400 MHz, DMSO-d₆) δ=8.38 (d, J=5.6 Hz, 1H), 7.63 (d, J=8.4 Hz, 1H), 7.52 (d, J=9.6 Hz, 1H), 7.46 (d, J=2.4 Hz, 2H), 6.87 (dd, J=2.4, 5.6 Hz, 1H), 4.91 (t, J=5.6 Hz, 1H), 4.27 (t, J=6.4 Hz, 2H), 4.05 (t, J=5.2 Hz, 2H), 3.69 (q, J=5.2 Hz, 2H), 3.05 (t, J=6.4 Hz, 2H), 2.31 (s, 3H); ¹⁹F NMR (376 MHz, DMSO-d₆) δ=−61.299, −107.010.

### Example 39. Synthesis of 5-(4-(3-fluoro-5-(trifluoromethyl) phenoxy)pyridin-2-yl)-1-(2-hydroxyethyl)-3-methyl-1,5-dihydro-4H-pyrazolo[4,3-c]pyridin-4-one (Compound 130)

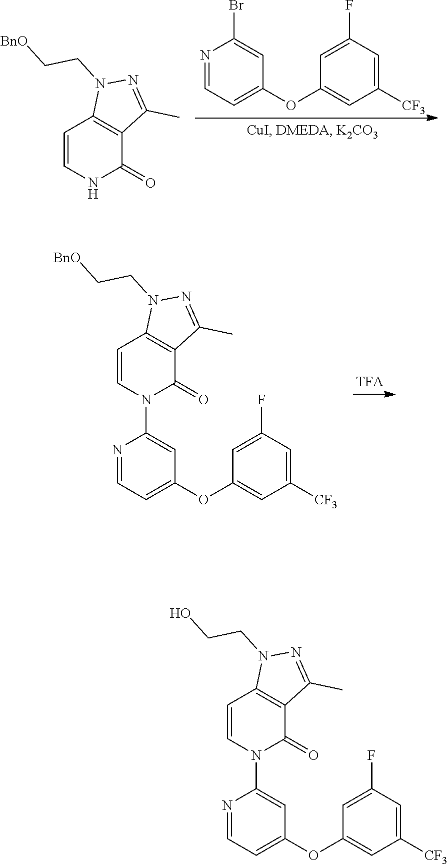

Step A: A mixture of 1-(2-(benzyloxy)ethyl)-3-methyl-1,5-dihydro-4H-pyrazolo[4,3-c]pyridin-4-one (100 mg, 352.95 μmol, 1 eq.), 2-bromo-4-(3-fluoro-5-(trifluoromethyl) phenoxy)pyridine (118.62 mg, 352.95 μmol, 1 eq.), CuI (13.44 mg, 70.59 μmol, 0.2 eq.), DMEDA (12.45 mg, 141.18 μmol, 15.20 μL, 0.4 eq.) and K₂CO₃ (146.34 mg, 1.06 mmol, 3 eq.) in dioxane (3 mL) was degassed and purged with N₂ three times, then the mixture was stirred at 130° C. for 2 h under N₂ atmosphere. The reaction mixture was cooled and diluted with EtOAc (30 mL), washed by H₂O (10 mL) and brine (10 mL). The organic layer was dried over anhydrous sodium sulfate and concentrated under vacuum to obtain the crude product. The residue was purified by flash silica gel chromatography (hexanes to hexanes/EtOAc=50/50, TLC: hexanes/EtOAc=1/1, R_{f}=0.4) to yield 1-(2-(benzyloxy)ethyl)-5-(4-(3-fluoro-5-(trifluoromethyl) phenoxy)pyridin-2-yl)-3-methyl-1,5-dihydro-4H-pyrazolo[4,3-c]pyridin-4-one (180 mg, 308.49 μmol, 87.40% yield) as colorless oil. LCMS [M+H]⁺=539.2; ¹H NMR (400 MHz, CDCl₃) δ=8.50 (d, J=6.0 Hz, 1H), 7.80 (d, J=7.6 Hz, 1H), 7.69 (d, J=2.4 Hz, 1H), 7.34-7.29 (m, 3H), 7.25 (br s, 2H), 7.22-7.17 (m, 2H), 7.10 (dd, J=2.0, 9.2 Hz, 1H), 6.90 (dd, J=2.0, 5.6 Hz, 1H), 6.51 (d, J=8.0 Hz, 1H), 4.48 (s, 2H), 4.37 (t, J=5.2 Hz, 2H), 3.88 (t, J=5.2 Hz, 2H), 2.65 (s, 3H).

Step B: To a solution of 1-(2-(benzyloxy)ethyl)-5-(4-(3-fluoro-5-(trifluoromethyl) phenoxy)pyridin-2-yl)-3-methyl-1,5-dihydro-4H-pyrazolo[4,3-c]pyridin-4-one (170 mg, 315.70 μmol, 1 eq.) in TFA (13.05 g, 114.43 mmol, 8.50 mL, 362.46 eq.). The mixture was stirred at 60° C. for 16 h. The reaction mixture was concentrated under reduced pressure to remove TFA. The residue was purified by prep-HPLC (column: 55-Boston Prime C18 150×30 mm, 5 μm; mobile phase: [H₂O (0.05% NH₃H₂O+10 mM NH₄HCO₃)-ACN]; gradient: 37%-67% ACN over 10.0 min) to yield 5-(4-(3-fluoro-5-(trifluoromethyl) phenoxy)pyridin-2-yl)-1-(2-hydroxyethyl)-3-methyl-1,5-dihydro-4H-pyrazolo[4,3-c]pyridin-4-one (59.32 mg, 131.59 μmol, 41.68% yield) as a white solid. LCMS [M+H]⁺=449.2; ¹H NMR (400 MHz, DMSO-d₆) δ=8.53 (d, J=5.6 Hz, 1H), 7.74 (d, J=7.6 Hz, 1H), 7.67 (br d, J=8.4 Hz, 1H), 7.59 (td, J=2.0, 10.0 Hz, 1H), 7.54 (s, 1H), 7.50 (d, J=2.4 Hz, 1H), 7.14 (dd, J=2.4, 6.0 Hz, 1H), 6.73 (d, J=7.6 Hz, 1H), 4.87 (t, J=5.2 Hz, 1H), 4.24 (t, J=5.2 Hz, 2H), 3.74 (q, J=5.6 Hz, 2H), 2.46 (s, 3H); ¹⁹F NMR (376 MHz, DMSO-d₆) δ=−61.269, −106.831.

### Example 40. Synthesis of 1-ethyl-6-(4-(3-fluoro-5-(trifluoromethyl) phenoxy)pyridin-2-yl)-3-(hydroxymethyl)-1,6-dihydro-7H-pyrazolo[3,4-c]pyridin-7-one (Compound 131)

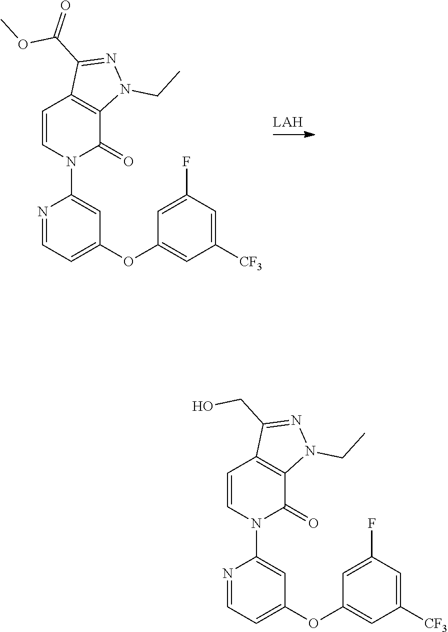 [SMILES: CCn1nc(C(=O)OC)c2ccn(-c3cc(Oc4cc(F)cc(C(F)(F)F)c4)ccn3)c(=O)c21 ; CCn1nc(CO)c2ccn(-c3cc(Oc4cc(F)cc(C(F)(F)F)c4)ccn3)c(=O)c21]

Step A: To a solution of methyl 1-ethyl-6-(4-(3-fluoro-5-(trifluoromethyl) phenoxy)pyridin-2-yl)-7-oxo-6,7-dihydro-1H-pyrazolo[3,4-c]pyridine-3-carboxylate (220 mg, 461.82 μmol, 1 eq.) in THF (10 mL) was added LiAlH₄ (2.5 M, 461.82 μL, 2.5 eq.). The mixture was stirred at 0° C. for 1 h under N₂ atmosphere. The reaction mixture was quenched by addition H₂O (20 mL) and extracted with EtOAc (30 mL×3). The combined organic layers were washed with brine (30 mL), dried over Na₂SO₄, filtered and concentrated under reduced pressure to give a residue, which was purified by prep-HPLC (column: Welch Xtimate C18 150*25 mm*5 μm; mobile phase: [H₂O (10 mM NH₄HCO₃)-ACN]; gradient: 43%-63% ACN over 11.0 min) to yield 1-ethyl-6-(4-(3-fluoro-5-(trifluoromethyl) phenoxy)pyridin-2-yl)-3-(hydroxymethyl)-1,6-dihydro-7H-pyrazolo[3,4-c]pyridin-7-one (36.4 mg, 78.78 μmol, 17.06% yield) as a white solid. LCMS [M+H]⁺=449.2; ¹H NMR (400 MHz, DMSO-d₆) δ=8.56 (d, J=5.6 Hz, 1H), 7.67 (br d, J=8.8 Hz, 1H), 7.64-7.59 (m, 1H), 7.58-7.52 (m, 2H), 7.47 (d, J=7.2 Hz, 1H), 7.17 (dd, J=2.4, 6.0 Hz, 1H), 6.78 (d, J=7.2 Hz, 1H), 5.33 (t, J=5.6 Hz, 1H), 4.67 (d, J=5.6 Hz, 2H), 4.62 (q, J=7.2 Hz, 2H), 1.36 (t, J=7.2 Hz, 3H); ¹⁹F NMR (376 MHz, DMSO-d₆) δ=−61.269, −106.843.

### Example 41. Synthesis of 6-(4-(3-(difluoromethyl)-5-fluorobenzyl)pyridin-2-yl)-4-(hydroxymethyl)-1,3-dimethyl-1,6-dihydro-7H-pyrazolo[3,4-d]pyridazin-7-one (Compound 136)

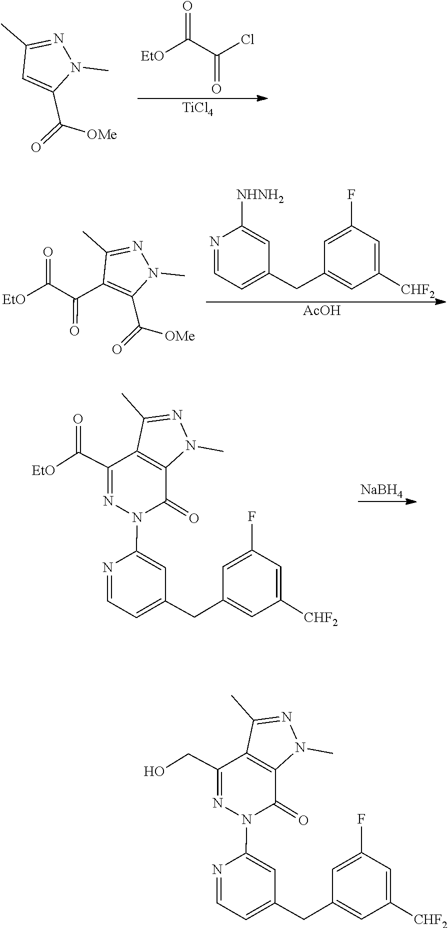

Step A: To a solution of TiCl₄ (11.07 g, 58.38 mmol, 3 eq.) in DCE (60 mL) was added ethyl 2-chloro-2-oxoacetate (7.97 g, 58.38 mmol, 6.52 mL, 3 eq.) at 0° C. The mixture was stirred at 25° C. for 0.5 h. Then, methyl 1,3-dimethyl-1H-pyrazole-5-carboxylate (3 g, 19.46 mmol, 1 eq.) was added to the reaction mixture at 25° C. The mixture was stirred at 50° C. for 2 h. The reaction was poured into ice water (50 mL). The solution was extracted DCM (50 mL×2). The organic layers were combined, washed with sat. NaHCO₃ (50 mL×2) and brine (50 mL). The mixture was dried over Na₂SO₄ and concentrated under vacuum to give a residue, which was purified by flash silica gel chromatography (hexane to hexane/EtOAc=80/20) to give methyl 4-(2-ethoxy-2-oxoacetyl)-1,3-dimethyl-1H-pyrazole-5-carboxylate (1.1 g, 4.33 mmol, 22.23% yield) as yellow oil: ¹H NMR (400 MHz, CDCl₃) δ=4.33 (q, J=7.2 Hz, 2H), 4.08 (s, 3H), 3.85 (s, 3H), 2.40 (s, 3H), 1.38 (t, J=7.2 Hz, 3H).

Step B: To a solution of methyl 4-(2-ethoxy-2-oxoacetyl)-1,3-dimethyl-1H-pyrazole-5-carboxylate (200 mg, 786.66 μmol, 1 eq.) in n-BuOH (5 mL) was added 4-(3-(difluoromethyl)-5-fluorobenzyl)-2-hydrazineylpyridine (420.47 mg, 1.57 mmol, 2 eq.) and HOAc (4.72 mg, 78.67 μmol, 4.50 μL, 0.1 eq.). The mixture was stirred at 140° C. for 2 h. The reaction mixture was concentrated under reduced pressure to remove solvent. The residue was diluted with water (10 mL) and extracted with EtOAc (20 mL×3). The combined organic layers were washed with brine (20 mL×2), dried over Na₂SO₄ and filtered. The filtrate was concentrated under reduced pressure to give a residue, which was purified by flash silica gel chromatography (hexane to hexane/EtOAc=60/40) to give ethyl 6-(4-(3-(difluoromethyl)-5-fluorobenzyl)pyridin-2-yl)-1,3-dimethyl-7-oxo-6,7-dihydro-1H-pyrazolo[3,4-d]pyridazine-4-carboxylate (100 mg, 137.88 μmol, 17.53% yield) as yellow oil. LCMS [M+H]⁺=472.0.

Step C: To a solution of ethyl 6-(4-(3-(difluoromethyl)-5-fluorobenzyl)pyridin-2-yl)-1,3-dimethyl-7-oxo-6,7-dihydro-1H-pyrazolo[3,4-d]pyridazine-4-carboxylate (80 mg, 169.70 μmol, 1 eq.) in MeOH (2 mL) was added NaBH₄ (40 mg, 1.06 mmol, 6.23 eq.) slowly under N₂ atmosphere at 0° C. The resulting mixture was stirred at 0° C. for 2 h under N₂ atmosphere. Water (10 mL) was added dropwise into the mixture to quench the reaction, and the mixture was extracted with EtOAc (10 mL×2). The combined organic layer was washed with brine (20 mL), dried over anhydrous sodium sulfate and filtered. The filtrate was concentrated under vacuum to give a residue, which was then purified by prep-HPLC [Column: Phenomenex Synergi C18 150*30 mm*4 μm, Condition: 43-63% (A: H₂O (0.225% FA), B: CH₃CN), flow rate: 25 mL/min] to give 6-(4-(3-(difluoromethyl)-5-fluorobenzyl)pyridin-2-yl)-4-(hydroxymethyl)-1,3-dimethyl-1,6-dihydro-7H-pyrazolo[3,4-d]pyridazin-7-one (33.98 mg, 78.53 μmol, 46.27% yield) as a white solid. LCMS [M+H]⁺=430.2; ¹H NMR (400 MHz, DMSO-d₆) δ=8.52 (d, J=4.8 Hz, 1H), 7.51 (s, 1H), 7.47-7.38 (m, 3H), 7.31 (d, J=8.8 Hz, 1H), 7.18-6.86 (m, 1H), 5.57 (t, J=5.6 Hz, 1H), 4.58 (d, J=5.6 Hz, 2H), 4.21 (s, 3H), 4.17 (s, 2H), 2.58 (s, 3H); ¹⁹F NMR (376 MHz, DMSO-d₆) δ=−110.350, −111.656.

### Example 42. Synthesis of 7-(4-(3-fluoro-5-(trifluoromethyl)benzyl)pyridin-2-yl)-3-methyl-1-(methylamino)-6,7-dihydroimidazo[1,5-a]pyrazin-8 (5H)-one (Compound 142)

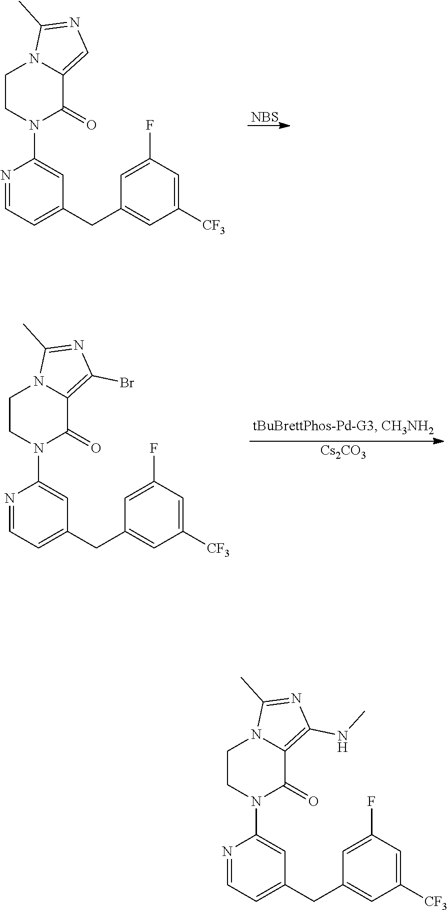 [SMILES: Cc1ncc2n1CCN(c1cc(Cc3cc(F)cc(C(F)(F)F)c3)ccn1)C2=O ; "NBS" ; Cc1nc(Br)c2n1CCN(c1cc(Cc3cc(F)cc(C(F)(F)F)c3)ccn1)C2=O ; "Cs2CO3" ; CNc1nc(C)n2c1C(=O)N(c1cc(Cc3cc(F)cc(C(F)(F)F)c3)ccn1)CC2]

Step A: To a solution of 7-(4-(3-fluoro-5-(trifluoromethyl)benzyl)pyridin-2-yl)-3-methyl-6,7-dihydroimidazo[1,5-a]pyrazin-8(5H)-one (166 mg, 410.53 μmol, 1 eq.) in MeCN (5 mL) was added NBS (109.60 mg, 615.79 μmol, 1.5 eq.) at 20° C. Then the reaction mixture was stirred at 70° C. for 2 h. The reaction mixture was diluted with water (5 mL) and extracted with EtOAc (5 mL×3). The combined organic layers were dried over Na₂SO₄, filtered and concentrated under reduced pressure to give a residue. The residue was purified by reversed-phase silica gel column (H₂O to H₂O/MeCN=50/50) to yield 1-bromo-7-(4-(3-fluoro-5-(trifluoromethyl)benzyl)pyridin-2-yl)-3-methyl-6,7-dihydroimidazo[1,5-a]pyrazin-8(5H)-one (65 mg, 114.33 μmol, 27.85% yield) as a white solid. LCMS [M+H]⁺=483.0; ¹H NMR (400 MHz, DMSO-d₆) δ=8.37 (d, J=4.8 Hz, 1H), 7.86 (s, 1H), 7.60 (s, 1H), 7.58-7.51 (m, 2H), 7.16 (dd, J=1.2, 5.2 Hz, 1H), 4.38-4.31 (m, 2H), 4.28-4.23 (m, 2H), 4.15 (s, 2H), 2.36 (s, 3H).

Step B: To a solution of 1-bromo-7-(4-(3-fluoro-5-(trifluoromethyl)benzyl)pyridin-2-yl)-3-methyl-6,7-dihydroimidazo[1,5-a]pyrazin-8(5H)-one (60 mg, 124.16 μmol, 1 eq.) and methylamine hydrochloride (41.91 mg, 620.79 μmol, 5 eq.) in DMF (2 mL) was added BuBrettPhos-Pd-G3 (10.61 mg, 12.42 μmol, 0.1 eq.) and Cs₂CO₃ (323.62 mg, 993.26 μmol, 8 eq.) at 20° C. under N₂ atmosphere. Then the reaction mixture was stirred at 100° C. for 4 h under N₂ atmosphere. The reaction mixture was cooled and filtered then concentrated under reduced pressure to give a residue. The residue was purified by prep-HPLC (column: Welch Xtimate C18 150*25 mm*5 μm; mobile phase: [H₂O (10 mM NH₄HCO₃)-ACN]; gradient: 35%-65% ACN over 11.0 min) to yield 7-(4-(3-fluoro-5-(trifluoromethyl)benzyl)pyridin-2-yl)-3-methyl-1-(methylamino)-6,7-dihydroimidazo[1,5-a]pyrazin-8(5H)-one (30 mg, 67.85 μmol, 54.65% yield) as a white solid. LCMS [M+H]⁺=434.1; ¹H NMR (400 MHz, DMSO-d₆) δ=8.29 (d, J=5.2 Hz, 1H), 7.81 (s, 1H), 7.55 (br s, 2H), 7.48 (br d, J=9.2 Hz, 1H), 7.08-6.98 (m, 1H), 5.71 (q, J=4.8 Hz, 1H), 4.29-4.18 (m, 2H), 4.14-4.05 (m, 4H), 2.86 (d, J=5.2 Hz, 3H), 2.25 (s, 3H).

### Example 43. Synthesis of 3-amino-5-(4-(3-fluoro-5-(trifluoromethyl)benzyl)pyridin-2-yl)-1-methyl-1,5-dihydro-4H-pyrazolo[4,3-c]pyridin-4-one (Compound 153)

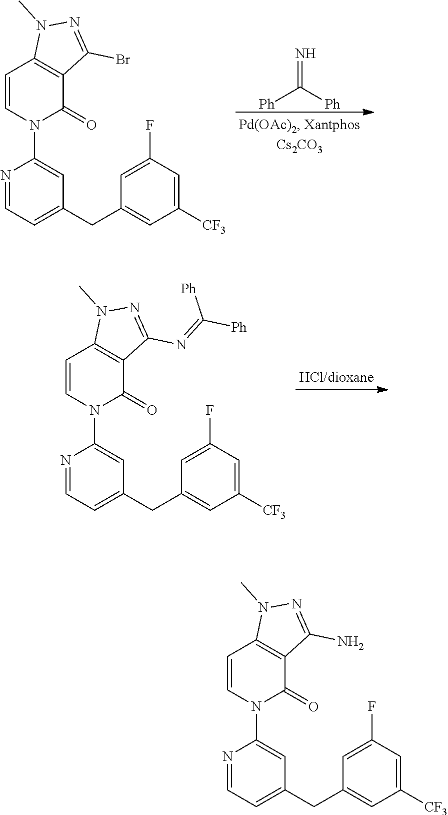 [SMILES: Cn1nc(Br)c2c(=O)n(-c3cc(Cc4cc(F)cc(C(F)(F)F)c4)ccn3)ccc21 ; N=C(c1ccccc1)c1ccccc1 ; Cn1nc(N=C(c2ccccc2)c2ccccc2)c2c(=O)n(-c3cc(Cc4cc(F)cc(C(F)(F)F)c4)ccn3)ccc21 ; Cn1nc(N)c2c(=O)n(-c3cc(Cc4cc(F)cc(C(F)(F)F)c4)ccn3)ccc21]

Step A: To a solution of 3-bromo-5-(4-(3-fluoro-5-(trifluoromethyl)benzyl)pyridin-2-yl)-1-methyl-1,5-dihydro-4H-pyrazolo[4,3-c]pyridin-4-one (300 mg, 623.39 μmol, 1 eq.) and benzophenone imine (112.98 mg, 623.39 μmol, 104.61 μL, 1 eq.) in dioxane (10 mL) was added Cs₂CO₃ (609.34 mg, 1.87 mmol, 3 eq.), Pd(OAc)₂ (27.99 mg, 124.68 μmol, 0.2 eq.) and Xantphos (4,5-bis(diphenylphosphino)-9,9-dimethylxanthene, 36.07 mg, 62.34 μmol, 0.1 eq.) at 25° C. Then the mixture was stirred at 90° C. for 12 h under N₂. The mixture was extracted with DCM (100 mL). The combined organic layers were dried over Na₂SO₄, filtered and concentrated to give a residue, which was purified by flash silica gel chromatography (hexane to hexane/EtOAc=1/4, TLC: hexane/EtOAc=1/3, R_{f}=0.6) to give 3-((diphenylmethylene)amino)-5-(4-(3-fluoro-5-(trifluoromethyl)benzyl)pyridin-2-yl)-1-methyl-1,5-dihydro-4H-pyrazolo[4,3-c]pyridin-4-one (210 mg, 361.10 μmol, 57.92% yield) as a yellow solid: ¹H NMR (400 MHz, DMSO-d₆) δ=8.45 (d, J=5.2 Hz, 1H), 7.70-7.46 (m, 10H), 7.34 (d, J=4.0 Hz, 1H), 7.31-7.22 (m, 3H), 7.21-7.14 (m, 2H), 6.60 (d, J=7.6 Hz, 1H), 4.18 (s, 2H), 3.77 (s, 3H).

Step B: To a solution of 3-((diphenylmethylene)amino)-5-(4-(3-fluoro-5-(trifluoromethyl)benzyl)pyridin-2-yl)-1-methyl-1,5-dihydro-4H-pyrazolo[4,3-c]pyridin-4-one (200 mg, 343.90 μmol, 1 eq.) in DCM (3 mL) was added HCl/dioxane (2 M, 1.72 mL, 10 eq.). The mixture was stirred at 20° C. for 0.5 h. Then the mixture was neutralized by sat. aq. NaHCO₃ (80 mL) and extracted with (100 mL×2). The combined organic layer was dried over Na₂SO₄, filtered and concentrated to give a residue, which was purified by flash silica gel chromatography (EtOAc to EtOAc/MeOH=10/1, TLC: EtOAc/MeOH=10/1, R_{f}=0.6) to give 3-amino-5-(4-(3-fluoro-5-(trifluoromethyl)benzyl)pyridin-2-yl)-1-methyl-1,5-dihydro-4H-pyrazolo[4,3-c]pyridin-4-one (116 mg, 277.19 μmol, 80.60% yield) as a white solid. LCMS [M+H]⁺=418.1; ¹H NMR (400 MHz, DMSO-d₆) δ=8.48 (d, J=4.8 Hz, 1H), 7.75 (s, 1H), 7.68-7.59 (m, 2H), 7.55 (d, J=9.2 Hz, 2H), 7.36 (d, J=4.4 Hz, 1H), 6.59 (d, J=7.2 Hz, 1H), 5.42 (s, 2H), 4.18 (s, 2H), 3.68 (s, 3H).

### Example 44. Synthesis of 6-(4-(3-fluoro-5-(trifluoromethyl)benzyl)pyridin-2-yl)-3-(3-hydroxyazetidin-1-yl)-1-methyl-1,4,5,6-tetrahydro-7H-pyrazolo[3,4-c]pyridin-7-one (Compound 174)

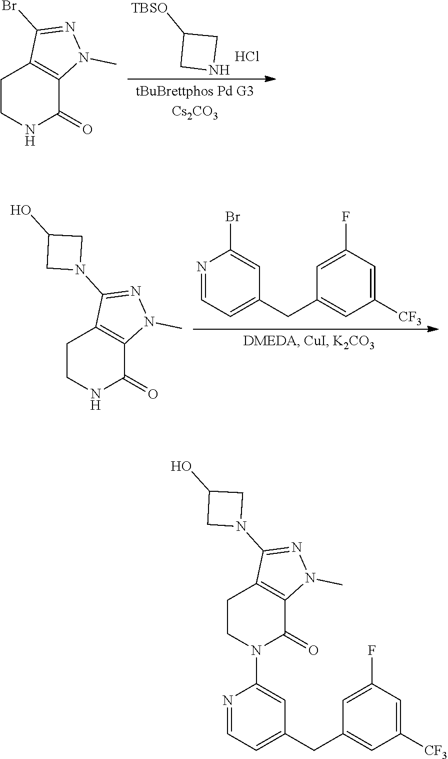

Step A: A mixture of 3-bromo-1-methyl-1,4,5,6-tetrahydro-7H-pyrazolo[3,4-c]pyridin-7-one (100 mg, 434.67 μmol, 1 eq.), 3-((tert-butyldimethylsilyl)oxy) azetidine hydrochloride (145.93 mg, 652.00 μmol, 1.5 eq, HCl), Cs₂CO₃ (424.87 mg, 1.30 mmol, 3 eq.), tert-BuBrettPhos-Pd-G3 (37.14 mg, 43.47 μmol, 0.1 eq.) in DMF (1 mL) was degassed and purged with N₂ three times. Then, the mixture was stirred at 90° C. for 16 h under N₂ atmosphere. The reaction mixture was cooled and diluted with EtOAc (5 mL), washed by water (2 mL) and brine (2 mL). The organic layer was dried over anhydrous sodium sulfate and concentrated under vacuum. The residue was purified by prep-HPLC (column: 40-WePure Biotech XP tC18 150×30 mm, 7 μm; mobile phase: [H₂O (0.05% NH₃H₂O+10 mM NH₄HCO₃)-ACN]; gradient: 42%-72% ACN over 10.0 min) to yield 3-(3-hydroxyazetidin-1-yl)-1-methyl-1,4,5,6-tetrahydro-7H-pyrazolo[3,4-c]pyridin-7-one (120 mg, 431.96 μmol, 99.38% yield) as a white solid. LCMS [M+H]⁺=223.1; ¹H NMR (400 MHz, DMSO-d₆) δ=7.71 (br s, 1H), 4.53-4.45 (m, 1H), 4.05-3.99 (m, 2H), 3.86 (s, 3H), 3.58-3.51 (m, 2H), 3.34-3.28 (m, 3H), 3.17 (s, 2H).

Step B: A mixture of 3-(3-hydroxyazetidin-1-yl)-1-methyl-1,4,5,6-tetrahydro-7H-pyrazolo[3,4-c]pyridin-7-one (60 mg, 269.97 μmol, 1 eq.), 2-bromo-4-(3-fluoro-5-(trifluoromethyl)benzyl)pyridine (90.20 mg, 269.97 μmol, 1 eq.), CuI (10.28 mg, 53.99 μmol, 0.2 eq.), DMEDA (9.52 mg, 107.99 μmol, 11.62 μL, 0.4 eq.) and K₂CO₃ (149.25 mg, 1.08 mmol, 4 eq.) in dioxane (1 mL) was degassed and purged with N₂ three times. Then the mixture was stirred at 110° C. for 16 h under N₂ atmosphere. The reaction mixture was diluted with EtOAc (5 mL), washed by water (3 mL) and brine (3 mL). The organic layer was dried over anhydrous sodium sulfate and concentrated under vacuum. The residue was purified by prep-HPLC column: 40-WePure Biotech XP tC18 150×30 mm, 7 μm; mobile phase: [H₂O (0.05% NH₃H₂O+10 mM NH₄HCO₃)-ACN]; gradient: 37%-67% ACN over 10.0 min) to yield 6-(4-(3-fluoro-5-(trifluoromethyl)benzyl)pyridin-2-yl)-3-(3-hydroxyazetidin-1-yl)-1-methyl-1,4,5,6-tetrahydro-7H-pyrazolo[3,4-c]pyridin-7-one (40.62 mg, 85.44 μmol, 31.65% yield) as a white solid. LCMS [M+H]⁺=476.1; ¹H NMR (400 MHz, DMSO-d₆) δ=8.36 (d, J=5.2 Hz, 1H), 7.68 (s, 1H), 7.59 (s, 1H), 7.57-7.48 (m, 2H), 7.15 (d, J=4.4 Hz, 1H), 5.58 (d, J=6.8 Hz, 1H), 4.55-4.44 (m, 1H), 4.17-4.10 (m, 4H), 4.06-4.01 (m, 2H), 3.91 (s, 3H), 3.60-3.54 (m, 2H), 2.77-2.68 (m, 2H).

### Example 45. Synthesis of 5-(4-(3-fluoro-5-(trifluoromethyl) phenoxy)pyridin-2-yl)-1-(2-hydroxyethyl)-3-methyl-1,5-dihydro-4H-pyrazolo[3,4-d]pyrimidin-4-one (Compound 182)

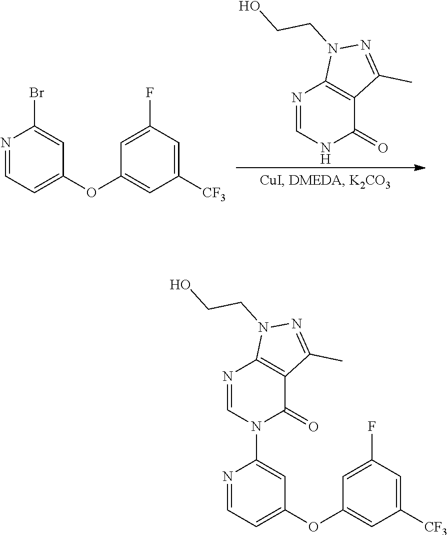 [SMILES: Fc1cc(Oc2ccnc(Br)c2)cc(C(F)(F)F)c1 ; Cc1nn(CCO)c2nc[nH]c(=O)c12 ; Cc1nn(CCO)c2ncn(-c3cc(Oc4cc(F)cc(C(F)(F)F)c4)ccn3)c(=O)c12]

Step A: To a solution of 1-(2-hydroxyethyl)-3-methyl-1,5-dihydro-4H-pyrazolo[3,4-d]pyrimidin-4-one (50 mg, 257.48 μmol, 1 eq.) and 2-bromo-4-(3-fluoro-5-(trifluoromethyl) phenoxy)pyridine (103.84 mg, 308.98 μmol, 1.2 eq.) in dioxane (3 mL) was added CuI (9.81 mg, 51.50 μmol, 0.2 eq.), DMEDA (9.08 mg, 102.99 μmol, 11.09 μL, 0.4 eq.) and K₂CO₃ (106.76 mg, 772.44 μmol, 3 eq.), the mixture was stirred at 110° C. for 2 h under N₂ atmosphere. The reaction mixture was filtrated and concentrated under vacuum. Then, the residue was purified by prep-HPLC (column: 40-WePure Biotech XP tC18 150×30 mm, 7 μm; mobile phase: [H₂O (0.05% NH₃H₂O+10 mM NH₄HCO₃)-ACN]; gradient: 32%-62% ACN over 10.0 min) to yield 5-(4-(3-fluoro-5-(trifluoromethyl) phenoxy)pyridin-2-yl)-1-(2-hydroxyethyl)-3-methyl-1,5-dihydro-4H-pyrazolo[3,4-d]pyrimidin-4-one (44.43 mg, 96.14 μmol, 37.34% yield) as a white solid. LCMS [M+H]⁺=450.1; ¹H NMR (400 MHz, DMSO-d₆) δ=8.58 (d, J=6.0 Hz, 1H), 8.46 (s, 1H), 7.68 (br d, J=8.4 Hz, 1H), 7.60 (br d, J=9.6 Hz, 1H), 7.55 (s, 1H), 7.47 (d, J=2.0 Hz, 1H), 7.25 (dd, J=2.4, 6.0 Hz, 1H), 4.89 (br s, 1H), 4.27 (t, J=6.0 Hz, 2H), 3.79 (br d, J=4.4 Hz, 2H), 2.44 (s, 3H).

### Example 46. Synthesis of 7-(4-(3-fluoro-5-(trifluoromethyl) phenoxy)pyridin-2-yl)-3-(2-hydroxyethyl)-1-methyl-6,7-dihydroimidazo[1,5-a]pyrazin-8(5H)-one (Compound 195)

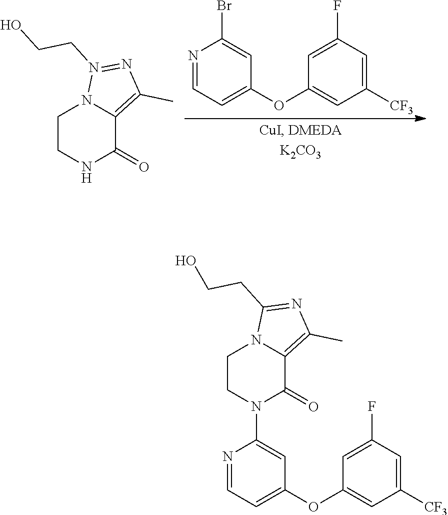

Step A: To a solution of 3-(2-hydroxyethyl)-1-methyl-6,7-dihydroimidazo[1,5-a]pyrazin-8(5H)-one (100.00 mg, 512.25 μmol, 1.0 eq.) and 2-bromo-4-(3-fluoro-5-(trifluoromethyl) phenoxy)pyridine (37.72 mg, 409.80 μmol, 0.8 eq.) in dioxane (2 mL) was added K₂CO₃ (12.39 mg, 1.54 mmol, 3 eq.), CuI (19.51 mg, 102.45 μmol, 0.2 eq.) and DMEDA (18.06 mg, 204.90 μmol, 22.05 μL, 0.4 eq.) at 20° C. under N₂ atmosphere. Then the reaction mixture was stirred at 110° C. for 3 h under N₂ atmosphere. The reaction mixture was quenched diluted with H₂O (10 mL) and extracted with EtOAc (10 mL×3). The combined organic layers were dried over Na₂SO₄, filtered and concentrated under reduced pressure to give a residue. The residue was purified by prep-HPLC (column: 41-WePure Biotech XP tC18 150×40 mm, 7 μm; mobile phase: [H₂O (10 mM NH₄HCO₃)-ACN]; gradient: 40%-70% B over 11.0 min) to yield 7-(4-(3-fluoro-5-(trifluoromethyl) phenoxy)pyridin-2-yl)-3-(2-hydroxyethyl)-1-methyl-6,7-dihydroimidazo[1,5-a]pyrazin-8(5H)-one (93 mg, 203.72 μmol, 39.77% yield) as a white solid. LCMS [M+H]⁺=451.3; ¹H NMR (400 MHz, DMSO-d₆) δ=8.39 (d, J=6.0 Hz, 1H), 7.68-7.58 (m, 2H), 7.54 (br d, J=10.0 Hz, 1H), 7.48 (s, 1H), 6.91 (dd, J=2.4, 5.6 Hz, 1H), 4.82 (t, J=5.6 Hz, 1H), 4.38-4.30 (m, 2H), 4.26-4.19 (m, 2H), 3.74-3.67 (m, 2H), 2.80 (t, J=6.4 Hz, 2H), 2.35 (s, 3H).

### Example 47. Synthesis of 7-(4-(3-fluoro-5-(trifluoromethyl)benzyl)pyridin-2-yl)-3-(hydroxymethyl)-1-(methylamino)-6,7-dihydroimidazo[1,5-a]pyrazin-8(5H)-one (Compound 203)

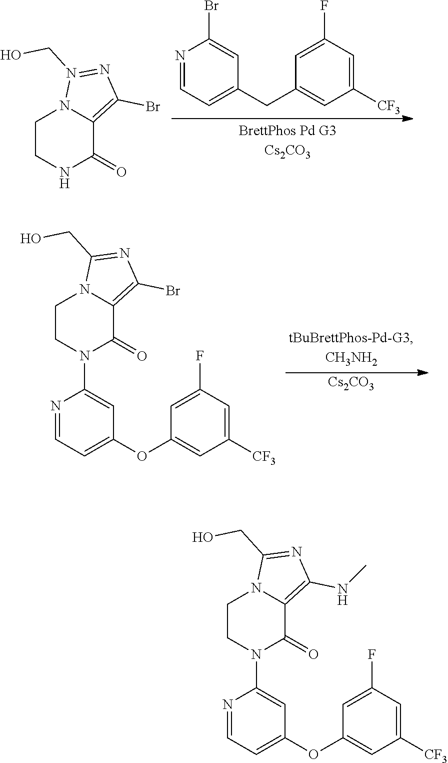

Step A: To a solution of 1-bromo-3-(hydroxymethyl)-6,7-dihydroimidazo[1,5-a]pyrazin-8(5H)-one (85 mg, 345.44 μmol, 1 eq.) and 2-bromo-4-(3-fluoro-5-(trifluoromethyl)benzyl)pyridine (92.33 mg, 276.35 μmol, 0.8 eq.) in dioxane (6 mL) was added Cs₂CO₃ (337.66 mg, 1.04 mmol, 3 eq.) and BrettPhos Pd G3 ([(2-di-cyclohexylphosphino-3,6-dimethoxy-2′,4′,6′-triisopropyl-1,1′-biphenyl)-2-(2′-amino-1,1′-biphenyl)]palladium (II) methanesulfonate, CAS #1470372-59-8, 31.31 mg, 34.54 μmol, 0.1 eq.) at 20° C. under N₂ atmosphere. Then the reaction mixture was stirred at 90° C. for 4 h under N₂ atmosphere. The reaction mixture was filtered and concentrated under reduced pressure to give a residue. The residue was purified by flash silica gel chromatography (DCM to DCM/MeOH=93/7) to yield 1-bromo-7-(4-(3-fluoro-5-(trifluoromethyl)benzyl)pyridin-2-yl)-3-(hydroxymethyl)-6,7-dihydroimidazo[1,5-a]pyrazin-8(5H)-one (80 mg, 112.17 μmol, 32.47% yield) as a yellow solid: ¹H NMR (400 MHz, DMSO-d₆) δ=8.37 (d, J=4.0 Hz, 1H), 7.87 (s, 1H), 7.60-7.53 (m, 3H), 7.17 (s, 1H), 5.60 (t, J=4.8 Hz, 1H), 4.57 (br d, J=4.4 Hz, 2H), 4.40-4.32 (m, 4H), 4.15 (s, 2H).

Step B: To a solution of 1-bromo-7-(4-(3-fluoro-5-(trifluoromethyl)benzyl)pyridin-2-yl)-3-(hydroxymethyl)-6,7-dihydroimidazo[1,5-a]pyrazin-8(5H)-one (70 mg, 140.21 μmol, 1 eq.) and methylamine hydrochloride (47.33 mg, 701.04 μmol, 5 eq.) in DMF (3 mL) was added Cs₂CO₃ (365.46 mg, 1.12 mmol, 8 eq.) and/BuBrettPhos Pd G3 ([(2-di-tert-butylphosphino-3,6-dimethoxy-2′,4′,6′-triisopropyl-1,1′-biphenyl)-2-(2′-amino-1,1′-biphenyl)]palladium (II) methanesulfonate, CAS #1536473-72-9, 11.98 mg, 14.02 μmol, 0.1 eq.) at 20° C. under N₂ atmosphere. Then the reaction mixture was stirred at 90° C. for 16 h under N₂ atmosphere. Then, the reaction mixture was diluted with H₂O (10 mL) and extracted with EtOAc (10 mL×3). The combined organic layers were dried over Na₂SO₄, filtered and concentrated under reduced pressure to give a residue. The residue was purified by prep-HPLC (column: Welch Xtimate C18 150*25 mm*5 μm; mobile phase: [H₂O (10 mM NH₄HCO₃)-ACN]; gradient: 30%-60% ACN over 11.0 min) to yield 7-(4-(3-fluoro-5-(trifluoromethyl)benzyl)pyridin-2-yl)-3-(hydroxymethyl)-1-(methylamino)-6,7-dihydroimidazo[1,5-a]pyrazin-8(5H)-one (25 mg, 54.98 μmol, 39.21% yield) as a white solid. LCMS [M+H]⁺=450.3; ¹H NMR (400 MHz, DMSO-d₆) δ=8.30 (d, J=5.2 Hz, 1H), 7.82 (s, 1H), 7.58-7.45 (m, 3H), 7.04 (br d, J=4.8 Hz, 1H), 5.74 (br d, J=5.2 Hz, 1H), 5.46 (t, J=5.6 Hz, 1H), 4.45 (d, J=6.0 Hz, 2H), 4.22 (br d, J=5.2 Hz, 4H), 4.10 (s, 2H), 2.86 (d, J=4.8 Hz, 3H).

### Example 48. Synthesis of 1-ethyl-7-(4-(3-fluoro-5-(trifluoromethyl) phenoxy)pyridin-2-yl)-3-(hydroxymethyl)-6,7-dihydroimidazo[1,5-a]pyrazin-8(5H)-one (Compound 210)

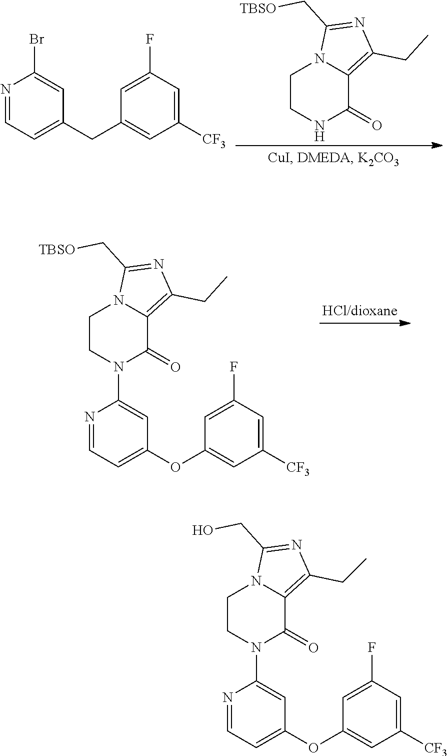 [SMILES: Fc1cc(Cc2ccnc(Br)c2)cc(C(F)(F)F)c1 ; CCc1nc(CO[Si](C)(C)C(C)(C)C)n2c1C(=O)NCC2 ; CCc1nc(CO[Si](C)(C)C(C)(C)C)n2c1C(=O)N(c1cc(Oc3cc(F)cc(C(F)(F)F)c3)ccn1)CC2 ; CCc1nc(CO)n2c1C(=O)N(c1cc(Oc3cc(F)cc(C(F)(F)F)c3)ccn1)CC2]

Step A: A mixture of 2-bromo-4-(3-fluoro-5-(trifluoromethyl) phenoxy)pyridine (70 mg, 208.28 μmol, 9.67e-1 eq.), 3-(((tert-butyldimethylsilyl)oxy)methyl)-1-ethyl-6,7-dihydroimidazo[1,5-a]pyrazin-8(5H)-one (80 mg, 258.50 μmol, 1.2 eq.), CuI (8.21 mg, 43.08 μmol, 0.2 eq.), DMEDA (7.60 mg, 86.17 μmol, 9.27 μL, 0.4 eq.) and K₂CO₃ (119.09 mg, 861.67 μmol, 4 eq.) in dioxane (1 mL) was degassed and purged with N₂ three times, and then the mixture was stirred at 110° C. for 5 h under N₂ atmosphere. Then, the reaction mixture was diluted with EtOAc (10 mL×3), washed by water (10 mL) and brine (10 mL). The organic layer was dried over anhydrous sodium sulfate and concentrated under vacuum. The residue was purified by flash silica gel chromatography (hexane to hexane/EtOAc=76/24) to yield 3-(((tert-butyldimethylsilyl)oxy)methyl)-1-ethyl-7-(4-(3-fluoro-5-(trifluoromethyl) phenoxy)pyridin-2-yl)-6,7-dihydroimidazo[1,5-a]pyrazin-8(5H)-one (110 mg, 175.33 μmol, 81.39% yield) as a white solid. LCMS [M+H]⁺=565.3; ¹H NMR (400 MHz, DMSO-d₆) δ=8.40 (d, J=5.6 Hz, 1H), 7.65 (br d, J-8.4 Hz, 1H), 7.61 (d, J=2.0 Hz, 1 H), 7.51-7.57 (m, 1H), 7.49 (s, 1H), 6.93 (dd, J-5.6, 2.0 Hz, 1H), 4.75 (s, 2H), 4.25-4.42 (m, 4H), 2.73-2.83 (m, 2H), 1.11-1.16 (m, 3H), 0.86 (s, 9H), 0.06 (s, 6H).

Step B: To a solution of 3-(((tert-butyldimethylsilyl)oxy)methyl)-1-ethyl-7-(4-(3-fluoro-5-(trifluoromethyl) phenoxy)pyridin-2-yl)-6,7-dihydroimidazo[1,5-a]pyrazin-8 (5H)-one (100 mg, 177.10 μmol, 1 eq.) in DCM (1 mL) was added HCl/dioxane (2 M, 1 mL). The mixture was stirred at 25° C. for 1 h. Then, aq. NaHCO₃ was added to adjust the mixture to PH ~7. The reaction mixture was diluted with EtOAc (5 mL×3), washed by water (2 mL) and brine (2 mL). The organic layer was dried over anhydrous sodium sulfate and concentrated under vacuum. The residue was purified by prep-HPLC (column: 40-WePure Biotech XP tC18 150×30 mm, 7 μm; mobile phase: [H₂O (0.05% NH₃H₂O+10 mM NH₄HCO₃)-ACN]; gradient: 38%-68% ACN over 10.0 min) to yield 1-ethyl-7-(4-(3-fluoro-5-(trifluoromethyl) phenoxy)pyridin-2-yl)-3-(hydroxymethyl)-6,7-dihydroimidazo[1,5-a]pyrazin-8(5H)-one (64.5 mg, 143.21 μmol, 80.86% yield) as a white solid. LCMS [M+H]⁺=451.1; ¹H NMR (400 MHz, DMSO-d₆) δ=8.40 (d, J=5.6 Hz, 1H), 7.60-7.68 (m, 2H), 7.54 (br d, J=10.0 Hz, 1H), 7.48 (s, 1H), 6.86-6.95 (m, 1H), 5.48 (t, J=5.6 Hz, 1H), 4.53 (br d, J=5.6 Hz, 2H), 4.33 (br d, J=10.8 Hz, 4H), 2.76-2.84 (m, 2H), 1.14 (t, J=7.2 Hz, 3 H).

### Example 49. Synthesis of 7-(4-(3-cyclopropyl-5-fluorophenoxy)pyridin-2-yl)-3-(2-hydroxyethyl)-1-methyl-6,7-dihydroimidazo[1,5-a]pyrazin-8(5H)-one (Compound 222)

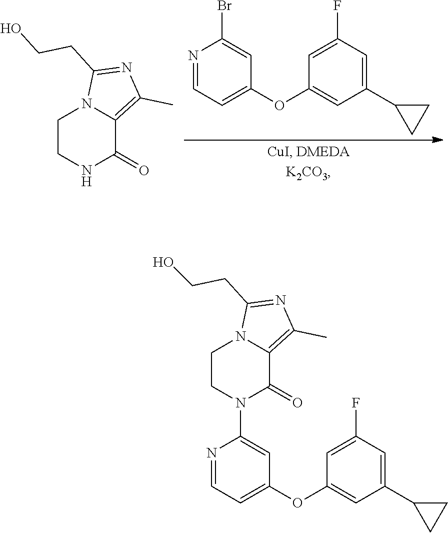

Step A: To a solution of 3-(2-hydroxyethyl)-1-methyl-6,7-dihydroimidazo[1,5-a]pyrazin-8(5H)-one (130 mg, 665.92 μmol, 1 eq.) and 2-bromo-4-(3-cyclopropyl-5-fluorophenoxy)pyridine (184.68 mg, 599.33 μmol, 0.9 eq.) in dioxane (5 mL) was added K₂CO₃ (276.10 mg, 2.00 mmol, 3 eq.), DMEDA (23.48 mg, 266.37 μmol, 28.67 μL, 0.4 eq.) and CuI (25.36 mg, 133.18 μmol, 0.2 eq.) at 20° C. under N₂ atmosphere. Then the reaction mixture was stirred at 110° C. for 16 h under N₂ atmosphere. Then, the reaction mixture was diluted with H₂O (10 mL) and extracted with EtOAc (10 mL×3). The combined organic layers were dried over Na₂SO₄, filtered and concentrated under reduced pressure to give a residue, which was purified by prep-HPLC (column: Welch Xtimate C18 150*25 mm*5 μm; mobile phase: [H₂O (10 mM NH₄HCO₃)-ACN]; gradient: 36%-66% ACN over 11.0 min]) to yield 7-(4-(3-cyclopropyl-5-fluorophenoxy)pyridin-2-yl)-3-(2-hydroxyethyl)-1-methyl-6,7-dihydroimidazo[1,5-a]pyrazin-8(5H)-one (120 mg, 284.06 μmol, 47.40% yield) as a white solid. LCMS [M+H]⁺=423.3; ¹H NMR (400 MHz, DMSO-d₆) δ=8.34 (d, J=5.6 Hz, 1H), 7.50 (d, J=2.4 Hz, 1H), 6.91-6.76 (m, 4H), 4.80 (br t, J=4.8 Hz, 1H), 4.36-4.27 (m, 2H), 4.25-4.17 (m, 2H), 3.75-3.65 (m, 2H), 2.80 (t, J=6.4 Hz, 2H), 2.35 (s, 3H), 2.03-1.93 (m, 1H), 1.04-0.94 (m, 2H), 0.82-0.72 (m, 2H).

### Example 50. Synthesis of 7-(4-(3-fluoro-5-(trifluoromethyl) phenoxy)pyridin-2-yl)-3-(2-hydroxyethoxy)-1-methyl-6,7-dihydroimidazo[1,5-a]pyrazin-8(5H)-one (Compound 233)

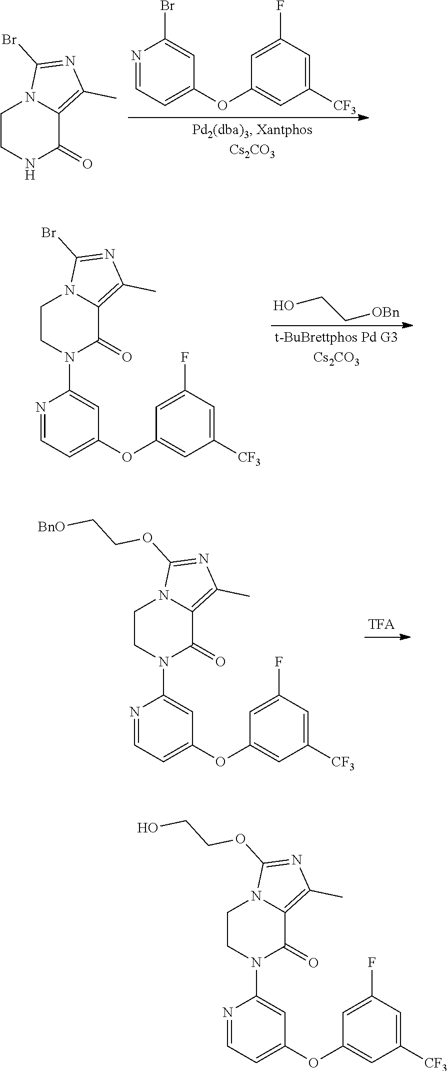

Step A: To a solution of 3-bromo-1-methyl-6,7-dihydroimidazo[1,5-a]pyrazin-8(5H)-one (200 mg, 869.33 μmol, 1 eq.) and 2-bromo-4-(3-fluoro-5-(trifluoromethyl) phenoxy)pyridine (292.16 mg, 869.33 μmol, 1 eq.) in dioxane (10 mL) was added Pd₂(dba)₃ (79.61 mg, 86.93 μmol, 0.1 eq.), Xantphos (50.30 mg, 86.93 μmol, 0.1 eq.) and Cs₂CO₃ (566.49 mg, 1.74 mmol, 2 eq.). The mixture was stirred at 90° C. for 16 h. Then, the reaction mixture was concentrated to give a residue. The residue was purified by flash silica gel chromatography (hexanes to hexanes/EtOAc=55/45, TLC: Hexane/EtOAc=1/1, R_{f}=0.4) to give 3-bromo-7-(4-(3-fluoro-5-(trifluoromethyl) phenoxy)pyridin-2-yl)-1-methyl-6,7-dihydroimidazo[1,5-a]pyrazin-8 (5/)-one (163 mg, 335.92 μmol, 38.64% yield) as a white solid. LCMS [M+H]⁺=484.9.

Step B: To a solution of 3-bromo-7-(4-(3-fluoro-5-(trifluoromethyl) phenoxy)pyridin-2-yl)-1-methyl-6,7-dihydroimidazo[1,5-a]pyrazin-8(5H)-one (150 mg, 309.13 μmol, 1 eq.) and 2-(benzyloxy) ethan-1-ol (141.14 mg, 927.40 μmol, 131.78 μL, 3 eq.) in DMF (10 mL) was added Cs₂CO₃ (302.16 mg, 927.40 μmol, 3 eq.) and t-BuBrettphos Pd G3 (26.41 mg, 30.91 μmol, 0.1 eq.). The mixture was stirred at 90° C. for 4 h. Then, the reaction mixture was concentrated to give a residue. The residue was purified by flash silica gel chromatography (hexanes to hexanes/EtOAc=80/20, TLC: hexanes/EtOAc=1/1, R_{f}=0.25) to give 3-(2-(benzyloxy) ethoxy)-7-(4-(3-fluoro-5-(trifluoromethyl) phenoxy)pyridin-2-yl)-1-methyl-6,7-dihydroimidazo[1,5-a]pyrazin-8(5H)-one (93 mg, 167.11 μmol, 54.06% yield) as colorless oil: ¹H NMR (400 MHz, CDCl₃)=8.33 (d, J=6.0 Hz, 1H), 7.74 (d, J=2.0 Hz, 1H), 7.39-7.29 (m, 5H), 7.22-7.16 (m, 2H), 7.05-6.98 (m, 1H), 6.70 (dd, J=2.0, 5.6 Hz, 1H), 4.66-4.56 (m, 4H), 4.45-4.37 (m, 2H), 4.08-3.99 (m, 2H), 3.87-3.81 (m, 2H), 2.50 (s, 3H).

Step C: 3-(2-(benzyloxy) ethoxy)-7-(4-(3-fluoro-5-(trifluoromethyl) phenoxy)pyridin-2-yl)-1-methyl-6,7-dihydroimidazo[1,5-a]pyrazin-8(5H)-one (80 mg, 143.75 μmol, 1 eq.) was dissolved in TFA (3 mL) and the mixture was stirred at 70° C. for 16 h. Then, the reaction mixture was concentrated to give a residue and adjusted pH=8 with aq. NaHCO₃. The mixture was purified by prep-HPLC (column: Welch Xtimate C18 150*25 mm*5 μm; mobile phase: [H₂O (10 mM NH₄HCO₃)-ACN]; gradient: 34%-64% ACN over 11.0 min) to give 7-(4-(3-fluoro-5-(trifluoromethyl) phenoxy)pyridin-2-yl)-3-(2-hydroxyethoxy)-1-methyl-6,7-dihydroimidazo[1,5-a]pyrazin-8(5H)-one (19.8 mg, 42.45 μmol, 29.53% yield) as a white solid. LCMS [M+H]⁺=467.2; ¹H NMR (400 MHz, DMSO-d₆)=8.38 (d, J=5.6 Hz, 1H), 7.65 (d, J=8.2 Hz, 1H), 7.60-7.51 (m, 2H), 7.48 (s, 1H), 6.90 (dd, J=2.4, 6.0 Hz, 1H), 4.94 (t, J=5.6 Hz, 1H), 4.41-4.26 (m, 4H), 4.06-3.93 (m, 2H), 3.76-3.62 (m, 2H), 2.33 (s, 3H).

### Example 51. Synthesis of 5-(4-(3-fluoro-5-(trifluoromethyl) phenoxy)pyridin-2-yl)-1-(2-hydroxyethyl)-3-methyl-3,5,6,7-tetrahydro-1H-imidazo[4,5-c]pyridine-2,4-dione (Compound 235)

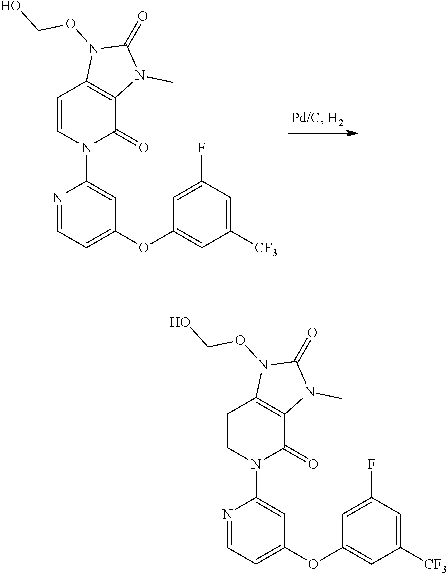 [SMILES: Cn1c(=O)n(OCO)c2ccn(-c3cc(Oc4cc(F)cc(C(F)(F)F)c4)ccn3)c(=O)c21 ; Cn1c2c(n(OCO)c1=O)CCN(c1cc(Oc3cc(F)cc(C(F)(F)F)c3)ccn1)C2=O]

Step A: To a solution of 5-(4-(3-fluoro-5-(trifluoromethyl) phenoxy)pyridin-2-yl)-1-(2-hydroxyethyl)-3-methyl-3,5-dihydro-1H-imidazo[4,5-c]pyridine-2,4-dione (100 mg, 215.35 μmol, 1 eq.) in MeOH (7 mL) and THF (3 mL) was added Pd/C (22.92 mg, 21.53 μmol, 10% purity, 0.1 eq.). The mixture was stirred at 55° C. for 4 h under H₂ (35 Psi). The mixture was filtered and concentrated under vacuum to get the residue. The residue was purified by prep-HPLC (column: Welch Xtimate C18 150*25 mm*5 μm; mobile phase: [H₂O (10 mM NH₄HCO₃)-ACN]; gradient: 30%-60% ACN over 11.0 min) to give 5-(4-(3-fluoro-5-(trifluoromethyl) phenoxy)pyridin-2-yl)-1-(2-hydroxyethyl)-3-methyl-3,5,6,7-tetrahydro-1H-imidazo[4,5-c]pyridine-2,4-dione (20.9 mg, 44.81 μmol, 20.81% yield) as a white solid. LCMS [M+H]⁺=467.1; ¹H NMR (400 MHz, DMSO-d₆) δ=8.37 (d, J=5.6 Hz, 1H), 7.66-7.60 (m, 1H), 7.53-7.49 (m, 1H), 7.48-7.43 (m, 2H), 6.92-6.80 (m, 1H), 4.97 (t, J=5.6 Hz, 1H), 4.25 (t, J=6.8 Hz, 2H), 3.72-3.63 (m, 2H), 3.57-3.52 (m, 2H), 3.35 (s, 3H), 2.91 (t, J=6.8 Hz, 2H).

### Example 52. Synthesis of 7-(4-(3-fluoro-5-(trifluoromethyl) phenoxy)pyridin-2-yl)-3-(3-hydroxypropyl)-1-methyl-6,7-dihydroimidazo[1,5-a]pyrazin-8(5H)-one (Compound 250)

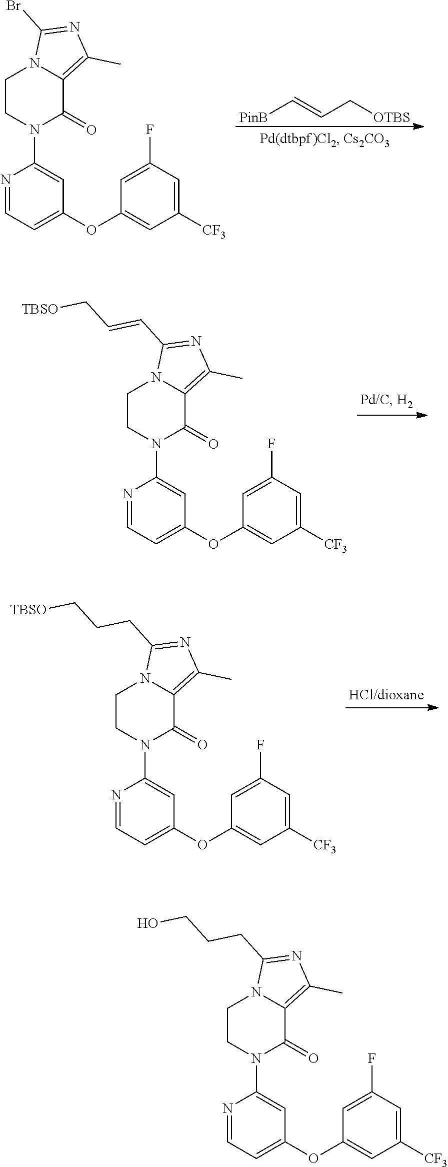

Step A: To a solution of 3-bromo-7-(4-(3-fluoro-5-(trifluoromethyl) phenoxy)pyridin-2-yl)-1-methyl-6,7-dihydroimidazo[1,5-a]pyrazin-8(5H)-one (150 mg, 309.13 μmol, 1 eq.) and (E)-tert-butyldimethyl ((3-(4,4,5,5-tetramethyl-1,3,2-dioxaborolan-2-yl) allyl)oxy) silane (110.66 mg, 370.96 μmol, 1.2 eq.) in H₂O (1 mL) and toluene (2 mL) was added Cs₂CO₃ (302.16 mg, 927.40 μmol, 3 eq.) and Pd (dtbpf) C12 ([1,1′-bis(di-tert-butylphosphino) ferrocene]dichloropalladium (II), CAS #95408-45-0, 20.15 mg, 30.91 μmol, 0.1 eq.), the mixture was stirred at 90° C. for 12 h under N₂ atmosphere. Then, the reaction mixture was diluted with EtOAc (50 mL), washed by water (30 mL) and brine (30 mL). The organic layer was dried over Na₂SO₄ and concentrated under vacuum. The residue was purified by flash silica gel chromatography (hexane to hexane/EtOAc=70/30, TLC: hexane/EtOAc=2/1, R_{f}=0.4) to yield (E)-3-(3-((tert-butyldimethylsilyl)oxy) prop-1-en-1-yl)-7-(4-(3-fluoro-5-(trifluoromethyl) phenoxy)pyridin-2-yl)-1-methyl-6,7-dihydroimidazo[1,5-a]pyrazin-8(5H)-one (140 mg, 218.50 μmol, 70.68% yield) as a yellow solid: ¹H NMR (400 MHz, DMSO-d₆) δ=8.40 (d, J=6.0 Hz, 1H), 7.72-7.43 (m, 4H), 6.91 (dd, J=2.0, 5.6 Hz, 1H), 6.80-6.68 (m, 1H), 6.54 (br s, 1H), 4.46-4.23 (m, 6H), 2.38 (s, 3H), 1.07 (s, 9H), 0.10 (s, 6H).

Step B: To a solution of (E)-3-(3-((tert-butyldimethylsilyl)oxy) prop-1-en-1-yl)-7-(4-(3-fluoro-5-(trifluoromethyl) phenoxy)pyridin-2-yl)-1-methyl-6,7-dihydroimidazo[1,5-a]pyrazin-8(5H)-one (120 mg, 208.10 μmol, 1 eq.) in MeOH (3 mL) was added Pd/C (60.00 mg, 56.38 μmol, 10% purity, 0.271 eq.), the mixture was stirred at 40° C. for 12 h under H₂ (30 Psi) atmosphere. Then, the reaction mixture was filtrated and concentrated under vacuum to yield 3-(3-((tert-butyldimethylsilyl)oxy)propyl)-7-(4-(3-fluoro-5-(trifluoromethyl) phenoxy)pyridin-2-yl)-1-methyl-6,7-dihydroimidazo[1,5-a]pyrazin-8 (5H)-one (130 mg, crude) as a white solid.

Step C: To a mixture of 3-(3-((tert-butyldimethylsilyl)oxy)propyl)-7-(4-(3-fluoro-5-(trifluoromethyl) phenoxy)pyridin-2-yl)-1-methyl-6,7-dihydroimidazo[1,5-a]pyrazin-8(5H)-one (130 mg, 224.65 μmol, 1 eq.) in HCl/dioxane (3 mL), the mixture was stirred at 25° C. for 2 h. The reaction mixture was concentrated under vacuum. The residue was purified by prep-HPLC (column: 40-WePure Biotech XP tC18 150×30 mm, 7 μm; mobile phase: [H₂O (0.05% NH₃H₂O+10 mM NH₄HCO₃)-ACN]; gradient: 33%-63% ACN over 10.0 min) to yield 7-(4-(3-fluoro-5-(trifluoromethyl) phenoxy)pyridin-2-yl)-3-(3-hydroxypropyl)-1-methyl-6,7-dihydroimidazo[1,5-a]pyrazin-8(5H)-one (43.37 mg, 91.85 μmol, 40.88% yield) as a white solid. LCMS [M+H]⁺=465.4; ¹H NMR (500 MHz, DMSO-d₆) δ=8.39 (d, J=5.6 Hz, 1H), 7.65 (br d, J=8.4 Hz, 1H), 7.61 (d, J=2.0 Hz, 1H), 7.54 (br d, J=9.6 Hz, 1H), 7.48 (s, 1H), 6.91 (dd, J=2.4, 6.0 Hz, 1H), 4.58 (t, J=5.2 Hz, 1H), 4.41-4.30 (m, 2H), 4.23-4.08 (m, 2H), 3.44 (q, J=6.0 Hz, 2H), 2.68 (t, J=7.6 Hz, 2H), 2.34 (s, 3H), 1.88-1.70 (m, 2H).

### Example 53. Synthesis of 5-(4-(3-fluoro-5-(trifluoromethyl) phenoxy)pyridin-2-yl)-1-(2-hydroxyethyl)-3,6-dimethyl-1,5-dihydro-4H-pyrazolo[3,4-d]pyrimidin-4-one (Compound 273)

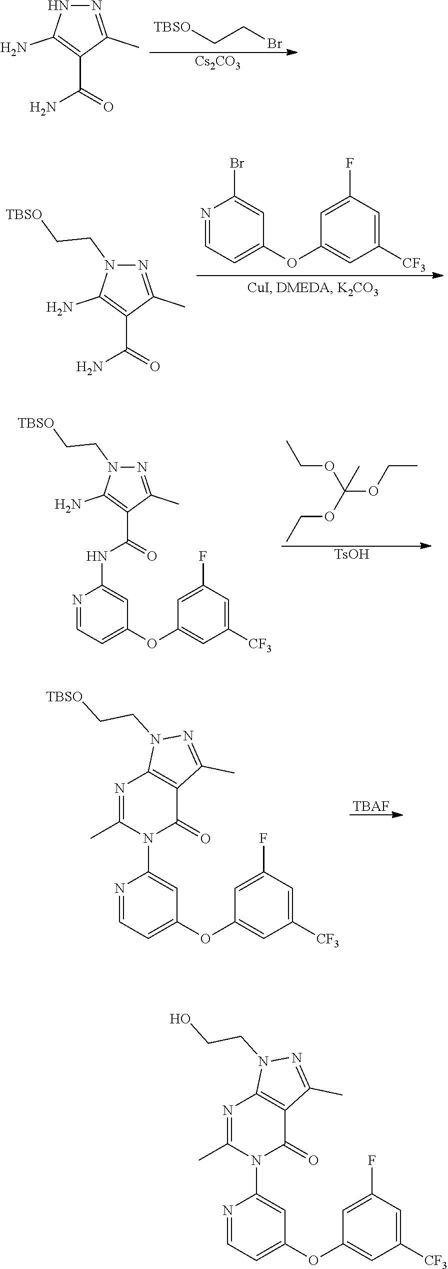

Step A: To a solution of 5-amino-3-methyl-1H-pyrazole-4-carboxamide (5.0 g, 14.87 mmol, 1 eq·q, 2·H₂SO₄ salt) and Cs₂CO₃ (24.22 g, 74.34 mmol, 5 eq.) in DMF (60 mL) was added (2-bromoethoxy) (tert-butyl)dimethylsilane (4.27 g, 17.84 mmol, 1.2 eq.) at 25° C. for 3 h. The reaction was slowly quenched with H₂O (200 mL). The layers were separated, then the aqueous layer was extracted with EtOAc (100 mL×3). The combined organic layers were washed with brine (100 mL×3), dried over Na₂SO₄, filtered and concentrated to yield a residue. The residue was purified by flash silica gel chromatography (hexanes to hexanes/EtOAc=10/90, TLC: hexanes/EtOAc=1/2) to give 5-amino-1-(2-((tert-butyldimethylsilyl)oxy)ethyl)-3-methyl-1H-pyrazole-4-carboxamide (2.4 g, 8.04 mmol, 54.09% yield) as a white solid: ¹H NMR (400 MHz, CDCl₃) δ=(br s, 2H), 5.40 (br s, 2H), 4.08-3.99 (m, 2H), 3.95-3.87 (m, 2H), 2.37 (s, 3H), 0.86 (s, 9H), 0.00 (s, 6H).

Step B: A solution of 5-amino-1-(2-((tert-butyldimethylsilyl)oxy)ethyl)-3-methyl-1H-pyrazole-4-carboxamide (960 mg, 3.22 mmol, 1 eq.), 2-bromo-4-(3-fluoro-5-(trifluoromethyl) phenoxy)pyridine (1.08 g, 3.22 mmol, 1 eq.), CuI (122.52 mg, 643.31 μmol, 0.2 eq.), DMEDA (113.42 mg, 1.29 mmol, 138.48 μL, 0.4 eq.) and K₂CO₃ (1.33 g, 9.65 mmol, 3 eq.) in dioxane (10 mL) was stirred at 110° C. for 12 h under N₂ atmosphere. The reaction was slowly quenched with H₂O (100 mL). The layers were separated, then the aqueous layer was extracted with EtOAc (50 mL×3). The combined organic layers were washed with brine (50 mL×3), dried over Na₂SO₄, filtered and concentrated to yield a residue. The residue was purified by flash silica gel chromatography (hexanes to EtOAc, TLC: hexanes/EtOAc=1/1) to give the crude product (1.6 g) as yellow oil. Then the crude product was purified by prep-HPLC (column: 58-Phenomenex Gemini NX C18 150×40 mm, 5 μm; mobile phase: [H₂O (10 mM NH₄HCO₃)-ACN]; gradient: 72%-92% ACN over 10.0 min) to give 5-amino-1-(2-((tert-butyldimethylsilyl)oxy)ethyl)-N-(4-(3-fluoro-5-(trifluoromethyl) phenoxy)pyridin-2-yl)-3-methyl-1H-pyrazole-4-carboxamide (1.4 g, 1.77 mmol, 27.52% yield) as colorless oil. LCMS [M+H]⁺=554.4; ¹H NMR (400 MHz, DMSO-d₆) δ=8.87 (s, 1H), 8.25 (d, J=5.6 Hz, 1H), 7.79 (d, J=2.0 Hz, 1H), 7.65 (d, J=8.4 Hz, 1H), 7.56 (d, J=9.6 Hz, 1H), 7.50 (s, 1H), 6.77 (dd, J=2.4, 5.6 Hz, 1H), 6.16 (s, 2H), 4.01-3.91 (m, 2H), 3.87-3.74 (m, 2H), 2.33 (s, 3H), 0.81 (s, 9H), −0.05 (s, 6H).

Step C: 5-amino-1-(2-((tert-butyldimethylsilyl)oxy)ethyl)-N-(4-(3-fluoro-5-(trifluoromethyl) phenoxy)pyridin-2-yl)-3-methyl-1H-pyrazole-4-carboxamide (490 mg, 885.08 μmol, 1 eq.) and TsOH (76.21 mg, 442.54 μmol, 0.5 eq.) was diluted in triethyl orthoacetate (5 mL). The solution was pumped {3 mL/min} to flow reactor 1 {HC, coil reactor, 3.175 (⅛″) mm, 30.000 mL, 200.0° C.}. The volume of cooling reactor was 10 mL (1/3.175 (⅛″)” PFA). The cooling temperature was 25° C. and the pressure was 3 MPa. The residence time of the flow reactor was 10.0 min. The mixture was collected with a bottle. The reaction was slowly quenched with H₂O (50 mL). The layers were separated then, the aqueous layer was extracted with EtOAc (30 mL×3). The combined organic layers were washed with brine (50 mL), dried over Na₂SO₄, filtered and concentrated to yield a residue. The residue was purified by flash silica gel chromatography (hexanes to hexanes/EtOAc=70/30, TLC: hexanes/EtOAc=3/1) to give 1-(2-((tert-butyldimethylsilyl)oxy)ethyl)-5-(4-(3-fluoro-5-(trifluoromethyl) phenoxy)pyridin-2-yl)-3,6-dimethyl-1,5-dihydro-4H-pyrazolo[3,4-d] pyrimidin-4-one (430 mg, 744.40 μmol, 84.11% yield) as a white solid: ¹H NMR (400 MHz, CDCl₃) δ=8.60 (d, J=5.6 Hz, 1H), 7.29 (s, 1H), 7.25 (s, 1H), 7.13-7.08 (m, 1H), 7.02-6.95 (m, 2H), 4.39 (t, J=5.6 Hz, 2H), 4.01 (t, J=5.6 Hz, 2H), 2.55 (s, 3H), 2.25 (s, 3H), 0.80 (s, 9H), −0.05 (s, 6H).

Step D: A solution of 1-(2-((tert-butyldimethylsilyl)oxy)ethyl)-5-(4-(3-fluoro-5-(trifluoromethyl) phenoxy)pyridin-2-yl)-3,6-dimethyl-1,5-dihydro-4H-pyrazolo[3,4-d] pyrimidin-4-one (200 mg, 346.23 μmol, 1 eq.) and TBAF (1 M, 1.73 mL, 5 eq.) in THF (5 mL) was stirred at 25° C. for 1 h. The reaction was slowly quenched with H₂O (50 mL). The layers were separated, then the aqueous layer was extracted with EtOAc (30 mL×3). The combined organic layers were washed with brine (50 mL), dried over Na₂SO₄, filtered and concentrated to yield a residue. The residue was purified by prep-HPLC (column: Welch Xtimate C18 150*25 mm*5 μm; mobile phase: [H₂O (10 mM NH₄HCO₃)-ACN]; gradient: 30%-60% ACN over 11.0 min) to give 5-(4-(3-fluoro-5-(trifluoromethyl) phenoxy)pyridin-2-yl)-1-(2-hydroxyethyl)-3,6-dimethyl-1,5-dihydro-4H-pyrazolo[3,4-d]pyrimidin-4-one (73.07 mg, 156.93 μmol, 45.33% yield) as a white solid. LCMS [M+H]⁺=464.2; ¹H NMR (400 MHz, DMSO-d₆) δ=8.59 (d, J=5.6 Hz, 1H), 7.72-7.60 (m, 2H), 7.57 (s, 1H), 7.39 (d, J=2.0 Hz, 1H), 7.26 (dd, J=2.4, 5.6 Hz, 1H), 4.89 (t, J=5.2 Hz, 1H), 4.24 (t, J=5.6 Hz, 2H), 3.79 (q, J=5.2 Hz, 2H), 2.39 (s, 3H), 2.14 (s, 3H).

### Example 54. Synthesis of 5-(4-((7-fluoro-1-methyl-1H-indazol-5-yl)oxy)pyridin-2-yl)-1-(2-hydroxyethyl)-3-methyl-1,5,6,7-tetrahydro-4H-pyrazolo[4,3-c]pyridin-4-one (Compound 274) and 5-(4-((7-fluoro-2-methyl-2H-indazol-5-yl)oxy)pyridin-2-yl)-1-(2-hydroxyethyl)-3-methyl-1,5,6,7-tetrahydro-4H-pyrazolo[4,3-c]pyridin-4-one (Compound 275)

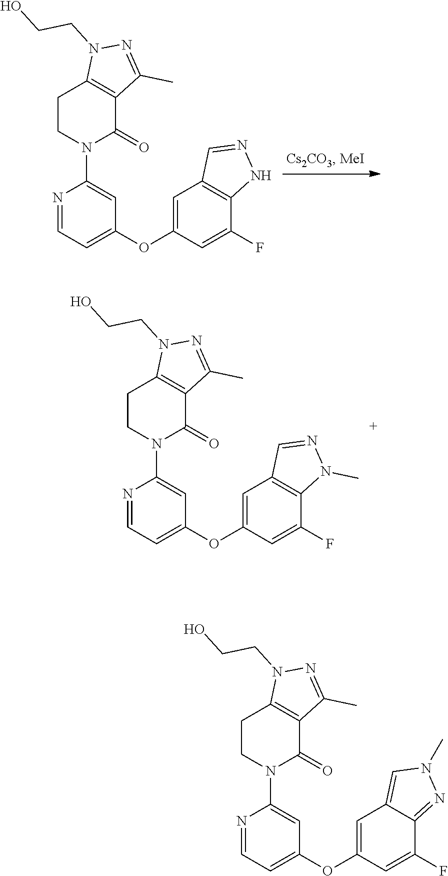 [SMILES: Cc1nn(CCO)c2c1C(=O)N(c1cc(Oc3cc(F)c4[nH]ncc4c3)ccn1)CC2 ; Cc1nn(CCO)c2c1C(=O)N(c1cc(Oc3cc(F)c4c(cnn4C)c3)ccn1)CC2 ; Cc1nn(CCO)c2c1C(=O)N(c1cc(Oc3cc(F)c4nn(C)cc4c3)ccn1)CC2]

Step A: To a solution of 5-(4-((7-fluoro-1H-indazol-5-yl)oxy)pyridin-2-yl)-1-(2-hydroxyethyl)-3-methyl-1,5,6,7-tetrahydro-4H-pyrazolo[4,3-c]pyridin-4-one (60 mg, 142.04 μmol, 1 eq.) in DMF (2 mL) was added Cs₂CO₃ (138.84 mg, 426.12 μmol, 3 eq.) and MeI (40.32 mg, 284.08 μmol, 17.69 μL, 2 eq.), the mixture was stirred at 60° C. for 2 h under N₂ atmosphere. The reaction mixture was quenched by addition NH₄Cl (5 mL), then extracted with EtOAc (5 mL×3). The combined organic layers were dried over Na₂SO₄, filtered and concentrated under reduced pressure. The residue was purified by prep-HPLC (column: 40-WePure Biotech XP tC18 150×30 mm, 7 μm; mobile phase: [H₂O (0.05% NH₃H₂O+10 mM NH₄HCO₃)-ACN]; gradient: 28%-58% ACN over 10.0 min) to yield 5-(4-((7-fluoro-1-methyl-1H-indazol-5-yl)oxy)pyridin-2-yl)-1-(2-hydroxyethyl)-3-methyl-1,5,6,7-tetrahydro-4H-pyrazolo[4,3-c]pyridin-4-one (32.37 mg, 73.65 μmol, 25.93% yield) and 5-(4-((7-fluoro-2-methyl-2H-indazol-5-yl)oxy)pyridin-2-yl)-1-(2-hydroxyethyl)-3-methyl-1,5,6,7-tetrahydro-4H-pyrazolo[4,3-c]pyridin-4-one (20.85 mg, 47.77 μmol, 33.63% yield) as white solid.

Compound 274: LCMS [M+H]⁺=437.2; ¹H NMR (500 MHz, DMSO-d₆) δ=8.30 (d, J=5.5 Hz, 1H), 8.13 (d, J=2.0 Hz, 1H), 7.44 (d, J=1.5 Hz, 1H), 7.31 (d, J=2.0 Hz, 1H), 7.23 (dd, J=2.0, 12.0 Hz, 1H), 6.76 (dd, J=2.0, 6.0 Hz, 1H), 4.89 (br s, 1H), 4.24 (t, J=6.0 Hz, 2H), 4.19 (s, 3H), 4.03 (t, J=5.5 Hz, 2H), 3.68 (br t, J=5.0 Hz, 2H), 3.02 (t, J=6.5 Hz, 2H), 2.28 (s, 3H).

Compound 275: LCMS [M+H]⁺=437.1; ¹H NMR (500 MHz, DMSO-d₆) δ=8.48 (d, J=2.5 Hz, 1H), 8.29 (d, J=5.5 Hz, 1H), 7.36 (d, J=2.0 Hz, 1H), 7.32 (d, J=2.0 Hz, 1H), 7.01 (dd, J=2.0, 12.0 Hz, 1H), 6.76 (dd, J=2.0, 5.5 Hz, 1H), 4.89 (br s, 1H), 4.24 (t, J=6.5 Hz, 2H), 4.21 (s, 3H), 4.03 (t, J=5.0 Hz, 2H), 3.68 (br t, J=5.0 Hz, 2H), 3.02 (t, J=6.5 Hz, 2H), 2.28 (s, 3H).

### Example 55. Synthesis of rac-7-(4-(3-fluoro-5-(trifluoromethyl) phenoxy)pyridin-2-yl)-3-((1R,2S)-2-(hydroxymethyl)cyclopropyl)-1-methyl-6,7-dihydroimidazo[1,5-a]pyrazin-8(5H)-one (Compound 276)

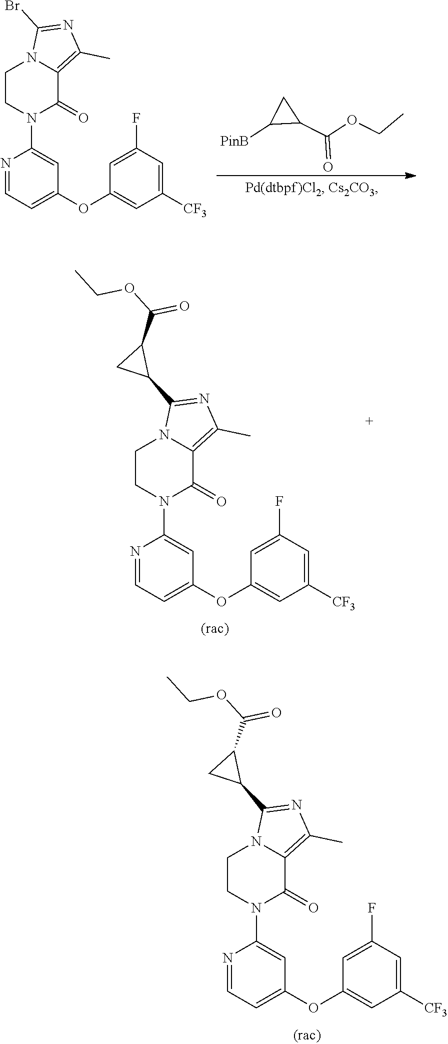

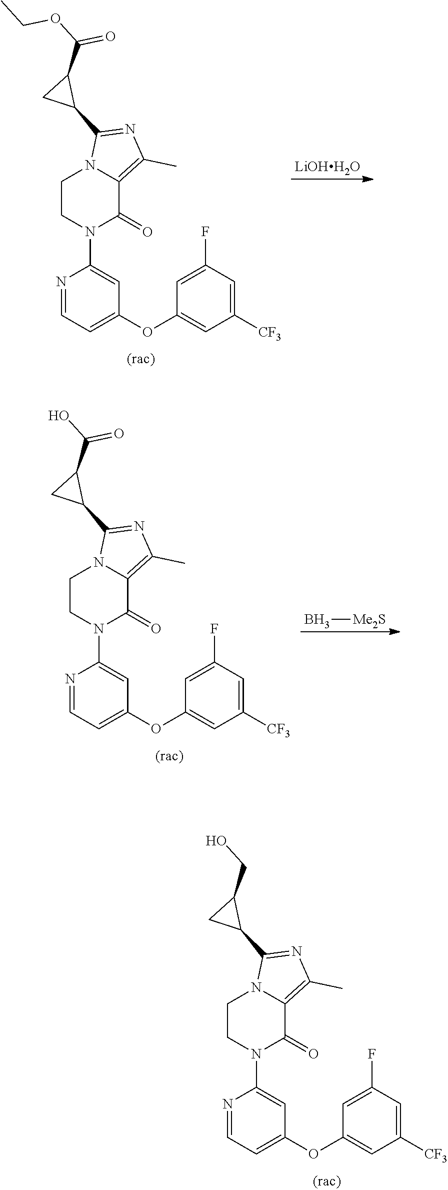

Step A: To a solution of 3-bromo-7-(4-(3-fluoro-5-(trifluoromethyl) phenoxy)pyridin-2-yl)-1-methyl-6,7-dihydroimidazo[1,5-a]pyrazin-8(5H)-one (760 mg, 1.57 mmol, 1 eq.), ethyl 2-(4,4,5,5-tetramethyl-1,3,2-dioxaborolan-2-yl)cyclopropane-1-carboxylate (676.92 mg, 2.82 mmol, 1.8 eq.) and Cs₂CO₃ (1.53 g, 4.70 mmol, 3 eq.) in toluene (15 mL) and H₂O (5 mL) was added Pd (dtbpf) C12 (102.08 mg, 156.63 μmol, 0.1 eq.) and the mixture was stirred at 90° C. for 12 h under N₂. Then, the reaction mixture was cooled, water (60 mL) was added then the organics were extracted with EtOAc (60 mL×3). The combined organic layers were washed with brine (60 mL×2), dried over Na₂SO₄, and filtered. The filtrate was concentrated under reduced pressure to give the crude product. The crude product was purified twice by flash silica gel chromatography (hexane to hexane/EtOAc=50/50), then (DCM to DCM/MeOH=95/5) to give rac-ethyl (1R,2S)-2-(7-(4-(3-fluoro-5-(trifluoromethyl) phenoxy)pyridin-2-yl)-1-methyl-8-oxo-5,6,7,8-tetrahydroimidazo[1,5-a]pyrazin-3-yl)cyclopropane-1-carboxylate (240 mg, 439.76 μmol, 28.08% yield) as a yellow solid and rac-ethyl (1R,2R)-2-(7-(4-(3-fluoro-5-(trifluoromethyl) phenoxy)pyridin-2-yl)-1-methyl-8-oxo-5,6,7,8-tetrahydroimidazo[1,5-a]pyrazin-3-yl)cyclopropane-1-carboxylate (170 mg, 278.71 μmol, 17.79% yield) as a yellow solid: rac-ethyl (1R,2S)-2-(7-(4-(3-fluoro-5-(trifluoromethyl) phenoxy)pyridin-2-yl)-1-methyl-8-oxo-5,6,7,8-tetrahydroimidazo[1,5-a]pyrazin-3-yl)cyclopropane-1-carboxylate: LCMS [M+H]⁺=519.0; ¹H NMR (400 MHz, CDCl₃) δ=8.33 (d, J=5.6 Hz, 1H), 7.77 (d, J=2.4 Hz, 1H), 7.24-7.14 (m, 2H), 7.08-6.97 (m, 1H), 6.71 (dd, J=2.4, 5.6 Hz, 1H), 4.62-4.52 (m, 1H), 4.39-4.30 (m, 1H), 4.28-4.21 (m, 1H), 4.20-4.12 (m, 1H), 4.07-3.94 (m, 2H), 2.55 (s, 3H), 2.43-2.32 (m, 1H), 2.26-2.13 (m, 1H), 2.08-1.93 (m, 1H), 1.57-1.53 (m, 1H), 1.12 (t, J=7.2 Hz, 3H).

rac-ethyl (1R,2R)-2-(7-(4-(3-fluoro-5-(trifluoromethyl) phenoxy)pyridin-2-yl)-1-methyl-8-oxo-5,6,7,8-tetrahydroimidazo[1,5-a]pyrazin-3-yl)cyclopropane-1-carboxylate: LCMS [M+H]⁺=519.0; ¹H NMR (400 MHz, CDCl₃) δ=8.34 (d, J=6.0 Hz, 1H), 7.78 (d, J=2.0 Hz, 1H), 7.21-7.18 (m, 2H), 7.03 (d, J=9.2 Hz, 1H), 6.72 (dd, J=2.0, 5.6 Hz, 1H), 4.55-4.50 (m, 2H), 4.27 (t, J=5.6 Hz, 2H), 4.20 (d, J=6.8 Hz, 2H), 2.54-2.49 (m, 3H), 2.45-2.32 (m, 2H), 1.78-1.67 (m, 2H), 1.28-1.24 (m, 3H).

Step B: To a solution of rac-ethyl (1R,2S)-2-(7-(4-(3-fluoro-5-(trifluoromethyl) phenoxy)pyridin-2-yl)-1-methyl-8-oxo-5,6,7,8-tetrahydroimidazo[1,5-a]pyrazin-3-yl)cyclopropane-1-carboxylate (100 mg, 192.88 μmol, 1 eq.) in H₂O (1 mL) and THF (3 mL) was added LiOH·H₂O (48.56 mg, 1.16 mmol, 6 eq.) and the mixture was stirred at 25° C. for 3 h. Then water (20 mL) was added to the reaction mixture and the organics were extracted with EtOAc (30 mL). The aqueous layer was acidified with 1 N HCl to adjust the pH to 3-4 and extracted with (DCM: i-PrOH=3:1) (20 mL×2). These combined organic layers were dried over Na₂SO₄, and filtered. The filtrate was concentrated under reduced pressure to give rac-(1R,2S)-2-(7-(4-(3-fluoro-5-(trifluoromethyl) phenoxy)pyridin-2-yl)-1-methyl-8-oxo-5,6,7,8-tetrahydroimidazo[1,5-a]pyrazin-3-yl)cyclopropane-1-carboxylic acid (80 mg, crude) as brown oil, which was used for the next step directly without further purification. LCMS [M+H]⁺=491.0.

Step C: To a solution of rac-(1R,2S)-2-(7-(4-(3-fluoro-5-(trifluoromethyl) phenoxy)pyridin-2-yl)-1-methyl-8-oxo-5,6,7,8-tetrahydroimidazo[1,5-a]pyrazin-3-yl)cyclopropane-1-carboxylic acid (60 mg, 122.35 μmol, 1 eq.) in THF (1.5 mL) was added BH₃—Me₂S (10 M, 122.35 μL, 10 eq.) at 0° C. The mixture was stirred at 0° C. for 1 h under N₂. Then, MeOH (4 mL) was added into the mixture dropwise at 0° C. The mixture was stirred at 20° C. for 0.5 h. The mixture was concentrated under reduced pressure to give a residue. The crude product was purified by Prep-HPLC (column: Welch Xtimate C18 150*25 mm*5 μm; mobile phase: [H₂O (10 mM NH₄HCO₃)-ACN]; gradient: 46%-76% ACN over 11.0 min) to give rac-7-(4-(3-fluoro-5-(trifluoromethyl) phenoxy)pyridin-2-yl)-3-((1R,2S)-2-(hydroxymethyl)cyclopropyl)-1-methyl-6,7-dihydroimidazo[1,5-a]pyrazin-8(5H)-one (15.52 mg, 32.58 μmol, 26.63% yield) as a white solid. LCMS [M+H]⁺=477.1; ¹H NMR (400 MHz, DMSO-d₆) δ=8.40 (d, J=5.6 Hz, 1H), 7.65 (d, J=8.4 Hz, 1H), 7.59 (d, J=2.4 Hz, 1H), 7.55 (td, J=2.0, 9.6 Hz, 1H), 7.49 (s, 1H), 6.91 (dd, J=2.4, 5.6 Hz, 1H), 4.58 (t, J=5.2 Hz, 1H), 4.40-4.21 (m, 4H), 3.56-3.50 (m, 1H), 2.98-2.89 (m, 1H), 2.33 (s, 3H), 2.15-2.07 (m, 1H), 1.53-1.43 (m, 1H), 1.13-0.98 (m, 2H).

### Example 56. Synthesis of rac-7-(4-(3-fluoro-5-(trifluoromethyl) phenoxy)pyridin-2-yl)-3-((1R,2R)-2-(hydroxymethyl)cyclopropyl)-1-methyl-6,7-dihydroimidazo[1,5-a]pyrazin-8(5H)-one (Compound 277)

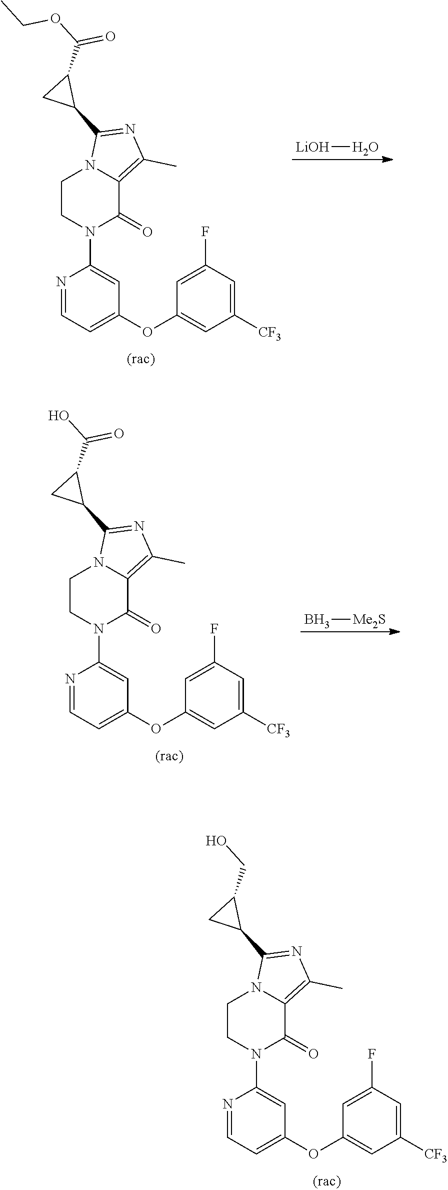

Step A: To a solution of rac-ethyl (1R,2R)-2-(7-(4-(3-fluoro-5-(trifluoromethyl) phenoxy)pyridin-2-yl)-1-methyl-8-oxo-5,6,7,8-tetrahydroimidazo[1,5-a]pyrazin-3-yl)cyclopropane-1-carboxylate (160 mg, 308.61 μmol, 1 eq.) in THF (2 mL) was added a solution of LiOH. H₂O (64.75 mg, 1.54 mmol, 5 eq.) in H₂O (0.8 mL) slowly under N₂ atmosphere at 0° C. The resulting mixture was stirred at 30° C. for 2 h under N₂ atmosphere. The reaction mixture was concentrated to remove most of solvent. The residue was diluted with H₂O (20 mL). Then, 2M HCl was added drop-wise at 0° C. until pH to 3-4. The mixture was then extracted with DCM/i-PrOH (20 mL×3, v/v=3:1). The separated organic layers were combined, dried over Na₂SO₄ and filtered. The filtrate was concentrated under reduced pressure to give rac-(1R,2R)-2-(7-(4-(3-fluoro-5-(trifluoromethyl) phenoxy)pyridin-2-yl)-1-methyl-8-oxo-5,6,7,8-tetrahydroimidazo[1,5-a]pyrazin-3-yl)cyclopropane-1-carboxylic acid (160 mg, crude) as a yellow solid, which was used for the next step directly without further purification. LCMS [M+H]⁺=491.5; ¹H NMR (400 MHz, DMSO-d₆) δ=8.45 (d, J=5.6 Hz, 1H), 7.69-7.65 (m, 1H), 7.61-7.55 (m, 2H), 7.50 (s, 1H), 7.00 (dd, J=2.4, 5.6 Hz, 1H), 4.57-4.37 (m, 4H), 2.74-2.66 (m, 1H), 2.48 (s, 3H), 2.40-2.34 (m, 1H), 1.79-1.68 (m, 1H), 1.67-1.56 (m, 1H).

Step B: To a solution of rac-(1R,2R)-2-(7-(4-(3-fluoro-5-(trifluoromethyl) phenoxy)pyridin-2-yl)-1-methyl-8-oxo-5,6,7,8-tetrahydroimidazo[1,5-a]pyrazin-3-yl)cyclopropane-1-carboxylic acid (140 mg, 242.66 μmol, 1 eq.) in THF (5 mL) was added BH₃—Me₂S (10 M, 194.12 μL, 8 eq.) slowly under N₂ atmosphere at 0° C. The resulting mixture was stirred at 0° C. for 2 h. Then, MeOH (5 mL) was added slowly into the mixture to quench the reaction and stirred for 10 min at 0° C. The reaction mixture was concentrated under vacuum to give a residue, which was purified by prep-HPLC [Column: Welch Xtimate C18 150*25 mm*5 μm, Condition: 38-68% (A: H₂O (10 mM NH₄CO₃), B: CH₃CN), flow rate: 25 mL/min] to give rac-7-(4-(3-fluoro-5-(trifluoromethyl) phenoxy)pyridin-2-yl)-3-((1R,2R)-2-(hydroxymethyl)cyclopropyl)-1-methyl-6,7-dihydroimidazo[1,5-a]pyrazin-8(5H)-one (11.20 mg, 23.51 μmol, 9.69% yield) as a white solid. LCMS [M+H]⁺=477.2; ¹H NMR (400 MHz, DMSO-d₆) δ=8.39 (d, J=5.6 Hz, 1H), 7.66-7.59 (m, 2H), 7.57-7.50 (m, 1H), 7.48 (s, 1H), 6.91 (dd, J=2.0, 5.6 Hz, 1H), 4.68 (t, J=5.6 Hz, 1H), 4.44-4.25 (m, 4H), 3.62-3.45 (m, 1H), 3.32-3.30 (m, 1H), 2.31 (s, 3H), 1.92-1.84 (m, 1H), 1.57-1.46 (m, 1H), 1.12-1.01 (m, 1H), 0.99-0.88 (m, 1H).

### Example 57. Synthesis of 1-cyclopropyl-7-(4-(3-fluoro-5-(trifluoromethyl) phenoxy)pyridin-2-yl)-3-(2-hydroxyethyl)-6,7-dihydroimidazo[1,5-a]pyrazin-8(5H)-one (Compound 284)

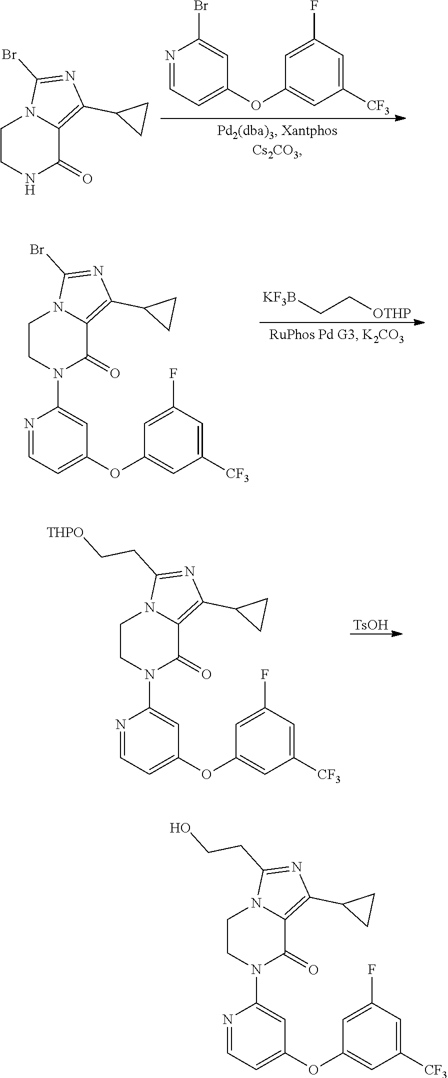

Step A: A mixture of 3-bromo-1-cyclopropyl-6,7-dihydroimidazo[1,5-a]pyrazin-8(5H)-one (400 mg, 1.56 mmol, 1 eq.), 2-bromo-4-(3-fluoro-5-(trifluoromethyl) phenoxy)pyridine (524.92 mg, 1.56 mmol, 1 eq.), Pd₂(dba)₃ (143.03 mg, 156.19 μmol, 0.1 eq.), Xantphos (90.37 mg, 156.19 μmol, 0.1 eq.) and Cs₂CO₃ (1.02 g, 3.12 mmol, 2 eq.) in dioxane (8 mL) was degassed and purged with N₂ three times, and then the mixture was stirred at 90° C. for 14 h under a N₂ atmosphere. The reaction mixture was filtered and concentrated under reduced pressure to give a residue. The residue was purified by flash silica gel chromatography (hexanes to hexanes/EtOAc=3/2, TLC: hexanes/EtOAc=2/1, R_{f}=0.4) to yield 3-bromo-1-cyclopropyl-7-(4-(3-fluoro-5-(trifluoromethyl) phenoxy)pyridin-2-yl)-6,7-dihydroimidazo[1,5-a]pyrazin-8(5H)-one (500 mg, 977.96 μmol, 62.61% yield) as a yellow solid: ¹H NMR (400 MHz, DMSO-d₆) δ=8.41 (d, J=5.6 Hz, 1H), 7.65 (d, J=8.4 Hz, 1H), 7.61 (d, J=2.4 Hz, 1H), 7.58-7.53 (m, 1H), 7.50 (s, 1H), 6.97-6.91 (m, 1H), 4.46-4.37 (m, 2H), 4.23-4.12 (m, 2H), 2.64-2.57 (m, 1H), 0.95-0.88 (m, 2H), 0.86-0.80 (m, 2H).

Step B: A mixture of 3-bromo-1-cyclopropyl-7-(4-(3-fluoro-5-(trifluoromethyl) phenoxy)pyridin-2-yl)-6,7-dihydroimidazo[1,5-a]pyrazin-8(5H)-one (300 mg, 586.78 μmol, 1 eq.), trifluoro (2-((tetrahydro-2H-pyran-2-yl)oxy)ethyl)-24-borane, potassium salt (415.58 mg, 1.76 mmol, 3 eq.), RuPhos Pd G3 (49.08 mg, 58.68 μmol, 0.1 eq.), K₂CO₃ (243.30 mg, 1.76 mmol, 3 eq.) and H₂O (1.2 mL) in toluene (6 mL) was degassed and purged with N₂ three times, and then the mixture was stirred at 100° C. for 14 h under a N₂ atmosphere. The reaction mixture was filtered and concentrated under reduced pressure to give a residue. The residue was purified by flash silica gel chromatography (hexanes to hexanes/EtOAc=1/1, TLC: hexanes/EtOAc=0/1, R_{f}=0.5) to yield 1-cyclopropyl-7-(4-(3-fluoro-5-(trifluoromethyl) phenoxy)pyridin-2-yl)-3-(2-((phosphaneyl-t)oxy)ethyl)-6,7-dihydroimidazo[1,5-a]pyrazin-8(5H)-one (300 mg, 409.96 μmol, 69.87% yield) as yellow oil. LCMS [M+H]⁺=561.3.

Step C: To a solution of 1-cyclopropyl-7-(4-(3-fluoro-5-(trifluoromethyl) phenoxy)pyridin-2-yl)-3-(2-((phosphaneyl-t)oxy)ethyl)-6,7-dihydroimidazo[1,5-a]pyrazin-8(5H)-one (350 mg, 624.40 μmol, 7.80 μL, 1 eq.) in THF (7 mL) was added TsOH (215.04 mg, 1.25 mmol, 2 eq.) and H₂O (1.35 mL). The mixture was stirred at 60° C. for 14 h. The reaction mixture was quenched by the addition sat. NaHCO₃ (20 mL), then diluted with H₂O (10 mL) and extracted with DCM (20 mL×3). The combined organic layers were dried over Na₂SO₄, filtered and concentrated under reduced pressure to give a residue. The residue was purified by flash silica gel chromatography (DCM to DCM/MeOH=96/4, TLC: DCM/MeOH=10/1, R_{f}=0.5) to give a white solid (200 mg, 65.89% yield). Then, EtOH (2 mL) was added to the solid and stirred at 80° C. until all solid was dissolved and a clear solution was obtained. The mixture was cooled down to 20° C. The resulting crystals were filtered, and dried under reduced pressure to yield 1-cyclopropyl-7-(4-(3-fluoro-5-(trifluoromethyl) phenoxy)pyridin-2-yl)-3-(2-hydroxyethyl)-6,7-dihydroimidazo[1,5-a]pyrazin-8(5H)-one (99.03 mg) as a white solid. LCMS [M+H]⁺=477.2; ¹H NMR (400 MHz, DMSO-d₆) δ=8.40 (d, J=5.6 Hz, 1H), 7.67-7.61 (m, 2H), 7.57-7.51 (m, 1H), 7.49 (s, 1H), 6.93-6.89 (m, 1H), 4.78 (t, J=5.6 Hz, 1H), 4.38-4.30 (m, 2H), 4.25-4.16 (m, 2H), 3.65 (q, J=6.4 Hz, 2H), 2.77 (t, J=6.4 Hz, 2H), 2.64-2.56 (m, 1H), 0.89-0.80 (m, 4H).

### Example 58. Synthesis of 1-cyclopropyl-7-(4-(3-fluoro-5-(trifluoromethyl) phenoxy)pyridin-2-yl)-3-(hydroxymethyl)-6,7-dihydroimidazo[1,5-a]pyrazin-8(5H)-one (Compound 292) and 1-cyclopropyl-7-(4-(3-fluoro-5-(trifluoromethyl) phenoxy)pyridin-2-yl)-6,7-dihydroimidazo[1,5-a]pyrazin-8(5H)-one (Compound 293)

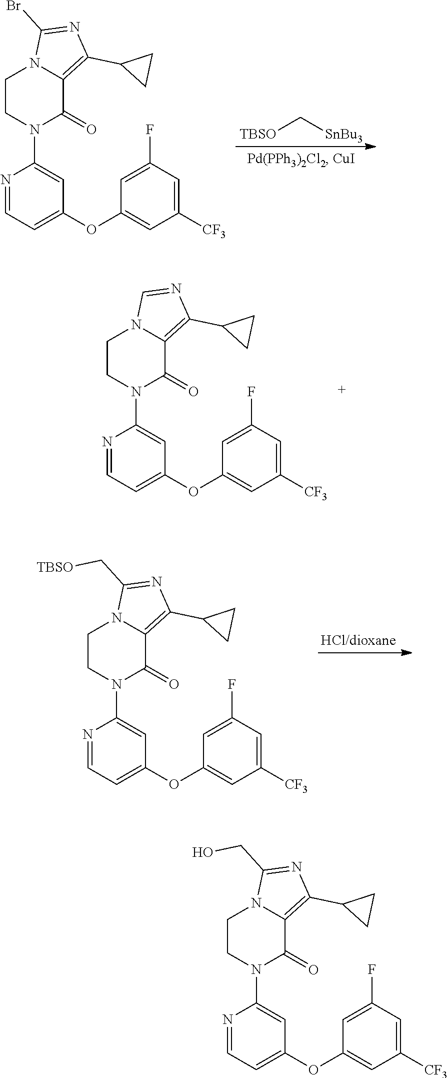

Step A: A mixture of 3-bromo-1-cyclopropyl-7-(4-(3-fluoro-5-(trifluoromethyl) phenoxy)pyridin-2-yl)-6,7-dihydroimidazo[1,5-a]pyrazin-8(5H)-one (200 mg, 391.19 μmol, 1 eq.), tert-butyldimethyl ((tributylstannyl) methoxy) silane (510.90 mg, 1.17 mmol, 3 eq.), CuI (22.35 mg, 117.36 μmol, 25.84 μL, 0.3 eq.), Pd(PPh₃)₂Cl₂ (27.46 mg, 39.12 μmol, 0.1 eq.) in dioxane (8 mL) was degassed and purged with N₂ three times, then the mixture was stirred at 100° C. for 14 h under a N₂ atmosphere. The reaction mixture was filtered and concentrated under reduced pressure to give a residue. The residue was purified by flash silica gel chromatography (hexanes to hexanes/EtOAc=1/1) to yield 3-(((tert-butyldimethylsilyl)oxy)methyl)-1-cyclopropyl-7-(4-(3-fluoro-5-(trifluoromethyl) phenoxy)pyridin-2-yl)-6,7-dihydroimidazo[1,5-a]pyrazin-8(5H)-one (80 mg, 83.24 μmol, 21.28% yield) as yellow oil, and 1-cyclopropyl-7-(4-(3-fluoro-5-(trifluoromethyl) phenoxy)pyridin-2-yl)-6,7-dihydroimidazo[1,5-a]pyrazin-8(5H)-one (crude). LCMS [M+H]⁺=577.3.

The crude 1-cyclopropyl-7-(4-(3-fluoro-5-(trifluoromethyl) phenoxy)pyridin-2-yl)-6,7-dihydroimidazo[1,5-a]pyrazin-8(5H)-one was purified by prep-HPLC (column: Welch Xtimate C 18 150*25 mm*5 μm; mobile phase: [H₂O (10 mM NH₄HCO₃)-ACN]; gradient: 40%-70% ACN over 11.0 min) to yield pure 1-cyclopropyl-7-(4-(3-fluoro-5-(trifluoromethyl) phenoxy)pyridin-2-yl)-6,7-dihydroimidazo[1,5-a]pyrazin-8(5H)-one (20.01 mg) as a white solid. LCMS [M+H]⁺=433.1; ¹H NMR (400 MHz, DMSO-d₆) δ=8.40 (d, J=5.6 Hz, 1H), 7.72 (s, 1H), 7.65 (d, J=8.4 Hz, 1H), 7.62 (d, J=2.4 Hz, 1H), 7.58-7.53 (m, 1H), 7.50 (s, 1H), 6.92 (dd, J=2.4, 5.6 Hz, 1H), 4.39-4.33 (m, 2H), 4.32-4.25 (m, 2H), 2.63-2.55 (m, 1H), 0.93-0.80 (m, 4H).

Step B: To a solution of 3-(((tert-butyldimethylsilyl)oxy)methyl)-1-cyclopropyl-7-(4-(3-fluoro-5-(trifluoromethyl) phenoxy)pyridin-2-yl)-6,7-dihydroimidazo[1,5-a]pyrazin-8(5H)-one (300 mg, 520.24 μmol, 1 eq.) in DCM (3 mL) was added HCl/dioxane (3 mL) and the mixture was stirred at 25° C. for 0.5 h. The reaction mixture was quenched by addition sat. NaHCO₃ (10 mL), and extracted with DCM (10 mL×3). The combined organic layers were dried over Na₂SO₄, filtered and concentrated under reduced pressure to give a residue. The residue was purified by flash silica gel chromatography (DCM to DCM/MeOH=96/4, TLC: DCM/MeOH=10/1, R_{f}=0.4) to give a white solid (160 mg, 65.44% yield). Then, EtOH (16 mL) was added and the suspension was stirred at 80° C. until all solid was dissolved and a clear solution was obtained. The mixture was cooled down to 20° C. The resulting crystals were filtered, and dried under reduced pressure to yield 1-cyclopropyl-7-(4-(3-fluoro-5-(trifluoromethyl) phenoxy)pyridin-2-yl)-3-(hydroxymethyl)-6,7-dihydroimidazo[1,5-a]pyrazin-8(5H)-one (114.05 mg, 255.50 μmol, 73.84% yield) as a white solid. LCMS [M+H]⁺=463.1; ¹H NMR (400 MHz, DMSO-d₆) δ=8.40 (d, J=5.6 Hz, 1H), 7.67-7.61 (m, 2H), 7.58-7.52 (m, 1H), 7.49 (s, 1H), 6.95-6.90 (m, 1H), 5.47 (t, J=5.6 Hz, 1H), 4.48 (d, J=6.0 Hz, 2H), 4.39-4.32 (m, 2H), 4.32-4.24 (m, 2H), 2.64-2.56 (m, 1H), 0.91-0.77 (m, 4H).

### Example 59. Synthesis of 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxamide (Compound 302)

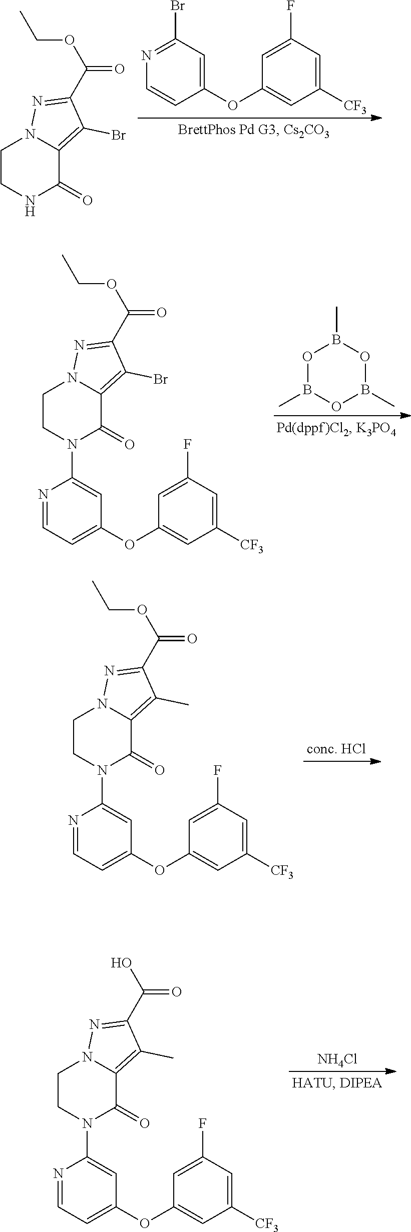

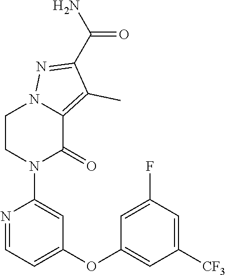 [SMILES: Cc1c(C(N)=O)nn2c1C(=O)N(c1cc(Oc3cc(F)cc(C(F)(F)F)c3)ccn1)CC2]

Step A: To a solution of ethyl 3-bromo-4-oxo-4,5,6,7-tetrahydropyrazolo[1,5-a]pyrazine-2-carboxylate (140 mg, 485.95 μmol, 1 eq.) and 2-bromo-4-(3-fluoro-5-(trifluoromethyl) phenoxy)pyridine (179.65 mg, 534.54 μmol, 1.1 eq.) in dioxane (3 mL) was added BrettPhos Pd G3 (44.05 mg, 48.59 μmol, 0.1 eq.) and Cs₂CO₃ (474.99 mg, 1.46 mmol, 3 eq.), the mixture was stirred at 100° C. for 2 h under a N₂ atmosphere and microwave irradiation. The reaction mixture was concentrated under vacuum. The residue was purified by flash silica gel chromatography (DCM to DCM/MeOH=95/5, TLC: DCM/MeOH=10/1, R_{f}=0.4) to yield ethyl 3-bromo-5-(4-(3-fluoro-5-(trifluoromethyl) phenoxy)pyridin-2-yl)-4-oxo-4,5,6,7-tetrahydropyrazolo[1,5-a]pyrazine-2-carboxylate (100 mg, 128.85 μmol, 26.52% yield) as a white solid. LCMS [M+H]⁺=544.8.

Step B: To a solution of ethyl 3-bromo-5-(4-(3-fluoro-5-(trifluoromethyl) phenoxy)pyridin-2-yl)-4-oxo-4,5,6,7-tetrahydropyrazolo[1,5-a]pyrazine-2-carboxylate (70 mg, 128.85 μmol, 1 eq.) and trimethylboroxine (80.88 mg, 644.25 μmol, 90.06 μL, 5 eq.) in dioxane (2 mL) and H₂O (1 mL) was added Pd(dppf) C12 (9.43 mg, 12.89 μmol, 0.1 eq.) and K₂CO₃ (53.42 mg, 386.55 μmol, 3 eq.), the mixture was stirred at 80° C. for 2 h under a N₂ atmosphere. The reaction mixture was diluted with EtOAc (20 mL), washed by water (10 mL) and brine (10 mL). The organic layer was dried over Na₂SO₄ and concentrated under vacuum. The residue was purified by flash silica gel chromatography (DCM to DCM/MeOH=95/5, TLC: DCM/MeOH=10/1, R_{f}=0.6) to yield ethyl 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxylate (50 mg, 62.71 μmol, 48.67% yield) as a yellow solid. LCMS [M+H]⁺=479.2.

Step C: A mixture of ethyl 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxylate (90 mg, 131.69 μmol, 1 eq.) in HCl (8 M, 3 mL, 182.25 eq.) was stirred at 80° C. for 1 h. The reaction mixture was concentrated under vacuum to yield 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxylic acid (85 mg, crude) as a white solid. LCMS [M+H]⁺=451.2.

Step D: To a solution of 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxylic acid (80 mg, 177.64 μmol, 1 eq.) and NH₄Cl (28.51 mg, 532.93 μmol, 3 eq.) in DMF (2 mL) was added HATU (101.32 mg, 266.46 μmol, 1.5 eq.) and DIPEA (114.79 mg, 888.21 μmol, 154.71 μL, 5 eq.), the mixture was stirred at 25° C. for 2 h under a N₂ atmosphere. The reaction mixture was filtrated and concentrated under vacuum. The residue was purified by prep-HPLC (column: 40-WePure Biotech XP tC18 150×30 mm, 7 μm; mobile phase: [H₂O (0.05% NH₃H₂O+10 mM NH₄HCO₃)-ACN]; gradient: 36%-66% B over 10.0 min) to yield 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxamide (18.18 mg, 39.42 μmol, 22.19% yield) as a white solid. LCMS [M+H]⁺=450.0; ¹H NMR (400 MHz, DMSO-d₆) δ=8.45 (d, J=5.6 Hz, 1H), 7.67 (br d, J=8.4 Hz, 1H), 7.62 (d, J=2.4 Hz, 1H), 7.60-7.49 (m, 3H), 7.28 (br s, 1H), 6.98 (dd, J=2.0, 5.6 Hz, 1H), 4.49 (s, 4H), 2.49 (s, 3H).

### Example 60. Synthesis of 5-(4-(3-fluoro-5-(trifluoromethyl) phenoxy)pyridin-2-yl)-N-(2-hydroxyethyl)-3-methyl-4-oxo-4,5,6,7-tetrahydropyrazolo[1,5-a]pyrazine-2-carboxamide (Compound 322)

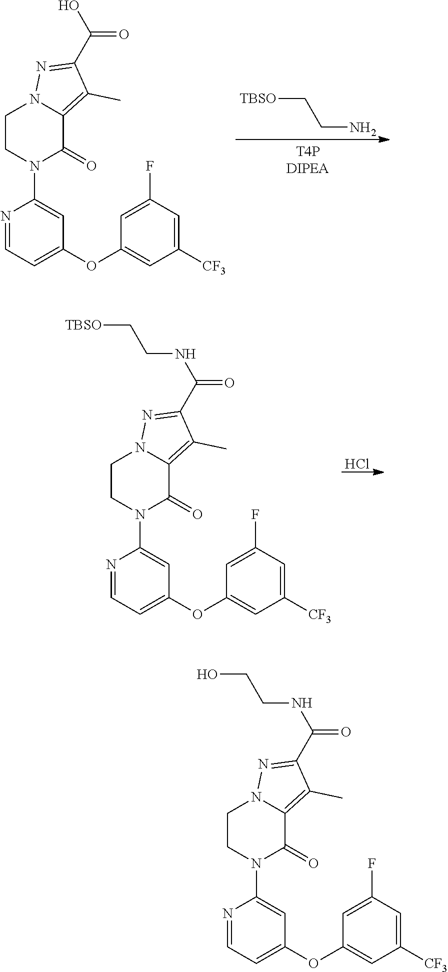 [SMILES: Cc1c(C(=O)O)nn2c1C(=O)N(c1cc(Oc3cc(F)cc(C(F)(F)F)c3)ccn1)CC2 ; CC(C)(C)[Si](C)(C)OCCN ; Cc1c(C(=O)NCCO[Si](C)(C)C(C)(C)C)nn2c1C(=O)N(c1cc(Oc3cc(F)cc(C(F)(F)F)c3)ccn1)CC2 ; Cl ; Cc1c(C(=O)NCCO)nn2c1C(=O)N(c1cc(Oc3cc(F)cc(C(F)(F)F)c3)ccn1)CC2]

Step A: To a solution of 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxylic acid (see, Example 59) and 2-((tert-butyldimethylsilyl)oxy) ethan-1-amine (31.15 mg, 177.64 μmol, 2 eq.) in DMF (2 mL) was added DIPEA (57.40 mg, 444.11 μmol, 77.35 μL, 5 eq.) and n-butylphosphoric anhydride (T4P, 96.00 mg, 266.46 μmol, 3 eq.). The mixture was stirred at 80° C. for 1 h. The reaction mixture was filtrated and concentrated under vacuum. The residue was purified by flash silica gel chromatography (hexane to hexane/EtOAc=70/30, TLC: hexane/EtOAc=2/1, R_{f}=0.5) to yield N-(2-((tert-butyldimethylsilyl)oxy)ethyl)-5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxamide (70 mg, 103.67 μmol, 58.36% yield) as a white solid. LCMS [M+H]⁺=608.3; ¹H NMR (400 MHz, DMSO-d₆) δ=8.44 (d, J=5.6 Hz, 1H), 8.04 (br t, J=5.6 Hz, 1H), 7.66 (br d, J=8.0 Hz, 1H), 7.61 (d, J=2.0 Hz, 1H), 7.56 (br d, J=9.6 Hz, 1H), 7.51 (s, 1H), 6.97 (dd, J=2.4, 5.6 Hz, 1H), 4.48 (s, 4H), 3.66 (t, J=6.8 Hz, 2H), 2.48 (s, 2H), 0.87-0.86 (m, 9H), 0.06-0.03 (m, 6H).

Step B: A mixture of N-(2-((tert-butyldimethylsilyl)oxy)ethyl)-5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxamide (100 mg, 164.56 μmol, 1 eq.) in HCl in dioxane (1 mL) was stirred at 20° C. for 1 h. The reaction mixture was diluted with H₂O (10 mL) and extracted with DCM (10 mL×3). The reaction mixture was concentrated under vacuum. The residue was purified by prep-HPLC (column: 55-Boston Prime C18 150×30 mm, 5 μm; mobile phase: [H₂O (0.05% NH₃H₂O+10 mM NH₄HCO₃)-ACN]; gradient: 41%-71% ACN over 10.0 min) to yield 5-(4-(3-fluoro-5-(trifluoromethyl) phenoxy)pyridin-2-yl)-N-(2-hydroxyethyl)-3-methyl-4-oxo-4,5,6,7-tetrahydropyrazolo[1,5-a]pyrazine-2-carboxamide (25.12 mg, 50.74 μmol, 30.84% yield) as a white solid. LCMS [M+H]⁺=494.1; ¹H NMR (400 MHz, DMSO-d₆) δ=8.45 (d, J=5.6 Hz, 1H), 8.03 (t, J=5.6 Hz, 1H), 7.67 (br d, J=8.4 Hz, 1H), 7.62 (d, J=2.4 Hz, 1H), 7.60-7.54 (m, 1H), 7.52 (s, 1H), 6.98 (dd, J=2.4, 5.6 Hz, 1H), 4.75 (t, J=5.6 Hz, 1H), 4.50 (s, 4H), 3.49 (q, J=6.0 Hz, 2H), 3.33-3.28 (m, 2H), 2.49 (s, 3H).

### Example 61. Synthesis of 3-(1,2-dihydroxyethyl)-7-(4-(3-fluoro-5-(trifluoromethyl) phenoxy)pyridin-2-yl)-1-methyl-6,7-dihydroimidazo[1,5-a]pyrazin-8(5H)-one (Compound 325)

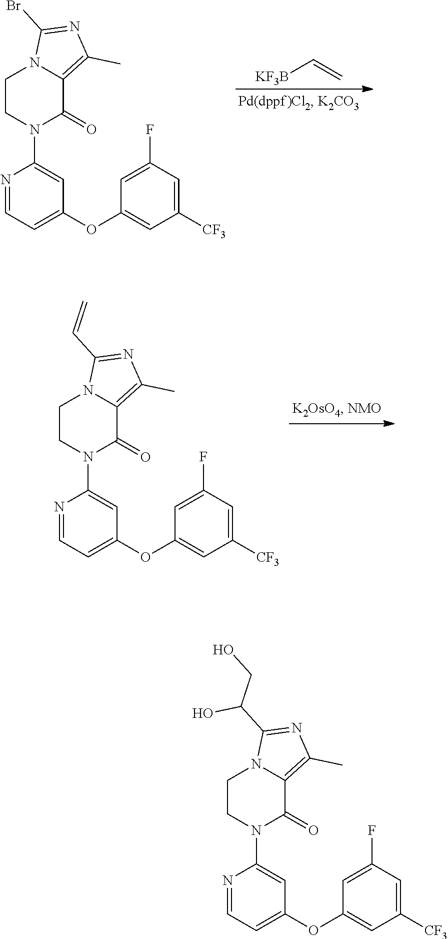

Step A: To a solution of 3-bromo-7-(4-(3-fluoro-5-(trifluoromethyl) phenoxy)pyridin-2-yl)-1-methyl-6,7-dihydroimidazo[1,5-a]pyrazin-8(5H)-one (500 mg, 1.03 mmol, 1 eq.) in dioxane (5 mL) and H₂O (0.5 mL) was added potassium vinyltrifluoroborate (276.05 mg, 2.06 mmol, 2 eq.), Pd(dppf)Cl₂ (75.40 mg, 103.04 μmol, 0.1 eq.) and K₂CO₃ (356.03 mg, 2.58 mmol, 2.5 eq.). The mixture was stirred at 90° C. for 1 h under N₂. The reaction mixture was concentrated under reduced pressure to give a residue. The residue was purified by flash silica gel chromatography (hexane to hexane/EtOAc=50/50, TLC: hexane/EOAc=1/1, R_{f}=0.20) to give 7-(4-(3-fluoro-5-(trifluoromethyl) phenoxy)pyridin-2-yl)-1-methyl-3-vinyl-6,7-dihydroimidazo[1,5-a]pyrazin-8(5H)-one (310 mg, 716.98 μmol, 69.58% yield) as a white solid: ¹H NMR (400 MHz, CDCl₃) δ=8.33 (d, J=5.6 Hz, 1H), 7.80 (d, J=2.4 Hz, 1H), 7.21-7.15 (m, 2H), 7.06-6.94 (m, 1H), 6.71 (dd, J=2.0, 5.6 Hz, 1H), 6.64-6.51 (m, 1H), 6.41-6.27 (m, 1H), 5.64 (d, J=11.2 Hz, 1H), 4.60-4.45 (m, 2H), 4.31-4.20 (m, 2H), 2.59 (s, 3H).

Step B: To a solution of 7-(4-(3-fluoro-5-(trifluoromethyl) phenoxy)pyridin-2-yl)-1-methyl-3-vinyl-6,7-dihydroimidazo[1,5-a]pyrazin-8(5H)-one (250 mg, 578.21 μmol, 1 eq.) in t-BuOH (2 mL) and acetone (2 mL) was added 4-methylmorpholine N-oxide (101.61 mg, 867.31 μmol, 91.54 μL, 1.5 eq.) and potassium osmate (VI) dihydrate (106.52 mg, 289.10 μmol, 0.5 eq.). The mixture was stirred at 25° C. for 16 h. The reaction mixture was filtered and concentrated to give a residue. The residue was purified by prep-HPLC (column: 52-Welch Xtimate C18 150×30 mm, 5 μm; mobile phase: [H₂O (10 mM NH₄HCO₃)-ACN]; gradient: 40%-70% ACN over 11.0 min) to give 3-(1,2-dihydroxyethyl)-7-(4-(3-fluoro-5-(trifluoromethyl) phenoxy)pyridin-2-yl)-1-methyl-6,7-dihydroimidazo[1,5-a]pyrazin-8(5H)-one (115.8 mg, 246.38 μmol, 42.61% yield) as a white solid. LCMS [M+H]⁺=467.2; ¹H NMR (400 MHz, DMSO-d₆) δ=8.40 (d, J=5.6 Hz, 1H), 7.71-7.61 (m, 2H), 7.55 (d, J=9.6 Hz, 1H), 7.49 (s, 1H), 6.92 (dd, J=2.0, 5.6 Hz, 1H), 5.65 (d, J=5.2 Hz, 1H), 4.85 (t, J=6.0 Hz, 1H), 4.74-4.62 (m, 1H), 4.49-4.30 (m, 4H), 3.78-3.60 (m, 2H), 2.36 (s, 3H).

### Example 62. Synthesis of 7-(4-(3-fluoro-5-(trifluoromethyl) phenoxy)pyridin-2-yl)-1-(hydroxymethyl)-6,7-dihydroimidazo[1,5-a]pyrazin-8(5H)-one (Compound 329)

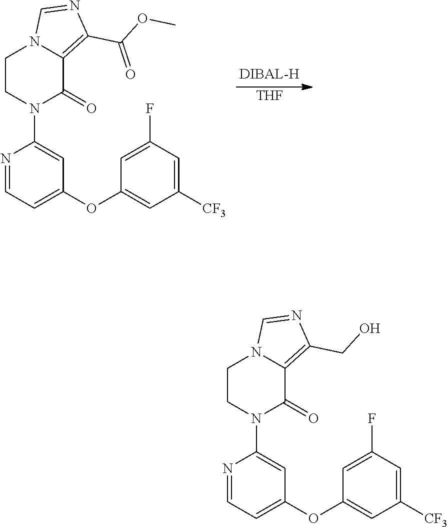 [SMILES: COC(=O)c1ncn2c1C(=O)N(c1cc(Oc3cc(F)cc(C(F)(F)F)c3)ccn1)CC2 ; O=C1c2c(CO)ncn2CCN1c1cc(Oc2cc(F)cc(C(F)(F)F)c2)ccn1 ; "THF"]

Step A: To a solution of methyl 7-(4-(3-fluoro-5-(trifluoromethyl) phenoxy)pyridin-2-yl)-8-oxo-5,6,7,8-tetrahydroimidazo[1,5-a]pyrazine-1-carboxylate (220 mg, 488.52 μmol, 1 eq.) in THF (15 mL) was added DIBAL-H (1 M, 1.47 mL, 3 eq.) at 0° C. The mixture was stirred at 0° C. for 1 h. The reaction mixture was quenched by addition H₂O (10 mL), and extracted with EtOAc (30 mL). The combined organic layers were washed with brine (30 mL), dried over sodium sulfate, filtered, and concentrated under reduced pressure to give a residue. The residue was purified by flash silica gel chromatography (DCM to DCM/MeOH =90/10, TLC: DCM/MeOH=8/1, R_{f}=0.3) to give the crude product. The crude product recrystallized from EtOH (2 mL) to give 7-(4-(3-fluoro-5-(trifluoromethyl) phenoxy)pyridin-2-yl)-1-(hydroxymethyl)-6,7-dihydroimidazo[1,5-a]pyrazin-8(5H)-one (81.81 mg, 193.71 μmol, 39.65% yield) as a white solid. LCMS [M+H]⁺=423.0; ¹H NMR (400 MHz, DMSO-d₆) δ=8.42 (d, J=5.6 Hz, 1H), 7.87 (s, 1H), 7.65 (d, J=8.4 Hz, 1H), 7.59 (d, J=2.4 Hz, 1H), 7.57-7.54 (m, 1H), 7.50 (s, 1H), 6.95 (dd, J=2.0, 5.6 Hz, 1H), 4.91 (t, J=6.0 Hz, 1H), 4.60 (d, J=6.0 Hz, 2H), 4.37 (s, 4H).

### Example 63. Synthesis of 5-(4-(3-fluoro-5-(trifluoromethyl) phenoxy)pyridin-2-yl)-4-oxo-4,5,6,7-tetrahydropyrazolo[1,5-a]pyrazine-2-carboxamide (Compound 330)

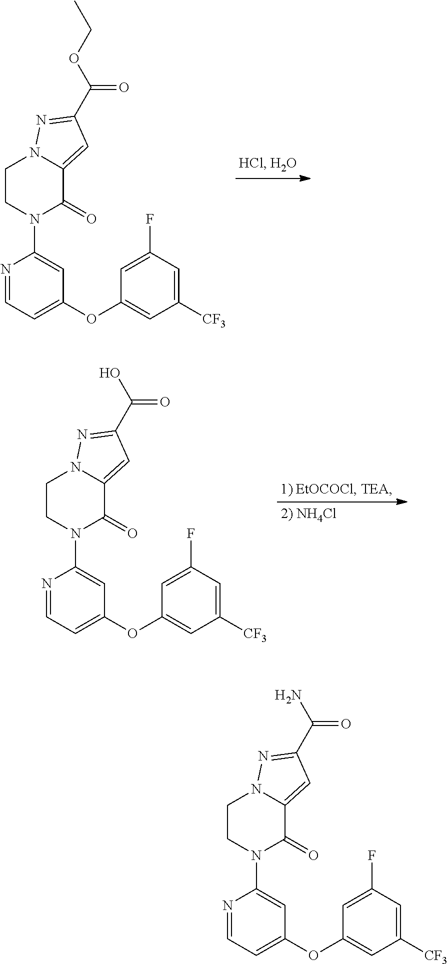 [SMILES: CCOC(=O)c1cc2n(n1)CCN(c1cc(Oc3cc(F)cc(C(F)(F)F)c3)ccn1)C2=O ; O=C(O)c1cc2n(n1)CCN(c1cc(Oc3cc(F)cc(C(F)(F)F)c3)ccn1)C2=O ; NC(=O)c1cc2n(n1)CCN(c1cc(Oc3cc(F)cc(C(F)(F)F)c3)ccn1)C2=O]

Step A: To a solution of ethyl 5-(4-(3-fluoro-5-(trifluoromethyl) phenoxy)pyridin-2-yl)-4-oxo-4,5,6,7-tetrahydropyrazolo[1,5-a]pyrazine-2-carboxylate (400 mg, 861.38 μmol, 1 eq.) in HCl (6M, 10 mL). The mixture was stirred at 80° C. for 1 h. The reaction mixture was concentrated to give 5-(4-(3-fluoro-5-(trifluoromethyl) phenoxy)pyridin-2-yl)-4-oxo-4,5,6,7-tetrahydropyrazolo[1,5-a]pyrazine-2-carboxylic acid (350 mg, crude) as a white solid, which was used into the next step without further purification. LCMS [M+H]⁺=437.3; ¹H NMR (400 MHz, DMSO-d₆) δ=8.45 (d, J=5.6 Hz, 1H), 7.67 (d, J=8.8 Hz, 1H), 7.62 (d, J=2.0 Hz, 1H), 7.58 (d, J=9.6 Hz, 1H), 7.52 (s, 1H), 7.22 (s, 1H), 7.00 (dd, J=2.0, 5.6 Hz, 1H), 4.59-4.53 (m, 4H).

Step B: To a solution of 5-(4-(3-fluoro-5-(trifluoromethyl) phenoxy)pyridin-2-yl)-4-oxo-4,5,6,7-tetrahydropyrazolo[1,5-a]pyrazine-2-carboxylic acid (300 mg, 687.57 μmol, 1 eq.) in THF (20 mL) and DCM (2 mL) was added TEA (208.73 mg, 2.06 mmol, 287.10 μL, 3 eq.) and ethyl chloroformate (0.37 g, 3.41 mmol, 325.99 μL, 4.96 eq.) at 0° C. was stirred for 30 min. Then NH₄Cl (1 M, 1.72 mL, 2.5 eq.) was added. The mixture was stirred at 0° C. for another 30 min. The reaction mixture was quenched by addition H₂O (20 mL) at 0° C., and extracted with EtOAc (50 mL). The combined organic layers were washed with brine (20 mL), dried over sodium sulfate, filtered, and concentrated under reduced pressure to give a residue. The residue was triturated with EtOAc (10 mL) at 25° C. for 1 h, filtered, and filter cake was dried under vacuum to give the crude product. The crude product recrystallized from EtOH (10 mL) and EtOAc (10 mL) to give 5-(4-(3-fluoro-5-(trifluoromethyl) phenoxy)pyridin-2-yl)-4-oxo-4,5,6,7-tetrahydropyrazolo[1,5-a]pyrazine-2-carboxamide (177.58 mg, 407.92 μmol, 59.33% yield) as a white solid. LCMS [M+H]⁺=436.1; ¹H NMR (400 MHz, DMSO-d₆) δ=8.45 (d, J=6.0 Hz, 1H), 7.74 (s, 1H), 7.67 (br d, J=8.8 Hz, 1H), 7.63 (d, J=2.0 Hz, 1H), 7.59-7.56 (m, 1H), 7.52 (s, 1H), 7.41 (s, 1H), 7.20 (s, 1H), 6.99 (dd, J=2.4, 6.0 Hz, 1H), 4.55 (s, 4H).

### Example 64. Synthesis of 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carbonitrile (Compound 335)

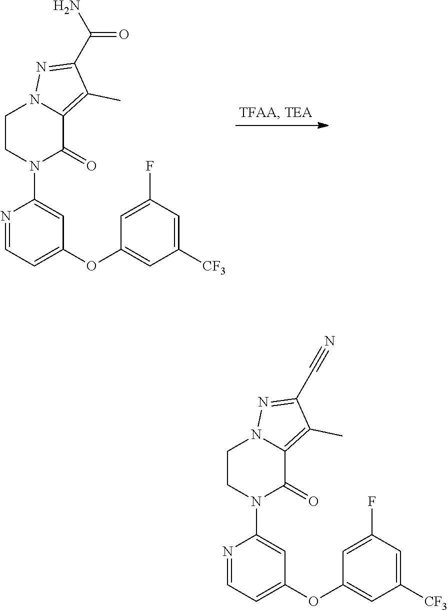 [SMILES: Cc1c(C(N)=O)nn2c1C(=O)N(c1cc(Oc3cc(F)cc(C(F)(F)F)c3)ccn1)CC2 ; Cc1c(C#N)nn2c1C(=O)N(c1cc(Oc3cc(F)cc(C(F)(F)F)c3)ccn1)CC2]

Step A: To a solution of 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carboxamide (200 mg, 445.08 μmol, 1 eq.) in DCM (10 mL) was added TEA (157.63 mg, 1.56 mmol, 216.82 μL, 3.5 eq.) was stirred at 25° C. for 30 min. Trifluoroacetic anhydride (TFAA, 327.18 mg, 1.56 mmol, 216.53 μL, 3.5 eq.) was added. The mixture was stirred at 25° C. for 3 h. The reaction mixture was concentrated under reduced pressure to remove DCM. The residue was diluted with H₂O (20 mL) and extracted with EtOAc (40 mL). The combined organic layers were washed with brine, dried over, filtered and concentrated under reduced pressure to give a residue. The residue was triturated with MTBE (10 mL) at 25° C. for 1 h, filtered and filter cake was concentrated under reduced pressure to give 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carbonitrile (177 mg, 410.35 μmol, 92.20% yield) as a white solid. LCMS [M+H]⁺=432.2; ¹H NMR (400 MHz, DMSO-d₆) δ=8.45 (d, J=5.6 Hz, 1H), 7.67 (d, J=8.4 Hz, 1H), 7.61-7.54 (m, 2H), 7.52 (s, 1H), 7.00 (dd, J=2.4, 5.6 Hz, 1H), 4.58-4.51 (m, 4H), 2.36 (s, 3H).

### Example 65. Synthesis of 2-(aminomethyl)-5-(4-(3-fluoro-5-(trifluoromethyl) phenoxy)pyridin-2-yl)-3-methyl-6,7-dihydropyrazolo[1,5-a]pyrazin-4(5H)-one (Compound 341)

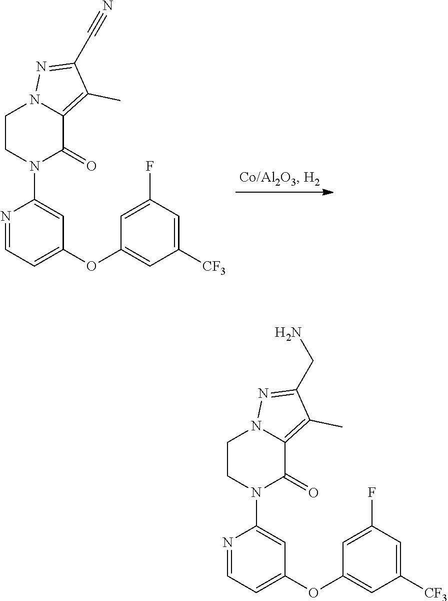 [SMILES: Cc1c(C#N)nn2c1C(=O)N(c1cc(Oc3cc(F)cc(C(F)(F)F)c3)ccn1)CC2 ; Cc1c(CN)nn2c1C(=O)N(c1cc(Oc3cc(F)cc(C(F)(F)F)c3)ccn1)CC2]

Step A: A solution of 5-(4-(3-fluoro-5-(trifluoromethyl) phenoxy)pyridin-2-yl)-3-methyl-4-oxo-4,5,6,7-tetrahydropyrazolo[1,5-a]pyrazine-2-carbonitrile (0.1 g, 231.83 μmol, 1 eq.) was dissolved in NH₃ in MeOH (10 mL). This solution was delivered by a plunger pump at a flow rate of 0.3 mL/min. Hydrogen (467.33 μg, 231.83 μmol, 1 eq.) (from a high-pressure cylinder) was introduced via a mass flow controller (MFC) at 30 sccm (2 equiv) and the system was maintained at 2 MPa. The liquid and gas streams were mixed with a T-shaped mixer and the combined solution was passed into a stainless-steel (SS) fixed-bed reactor (Outer diameter: 6.350 mm (¼ inch); Internal volume: 5 mL) packed with 3.53 g Co/Al₂O₃ (7.65 mg, 231.83 μmol, 1 eq.) and held at 100° C. Upon complete injection of the reaction solution, the solvent feed was switched to MeOH at 10 mL/min and the reactor was flushed for 10 min. The reaction mixture was concentrated to give a residue. The mixture was purified by prep-HPLC (column: 52-Welch Xtimate C18 150×30 mm, 5 μm; mobile phase: [H₂O (10 mM NH₄HCO₃)-ACN]; gradient: 37%-67% ACN over 11.0 min) to give the residue. The residue was triturated with (PE/MTBE=5/1, 3 mL) at 25° C. for 1 h. filtered and filter cake was concentrated under reduced pressure to give 2-(aminomethyl)-5-(4-(3-fluoro-5-(trifluoromethyl) phenoxy)pyridin-2-yl)-3-methyl-6,7-dihydropyrazolo[1,5-a]pyrazin-4(5H)-one (23.65 mg, 54.32 μmol, 23.43% yield) as a white solid. LCMS [M+H]⁺=436.1; 1H NMR (400 MHz, CDCl₃) δ=8.43 (d, J=5.6 Hz, 1H), 7.66 (d, J=8.8, 1H), 7.62 (s, 1H), 7.56 (d, J=9.2, 1H), 7.50 (s, 1H), 6.99-6.92 (m, 1H), 4.48-4.32 (m, 4H), 3.66 (s, 2H), 2.23 (s, 3H).

### Example 66. Synthesis of 5-(4-(3-fluoro-5-(trifluoromethyl) phenoxy)pyridin-2-yl)-3-(hydroxymethyl)-4-oxo-4,5,6,7-tetrahydropyrazolo[1,5-a]pyrazine-2-carboxamide (Compound 345)

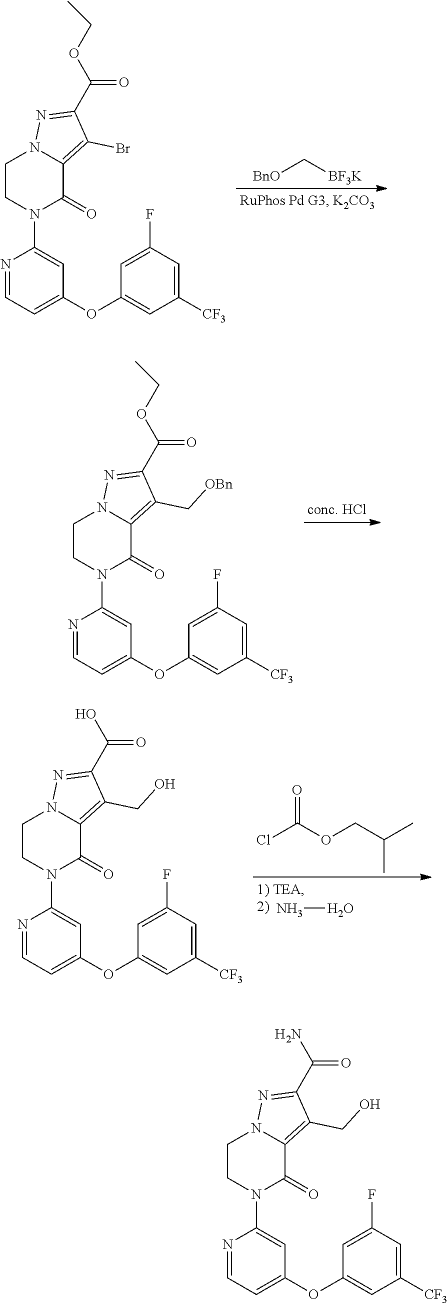

Step A: To a solution of ethyl 3-bromo-5-(4-(3-fluoro-5-(trifluoromethyl) phenoxy)pyridin-2-yl)-4-oxo-4,5,6,7-tetrahydropyrazolo[1,5-a]pyrazine-2-carboxylate (300 mg, 552.22 μmol, 1 eq.), potassium benzyloxymethyltrifluoroborate (377.82 mg, 1.66 mmol, 3 eq.) and K₂CO₃ (305.28 mg, 2.21 mmol, 4 eq.) in dioxane (20 mL) and H₂O (4 mL) was added RuPhos Pd G3 (92.37 mg, 110.44 μmol, 0.2 eq.) and the mixture was stirred at 100° C. for 4 h under N₂. The reaction mixture was added H₂O (40 mL) and then extracted with EtOAc (50 mL×3). The separated organic layers were washed with brine (50 mL×2), dried over Na₂SO₄, and filtered. The filtrate was concentrated under reduced pressure to give the crude product. The crude product was purified by flash silica gel chromatography (hexane to hexane/EtOAc=55/45) to give ethyl 3-((benzyloxy)methyl)-5-(4-(3-fluoro-5-(trifluoromethyl) phenoxy)pyridin-2-yl)-4-oxo-4,5,6,7-tetrahydropyrazolo[1,5-a]pyrazine-2-carboxylate (196 mg, 150.89 μmol, 27.33% yield) as a white solid. LCMS [M+H]⁺=585.3; ¹H NMR (400 MHz, DMSO-d₆) δ=8.45 (dd, J=2.4, 5.6 Hz, 1H), 7.69-7.55 (m, 4H), 7.33 (s, 5H), 7.00 (dd, J=2.4, 5.6 Hz, 1H), 4.90 (s, 1H), 4.63-4.52 (m, 5H), 4.50 (s, 1H), 4.30 (q, J=7.2 Hz, 3H), 1.17 (t, J=7.2 Hz, 3H).

Step B: To a solution of ethyl 3-((benzyloxy)methyl)-5-(4-(3-fluoro-5-(trifluoromethyl) phenoxy)pyridin-2-yl)-4-oxo-4,5,6,7-tetrahydropyrazolo[1,5-a]pyrazine-2-carboxylate (370 mg, 633.00 μmol, 1 eq.) in HCl (6 M, 9.74 mL, 92.29 eq.) and the mixture was stirred in the microwave at 80° C. for 4 h. The mixture was concentrated under reduced pressure to give a residue. The crude product was purified by prep-HPLC (column: Boston Green ODS 150*30 mm*5 μm; mobile phase: [H₂O (0.04% HCl)-ACN]; gradient: 43%-63% ACN over 11.0 min) to give 5-(4-(3-fluoro-5-(trifluoromethyl) phenoxy)pyridin-2-yl)-3-(hydroxymethyl)-4-oxo-4,5,6,7-tetrahydropyrazolo[1,5-a]pyrazine-2-carboxylic acid (76.6 mg, 175.50 μmol, 34.26% yield) as a white solid. LCMS [M+H]⁺=467.1; ¹H NMR (400 MHz, DMSO-d₆) δ=8.47 (d, J=5.6 Hz, 1H), 7.68 (d, J=8.4 Hz, 1H), 7.63-7.56 (m, 2H), 7.53 (s, 1H), 7.01 (dd, J=2.4, 5.6 Hz, 1H), 4.86 (s, 2H), 4.62-4.46 (m, 4H).

Step C: To a solution of 5-(4-(3-fluoro-5-(trifluoromethyl) phenoxy)pyridin-2-yl)-3-(hydroxymethyl)-4-oxo-4,5,6,7-tetrahydropyrazolo[1,5-a]pyrazine-2-carboxylic acid (160 mg, 343.10 μmol, 1 eq.) in THF (6 mL) was added TEA (138.87 mg, 1.37 mmol, 191.02 μL, 4 eq.) and isobutyl chloroformate (140.58 mg, 1.03 mmol, 134.65 μL, 3 eq.) at 0° C., the mixture was stirred at 0° C. for 30 min under N₂, then NH₃ H₂O (107.36 mg, 857.74 μmol, 117.97 μL, 28% purity, 2.5 eq.) was added to the mixture, the mixture was stirred at 0° C. for 2 h under N₂. The mixture was filtered. The filter cake was washed with EtOAc (20 mL×2), The filter liquor was concentrated under reduced pressure to give a residue. The crude product was purified by flash silica gel chromatography (DCM to DCM/MeOH=97/3) to give 5-(4-(3-fluoro-5-(trifluoromethyl) phenoxy)pyridin-2-yl)-3-(hydroxymethyl)-4-oxo-4,5,6,7-tetrahydropyrazolo[1,5-a]pyrazine-2-carboxamide (100 mg, 353.15 μmol, 54.90% yield) as a white solid. LCMS [M+H]⁺=466.1; ¹H NMR (400 MHz, DMSO-d₆) δ=8.45 (d, J=5.6 Hz, 1H), 8.02 (s, 1H), 7.73 (s, 1H), 7.66 (d, J=8.5 Hz, 1H), 7.61-7.55 (m, 2H), 7.52 (s, 1H), 6.99 (dd, J=2.4, 5.6 Hz, 1H), 5.52 (t, J=6.4 Hz, 1H), 4.84 (d, J=6.4 Hz, 2H), 4.58-4.46 (m, 4H).

### Example 67. Synthesis of 5-(4-(3-fluoro-5-(trifluoromethyl) phenoxy)pyridin-2-yl)-3-(methylamino)-4-oxo-4,5,6,7-tetrahydropyrazolo[1,5-a]pyrazine-2-carboxamide (Compound 351)

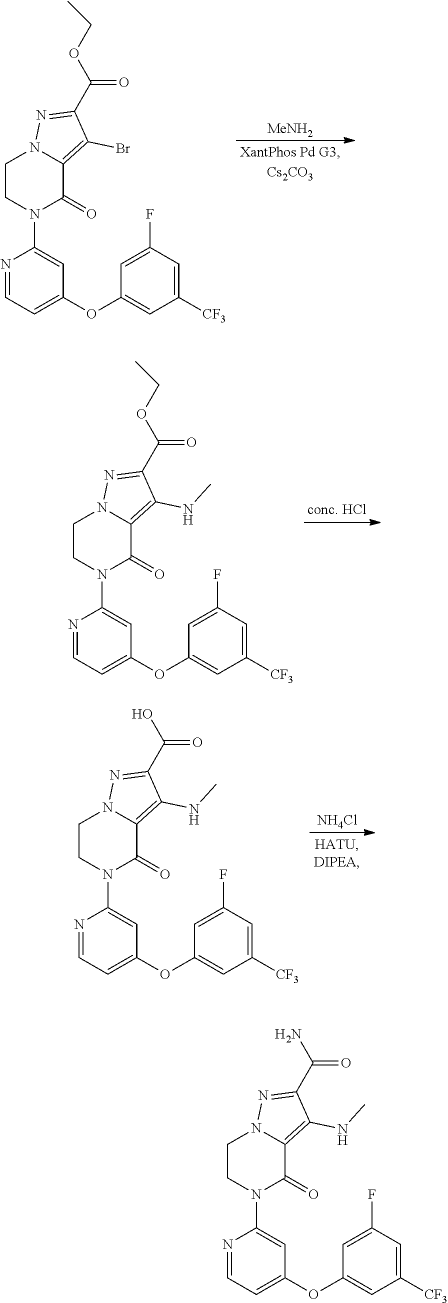

Step A: A mixture of ethyl 3-bromo-5-(4-(3-fluoro-5-(trifluoromethyl) phenoxy)pyridin-2-yl)-4-oxo-4,5,6,7-tetrahydropyrazolo[1,5-a]pyrazine-2-carboxylate (370 mg, 681.07 μmol, 1 eq.), methylamine hydrochloride (229.92 mg, 3.41 mmol, 5 eq.), XantPhos Pd G3 (64.59 mg, 68.11 μmol, 0.1 eq.) and Cs₂CO₃ (1.11 g, 3.41 mmol, 5 eq.) in dioxane (10 mL) was degassed and purged with N₂ three times, then the mixture was stirred at 100° C. for 2 h. The reaction mixture was filtered and concentrated under vacuum to dryness to give a crude product. The residue was purified by flash silica gel chromatography (hexanes to hexanes/EtOAc=60/40, TLC: hexanes/EtOAc=1/1, R_{f}=0.3) to give ethyl 5-(4-(3-fluoro-5-(trifluoromethyl) phenoxy)pyridin-2-yl)-3-(methylamino)-4-oxo-4,5,6,7-tetrahydropyrazolo[1,5-a]pyrazine-2-carboxylate (240 mg, 340.49 μmol, 49.99% yield) as a white solid. LCMS [M+H]⁺=494.2; ¹H NMR (400 MHz, DMSO-d₆) δ=8.43 (d, J=5.6 Hz, 1H), 7.66 (br d, J=8.8 Hz, 1H), 7.62 (d, J=2.0 Hz, 1H), 7.59-7.55 (m, 1H), 7.51 (s, 1H), 6.95 (dd, J=2.4, 5.6 Hz, 1H), 5.89-5.77 (m, 1H), 4.48-4.40 (m, 4H), 4.29 (d, J=7.2 Hz, 2H), 2.96 (d, J=5.6 Hz, 3H), 1.32-1.28 (m, 3H).

Step B: A mixture of ethyl 5-(4-(3-fluoro-5-(trifluoromethyl) phenoxy)pyridin-2-yl)-3-(methylamino)-4-oxo-4,5,6,7-tetrahydropyrazolo[1,5-a]pyrazine-2-carboxylate (220 mg, 445.88 μmol, 1 eq.) in HCl (6 M, 5 mL, 67.28 eq.) was stirred at 80° C. for 2 h. The mixture was concentrated under reduced pressure to give 5-(4-(3-fluoro-5-(trifluoromethyl) phenoxy)pyridin-2-yl)-3-(methylamino)-4-oxo-4,5,6,7-tetrahydropyrazolo[1,5-a]pyrazine-2-carboxylic acid (200 mg, crude) as a white solid, which was used in the next step without further purification. LCMS [M+H]⁺=466.1; ¹H NMR (400 MHz, DMSO-d₆) δ=8.42 (d, J=5.6 Hz, 1H), 7.70-7.59 (m, 3H), 7.59-7.54 (m, 1H), 7.51 (s, 1H), 6.97-6.92 (m, 1H), 4.46-4.41 (m, 4H), 2.96 (s, 3H).

Step C: To a solution of 5-(4-(3-fluoro-5-(trifluoromethyl) phenoxy)pyridin-2-yl)-3-(methylamino)-4-oxo-4,5,6,7-tetrahydropyrazolo[1,5-a]pyrazine-2-carboxylic acid (210 mg, 451.27 μmol, 1 eq.) and NH₄Cl (120.69 mg, 2.26 mmol, 5 eq.) in DMF (3 mL) was added HATU (343.17 mg, 902.53 μmol, 2 eq.) and DIEA (291.61 mg, 2.26 mmol, 393.01 μL, 5 eq.). The mixture was stirred at 20° C. for 1 h. The mixture was concentrated under vacuum to give a residue. The residue was purified by prep-HPLC (column: 52-Welch Xtimate C18 150×30 mm, 5 μm; mobile phase: [H₂O (10 mM NH₄HCO₃)-ACN]; gradient: 45%-75% ACN over 11.0 min) to give 5-(4-(3-fluoro-5-(trifluoromethyl) phenoxy)pyridin-2-yl)-3-(methylamino)-4-oxo-4,5,6,7-tetrahydropyrazolo[1,5-a]pyrazine-2-carboxamide (65 mg, 137.29 μmol, 31.88% yield) as a white solid. LCMS [M+H]⁺=465.1; ¹H NMR (400 MHz, DMSO-d₆) δ=8.42 (d, J=6.0 Hz, 1H), 7.68-7.60 (m, 2H), 7.59-7.53 (m, 2H), 7.51 (s, 1H), 7.29 (br s, 1H), 6.93 (dd, J=2.0, 5.6 Hz, 1H), 6.24 (q, J=5.2 Hz, 1H), 4.49-4.34 (m, 4H), 2.93 (d, J=5.6 Hz, 3H).

### Example 68. Synthesis of 6-(4-(3-fluoro-5-(trifluoromethyl) phenoxy)pyridin-2-yl)-1-methyl-7-oxo-4,5,6,7-tetrahydro-1H-pyrazolo[3,4-c]pyridine-3-carboxamide (Compound 357)

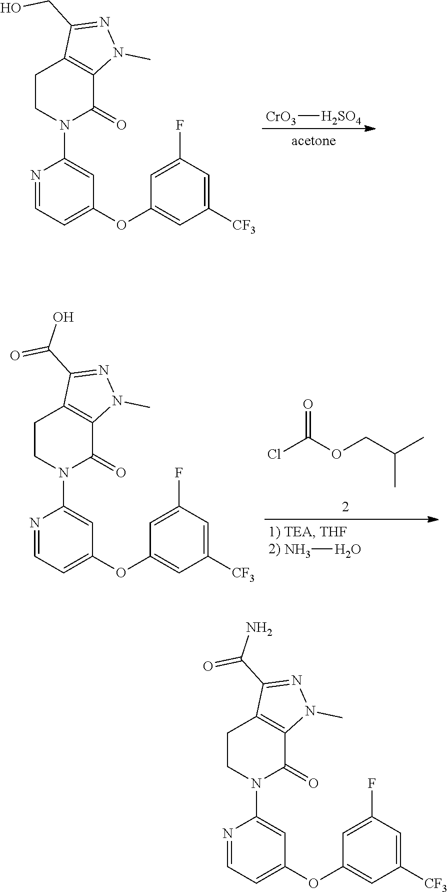

Step A: To a solution of 6-(4-(3-fluoro-5-(trifluoromethyl) phenoxy)pyridin-2-yl)-3-(hydroxymethyl)-1-methyl-1,4,5,6-tetrahydro-7H-pyrazolo[3,4-c]pyridin-7-one (35 mg, 80.21 μmol, 1 eq.) in acetone (2 mL) was added Jones reagent (0.83 M, 0.3 mL, 3.10 eq.) at 0° C., and then the mixture was stirred at 20° C. for 16 h. The reaction mixture was cooled to 0° C., where a large amount of yellow solid precipitated. The solid was filtered and filter cake dried under vacuum to yield 6-(4-(3-fluoro-5-(trifluoromethyl) phenoxy)pyridin-2-yl)-1-methyl-7-oxo-4,5,6,7-tetrahydro-1H-pyrazolo[3,4-c]pyridine-3-carboxylic acid (40 mg, crude) as a yellow solid, which was used in the next step without further purification. LCMS [M+H]⁺=451.2.

Step B: To a solution of 6-(4-(3-fluoro-5-(trifluoromethyl) phenoxy)pyridin-2-yl)-1-methyl-7-oxo-4,5,6,7-tetrahydro-1H-pyrazolo[3,4-c]pyridine-3-carboxylic aci (40 mg, 88.82 μmol, 1 eq.) in THF (1.5 mL) was added TEA (35.95 mg, 355.29 μmol, 49.45 μL, 4 eq.) and isobutyl chloroformate (36.39 mg, 266.46 μmol, 34.86 μL, 3 eq.) at 0° C. The mixture was stirred at 0° C. for 0.5 h. Then NH₃ H₂O (25.94 mg, 222.05 μmol, 28.51 μL, 30% purity, 2.5 eq.) was added to the mixture. The mixture was stirred at 0° C. for 0.5 h. Water was added and the reaction was extracted with DCM (10 mL×3). The combined organic layers were dried over Na₂SO₄, filtered, and concentrated under vacuum to give a residue. The residue was purified by prep-HPLC (column: 52-Welch Xtimate C18 150×30 mm, 5 μm; mobile phase: [H₂O (10 mM NH₄HCO₃)-ACN]; gradient: 43%-73% ACN over 11.0 min) to give 6-(4-(3-fluoro-5-(trifluoromethyl) phenoxy)pyridin-2-yl)-1-methyl-7-oxo-4,5,6,7-tetrahydro-1H-pyrazolo[3,4-c]pyridine-3-carboxamide (18 mg, 40.06 μmol, 45.00% yield) as a white solid. LCMS [M+H]⁺=450.0; ¹H NMR (400 MHz, DMSO-d₆) δ=8.42 (d, J=5.6 Hz, 1H), 7.65 (br d, J=8.4 Hz, 1H), 7.60 (s, 1H), 7.58-7.53 (m, 1H), 7.52-7.46 (m, 2H), 7.37 (s, 1H), 6.96 (dd, J=2.4, 5.6 Hz, 1H), 4.26 (t, J=6.4 Hz, 2H), 4.11 (s, 3H), 3.09 (t, J=5.6 Hz, 2H).

### Example 69. Synthesis of 5-(4-(3-fluoro-5-(trifluoromethyl) phenoxy)pyridin-2-yl)-3-methyl-4-oxo-4,5,6,7-tetrahydropyrazolo[1,5-a]pyrazine-2-sulfinamide (Compound 365)

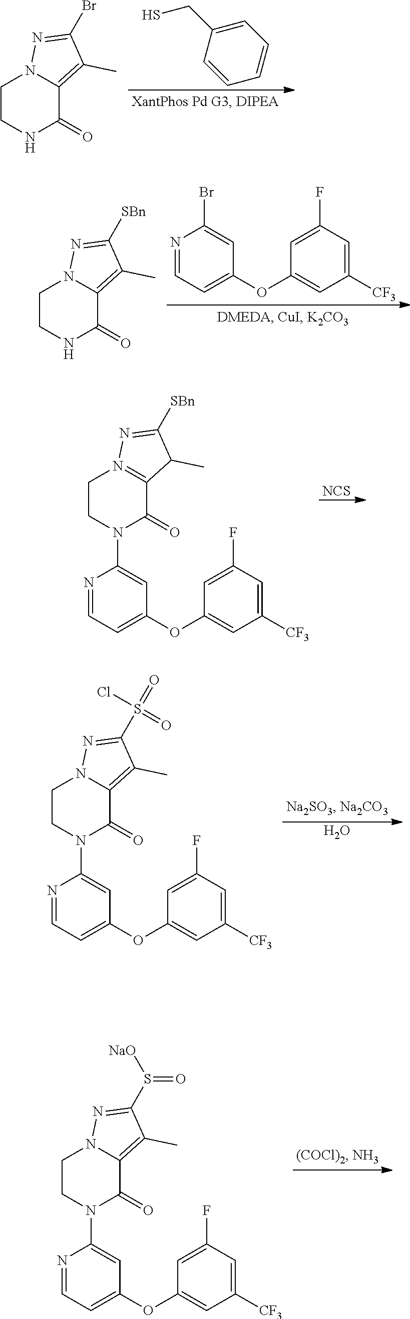

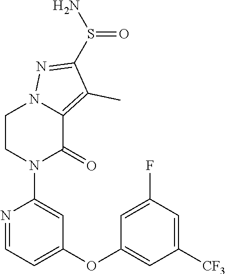 [SMILES: Cc1c(S(N)=O)nn2c1C(=O)N(c1cc(Oc3cc(F)cc(C(F)(F)F)c3)ccn1)CC2]

Step A: To a solution of 2-bromo-3-methyl-6,7-dihydropyrazolo[1,5-a]pyrazin-4(5H)-one (1 g, 4.35 mmol, 1 eq.), benzyl mercaptan (720 mg, 5.80 mmol, 680.53 μL, 1.33 eq.), DIEA (1.12 g, 8.69 mmol, 1.51 mL, 2 eq.) in dioxane (20 mL) was added XantPhos Pd G3 (412.22 mg, 434.67 μmol, 0.1 eq.). The mixture was stirred at 120° C. for 16 h under N₂ atmosphere. Water (40 mL) was added and then the reaction was extracted with EtOAc (50 mL×3). The separated organic layers were washed with brine (50 mL×2), dried over Na₂SO₄, and filtered. The filtrate was concentrated under reduced pressure to give the crude product. The crude product was purified by flash silica gel chromatography (hexane to hexane/EtOAc=27/73, EA=1, Rf-0.5) to give a yellow solid. The yellow solid was purified by reversed-phase silica gel chromatography (H₂O to H₂O/MeCN=44/56) to give 2-(benzylthio)-3-methyl-6,7-dihydropyrazolo[1,5-a]pyrazin-4(5H)-one (550 mg, 1.91 mmol, 43.98% yield) as a white solid. LCMS [M+H]⁺=274.1; ¹H NMR (400 MHz, DMSO-d₆) δ=8.13 (s, 1H), 7.39-7.16 (m, 5H), 4.28-4.19 (m, 2H), 4.06 (s, 2H), 3.59-3.51 (m, 2H), 2.01 (s, 3H).

Step B: To a solution of 2-(benzylthio)-3-methyl-6,7-dihydropyrazolo[1,5-a]pyrazin-4(5H)-one (550 mg, 2.01 mmol, 1 eq.), 2-bromo-4-(3-fluoro-5-(trifluoromethyl) phenoxy)pyridine (743.83 mg, 2.21 mmol, 1.1 eq.), K₃PO₄ (1.28 g, 6.04 mmol, 3 eq.) and CuI (76.64 mg, 402.41 μmol, 0.2 eq.) in dioxane (16 mL) was added DMEDA (35.47 mg, 402.41 μmol, 43.31 μL, 0.2 eq.). The mixture was stirred at 100° C. for 2 h under N₂ atmosphere. Water (30 mL) was added and then the reaction was extracted with EtOAc (30 mL×3). The separated organic layers were washed with brine (50 mL×2), dried over Na₂SO₄, and filtered. The filtrate was concentrated under vacuum to give the crude product. The crude product was purified by flash silica gel chromatography (hexane to hexane/EtOAc=60/40) to give 2-(benzylthio)-5-(4-(3-fluoro-5-(trifluoromethyl) phenoxy)pyridin-2-yl)-3-methyl-6,7-dihydropyrazolo[1,5-a]pyrazin-4(5H)-one (950 mg, 1.62 mmol, 80.40% yield) as a white solid. LCMS [M+H]⁺=529.3; ¹H NMR (400 MHz, DMSO-d₆) δ=88.43 (d, J=5.6 Hz, 1H), 7.65 (d, J=8.4 Hz, 1H), 7.61 (d, J=2.4 Hz, 1H), 7.58-7.53 (m, 1H), 7.50 (s, 1H), 7.31-7.20 (m, 5H), 6.96 (dd, J=2.4, 5.6 Hz, 1H), 4.45 (s, 4H), 4.09 (s, 2H), 2.05 (s, 3H).

Step C: To a solution of 2-(benzylthio)-5-(4-(3-fluoro-5-(trifluoromethyl) phenoxy)pyridin-2-yl)-3-methyl-6,7-dihydropyrazolo[1,5-a]pyrazin-4(5H)-one (950 mg, 1.80 mmol, 1 eq.) in AcOH (12 mL) and H₂O (1.2 mL) was added NCS (960.09 mg, 7.19 mmol, 4 eq.) at 0° C. The mixture was stirred at 20° C. for 2 h. This reaction was concentrated under vacuum to give a residue. The crude product was purified by flash silica gel chromatography (hexane to hexane/EtOAc=70/30) to give 5-(4-(3-fluoro-5-(trifluoromethyl) phenoxy)pyridin-2-yl)-3-methyl-4-oxo-4,5,6,7-tetrahydropyrazolo[1,5-a]pyrazine-2-sulfonyl chloride (690 mg, 1.23 mmol, 68.43% yield) as a white solid. LCMS [M+H]⁺=505.2; ¹H NMR (400 MHz, DMSO-d₆) δ=8.44 (d, J=5.6 Hz, 1H), 7.68-7.62 (m, 2H), 7.59-7.53 (m, 1H), 7.51 (s, 1H), 6.97 (dd, J=2.4, 5.6 Hz, 1H), 4.48-4.35 (m, 4H), 2.34 (s, 3H).

Step D: A solution of Na₂SO₃ (1.03 g, 8.20 mmol, 6 eq.) in H₂O (16 mL) was stirred at 20° C. for 10 min. Then Na₂CO₃ (869.17 mg, 8.20 mmol, 6 eq.) was added the above solution at 20° C. The mixture was stirred at 50° C. for 10 min. Then 5-(4-(3-fluoro-5-(trifluoromethyl) phenoxy)pyridin-2-yl)-3-methyl-4-oxo-4,5,6,7-tetrahydropyrazolo[1,5-a]pyrazine-2-sulfonyl chloride (690 mg, 1.37 mmol, 1 eq.) was added the above solution at 20° C. The mixture was stirred at 60° C. for 16 h. The mixture was filtered. The filter cake was concentrated under reduced pressure to give sodium 5-(4-(3-fluoro-5-(trifluoromethyl) phenoxy)pyridin-2-yl)-3-methyl-4-oxo-4,5,6,7-tetrahydropyrazolo[1,5-a]pyrazine-2-sulfinate (600 mg, crude) as a white solid. The crude product was used directly in the next step without further purification. LCMS [M+H]⁺=471.2; ¹H NMR (400 MHz, DMSO-d₆) δ=8.47-8.41 (m, 1H), 7.70-7.61 (m, 2H), 7.56 (d, J=9.4 Hz, 1H), 7.50 (s, 1H), 7.01-6.93 (m, 1H), 4.56-4.33 (m, 4H), 2.47-2.30 (m, 3H).

Step E: To a solution of sodium 5-(4-(3-fluoro-5-(trifluoromethyl) phenoxy)pyridin-2-yl)-3-methyl-4-oxo-4,5,6,7-tetrahydropyrazolo[1,5-a]pyrazine-2-sulfinate (300 mg, 609.29 μmol, 1 eq.) in THF (8 mL) was added oxalyl chloride (773.37 mg, 6.09 mmol, 533.36 μL, 10 eq.) at 0° C. The reaction mixture was stirred at 20° C. for 1 h. The mixture was concentrated under reduced pressure to give the crude product. The crude product in DCM (8 mL) was added dropwise a solution of NH₃ in THF at 0° C., which had been prepared by bubbling NH₃ gas (15 psi) into THF (10 mL) at −70° C. for 30 min. The resulting mixture was stirred at 20° C. for 1 h. Water (40 mL) was added and the reaction was extracted with EtOAc (40 mL×3). The separated organic layers were washed with brine (50 mL×2), dried over Na₂SO₄, and filtered. The filtrate was concentrated under reduced pressure to give the crude product. The crude product was purified by prep-HPLC (column: 52-Welch Xtimate C18 150×30 mm, 5 μm; mobile phase: [H₂O (10 mM NH₄HCO₃)-ACN]; gradient: 40%-70% ACN over 11.0 min) to give 5-(4-(3-fluoro-5-(trifluoromethyl) phenoxy)pyridin-2-yl)-3-methyl-4-oxo-4,5,6,7-tetrahydropyrazolo[1,5-a]pyrazine-2-sulfinamide (65 mg, 138.47 μmol, 22.73% yield) as a white solid. LCMS [M+H]⁺=470.1; ¹H NMR (400 MHz, DMSO-d₆) δ=8.44 (d, J=5.6 Hz, 1H), 7.67 (d, J=8.4 Hz, 1H), 7.61 (d, J=2.0 Hz, 1H), 7.57 (d, J=9.6 Hz, 1H), 7.51 (s, 1H), 6.98 (dd, J=2.4, 5.6 Hz, 1H), 6.42 (s, 2H), 4.49 (s, 4H), 2.40 (s, 3H).

### Example 70. Synthesis of 7-(4-(3-fluoro-5-(methylamino) phenoxy)pyridin-2-yl)-3-(2-hydroxyethyl)-1-methyl-6,7-dihydroimidazo[1,5-a]pyrazin-8(5H)-one (Compound 366)

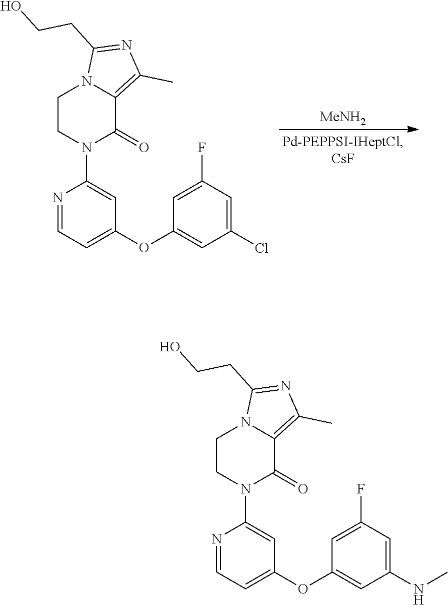 [SMILES: "MeNH2" ; Cc1nc(CCO)n2c1C(=O)N(c1cc(Oc3cc(F)cc(Cl)c3)ccn1)CC2 ; CNc1cc(F)cc(Oc2ccnc(N3CCn4c(CCO)nc(C)c4C3=O)c2)c1]

Step A: To a solution of 7-(4-(3-chloro-5-fluorophenoxy)pyridin-2-yl)-3-(2-hydroxyethyl)-1-methyl-6,7-dihydroimidazo[1,5-a]pyrazin-8(5H)-one (120 mg, 287.89 μmol, 1 eq.) and methylamine hydrochloride (116.62 mg, 1.73 mmol, 6 eq.) in DME (3 mL) was added Pd-PEPPSI-IHeptCl (CAS #1814936-54-3, 28.00 mg, 28.79 μmol, 0.1 eq.) and CsF (349.85 mg, 2.30 mmol, 8 eq.) at 20° C. under N₂ atmosphere. Then the reaction mixture was stirred at 90° C. for 16 h under a N₂ atmosphere. Then, the reaction mixture was diluted with H₂O (15 mL) and extracted with EtOAc (15 mL×3). The combined organic layers were dried over Na₂SO₄, filtered and concentrated under vacuum to give the crude product. The residue was purified by prep-HPLC (column: 52-Welch Xtimate C18 150×30 mm, 5 μm; mobile phase: [H₂O (10 mM NH₄HCO₃)-ACN]; gradient: 27%-47% ACN over 11.0 min) to yield 7-(4-(3-fluoro-5-(methylamino) phenoxy)pyridin-2-yl)-3-(2-hydroxyethyl)-1-methyl-6,7-dihydroimidazo[1,5-a]pyrazin-8(5H)-one (30 mg, 72.32 μmol, 29.75% yield) as a white solid. LCMS [M+H]⁺=412.2; ¹H NMR (400 MHz, DMSO-d₆) δ=8.32 (d, J=5.6 Hz, 1H), 7.50 (d, J=2.4 Hz, 1H), 6.81 (dd, J=2.4, 5.6 Hz, 1H), 6.30 (q, J=4.0 Hz, 1H), 6.22 (td, J=2.0, 12.0 Hz, 1H), 6.17-6.10 (m, 2H), 4.81 (t, J=5.6 Hz, 1H), 4.35-4.27 (m, 2H), 4.24-4.15 (m, 2H), 3.75-3.63 (m, 2H), 2.80 (t, J=6.4 Hz, 2H), 2.66 (d, J=4.8 Hz, 3H), 2.35 (s, 3H).

### Example 71. Synthesis of 7-(4-(3,4-difluoro-5-(methylamino) phenoxy)pyridin-2-yl)-3-(2-hydroxyethyl)-1-methyl-6,7-dihydroimidazo[1,5-a]pyrazin-8(5H)-one (Compound 367)

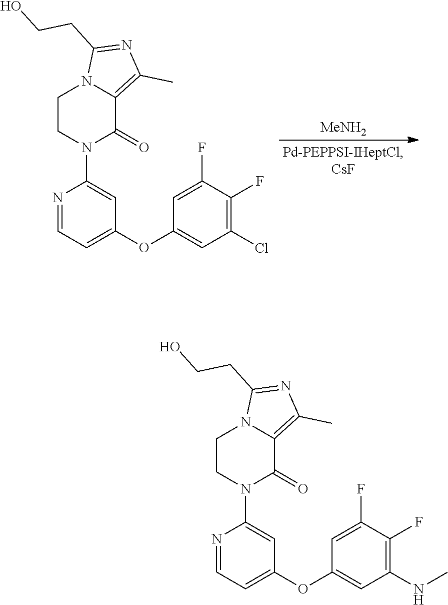 [SMILES: "MeNH2" ; Cc1nc(CCO)n2c1C(=O)N(c1cc(Oc3cc(F)c(F)c(Cl)c3)ccn1)CC2 ; CNc1cc(Oc2ccnc(N3CCn4c(CCO)nc(C)c4C3=O)c2)cc(F)c1F]

Step A: To a solution of 7-(4-(3-chloro-4,5-difluorophenoxy)pyridin-2-yl)-3-(2-hydroxyethyl)-1-methyl-6,7-dihydroimidazo[1,5-a]pyrazin-8(5H)-one (155 mg, 356.47 μmol, 1 eq.) and MeNH₂ (168.47 mg, 2.50 mmol, 7 eq, HCl salt) in DME (8 mL) was added CsF (433.19 mg, 2.85 mmol, 8 eq.), and Pd-PEPPSI-IHEPT-CI (CAS #1814936-54-3, 34.68 mg, 35.65 μmol, 0.1 eq.) under a N₂ atmosphere. The resulting mixture was stirred at 90° C. for 12 h under N₂ atmosphere. Then, the reaction was cooled to room temperature, poured into water (20 mL), and extracted with DCM (20 mL×3). The organic phase was washed with brine (50 mL), dried over anhydrous Na₂SO₄, filtered, and concentrated under vacuum. The crude product was then purified by prep-HPLC [Column: Boston Prime C18 150×30 mm, 5 μm, Condition: 29%-54% (A: H₂O (0.05% NH₃H₂O+10 mM NH₄HCO₃), B: CH₃CN), flow rate: 25 mL/min], which was further separated by SFC [ChiralPak IH, 250*30 mm, 10 μm; mobile phase: [CO₂-EtOH (0.1% NH₃H₂O)]; EtOH %: 25%] to give 7-(4-(3,4-difluoro-5-(methylamino) phenoxy)pyridin-2-yl)-3-(2-hydroxyethyl)-1-methyl-6,7-dihydroimidazo[1,5-a]pyrazin-8(5H)-one (25.77 mg, 60.01 μmol, 16.84% yield) as a white solid. LCMS [M+H]⁺=430.3; ¹H NMR (400 MHz, DMSO-d₆) δ ppm 8.31 (d, J=5.6 Hz, 1H), 7.50 (d, J=2.4 Hz, 1H), 6.80 (dd, J=2.4, 5.6 Hz, 1H), 6.46-6.38 (m, 1H), 6.34-6.22 (m, 2H), 4.84-4.76 (m, 1H), 4.35-4.26 (m, 2H), 4.26-4.17 (m, 2H), 3.75-3.64 (m, 2H), 2.80 (t, J=6.8 Hz, 2H), 2.71 (d, J=4.8 Hz, 3H), 2.35 (s, 3H).

### Example 72. Additional Compounds

The following compounds are prepared according to the procedures set forth in the above schemes and examples with modification of reagents as understood in the art.

| Compound | Structure and | LCMS | |
|---|---|---|---|
| # | IUPAC name | [M + H]⁺ | NMR |
| 85 | 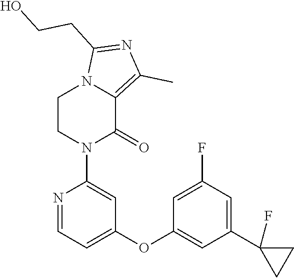 [SMILES: Cc1nc(CCO)n2c1C(=O)N(c1cc(Oc3cc(F)cc(C4(F)CC4)c3)ccn1)CC2] | 441.1 | ¹H NMR (400 MHz, DMSO-d₆) δ = 8.36 (d, J = 6.0 Hz, 1H), 7.53 (d, J = 2.0 Hz, 1H), 7.12- 7.00 (m, 2H), 6.91 (s, 1H), 6.86 (dd, J = 2.4, 6.0 Hz, 1H), 4.81 (t, J = 5.2 Hz, 1H), 4.36-4.29 (m, 2H), 4.25-4.18 (m, 2H), 3.70 (q, J = 6.8 Hz, 2H), 2.80 (t, J = 6.4 Hz, 2H), 2.35 (s, 3H), 1.58-1.47 (m, 2H), 1.36-1.25 (m, 2H). |
| | 7-(4-(3-fluoro-5-(1- | | |
| | fluorocyclopropyl)phenoxy) | | |
| | pyridin-2-yl)-3-(2-hydroxyethyl)-1- | | |
| | methyl-6,7-dihydroimidazo[1,5- | | |
| | α]pyrazin-8(5H)-one | | |
| 86 | 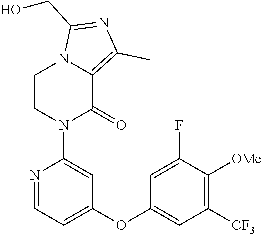 [SMILES: COc1c(F)cc(Oc2ccnc(N3CCn4c(CO)nc(C)c4C3=O)c2)cc1C(F)(F)F] | 467.0 | ¹H NMR (400 MHz, DMSO-d₆) δ = 8.36 (d, J = 5.6 Hz, 1H), 7.71 (dd, J = 2.4, 12.0 Hz, 1H), 7.57 (d, J = 2.0 Hz, 1H), 7.38 (br s, 1H), 6.96-6.78 (m, 1H), 5.48 (br s, 1H), 4.51 (br s, 2H), 4.32 (br dd, J = 6.0, 16.0 Hz, 4H), 3.96 (s, 3H), 2.36 (s, 3H). |
| | 7-(4-(3-fluoro-4-methoxy-5- | | |
| | (trifluoromethyl)phenoxy)pyridin- | | |
| | 2-yl)-3-(hydroxymethyl)-1- | | |
| | methyl-6,7-dihydroimidazo[1,5- | | |
| | α]pyrazin-8(5H)-one | | |
| 87 | 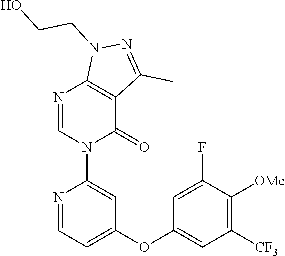 [SMILES: COc1c(F)cc(Oc2ccnc(-n3cnc4c(c(C)nn4CCO)c3=O)c2)cc1C(F)(F)F] | 480.1 | ¹H NMR (400 MHz, DMSO-d₆) δ = 8.54 (d, J = 6.0 Hz, 1H), 8.44 (s, 1H), 7.75 (dd, J = 2.8, 12.0 Hz, 1H), 7.47-7.37 (m, 2H), 7.20 (dd, J = 2.4, 6.0 Hz, 1H), 4.89 (t, J = 5.6 Hz, 1H), 4.27 (t, J = 5.8 Hz, 2H), 3.96 (d, J = 1.6 Hz, 3H), 3.83-3.75 (m, 2H), 2.45 (s, 3H). |
| | 5-(4-(3-fluoro-4-methoxy-5- | | |
| | (trifluoromethyl)phenoxy)pyridin- | | |
| | 2-yl)-1-(2-hydroxyethyl)-3- | | |
| | methyl-1,5-dihydro-4H- | | |
| | pyrazolo[3,4-d]pyrimidin-4-one | | |
| 91 | 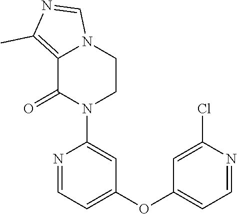 [SMILES: Cc1ncn2c1C(=O)N(c1cc(Oc3ccnc(Cl)c3)ccn1)CC2] | 356.2 | ¹H NMR (400 MHz, DMSO-d₆) δ = 8.48 (d, J = 5.6 Hz, 1H), 8.42 (d, J = 5.6 Hz, 1H), 7.81 (s, 1H), 7.73 (d, J = 2.0 Hz, 1H), 7.34 (d, J = 2.0 Hz, 1H), 7.20 (dd, J = 2.0, 5.6 Hz, 1H), 7.08 (dd, J = 2.0, 5.6 Hz, 1H), 4.41- 4.37 (m, 2H), 4.35-4.31 (m, 2H), 2.38 (s, 3H). |
| | 7-(4-((2-chloropyridin-4- | | |
| | yl)oxy)pyridin-2-yl)-1-methyl- | | |
| | 6,7-dihydroimidazo[1,5- | | |
| | α]pyrazin-8(5H)-one | | |
| 92 | 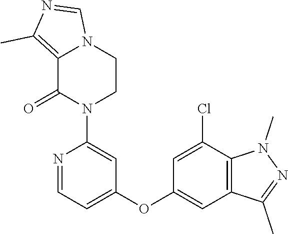 [SMILES: Cc1ncn2c1C(=O)N(c1cc(Oc3cc(Cl)c4c(c3)c(C)nn4C)ccn1)CC2] | 389.1 | ¹H NMR (400 MHz, DMSO-d₆) δ = 8.29 (d, J = 6.0 Hz, 1H), 7.84-7.74 (m, 1H), 7.68 (d, J = 9.2 Hz, 1H), 7.56 (d, J = 2.0 Hz, 1H), 7.41 (d, J = 2.4 Hz, 1H), 7.22 (dd, J = 2.4, 9.2 Hz, 1H), 6.77 (dd, J = 2.4, 6.0 Hz, 1H), 4.34-4.25 (m, 4H), 3.99 (s, 3 H), 2.45 (s, 3H), 2.33 (s, 3H). |
| | 7-(4-((1,3-dimethyl-1H-indazol- | | |
| | 5-yl)oxy)pyridin-2-yl)-1-methyl- | | |
| | 6,7-dihydroimidazo[1,5- | | |
| | α]pyrazin-8(5H)-one | | |
| 94 | 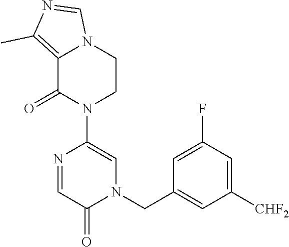 [SMILES: Cc1ncn2c1C(=O)N(c1cn(Cc3cc(F)cc(C(F)F)c3)c(=O)cn1)CC2] | 404.1 | ¹H NMR (400 MHz, DMSO-d₆) δ = 8.27 (s, 1H), 8.05 (s, 1H), 7.77 (s, 1H), 7.48 (s, 1H), 7.42 (t, J = 8.4 Hz, 2H), 7.20-6.90 (m, 1H), 5.22 (s, 2H), 4.35-4.29 (m, 2H), 4.10-4.06 (m, 2H), 2.38 (s, 3H). ¹⁹F NMR (400 MHz, DMSO- d₆) δ = −110.526, −111.320. |
| | 7-(4-(3-(difluoromethyl)-5- | | |
| | fluorobenzyl)-5-oxo-4,5- | | |
| | dihydropyrazin-2-yl)-1-methyl- | | |
| | 6,7-dihydroimidazo[1,5- | | |
| | α]pyrazin-8(5H)-one | | |
| 96 | 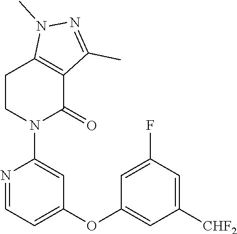 [SMILES: Cc1nn(C)c2c1C(=O)N(c1cc(Oc3cc(F)cc(C(F)F)c3)ccn1)CC2] | 401.2 | ¹H NMR (400 MHz, DMSO-d₆) δ = 8.32 (d, J = 5.2 Hz, 1H), 7.66 (s, 1H), 7.39-7.26 (m, 3H), 7.09-7.00 (m, 2H), 4.21 (t, J = 6.4 Hz, 2H), 4.07 (s, 2H), 3.71 (s, 3H), 3.01 (t, J = 6.4 Hz, 2H), 2.32 (s, 3H). ¹⁹F NMR (376 MHz, DMSO- d₆) δ = δ110.254, −111.847. |
| | 5-(4-(3-(difluoromethyl)-5- | | |
| | fluorobenzyl)pyridin-2-yl)-1,3- | | |
| | dimethyl-1,5,6,7-tetrahydro-4H- | | |
| | pyrazolo[4,3-c]pyridin-4-one | | |
| 97 | 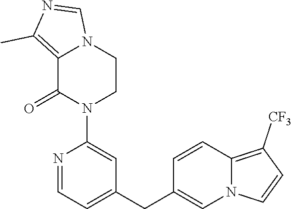 [SMILES: Cc1ncn2c1C(=O)N(c1cc(Cc3ccc4c(C(F)(F)F)ccn4c3)ccn1)CC2] | 427.3 | ¹H NMR (400 MHz, DMSO-d₆) δ = 8.96 (s, 1H), 8.44 (s, 1H), 8.35 (d, J = 5.2 Hz, 1H), 7.93- 7.68 (m, 3H), 7.43 (dd, J = 1.2, 9.2 Hz, 1H), 7.20-7.12 (m, 1H), 4.29 (s, 4H), 4.10 (s, 2H), 2.38 (br s, 3H). ¹⁹F NMR (376 MHz, DMSO- d₆) δ = −53.829. |
| | 1-methyl-7-(4-((3- | | |
| | (trifluoromethyl)pyrazolo[1,5- | | |
| | a]pyridin-6-yl)methyl)pyridin-2- | | |
| | yl)-6,7-dihydroimidazo[1,5- | | |
| | α]pyrazin-8(5H)-one | | |
| 88 | 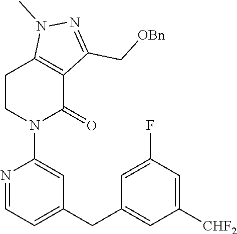 [SMILES: Cn1nc(COCc2ccccc2)c2c1CCN(c1cc(Cc3cc(F)cc(C(F)F)c3)ccn1)C2=O] | 507.5 | ¹H NMR (400 MHz, DMSO-d₆) δ = 8.33 (d, J = 5.2 Hz, 1H), 7.66 (s, 1H), 7.38-7.25 (m, 8H), 7.15-6.87 (m, 2H), 4.67 (s, 2H), 4.55 (s, 2H), 4.23 (t, J = 6.8 Hz, 2H), 4.08 (s, 2H), 3.78 (s, 3H), 3.06 (t, J = 6.8 Hz, 2H). ¹⁹F NMR (376 MHz, DMSO- d₆) δ = −110.246, −111.833. |
| | 3-((benzyloxy)methyl)-5-(4-(3- | | |
| | (difluoromethyl)-5- | | |
| | fluorobenzyl)pyridin-2-yl)-1- | | |
| | methyl-1,5,6,7-tetrahydro-4H- | | |
| | pyrazolo[4,3-c]pyridin-4-one | | |
| 89 | 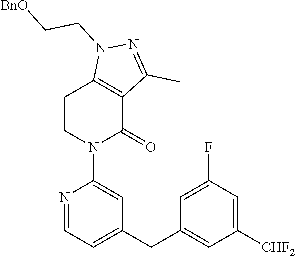 [SMILES: Cc1nn(CCOCc2ccccc2)c2c1C(=O)N(c1cc(Cc3cc(F)cc(C(F)F)c3)ccn1)CC2] | 521.2 | ¹H NMR (400 MHz, DMSO-d₆) δ = 8.31 (d, J = 5.2 Hz, 1H), 7.67 (s, 1H), 7.39-7.18 (m, 8H), 7.16-6.87 (m, 2H), 4.44 (s, 2H), 4.22 (t, J = 5.2 Hz, 2H), 4.16 (br t, J = 6.4 Hz, 2H), 4.07 (s, 2H), 3.74 (t, J = 5.2 Hz, 2H), 3.00 (t, J = 6.4 Hz, 2H), 2.34 (s, 3H). ¹⁹F NMR (376 MHz, DMSO- d₆) δ = −110.248, −111.847. |
| | 1-(2-(benzyloxy)ethyl)-5-(4-(3- | | |
| | (difluoromethyl)-5- | | |
| | fluorobenzyl)pyridin-2-yl)-3- | | |
| | methyl-1,5,6,7-tetrahydro-4H- | | |
| | pyrazolo[4,3-c]pyridin-4-one | | |
| 90 | 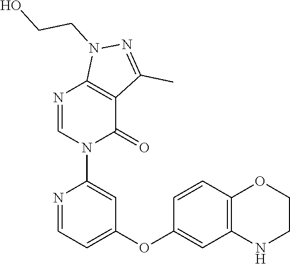 [SMILES: Cc1nn(CCO)c2ncn(-c3cc(Oc4ccc5c(c4)NCCO5)ccn3)c(=O)c12] | 421.0 | ¹H NMR (400 MHz, CDCl₃) δ = 8.38-8.36 (m, 2H), 7.25 (s, 1H), 6.91 (dd, J = 2.0, 5.6 Hz, 1H), 6.79 (d, J = 7.6 Hz, 1H), 6.41-6.35 (m, 2H), 4.48-4.41 (m, 2H), 4.29-4.23 (m, 2H), 4.10-4.01 (m, 2H), 3.98-3.84 (m, 1H), 3.48-3.41 (m, 2H), 3.27 (br t, J = 5.6 Hz, 1H), 2.59 (s, 3H). |
| | 5-(4-((3,4-dihydro-2H- | | |
| | benzo[b][1,4]oxazin-6- | | |
| | yl)oxy)pyridin-2-yl)-1-(2- | | |
| | hydroxyethyl)-3-methyl-1,5- | | |
| | dihydro-4H-pyrazolo[3,4- | | |
| | d]pyrimidin-4-one | | |
| 100 | 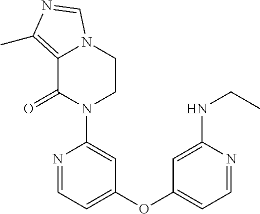 [SMILES: CCNc1cc(Oc2ccnc(N3CCn4cnc(C)c4C3=O)c2)ccn1] | 365.3 | ¹H NMR (400 MHz, DMSO-d₆) δ = 8.40 (d, J = 5.6 Hz, 1H), 8.16 (s, 1H), 8.00 (d, J = 5.6 Hz, 1H), 7.80 (s, 1H), 7.61 (d, J = 2.4 Hz, 1H), 6.94 (dd, J = 2.4, 5.6 Hz, 1H), 6.67 (t, J = 5.2 Hz, 1H), 6.27 (dd, J = 2.0, 5.6 Hz, 1H), 6.12 (d, J = 2.0 Hz, 1H), 4.45-4.31 (m, 4H), 3.25 (dd, J = 5.6, 7.2 Hz, 2H), 2.39 (s, 3H), 1.12 (t, J = 7.2 Hz, 3H). |
| | 7-(4-((2-(ethylamino)pyridin-4- | | |
| | yl)oxy)pyridin-2-yl)-1-methyl- | | |
| | 6,7-dihydroimidazo[1,5- | | |
| | α]pyrazin-8(5H)-one | | |
| 102 | 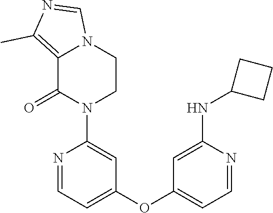 [SMILES: Cc1ncn2c1C(=O)N(c1cc(Oc3ccnc(NC4CCC4)c3)ccn1)CC2] | 391.4 | ¹H NMR δ = 8.39 (d, J = 5.6 Hz, 1H), 7.97 (d, J = 5.6 Hz, 1H), 7.79 (s, 1H), 7.60 (d, J = 2.4 Hz, 1H), 7.02-6.89 (m, 2H), 6.27 (dd, J = 2.0, 5.6 Hz, 1H), 6.06 (d, J = 2.0 Hz, 1H), 4.40-4.20 (m, 5H), 2.38 (s, 3H), 2.30-2.18 (m, 2H), 1.89-1.76 (m, 2H), 1.71-1.55 (m, 2H). |
| | 7-(4-((2- | | |
| | (cyclobutylamino)pyridin-4- | | |
| | yl)oxy)pyridin-2-yl)-1-methyl- | | |
| | 6,7-dihydroimidazo[1,5- | | |
| | α]pyrazin-8(5H)-one | | |
| 103 | 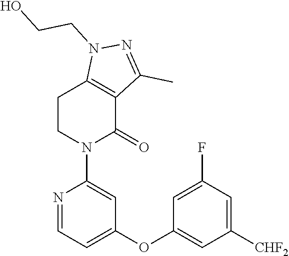 [SMILES: Cc1nn(CCO)c2c1C(=O)N(c1cc(Oc3cc(F)cc(C(F)F)c3)ccn1)CC2] | 431.1 | ¹H NMR (400 MHz, DMSO-d₆) δ = 8.32 (d, J = 5.2 Hz, 1H), 7.66 (s, 1H), 7.36 (br s, 2H), 7.29 (br d, J = 9.6 Hz, 1H), 7.18-6.85 (m, 2H), 4.90 (t, J = 5.6 Hz, 1H), 4.20 (t, J = 6.8 Hz, 2H), 4.09-4.01 (m, 4H), 3.69 (s, 2H), 3.03 (t, J = 6.4 Hz, 2H), 2.33 (s, 3H). ¹⁹F NMR (376 MHz, DMSO- d₆) δ = −110.248, −111.841. |
| | 5-(4-(3-(difluoromethyl)-5- | | |
| | fluorobenzyl)pyridin-2-yl)-1-(2- | | |
| | hydroxyethyl)-3-methyl-1,5,6,7- | | |
| | tetrahydro-4H-pyrazolo[4,3- | | |
| | c]pyridin-4-one | | |
| 104 | 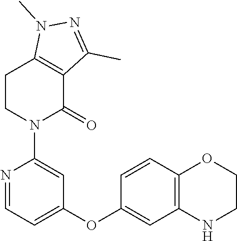 [SMILES: Cc1nn(C)c2c1C(=O)N(c1cc(Oc3ccc4c(c3)NCCO4)ccn1)CC2] | 392.2 | ¹H NMR (400 MHz, DMSO-d₆) δ = 8.25 (d, J = 5.6 Hz, 1H), 7.22 (d, J = 2.4 Hz, 1H), 6.74- 6.63 (m, 2H), 6.32 (d, J = 2.8 Hz, 1H), 6.22 (dd, J = 2.8, 8.4 Hz, 1H), 6.08-6.02 (m, 1H), 4.22 (t, J = 6.4 Hz, 2H), 4.11 (t, J = 4.0 Hz, 2H), 3.70 (s, 3H), 3.31-3.24 (m, 2H), 3.00 (t, J = 6.4 Hz, 2H), 2.29 (s, 3H). |
| | 5-(4-((3,4-dihydro-2H- | | |
| | benzo[b][1,4]oxazin-6- | | |
| | yl)oxy)pyridin-2-yl)-1,3- | | |
| | dimethyl-1,5,6,7-tetrahydro-4H- | | |
| | pyrazolo[4,3-c]pyridin-4-one | | |
| 105 | 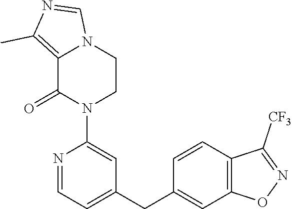 [SMILES: Cc1ncn2c1C(=O)N(c1cc(Cc3ccc4c(C(F)(F)F)noc4c3)ccn1)CC2] | 428.1 | ¹H NMR (400 MHz, DMSO-d₆) δ = 8.34 (d, J = 5.2 Hz, 1H), 8.00 (s, 1H), 7.93 (d, J = 8.4 Hz, 1H), 7.85 (s, 1H), 7.78 (s, 1H), 7.57-7.49 (m, 1H), 7.18-7.10 (m, 1H), 4.33-4.23 (m, 6H), 2.39 (s, 3H). ¹⁹F NMR (376 MHz, DMSO- d₆) δ = −61.669 |
| | 1-methyl-7-(4-((3- | | |
| | (trifluoromethyl)benzo[d]isoxazo | | |
| | 1-6-yl)methyl)pyridin-2-yl)-6,7- | | |
| | dihydroimidazo[1,5-α]pyrazin- | | |
| | 8(5H)-one | | |
| 106 | 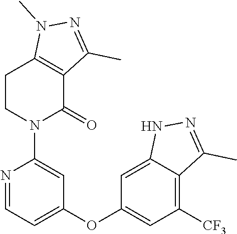 [SMILES: Cc1nn(C)c2c1C(=O)N(c1cc(Oc3cc(C(F)(F)F)c4c(C)n[nH]c4c3)ccn1)CC2] | 407.2 | ¹H NMR (400 MHz, DMSO-d₆) δ = 12.98 (br s, 1H), 8.32 (d, J = 5.6 Hz, 1H), 7.36 (d, J = 2.0 Hz, 1H), 7.05 (d, J = 1.6 Hz, 1H), 6.83 (dd, J = 2.4, 5.6 Hz, 1H), 6.79-6.71 (m, 1H), 4.26 (t, J = 6.4 Hz, 2H), 3.70 (s, 3H), 3.05-2.96 (m, 2H), 2.56 (s, 3H) 2.27 (s, 3H). ¹⁹F NMR (376 MHz, CDCl₃) δ = −118.019. |
| | 5-(4-((4-fluoro-3-methyl-1H- | | |
| | indazol-6-yl)oxy)pyridin-2-yl)- | | |
| | 1,3-dimethyl-1,5,6,7-tetrahydro- | | |
| | 4H-pyrazolo[4,3-c]pyridin-4-one | | |
| 108 | 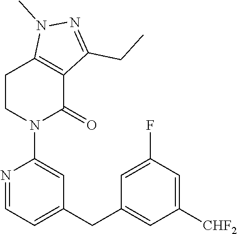 [SMILES: CCc1nn(C)c2c1C(=O)N(c1cc(Cc3cc(F)cc(C(F)F)c3)ccn1)CC2] | 415.3 | ¹H NMR (400 MHz, DMSO-d₆ 8 = 8.32 (d, J = 5.2 Hz, 1H) 7.65 (s, 1H), 7.36 (s, 2H), 7.29 (d, J = 8.8 Hz, 1H), 7.18-6.8: (m, 2H), 4.20 (t, J = 6.8 Hz 2H), 4.07 (s, 2H), 3.72 (s, 3H) 3.01 (t, J = 6.4 Hz, 2H), 2.76 (q J = 7.6 Hz, 2H), 1.17 (t, J = 7.6 Hz, 3H). ¹⁹F NMR (376 MHz, DMSO d₆) δ = −110.240, −111.851. |
| | 5-(4-(3-(difluoromethyl)-5- | | |
| | fluorobenzyl)pyridin-2-yl)-3- | | |
| | ethyl-1-methyl-1,5,6,7- | | |
| | tetrahydro-4H-pyrazolo[4,3- | | |
| | c]pyridin-4-one | | |
| 110 | 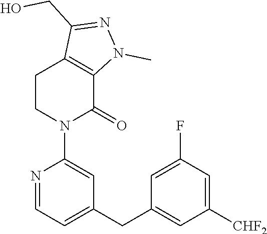 [SMILES: Cn1nc(CO)c2c1C(=O)N(c1cc(Cc3cc(F)cc(C(F)F)c3)ccn1)CC2] | 417.3 | ¹H NMR (400 MHz, DMSO-d₆ δ = 8.37 (d, J = 5.2 Hz, 1H) 7.70 (s, 1H), 7.37 (br s, 2H) 7.30 (br d, J = 9.2 Hz, 1H) 7.17-6.88 (m, 2H), 5.13-5.0- (m, 1H), 4.45 (d, J = 5.2 Hz 2H), 4.19 (t, J = 6.4 Hz, 2H) 4.10 (s, 2H), 4.04 (s, 3H), 2.81 (t, J = 6.4 Hz, 2H). ¹⁹F NMR (376 MHz, DMSO d₆) δ = −110.264, −111.791. |
| | 6-(4-(3-(difluoromethyl)-5- | | |
| | fluorobenzyl)pyridin-2-yl)-3- | | |
| | (hydroxymethyl)-1-methyl- | | |
| | 1,4,5,6-tetrahydro-7H- | | |
| | pyrazolo[3,4-c]pyridin-7-one | | |
| 112 | 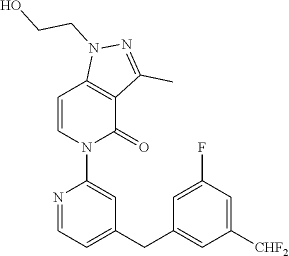 [SMILES: Cc1nn(CCO)c2ccn(-c3cc(Cc4cc(F)cc(C(F)F)c4)ccn3)c(=O)c12] | 429.2 | ¹H NMR (400 MHz, DMSO-d₆ δ = 8.49 (d, J = 5.2 Hz, 1H) 7.73 (s, 1H), 7.67 (d, J = 7.6 Hz 1H), 7.43-7.38 (m, 2H), 7.38 7.28 (m, 2H), 7.16 (t, J = 55.( Hz, 1H), 6.71 (d, J = 7.6 Hz 1H), 4.88 (t, J = 5.4 Hz, 1H) 4.24 (t, J = 5.2 Hz, 2H), 4.16 (s 2H), 3.81-3.69 (m, 2H), 2.48 (s 3H). ¹⁹F NMR (400 MHz, DMSO d₆) δ = −110.290, −111.698. |
| | 5-(4-(3-(difluoromethyl)-5- | | |
| | fluorobenzyl)pyridin-2-yl)-1-(2- | | |
| | hydroxyethyl)-3-methyl-1,5- | | |
| | dihydro-4H-pyrazolo[4,3- | | |
| | c]pyridin-4-one | | |
| 113 | 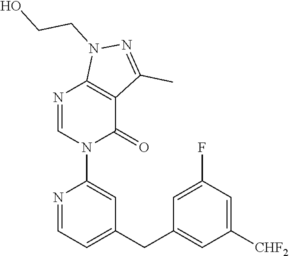 [SMILES: Cc1nn(CCO)c2ncn(-c3cc(Cc4cc(F)cc(C(F)F)c4)ccn3)c(=O)c12] | 430.1 | ¹H NMR (400 MHz, CDCl₃) δ = 8.52 (d, J = 4.8 Hz, 1H), 8.47 (s, 1H), 7.67 (s, 1H), 7.18-7.13 (m, 3H), 7.04 (br d, J = 8.8 Hz, 1H), 6.61 (t, J = 56.0 Hz, 1H), 4.48-4.45 (m, 2H), 4.12-4.07 (m, 4H), 3.25 (br t, J = 5.6 Hz, 1H), 2.62 (s, 3H). ¹⁹F NMR (400 MHz, CDCl₃) δ = −110.630, −111.613. |
| | 5-(4-(3-(difluoromethyl)-5- | | |
| | fluorobenzyl)pyridin-2-yl)-1-(2- | | |
| | hydroxyethyl)-3-methyl-1,5- | | |
| | dihydro-4H-pyrazolo[3,4- | | |
| | d]pyrimidin-4-one | | |
| 114 | 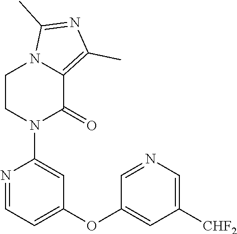 [SMILES: Cc1nc(C)n2c1C(=O)N(c1cc(Oc3cncc(C(F)F)c3)ccn1)CC2] | 386.2 | ¹H NMR (400 MHz, DMSO-d₆) δ = 8.74 (d, J = 6.0 Hz, 2H), 8.40 (d, J = 5.6 Hz, 1H), 7.93 (s, 1H), 7.59 (d, J = 2.0 Hz, 1H), 7.36-7.03 (m, 1H), 6.91 (dd, J = 2.4, 5.6 Hz, 1H), 4.38-4.30 (m, 2H), 4.22-4.13 (m, 2H), 2.32 (d, J = 8.8 Hz, 6H). ¹⁹F NMR (376 MHz, DMSO- d₆) δ = −111.553. |
| | 7-(4-((5-(difluoromethyl)pyridin- | | |
| | 3-yl)oxy)pyridin-2-yl)-1,3- | | |
| | dimethyl-6,7- | | |
| | dihydroimidazo[1,5-α]pyrazin- | | |
| | 8(5H)-one | | |
| 115 | 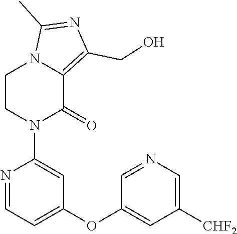 [SMILES: Cc1nc(CO)c2n1CCN(c1cc(Oc3cncc(C(F)F)c3)ccn1)C2=O] | 402.2 | ¹H NMR (400 MHz, DMSO- d₆) δ = 8.73 (d, J = 4.8 Hz, 2H), 8.41 (d, J = 6.0 Hz, 1H), 7.93 (s, 1H), 7.56 (d, J = 2.0 Hz, 1H), 7.19 (t, J = 54.8 Hz, 1H), 6.93 (dd, J = 5.6, 5.6 Hz, 1H), 4.92 (t, J = 6.0 Hz, 1H), 4.55 (d, J = 5.6 Hz, 2H), 4.38-4.35 (m, 2H), 4.23-4.20 (m, 2H), 2.35 (s, 3H). ¹⁹F NMR (400 MHz, DMSO- d₆) δ = −111.554. |
| | 7-(4-((5-(difluoromethyl)pyridin- | | |
| | 3-yl)oxy)pyridin-2-yl)-1- | | |
| | (hydroxymethyl)-3-methyl-6,7- | | |
| | dihydroimidazo[1,5-α]pyrazin- | | |
| | 8(5H)-one | | |
| 116 | 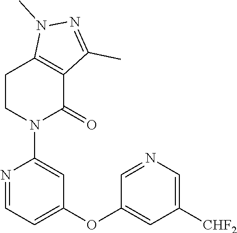 [SMILES: Cc1nn(C)c2c1C(=O)N(c1cc(Oc3cncc(C(F)F)c3)ccn1)CC2] | 386.2 | ¹H NMR (400 MHz, DMSO-d₆) δ = 8.72-8.70 (m, 2H), 8.37 (d, J = 5.6 Hz, 1H), 7.89 (s, 1H), 7.43 (d, J = 2.0 Hz, 1H), 7.18 (t, J = 55.2 Hz, 1H), 6.86 (dd, J = 2.4, 6.0 Hz, 1H), 4.28 (t, J = 6.8 Hz, 2H), 3.70 (s, 3H), 3.02 (t, J = 6.4 Hz, 2H), 2.29 (s, 3H). ¹⁹F NMR (400 MHz, DMSO- d₆) δ = −111.544. |
| | 5-(4-((5-(difluoromethyl)pyridin- | | |
| | 3-yl)oxy)pyridin-2-yl)-1,3- | | |
| | dimethyl-1,5,6,7-tetrahydro-4HI- | | |
| | pyrazolo[4,3-c]pyridin-4-one | | |
| 117 | 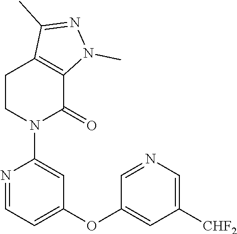 [SMILES: Cc1nn(C)c2c1CCN(c1cc(Oc3cncc(C(F)F)c3)ccn1)C2=O] | 386.2 | ¹H NMR (400 MHz, DMSO-d₆) δ = 8.76-8.69 (m, 2H), 8.41 (d, J = 5.6 Hz, 1H), 7.92 (s, 1H), 7.45 (d, J = 2.0 Hz, 1H), 7.19 (t, J = 55.2 Hz, 1H), 6.92 (dd, J = 2.4, 5.6 Hz, 1H), 4.25 (t, J = 6.4 Hz, 2H), 3.98 (s, 3H), 2.78 (t, J = 6.4 Hz, 2H), 2.15 (s, 3H). ¹⁹F NMR (400 MHz, DMSO- d₆) δ = −111.543. |
| | 6-(4-((5-(difluoromethyl)pyridin- | | |
| | 3-yl)oxy)pyridin-2-yl)-1,3- | | |
| | dimethyl-1,4,5,6-tetrahydro-7H- | | |
| | pyrazolo[3,4-c]pyridin-7-one | | |
| 118 | 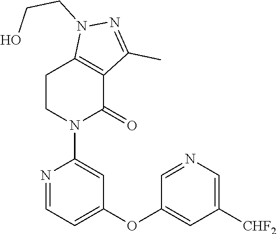 [SMILES: Cc1nn(CCO)c2c1C(=O)N(c1cc(Oc3cncc(C(F)F)c3)ccn1)CC2] | 416.2 | ¹H NMR (400 MHz, DMSO-d₆) δ = 8.72-8.71 (m, 2H), 8.37 (d, J = 5.6 Hz, 1H ), 7.90 (s, 1H), 7.43 (d, J = 2.4 Hz, 1H), 7.18 (t, J = 54.8 Hz, 1H), 6.86 (dd, J = 2.4, 5.6 Hz, 1H), 4.91 (t, J = 5.6 Hz, 1H), 4.27 (t, J = 6.4 Hz, 2H), 4.04 (t, J = 5.2 Hz, 2H), 3.68 (q, J = 5.2 Hz, 2H), 3.04 (t, J = 6.4 Hz, 2H), 2.31 (s, 3H). ¹⁹F NMR (376 MHz, DMSO-d₆) δ = −111.531. |
| | 5-(4-((5-(difluoromethyl)pyridin- | | |
| | 3-yl)oxy)pyridin-2-yl)-1-(2- | | |
| | hydroxyethyl)-3-methyl-1,5,6,7- | | |
| | tetrahydro-4H-pyrazolo[4,3- | | |
| | c]pyridin-4-one | | |
| 119 | 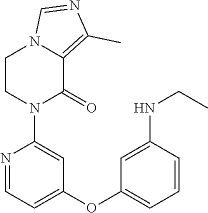 [SMILES: CCNc1cccc(Oc2ccnc(N3CCn4cnc(C)c4C3=O)c2)c1] | 365.2 | 1H NMR (400 MHz, DMSO-d6) δ = 8.33 (d, J = 5.6 Hz, 1H), 7.78 (s, 1H), 7.61 (d, J = 2.0 Hz, 1H), 7.49 (t, J = 8.0 Hz, 1H), 6.90 (dd, J = 2.0, 5.6 Hz, 1H), 6.74 (br t, J = 4.8 Hz, 1H), 6.29 (d, J = 8.4 Hz, 1H), 6.16 (d, J = 7.2 Hz, 1H), 4.39-4.31 (m, 4H), 3.15-3.08 (m, 2H), 2.38 (s, 3H), 1.05 (t, J = 7.2 Hz, 3H). |
| | 7-(4-((6-(ethylamino)pyridin-2- | | |
| | yl)oxy)pyridin-2-yl)-1-methyl- | | |
| | 6,7-dihydroimidazo[1,5- | | |
| | α]pyrazin-8(5H)-one | | |
| 121 | 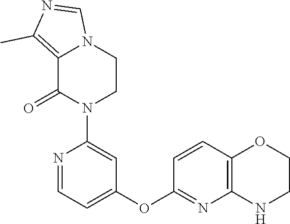 [SMILES: Cc1ncn2c1C(=O)N(c1cc(Oc3ccc4c(n3)NCCO4)ccn1)CC2] | 379.2 | ¹H NMR (400 MHz, DMSO-d₆) δ = 8.30 (d, J = 5.6 Hz, 1H), 7.78 (s, 1H), 7.47 (d, J = 2.0 Hz, 1H), 7.08 (d, J = 8.0 Hz, 1H), 7.01 (br s, 1H), 6.82-6.78 (m, 1H), 6.20 (d, J = 8.0 Hz, 1H), 4.36-4.27 (m, 4H), 4.09 (t, J = 4.4 Hz, 2H), 3.40-3.35 (m, 2H), 2.38 (s, 3H). |
| | 7-(4-((3,4-dihydro-2H- | | |
| | pyrido[3,2-b][1,4]oxazin-6- | | |
| | yl)oxy)pyridin-2-yl)-1-methyl- | | |
| | 6,7-dihydroimidazo[1,5- | | |
| | α]pyrazin-8(5H)-one | | |
| 122 | 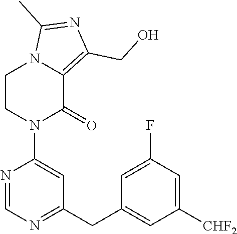 [SMILES: Cc1nc(CO)c2n1CCN(c1cc(Cc3cc(F)cc(C(F)F)c3)ncn1)C2=O] | 418.2 | ¹H NMR (400 MHz, DMSO-d₆) δ = 8.93 (d, J = 1.2 Hz, 1H), 8.11 (d, J = 0.8 Hz, 1H), 7.41- 7.36 (m, 2H), 7.30 (d, J = 8.8 Hz, 1H), 7.02 (t, J = 56.0 Hz, 1H), 4.91 (t, J = 6.0 Hz, 1H), 4.61 (d, J = 6.0 Hz, 2H), 4.44- 4.39 (m, 2H), 4.23-4.18 (m, 4H), 2.35 (s, 3H). 19F NMR (376 MHz, DMSO- d6) δ = −110.266, −112.032. |
| | 7-(6-(3-(difluoromethyl)-5- | | |
| | fluorobenzyl)pyrimidin-4-yl)-1- | | |
| | (hydroxymethyl)-3-methyl-6,7- | | |
| | dihydroimidazo[1,5-α]pyrazin- | | |
| | 8(5H)-one | | |
| 123 | 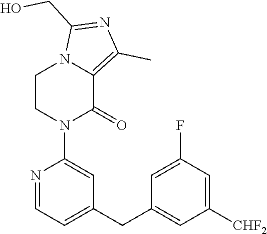 [SMILES: Cc1nc(CO)n2c1C(=O)N(c1cc(Cc3cc(F)cc(C(F)F)c3)ccn1)CC2] | 417.2 | ¹H NMR (400 MHz, DMSO-d₆) δ = 8.35 (d, J = 5.2 Hz, 1H), 7.89 (s, 1H), 7.41-7.25 (m, 3H), 7.19-6.82 (m, 2H), 5.65-5.34 (m, 1H), 4.56-4.48 (m, 2H), 4.36-4.23 (m, 4H), 4.15-4.06 (m, 2H), 2.42-2.35 (m, 3H). ¹⁹F NMR (376 MHz, DMSO- d₆) δ = −110.252, −111.821. |
| | 7-(4-(3-(difluoromethyl)-5- | | |
| | fluorobenzyl)pyridin-2-yl)-3- | | |
| | (hydroxymethyl)-1-methyl-6,7- | | |
| | dihydroimidazo[1,5-α]pyrazin- | | |
| | 8(5H)-one | | |
| 124 | 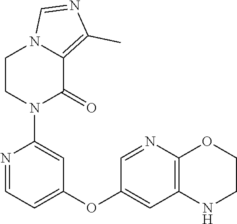 [SMILES: Cc1ncn2c1C(=O)N(c1cc(Oc3cnc4c(c3)NCCO4)ccn1)CC2] | 377.3 | ¹H NMR (400 MHz, DMSO-d₆) δ = 8.32 (d, J = 5.2 Hz, 1H), 7.78 (s, 1H), 7.75 (s, 1H), 7.31 (d, J = 2.0 Hz, 1H), 7.04 (dd, J = 1.6, 5.2 Hz, 1H), 6.70 (d, J = 2.0 Hz, 1H), 6.02 (s, 1H), 4.30 (s, 4H), 4.22-4.16 (m, 2H), 3.81 (s, 2H), 3.26-3.16 (m, 2H), 2.40 (s, 3H). |
| | 7-(4-((2,3-dihydro-1H- | | |
| | pyrido[2,3-b][1,4]oxazin-7- | | |
| | yl)methyl)pyridin-2-yl)-1- | | |
| | methyl-6,7-dihydroimidazo[1,5- | | |
| | α]pyrazin-8(5H)-one | | |
| 125 | 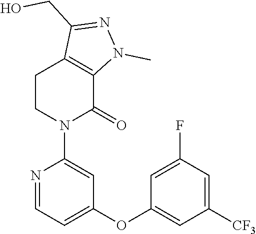 [SMILES: Cn1nc(CO)c2c1C(=O)N(c1cc(Oc3cc(F)cc(C(F)(F)F)c3)ccn1)CC2] | 437.1 | ¹H NMR (400 MHz, DMSO-d₆) δ = 8.42 (d, J = 5.6 Hz, 1H), 7.65 (br d, J = 8.4 Hz, 1H), 7.56 (br d, J = 9.6 Hz, 1H), 7.52-7.45 (m, 2H), 6.94 (dd, J = 2.4, 6.0 Hz, 1H), 5.09 (t, J = 4.8 Hz, 1H), 4.45 (d, J = 4.8 Hz, 2H), 4.25 (t, J = 6.4 Hz, 2H), 4.01 (s, 3H), 2.89 (t, J = 6.4 Hz, 2H). ¹⁹F NMR (376 MHz, DMSO- d₆) δ = −61.286, −106.959. |
| | 6-(4-(3-fluoro-5- | | |
| | (trifluoromethyl)phenoxy)pyridin- | | |
| | 2-yl)-3-(hydroxymethyl)-1- | | |
| | methyl-1,4,5,6-tetrahydro-7H- | | |
| | pyrazolo[3,4-c]pyridin-7-one | | |
| 126 | 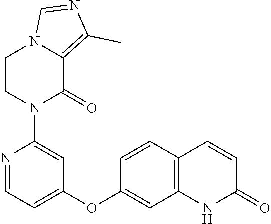 [SMILES: Cc1ncn2c1C(=O)N(c1cc(Oc3ccc4ccc(=O)[nH]c4c3)ccn1)CC2] | 388.2 | ¹H NMR (400 MHz, DMSO-d₆) δ = 11.77 (s, 1H), 8.38 (d, J = 5.6 Hz, 1H), 8.01-7.69 (m, 3H), 7.55 (d, J = 2.0 Hz, 1H), 7.06- 6.96 (m, 2H), 6.91 (dd, J = 2.4, 5.6 Hz, 1H), 6.47 (dd, J = 1.6, 9.6 Hz, 1H), 4.45-4.20 (m, 4H), 2.35 (s, 3H). |
| | 7-((2-(1-methyl-8-oxo-5,6- | | |
| | dihydroimidazo[1,5-α]pyrazin- | | |
| | 7(8H)-yl)pyridin-4- | | |
| | yl)oxy)quinolin-2(1H)-one | | |
| 127 | 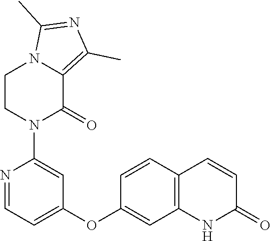 [SMILES: Cc1nc(C)n2c1C(=O)N(c1cc(Oc3ccc4ccc(=O)[nH]c4c3)ccn1)CC2] | 402.3 | ¹H NMR (400 MHz, DMSO-d₆) δ = 11.76 (s, 1H), 8.37 (d, J = 5.6 Hz, 1H), 7.93 (d, J = 9.6 Hz, 1H), 7.77 (d, J = 8.8 Hz, 1H), 7.55 (d, J = 2.4 Hz, 1H), 7.04- 6.98 (m, 2H), 6.90 (dd, J = 2.4, 6.0 Hz, 1H), 6.47 (d, J = 9.6 Hz, 1H), 4.36-4.32 (m, 2H), 4.18- 4.15 (m, 2H), 2.31 (d, J = 4.8 Hz, 6H). |
| | 7-((2-(1,3-dimethyl-8-oxo-5,6- | | |
| | dihydroimidazo[1,5-α]pyrazin- | | |
| | 7(8H)-yl)pyridin-4- | | |
| | yl)oxy)quinolin-2(1H)-one | | |
| 132 | 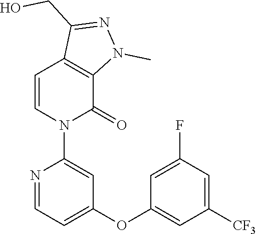 [SMILES: Cn1nc(CO)c2ccn(-c3cc(Oc4cc(F)cc(C(F)(F)F)c4)ccn3)c(=O)c21] | 435.2 | ¹H NMR (400 MHz, DMSO-d₆) δ = 8.56 (d, J = 5.6 Hz, 1H), 7.68 (d, J = 8.8 Hz, 1H), 7.61 (dd, J = 2.0, 9.6 Hz, 1H), 7.56 (s, 1H), 7.54 (d, J = 2.0 Hz, 1H), 7.46 (d, J = 7.2 Hz, 1H), 7.18 (dd, J = 2.4, 5.6 Hz, 1H), 6.77 (d, J = 7.6 Hz, 1H), 5.32 (t, J = 5.6 Hz, 1H), 4.66 (d, J = 5.6 Hz, 2H), 4.21 (s, 3H). ¹⁹F NMR (376 MHz, DMSO- d₆) δ = −61.262, −106.816. |
| | 6-(4-(3-fluoro-5- | | |
| | (trifluoromethyl)phenoxy)pyridin- | | |
| | 2-yl)-3-(hydroxymethyl)-1- | | |
| | methyl-1,6-dihydro-7H- | | |
| | pyrazolo[3,4-c]pyridin-7-one | | |
| 133 | 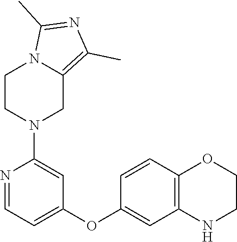 [SMILES: Cc1nc(C)n2c1CN(c1cc(Oc3ccc4c(c3)NCCO4)ccn1)CC2] | 392.3 | ¹H NMR (400 MHz, DMSO-d₆) δ = 8.27 (d, J = 5.6 Hz, 1H), 7.38 (d, J = 2.0 Hz, 1H), 6.74- 6.67 (m, 2H), 6.33 (d, J = 2.8 Hz, 1H), 6.23 (dd, J = 2.8, 11.6 Hz, 1H), 6.06 (s, 1H), 4.33-4.26 (m, 2H), 4.18-4.09 (m, 4H), 3.29 (d, J = 2.4 Hz, 2H), 2.31 (d, J = 12.8 Hz, 6H). |
| | 7-(4-((3,4-dihydro-2H- | | |
| | benzo[b][1,4]oxazin-6- | | |
| | yl)oxy)pyridin-2-yl)-1,3- | | |
| | dimethyl-6,7- | | |
| | dihydroimidazo[1,5-α]pyrazin- | | |
| | 8(5H)-one | | |
| 134 | 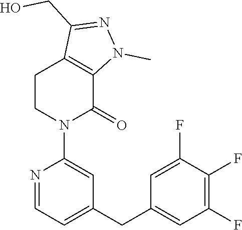 [SMILES: Cn1nc(CO)c2c1C(=O)N(c1cc(Cc3cc(F)c(F)c(F)c3)ccn1)CC2] | 403.2 | ¹H NMR (500 MHz, DMSO-d₆) δ = 8.36 (d, J = 5.0 Hz, 1H), 7.68 (s, 1H), 7.32-7.28 (m, 2H), 7.13 (dd, J = 1.5, 5.0 Hz, 1H), 5.08 (t, J = 5.5 Hz, 1H), 4.45 (d, J = 5.5 Hz, 2H), 4.18 (t, J = 6.5 Hz, 2H), 4.04 (s, 3H), 4.00 (s, 2H), 2.88 (t, J = 6.5 Hz, 2H) ¹⁹F NMR (376 MHz, DMSO- d₆) δ = −135.278, −164.594. |
| | 3-(hydroxymethyl)-1-methyl-6- | | |
| | (4-(3,4,5-trifluorobenzyl)pyridin- | | |
| | 2-yl)-1,4,5,6-tetrahydro-7H- | | |
| | pyrazolo[3,4-c]pyridin-7-one | | |
| 135 | 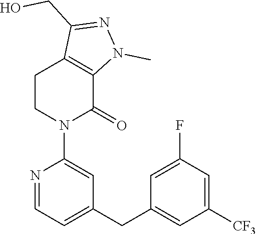 [SMILES: Cn1nc(CO)c2c1C(=O)N(c1cc(Cc3cc(F)cc(C(F)(F)F)c3)ccn1)CC2] | 435.3 | ¹H NMR (500 MHz, DMSO-d₆) δ = 8.37 (d, J = 5.5 Hz, 1H), 7.72 (s, 1H), 7.59 (s, 1H), 7.53 (t, J = 8.5 Hz, 2H), 7.15 (dd, J = 1.5, 5.5 Hz, 1H), 5.09 (t, J = 6.0 Hz, 1H), 4.45 (d, J = 5.5 Hz, 2H), 4.19 (t, J = 6.5 Hz, 2H), 4.14 (s, 2H), 4.04 (s, 3H), 2.94- 2.85 (m, 2H). ¹⁹F NMR (376 MHz, DMSO- d₆) δ = −61.065, −110.622. |
| | 6-(4-(3-fluoro-5- | | |
| | (trifluoromethyl)benzyl)pyridin- | | |
| | 2-yl)-3-(hydroxymethyl)-1- | | |
| | methyl-1,4,5,6-tetrahydro-7HI- | | |
| | pyrazolo[3,4-c]pyridin-7-one | | |
| 137 | 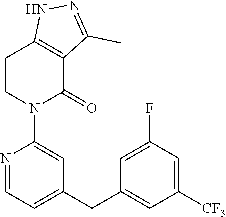 [SMILES: Cc1n[nH]c2c1C(=O)N(c1cc(Cc3cc(F)cc(C(F)(F)F)c3)ccn1)CC2] | 405.2 | ¹H NMR (400 MHz, DMSO-d₆) δ = 8.34 (d, J = 4.8 Hz, 1H), 7.71 (s, 1H), 7.59 (s, 1H), 7.56- 7.51 (m, 2H), 7.12 (d, J = 5.2 Hz, 1H), 4.18 (s, 2H), 4.13 (s, 2H), 2.92 (t, J = 6.4 Hz, 2H), 2.41 (s, 3H) ¹⁹F NMR (376 MHz, DMSO- d₆) δ = −61.059, −110.646. |
| | 5-(4-(3-fluoro-5- | | |
| | (trifluoromethyl)benzyl)pyridin- | | |
| | 2-yl)-3-methyl-1,5,6,7- | | |
| | tetrahydro-4H-pyrazolo[4,3- | | |
| | c]pyridin-4-one | | |
| 138 | 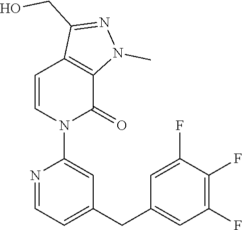 [SMILES: Cn1nc(CO)c2ccn(-c3cc(Cc4cc(F)c(F)c(F)c4)ccn3)c(=O)c21] | 401.2 | ¹H NMR (400 MHz, DMSO-d₆) δ = 8.52 (d, J = 4.8 Hz, 1H), 7.74 (s, 1H), 7.40 (d, J = 7.2 Hz, 2H), 7.38-7.31 (m, 2H), 6.76 (d, J = 7.6 Hz, 1H), 5.32 (t, J = 5.8 Hz, 1H), 4.66 (d, J = 5.6 Hz, 2H), 4.23 (s, 3H), 4.08 (s, 2H). ¹⁹F NMR (376 MHz, DMSO- d₆) δ = −135.248, −164.462. |
| | 3-(hydroxymethyl)-1-methyl-6- | | |
| | (4-(3,4,5-trifluorobenzyl)pyridin- | | |
| | 2-yl)-1,6-dihydro-7H- | | |
| | pyrazolo[3,4-c]pyridin-7-one | | |
| 139 | 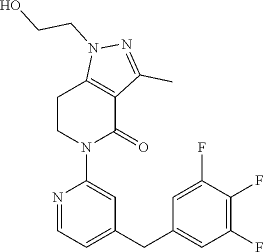 [SMILES: Cc1nn(CCO)c2c1C(=O)N(c1cc(Cc3cc(F)c(F)c(F)c3)ccn1)CC2] | 417.2 | ¹H NMR (400 MHz, DMSO-d₆) δ = 8.31 (d, J = 5.2 Hz, 1H), 7.64 (s, 1H), 7.29 (dd, J = 6.8, 8.8 Hz, 2H), 7.05 (dd, J = 1.2, 5.2 Hz, 1H), 4.91 (s, 1H), 4.19 (t, J = 6.8 Hz, 2H), 4.04 (t, J = 5.2 Hz, 2H), 3.97 (s, 2H), 3.69 (s, 2H), 3.03 (t, J = 6.4 Hz, 2H), 2.33 (s, 3H). ¹⁹F NMR (376 MHz, DMSO- d₆) δ = −135.380, −164.665. |
| | 1-(2-hydroxyethyl)-3-methyl-5- | | |
| | (4-(3,4,5-trifluorobenzyl)pyridin- | | |
| | 2-yl)-1,5,6,7-tetrahydro-4H- | | |
| | pyrazolo[4,3-c]pyridin-4-one | | |
| 140 | 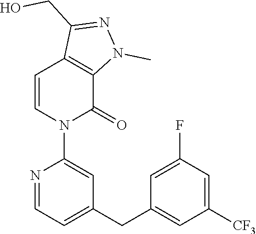 [SMILES: Cn1nc(CO)c2ccn(-c3cc(Cc4cc(F)cc(C(F)(F)F)c4)ccn3)c(=O)c21] | 433.2 | ¹H NMR (400 MHz, DMSO-d₆) δ = 8.52 (d, J = 4.8 Hz, 1H), 7.78 (s, 1H), 7.64 (s, 1H), 7.59- 7.54 (m, 2H), 7.44-7.40 (m, 2H), 6.76 (d, J = 7.6 Hz, 1H), 5.32 (t, J = 5.8 Hz, 1H), 4.66 (d, J = 5.8 Hz, 2H), 4.23 (s, 3H), 4.21 (s, 2H). ¹⁹F NMR (376 MHz, DMSO- d₆) δ = −61.065, −110.538. |
| | 6-(4-(3-fluoro-5- | | |
| | (trifluoromethyl)benzyl)pyridin- | | |
| | 2-yl)-3-(hydroxymethyl)-1- | | |
| | methyl-1,6-dihydro-7H- | | |
| | pyrazolo[3,4-c]pyridin-7-one | | |
| 141 | 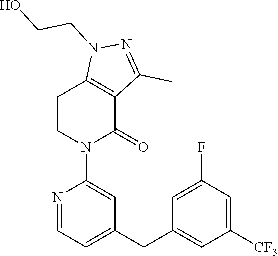 [SMILES: Cc1nn(CCO)c2c1C(=O)N(c1cc(Cc3cc(F)cc(C(F)(F)F)c3)ccn1)CC2] | 449.2 | ¹H NMR (400 MHz, DMSO-d₆) δ = 8.32 (d, J = 4.8 Hz, 1H), 7.68 (s, 1H), 7.58 (s, 1H), 7.53 (t, J = 8.8 Hz, 2H), 7.07 (dd, J = 1.2, 5.2 Hz, 1H), 4.90 (t, J = 5.2 Hz, 1H), 4.20 (t, J = 6.4 Hz, 2H), 4.11 (s, 2H), 4.04 (t, J = 5.2 Hz, 2H), 3.69 (q, J = 5.2 Hz, 2H), 3.03 (t, J = 6.8 Hz, 2H), 2.33 (s, 3H). ¹⁹F NMR (376 MHz, DMSO- d₆) δ = −61.065, −110.682. |
| | 5-(4-(3-fluoro-5- | | |
| | (trifluoromethyl)benzyl)pyridin- | | |
| | 2-yl)-1-(2-hydroxyethyl)-3- | | |
| | methyl-1,5,6,7-tetrahydro-4H- | | |
| | pyrazolo[4,3-c]pyridin-4-one | | |
| 143 | 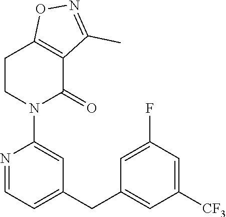 [SMILES: Cc1noc2c1C(=O)N(c1cc(Cc3cc(F)cc(C(F)(F)F)c3)ccn1)CC2] | 406.1 | ¹H NMR (400 MHz, DMSO-d₆) δ = 8.36 (d, J = 4.8 Hz, 1H), 7.71-7.57 (m, 2H), 7.53 (t, J = 8.4 Hz, 2H), 7.14 (d, J = 4.4 Hz, 1H), 4.32 (t, J = 6.4 Hz, 2H), 4.13 (s, 2H), 3.24 (t, J = 6.4 Hz, 2H), 2.41 (s, 3H). ¹⁹F NMR (376 MHz, DMSO- d₆) δ = −61.085, −110.642 |
| | 5-(4-(3-fluoro-5- | | |
| | (trifluoromethyl)benzyl)pyridin- | | |
| | 2-yl)-3-methyl-6,7- | | |
| | dihydroisoxazolo[4,5-c]pyridin- | | |
| | 4(5H)-one | | |
| 144 | 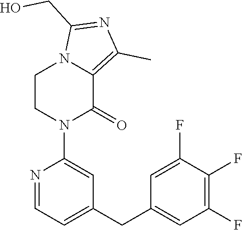 [SMILES: Cc1nc(CO)n2c1C(=O)N(c1cc(Cc3cc(F)c(F)c(F)c3)ccn1)CC2] | 403.1 | ¹H NMR (400 MHz, DMSO-d₆) δ = 8.34 (d, J = 4.8 Hz, 1H), 7.82 (s, 1H), 7.30 (dd, J = 7.2, 8.8 Hz, 2H), 7.11 (d, J = 4.4 Hz, 1H), 5.46 (t, J = 5.6 Hz, 1H), 4.52 (d, J = 5.6 Hz, 2H), 4.29 (s, 4H), 4.00 (s, 2H), 2.39 (s, 3H). ¹⁹F NMR (376 MHz, DMSO- d₆) δ = −135.362, −164.623. |
| | 3-(hydroxymethyl)-1-methyl-7- | | |
| | (4-(3,4,5-trifluorobenzyl)pyridin- | | |
| | 2-yl)-6,7-dihydroimidazo[1,5- | | |
| | α]pyrazin-8(5H)-one | | |
| 145 | 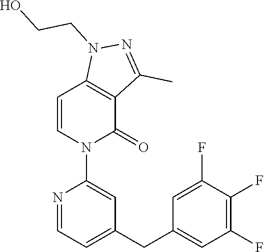 [SMILES: Cc1nn(CCO)c2ccn(-c3cc(Cc4cc(F)c(F)c(F)c4)ccn3)c(=O)c12] | 415.1 | ¹H NMR (400 MHz, DMSO-d₆) δ = 8.49 (d, J = 5.2 Hz, 1H), 7.73 (s, 1H), 7.67 (d, J = 7.6 Hz, 1H), 7.41-7.27 (m, 3H), 6.72 (d, J = 7.6 Hz, 1H), 4.88 (t, J = 5.2 Hz, 1H), 4.25 (t, J = 5.2 Hz, 2H), 4.07 (s, 2H), 3.75 (qd, J = 5.2, 10.4 Hz, 2H), 2.49 (s, 3H). |
| | 1-(2-hydroxyethyl)-3-methyl-5- | | |
| | (4-(3,4,5-trifluorobenzyl)pyridin- | | |
| | 2-yl)-1,5-dihydro-4H- | | |
| | pyrazolo[4,3-c]pyridin-4-one | | |
| 146 | 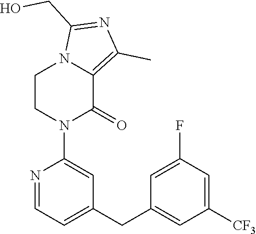 [SMILES: Cc1nc(CO)n2c1C(=O)N(c1cc(Cc3cc(F)cc(C(F)(F)F)c3)ccn1)CC2] | 435.2 | 1H NMR (400 MHz, DMSO-d6) δ = 8.35 (d, J = 4.8 Hz, 1H), 7.85 (s, 1H), 7.59 (s, 1H), 7.54 (dd, J = 6.8, 8.4 Hz, 2H), 7.15- 7.11 (m, 1H), 5.45 (t, J = 5.6 Hz, 1H), 4.51 (d, J = 5.6 Hz, 2H), 4.29 (s, 4H), 4.14 (s, 2H), 2.38 (s, 3H). |
| | 7-(4-(3-fluoro-5- | | |
| | (trifluoromethyl)benzyl)pyridin- | | |
| | 2-yl)-3-(hydroxymethyl)-1- | | |
| | methyl-6,7-dihydroimidazo[1,5- | | |
| | α]pyrazin-8(5H)-one | | |
| 147 | 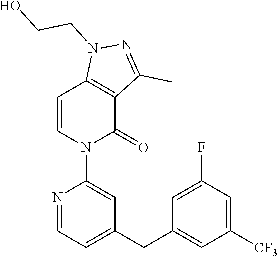 [SMILES: Cc1nn(CCO)c2ccn(-c3cc(Cc4cc(F)cc(C(F)(F)F)c4)ccn3)c(=O)c12] | 447.1 | ¹H NMR (400 MHz, DMSO-d₆) δ = 8.49 (d, J = 5.2 Hz, 1H), 7.76 (s, 1H), 7.71-7.61 (m, 2H), 7.56 (br dd, J = 6.0, 9.2 Hz, 2H), 7.38 (d, J = 5.2 Hz, 1H), 6.71 (d, J = 7.6 Hz, 1H), 4.87 (t, J = 5.6 Hz, 1H), 4.32-4.13 (m, 4H), 3.74 (qd, J = 5.2, 10.4 Hz, 2H), 2.48 (s, 3H) ¹⁹F NMR (376 MHz, DMSO- d₆) δ = −61.055, −110.553. |
| | 5-(4-(3-fluoro-5- | | |
| | (trifluoromethyl)benzyl)pyridin- | | |
| | 2-yl)-1-(2-hydroxyethyl)-3- | | |
| | methyl-1,5-dihydro-4H- | | |
| | pyrazolo[4,3-c]pyridin-4-one | | |
| 148 | 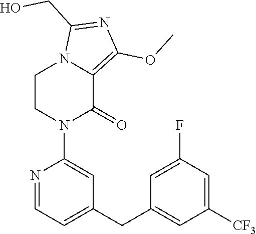 [SMILES: COc1nc(CO)n2c1C(=O)N(c1cc(Cc3cc(F)cc(C(F)(F)F)c3)ccn1)CC2] | 451.1 | 1H NMR (400 MHz, DMSO-d6) δ = 8.32 (d, J = 5.2 Hz, 1H), 7.78 (s, 1H), 7.60-7.49 (m, 3H), 7.10 (dd, J = 1.2, 5.2 Hz, 1H), 5.54-5.47 (m, 1H), 4.48 (d, J = 6.0 Hz, 2H), 4.27 (s, 4H), 4.11 (s, 2H), 3.88 (s, 3H). |
| | 7-(4-(3-fluoro-5- | | |
| | (trifluoromethyl)benzyl)pyridin- | | |
| | 2-yl)-3-(hydroxymethyl)-1- | | |
| | methoxy-6,7- | | |
| | dihydroimidazo[1,5-α]pyrazin- | | |
| | 8(5H)-one | | |
| 149 | 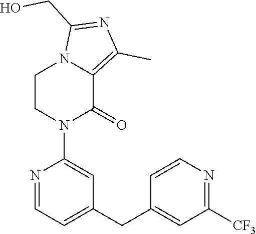 [SMILES: Cc1nc(CO)n2c1C(=O)N(c1cc(Cc3ccnc(C(F)(F)F)c3)ccn1)CC2] | 418.2 | ¹H NMR (400 MHz, DMSO-d₆) δ = 8.70 (d, J = 5.2 Hz, 1H), 8.37 (d, J = 5.2 Hz, 1H), 7.90- 7.88 (m, 1H), 7.88-7.86 (m, 1H), 7.63 (d, J = 4.8 Hz, 1H), 7.14 (dd, J = 1.6, 5.2 Hz, 1H), 5.49-5.42 (m, 1H), 4.51 (d, J = 4.4 Hz, 2H), 4.30 (s, 4H), 4.19 (s, 2H), 2.38 (s, 3H). |
| | 3-(hydroxymethyl)-1-methyl-7- | | |
| | (4-((2-(trifluoromethyl)pyridin- | | |
| | 4-yl)methyl)pyridin-2-yl)-6,7- | | |
| | dihydroimidazo[1,5-α]pyrazin- | | |
| | 8(5H)-one | | |
| 150 | 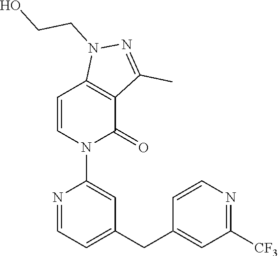 [SMILES: Cc1nn(CCO)c2ccn(-c3cc(Cc4ccnc(C(F)(F)F)c4)ccn3)c(=O)c12] | 430.2 | ¹H NMR (400 MHz, DMSO-d₆) δ = 8.70 (d, J = 4.8 Hz, 1H), 8.51 (d, J = 5.2 Hz, 1H), 7.93 (s, 1H), 7.78 (s, 1H), 7.71-7.64 (m, 2H), 7.40 (dd, J = 1.2, 5.2 Hz, 1H), 6.71 (d, J = 8.0 Hz, 1H), 4.99-4.79 (m, 1H), 4.28-4.21 (m, 4H), 3.74 (t, J = 5.2 Hz, 2H), 2.48 (s, 3H). |
| | 1-(2-hydroxyethyl)-3-methyl-5- | | |
| | (4-((2-(trifluoromethyl)pyridin- | | |
| | 4-yl)methyl)pyridin-2-yl)-1,5- | | |
| | dihydro-4H-pyrazolo[4,3- | | |
| | c]pyridin-4-one | | |
| 151 | 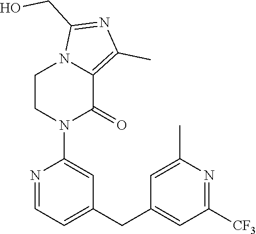 [SMILES: Cc1cc(Cc2ccnc(N3CCn4c(CO)nc(C)c4C3=O)c2)cc(C(F)(F)F)n1] | 432.2 | ¹H NMR (400 MHz, DMSO-d₆) δ = 8.36 (d, J = 5.2 Hz, 1H), 7.85 (s, 1H), 7.67 (s, 1H), 7.50 (s, 1H), 7.13 (d, J = 5.2 Hz, 1H), 5.46 (t, J = 5.6 Hz, 1H), 4.51 (d, J = 5.2 Hz, 2H), 4.30 (s, 4H), 4.13 (s, 2H), 2.52 (s, 3H), 2.38 (s, 3H). |
| | 3-(hydroxymethyl)-1-methyl-7- | | |
| | (4-((2-methyl-6- | | |
| | (trifluoromethyl)pyridin-4- | | |
| | yl)methyl)pyridin-2-yl)-6,7- | | |
| | dihydroimidazo[1,5-α]pyrazin- | | |
| | 8(5H)-one | | |
| 152 | 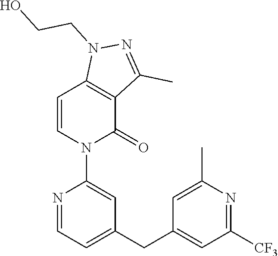 [SMILES: Cc1cc(Cc2ccnc(-n3ccc4c(c(C)nn4CCO)c3=O)c2)cc(C(F)(F)F)n1] | 444.2 | ¹H NMR (500 MHz, DMSO-d₆) δ = 8.50 (d, J = 5.5 Hz, 1H), 7.76 (s, 1H), 7.71 (s, 1H), 7.68 (d, J = 7.5 Hz, 1H), 7.53 (s, 1H), 7.38 (dd, J = 1.5, 5.0 Hz, 1H), 6.71 (d, J = 7.5 Hz, 1H), 4.87 (t, J = 5.5 Hz, 1H), 4.24 (t, J = 5.5 Hz, 2H), 4.19 (s, 2H), 3.75 (q, J = 5.5 Hz, 2H), 2.52 (s, 3H), 2.48 (s, 3H). |
| | 1-(2-hydroxyethyl)-3-methyl-5- | | |
| | (4-((2-methyl-6- | | |
| | (trifluoromethyl)pyridin-4- | | |
| | yl)methyl)pyridin-2-yl)-1,5- | | |
| | dihydro-4H-pyrazolo[4,3- | | |
| | c]pyridin-4-one | | |
| 154 | 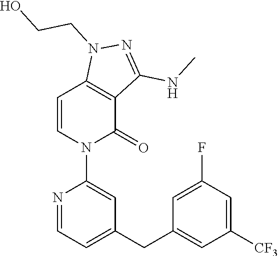 [SMILES: CNc1nn(CCO)c2ccn(-c3cc(Cc4cc(F)cc(C(F)(F)F)c4)ccn3)c(=O)c12] | 462.1 | ¹H NMR (500 MHz, DMSO-d₆) δ = 8.48 (d, J = 5.5 Hz, 1H), 7.73 (s, 1H), 7.65-7.60 (m, 2H), 7.55 ( t, J = 9.5 Hz, 2H), 7.36 (dd, J = 1.0, 4.0 Hz, 1H), 6.56 (d, J = 7.5 Hz, 1H), 5.55 (q, J = 5.0 Hz, 1H), 4.81 (t, J = 5.0 Hz, 1H), 4.18 (s, 2H), 4.07 (t, J = 5.0 Hz, 2H), 3.71 (q, J = 5.0 Hz, 2H), 2.81 (d, J = 5.0 Hz, 3H). |
| | 5-(4-(3-fluoro-5- | | |
| | (trifluoromethyl)benzyl)pyridin- | | |
| | 2-yl)-1-(2-hydroxyethyl)-3- | | |
| | (methylamino)-1,5-dihydro-4H- | | |
| | pyrazolo[4,3-c]pyridin-4-one | | |
| 155 | 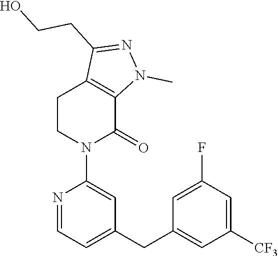 [SMILES: Cn1nc(CCO)c2c1C(=O)N(c1cc(Cc3cc(F)cc(C(F)(F)F)c3)ccn1)CC2] | 449.2 | ¹H NMR (400 MHz, DMSO-d₆) δ = 8.36 (d, J = 5.2 Hz, 1H), 7.69 (s, 1H), 7.58 (s, 1H), 7.53 (s, 2H), 7.16-7.12 (m, 1H), 4.67 (t, J = 5.2 Hz, 1H), 4.17 (t, J = 6.4 Hz, 2H), 4.13 (s, 2H), 4.01 (s, 3H), 3.64-3.58 (m, 2H), 2.81 (t, J = 6.4 Hz, 2H), 2.70 (t, J = 7.2 Hz, 2H). |
| | 6-(4-(3-fluoro-5- | | |
| | (trifluoromethyl)benzyl)pyridin- | | |
| | 2-yl)-3-(2-hydroxyethyl)-1- | | |
| | methyl-1,4,5,6-tetrahydro-7H- | | |
| | pyrazolo[3,4-c]pyridin-7-one | | |
| 156 | 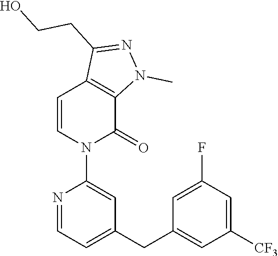 [SMILES: Cn1nc(CCO)c2ccn(-c3cc(Cc4cc(F)cc(C(F)(F)F)c4)ccn3)c(=O)c21] | 447.2 | ¹H NMR (400 MHz, DMSO-d₆) δ = 8.52 (d, J = 5.2 Hz, 1H), 7.77 (s, 1H), 7.64 (s, 1H), 7.56 (br d, J = 8.0 Hz, 2H), 7.42 (dd, J = 1.2, 4.8 Hz, 1H), 7.38 (d, J = 7.2 Hz, 1H), 6.70 (d, J = 7.2 Hz, 1H), 4.73 (t, J = 5.2 Hz, 1H), 4.21 (s, 5H), 3.79-3.64 (m, 2H), 2.93 (t, J = 6.8 Hz, 2H). |
| | 6-(4-(3-fluoro-5- | | |
| | (trifluoromethyl)benzyl)pyridin- | | |
| | 2-yl)-3-(2-hydroxyethyl)-1- | | |
| | methyl-1,6-dihydro-7H- | | |
| | pyrazolo[3,4-c]pyridin-7-one | | |
| 157 | 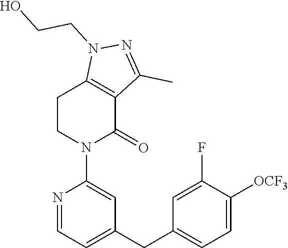 [SMILES: Cc1nn(CCO)c2c1C(=O)N(c1cc(Cc3ccc(OC(F)(F)F)c(F)c3)ccn1)CC2] | 465.2 | ¹H NMR (400 MHz, DMSO-d₆) δ = 8.32 (d, J = 5.2 Hz, 1H), 7.66 (s, 1H), 7.55-7.44 (m, 2H), 7.23 (d, J = 8.4 Hz, 1H), 7.06 (dd, J = 1.2, 4.8 Hz, 1H), 4.92 (t, J = 5.6 Hz, 1H), 4.21 (t, J = 6.0 Hz, 2H), 4.08-4.01 (m, 4H), 3.70 (q, J = 5.2 Hz, 2H), 3.04 (t, J = 6.4 Hz, 2H), 2.34 (s, 3H). |
| | 5-(4-(3-fluoro-4- | | |
| | (trifluoromethoxy)benzyl)pyridin- | | |
| | 2-yl)-1-(2-hydroxyethyl)-3- | | |
| | methyl-1,5,6,7-tetrahydro-4H- | | |
| | pyrazolo[4,3-c]pyridin-4-one | | |
| 158 | 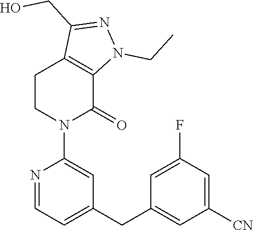 [SMILES: CCn1nc(CO)c2c1C(=O)N(c1cc(Cc3cc(F)cc(C#N)c3)ccn1)CC2] | 406.1 | ¹H NMR (400 MHz, DMSO-d₆) δ = 8.37 (d, J = 5.2 Hz, 1H), 7.76-7.69 (m, 3H), 7.60 (br d, J = 9.6 Hz, 1H), 7.17-7.13 (m, 1H), 5.10 (t, J = 5.6 Hz, 1H), 4.49-4.43 (m, 4H), 4.18 (t, J = 6.8 Hz, 2H), 4.09 (s, 2H), 2.93- 2.84 (m, 2H), 1.33 (t, J = 7.2 Hz, 3H). |
| | 3-((2-(1-ethyl-3- | | |
| | (hydroxymethyl)-7-oxo-1,4,5,7- | | |
| | tetrahydro-6H-pyrazolo[3,4- | | |
| | c]pyridin-6-yl)pyridin-4- | | |
| | yl)methyl)-5-fluorobenzonitrile | | |
| 159 | 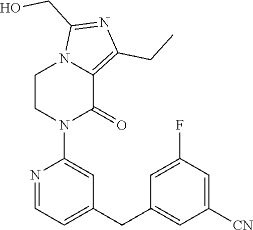 [SMILES: CCc1nc(CO)n2c1C(=O)N(c1cc(Cc3cc(F)cc(C#N)c3)ccn1)CC2] | 406.2 | ¹H NMR (500 MHz, DMSO-d₆) δ = 8.35 (d, J = 5.0 Hz, 1H), 7.84 (s, 1H), 7.75-7.66 (m, 2H), 7.59 (br d, J = 9.0 Hz, 1H), 7.13 (d, J = 5.0 Hz, 1H), 5.47 (br s, 1H), 4.53 (s, 2H), 4.29 (s, 4H), 4.09 (s, 2H), 2.83 (q, J = 7.5 Hz, 2H), 1.16 (t, J = 7.5 Hz, 3H). |
| | 3-((2-(1-ethyl-3- | | |
| | (hydroxymethyl)-8-oxo-5,6- | | |
| | dihydroimidazo[1,5-α]pyrazin- | | |
| | 7(8H)-yl)pyridin-4-yl)methyl)-5- | | |
| | fluorobenzonitrile | | |
| 160 | 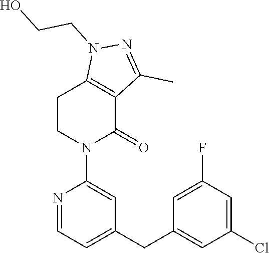 [SMILES: Cc1nn(CCO)c2c1C(=O)N(c1cc(Cc3cc(F)cc(Cl)c3)ccn1)CC2] | 415.1 | ¹H NMR (400 MHz, DMSO-d₆) δ = 8.31 (d, J = 5.2 Hz, 1H), 7.64 (s, 1H), 7.28 (td, J = 2.0, 8.8 Hz, 1H), 7.25 (s, 1H), 7.18 (dd, J = 1.6, 9.6 Hz, 1H), 7.06 (dd, J = 1.6, 5.2 Hz, 1H), 4.90 (t, J = 5.2 Hz, 1H), 4.20 (t, J = 6.8 Hz, 2H), 4.04 (t, J = 5.2 Hz, 2H), 4.00 (s, 2H), 3.69 (g, J = 5.2 Hz, 2H), 3.04 (t, J = 6.8 Hz, 2H), 2.34 (s, 3H). |
| | 5-(4-(3-chloro-5- | | |
| | fluorobenzyl)pyridin-2-yl)-1-(2- | | |
| | hydroxyethyl)-3-methyl-1,5,6,7- | | |
| | tetrahydro-4H-pyrazolo[4,3- | | |
| | c]pyridin-4-one | | |
| 161 | 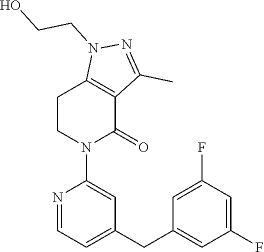 [SMILES: Cc1nn(CCO)c2c1C(=O)N(c1cc(Cc3cc(F)cc(F)c3)ccn1)CC2] | 399.2 | ¹H NMR (400 MHz, DMSO-d₆) δ = 8.31 (d, J = 4.8 Hz, 1H), 7.64 (s, 1H), 7.14-7.00 (m, 4H), 4.90 (t, J = 5.6 Hz, 1H), 4.19 (t, J = 7.2 Hz, 2H), 4.04 (t, J = 5.2 Hz, 2H), 4.00 (s, 2H), 3.69 (q, J = 5.2 Hz, 2H), 3.03 (t, J = 6.4 Hz, 2H), 2.34 (s, 3H). |
| | 5-(4-(3,5-difluorobenzyl)pyridin- | | |
| | 2-yl)-1-(2-hydroxyethyl)-3- | | |
| | methyl-1,5,6,7-tetrahydro-4//- | | |
| | pyrazolo[4,3-c]pyridin-4-one | | |
| 162 | 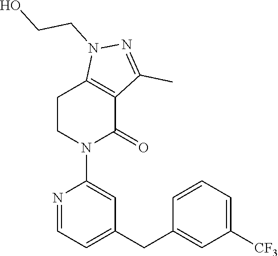 [SMILES: Cc1nn(CCO)c2c1C(=O)N(c1cc(Cc3cccc(C(F)(F)F)c3)ccn1)CC2] | 431.2 | ¹H NMR (400 MHz, DMSO-d₆) δ = 8.31 (d, J = 5.2 Hz, 1H), 7.67 (d, J = 11.6 Hz, 2H), 7.61- 7.52 (m, 3H), 7.04 (dd, J = 1.2, 5.2 Hz, 1H), 4.91 (t, J = 5.2 Hz, 1H), 4.19 (t, J = 6.4 Hz, 2H), 4.09 (s, 2H), 4.04 (t, J = 5.2 Hz, 2H), 3.69 (q, J = 4.8 Hz, 2H), 3.03 (t, J = 6.8 Hz, 2H), 2.33 (s, 3H). |
| | 1-(2-hydroxyethyl)-3-methyl-5- | | |
| | (4-(3- | | |
| | (trifluoromethyl)benzyl)pyridin- | | |
| | 2-yl)-1,5,6,7-tetrahydro-4H- | | |
| | pyrazolo[4,3-c]pyridin-4-one | | |
| 163 | 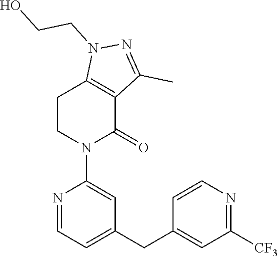 [SMILES: Cc1nn(CCO)c2c1C(=O)N(c1cc(Cc3ccnc(C(F)(F)F)c3)ccn1)CC2] | 432.2 | ¹H NMR (400 MHz, DMSO-d₆) δ = 8.69 (d, J = 5.2 Hz, 1H), 8.34 (d, J = 5.2 Hz, 1H), 7.89 (s, 1H), 7.69 (s, 1H), 7.62 (d, J = 4.8 Hz, 1H), 7.08 (dd, J = 1.2, 5.2 Hz, 1H), 4.91 (t, J = 5.2 Hz, 1H), 4.20 (t, J = 6.4 Hz, 2H), 4.16 (s, 2H), 4.04 (t, J = 5.2 Hz, 2H), 3.69 (q, J = 5.2 Hz, 2H), 3.03 (t, J = 6.4 Hz, 2H), 2.33 (s, 3H). |
| | 1-(2-hydroxyethyl)-3-methyl-5- | | |
| | (4-((2-(trifluoromethyl)pyridin- | | |
| | 4-yl)methyl)pyridin-2-yl)- | | |
| | 1,5,6,7-tetrahydro-4H- | | |
| | pyrazolo[4,3-c]pyridin-4-one | | |
| 164 | 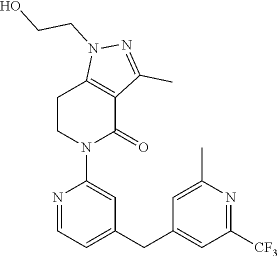 [SMILES: Cc1cc(Cc2ccnc(N3CCc4c(c(C)nn4CCO)C3=O)c2)cc(C(F)(F)F)n1] | 446.3 | ¹H NMR (400 MHz, DMSO-d₆) δ = 8.33 (d, J = 5.2 Hz, 1H), 7.67 (d, J = 4.4 Hz, 2H), 7.49 (s, 1H), 7.07 (dd, J = 1.2, 5.2 Hz, 1H), 4.92 (br s, 1H), 4.25-4.16 (m, 2H), 4.10 (s, 2H), 4.04 (t, J = 5.2 Hz, 2H), 3.69 (br d, J = 4.8 Hz, 2H), 3.09-2.99 (m, 2H), 2.51 (br s, 3H), 2.33 (s, 3H). |
| | 1-(2-hydroxyethyl)-3-methyl-5- | | |
| | (4-((2-methyl-6- | | |
| | (trifluoromethyl)pyridin-4- | | |
| | yl)methyl)pyridin-2-yl)-1,5,6,7- | | |
| | tetrahydro-4H-pyrazolo[4,3- | | |
| | c]pyridin-4-one | | |
| 165 | 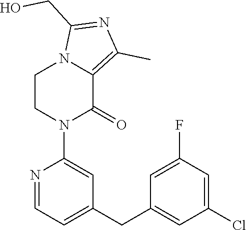 [SMILES: Cc1nc(CO)n2c1C(=O)N(c1cc(Cc3cc(F)cc(Cl)c3)ccn1)CC2] | 401.2 | ¹H NMR (400 MHz, DMSO-d₆) δ = 8.34 (d, J = 5.2 Hz, 1H), 7.82 (s, 1H), 7.29 (td, J = 2.0, 8.8 Hz, 1H), 7.26 (s, 1H), 7.19 (d, J = 9.6 Hz, 1H), 7.11 (dd, J = 1.2, 5.2 Hz, 1H), 5.45 (t, J = 5.6 Hz, 1H), 4.51 (d, J = 5.6 Hz, 2H), 4.29 (s, 4H), 4.03 (s, 2H), 2.39 (s, 3H). |
| | 7-(4-(3-chloro-5- | | |
| | fluorobenzyl)pyridin-2-yl)-3- | | |
| | (hydroxymethyl)-1-methyl-6,7- | | |
| | dihydroimidazo[1,5-α]pyrazin- | | |
| | 8(5H)-one | | |
| 166 | 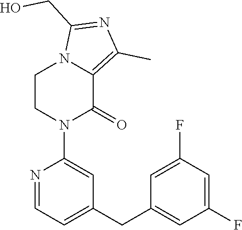 [SMILES: Cc1nc(CO)n2c1C(=O)N(c1cc(Cc3cc(F)cc(F)c3)ccn1)CC2] | 385.2 | ¹H NMR (400 MHz, DMSO-d₆) δ = 8.34 (d, J = 4.8 Hz, 1H), 7.82 (s, 1H), 7.14-7.02 (m, 4H), 5.46 (t, J = 5.2 Hz, 1H), 4.51 (d, J = 5.2 Hz, 2H), 4.29 (s, 4H), 4.03 (s, 2H), 2.38 (s, 3H). |
| | 7-(4-(3,5-difluorobenzyl)pyridin- | | |
| | 2-yl)-3-(hydroxymethyl)-1- | | |
| | methyl-6,7-dihydroimidazo[1,5- | | |
| | α]pyrazin-8(5H)-one | | |
| 167 | 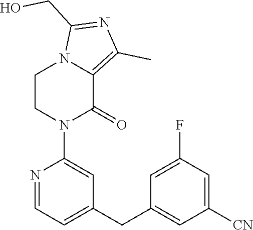 [SMILES: Cc1nc(CO)n2c1C(=O)N(c1cc(Cc3cc(F)cc(C#N)c3)ccn1)CC2] | 392.1 | ¹H NMR (400 MHz, DMSO-d₆) δ = 8.35 (d, J = 4.0 Hz, 1H), 7.83 (s, 1H), 7.76-7.67 (m, 2H), 7.59 (br d, J = 8.0 Hz, 1H), 7.13 (dd, J = 0.8, 4.0 Hz, 1H), 5.46 (t, J = 4.8 Hz, 1H), 4.51 (d, J = 4.4 Hz, 2H), 4.29 (s, 4H), 4.09 (s, 2H), 2.39 (s, 3H). |
| | 3-fluoro-5-((2-(3- | | |
| | (hydroxymethyl)-1-methyl-8- | | |
| | oxo-5,6-dihydroimidazo[1,5- | | |
| | α]pyrazin-7(8H)-yl)pyridin-4- | | |
| | yl)methyl)benzonitrile | | |
| 168 | 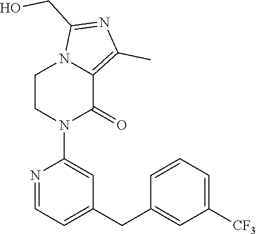 [SMILES: Cc1nc(CO)n2c1C(=O)N(c1cc(Cc3cccc(C(F)(F)F)c3)ccn1)CC2] | 417.1 | ¹H NMR (400 MHz, DMSO-d₆) δ = 8.34 (d, J = 5.2 Hz, 1H), 7.83 (s, 1H), 7.69 (s, 1H), 7.63- 7.52 (m, 3H), 7.10 (dd, J = 1.2, 1.6 Hz, 1H), 5.45 (t, J = 6.0 Hz, 1H), 4.51 (d, J = 6.0 Hz, 2H), 4.29 (s, 3H), 4.12 (s, 2H), 2.38 (s, 3H). |
| | 3-(hydroxymethyl)-1-methyl-7- | | |
| | (4-(3- | | |
| | (trifluoromethyl)benzyl)pyridin- | | |
| | 2-yl)-6,7-dihydroimidazo[1,5- | | |
| | α]pyrazin-8(5H)-one | | |
| 169 | 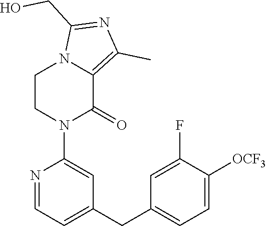 [SMILES: Cc1nc(CO)n2c1C(=O)N(c1cc(Cc3ccc(OC(F)(F)F)c(F)c3)ccn1)CC2] | 451.2 | ¹H NMR (400 MHz, DMSO-d₆) δ = 8.35 (d, J = 4.0 Hz, 1H), 7.84 (s, 1H), 7.55-7.45 (m, 2H), 7.24 (d, J = 6.8 Hz, 1H), 7.12 (dd, J = 0.8, 4.0 Hz, 1H), 5.46 (t, J = 4.8 Hz, 1H), 4.52 (d, J = 4.8 Hz, 2H), 4.30 (s, 4H), 4.06 (s, 2H), 2.39 (s, 3H). |
| | 7-(4-(3-fluoro-4- | | |
| | (trifluoromethoxy)benzyl)pyridin- | | |
| | 2-yl)-3-(hydroxymethyl)-1- | | |
| | methyl-6,7-dihydroimidazo[1,5- | | |
| | α]pyrazin-8(5H)-one | | |
| 170 | 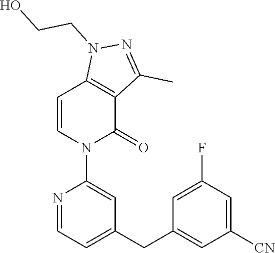 [SMILES: Cc1nn(CCO)c2ccn(-c3cc(Cc4cc(F)cc(C#N)c4)ccn3)c(=O)c12] | 404.1 | ¹H NMR (400 MHz, DMSO-d₆) δ = 8.49 (d, J = 4.0 Hz, 1H), 7.76-7.70 (m, 3H), 7.68-7.60 (m, 2H), 7.38 (d, J = 3.6 Hz, 1H), 6.71 (d, J = 6.4 Hz, 1H), 5.06-4.72 (m, 1H), 4.24 (t, J = 4.0 Hz, 2H), 4.14 (s, 2H), 3.74 (br t, J = 4.4 Hz, 2H), 2.48 (s, 3H). |
| | 3-fluoro-5-((2-(1-(2- | | |
| | hydroxyethyl)-3-methyl-4-oxo- | | |
| | 1,4-dihydro-5H-pyrazolo[4,3- | | |
| | c]pyridin-5-yl)pyridin-4- | | |
| | yl)methyl)benzonitrile | | |
| 171 | 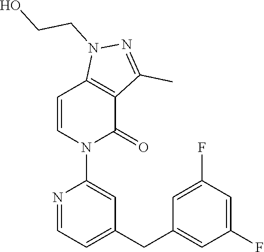 [SMILES: Cc1nn(CCO)c2ccn(-c3cc(Cc4cc(F)cc(F)c4)ccn3)c(=O)c12] | 397.2 | ¹H NMR (400 MHz, DMSO-d₆) δ = 8.49 (d, J = 5.2 Hz, 1H), 7.72 (s, 1H), 7.67 (d, J = 7.6 Hz, 1H), 7.37 (br d, J = 4.8 Hz, 1H), 7.10 (br d, J = 5.6 Hz, 3H), 6.71 (d, J = 7.6 Hz, 1H), 4.88 (t, J = 5.2 Hz, 1H), 4.24 (br t, J = 4.8 Hz, 2H), 4.09 (s, 2H), 3.74 (q, J = 5.2 Hz, 2H), 2.48 (s, 3H). |
| | 5-(4-(3,5-difluorobenzyl)pyridin- | | |
| | 2-yl)-1-(2-hydroxyethyl)-3- | | |
| | methyl-1,5-dihydro-4H- | | |
| | pyrazolo[4,3-c]pyridin-4-one | | |
| 172 | 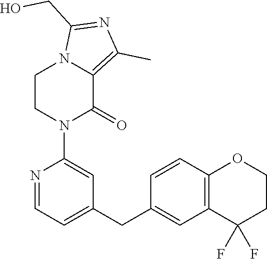 [SMILES: Cc1nc(CO)n2c1C(=O)N(c1cc(Cc3ccc4c(c3)C(F)(F)CCO4)ccn1)CC2] | 441.2 | ¹H NMR (400 MHz, CDCl₃) δ = 8.29 (d, J = 5.2 Hz, 1H), 7.91 (s, 1H), 7.42 (s, 1H), 7.17 ( d, J = 8.8 Hz, 1H), 6.88-6.82 (m, 2H), 4.73 (s, 2H), 4.48-4.45 (m, 2H), 4.34-4.30 (m, 4H), 3.96 (s, 2H), 2.56 (s, 3H), 2.48-2.41 (m, 2H). ¹⁹F NMR (376 MHz, DMSO- d₆) δ = − 85.954. |
| | 7-(4-((4,4-difluorochroman-6- | | |
| | yl)methyl)pyridin-2-yl)-3- | | |
| | (hydroxymethyl)-1-methyl-6,7- | | |
| | dihydroimidazo[1,5-α]pyrazin- | | |
| | 8(5H)-one | | |
| 173 | 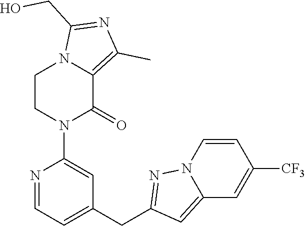 [SMILES: Cc1nc(CO)n2c1C(=O)N(c1cc(Cc3cc4cc(C(F)(F)F)ccn4n3)ccn1)CC2] | 457.1 | ¹H NMR (400 MHz, DMSO-d₆) δ = 8.83 (d, J = 7.2 Hz, 1H), 8.35 (d, J = 4.8 Hz, 1H), 8.18 (s, 1H), 7.85 (s, 1H), 7.14 (dd, J = 1.2, 5.2 Hz, 1H), 7.06 (dd, J = 2.0, 7.2 Hz, 1H), 6.74 (s, 1H), 5.45 (t, J = 6.0 Hz, 1H), 4.51 (d, J = 5.6 Hz, 2H), 4.29 (s, 4H), 4.23 (s, 2H), 2.37 (s, 3H). |
| | 3-(hydroxymethyl)-1-methyl-7- | | |
| | (4-((5- | | |
| | (trifluoromethyl)pyrazolo[1,5- | | |
| | a]pyridin-2-yl)methyl)pyridin-2- | | |
| | yl)-6,7-dihydroimidazo[1,5- | | |
| | α]pyrazin-8(5H)-one | | |
| 175 | 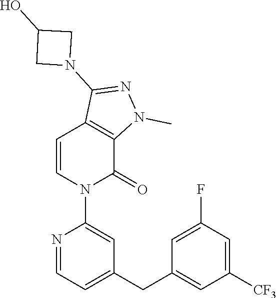 [SMILES: Cn1nc(N2CC(O)C2)c2ccn(-c3cc(Cc4cc(F)cc(C(F)(F)F)c4)ccn3)c(=O)c21] | 474.0 | ¹H NMR (400 MHz, DMSO-d₆) δ = 8.51 (d, J = 5.2 Hz, 1H), 7.76 (s, 1H), 7.64 (s, 1H), 7.60- 7.53 (m, 2H), 7.44-7.40 (m, 1H), 7.25 (d, J = 7.6 Hz, 1H), 6.52 (d, J = 7.2 Hz, 1H), 5.66 (d, J = 6.8 Hz, 1H), 4.63-4.52 (m, 1H), 4.26-4.16 (m, 4H), 4.09 (s, 3H), 3.76-3.67 (m, 2H). |
| | 6-(4-(3-fluoro-5- | | |
| | (trifluoromethyl)benzyl)pyridin- | | |
| | 2-yl)-3-(3-hydroxyazetidin-1-yl)- | | |
| | 1-methyl-1,6-dihydro-7H- | | |
| | pyrazolo[3,4-c]pyridin-7-one | | |
| 176 | 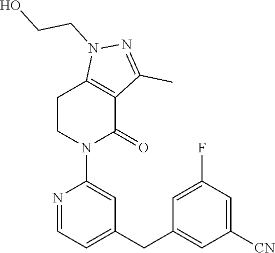 [SMILES: Cc1nn(CCO)c2c1C(=O)N(c1cc(Cc3cc(F)cc(C#N)c3)ccn1)CC2] | 406.2 | ¹H NMR (400 MHz, DMSO-d₆) δ = 8.32 (d, J = 5.2 Hz, 1H), 7.75-7.68 (m, 2H), 7.65 (s, 1H), 7.58 (br d, J = 10.0 Hz, 1H), 7.10-7.04 (m, 1H), 4.92 (t, J = 5.6 Hz, 1H), 4.19 (t, J = 6.4 Hz, 2H), 4.07-3.99 (m, 4H), 3.69 (q, J = 5.6 Hz, 2H), 3.03 (t, J = 6.4 Hz, 2H), 2.33 (s, 3H). |
| | 3-fluoro-5-((2-(1-(2- | | |
| | hydroxyethyl)-3-methyl-4-oxo- | | |
| | 1,4,6,7-tetrahydro-5H- | | |
| | pyrazolo[4,3-c]pyridin-5- | | |
| | yl)pyridin-4- | | |
| | yl)methyl)benzonitrile | | |
| 177 | 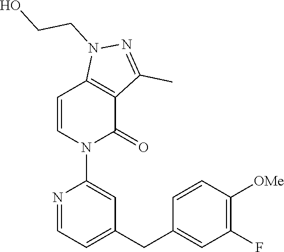 [SMILES: COc1ccc(Cc2ccnc(-n3ccc4c(c(C)nn4CCO)c3=O)c2)cc1F] | 409.2 | ¹H NMR (400 MHz, DMSO-d₆) δ = 8.46 (d, J = 5.2 Hz, 1H), 7.68-7.62 (m, 2H), 7.31 (dd, J = 1.2, 5.2 Hz, 1H), 7.18 (dd, J = 2.0, 12.8 Hz, 1H), 7.13-7.04 (m, 2H), 6.70 (d, J = 7.6 Hz, 1H), 4.88 (t, J = 5.6 Hz, 1H), 4.23 (t, J = 5.2 Hz, 2H), 3.99 (s, 2H), 3.80 (s, 3H), 3.74 (q, J = 5.2 Hz, 2H), 2.48 (s, 3H). |
| | 5-(4-(3-fluoro-4- | | |
| | methoxybenzyl)pyridin-2-yl)-1- | | |
| | (2-hydroxyethyl)-3-methyl-1,5- | | |
| | dihydro-4H-pyrazolo[4,3- | | |
| | c]pyridin-4-one | | |
| 178 | 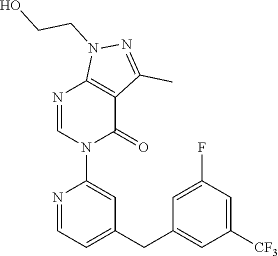 [SMILES: Cc1nn(CCO)c2ncn(-c3cc(Cc4cc(F)cc(C(F)(F)F)c4)ccn3)c(=O)c12] | 448.2 | ¹H NMR (400 MHz, DMSO-d₆) δ = 8.55 (d, J = 5.2 Hz, 1H), 8.47 (s, 1H), 7.73 (s, 1H), 7.65 (s, 1H), 7.59-7.54 (m, 2H), 7.51 (d, J = 5.2 Hz, 1H), 4.89 (t, J = 5.6 Hz, 1H), 4.28 (t, J = 5.6 Hz, 2H), 4.22 (s, 2H), 3.79 (q, J = 5.6 Hz, 2H), 2.46 (s, 3H). |
| | 5-(4-(3-fluoro-5- | | |
| | (trifluoromethyl)benzyl)pyridin- | | |
| | 2-yl)-1-(2-hydroxyethyl)-3- | | |
| | methyl-1,5-dihydro-4H- | | |
| | pyrazolo[3,4-d]pyrimidin-4-one | | |
| 179 | 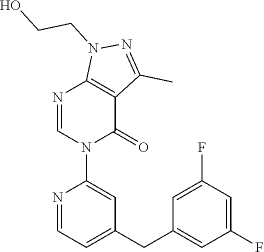 [SMILES: Cc1nn(CCO)c2ncn(-c3cc(Cc4cc(F)cc(F)c4)ccn3)c(=O)c12] | 398.2 | ¹H NMR (400 MHz, DMSO-d₆) δ = 8.54 (d, J = 5.2 Hz, 1H), 8.47 (s, 1H), 7.70 (s, 1H), 7.48 (d, J = 5.2 Hz, 1H), 7.15-7.05 (m, 3H), 4.90 (t, J = 5.6 Hz, 1H), 4.28 (t, J = 5.6 Hz, 2H), 4.11 (s, 2H), 3.79 (q, J = 5.6 Hz, 2H), 2.46 (s, 3H). |
| | 5-(4-(3,5-difluorobenzyl)pyridin- | | |
| | 2-yl)-1-(2-hydroxyethyl)-3- | | |
| | methyl-1,5-dihydro-4H- | | |
| | pyrazolo[3,4-d]pyrimidin-4-one | | |
| 180 | 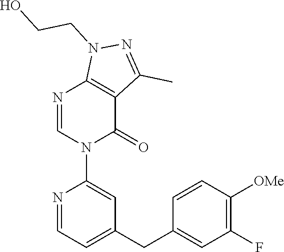 [SMILES: COc1ccc(Cc2ccnc(-n3cnc4c(c(C)nn4CCO)c3=O)c2)cc1F] | 410.3 | ¹H NMR (400 MHz, DMSO-d₆) δ = 8.51 (d, J = 5.2 Hz, 1H), 8.46 (s, 1H), 7.63 (s, 1H), 7.42 (d, J = 4.4 Hz, 1H), 7.19 (d, J = 11.6 Hz, 1H), 7.14-7.06 (m, 2H), 4.89 (t, J = 5.2 Hz, 1H), 4.27 (t, J = 5.6 Hz, 2H), 4.01 (s, 2H), 3.81-3.77 (m, 5H), 2.46 (s, 3H). |
| | 5-(4-(3-fluoro-4- | | |
| | methoxybenzyl)pyridin-2-yl)-1- | | |
| | (2-hydroxyethyl)-3-methyl-1,5- | | |
| | dihydro-4H-pyrazolo[3,4- | | |
| | d]pyrimidin-4-one | | |
| 181 | 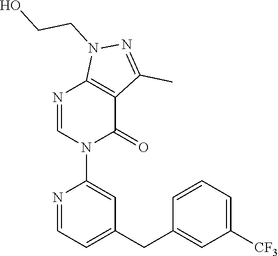 [SMILES: Cc1nn(CCO)c2ncn(-c3cc(Cc4cccc(C(F)(F)F)c4)ccn3)c(=O)c12] | 430.2 | ¹H NMR (400 MHz, DMSO-d₆) δ = 8.54 (d, J = 4.8 Hz, 1H), 8.47 (s, 1H), 7.75 (s, 1H), 7.70 (s, 1H), 7.64-7.56 (m, 3H), 7.47 (d, J = 5.2 Hz, 1H), 4.89 (t, J = 7.0 Hz, 1H), 4.27 (t, J = 5.6 Hz, 2H), 4.20 (s, 2H), 3.81-3.76 (m, 2H), 2.46 (s, 3H). |
| | 1-(2-hydroxyethyl)-3-methyl-5- | | |
| | (4-(3- | | |
| | (trifluoromethyl)benzyl)pyridin- | | |
| | 2-yl)-1,5-dihydro-4H- | | |
| | pyrazolo[3,4-d]pyrimidin-4-one | | |
| 183 | 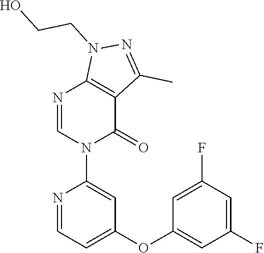 [SMILES: Cc1nn(CCO)c2ncn(-c3cc(Oc4cc(F)cc(F)c4)ccn3)c(=O)c12] | 400.1 | ¹H NMR (400 MHz, DMSO-d₆) δ = 8.56 (d, J = 5.6 Hz, 1H), 8.46 (s, 1H), 7.43 (d, J = 2.0 Hz, 1H), 7.29-7.18 (m, 2H), 7.17- 7.05 (m, 2H), 4.90 (t, J = 5.6 Hz, 1H), 4.27 (t, J = 5.6 Hz, 2H), 3.79 (q, J = 5.5 Hz, 2H), 2.48-2.40 (m, 3H). |
| | 5-(4-(3,5- | | |
| | difluorophenoxy)pyridin-2-yl)-1- | | |
| | (2-hydroxyethyl)-3-methyl-1,5- | | |
| | dihydro-4H-pyrazolo[3,4- | | |
| | d]pyrimidin-4-one | | |
| 184 | 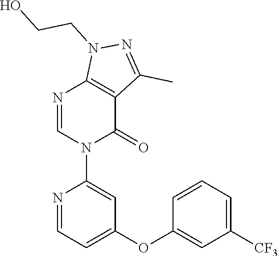 [SMILES: Cc1nn(CCO)c2ncn(-c3cc(Oc4cccc(C(F)(F)F)c4)ccn3)c(=O)c12] | 432.2 | ¹H NMR (400 MHz, CDCl₃) δ = 8.55 (d, J = 5.6 Hz, 1H), 8.47 (s, 1H), 7.79-7.73 (m, 1H), 7.72-7.68 (m, 1H), 7.65 (s, 1H), 7.58 (d, J = 8.0 Hz, 1H), 7.38 (s, 1H), 7.16 (d, J = 3.6 Hz, 1H), 4.88 (t, J = 5.2 Hz, 1H), 4.27 (t, J = 5.6 Hz, 2H), 3.79 (d, J = 5.4 Hz, 2H), 2.44 (s, 3H). |
| | 1-(2-hydroxyethyl)-3-methyl-5- | | |
| | (4-(3- | | |
| | (trifluoromethyl)phenoxy)pyridin- | | |
| | 2-yl)-1,5-dihydro-4H- | | |
| | pyrazolo[3,4-d]pyrimidin-4-one | | |
| 185 | 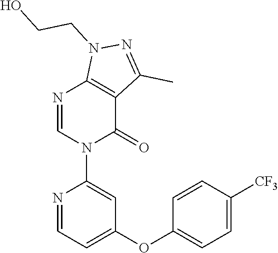 [SMILES: Cc1nn(CCO)c2ncn(-c3cc(Oc4ccc(C(F)(F)F)cc4)ccn3)c(=O)c12] | 432.1 | ¹H NMR (400 MHz, DMSO-d₆) δ = 8.57 (d, J = 5.6 Hz, 1H), 8.47 (s, 1H), 7.89 (d, J = 8.4 Hz, 2H), 7.47-7.40 (m, 3H), 7.27- 7.19 (m, 1H), 4.89 (t, J = 5.6 Hz, 1H), 4.27 (t, J = 5.6 Hz, 2H), 3.79 (q, J = 5.4 Hz, 2H), 2.44 (s, 3H). |
| | 1-(2-hydroxyethyl)-3-methyl-5- | | |
| | (4-(4- | | |
| | (trifluoromethyl)phenoxy)pyridin- | | |
| | 2-yl)-1,5-dihydro-4H- | | |
| | pyrazolo[3,4-d]pyrimidin-4-one | | |
| 186 | 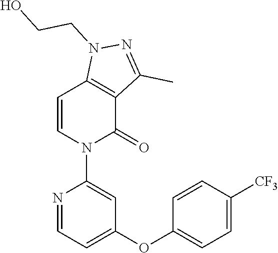 [SMILES: Cc1nn(CCO)c2ccn(-c3cc(Oc4ccc(C(F)(F)F)cc4)ccn3)c(=O)c12] | 431.1 | ¹H NMR (400 MHz, DMSO-d₆) δ = 8.54 (1H, d, J = 5.6 Hz), 7.89 (2H, br d, J = 8.4 Hz), 7.74 (1H, d, J-7.6 Hz), 7.41-7.50 (3H, m), 7.13 (1H, dd, J = 2.0 and 5.6 Hz), 6.74 (1H, d, J = 7.6 Hz), 4.89 (1H, t, J = 5.2 Hz), 4.24 (2H, br t, J = 5.2 Hz), 3.74 (2H, q, J = 5.2 Hz), 2.46 (3H, s). |
| | 1-(2-hydroxyethyl)-3-methyl-5- | | |
| | (4-(4- | | |
| | (trifluoromethyl)phenoxy)pyridin- | | |
| | 2-yl)-1,5-dihydro-4H- | | |
| | pyrazolo[4,3-c]pyridin-4-one | | |
| 187 | 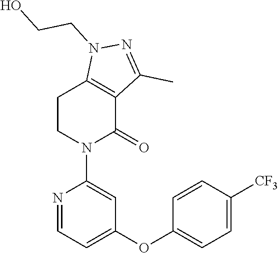 [SMILES: Cc1nn(CCO)c2c1C(=O)N(c1cc(Oc3ccc(C(F)(F)F)cc3)ccn1)CC2] | 433.1 | ¹H NMR (400 MHz, DMSO-d₆) δ = 8.37 (d, J = 5.6 Hz, 1H), 7.85 (d, J = 8.4 Hz, 2H), 7.42 (d, J = 2.0 Hz, 1H), 7.38 (d, J = 8.4 Hz, 2H), 6.89-6.85 (m, 1H), 4.92 (t, J = 5.6 Hz, 1H), 4.31- 4.22 (m, 2H), 4.07-4.01 (m, 2H), 3.68 (q, J = 5.2 Hz, 2H), 3.04 (t, J = 6.4 Hz, 2H), 2.30 (s, 3H). |
| | 1-(2-hydroxyethyl)-3-methyl-5- | | |
| | (4-(4- | | |
| | (trifluoromethyl)phenoxy)pyridin- | | |
| | 2-yl)-1,5,6,7-tetrahydro-4H- | | |
| | pyrazolo[4,3-c]pyridin-4-one | | |
| 188 | 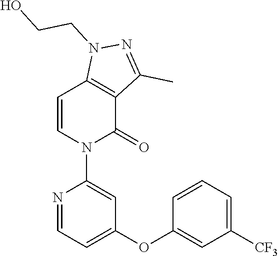 [SMILES: Cc1nn(CCO)c2ccn(-c3cc(Oc4cccc(C(F)(F)F)c4)ccn3)c(=O)c12] | 431.1 | ¹H NMR (400 MHz, DMSO-d₆) δ = 8.51 (d, J = 5.6 Hz, 1H), 7.78-7.62 (m, 4H), 7.57 (d, J = 8.0 Hz, 1H), 7.42 (d, J = 2.4 Hz, 1H), 7.05 (dd, J = 2.4, 5.6 Hz, 1H), 6.73 (d, J = 7.6 Hz, 1H), 4.92-4.72 (m, 1H), 4.23 (t, J = 5.2 Hz, 2H), 3.74 (q, J = 5.2 Hz, 2H), 2.46 (s, 3H). |
| | 1-(2-hydroxyethyl)-3-methyl-5- | | |
| | (4-(3- | | |
| | (trifluoromethyl)phenoxy)pyridi | | |
| | n-2-yl)-1,5-dihydro-4H- | | |
| | pyrazolo[4,3-c]pyridin-4-one | | |
| 189 | 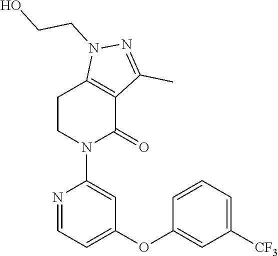 [SMILES: Cc1nn(CCO)c2c1C(=O)N(c1cc(Oc3cccc(C(F)(F)F)c3)ccn1)CC2] | 433.2 | ¹H NMR (400 MHz, DMSO-d₆) δ = 8.35 (d, J = 5.6 Hz, 1H), 7.76-7.69 (m, 1H), 7.68-7.62 (m, 1H), 7.58 (br s, 1H), 7.51 (br d, J = 7.6 Hz, 1H), 7.38 (s, 1H), 6.84-6.78 (m, 1H), 4.99- 4.88 (m, 1H), 4.29-4.21 (m, 2H), 4.08-4.00 (m, 2H), 3.74- 3.63 (m, 2H), 3.08-2.99 (m, 2H), 2.30 (s, 3H). |
| | 1-(2-hydroxyethyl)-3-methyl-5- | | |
| | (4-(3- | | |
| | (trifluoromethyl)phenoxy)pyridin- | | |
| | 2-yl)-1,5,6,7-tetrahydro-4H- | | |
| | pyrazolo[4,3-c]pyridin-4-one | | |
| 190 | 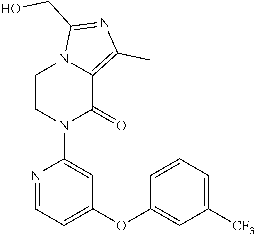 [SMILES: Cc1nc(CO)n2c1C(=O)N(c1cc(Oc3cccc(C(F)(F)F)c3)ccn1)CC2] | 419.2 | ¹H NMR (400 MHz, DMSO-d₆) δ = 8.37 (d, J = 5.6 Hz, 1H), 7.80-7.71 (m, 1H), 7.70-7.63 (m, 1H), 7.60 (s, 1H), 7.56 (d, J = 2.0 Hz, 1H), 7.53 (d, J = 8.0 Hz, 1H), 6.85 (dd, J = 2.4, 5.6 Hz, 1H), 5.47 (t, J = 5.6 Hz, 1H), 4.51 (d, J = 5.6 Hz, 2H), 4.39-4.32 (m, 2H), 4.32-4.26 (m, 2H), 2.35 (s, 3H). |
| | 3-(hydroxymethyl)-1-methyl-7- | | |
| | (4-(3- | | |
| | (trifluoromethyl)phenoxy)pyridin- | | |
| | 2-yl)-6,7-dihydroimidazo[1,5- | | |
| | α]pyrazin-8(5H)-one | | |
| 191 | 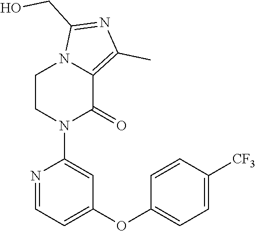 [SMILES: Cc1nc(CO)n2c1C(=O)N(c1cc(Oc3ccc(C(F)(F)F)cc3)ccn1)CC2] | 419.1 | ¹H NMR (400 MHz, DMSO-d₆) δ = 8.41 (1H, d, J = 5.6 Hz), 7.87 (2H, d, J = 8.68 Hz), 7.60 (1H, d, J = 2.0 Hz), 7.40 (2H, d, J = 8.54 Hz), 6.92 (1H, dd, J = 2.4, 5.6 Hz), 5.47 (1H, t, J = 6.0 Hz), 4.52 (2H, d, J = 6.0 Hz), 4.27-4.38 (4H, m), 2.36 (3H, s). |
| | 3-(hydroxymethyl)-1-methyl-7- | | |
| | (4-(4- | | |
| | (trifluoromethyl)phenoxy)pyridin- | | |
| | 2-yl)-6,7-dihydroimidazo[1,5- | | |
| | α]pyrazin-8(5H)-one | | |
| 192 | 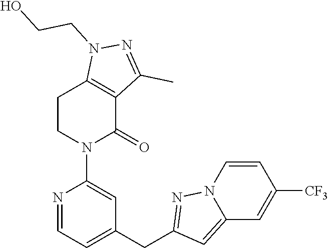 [SMILES: Cc1nn(CCO)c2c1C(=O)N(c1cc(Cc3cc4cc(C(F)(F)F)ccn4n3)ccn1)CC2] | 471.2 | ¹H NMR (400 MHz, DMSO-d₆) δ = 8.83 (d, J = 7.2 Hz, 1H), 8.31 (d, J = 5.2 Hz, 1H), 8.17 (s, 1H), 7.67 (s, 1H), 7.14-6.99 (m, 2H), 6.73 (s, 1H), 4.90 (t, J = 5.6 Hz, 1H), 4.24-4.15 (m, 4H), 4.04 (t, J = 5.2 Hz, 2H), 3.69 (q, J = 5.2 Hz, 2H), 3.03 (t, J = 6.8 Hz, 2H), 2.33 (s, 3H) |
| | 1-(2-hydroxyethyl)-3-methyl-5- | | |
| | (4-((5- | | |
| | (trifluoromethyl)pyrazolo[1,5- | | |
| | a]pyridin-2-yl)methyl)pyridin-2- | | |
| | yl)-1,5,6,7-tetrahydro-4H- | | |
| | pyrazolo[4,3-c]pyridin-4-one | | |
| 193 | 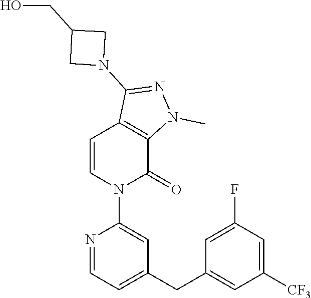 [SMILES: Cn1nc(N2CC(CO)C2)c2ccn(-c3cc(Cc4cc(F)cc(C(F)(F)F)c4)ccn3)c(=O)c21] | 490.1 | ¹H NMR (400 MHz, DMSO-d₆) δ = 8.36 (d, J = 5.2 Hz, 1H), 7.68 (s, 1H), 7.59 (s, 1H), 7.57- 7.49 (m, 2H), 7.14 (d, J = 4.8 Hz, 1H), 4.76-4.68 (m, 1H), 4.19-4.10 (m, 4H), 3.91 (s, 3H), 3.85 (t, J = 7.6 Hz, 2H), 3.60 (t, J = 6.4 Hz, 2H), 3.55 (t, J = 6.0 Hz, 2H), 2.80-2.71 (m, 3H). |
| | 6-(4-(3-fluoro-5- | | |
| | (trifluoromethyl)benzyl)pyridin- | | |
| | 2-yl)-3-(3- | | |
| | (hydroxymethyl)azetidin-1-yl)-1- | | |
| | methyl-1,4,5,6-tetrahydro-7H- | | |
| | pyrazolo[3,4-c]pyridin-7-one | | |
| 194 | 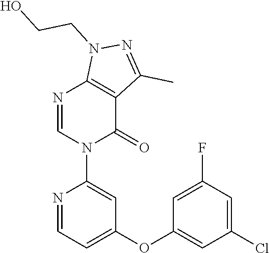 [SMILES: Cc1nn(CCO)c2ncn(-c3cc(Oc4cc(F)cc(Cl)c4)ccn3)c(=O)c12] | 416.1 | ¹H NMR (400 MHz, DMSO-d₆) δ = 8.56 (d, J = 5.6 Hz, 1H), 8.46 (s, 1H), 7.47-7.39 (m, 2H), 7.30-7.21 (m, 3H), 4.89 (t, J = 5.2 Hz, 1H), 4.27 (t, J = 5.6 Hz, 2H), 3.79 (q, J = 5.6 Hz, 2H), 2.45 (s, 3H). |
| | 5-(4-(3-chloro-5- | | |
| | fluorophenoxy)pyridin-2-yl)-1- | | |
| | (2-hydroxyethyl)-3-methyl-1,5- | | |
| | dihydro-4H-pyrazolo[3,4- | | |
| | d]pyrimidin-4-one | | |
| 196 | 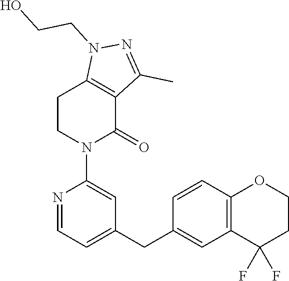 [SMILES: Cc1nn(CCO)c2c1C(=O)N(c1cc(Cc3ccc4c(c3)C(F)(F)CCO4)ccn1)CC2] | 455.3 | ¹H NMR (400 MHz, DMSO-d₆) δ = 8.29 (d, J = 4.8 Hz, 1H), 7.61 (s, 1H), 7.47 (s, 1H), 7.31 (br d, J = 8.4 Hz, 1H), 7.00 (dd, J = 1.2, 5.2 Hz, 1H), 6.90 (d, J = 8.4 Hz, 1H), 4.90 (t, J = 5.6 Hz, 1H), 4.32-4.25 (m, 2H), 4.19 (t, J = 6.4 Hz, 2H), 4.04 (t, J = 5.2 Hz, 2H), 3.95 (s, 2H), 3.69 (q, J = 5.2 Hz, 2H), 3.03 (br t, J = 6.8 Hz, 2H), 2.54 (br s, 2H), 2.33 (s, 3H). |
| | 5-(4-((4,4-difluorochroman-6- | | |
| | yl)methyl)pyridin-2-yl)-1-(2- | | |
| | hydroxyethyl)-3-methyl-1,5,6,7- | | |
| | tetrahydro-4H-pyrazolo[4,3- | | |
| | c]pyridin-4-one | | |
| 197 | 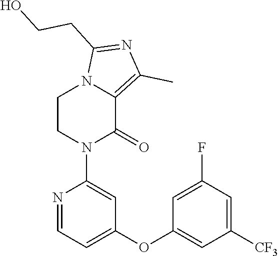 [SMILES: Cc1nc(CCO)n2c1C(=O)N(c1cc(Oc3cc(F)cc(C(F)(F)F)c3)ccn1)CC2] | 449.2 | ¹H NMR (400 MHz, DMSO-d6) δ = 8.34 (br d, J = 4.8 Hz, 1H), 7.83 (s, 1H), 7.63-7.47 (m, 3H), 7.12 (br d, J = 4.4 Hz, 1H), 4.82 (br t, J = 4.4 Hz, 1H), 4.31-4.17 (m, 4H), 4.13 (br s, 2H), 3.70 (br d, J = 5.6 Hz, 2H), 2.80 (br t, J = 5.6 Hz, 2H), 2.37 (s, 3H). |
| | 7-(4-(3-fluoro-5- | | |
| | (trifluoromethyl)benzyl)pyridin- | | |
| | 2-yl)-3-(2-hydroxyethyl)-1- | | |
| | methyl-6,7-dihydroimidazo[1,5- | | |
| | α]pyrazin-8(5H)-one | | |
| 198 | 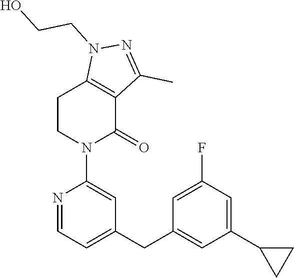 [SMILES: Cc1nn(CCO)c2c1C(=O)N(c1cc(Cc3cc(F)cc(C4CC4)c3)ccn1)CC2] | 421.2 | ¹H NMR (400 MHz, DMSO-d₆) δ = 8.29 (d, J = 5.2 Hz, 1H), 7.60 (s, 1H), 7.02 (d, J = 5.2 Hz, 1H), 6.91-6.88 (m, 1H), 6.84 (d, J = 9.6 Hz, 1H), 6.74-6.69 (m, 1H), 4.92 (t, J = 5.6 Hz, 1H), 4.19 (t, J = 6.4 Hz, 2H), 4.04 (t, J = 5.2 Hz, 2H), 3.92 (s, 2H), 3.69 (q, J = 5.2 Hz, 2H), 3.03 (t, J = 6.4 Hz, 2H), 2.34 (s, 3H), 1.94-1.86 (m, 1H), 0.98-0.91 (m, 2H), 0.73-0.67 (m, 2H). |
| | 5-(4-(3-cyclopropyl-5- | | |
| | fluorobenzyl)pyridin-2-yl)-1-(2- | | |
| | hydroxyethyl)-3-methyl-1,5,6,7- | | |
| | tetrahydro-4H-pyrazolo[4,3- | | |
| | c]pyridin-4-one | | |
| 199 | 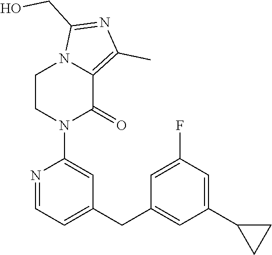 [SMILES: Cc1nc(CO)n2c1C(=O)N(c1cc(Cc3cc(F)cc(C4CC4)c3)ccn1)CC2] | 407.2 | ¹H NMR (400 MHz, DMSO-d₆) δ = 8.33 (d, J = 5.2 Hz, 1H), 7.79 (s, 1H), 7.08 (d, J = 5.2 Hz, 1H), 6.90 (s, 1H), 6.85 (d, J = 9.6 Hz, 1H), 6.76-6.69 (m, 1H), 5.47 (t, J = 5.6 Hz, 1H), 4.51 (d, J = 5.6 Hz, 2H), 4.29 (s, 4H), 3.95 (s, 2H), 2.38 (s, 3H), 1.94- 1.86 (m, 1H), 1.00-0.88 (m, 2H), 0.76-0.67 (m, 2H). |
| | 7-(4-(3-cyclopropyl-5- | | |
| | fluorobenzyl)pyridin-2-yl)-3- | | |
| | (hydroxymethyl)-1-methyl-6,7- | | |
| | dihydroimidazo[1,5-α]pyrazin- | | |
| | 8(5H)-one | | |
| 200 | 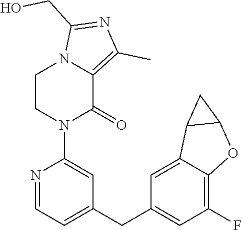 [SMILES: Cc1nc(CO)n2c1C(=O)N(c1cc(Cc3cc(F)c4c(c3)C3CC3O4)ccn1)CC2] | 421.0 | ¹H NMR (400 MHz, DMSO-d₆) δ = 8.33 (d, J = 5.2 Hz, 1H), 7.78 (s, 1H), 7.12 (s, 1H), 7.08 (d, J = 4.8 Hz, 1H), 6.97 (d, J = 11.6 Hz, 1H), 5.49 (t, J = 5.6 Hz, 1H), 5.05 (t, J = 4.4 Hz, 1H), 4.52 (d, J = 5.6 Hz, 2H), 4.29 (s, 4H), 3.92 (s, 2H), 2.78- 2.69 (m, 1H), 2.39 (s, 3H), 1.16-1.06 (m, 1H), 0.33-0.22 (m, 1H). |
| | 7-(4-((3-fluoro-1a,6b-dihydro- | | |
| | 1H-cyclopropa[b]benzofuran-5- | | |
| | yl)methyl)pyridin-2-yl)-3- | | |
| | (hydroxymethyl)-1-methyl-6,7- | | |
| | dihydroimidazo[1,5-α]pyrazin- | | |
| | 8(5H)-one | | |
| 201 | 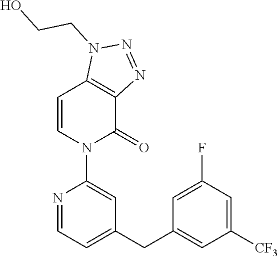 [SMILES: O=c1c2nnn(CCO)c2ccn1-c1cc(Cc2cc(F)cc(C(F)(F)F)c2)ccn1] | 434.1 | ¹H NMR (400 MHz, DMSO-d₆) δ = 8.53 (d, J = 5.2 Hz, 1H), 7.88 (d, J = 7.6 Hz, 1H), 7.77 (s, 1H), 7.64 (s, 1H), 7.57 (br t, J = 8.8 Hz, 2H), 7.45 (dd, J = 1.2, 5.2 Hz, 1H), 6.91 (d, J = 7.6 Hz, 1H), 5.05 (t, J = 5.6 Hz, 1H), 4.65 (t, J = 5.2 Hz, 2H), 4.22 (s, 2H), 3.84 (q, J = 5.6 Hz, 2H). |
| | 5-(4-(3-fluoro-5- | | |
| | (trifluoromethyl)benzyl)pyridin- | | |
| | 2-yl)-1-(2-hydroxyethyl)-1,5- | | |
| | dihydro-4H-[1,2,3 ]triazolo[4,5- | | |
| | c]pyridin-4-one | | |
| 202 | 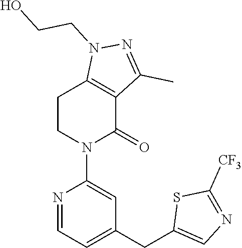 [SMILES: Cc1nn(CCO)c2c1C(=O)N(c1cc(Cc3cnc(C(F)(F)F)s3)ccn1)CC2] | 438.0 | ¹H NMR (400 MHz, DMSO-d₆) δ = 8.36 (d, J = 5.2 Hz, 1H), 8.03 (s, 1H), 7.71 (s, 1H), 7.10 (dd, J = 1.2, 5.2 Hz, 1H), 4.93 (t, J = 5.2 Hz, 1H), 4.39 (s, 2H), 4.21 (t, J = 6.8 Hz, 2H), 4.04 (t, J = 5.2 Hz, 2H), 3.69 (q, J = 5.2 Hz, 2H), 3.04 (t, J = 6.4 Hz, 2H), 2.33 (s, 3H). |
| | 1-(2-hydroxyethyl)-3-methyl-5- | | |
| | (4-((2-(trifluoromethyl)thiazol-5- | | |
| | yl)methyl)pyridin-2-yl)-1,5,6,7- | | |
| | tetrahydro-4H-pyrazolo[4,3- | | |
| | c]pyridin-4-one | | |
| 204 | 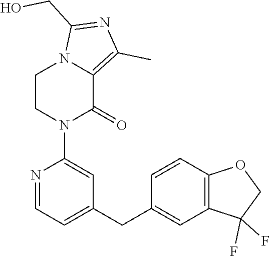 [SMILES: Cc1nc(CO)n2c1C(=O)N(c1cc(Cc3ccc4c(c3)C(F)(F)CO4)ccn1)CC2] | 427.3 | ¹H NMR (400 MHz, DMSO-d₆) δ = 8.33 (d, J = 5.2 Hz, 1H), 7.80 (s, 1H), 7.56 (s, 1H), 7.45 (d, J = 8.4 Hz, 1H), 7.08 (dd, J = 1.2, 5.2 Hz, 1H), 7.04 (d, J = 8.4 Hz, 1H), 5.47 (t, J = 5.6 Hz, 1H), 4.78 (t, J = 16.8 Hz, 2H), 4.51 (d, J = 5.2 Hz, 2H), 4.29 (s, 4H), 4.02 (s, 2H), 2.38 (s, 3H). |
| | 7-(4-((3,3-difluoro-2,3- | | |
| | dihydrobenzofuran-5- | | |
| | yl)methyl)pyridin-2-yl)-3- | | |
| | (hydroxymethyl)-1-methyl-6,7- | | |
| | dihydroimidazo[1,5-α]pyrazin- | | |
| | 8(5H)-one | | |
| 205 | 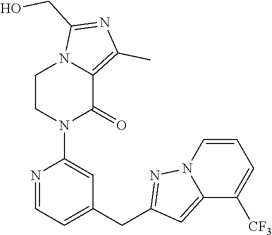 [SMILES: Cc1nc(CO)n2c1C(=O)N(c1cc(Cc3cc4c(C(F)(F)F)cccn4n3)ccn1)CC2] | 457.1 | ¹H NMR (400 MHz, DMSO-d₆) δ = 8.93 (d, J = 7.2 Hz, 1H), 8.35 (d, J = 5.2 Hz, 1H), 7.87 (s, 1H), 7.70 (d, J = 7.2 Hz, 1H), 7.16 (dd, J = 1.2, 5.2 Hz, 1H), 7.06-6.92 (m, 1H), 6.64 (s, 1H), 5.46 (s, 1H), 4.51 (s, 2H), 4.29 (s, 4H), 4.23 (s, 2H), 2.38 (s, 3H). |
| | 3-(hydroxymethyl)-1-methyl-7- | | |
| | (4-((4- | | |
| | (trifluoromethyl)pyrazolo[1,5- | | |
| | α]pyridin-2-yl)methyl)pyridin-2- | | |
| | yl)-6,7-dihydroimidazo[1,5- | | |
| | α]pyrazin-8(5H)-one | | |
| 206 | 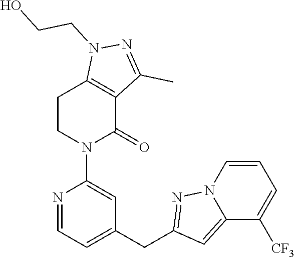 [SMILES: Cc1nn(CCO)c2c1C(=O)N(c1cc(Cc3cc4c(C(F)(F)F)cccn4n3)ccn1)CC2] | 471.3 | ¹H NMR (400 MHz, DMSO-d₆) δ = 8.92 (d, J = 7.2 Hz, 1H), 8.31 (d, J = 5.2 Hz, 1H), 7.82- 7.55 (m, 2H), 7.10 (dd, J = 1.2, 5.2 Hz, 1H), 6.99 (t, J = 7.2 Hz, 1H), 6.63 (s, 1H), 4.93 (s, 1H), 4.22-4.16 (m, 4H), 4.04 (t, J = 5.2 Hz, 2H), 3.72-3.66 (m, 2H), 3.03 (t, J = 6.4 Hz, 2H), 2.33 (s, 3H). |
| | 1-(2-hydroxyethyl)-3-methyl-5- | | |
| | (4-((4- | | |
| | (trifluoromethyl)pyrazolo[1,5- | | |
| | α]pyridin-2-yl)methyl)pyridin-2- | | |
| | yl)-1,5,6,7-tetrahydro-4H- | | |
| | pyrazolo[4,3-c]pyridin-4-one | | |
| 207 | 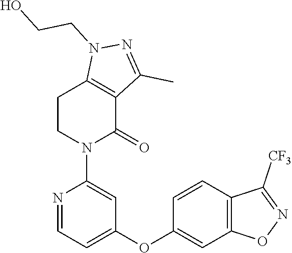 [SMILES: Cc1nn(CCO)c2c1C(=O)N(c1cc(Oc3ccc4c(C(F)(F)F)noc4c3)ccn1)CC2] | 474.0 | ¹H NMR (400 MHz, DMSO-d₆) δ = 8.40 (d, J = 5.6 Hz, 1H), 8.07 (d, J = 8.8 Hz, 1H), 7.90 (d, J = 2.0 Hz, 1H), 7.48 (d, J = 2.4 Hz, 1H), 7.43 (dd, J = 2.0, 8.8 Hz, 1H), 6.93 (dd, J = 2.4, 5.6 Hz, 1H), 4.92 (t, J = 5.2 Hz, 1H), 4.27 (t, J = 6.8 Hz, 2H), 4.04 (t, J = 5.2 Hz, 2H), 3.73- 3.63 (m, 2H), 3.05 (t, J = 6.4 Hz, 2H), 2.29 (s, 3H). |
| | 1-(2-hydroxyethyl)-3-methyl-5- | | |
| | (4-((3- | | |
| | (trifluoromethyl)benzo[d]isoxazo | | |
| | 1-6-yl)oxy)pyridin-2-yl)-1,5,6,7- | | |
| | tetrahydro-4H-pyrazolo[4,3- | | |
| | c]pyridin-4-one | | |
| 208 | 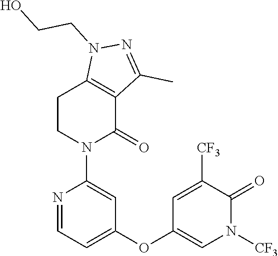 [SMILES: Cc1nn(CCO)c2c1C(=O)N(c1cc(Oc3cc(C(F)(F)F)c(=O)n(C(F)(F)F)c3)ccn1)CC2] | 462.2 | ¹H NMR (400 MHz, DMSO-d₆) δ = 8.31 (d, J = 5.2 Hz, 1H), 8.06 (d, J = 2.0 Hz, 1H), 7.86 (d, J = 2.0 Hz, 1H), 7.63 (s, 1H), 7.09-7.02 (m, 1H), 4.91 (t, J = 5.2 Hz, 1H), 4.19 (t, J = 6.6 Hz, 2H), 4.04 (t, J = 5.2 Hz, 2H), 3.78 (s, 2H), 3.69 (d, J = 5.2 Hz, 2H), 3.47 (s, 3H), 3.03 (t, J = 6.4 Hz, 2H), 2.33 (s, 3H). |
| | 1-(2-hydroxyethyl)-3-methyl-5- | | |
| | (4-((1-methyl-6-oxo-5- | | |
| | (trifluoromethyl)-1,6- | | |
| | dihydropyridin-3- | | |
| | yl)methyl)pyridin-2-yl)-1,5,6,7- | | |
| | tetrahydro-4H-pyrazolo[4,3- | | |
| | c]pyridin-4-one | | |
| 209 | 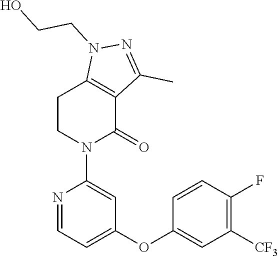 [SMILES: Cc1nn(CCO)c2c1C(=O)N(c1cc(Oc3ccc(F)c(C(F)(F)F)c3)ccn1)CC2] | 451.2 | ¹H NMR (400 MHz, DMSO-d₆) δ = 8.33 (d, J = 5.6 Hz, 1H), 7.69-7.57 (m, 3H), 7.36 (d, J = 2.4 Hz, 1H), 6.79 (dd, J = 2.4, 5.6 Hz, 1H), 4.91 (t, J = 5.6 Hz, 1H), 4.25 (t, J = 6.4 Hz, 2H), 4.04 (t, J = 5.2 Hz, 2H), 3.68 (q, J = 5.2 Hz, 2H), 3.03 (t, J = 6.8 Hz, 2H), 2.31 (s, 3H). |
| | 5-(4-(4-fluoro-3- | | |
| | (trifluoromethyl)phenoxy)pyridin- | | |
| | 2-yl)-1-(2-hydroxyethyl)-3- | | |
| | methyl-1,5,6,7-tetrahydro-4H- | | |
| | pyrazolo[4,3-c]pyridin-4-one | | |
| 211 | 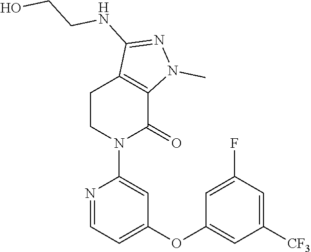 [SMILES: Cn1nc(NCCO)c2c1C(=O)N(c1cc(Oc3cc(F)cc(C(F)(F)F)c3)ccn1)CC2] | 466.0 | ¹H NMR (400 MHz, DMSO-d₆) δ = 8.40 (d, J = 5.6 Hz, 1 H), 7.64 (br d, J = 8.8 Hz, 1 H), 7.53 (br d, J = 9.6 Hz, 1 H), 7.48 (s, 1 H), 7.44 (d, J = 2.4 Hz, 1 H), 6.87-6.96 (m, 1 H), 5.23 (t, J = 6.0 Hz, 1 H), 4.58-4.70 (m, 1 H), 4.20 (t, J = 6.4 Hz, 2 H), 3.85 (s, 3 H), 3.50-3.55 (m, 2 H), 3.14-3.19 (m, 2 H), 2.68 (t, J = 6.4 Hz, 2 H). |
| | 6-(4-(3-fluoro-5- | | |
| | (trifluoromethyl)phenoxy)pyridin- | | |
| | 2-yl)-3-((2- | | |
| | hydroxyethyl)amino)-1-methyl- | | |
| | 1,4,5,6-tetrahydro-7H- | | |
| | pyrazolo[3,4-c]pyridin-7-one | | |
| 212 | 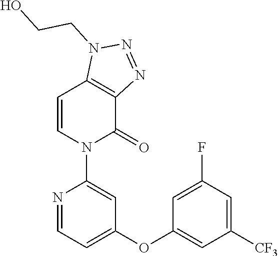 [SMILES: O=c1c2nnn(CCO)c2ccn1-c1cc(Oc2cc(F)cc(C(F)(F)F)c2)ccn1] | 436.1 | ¹H NMR (400 MHz, DMSO-d₆) δ = 8.56 (d, J = 6.0 Hz, 1H), 7.92 (d, J = 7.6 Hz, 1H), 7.71- 7.50 (m, 4H), 7.20 (dd, J = 2.4, 5.6 Hz, 1H), 6.93 (d, J = 7.6 Hz, 1H), 5.05 (t, J = 5.6 Hz, 1H), 4.65 (t, J = 5.2 Hz, 2H), 3.84 (q, J = 5.2 Hz, 2H). |
| | 5-(4-(3-fluoro-5- | | |
| | (trifluoromethyl)phenoxy)pyridin- | | |
| | 2-yl)-1-(2-hydroxyethyl)-1,5- | | |
| | dihydro-4H-[1,2,3 ]triazolo[4,5- | | |
| | c]pyridin-4-one | | |
| 213 | 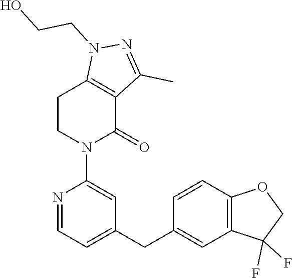 [SMILES: Cc1nn(CCO)c2c1C(=O)N(c1cc(Cc3ccc4c(c3)C(F)(F)CO4)ccn1)CC2] | 441.2 | ¹H NMR (400 MHz, DMSO-d₆) δ = 8.30 (d, J = 5.2 Hz, 1H), 7.62 (s, 1H), 7.55 (s, 1H), 7.44 (d, J = 8.4 Hz, 1H), 7.05-7.00 (m, 2H), 4.94-4.87 (m, 1H), 4.78 (t, J = 16.8 Hz, 2H), 4.19 (t, J = 6.4 Hz, 2H), 4.04 (t, J = 5.2 Hz, 2H), 3.99 (s, 2H), 3.69 (d, J = 4.4 Hz, 2H), 3.03 (t, J = 6.8 Hz, 2H), 2.33 (s, 3H). |
| | 5-(4-((3,3-difluoro-2,3- | | |
| | dihydrobenzofuran-5- | | |
| | yl)methyl)pyridin-2-yl)-1-(2- | | |
| | hydroxyethyl)-3-methyl-1,5,6,7- | | |
| | tetrahydro-4H-pyrazolo[4,3- | | |
| | c]pyridin-4-one | | |
| 214 | 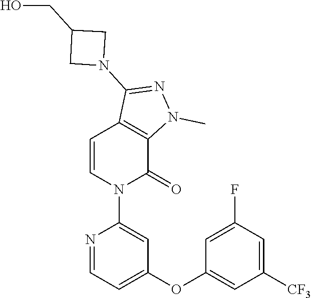 [SMILES: Cn1nc(N2CC(CO)C2)c2ccn(-c3cc(Oc4cc(F)cc(C(F)(F)F)c4)ccn3)c(=O)c21] | 490.1 | ¹H NMR (400 MHz, DMSO-d₆) δ = 8.54 (d, J = 5.6 Hz, 1H), 7.66 (br d, J = 8.4 Hz, 1H), 7.59 (td, J = 2.0, 9.6 Hz, 1H), 7.54 (s, 1H), 7.51 (d, J = 2.0 Hz, 1H), 7.29 (d, J = 7.2 Hz, 1H), 7.16 (dd, J = 2.0, 5.6 Hz, 1H), 6.52 (d, J = 7.2 Hz, 1H), 4.76 (br t, J = 4.8 Hz, 1H), 4.07 (s, 3H), 4.01 (t, J = 7.6 Hz, 2H), 3.75 (t, J = 6.4 Hz, 2H), 3.58 (br t, J = 5.2 Hz, 2H), 2.91-2.77 (m, 1H). |
| | 6-(4-(3-fluoro-5- | | |
| | (trifluoromethyl)phenoxy)pyridin- | | |
| | 2-yl)-3-(3- | | |
| | (hydroxymethyl)azetidin-1-yl)-1- | | |
| | methyl-1,6-dihydro-7H- | | |
| | pyrazolo[3,4-c]pyridin-7-one | | |
| 215 | 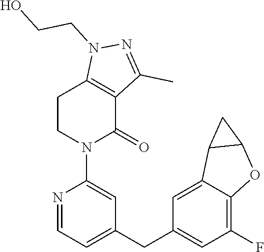 [SMILES: Cc1nn(CCO)c2c1C(=O)N(c1cc(Cc3cc(F)c4c(c3)C3CC3O4)ccn1)CC2] | 435.3 | ¹H NMR (400 MHz, DMSO-d₆) δ = 8.29 (d, J = 5.2 Hz, 1H), 7.60 (s, 1H), 7.10 (d, J = 0.8 Hz, 1H), 7.04-7.00 (m, 1H), 6.95 (d, J = 12.0 Hz, 1H), 5.07-5.01 (m, 1H), 4.92 (t, J = 5.2 Hz, 1H), 4.19 (t, J = 6.4 Hz, 2H), 4.04 (t, J = 5.2 Hz, 2H), 3.89 (s, 2H), 3.69 (q, J = 5.2 Hz, 2H), 3.03 (t, J = 6.4 Hz, 2H), 2.76-2.69 (m, 1H), 2.34 (s, 3H), 1.14-1.06 (m, 1H), 0.37-0.21 (m, 1H). |
| | 5-(4-((3-fluoro-1a,6b-dihydro- | | |
| | 1H-cyclopropa[b]benzofuran-5- | | |
| | yl)methyl)pyridin-2-yl)-1-(2- | | |
| | hydroxyethyl)-3-methyl-1,5,6,7- | | |
| | tetrahydro-4H-pyrazolo[4,3- | | |
| | c]pyridin-4-one | | |
| 216 | 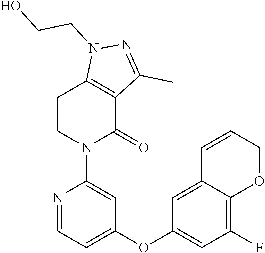 [SMILES: Cc1nn(CCO)c2c1C(=O)N(c1cc(Oc3cc(F)c4c(c3)C=CCO4)ccn1)CC2] | 435.3 | ¹H NMR (400 MHz, DMSO-d₆) δ = 8.29 (d, J = 5.2 Hz, 1H), 7.58 (s, 1H), 7.06-6.93 (m, 2H), 6.79 (s, 1H), 6.49 (dd, J = 2.0, 10.0 Hz, 1H), 6.00-5.90 (m, 1H), 4.92 (br s, 1H), 4.81 (dd, J = 2.0, 3.6 Hz, 2H), 4.19 (t, J = 6.4 Hz, 2H), 4.04 (t, J = 5.2 Hz, 2H), 3.85 (s, 2H), 3.69 (br s, 2H), 3.03 (t, J = 6.4 Hz, 2H), 2.33 (s, 3H). |
| | 5-(4-((8-fluoro-2H-chromen-6- | | |
| | yl)methyl)pyridin-2-yl)-1-(2- | | |
| | hydroxyethyl)-3-methyl-1,5,6,7- | | |
| | tetrahydro-4H-pyrazolo[4,3- | | |
| | c]pyridin-4-one | | |
| 217 | 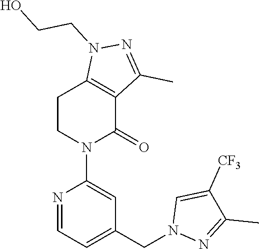 [SMILES: Cc1nn(Cc2ccnc(N3CCc4c(c(C)nn4CCO)C3=O)c2)cc1C(F)(F)F] | 435.3 | ¹H NMR (400 MHz, DMSO-d₆) δ = 8.45 (s, 1H), 8.39-8.34 (m, 2H), 7.86 (s, 1H), 7.64 (s, 1H), 7.54 (s, 1H), 6.91 (d, J = 4.4 Hz, 1H), 6.80 (d, J = 4.8 Hz, 1H), 5.47 (s, 1H), 5.35 (s, 2H), 4.91 (t, J = 5.2 Hz, 2H), 4.21 (t, J = 6.4 Hz, 3H), 4.05 (t, J = 5.2 Hz, 3H), 3.69 (q, J = 5.2 Hz, 3H), 3.04 (t, J = 6.4 Hz, 3H), 2.37- 2.32 (m, 6H), 2.23 (s, 3H). |
| | 1-(2-hydroxyethyl)-3-methyl-5- | | |
| | (4-((3-methyl-4- | | |
| | (trifluoromethyl)-1H-pyrazol-1- | | |
| | yl)methyl)pyridin-2-yl)-1,5,6,7- | | |
| | tetrahydro-4H-pyrazolo[4,3- | | |
| | c]pyridin-4-one | | |
| 218 | 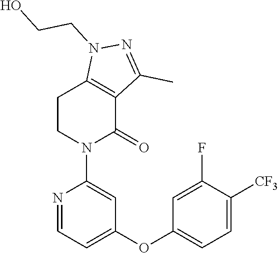 [SMILES: Cc1nn(CCO)c2c1C(=O)N(c1cc(Oc3ccc(C(F)(F)F)c(F)c3)ccn1)CC2] | 451.1 | ¹H NMR (400 MHz, DMSO-d₆) δ = 8.40 (d, J = 6.0 Hz, 1H), 7.87 (t, J = 8.4 Hz, 1H), 7.49 (d, J = 2.4 Hz, 1H), 7.44 (dd, J = 1.6, 11.2 Hz, 1H), 7.17 (d, J = 10.0 Hz, 1H), 6.94 (dd, J = 2.4, 5.6 Hz, 1H), 4.92 (t, J = 5.6 Hz, 1H), 4.28 (t, J = 6.4 Hz, 2H), 4.05 (t, J = 5.2 Hz, 2H), 3.69 (q, J = 5.2 Hz, 2H), 3.05 (t, J = 6.8 Hz, 2H), 2.31 (s, 3H). |
| | 5-(4-(3-fluoro-4- | | |
| | (trifluoromethyl)phenoxy)pyridin- | | |
| | 2-yl)-1-(2-hydroxyethyl)-3- | | |
| | methyl-1,5,6,7-tetrahydro-4H- | | |
| | pyrazolo[4,3-c]pyridin-4-one | | |
| 219 | 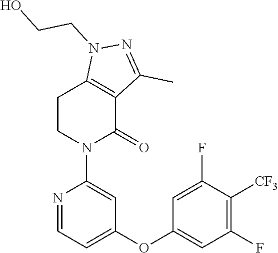 [SMILES: Cc1nn(CCO)c2c1C(=O)N(c1cc(Oc3cc(F)c(C(F)(F)F)c(F)c3)ccn1)CC2] | 469.1 | ¹H NMR (400 MHz, DMSO-d₆) δ = 8.43 (d, J = 5.6 Hz, 1H), 7.55 (d, J = 2.0 Hz, 1H), 7.26 (br d, J = 11.2 Hz, 2H), 6.99 (dd, J = 2.0, 5.6 Hz, 1H), 4.93 (t, J = 5.2 Hz, 1H), 4.28 (br t, J = 6.4 Hz, 2H), 4.05 (br t, J = 5.2 Hz, 2H), 3.69 (q, J = 5.2 Hz, 2H), 3.05 (br t, J = 6.4 Hz, 2H), 2.32 (s, 3H). |
| | 5-(4-(3,5-difluoro-4- | | |
| | (trifluoromethyl)phenoxy)pyridin- | | |
| | 2-yl)-1-(2-hydroxyethyl)-3- | | |
| | methyl-1,5,6,7-tetrahydro-4H- | | |
| | pyrazolo[4,3-c]pyridin-4-one | | |
| 220 | 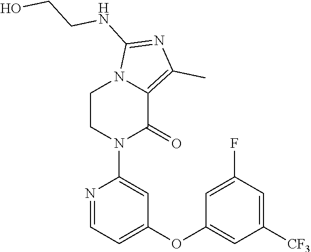 [SMILES: Cc1nc(NCCO)n2c1C(=O)N(c1cc(Oc3cc(F)cc(C(F)(F)F)c3)ccn1)CC2] | 466.2 | ¹H NMR (400 MHz, DMSO-d₆) δ = 8.35 (d, J = 5.6 Hz, 1H), 7.63 (d, J = 8.4 Hz, 1H), 7.58 (d, J = 2.4 Hz, 1H), 7.55-7.49 (m, 1H), 7.46 (s, 1H), 6.86 (dd, J = 2.4, 5.6 Hz, 1H), 6.52 (t, J = 5.6 Hz, 1H), 4.81 (t, J = 5.6 Hz, 1H), 4.32-4.25 (m, 2H), 3.91 (t, J = 5.6 Hz, 2H), 3.52 (q, J = 5.6 Hz, 2H), 3.32-3.28 (m, 2H), 2.28 (s, 3H). |
| | 7-(4-(3-fluoro-5- | | |
| | (trifluoromethyl)phenoxy)pyridin- | | |
| | 2-yl)-3-((2- | | |
| | hydroxyethyl)amino)-1-methyl- | | |
| | 6,7-dihydroimidazo[1,5- | | |
| | α]pyrazin-8(5H)-one | | |
| 221 | 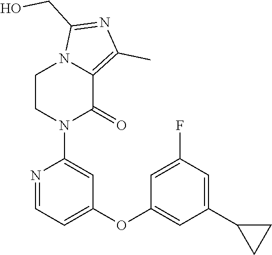 [SMILES: Cc1nc(CO)n2c1C(=O)N(c1cc(Oc3cc(F)cc(C4CC4)c3)ccn1)CC2] | 409.1 | ¹H NMR (400 MHz, DMSO-d₆) δ = 8.34 (d, J = 5.6 Hz, 1H), 7.51 (d, J = 2.0 Hz, 1H), 6.89- 6.73 (m, 4H), 5.47 (t, J = 5.6 Hz, 1H), 4.51 (d, J = 5.6 Hz, 2H), 4.36-4.27 (m, 4H), 2.36 (s, 3H), 2.05-1.90 (m, 1H), 1.02- 0.93 (m, 2H), 0.83-0.73 (m, 2H). |
| | 7-(4-(3-cyclopropyl-5- | | |
| | fluorophenoxy)pyridin-2-yl)-3- | | |
| | (hydroxymethyl)-1-methyl-6,7- | | |
| | dihydroimidazo[1,5-α]pyrazin- | | |
| | 8(5H)-one | | |
| 223 | 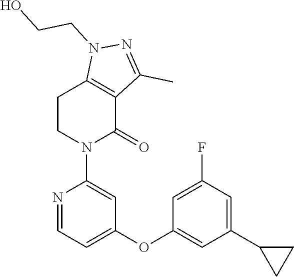 [SMILES: Cc1nn(CCO)c2c1C(=O)N(c1cc(Oc3cc(F)cc(C4CC4)c3)ccn1)CC2] | 423.1 | ¹H NMR (400 MHz, DMSO-d₆) δ = 8.31 (d, J = 5.6 Hz, 1H), 7.32 (d, J = 2.4 Hz, 1H), 6.88-6.82 (m, 2H), 6.80-6.75 (m, 2H), 4.93 (t, J = 5.6 Hz, 1H), 4.24 (t, J = 6.8 Hz, 2H), 4.04 (t, J = 5.2 Hz, 2H), 3.68 (q, J = 5.2 Hz, 2H), 3.03 (t, J = 6.4 Hz, 2H), 2.31 (s, 3H), 2.00-1.92 (m, 1H), 1.00-0.92 (m, 2H), 0.80-0.71 (m, 2H). |
| | 5-(4-(3-cyclopropyl-5- | | |
| | fluorophenoxy)pyridin-2-yl)-1- | | |
| | (2-hydroxyethyl)-3-methyl- | | |
| | 1,5,6,7-tetrahydro-4H- | | |
| | pyrazolo[4,3-c]pyridin-4-one | | |
| 224 | 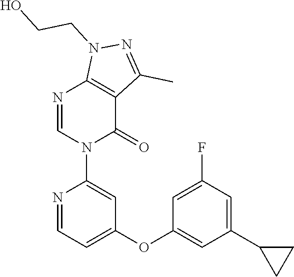 [SMILES: Cc1nn(CCO)c2ncn(-c3cc(Oc4cc(F)cc(C5CC5)c4)ccn3)c(=O)c12] | 422.3 | ¹H NMR (400 MHz, DMSO-d₆) δ = 8.52 (d, J = 5.6 Hz, 1H), 8.46 (s, 1H), 7.34 (d, J = 2.4 Hz, 1H), 7.13 (dd, J = 2.4, 6.0 Hz, 1H), 6.96-6.88 (m, 2H), 6.84 (s, 1H), 4.87 (t, J = 5.6 Hz, 1H), 4.27 (t, J = 6.0 Hz, 2H), 3.79 (q, J = 5.6 Hz, 2H), 2.45 (s, 3H), 2.05-1.94 (m, 1H), 1.04-0.96 (m, 2H), 0.81-0.73 (m, 2H). |
| | 5-(4-(3-cyclopropyl-5- | | |
| | fluorophenoxy)pyridin-2-yl)-1- | | |
| | (2-hydroxyethyl)-3-methyl-1,5- | | |
| | dihydro-4H-pyrazolo[3,4- | | |
| | d]pyrimidin-4-one | | |
| 225 | 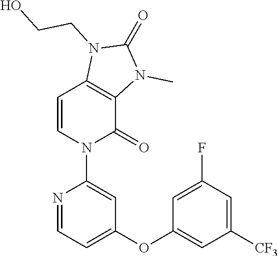 [SMILES: Cn1c(=O)n(CCO)c2ccn(-c3cc(Oc4cc(F)cc(C(F)(F)F)c4)ccn3)c(=O)c21] | 465.1 | ¹H NMR (400 MHz, DMSO-d₆) δ = 8.54 (d, J = 5.6 Hz, 1H), 7.72-7.65 (m, 2H), 7.62-7.57 (m, 1H), 7.56-7.53 (m, 1H), 7.49 (d, J = 2.0 Hz, 1H), 7.19- 7.15 (m, 1H), 6.67 (d, J = 7.6 Hz, 1H), 4.89 (t, J = 5.6 Hz, 1H), 3.85 (t, J = 5.6 Hz, 2H), 3.61 (q, J = 5.6 Hz, 2H), 3.52 (s, 3H). |
| | 5-(4-(3-fluoro-5- | | |
| | (trifluoromethyl)phenoxy)pyridin- | | |
| | 2-yl)-1-(2-hydroxyethyl)-3- | | |
| | methyl-3,5-dihydro-1H- | | |
| | imidazo[4,5-c]pyridine-2,4-dione | | |
| 226 | 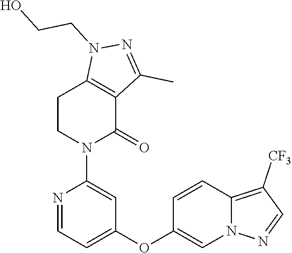 [SMILES: Cc1nn(CCO)c2c1C(=O)N(c1cc(Oc3ccc4c(C(F)(F)F)cnn4c3)ccn1)CC2] | 473.1 | ¹H NMR (400 MHz, DMSO-d₆) δ = 9.16 (s, 1 H), 8.54 (s, 1 H), 8.33 (d, J = 5.6 Hz, 1 H), 7.90 (d, J = 9.2 Hz, 1 H), 7.53 (dd, J = 9.6, 2.0 Hz, 1 H), 7.42 (d, J = 2.4 Hz, 1 H), 6.89 (dd, J = 5.6, 2.4 Hz, 1 H), 4.87-4.95 (m, 1 H), 4.25 (t, J = 6.4 Hz, 2 H), 4.04 (t, J = 5.2 Hz, 2 H), 3.64- 3.70 (m, 2 H), 3.01-3.08 (m, 2 H), 2.29 (s, 3 H). |
| | 1-(2-hydroxyethyl)-3-methyl-5- | | |
| | (4-((3- | | |
| | (trifluoromethyl)pyrazolo[1,5- | | |
| | a]pyridin-6-yl)oxy)pyridin-2-yl)- | | |
| | 1,5,6,7-tetrahydro-4HI- | | |
| | pyrazolo[4,3-c]pyridin-4-one | | |
| 227 | 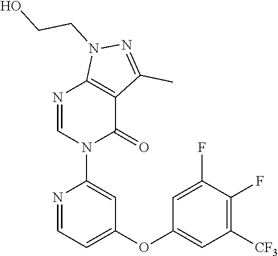 [SMILES: Cc1nn(CCO)c2ncn(-c3cc(Oc4cc(F)c(F)c(C(F)(F)F)c4)ccn3)c(=O)c12] | 468.1 | ¹H NMR (400 MHz, DMSO-d₆) δ = 8.56 (d, J = 5.6 Hz, 1H), 8.44 (s, 1H), 8.01-7.92 (m, 1H), 7.62 (br s, 1H), 7.44 (d, J = 2.4 Hz, 1H), 7.27-7.21 (m, 1H), 4.88 (t, J = 5.6 Hz, 1H), 4.27 (t, J = 5.6 Hz, 2H), 3.79 (q, J = 5.6 Hz, 2H), 2.45 (s, 3H). |
| | 5-(4-(3,4-difluoro-5- | | |
| | (trifluoromethyl)phenoxy)pyridin- | | |
| | 2-yl)-1-(2-hydroxyethyl)-3- | | |
| | methyl-1,5-dihydro-4H- | | |
| | pyrazolo[3,4-d]pyrimidin-4-one | | |
| 228 | 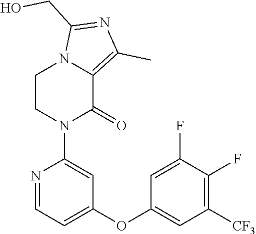 [SMILES: Cc1nc(CO)n2c1C(=O)N(c1cc(Oc3cc(F)c(F)c(C(F)(F)F)c3)ccn1)CC2] | 455.0 | ¹H NMR (400 MHz, DMSO-d₆) δ = 8.38 (d, J = 6.0 Hz, 1H), 7.96-7.86 (m, 1H), 7.59 (d, J = 2.0 Hz, 1H), 7.58-7.53 (m, 1H), 6.89 (dd, J = 2.4, 5.6 Hz, 1H), 5.48 (t, J = 5.6 Hz, 1H), 4.51 (d, J = 5.6 Hz, 2H), 4.38-4.26 (m, 4H), 2.36 (s, 3H). |
| | 7-(4-(3,4-difluoro-5- | | |
| | (trifluoromethyl)phenoxy)pyridin- | | |
| | 2-yl)-3-(hydroxymethyl)-1- | | |
| | methyl-6,7-dihydroimidazo[1,5- | | |
| | α]pyrazin-8(5H)-one | | |
| 229 | 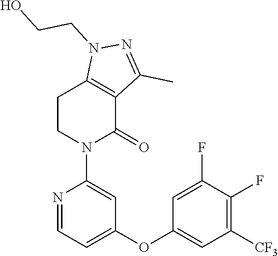 [SMILES: Cc1nn(CCO)c2c1C(=O)N(c1cc(Oc3cc(F)c(F)c(C(F)(F)F)c3)ccn1)CC2] | 469.1 | ¹H NMR (400 MHz, DMSO-d₆) δ = 8.35 (d, J = 5.6 Hz, 1H), 7.94-7.80 (m, 1H), 7.57-7.50 (m, 1H), 7.43 (d, J = 2.4 Hz, 1H), 6.84 (dd, J = 2.4, 6.0 Hz, 1H), 4.92 (t, J = 5.6 Hz, 1H), 4.25 (t, J = 6.4 Hz, 2H), 4.05 (t, J = 5.2 Hz, 2H), 3.69 (q, J = 5.2 Hz, 2H), 3.04 (t, J = 6.4 Hz, 2H), 2.31 (s, 3H). |
| | 5-(4-(3,4-difluoro-5- | | |
| | (trifluoromethyl)phenoxy)pyridin- | | |
| | 2-yl)-1-(2-hydroxyethyl)-3- | | |
| | methyl-1,5,6,7-tetrahydro-4H- | | |
| | pyrazolo[4,3-c]pyridin-4-one | | |
| 230 | 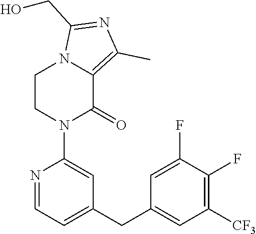 [SMILES: Cc1nc(CO)n2c1C(=O)N(c1cc(Cc3cc(F)c(F)c(C(F)(F)F)c3)ccn1)CC2] | 453.1 | ¹H NMR (400 MHz, DMSO-d₆) δ = 8.35 (d, J = 5.2 Hz, 1H), 7.87-7.78 (m, 2H), 7.63 (d, J = 5.6 Hz, 1H), 7.15-7.11 (m, 1H), 5.46 (t, J = 5.6 Hz, 1H), 4.51 (d, J = 5.6 Hz, 2H), 4.29 (s, 4H), 4.10 (s, 2H), 2.38 (s, 3H). |
| | 7-(4-(3,4-difluoro-5- | | |
| | (trifluoromethyl)benzyl)pyridin- | | |
| | 2-yl)-3-(hydroxymethyl)-1- | | |
| | methyl-6,7-dihydroimidazo[1,5- | | |
| | α]pyrazin-8(5H)-one | | |
| 231 | 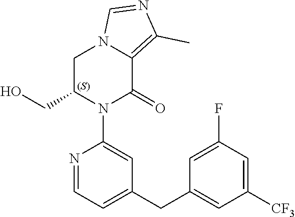 | 435.2 | ¹H NMR (400 MHz, DMSO-d₆) δ = 8.36 (d, J = 4.8 Hz, 1H), 7.87 (s, 1H), 7.79 (s, 1H), 7.59 (s, 1H), 7.53 (t, J = 8.8 Hz, 2H), 7.14 (dd, J = 1.2, 5.2 Hz, 1H), 5.25 (t, J = 5.6 Hz, 1H), 4.92- 4.81 (m, 1H), 4.64-4.53 (m, 1H), 4.25 (dd, J = 4.0, 13.2 Hz, 1H), 4.13 (s, 2H), 3.78-3.71 (m, 1H), 3.15 (m, 1H), 2.40 (s, 3H). |
| | (S)-7-(4-(3-fluoro-5- | | |
| | (trifluoromethyl)benzyl)pyridin- | | |
| | 2-yl)-6-(hydroxymethyl)-1- | | |
| | methyl-6,7-dihydroimidazo[1,5- | | |
| | α]pyrazin-8(5H)-one | | |
| 232 | 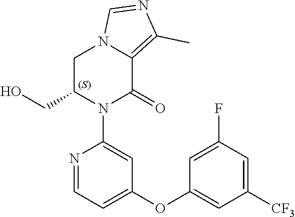 | 437.2 | ¹H NMR (400 MHz, DMSO-d₆) δ = 8.40 (d, J = 5.6 Hz, 1H), 7.81 (br s, 1H), 7.68-7.61 (m, 2H), 7.58-7.51 (m, 1H), 7.48 (s, 1H), 6.92 (dd, J = 2.0, 5.6 Hz, 1H), 5.27 (t, J = 5.6 Hz, 1H), 5.05-4.94 (m, 1H), 4.60 (d, J = 12.8 Hz, 1H), 4.24 (dd, J = 4.0, 13.2 Hz, 1H), 3.78-3.67 (m, 1H), 3.21-3.08 (m, 1H), 2.36 (s, 3H). |
| | (S)-7-(4-(3-fluoro-5- | | |
| | (trifluoromethyl)phenoxy)pyridin- | | |
| | 2-yl)-6-(hydroxymethyl)-1- | | |
| | methyl-6,7-dihydroimidazo[1,5- | | |
| | α]pyrazin-8(5H)-one | | |
| 234 | 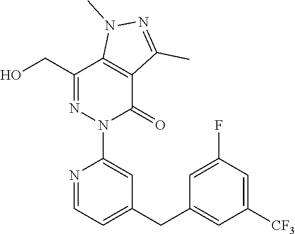 [SMILES: Cc1nn(C)c2c(CO)nn(-c3cc(Cc4cc(F)cc(C(F)(F)F)c4)ccn3)c(=O)c12] | 448.1 | ¹H NMR (400 MHz, CDCl₃) = 8.58 (d, J = 5.2 Hz, 1H), 7.55 (s, 1H), 7.30 (s, 1H), 7.25-7.23 (m, 1H), 7.17-7.07 (m, 2H), 4.95 (s, 2H), 4.26 (s, 3H), 4.12 (s, 2H), 2.66 (s, 3H). |
| | 5-(4-(3-fluoro-5- | | |
| | (trifluoromethyl)benzyl)pyridin- | | |
| | 2-yl)-7-(hydroxymethyl)-1,3- | | |
| | dimethyl-1,5-dihydro-4H- | | |
| | pyrazolo[3,4-d]pyridazin-4-one | | |
| 236 | 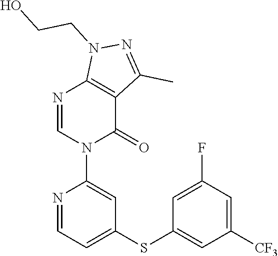 [SMILES: Cc1nn(CCO)c2ncn(-c3cc(Sc4cc(F)cc(C(F)(F)F)c4)ccn3)c(=O)c12] | 466.2 | ¹H NMR (400 MHz, DMSO-d₆) δ = 8.51 (d, J = 5.6 Hz, 1H), 8.45 (s, 1H), 7.89 (t, J = 8.8 Hz, 2H), 7.84 (s, 1H), 7.61 (d, J = 1.2 Hz, 1H), 7.31 (dd, J = 1.6, 5.2 Hz, 1H), 4.88 (t, J = 5.6 Hz, 1H), 4.27 (t, J = 6.0 Hz, 2H), 3.79 (q, J = 5.6 Hz, 2H), 2.44 (s, 3H). |
| | 5-(4-((3-fluoro-5- | | |
| | (trifluoromethyl)phenyl)thio)pyridin- | | |
| | 2-yl)-1-(2-hydroxyethyl)-3- | | |
| | methyl-1,5-dihydro-4H- | | |
| | pyrazolo[3,4-d]pyrimidin-4-one | | |
| 237 | 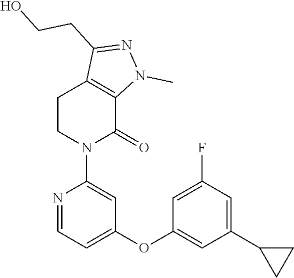 [SMILES: Cn1nc(CCO)c2c1C(=O)N(c1cc(Oc3cc(F)cc(C4CC4)c3)ccn1)CC2] | 423.1 | ¹H NMR (400 MHz, DMSO-d₆) δ = 8.35 (d, J-5.6 Hz, 1 H), 7.35 (d, J =2.4 Hz, 1 H), 6.86 (dd, J =10.0, 1.6 Hz, 2 H), 6.81-6.84 (m, 1 H), 6.77-6.80 (m, 1 H), 4.62-4.75 (m, 1 H), 4.17-4.25 (m, 2 H), 3.99 (s, 3 H), 3.57- 3.64 (m, 2 H), 2.77-2.84 (m, 2 H), 2.66-2.72 (m, 2 H), 1.92- 2.05 (m, 1 H), 0.96-1.03 (m, 2 H), 0.74-0.80 (m, 2 H). |
| | 6-(4-(3-cyclopropyl-5- | | |
| | fluorophenoxy)pyridin-2-yl)-3- | | |
| | (2-hydroxyethyl)-1-methyl- | | |
| | 1,4,5,6-tetrahydro-7H- | | |
| | pyrazolo[3,4-c]pyridin-7-one | | |
| 238 | 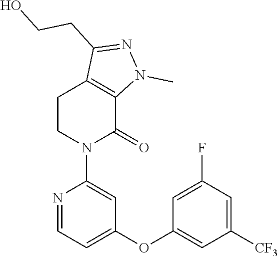 [SMILES: Cn1nc(CCO)c2c1C(=O)N(c1cc(Oc3cc(F)cc(C(F)(F)F)c3)ccn1)CC2] | 451.1 | ¹H NMR (400 MHz, DMSO-d₆) δ = 8.41 (d, J = 5.6 Hz, 1 H), 7.65 (br d, J = 8.4 Hz, 1 H), 7.53-7.59 (m, 1 H), 7.49 (s, 1 H), 7.47 (d, J = 2.4 Hz, 1 H), 6.93 (dd, J = 5.6, 2.4 Hz, 1 H), 4.61-4.71 (m, 1 H), 4.21-4.29 (m, 2 H), 3.99 (s, 3 H), 3.55-3.63 (m, 2 H), 2.82 (t, J = 6.4 Hz, 2 H), 2.67-2.73 (m, 2 H). |
| | 6-(4-(3-fluoro-5- | | |
| | (trifluoromethyl)phenoxy)pyridin- | | |
| | 2-yl)-3-(2-hydroxyethyl)-1- | | |
| | methyl-1,4,5,6-tetrahydro-7H- | | |
| | pyrazolo[3,4-c]pyridin-7-one | | |
| 239 | 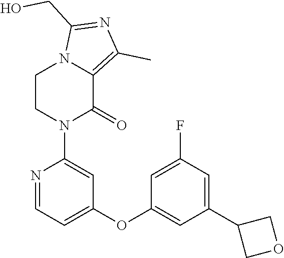 [SMILES: Cc1nc(CO)n2c1C(=O)N(c1cc(Oc3cc(F)cc(C4COC4)c3)ccn1)CC2] | 425.2 | ¹H NMR (400 MHz, DMSO-d₆) δ = 8.36 (d, J = 5.6 Hz, 1H), 7.54 (d, J = 2.0 Hz, 1H), 7.21 (d, J = 9.2 Hz, 1H), 7.11 (s, 1H), 7.04 (d, J = 9.6 Hz, 1H), 6.87 (dd, J = 2.0, 5.6 Hz, 1H), 5.48 (s, 1H), 4.91 (dd, J = 6.0, 8.4 Hz, 2H), 4.64 (t, J = 6.4 Hz, 2H), 4.51 (s, 2H), 4.31 (dd, J = 6.4, 15.6 Hz, 5H), 2.34 (s, 3H). |
| | 7-(4-(3-fluoro-5-(oxetan-3- | | |
| | yl)phenoxy)pyridin-2-yl)-3- | | |
| | (hydroxymethyl)-1-methyl-6,7- | | |
| | dihydroimidazo[1,5-α]pyrazin- | | |
| | 8(5H)-one | | |
| 240 | 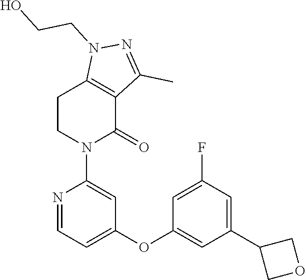 [SMILES: Cc1nn(CCO)c2c1C(=O)N(c1cc(Oc3cc(F)cc(C4COC4)c3)ccn1)CC2] | 439.2 | ¹H NMR (400 MHz, DMSO-d₆) δ = 8.33 (d, J = 5.6 Hz, 1H), 7.37 (d, J = 2.0 Hz, 1H), 7.19 (d, J = 9.6 Hz, 1H), 7.09 (s, 1H), 7.06-6.98 (m, 1H), 6.82 (dd, J = 2.0, 5.6 Hz, 1H), 4.99-4.83 (m, 3H), 4.64 (t, J = 6.4 Hz, 2H), 4.35-4.18 (m, 3H), 4.04 (t, J = 5.2 Hz, 2H), 3.74-3.63 (m, 2H), 3.03 (t, J = 6.4 Hz, 2H), 2.30 (s, 3H). |
| | 5-(4-(3-fluoro-5-(oxetan-3- | | |
| | yl)phenoxy)pyridin-2-yl)-1-(2- | | |
| | hydroxyethyl)-3-methyl-1,5,6,7- | | |
| | tetrahydro-4H-pyrazolo[4,3- | | |
| | c]pyridin-4-one | | |
| 241 | 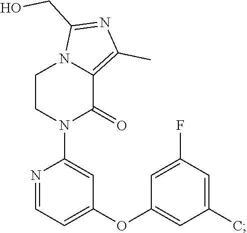 | 403.2 | ¹H NMR (400 MHz, DMSO-d₆) δ = 8.39 (d, J = 6.4 Hz, 1H), 7.59 (d, J = 2.0 Hz, 1H), 7.44- 7.37 (m, 1H), 7.24-7.17 (m, 2H), 6.94-6.87 (m, 1H), 5.47 (t, J = 5.6 Hz, 1H), 4.51 (d, J = 5.6 Hz, 2H), 4.38-4.33 (m, 2H), 4.32-4.26 (m, 2H), 2.36 (s, 3H). |
| | 7-(4-(3-chloro-5- | | |
| | fluorophenoxy)pyridin-2-yl)-3- | | |
| | (hydroxymethyl)-1-methyl-6,7- | | |
| | dihydroimidazo[1,5-α]pyrazin- | | |
| | 8(5H)-one | | |
| 242 | 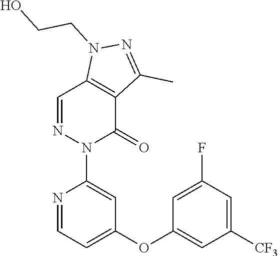 [SMILES: Cc1nn(CCO)c2cnn(-c3cc(Oc4cc(F)cc(C(F)(F)F)c4)ccn3)c(=O)c12] | 450.3 | ¹H NMR (500 MHz, DMSO-d₆) δ = 8.58 (s, 2H), 8.53 (d, J = 5.5 Hz, 2H), 7.70-7.65 (m, 2H), 7.62 (s, 2H), 7.56 (s, 2H), 7.29 (d, J = 2.0 Hz, 2H), 7.17 (dd, J = 2.0, 5.5 Hz, 2H), 4.98 (t, J = 5.5 Hz, 2H), 4.44 (t, J = 5.0 Hz, 4H), 3.76 (q, J = 5.5 Hz, 4H). |
| | 5-(4-(3-fluoro-5- | | |
| | (trifluoromethyl)phenoxy)pyridin- | | |
| | 2-yl)-1-(2-hydroxyethyl)-3- | | |
| | methyl-1,5-dihydro-4H- | | |
| | pyrazolo[3,4-d]pyridazin-4-one | | |
| 243 | 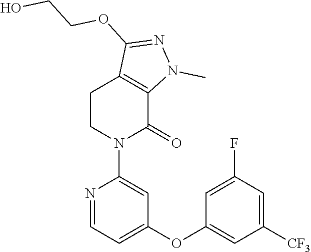 [SMILES: Cn1nc(OCCO)c2c1C(=O)N(c1cc(Oc3cc(F)cc(C(F)(F)F)c3)ccn1)CC2] | 467.1 | ¹H NMR (400 MHz, DMSO-d₆) δ = 8.41 (d, J =5.6 Hz, 1 H), 7.65 (br d, J =8.4 Hz, 1 H), 7.55 (br d, J-9.6 Hz, 1 H), 7.49 (s, 1 H), 7.46 (d, J-2.4 Hz, 1 H), 6.94 (dd, J = 5.6, 2.4 Hz, 1 H), 4.85 (t, J = 5.6 Hz, 1 H), 4.20-4.27 (m, 2 H), 4.11-4.17 (m, 2 H), 3.92 (s, 3 H), 3.64-3.73 (m, 2 H), 2.73 (t, J = 6.4 Hz, 2 H). |
| | 6-(4-(3-fluoro-5- | | |
| | (trifluoromethyl)phenoxy)pyridin- | | |
| | 2-yl)-3-(2-hydroxyethoxy)-1- | | |
| | methyl-1,4,5,6-tetrahydro-7H- | | |
| | pyrazolo[3,4-c]pyridin-7-one | | |
| 244 | 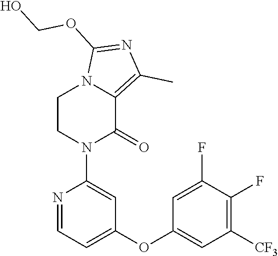 [SMILES: Cc1nc(OCO)n2c1C(=O)N(c1cc(Oc3cc(F)c(F)c(C(F)(F)F)c3)ccn1)CC2] | 469.4 | ¹H NMR (400 MHz, DMSO-d₆) δ = 8.37 (d, J = 5.6 Hz, 1H), 7.95-7.86 (m, 1H), 7.61-7.51 (m, 2H), 6.88 (dd, J = 2.4, 5.6 Hz, 1H), 4.81 (t, J = 5.2 Hz, 1H), 4.37-4.29 (m, 2H), 4.26- 4.18 (m, 2H), 3.75-3.66 (m, 2H), 2.80 (t, J = 6.8 Hz, 2H), 2.36 (s, 3H). |
| | 7-(4-(3,4-difluoro-5- | | |
| | (trifluoromethyl)phenoxy)pyridin- | | |
| | 2-yl)-3-(2-hydroxyethyl)-1- | | |
| | methyl-6,7-dihydroimidazo[1,5- | | |
| | α]pyrazin-8(5H)-one | | |
| 245 | 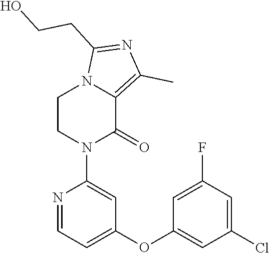 [SMILES: Cc1nc(CCO)n2c1C(=O)N(c1cc(Oc3cc(F)cc(Cl)c3)ccn1)CC2] | 417.2 | ¹H NMR (500 MHz, DMSO-d₆) δ = 8.38 (d, J = 6.0 Hz, 1H), 7.58 (d, J = 2.4 Hz, 1H), 7.39 (td, J = 2.0, 8.8 Hz, 1H), 7.25- 7.15 (m, 2H), 6.89 (dd, J = 2.4, 5.6 Hz, 1H), 4.81 (t, J = 5.2 Hz, 1H), 4.38-4.29 (m, 2H), 4.28- 4.15 (m, 2H), 3.78-3.64 (m, 2H), 2.80 (t, J = 6.4 Hz, 2H), 2.35 (s, 3H). |
| | 7-(4-(3-chloro-5- | | |
| | fluorophenoxy)pyridin-2-yl)-3- | | |
| | (2-hydroxyethyl)-1-methyl-6,7- | | |
| | dihydroimidazo[1,5-α]pyrazin- | | |
| | 8(5H)-one | | |
| 246 | 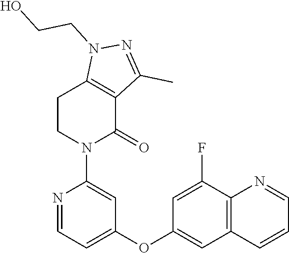 [SMILES: Cc1nn(CCO)c2c1C(=O)N(c1cc(Oc3cc(F)c4ncccc4c3)ccn1)CC2] | 434.2 | ¹H NMR (400 MHz, DMSO-d₆) δ = 8.94 (dd, J = 1.6, 4.0 Hz, 1H), 8.43 (d, J = 8.4 Hz, 1H), 8.37 (d, J = 6.0 Hz, 1H), 7.67 (dd, J = 4.0, 8.4 Hz, 1H), 7.64- 7.60 (m, 2H), 7.47 (d, J = 2.0 Hz, 1H), 6.90 (dd, J = 2.0, 5.6 Hz, 1H), 4.91 (t, J = 5.2 Hz, 1H), 4.27 (t, J = 6.8 Hz, 2H), 4.04 (t, J = 5.2 Hz, 2H), 3.68 (dd, J = 5.2, 10.4 Hz, 2H), 3.04 (t, J = 6.4 Hz, 2H), 2.28 (s, 3H). |
| | 5-(4-((8-fluoroquinolin-6- | | |
| | yl)oxy)pyridin-2-yl)-1-(2- | | |
| | hydroxyethyl)-3-methyl-1,5,6,7- | | |
| | tetrahydro-4H-pyrazolo[4,3- | | |
| | c]pyridin-4-one | | |
| 247 | 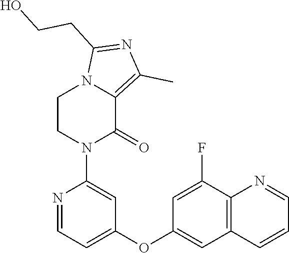 [SMILES: Cc1nc(CCO)n2c1C(=O)N(c1cc(Oc3cc(F)c4ncccc4c3)ccn1)CC2] | 434.2 | ¹H NMR (500 MHz, DMSO-d₆) δ = 8.95 (dd, J = 2.0, 4.5 Hz, 1H), 8.43 (d, J = 8.5 Hz, 1H), 8.38 (d, J = 6.0 Hz, 1H), 7.67 (dd, J = 4.5, 8.5 Hz, 1H), 7.65- 7.60 (m, 3H), 6.94 (dd, J = 2.5, 6.0 Hz, 1H), 4.80 (br s, 1H), 4.34 (dd, J = 5.0, 7.5 Hz, 2H), 4.26-4.18 (m, 2H), 3.74-3.65 (m, 2H), 2.80 (t, J = 6.5 Hz, 2H), 2.32 (s, 3H). |
| | 7-(4-((8-fluoroquinolin-6- | | |
| | yl)oxy)pyridin-2-yl)-3-(2- | | |
| | hydroxyethyl)-1-methyl-6,7- | | |
| | dihydroimidazo[1,5-α]pyrazin- | | |
| | 8(5H)-one | | |
| 248 | 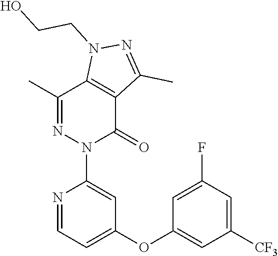 [SMILES: Cc1nn(CCO)c2c(C)nn(-c3cc(Oc4cc(F)cc(C(F)(F)F)c4)ccn3)c(=O)c12] | 464.2 | ¹H NMR (400 MHz, CDCl₃) δ = 8.57 (d, J = 5.6 Hz, 1H), 7.32 (d, J = 2.4 Hz, 1H), 7.25-7.21 (m, 2H), 7.11-7.07 (m, 1H), 6.95 (s, 1H), 4.57-4.54 (m, 2H), 4.09 (t, J = 4.8 Hz, 2H), 2.86 (s, 1H), 2.74 (s, 3H), 2.61 (s, 3H). |
| | 5-(4-(3-fluoro-5- | | |
| | (trifluoromethyl)phenoxy)pyridin- | | |
| | 2-yl)-1-(2-hydroxyethyl)-3,7- | | |
| | dimethyl-1,5-dihydro-4H- | | |
| | pyrazolo[3,4-d]pyridazin-4-one | | |
| 249 | 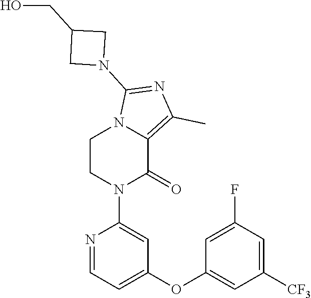 [SMILES: Cc1nc(N2CC(CO)C2)n2c1C(=O)N(c1cc(Oc3cc(F)cc(C(F)(F)F)c3)ccn1)CC2] | 492.2 | ¹H NMR (400 MHz, DMSO-d₆) δ = 8.36 (d, J = 5.6 Hz, 1H), 7.63 (d, J = 8.4 Hz, 1H), 7.57 (d, J = 2.4 Hz, 1H), 7.55-7.50 (m, 1H), 7.47 (s, 1H), 6.89-6.85 (m, 1H), 4.80 (t, J = 5.2 Hz, 1H), 4.30-4.24 (m, 2H), 4.06 (t, J = 8.0 Hz, 2H), 4.02-3.97 (m, 2H), 3.86-3.78 (m, 2H), 3.56 (t, J = 5.6 Hz, 2H), 2.84-2.74 (m, 1H), 2.29 (s, 3H). |
| | 7-(4-(3-fluoro-5- | | |
| | (trifluoromethyl)phenoxy)pyridin- | | |
| | 2-yl)-3-(3- | | |
| | (hydroxymethyl)azetidin-1-yl)-1- | | |
| | methyl-6,7-dihydroimidazo[1,5- | | |
| | α]pyrazin-8(5H)-one | | |
| 251 | 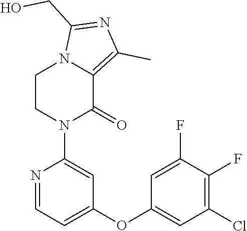 [SMILES: Cc1nc(CO)n2c1C(=O)N(c1cc(Oc3cc(F)c(F)c(Cl)c3)ccn1)CC2] | 421.1 | ¹H NMR (400 MHz, DMSO-d₆) δ = 8.37 (d, J = 6.0 Hz, 1H), 7.60-7.51 (m, 2H), 7.50-7.40 (m, 1H), 6.88 (dd, J = 2.4, 5.6 Hz, 1H), 5.49 (t, J = 5.6 Hz, 1H), 4.52 (d, J = 6.0 Hz, 2H), 4.39-4.25 (m, 4H), 2.37 (s, 3H). |
| | 7-(4-(3-chloro-4,5- | | |
| | difluorophenoxy)pyridin-2-yl)-3- | | |
| | (hydroxymethyl)-1-methyl-6,7- | | |
| | dihydroimidazo[1,5-α]pyrazin- | | |
| | 8(5H)-one | | |
| 252 | 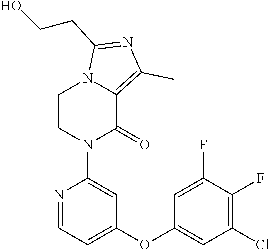 [SMILES: Cc1nc(CCO)n2c1C(=O)N(c1cc(Oc3cc(F)c(F)c(Cl)c3)ccn1)CC2] | 435.0 | ¹H NMR (400 MHz, DMSO-d₆) δ = 8.36 (d, J = 5.6 Hz, 1 H), 7.49-7.60 (m, 2 H), 7.38-7.46 (m, 1 H), 6.86 (dd, J = 5.6, 2.4 Hz, 1 H), 4.77-4.87 (m, 1 H), 4.29-4.43 (m, 2 H), 4.18-4.26 (m, 2 H), 3.62-3.73 (m, 2 H), 2.74-2.86 (m, 2 H), 2.35 (s, 3 H). |
| | 7-(4-(3-chloro-4,5- | | |
| | difluorophenoxy)pyridin-2-yl)-3- | | |
| | (2-hydroxyethyl)-1-methyl-6,7- | | |
| | dihydroimidazo[1,5-α]pyrazin- | | |
| | 8(5H)-one | | |
| 253 | 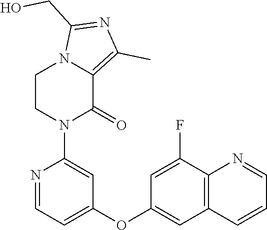 [SMILES: Cc1nc(CO)n2c1C(=O)N(c1cc(Oc3cc(F)c4ncccc4c3)ccn1)CC2] | 420.0 | ¹H NMR (400 MHz, DMSO-d₆) δ = 8.95 (dd, J = 1.6, 4.4 Hz, 1H), 8.45-8.42 (m, 1H), 8.39 (d, J = 5.6 Hz, 1H), 7.69-7.60 (m, 4H), 6.95 (dd, J = 2.4, 5.6 Hz, 1H), 5.47 (t, J = 5.6 Hz, 1H), 4.51 (d, J = 6.0 Hz, 2H), 4.39- 4.27 (m, 4H), 2.33 (s, 3H). |
| | 7-(4-((8-fluoroquinolin-6- | | |
| | yl)oxy)pyridin-2-yl)-3- | | |
| | (hydroxymethyl)-1-methyl-6,7- | | |
| | dihydroimidazo[1,5-α]pyrazin- | | |
| | 8(5H)-one | | |
| 254 | 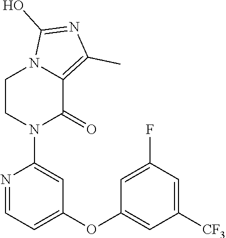 [SMILES: Cc1nc(O)n2c1C(=O)N(c1cc(Oc3cc(F)cc(C(F)(F)F)c3)ccn1)CC2] | 423.1 | ¹H NMR (400 MHz, DMSO-d₆) δ = 10.94 (s, 1H), 8.38 (d, J = 5.6 Hz, 1H), 7.64 (d, J = 8.8 Hz, 1H), 7.54-7.52 (m, 2H), 7.48 (s, 1H), 6.91 (dd, J = 2.0, 5.6 Hz, 1H), 4.25-4.23 (m, 2H), 3.74- 3.72 (m, 2H), 2.28 (s, 3H). |
| | 7-(4-(3-fluoro-5- | | |
| | (trifluoromethyl)phenoxy)pyridin- | | |
| | 2-yl)-3-hydroxy-1-methyl-6,7- | | |
| | dihydroimidazo[1,5-α]pyrazin- | | |
| | 8(5H)-one | | |
| 255 | 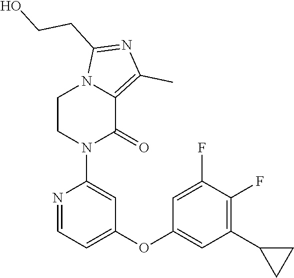 [SMILES: Cc1nc(CCO)n2c1C(=O)N(c1cc(Oc3cc(F)c(F)c(C4CC4)c3)ccn1)CC2] | 441.2 | ¹H NMR (400 MHz, DMSO-d₆) δ = 8.32 (d, J = 6.0 Hz, 1H), 7.45 (d, J = 2.0 Hz, 1H), 7.25- 7.14 (m, 1H), 6.81-6.76 (m, 1H), 6.72-6.67 (m, 1H), 4.85- 4.75 (m, 1H), 4.37-4.26 (m, 2H), 4.25-4.17 (m, 2H), 3.77- 3.63 (m, 2H), 2.80 (t, J = 6.8 Hz, 2H), 2.36 (s, 3H), 2.16-2.07 (m, 1H), 1.07-0.97 (m, 2H), 0.89-0.81 (m, 2H). |
| | 7-(4-(3-cyclopropyl-4,5- | | |
| | difluorophenoxy)pyridin-2-yl)-3- | | |
| | (2-hydroxyethyl)-1-methyl-6,7- | | |
| | dihydroimidazo[1,5-α]pyrazin- | | |
| | 8(5H)-one | | |
| 256 | 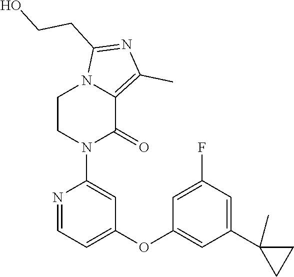 [SMILES: Cc1nc(CCO)n2c1C(=O)N(c1cc(Oc3cc(F)cc(C4(C)CC4)c3)ccn1)CC2] | 437.0 | ¹H NMR (400 MHz, DMSO-d₆) δ = 8.34 (d, J = 5.6 Hz, 1H), 7.50 (d, J = 2.4 Hz, 1H), 6.98- 6.93 (m, 1H), 6.93-6.88 (m, 1H), 6.86 (s, 1H), 6.82 (dd, J = 2.4, 5.6 Hz, 1H), 4.81 (t, J = 5.2 Hz, 1H), 4.35-4.28 (m, 2H), 4.24-4.17 (m, 2H), 3.74-3.66 (m, 2H), 2.80 (t, J = 6.4 Hz, 2H), 2.35 (s, 3H), 1.38 (s, 3H), 0.99-0.91 (m, 2H), 0.84-0.78 (m, 2H). |
| | 7-(4-(3-fluoro-5-(1- | | |
| | methylcyclopropyl)phenoxy)pyridin- | | |
| | 2-yl)-3-(2-hydroxyethyl)-1- | | |
| | methyl-6,7-dihydroimidazo[1,5- | | |
| | α]pyrazin-8(5H)-one | | |
| 257 | 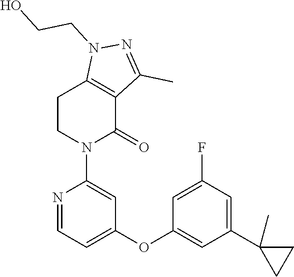 [SMILES: Cc1nn(CCO)c2c1C(=O)N(c1cc(Oc3cc(F)cc(C4(C)CC4)c3)ccn1)CC2] | 437.1 | ¹H NMR (400 MHz, DMSO-d₆) δ = 8.32 (d, J = 5.6 Hz, 1H), 7.33 (d, J = 2.0 Hz, 1H), 6.94 (d, J = 10.4 Hz, 1H), 6.89 (td, J = 2.0, 9.2 Hz, 1H), 6.84 (s, 1H), 6.78 (dd, J = 2.4, 5.6 Hz, 1H), 4.91 (t, J = 5.2 Hz, 1H), 4.24 (t, J = 6.4 Hz, 2H), 4.04 (t, J = 5.2 Hz, 2H), 3.68 (q, J = 5.2 Hz, 2H), 3.03 (t, J = 6.4 Hz, 2H), 2.31 (s, 3H), 1.38 (s, 3H), 0.99- 0.89 (m, 2H), 0.84-0.77 (m, 2H). |
| | 5-(4-(3-fluoro-5-(1- | | |
| | methylcyclopropyl)phenoxy)pyridin- | | |
| | 2-yl)-1-(2-hydroxyethyl)-3- | | |
| | methyl-1,5,6,7-tetrahydro-4H- | | |
| | pyrazolo[4,3-c]pyridin-4-one | | |
| 258 | 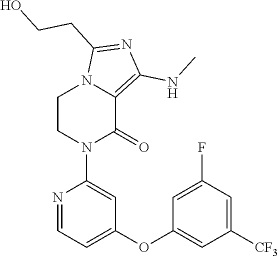 [SMILES: CNc1nc(CCO)n2c1C(=O)N(c1cc(Oc3cc(F)cc(C(F)(F)F)c3)ccn1)CC2] | 466.1 | ¹H NMR (400 MHz, DMSO-d₆) δ = 8.34 (d, J = 5.6 Hz, 1H), 7.64 (br d, J = 2.0 Hz, 2H), 7.56-7.50 (m, 1H), 7.47 (s, 1H), 6.83 (dd, J = 2.0, 5.6 Hz, 1H), 5.82-5.67 (m, 1H), 4.81 (t, J = 5.6 Hz, 1H), 4.35-4.25 (m, 2H), 4.18-4.07 (m, 2H), 3.68 (q, J = 6.0 Hz, 2H), 2.85 (d, J = 4.8 Hz, 3H), 2.73 (t, J = 6.4 Hz, 2H). |
| | 7-(4-(3-fluoro-5- | | |
| | (trifluoromethyl)phenoxy)pyridin- | | |
| | 2-yl)-3-(2-hydroxyethyl)-1- | | |
| | (methylamino)-6,7- | | |
| | dihydroimidazo[1,5-α]pyrazin- | | |
| | 8(5H)-one | | |
| 259 | 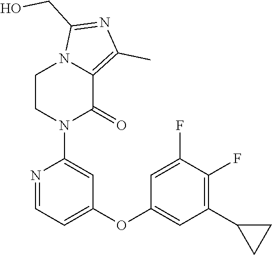 [SMILES: Cc1nc(CO)n2c1C(=O)N(c1cc(Oc3cc(F)c(F)c(C4CC4)c3)ccn1)CC2] | 427.2 | ¹H NMR (400 MHz, DMSO-d₆) δ = 8.33 (d, J = 6.0 Hz, 1H), 7.46 (d, J = 2.4 Hz, 1H), 7.26- 7.15 (m, 1H), 6.82-6.77 (m, 1H), 6.73-6.67 (m, 1H), 5.47 (t, J = 5.6 Hz, 1H), 4.51 (d, J = 6.0 Hz, 2H), 4.36-4.22 (m, 4H), 2.36 (s, 3H), 2.17-2.07 (m, 1H), 1.07-0.99 (m, 2H), 0.89-0.81 (m, 2H). |
| | 7-(4-(3-cyclopropyl-4,5- | | |
| | difluorophenoxy)pyridin-2-yl)-3- | | |
| | (hydroxymethyl)-1-methyl-6,7- | | |
| | dihydroimidazo[1,5-α]pyrazin- | | |
| | 8(5H)-one | | |
| 260 | 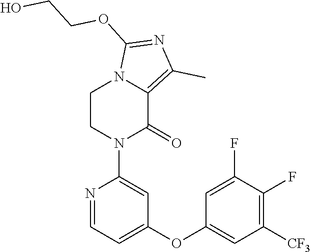 [SMILES: Cc1nc(OCCO)n2c1C(=O)N(c1cc(Oc3cc(F)c(F)c(C(F)(F)F)c3)ccn1)CC2] | 485.2 | ¹H NMR (400 MHz, DMSO-d₆) δ = 8.36 (d, J = 5.6 Hz, 1H), 7.94-7.87 (m, 1H), 7.55 (d, J = 2.4 Hz, 2H), 6.87 (dd, J = 2.4, 5.6 Hz, 1H), 4.94 (t, J = 5.6 Hz, 1H), 4.33 (td, J = 5.2, 16.0 Hz, 4H), 4.00 (t, J = 5.6 Hz, 2H), 3.70 (q, J = 5.2 Hz, 2H), 2.33 (s, 3H). |
| | 7-(4-(3,4-difluoro-5- | | |
| | (trifluoromethyl)phenoxy)pyridin- | | |
| | 2-yl)-3-(2-hydroxyethoxy)-1- | | |
| | methyl-6,7-dihydroimidazo[1,5- | | |
| | α]pyrazin-8(5H)-one | | |
| 261 | 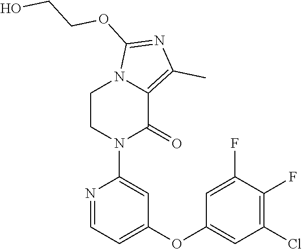 [SMILES: Cc1nc(OCCO)n2c1C(=O)N(c1cc(Oc3cc(F)c(F)c(Cl)c3)ccn1)CC2] | 451.2 | ¹H NMR (400 MHz, CDCl₃) δ = 8.30 (d, J = 5.6 Hz, 1H), 7.67 (d, J = 2.0 Hz, 1H), 7.00-6.95 (m, 1H), 6.94-6.87 (m, 1H), 6.67 (dd, J = 2.0, 5.6 Hz, 1H), 4.59-4.55 (m, 2H), 4.45-4.40 (m, 2H), 4.05-4.01 (m, 2H), 4.01-3.97 (m, 2H), 2.49 (s, 3H). |
| | 7-(4-(3-chloro-4,5- | | |
| | difluorophenoxy)pyridin-2-yl)-3- | | |
| | (2-hydroxyethoxy)-1-methyl- | | |
| | 6,7-dihydroimidazo[1,5- | | |
| | α]pyrazin-8(5H)-one | | |
| 262 | 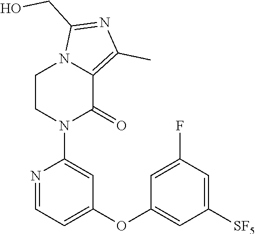 | 495.0 | ¹HNMR (400 MHz, DMSO-d₆) δ = 8.41 (d, J = 5.6 Hz, 1H), 7.96-7.85 (m, 1H), 7.69 (s, 1H), 7.63 (d, J = 2.0 Hz, 1H), 7.58 (br d, J = 9.6 Hz, 1H), 6.93 (dd, J = 2.0, 5.6 Hz, 1H), 5.48 (br t, J = 5.2 Hz, 1H), 4.51 (d, J = 4.8 Hz, 2H), 4.41-4.26 (m, 4H), 2.36 (s, 3H). |
| | 7-(4-(3-fluoro-5-(pentafluoro-16- | | |
| | sulfaneyl)phenoxy)pyridin-2-yl)- | | |
| | 3-(hydroxymethyl)-1-methyl- | | |
| | 6,7-dihydroimidazo[1,5- | | |
| | α]pyrazin-8(5H)-one | | |
| 263 | 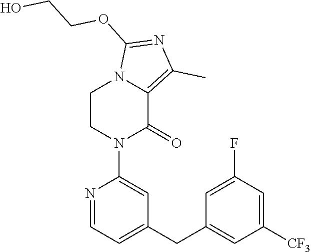 [SMILES: Cc1nc(OCCO)n2c1C(=O)N(c1cc(Cc3cc(F)cc(C(F)(F)F)c3)ccn1)CC2] | 465.2 | ¹H NMR (400 MHz, DMSO-d₆) δ = 8.33 (d, J = 4.8 Hz, 1H), 7.80 (s, 1H), 7.58 (s, 1H), 7.53 (t, J = 8.4 Hz, 2H), 7.10 (d, J = 4.8 Hz, 1H), 4.95 (t, J = 5.6 Hz, 1H), 4.35 (t, J = 4.4 Hz, 2H), 4.25 (t, J = 5.2 Hz, 2H), 4.12 (s, 2H), 4.04-3.95 (m, 2H), 3.77- 3.65 (m, 2H), 2.35 (s, 3H). |
| | 7-(4-(3-fluoro-5- | | |
| | (trifluoromethyl)benzyl)pyridin- | | |
| | 2-yl)-3-(2-hydroxyethoxy)-1- | | |
| | methyl-6,7-dihydroimidazo[1,5- | | |
| | α]pyrazin-8(5H)-one | | |
| 264 | 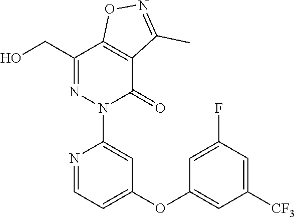 [SMILES: Cc1noc2c(CO)nn(-c3cc(Oc4cc(F)cc(C(F)(F)F)c4)ccn3)c(=O)c12] | 437.0 | ¹H NMR (400 MHz, DMSO-d₆) δ = 8.55 (d, J = 6.0 Hz, 1H), 7.71-7.64 (m, 2H), 7.60 (s, 1H), 7.31 (d, J = 2.4 Hz, 1H), 7.23 (dd, J = 2.4, 5.6 Hz, 1H), 5.82 (t, J = 6.0 Hz, 1H), 4.67 (d, J = 5.6 Hz, 2H), 2.58 (s, 3H). |
| | 5-(4-(3-fluoro-5- | | |
| | (trifluoromethyl)phenoxy)pyridin- | | |
| | 2-yl)-7-(hydroxymethyl)-3- | | |
| | methylisoxazolo[4,5- | | |
| | d]pyridazin-4(5H)-one | | |
| 265 | 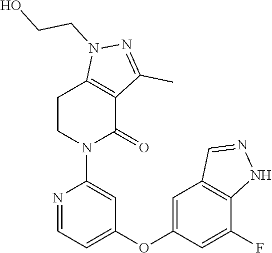 [SMILES: Cc1nn(CCO)c2c1C(=O)N(c1cc(Oc3cc(F)c4[nH]ncc4c3)ccn1)CC2] | 423.1 | ¹H NMR (400 MHz, DMSO-d₆) δ = 14.28-13.28 (m, 1H), 8.29 (d, J = 6.0 Hz, 1H), 8.21 (d, J = 3.2 Hz, 1H), 7.45 (d, J = 1.6 Hz, 1H), 7.31 (d, J = 2.4 Hz, 1H), 7.20 (dd, J = 1.6, 11.2 Hz, 1H), 6.75 (dd, J = 2.4, 6.0 Hz, 1H), 4.97-4.80 (m, 1H), 4.24 (t, J = 6.8 Hz, 2H), 4.03 (t, J = 5.2 Hz, 2H), 3.68 (br s, 2H), 3.02 (t, J = 6.4 Hz, 2H), 2.28 (s, 3H). |
| | 5-(4-((7-fluoro-1H-indazol-5- | | |
| | yl)oxy)pyridin-2-yl)-1-(2- | | |
| | hydroxyethyl)-3-methyl-1,5,6,7- | | |
| | tetrahydro-4H-pyrazolo[4,3- | | |
| | c]pyridin-4-one | | |
| 266 | 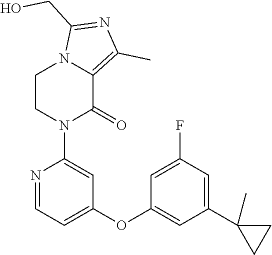 [SMILES: Cc1nc(CO)n2c1C(=O)N(c1cc(Oc3cc(F)cc(C4(C)CC4)c3)ccn1)CC2] | 423.1 | ¹H NMR (400 MHz, DMSO-d₆) δ = 8.49 (d, J = 5.2 Hz, 1H), 7.65 (s, 1H), 7.15-7.02 (m, 2H), 7.01-6.93 (m, 2H), 5.70-5.58 (m, 1H), 4.66 (d, J = 5.2 Hz, 2H), 4.45 (d, J = 8.4 Hz, 4H), 2.65 (s, 3H), 1.53 (s, 3H), 1.16- 0.87 (m, 4H). |
| | 7-(4-(3-fluoro-5-(1- | | |
| | methylcyclopropyl)phenoxy)pyridin- | | |
| | 2-yl)-3-(hydroxymethyl)-1- | | |
| | methyl-6,7-dihydroimidazo[1,5- | | |
| | α]pyrazin-8(5H)-one | | |
| 267 | 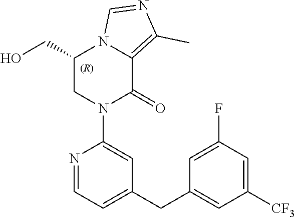 | 435.1 | ¹H NMR (400 MHz, DMSO-d₆) δ = 8.35 (d, J = 5.0 Hz, 1H), 7.92-7.75 (m, 2H), 7.60 (s, 1H), 7.54 (br t, J = 8.0 Hz, 2H), 7.13 (dd, J = 1.2, 5.2 Hz, 1H), 5.27 (t, J = 5.4 Hz, 1H), 4.49 (quin, J = 5.4 Hz, 1H), 4.39-4.23 (m, 2H), 4.14 (s, 2H), 3.81-3.60 (m, 2H), 2.40 (s, 3H). |
| | (R)-7-(4-(3-fluoro-5- | | |
| | (trifluoromethyl)benzyl)pyridin- | | |
| | 2-yl)-5-(hydroxymethyl)-1- | | |
| | methyl-6,7-dihydroimidazo[1,5- | | |
| | α]pyrazin-8(5H)-one | | |
| 268 | 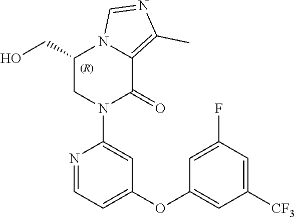 | 437.0 | ¹H NMR (400 MHz, DMSO-d₆) δ = 8.40 (d, J = 5.6 Hz, 1H), 7.83 (s, 1H), 7.65 (br d, J = 8.4 Hz, 1H), 7.61 (d, J = 2.4 Hz, 1H), 7.55 (td, J = 2.0, 9.6 Hz, 1H), 7.50 (s, 1H), 6.91 (dd, J = 2.4, 5.6 Hz, 1H), 5.27 (t, J = 5.6 Hz, 1H), 4.53-4.47 (m, 1H), 4.47-4.39 (m, 1H), 4.36-4.25 (m, 1H), 3.80-3.61 (m, 2H), 2.38 (s, 3H). |
| | (R)-7-(4-(3-fluoro-5- | | |
| | (trifluoromethyl)phenoxy)pyridin- | | |
| | 2-yl)-5-(hydroxymethyl)-1- | | |
| | methyl-6,7-dihydroimidazo[1,5- | | |
| | α]pyrazin-8(5H)-one | | |
| 269 | 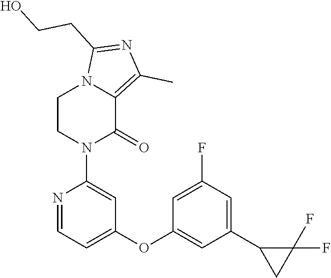 [SMILES: Cc1nc(CCO)n2c1C(=O)N(c1cc(Oc3cc(F)cc(C4CC4(F)F)c3)ccn1)CC2] | 459.1 | ¹H NMR (400 MHz, DMSO-d₆) δ = 8.36 (d, J = 5.6 Hz, 1H), 7.53 (d, J = 2.4 Hz, 1H), 7.11 (d, J = 9.6 Hz, 1H), 7.07 (td, J = 2.4, 9.6 Hz, 1H), 7.03 (s, 1H), 6.83 (dd, J = 2.4, 5.6 Hz, 1H), 4.82 (br s, 1H), 4.37-4.29 (m, 2H), 4.26-4.20 (m, 2H), 3.75- 3.67 (m, 2H), 3.10 (dt, J = 8.4, 12.4 Hz, 1H), 2.80 (t, J = 6.4 Hz, 2H), 2.35 (s, 3H), 2.18-1.97 (m, 2H). |
| | 7-(4-(3-(2,2- | | |
| | difluorocyclopropyl)-5- | | |
| | fluorophenoxy)pyridin-2-yl)-3- | | |
| | (2-hydroxyethyl)-1-methyl-6,7- | | |
| | dihydroimidazo[1,5-α]pyrazin- | | |
| | 8(5H)-one | | |
| 270 | 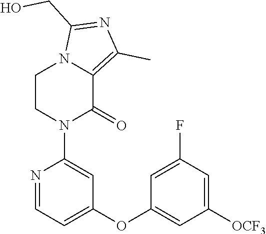 [SMILES: Cc1nc(CO)n2c1C(=O)N(c1cc(Oc3cc(F)cc(OC(F)(F)F)c3)ccn1)CC2] | 453.1 | ¹H NMR (400 MHz, DMSO-d₆) δ = 8.40 (d, J = 6.0 Hz, 1H), 7.62 (d, J = 2.4 Hz, 1H), 7.34 (br d, J = 9.6 Hz, 1H), 7.26 (td, J = 2.0, 9.6 Hz, 1H), 7.17 (s, 1H), 6.95-6.88 (m, 1H), 5.48 (t, J = 5.6 Hz, 1H), 4.51 (d, J = 5.6 Hz, 2H), 4.39-4.26 (m, 4H), 2.36 (s, 3H). |
| | 7-(4-(3-fluoro-5- | | |
| | (trifluoromethoxy)phenoxy)pyridin- | | |
| | 2-yl)-3-(hydroxymethyl)-1- | | |
| | methyl-6,7-dihydroimidazo[1,5- | | |
| | α]pyrazin-8(5H)-one | | |
| 271 | 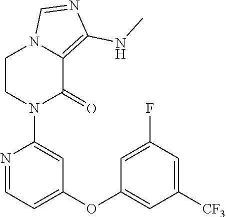 [SMILES: CNc1ncn2c1C(=O)N(c1cc(Oc3cc(F)cc(C(F)(F)F)c3)ccn1)CC2] | 421.9 | ¹H NMR (400 MHz, DMSO-d₆) δ = 8.35 (d, J = 5.6 Hz, 1H), 7.69-7.59 (m, 2H), 7.56-7.49 (m, 2H), 7.46 (s, 1H), 6.85 (dd, J = 2.4, 5.6 Hz, 1H), 5.73 (q, J = 4.8 Hz, 1H), 4.36-4.28 (m, 2H), 4.23-4.15 (m, 2H), 2.85 (d, J = 4.8 Hz, 3H). |
| | 7-(4-(3-fluoro-5- | | |
| | (trifluoromethyl)phenoxy)pyridin- | | |
| | 2-yl)-1-(methylamino)-6,7- | | |
| | dihydroimidazo[1,5-α]pyrazin- | | |
| | 8(5H)-one | | |
| 272 | 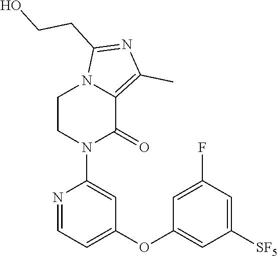 | 509.0 | ¹H NMR (400 MHz, DMSO-d₆) δ = 8.40 (d, J = 5.6 Hz, 1H), 7.89 (br d, J = 8.8 Hz, 1H), 7.69 (s, 1H), 7.62 (d, J = 2.0 Hz, 1H), 7.58 (br d, J = 9.6 Hz, 1H), 6.92 (dd, J = 2.0, 5.6 Hz, 1H), 4.82 (br s, 1H), 4.42-4.31 (m, 2H), 4.26-4.13 (m, 2H), 3.71 (br d, J = 4.4 Hz, 2H), 2.80 (br t, J = 6.0 Hz, 2H), 2.35 (s, 3H). |
| | 7-(4-(3-fluoro-5-(pentafluoro-16- | | |
| | sulfaneyl)phenoxy)pyridin-2-yl)- | | |
| | 3-(2-hydroxyethyl)-1-methyl- | | |
| | 6,7-dihydroimidazo[1,5- | | |
| | α]pyrazin-8(5H)-one | | |
| 278 | 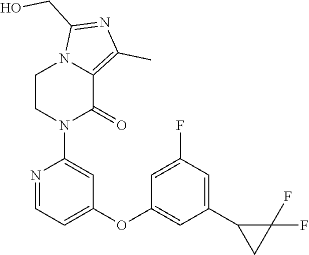 [SMILES: Cc1nc(CO)n2c1C(=O)N(c1cc(Oc3cc(F)cc(C4CC4(F)F)c3)ccn1)CC2] | 445.0 | ¹H NMR (400 MHz, DMSO-d₆) δ = 8.41 (d, J = 5.6 Hz, 1H), 7.59 (d, J = 2.0 Hz, 1H), 7.15 (d, J = 9.6 Hz, 1H), 7.11 (td, J = 2.0, 9.6 Hz, 1H), 7.07 (s, 1H), 6.88 (dd, J = 2.4, 5.6 Hz, 1H), 5.54 (t, J = 5.6 Hz, 1H), 4.56 (d, J = 5.6 Hz, 2H), 4.36 (dd, J = 4.8, 15.2 Hz, 4H), 3.14 (dt, J = 8.4, 12.4 Hz, 1H), 2.40 (s, 3H), 2.24-2.02 (m, 2H). |
| | 7-(4-(3-(2,2- | | |
| | difluorocyclopropyl)-5- | | |
| | fluorophenoxy)pyridin-2-yl)-3- | | |
| | (hydroxymethyl)-1-methyl-6,7- | | |
| | dihydroimidazo[1,5-α]pyrazin- | | |
| | 8(5H)-one | | |
| 279 | 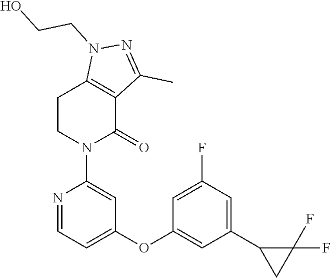 [SMILES: Cc1nn(CCO)c2c1C(=O)N(c1cc(Oc3cc(F)cc(C4CC4(F)F)c3)ccn1)CC2] | 459.2 | ¹H NMR (400 MHz, DMSO-d₆) δ = 8.33 (d, J = 5.6 Hz, 1H), 7.37 (d, J = 2.0 Hz, 1H), 7.09 (d, J = 9.6 Hz, 1H), 7.04 (td, J = 2.0, 9.6 Hz, 1H), 7.01 (s, 1H), 6.79 (dd, J = 2.0, 5.6 Hz, 1H), 4.93 (t, J = 5.6 Hz, 1H), 4.25 (t, J = 6.4 Hz, 2H), 4.04 (t, J = 5.2 Hz, 2H), 3.69 (q, J = 5.2 Hz, 2H), 3.15-3.06 (m, 1H), 3.06- 3.00 (m, 2H), 2.31 (s, 3H), 2.19-1.98 (m, 2H). |
| | 5-(4-(3-(2,2- | | |
| | difluorocyclopropyl)-5- | | |
| | fluorophenoxy)pyridin-2-yl)-1- | | |
| | (2-hydroxyethyl)-3-methyl- | | |
| | 1,5,6,7-tetrahydro-4H- | | |
| | pyrazolo[4,3-c]pyridin-4-one | | |
| 280 | 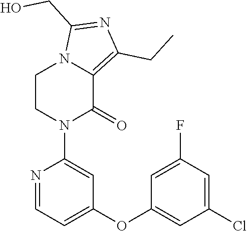 [SMILES: CCc1nc(CO)n2c1C(=O)N(c1cc(Oc3cc(F)cc(Cl)c3)ccn1)CC2] | 417.2 | ¹H NMR (500 MHz, DMSO-d₆) δ = 8.40 (d, J = 6.0 Hz, 1H), 7.60 (d, J = 2.5 Hz, 1H), 7.40 (td, J = 2.0, 9.0 Hz, 1H), 7.25- 7.18 (m, 2H), 6.91 (dd, J = 2.5, 5.5 Hz, 1H), 5.48 (t, J = 5.5 Hz, 1H), 4.54 (d, J = 6.0 Hz, 2H), 4.37-4.31 (m, 4H), 2.81 (q, J = 7.5 Hz, 2H), 1.15 (t, J = 8.0 Hz, 3H). |
| | 7-(4-(3-chloro-5- | | |
| | fluorophenoxy)pyridin-2-yl)-1- | | |
| | ethyl-3-(hydroxymethyl)-6,7- | | |
| | dihydroimidazo[1,5-α]pyrazin- | | |
| | 8(5H)-one | | |
| 281 | 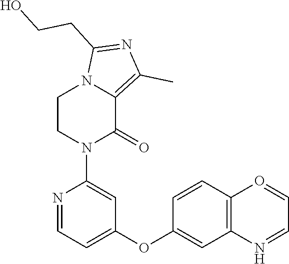 | 422.3 | ¹H NMR (400 MHz, DMSO-d₆) δ = 8.27 (d, J = 5.6 Hz, 1H), 7.49-7.29 (m, 1H), 6.80-6.61 (m, 2H), 6.33 (d, J = 2.4 Hz, 1H), 6.23 (br dd, J = 2.4, 8.8 Hz, 1H), 6.06 (br s, 1H), 4.81 (br t, J = 5.2 Hz, 1H), 4.28 (br d, J = 5.6 Hz, 2H), 4.24-4.17 (m, 2H), 4.12 (br s, 2H), 3.70 (q, J = 6.0 Hz, 2H), 3.32-3.26 (m, 2H), 2.79 (br t, J = 6.4 Hz, 2H), 2.35 (s, 3H). |
| | 7-(4-((3,4-dihydro-2H- | | |
| | benzo[b][1,4]oxazin-6- | | |
| | yl)oxy)pyridin-2-yl)-3-(2- | | |
| | hydroxyethyl)-1-methyl-6,7- | | |
| | dihydroimidazo[1,5-α]pyrazin- | | |
| | 8(5H)-one | | |
| 282 | 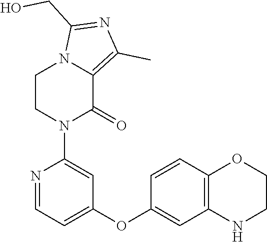 [SMILES: Cc1nc(CO)n2c1C(=O)N(c1cc(Oc3ccc4c(c3)NCCO4)ccn1)CC2] | 408.1 | ¹H NMR (400 MHz, DMSO-d₆) δ = 8.27 (d, J = 5.6 Hz, 1H), 7.41 (d, J = 2.4 Hz, 1H), 6.73 (dd, J = 2.4, 5.6 Hz, 1H), 6.70 (d, J = 8.4 Hz, 1H), 6.34 (d, J = 2.8 Hz, 1H), 6.23 (dd, J = 2.8, 8.4 Hz, 1H), 6.05 (s, 1H), 5.45 (t, J = 5.6 Hz, 1H), 4.51 (d, J = 5.2 Hz, 2H), 4.33-4.26 (m, 4H), 4.12 (t, J = 4.4 Hz, 2H), 3.31- 3.28 (m, 2H), 2.36 (s, 3H). |
| | 7-(4-((3,4-dihydro-2H- | | |
| | benzo[b][1,4]oxazin-6- | | |
| | yl)oxy)pyridin-2-yl)-3- | | |
| | (hydroxymethyl)-1-methyl-6,7- | | |
| | dihydroimidazo[1,5-α]pyrazin- | | |
| | 8(5H)-one | | |
| 283 | 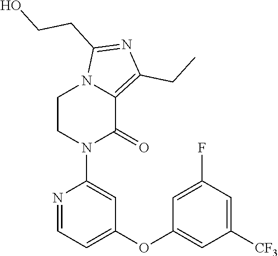 [SMILES: CCc1nc(CCO)n2c1C(=O)N(c1cc(Oc3cc(F)cc(C(F)(F)F)c3)ccn1)CC2] | 465.2 | ¹H NMR (500 MHz, DMSO-d₆) δ = 8.40 (d, J = 5.5 Hz, 1H), 7.64 (d, J = 8.5 Hz, 1H), 7.62 (d, J = 2.5 Hz, 1H), 7.54 (td, J = 2.0, 9.5 Hz, 1H), 7.48 (s, 1H), 6.91 (dd, J = 2.5, 5.5 Hz, 1H), 4.81 (t, J = 5.5 Hz, 1H), 4.36- 4.32 (m, 2H), 4.26-4.21 (m, 2H), 3.74-3.66 (m, 2H), 2.84- 2.76 (m, 4H), 1.14 (t, J = 7.5 Hz, 3H). |
| | 1-ethyl-7-(4-(3-fluoro-5- | | |
| | (trifluoromethyl)phenoxy)pyridin- | | |
| | 2-yl)-3-(2-hydroxyethyl)-6,7- | | |
| | dihydroimidazo[1,5-α]pyrazin- | | |
| | 8(5H)-one | | |
| 285 | 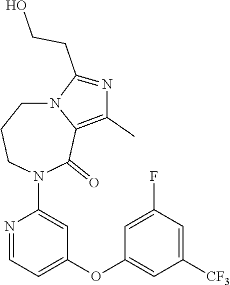 [SMILES: Cc1nc(CCO)n2c1C(=O)N(c1cc(Oc3cc(F)cc(C(F)(F)F)c3)ccn1)CCC2] | 465.3 | ¹H NMR (400 MHz, DMSO-d₆) δ = 8.37 (d, J = 6.0 Hz, 1H), 7.74 (d, J = 2.0 Hz, 1H), 7.65 (br d, J = 8.4 Hz, 1H), 7.55 (br d, J = 9.6 Hz, 1H), 7.50 (s, 1H), 6.88 (dd, J = 2.0, 5.6 Hz, 1H), 4.86-4.77 (m, 1H), 4.16-4.09 (m, 2H), 4.06 (br t, J = 6.0 Hz, 2H), 3.68 (q, J = 6.0 Hz, 2H), 2.81 (t, J = 6.4 Hz, 2H), 2.22 (s, 3H), 2.12-2.03 (m, 2H). |
| | 8-(4-(3-fluoro-5- | | |
| | (trifluoromethyl)phenoxy)pyridin- | | |
| | 2-yl)-3-(2-hydroxyethyl)-1- | | |
| | methyl-5,6,7,8-tetrahydro-9HI- | | |
| | imidazo[1,5-a][1,4]diazepin-9- | | |
| | one | | |
| 286 | 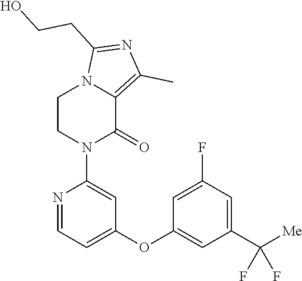 [SMILES: Cc1nc(CCO)n2c1C(=O)N(c1cc(Oc3cc(F)cc(C(C)(F)F)c3)ccn1)CC2] | 447.1 | ¹H NMR (400 MHz, DMSO-d₆) δ = 8.38 (d, J = 5.6 Hz, 1H), 7.57 (d, J = 2.4 Hz, 1H), 7.39- 7.36 (m, 2H), 7.26 (s, 1H), 6.89 (dd, J = 2.0, 5.6 Hz, 1H), 4.81 (t, J = 5.2 Hz, 1H), 4.38-4.29 (m, 2H), 4.26-4.18 (m, 2H), 3.75-3.65 (m, 2H), 2.80 (t, J = 6.8 Hz, 2H), 2.34 (s, 3H), 2.00 (t, J = 19.2 Hz, 3H). |
| | 7-(4-(3-(1,1-difluoroethyl)-5- | | |
| | fluorophenoxy)pyridin-2-yl)-3- | | |
| | (2-hydroxyethyl)-1-methyl-6,7- | | |
| | dihydroimidazo[1,5-α]pyrazin- | | |
| | 8(5H)-one | | |
| 287 | 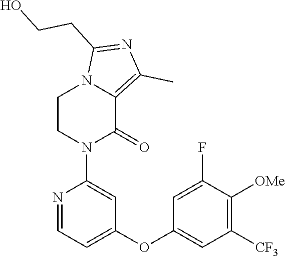 [SMILES: COc1c(F)cc(Oc2ccnc(N3CCn4c(CCO)nc(C)c4C3=O)c2)cc1C(F)(F)F] | 481.1 | ¹H NMR (400 MHz, DMSO-d₆) δ = 8.36 (d, J = 5.6 Hz, 1H), 7.70 (dd, J = 2.8, 12.0 Hz, 1H), 7.56 (d, J = 2.4 Hz, 1H), 7.39- 7.35 (m, 1H), 6.85 (dd, J = 2.4, 5.6 Hz, 1H), 4.80 (t, J = 5.4 Hz, 1H), 4.36-4.29 (m, 2H), 4.26- 4.19 (m, 2H), 3.96 (d, J = 1.6 Hz, 3H), 3.75-3.63 (m, 2H), 2.80 (t, J = 6.6 Hz, 2H), 2.35 (s, 3H). |
| | 7-(4-(3-fluoro-4-methoxy-5- | | |
| | (trifluoromethyl)phenoxy)pyridin- | | |
| | 2-yl)-3-(2-hydroxyethyl)-1- | | |
| | methyl-6,7-dihydroimidazo[1,5- | | |
| | α]pyrazin-8(5H)-one | | |
| 288 | 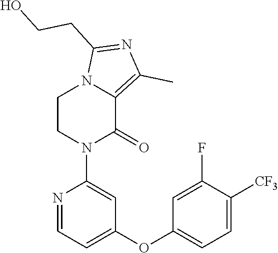 [SMILES: Cc1nc(CCO)n2c1C(=O)N(c1cc(Oc3ccc(C(F)(F)F)c(F)c3)ccn1)CC2] | 451.1 | ¹H NMR (400 MHz, DMSO-d₆) δ = 8.42 (d, J = 6.0 Hz, 1H), 7.88 (t, J = 8.8 Hz, 1H), 7.65 (d, J = 2.0 Hz, 1H), 7.46 (dd, J = 1.6, 12.0 Hz, 1H), 7.19 (br d, J = 7.6 Hz, 1H), 6.97 (dd, J = 2.4, 5.6 Hz, 1H), 4.81 (t, J = 5.2 Hz, 1H), 4.39-4.30 (m, 2H), 4.28- 4.17 (m, 2H), 3.76-3.66 (m, 2H), 2.81 (t, J = 6.4 Hz, 2H), 2.35 (s, 3H). |
| | 7-(4-(3-fluoro-4- | | |
| | (trifluoromethyl)phenoxy)pyridin- | | |
| | 2-yl)-3-(2-hydroxyethyl)-1- | | |
| | methyl-6,7-dihydroimidazo[1,5- | | |
| | α]pyrazin-8(5H)-one | | |
| 289 | 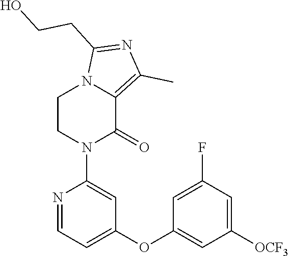 [SMILES: Cc1nc(CCO)n2c1C(=O)N(c1cc(Oc3cc(F)cc(OC(F)(F)F)c3)ccn1)CC2] | 467.2 | ¹H NMR (400 MHz, DMSO-d₆) δ = 8.39 (d, J = 5.6 Hz, 1H), 7.61 (d, J = 2.0 Hz, 1H), 7.33 (br d, J = 9.2 Hz, 1H), 7.30-7.22 (m, 1H), 7.17 (s, 1H), 6.90 (dd, J = 2.0, 5.6 Hz, 1H), 4.82 (t, J = 5.6 Hz, 1H), 4.40-4.28 (m, 2H), 4.28-4.17 (m, 2H), 3.70 (q, J = 6.4 Hz, 2H), 2.80 (t, J = 6.4 Hz, 2H), 2.35 (s, 3H). |
| | 7-(4-(3-fluoro-5- | | |
| | (trifluoromethoxy)phenoxy)pyridin- | | |
| | 2-yl)-3-(2-hydroxyethyl)-1- | | |
| | methyl-6,7-dihydroimidazo[1,5- | | |
| | α]pyrazin-8(5H)-one | | |
| 290 | 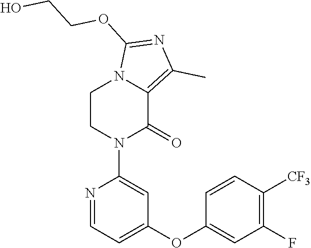 [SMILES: Cc1nc(OCCO)n2c1C(=O)N(c1cc(Oc3ccc(C(F)(F)F)c(F)c3)ccn1)CC2] | 467.2 | ¹H NMR (500 MHz, DMSO-d₆) δ = 8.41 (d, J = 5.5 Hz, 1H), 7.88 (t, J = 8.5 Hz, 1H), 7.62 (d, J = 2.5 Hz, 1H), 7.48-7.41 (m, 1H), 7.18 (br d, J = 8.0 Hz, 1H), 6.96 (dd, J = 2.0, 5.5 Hz, 1H), 4.94 (t, J = 5.5 Hz, 1H), 4.40- 4.30 (m, 4H), 4.00 (t, J = 5.5 Hz, 2H), 3.71 (q, J = 5.5 Hz, 2H), 2.33 (s, 3H). |
| | 7-(4-(3-fluoro-4- | | |
| | (trifluoromethyl)phenoxy)pyridin- | | |
| | 2-yl)-3-(2-hydroxyethoxy)-1- | | |
| | methyl-6,7-dihydroimidazo[1,5- | | |
| | α]pyrazin-8(5H)-one | | |
| 291 | 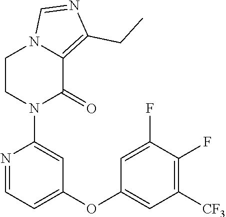 [SMILES: CCc1ncn2c1C(=O)N(c1cc(Oc3cc(F)c(F)c(C(F)(F)F)c3)ccn1)CC2] | 439.1 | ¹H NMR (400 MHz, DMSO-d₆) δ = 8.37 (d, J = 5.6 Hz, 1H), 7.93- 7.86 (m, 1H), 7.79 (s, 1H), 7.60- 7.52 (m, 2H), 6.89 (dd, J = 2.4, 5.6 Hz, 1H), 4.37- 4.28 (m, 4H), 2.81 (q, J = 7.6 Hz, 2H), 1.15 (t, J = 7.6 Hz, 3H). |
| | 7-(4-(3,4-difluoro-5- | | |
| | (trifluoromethyl)phenoxy)pyridin- | | |
| | 2-yl)-1-ethyl-6,7- | | |
| | dihydroimidazo[1,5-α]pyrazin- | | |
| | 8(5H)-one | | |
| 294 | 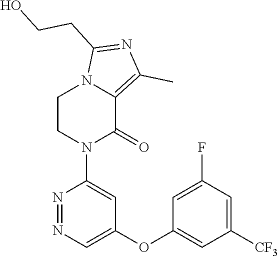 [SMILES: Cc1nc(CCO)n2c1C(=O)N(c1cc(Oc3cc(F)cc(C(F)(F)F)c3)cnn1)CC2] | 452.1 | ¹H NMR (400 MHz, DMSO-d₆) δ = 9.05 (d, J = 2.4 Hz, 1H), 7.74-7.60 (m, 4H), 4.83 (t, J = 5.2 Hz, 1H), 4.50-4.41 (m, 2H), 4.35-4.18 (m, 2H), 3.70 (q, J = 6.4 Hz, 2H), 2.81 (t, J = 6.4 Hz, 2H), 2.34 (s, 3H). |
| | 7-(5-(3-fluoro-5- | | |
| | (trifluoromethyl)phenoxy)pyridazin- | | |
| | 3-yl)-3-(2-hydroxyethyl)-1- | | |
| | methyl-6,7-dihydroimidazo[1,5- | | |
| | α]pyrazin-8(5H)-one | | |
| 295 | 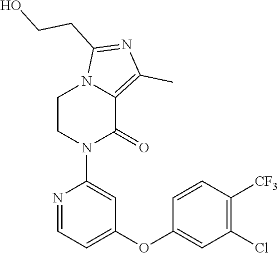 [SMILES: Cc1nc(CCO)n2c1C(=O)N(c1cc(Oc3ccc(C(F)(F)F)c(Cl)c3)ccn1)CC2] | 467.1 | ¹H NMR (400 MHz, DMSO-d₆) δ = 8.42 (d, J = 5.6 Hz, 1H), 7.95 (d, J = 8.8 Hz, 1H), 7.63 (dd, J = 2.4, 9.2 Hz, 2H), 7.32 (dd, J = 2.0, 8.8 Hz, 1H), 6.97 (dd, J = 2.4, 5.6 Hz, 1H), 4.81 (t, J = 5.2 Hz, 1H), 4.38-4.32 (m, 2H), 4.25-4.20 (m, 2H), 3.74-3.67 (m, 2H), 2.80 (t, J = 6.8 Hz, 2H), 2.35 (s, 3H). |
| | 7-(4-(3-chloro-4- | | |
| | (trifluoromethyl)phenoxy)pyridin- | | |
| | 2-yl)-3-(2-hydroxyethyl)-1- | | |
| | methyl-6,7-dihydroimidazo[1,5- | | |
| | α]pyrazin-8(5H)-one | | |
| 296 | 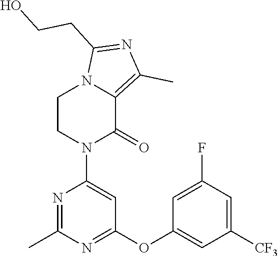 [SMILES: Cc1nc(Oc2cc(F)cc(C(F)(F)F)c2)cc(N2CCn3c(CCO)nc(C)c3C2=O)n1] | 466.1 | ¹H NMR (400 MHz, DMSO-d₆) δ = 7.68-7.57 (m, 3H), 7.55 (s, 1H), 4.84 (t, J = 5.6 Hz, 1H), 4.46 (dd, J = 4.4, 6.8 Hz, 2H), 4.27-4.17 (m, 2H), 3.78-3.66 (m, 2H), 2.81 (t, J = 6.8 Hz, 2H), 2.44 (s, 3H), 2.39 (s, 3H). |
| | 7-(6-(3-fluoro-5- | | |
| | (trifluoromethyl)phenoxy)-2- | | |
| | methylpyrimidin-4-yl)-3-(2- | | |
| | hydroxyethyl)-1-methyl-6,7- | | |
| | dihydroimidazo[1,5-α]pyrazin- | | |
| | 8(5H)-one | | |
| 297 | 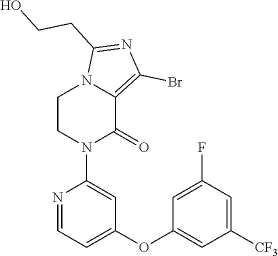 [SMILES: O=C1c2c(Br)nc(CCO)n2CCN1c1cc(Oc2cc(F)cc(C(F)(F)F)c2)ccn1] | 515.0 | ¹H NMR (400 MHz, DMSO-d₆) δ = 8.41 (d, J = 5.6 Hz, 1H), 7.65 (br d, J = 8.0 Hz, 1H), 7.60 (d, J = 2.4 Hz, 1H), 7.55 (br d, J = 9.6 Hz, 1H), 7.49 (s, 1H), 6.98-6.87 (m, 1H), 4.86 (t, J = 5.2 Hz, 1H), 4.42-4.35 (m, 2H), 4.34-4.27 (m, 2H), 3.70 (q, J = 6.0 Hz, 2H), 2.85 (t, J = 6.4 Hz, 2H). |
| | 1-bromo-7-(4-(3-fluoro-5- | | |
| | (trifluoromethyl)phenoxy)pyridin- | | |
| | 2-yl)-3-(2-hydroxyethyl)-6,7- | | |
| | dihydroimidazo[1,5-α]pyrazin- | | |
| | 8(5H)-one | | |
| 298 | 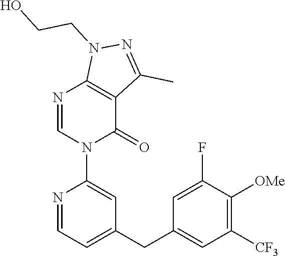 [SMILES: COc1c(F)cc(Cc2ccnc(-n3cnc4c(c(C)nn4CCO)c3=O)c2)cc1C(F)(F)F] | 478.1 | ¹H NMR (400 MHz, DMSO-d₆) δ = 8.54 (d, J = 5.2 Hz, 1H), 8.46 (s, 1H), 7.71 (s, 1H), 7.64 (dd, J = 1.6, 12.8 Hz, 1H), 7.52 (s, 1H), 7.49 (dd, J = 1.2, 5.2 Hz, 1H), 4.89 (t, J = 5.6 Hz, 1H), 4.28 (t, J = 5.6 Hz, 2H), 4.13 (s, 2H), 3.91 (d, J = 1.6 Hz, 3H), 3.79 (q, J = 5.6 Hz, 2H), 2.46 (s, 3H). |
| | 5-(4-(3-fluoro-4-methoxy-5- | | |
| | (trifluoromethyl)benzyl)pyridin- | | |
| | 2-yl)-1-(2-hydroxyethyl)-3- | | |
| | methyl-1,5-dihydro-4H- | | |
| | pyrazolo[3,4-d]pyrimidin-4-one | | |
| 299 | 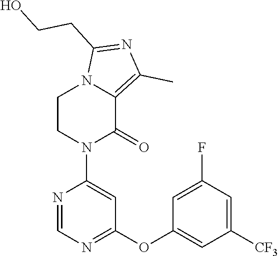 [SMILES: Cc1nc(CCO)n2c1C(=O)N(c1cc(Oc3cc(F)cc(C(F)(F)F)c3)ncn1)CC2] | 452.1 | ¹H NMR (400 MHz, DMSO-d₆) δ = 8.68 (s, 1H), 7.80 (s, 1H), 7.69-7.62 (m, 2H), 7.59 (s, 1H), 4.83 (t, J = 5.2 Hz, 1H), 4.51- 4.42 (m, 2H), 4.31-4.19 (m, 2H), 3.71 (q, J = 6.4 Hz, 2H), 2.82 (t, J = 6.4 Hz, 2H), 2.40 (s, 3H). |
| | 7-(6-(3-fluoro-5- | | |
| | (trifluoromethyl)phenoxy)pyrimidin- | | |
| | 4-yl)-3-(2-hydroxyethyl)-1- | | |
| | methyl-6,7-dihydroimidazo[1,5- | | |
| | α]pyrazin-8(5H)-one | | |
| 300 | 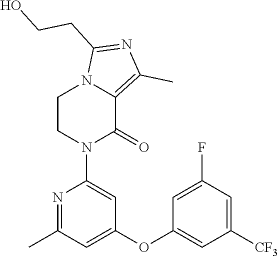 [SMILES: Cc1cc(Oc2cc(F)cc(C(F)(F)F)c2)cc(N2CCn3c(CCO)nc(C)c3C2=O)n1] | 465.1 | ¹H NMR (500 MHz, DMSO-d₆) δ = 7.62 (br d, J = 8.5 Hz, 1H), 7.50 (td, J = 2.5, 9.5 Hz, 1H), 7.47-7.43 (m, 2H), 6.78 (d, J = 2.0 Hz, 1H), 4.80 (t, J = 5.5 Hz, 1H), 4.38-4.32 (m, 2H), 4.24- 4.18 (m, 2H), 3.75-3.65 (m, 2H), 2.80 (t, J = 7 Hz, 2H), 2.43 (s, 3H), 2.34 (s, 3H). |
| | 7-(4-(3-fluoro-5- | | |
| | (trifluoromethyl)phenoxy)-6- | | |
| | methylpyridin-2-yl)-3-(2- | | |
| | hydroxyethyl)-1-methyl-6,7- | | |
| | dihydroimidazo[1,5-α]pyrazin- | | |
| | 8(5H)-one | | |
| 301 | 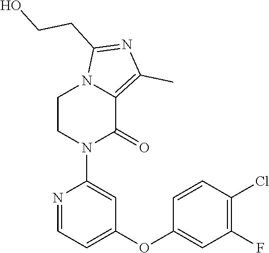 [SMILES: Cc1nc(CCO)n2c1C(=O)N(c1cc(Oc3ccc(Cl)c(F)c3)ccn1)CC2] | 417.1 | ¹H NMR (400 MHz, DMSO-d₆) δ = 8.35 (d, J = 5.6 Hz, 1H), 7.70 (t, J = 8.8 Hz, 1H), 7.54 (d, J = 2.0 Hz, 1H), 7.42 (dd, J = 2.4, 10.1 Hz, 1H), 7.10 (dd, J = 2.0, 8.8 Hz, 1H), 6.86 (dd, J = 2.0, 5.6 Hz, 1H), 4.83 (t, J = 5.2 Hz, 1H), 4.37-4.28 (m, 2H), 4.26-4.15 (m, 2H), 3.70 (q, J = 6.4 Hz, 2H), 2.80 (t, J = 6.4 Hz, 2H), 2.34 (s, 3H). |
| | 7-(4-(4-chloro-3- | | |
| | fluorophenoxy)pyridin-2-yl)-3- | | |
| | (2-hydroxyethyl)-1-methyl-6,7- | | |
| | dihydroimidazo[1,5-α]pyrazin- | | |
| | 8(5H)-one | | |
| 303 | 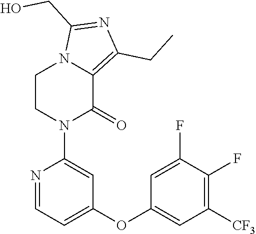 [SMILES: CCc1nc(CO)n2c1C(=O)N(c1cc(Oc3cc(F)c(F)c(C(F)(F)F)c3)ccn1)CC2] | 469.1 | ¹H NMR (400 MHz, DMSO-d₆) δ = 8.38 (d, J = 5.6 Hz, 1H), 7.95-7.88 (m, 1H), 7.60 (d, J = 2.4 Hz, 1H), 7.58-7.54 (m, 1H), 6.89 (dd, J = 2.0, 5.6 Hz, 1H), 5.48 (t, J = 5.6 Hz, 1H), 4.53 (d, J = 5.6 Hz, 2H), 4.36-4.28 (m, 4H), 2.80 (q, J = 7.6 Hz, 2H), 1.15 (t, J = 7.6 Hz, 3H). |
| | 7-(4-(3,4-difluoro-5- | | |
| | (trifluoromethyl)phenoxy)pyridin- | | |
| | 2-yl)-1-ethyl-3- | | |
| | (hydroxymethyl)-6,7- | | |
| | dihydroimidazo[1,5-α]pyrazin- | | |
| | 8(5H)-one | | |
| 304 | 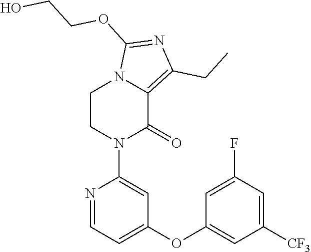 [SMILES: CCc1nc(OCCO)n2c1C(=O)N(c1cc(Oc3cc(F)cc(C(F)(F)F)c3)ccn1)CC2] | 481.1 | ¹H NMR (400 MHz, DMSO-d₆) δ = 8.38 (d, J = 5.6 Hz, 1H), 7.66-7.61 (m, 1H), 7.58 (d, J = 2.0 Hz, 1H), 7.53 (td, J = 2.4, 10.0 Hz, 1H), 7.49-7.45 (m, 1H), 6.90 (dd, J = 2.0, 5.6 Hz, 1H), 5.19-4.69 (m, 1H), 4.40- 4.28 (m, 4H), 4.06-3.96 (m, 2H), 3.71 (t, J = 4.8 Hz, 2H), 2.76 (q, J = 7.6 Hz, 2H), 1.12 (t, J = 7.6 Hz, 3H). |
| | 1-ethyl-7-(4-(3-fluoro-5- | | |
| | (trifluoromethyl)phenoxy)pyridin- | | |
| | 2-yl)-3-(2-hydroxyethoxy)- | | |
| | 6,7-dihydroimidazo[1,5- | | |
| | α]pyrazin-8(5H)-one | | |
| 305 | 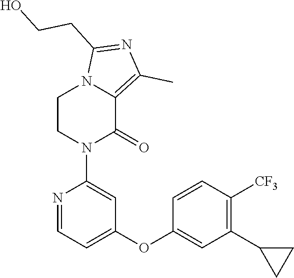 [SMILES: Cc1nc(CCO)n2c1C(=O)N(c1cc(Oc3ccc(C(F)(F)F)c(C4CC4)c3)ccn1)CC2] | 473.3 | ¹H NMR (400 MHz, DMSO-d₆) δ = 8.37 (d, J = 5.6 Hz, 1H), 7.76 (d, J = 8.8 Hz, 1H), 7.51 (d, J = 2.4 Hz, 1H), 7.13-7.08 (m, 1H), 6.97-6.92 (m, 1H), 6.90-6.84 (m, 1H), 4.81 (t, J = 5.2 Hz, 1H), 4.35-4.29 (m, 2H), 4.25-4.18 (m, 2H), 3.76-3.64 (m, 2H), 2.80 (t, J = 6.4 Hz, 2H), 2.35 (s, 3H), 2.19-2.07 (m, 1H), 1.08-1.00 (m, 2H), 0.93- 0.80 (m, 2H). |
| | 7-(4-(3-cyclopropyl-4- | | |
| | (trifluoromethyl)phenoxy)pyridin- | | |
| | 2-yl)-3-(2-hydroxyethyl)-1- | | |
| | methyl-6,7-dihydroimidazo[1,5- | | |
| | α]pyrazin-8(5H)-one | | |
| 306 | 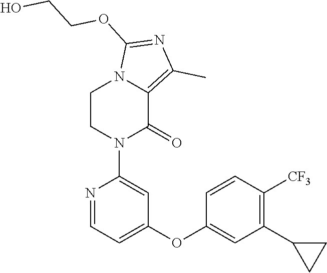 [SMILES: Cc1nc(OCCO)n2c1C(=O)N(c1cc(Oc3ccc(C(F)(F)F)c(C4CC4)c3)ccn1)CC2] | 489.3 | ¹H NMR (400 MHz, DMSO-d₆) δ = 8.35 (d, J = 5.6 Hz, 1H), 7.76 (d, J = 8.8 Hz, 1H), 7.48 (d, J = 2.0 Hz, 1H), 7.13-7.06 (m, 1H), 6.94 (d, J = 1.2 Hz, 1H), 6.88-6.83 (m, 1H), 4.94 (t, J = 5.6 Hz, 1H), 4.38-4.24 (m, 4H), 3.99 (t, J = 5.2 Hz, 2H), 3.70 (q, J = 5.2 Hz, 2H), 2.33 (s, 3H), 2.18-2.10 (m, 1H), 1.07- 1.01 (m, 2H), 0.91-0.85 (m, 2H). |
| | 7-(4-(3-cyclopropyl-4- | | |
| | (trifluoromethyl)phenoxy)pyridin- | | |
| | 2-yl)-3-(2-hydroxyethoxy)-1- | | |
| | methyl-6,7-dihydroimidazo[1,5- | | |
| | α]pyrazin-8(5H)-one | | |
| 307 | 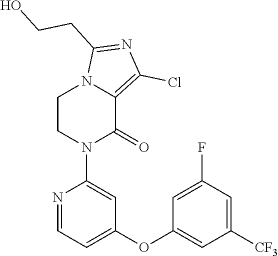 [SMILES: O=C1c2c(Cl)nc(CCO)n2CCN1c1cc(Oc2cc(F)cc(C(F)(F)F)c2)ccn1] | 471.0 | ¹H NMR (400 MHz, DMSO-d₆) δ = 8.41 (d, J = 5.6 Hz, 1H), 7.75-7.39 (m, 4H), 6.94 (dd, J = 2.4, 5.6 Hz, 1H), 4.86 (t, J = 5.2 Hz, 1H), 4.47-4.36 (m, 2H), 4.34-4.20 (m, 2H), 3.70 (q, J = 6.0 Hz, 2H), 2.84 (t, J = 6.4 Hz, 2H). |
| | 1-chloro-7-(4-(3-fluoro-5- | | |
| | (trifluoromethyl)phenoxy)pyridin- | | |
| | 2-yl)-3-(2-hydroxyethyl)-6,7- | | |
| | dihydroimidazo[1,5-α]pyrazin- | | |
| | 8(5H)-one | | |
| 308 | 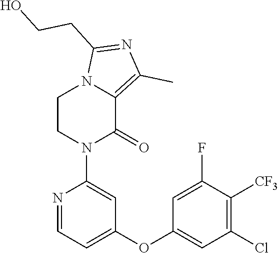 [SMILES: Cc1nc(CCO)n2c1C(=O)N(c1cc(Oc3cc(F)c(C(F)(F)F)c(Cl)c3)ccn1)CC2] | 485.0 | ¹H NMR (400 MHz, DMSO-d₆) δ = 8.44 (d, J = 5.6 Hz, 1H), 7.70 (d, J = 2.0 Hz, 1H), 7.49- 7.33 (m, 2H), 7.03 (dd, J = 2.4, 5.6 Hz, 1H), 4.81 (t, J = 5.6 Hz, 1H), 4.39-4.31 (m, 2H), 4.29- 4.18 (m, 2H), 3.71 (q, J = 6.4 Hz, 2H), 2.81 (t, J = 6.4 Hz, 2H), 2.36 (s, 3H). |
| | 7-(4-(3-chloro-5-fluoro-4- | | |
| | (trifluoromethyl)phenoxy)pyridin- | | |
| | 2-yl)-3-(2-hydroxyethyl)-1- | | |
| | methyl-6,7-dihydroimidazo[1,5- | | |
| | α]pyrazin-8(5H)-one | | |
| 309 | 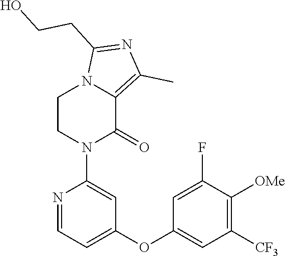 [SMILES: COc1c(F)cc(Oc2ccnc(N3CCn4c(CCO)nc(C)c4C3=O)c2)cc1C(F)(F)F] | 479.2 | ¹H NMR (400 MHz, DMSO-d₆) δ = 8.33 (d, J = 5.2 Hz, 1H), 7.81 (s, 1H), 7.60 (dd, J = 1.6, 12.8 Hz, 1H), 7.49 (s, 1H), 7.11 (dd, J = 1.2, 5.2 Hz, 1H), 4.82 (t, J = 5.2 Hz, 1H), 4.31-4.17 (m, 4H), 4.05 (s, 2H), 3.91 (d, J = 1.6 Hz, 3H), 3.73-3.68 (m, 2H), 2.80 (t, J = 6.4 Hz, 2H), 2.38 (s, 3H). |
| | 7-(4-(3-fluoro-4-methoxy-5- | | |
| | (trifluoromethyl)benzyl)pyridin- | | |
| | 2-yl)-3-(2-hydroxyethyl)-1- | | |
| | methyl-6,7-dihydroimidazo[1,5- | | |
| | α]pyrazin-8(5H)-one | | |
| 310 | 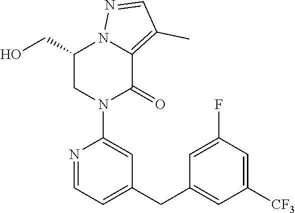 [SMILES: Cc1cnn2c1C(=O)N(c1cc(Cc3cc(F)cc(C(F)(F)F)c3)ccn1)C[C@@H]2CO] | 435.1 | ¹H NMR (400 MHz, DMSO-d₆) δ = 8.39 (d, J = 5.2 Hz, 1H), 7.88 (s, 1H), 7.60 (s, 1H), 7.58- 7.51 (m, 2H), 7.50 (s, 1H), 7.17 (d, J = 5.2 Hz, 1H), 5.19 (t, J = 6 Hz, 1H), 4.58-4.41 (m, 3H), 4.15 (s, 2H), 3.91-3.60 (m, 2H), 2.27 (s, 3H). |
| | (R)-5-(4-(3-fluoro-5- | | |
| | (trifluoromethyl)benzyl)pyridin- | | |
| | 2-yl)-7-(hydroxymethyl)-3- | | |
| | methyl-6,7-dihydropyrazolo[1,5- | | |
| | α]pyrazin-4(5H)-one | | |
| 311 | 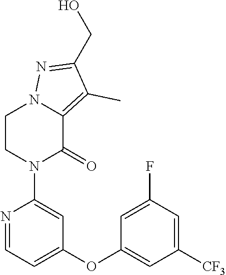 [SMILES: Cc1c(CO)nn2c1C(=O)N(c1cc(Oc3cc(F)cc(C(F)(F)F)c3)ccn1)CC2] | 437.1 | ¹H NMR (400 MHz, DMSO-d6) δ = 8.43 (d, J = 5.6 Hz, 1H), 7.66 (d, J = 8.4 Hz, 1H), 7.62 (d, J = 2.4 Hz, 1H), 7.59-7.53 (m, 1H), 7.50 (s, 1H), 6.99-6.92 (m, 1H), 5.01 (t, J = 5.6 Hz, 1H), 4.46-4.41 (m, 4H), 4.40- 4.37 (m, 2H), 2.26 (s, 3H). |
| | 5-(4-(3-fluoro-5- | | |
| | (trifluoromethyl)phenoxy)pyridin- | | |
| | 2-yl)-2-(hydroxymethyl)-3- | | |
| | methyl-6,7-dihydropyrazolo[1,5- | | |
| | α]pyrazin-4(5H)-one | | |
| 312 | 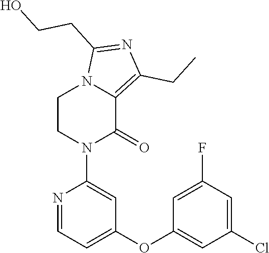 [SMILES: CCc1nc(CCO)n2c1C(=O)N(c1cc(Oc3cc(F)cc(Cl)c3)ccn1)CC2] | 431.1 | ¹H NMR (400 MHz, DMSO-d₆) δ = 8.38 (d, J = 5.6 Hz, 1H), 7.58 (d, J = 2.4 Hz, 1H), 7.41- 7.33 (m, 1H), 7.24-7.15 (m, 2H), 6.91-6.86 (m, 1H), 4.88- 4.78 (m, 1H), 4.36-4.29 (m, 2H), 4.25-4.19 (m, 2H), 3.73- 3.67 (m, 2H), 2.84-2.76 (m, 4H), 1.16-1.11 (m, 3H). |
| | 7-(4-(3-chloro-5- | | |
| | fluorophenoxy)pyridin-2-yl)-1- | | |
| | ethyl-3-(2-hydroxyethyl)-6,7- | | |
| | dihydroimidazo[1,5-α]pyrazin- | | |
| | 8(5H)-one | | |
| 314 | 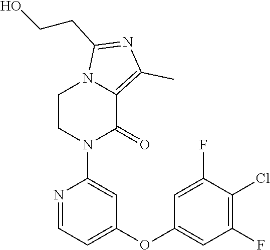 [SMILES: Cc1nc(CCO)n2c1C(=O)N(c1cc(Oc3cc(F)c(Cl)c(F)c3)ccn1)CC2] | 435.1 | ¹H NMR (400 MHz, DMSO- d₆) δ = 8.38 (d, J = 5.6 Hz, 1H), 7.60 (d, J = 2.0 Hz, 1H), 7.35 (d, J = 8.0 Hz, 2H), 6.92 (dd, J = 2.4, 6.0 Hz, 1H), 4.81 (t, J = 5.6 Hz, 1H), 4.36-4.31 (m, 2H), 4.26-4.19 (m, 2H), 3.70 (q, J = 6.4 Hz, 2H), 2.80 (t, J = 6.4 Hz, 2H), 2.35 (s, 3H). |
| | 7-(4-(4-chloro-3,5- | | |
| | difluorophenoxy)pyridin-2-yl)-3- | | |
| | (2-hydroxyethyl)-1-methyl-6,7- | | |
| | dihydroimidazo[1,5-α]pyrazin- | | |
| | 8(5H)-one | | |
| 315 | 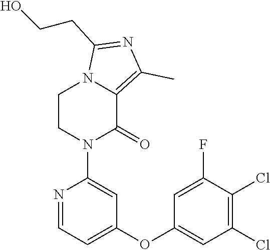 [SMILES: Cc1nc(CCO)n2c1C(=O)N(c1cc(Oc3cc(F)c(Cl)c(Cl)c3)ccn1)CC2] | 450.9 | ¹H NMR (400 MHz, DMSO-d₆) δ = 8.38 (d, J = 5.6 Hz, 1H), 7.60 (d, J = 2.4 Hz, 1H), 7.51- 7.46 (m, 2H), 6.92 (dd, J = 2.4, 5.6 Hz, 1H), 4.81 (s, 1H), 4.36- 4.30 (m, 2H), 4.26-4.18 (m, 2H), 3.70 (s, 2H), 2.80 (t, J = 6.8 Hz, 2H), 2.35 (s, 3H). |
| | 7-(4-(3,4-dichloro-5- | | |
| | fluorophenoxy)pyridin-2-yl)-3- | | |
| | (2-hydroxyethyl)-1-methyl-6,7- | | |
| | dihydroimidazo[1,5-α]pyrazin- | | |
| | 8(5H)-one | | |
| 317 | 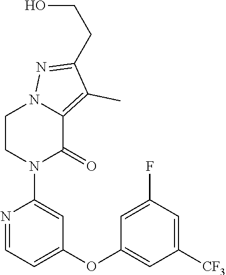 [SMILES: Cc1c(CCO)nn2c1C(=O)N(c1cc(Oc3cc(F)cc(C(F)(F)F)c3)ccn1)CC2] | 451.2 | ¹H NMR (400 MHz, DMSO-d₆) δ = 8.42 (d, J = 5.6 Hz, 1H), 7.66 (d, J = 8.4 Hz, 1H), 7.60 (d, J = 2.4 Hz, 1H), 7.59-7.54 (m, 1H), 7.50 (s, 1H), 6.95 (dd, J = 2.4, 5.6 Hz, 1H), 4.66 (t, J = 5.6 Hz, 1H), 4.47-4.40 (m, 2H), 4.39-4.33 (m, 2H), 3.63- 3.56 (m, 2H), 2.70 (t, J = 7.2 Hz, 2H), 2.20 (s, 3H). |
| | 5-(4-(3-fluoro-5- | | |
| | (trifluoromethyl)phenoxy)pyridin- | | |
| | 2-yl)-2-(2-hydroxyethyl)-3- | | |
| | methyl-6,7-dihydropyrazolo[1,5- | | |
| | α]pyrazin-4(5H)-one | | |
| 318 | 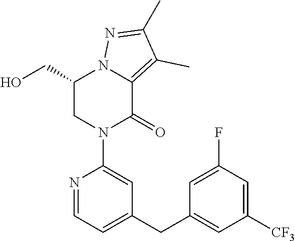 [SMILES: Cc1nn2c(c1C)C(=O)N(c1cc(Cc3cc(F)cc(C(F)(F)F)c3)ccn1)C[C@@H]2CO] | 449.2 | ¹H NMR (400 MHz, DMSO-d₆) δ = 8.38 (d, J = 5.2 Hz, 1H), 7.85 (s, 1H), 7.60 (s, 1H), 7.55- 7.51 (m, 2H), 7.16-7.14 (m, 1H), 5.18-5.16 (m, 1H), 4.45- 4.38 (m, 3H), 4.15 (s, 2H), 3.78-3.66 (m, 2H), 2.19 (s, 3H), 2.15 (s, 3H). |
| | (R)-5-(4-(3-fluoro-5- | | |
| | (trifluoromethyl)benzyl)pyridin- | | |
| | 2-yl)-7-(hydroxymethyl)-2,3- | | |
| | dimethyl-6,7- | | |
| | dihydropyrazolo[1,5-α]pyrazin- | | |
| | 4(5H)-one | | |
| 319 | 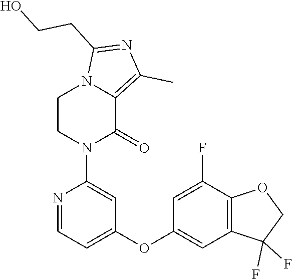 [SMILES: Cc1nc(CCO)n2c1C(=O)N(c1cc(Oc3cc(F)c4c(c3)C(F)(F)CO4)ccn1)CC2] | 461.1 | ¹H NMR (400 MHz, DMSO-d₆) δ = 8.33 (d, J = 6.0 Hz, 1H), 7.58 (dd, J = 2.0, 11.2 Hz, 1H), 7.51 (d, J = 2.0 Hz, 1H), 7.49- 7.47 (m, 1H), 6.79 (dd, J = 2.0, 6.0 Hz, 1H), 5.00 (t, J = 16.4 Hz, 2H), 4.80 (t, J = 5.2 Hz, 1H), 4.37-4.27 (m, 2H), 4.27- 4.17 (m, 2H), 3.74-3.65 (m, 2H), 2.80 (t, J = 6.4 Hz, 2H), 2.35 (s, 3H). |
| | 3-(2-hydroxyethyl)-1-methyl-7- | | |
| | (4-((3,3,7-trifluoro-2,3- | | |
| | dihydrobenzofuran-5- | | |
| | yl)oxy)pyridin-2-yl)-6,7- | | |
| | dihydroimidazo[1,5-α]pyrazin- | | |
| | 8(5H)-one | | |
| 320 | 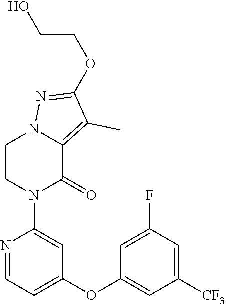 [SMILES: Cc1c(OCCO)nn2c1C(=O)N(c1cc(Oc3cc(F)cc(C(F)(F)F)c3)ccn1)CC2] | 467.2 | ¹H NMR (400 MHz, DMSO-d₆) δ = 8.43 (d, J = 6.0 Hz, 1H), 7.65 (d, J = 8.4 Hz, 1H), 7.61 (d, J = 2.4 Hz, 1H), 7.58-7.52 (m, 1H), 7.49 (s, 1H), 6.96 (dd, J = 2.0, 5.6 Hz, 1H), 4.84 (t, J = 5.2 Hz, 1H), 4.45-4.39 (m, 2H), 4.28-4.23 (m, 2H), 4.16 (t, J = 5.0 Hz, 2H), 3.74-3.65 (m, 2H), 2.09 (s, 3H). |
| | 5-(4-(3-fluoro-5- | | |
| | (trifluoromethyl)phenoxy)pyridin- | | |
| | 2-yl)-2-(2-hydroxyethoxy)-3- | | |
| | methyl-6,7-dihydropyrazolo[1,5- | | |
| | α]pyrazin-4(5H)-one | | |
| 321 | 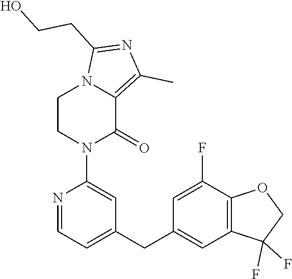 [SMILES: Cc1nc(CCO)n2c1C(=O)N(c1cc(Cc3cc(F)c4c(c3)C(F)(F)CO4)ccn1)CC2] | 459.3 | ¹H NMR (400 MHz, DMSO-d₆) δ = 8.33 (d, J = 5.2 Hz, 1H), 7.80 (s, 1H), 7.48 (d, J = 12.0 Hz, 1H), 7.44 (s, 1H), 7.10 (dd, J = 1.6, 5.2 Hz, 1H), 4.92 (t, J = 16.4 Hz, 2H), 4.82 (t, J = 5.2 Hz, 1H), 4.31-4.16 (m, 4H), 4.02 (s, 2H), 3.75-3.65 (m, 2H), 2.80 (t, J = 6.4 Hz, 2H), 2.37 (s, 3H). |
| | 3-(2-hydroxyethyl)-1-methyl-7- | | |
| | (4-((3,3,7-trifluoro-2,3- | | |
| | dihydrobenzofuran-5- | | |
| | yl)methyl)pyridin-2-yl)-6,7- | | |
| | dihydroimidazo[1,5-α]pyrazin- | | |
| | 8(5H)-one | | |
| 323 | 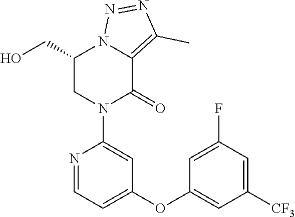 [SMILES: Cc1nnn2c1C(=O)N(c1cc(Oc3cc(F)cc(C(F)(F)F)c3)ccn1)C[C@@H]2CO] | 438.3 | ¹H NMR (400 MHz, DMSO-d₆) δ = 8.44 (d, J = 5.6 Hz, 1H), 7.67-7.65 (m, 1H), 7.62 (d, J = 2.4 Hz, 1H), 7.58-7.55 (m, 1H), 7.52 (s, 1H), 6.99-6.97 (m, 1H), 5.33-5.30 (m, 1H), 4.95-4.92 (m, 1H), 4.62-4.52 (m, 2H), 3.92-3.88 (m, 2H), 2.46 (s, 3H). |
| | (R)-5-(4-(3-fluoro-5- | | |
| | (trifluoromethyl)phenoxy)pyridin- | | |
| | 2-yl)-7-(hydroxymethyl)-3- | | |
| | methyl-6,7-dihydro- | | |
| | [1,2,3]triazolo[1,5-α]pyrazin- | | |
| | 4(5H)-one | | |
| 324 | 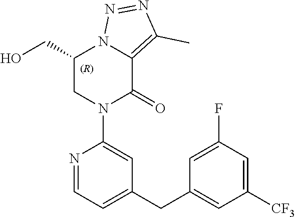 | 436.1 | ¹H NMR (400 MHz, DMSO-d₆) δ = 8.40 (d, J = 4.4 Hz, 1H), 7.88 (s, 1H), 7.60 (s, 1H), 7.56- 7.52 (m, 2H), 7.20-7.19 (m, 1H), 5.30 (s, 1H), 4.93-4.91 (m, 1H), 4.52 (d, J = 4.0 Hz, 2H), 4.16 (s, 2H), 3.89 (s, 2H), 2.48 (s, 3H). |
| | (R)-5-(4-(3-fluoro-5- | | |
| | (trifluoromethyl)benzyl)pyridin- | | |
| | 2-yl)-7-(hydroxymethyl)-3- | | |
| | methyl-6,7-dihydro- | | |
| | [1,2,3]triazolo[1,5-α]pyrazin- | | |
| | 4(5H)-one | | |
| 326 | 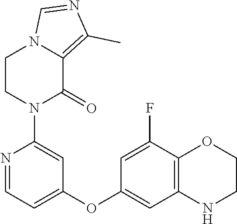 [SMILES: Cc1ncn2c1C(=O)N(c1cc(Oc3cc(F)c4c(c3)NCCO4)ccn1)CC2] | 396.1 | ¹H NMR (400 MHz, DMSO-d₆) δ = 8.29 (d, J = 5.6 Hz, 1H), 7.78 (s, 1H), 7.43 (d, J = 2.4 Hz, 1H), 6.77 (dd, J = 2.0, 5.6 Hz, 1H), 6.38 (br s, 1H), 6.28 (dd, J = 2.8, 11.6 Hz, 1H), 6.19 (dd, J = 1.6, 2.4 Hz, 1H), 4.31 (s, 4H), 4.16 (t, J = 4.0 Hz, 2H), 3.38- 3.33 (m, 2H), 2.38 (s, 3H). |
| | 7-(4-((8-fluoro-3,4-dihydro-2H- | | |
| | benzo[b][1,4]oxazin-6- | | |
| | yl)oxy)pyridin-2-yl)-1-methyl- | | |
| | 6,7-dihydroimidazo[1,5- | | |
| | α]pyrazin-8(5H)-one | | |
| 327 | 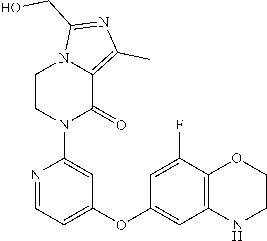 [SMILES: Cc1nc(CO)n2c1C(=O)N(c1cc(Oc3cc(F)c4c(c3)NCCO4)ccn1)CC2] | 426.1 | ¹H NMR (500 MHz, DMSO-d₆) δ = 8.30 (d, J = 5.5 Hz, 1H), 7.46 (d, J = 2.5 Hz, 1H), 6.77 (dd, J = 2.0, 5.5 Hz, 1H), 6.38 (br s, 1H), 6.28 (dd, J = 2.5, 11.0 Hz, 1H), 6.22-6.15 (m, 1H), 5.46 (t, J = 5.5 Hz, 1H), 4.51 (d, J = 6.0 Hz, 2H), 4.35- 4.25 (m, 4H), 4.16 (t, J = 4.5 Hz, 2H), 3.34 (br s, 2H), 2.36 (s, 3H). |
| | 7-(4-((8-fluoro-3,4-dihydro-2H- | | |
| | benzo[b][1,4]oxazin-6- | | |
| | yl)oxy)pyridin-2-yl)-3- | | |
| | (hydroxymethyl)-1-methyl-6,7- | | |
| | dihydroimidazo[1,5-α]pyrazin- | | |
| | 8(5H)-one | | |
| 331 | 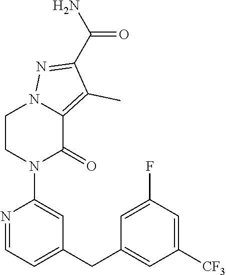 [SMILES: Cc1c(C(N)=O)nn2c1C(=O)N(c1cc(Cc3cc(F)cc(C(F)(F)F)c3)ccn1)CC2] | 448.1 | ¹H NMR (400 MHz, CDCl₃) δ = 8.37 (d, J = 4.8 Hz, 1H), 7.97 (s, 1H), 7.28 (br s, 1H), 7.22 (br d, J = 8.4 Hz, 1H), 7.10 (br d, J = 8.8 Hz, 1H), 6.91 (dd, J = 1.2, 5.2 Hz, 1H), 6.75 (br s, 1H), 5.49-5.30 (m, 1H), 4.61-4.55 (m, 2H), 4.51-4.46 (m, 2H), 4.08 (s, 2H), 2.70 (s, 3H). |
| | 5-(4-(3-fluoro-5- | | |
| | (trifluoromethyl)benzyl)pyridin- | | |
| | 2-yl)-3-methyl-4-oxo-4,5,6,7- | | |
| | tetrahydropyrazolo[1,5- | | |
| | α]pyrazine-2-carboxamide | | |
| 332 | 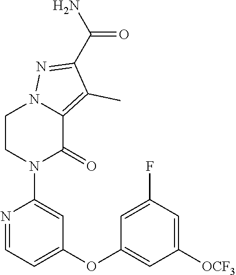 [SMILES: Cc1c(C(N)=O)nn2c1C(=O)N(c1cc(Oc3cc(F)cc(OC(F)(F)F)c3)ccn1)CC2] | 466.1 | ¹H NMR (400 MHz, DMSO-d₆) δ = 8.44 (d, J = 5.6 Hz, 1H), 7.62 (d, J = 2.4 Hz, 1H), 7.54 (s, 1H), 7.36 (d, J = 9.2 Hz, 1H), 7.32-7.26 (m, 2H), 7.20 (s, 1H), 6.97 (dd, J = 2.4, 5.6 Hz, 1H), 4.48 (s, 4H), 2.48 (s, 3H). |
| | 5-(4-(3-fluoro-5- | | |
| | (trifluoromethoxy)phenoxy)pyridin- | | |
| | 2-yl)-3-methyl-4-oxo-4,5,6,7- | | |
| | tetrahydropyrazolo[1,5- | | |
| | α]pyrazine-2-carboxamide | | |
| 333 | 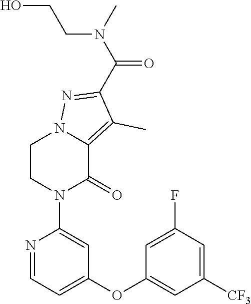 [SMILES: Cc1c(C(=O)N(C)CCO)nn2c1C(=O)N(c1cc(Oc3cc(F)cc(C(F)(F)F)c3)ccn1)CC2] | 508.2 | ¹H NMR (400 MHz, DMSO-d₆) δ = 8.44 (d, J = 5.6 Hz, 1H), 7.66 (br d, J = 8.0 Hz, 1H), 7.61 (s, 1H), 7.59-7.54 (m, 1H), 7.51 (s, 1H), 6.97 (br d, J = 5.6 Hz, 1H), 4.83-4.61 (m, 1H), 4.51- 4.42 (m, 4H), 3.60 (q, J = 6.0 Hz, 1H), 3.55-3.45 (m, 3H), 3.11-2.97 (m, 3H), 2.26 (d, J = 5.2 Hz, 3H). |
| | 5-(4-(3-fluoro-5- | | |
| | (trifluoromethyl)phenoxy)pyridin- | | |
| | 2-yl)-N-(2-hydroxyethyl)-N,3- | | |
| | dimethyl-4-oxo-4,5,6,7- | | |
| | tetrahydropyrazolo[1,5- | | |
| | α]pyrazine-2-carboxamide | | |
| 336 | 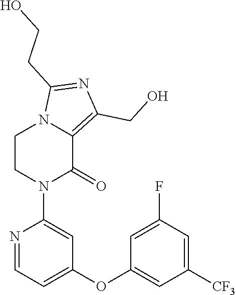 [SMILES: O=C1c2c(CO)nc(CCO)n2CCN1c1cc(Oc2cc(F)cc(C(F)(F)F)c2)ccn1] | 467.1 | ¹H NMR (400 MHz, DMSO-d₆) δ = 8.41 (d, J = 6.0 Hz, 1H), 7.64 (br d, J = 8.4 Hz, 1H), 7.60 (d, J = 2.4 Hz, 1H), 7.55 (br d, J = 9.6 Hz, 1H), 7.49 (s, 1H), 7.04-6.83 (m, 1H), 4.97-4.88 (m, 1H), 4.86-4.78 (m, 1H), 4.58 (d, J = 6.0 Hz, 2H), 4.42- 4.33 (m, 2H), 4.32-4.21 (m, 2H), 3.79-3.66 (m, 2H), 2.84 (t, J = 6.4 Hz, 2H). |
| | 7-(4-(3-fluoro-5- | | |
| | (trifluoromethyl)phenoxy)pyridin- | | |
| | 2-yl)-3-(2-hydroxyethyl)-1- | | |
| | (hydroxymethyl)-6,7- | | |
| | dihydroimidazo[1,5-α]pyrazin- | | |
| | 8(5H)-one | | |
| 337 | 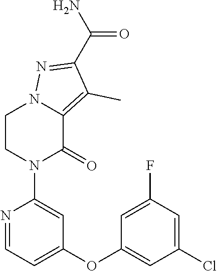 [SMILES: Cc1c(C(N)=O)nn2c1C(=O)N(c1cc(Oc3cc(F)cc(Cl)c3)ccn1)CC2] | 416.0 | ¹H NMR (400 MHz, DMSO-d₆) δ = 8.42 (d, J = 5.6 Hz, 1H), 7.59 (d, J = 2.0 Hz, 1H), 7.53 (s, 1H), 7.42-7.39 (m, 1H), 7.27- 7.20 (m, 3H), 6.95 (dd, J = 2.4, 5.6 Hz, 1H), 4.48 (s, 4H), 2.48 (s, 3H). |
| | 5-(4-(3-chloro-5- | | |
| | fluorophenoxy)pyridin-2-yl)-3- | | |
| | methyl-4-oxo-4,5,6,7- | | |
| | tetrahydropyrazolo[1,5- | | |
| | α]pyrazine-2-carboxamide | | |
| 338 | 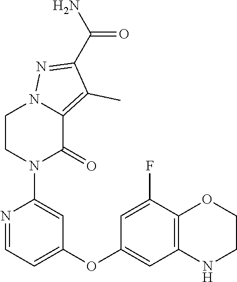 [SMILES: Cc1c(C(N)=O)nn2c1C(=O)N(c1cc(Oc3cc(F)c4c(c3)NCCO4)ccn1)CC2] | 439.1 | ¹H NMR (400 MHz, DMSO-d₆) δ = 8.33 (d, J = 6.0 Hz, 1H), 7.53 (s, 1H), 7.45 (d, J = 2.0 Hz, 1H), 7.27 (s, 1H), 6.82 (dd, J = 2.0, 5.6 Hz, 1H), 6.40 (br s, 1H), 6.29 (dd, J = 2.8, 11.2 Hz, 1H), 6.22-6.17 (m, 1H), 4.46 (br d, J = 4.0 Hz, 4H), 4.16 (t, J = 4.0 Hz, 2H), 3.34 (br d, J = 2.8 Hz, 2H), 2.48 (s, 3H). |
| | 5-(4-((8-fluoro-3,4-dihydro-2H- | | |
| | benzo[b][1,4]oxazin-6- | | |
| | yl)oxy)pyridin-2-yl)-3-methyl-4- | | |
| | oxo-4,5,6,7- | | |
| | tetrahydropyrazolo[1,5- | | |
| | α]pyrazine-2-carboxamide | | |
| 339 | 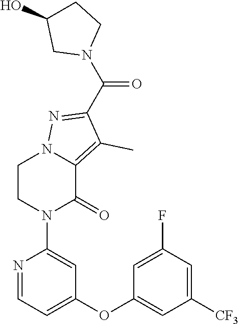 [SMILES: Cc1c(C(=O)N2CC[C@H](O)C2)nn2c1C(=O)N(c1cc(Oc3cc(F)cc(C(F)(F)F)c3)ccn1)CC2] | 520.1 | ¹H NMR (400 MHz, DMSO-d₆) δ = 8.44 (d, J = 5.6 Hz, 1H), 7.66 (d, J = 8.4 Hz, 1H), 7.62 (d, J = 2.0 Hz, 1H), 7.59-7.54 (m, 1H), 7.51 (s, 1H), 6.97 (dd, J = 2.4, 5.6 Hz, 1H), 4.96 (dd, J = 3.2, 7.6 Hz, 1H), 4.49 (s, 4H), 4.34-4.26 (m, 1H), 3.79-3.48 (m, 4H), 2.38 (d, J = 3.6 Hz, 3H), 1.97-1.87 (m, 1H), 1.86- 1.76 (m, 1H). |
| | (S)-5-(4-(3-fluoro-5- | | |
| | (trifluoromethyl)phenoxy)pyridin- | | |
| | 2-yl)-2-(3-hydroxypyrrolidine- | | |
| | 1-carbonyl)-3-methyl-6,7- | | |
| | dihydropyrazolo[1,5-α]pyrazin- | | |
| | 4(5H)-one | | |
| 340 | 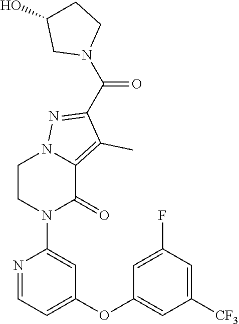 [SMILES: Cc1c(C(=O)N2CC[C@@H](O)C2)nn2c1C(=O)N(c1cc(Oc3cc(F)cc(C(F)(F)F)c3)ccn1)CC2] | 520.1 | ¹H NMR (400 MHz, DMSO-d₆) δ = 8.44 (d, J = 5.6 Hz, 1H), 7.67 (br d, J = 8.4 Hz, 1H), 7.62 (d, J = 2.0 Hz, 1H), 7.57 (br d, J = 9.6 Hz, 1H), 7.51 (s, 1H), 6.97 (dd, J = 2.4, 5.6 Hz, 1H), 4.96 (dd, J = 3.2, 8.0 Hz, 1H), 4.49 (s, 4H), 4.36-4.22 (m, 1H), 3.80-3.67 (m, 1.5H), 3.63-3.46 (m, 2H), 3.45-3.37 (m, 0.5H), 2.38 (d, J = 3.6 Hz, 3H), 2.01- 1.70 (m, 2H) |
| | (R)-5-(4-(3-fluoro-5- | | |
| | (trifluoromethyl)phenoxy)pyridin- | | |
| | 2-yl)-2-(3-hydroxypyrrolidine- | | |
| | 1-carbonyl)-3-methyl-6,7- | | |
| | dihydropyrazolo[1,5-α]pyrazin- | | |
| | 4(5H)-one | | |
| 342 | 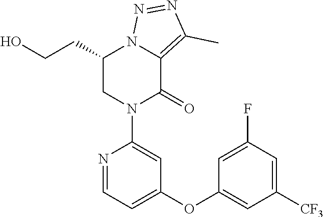 [SMILES: Cc1nnn2c1C(=O)N(c1cc(Oc3cc(F)cc(C(F)(F)F)c3)ccn1)C[C@@H]2CCO] | 452.1 | ¹H NMR (400 MHz, DMSO-d₆) δ = 8.44 (d, J = 5.6 Hz, 1H), 7.67 (br d, J = 8.4 Hz, 1H), 7.63 (d, J = 2.4 Hz, 1H), 7.58 (td, J = 2.0, 9.6 Hz, 1H), 7.52 (s, 1H), 6.99 (dd, J = 2.4, 5.6 Hz, 1H), 5.06-5.00 (m, 1H), 4.77 (t, J = 4.8 Hz, 1H), 4.62-4.48 (m, 2H), 3.58 (q, J = 5.6 Hz, 2H), 2.46 (s, 3H), 2.30-2.19 (m, 1H), 2.03- 1.93 (m, 1H). |
| | (S)-5-(4-(3-fluoro-5- | | |
| | (trifluoromethyl)phenoxy)pyridin- | | |
| | 2-yl)-7-(2-hydroxyethyl)-3- | | |
| | methyl-6,7-dihydro- | | |
| | [1,2,3]triazolo[1,5-α]pyrazin- | | |
| | 4(5H)-one | | |
| 343 | 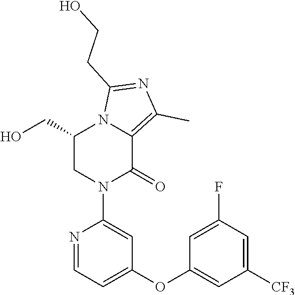 [SMILES: Cc1nc(CCO)n2c1C(=O)N(c1cc(Oc3cc(F)cc(C(F)(F)F)c3)ccn1)C[C@@H]2CO] | 481.2 | ¹H NMR (400 MHz, DMSO-d₆) δ = 8.38 (d, J = 5.6 Hz, 1H), 7.75-7.60 (m, 2H), 7.55 (d, J = 9.6 Hz, 1H), 7.50 (s, 1H), 6.89 (dd, J = 2.4, 6.0 Hz, 1H), 5.22 (t, J = 5.2 Hz, 1H), 4.84 (t, J = 5.6 Hz, 1H), 4.78 (d, J = 12.8 Hz, 1H), 4.62-4.55 (m, 1H), 4.05-3.94 (m, 1H), 3.82-3.69 (m, 2H), 3.58-3.52 (m, 2H), 2.95-2.75 (m, 2H), 2.34 (s, 3H). |
| | (R)-7-(4-(3-fluoro-5- | | |
| | (trifluoromethyl)phenoxy)pyridin- | | |
| | 2-yl)-3-(2-hydroxyethyl)-5- | | |
| | (hydroxymethyl)-1-methyl-6,7- | | |
| | dihydroimidazo[1,5-α]pyrazin- | | |
| | 8(5H)-one | | |
| 344 | 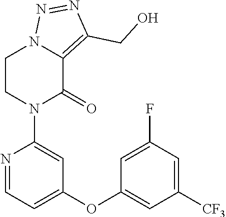 [SMILES: O=C1c2c(CO)nnn2CCN1c1cc(Oc2cc(F)cc(C(F)(F)F)c2)ccn1] | 424.0 | ¹H NMR (400 MHz, DMSO-d₆) δ = 8.45 (d, J = 5.6 Hz, 1H), 7.67 (d, J = 8.4 Hz, 1H), 7.61- 7.55 (m, 2H), 7.54-7.50 (m, 1H), 7.01 (dd, J = 2.4, 5.6 Hz, 1H), 5.20 (t, J = 6.0 Hz, 1H), 4.80-4.74 (m, 2H), 4.71 (d, J = 6.0 Hz, 2H), 4.59-4.50 (m, 2H). |
| | 5-(4-(3-fluoro-5- | | |
| | (trifluoromethyl)phenoxy)pyridin- | | |
| | 2-yl)-3-(hydroxymethyl)-6,7- | | |
| | dihydro-[1,2,3]triazolo[1,5- | | |
| | α]pyrazin-4(5H)-one | | |
| 346 | 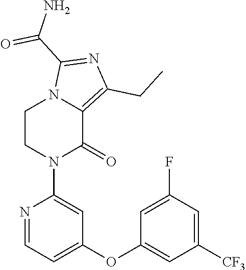 [SMILES: CCc1nc(C(N)=O)n2c1C(=O)N(c1cc(Oc3cc(F)cc(C(F)(F)F)c3)ccn1)CC2] | 464.2 | ¹H NMR (400 MHz, DMSO-d₆) δ = 8.41 (d, J = 5.6 Hz, 1H), 7.96 (s, 1H), 7.75 (s, 1H), 7.66- 7.64 (m, 2H), 7.56 (d, J = 9.2 Hz, 1H), 7.50 (s, 1H), 6.94 (dd, J = 2.4, 6.0 Hz, 1H), 4.73-4.70 (m, 2H), 4.39-4.36 (m, 2H), 2.85 (q, J = 7.2 Hz, 2H), 1.18 (t, J = 7.2 Hz, 3H). |
| | 1-ethyl-7-(4-(3-fluoro-5- | | |
| | (trifluoromethyl)phenoxy)pyridin- | | |
| | 2-yl)-8-oxo-5,6,7,8- | | |
| | tetrahydroimidazo[1,5- | | |
| | α]pyrazine-3-carboxamide | | |
| 347 | 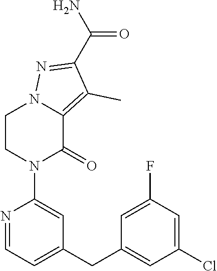 [SMILES: Cc1c(C(N)=O)nn2c1C(=O)N(c1cc(Cc3cc(F)cc(Cl)c3)ccn1)CC2] | 414.2 | ¹H NMR (400 MHz, CDCl₃) δ = 8.35 (d, J = 4.8 Hz, 1H), 7.94 (s, 1H), 7.00-6.97 (m, 2H), 6.92 (d, J = 4.8 Hz, 1H), 6.83-6.80 (m, 1H), 6.75 (s, 1H), 5.34 (s, 1H), 4.59-4.46 (m, 4H), 3.98 (s, 2H), 2.71 (s, 3H). |
| | 5-(4-(3-chloro-5- | | |
| | fluorobenzyl)pyridin-2-yl)-3- | | |
| | methyl-4-oxo-4,5,6,7- | | |
| | tetrahydropyrazolo[1,5- | | |
| | α]pyrazine-2-carboxamide | | |
| 348 | 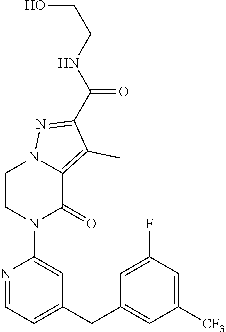 [SMILES: Cc1c(C(=O)NCCO)nn2c1C(=O)N(c1cc(Cc3cc(F)cc(C(F)(F)F)c3)ccn1)CC2] | 492.2 | ¹H NMR (400 MHz, DMSO-d₆) δ = 8.39 (d, J = 4.8 Hz, 1H), 8.03 (t, J = 6.0 Hz, 1H), 7.86 (s, 1H), 7.60 (s, 1H), 7.58-7.50 (m, 2H), 7.20 (d, J = 4.8 Hz, 1H), 4.81-4.70 (m, 1H), 4.52-4.46 (m, 2H), 4.46-4.40 (m, 2H), 4.16 (s, 2H), 3.48 (q, J = 6.0 Hz, 2H), 3.32-3.26 (m, 2H), 2.51 (br s, 3H). |
| | 5-(4-(3-fluoro-5- | | |
| | (trifluoromethyl)benzyl)pyridin- | | |
| | 2-yl)-N-(2-hydroxyethyl)-3- | | |
| | methyl-4-oxo-4,5,6,7- | | |
| | tetrahydropyrazolo[1,5- | | |
| | α]pyrazine-2-carboxamide | | |
| 349 | 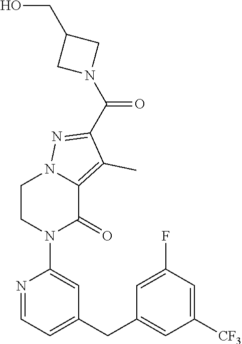 [SMILES: Cc1c(C(=O)N2CC(CO)C2)nn2c1C(=O)N(c1cc(Cc3cc(F)cc(C(F)(F)F)c3)ccn1)CC2] | 518.4 | ¹H NMR (400 MHz, DMSO-d₆) δ = 8.39 (d, J = 5.2 Hz, 1H), 7.86 (s, 1H), 7.60 (s, 1H), 7.58- 7.49 (m, 2H), 7.19 (d, J = 5.2 Hz, 1H), 4.83 (t, J = 5.2 Hz, 1H), 4.52-4.37 (m, 5H), 4.20- 4.09 (m, 3H), 4.05-3.94 (m, 1H), 3.83-3.70 (m, 1H), 3.54 (t, J = 5.6 Hz, 2H), 2.72-2.63 (m, 1H), 2.48 (s, 3H). |
| | 5-(4-(3-fluoro-5- | | |
| | (trifluoromethyl)benzyl)pyridin- | | |
| | 2-yl)-2-(3- | | |
| | (hydroxymethyl)azetidine-1- | | |
| | carbonyl)-3-methyl-6,7- | | |
| | dihydropyrazolo[1,5-α]pyrazin- | | |
| | 4(5H)-one | | |
| 350 | 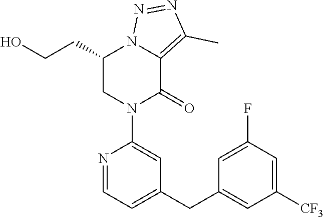 [SMILES: Cc1nnn2c1C(=O)N(c1cc(Cc3cc(F)cc(C(F)(F)F)c3)ccn1)C[C@@H]2CCO] | 450.1 | ¹H NMR (400 MHz, DMSO-d₆) δ = 8.40 (d, J = 5.2 Hz, 1H), 7.89 (s, 1H), 7.60 (s, 1H), 7.54 (t, J = 8.4 Hz, 2H), 7.21 (dd, J = 1.2, 5.2 Hz, 1H), 5.08-4.97 (m, 1H), 4.76 (t, J = 5.2 Hz, 1H), 4.60-4.52 (m, 1H), 4.49- 4.39 (m, 1H), 4.16 (s, 2H), 3.58 (q, J = 6.0 Hz, 2H), 2.48 (s, 3H), 2.31-2.19 (m, 1H), 2.03-1.91 (m, 1H). |
| | (S)-5-(4-(3-fluoro-5- | | |
| | (trifluoromethyl)benzyl)pyridin- | | |
| | 2-yl)-7-(2-hydroxyethyl)-3- | | |
| | methyl-6,7-dihydro- | | |
| | [1,2,3]triazolo[1,5-α]pyrazin- | | |
| | 4(5H)-one | | |
| 352 | 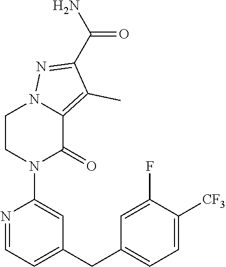 [SMILES: Cc1c(C(N)=O)nn2c1C(=O)N(c1cc(Cc3ccc(C(F)(F)F)c(F)c3)ccn1)CC2] | 448.2 | ¹H NMR (400 MHz, CDCl₃) δ = 8.35 (d, J = 5.2 Hz, 1H), 7.95 (s, 1H), 7.55 (t, J = 7.6 Hz, 1H), 7.11 (d, J = 7.6 Hz, 1H), 7.02 (d, J = 11.2 Hz, 1H), 6.91 (d, J = 4.4 Hz, 1H), 6.81-6.66 (m, 1H), 5.38-5.26 (m, 1H), 4.61- 4.54 (m, 2H), 4.49-4.42 (m, 2H), 4.05 (s, 2H), 2.69 (s, 3H). |
| | 5-(4-(3-fluoro-4- | | |
| | (trifluoromethyl)benzyl)pyridin- | | |
| | 2-yl)-3-methyl-4-oxo-4,5,6,7- | | |
| | tetrahydropyrazolo[1,5- | | |
| | α]pyrazine-2-carboxamide | | |
| 353 | 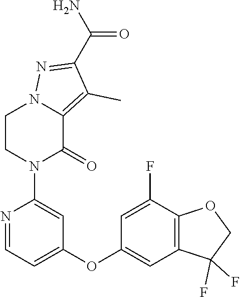 [SMILES: Cc1c(C(N)=O)nn2c1C(=O)N(c1cc(Oc3cc(F)c4c(c3)C(F)(F)CO4)ccn1)CC2] | 460.1 | ¹H NMR (400 MHz, DMSO-d₆) δ = 8.38 (d, J = 5.6 Hz, 1H), 7.60 (dd, J = 2.0, 11.2 Hz, 1H), 7.56-7.48 (m, 3H), 7.28 (s, 1H), 6.86 (dd, J = 2.0, 5.6 Hz, 1H), 5.01 (t, J = 16.4 Hz, 2H), 4.47 (s, 4H), 2.48 (s, 3H). |
| | 3-methyl-4-oxo-5-(4-((3,3,7- | | |
| | trifluoro-2,3-dihydrobenzofuran- | | |
| | 5-yl)oxy)pyridin-2-yl)-4,5,6,7- | | |
| | tetrahydropyrazolo[1,5- | | |
| | α]pyrazine-2-carboxamide | | |
| 354 | 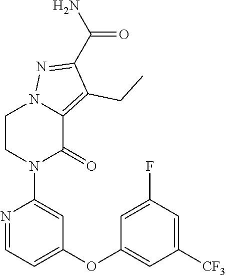 [SMILES: CCc1c(C(N)=O)nn2c1C(=O)N(c1cc(Oc3cc(F)cc(C(F)(F)F)c3)ccn1)CC2] | 464.1 | ¹H NMR (400 MHz, DMSO-d₆) δ = 8.44 (d, J = 5.6 Hz, 1H), 7.70-7.61 (m, 2H), 7.59-7.53 (m, 2H), 7.51 (s, 1H), 7.28 (s, 1H), 6.97 (dd, J = 2.4, 5.6 Hz, 1H), 4.49 (s, 4H), 3.03 (q, J = 7.2 Hz, 2H), 1.09 (t, J = 7.2 Hz, 3H). |
| | 3-ethyl-5-(4-(3-fluoro-5- | | |
| | (trifluoromethyl)phenoxy)pyridin- | | |
| | 2-yl)-4-oxo-4,5,6,7- | | |
| | tetrahydropyrazolo[1,5- | | |
| | α]pyrazine-2-carboxamide | | |
| 355 | 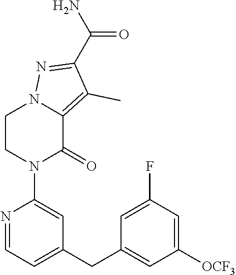 [SMILES: Cc1c(C(N)=O)nn2c1C(=O)N(c1cc(Cc3cc(F)cc(OC(F)(F)F)c3)ccn1)CC2] | 464.2 | ¹H NMR (400 MHz, DMSO-d₆) δ = 8.39 (d, J = 5.2 Hz, 1H), 7.85 (s, 1H), 7.55 (s, 1H), 7.31- 7.22 (m, 4H), 7.21-7.14 (m, 1H), 4.53-4.38 (m, 4H), 4.11 (s, 2H), 2.51 (s, 3H). |
| | 5-(4-(3-fluoro-5- | | |
| | (trifluoromethoxy)benzyl)pyridin- | | |
| | 2-yl)-3-methyl-4-oxo-4,5,6,7- | | |
| | tetrahydropyrazolo[1,5- | | |
| | α]pyrazine-2-carboxamide | | |
| 356 | 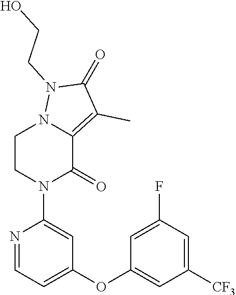 [SMILES: Cc1c2n(n(CCO)c1=O)CCN(c1cc(Oc3cc(F)cc(C(F)(F)F)c3)ccn1)C2=O] | 467.1 | ¹H NMR (400 MHz, CDCl₃) δ = 8.31 (d, J = 5.6 Hz, 1H), 7.79 (d, J = 2.0 Hz, 1H), 7.15 (d, J = 8.0 Hz, 1H), 7.12 (s, 1H), 6.97 (td, J = 2.4, 11.2 Hz, 1H), 6.70 (dd, J = 1.6, 5.6 Hz, 1H), 4.41 (t, J = 5.6 Hz, 2H), 3.95-3.90 (m, 2H), 3.89-3.84 (m, 2H), 3.61 (t, J = 5.6 Hz, 2H), 2.20 (s, 3H). |
| | 5-(4-(3-fluoro-5- | | |
| | (trifluoromethyl)phenoxy)pyridin- | | |
| | 2-yl)-1-(2-hydroxyethyl)-3- | | |
| | methyl-6,7-dihydropyrazolo[1,5- | | |
| | α]pyrazine-2,4(1H,5H)-dione | | |
| 358 | 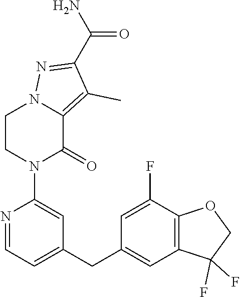 [SMILES: Cc1c(C(N)=O)nn2c1C(=O)N(c1cc(Cc3cc(F)c4c(c3)C(F)(F)CO4)ccn1)CC2] | 458.1 | ¹H NMR (400 MHz, DMSO-d₆) δ = 8.40-8.36 (m, 1H), 7.85- 7.82 (m, 1H), 7.58-7.53 (m, 1H), 7.53-7.47 (m, 1H), 7.46 (s, 1H), 7.34-7.24 (m, 1H), 7.21- 7.14 (m, 1H), 4.92 (t, J = 16.4 Hz, 2H), 4.52-4.39 (m, 4H), 4.06 (s, 2H), 2.51 (s, 3H). |
| | 3-methyl-4-oxo-5-(4-((3,3,7- | | |
| | trifluoro-2,3-dihydrobenzofuran- | | |
| | 5-yl)methyl)pyridin-2-yl)- | | |
| | 4,5,6,7-tetrahydropyrazolo[1,5- | | |
| | α]pyrazine-2-carboxamide | | |
| 359 | 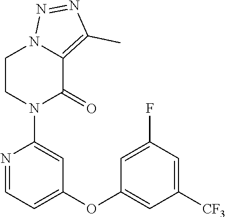 [SMILES: Cc1nnn2c1C(=O)N(c1cc(Oc3cc(F)cc(C(F)(F)F)c3)ccn1)CC2] | 408.0 | ¹H NMR (400 MHz, DMSO-d₆) δ = 8.44 (d, J = 5.6 Hz, 1H), 7.67 (d, J = 8.4 Hz, 1H), 7.60 (d, J = 2.0 Hz, 1H), 7.57 (d, J = 9.6 Hz, 1H), 7.51 (s, 1H), 6.99 (dd, J = 2.4, 5.6 Hz, 1H), 4.77- 4.69 (m, 2H), 4.59-4.49 (m, 2H), 2.45 (s, 3H). |
| | 5-(4-(3-fluoro-5- | | |
| | (trifluoromethyl)phenoxy)pyridin- | | |
| | 2-yl)-3-methyl-6,7-dihydro- | | |
| | [1,2,3]triazolo[1,5-α]pyrazin- | | |
| | 4(5H)-one | | |
| 360 | 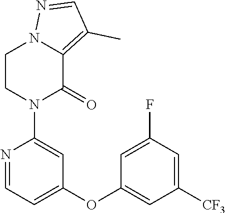 [SMILES: Cc1cnn2c1C(=O)N(c1cc(Oc3cc(F)cc(C(F)(F)F)c3)ccn1)CC2] | 407.0 | ¹H NMR (400 MHz, DMSO-d₆) δ = 8.43 (d, J = 5.6 Hz, 1H), 7.67 (d, J = 8.4 Hz, 1H), 7.62 (d, J = 2.0 Hz, 1H), 7.59-7.54 (m, 1H), 7.51 (s, 1H), 7.46 (s, 1H), 6.96 (dd, J = 2.0, 5.6 Hz, 1H), 4.44 (d, J = 2.0 Hz, 4H), 2.24 (s, 3H). |
| | 5-(4-(3-fluoro-5- | | |
| | (trifluoromethyl)phenoxy)pyridin- | | |
| | 2-yl)-3-methyl-6,7- | | |
| | dihydropyrazolo[1,5-α]pyrazin- | | |
| | 4(5H)-one | | |
| 361 | 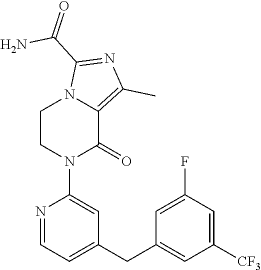 [SMILES: Cc1nc(C(N)=O)n2c1C(=O)N(c1cc(Cc3cc(F)cc(C(F)(F)F)c3)ccn1)CC2] | 448.1 | ¹H NMR (400 MHz, DMSO-d₆) δ = 8.36 (d, J = 5.2 Hz, 1H), 8.01 (s, 1H), 7.87 (s, 1H), 7.73 (s, 1H), 7.62-7.50 (m, 3H), 7.16 (dd, J = 1.2, 5.2 Hz, 1H), 4.71 (dd, J = 5.2, 7.2 Hz, 2H), 4.36- 4.27 (m, 2H), 4.15 (s, 2H), 2.45 (s, 3H). |
| | 7-(4-(3-fluoro-5- | | |
| | (trifluoromethyl)benzyl)pyridin- | | |
| | 2-yl)-1-methyl-8-oxo-5,6,7,8- | | |
| | tetrahydroimidazo[1,5- | | |
| | α]pyrazine-3-carboxamide | | |
| 362 | 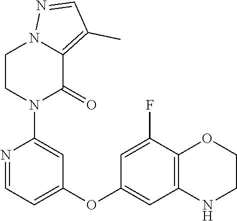 [SMILES: Cc1cnn2c1C(=O)N(c1cc(Oc3cc(F)c4c(c3)NCCO4)ccn1)CC2] | 396.1 | ¹H NMR (400 MHz, DMSO-d₆) δ = 8.33 (d, J = 5.6 Hz, 1H), 7.51-7.42 (m, 2H), 6.81 (dd, J = 2.4, 5.6 Hz, 1H), 6.40 (s, 1H), 6.29 (dd, J = 2.8, 11.2 Hz, 1H), 6.22-6.17 (m, 1H), 4.41 (s, 4H), 4.16 (t, J = 4.0 Hz, 2H), 3.37- 3.33 (m, 2H), 2.25 (s, 3H). |
| | 5-(4-((8-fluoro-3,4-dihydro-2H- | | |
| | benzo[b][1,4]oxazin-6- | | |
| | yl)oxy)pyridin-2-yl)-3-methyl- | | |
| | 6,7-dihydropyrazolo[1,5- | | |
| | α]pyrazin-4(5H)-one | | |
| 363 | 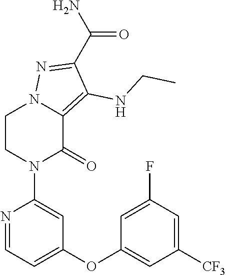 [SMILES: CCNc1c(C(N)=O)nn2c1C(=O)N(c1cc(Oc3cc(F)cc(C(F)(F)F)c3)ccn1)CC2] | 479.1 | ¹H NMR (400 MHz, DMSO-d₆) δ = 8.41 (d, J = 5.6 Hz, 1H), 7.65 (d, J = 8.4 Hz, 1H), 7.60 (d, J = 2.4 Hz, 1H), 7.59-7.52 (m, 2H), 7.51 (s, 1H), 7.28 (s, 1H), 6.93 (dd, J = 2.4, 5.6 Hz, 1H), 6.23 (t, J = 6.4 Hz, 1H), 4.47-4.36 (m, 4H), 3.45-3.35 (m, 2H), 1.03 (t, J = 7.2 Hz, 3H). |
| | 3-(ethylamino)-5-(4-(3-fluoro-5- | | |
| | (trifluoromethyl)phenoxy)pyridin- | | |
| | 2-yl)-4-oxo-4,5,6,7- | | |
| | tetrahydropyrazolo[1,5- | | |
| | α]pyrazine-2-carboxamide | | |

### D. BIOLOGICAL ACTIVITY

### Example A. Functional cAMP Assay

cAMP accumulation was measured using a high-throughout, ultra-sensitive homogeneous time-resolved FRET assay (HTRF) under the conditions below. The assay was established in-house based on the LANCE Ultra CAMP kit (PerkinElmer). The suspension protocol was optimised for GPR52 expressed in the FlpIn-TREx-HEK293 inducible system. HEK293-TREx FLAG-hGPR52 cells were maintained in DMEM supplemented with 5% FBS and 200 μg/mL Hygromycin B in T150 cm² flasks. One day prior to the assay, cells were split into a T75 cm² flask and topped up to 8 mL using DMEM (5% FBS with Hygromycin B) plus 300 ng/mL tetracycline (Sigma) to induce GPR52 expression. Cells were incubated at 37° C., supplemented with 5% CO₂ for 16-18h. On the day of the assay, induced cells were detached and counted. The concentration was adjusted to 60 cells/μL in cAMP assay stimulation buffer (1×HBSS (Thermofisher) with 20 μM rolipram (Tocris Bioscience), 5 mM HEPES, and 0.1% BSA and equilibrated for 30 min at room temperature. Compounds were serially diluted in a 96 w V-bottom plate for a 2× working solution. An 11-point half logarithmic serial dilution was performed in assay buffer+0.63% DMSO to obtain a final concentration range of 31.6 μM (10^{-4.5}) to 0.316 nM (10-9.5). 3-BTBZ (compound 7m in Setoh et al, J. Med. Chem. 2014, 57, 5226-5237) was used as positive control and was serially diluted in the same manner as test compounds above. The final concentration of DMSO in each well is kept consistent at 0.316%, 5 μL of the 2× serially diluted compounds, control and 5 μL of the diluted cell suspension (300 cells) were transferred into each well of a 384-well Optiplate. The plate was incubated at room temperature for 30 min. The reaction was terminated by adding 5 μL of lysis buffer containing Eu-CAMP and ULight antibody which were diluted according to the CAMP kit instructions. The TR-FRET signal at 665 nm was measured using a PerkinElmer Envision plate reader with an excitation wavelength of 320 nm and an emission wavelength of 665 nm. Absolute cAMP levels for each compound were determined by interpolating raw RFU values from a standard curve. Raw CAMP values were normalised at 0% to vehicle control, and at 100% to the top of 3-BTBZ response. Potency and Emax values (Table 5) were obtained from GraphPad Prism by fitting to a 3-parameter Hill equation.

TABLE 5
cAMP Accumulation

cAMP data

| Cmpd. | | pEC₅₀ | Eₘₐₓ | Eₘₐₓ |
|---|---|---|---|---|
| No. | pEC₅₀ | SEM | (%) | SEM |
| 1 | 5.7 | 0.1 | 93 | 4 |
| 2 | 8.1 | 0.1 | 106 | 3 |
| 3 | 6.5 | 0.1 | 92 | 3 |
| 4 | 6.2 | 0.1 | 99 | 3 |
| 5 | 6.9 | 0.1 | 83 | 3 |
| 6 | 6.4 | 0.2 | 97 | 8 |
| 7 | 4.9 | 0.3 | 75 | 18 |
| 9 | 7.4 | 0.1 | 101 | 4 |
| 10 | 7.3 | 0.1 | 94 | 3 |
| 11 | 7.1 | 0.1 | 87 | 2 |
| 12 | 6.3 | 0.1 | 87 | 3 |
| 13 | 7.9 | 0.1 | 80 | 2 |
| 14 | 8.1 | 0.1 | 98 | 2 |
| 15 | 6.8 | 0.1 | 92 | 3 |
| 16 | 8.1 | 0.1 | 87 | 3 |
| 17 | 6.7 | 0.1 | 73 | 2 |
| 18 | 7.7 | 0.1 | 120 | 4 |
| 19 | 6.4 | 0.1 | 86 | 3 |
| 20 | 5.5 | 0.1 | 88 | 5 |
| 21 | 6.5 | 0.1 | 32 | 2 |
| 22 | 6.2 | 0.1 | 56 | 3 |
| 23 | 7.4 | 0.1 | 111 | 4 |
| 24 | 6.3 | 0.1 | 72 | 4 |
| 25 | 5.4 | 0.1 | 96 | 5 |
| 26 | 7.7 | 0.1 | 97 | 2 |
| 27 | 7.5 | 0.1 | 103 | 3 |
| 28 | 7.3 | 0.1 | 83 | 2 |
| 29 | 6.4 | 0.1 | 79 | 3 |
| 30 | 6 | 0 | 76 | 2 |
| 31 | 7.2 | 0.1 | 99 | 3 |
| 32 | 5.9 | 0.1 | 101 | 4 |
| 33 | 8.1 | 0.1 | 110 | 3 |
| 34 | 7.5 | 0.1 | 88 | 3 |
| 35 | 7.3 | 0.1 | 110 | 2 |
| 36 | 6.9 | 0.1 | 100 | 3 |
| 37 | 6.6 | 0.1 | 78 | 3 |
| 38 | 7.2 | 0.1 | 101 | 2 |
| 39 | 7.4 | 0.1 | 95 | 3 |
| 40 | 7.3 | 0.1 | 85 | 2 |
| 41 | 7.1 | 0.1 | 104 | 3 |
| 42 | 6.9 | 0.1 | 97 | 3 |
| 43 | 5.7 | 0.1 | 87 | 5 |
| 44 | 7.2 | 0.1 | 79 | 2 |
| 45 | 5.2 | 0.1 | 94 | 9 |
| 46 | 5.1 | 0.2 | 79 | 11 |
| 47 | 6.1 | 0.1 | 83 | 2 |
| 48 | 6.8 | 0.1 | 86 | 2 |
| 49 | 7.1 | 0.1 | 78 | 3 |
| 50 | 7.9 | 0.1 | 101 | 2 |
| 51 | 7.4 | 0.1 | 99 | 3 |
| 52 | 7.4 | 0.1 | 98 | 3 |
| 53 | 6.6 | 0.1 | 90 | 3 |
| 54 | 7.2 | 0.1 | 99 | 3 |
| 55 | 7.6 | 0 | 96 | 1 |
| 56 | 6.9 | 0 | 101 | 2 |
| 57 | 7.7 | 0.1 | 88 | 2 |
| 58 | 6.7 | 0 | 97 | 2 |
| 59 | 6.8 | 0.1 | 97 | 2 |
| 60 | 6 | 0 | 92 | 2 |
| 61 | 6.1 | 0.1 | 64 | 3 |
| 62* | 7 | 0.1 | 85 | 4 |
| 63* | 8.1 | 0.1 | 77 | 2 |
| 64* | 7.2 | 0.1 | 97 | 3 |
| 65* | 6.8 | 0.1 | 94 | 3 |
| 66* | 6.6 | 0.1 | 81 | 3 |
| 67* | 7.1 | 0.1 | 76 | 2 |
| 68* | 7.3 | 0.1 | 85 | 3 |
| 69 | 4.5 | 0.1 | 127 | 23 |
| 70 | 7.4 | 0.1 | 87 | 3 |
| 71 | 8.1 | 0.1 | 96 | 3 |
| 72 | 7.7 | 0.1 | 116 | 5 |
| 73a/73b | 6.9 | 0.1 | 89 | 3 |
| 74 | 6.5 | 0 | 88 | 1 |
| 75 | 4.8 | 0.2 | 107 | 25 |
| 76 | 5.8 | 0.1 | 93 | 4 |
| 77 | 6.9 | 0.1 | 76 | 2 |
| 78 | 5.9 | 0.1 | 100 | 3 |
| 79 | 6.6 | 0.1 | 81 | 2 |
| 80 | 6.5 | 0.1 | 95 | 3 |
| 81 | 7.2 | 0.1 | 101 | 2 |
| 82 | 7 | 0.1 | 100 | 4 |
| 83 | 5.7 | 0.3 | 82 | 12 |
| 84 | 5.8 | 0.1 | 128 | 7 |
| 3-BTBZ | 6.8 | 0.1 | 96 | 3 |
| E39 | 7.2 | 0.1 | 95 | 2 |

*n = 2

Alternatively, the ability of a compound to increase accumulation of cAMP produced upon modulation of adenylyl cyclase activity by GPR52 was measured using the TR-FRET immunoassay LANCE Ultra CAMP Detection kit obtained from Revvity (#TRF0264). HBSS+20 mM HEPES+500 μM Rolipram (20 μM)+0.1% BSA, pH7.4 was used as assay buffer. Compounds were serially diluted for 10 points in duplicate in DMSO by ECHO Liquid Handler and spot 50 nL to assay plate following the plate layout below. 50 nL of reference compound (4-(3-(3-fluoro-5-(trifluoromethyl)benzyl)-5-methyl-1H-1,2,4-triazol-1-yl)-2-methylbenzamide) was transferred to High control wells. The final maximum concentration of compounds tested was 10 μM. The final DMSO content was 0.5%.

Stable cells were archived as frozen aliquots in liquid nitrogen. Cells were thawed and expanded according to ATCC protocols. Cells were not used in the assays past 15 rounds of passage or if any shift in the assay controls were observed.

The GPR52 stable cells were digested with 0.25% trypsin and collected the cell by centrifugation at 1000 rpm for 5 min. The cells were washed twice with HBSS and collected by centrifugation at 1000 rpm for 5 min. Then, the cells were resuspended in assay buffer and counted via cell counter. The cell density was adjusted with assay buffer to 50000/mL and 10 μL of this cell suspension (500 cells/well) was added to each well of the assay plate as the plate map. The cells and compounds were mixed well and incubated at RT for 1 hour. A CAMP standard curve was prepared. Starting at 10 μM, 4-fold serial dilutions with assay buffer were preformed of cAMP standard to generate 10 points. 10 μL of each concentration solution was transferred to the assay plate. CisBio detection reagents were prepared (Eu labeled antibody+cAMP tracer) according to the the CAMP detection kit manual and 10 μL of detection reagent was added to each well in the assay plate. Then, the assay plate was incubated at RT for 1 hour. The plate was subsequently read by Envision (665 nm/615 nm). 5

An HTRF ratio (665 nm/615 nm) was used directly to calculate the percent effect using the following equation: % Effect=(sample well HTRF ratio−Average HTRF ratio of Low controls)/(Average HTRF ratio of High controls−Average HTRF ratio of Low controls)*100%. The Dose-Response curve was fit using Prism 5 using the model “log (agonist) vs. response--Variable slope” to calculate the EC₅₀. The CAMP concentration of 10 the sample wells was calculated using the HTRF ratio (665 nm/615 nm) according to the standard curve and ensure output values fall on the linear part of the curve. The data is summarized in Table 6 below.

TABLE 6
cAMP Accumulation

| | HEK293 cAMP |
|---|---|
| | HTRF EC₅₀ |
| Compound | (nM) |
| 85 | 2.1 |
| 86 | 58.4 |
| 87 | 44.6 |
| 88 | 97.7 |
| 89 | 42.9 |
| 90 | 5.3 |
| 91 | 264 |
| 92 | 32.1 |
| 93 | 3 |
| 94 | 88.9 |
| 95 | 3.6 |
| 96 | 0.8 |
| 97 | 27.8 |
| 98 | 14.9 |
| 99 | 64.8 |
| 100 | 39.6 |
| 101 | 689 |
| 102 | 1460 |
| 103 | 0.9 |
| 104 | 9.5 |
| 105 | 3.4 |
| 106 | 8.0 |
| 107 | 1.3 |
| 108 | 0.4 |
| 109 | 1.6 |
| 110 | 3.4 |
| 111 | 265 |
| 112 | 1.8 |
| 113 | 1.2 |
| 114 | 104 |
| 115 | >674 |
| 116 | 55.5 |
| 117 | >387 |
| 118 | 73.6 |
| 119 | 172 |
| 120 | 49.7 |
| 121 | 60.1 |
| 122 | 25.3 |
| 123 | 1.6 |
| 124 | >544 |
| 125 | 39.1 |
| 126 | 128 |
| 127 | 450 |
| 128 | 9.1 |
| 129 | 2.5 |
| 130 | 7.0 |
| 131 | 47.6 |
| 132 | 81.2 |
| 133 | 7.7 |
| 134 | 12.8 |
| 135 | 4.9 |
| 136 | 6.2 |
| 137 | 2.7 |
| 138 | 40.2 |
| 139 | 1.6 |
| 140 | 11.6 |
| 141 | 1.0 |
| 142 | 2.9 |
| 143 | 2.8 |
| 144 | 3.1 |
| 145 | 2.0 |
| 146 | 1.5 |
| 147 | 1.9 |
| 148 | 3.9 |
| 149 | 3.9 |
| 150 | 45.5 |
| 151 | 87.3 |
| 152 | 50.8 |
| 153 | 4.5 |
| 154 | 4.4 |
| 155 | 1.9 |
| 156 | 4.7 |
| 157 | 7 |
| 158 | 36.5 |
| 159 | 10.1 |
| 160 | 1.2 |
| 161 | 8.3 |
| 162 | 2.1 |
| 163 | 40.5 |
| 164 | 32.2 |
| 165 | 2.4 |
| 166 | 23.6 |
| 167 | 17.9 |
| 168 | 6.2 |
| 169 | 17.4 |
| 170 | 19.2 |
| 171 | 10.2 |
| 172 | 22.7 |
| 173 | 51.9 |
| 174 | 4.5 |
| 175 | 5.1 |
| 176 | 7.1 |
| 177 | 26.8 |
| 178 | 2.1 |
| 179 | 6.3 |
| 180 | 12.2 |
| 181 | 2.6 |
| 182 | 7.4 |
| 183 | 19.1 |
| 184 | 27.2 |
| 185 | 23.5 |
| 186 | 19.0 |
| 187 | 8.7 |
| 188 | 24.2 |
| 189 | 11.5 |
| 190 | 47.9 |
| 191 | 25.5 |
| 192 | 34 |
| 193 | 3.1 |
| 194 | 5.1 |
| 195 | 3.6 |
| 196 | 13 |
| 197 | 1.1 |
| 198 | 0.6 |
| 199 | 0.9 |
| 200 | 1.1 |
| 201 | 169 |
| 202 | 211 |
| 203 | 1.4 |
| 204 | 4.9 |
| 205 | 10.6 |
| 206 | 6.6 |
| 207 | 5.0 |
| 208 | >1000 |
| 209 | 6.9 |
| 210 | 6.0 |
| 211 | 30.4 |
| 212 | 294 |
| 213 | 2.3 |
| 214 | 16.4 |
| 215 | 0.5 |
| 216 | 1.0 |
| 217 | 23.6 |
| 218 | 4.5 |
| 219 | 16.1 |
| 220 | 4.0 |
| 221 | 1.5 |
| 222 | 1.0 |
| 223 | 0.9 |
| 224 | 2.0 |
| 225 | 52.7 |
| 226 | 6.8 |
| 227 | 7.0 |
| 228 | 2.9 |
| 229 | 1.3 |
| 230 | 0.7 |
| 231 | 32.3 |
| 232 | >428 |
| 233 | 5.5 |
| 234 | 2.6 |
| 235 | 4.3 |
| 236 | 5.1 |
| 237 | 3.5 |
| 238 | 13.8 |
| 239 | 31.1 |
| 240 | 11.1 |
| 241 | 6.1 |
| 242 | 14.9 |
| 243 | 37.8 |
| 244 | 1.9 |
| 245 | 2.6 |
| 246 | 12.8 |
| 247 | 16.5 |
| 248 | 15.3 |
| 249 | 4.3 |
| 250 | 6.3 |
| 251 | 2 |
| 252 | 0.8 |
| 253 | 45.1 |
| 254 | 31 |
| 255 | 0.9 |
| 256 | 8.3 |
| 257 | 10.5 |
| 258 | 2.5 |
| 259 | 0.7 |
| 260 | 1.3 |
| 261 | 1.3 |
| 262 | 16.4 |
| 263 | 1.0 |
| 264 | 605 |
| 265 | 106 |
| 266 | 28.2 |
| 267 | 2.5 |
| 268 | 21.5 |
| 269 | 2.2 |
| 270 | 5.2 |
| 271 | 1.7 |
| 272 | 7.0 |
| 273 | 192 |
| 274 | 31.3 |
| 275 | 305 |
| 276 | 13.1 |
| 277 | 6.5 |
| 278 | 4.8 |
| 279 | 2.4 |
| 280 | 5.1 |
| 281 | 4.9 |
| 282 | 13.7 |
| 283 | 3.7 |
| 284 | 8.0 |
| 285 | >435 |
| 286 | 5.3 |
| 287 | 20.7 |
| 288 | 7.5 |
| 289 | 3.1 |
| 290 | 6.5 |
| 291 | 1.5 |
| 292 | 10.4 |
| 293 | 5.3 |
| 294 | 11.5 |
| 295 | 2.7 |
| 296 | 347 |
| 297 | 2.7 |
| 298 | 2.8 |
| 299 | >292 |
| 300 | 17.5 |
| 301 | 1.6 |
| 302 | 5.2 |
| 303 | 2.6 |
| 304 | 3.1 |
| 305 | 112 |
| 306 | >192 |
| 307 | 3.7 |
| 308 | 19.4 |
| 309 | 4.1 |
| 310 | 20.8 |
| 311 | 332 |
| 312 | 1.2 |
| 314 | 2.9 |
| 315 | 1.1 |
| 317 | 142 |
| 318 | >1000 |
| 319 | 3.3 |
| 320 | 70.3 |
| 321 | 1.8 |
| 322 | 16.3 |
| 323 | 232 |
| 324 | 3.6 |
| 325 | 18.2 |
| 326 | 0.25 |
| 327 | 0.72 |
| 328 | >1000 |
| 329 | 43.6 |
| 330 | 109 |
| 331 | 0.78 |
| 332 | 5.4 |
| 333 | 22.9 |
| 334 | 7 |
| 335 | >334 |
| 336 | 67.6 |
| 337 | 9.8 |
| 338 | 0.16 |
| 339 | 32 |
| 340 | 22 |
| 341 | 123 |
| 342 | 57.1 |
| 343 | 127 |
| 344 | >451 |
| 345 | 44.1 |
| 346 | 13.2 |
| 347 | 1.6 |
| 348 | 2.3 |
| 349 | 6.2 |
| 350 | 2.0 |
| 351 | 0.86 |
| 352 | 9.0 |
| 353 | 1.9 |
| 354 | 2.9 |
| 355 | 0.76 |
| 356 | 6.2 |
| 357 | 51.3 |
| 358 | 1.2 |
| 359 | 14.8 |
| 360 | 51.8 |
| 361 | 1.1 |
| 362 | 4.6 |
| 363 | 3.2 |
| 364 | 16 |
| 365 | 92.9 |
| 366 | 1.0 |
| 367 | 0.94 |

### Example B. Beta-Arrestin Assay

A bioluminescence energy transfer (BRET) assay was used to monitor β-arrestin-2 recruitment to GPR52. Prior to transfection, HEK293A cells must be at ~80-90% confluency in a T150 flask. Media was removed and cells were detached with Versene followed by 4 minutes of centrifugation at 350 g to obtain a cell pellet. Cell pellets were resuspended in 10 mL DMEM+5% FBS and counted on the haemocytometer with Trypan blue. 8 million cells were seeded in a T150 flask to reach ~80% confluency the following day. Cells are incubated for 24 hr at 37° C., supplemented with 5% CO₂. On day 2, one tube was prepared with 100 μL LTX reagent and 400 μL OptiMEM and left for 5 min. In a second tube, 10 μg GPR52-Nluc DNA, 10 μg β-Arrestin-2-eYFP DNA and 20 μL PLUS reagent were added to 500 μL OptiMEM and left for 5 min. The transfection complexes were combined to make 1 mL total volume, and gently mixed by inversion, before incubating for 30 min to allow formation of DNA-lipid complex. Following incubation, the DNA-lipid complex was slowly added into the DMEM medium in the flask and further incubated overnight for transfection.

Cells were assessed for successful transfection using the ZOE Fluorescent Cell Imager (BIO-RAD) via observation of yellow fluorescent protein (YFP) inside the cytoplasm. Transfected cells were detached using 2 μM EDTA in 1×PBS and centrifuged at 350 g for 4 min to obtain the cell pellet. The pellet was gently resuspended 5 times with 1 mL Phenol Red Free DMEM (+5% FBS) before topping up with 9 mL Phenol Red Free DMEM and further resuspending. Cells were counted and the volume was adjusted 300,000 cells/mL.

The first 6 columns of a white bottom 96-well CulturPlate were coated with 50 μL/well (50 μg/mL) poly-D-lysine and left for 1 hr. The plate was washed once with water 1× PBS and air dried prior to use. 100 μL was added per well to obtain 30,000 cells per well. In a clear-bottom regular 96-well plate, the same amount was seeded into 2-3 wells as a test plate. Plates were incubated overnight to allow cell attachment.

30 min before each individual plate's run, the medium was replaced with 80 μL pre-warmed assay buffer (1×HBSS, 5 μM HEPES 0.1% and adjusted to pH 7.4) A 10× compound plate containing novel compounds of the current invention, vehicle control and 3-BTBZ control compound was prepared using the assay buffer. The LUMIstar plate reader (BMG Labtech) was set to 37° C. NanoBRET NanoGlo substrate (Promega Corp., 1:500) was prepared immediately prior to each run, and 10 μL was added per well and incubated for 5 min prior to start of data acquisition at 1s per well. 4 cycles of baseline measurement are obtained, and 10 μL of test compounds (10×) were added prior to the 5th cycle.

Dual wavelengths were recorded at 475 nm (GPR52-Nluc) and 535 nm (β-Arrestin-2-eYFP). The BRET ratio was calculated as a signal at 535 nm/475 nm using the BMG MARS software. AABRET values were normalised using GraphPad Prism by subtracting the baseline and vehicle BRET responses. Average AABRET values were used to create a concentration response curve and calculate pEC₅₀ and Eₘₐₓ values (Table 7).

TABLE 7
GPR52 pEC₅₀ data

β-Arr2 data

| Cmpd. | | pEC₅₀ | Eₘₐₓ | Eₘₐₓ |
|---|---|---|---|---|
| No. | pEC₅₀ | SEM | (%) | SEM |
| 2 | 7.2 | 0.2 | 79 | 6 |
| 5 | 6 | 0.1 | 68 | 5 |
| 10 | 6.4 | 0.1 | 81 | 6 |
| 11 | 6.2 | 0.2 | 101 | 9 |
| 13 | 7.1 | 0.3 | 72 | 7 |
| 14 | 7.1 | 0.2 | 82 | 6 |
| 15 | 6.1 | 0.2 | 74 | 8 |
| 16 | 7.7 | 0.2 | 80 | 5 |
| 17 | 6.3 | 0.3 | 70 | 11 |
| 18 | 7 | 0.2 | 81 | 4 |
| 23 | 6.6 | 0.1 | 72 | 5 |
| 26 | 7.2 | 0.2 | 67 | 5 |
| 27 | 7 | 0.2 | 42 | 4 |
| 28 | 6.7 | 0.3 | 80 | 9 |
| 31 | 6.4 | 0.2 | 75 | 7 |
| 33 | 7.6 | 0.1 | 100 | 4 |
| 34 | 6.7 | 0.2 | 81 | 6 |
| 35 | 6.8 | 0.1 | 98 | 4 |
| 36 | 6.1 | 0.1 | 88 | 7 |
| 38 | 7.1 | 0.1 | 93 | 4 |
| 39 | 7.6 | 0.2 | 92 | 6 |
| 40 | 6.8 | 0.2 | 75 | 6 |
| 41 | 6.9 | 0.1 | 78 | 4 |
| 42 | 6.7 | 0.2 | 79 | 6 |
| 44 | 6.5 | 0.2 | 78 | 7 |
| 47 | <5 | | | |
| 48 | 6 | 0.1 | 85 | 6 |
| 49 | 6.5 | 0.1 | 76 | 5 |
| 50 | 7 | 0.1 | 90 | 4 |
| 70 | 7 | 0.2 | 78 | 5 |
| 71 | 7.7 | 0.2 | 72 | 4 |
| 72 | 7.6 | 0.1 | 83 | 4 |
| 73a/73b | 6.6 | 0.2 | 63 | 5 |
| 77 | 6.2 | 0.1 | 72 | 4 |
| 78 | <5.0 | | | |
| 79 | 6.2 | 0.1 | 78 | 6 |
| 3-BTBZ | 6.7 | 0.0 | 97 | 2 |
| E39 | 6.3 | 0.1 | 86 | 4 |

Alternatively, the ability of a compound to increase the recruitment of β-arrestin-2 to GPR52 upon binding was measured using the NanoBiT luciferase assay obtained from Promega. HEK293A cells were grown to 90% confluency in a T150 flask. Media was removed and cells were detached with 2 mM EDTA in 1×PBS followed by 4 minutes of centrifugation at 350 g to obtain a cell pellet. The cell pellet was resuspended in 10 mL DMEM+5% FBS and counted on the hemocytometer with Trypan blue. 8 million cells were seeded in a T150 flask to reach ~80% confluency the following day. Cells are incubated for 24 hr at 37° C., supplemented with 5% CO₂.

Stable cells were archived as frozen aliquots in liquid nitrogen. Cells were thawed and expanded according to ATCC protocols. Cells were not used in the assays past 15 rounds of passage or if any shift in the assay controls was observed.

The GPR52 stable cells were digested with 0.25% trypsin and collected by centrifugation at 1000 rpm for 5 min. The cells were washed twice with HBSS and collected by centrifugation at 1000 rpm for 5 min. The cells were resuspended with assay buffer and counted via cell counter. 30k cells were plated in 40 ul/well in a 384 well plate and incubated overnight at 37° C., supplemented with 5% CO₂. Compounds were dissolved and diluted with ECHO Liquid Handler in the compound plate according to the plate map. Compounds were then diluted further by 10× into phenol red-free opti MEM. 5 μL of compound solutions (10×) was mixed with 5 μL of detection reagent (refer to Promega protocol) and added to the cell plate. Measurements were acquired according to Promega protocol by Envision 30 minutes after compound additions and incubation.

The luciferase ratio was used directly to calculate the percent effect using the following equation: % Effect=(sample well luc ratio-Average luc ratio of Low controls)/(Average luc ratio of High controls−Average luc ratio of Low controls)*100%. The Dose-Response curve was fit using Prism 5. Use the model “log (agonist) vs. response--Variable slope” to calculate the EC₅₀. The results are shown in Tables below. Key: N.D.=not determined.

TABLE 8
beta Arrestin

| | HEK293A β-arrestin-2 |
|---|---|
| Compound | luciferase EC₅₀ (nM) |
| 85 | 32.1 |
| 86 | 130 |
| 87 | 441 |
| 89 | 269 |
| 90 | 187 |
| 91 | 5520 |
| 93 | 38.9 |
| 94 | 8620 |
| 95 | 10.2 |
| 96 | 45.5 |
| 97 | 40.6 |
| 98 | 262 |
| 99 | 1430 |
| 100 | >10000 |
| 103 | 11.8 |
| 104 | 111 |
| 105 | 7.97 |
| 106 | 8480 |
| 107 | 21.1 |
| 108 | 8.56 |
| 109 | 61.2 |
| 110 | 76.3 |
| 111 | 1920 |
| 112 | 17.1 |
| 113 | 11.3 |
| 114 | >5090 |
| 115 | >10000 |
| 116 | 2760 |
| 117 | 3600 |
| 118 | 1680 |
| 119 | >10000 |
| 120 | 1170 |
| 121 | 4630 |
| 122 | 1590 |
| 123 | 42.3 |
| 124 | 3440 |
| 125 | 747 |
| 126 | 3300 |
| 127 | >5990 |
| 128 | 302 |
| 129 | 79.7 |
| 130 | 121 |
| 131 | 970 |
| 132 | 1870 |
| 133 | 97.1 |
| 134 | 472 |
| 135 | 81.3 |
| 136 | 88.6 |
| 137 | 42.1 |
| 138 | 852 |
| 139 | 23.3 |
| 140 | 135 |
| 141 | 9.86 |
| 142 | 15.3 |
| 143 | 63.7 |
| 144 | 94.5 |
| 145 | 56.8 |
| 146 | 36 |
| 147 | 17.2 |
| 148 | 96.6 |
| 149 | 1210 |
| 150 | 943 |
| 151 | 1810 |
| 152 | 1230 |
| 153 | 83.8 |
| 154 | 17.2 |
| 155 | 45.5 |
| 156 | 135 |
| 157 | >482 |
| 158 | 830 |
| 159 | 173 |
| 160 | 23.2 |
| 161 | 128 |
| 162 | 30.5 |
| 163 | 573 |
| 164 | 902 |
| 165 | 51.5 |
| 166 | 904 |
| 167 | 509 |
| 168 | 202 |
| 169 | >808 |
| 170 | 443 |
| 171 | 378 |
| 172 | >10000 |
| 173 | >10000 |
| 174 | 53.7 |
| 175 | 110 |
| 176 | 110 |
| 177 | 453 |
| 178 | 14 |
| 179 | 206 |
| 180 | 202 |
| 181 | 80 |
| 182 | 189 |
| 183 | 671 |
| 184 | 1360 |
| 185 | >10000 |
| 186 | >10000 |
| 187 | >1830 |
| 188 | 2260 |
| 189 | 176 |
| 190 | >2690 |
| 191 | >3220 |
| 192 | >3710 |
| 193 | 55.1 |
| 194 | 154 |
| 195 | 82.3 |
| 196 | >223 |
| 197 | <5.13 |
| 198 | 5.01 |
| 199 | 25.1 |
| 200 | 30.4 |
| 201 | 3840 |
| 202 | >10000 |
| 203 | 20.5 |
| 204 | 135 |
| 205 | 417 |
| 206 | 163 |
| 207 | >10000 |
| 208 | 5180 |
| 209 | 217 |
| 210 | 94.2 |
| 211 | 719 |
| 212 | 3260 |
| 213 | 20.6 |
| 214 | 659 |
| 215 | 5.97 |
| 216 | 78.5 |
| 217 | 429 |
| 218 | >162 |
| 219 | 86 |
| 220 | 143 |
| 221 | 47.6 |
| 222 | 9.58 |
| 223 | 7.56 |
| 224 | 21.9 |
| 225 | 2740 |
| 226 | 13 |
| 227 | 83.7 |
| 228 | 70.0 |
| 229 | 21.7 |
| 230 | 15.3 |
| 231 | 524 |
| 232 | 2170 |
| 233 | 97.2 |
| 234 | 56.2 |
| 235 | 122 |
| 236 | 234 |
| 237 | 52.3 |
| 238 | 358 |
| 239 | 612 |
| 240 | 213 |
| 241 | 272 |
| 242 | 506 |
| 243 | 406 |
| 244 | 28.8 |
| 245 | 70.8 |
| 246 | 109 |
| 247 | 174 |
| 248 | 270 |
| 249 | 52.9 |
| 250 | 85.4 |
| 251 | 50.1 |
| 252 | 18.9 |
| 253 | 395 |
| 254 | 684 |
| 255 | 7.94 |
| 256 | 73.3 |
| 257 | 52.3 |
| 258 | 20.4 |
| 259 | 27.3 |
| 260 | 69.9 |
| 261 | 75.3 |
| 262 | 419 |
| 263 | 23.1 |
| 264 | 3720 |
| 265 | 496 |
| 266 | 231 |
| 267 | 29.3 |
| 268 | 167 |
| 269 | 28.9 |
| 270 | 223 |
| 271 | 23.3 |
| 272 | 155 |
| 273 | 885 |
| 274 | 57.9 |
| 275 | 525 |
| 276 | 71.9 |
| 277 | 101 |
| 278 | 66.4 |
| 279 | 19.3 |
| 280 | 173 |
| 281 | 121 |
| 282 | 266 |
| 283 | 38.6 |
| 284 | 71.6 |
| 285 | 2330 |
| 286 | 88.9 |
| 287 | 139 |
| 288 | 26.7 |
| 289 | 92 |
| 290 | 41.6 |
| 291 | 19 |
| 292 | 152 |
| 293 | 77.7 |
| 294 | 314 |
| 295 | 19.6 |
| 296 | 1660 |
| 297 | 33.2 |
| 298 | 47.2 |
| 299 | 2250 |
| 300 | 415 |
| 301 | 18.9 |
| 302 | 30.9 |
| 303 | 65.9 |
| 304 | 82.6 |
| 305 | >1080 |
| 306 | >10000 |
| 307 | 49.7 |
| 308 | 56.7 |
| 309 | 37.7 |
| 310 | 219 |
| 311 | 513 |
| 312 | 30.7 |
| 314 | 30.7 |
| 315 | 15.4 |
| 317 | >2670 |
| 318 | 924 |
| 319 | 20.1 |
| 320 | 413 |
| 321 | 5.5 |
| 322 | 175 |
| 323 | 566 |
| 324 | 91.1 |
| 325 | 379 |
| 326 | <1.83 |
| 327 | 11.1 |
| 329 | 556 |
| 330 | 875 |
| 331 | 4.2 |
| 332 | 5.4 |
| 333 | 262 |
| 335 | >10000 |
| 336 | 1330 |
| 337 | 55.5 |
| 338 | <1.71 |
| 339 | 261 |
| 340 | 178 |
| 341 | >6080 |
| 342 | 550 |
| 343 | >3590 |
| 344 | >9460 |
| 345 | 462 |
| 346 | 309 |
| 347 | 20.8 |
| 348 | 28.8 |
| 349 | 13.6 |
| 350 | 37 |
| 351 | 13.6 |
| 352 | >10000 |
| 353 | 10 |
| 354 | 70.4 |
| 355 | 9.9 |
| 356 | 98.9 |
| 357 | >2640 |
| 358 | 3.8 |
| 359 | 363 |
| 360 | 625 |
| 361 | 14.5 |
| 362 | 35.6 |
| 363 | 33.2 |
| 364 | 104 |
| 365 | 244 |
| 366 | 2.8 |
| 367 | 18 |

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound of Formula (J): 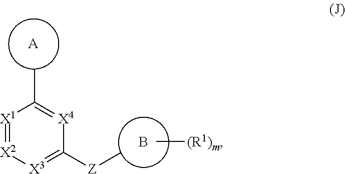
or a pharmaceutically acceptable salt thereof, wherein:
Ring A has the structure: 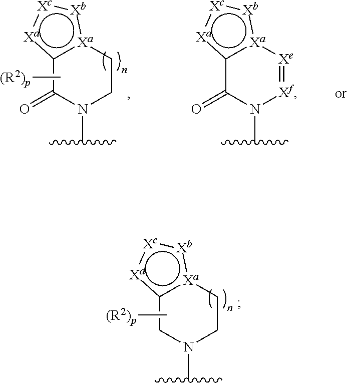
X^{a} is C or N;
X^{b}, X^{c} and X^{d} are each independently CH, C(R²), C(O), N, N(R^{N}), or O;
X^{e} and X^{f} are each independently CH, C(R²), or N;
wherein when X^{a} is C, at least two of X^{b}, X^{c} and X^{d} are independently N, N(R^{N}), or O, and
when X^{a} is N, at least one of X^{b}, X^{c} and X^{d} is N and at least one of X^{b}, X^{c} and X^{d} is C(R²);
each R² is independently H, deuterium, halogen, amino, C1-C6 alkyl, C1-C6 deuteroalkyl, C1-C6 haloalkyl, C1-C6 alkoxy, C1-C6 deuteroalkoxy, C1-C6 hydroxyalkyl, C1-C6 alkylamino, C2-C6 hydroxyalkoxy, C2-C6 alkoxyalkoxy, C2-C6 hydroxyalkylamino, C1-C6 haloalkoxy, (benzyloxy)(C1-C6 alkyl), —C(═O)R^{2b}, —C(═O)OR^{2b}, —C(═O)—NR^{2b}R^{2c}, —NR^{2c}C(═O)R^{2b}, —NR^{2c}—C(═O)OR^{2b}, —NR^{2d}C(═O)—NR^{2b}R^{2c}, —SR^{2b}, —S(═O)R^{2b}, —S(═O)₂R^{2b}, —S(═O)—NR^{2b}R^{2c}, —S(═O)₂—NR^{2b}R^{2c}, —S(═O)(═NH)—NR^{2b}R^{2c}, —NR^{2c}S(═O)R^{2b}, —NR^{2c}S(═O)₂R^{2b}, —NR^{2d}—S(═O)—NR^{2b}R^{2c}, —NR^{2d}S(═O)₂—NR^{2b}R^{2c}, —NR^{2d}S(═O)(═NH)—NR^{2b}R^{2c}, —(C1-C6 alkyl)-C(═O)R^{2b}, —(C1-C6 alkyl)-C(═O)OR^{2b}, —(C1-C6 alkyl)-C(═O)—NR^{2b}R^{2c}, —(C1-C6 alkyl)-S(═O)R^{2b}, —(C1-C6 alkyl)-S(═O)₂R^{2b}, —(C1-C6 alkyl)-S(═O)—NR^{2b}R^{2c}, —(C1-C6 alkyl)-S(═O)₂—NR^{2b}R^{2c}, —(C1-C6 alkyl)-S(═O)(═NH)—NR^{2b}R^{2c}, C3-6 cycloalkyl, heterocyclyl, —(C1-C6 alkyl) (C3-6 cycloalkyl), or —(C1-C6 alkyl) (heterocyclyl), wherein the cycloalkyl, heterocyclyl, (alkyl) cycloalkyl, and (alkyl) heterocyclyl are each independently substituted with 0, 1, 2, or 3 R^{2a},
each R^{2a} is independently deuterium, halogen, hydroxy, C1-C6 alkyl, C1-C6 deuteroalkyl, C1-C6 haloalkyl, C1-C6 alkoxy, C1-C6 deuteroalkoxy, C1-C6 hydroxyalkyl, C1-C6 alkylamino, C2-C6 hydroxyalkoxy, C2-C6 alkoxyalkoxy, C2-C6 hydroxyalkylamino, or C1-C6 haloalkoxy;
R^{2b}, R^{2c}, and R^{2d} are each independently H, C1-C6 alkyl, C1-C6 deuteroalkyl, or C1-C6 hydroxyalkyl;
or, alternatively, R^{2b} and R^{2c}, together with the nitrogen to which they are attached, form a heterocyclyl substituted with 0, 1, 2, or 3 halogen, hydroxy, C1-C6 alkyl, C1-C6 deuteroalkyl, or C1-C6 hydroxyalkyl;
each R^{N} is independently H, C1-C6 alkyl, C1-C6 deuteroalkyl, C1-C6 hydroxyalkyl, (benzyloxy)(C1-C6 alkyl), or a nitrogen protecting group;
subscript n is 1 or 2;
subscript p is 0, 1, 2, or 3;
Ring B is phenyl or heteroaryl;
each R¹ is independently halogen, oxo, cyano, C1-C6 alkyl, C1-C6 haloalkyl, C1-C6 alkoxy, C1-C6 alkylamino, C1-C6 haloalkoxy, C3-C6 cycloalkyl, heterocyclyl, or SF₅, wherein the cyclohexyl and heterocyclyl are each independently substituted with 0, 1, 2, or 3 R^{1a}; or
two adjacent R¹, together with the atoms to which they are attached, form a heterocyclyl or heteroaryl ring substituted with 0, 1, 2, or 3 R^{1a},
each R^{1a} is independently halogen, oxo, hydroxy, C1-C6 alkyl, C1-C6 haloalkyl, or C1-C6 hydroxyalkyl;
subscript m is 1, 2, or 3;
X¹ is N or CR^{X};
X² is N or CR^{X};
X³ is N or CR^{X};
X⁴ is N or CR^{X};
each R^{X} is independently H, halogen, oxo, C1-C6 alkyl, C1-C6 haloalkyl, C1-C6 alkoxy, or C1-C6 haloalkoxy;
Z is —O—, —S—, —S(═O)—, —S(O₂)—, —C(R^{A}R^{B})—, or —N(R^{C})—;
R^{A} and R^{B} are each independently hydrogen, halogen, hydroxyl, or C1-C6 alkyl;
or R^{A} and R^{B}, together with the carbon atom to which they are attached, form ═CH₂, ═O, or a spirocyclopropyl;
or X⁴ and Z, together with the carbon atom to which they are attached, form 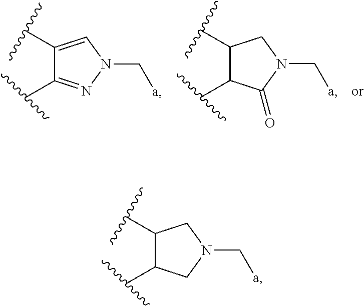
wherein “a” indicates the point 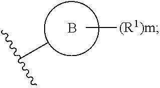
of connection to and R^{C} is hydrogen or C1-C6 alkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

2. The compound of claim 1, or a pharmaceutically acceptable salt thereof, having the structure of Formula (II): 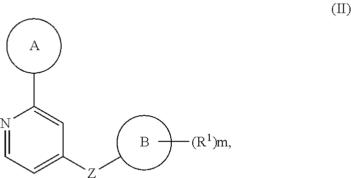
or a pharmaceutically acceptable salt thereof.

3. The compound of claim 1 or 2, or a pharmaceutically acceptable salt thereof, wherein Ring A is:

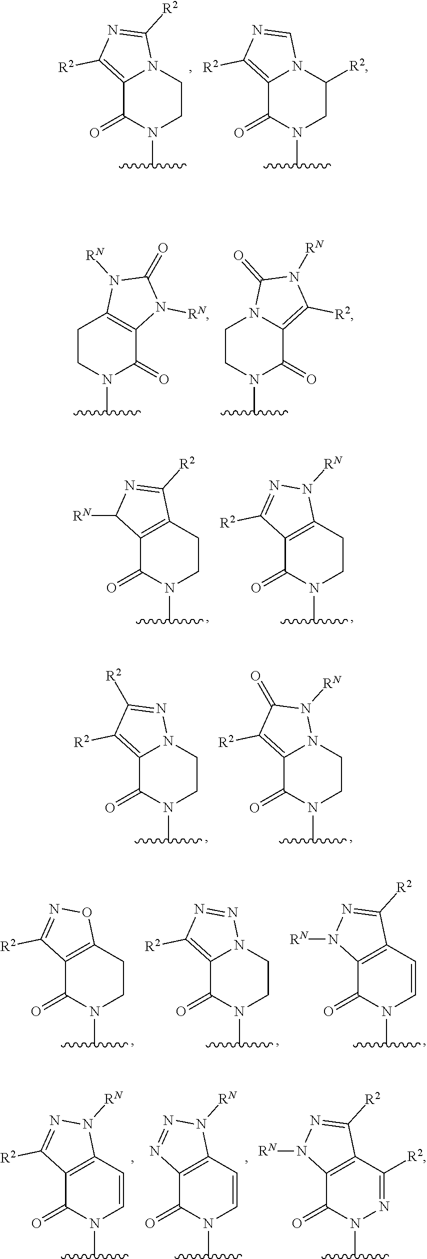

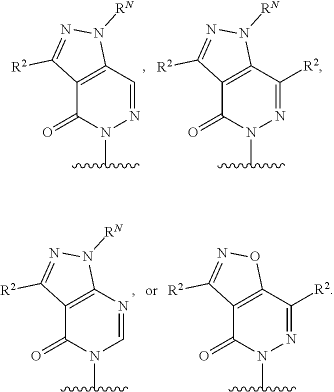

4. The compound of any one of claims 1 to 3, or a pharmaceutically acceptable salt thereof, wherein Ring A is:

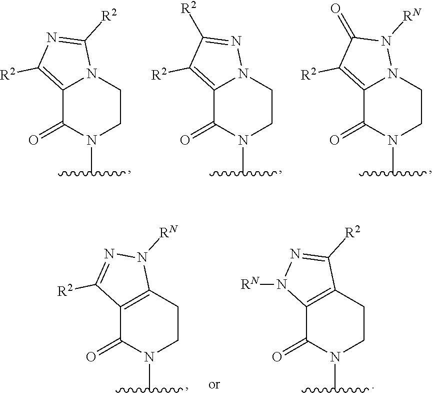

5. The compound of any one of claims 1 to 4, or a pharmaceutically acceptable salt thereof, wherein
each R² is independently C1-C3 alkyl, C1-C3 hydroxyalkyl, C1-C3 alkylamino, C1-C3 hydroxyalkoxy, C1-C3 hydroxyalkylamino, —S(═O)—NR^{2b}R^{2c}, —C(═O)—NR^{2b}R^{2c}, —(C1-C3 alkyl)-C(═O)OR^{2b}, C3-C4 cycloalkyl, or a 4-5 membered heterocyclyl having 1 heteroatom N, wherein the cycloalkyl and heterocyclyl are each independently substituted with 1 R^{2a} substituent;
R^{2a} is hydroxy or C1-C3 hydroxyalkyl;
R^{2b} and R^{2c} are each H, C1-C3 alkyl, or C1-C3 hydroxyalkyl;
or, alternatively, R^{2b} and R^{2c}, together with the nitrogen to which they are attached, form a heterocyclyl substituted with hydroxy or C1-C3 hydroxyalkyl; and
R^{N} is H, C1-C3 alkyl, or C1-C3 hydroxyalkyl.

6. The compound of any one of claims 1 to 5, or a pharmaceutically acceptable salt thereof, having the structure of Formula (IIa): 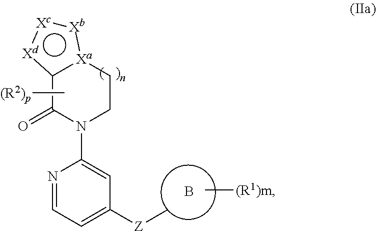
or a pharmaceutically acceptable salt thereof, wherein
X^{a} is C or N;
X^{b} is C(R²) or N(R^{N});
X^{c} and X^{d} are each independently CH, C(R²), C(O), N, or N(R^{N});
wherein when X^{a} is C, at least two of X^{b}, X^{c} and X^{d} are independently N, or N(R^{N}), and
when X^{a} is N, at least one of X^{b}, X^{c} and X^{d} is N and at least one of X^{b}, X^{c} and X^{d} is C(R²);
subscript n is 1 or 2;
Z is —O—, —S—, or —C(R^{A}R^{B})—;
R^{A} and R^{B} are each independently hydrogen, halogen, or hydroxyl;
or R^{A} and R^{B}, together with the carbon atom to which they are attached, form ═O;
Ring B is phenyl or heteroaryl;
each R¹ is independently halogen, cyano, C1-C3 alkyl, C1-C3 haloalkyl, C1-C3 alkoxy, C1-C3 alkylamino, C1-C3 haloalkoxy, C3-C4 cycloalkyl, a 4-5 membered heterocyclyl having 1 heteroatom O, or SF₅, wherein the cyclohexyl and heterocyclyl are each independently substituted with 0, 1, 2 R^{1a}; or
two adjacent R¹, together with the atoms to which they are attached, form a heterocyclyl or heteroaryl ring substituted with 0, 1, or 2 R^{1ª;}
each R^{1a} is independently halogen, C1-C3 alkyl, or C1-C3 haloalkyl;
subscript m is 1, 2, or 3;
each R² is independently C1-C3 alkyl, C1-C3 hydroxyalkyl, C1-C3 hydroxyalkoxy, C1-C3 hydroxyalkylamino, —C(═O)—NR^{2b}R²⁰, —(C1-C6 alkyl)-C(═O)OR^{2b}, C3-C4 cycloalkyl, or a 4-5 membered heterocyclyl having 1 heteroatom N, wherein the cycloalkyl and heterocyclyl are each independently substituted with 1 R^{2a} substituent;
R^{2a} is hydroxy or C1-C3 hydroxyalkyl;
R^{2b} and R^{2c} are each H, C1-C3 alkyl, or C1-C3 hydroxyalkyl;
or, alternatively, R^{2b} and R^{2c}, together with the nitrogen to which they are attached, form a heterocyclyl substituted with hydroxy or C1-C3 hydroxyalkyl; and
R^{N} is H, C1-C3 alkyl or C1-C3 hydroxyalkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

7. The compound of any one of claims 1 to 6, or a pharmaceutically acceptable salt thereof, having the structure of Formula (IIf): 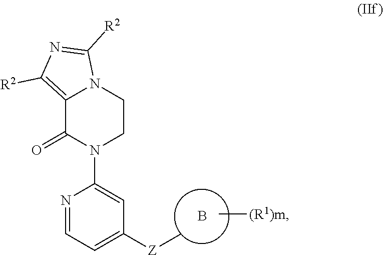
or a pharmaceutically acceptable salt thereof, wherein
Zis —O—, —S—, or —C(R^{A}R^{B})—;
R^{A} and R^{B} are each independently hydrogen, halogen, or hydroxyl;
or R^{A} and R^{B}, together with the carbon atom to which they are attached, form ═O;
Ring B is phenyl or heteroaryl;
each R¹ is independently halogen, cyano, C1-C3 alkyl, C1-C3 haloalkyl, C1-C3 alkoxy, C1-C3 alkylamino, C1-C3 haloalkoxy, C3-C4 cycloalkyl, a 4-5 membered heterocyclyl having 1 heteroatom O, or SF₅, wherein the cyclohexyl and heterocyclyl are each independently substituted with 0, 1, 2 R^{1a}; or
two adjacent R¹, together with the atoms to which they are attached, form a heterocyclyl or heteroaryl ring substituted with 0, 1, or 2 R^{1a}, each R^{1a} is independently halogen, C1-C3 alkyl, or C1-C3 haloalkyl;
subscript m is 1, 2, or 3;
each R² is independently H, C1-C3 alkyl, C1-C3 hydroxyalkyl, C1-C3 hydroxyalkoxy, C1-C3 hydroxyalkylamino, —S(═O)—NR^{2b}R^{2c}, —C(═O)—NR^{2b}R^{2c}, —(C1-C6 alkyl)-C(═O)OR^{2b}, C3-C4 cycloalkyl, or a 4-5 membered heterocyclyl having 1 heteroatom N, wherein the cycloalkyl and heterocyclyl are each independently substituted with 1 R^{2a} substituent;
R^{2a} is hydroxy or C1-C3 hydroxyalkyl; and
R^{2b} and R^{2c} are each H, C1-C3 alkyl, or C1-C3 hydroxyalkyl;
or, alternatively, R^{2b} and R^{2c}, together with the nitrogen to which they are attached, form a heterocyclyl substituted with hydroxy or C1-C3 hydroxyalkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

8. The compound of any one of claims 1 to 6, or a pharmaceutically acceptable salt thereof, having the structure of Formula (IIg): 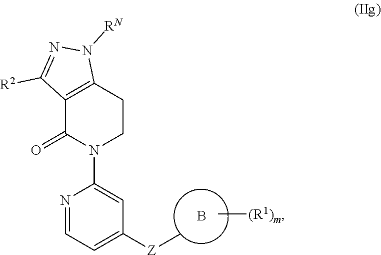
or a pharmaceutically acceptable salt thereof, wherein
R^{A} and R^{B} are each independently hydrogen, halogen, or hydroxyl;
or R^{A} and R^{B}, together with the carbon atom to which they are attached, form ═O;
Ring B is phenyl or heteroaryl;
each R¹ is independently halogen, cyano, C1-C3 alkyl, C1-C3 haloalkyl, C1-C3 alkoxy, C1-C3 alkylamino, C1-C3 haloalkoxy, C3-C4 cycloalkyl, a 4-5 membered heterocyclyl having 1 heteroatom O, or SF₅, wherein the cyclohexyl and heterocyclyl are each independently substituted with 0, 1, 2 R^{1a}, or
two adjacent R¹, together with the atoms to which they are attached, form a heterocyclyl or heteroaryl ring substituted with 0, 1, or 2 R^{1a};
each R^{1a} is independently halogen, C1-C3 alkyl, or C1-C3 haloalkyl;
subscript m is 1, 2, or 3; and
R² is C1-C3 alkyl, C1-C3 hydroxyalkyl, or (benzyloxy)(C1-C6 alkyl);
R^{N} is C1-C3 alkyl, C1-C3 hydroxyalkyl, or (benzyloxy)(C1-C6 alkyl);
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

9. The compound of any one of claims 1 to 3, or a pharmaceutically acceptable salt thereof, having the structure of Formula (IId): 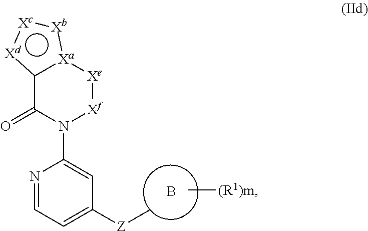
or a pharmaceutically acceptable salt thereof, wherein
X^{a} is C or N;
X^{b} is C(R²) or N(R^{N});
X^{c} and X^{d} are each independently CH, C(R²), C(O), N, or N(R^{N});
Xₑ and X_{f} are each independently CH, C(R²), or N;
wherein when X^{a} is C, at least two of X^{b}, X^{e} and X^{d} are independently N, or N(R^{N}), and when X^{a} is N, at least one of X^{b}, X^{e} and X^{d} is N and at least one of X^{b}, X^{e} and X^{d} is C(R²);
Z is —O—, —S—, or —C(R^{A}R^{B})—;
R^{A} and R^{B} are each independently hydrogen, halogen, or hydroxyl;
or R^{A} and R^{B}, together with the carbon atom to which they are attached, form ═O;
Ring B is phenyl or heteroaryl;
each R¹ is independently halogen, cyano, C1-C3 alkyl, C1-C3 haloalkyl, C1-C3 alkoxy, C1-C3 alkylamino, C1-C3 haloalkoxy, C3-C4 cycloalkyl, a 4-5 membered heterocyclyl having 1 heteroatom O, or SF₅, wherein the cyclohexyl and heterocyclyl are each independently substituted with 0, 1, 2 R^{1a}; or
two adjacent R¹, together with the atoms to which they are attached, form a heterocyclyl or heteroaryl ring substituted with 0, 1, or 2 R^{1a};
each R^{1a} is independently halogen, C1-C3 alkyl, or C1-C3 haloalkyl;
subscript m is 1, 2, or 3;
each R² is amino, C1-C3 hydroxyalkyl, or C1-C3 hydroxyalkylamino; and
R^{N} is C1-C3 alkyl or C1-C3 hydroxyalkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

10. The compound of any one of claims 1 to 6, or a pharmaceutically acceptable salt thereof, having the structure of Formula (IIe): 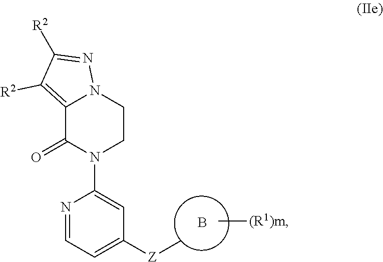
or a pharmaceutically acceptable salt thereof, wherein
Z is —O—, —S—, or —C(R^{A}R^{B})—;
R^{A} and R^{B} are each independently hydrogen, halogen, or hydroxyl;
or R^{A} and R^{B}, together with the carbon atom to which they are attached, form ═O;
Ring B is phenyl or heteroaryl;
each R¹ is independently halogen, cyano, C1-C3 alkyl, C1-C3 haloalkyl, C1-C3 alkoxy, C1-C3 alkylamino, C1-C3 haloalkoxy, C3-C4 cycloalkyl, a 4-5 membered heterocyclyl having 1 heteroatom O, or SF₅, wherein the cyclohexyl and heterocyclyl are each independently substituted with 0, 1, 2 R^{1a}; or
two adjacent R¹, together with the atoms to which they are attached, form a heterocyclyl or heteroaryl ring substituted with 0, 1, or 2 R^{1a};
each R^{1a} is independently halogen, C1-C3 alkyl, or C1-C3 haloalkyl;
subscript m is 1, 2, or 3;
each R² is independently H, C1-C3 alkyl, C1-C3 hydroxyalkyl, C1-C3 hydroxyalkoxy, C1-C3 hydroxyalkylamino, —C(═O)—NR^{2b}R^{2c}, —(C1-C6 alkyl)-C(═O)OR^{2b}, C3-C4 cycloalkyl, or a 4-5 membered heterocyclyl having 1 heteroatom N, wherein the cycloalkyl and heterocyclyl are each independently substituted with 1 R^{2a} substituent; and
R^{2a} is hydroxy or C1-C3 hydroxyalkyl; and
R^{2b} and R^{2c} are each H, C1-C3 alkyl, or C1-C3 hydroxyalkyl;
or, alternatively, R^{2b} and R^{2c}, together with the nitrogen to which they are attached, form a heterocyclyl substituted with hydroxy or C1-C3 hydroxyalkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

11. The compound of any one of claims 1 to 10, or a pharmaceutically acceptable salt thereof, wherein
each R² is independently CH₃, CH₂CH₃, CF₃, CH₂OH, CH₂CH₂OH, CH (OH)CH₂OH, CH₂CH₂CH₂OH, OCH₃, OCH₂CH₃, OCH₂CH₂OH, CH₂NH₂, NH₂, NHCH₃, NHCH₂CH₃, NHCH₂CH₂OH, —S(═O)—NH₂, —(C═O)—NH₂, —(C═O)NHCH₂CH₂OH, —(C═O)N(CH₃)CH₂CH₂OH, —CH₂(C═O)OH,

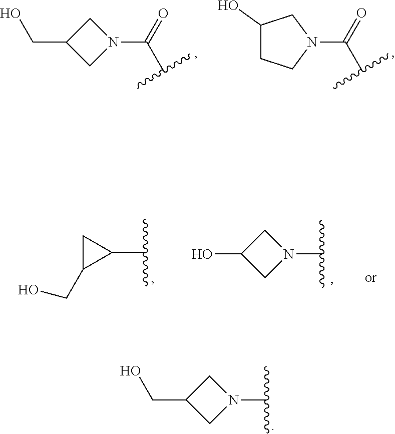 [SMILES: *C(=O)N1CC(CO)C1 ; *C(=O)N1CCC(O)C1 ; *C1CC1CO ; *N1CC(O)C1 ; *N1CC(CO)C1]

12. The compound of any one of claims 1 to 11, or a pharmaceutically acceptable salt thereof, wherein
R² is CH₂OH, CH₂CH₂OH, CH (OH)CH₂OH, CH₂CH₂CH₂OH, OCH₂CH₂OH, NHCH₂CH₂OH, —(C═O)NHCH₂CH₂OH, —(C═O)N(CH₃)CH₂CH₂OH,

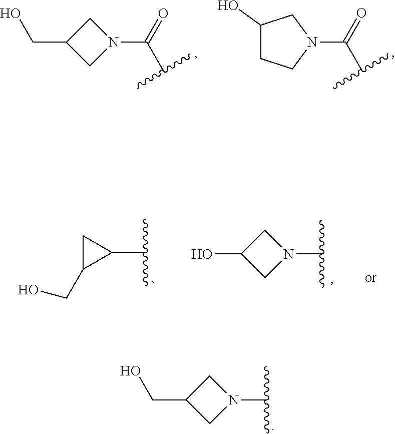 [SMILES: *C(=O)N1CC(CO)C1 ; *C(=O)N1CCC(O)C1 ; *C1CC1CO ; *N1CC(O)C1 ; *N1CC(CO)C1]

13. The compound of any one of claims 1 to 12, or a pharmaceutically acceptable salt thereof, wherein R^{N} is H, CH₃, CH₂CH₃, or CH₂CH₂OH.

14. The compound of claim 1, or a pharmaceutically acceptable salt thereof, wherein R^{N} is CH₂CH₂OH.

15. The compound of any one of claims 1 to 14, or a pharmaceutically acceptable salt thereof, wherein Ring B is pyrazolyl, thiazolyl, phenyl, or pyridyl.

16. The compound of any one of claims 1 to 15, or a pharmaceutically acceptable salt thereof, wherein each R¹ is independently F, Cl, CN, OCH₃, CHF₂, CF₃, NHCH₃, NHCH₂CH₃, OCF₃, cyclopropyl, or

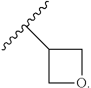 [SMILES: *C1COC1]

17. The compound of claim 16, or a pharmaceutically acceptable salt thereof, wherein each R^{1a} is independently F, OH, CH₃, or CF₃.

18. The compound of any one of claims 1 to 17, or a pharmaceutically acceptable salt thereof, wherein
Z is —O— or —CH₂—;
Ring B is phenyl;
each R¹ is independently F, Cl, cyano, CH₃, CF₂H, CF₃, OCH₃, NHCH₃, NHCH₂CH₃, OCF₃, cyclopropyl, oxetanyl, or SF₅, wherein the cyclopropyl is substituted with 0, 1, 2 F or CH₃; or
two adjacent R¹, together with the atoms to which they are attached, form a tetrahydrofuranyl, tetrahydropyranyl, morpholinyl, pyrazolyl, isoxazolyl, or pyridyl ring substituted with 0, 1 or 2 R^{1a};
each R^{1a} is independently F, CH₃, or CF₃;
subscript m is 1, 2, or 3;
each R² is independently CH₃, CH₂CH₃, CF₃, CH₂OH, CH₂CH₂OH, CH (OH)CH₂OH, CH₂CH₂CH₂OH, OCH₃, OCH₂CH₃, OCH₂CH₂OH, CH₂NH₂, NH₂, NHCH₃, NHCH₂CH₃, NHCH₂CH₂OH, —(C═O)—NH₂, —(C═O)NHCH₂CH₂OH, —(C═O)N(CH₃)CH₂CH₂OH, —CH₂ (C═O)OH,

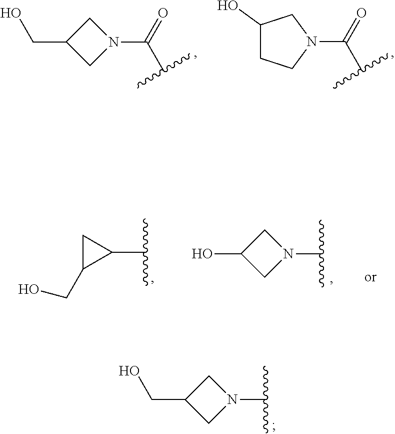 [SMILES: *C(=O)N1CC(CO)C1 ; *C(=O)N1CCC(O)C1 ; *C1CC1CO ; *N1CC(O)C1 ; *N1CC(CO)C1]

19. The compound of any one of claims 1 to 17, or a pharmaceutically acceptable salt thereof, wherein the moiety 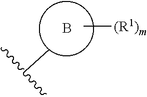
is

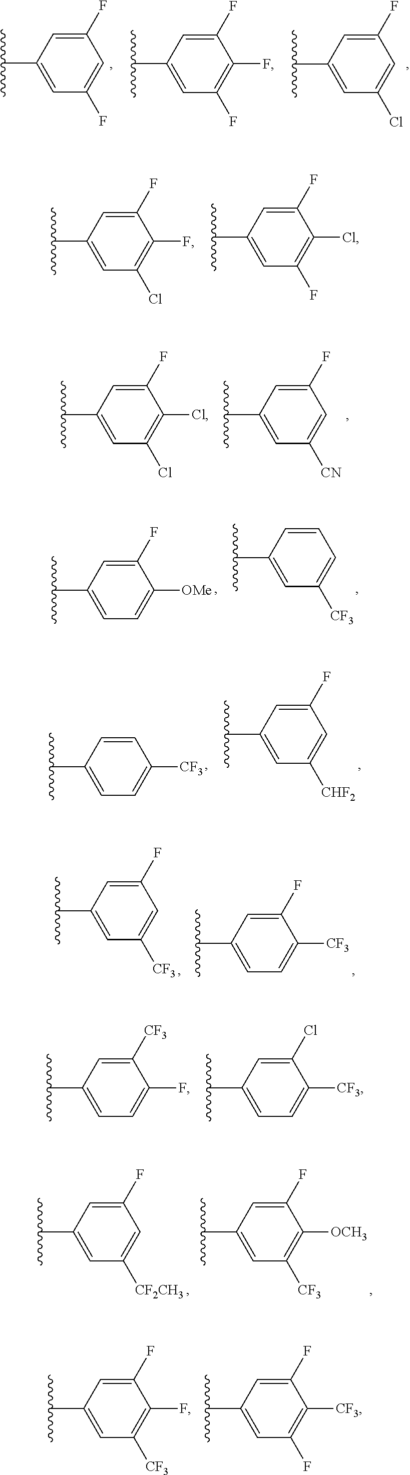

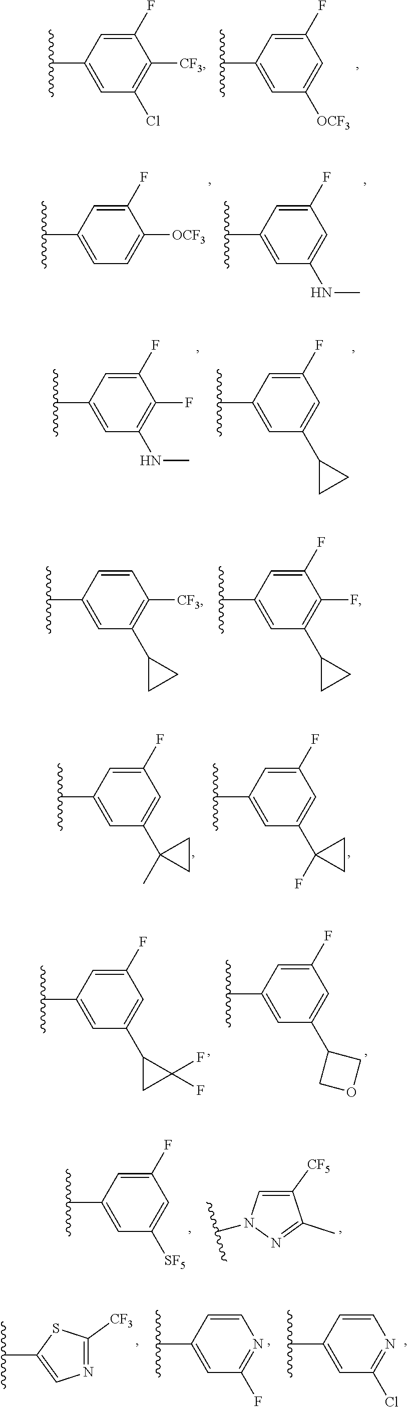

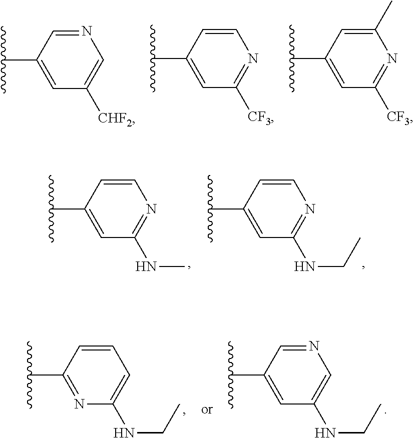

20. The compound of any one of claims 1 to 17, or a pharmaceutically acceptable salt thereof, wherein the moiety 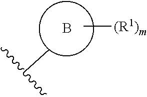
is

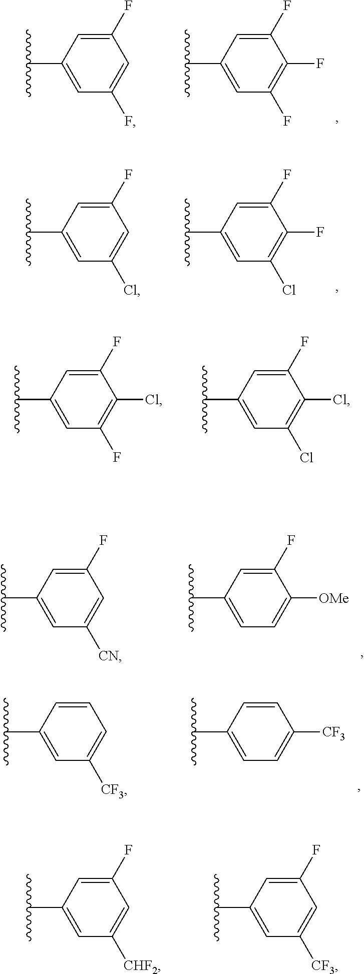

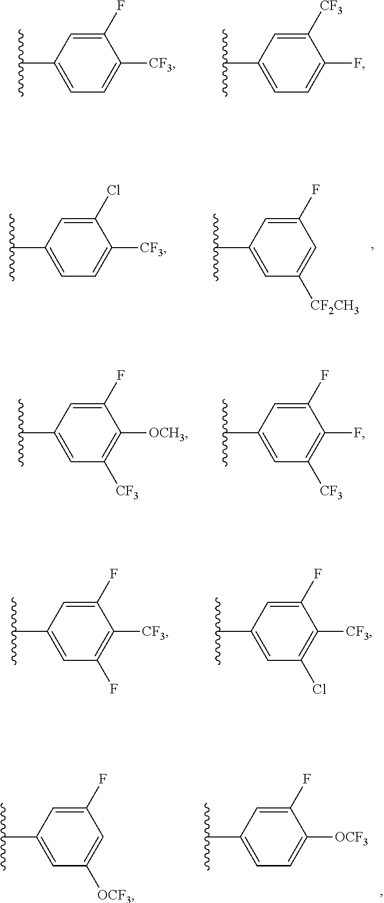

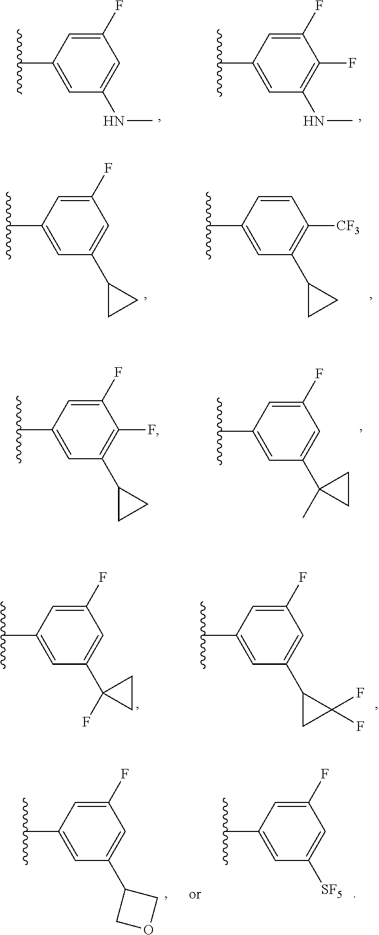

21. The compound of any one of claims 1 to 17, or a pharmaceutically acceptable salt thereof, wherein the moiety 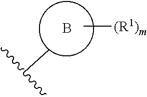
is

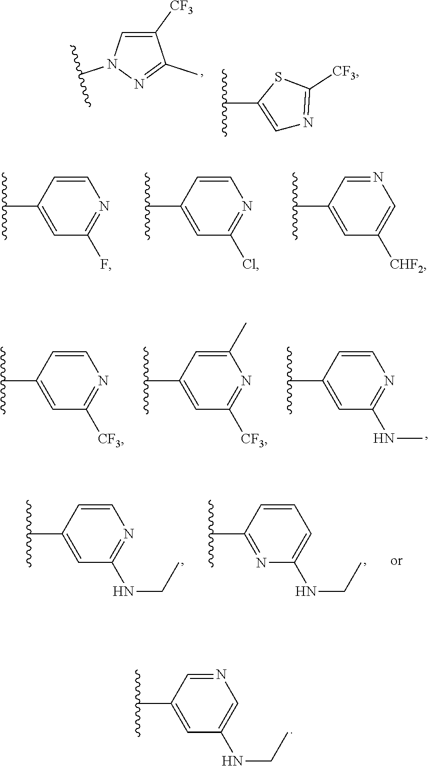

22. The compound of any one of claims 1 to 14, or a pharmaceutically acceptable salt thereof, wherein the moiety 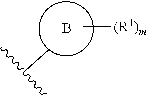
is 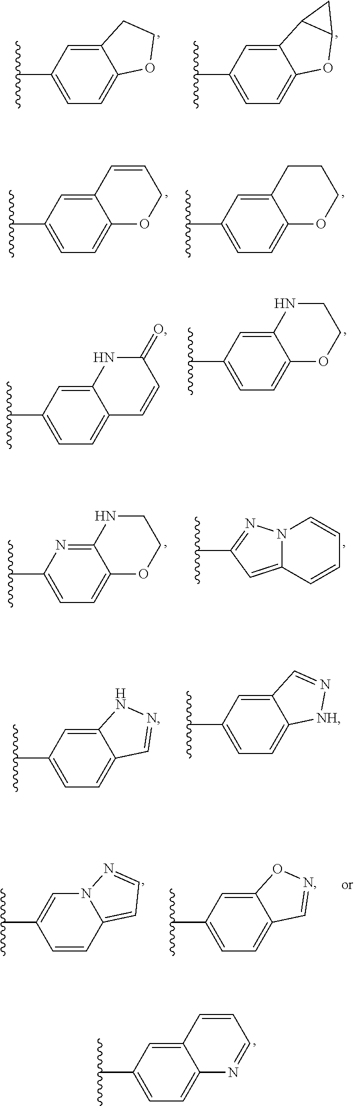
which are each substituted with 0-3 R^{1a}.

23. The compound of claim 22, or a pharmaceutically acceptable salt thereof, wherein R^{1a} is F, OH, CH₃, or CF₃.

24. The compound of claim 22, or a pharmaceutically acceptable salt thereof, wherein the moiety 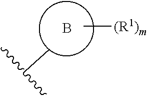
is

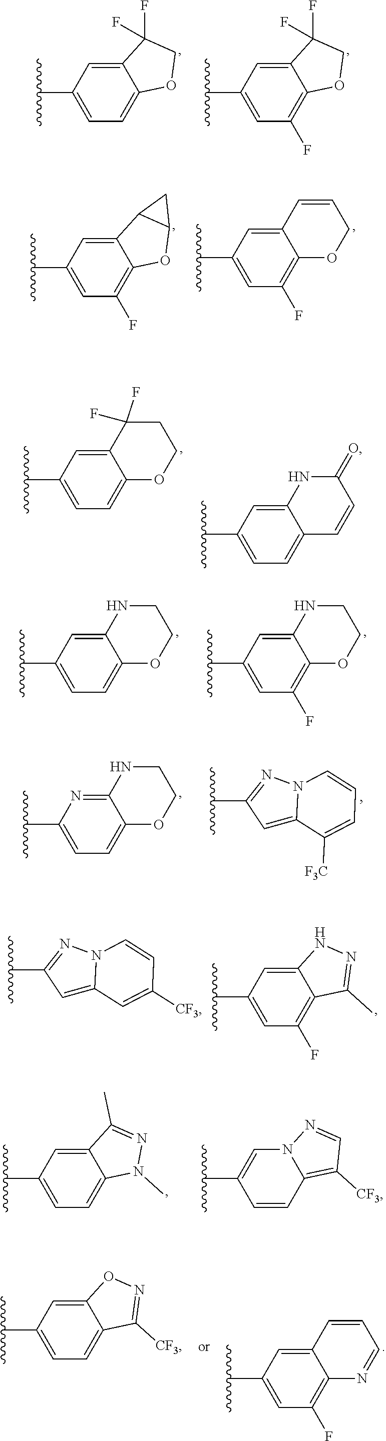

25. The compound of any one of claims 1 to 24, or a pharmaceutically acceptable salt thereof, wherein Z is —O—, —S—, —CH₂—, or —C(═O)—.

26. The compound of any one of claims 1 to 18, or a pharmaceutically acceptable salt thereof, having the structure of Formula (IIb): 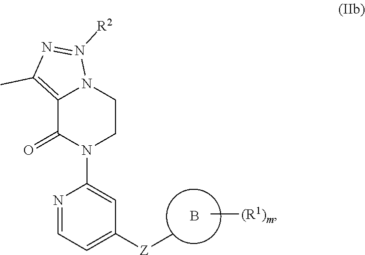
wherein
Z is —O— or —CH₂—;
R² is H, C1-C6 alkyl, C1-C6 haloalkyl, C1-C6 hydroxyalkyl, C1-C6 alkylamino, C2-C6 hydroxyalkoxy, C2-C6 hydroxyalkylamino, —S(═O)—NR^{2b}R^{2c}, —C(═O)—NR^{2b}R^{2c}, —(C1-C6 alkyl)-C(═O)OR^{2b}, C3-6 cycloalkyl, or heterocyclyl, wherein the cycloalkyl and heterocyclyl are each independently substituted with 0 or 1 R^{2a},
R^{2a} is C1-C6 hydroxyalkyl;
R^{2b} and R^{2c} are each H;
Ring B is phenyl or heteroaryl;
each R¹ is independently halogen, cyano, C1-C6 alkyl, C1-C6 haloalkyl, C1-C6 alkoxy, C1-C6 alkylamino, C1-C6 haloalkoxy, C3-C6 cycloalkyl, heterocyclyl, or SF₅, wherein the cyclohexyl and heterocyclyl are each independently substituted with 0, 1, or 2 R^{1a}; or
two adjacent R¹, together with the atoms to which they are attached, form a heterocyclyl or heteroaryl ring substituted with 0, 1, or 2 R^{1a};
each R^{1a} is independently halogen, hydroxy, C1-C6 alkyl, or C1-C6 haloalkyl; and
subscript m is 1, 2, or 3;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

27. The compound of claim 26, or a pharmaceutically acceptable salt thereof, wherein
R² is H, C1-C3 alkyl, C1-C3 haloalkyl, C1-C3 hydroxyalkyl, C1-C3 alkylamino, C2-C3 hydroxyalkoxy, C2-C3 hydroxyalkylamino, —S(═O)—NR^{2b}R^{2c}, —C(═O)—NR^{2b}R^{2c},
—(C1-C3 alkyl)-C(═O)OR^{2b}, C3-4 cycloalkyl, or 4- to 5-membered heterocyclyl, wherein the cycloalkyl and heterocyclyl are each independently substituted with 0 or 1 R^{2a}.

28. The compound of claim 26 or 27, or a pharmaceutically acceptable salt thereof, wherein
R² is H, CH₃, —CH₂OH, —CH₂CH₂OH, —CH₂CH₂CH₂OH, —CH (OH)CH₂OH, —OCH₂CH₂OH, —NHCH₂CH₂OH, —S(O)NH₂, —C(O)NH₂, —CH₂C(O)OH,

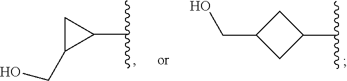 [SMILES: *C1CC1CO ; *C1CC(CO)C1]

29. The compound of any one of claims 26 to 28, or a pharmaceutically acceptable salt thereof, wherein
Ring B is 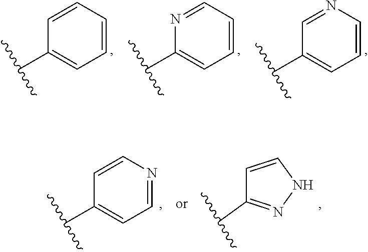
which are each substituted by (R¹)ₘ.

30. The compound of any one of claims 26 to 29, or a pharmaceutically acceptable salt thereof, wherein
each R¹ is independently halogen, cyano, C1-C3 alkyl, C1-C3 haloalkyl, C1-C3 alkoxy, C1-C3 alkylamino, C1-C3 haloalkoxy, C3-C4 cycloalkyl, 4- to 5-membered heterocyclyl, or SF₅, wherein the cyclohexyl and heterocyclyl are each independently substituted with 0, 1, or 2 R^{1a}.

31. The compound of any one of claims 26 to 30, or a pharmaceutically acceptable salt thereof, wherein
each R¹ is independently F, Cl, cyano, CH₃, CF₂H, CF₃, CF₂CH₃, OCH₃, NHCH₃, NHCH₂CH₃, OCF₃, cyclopropyl, oxetanyl, or SF₅, wherein the cyclopropyl is substituted with 0, 1, 2 F or CH₃.

32. The compound of any one of claims 26 to 31, or a pharmaceutically acceptable salt thereof, wherein
two adjacent R¹, together with the atoms to which they are attached, form a tetrahydrofuranyl, tetrahydropyranyl, 2-oxabicyclo[3.1.0]hexyl, morpholinyl, pyrazolyl, isoxazolyl, or pyridyl ring substituted with 0, 1 or 2 R^{1a}, and
each R^{1a} is independently F, hydroxy, CH₃, or CF₃.

33. The compound of any one of claims 26 to 32, or a pharmaceutically acceptable salt thereof, wherein
Z is —O— or —CH₂—;
R² is H, CH₃, —CH₂OH, —CH₂CH₂OH, —CH₂CH₂CH₂OH, —CH (OH)CH₂OH, —OCH₂CH₂OH, —NHCH₂CH₂OH, —S(O)NH₂, —C(O)NH₂, —CH₂C(O)OH, 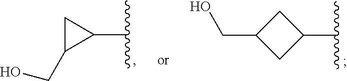 [SMILES: *C1CC1CO ; *C1CC(CO)C1]
Ring B is 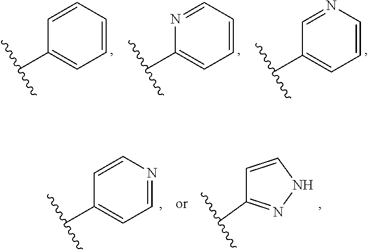
which are each substituted by (R¹)ₘ;
each R¹ is independently F, Cl, cyano, CH₃, CF₂H, CF₃, CF₂CH₃, OCH₃, NHCH₃, NHCH₂CH₃, OCF₃, cyclopropyl, oxetanyl, or SF₅, wherein the cyclopropyl is substituted with 0, 1, 2 F or CH₃; or
two adjacent R¹, together with the atoms to which they are attached, form a tetrahydrofuranyl, tetrahydropyranyl, 2-oxabicyclo[3.1.0]hexyl, morpholinyl, pyrazolyl, isoxazolyl, or pyridyl ring substituted with 0, 1 or 2 R^{1a},
each R^{1a} is independently F, hydroxy, CH₃, or CF₃; and
subscript m is 1, 2, or 3.

34. The compound of any one of claims 26 to 33, or a pharmaceutically acceptable salt thereof, wherein
Z is —O— or —CH₂—;
R² is —CH₂OH, —CH₂CH₂OH, —CH₂CH₂CH₂OH, —CH (OH)CH₂OH, —OCH₂CH₂OH, —NHCH₂CH₂OH, 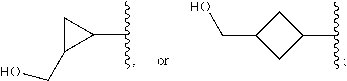 [SMILES: *C1CC1CO ; *C1CC(CO)C1]
Ring B is 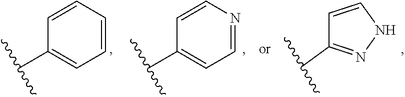
which are each substituted by (R¹)ₘ;
each R¹ is independently F, Cl, cyano, CH₃, CF₂H, CF₃, CF₂CH₃, OCH₃, NHCH₃, NHCH₂CH₃, OCF₃, cyclopropyl, oxetanyl, or SF₅, wherein the cyclopropyl is substituted with 0, 1, 2 F or CH₃; or
two adjacent R¹, together with the atoms to which they are attached, form a tetrahydrofuranyl, tetrahydropyranyl, 2-oxabicyclo[3.1.0]hexyl, morpholinyl, pyrazolyl, isoxazolyl, or pyridyl ring substituted with 0, 1 or 2 R^{1a},
each R^{1a} is independently F, hydroxy, CH₃, or CF₃; and
subscript m is 1, 2, or 3.

35. The compound of any one of claims 26 to 34, or a pharmaceutically acceptable salt thereof, wherein the moiety 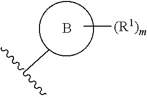
is

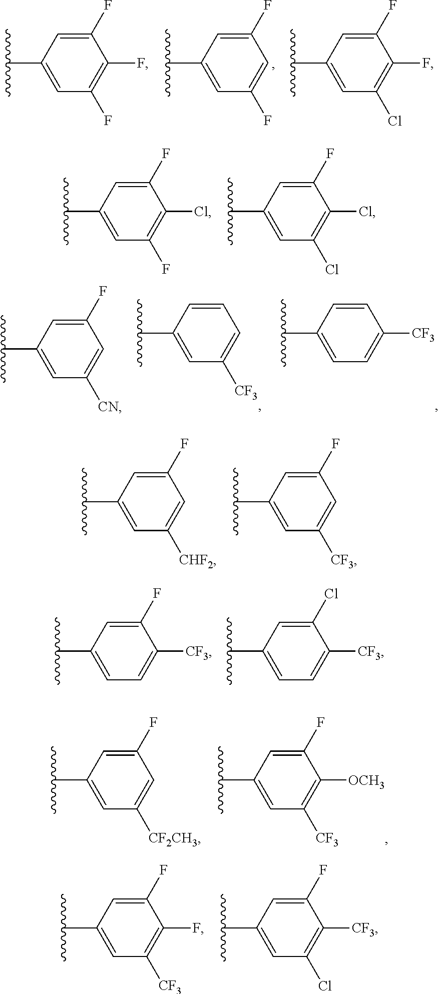

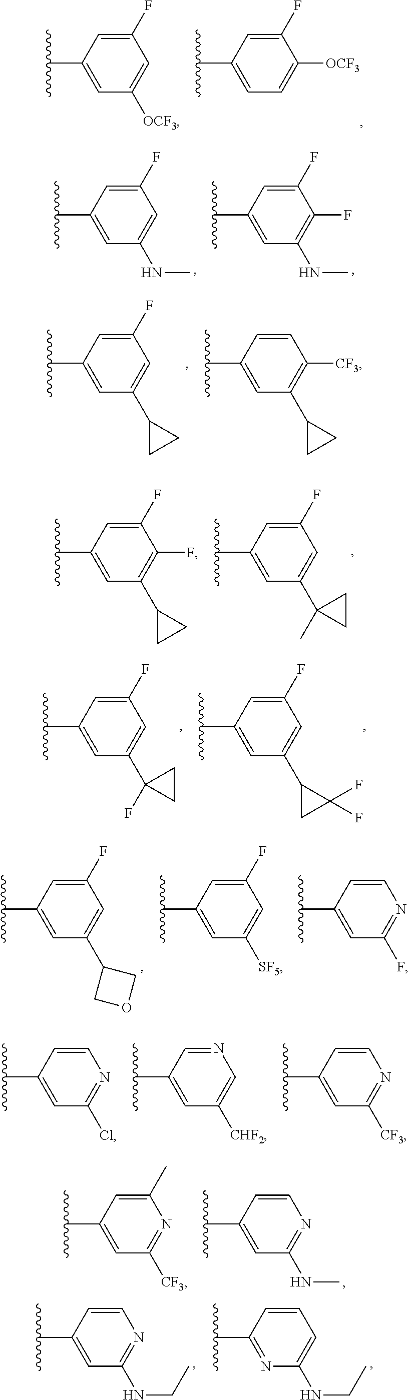

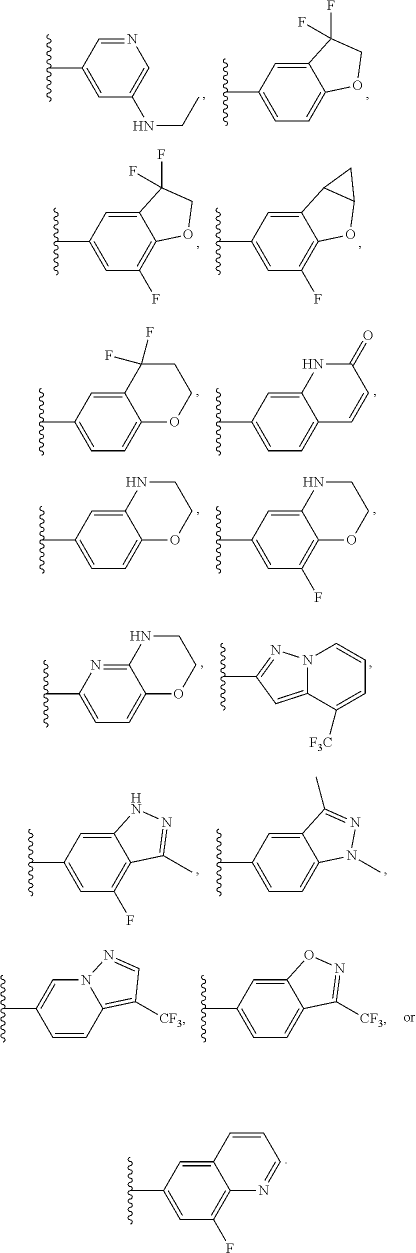

36. The compound of any one of claims 26 to 34, or a pharmaceutically acceptable salt thereof, wherein the moiety 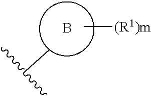
is

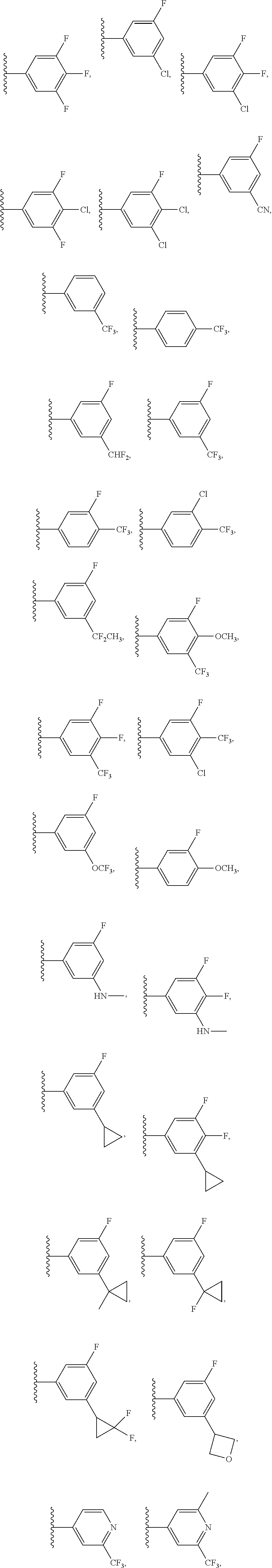

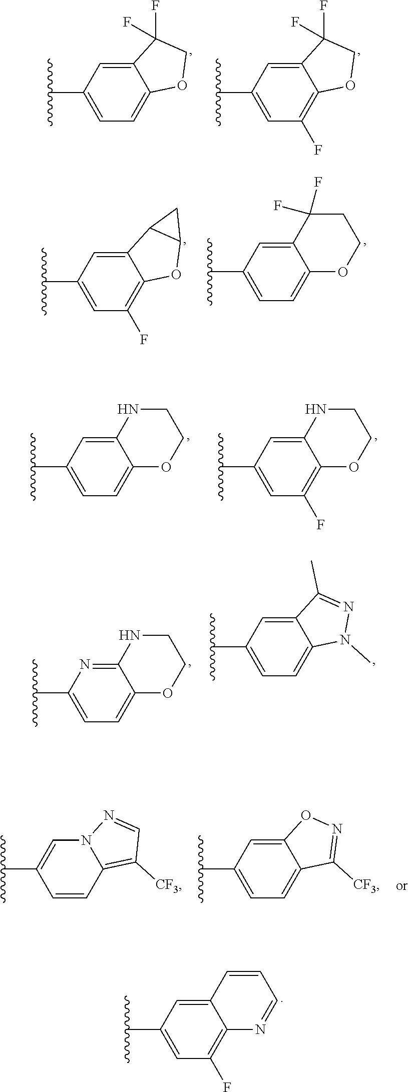

37. The compound of any one of claims 1 to 6, or a pharmaceutically acceptable salt thereof, having the structure of Formula (IIe): 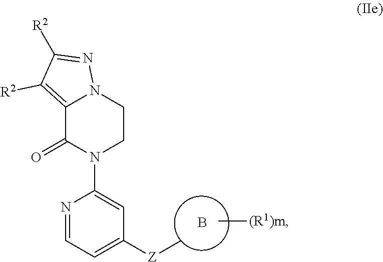
or a pharmaceutically acceptable salt thereof, wherein
Z is —O— or —CH₂—;
Ring B is phenyl;
each R¹ is independently halogen, C1-C6 haloalkyl, or C1-C6 haloalkoxy; or
two adjacent R¹, together with the atoms to which they are attached, form a heterocyclyl ring substituted with 0, 1, or 2 R^{1a},
each R^{1a} is independently halogen;
subscript m is 1, 2, or 3;
each R² is independently H, C1-C6 alkyl, C1-C6 hydroxyalkyl, C1-C6 alkylamino, C1-C6 hydroxyalkoxy, —C(═O)—NR^{2b}R^{2c};
R^{2b} and R^{2c} are each H, C1-C6 alkyl, or C1-C6 hydroxyalkyl;
or, alternatively, R^{2b} and R^{2c}, together with the nitrogen to which they are attached, form a heterocyclyl substituted with hydroxy or C1-C6 hydroxyalkyl;
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

38. The compound of claim 37, or a pharmaceutically acceptable salt thereof, wherein
each R² is independently H, C1-C3 alkyl, C1-C3 hydroxyalkyl, C1-C3 alkylamino, C1-C3 hydroxyalkoxy, —C(═O)—NR^{2b}R^{2c}:
R^{2b} and R^{2c} are each H, C1-C3 alkyl, or C1-C3 hydroxyalkyl;
or, alternatively, R^{2b} and R^{2c}, together with the nitrogen to which they are attached, form a 4- to 5-membered heterocyclyl substituted with hydroxy or C1-C3 hydroxyalkyl.

39. The compound of claim 37 or 38, or a pharmaceutically acceptable salt thereof, wherein
each R² is independently H, CH₃, CH₂CH₃, —CH₂OH, —CH₂CH₂OH, —OCH₂CH₂OH, —CH₂NH₂, —NHCH₃, —NHCH₂CH₃, —C(O)NH₂, —(C═O)NHCH₂CH₂OH, —(C═O)N(CH₃)CH₂CH₂OH,

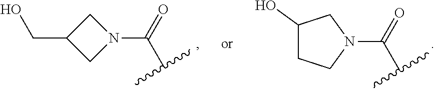 [SMILES: *C(=O)N1CC(CO)C1 ; *C(=O)N1CCC(O)C1]

40. The compound of any one of claims 37 to 39, or a pharmaceutically acceptable salt thereof, wherein
each R¹ is independently halogen, C1-C3 haloalkyl, or C1-C3 haloalkoxy.

41. The compound of any one of claims 37 to 40, or a pharmaceutically acceptable salt thereof, wherein
each R¹ is independently F, Cl, CF₃, or OCF₃; or
two adjacent R¹, together with the atoms to which they are attached, form a tetrahydrofuranyl or morpholinyl ring substituted with 0, 1 or 2 R^{1a}, and
R^{1a} is F.

42. The compound of any one of claims 37 to 41, or a pharmaceutically acceptable salt thereof, wherein
Z is —O— or —CH₂—;
each R² is independently H, CH₃, CH₂CH₃, —CH₂OH, —CH₂CH₂OH, —OCH₂CH₂OH, —CH₂NH₂, —NHCH₃, —NHCH₂CH₃, —C(O)NH₂, —(C═O)NHCH₂CH₂OH, —(C═O)N(CH₃)CH₂CH₂OH, 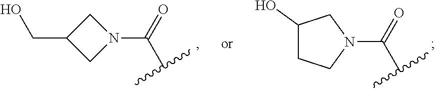 [SMILES: *C(=O)N1CC(CO)C1 ; *C(=O)N1CCC(O)C1]
Ring B is 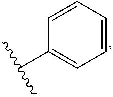 [SMILES: *c1ccccc1]
which is substituted by (R¹)ₘ;
each R¹ is independently F, Cl, CF₃, or OCF₃; or
two adjacent R¹, together with the atoms to which they are attached, form a tetrahydrofuranyl or morpholinyl ring substituted with 0, 1 or 2 R^{1a},
R^{1a} is F; and
subscript m is 1, 2, or 3.

43. The compound of any one of claims 37 to 42, or a pharmaceutically acceptable salt thereof, wherein the moiety 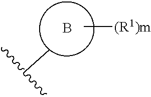
is

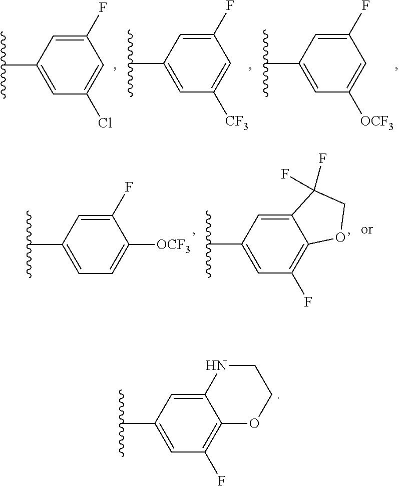

44. The compound of any one of claims 1 to 6, or a pharmaceutically acceptable salt thereof, having the structure of Formula (IIg): 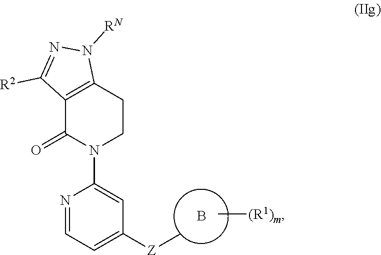
or a pharmaceutically acceptable salt thereof, wherein
Z is —O— or —CH₂—;
Ring B is phenyl or heteroaryl;
each R¹ is independently halogen, cyano, C1-C6 haloalkyl, C1-C6 haloalkoxy, C3-C6 cycloalkyl, or heterocyclyl, wherein the cyclohexyl and heterocyclyl are each independently substituted with 0, 1, 2 R^{1a}; or
two adjacent R¹, together with the atoms to which they are attached, form a heterocyclyl or heteroaryl ring substituted with 0, 1, or 2 R^{1a};
each R^{1a} is independently halogen, C1-C6 alkyl, or C1-C6 haloalkyl;
subscript m is 1, 2, or 3;
R² is C1-C6 alkyl, C1-C6 hydroxyalkyl, or (benzyloxy)(C1-C6 alkyl);
R^{N} is H, C1-C6 alkyl, C1-C6 hydroxyalkyl, or (benzyloxy)(C1-C6 alkyl);
wherein the heterocyclyl in each instance is a 3- to 7-membered ring having one to four heteroatoms each independently N, O, or S; and
the heteroaryl in each instance is a 5- to 6-membered ring having one to four heteroatoms each independently N, O, or S.

45. The compound of claim 44, or a pharmaceutically acceptable salt thereof, wherein
Ring B is 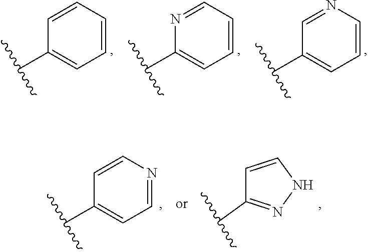
which are each substituted by (R¹)ₘ.

46. The compound of claim 44 or 45, or a pharmaceutically acceptable salt thereof, wherein
each R¹ is independently halogen, cyano, C1-C3 haloalkyl, C1-C3 haloalkoxy, C3-C4 cycloalkyl, or 4- to 5-membered heterocyclyl, wherein the cyclohexyl and heterocyclyl are each independently substituted with 0, 1, 2 R^{1a}.

47. The compound of any one of claims 44 to 46, or a pharmaceutically acceptable salt thereof, wherein
each R^{1a} is independently halogen, C1-C3 alkyl, or C1-C3 haloalkyl.

48. The compound of any one of claims 44 to 47, or a pharmaceutically acceptable salt thereof, wherein
R² is C1-C3 alkyl, C1-C3 hydroxyalkyl, or (benzyloxy)(C1-C3 alkyl).

49. The compound of any one of claims 44 to 48, or a pharmaceutically acceptable salt thereof, wherein
Ring B is 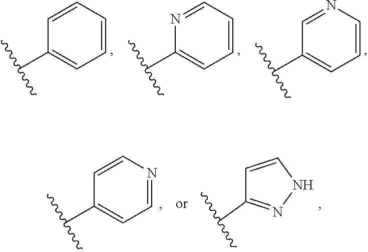
which are each substituted by (R¹)ₘ.

50. The compound of any one of claims 44 to 49, or a pharmaceutically acceptable salt thereof, wherein
each R¹ is independently F, Cl, cyano, CH₃, CF₂H, CF₃, OCF₃, cyclopropyl, oxetanyl, wherein the cyclopropyl is substituted with 0, 1, 2 R^{1a}, or
two adjacent R¹, together with the atoms to which they are attached, form a tetrahydrofuranyl, dihydropyranyl, tetrahydropyranyl, 2-oxabicyclo[3.1.0]hexyl, morpholinyl, pyrazolyl, isoxazolyl, or pyridyl ring substituted with 0, 1 or 2 R^{1a}; and
each R^{1a} is independently F, CH₃, or CF₃.

51. The compound of any one of claims 44 to 50, or a pharmaceutically acceptable salt thereof, wherein
R² is CH₃, CH₂CH₃, —CH₂OH, or —CH₂OBn.

52. The compound of any one of claims 44 to 51, or a pharmaceutically acceptable salt thereof, wherein
R^{N} is H, CH₃, —CH₂CH₂OH, or —CH₂CH₂OBn.

53. The compound of any one of claims 44 to 52, or a pharmaceutically acceptable salt thereof, wherein
Z is —O— or —CH₂—;
Ring B is 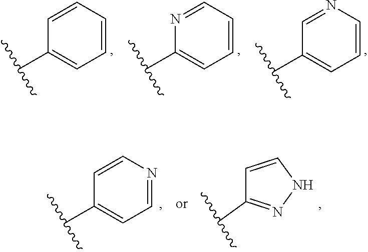
which are each substituted by (R¹)ₘ;
each R¹ is independently F, Cl, cyano, CH₃, CF₂H, CF₃, OCF₃, cyclopropyl, oxetanyl, wherein the cyclopropyl is substituted with 0, 1, 2 R^{1a}; or
two adjacent R¹, together with the atoms to which they are attached, form a tetrahydrofuranyl, dihydropyranyl, tetrahydropyranyl, 2-oxabicyclo[3.1.0]hexyl, morpholinyl, pyrazolyl, isoxazolyl, or pyridyl ring substituted with 0, 1 or 2 R^{1a};
each R^{1a} is independently F, CH₃, or CF₃;
subscript m is 1, 2, or 3;
R² is CH₃, CH₂CH₃, —CH₂OH, or —CH₂OBn; and
R^{N} is H, CH₃, —CH₂CH₂OH, or —CH₂CH₂OBn.

54. The compound of any one of claims 44 to 53, or a pharmaceutically acceptable salt thereof, wherein the moiety 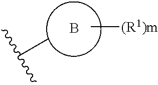
is

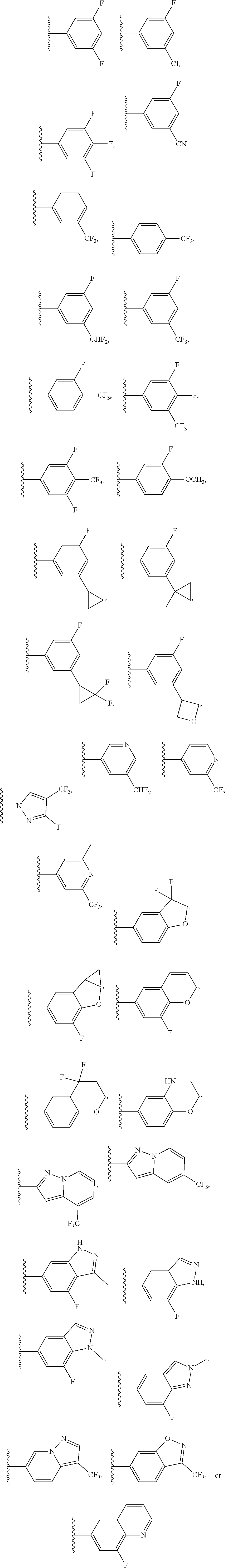

55. The compound of claim 1, or a pharmaceutically acceptable salt thereof, having the structure of Formula (I): 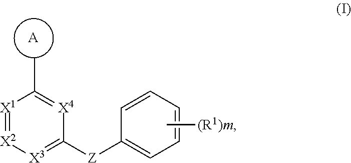
or a pharmaceutically acceptable salt thereof, wherein:
each R¹ is independently selected from halogen, cyano, C1-C6 alkyl, C1-C6 haloalkyl, C1-C6 alkoxy, and C1-C6 haloalkoxy; or
two adjacent R¹, together with the carbon atoms to which they are attached, form a 5-membered heteroaryl ring optionally substituted with C1-C6 alkyl;
m is 1, 2, or 3;
X¹ is N or CR^{X};
X² is N or CR^{X};
X³ is N or CR^{X};
X⁴ is N or CR^{X};
each R^{X} is independently selected from H, halogen, C1-C6 alkyl, C1-C6 haloalkyl, C1-C6 alkoxy, and C1-C6 haloalkoxy;
Z is —O—, —S—, —S(═O)—, —S(O₂)—, —C(R^{A}R^{B})—, or —N(R^{C})—;
R^{A} and R^{B} are independently selected from hydrogen, halogen, hydroxyl, and C1-C6 alkyl;
or R^{A} and R^{B}, together with the carbon atom to which they are attached, form ═CH₂, ═O, or a spirocyclopropyl;
or X⁴ and Z, together with the carbon atom to which they are attached, form 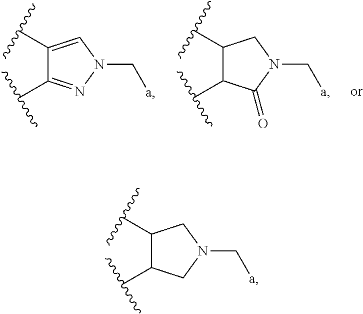
wherein “a” indicates the point of connection to 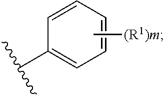
R^{C} is hydrogen or C1-C6 alkyl;
Ring A is selected from: 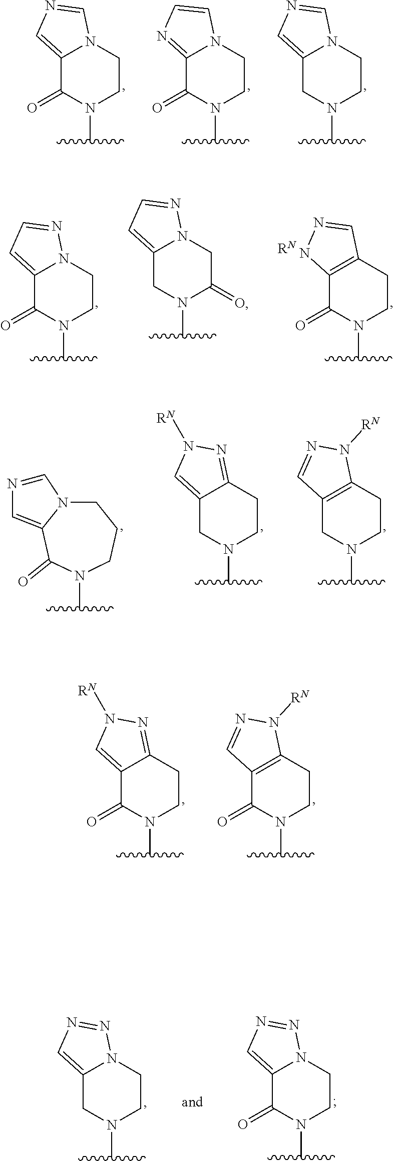
each optionally substituted with 1-3 substituents independently selected from deuterium, halogen, C1-C6 alkyl, C1-C6 deuteroalkyl, C1-C6 haloalkyl, C1-C6 alkoxy, C1-C6 deuteroalkoxy, C1-C6 hydroxyalkyl, and C1-C6 haloalkoxy; and
R^{N} is H, C1-C6 alkyl, C1-C6 deuteroalkyl, or a nitrogen protecting group.

56. The compound of claim 55, or a pharmaceutically acceptable salt thereof, wherein Ring A is 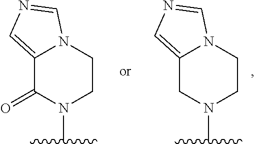 [SMILES: *N1CCn2cncc2C1=O ; *N1CCn2cncc2C1]
each of which is substituted with 0-3 R² substituents independently selected from deuterium, halogen, C1-C6 alkyl, C1-C6 deuteroalkyl, C1-C6 haloalkyl, C1-C6 alkoxy, C1-C6 deuteroalkoxy, and C1-C6 haloalkoxy.

57. The compound of claim 56, or a pharmaceutically acceptable salt thereof, wherein Ring A is 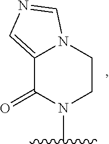 [SMILES: *N1CCn2cncc2C1=O]
which is substituted with 0-3 R² substituents independently selected from deuterium, halogen, C1-C6 alkyl, C1-C6 deuteroalkyl, C1-C6 haloalkyl, C1-C6 alkoxy, C1-C6 deuteroalkoxy, and C1-C6 haloalkoxy.

58. The compound of any one of claims 55-57, or a pharmaceutically acceptable salt thereof, wherein Ring A is

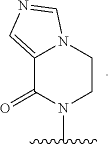 [SMILES: *N1CCn2cncc2C1=O]

59. The compound of any one of claims 55-57, or a pharmaceutically acceptable salt thereof, wherein Ring A is: 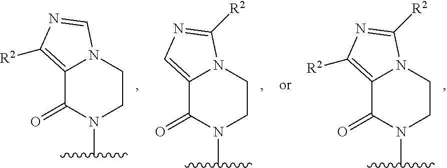
wherein each occurrence of R² is independently deuterium, halogen, C1-C6 alkyl, C1-C6 deuteroalkyl, C1-C6 haloalkyl, C1-C6 alkoxy, C1-C6 deuteroalkoxy, or C1-C6 haloalkoxy.

60. The compound of any one of claims 57 and 59, or a pharmaceutically acceptable salt thereof, wherein Ring A is: 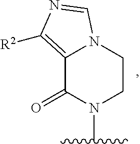
wherein R² is deuterium, halogen, C1-C6 alkyl, C1-C6 deuteroalkyl, C1-C6 haloalkyl, C1-C6 alkoxy, C1-C6 deuteroalkoxy, or C1-C6 haloalkoxy.

61. The compound of claim 60, or a pharmaceutically acceptable salt thereof, wherein R² is C1-C6 alkyl.

62. The compound of claim 60 or 61, or a pharmaceutically acceptable salt thereof, wherein R² is CH₃.

63. The compound of claim 60, or a pharmaceutically acceptable salt thereof, wherein R² is C1-C6 haloalkyl.

64. The compound of claim 60 or 63, or a pharmaceutically acceptable salt thereof, wherein R² is CHF₂ or CF₃.

65. The compound of claim 60, or a pharmaceutically acceptable salt thereof, wherein R² is C1-C6 alkoxy.

66. The compound of claim 60 or 65, or a pharmaceutically acceptable salt thereof, wherein R² is OCH₃ or OCH₂CH₃.

67. The compound of claim 55, or a pharmaceutically acceptable salt thereof, wherein Ring A is 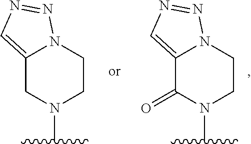 [SMILES: *N1CCn2nncc2C1 ; *N1CCn2nncc2C1=O]
each of which is substituted with 0-3 R² substituents independently deuterium, halogen, C1-C6 alkyl, C1-C6 deuteroalkyl, C1-C6 haloalkyl, C1-C6 alkoxy, C1-C6 deuteroalkoxy, or C1-C6 haloalkoxy.

68. The compound of claim 55, or a pharmaceutically acceptable salt thereof, wherein Ring A is 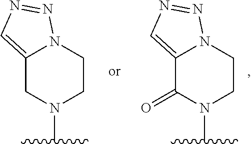 [SMILES: *N1CCn2nncc2C1 ; *N1CCn2nncc2C1=O]
which is substituted with 0-3 R² substituents independently deuterium, halogen, C1-C6 alkyl, C1-C6 deuteroalkyl, C1-C6 haloalkyl, C1-C6 alkoxy, C1-C6 deuteroalkoxy, or C1-C6 haloalkoxy.

69. The compound of claim 55, or a pharmaceutically acceptable salt thereof, wherein Ring A is: 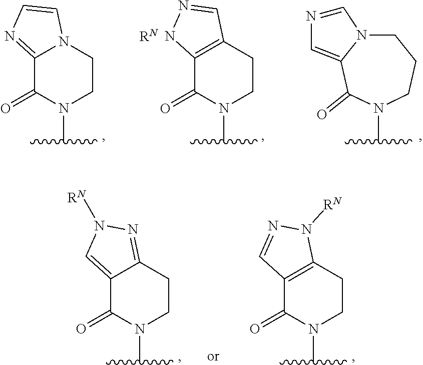
each of which is substituted with 0-3 R² substituents independently deuterium, halogen, C1-C6 alkyl, C1-C6 deuteroalkyl, C1-C6 haloalkyl, C1-C6 alkoxy, C1-C6 deuteroalkoxy, or C1-C6 haloalkoxy.

70. The compound of any one of claims 55-69, or a pharmaceutically acceptable salt thereof, wherein 1-2 of X¹, X², X³, and X⁴ are N.

71. The compound of any one of 55-70, or a pharmaceutically acceptable salt thereof, wherein X¹ is N.

72. The compound of any one of claims 55-71, or a pharmaceutically acceptable salt thereof, wherein X³ is N.

73. The compound of any one of claims 55-72, or a pharmaceutically acceptable salt thereof, wherein X⁴ is CR^{X}.

74. The compound of claim 73, or a pharmaceutically acceptable salt thereof, wherein R^{X} is H.

75. The compound of any one of claims 55-70, or a pharmaceutically acceptable salt thereof, wherein X¹ is N, and X₃ and X⁴ are CR^{X}.

76. The compound of any one of claims 55-70, or a pharmaceutically acceptable salt thereof, wherein X³ is N, and X¹ and X⁴ are CR^{X}.

77. The compound of any one of claims 55-70, or a pharmaceutically acceptable salt thereof, wherein X¹ and X³ are N, and X⁴ is CR^{X}.

78. The compound of any one of claims 75-77, or a pharmaceutically acceptable salt thereof, wherein each occurrence R^{X} is H.

79. The compound of any one of claims 55-78, or a pharmaceutically acceptable salt thereof, wherein X² is CR^{X}.

80. The compound of any one of claims 55-78, or a pharmaceutically acceptable salt thereof, wherein R^{X} is H.

81. The compound of claim 80, or a pharmaceutically acceptable salt thereof, wherein R^{X} is C1-C3 haloalkyl, optionally wherein R^{X} is CF₃.

82. The compound of any one of claims 55-78, or a pharmaceutically acceptable salt thereof, wherein R^{X} is C1-C3 alkoxy, optionally wherein R^{X} is OCH₃.

83. The compound of claim 82, or a pharmaceutically acceptable salt thereof, wherein X² is N.

84. The compound of any one of claims 55-69, or a pharmaceutically acceptable salt thereof, wherein X³ and Z, together with the carbon atom to which they are attached, form 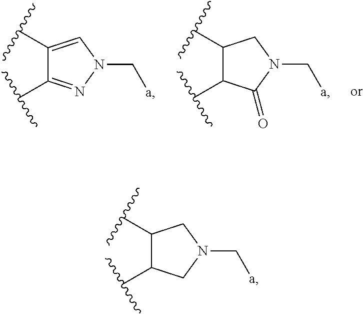
wherein “a” indicates the point of connection to

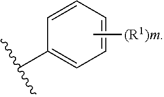

85. The compound of any one of claims 55-84, or a pharmaceutically acceptable salt thereof, wherein Z is —C(R^{A}R^{B})—.

86. The compound of any one of claims 55-85, or a pharmaceutically acceptable salt thereof, wherein Z is —CH₂—.

87. The compound of any one of claims 55-86, or a pharmaceutically acceptable salt thereof, wherein m is 2 or 3.

88. The compound of any one of claims 55-87, or a pharmaceutically acceptable salt thereof, wherein 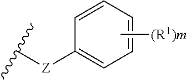
is: 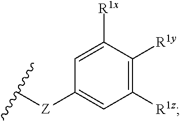
wherein:
each of R^{1x} and R^{1z} is independently selected from halogen, cyano, C1-C6 alkyl, C1-C6 haloalkyl, C1-C6 alkoxy, and C1-C6 haloalkoxy; and
R^{1b} is H or halogen.

89. The compound of claim 88, or a pharmaceutically acceptable salt thereof, wherein R^{1y} is H.

90. The compound of claim 89, or a pharmaceutically acceptable salt thereof, wherein each of R^{1x} and R^{1z} is independently selected from halogen and C1-C6 haloalkyl.

91. The compound of claim 89 or 90, or a pharmaceutically acceptable salt thereof, wherein each of R^{1x} and R^{1z} is independently selected from fluoro, CHF₂, and CF₃.

92. The compound of any one of claims 89-91, or a pharmaceutically acceptable salt thereof, wherein one of R^{1x} and R^{1z} is halogen, and the other of R^{1x} and R^{1z} is C1-C6 haloalkyl.

93. The compound of any one of claims 89-92, or a pharmaceutically acceptable salt thereof, wherein one of R^{1x} and R^{1z} is fluoro, and the other of R^{1x} and R^{1z} is CHF₂ or CF₃.

94. The compound of any one of claims 89-93, or a pharmaceutically acceptable salt thereof, wherein one of R^{1x} and R^{1c} is fluoro, and the other of R^{1x} and R^{1z} is CHF₂.

95. The compound of claim 88, or a pharmaceutically acceptable salt thereof, wherein R^{1y} is halogen.

96. The compound of claim 95, or a pharmaceutically acceptable salt thereof, wherein R^{1y} is fluoro.

97. The compound of claim 95 or 96, or a pharmaceutically acceptable salt thereof, wherein each of R^{1x} and R^{1z} is an independently selected halogen.

98. The compound of any one of claims 95-97, or a pharmaceutically acceptable salt thereof, wherein each of R^{1x} and R^{1z} is independently fluoro or chloro.

99. The compound of any one of claims 95-98, or a pharmaceutically acceptable salt thereof, wherein one of R^{1x} and R^{1z} is independently fluoro, and the other of R^{1x} and R^{1z} is chloro.

100. The compound of claim 55, or a pharmaceutically acceptable salt thereof, wherein:
Ring A is 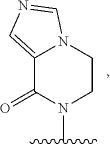 [SMILES: *N1CCn2cncc2C1=O]
which is substituted with 0-3 R² substituents independently selected from deuterium, halogen, C1-C6 alkyl, C1-C6 deuteroalkyl, C1-C6 haloalkyl, C1-C6 alkoxy, C1-C6 deuteroalkoxy, and C1-C6 haloalkoxy;
X¹ is N or CH;
X² is CH;
X³ is N or CH;
X⁴ is CH;
Z is CH₂; and 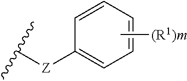
is: 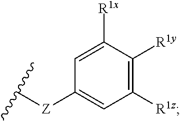
wherein:
each of R^{1x} and R^{1z} is independently selected from halogen, cyano, C1-C6 alkyl, C1-C6 haloalkyl, C1-C6 alkoxy, and C1-C6 haloalkoxy; and
R^{1y} is H or halogen.

101. The compound of any one of claims 1 to 3, or a pharmaceutically acceptable salt thereof, wherein the compound has the structure of a compound in Table 1.

102. The compound of any one of claims 1 to 3, or a pharmaceutically acceptable salt thereof, wherein the compound has the structure:

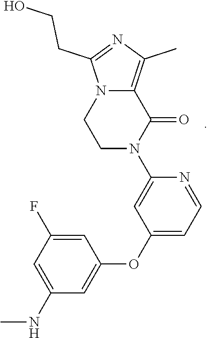 [SMILES: CNc1cc(F)cc(Oc2ccnc(N3CCn4c(CCO)nc(C)c4C3=O)c2)c1]

103. The compound of any one of claims 1 to 3, or a pharmaceutically acceptable salt thereof, wherein the compound has the structure:

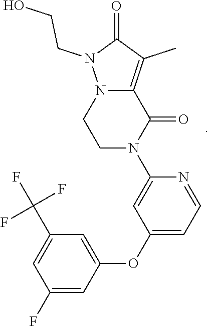 [SMILES: Cc1c2n(n(CCO)c1=O)CCN(c1cc(Oc3cc(F)cc(C(F)(F)F)c3)ccn1)C2=O]

104. The compound of any one of claims 1 to 3, or a pharmaceutically acceptable salt thereof, wherein the compound has the structure:

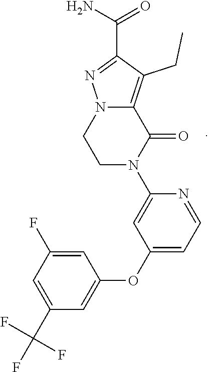 [SMILES: CCc1c(C(N)=O)nn2c1C(=O)N(c1cc(Oc3cc(F)cc(C(F)(F)F)c3)ccn1)CC2]

105. The compound of any one of claims 1 to 3, or a pharmaceutically acceptable salt thereof, wherein the compound has the structure:

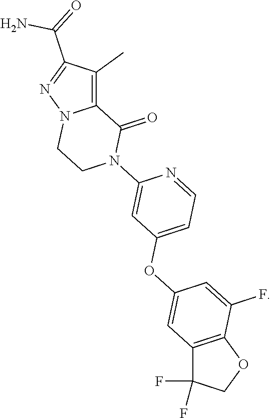 [SMILES: Cc1c(C(N)=O)nn2c1C(=O)N(c1cc(Oc3cc(F)c4c(c3)C(F)(F)CO4)ccn1)CC2]

106. The compound of any one of claims 1 to 3, or a pharmaceutically acceptable salt thereof, wherein the compound has the structure:

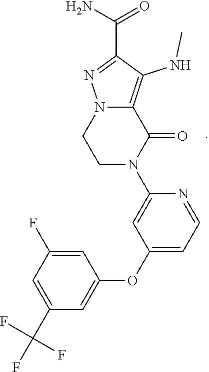 [SMILES: CNc1c(C(N)=O)nn2c1C(=O)N(c1cc(Oc3cc(F)cc(C(F)(F)F)c3)ccn1)CC2]

107. The compound of any one of claims 1 to 3, or a pharmaceutically acceptable salt thereof, wherein the compound has the structure:

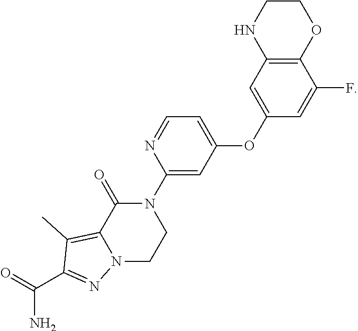 [SMILES: Cc1c(C(N)=O)nn2c1C(=O)N(c1cc(Oc3cc(F)c4c(c3)NCCO4)ccn1)CC2]

108. The compound of any one of claims 1 to 3, or a pharmaceutically acceptable salt thereof, wherein the compound has the structure:

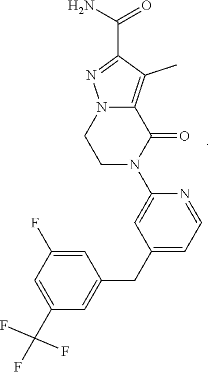 [SMILES: Cc1c(C(N)=O)nn2c1C(=O)N(c1cc(Cc3cc(F)cc(C(F)(F)F)c3)ccn1)CC2]

109. The compound of any one of claims 1 to 3, or a pharmaceutically acceptable salt thereof, wherein the compound has the structure:

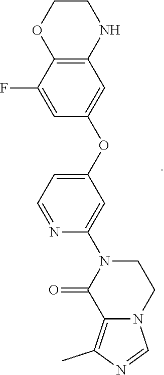 [SMILES: Cc1ncn2c1C(=O)N(c1cc(Oc3cc(F)c4c(c3)NCCO4)ccn1)CC2]

110. The compound of any one of claims 1 to 3, or a pharmaceutically acceptable salt thereof, wherein the compound has the structure:

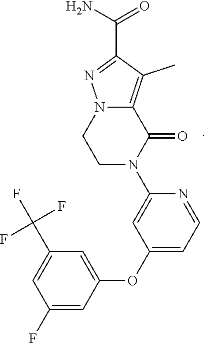 [SMILES: Cc1c(C(N)=O)nn2c1C(=O)N(c1cc(Oc3cc(F)cc(C(F)(F)F)c3)ccn1)CC2]

111. The compound of any one of claims 1 to 3, or a pharmaceutically acceptable salt thereof, wherein the compound has the structure:

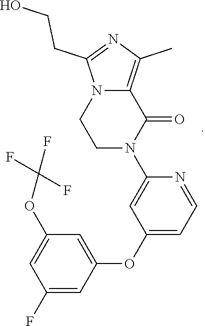 [SMILES: Cc1nc(CCO)n2c1C(=O)N(c1cc(Oc3cc(F)cc(OC(F)(F)F)c3)ccn1)CC2]

112. The compound of any one of claims 1 to 3, or a pharmaceutically acceptable salt thereof, wherein the compound has the structure:

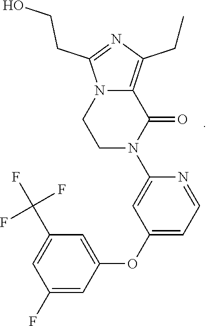 [SMILES: CCc1nc(CCO)n2c1C(=O)N(c1cc(Oc3cc(F)cc(C(F)(F)F)c3)ccn1)CC2]

113. The compound of any one of claims 1 to 3, or a pharmaceutically acceptable salt thereof, wherein the compound has the structure:

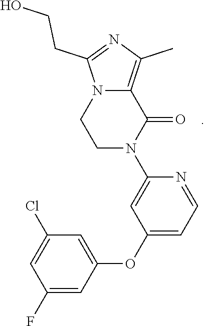 [SMILES: Cc1nc(CCO)n2c1C(=O)N(c1cc(Oc3cc(F)cc(Cl)c3)ccn1)CC2]

114. The compound of any one of claims 1 to 3, or a pharmaceutically acceptable salt thereof, wherein the compound has the structure:

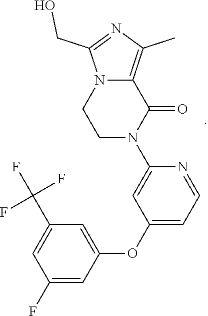 [SMILES: Cc1nc(CO)n2c1C(=O)N(c1cc(Oc3cc(F)cc(C(F)(F)F)c3)ccn1)CC2]

115. The compound of any one of claims 1 to 3, or a pharmaceutically acceptable salt thereof, wherein the compound has the structure:

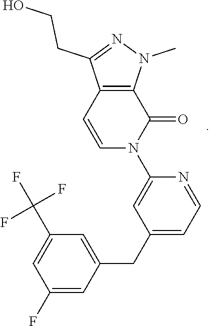 [SMILES: Cn1nc(CCO)c2ccn(-c3cc(Cc4cc(F)cc(C(F)(F)F)c4)ccn3)c(=O)c21]

116. The compound of any one of claims 1 to 3, or a pharmaceutically acceptable salt thereof, wherein the compound has the structure:

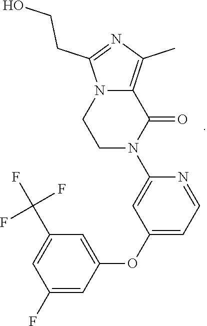 [SMILES: Cc1nc(CCO)n2c1C(=O)N(c1cc(Oc3cc(F)cc(C(F)(F)F)c3)ccn1)CC2]

117. A pharmaceutical composition comprising a compound of any one of claims 1-116, or a pharmaceutically acceptable salt thereof, and a pharmaceutically acceptable carrier.

118. A method of modulating GPR52 activity, the method comprising contacting a cell expressing GPR52 with a compound of any one of claims 1-116, or a pharmaceutically acceptable salt thereof.

119. The method of claim 118, wherein the contacting is in vitro.

120. The method of claim 118, wherein the contacting is in vivo.

121. A method for treating a disorder selected from the group consisting of psychiatric disorders; neuropsychiatric disorders; neurodegenerative disorders; psychotic disorders; cognitive disorders; neurocognitive disorders; extrapyramidal disorders; movement disorders; motor disorders; hyperkinetic movement disorders; catatonia; mood disorders; depressive disorders; anxiety disorders; obsessive-compulsive disorder (OCD); autism spectrum disorders; hypothalamic disorders; pituitary disorders; prolactin-related disorders; trauma- or stressor-related disorders; disruptive, impulse-control or conduct disorders; sleep-wake disorders; substance-related disorders; addictive disorders; behavioural disorders; hypofrontality; abnormalities in the tuberoinfundibular, mesolimbic, mesocortical, or nigrostriatal pathways; decreased activity in the striatum; cortical dysfunction; neurocognitive dysfunction, and related conditions or symptoms, the method comprising administering to a patient in need of such treatment a compound of any one of claims 1-116, or a pharmaceutically acceptable salt thereof or a pharmaceutical composition as claimed in claim 117.

122. A method for treating a disorder selected from the group consisting of schizophrenia, depression, attention-deficit hyperactivity disorder (ADHD), generalised anxiety disorder, OCD, panic disorder, bipolar disorder, addiction/impulse-control disorders, autism spectrum disorders, psychosis, anhedonia, agitation, Alzheimer's disease, Parkinson's disease, Huntington's disease, vascular dementia, Lewy body disease, frontotemporal dementia, Tourette's syndrome, hyperprolactinemia, pituitary adenoma, prolactinoma, craniopharyngioma, Cushing's disease, diabetes insipidus, non-functioning tumors, obesity, posttraumatic stress disorder (PTSD), akathisia, athetosis, ataxia, ballismus, hemiballismus, chorea, choreoathetosis, dyskinesia, tardive dyskinesia, neuroleptic-induced dyskinesia, myoclonus, mirror movement disorder, paroxysmal kinesigenic dyskinesia, restless legs syndrome, spasms, stereotypic movement disorder, tic disorder, tremor, Wilson's disease, schizotypal personality disorder, delusional disorder, brief psychotic disorder, schizophreniform disorder, schizoaffective disorder, substance- or medication-induced psychotic disorder, delusions, hallucinations, disorganized thinking, grossly disorganized or abnormal motor behavior, major depressive disorder, bipolar I disorder, bipolar II disorder, cyclothymic disorder, substance- or medication-induced bipolar and related disorders, bipolar and related disorders due to another medical condition, separation anxiety disorder, selective mutism, specific phobia, social anxiety disorder, agoraphobia, anxiety disorders due to another medical condition, major neurocognitive disorder, minor neurocognitive disorder, amnesia, dementia, developmental coordination disorder, post-stroke effects, dentatorubral-pallidoluysian atrophy, diminished emotional expression, avolition, alogia, and asociality, the method comprising administering to a patient in need of such treatment a compound of any one of claims 1-116, or a pharmaceutically acceptable salt thereof or a pharmaceutical composition as claimed in claim 117.
